(19) United States
(12) Patent Application Publication
(10) Pub. No.: US 2009/0305284 A1
(43) Pub. Date: Dec. 10, 2009

(54) Methods for Identifying Risk of Breast Cancer and Treatments Thereof

(75) Inventors: Richard R. Roth, San Diego, CA (US); Andreas Braun, San Diego, CA (US); Stefan M. Kammerer, San Diego, CA (US); Matthew Roberts Nelson, Chapel Hill, NC (US); Rikard Henry Reneland, Knivsta (SE); Carolyn R. Hoyal-Wrightson, San Diego, CA (US)

Correspondence Address: GRANT ANDERSON LLP;C/O PORTFOLIOIP, PO BOX 52050, MINNEAPOLIS, MN, 55402, US

(73) Assignee: SEQUENOM, INC., San Diego, CA (US)

(21) Appl. No.: 12/479,478

(22) Filed: Jun. 5, 2009

Related U.S. Application Data
(63) Continuation of application No. 10/857,780, filed on May 27, 2004, now abandoned.
Application No. 10/857,780 is a continuation-in-part of application No. 10/723,681, filed on Nov. 25, 2003, now Pat. No. 7,510,835.
(60) Provisional application No. 60/490,234, filed on Jul. 24, 2003.
(60) Provisional application No. 60/525,239, filed on Nov. 25, 2003.

(51) Int. Cl. C12Q 1/68

(52) U.S. Cl. 435/6

(57) ABSTRACT

Provided herein are methods for identifying risk of breast cancer in a subject and/or a subject at risk of breast cancer, reagents and kits for carrying out the methods, methods for identifying candidate therapeutics for treating breast cancer, and therapeutic methods for treating breast cancer in a subject. These embodiments are based upon an analysis of polymorphic variations in nucleotide sequences within the human genome.

### RELATED PATENT APPLICATIONS

This patent application claims the benefit of provisional patent application No. 60/490,234 filed Jul. 24, 2003, having attorney docket number 524593004101. This patent application is a continuation of U.S. patent application Ser. No. 10/857,780, filed on May 27, 2004, entitled “Methods for Identifying Risk of Breast Cancer and Treatments Thereof,” which is a continuation in part of U.S. patent application Ser. No. 10/723,681 filed on Nov. 25, 2003, entitled “Methods for identifying risk of breast cancer and treatments thereof,” naming Richard B. Roth et al. as inventors, and having attorney docket no. 524592006900. U.S. patent application Ser. Nos. 10/723,670, 10/723,518, 10/722,939 and 10/723,683, having attorney docket numbers 524592006700, 524592006800, 524592007100 and 524592007200, respectively, filed on Nov. 25, 2003, name Richard B. Roth et al. as inventors, and have the same title as U.S. patent application Ser. No. 10/723,681, are related. This patent application also claims the benefit of U.S. patent application No. 60/525,239 filed on Nov. 25, 2003, naming Matthew R. Nelson as an inventor, entitled “Disease risk prediction with associated single nucleotide polymorphisms,” and having attorney docket number 524593006400. Each of these patent applications is hereby incorporated herein by reference in its entirety, including all drawings, cited publications and documents.

### FIELD OF THE INVENTION

The invention relates to genetic methods for identifying risk of breast cancer and treatments that specifically target the disease.

### SEQUENCE LISTING DISCLOSURE

In accordance with 37 C.F.R. § 1.52(e)(5), the material in the text file containing the sequence listing is hereby incorporated by reference in its entirety. The text file containing the sequence listing is named “SEQ-4069-CP.txt,” is 2.23 megabytes in size and was created on Dec. 8, 2004.

### BACKGROUND

Breast cancer is the third most common cancer, and the most common cancer in women, as well as a cause of disability, psychological trauma, and economic loss. Breast cancer is the second most common cause of cancer death in women in the United States, in particular for women between the ages of 15 and 54, and the leading cause of cancer-related death (Forbes, Seminars in Oncology, vol. 24(1), Suppl 1, 1997: pp. S1-20-S1-35). Indirect effects of the disease also contribute to the mortality from breast cancer including consequences of advanced disease, such as metastases to the bone or brain. Complications arising from bone marrow suppression, radiation fibrosis and neutropenic sepsis, collateral effects from therapeutic interventions, such as surgery, radiation, chemotherapy, or bone marrow transplantation-also contribute to the morbidity and mortality from this disease.

While the pathogenesis of breast cancer is unclear, transformation of normal breast epithelium to a malignant phenotype may be the result of genetic factors, especially in women under thirty (Miki, et al., Science, 266: 66-71 (1994)). However, it is likely that other, non-genetic factors also have a significant effect on the etiology of the disease. Regardless of its origin, breast cancer morbidity increases significantly if it is not detected early in its progression. Thus, considerable efforts have focused on the elucidation of early cellular events surrounding transformation in breast tissue. Such efforts have led to the identification of several potential breast cancer markers. For example, alleles of the BRCA1 and BRCA2 genes have been linked to hereditary and early-onset breast cancer (Wooster, et al., Science, 265: 2088-2090 (1994)). However, BRCA1 is limited as a cancer marker because BRCA1 mutations fail to account for the majority of breast cancers (Ford, et al., British J. Cancer, 72: 805-812 (1995)). Similarly, the BRCA2 gene, which has been linked to forms of hereditary breast cancer, accounts for only a small portion of total breast cancer cases.

### SUMMARY

It has been discovered that certain polymorphic variations in human genomic DNA are associated with the occurrence of breast cancer. In particular, polymorphic variants in loci containing ICAM, MAPK10, KIAA0861, NUMA1/FLJ20625/LOC220074 (hereafter referred to as “NUMA1”), HT014/LOC148902/LYPLA2/GALE (hereafter referred to as “GALE”), DPF3 and LOC145197 regions in human genomic DNA have been associated with risk of breast cancer.

Thus, featured herein are methods for identifying a subject at risk of breast cancer and/or a risk of breast cancer in a subject, which comprises detecting the presence or absence of one or more polymorphic variations associated with breast cancer in genomic regions described herein in a human nucleic acid sample. In an embodiment, two or more polymorphic variations are detected in two or more regions selected from the group consisting of ICAM, MAPK10, KIAA0861, NUMA1, GALE, DPF3 and LOC145197. In certain embodiments, 3 or more, or 4, 5, 6, 7, 8, 9, 10, 11, 12, 13, 14, 15, 16, 17, 18, 19 or 20 or more polymorphic variants are detected. In specific embodiments, the group of polymorphic variants detected comprise or consist of polymorphic variants in ICAM, MAPK10, KIAA0861, NUMA1, GALE, DPF3 and/or LOC145197, such as position 44247 in SEQ ID NO: 1 (ICAM), position 36424 in SEQ ID NO: 2 (MAPK10), position 48563 in SEQ ID NO: 3 (KIAA0861), position 49002 in SEQ ID NO: 4 (NUMA1) and position 174 in SEQ ID NO: 5 (GALE), for example.

Also featured are nucleic acids that include one or more polymorphic variations associated with the occurrence of breast cancer, as well as polypeptides encoded by these nucleic acids. Further, provided is a method for identifying a subject at risk of breast cancer and then prescribing to the subject a breast cancer detection procedure, prevention procedure and/or a treatment procedure. In addition, provided are methods for identifying candidate therapeutic molecules for treating breast cancer and related disorders, as well as methods for treating breast cancer in a subject by diagnosing breast cancer in the subject and treating the subject with a suitable treatment, such as administering a therapeutic molecule.

Also provided are compositions comprising a breast cancer cell and/or ICAM, MAPK10, KIAA0861, NUMA1, GALE, DPF3 or LOC145197 nucleic acid with a RNAi, siRNA, antisense DNA or RNA, or ribozyme nucleic acid designed from a ICAM, MAPK10, KIAA0861, NUMA1, GALE, DPF3 or LOC145197 nucleotide sequence. In an embodiment, the nucleic acid is designed from a ICAM, MAPK10, KIAA0861, NUMA1, GALE, DPF3 or LOC145197 nucleotide sequence that includes one or more breast cancer associated polymorphic variations, and in some instances, specifically interacts with such a nucleotide sequence. Further, provided are arrays of nucleic acids bound to a solid surface, in which one or more nucleic acid molecules of the array have a ICAM, MAPK10, KIAA0861, NUMA1, GALE, DPF3 or LOC145197 nucleotide sequence, or a fragment or substantially identical nucleic acid thereof, or a complementary nucleic acid of the foregoing. Featured also are compositions comprising a breast cancer cell and/or a ICAM, MAPK10, KIAA0861, NUMA1, GALE, DPF3 or LOC145197 polypeptide, with an antibody that specifically binds to the polypeptide. In an embodiment, the antibody specifically binds to an epitope in the polypeptide that includes a non-synonymous amino acid modification associated with breast cancer (e.g., results in an amino acid substitution in the encoded polypeptide associated with breast cancer). In certain embodiments, the antibody specifically binds to an epitope that comprises a proline at amino acid position 352, an alanine at amino acid position 348, or a glycine at amino acid position 241 in an ICAM5 polypeptide.

### BRIEF DESCRIPTION OF THE FIGURES

FIGS. 1A-1G show proximal SNPs in ICAM, MAPK10, KIAA0861, NUMA1, GALE, DPF3 and LOC145197 regions of genomic DNA, respectively. The position of each SNP on the chromosome is shown on the x-axis and the y-axis provides the negative logarithm of the p-value comparing the estimated allele to that of the control group. Also shown in the figure are exons and introns of the genes in the approximate chromosomal positions.

FIGS. 2A-2G show results of an odds-ratio meta analysis for ICAM, MAPK10, KIAA0861 NUMA1, GALE, DPF3 and LOC114197 regions.

FIG. 3 shows effects of ICAM-directed siRNA on cancer cell proliferation.

### DETAILED DESCRIPTION

It has been discovered that polymorphic variations in the ICAM, MAPK10, KIAA0861, NUMA1, GALE, DPF3 and LOC145197 regions described herein are associated with an increased risk of breast cancer.

All ICAM proteins are type I transmembrane glycoproteins, contain 2-9 immunoglobulin-like C2-type domains, and bind to the leukocyte adhesion LFA-1 protein. The proteins are members of the intercellular adhesion molecule (ICAM) family. The gene ICAM1 (intercellular adhesion molecule-1) is also known as human rhinovirus receptor, BB2, CD54. and cell surface glycoprotein P3.58. ICAM1 has been mapped to chromosomal position 19p13.3-p13.2. ICAM1 (CD54) typically is expressed on endothelial cells and cells of the immune system. ICAM1 binds to integrins of type CD11a/CD18, or CD11b/CD18. ICAM1 is also exploited by Rhinovirus as a receptor.

The gene ICAM4 (intercellular adhesion molecule 4) is also known as the Landsteiner-Wiener blood group or LW. ICAM4 has been mapped to 19p13.2-cen. The protein encoded by this gene is a member of the intercellular adhesion molecule (ICAM) family. A glutamine to arginine polymorphism in this protein is responsible for the Landsteiner-Wiener blood group system (GLN=WB(A); ARG=WB(B). This gene consists of 3 exons and alternative splicing generates 2 transcript variants.

The gene ICAM5 (intercellular adhesion molecule 5) is also known as telencephalin. ICAM5 has been mapped to 19p13.2. The protein encoded by the gene is expressed on the surface of telencephalic neurons and displays two types of adhesion activity, homophilic binding between neurons and heterophilic binding between neurons and leukocytes. It may be a critical component in neuron-microglial cell interactions in the course of normal development or as part of neurodegenerative diseases.

The gene MAPK10 also is known as JNK3, JNK3A, PRKM10, p493F12, FLJ12099, p54bSAPK MAP kinase, c-Jun kinase 3, JNK3 alpha protein kinase, c-Jun N-terminal kinase 3, stress activated protein kinase JNK3, stress activated protein kinase beta. MAPK10 has been mapped to chromosomal position 4q22.1-q23. The protein encoded by this gene is a member of the MAP kinase family. MAP kinases act as an integration point for multiple biochemical signals, and are involved in a wide variety of cellular processes such as proliferation, differentiation, transcription regulation and development. This protein is a neuronal-specific form of c-Jun N-terminal kinases (JNKs). Through its phosphorylation and nuclear localization, this kinase plays regulatory roles in the signaling pathways during neuronal apoptosis. Beta-arrestin 2, a receptor-regulated MAP kinase scaffold protein, is found to interact with, and stimulate the phosphorylation of this kinase by MAP kinase kinase 4 (MKK4). Cyclin-dependent kinase 5 can phosphorylate, and inhibit the activity of this kinase, which may be important in preventing neuronal apoptosis. Four alternatively spliced transcript variants encoding distinct isoforms have been reported.

The gene KIAA0861 is a Rho family guanine-nucleotide exchange factor. KIAA0861 has been mapped to chromosomal position 3q27.3. KIAA0861 is a Rho family nucleotide exchange factor homolog that modulates the activity of Rho family GTPases, which control numerous cell functions, including cell growth, adhesion, movement and shape. RhoC GTPase is overexpressed in invasive (inflammatory) breast cancers.

The gene FLJ20625 has been mapped to chromosomal position 11q13.3. The gene encoding LOC220074 also is known as Hypothetical 55.1 kDa protein F09G8.5 in chromosome III and has been mapped to chromosomal position 11q13.3.

The gene HT014 has been mapped to chromosomal position 1p36.11. The gene LYPLA2 (lysophospholipase II) also is known as APT-2, DJ886K2.4 and acyl-protein thioesterase and has been mapped to chromosomal position 1p36.12-p35.1. Lysophospholipases are enzymes that act on biological membranes to regulate the multifunctional lysophospholipids. There are alternatively spliced transcript variants described for this gene but the full length nature is not known yet.

The gene GALE (galactose-4-epimerase, UDP-) also is known as galactowaldenase UDP galactose-4-epimerase and has been mapped to chromosomal position 1p36-p35. This gene encodes UDP-galactose-4-epimerase which catalyzes 2 distinct but analogous reactions: the epimerization of UDP-glucose to UDP-galactose, and the epimerization of UDP-N-acetylglucosamine to UDP-N-acetylgalactosamine. The bifunctional nature of the enzyme has the important metabolic consequence that mutant cells (or individuals) are dependent not only on exogenous galactose, but also on exogenous N-acetylgalactosamine for necessary precursor for the synthesis of glycoproteins and glycolipids. The missense mutations in the GALE gene result in the epimerase-deficiency galactosemia.

The gene DPF3 (D4, zinc and double PHD fingers, family 3) also is known as CERD4, cer-d4, FLJ14079, and 2810403B03Rik. DPF3 is a Rho family guanine-nucleotide exchange factor. DPF3 has been mapped to chromosomal position 14q24.3-q31.1.

Breast Cancer and Sample Selection

Breast cancer is typically described as the uncontrolled growth of malignant breast tissue. Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory breast cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body.

As used herein, the term “breast cancer” refers to a condition characterized by anomalous rapid proliferation of abnormal cells in one or both breasts of a subject. The abnormal cells often are referred to as “neoplastic cells,” which are transformed cells that can form a solid tumor. The term “tumor” refers to an abnormal mass or population of cells (i.e. two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize. In breast cancer, neoplastic cells may be identified in one or both breasts only and not in another tissue or organ, in one or both breasts and one or more adjacent tissues or organs (e.g. lymph node), or in a breast and one or more non-adjacent tissues or organs to which the breast cancer cells have metastasized.

The term “invasion” as used herein refers to the spread of cancerous cells to adjacent surrounding tissues. The term “invasion” often is used synonymously with the term “metastasis,” which as used herein refers to a process in which cancer cells travel from one organ or tissue to another non-adjacent organ or tissue. Cancer cells in the breast(s) can spread to tissues and organs of a subject, and conversely, cancer cells from other organs or tissue can invade or metastasize to a breast. Cancerous cells from the breast(s) may invade or metastasize to any other organ or tissue of the body. Breast cancer cells often invade lymph node cells and/or metastasize to the liver, brain and/or bone and spread cancer in these tissues and organs. Breast cancers can spread to other organs and tissues and cause lung cancer, prostate cancer, colon cancer, ovarian cancer, cervical cancer, gastrointestinal cancer, pancreatic cancer, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, ovarian cancer, neuroblastoma, myeloma, various types of head and neck cancer, acute lymphoblastic leukemia, acute myeloid leukemia, Ewing sarcoma and peripheral neuroepithelioma, and other carcinomas, lymphomas, blastomas, sarcomas, and leukemias.

Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory Breast Cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body. As used herein, the term “breast cancer” may include both Inflammatory Breast Cancer and Recurrent Breast Cancer.

In an effort to detect breast cancer as early as possible, regular physical exams and screening mammograms often are prescribed and conducted. A diagnostic mammogram often is performed to evaluate a breast complaint or abnormality detected by physical exam or routine screening mammography. If an abnormality seen with diagnostic mammography is suspicious, additional breast imaging (with exams such as ultrasound) or a biopsy may be ordered. A biopsy followed by pathological (microscopic) analysis is a definitive way to determine whether a subject has breast cancer. Excised breast cancer samples often are subjected to the following analyses: diagnosis of the breast tumor and confirmation of its malignancy; maximum tumor thickness; assessment of completeness of excision of invasive and in situ components and microscopic measurements of the shortest extent of clearance; level of invasion; presence and extent of regression; presence and extent of ulceration; histological type and special variants; pre-existing lesion; mitotic rate; vascular invasion; neurotropism; cell type; tumor lymphocyte infiltration; and growth phase.

The stage of a breast cancer can be classified as a range of stages from Stage 0 to Stage IV based on its size and the extent to which it has spread. The following table summarizes the stages:

TABLE A

| | | | Metastasis |
|---|---|---|---|
| Stage | Tumor Size | Lymph Node Involvement | (Spread) |
| I | Less than 2 cm | No | No |
| II | Between 2-5 cm | No or in same side of breast | No |
| III | More than 5 cm | Yes, on same side of breast | No |
| IV | Not applicable | Not applicable | Yes |

Stage 0 cancer is a contained cancer that has not spread beyond the breast ductal system. Fifteen to twenty percent of breast cancers detected by clinical examinations or testing are in Stage 0 (the earliest form of breast cancer). Two types of Stage 0 cancer are lobular carcinoma in situ (LCIS) and ductal carcinoma in situ (DCIS). LCIS indicates high risk for breast cancer. Many physicians do not classify LCIS as a malignancy and often encounter LCIS by chance on breast biopsy while investigating another area of concern. While the microscopic features of LCIS are abnormal and are similar to malignancy, LCIS does not behave as a cancer (and therefore is not treated as a cancer). LCIS is merely a marker for a significantly increased risk of cancer anywhere in the breast. However, bilateral simple mastectomy may be occasionally performed if LCIS patients have a strong family history of breast cancer. In DCIS the cancer cells are confined to milk ducts in the breast and have not spread into the fatty breast tissue or to any other part of the body (such as the lymph nodes). DCIS may be detected on mammogram as tiny specks of calcium (known as microcalcifications) 80% of the time. Less commonly DCIS can present itself as a mass with calcifications (15% of the time); and even less likely as a mass without calcifications (<5% of the time). A breast biopsy is used to confirm DCIS. A standard DCIS treatment is breast-conserving therapy (BCT), which is lumpectomy followed by radiation treatment or mastectomy. To date, DCIS patients have chosen equally among lumpectomy and mastectomy as their treatment option, though specific cases may sometimes favor lumpectomy over mastectomy or vice versa.

In Stage I, the primary (original) cancer is 2 cm or less in diameter and has not spread to the lymph nodes. In Stage IIA, the primary tumor is between 2 and 5 cm in diameter and has not spread to the lymph nodes. In Stage IIB, the primary tumor is between 2 and 5 cm in diameter and has spread to the axillary (underarm) lymph nodes; or the primary tumor is over 5 cm and has not spread to the lymph nodes. In Stage IIIA, the primary breast cancer of any kind that has spread to the axillary (underarm) lymph nodes and to axillary tissues. In Stage IIIB, the primary breast cancer is any size, has attached itself to the chest wall, and has spread to the pectoral (chest) lymph nodes. In Stage IV, the primary cancer has spread out of the breast to other parts of the body (such as bone, lung, liver, brain). The treatment of Stage IV breast cancer focuses on extending survival time and relieving symptoms.

Based in part upon selection criteria set forth above, individuals having breast cancer can be selected for genetic studies. Also, individuals having no history of cancer or breast cancer often are selected for genetic studies. Other selection criteria can include: a tissue or fluid sample is derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy).

Provided herein is a set of blood samples and a set of corresponding nucleic acid samples isolated from the blood samples, where the blood samples are donated from individuals diagnosed with breast cancer. The sample set often includes blood samples or nucleic acid samples from 100 or more, 150 or more, or 200 or more individuals having breast cancer, and sometimes from 250 or more, 300 or more, 400 or more, or 500 or more individuals. The individuals can have parents from any place of origin, and in an embodiment, the set of samples are extracted from individuals of German paternal and German maternal ancestry. The samples in each set may be selected based upon five or more criteria and/or phenotypes set forth above.

Polymorphic Variants Associated with Breast Cancer

A genetic analysis provided herein linked breast cancer with polymorphic variants in the ICAM, MAPK10, KIAA0861, NUMA1, GALE, DPF3 and LOC145197 regions of the human genome disclosed herein. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

Furthermore, a genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of breast cancer.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. For example, a thymine at a particular position in SEQ ID NO: 1 can be reported as an adenine from the complementary strand. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

In the genetic analysis that associated breast cancer with the polymorphic variants described hereafter, samples from individuals having breast cancer and individuals not having cancer were allelotyped and genotyped. The term “genotyped” as used herein refers to a process for determining a genotype of one or more individuals, where a “genotype” is a representation of one or more polymorphic variants in a population. Genotypes may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

It was determined that polymorphic variations associated with an increased risk of breast cancer existed in ICAM, MAPK10, KIAA0861, NUMA1, GALE, DPF3 and LOC145197 nucleotide sequences. Polymorphic variants in and around the ICAM, MAPK10, KIAA0861, NUMA1, GALE, DPF3 and LOC145197 loci were tested for association with breast cancer. In the ICAM locus, these included polymorphic variants at positions selected from the group consisting of rs2884487, rs2358580, rs2304236, rs1059840, rs1059843, rs11115, rs1059849, rs1059855, rs5030386, rs5030339, rs5030387, rs5030388, rs1799766, rs5030389, rs5490, rs11575070, rs5030340, rs5030390, rs5030391, rs3093035, rs11667983, rs5030341, rs5030342, rs5030343, rs5030344, rs5030347, rs5030348, rs5030349, rs5030350, rs5030351, rs5491, rs5030352, rs5030353, rs10420063, rs11879117, rs5030354, rs5030355, rs281428, rs5030358, rs5030359, rs5030392, rs5030393, rs5030360, rs5030394, rs281429, rs5030361, rs5030362, rs281430, rs281431, rs5030395, rs5827095, rs281432, rs5030364, rs5030365, rs5030368, rs5030369, rs3073809, rs2358581, rs7258215, rs5030371, rs5030372, rs281433, rs5030374, rs5030375, rs5030397, rs281434, rs12462944, rs5030398, rs5030378, rs12459133, rs5030399, rs5492, rs1800019, rs1799969, rs5493, rs5030381, rs5494, rs3093033, rs5495, rs1801714, rs2071441, rs5496, rs5497, rs5030382, rs5030400, rs2071440, rs5499, rs3093032, rs1057981, rs5500, rs5501, rs5030383, rs281436, rs923366, rs281437, rs3093030, rs5030384, rs5030385, rs3810159, rs281438, rs3093029, rs2735442, rs2569693, rs281439, rs281440, rs2569694, rs11575073, rs2569695, rs2075741, rs11575074, rs2569696, rs2735439, rs2569697, rs2075742, rs2569698, rs11669397, rs901886, rs885742, rs2569699, rs11549918, rs2569700, rs2228615, rs2569701, rs2569702, rs2735440, rs2569703, rs10418913, rs1056536, rs2569704, rs11673661, rs10402760, rs2569706, rs2569707, rs2436545, rs2436546, rs2916060, rs2916059, rs2916058, rs2569708, rs735747, rs885743, rs710845, rs2569709, rs2569710, rs2569711, rs2569712, rs12610026, rs4804129, rs12150978, rs439843, rs892188, rs2291473, rs281416, rs281417, rs882589, rs1048941, rs281418, rs430092, rs368835, rs2358583, rs378395, rs395782, rs1045384, rs281427, rs3745264, rs281426, rs281425, rs281424, rs281423, rs281422, rs281420, rs3745263, rs3745262, rs3745261, rs3181049, rs281412, rs3181048, rs2230399, rs2278442, rs3181047, rs3181046, rs2304237, rs281413, rs1058154, rs3176769, rs2304238, rs2304239, rs2304240, rs3176768, rs3176767, rs3176766, rs281414, rs281415, position 45003 of SEQ ID NO: 1, and position 47504 of SEQ ID NO: 1. Polymorphic variants at the following positions in particular were associated with an increased risk of breast cancer: rs11115, rs1059849, rs5030391, rs5030382, rs3093030, rs2569693, rs2075741, rs901886, rs11549918, rs2228615, rs2569702, rs2569703, rs10402760, rs710845, rs892188, and rs281426. At these positions in SEQ ID NO: 1, a cytosine at position 11942, an adenine at position 12054, a guanine at position 24037, an adenine at position 37083, a cytosine at position 38803, a cytosine at position 41304, a guanine at position 42498, a thymine at position 43531, a cytosine at position 44338, a guanine at position 44768, a thymine at position 45347, a cytosine at position 45627, a thymine at position 47265, a cytosine at position 48569, a cytosine at position 51193, a guanine at position 72451, in particular were associated with risk of breast cancer. Also, a proline at amino acid position 352 or an alanine at amino acid position 348 in SEQ ID NO: 15 were in particular associated with an increased risk of breast cancer.

In the MAPK10 locus, these included polymorphic variants at positions selected from the group consisting of rs2575681, rs2575680, rs2589505, rs2589504, rs2164538, rs2575679, rs10305, rs2869408, rs2904086, rs934648, rs2589511, rs2060589, rs2164537, rs2575678, rs2575677, rs2589510, rs2589509, rs2164536, rs2164535, rs1946734, rs2589525, rs2589523, rs3755970, rs2575675, rs1202, rs1201, rs2589516, rs2575674, rs2589515, rs3733367, rs958, rs2589506, rs1436524, rs2575672, rs2589518, rs3775164, rs2589514, rs3775166, rs3775167, rs3822035, rs3775168, rs3775169, rs2043650, rs2043649, rs3775170, rs1541998, rs2282599, rs2282598, rs2282597, rs3775171, rs3775172, rs3775173, rs3775174, rs1469870, rs1436522, rs1946733, rs983362, rs3755971, rs3822036, rs3775175, rs1436525, rs3822037, rs3775176, rs993593, rs1436527, rs1436529, rs3775180, rs3775181, rs3775182, rs3775183, rs3775184, rs733245, rs3775185, rs1561154, rs3775186, rs3775187, rs1010778, rs2282596, rs2282595, rs2118044, rs1469869, position 21751 of SEQ ID NO: 2, position 30965 of SEQ ID NO: 2, position 36838 of SEQ ID NO: 2, position 36889 of SEQ ID NO: 2, position 38639 of SEQ ID NO: 2, position 38865 of SEQ ID NO: 2, position 38885 of SEQ ID NO: 2, position 38943 of SEQ ID NO: 2, position 39035 of SEQ ID NO: 2, position 39046 of SEQ ID NO: 2, and position 110704 of SEQ ID NO: 2. Polymorphic variants at the following positions in particular were associated with an increased risk of breast cancer: rs2575677, rs2575674, rs1436524, rs2575672, rs2589518, rs2589514, rs3775166, MAPK10-AB, rs3775168, rs2043650, rs2043649, rs1541998, position 38865 of SEQ ID NO: 2, position 39035 of SEQ ID NO: 2, position 39046 of SEQ ID NO: 2, rs1469870, and rs3775176. At these positions in SEQ ID NO: 2, a guanine at position 13918, an adenine at position 23841, an adenine at position 26072, a thymine at position 26376, an adenine at position 26614, an adenine at position 26827, a cytosine at position 27084, an adenine at position 30965, an adenine at position 32979, an adenine at position 35142, a thymine at position 35237, a cytosine at position 36439, a thymine at position 38865, a thymine at position 39035, a cytosine at position 39046, a cytosine at position 46191, and an adenine at position 62587, in particular were associated with risk of breast cancer.

In the KIAA0861 locus, these included polymorphic variants at positions selected from the group consisting of rs3811728, rs3811729, rs602646, rs488277, rs1629673, rs670232, rs575326, rs575386, rs684846, rs471365, rs 496251, rs831246, rs831247, rs512071, rs1502761, rs681516, rs683302, rs619424, rs620722, rs529055, rs664010, rs678454, rs2653845, rs472795, rs507079, rs534333, rs535298, rs536213, rs831245, rs639690, rs684174, rs571761, rs1983421, rs4630966, rs2314415, rs6788196, rs2103062, rs9827084, rs9864865, rs6804951, rs6770548, rs1403452, rs7609994, rs9838250, rs9863404, rs903950, rs6787284, rs2017340, rs2001449, rs1317288, rs7635891, rs10704581, rs11371910, rs10937118, rs7642053, rs3821522, rs2029926, rs1390831, rs7643890, rs11925606, rs9826325, rs6800429, rs6803368, rs1353566, rs2272115, rs2272116, rs3732603, rs940055, rs2314730, rs2030578, rs2049280, rs3732602, rs2293203, rs7639705, and position 13507 of SEQ ID NO: 3. Polymorphic variants at the following positions in particular were associated with an increased risk of breast cancer: rs4630966, rs9827084, rs9864865, rs6804951, rs6770548, rs1403452, and rs2001449. At these positions in SEQ ID NO: 3, a cytosine at position 41716, a guanine at position 44775, a guanine at position 44962, a cytosine at position 45317, a guanine at position 45712, a thymine at position 45941, and a cytosine at position 48849, in particular were associated with risk of breast cancer. Also, an alanine at amino acid position 819 in SEQ ID NO: 22 (or position 902 in SEQ ID NO: 23) was in particular associated with an increased risk of breast cancer.

In the NUMA1 locus, these included polymorphic variants at positions corresponding to these selected from the group consisting of rs4945392, rs7938496, rs7926550, rs7945374, rs7949480, rs7102523, rs7121260, rs7131230, rs7128317, rs2276385, rs2276384, rs2276383, rs1892921, rs1892920, rs7942626, rs7124429, rs7114081, rs7125718, rs1055452, rs3829215, rs1541306, rs3814722, rs1939240, rs5743655, rs5743656, rs5743657, rs3814721, rs5743658, rs5743659, rs5743660, rs5743661, rs5743662, rs2298455, rs5743664, rs5743665, rs5743667, rs5743668, rs5743669, rs1573503, rs5743670, rs5743671, rs1892919, rs5743672, rs2735786, rs5743673, rs5743674, rs1062452, rs5743675, rs5743676, rs760246, rs11345794, rs5743677, rs5743678, rs14537, rs3168177, rs5743679, rs1541304, rs1053725, rs5743680, rs949324, rs949323, rs5743681, rs5743682, rs5743683, rs5743684, rs5743685, rs2845857, rs2852365, rs11538641, rs11538639, rs11235417, rs11538644, rs11538643, rs2845858, rs2155146, rs2852364, rs2845859, rs2845860, rs11538642, rs7949430, rs7938674, rs2735787, rs11538640, rs10736784, rs10736785, rs11826059, rs2503, rs2155145, rs4477459, position 26334 of SEQ ID NO: 4, rs5019605, rs5019604, rs5019603, rs3793941, rs11235418, rs3793940, rs2032353, rs3934448, rs7951267, rs1053603, rs1053602, rs11235419, rs1053601, rs3750913, rs3750912, rs1053600, rs1057992, rs2298789, rs10898813, position 34941 of SEQ ID NO: 4, rs949325, position 35629 of SEQ ID NO: 4, rs949326, rs2298456, rs7949845, rs7949989, rs1573500, rs4945411, rs11235422, rs10128658, rs2298457, rs3838779, rs1573501, rs11235424, rs10898814, rs7930142, rs7930544, rs7930721, rs7930722, rs6592456, rs11235425, rs6592457, rs10898815, rs11235426, rs10898816, rs1939247, rs1939246, rs1939245, rs1939244, rs1939243, rs4245463, rs4944258, rs7926751, rs12282917, rs12282918, rs7937582, rs10898817, rs7480015, rs7123992, rs1573502, rs7127865, rs4945426, rs11235428, rs7122489, rs1894003, rs1548348, rs1892923, rs5792570, rs7115200, rs4945430, rs11235429, rs1939242, rs9666346, rs7101553, rs7124000, rs12291664, rs12291778, rs12291781, rs12291787, rs12291788, rs12291833, rs10793016, rs5792571, rs12293529, rs4338555, rs11235431, rs6592458, rs4945434, rs4945435, rs11307657, rs5792573, rs1894004, rs12276164, rs4378421, rs7116495, rs7101701, rs10736786, rs11608165, rs645603, rs661290, rs7122209, rs12417471, rs541228, rs12273666, rs2511074, rs3018311, rs3018289, rs679926, rs567026, rs564294, rs678193, rs677279, rs560777, rs676721, rs7106529,rs11602304, rs585228,rs578957, rs7110215, rs3133233, rs3133230, rs12576024, rs674319, rs675185, rs5792574, rs12275272, rs10400327, rs612255, rs7101643, rs575871, rs547208, rs2511075, rs642573, rs482197, rs656640, rs671681, rs541022, rs951586, rs2511076, rs11235432, rs3018308, rs11606798, rs1791544, rs3018304, rs10751193, rs4945470, rs3018291, rs2511120, rs671132, rs4945475, rs3018292, rs642618, rs552966, rs6592459, rs607446, rs607070, rs10713307, rs3018302, rs3750909, rs3018301, rs2511114, rs12270166, rs12270241, rs11606587, rs686340, rs548961, rs549032, rs575831, rs575878, rs577435, rs579320, rs495567, rs636946, rs493065, rs597513, rs598835, rs10683614, rs610004, rs610041, rs673478, rs670802, rs505041, rs2511116, rs628025, rs517837, rs615000, rs482013, rs693391, rs2511079, rs2250866, rs2508860, rs7483267, rs2511078, rs2508859, rs2508858, rs11235435, rs11235436, rs639435, rs12285624, position 112784 of SEQ ID NO: 4, rs624363, position 113614 of SEQ ID NO: 4, rs1053573, rs1053511, rs1063863, rs12137, rs4365081, rs4466868, rs3750911, rs510925, rs595062, rs1053443, rs542752, rs3897579, rs11235437, rs2508856, rs5792575, rs659513, rs10898820, rs2276397, rs3750908, rs3793938, rs602285, rs2276396, rs2276395, rs1806778, rs4073394, rs471547, rs606136, rs605241, rs686063, rs685749, rs533207, rs476753, position 191149 of SEQ ID NO: 4, rs11278712, and rs3831387. Polymorphic variants at the following positions in particular were associated with an increased risk of breast cancer: rs7949480, rs7121260, rs7128317, rs2276385, rs2276383, rs1892921, rs1541306, rs3814721, rs1892919, rs2852365, rs3750913, position 34941 in SEQ ID NO: 4, rs949325, position 35629 in SEQ ID NO: 4, rs2298456, rs1573500, rs4945411, rs10128658, rs2298457, rs3838779, rs10898814, rs7930142, rs7930544, rs7930722, rs1939247, rs1939243, rs4245463, rs4944258, rs7926751, rs1573502, rs7127865, rs1894003, rs4945430, rs1939242, rs10793016, rs4945434, rs678193, rs560777, rs676721, rs585228, rs675185, rs5792574, rs2511075, rs541022, rs1791544, rs642618, rs552966, rs607446, rs3018302, rs3018301, rs2511114, rs548961, rs579320, rs495567, rs493065, rs610004, rs610041, rs673478, rs670802, rs2511116, rs615000, rs482013, rs2511079, rs2250866, rs2508858, rs2276396, and rs11278712. At these positions in SEQ ID NO: 4, a thymine at position 3146, a guanine at position 6124, an adenine at position 6925, a guanine at position 7060, an adenine at position 7090, a cytosine at position 7696, a cytosine at position 13571, a guanine at position 14830, a cytosine at position 17733, a guanine at position 20776, a guanine at position 31726, a cytosine at position 34941, an adenine at position 35214, a guanine at position 35629, a thymine at position 36273, a cytosine at position 38348, a guanine at position 38608, a cytosine at position 39077, a thymine at position 39769, a thymine at position 40555, a thymine at position 42063, a cytosine at position 42574, a cytosine at position 42912, a cytosine at position 43042, a thymine at position 46079, a cytosine at position 48293, a cytosine at position 48771, a thymine at position 48951, a thymine at position 49972, a guanine at position 52986, a guanine at position 53339, a thymine at position 55834, a guanine at position 58703, a cytosine at position 59140, a guanine at position 60621, a cytosine at position 64645, a thymine at position 75831, a thymine at position 76160, a cytosine at position 76196, a cytosine at position 78847, a thymine at position 81130, a deletion at position 81452, a thymine at position 85815, a guanine at position 88614, a cytosine at position 90515, a cytosine at position 93751, an adenine at position 93775, a cytosine at position 94810, a thymine at position 96500, an adenine at position 97629, a thymine at position 97705, a guanine at position 99445, a guanine at position 100512, a cytosine at position 101046, an adenine at position 102487, a cytosine at position 103497, an adenine at position 103526, a cytosine at position 104662, a thymine at position 105226, a cytosine at position 107718, a thymine at position 108510, a cytosine at position 109520, a cytosine at position 109712, a thymine at position 110071, a cytosine at position 110963, a guanine at position 129463, and a “GTCAAC” at positions 34468-34472, in particular were associated with risk of breast cancer.

In the GALE locus, these included polymorphic variants at positions selected from the group consisting of rs627451, rs12082331, rs2267960, rs7519640, rs12086741, rs3934189, rs12135415, rs12088267, rs12084665, rs550850, rs557227, rs12137502, rs12118858, rs2294495, rs12401650, rs2235541, rs520713, rs550252, position 12668 of SEQ ID NO: 5, rs12093683, rs10917421, rs2744873, rs12091764, position 15761 of SEQ ID NO: 5, rs2076345, rs2076346, rs2473379, rs6679300, rs11811395, rs8179467, rs8179468, rs10917423, rs7536699, rs3215497, rs1000213, rs1000212, rs100211, rs1045017, rs12059520, rs2143118, rs12126804, rs533276, rs12402174, rs2864110, rs11581873, rs12062449, rs12033190, rs10157566, rs486746, rs11802707, rs6668550, rs7410946, rs7410970, rs6657558, rs2143119, rs7514394, rs11591202, rs6673991, position 37720 of SEQ ID NO: 5, rs12034848, rs2502984, rs3831910, rs11379444, rs725119, rs10917425, rs12077788, rs12077830, rs2473380, rs2473381, rs11545132, rs1046924, rs4237, rs4375317, rs11584220, rs2232975, rs2502985, rs3835423, rs2232976, rs2232977, rs2232978, rs2232979, rs2232980, rs2232981, rs2232982, rs2232983, rs2232984, rs2232985, rs2232986, rs2745, rs2744, rs11546127, rs1049988, rs1803613, rs1803612, position 55821 of SEQ ID NO: 5, rs3209973, rs3180545, rs3180383, rs3177814, rs3180546, rs3180547, rs11805398, rs760941, rs3177813, rs3180548, rs3179864, rs3177812, rs3179863, rs6692104, rs3180549, rs3179862, rs3177811, rs3177810, rs3180382, rs11546126, rs7551384, rs12141511, rs2473382, rs12038166, rs6666908, rs12041159, rs12031592, rs1062598, rs11714, rs3203593, rs1042436, rs2076343, rs12131539, rs11583935, rs12140263, rs3835225, rs2473374, rs2256179, rs974698, rs1018396, rs719399, rs719400, rs2502986, rs6679378, rs2473375, rs2473376, rs12136455, rs2502987, rs11551767, rs11591205, rs12083298, rs12138604, rs12136042, rs12138606, rs12136045, rs12116596, rs12138610, rs12136069, rs12116617, rs7540056, rs12044452, rs2076344, rs11588187, rs6696932, rs12025531, rs6682888, rs2982390, rs2179394, rs2473377, rs2179395, rs2502978, rs2502979, rs6698535, rs2502980, rs6697805, rs6424114, rs5773058, rs4649114, rs6424115 and rs10554242. Polymorphic variants at the following positions in particular were associated with an increased risk of breast cancer: rs2294495, position 15761 of SEQ ID NO: 5, rs8179467, rs11591202, rs12034848, rs3831910, rs4237, rs2232982, rs12041159, rs2076343, rs1108396, rs12044452, rs2982390, rs2502980, rs6697805, and rs10554242. At these positions in SEQ ID NO: 5, a cytosine at position 9351, a thymine at position 15761, a thymine at position 18220, an adenine at position 35555, a thymine at position 38304, an adenine at position 39985, an adenine at position 47604, a thymine at position 53102, a guanine at position 61135, an adenine at position 62601, a thymine at position 67087, a guanine at position 77120, a cytosine at position 81305, a cytosine at position 83464, a cytosine at position 85471, and a deletion at positions 83916-83920, in particular were associated with risk of breast cancer.

In the DPF3 locus, these included polymorphic variants at positions selected from the group consisting of rs11846274, rs2526935, rs929328, rs3742840, rs9671739, rs8006208, rs8006230, rs8007836, rs8006889, rs8010823, rs8011029, rs10649920, rs2803956, rs2803957, rs2526934, rs9671558, rs2526933, rs2803959, rs2526932, rs2803960, rs12434675, rs4899435, rs2526931, rs2803961, rs2803962, rs12590634, rs11327712, rs2803963, rs11844834, rs11320415, rs8012752, rs11623878, rs11623926, rs12434442, rs2526930, rs12435306, rs10144028, rs4899436, rs7140593, rs2107687, rs991882, rs3327, rs2526928, rs7401336, rs2526926, rs994760, rs994759, rs994758, rs11848404, rs2526925, rs11844236, rs2526924, rs2803965, rs2526923, rs7152467, rs11623462, rs2803966, rs2526922, rs2526920, rs11848135, rs11629210, rs2803967, rs16455, rs757655, rs5809581, rs3057139, rs2247658, rs11625609, rs8005580, rs8010472, rs2526919, rs2803968, rs11623943, rs11623946, rs1557926, rs12434611, rs2526918, rs5809582, rs8008279, rs2803969, rs2526917, rs8008774, rs2803970, rs8010255, rs2997642, rs2803971, rs11313760, rs2803972, rs4903038, rs2526916, rs4903040, rs2803974, rs2803975, rs2803976, rs2803977, rs10139909, rs722945, rs917069, rs2803978, rs2332890, rs1989638, rs10679174, rs2526915, rs2526914, rs11158964, rs11158965, rs740081, rs740080, rs10140582, rs2526913, rs4899437, rs10145678, rs2107686, rs2107685, rs2107684, rs7154188, rs4903041, rs7159637, rs6574089, rs12589753, rs4899438, rs4903042, rs4903043, rs2803980, rs10483848, rs2240344, rs2286068, rs11339669, rs3854, rs11463, rs1383, rs1381, rs1377, rs11158966, rs11158967, rs11158968, rs11158969, rs11158970, rs11158971, rs11849782, rs10137616, rs757572, rs10140566, rs2023480, rs2526911, rs4903044, rs11845162, rs12147210, rs2997643, rs2997644, rs3814867, rs2286067, rs1060570, rs2332891, rs2021736, rs7160830, rs2526909, rs7140963, rs10143667, rs2803981, rs768840, rs5809587, rs2023479, rs4903045, rs4903046, rs5809588, rs10220555, rs2906132, rs11848482, rs10129954, rs2526921, rs4903047, rs11846357, rs10136385, rs12588242, rs11621517, rs2526910, rs2526941, rs2803944, rs12436770, rs10220279, rs10220280, rs11844112, rs12432455, rs8015338, rs3832969, rs9671972, rs9671975, rs7159986, rs11848220, rs5016369, rs1986423, rs2332909, rs8013337, rs11445680, rs4903048, rs12434793, rs2332910, rs4410017, rs4577019, rs2332911, rs4996747, rs10140820, rs12431623, rs6574090, rs8008750, rs11627420, rs10150185, rs10150218, rs9646164, rs6574091, rs2332912, rs7155359, rs2332913, rs4544184, rs4337224, rs2332914, rs4280167, rs4903049, rs3742837, rs10139937, rs12434292, rs11620671, rs10140184, rs2332915, rs2332916, rs8012759, rs10149272, rs11158973, rs4903050, rs10666445, rs2536143, rs10638231, rs12431636, rs8005904, rs11621058, rs10133224, rs12433185, rs740974, rs740973, rs740972, rs740971, rs4243642, rs4415952, rs2215589, rs4343211, rs2536142, rs11293340, rs10151620, rs2286840, rs8023089, rs8008009, rs11419089, rs10694676, rs2536141, rs765247, rs7147626, rs10132629, rs4903051, rs11158974, rs1035021, rs6574093,rs4899439, rs7155287, rs7154402, rs7154781, rs6574094, rs10132242, rs10146959, rs10147466,rs11626411, rs4899440, rs10144553, rs11627663, rs11627799, rs11851733, rs10140317, rs8007375, rs4899441, rs11158975, rs11419607, rs2109793, rs8023156, rs10140579, rs7148199, rs7146916, rs1990439, rs6574095, rs10140037, rs8007249, rs1990438, rs1990437, rs10146484, rs11627333, rs7155802, rs2109792, rs11429363, rs8004210, rs2098194, rs2109791, rs11381729, rs1160094, rs11628051, rs10136771, rs10139903, rs4903053, rs3742836, rs3742835, rs8003233, rs11621588, rs740981, rs8019492, rs8019644, rs8019646, rs8019653, rs11450794, rs11348193, rs11450793, rs11282735, rs2191823, rs12232220, rs11158976, rs4903054,rs11846755, rs12431988, rs12100526,rs11622568, rs9671230, rs12147487, rs4903055, rs12147515, rs11625092, rs4899442, rs2052146, rs10220553, rs10220565, rs2332917, rs7158910, rs8015749, rs8006812, rs8008338, rs2052145, rs2052144, rs10146692, rs11627305, rs2079989, rs8018881, rs4561387, rs11622025, rs740980, rs12433250, rs5809590, rs758915, rs758914, rs7154229, rs12437188, rs7159108, rs4903058, rs4899443, rs11848907, rs12432148, rs12435078, rs6574096, rs11158977, rs11629434, rs4378563, rs2098195, rs4633653, rs7148350, rs740979, rs740978, rs740977, rs740976, rs4903059, rs4903060, rs11852056, rs9635257, rs9635258, rs9635259, rs4899444, rs12435382, rs12435412, rs11627381, rs2052143, rs2052142, rs2052141, rs8021035, rs11158978, rs11621216, rs10130873, rs11621358, rs758913, rs11622602, rs10136931, rs740975, rs747987, rs11623819, rs12434445, rs8003375, rs10143596, rs12435321, rs12435401, rs8010609, rs11158979, rs11158980, rs11627524, rs1126160, rs10136074, rs4903061, rs10665398, rs6574097, rs9671654, rs10140024, rs10142421, rs10142593, rs11423372, rs5809592, rs2332918, rs2332919, rs8007348, rs1990443, rs8007690, rs3937455, rs8012823, rs4899445, rs4903062, rs973963, rs10149962, rs8003449, rs11620775, rs8009089, rs8008967, rs12050132, rs10133018, rs1990442, rs1990441, rs8015833, rs1990440, rs4903063, rs10139810, rs4903064, rs2159715, rs2109795, rs2159714, rs11623107, rs7152005, rs7152489, rs4479167, rs4606655, rs4606656, rs4489943, rs7142642, rs8006233, rs8007559, rs8006156, rs1468662, rs11627048, rs6574098, rs12433760, rs12433780, rs12434506, rs12434673, rs12431400, rs12431384, rs4903067, rs2332920, rs2215591, rs12433064, rs12436263, rs12433097, rs12433184, rs8015900,rs11296981, rs11344025, rs11464204, rs11441172, rs11381807, rs10873251, rs2109794, rs10873252, rs4520784, rs2877820, rs2877821, rs4569195, rs11628905, rs2191822, rs2191821, rs11464689,rs12431959, rs4903068, rs1544579, rs7155879, rs12433603, rs11351432, rs11448438, rs4243643, rs4140952, rs8005372, rs8004109, rs8008398, rs8009692, rs2332921, rs12050320, rs12050323, rs8010316, rs7154293, rs12147969, rs2215590, rs4265749, rs1004552, rs4307892, rs12431973, rs12431975, rs767874, rs11628597, rs10129371, rs4903069, rs4903070, rs4903071, rs11622224, rs10459455, rs9323577, rs10624648, rs3058953, rs11158981, rs11158982, rs1158983, rs1860750,rs1860749, rs1860748, rs1860747, rs11419679, rs8011777, rs6574099, rs8010957, rs11626895, rs10151431, rs4903072, rs4589489, rs9671327, rs4899446, rs10144501, rs5809594, rs720317, rs8014316, rs 11158984, rs6420906, rs8004055, rs12434800, rs4243644, rs4899447, rs4903073, rs11848634, rs4899448, rs10131564, rs10134150, rs763388, rs11845938, rs11845946, rs11845915, rs10137463, rs6574100, rs7140344, rs10646889, rs7146287, rs5809595, rs1035099, rs7159877, rs7142579, rs12431762, rs12431764, rs10647999, rs6574101, rs6574102, rs1861162, rs12432972, rs10146134, rs10146616, rs10146769, rs8018936, rs10483849, rs2160137, rs2192595, rs2192594, rs11271695, rs10747301, rs12050363, rs12050368, rs12432949, rs7401375, rs8019682, rs8021248, rs11158985, rs10483850, rs8010875, rs11626844, rs982972, rs7161198, rs7160347, rs8007139, rs8014483, rs12437072, rs8004767, rs12588830, rs7158449, rs6574103, rs8020478, rs4903077, rs8012359, rs4899449, rs10690289, rs6574104, rs2110552, rs8008256, rs759283, rs4999177, rs2333006, rs8006178, rs11158986, rs7150625, rs8007768, rs8007058, position 284742 of SEQ ID NO: 6, rs2215901, rs3814866, rs3814864, and rs10605948. Polymorphic variants at the following positions in particular were associated with an increased risk of breast cancer: rs3327, rs2526926, rs2526914, rs2526913, rs2526921, rs7159986, rs2332916, rs10132242, rs11627663, rs4899441, rs7148199, rs1990437, rs8004210, rs8019653, rs12232220, rs4899442, rs2332917, rs8015749, rs12437188, rs12432148, rs4378563, rs740978, rs740976, rs4903059, rs4903060, rs9635259, rs2052142, rs758913, rs747987, rs11423372, rs5809592, rs8007348, rs4899445, rs4903062, rs1990440, rs4479167, rs4606655, rs4606656, rs4489943, rs8007559, rs8006156, rs12431400, rs12431384, rs12433097, rs12433184, rs2877821, rs4569195, rs11628905, rs2191822, rs12433603, rs11448438, rs8009692, rs4265749, rs4307892, rs12431975, rs4903071, rs1860749, rs1860748, rs8010957, rs6420906, rs12434800, rs7142579, rs8010875, rs8004767, rs4899449, and rs10605948. At these positions in SEQ ID NO: 6, a thymine at position 17641, a thymine at position 19527, a thymine at position 40889, an adenine at position 44877, a cytosine at position 75061, a thymine at position 86006, a cytosine at position 106988, a guanine at position 122783, a cytosine at position 125329, an adenine at position 127544, a cytosine at position 131734, a cytosine at position 137499, an adenine at position 139319, a guanine at position 147479, a guanine at position 150798, a guanine at position 160835, a guanine at position 163447, a guanine at position 164377, a cytosine at position 171972, a guanine at position 173467, a thymine at position 177780, a guanine at position 179608, a thymine at position 179808, a cytosine at position 180041, a guanine at position 181420, a guanine at position 182248, a guanine at position 185633, a guanine at position 188352, a thymine at position 189444, a cytosine at position 196415, a cytosine at position 196417, an adenine at position 196621, a guanine at position 197612, a cytosine at position 197854, a guanine at position 201045, a cytosine at position 205135, a thymine at position 205269, a thymine at position 205323, an adenine at position 205364, an adenine at position 206786, a guanine at position 206796, a thymine at position 209750, a guanine at position 209817, a guanine at position 211659, a thymine at position 211757, an adenine at position 213029, a guanine at position 213095, a cytosine at position 214247, a cytosine at position 214807, a guanine at position 217143, a guanine at position 217725, a thymine at position 218890, a thymine at position 222234, an adenine at position 222458, an adenine at position 222749, a thymine at position 227815, an adenine at position 233670, a cytosine at position 233702, a guanine at position 237317, a thymine at position 242360, an adenine at position 243355, an adenine at position 251858, a guanine at position 267130, a cytosine at position 273399, a guanine at position 277166, and a deletion at positions 250079-250082, in particular were associated with risk of breast cancer.

In the LOC145197 locus, these included polymorphic variants at positions selected from the group consisting of rs2895907, rs2401019, rs2401020, rs2401021, rs1999611, rs1999610, rs1999609, rs2275016, rs2275015, rs3818772, rs10149458, rs7142383, rs11343023, rs11160641, rs12586334, rs1142208, rs7493230, rs5811036, rs10151926, rs12435354, rs7151007, rs11160642, rs11160643, rs11626687, rs4906112, rs8016071, rs11623698, rs7401628, rs8015995, rs10873526, rs11160644, rs10134252, rs1959030, rs9707396, rs2401022, rs4622448, rs10141019, rs10141096, rs11621063, rs12434632, rs12100901, rs12434655, rs9944103, rs7153189, rs7158860, rs12437080, rs11851768, rs12431943, rs4309341, rs4329858, rs10690268, rs2401023, rs1054745, rs8019453, rs12431510, rs7141067, rs12431547, rs7142316, rs1959029, rs1959028, rs11376292, rs11376755, rs7145420, rs8013347, rs2144070, rs11850135, rs11850138, rs1955617, rs11621899, rs4900508, rs4906116, rs4906117, rs2235972, rs2235973, rs2235974, rs2235975, rs7149416, rs11850809, rs10140476, rs12437309, rs6575852, rs4906118, rs11428825, rs11450744, rs1999612, rs1889368,rs1889367, rs1889366, rs3077388, rs1889365, rs1955615, rs1569807, rs2064491, rs2064492, rs761532, rs1955616, rs2144065, rs2144066, rs2895908, rs2401043, rs1007904, rs3891113, rs3891114, rs730812, rs1884068, and rs1884069. Polymorphic variants at the following positions in particular were associated with an increased risk of breast cancer: rs12437080, rs12431943, rs1054745, rs8019453, rs12431547, rs1959029, rs1959028, rs8013347, rs2235972, rs2235973, rs2235974, and rs2895908. At these positions in SEQ ID NO: 7, a guanine at position 41414, an adenine at position 42821, an adenine at position 46344, a cytosine at position 46669, an adenine at position 47293, a guanine at position 49821, a thymine at position 49875, a thymine at position 51912, a cytosine at position 55799, a cytosine at position 55995, an adenine at position 56042, and an adenine at position 75221, in particular were associated with risk of breast cancer.

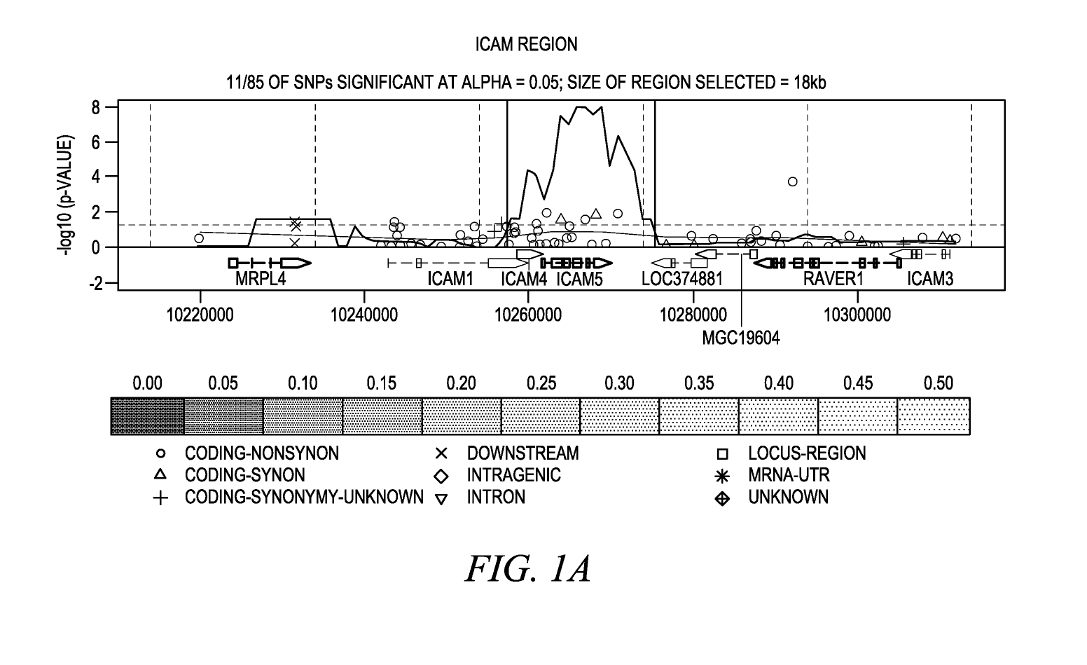

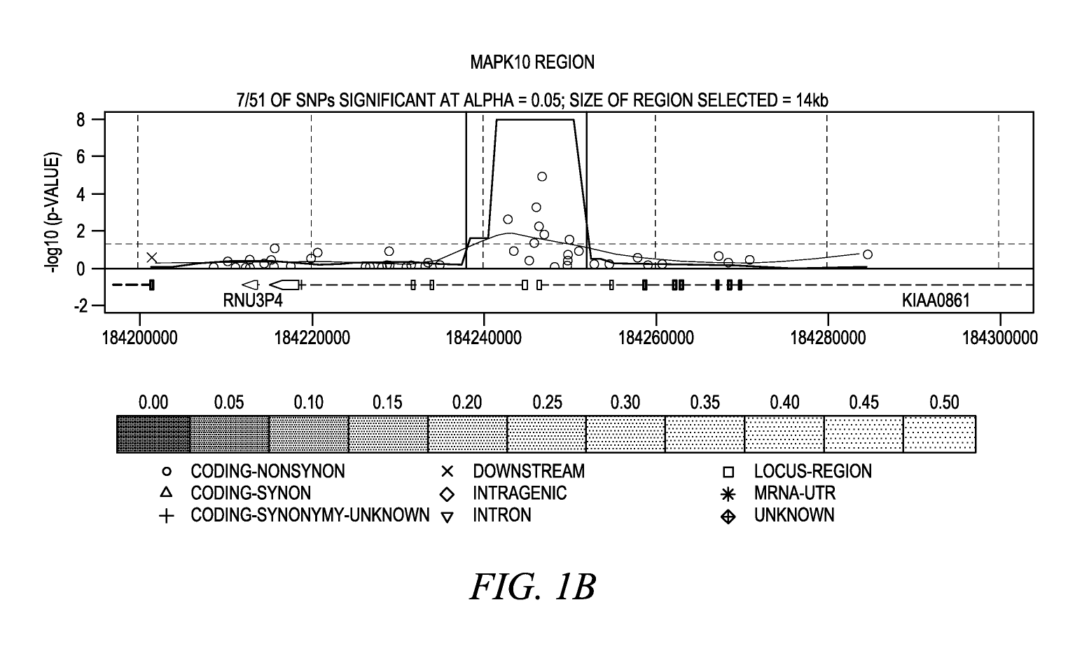

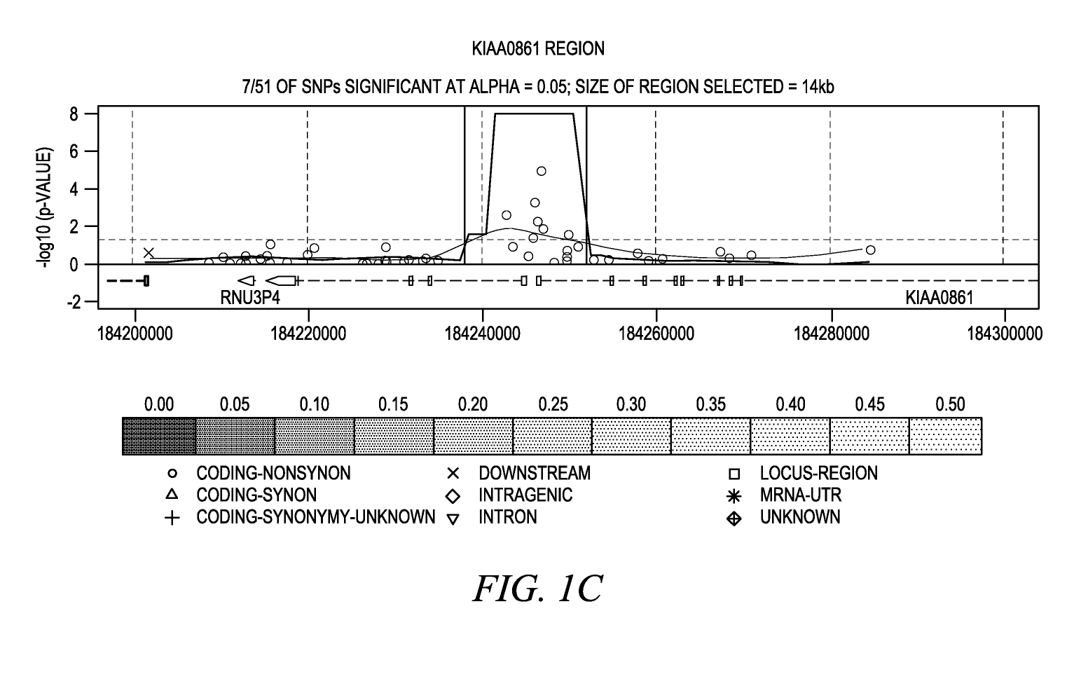

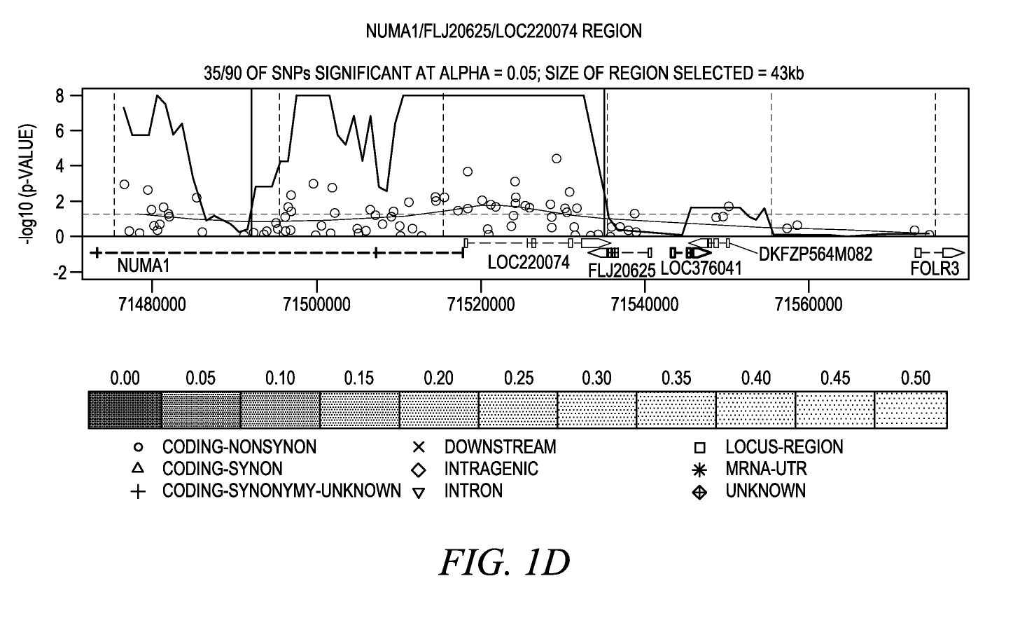

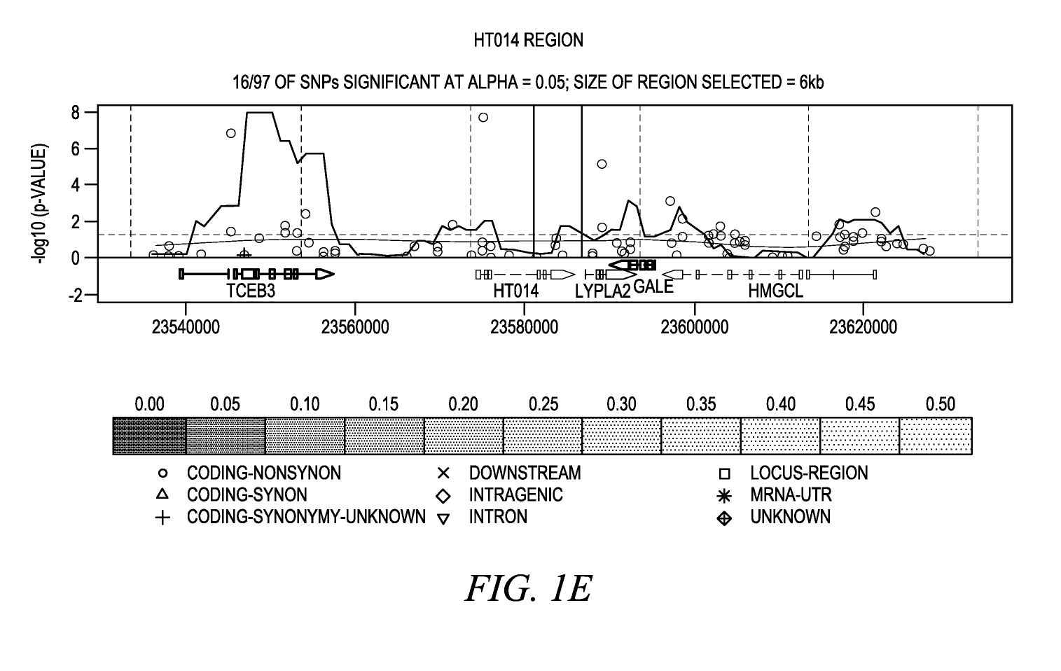

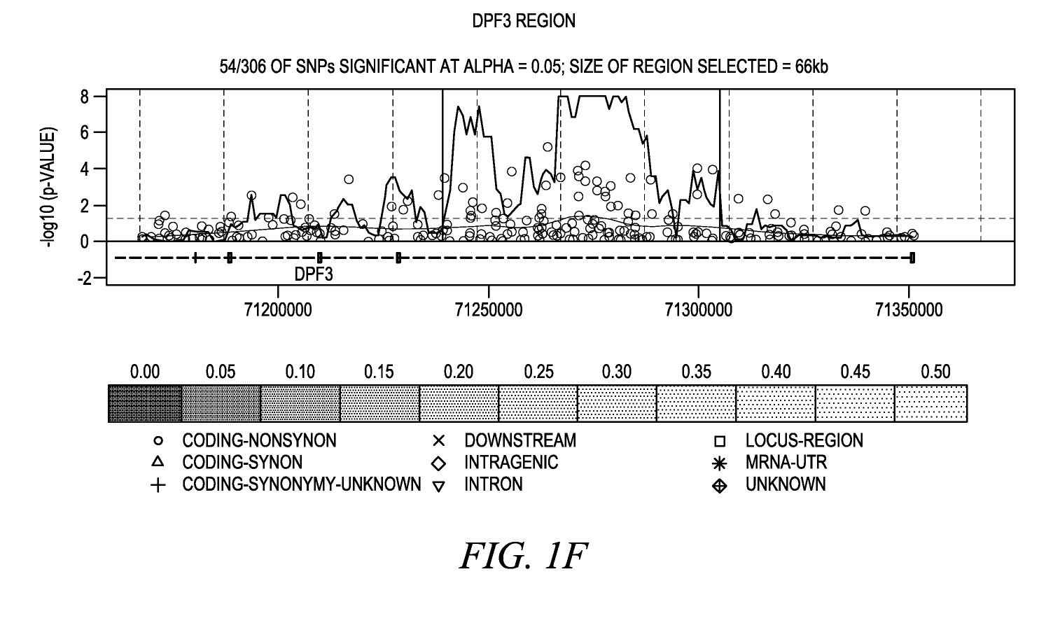

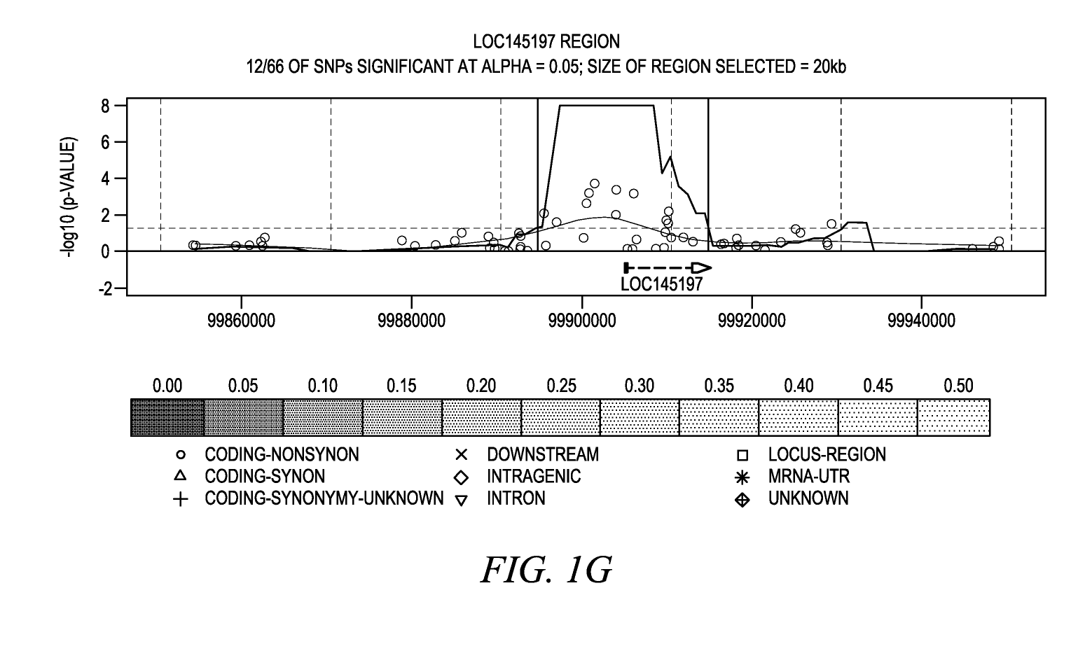

Based in part upon analyses summarized in FIGS. 1A-1G, regions with significant association have been identified in ICAM, MAPK10, KIAA0861, NUMA1, GALE, DPF3 and LOC145197 regions associated with increased risk of breast cancer. Any polymorphic variants associated with an increased risk of breast cancer in a region of significant association can be utilized for embodiments described herein. The following reports such a region, where “begin” and “end” designate the boundaries of the region according to chromosome positions within NCBI's Genome Build 34. For example, a region spanning positions 184215647 to 184249849 in the KIAA0861 locus was significantly associated with an increased risk of breast cancer.

| Rs # | Region | Region of Association | | Distance |
|---|---|---|---|---|
| 4237 | HT014 region | 23545351 | 23622196 | 76845 |
| 2001449 | KIAA0861 | 184215647 | 184249849 | 34202 |
| 1541998 | MAPK10 | 87386018 | 87442874 | 56856 |
| 673478 | NUMA1 | 71423896 | 71531713 | 107817 |
| 1054745 | LOC145197 | 99895564 | 99929371 | 33807 |
| 1990440 | DPF3 | 71078327 | 71343316 | 264989 |
| 11549918 | ICAM1, 4, 5 | 10231542 | 10268169 | 36627 |

The embodiments described hereafter may be directed to any of the polymorphic variants described herein. Sometimes, embodiments are directed to any of the polymorphic variants described herein with the proviso that the embodiments are not directed to a polymorphic variant at one or more positions selected from the group consisting of rs11549918, rs1541998, rs2001449, rs673478, rs4237, rs1990440 and rs1054745.

Additional Polymorphic Variants Associated with Breast Cancer

Also provided is a method for identifying polymorphic variants proximal to an incident, founder polymorphic variant associated with breast cancer. Thus, featured herein are methods for identifying a polymorphic variation associated with breast cancer that is proximal to an incident polymorphic variation associated with breast cancer, which comprises identifying a polymorphic variant proximal to the incident polymorphic variant associated with breast cancer, where the incident polymorphic variant is in a nucleotide sequence set forth in SEQ ID NO: 1-7. The nucleotide sequence often comprises a polynucleotide sequence selected from the group consisting of (a) a nucleotide sequence set forth in SEQ ID NO: 1-7; (b) a nucleotide sequence which encodes a polypeptide having an amino acid sequence encoded by a nucleotide sequence in SEQ ID NO: 1-7; (c) a nucleotide sequence which encodes a polypeptide that is 90% or more identical to an amino acid sequence encoded by a nucleotide sequence in SEQ ID NO: 1-7 or a nucleotide sequence about 90% or more identical to the nucleotide sequence set forth in SEQ ID NO: 1-7; and (d) a fragment of a nucleotide sequence of (a), (b), or (c), often a fragment that includes a polymorphic site associated with breast cancer. The presence or absence of an association of the proximal polymorphic variant with breast cancer then is determined using a known association method, such as a method described in the Examples hereafter. In an embodiment, the incident polymorphic variant is described in SEQ ID NO: 1-7. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic acid samples. Thus, multiple polymorphic variants proximal to an incident polymorphic variant are associated with breast cancer using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with breast cancer are identified iteratively. For example, a first proximal polymorphic variant is associated with breast cancer using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with breast cancer is determined.

The methods described herein are useful for identifying or discovering additional polymorphic variants that may be used to further characterize a gene, region or loci associated with a condition, a disease (e.g., breast cancer), or a disorder. For example, allelotyping or genotyping data from the additional polymorphic variants may be used to identify a functional mutation or a region of linkage disequilibrium.

In certain embodiments, polymorphic variants identified or discovered within a region comprising the first polymorphic variant associated with breast cancer are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with breast cancer, and such information can be used in prognosis methods described herein.

Isolated ICAM, MAPK10, KIAA0861, NUMA1, GALE, DPF3 and LOC145197 Nucleic Acids

Featured herein are isolated ICAM, MAPK10, KIAA0861, NUMA1, GALE, DPF3 or LOC145197 nucleic acids, which include the nucleic acid having the nucleotide sequence of SEQ ID NO: 1, 2, 3, 4, 5, 6, 7, 8, 9, 10 or 11, nucleic acid variants, and substantially identical nucleic acids of the foregoing. Nucleotide sequences of the ICAM, MAPK10, KIAA0861, NUMA1, GALE, DPF3 or LOC145197 nucleic acids sometimes are referred to herein as “ICAM, MAPK10, KIAA0861, NUMA1, GALE, DPF3 or LOC145197 nucleotide sequences.” An “ICAM, MAPK10, KIAA0861, NUMA1, GALE, DPF3 or LOC145197 nucleic acid variant” refers to one allele that may have one or more different polymorphic variations as compared to another allele in another subject or the same subject. A polymorphic variation in the ICAM, MAPK10, KIAA0861, NUMA1, GALE, DPF3 or LOC145197 nucleic acid variant may be represented on one or both strands in a double-stranded nucleic acid or on one chromosomal complement (heterozygous) or both chromosomal complements (homozygous).

As used herein, the term “nucleic acid” includes DNA molecules (e.g., a complementary DNA (cDNA) and genomic DNA (gDNA)) and RNA molecules (e.g., mRNA, rRNA, and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “ICAM, MAPK10, KIAA0861, NUMA1, GALE, DPF3 or LOC145197 gene” refers to a nucleotide sequence that encodes a ICAM, MAPK10, KIAA0861, NUMA1, GALE, DPF3 or LOC145197polypeptide.

Also included herein are nucleic acid fragments. These fragments typically are a nucleotide sequence identical to a nucleotide sequence in SEQ ID NO: 1-17, a nucleotide sequence substantially identical to a nucleotide sequence in SEQ ID NO: 1-17, or a nucleotide sequence that is complementary to the foregoing. The nucleic acid fragment may be identical, substantially identical or homologous to a nucleotide sequence in an exon or an intron in SEQ ID NO: 1-7, and may encode a domain or part of a domain or motif of a ICAM, MAPK10, KIAA0861, NUMA1, GALE, DPF3 or LOC145197 polypeptide. Sometimes, the fragment will comprises the polymorphic variation described herein as being associated with breast cancer. The nucleic acid fragment sometimes is 50, 100, or 200 or fewer base pairs in length, and is sometimes about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3800, 4000, 5000, 6000, 7000, 8000, 9000, 10000, 15000, 20000, 30000, 40000, 50000, 60000, 70000, 80000, 90000, 100000, 110000, 120000, 130000, 140000, 150000 or 160000 base pairs in length. A nucleic acid fragment complementary to a nucleotide sequence identical or substantially identical to the nucleotide sequence of SEQ ID NO: 1-17 and hybridizes to such a nucleotide sequence under stringent conditions often is referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI 3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides also may be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

### Uses for Nucleic Acid Sequences

Nucleic acid coding sequences depicted in SEQ ID NO: 1-17 may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Ribozymes may be engineered hammerhead motif ribozyme molecules that specifically and efficiently catalyze endonucleolytic cleavage of RNA sequences corresponding to or complementary to the nucleotide sequences set forth in SEQ ID NO: 1-17. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including breast cancer, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide can be the cause of various disease states, such as breast cancer. In situ hybridizations using polynucleotide probes may be employed to predict problems related to breast cancer. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a ICAM, MAPK10, KIAA0861, NUMA1, GALE, DPF3 or LOC145197 nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a ICAM, MAPK10, KIAA0861, NUMA1, GALE, DPF3 or LOC145197 nucleic acid in a form suitable for expression of the nucleic acid in a host cell. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce ICAM, MAPK10, KIAA0861, NUMA1, GALE, DPF3 or LOC145197 polypeptides, including fusion polypeptides, encoded by ICAM, MAPK10, KIAA0861, NUMA1, GALE, DPF3 or LOC145197 nucleic acids.

Recombinant expression vectors can be designed for expression of ICAM, MAPK10, KIAA0861, NUMA1, GALE, DPF3 or LOC145197 polypeptides in prokaryotic or eukaryotic cells. For example, ICAM, MAPK10, KIAA0861, NUMA1, GALE, DPF3 or LOC145197 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for ICAM, MAPK10, KIAA0861, NUMA1, GALE, DPF3 or LOC145197 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the α-fetopolypeptide promoter (Campes & Tilghman, Genes Dev. 3: 537-546 (1989)).

A ICAM, MAPK10, KIAA0861, NUMA1, GALE, DPF3 or LOC145197 nucleic acid may also be cloned into an expression vector in an antisense orientation. Regulatory sequences (e.g., viral promoters and/or enhancers) operatively linked to a ICAM, MAPK10, KIAA0861, NUMA1, GALE, DPF3 or LOC145197 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub et al., Antisense RNA as a molecular tool for genetic analysis, Reviews—Trends in Genetics, Vol. 1(1) (1986).

Also provided herein are host cells that include a ICAM, MAPK10, KIAA0861, NUMA1, GALE, DPF3 or LOC145197 nucleic acid within a recombinant expression vector or ICAM, MAPK10, KIAA0861, NUMA1, GALE, DPF3 or LOC145197 nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a ICAM, MAPK10, KIAA0861, NUMA1, GALE, DPF3 or LOC145197 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a ICAM, MAPK10, KIAA0861, NUMA1, GALE, DPF3 or LOC145197 polypeptide. Accordingly, further provided are methods for producing a ICAM, MAPK10, KIAA0861, NUMA1, GALE, DPF3 or LOC145197 polypeptide using the host cells described herein. In one embodiment, the method includes culturing host cells into which a recombinant expression vector encoding a ICAM, MAPK10, KIAA0861, NUMA1, GALE, DPF3 or LOC145197 polypeptide has been introduced in a suitable medium such that a ICAM, MAPK10, KIAA0861, NUMA1, GALE, DPF3 or LOC145197 polypeptide is produced. In another embodiment, the method further includes isolating a ICAM, MAPK10, KIAA0861, NUMA1, GALE, DPF3 or LOC145197 polypeptide from the medium or the host cell.

Also provided are cells or purified preparations of cells which include a ICAM, MAPK10, KIAA0861, NUMA1, GALE, DPF3 or LOC145197 transgene, or which otherwise misexpress ICAM, MAPK10, KIAA0861, NUMA1, GALE, DPF3 or LOC145197 polypeptide. Cell preparations can consist of human or non-human cells, e.g., rodent cells, e.g., mouse or rat cells, rabbit cells, or pig cells. In certain embodiments, the cell or cells include a ICAM, MAPK10, KIAA0861, NUMA , GALE, DPF3 or LOC145197 transgene (e.g., a heterologous form of a ICAM, MAPK10, KIAA0861, NUMA1, GALE, DPF3 or LOC145197 such as a human gene expressed in non-human cells). The ICAM, MAPK10, KIAA0861, NUMA1, GALE, DPF3 or LOC145197 transgene can be misexpressed, e.g., overexpressed or underexpressed. In other embodiments, the cell or cells include a gene which misexpress an endogenous ICAM, MAPK10, KIAA0861, NUMA1, GALE, DPF3 or LOC145197 polypeptide (e.g., expression of a gene is disrupted, also known as a knockout). Such cells can serve as a model for studying disorders which are related to mutated or mis-expressed ICAM, MAPK10, KIAA0861, NUMA1, GALE, DPF3 or LOC145197 alleles or for use in drug screening. Also provided are human cells (e.g., a hematopoietic stem cells) transformed with a ICAM, MAPK10, KIAA0861, NUMA1, GALE, DPF3 or LOC145197 nucleic acid.

Also provided are cells or a purified preparation thereof (e.g., human cells) in which an endogenous ICAM, MAPK10, KIAA0861, NUMA1, GALE, DPF3 or LOC145197 nucleic acid is under the control of a regulatory sequence that does not normally control the expression of the endogenous ICAM, MAPK10, KIAA0861, NUMA1, GALE, DPF3 or LOC145197 gene. The expression characteristics of an endogenous gene within a cell (e.g., a cell line or microorganism) can be modified by inserting a heterologous DNA regulatory element into the genome of the cell such that the inserted regulatory element is operably linked to the endogenous ICAM, MAPK10, KIAA0861, NUMA1, GALE, DPF3 or LOC145197 gene. For example, an endogenous ICAM, MAPK10, KIAA0861, NUMA1, GALE, DPF3 or LOC145197 gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous ICAM, MAPK10, KIAA0861, NUMA1, GALE, DPF3 or LOC145197 polypeptide (e.g., expressed from a ICAM, MAPK10, KIAA0861, NUMA1, GALE, DPF3 or LOC145197 nucleic acid isolated from another organism) can be generated. Such animals are useful for studying the function and/or activity of a ICAM, MAPK10, KIAA0861, NUMA1, GALE, DPF3 or LOC145197 polypeptide and for identifying and/or evaluating modulators of ICAM, MAPK10, KIAA0861, NUMA1, GALE, DPF3 or LOC145197 nucleic acid and ICAM, MAPK10, KIAA0861, NUMA1, GALE, DPF3 or LOC145197 polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a ICAM, MAPK10, KIAA0861, NUMA1, GALE, DPF3 or LOC145197 transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous ICAM, MAPK10, KIAA0861, NUMA1, GALE, DPF3 or LOC145197 gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a ICAM, MAPK10, KIAA0861, NUMA1, GALE, DPF3 or LOC145197 transgene to direct expression of a ICAM, MAPK10, KIAA0861, NUMA1, GALE, DPF3 or LOC145197 polypeptide to particular cells. A transgenic founder animal can be identified based upon the presence of a ICAM, MAPK10, KIAA0861, NUMA1, GALE, DPF3 or LOC145197 transgene in its genome and/or expression of ICAM, MAPK10, KIAA0861, NUMA1, GALE, DPF3 or LOC145197 mRNA in tissues or cells of the animals. A transgenic founder animal can then be used to breed additional animals carrying the transgene. Moreover, transgenic animals carrying a transgene encoding a ICAM, MAPK10, KIAA0861, NUMA1, GALE, DPF3 or LOC145197 polypeptide can further be bred to other transgenic animals carrying other transgenes.

ICAM, MAPK10, KIAA0861, NUMA1, GALE, DPF3 or LOC145197 polypeptides can be expressed in transgenic animals or plants by introducing, for example, a nucleic acid encoding the polypeptide into the genome of an animal. In certain embodiments the nucleic acid is placed under the control of a tissue specific promoter, e.g., a milk or egg specific promoter, and recovered from the milk or eggs produced by the animal. Also included is a population of cells from a transgenic animal.

ICAM, MAPK10, KIAA0861, NUMA1, GALE, DPF3 and LOC145197 Polypeptides

Featured herein are isolated ICAM, MAPK10, KIAA0861, NUMA1, GALE, DPF3 or LOC145197 polypeptides, which include polypeptides having amino acid sequences set forth in SEQ ID NO: 18-27, and substantially identical polypeptides thereof. Such polypeptides sometimes are proteins or peptides. A ICAM, MAPK10, KIAA0861, NUMA1, GALE, DPF3 or LOC145197 polypeptide is a polypeptide encoded by a ICAM, MAPK10, KIAA0861, NUMA1, GALE, DPF3 or LOC145197 nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a ICAM, MAPK10, KIAA0861, NUMA1, GALE, DPF3 or LOC145197 polypeptide or ICAM, MAPK10, KIAA0861, NUMA1, GALE, DPF3 or LOC145197 polypeptide variant having less than about 30%, 20%, 10% and sometimes 5% (by dry weight), of non-ICAM, MAPK10, KIAA0861, NUMA1, GALE, DPF3 or LOC145197 polypeptide (also referred to herein as a “contaminating protein”), or of chemical precursors or non-ICAM, MAPK10, KIAA0861, NUMA1, GALE, DPF3 or LOC145197 chemicals. When the ICAM, MAPK10, KIAA0861, NUMA1, GALE, DPF3 or LOC145197 polypeptide or a biologically active portion thereof is recombinantly produced, it is also often substantially free of culture medium, specifically, where culture medium represents less than about 20%, sometimes less than about 10%, and often less than about 5% of the volume of the polypeptide preparation. Isolated or purified ICAM, MAPK10, KIAA0861, NUMA1, GALE, DPF3 or LOC145197 polypeptide preparations are sometimes 0.01 milligrams or more or 0.1 milligrams or more, and often 1.0 milligrams or more and 10 milligrams or more in dry weight. In specific embodiments, a polypeptide comprises leucine at amino acid position 359 in SEQ ID NO: 23, a leucine at amino acid position 378 in SEQ ID NO: 23, an alanine at amino acid position 857 in SEQ ID NO: 23, an alanine at position 902 in SEQ ID NO: 23 a proline at amino acid position 352 in SEQ ID NO: 20 and/or an alanine at amino acid position 348 in SEQ ID NO: 20. A polypeptide encoded by a nucleotide sequence in a NUMA1 region (e.g., NUMA1 polypeptide (e.g., SEQ ID NO: 24), FLJ20625 polypeptide (e.g., accession no. NM_{—}017907) and IL18BP polypeptide (e.g., accession nos. NM_{—}173042, NM_{—}173043 or NM_{—}173044)) sometimes includes one or more of the following amino acid variations: a leucine at a position corresponding to amino acid position 73 in an NM_{—}017907 polypeptide, a glutamine at a position corresponding to amino acid position 772 in SEQ ID NO: 24, a glutamate at a position corresponding to amino acid position 873 in SEQ ID NO: 24, a glycine at a position corresponding to amino acid position 794 in SEQ ID NO: 24, an arginine at a position corresponding to amino acid position 89 in an IL18BP polypeptide, an argenine at a position corresponding to amino acid position 119 in an IL18BP polypeptide, a cysteine at a position corresponding to amino acid position 195 in a IL18BP polypeptide, an alanine at a position corresponding to amino acid position 2049 in SEQ ID NO: 24, a methionine at a position corresponding to amino acid position 1825 in SEQ ID NO: 24, and a glycine at a position corresponding to amino acid position 978 in SEQ ID NO: 24. A polypeptide encoded by a nucleotide sequence in a GALE region (e.g., GALE (e.g., SEQ ID NO: 25), TCEB3 (e.g., accession no. NM_{—}003198) and HT014 (e.g., accession no. NM_{—}020362)) sometimes includes one or more of the following amino acid variations: a valine at a position corresponding to amino acid position 298 in a TCEB3 polypeptide, a valine at a position corresponding to amino acid position 490 in a TCEB3 polypeptide, a methionine at a position corresponding to amino acid position 119 in a TCEB3 polypeptide, a glycine at a position corresponding to amino acid position 227 in SEQ ID NO: 25, an aspartate at a position corresponding to amino acid position 231 in SEQ ID NO: 25, an asparagine at a position corresponding to amino acid position 268 in SEQ ID NO: 25, a valine at a position corresponding to amino acid position 283 in SEQ ID NO: 25, a leucine at a position corresponding to amino acid position 215 in SEQ ID NO: 25, a glycine at a position corresponding to amino acid position 235 in SEQ ID NO: 25, a glutamate at a position corresponding to amino acid position 331 in SEQ ID NO: 25, a serine at a position corresponding to amino acid position 191 in a HT014 polypeptide and a tyrosine at a position corresponding to amino acid position 141 in SEQ ID NO: 25.

In another aspect, featured herein are ICAM, MAPK10, KIAA0861, NUMA1, GALE, DPF3 or LOC145197 polypeptides and biologically active or antigenic fragments thereof that are useful as reagents or targets in assays applicable to prevention, treatment or diagnosis of breast cancer. In another embodiment, provided herein are ICAM, MAPK10, KIAA0861, NUMA1, GALE, DPF3 or LOC145197 polypeptides having a ICAM, MAPK10, KIAA0861, NUMA1, GALE, DPF3 or LOC145197 activity or activities.

Further included herein are ICAM, MAPK10, KIAA0861, NUMA1, GALE, DPF3 or LOC145197 polypeptide fragments. The polypeptide fragment may be a domain or part of a domain of a ICAM, MAPK10, KIAA0861, NUMA1, GALE, DPF3 or LOC145197 polypeptide. The polypeptide fragment is often 50 or fewer, 100 or fewer, or 200 or fewer amino acids in length, and is sometimes 300, 400, 500, 600, 700, or 900 or fewer amino acids in length. In certain embodiments, the polypeptide fragment comprises, consists essentially of, or consists of, at least 6 consecutive amino acids and not more than 1211 consecutive amino acids of SEQ ID NO: 18-27, or the polypeptide fragment comprises, consists essentially of, or consists of, at least 6 consecutive amino acids and not more than 543 consecutive amino acids of SEQ ID NO: 18-27.

ICAM, MAPK10, KIAA0861, NUMA1, GALE, DPF3 or LOC145197 polypeptides described herein can be used as immunogens to produce anti-ICAM, MAPK10, KIAA0861, NUMA1, GALE, DPF3 or LOC145197 antibodies in a subject, to purify ICAM, MAPK10, KIAA0861, NUMA1, GALE, DPF3 or LOC145197 ligands or binding partners, and in screening assays to identify molecules which inhibit or enhance the interaction of ICAM, MAPK10, KIAA0861, NUMA1, GALE, DPF3 or LOC145197 with a ICAM, MAPK10, KIAA0861, NUMA1, GALE, DPF3 or LOC145197 substrate. Full-length ICAM, MAPK10, KIAA0861, NUMA1, GALE, DPF3 or LOC145197 polypeptides and polynucleotides encoding the same may be specifically substituted for a ICAM, MAPK10, KIAA0861, NUMA1, GALE, DPF3 or LOC145197 polypeptide fragment or polynucleotide encoding the same in any embodiment described herein.

Substantially identical polypeptides may depart from the amino acid sequences set forth in SEQ ID NO: 18-27 in different manners. For example, conservative amino acid modifications may be introduced at one or more positions in the amino acid sequences of SEQ ID NO: 18-27. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a ICAM, MAPK10, KIAA0861, NUMA1, GALE, DPF3 or LOC145197 polypeptide, whereas altering an “essential” amino acid abolishes or substantially alters the biological function of a ICAM, MAPK10, KIAA0861, NUMA1, GALE, DPF3 or LOC145197 polypeptide. Amino acids that are conserved among ICAM, MAPK10, KIAA0861, NUMA1, GALE, DPF3 or LOC145197 polypeptides are typically essential amino acids.

Also, ICAM, MAPK10, KIAA0861, NUMA1, GALE, DPF3 or LOC145197 polypeptides and polypeptide variants may exist as chimeric or fusion polypeptides. As used herein, a ICAM, MAPK10, KIAA0861, NUMA1, GALE, DPF3 or LOC145197 “chimeric polypeptide” or “fusion polypeptide” includes a ICAM, MAPK10, KIAA0861, NUMA1, GALE, DPF3 or LOC145197 polypeptide linked to a non-ICAM, MAPK10, KIAA0861, NUMA1, GALE, DPF3 or LOC145197 polypeptide. A “non-ICAM, MAPK10, KIAA0861, NUMA1, GALE, DPF3 or LOC145197 polypeptide” refers to a polypeptide having an amino acid sequence corresponding to a polypeptide which is not substantially identical to the ICAM, MAPK10, KIAA0861, NUMA1, GALE, DPF3 or LOC145197 polypeptide, which includes, for example, a polypeptide that is different from the ICAM, MAPK10, KIAA0861, NUMA1, GALE, DPF3 or LOC145197 polypeptide and derived from the same or a different organism. The ICAM, MAPK10, KIAA0861, NUMA1, GALE, DPF3 or LOC145197 polypeptide in the fusion polypeptide can correspond to an entire or nearly entire ICAM, MAPK10, KIAA0861, NUMA1, GALE, DPF3 or LOC145197 polypeptide or a fragment thereof. The non-ICAM, MAPK10, KIAA0861, NUMA1, GALE, DPF3 or LOC145197 polypeptide can be fused to the N-terminus or C-terminus of the ICAM, MAPK10, KIAA0861, NUMA1, GALE, DPF3 or LOC145197 polypeptide.

Fusion polypeptides can include a moiety having high affinity for a ligand. For example, the fusion polypeptide can be a GST-ICAM, MAPK10, KIAA0861, NUMA1, GALE, DPF3 or LOC145197 fusion polypeptide in which the ICAM, MAPK10, KIAA0861, NUMA1, GALE, DPF3 or LOC145197 sequences are fused to the C-terminus of the GST sequences, or a polyhistidine-ICAM, MAPK10, KIAA0861, NUMA1, GALE, DPF3 or LOC145197 fusion polypeptide in which the ICAM, MAPK10, KIAA0861, NUMA1, GALE, DPF3 or LOC145197 polypeptide is fused at the N- or C-terminus to a string of histidine residues. Such fusion polypeptides can facilitate purification of recombinant ICAM, MAPK10, KIAA0861, NUMA1, GALE, DPF3 or LOC145197. Expression vectors are commercially available that already encode a fusion moiety (e.g., a GST polypeptide), and a ICAM, MAPK10, KIAA0861, NUMA1, GALE, DPF3 or LOC145197 nucleic acid can be cloned into an expression vector such that the fusion moiety is linked in-frame to the ICAM, MAPK10, KIAA0861, NUMA1, GALE, DPF3 or LOC145197 polypeptide. Further, the fusion polypeptide can be a ICAM, MAPK10, KIAA0861, NUMA1, GALE, DPF3 or LOC145197 polypeptide containing a heterologous signal sequence at its N-terminus. In certain host cells (e.g., mammalian host cells), expression, secretion, cellular internalization, and cellular localization of a ICAM, MAPK10, KIAA0861, NUMA1, GALE, DPF3 or LOC145197 polypeptide can be increased through use of a heterologous signal sequence. Fusion polypeptides can also include all or a part of a serum polypeptide (e.g., an IgG constant region or human serum albumin).

ICAM, MAPK10, KIAA0861, NUMA1, GALE, DPF3 or LOC145197 polypeptides or fragments thereof can be incorporated into pharmaceutical compositions and administered to a subject in vivo. Administration of these ICAM, MAPK10, KIAA0861, NUMA1, GALE, DPF3 or LOC145197 polypeptides can be used to affect the bioavailability of a ICAM, MAPK10, KIAA0861, NUMA1, GALE, DPF3 or LOC145197 substrate and may effectively increase or decrease ICAM, MAPK10, KIAA0861, NUMA1, GALE, DPF3 or LOC145197 biological activity in a cell or effectively supplement dysfunctional or hyperactive ICAM, MAPK10, KIAA0861, NUMA1, GALE, DPF3 or LOC145197 polypeptide. ICAM, MAPK10, KIAA0861, NUMA1, GALE, DPF3 or LOC145197 fusion polypeptides may be useful therapeutically for the treatment of disorders caused by, for example, (i) aberrant modification or mutation of a gene encoding a ICAM, MAPK10, KIAA0861, NUMA1, GALE, DPF3 or LOC145197 polypeptide; (ii) mis-regulation of the ICAM, MAPK10, KIAA0861, NUMA1, GALE, DPF3 or LOC145197 gene; and (iii) aberrant post-translational modification of a ICAM, MAPK10, KIAA0861, NUMA1, GALE, DPF3 or LOC145197 polypeptide. Also, ICAM, MAPK10, KIAA0861, NUMA1, GALE, DPF3 or LOC145197 polypeptides can be used as immunogens to produce anti-ICAM, MAPK10, KIAA0861, NUMA1, GALE, DPF3 or LOC145197 antibodies in a subject, to purify ICAM, MAPK10, KIAA0861, NUMA1, GALE, DPF3 or LOC145197 ligands or binding partners, and in screening assays to identify molecules which inhibit or enhance the interaction of ICAM, MAPK10, KIAA0861, NUMA1, GALE, DPF3 or LOC145197 with a ICAM, MAPK10, KIAA0861, NUMA1, GALE, DPF3 or LOC145197 substrate.

In addition, polypeptides can be chemically synthesized using techniques known in the art (See, e.g., Creighton, 1983 Proteins. New York, N.Y.: W.H. Freeman and Company; and Hunkapiller et al., (1984) Nature July 12-18;310(5973): 105-11). For example, a relative short polypeptide fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Also included are polypeptide fragments which are differentially modified during or after translation, e.g., by glycosylation, acetylation, phosphorylation, amidation, derivatization by known protecting/blocking groups, proteolytic cleavage, linkage to an antibody molecule or other cellular ligand, and the like. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; and the like.

Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label, such as an enzymatic, fluorescent, isotopic or affinity label to allow for detection and isolation of the polypeptide.

Also provided are chemically modified polypeptide derivatives that may provide additional advantages such as increased solubility, stability and circulating time of the polypeptide, or decreased immunogenicity. See U.S. Pat. No. 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., EP 0 401 384, herein incorporated by reference (coupling PEG to G-CSF), see also Malik et al. (1992) Exp Hematol. September; 20(8): 1028-35, reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. A polymer sometimes is attached at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical Nucleic Acids and Polypeptides

Nucleotide sequences and polypeptide sequences that are substantially identical to a ICAM, MAPK10, KIAA0861, NUMA1, GALE, DPF3 or LOC145197 nucleotide sequence and the ICAM, MAPK10, KIAA0861, NUMA1, GALE, DPF3 or LOC145197 polypeptide sequences encoded by those nucleotide sequences are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) or more identical to the nucleotide sequences in SEQ ID NO: 1-17 or the encoded ICAM, MAPK10, KIAA0861, NUMA1, GALE, DPF3 or LOC145197 polypeptide amino acid sequences. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available on the World Wide Web at the URL “gcg.com”), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available on the World Wide Web at the URL “gcg.com”), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to a ICAM, MAPK10, KIAA0861, NUMA1, GALE, DPF3 or LOC145197 nucleotide sequence is one that has a different nucleotide sequence but still encodes the same polypeptide sequence encoded by the ICAM, MAPK10, KIAA0861, NUMA1, GALE, DPF3 or LOC145197 nucleotide sequence. Another example is a nucleotide sequence that encodes a polypeptide having a polypeptide sequence that is more than 70% or more identical to, sometimes 75% or more, 80% or more, or 85% or more identical to, and often 90% or more and 95% or more identical to a polypeptide sequence encoded by a ICAM, MAPK10, KIAA0861, NUMA1, GALE, DPF3 or LOC145197 nucleotide sequence.

ICAM, MAPK10, KIAA0861, NUMA1, GALE, DPF3 or LOC145197 nucleotide sequences and ICAM, MAPK10, KIAA0861, NUMA1, GALE, DPF3 or LOC145197 amino acid sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences from SEQ ID NO: 1-17. BLAST polypeptide searches can be performed with the XBLAST program, score=50, wordlength=3 to obtain amino acid sequences homologous to polypeptides encoded by a ICAM, MAPK10, KIAA0861, NUMA1, GALE, DPF3 or LOC145197 nucleotide sequence. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (available on the World Wide Web at the URL “ncbi.nlm.nih.gov”).

A nucleic acid that is substantially identical to a ICAM, MAPK10, KIAA0861, NUMA1, GALE, DPF3 or LOC145197 nucleotide sequence may include polymorphic sites at positions equivalent to those described herein when the sequences are aligned. For example, using the alignment procedures described herein, SNPs in a sequence substantially identical to a sequence in SEQ ID NO: 1-17 can be identified at nucleotide positions that match (i.e., align) with nucleotides at SNP positions in the nucleotide sequence of SEQ ID NO: 1-17. Also, where a polymorphic variation results in an insertion or deletion, insertion or deletion of a nucleotide sequence from a reference sequence can change the relative positions of other polymorphic sites in the nucleotide sequence.

Substantially identical nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50% or more, about 55% or more, often about 70-75% or more, more often about 80-85% or more, and typically about 90-95% or more identical to the amino acid sequences of target polypeptides or a fragment thereof. Such nucleic acid molecules readily can be identified as being able to hybridize under stringent conditions to a nucleotide sequence in SEQ ID NO: 1-17 or a fragment thereof. Nucleic acid molecules corresponding to orthologs, homologs, and allelic variants of a nucleotide sequence in SEQ ID NO: 1-17 can be identified by mapping the sequence to the same chromosome or locus as the nucleotide sequence in SEQ ID NO: 1-17.

Also, substantially identical nucleotide sequences may include codons that are altered with respect to the naturally occurring sequence for enhancing expression of a target polypeptide in a particular expression system. For example, the nucleic acid can be one in which one or more codons are altered, and often 10% or more or 20% or more of the codons are altered for optimized expression in bacteria (e.g., E. coli), yeast (e.g., S. cervesiae), human (e.g., 293 cells), insect, or rodent (e.g., hamster) cells.

Methods for Identifying Subjects at Risk of Breast Cancer and Breast Cancer Risk in a Subject

Methods for prognosing and diagnosing breast cancer in subjects are provided herein. These methods include detecting the presence or absence of one or more polymorphic variations associated with breast cancer in a nucleotide sequence set forth in SEQ ID NO: 1-7, or substantially identical sequence thereof, in a sample from a subject, where the presence of a polymorphic variant is indicative of a risk of breast cancer.

Thus, featured herein is a method for detecting a subject at risk of breast cancer or the risk of breast cancer in a subject, which comprises detecting the presence or absence of a polymorphic variation associated with breast cancer at a polymorphic site in a nucleic acid sample from a subject, where the nucleotide sequence comprises a polynucleotide sequence selected from the group consisting of: (a) a nucleotide sequence set forth in SEQ ID NO: 1-7; (b) a nucleotide sequence which encodes a polypeptide having an amino acid sequence encoded by a nucleotide sequence in SEQ ID NO: 1-7; (c) a nucleotide sequence which encodes a polypeptide that is 90% or more identical to an amino acid sequence encoded by a nucleotide sequence in SEQ ID NO: 1-7 or a nucleotide sequence about 90% or more identical to the nucleotide sequence set forth in SEQ ID NO: 1-7; and (d) a fragment of a nucleotide sequence of (a), (b), or (c), often a fragment that includes a polymorphic site associated with breast cancer; whereby the presence of the polymorphic variation is indicative of a risk of breast cancer in the subject. In certain embodiments, the polymorphic variant is detected at a position described herein. The term “SEQ ID NO: 1-7” as used herein refers to one or more of SEQ ID NO: 1, 2, 3, 4, 5, 6 or 7.

In certain embodiments, determining the presence of a combination of two or more polymorphic variants associated with breast cancer in one or more genetic loci (e.g., one or more genes) of the sample is determined to identify, quantify and/or estimate, risk of breast cancer. The risk often is the probability of having or developing breast cancer. The risk sometimes is expressed as a relative risk with respect to a population average risk of breast cancer, and sometimes is expressed as a relative risk with respect to the lowest risk group. Such relative risk assessments often are based upon penetrance values determined by statistical methods (see e.g., statistical analysis Example 9), and are particularly useful to clinicians and insurance companies for assessing risk of breast cancer (e.g., a clinician can target appropriate detection, prevention and therapeutic regimens to a patient after determining the patient's risk of breast cancer, and an insurance company can fine tune actuarial tables based upon population genotype assessments of breast cancer risk). Risk of breast cancer sometimes is expressed as an odds ratio, which is the odds of a particular person having a genotype has or will develop breast cancer with respect to another genotype group (e.g., the most disease protective genotype or population average). In related embodiments, the determination is utilized to identify a subject at risk of breast cancer. In an embodiment, two or more polymorphic variations are detected in two or more regions in human genomic DNA associated with increased risk of breast cancer, such as regions selected from the group of loci consisting of ICAM, MAPK10, KIAA0861, NUMA1, GALE, DPF3 and LOC145197, for example. In certain embodiments, 3 or more, or4, 5, 6, 7, 8, 9, 10, 11, 12, 13, 14, 15, 16, 17, 18, 19 or 20 or more polymorphic variants are detected in the sample. In specific embodiments, polymorphic variants are detected in ICAM, MAPK10, KIAA0861, NUMA1, GALE, DPF3 and LOC145197 loci, such as at positions 44247 in SEQ ID NO: 1 (ICAM), position 36424 in SEQ ID NO: 2 (MAPK10), position 48563 in SEQ ID NO: 3 (KIAA0861), position 49002 in SEQ ID NO: 4 (NUMA1) and position 174 in SEQ ID NO: 5 (GALE), for example. In certain embodiments, polymorphic variants are detected at other genetic loci (e.g., the polymorphic variants can be detected in ICAM, MAPK10, KIAA0861, NUMA1, GALE, DPF3 and/or LOC145197 in addition to other loci or only in other loci), where the other loci include but are not limited to RAD21, KLF12, SPUVE, GRIN3A, PFTK1, SERPINA5, LOC115209, HRMT1L3, DLG1, KIAA0783, DPF3, CENPC1, GP6, LAMA4, CHCB/C20ORF154, LOC338749, and TTN/LOC351327, which are described in patent applications Ser. Nos. 10/723,670, 10/723,518, 10/722,939 and 10/723,683, having attorney docket numbers 524592006700, 524592006800, 524592007100 and 524592007200, respectively, and any others disclosed in patent application Nos. 60/429,136 (filed Nov. 25, 2002) and 60/490,234 (filed Jul. 24, 2003).

A risk of developing aggressive forms of breast cancer likely to metastasize or invade surrounding tissues (e.g., Stage IIIA, IIIB, and IV breast cancers), and subjects at risk of developing aggressive forms of breast cancer also may be identified by the methods described herein. These methods include collecting phenotype information from subjects having breast cancer, which includes the stage of progression of the breast cancer, and performing a secondary phenotype analysis to detect the presence or absence of one or more polymorphic variations associated with a particular stage form of breast cancer. Thus, detecting the presence or absence of one or more polymorphic variations in a ICAM, MAPK10, KIAA0861, NUMA1, GALE, DPF3 or LOC145197 nucleotide sequence associated with a late stage form of breast cancer often is prognostic and/or diagnostic of an aggressive form of the cancer.

Results from prognostic tests may be combined with other test results to diagnose breast cancer. For example, prognostic results may be gathered, a patient sample may be ordered based on a determined predisposition to breast cancer, the patient sample is analyzed, and the results of the analysis may be utilized to diagnose breast cancer. Also breast cancer diagnostic methods can be developed from studies used to generate prognostic/diagnostic methods in which populations are stratified into subpopulations having different progressions of breast cancer. In another embodiment, prognostic results may be gathered; a patient's risk factors for developing breast cancer analyzed (e.g., age, race, family history, age of first menstrual cycle, age at birth of first child); and a patient sample may be ordered based on a determined predisposition to breast cancer. In an alternative embodiment, the results from predisposition analyses described herein may be combined with other test results indicative of breast cancer, which were previously, concurrently, or subsequently gathered with respect to the predisposition testing. In these embodiments, the combination of the prognostic test results with other test results can be probative of breast cancer, and the combination can be utilized as a breast cancer diagnostic. The results of any test indicative of breast cancer known in the art may be combined with the methods described herein. Examples of such tests are mammography (e.g., a more frequent and/or earlier mammography regimen may be prescribed); breast biopsy and optionally a biopsy from another tissue; breast ultrasound and optionally an ultrasound analysis of another tissue; breast magnetic resonance imaging (MRI) and optionally an MRI analysis of another tissue; electrical impedance (T-scan) analysis of breast and optionally of another tissue; ductal lavage; nuclear medicine analysis (e.g., scintimammography); BRCA1 and/or BRCA2 sequence analysis results; and thermal imaging of the breast and optionally of another tissue. Testing may be performed on tissue other than breast to diagnose the occurrence of metastasis (e.g., testing of the lymph node).

Risk of breast cancer sometimes is expressed as a probability, such as an odds ratio, percentage, or risk factor. The risk is based upon the presence or absence of one or more polymorphic variants described herein, and also may be based in part upon phenotypic traits of the individual being tested. Methods for calculating predispositions based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations. Risk determinations for breast cancer are useful in a variety of applications. In one embodiment, breast cancer risk determinations are used by clinicians to direct appropriate detection, preventative and treatment procedures to subjects who most require these. In another embodiment, breast cancer risk determinations are used by health insurers for preparing actuarial tables and for calculating insurance premiums.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a nucleotide sequence set forth in SEQ ID NO: 1-7 without undue experimentation using knowledge readily available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2. Multiple extension oligonucleotides may be utilized in one reaction, which is referred to herein as “multiplexing.”

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site set forth in SEQ ID NO: 1-7 or below.

A kit also may be utilized for determining whether a polymorphic variant is present or absent in a nucleic acid sample. A kit often comprises one or more pairs of oligonucleotide primers useful for amplifying a fragment of a ICAM, MAPK10, KIAA0861, NUMA1, GALE, DPF3 or LOC145197 nucleotide sequence or a substantially identical sequence thereof, where the fragment includes a polymorphic site. The kit sometimes comprises a polymerizing agent, for example, a thermostable nucleic acid polymerase such as one disclosed in U.S. Pat. Nos. 4,889,818 or 6,077,664. Also, the kit often comprises an elongation oligonucleotide that hybridizes to a ICAM, MAPK10, KIAA0861, NUMA1, GALE, DPF3 or LOC145197 nucleotide sequence in a nucleic acid sample adjacent to the polymorphic site. Where the kit includes an elongation oligonucleotide, it also often comprises chain elongating nucleotides, such as DATP, dTTP, dGTP, dCTP, and dITP, including analogs of DATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

An individual identified as being at risk of breast cancer may be heterozygous or homozygous with respect to the allele associated with a higher risk of breast cancer. A subject homozygous for an allele associated with an increased risk of breast cancer is at a comparatively high risk of breast cancer, a subject heterozygous for an allele associated with an increased risk of breast cancer is at a comparatively intermediate risk of breast cancer, and a subject homozygous for an allele associated with a decreased risk of breast cancer is at a comparatively low risk of breast cancer. A genotype may be assessed for a complementary strand, such that the complementary nucleotide at a particular position is detected.

Also featured are methods for determining risk of breast cancer and/or identifying a subject at risk of breast cancer by contacting a polypeptide or protein encoded by a ICAM, MAPK10, KIAA0861, NUMA1, GALE, DPF3 or LOC145197 nucleotide sequence from a subject with an antibody that specifically binds to an epitope associated with increased risk of breast cancer in the polypeptide. In certain embodiments, the antibody specifically binds to an epitope that comprises a leucine at amino acid position 359 in SEQ ID NO: 23, a leucine at amino acid position 378 in SEQ ID NO: 23, an alanine at amino acid position 857 in SEQ ID NO: 23, an alanine at position 902 in SEQ ID NO: 23 a proline at amino acid position 352 in SEQ ID NO: 20 or an alanine at amino acid position 348 in SEQ ID NO: 20.

Applications of Prognostic and Diagnostic Results to Pharmacogenomic Methods

Pharmacogenomics is a discipline that involves tailoring a treatment for a subject according to the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects). As therapeutic approaches for breast cancer continue to evolve and improve, the goal of treatments for breast cancer related disorders is to intervene even before clinical signs (e.g., identification of lump in the breast) first manifest. Thus, genetic markers associated with susceptibility to breast cancer prove useful for early diagnosis, prevention and treatment of breast cancer.

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for detecting, preventing, alleviating and/or treating breast cancer. For example, a nucleic acid sample from an individual may be subjected to a genetic test described herein. Where one or more polymorphic variations associated with increased risk of breast cancer are identified in a subject, information for detecting, preventing or treating breast cancer and/or one or more breast cancer detection, prevention and/or treatment regimens then may be directed to and/or prescribed to that subject.

In certain embodiments, a detection, prevenative and/or treatment regimen is specifically prescribed and/or administered to individuals who will most benefit from it based upon their risk of developing breast cancer assessed by the methods described herein. Thus, provided are methods for identifying a subject at risk of breast cancer and then prescribing a detection, therapeutic or preventative regimen to individuals identified as being at risk of breast cancer. Thus, certain embodiments are directed to methods for treating breast cancer in a subject, reducing risk of breast cancer in a subject, or early detection of breast cancer in a subject, which comprise: detecting the presence or absence of a polymorphic variant associated with breast cancer in a nucleotide sequence in a nucleic acid sample from a subject, where the nucleotide sequence comprises a polynucleotide sequence selected from the group consisting of: (a) a nucleotide sequence set forth in SEQ ID NO: 1-7; (b) a nucleotide sequence which encodes a polypeptide having an amino acid sequence encoded by a nucleotide sequence in SEQ ID NO: 1-7; (c) a nucleotide sequence which encodes a polypeptide that is 90% or more identical to an amino acid sequence encoded by a nucleotide sequence in SEQ ID NO: 1-7 or a nucleotide sequence about 90% or more identical to the nucleotide sequence set forth in SEQ ID NO: 1-7; and (d) a fragment of a nucleotide sequence of (a), (b), or (c), sometimes comprising a polymorphic site associated with breast cancer; and prescribing or administering a breast cancer treatment regimen, preventative regimen and/or detection regimen to a subject from whom the sample originated where the presence of one or more polymorphic variations associated with breast cancer are detected in the nucleotide sequence. In these methods, genetic results may be utilized in combination with other test results to diagnose breast cancer as described above. Other test results include but are not limited to mammography results, imaging results, biopsy results and results from BRCA1 or BRAC2 test results, as described above.

Detection regimens include one or more mammography procedures, a regular mammography regimen (e.g., once a year, or once every six, four, three or two months); an early mammography regimen (e.g., mammography tests are performed beginning at age 25, 30, or 35); one or more biopsy procedures (e.g., a regular biopsy regimen beginning at age 40); breast biopsy and biopsy from other tissue; breast ultrasound and optionally ultrasound analysis of another tissue; breast magnetic resonance imaging (MRI) and optionally MRI analysis of another tissue; electrical impedance (T-scan) analysis of breast and optionally another tissue; ductal lavage; nuclear medicine analysis (e.g., scintimammography); BRCA1 and/or BRCA2 sequence analysis results; and/or thermal imaging of the breast and optionally another tissue.

Treatments sometimes are preventative (e.g., is prescribed or administered to reduce the probability that a breast cancer associated condition arises or progresses), sometimes are therapeutic, and sometimes delay, alleviate or halt the progression of breast cancer. Any known preventative or therapeutic treatment for alleviating or preventing the occurrence of breast cancer is prescribed and/or administered. For example, certain preventative treatments often are prescribed to subjects having a predisposition to breast cancer and where the subject is not diagnosed with breast cancer or is diagnosed as having symptoms indicative of early stage breast cancer (e.g., stage I). For subjects not diagnosed as having breast cancer, any preventative treatments known in the art can be prescribed and administered, which include selective hormone receptor modulators (e.g., selective estrogen receptor modulators (SERMs) such as tamoxifen, reloxifene, and toremifene); compositions that prevent production of hormones (e.g., aramotase inhibitors that prevent the production of estrogen in the adrenal gland, such as exemestane, letrozole, anastrozol, groserelin, and megestrol); other hormonal treatments (e.g., goserelin acetate and fulvestrant); biologic response modifiers such as antibodies (e.g., trastuzumab (herceptin/HER2)); surgery (e.g., lumpectomy and mastectomy); drugs that delay or halt metastasis (e.g., pamidronate disodium); and alternative/complementary medicine (e.g., acupuncture, acupressure, moxibustion, qi gong, reiki, ayurveda, vitamins, minerals, and herbs (e.g., astragalus root, burdock root, garlic, green tea, and licorice root)).

The use of breast cancer treatments are well known in the art, and include surgery, chemotherapy and/or radiation therapy. Any of the treatments may be used in combination to treat or prevent breast cancer (e.g., surgery followed by radiation therapy or chemotherapy). Examples of chemotherapeutics are taxanes (e.g., docetaxel or paclitaxel), and examples of chemotherapy combinations used to treat breast cancer include: cyclophosphamide (Cytoxan), methotrexate (Amethopterin, Mexate, Folex), and fluorouracil (Fluorouracil, 5-Fu, Adrucil), which is referred to as CMF; cyclophosphamide, doxorubicin (Adriamycin), and fluorouracil, which is referred to as CAF; and doxorubicin (Adriamycin) and cyclophosphamide, which is referred to as AC.

As breast cancer preventative and treatment information can be specifically targeted to subjects in need thereof (e.g., those at risk of developing breast cancer or those that have early signs of breast cancer), provided herein is a method for preventing or reducing the risk of developing breast cancer in a subject, which comprises: (a) detecting the presence or absence of a polymorphic variation associated with breast cancer at a polymorphic site in a nucleotide sequence in a nucleic acid sample from a subject; (b) identifying a subject with a predisposition to breast cancer, whereby the presence of the polymorphic variation is indicative of a predisposition to breast cancer in the subject; and (c) if such a predisposition is identified, providing the subject with information about methods or products to prevent or reduce breast cancer or to delay the onset of breast cancer. Also provided is a method of targeting information or advertising to a subpopulation of a human population based on the subpopulation being genetically predisposed to a disease or condition, which comprises: (a) detecting the presence or absence of a polymorphic variation associated with breast cancer at a polymorphic site in a nucleotide sequence in a nucleic acid sample from a subject; (b) identifying the subpopulation of subjects in which the polymorphic variation is associated with breast cancer; and (c) providing information only to the subpopulation of subjects about a particular product which may be obtained and consumed or applied by the subject to help prevent or delay onset of the disease or condition.

Pharmacogenomics methods also may be used to analyze and predict a response to a breast cancer treatment or a drug. For example, if pharmacogenomics analysis indicates a likelihood that an individual will respond positively to a breast cancer treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The methods described herein also are applicable to clinical drug trials. One or more polymorphic variants indicative of response to an agent for treating breast cancer or to side effects to an agent for treating breast cancer may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems. In certain embodiments, the agent for treating breast cancer described herein targets ICAM, MAPK10, KIAA0861, NUMA1, GALE, DPF3 or LOC145197 or a target in the ICAM, MAPK10, KIAA0861, NUMA1, GALE, DPF3 or LOC145197 pathway.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1-7; (ii) a polynucleotide sequence that is 90% or more identical to a nucleotide sequence set forth in SEQ ID NO: 1-7; (iii) a polynucleotide sequence that encodes a polypeptide having an amino acid sequence identical to or 90% or more identical to an amino acid sequence encoded by a nucleotide sequence set forth in SEQ ID NO: 1-7; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with breast cancer at a polymorphic site in a nucleotide sequence in a nucleic acid sample from a subject; (b) the diagnostic/prognostic testing provider identifies the subpopulation of subjects in which the polymorphic variation is associated with breast cancer; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Breast Cancer-Directed Molecules

Featured herein is a composition comprising a breast cancer cell and one or more molecules specifically directed and targeted to a nucleic acid comprising a ICAM, MAPK10, KIAA0861, NUMA1, GALE, DPF3 or LOC145197 nucleotide sequence or a ICAM, MAPK10, KIAA0861, NUMA1, GALE, DPF3 or LOC145197 polypeptide. Such directed molecules include, but are not limited to, a compound that binds to a ICAM, MAPK10, KIAA0861, NUMA1, GALE, DPF3 or LOC145197 nucleic acid or a ICAM, MAPK10, KIAA0861, NUMA1, GALE, DPF3 or LOC145197 polypeptide; a RNAi or siRNA molecule having a strand complementary to a ICAM, MAPK10, KIAA0861, NUMA1, GALE, DPF3 or LOC145197 nucleotide sequence; an antisense nucleic acid complementary to an RNA encoded by a ICAM, MAPK10, KIAA0861, NUMA1, GALE, DPF3 or LOC145197 DNA sequence; a ribozyme that hybridizes to a ICAM, MAPK10, KIAA0861, NUMA1, GALE, DPF3 or LOC145197 nucleotide sequence; a nucleic acid aptamer that specifically binds a ICAM, MAPK10, KIAA0861, NUMA1, GALE, DPF3 or LOC145197 polypeptide; and an antibody that specifically binds to a ICAM, MAPK10, KIAA0861, NUMA1, GALE, DPF3 or LOC145197 polypeptide or binds to a ICAM, MAPK10, KIAA0861, NUMA1, GALE, DPF3 or LOC145197 nucleic acid. In certain embodiments, the antibody specifically binds to an epitope that comprises a leucine at amino acid position 359 in SEQ ID NO: 23, a leucine at amino acid position 378 in SEQ ID NO: 23, an alanine at amino acid position 857 in SEQ ID NO: 23, an alanine at position 902 in SEQ ID NO: 23 a proline at amino acid position 352 in SEQ ID NO: 20 or an alanine at amino acid position 348 in SEQ ID NO: 20. In specific embodiments, the breast cancer directed molecule interacts with a ICAM, MAPK10, KIAA0861, NUMA1, GALE, DPF3 or LOC145197 nucleic acid or polypeptide variant associated with breast cancer. In other embodiments, the breast cancer directed molecule interacts with a polypeptide involved in the ICAM, MAPK10, KIAA0861, NUMA1, GALE, DPF3 or LOC145197 signal pathway, or a nucleic acid encoding such a polypeptide. Polypeptides involved in the ICAM, MAPK10, KIAA0861, NUMA1, GALE, DPF3 or LOC145197 signal pathway are discussed herein.

Compositions sometimes include an adjuvant known to stimulate an immune response, and in certain embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (IL)-12 and/or gamma-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethlene/polyoxypropylene block copolymer (Syntex Adjuvant Formulation); TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a breast cancer directed molecule (e.g., an HLA-binding subsequence within a polypeptide encoded by a nucleotide sequence in SEQ ID NO: 1). In such methods, a peptide having an amino acid subsequence of a polypeptide encoded by a nucleotide sequence in SEQ ID NO: 1-7 is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The breast cancer cell may be in a group of breast cancer cells and/or other types of cells cultured in vitro or in a tissue having breast cancer cells (e.g., a melanocytic lesion) maintained in vitro or present in an animal in vivo (e.g., a rat, mouse, ape or human). In certain embodiments, a composition comprises a component from a breast cancer cell or from a subject having a breast cancer cell instead of the breast cancer cell or in addition to the breast cancer cell, where the component sometimes is a nucleic acid molecule (e.g., genomic DNA), a protein mixture or isolated protein, for example. The aforementioned compositions have utility in diagnostic, prognostic and pharmacogenomic methods described previously and in breast cancer therapeutics described hereafter. Certain breast cancer molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37:1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a ICAM, MAPK10, KIAA0861, NUMA1, GALE, DPF3 or LOC145197 polypeptide and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire coding strand in SEQ ID NO: 1-17, or to a portion thereof or a substantially identical sequence thereof. In another embodiment, the antisense nucleic acid molecule is antisense to a “noncoding region” of the coding strand of a nucleotide sequence in SEQ ID NO: 1-17 (e.g., 5′ and 3′ untranslated regions).

An antisense nucleic acid can be designed such that it is complementary to the entire coding region of an mRNA encoded by a nucleotide sequence in SEQ ID NO: 1-7 (e.g., SEQ ID NO: 8-14), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target a nucleotide sequence in SEQ ID NO: 1-17, often a variant associated with breast cancer, or a substantially identical sequence thereof. Among the variants, minor alleles and major alleles can be targeted, and those associated with a higher risk of breast cancer are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a ICAM, MAPK10, KIAA0861, NUMA1, GALE, DPF3 or LOC145197 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al., U.S. Pat. No. 4,987,071; and Cech et al. U.S. Pat. No. 5,116,742). Also, target mRNA sequences can be used to select a catalytic RNA having a specific ribonuclease activity from a pool of RNA molecules (see e.g., Bartel & Szostak, Science 261: 1411-1418 (1993)).

Breast cancer directed molecules include in certain embodiments nucleic acids that can form triple helix structures with a ICAM, MAPK10, KIAA0861, NUMA1, GALE, DPF3 or LOC145197 nucleotide sequence or a substantially identical sequence thereof, especially one that includes a regulatory region that controls expression of a polypeptide. Gene expression can be inhibited by targeting nucleotide sequences complementary to the regulatory region of a ICAM, MAPK10, KIAA0861, NUMA1, GALE, DPF3 or LOC145197 nucleotide sequence or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Breast cancer directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February;2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. 2001 Aug. 14;98(17): 9742-7 and Elbashir et al. Methods 2002 February;26(2): 199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. Patent Application No. US2001000993183; Caplen et al. Proc Natl Acad Sci USA; and Abderrahmani et al. Mol Cell Biol 2001 Nov. 21(21): 7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al.,. Methods 26: 199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (SEQ ID NO: 4927)(N, any nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g., PCT Publication No. W089/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988) ). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a nucleotide sequence of SEQ ID NO: 1-17 or a substantially identical sequence thereof, two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. For example, antibodies that specifically bind to ICAM1 are disclosed in U.S. Pat. Nos. 5,475,091 and 5,773,293. An antibody may have effector function and can fix complement, and is sometimes coupled to a toxin or imaging agent.

A full-length polypeptide or antigenic peptide fragment encoded by a ICAM, MAPK10, KIAA0861, NUMA1, GALE, DPF3 or LOC145197 nucleotide sequence can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence of SEQ ID NO: 1-17, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

Antibody can be a single chain antibody. A single chain antibody (scFV) can be engineered (see, e.g., Colcher et al., Ann. NY Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat breast cancer, and as a therapeutic for administration to a subject for treating breast cancer.

An antibody can be made by immunizing with a purified antigen, or a fragment thereof e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by selecting antibodies that bind to native but not denatured polypeptide. Also featured are antibodies that specifically bind to a polypeptide variant associated with breast cancer.

Screening Assays

Featured herein are methods for identifying a candidate therapeutic for treating breast cancer. The methods comprise contacting a test molecule with a target molecule in a system. A “target molecule” as used herein refers to a nucleic acid of SEQ ID NO: 1-17, a substantially identical nucleic acid thereof, or a fragment thereof, and an encoded polypeptide of the foregoing. The method also comprises determining the presence or absence of an interaction between the test molecule and the target molecule, where the presence of an interaction between the test molecule and the nucleic acid or polypeptide identifies the test molecule as a candidate breast cancer therapeutic. The interaction between the test molecule and the target molecule may be quantified.

Test molecules and candidate therapeutics include, but are not limited to, compounds, antisense nucleic acids, siRNA molecules, ribozymes, polypeptides or proteins encoded by a ICAM, MAPK10, KIAA0861, NUMA1, GALE, DPF3 or LOC145197 nucleic acids, or a substantially identical sequence or fragment thereof, and immunotherapeutics (e.g., antibodies and HLA-presented polypeptide fragments). A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g., DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of breast cancer).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of an interaction between a test molecule and a target molecule. For example, titrametric, acidimetric, radiometric, NMR, monolayer, polarographic, spectrophotometric, fluorescent, and ESR assays probative of a target molecule interaction may be utilized.

In general, an interaction can be determined by labeling the test molecule and/or the ICAM, MAPK10, KIAA0861, NUMA1, GALE, DPF3 or LOC145197 molecule, where the label is covalently or non-covalently attached to the test molecule or ICAM, MAPK10, KIAA0861, NUMA1, GALE, DPF3 or LOC145197 molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and ICAM, MAPK10, KIAA0861, NUMA1, GALE, DPF3 or LOC145197 (McConnell, H. M. et al., Science 257: 1906-1912 (1992)).

In cell-based systems, cells typically include a ICAM, MAPK10, KIAA0861, NUMA1, GALE, DPF3 or LOC145197 nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a ICAM, MAPK10, KIAA0861, NUMA1, GALE, DPF3 or LOC145197 polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between two molecules also can be detected by monitoring fluorescence energy transfer (FET) (see, for example, Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

In another embodiment, determining the presence or absence of an interaction between a test molecule and a ICAM, MAPK10, KIAA0861, NUMA1, GALE, DPF3 or LOC145197 molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the ICAM, MAPK10, KIAA0861, NUMA1, GALE, DPF3 or LOC145197 molecule or test molecules are anchored to a solid phase. The ICAM, MAPK10, KIAA0861, NUMA1, GALE, DPF3 or LOC145197 molecule/test molecule complexes anchored to the solid phase can be detected at the end of the reaction. The target ICAM, MAPK10, KIAA0861, NUMA1, GALE, DPF3 or LOC145197 molecule is often anchored to a solid surface, and the test molecule, which is not anchored, can be labeled, either directly or indirectly, with detectable labels discussed herein.

It may be desirable to immobilize a ICAM, MAPK10, KIAA0861, NUMA1, GALE, DPF3 or LOC145197 molecule, an anti-ICAM, MAPK10, KIAA0861, NUMA1, GALE, DPF3 or LOC145197 antibody, or test molecules to facilitate separation of complexed from uncomplexed forms of ICAM, MAPK10, KIAA0861, NUMA1, GALE, DPF3 or LOC145197 molecules and test molecules, as well as to accommodate automation of the assay. Binding of a test molecule to a ICAM, MAPK10, KIAA0861, NUMA1, GALE, DPF3 or LOC145197 molecule can be accomplished in any vessel suitable for containing the reactants. Examples of such vessels include microtiter plates, test tubes, and micro-centrifuge tubes. In one embodiment, a fusion polypeptide can be provided which adds a domain that allows a ICAM, MAPK10, KIAA0861, NUMA1, GALE, DPF3 or LOC145197 molecule to be bound to a matrix. For example, glutathione-S-transferase/ICAM, MAPK10, KIAA0861, NUMA1, GALE, DPF3 or LOC145197 fusion polypeptides or glutathione-S-transferase/target fusion polypeptides can be adsorbed onto glutathione sepharose beads (Sigma Chemical, St. Louis, Mo.) or glutathione derivitized microtiter plates, which are then combined with the test compound or the test compound and either the non-adsorbed target polypeptide or ICAM, MAPK10, KIAA0861, NUMA1, GALE, DPF3 or LOC145197 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of ICAM, MAPK10, KIAA0861, NUMA1, GALE, DPF3 or LOC145197 binding or activity determined using standard techniques.

Other techniques for immobilizing a ICAM, MAPK10, KIAA0861, NUMA1, GALE, DPF3 or LOC145197 molecule on matrices include using biotin and streptavidin. For example, biotinylated ICAM, MAPK10, KIAA0861, NUMA1, GALE, DPF3 or LOC145197 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with ICAM, MAPK10, KIAA0861, NUMA1, GALE, DPF3 or LOC145197 polypeptide or test molecules but which do not interfere with binding of the ICAM, MAPK10, KIAA0861, NUMA1, GALE, DPF3 or LOC145197 polypeptide to its test molecule. Such antibodies can be derivitized to the wells of the plate, and unbound target or ICAM, MAPK10, KIAA0861, NUMA1, GALE, DPF3 or LOC145197 polypeptide trapped in the wells by antibody conjugation. Methods for detecting such complexes, in addition to those described above for the GST-immobilized complexes, include immunodetection of complexes using antibodies reactive with the ICAM, MAPK10, KIAA0861, NUMA1, GALE, DPF3 or LOC145197 polypeptide or target molecule, as well as enzyme-linked assays which rely on detecting an enzymatic activity associated with the ICAM, MAPK10, KIAA0861, NUMA1, GALE, DPF3 or LOC145197 polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August; 18(8): 284-7 (1993)); chromatography (gel filtration chromatography, ion-exchange chromatography); electrophoresis (see, e.g., Ausubel et al., eds. Current Protocols in Molecular Biology, J. Wiley: New York (1999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, J Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of ICAM, MAPK10, KIAA0861, NUMA1, GALE, DPF3 or LOC145197 expression are identified. For example, a cell or cell free mixture is contacted with a candidate compound and the expression of ICAM, MAPK10, KIAA0861, NUMA1, GALE, DPF3 or LOC145197 mRNA or polypeptide evaluated relative to the level of expression of ICAM, MAPK10, KIAA0861, NUMA1, GALE, DPF3 or LOC145197 mRNA or polypeptide in the absence of the candidate compound. When expression of ICAM, MAPK10, KIAA0861, NUMA1, GALE, DPF3 or LOC145197 mRNA or polypeptide is greater in the presence of the candidate compound than in its absence, the candidate compound is identified as a stimulator of ICAM, MAPK10, KIAA0861, NUMA1, GALE, DPF3 or LOC145197 mRNA or polypeptide expression. Alternatively, when expression of ICAM, MAPK10, KIAA0861, NUMA1, GALE, DPF3 or LOC145197 mRNA or polypeptide is less (statistically significantly less) in the presence of the candidate compound than in its absence, the candidate compound is identified as an inhibitor of ICAM, MAPK10, KIAA0861, NUMA1, GALE, DPF3 or LOC145197 mRNA or polypeptide expression. The level of ICAM, MAPK10, KIAA0861, NUMA1, GALE, DPF3 or LOC145197 mRNA or polypeptide expression can be determined by methods described herein for detecting ICAM, MAPK10, KIAA0861, NUMA1, GALE, DPF3 or LOC145197 mRNA or polypeptide.

In another embodiment, binding partners that interact with a ICAM, MAPK10, KIAA0861, NUMA1, GALE, DPF3 or LOC145197 molecule are detected. The ICAM, MAPK10, KIAA0861, NUMA1, GALE, DPF3 or LOC145197 molecules can interact with one or more cellular or extracellular macromolecules, such as polypeptides, in vivo, and these molecules that interact with ICAM, MAPK10, KIAA0861, NUMA1, GALE, DPF3 or LOC145197 molecules are referred to herein as “binding partners.” Molecules that disrupt such interactions can be useful in regulating the activity of the target gene product. Such molecules can include, but are not limited to molecules such as antibodies, peptides, and small molecules. Target genes/products for use in this embodiment often are the ICAM, MAPK10, KIAA0861, NUMA1, GALE, DPF3 or LOC145197 genes herein identified. In an alternative embodiment, provided is a method for determining the ability of the test compound to modulate the activity of a ICAM, MAPK10, KIAA0861, NUMA1, GALE, DPF3 or LOC145197 polypeptide through modulation of the activity of a downstream effector of a ICAM, MAPK10, KIAA0861, NUMA1, GALE, DPF3 or LOC145197 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases where it is desirable to identify compounds that disrupt interactions of mutant but not normal target gene products. Examples of ICAM1 inhibitors are Actimid and Bortezomib, and U.S. Pat. No. 6,436,403 describes a conjugate between ICAM1 and a virus for ICAM1 delivery. Examples of MAPK10 and JUNK modulators are beta arrestin (ARBB2), which is an agonist, CC-401 (Celgene), which is an inhibitor, and other modulators disclosed in WO0246184A1, WO02083667A2, WO02079197A1, WO0112621A1, WO04005283A1, WO0210137A2, WO0112609A1, WO03099221A2, WO02066450A2, WO03102151A2, US20020160397A1. Examples of MAPK10 screening methods are disclosed in U.S. Pat. No. 6,610,505.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of ICAM, MAPK10, KIAA0861, NUMA1, GALE, DPF3 or LOC145197 molecules can be identified in a two-hybrid assay or three-hybrid assay (see, e.g., U.S. Pat. No. 5,283,317; Zervos et al., Cell 72: 223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with ICAM, MAPK10, KIAA0861, NUMA1, GALE, DPF3 or LOC145197 (“ICAM, MAPK10, KIAA0861, NUMA1, GALE, DPF3 or LOC145197-binding polypeptides” or “ICAM, MAPK10, KIAA0861, NUMA1, GALE, DPF3 or LOC145197-bp”) and are involved in ICAM, MAPK10, KIAA0861, NUMA1, GALE, DPF3 or LOC145197 activity. Such ICAM, MAPK10, KIAA0861, NUMA1, GALE, DPF3 or LOC145197-bps can be activators or inhibitors of signals by the ICAM, MAPK10, KIAA0861, NUMA1, GALE, DPF3 or LOC145197 polypeptides or ICAM, MAPK10, KIAA0861, NUMA1, GALE, DPF3 or LOC145197 targets as, for example, downstream elements of a ICAM, MAPK10, KIAA0861, NUMA1, GALE, DPF3 or LOC145197-mediated signaling pathway.

A two-hybrid system is based on the modular nature of most transcription factors, which consist of separable DNA-binding and activation domains. Briefly, the assay utilizes two different DNA constructs. In one construct, the gene that codes for a ICAM, MAPK10, KIAA0861, NUMA1, GALE, DPF3 or LOC145197 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: ICAM, MAPK10, KIAA0861, NUMA1, GALE, DPF3 or LOC145197 polypeptide can be the fused to the activator domain.) If the “bait” and the “prey” polypeptides are able to interact, in vivo, forming a ICAM, MAPK10, KIAA0861, NUMA1, GALE, DPF3 or LOC14519 7-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the ICAM, MAPK10, KIAA0861, NUMA1, GALE, DPF3 or LOC145197 polypeptide.

Candidate therapeutics for treating breast cancer are identified from a group of test molecules that interact with a ICAM, MAPK10, KIAA0861, NUMA1, GALE, DPF3 or LOC145197 nucleic acid or polypeptide. Test molecules are normally ranked according to the degree with which they interact or modulate (e.g., agonize or antagonize) DNA replication and/or processing, RNA transcription and/or processing, polypeptide production and/or processing, and/or function of ICAM, MAPK10, KIAA0861, NUMA1, GALE, DPF3 or LOC145197 molecules, for example, and then top ranking modulators are selected. In a preferred embodiment, the candidate therapeutic (i.e., test molecule) acts as a ICAM, MAPK10, KIAA0861, NUMA1, GALE, DPF3 or LOC145197 antagonist. Also, pharmacogenomic information described herein can determine the rank of a modulator. Candidate therapeutics typically are formulated for administration to a subject.

Therapeutic Treatments

Formulations or pharmaceutical compositions typically include in combination with a pharmaceutically acceptable carrier, a compound, an antisense nucleic acid, a ribozyme, an antibody, a binding partner that interacts with a ICAM, MAPK10, KIAA0861, NUMA1, GALE, DPF3 or LOC145197 polypeptide, a ICAM, MAPK10, KIAA0861, NUMA1, GALE, DPF3 or LOC145197 nucleic acid, or a fragment thereof. The formulated molecule may be one that is identified by a screening method described above. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, sodium chloride sometimes are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules often lies within a range of circulating concentrations that include the ED₅₀ with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment, or sometimes can include a series of treatments.

With regard to polypeptide formulations, featured herein is a method for treating breast cancer in a subject, which comprises contacting one or more cells in the subject with a first ICAM, MAPK10, KIAA0861, NUMA1, GALE, DPF3 or LOC145197 polypeptide, where the subject comprises a second ICAM, MAPK10, KIAA0861, NUMA1, GALE, DPF3 or LOC145197 polypeptide having one or more polymorphic variations associated with cancer, and where the first polypeptide comprises fewer polymorphic variations associated with cancer than the second polypeptide. The first and second polypeptides are encoded by a nucleic acid which comprises a nucleotide sequence selected from the group consisting of the nucleotide sequence of SEQ ID NO: 1-17; a nucleotide sequence which encodes a polypeptide consisting of an amino acid sequence encoded by a nucleotide sequence of SEQ ID NO: 1-17; a nucleotide sequence which encodes a polypeptide that is 90% or more identical to an amino acid sequence encoded by a nucleotide sequence of SEQ ID NO: 1-17 and a nucleotide sequence 90% or more identical to a nucleotide sequence of SEQ ID NO: 1-17. The subject is often a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14: 193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

ICAM, MAPK10, KIAA0861, NUMA1, GALE, DPF3 or LOC145197 nucleic acid molecules can be inserted into vectors and used in gene therapy methods for treating breast cancer. Featured herein is a method for treating breast cancer in a subject, which comprises contacting one or more cells in the subject with a first ICAM, MAPK10, KIAA0861, NUMA1, GALE, DPF3 or LOC145197 nucleic acid, where genomic DNA in the subject comprises a second ICAM, MAPK10, KIAA0861, NUMA1, GALE, DPF3 or LOC145197 nucleic acid comprising one or more polymorphic variations associated with breast cancer, and where the first nucleic acid comprises fewer polymorphic variations associated with breast cancer. The first and second nucleic acids typically comprise a nucleotide sequence selected from the group consisting of the nucleotide sequence of SEQ ID NO: 1-7; a nucleotide sequence which encodes a polypeptide consisting of an amino acid sequence encoded by a nucleotide sequence in SEQ ID NO: 1-7; a nucleotide sequence that is 90% or more identical to the nucleotide sequence of SEQ ID NO: 1-7, and a nucleotide sequence which encodes a polypeptide that is 90% or more identical to an amino acid sequence encoded by a nucleotide sequence in SEQ ID NO: 1-7. The subject often is a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91: 3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for treating breast cancer. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the ICAM, MAPK10, KIAA0861, NUMA1, GALE, DPF3 or LOC145197 aberrance, such that a disease or disorder is prevented or, alternatively, delayed in its progression. Depending on the type of ICAM, MAPK10, KIAA0861, NUMA1, GALE, DPF3 or LOC145197 aberrance, for example, a ICAM, MAPK10, KIAA0861, NUMA1, GALE, DPF3 or LOC145197molecule, ICAM, MAPK10, KIAA0861, NUMA1, GALE, DPF3 or LOC145197 agonist, or ICAM, MAPK10, KIAA0861, NUMA1, GALE, DPF3 or LOC145197 antagonist agent can be used for treating the subject. The appropriate agent can be determined based on screening assays described herein.

As discussed, successful treatment of ICAM, MAPK10, KIAA0861, NUMA1, GALE, DPF3 or LOC145197 disorders can be brought about by techniques that serve to inhibit the expression or activity of target gene products. For example, compounds (e.g., an agent identified using an assays described above) that exhibit negative modulatory activity can be used to prevent and/or treat breast cancer. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)2 and FAb expression library fragments, scFV molecules, and epitope-binding fragments thereof).

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances where the target gene encodes an extracellular polypeptide, normal target gene polypeptide often is co-administered into the cell or tissue to maintain the requisite level of cellular or tissue target gene activity.

Another method by which nucleic acid molecules may be utilized in treating or preventing a disease characterized by ICAM, MAPK10, KIAA0861, NUMA1, GALE, DPF3 or LOC145197 expression is through the use of aptamer molecules specific for ICAM, MAPK10, KIAA0861, NUMA1, GALE, DPF3 or LOC145197 polypeptide. Aptamers are nucleic acid molecules having a tertiary structure which permits them to specifically bind to polypeptide ligands (see, e.g., Osborne, et al., Curr. Opin. Chem. Biol. 1(1): 5-9 (1997); and Patel, D. J., Curr. Opin. Chem. Biol. June;1(1): 32-46 (1997)). Since nucleic acid molecules may in many cases be more conveniently introduced into target cells than therapeutic polypeptide molecules may be, aptamers offer a method by which ICAM, MAPK10, KIAA0861, NUMA1, GALE, DPF3 or LOC145197 polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of ICAM, MAPK10, KIAA0861, NUMA1, GALE, DPF3 or LOC145197 disorders. For a description of antibodies, see the Antibody section above.

In circumstances where injection of an animal or a human subject with a ICAM, MAPK10, KIAA0861, NUMA1, GALE, DPF3 or LOC145197 polypeptide or epitope for stimulating antibody production is harmful to the subject, it is possible to generate an immune response against ICAM, MAPK10, KIAA0861, NUMA1, GALE, DPF3 or LOC145197 through the use of anti-idiotypic antibodies (see, for example, Herlyn, D., Ann. Med.; 31(1): 66-78 (1999); and Bhattacharya-Chatterjee & Foon, Cancer Treat. Res.; 94: 51-68 (1998)). If an anti-idiotypic antibody is introduced into a mammal or human subject, it should stimulate the production of anti-anti-idiotypic antibodies, which should be specific to the ICAM, MAPK10, KIAA0861, NUMA1, GALE, DPF3 or LOC145197 polypeptide. Vaccines directed to a disease characterized by ICAM, MAPK10, KIAA0861, NUMA1, GALE, DPF3 or LOC145197 expression may also be generated in this fashion.

In instances where the target antigen is intracellular and whole antibodies are used, internalizing antibodies may be utilized. Lipofectin or liposomes can be used to deliver the antibody or a fragment of the Fab region that binds to the target antigen into cells. Where fragments of the antibody are used, the smallest inhibitory fragment that binds to the target antigen often is utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-7893 (1993)).

ICAM, MAPK10, KIAA0861, NUMA1, GALE, DPF3 or LOC145197 molecules and compounds that inhibit target gene expression, synthesis and/or activity can be administered to a patient at therapeutically effective doses to prevent, treat or ameliorate ICAM, MAPK10, KIAA0861, NUMA1, GALE, DPF3 or LOC145197 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices often are utilized. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds often lies within a range of circulating concentrations that include the ED₅₀ with little or no toxicity. The dosage can vary within this range depending upon the dosage form employed and the route of administration utilized. For any compound used in a method described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. The compound which is able to modulate ICAM, MAPK10, KIAA0861, NUMA1, GALE, DPF3 or LOC145197 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates the expression or activity of ICAM, MAPK10, KIAA0861, NUMA1, GALE, DPF3 or LOC145197 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al., Analytical Chemistry 67: 2142-2144 (1995).

Provided herein are methods of modulating ICAM, MAPK10, KIAA0861, NUMA1, GALE, DPF3 or LOC145197 expression or activity for therapeutic purposes. Accordingly, in an exemplary embodiment, the modulatory method involves contacting a cell with a ICAM, MAPK10, KIAA0861, NUMA1, GALE, DPF3 or LOC145197 or agent that modulates one or more of the activities of ICAM, MAPK10, KIAA0861, NUMA1, GALE, DPF3 or LOC145197 polypeptide activity associated with the cell. An agent that modulates ICAM, MAPK10, KIAA0861, NUMA1, GALE, DPF3 or LOC145197 polypeptide activity can be an agent as described herein, such as a nucleic acid or a polypeptide, a naturally-occurring target molecule of a ICAM, MAPK10, KIAA0861, NUMA1, GALE, DPF3 or LOC145197 polypeptide (e.g., a ICAM, MAPK10, KIAA0861, NUMA1, GALE, DPF3 or LOC145197 substrate or receptor), a ICAM, MAPK10, KIAA0861, NUMA1, GALE, DPF3 or LOC145197 antibody, a ICAM, MAPK10, KIAA0861, NUMA1, GALE, DPF3 or LOC145197 agonist or antagonist, a peptidomimetic of a ICAM, MAPK10, KIAA0861, NUMA1, GALE, DPF3 or LOC145197 agonist or antagonist, or other small molecule.

In one embodiment, the agent stimulates one or more ICAM, MAPK10, KIAA0861, NUMA1, GALE, DPF3 or LOC145197 activities. Examples of such stimulatory agents include active ICAM, MAPK10, KIAA0861, NUMA1, GALE, DPF3 or LOC145197 polypeptide and a nucleic acid molecule encoding ICAM, MAPK10, KIAA0861, NUMA1, GALE, DPF3 or LOC145197. In another embodiment, the agent inhibits one or more ICAM, MAPK10, KIAA0861, NUMA1, GALE, DPF3 or LOC145197 activities. Examples of such inhibitory agents include antisense ICAM, MAPK10, KIAA0861, NUMA1, GALE, DPF3 or LOC145197 nucleic acid molecules, anti-ICAM, MAPK10, KIAA0861, NUMA1, GALE, DPF3 or LOC145197 antibodies, and ICAM, MAPK10, KIAA0861, NUMA1, GALE, DPF3 or LOC145197 inhibitors, and competitive inhibitors that target ICAM, MAPK10, KIAA0861, NUMA1, GALE, DPF3 or LOC145197. These modulatory methods can be performed in vitro (e.g., by culturing the cell with the agent) or, alternatively, in vivo (e.g., by administering the agent to a subject). As such, provided are methods of treating an individual afflicted with a disease or disorder characterized by aberrant or unwanted expression or activity of a ICAM, MAPK10, KIAA0861, NUMA1, GALE, DPF3 or LOC145197 polypeptide or nucleic acid molecule. In one embodiment, the method involves administering an agent (e.g., an agent identified by a screening assay described herein), or combination of agents that modulates (e.g., upregulates or downregulates) ICAM, MAPK10, KIAA0861, NUMA1, GALE, DPF3 or LOC145197 expression or activity. In another embodiment, the method involves administering a ICAM, MAPK10, KIAA0861, NUMA1, GALE, DPF3 or LOC145197 polypeptide or nucleic acid molecule as therapy to compensate for reduced, aberrant, or unwanted ICAM, MAPK10, KIAA0861, NUMA1, GALE, DPF3 or LOC145197 expression or activity.

Stimulation of ICAM, MAPK10, KIAA0861, NUMA1, GALE, DPF3 or LOC145197 activity is desirable in situations in which ICAM, MAPK10, KIAA0861, NUMA1, GALE, DPF3 or LOC145197 is abnormally downregulated and/or in which increased ICAM, MAPK10, KIAA0861, NUMA1, GALE, DPF3 or LOC145197 activity is likely to have a beneficial effect. For example, stimulation of ICAM, MAPK10, KIAA0861, NUMA1, GALE, DPF3 or LOC145197 activity is desirable in situations in which a ICAM, MAPK10, KIAA0861, NUMA1, GALE, DPF3 or LOC145197 is downregulated and/or in which increased ICAM, MAPK10, KIAA0861, NUMA1, GALE, DPF3 or LOC145197 activity is likely to have a beneficial effect. Likewise, inhibition of ICAM, MAPK10, KIAA0861, NUMA1, GALE, DPF3 or LOC145197 activity is desirable in situations in which ICAM, MAPK10, KIAA0861, NUMA1, GALE, DPF3 or LOC145197 is abnormally upregulated and/or in which decreased ICAM, MAPK10, KIAA0861, NUMA1, GALE, DPF3 or LOC145197 activity is likely to have a beneficial effect.

Methods of Treatment

In another aspect, provided are methods for identifying a risk of cancer in an individual as described herein and, if a genetic predisposition is identified, treating that individual to delay or reduce or prevent the development of cancer. Such a procedure can be used to treat breast cancer. Optionally, treating an individual for cancer may include inhibiting cellular proliferation, inhibiting metastasis, inhibiting invasion, or preventing tumor formation or growth as defined herein. Suitable treatments to prevent or reduce or delay breast cancer focus on inhibiting additional cellular proliferation, inhibiting metastasis, inhibiting invasion, and preventing further tumor formation or growth. Treatment usually includes surgery followed by radiation therapy. Surgery may be a lumpectomy or a mastectomy (e.g., total, simple or radical). Even if the doctor removes all of the cancer that can be seen at the time of surgery, the patient may be given radiation therapy, chemotherapy, or hormone therapy after surgery to try to kill any cancer cells that may be left. Radiation therapy is the use of x-rays or other types of radiation to kill cancer cells and shrink tumors. Radiation therapy may use external radiation (using a machine outside the body) or internal radiation. Chemotherapy is the use of drugs to kill cancer cells. Chemotherapy may be taken by mouth, or it may be put into the body by inserting a needle into a vein or muscle. Hormone therapy often focuses on estrogen and progesterone, which are hormones that affect the way some cancers grow. If tests show that the cancer cells have estrogen and progesterone receptors (molecules found in some cancer cells to which estrogen and progesterone will attach), hormone therapy is used to block the way these hormones help the cancer grow. Hormone therapy with tamoxifen is often given to patients with early stages of breast cancer and those with metastatic breast cancer. Other types of treatment being tested in clinical trials include sentinel lymph node biopsy followed by surgery and high-dose chemotherapy with bone marrow transplantation and peripheral blood stem cell transplantation. Any preventative/therapeutic treatment known in the art may be prescribed and/or administered, including, for example, surgery, chemotherapy and/or radiation treatment, and any of the treatments may be used in combination with one another to treat or prevent breast cancer (e.g., surgery followed by radiation therapy).

Also provided are methods of preventing or treating cancer comprising providing an individual in need of such treatment with a ICAM, MAPK10, KIAA0861, NUMA1, GALE, DPF3 or LOC145197 inhibitor that reduces or inhibits the overexpression of mutant ICAM, MAPK10, KIAA0861, NUMA1, GALE, DPF3 or LOC145197 (e.g., a ICAM, MAPK10, KIAA0861, NUMA1, GALE, DPF3 or LOC145197 polynucleotide with an allele that is associated with cancer). Included herein are methods of reducing or blocking the expression of ICAM, MAPK10, KIAA0861, NUMA1, GALE, DPF3 or LOC145197 comprising providing or administering to individuals in need of reducing or blocking the expression of ICAM, MAPK10, KIAA0861, NUMA1, GALE, DPF3 or LOC145197 a pharmaceutical or physiologically acceptable composition comprising a molecule capable of inhibiting expression of ICAM, MAPK10, KIAA0861, NUMA1, GALE, DPF3 or LOC145197, e.g., a siRNA molecule. Also included herein are methods of reducing or blocking the expression of secondary regulatory genes regulated by ICAM, MAPK10, KIAA0861, NUMA1, GALE, DPF3 or LOC145197 that play a role in oncogenesis which comprises introducing competitive inhibitors that target the effect of ICAM, MAPK10, KIAA0861, NUMA1, GALE, DPF3 or LOC145197 on these regulatory genes or that block the binding of positive factors necessary for the expression of these regulatory genes.

The examples set forth below are intended to illustrate but not limit the invention.

### Examples

In the following studies a group of subjects were selected according to specific parameters relating to breast cancer. Nucleic acid samples obtained from individuals in the study group were subjected to genetic analysis, which identified associations between breast cancer and certain polymorphic variants in ICAM region, MAPK10, KIAA0861, NUMA1/FLJ20625/LOC220074 region, and HT014/LOC148902/LYPLA2/GALE region loci (herein referred to as “target genes”, “target nucleotides”, “target polypeptides” or simply “targets”). In addition, methods are described for combining information from multiple SNPs from the target genes found to be independently associated with breast cancer status in a case-control study. The resulting model permits a powerful, more informative quantitation of the combined value of the SNPs for predicting breast cancer susceptibility.

### Example 1

### Samples and Pooling Strategies

### Sample Selection

Blood samples were collected from individuals diagnosed with breast cancer, which were referred to as case samples. Also, blood samples were collected from individuals not diagnosed with breast cancer as gender and age-matched controls. All of the samples were of German/German descent. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1× TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtitre plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtitre plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of two groups based on disease status. The two groups were female case groups and female control groups. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy). Samples that met these criteria were added to appropriate pools based on gender and disease status.

The selection process yielded the pools set forth in Table 1, which were used in the studies that follow:

TABLE 1

| | Female CASE | Female CONTROL |
|---|---|---|
| Pool size | 272 | 276 |
| (Number) | | |
| Pool Criteria | case | control |
| (ex: case/control) | | |
| Mean Age | 59.6 | 55.4 |
| (ex: years) | | |

### Example 2

### Association of Polymorphic Variants with Breast Cancer

A whole-genome screen was performed to identify particular SNPs associated with occurrence of breast cancer. As described in Example 1, two sets of samples were utilized, which included samples from female individuals having breast cancer (breast cancer cases) and samples from female individuals not having cancer (female controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with breast cancer when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p-value was calculated to determine whether the case and control groups had statistically significantly differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensemble genes; and they are located in Genomatix promoter predictions. SNPs in the set also were selected on the basis of even spacing across the genome, as depicted in Table 2.

A case-control study design using a whole genome association strategy involving approximately 28,000 single nucleotide polymorphisms (SNPs) was employed. Approximately 25,000 SNPs were evenly spaced in gene-based regions of the human genome with a median inter-marker distance of about 40,000 base pairs. Additionally, approximately 3,000 SNPs causing amino acid substitutions in genes described in the literature as candidates for various diseases were used. The case-control study samples were of female German origin (German paternal and maternal descent) 548 individuals were equally distributed in two groups (female controls and female cases). The whole genome association approach was first conducted on 2 DNA pools representing the 2 groups. Significant markers were confirmed by individual genotyping.

TABLE 2

| General Statistics | | Spacing Statistics | | |
|---|---|---|---|---|
| Total # of SNPs | 28,532 | Median | 34,424 | bp |
| # of Exonic SNPs | 8,497 (30%) | Minimum* | 1,000 | bp |
| # SNPs with refSNP ID | 26,625 (93%) | Maximum* | 3,000,000 | bp |
| Gene Coverage | 23,874 | Mean | 122,412 | bp |
| Chromosome Coverage | All | Std | 354, | bp |
| | | Deviation | | |

*Excludes outliers

### Allelotyping and Genotyping Results

The genetic studies summarized above and described in more detail below identified allelic variants associated with breast cancer. The allelic variants identified from the SNP panel described in Table 2 are summarized below in Table 3.

TABLE 3

| | | | Position | | | | | | |
|---|---|---|---|---|---|---|---|---|---|
| SNP | | Chromosome | in SEQ ID | Contig | Contig | Sequence | | Sequence | Allelic |
| Reference | Chromosome | Position | NO: 1-7 | Identification | Position | Identification | Locus | Position | Variability |
| 11549918 | 19 | 10263938 | 44247 | NT_011295 | 1665740 | NM_003259 | ICAM region | Exon | C/T |
| | | | | | | | | (I301V) | |
| 1541998 | 4 | 87408539 | 36424 | NT_016354 | 11444849 | NM_002753 | MAPK10 | intron | C/T |
| 2001449 | 3 | 184049849 | 48563 | NT_005612 | 89424094 | NM_015078 | KIAA0861 | intron | G/C |
| 673478 | 11 | 71525412 | 49002 | NT_033927 | 1998133 | NM_006185 | NUMA1 | upstream | T/C |
| | | | | | | NM_017907 | FLJ20625 | downstream | |
| | | | | | | NM_145309 | LOC220074 | intron | |
| 4237 | 1 | 23583604 | 87877 | | | NM_000403 | GALE | downstream | A/G |
| | | | | NT_004610 | 4917379 | NM_020362 | HT014 | 3′utr | |
| | | | | | | NM_007260 | LYPLA2 | upstream | |
| 1990440 | 14 | 71267195 | 40095 | NT_026437 | 53197195 | NM_012074 | DPF3 | intron | G/C |
| 1054745 | 14 | 99900494 | | NT_026437 | 81830494 | XM_096734 | LOC145197 | upstream | G/A |

Table 3 includes information pertaining to the incident polymorphic variant associated with breast cancer identified herein. Public information pertaining to the polymorphism and the genomic sequence that includes the polymorphism are indicated. The genomic sequences identified in Table 3 may be accessed on the World Wide Web at the URL “ncbi.nih.gov/entrez/query.fcgi,” for example, by using the publicly available SNP reference number (e.g., rs1541998). The chromosome position refers to the position of the SNP within NCBI's Genome Build 34, which may be accessed on the World Wide Web at the URL “ncbi.nlm.nih.gov/mapview/map_search.cgi?chr=hum_chr.inf&query=.” The “Contig Position” provided in Table 3 corresponds to a nucleotide position set forth in the contig sequence, and designates the polymorphic site corresponding to the SNP reference number. The sequence containing the polymorphisms also may be referenced by the “Sequence Identification” set forth in Table 3. The “Sequence Identification” corresponds to cDNA sequence that encodes associated target polypeptides (e.g., NUMA1) of the invention. The position of the SNP within the cDNA sequence is provided in the “Sequence Position” column of Table 3. Also, the allelic variation at the polymorphic site and the allelic variant identified as associated with breast cancer is specified in Table 3. All nucleotide sequences referenced and accessed by the parameters set forth in Table 3 are incorporated herein by reference. The positions for these SNPs are indicated in the tables below and in SEQ ID NO: 1-7.

Assay for Verifying, Allelotyping, and Genotyping SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer was used to genotype the polymorphism. Table 4 shows PCR primers (SEQ ID NOS 28-41, respectively, in order of appearance) and Table 5 shows extension primers (SEQ ID NOS 42-48, respectively, in order of appearance) used for analyzing polymorphisms. The initial PCR amplification reaction was performed in a 5 μl total volume containing 1× PCR buffer with 1.5 mM MgCl₂ (Qiagen), 200 μM each of DATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 4
PCR Primers

| Reference | Forward | Reverse |
|---|---|---|
| SNP ID | PCR primer | PCR primer |
| 11549918 | GACAGCCACAGCTAGCGCAGA | TGTTTTCGCCCCCCAGGG |
| | | TGAC |
| 1541998 | CTGATTATTCTGATGGTAATG | GCCCATGTTAACATTTT |
| | | CTTC |
| 2001449 | ATGTCAAGTGCACCCACATG | AGGAAGAAACTGACGG |
| | | AAGG |
| 673478 | TAATACAAAGGTGGCAGCAG | TTGACAAGGATAAGGA |
| | | CAAG |
| 4237 | GCACATGGCCACATTAACTGG | TGGCTGTGGAAATTGGGT |
| | | CTTG |
| 1990440 | CCAGGGTGTGTTCTAATACG | AAGTCACTAACCCCAC |
| | | ACAC |
| 1054745 | GACTTTTAGGTCTGAGTTGG | CTTCCTCTAGCAGTGTA |
| | | TTTC |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. In Table 5 (SEQ ID NOS 42-48, respectively, in order of appearance), ddNTPs are shown and the fourth nucleotide not shown is the dNTP.

TABLE 5
Extend Primers

| Reference | Extend | Term |
|---|---|---|
| SNP ID | Probe | Mix |
| 11549918 | CCCAGGGTGACGTTGCAGA | ACG |
| 1541998 | ATTATTCTGATGGTAATGATCCAG | ACG |
| 2001449 | CACATGCCTGCTCGCCCCC | ACT |
| 673478 | AAGGGGAGGTCGACTGGG | ACT |
| 4237 | GGCATCTGGCAGTCATGG | ACT |
| 1990440 | CGTCAGCAAATGTGTACCGA | ACT |
| 1054745 | TTGGTCCATTAGGGAATTAGA | ACG |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of DATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP® (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

Variations identified in the target genes are provided in their respective genomic sequences (see FIGS. 1-7) Minor allelic frequencies for these polymorphisms was verified as being 10% or greater by determining the allelic frequencies using the extension assay described above in a group of samples isolated from 92 individuals originating from the state of Utah in the United States, Venezuela and France (Coriell cell repositories).

Genotyping results are shown for female pools in Table 6A and 6B. Table 6A shows the original genotyping results and Table 6B shows the genotyped results re-analyzed to remove duplicate individuals from the cases and controls (i.e., individuals who were erroneously included more than once as either cases or controls). Therefore, Table 6B represents a more accurate measure of the allele frequencies for this particular SNP. In the subsequent tables, “AF” refers to allelic frequency; and “F case” and “F control” refer to female case and female control groups, respectively.

TABLE 6A

| | | | | | Breast |
|---|---|---|---|---|---|
| Reference | | | | Odds | Cancer |
| SNP ID | AF F case | AF F control | p-value | Ratio | Assoc. Allele |
| 11549918 | C = 0.651 | C = 0.564 | 0.0038 | 0.69 | C |
| | T = 0.349 | T = 0.436 | | | |
| 1541998 | T = 0.780 | T = 0.839 | 0.0153 | 0.69 | C |
| | C = 0.220 | C = 0.161 | | | |
| 2001449 | G = 0.703 | G = 0.780 | 0.0040 | 1.49 | C |
| | C = 0.297 | C = 0.220 | | | |
| 673478 | T = 0.919 | T = 0.953 | 0.0238 | 1.74 | C |
| | C = 0.081 | C = 0.047 | | | |
| 4237 | A = 0.590 | A = 0.530 | 0.0431 | 0.78 | A |
| | G = 0.410 | G = 0.470 | | | |
| 1990440 | C = 0.876 | C = 0.926 | 0.0027 | 0.65 | G |
| | G = 0.124 | G = 0.074 | | | |
| 1054745 | A = 0.871 | A = 0.805 | 0.0039 | | A |
| | G = 0.129 | G = 0.195 | | | |

TABLE 6B

| | | | | | Breast |
|---|---|---|---|---|---|
| Reference | | | | Odds | Cancer |
| SNP ID | AF F case | AF F control | p-value | Ratio | Assoc. Allele |
| 11549918 | C = 0.658 | C = 0.556 | 0.0012 | 0.65 | C |
| | T = 0.342 | T = 0.444 | | | |
| 1541998 | T = 0.771 | T = 0.839 | 0.0070 | 0.65 | C |
| | C = 0.229 | C = 0.161 | | | |
| 2001449 | G = 0.693 | G = 0.782 | 0.0012 | 1.59 | C |
| | C = 0.307 | C = 0.218 | | | |
| 673478 | T = 0.916 | T = 0.953 | 0.0171 | 1.85 | C |
| | C = 0.084 | C = 0.047 | | | |
| 4237 | A = 0.584 | A = 0.527 | 0.0704 | 0.79 | A |
| | G = 0.416 | G = 0.473 | | | |
| 1990440 | C = 0.879 | C = 0.915 | 0.0692 | 0.67 | G |
| | G = 0.121 | G = 0.085 | | | |
| 1054745 | A = 0.866 | A = 0.801 | 0.0061 | 1.60 | A |
| | G = 0.134 | G = 0.199 | | | |

The single marker alleles set forth in Table 3 were considered validated, since the genotyping data for the females, males or both pools were significantly associated with breast cancer, and because the genotyping results agreed with the original allelotyping results. Particularly significant associations with breast cancer are indicated by a calculated p-value of less than 0.05 for genotype results, which are set forth in bold text.

Odds ratio results are shown in Tables 6A and 6B. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:

RR=IA/Ia

IA is the incidence of disease in the A carriers and Ia is the incidence of disease in the non-carriers.

RR>1 indicates the A allele increases disease susceptibility.

RR<1 indicates the a allele increases disease susceptibility.

For example, RR=1.5 indicates that carriers of the A allele have 1.5 times the risk of disease than non-carriers, i.e., 50% more likely to get the disease.

Case-control studies do not allow the direct estimation of IA and Ia, therefore relative risk cannot be directly estimated. However, the odds ratio (OR) can be calculated using the following equation:

OR=(nDAnda)/(ndAnDa)=pDA(1−pdA)/pdA(1−pDA), or

OR=((case f)/(1−case f))/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of breast cancer. Possessing an allele associated with a relatively high odds ratio translates to having a higher risk of developing or having breast cancer.

### Example 3

### Samples and Pooling Strategies for the Replication Samples

The SNPs of Table 3 were genotyped again in a collection of replication samples to further validate its association with breast cancer. Like the original study population described in Examples 1 and 2, the replication samples consisted of females diagnosed with breast cancer (cases) and females without cancer (controls). The case and control samples were selected and genotyped as described below.

### Pooling Strategies

Samples were placed into one of two groups based on disease status. The two groups were female case groups and female control groups. A select set of samples from each group were utilized to generate pools, and one pool was created for each group. Each individual sample in a pool was represented by an equal amount of genomic DNA. For example, where 25 ng of genomic DNA was utilized in each PCR reaction and there were 190 individuals in each pool (i.e., 190 cases and 190 controls), each individual would provide 125 pg of genomic DNA. Inclusion or exclusion of samples for a pool was based upon the following criteria: the sample was derived from a female individual characterized as Caucasian from Australia; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Samples in the pools also were age-matched. Samples that met these criteria were added to appropriate pools based on gender and disease status.

The selection process yielded the “Griffith” samples set forth in Table 7A and the “Kiechle” samples set forth in Table 7B, which were used in the studies that follow:

TABLE 7A

| | Female CASE | Female CONTROL |
|---|---|---|
| Pool size | 190 | 190 |
| (Number) | | |
| Pool Criteria | case | control |
| (ex: case/control) | | |
| Mean Age | 64.5 | ** |
| (ex: years) | | |

**Each case was matched by a control within 5 years of age of the case.

TABLE 7B

| | Female CASE | Female CONTROL |
|---|---|---|
| Pool size | 195 | 153 |
| (Number) | | |
| Pool Criteria | case | control |
| (ex: case/control) | | |

The replication genotyping results are shown in Table 8A for the Griffith samples and in Table 8B for the Kiechle samples. The odds ratio was calculated as described in Example 2.

TABLE 8A

| Reference | AF | AF | | |
|---|---|---|---|---|
| SNP ID | F case | F control | p-value | Odds Ratio |
| 11549918 | C = 0.650 | C = 0.584 | 0.0624 | 0.75 |
| | T = 0.350 | T = 0.416 | | |
| 1541998 | T = 0.820 | T = 0.864 | 0.1010 | 0.72 |
| | C = 0.180 | C = 0.136 | | |
| 2001449 | G = 0.685 | G = 0.777 | 0.005 | 1.59 |
| | C = 0.315 | C = 0.223 | | |
| 673478 | T = 0.927 | T = 0.957 | 0.077 | 1.76 |
| | C = 0.073 | C = 0.043 | | |
| 4237 | A = 0.632 | A = 0.577 | 0.1260 | 1.26 |
| | G = 0.368 | G = 0.423 | | |
| 1990440 | C = 0.822 | C = 0.860 | 0.172 | 0.756 |
| | G = 0.178 | G = 0.140 | | |
| 1054745 | A = 0.894 | A = 0.921 | 0.224 | 0.726 |
| | G = 0.106 | G = 0.079 | | |

TABLE 8B

| Reference | AF | AF | | |
|---|---|---|---|---|
| SNP ID | F case | F control | p-value | Odds Ratio |
| 11549918 | C = 0.641 | C = 0.570 | 0.066 | 0.74 |
| | T = 0.359 | T = 0.430 | | |
| 1541998 | T = 0.811 | T = 0.791 | 0.521 | 1.14 |
| | C = 0.189 | C = 0.209 | | |
| 2001449 | G = 0.754 | G = 0.716 | 0.267 | 0.82 |
| | C = 0.246 | C = 0.284 | | |
| 673478 | T = 0.942 | T = 0.955 | 0.474 | 1.30 |
| | C = 0.058 | C = 0.045 | | |
| 4237 | A = 0.618 | A = 0.587 | 0.429 | 0.88 |
| | G = 0.382 | G = 0.413 | | |
| 1990440 | C = 0.889 | C = 0.924 | 0.124 | 0.66 |
| | G = 0.111 | G = 0.076 | | |
| 1054745 | A = 0.877 | A = 0.819 | 0.0396 | 1.57 |
| | G = 0.123 | G = 0.181 | | |

The absence of a statistically significant association in the replication cohort should not be interpreted as minimizing the value of the original finding. There are many reasons why a biologically derived association identified in a sample from one population would not replicate in a sample from another population. The most important reason is differences in population history. Due to bottlenecks and founder effects, there may be common disease predisposing alleles present in one population that are relatively rare in another, leading to a lack of association in the candidate region. Also, because common diseases such as breast cancer are the result of susceptibilities in many genes and many environmental risk factors, differences in population-specific genetic and environmental backgrounds could mask the effects of a biologically relevant allele. For these and other reasons, statistically strong results in the original, discovery sample that did not replicate in the replication sample may be further evaluated in additional replication cohorts and experimental systems.

### Example 4

### ICAM Region Proximal SNPs

It has been discovered that a polymorphic variation (rs11549918) in a region that encodes ICAM1, ICAM2 and ICAM5 is associated with the occurrence of breast cancer (see Examples 1 and 2). Subsequently, SNPs proximal to the incident SNP (rs11549918) were identified and allelotyped in breast cancer sample sets and control sample sets as described in Examples 1 and 2. Approximately 195 allelic variants located within the ICAM region were identified and allelotyped. The polymorphic variants are set forth in Table 9. The chromosome position provided in column four of Table 9 is based on Genome “Build 34” of NCBI's GenBank.

TABLE 9

| | Position in | | | Allele | Genome | Deduced |
|---|---|---|---|---|---|---|
| dbSNP rs# | SEQ ID NO: 1 | Chromosome | Chromosome Position | Variants | Letter | Iupac |
| 2884487 | 230 | 19 | 10219830 | t/c | c | Y |
| 2358580 | 4282 | 19 | 10223882 | a/g | g | R |
| 2304236 | 11844 | 19 | 10231444 | a/c | a | M |
| 1059840 | 11890 | 19 | 10231490 | a/t | t | W |
| 1059843 | 11905 | 19 | 10231505 | c/t | c | Y |
| 11115 | 11942 | 19 | 10231542 | t/c | c | Y |
| 1059849 | 12054 | 19 | 10231654 | g/a | a | R |
| 1059855 | 12070 | 19 | 10231670 | t/c | c | Y |
| 5030386 | 21497 | 19 | 10241097 | c/g | c | S |
| 5030339 | 21537 | 19 | 10241137 | a/g | g | R |
| 5030387 | 21682 | 19 | 10241282 | t/g | t | K |
| 5030388 | 22073 | 19 | 10241673 | a/g | g | R |
| 1799766 | 22393 | 19 | 10241993 | c/— | c | N |
| 5030389 | 22394 | 19 | 10241994 | a/— | a | N |
| 5490 | 23227 | 19 | 10242827 | c/a | a | M |
| 11575070 | 23614 | 19 | 10243214 | c/g | g | S |
| 5030340 | 23681 | 19 | 10243281 | c/t | c | Y |
| 5030390 | 23937 | 19 | 10243537 | a/g | g | R |
| 5030391 | 24037 | 19 | 10243637 | a/g | g | R |
| 3093035 | 24373 | 19 | 10243973 | a/g | g | R |
| 11667983 | 24589 | 19 | 10244189 | c/t | c | Y |
| 5030341 | 24675-24676 | 19 | 10244275\hat{ }10244276 | ct/— | ct | NN |
| 5030342 | 24680 | 19 | 10244280 | t/g | g | K |
| 5030343 | 24754 | 19 | 10244354 | t/c | c | Y |
| 5030344 | 25053 | 19 | 10244653 | g/a | g | R |
| 5030347 | 26004 | 19 | 10245604 | a/c | c | M |
| 5030348 | 26023 | 19 | 10245623 | a/g | a | R |
| 5030349 | 26046 | 19 | 10245646 | t/c | c | Y |
| 5030350 | 26100 | 19 | 10245700 | a/t | a | W |
| 5030351 | 26817 | 19 | 10246417 | t/c | c | Y |
| 5491 | 26940 | 19 | 10246540 | t/a | a | W |
| 5030352 | 27143 | 19 | 10246743 | g/c | c | S |
| 5030353 | 27162 | 19 | 10246762 | a/g | g | R |
| 10420063 | 28328 | 19 | 10247928 | g/a | a | R |
| 11879117 | 28799 | 19 | 10248399 | g/c | g | S |
| 5030354 | 29166 | 19 | 10248766 | a/c | a | M |
| 5030355 | 29331 | 19 | 10248931 | a/c | a | M |
| 281428 | 29724 | 19 | 10249324 | c/t | c | Y |
| 5030358 | 29773 | 19 | 10249373 | g/a | a | R |
| 5030359 | 29862 | 19 | 10249462 | a/g | g | R |
| 5030392 | 29877 | 19 | 10249477 | t/c | c | Y |
| 5030393 | 30194 | 19 | 10249794 | t/g | g | K |
| 5030360 | 30258 | 19 | 10249858 | t/g | g | K |
| 5030394 | 30549 | 19 | 10250149 | a/g | g | R |
| 281429 | 30648 | 19 | 10250248 | t/c | c | Y |
| 5030361 | 30909 | 19 | 10250509 | t/c | c | Y |
| 5030362 | 30962 | 19 | 10250562 | a/c | c | M |
| 281430 | 31337 | 19 | 10250937 | g/a | a | R |
| 281431 | 31345 | 19 | 10250945 | t/c | t | Y |
| 5030395 | 31534 | 19 | 10251134 | c/t | c | Y |
| 5827095 | 31942 | 19 | 10251542 | a/— | a | N |
| 281432 | 32058 | 19 | 10251658 | g/c | c | S |
| 5030364 | 32109 | 19 | 10251709 | a/t | t | W |
| 5030365 | 32401 | 19 | 10252001 | a/g | a | R |
| 5030368 | 32845 | 19 | 10252445 | g/a | a | R |
| 5030369 | 32864 | 19 | 10252464 | t/c | c | Y |
| 3073809 | 32965-32966 | 19 | 10252565\hat{ }10252566 | tt/— | tt | NN |
| 2358581 | 33011 | 19 | 10252611 | g/t | t | K |
| 7258215 | 33170 | 19 | 10252770 | c/t | t | Y |
| 5030371 | 33516 | 19 | 10253116 | a/g | g | R |
| 5030372 | 33608 | 19 | 10253208 | t/c | t | Y |
| 281433 | 33623 | 19 | 10253223 | a/c | a | M |
| 5030374 | 33672 | 19 | 10253272 | c/a | a | M |
| 5030375 | 33724 | 19 | 10253324 | a/— | a | N |
| 5030397 | 33787 | 19 | 10253387 | t/g | g | K |
| 281434 | 34020 | 19 | 10253620 | g/a | a | R |
| 12462944 | 34038 | 19 | 10253638 | c/g | g | S |
| 5030398 | 34190 | 19 | 10253790 | c/g | c | S |
| 5030378 | 34706 | 19 | 10254306 | c/g | c | S |
| 12459133 | 34778 | 19 | 10254378 | t/g | g | K |
| 5030399 | 35553 | 19 | 10255153 | a/g | g | R |
| 5492 | 35690 | 19 | 10255290 | g/c | g | S |
| 1800019 | 36173 | 19 | 10255773 | a/c | c | M |
| 1799969 | 36192 | 19 | 10255792 | g/a | g | R |
| 5493 | 36194 | 19 | 10255794 | t/g | g | K |
| 5030381 | 36308 | 19 | 10255908 | c/t | c | Y |
| 5494 | 36317 | 19 | 10255917 | c/t | c | Y |
| 3093033 | 36431 | 19 | 10256031 | g/a | g | R |
| 5495 | 36496 | 19 | 10256096 | g/a | g | R |
| 1801714 | 36608 | 19 | 10256208 | c/t | c | Y |
| 2071441 | 36708 | 19 | 10256308 | c/t | c | Y |
| 5496 | 36847 | 19 | 10256447 | a/g | g | R |
| 5497 | 36868 | 19 | 10256468 | a/g | g | R |
| 5030382 | 37083 | 19 | 10256683 | g/a | a | R |
| 5030400 | 37196 | 19 | 10256796 | c/t | c | Y |
| 2071440 | 37373 | 19 | 10256973 | g/a | g | R |
| 5499 | 37465 | 19 | 10257065 | c/t | c | Y |
| 3093032 | 37736 | 19 | 10257336 | t/c | c | Y |
| 1057981 | 37959 | 19 | 10257559 | g/a | a | R |
| 5500 | 38031 | 19 | 10257631 | a/g | a | R |
| 5501 | 38091 | 19 | 10257691 | t/c | t | Y |
| 5030383 | 38243 | 19 | 10257843 | t/c | c | Y |
| 281436 | 38531 | 19 | 10258131 | a/g | g | R |
| 923366 | 38623 | 19 | 10258223 | t/c | c | Y |
| 281437 | 38638 | 19 | 10258238 | c/t | c | Y |
| 3093030 | 38803 | 19 | 10258403 | t/c | c | Y |
| 5030384 | 39489 | 19 | 10259089 | a/g | g | R |
| 5030385 | 40008 | 19 | 10259608 | g/c | c | S |
| 3810159 | 40711 | 19 | 10260311 | t/c | g | R |
| 281438 | 40775 | 19 | 10260375 | t/g | t | K |
| 3093029 | 40951 | 19 | 10260551 | c/g | c | S |
| 2735442 | 41218 | 19 | 10260818 | g/c | g | S |
| 2569693 | 41304 | 19 | 10260904 | c/t | c | Y |
| 281439 | 41510 | 19 | 10261110 | g/c | g | S |
| 281440 | 41704 | 19 | 10261304 | g/a | g | R |
| 2569694 | 42020 | 19 | 10261620 | c/t | c | Y |
| 11575073 | 42210 | 19 | 10261810 | c/t | c | Y |
| 2569695 | 42302 | 19 | 10261902 | c/g | c | S |
| 2075741 | 42498 | 19 | 10262098 | g/c | g | S |
| 11575074 | 42520 | 19 | 10262120 | a/g | g | R |
| 2569696 | 42607 | 19 | 10262207 | a/g | g | R |
| 2735439 | 42674 | 19 | 10262274 | t/g | t | K |
| 2569697 | 42929 | 19 | 10262529 | g/c | g | S |
| 2075742 | 43007 | 19 | 10262607 | g/c | g | S |
| 2569698 | 43040 | 19 | 10262640 | c/g | c | S |
| 11669397 | 43246 | 19 | 10262846 | c/t | c | Y |
| 901886 | 43531 | 19 | 10263131 | t/c | t | Y |
| 885742 | 44023 | 19 | 10263623 | g/a | a | R |
| 2569699 | 44024 | 19 | 10263624 | t/g | t | K |
| 11549918 | 44338 | 19 | 10263938 | c/t | g | R |
| 2569700 | 44499 | 19 | 10264099 | t/g | g | K |
| 2228615 | 44768 | 19 | 10264368 | a/g | g | R |
| ICAM-AA | 45003 | 19 | 10264603 | c/t | c | Y |
| 2569701 | 45341 | 19 | 10264941 | g/a | g | R |
| 2569702 | 45347 | 19 | 10264947 | t/c | t | Y |
| 2735440 | 45577 | 19 | 10265177 | g/a | a | R |
| 2569703 | 45627 | 19 | 10265227 | c/g | c | S |
| 10418913 | 45794 | 19 | 10265394 | a/g | g | R |
| 1056536 | 46244 | 19 | 10265844 | c/t | g | R |
| 2569704 | 46469 | 19 | 10266069 | t/c | c | Y |
| 11673661 | 46999 | 19 | 10266599 | g/c | g | S |
| 10402760 | 47265 | 19 | 10266865 | t/c | c | Y |
| 0 | 47504 | 19 | 10267104 | a/g | g | R |
| 2569706 | 47547 | 19 | 10267147 | g/a | g | R |
| 2569707 | 47637 | 19 | 10267237 | c/g | g | S |
| 2436545 | 47691 | 19 | 10267291 | c/a | a | M |
| 2436546 | 47692 | 19 | 10267292 | a/c | a | M |
| 2916060 | 47788 | 19 | 10267388 | a/c | t | K |
| 2916059 | 47796 | 19 | 10267396 | t/c | a | R |
| 2916058 | 47800 | 19 | 10267400 | t/a | a | W |
| 2569708 | 47812 | 19 | 10267412 | a/g | g | R |
| 735747 | 47969 | 19 | 10267569 | t/c | t | Y |
| 885743 | 48035 | 19 | 10267635 | a/t | t | W |
| 710845 | 48569 | 19 | 10268169 | c/g | g | S |
| 2569709 | 48823 | 19 | 10268423 | c/g | c | S |
| 2569710 | 48838 | 19 | 10268438 | a/g | a | R |
| 2569711 | 48894 | 19 | 10268494 | c/g | c | S |
| 2569712 | 48934 | 19 | 10268534 | a/t | a | W |
| 12610026 | 49249 | 19 | 10268849 | a/g | g | R |
| 4804129 | 49509 | 19 | 10269109 | c/a | a | M |
| 12150978 | 49718 | 19 | 10269318 | a/g | a | R |
| 439843 | 49963 | 19 | 10269563 | c/a | c | M |
| 892188 | 51193 | 19 | 10270793 | t/c | c | Y |
| 2291473 | 57181 | 19 | 10276781 | t/c | t | Y |
| 281416 | 60184 | 19 | 10279784 | a/g | a | R |
| 281417 | 60530 | 19 | 10280130 | t/c | c | Y |
| 882589 | 61354 | 19 | 10280954 | t/c | g | R |
| 1048941 | 62616 | 19 | 10282216 | t/g | c | M |
| 281418 | 62785 | 19 | 10282385 | g/c | g | S |
| 430092 | 66351 | 19 | 10285951 | c/t | t | Y |
| 368835 | 67386 | 19 | 10286986 | a/g | a | R |
| 2358583 | 67395 | 19 | 10286995 | t/g | g | K |
| 378395 | 67822 | 19 | 10287422 | c/a | a | M |
| 395782 | 68028 | 19 | 10287628 | t/c | t | Y |
| 1045384 | 68646 | 19 | 10288246 | t/g | g | K |
| 281427 | 70520 | 19 | 10290120 | c/t | t | Y |
| 3745264 | 70966 | 19 | 10290566 | t/g | c | M |
| 281426 | 72451 | 19 | 10292051 | g/a | g | R |
| 281425 | 73199 | 19 | 10292799 | g/t | g | K |
| 281424 | 74319 | 19 | 10293919 | c/t | t | Y |
| 281423 | 76893 | 19 | 10296493 | c/t | t | Y |
| 281422 | 77755 | 19 | 10297355 | t/c | t | Y |
| 281420 | 79354 | 19 | 10298954 | a/g | a | R |
| 3745263 | 80901 | 19 | 10300501 | a/g | c | Y |
| 3745262 | 80940 | 19 | 10300540 | t/c | a | R |
| 3745261 | 81111 | 19 | 10300711 | t/c | a | R |
| 3181049 | 82517 | 19 | 10302117 | t/c | g | R |
| 281412 | 82874 | 19 | 10302474 | t/c | t | Y |
| 3181048 | 84978 | 19 | 10304578 | a/g | t | Y |
| 2230399 | 86003 | 19 | 10305603 | c/g | c | S |
| 2278442 | 86226 | 19 | 10305826 | g/a | g | R |
| 3181047 | 87206 | 19 | 10306806 | a/g | c | Y |
| 3181046 | 87535 | 19 | 10307135 | t/c | a | R |
| 2304237 | 87968 | 19 | 10307568 | t/c | t | Y |
| 281413 | 88134 | 19 | 10307734 | g/a | g | R |
| 1058154 | 88297 | 19 | 10307897 | a/c | c | M |
| 3176769 | 88434 | 19 | 10308034 | t/c | a | R |
| 2304238 | 90602 | 19 | 10310202 | g/a | a | R |
| 2304239 | 90750 | 19 | 10310350 | t/c | c | Y |
| 2304240 | 90792 | 19 | 10310392 | g/a | a | R |
| 3176768 | 91065 | 19 | 10310665 | a/g | c | Y |
| 3176767 | 91151 | 19 | 10310751 | c/a | t | K |
| 3176766 | 91178 | 19 | 10310778 | c/t | g | R |
| 281414 | 91685 | 19 | 10311285 | g/a | g | R |
| 281415 | 92393 | 19 | 10311993 | t/g | g | K |

### Assay for Verifying and Allelotyping SNPs

The methods used to verify and allelotype the proximal SNPs of Table 9 are the same methods described in Examples 1 and 2 herein. The PCR primers and extend primers used in these assays are provided in Table 10 (SEQ ID NOS 49-438, respectively, in order of appearance) and Table 11 (SEQ ID NOS 439-633, respectively, in order of appearance), respectively.

TABLE 10

| | | |
|---|---|---|
| dbSNP rs# | Forward PCR primer | Reverse PCR primer |

| | | |
|---|---|---|
| 2884487 | ACGTTGGATGTGTGGCAAATGATGGAACAG | ACGTTGGATGCCAGAAGTTTGAGATCTGCC |
| 2358580 | ACGTTGGATGTCGCGTGCGTGACGTCATCC | ACGTTGGATGCGGGAGGTCAAGGCCACTG |
| 2304236 | ACGTTGGATGTCACATCAACAGTGGTACGG | ACGTTGGATGAGCCTGCCCTACAGCGACTT |
| 1059840 | ACGTTGGATGTCGGCCTGGCTCAGAAGAGG | ACGTTGGATGACCCCTACCCCACGCTACCCA |
| 1059843 | ACGTTGGATGCCACCCCGTACCACTGTTGA | ACGTTGGATGTGAGGCTCCCAAGTCGGCCA |
| 11115 | ACGTTGGATGAGGTGACACCTTCCTCGAAG | ACGTTGGATGTGTGAAGCACCTCTTCTGAG |
| 1059849 | ACGTTGGATGGGAATGGATGCAGAAGCCCG | ACGTTGGATGAAGCTGAGGCCACAGGGAG |
| 1059855 | ACGTTGGATGAAAAACAAAAAGAGGGAATG | ACGTTGGATGGGCCATCGCCATTGGGAAG |
| 5030386 | ACGTTGGATGTTGAACTCCTGGGCTCAAGT | ACGTTGGATGAGACAAAAATCCACCTGGGC |
| 5030339 | ACGTTGGATGTCAAGTGATCCTCCCATCTC | ACGTTGGATGGGACACAGGAATGAACAGAG |
| 5030387 | ACGTTGGATGAAGAACGGCACCATTGTGGC | ACGTTGGATGTTCCACTGAGCTCTTGCCCC |
| 5030388 | ACGTTGGATGTCGGAGATCTCCATGGCGA | ACGTTGGATGACCACGTCGAGGCTGGGTAC |
| 1799766 | ACGTTGGATGGTTTCCCCGCGGAGCCTGG | ACGTTGGATGTCCCTACGCGCCGAGCCCC |
| 5030389 | ACGTTGGATGAAAAGGAAGGAAGCTGCGTG | ACGTTGGATGAAGCTAGCTGCCTCAGTTTC |
| 5490 | ACGTTGGATGCCTCTGCTACTCAGAGTTGC | ACGTTGGATGACTCACCTGGGAACAGAGC |
| 11575070 | ACGTTGGATGTAGCCTTACCTCCTGCCTCA | ACGTTGGATGACGCAATCTAGCCAAGCAAG |
| 5030340 | ACGTTGGATGATGAGAGGTGTGAACCACCG | ACGTTGGATGTGGCCGCTATAACGTTTCCC |
| 5030390 | ACGTTGGATGTTATCCGTGGCCCAAAGCTG | ACGTTGGATGAAAGACCCTCTGACCCCTTC |
| 5030391 | ACGTTGGATGTGTAAGTTTGGGGAACCAGG | ACGTTGGATGCTGGATCTCAGGCTTTGTTG |
| 3093035 | ACGTTGGATGGGAGACATAGCGAGATTCTG | ACGTTGGATGTAGAAAGCAGTGCGATCTGG |
| 11667983 | ACGTTGGATGATCTTTGAGTGAGGCAGTGC | ACGTTGGATGAGGGGCTTATTTGGATCAGG |
| 5030341 | ACGTTGGATGGAATCTGAGGGACCTGATCC | ACGTTGGATGTTGCAGTGAGCCGAGATCAC |
| 5030342 | ACGTTGGATGTTTTTGAGACGGAGTCTCAC | ACGTTGGATGAGGAGAATCGTTTGAACCTG |
| 5030343 | ACGTTGGATGTGTAGTCCCAGCTACTCAGG | ACGTTGGATGTGATCTCGGCTCACTGCAAC |
| 5030344 | ACGTTGGATGAAGAAGTCAGGTGGAGACAG | ACGTTGGATGAAATTGAGGCCCAAAGAGGG |
| 5030347 | ACGTTGGATGAATCTCAGCTCACTGCAACC | ACGTTGGATGGCCTGTAATCCCAGATACTC |
| 5030348 | ACGTTGGATGGGAGAATCACTTGAACCCGG | ACGTTGGATGAGTCTCGCTTTGTCACTCAG |
| 5030349 | ACGTTGGATGTCTCGCTTTGTCACTCAGAC | ACGTTGGATGAATCACTTGAACCCGGGAGG |
| 5030350 | ACGTTGGATGCACTGAAGGCCATGCTAAAG | ACGTTGGATGACTCCAGTCTGAGTGACAAA |
| 5030351 | ACGTTGGATGTGCCAGGTCCTAGAAGAAAC | ACGTTGGATGCGCACATTCCCTTGATGAAC |
| 5491 | ACGTTGGATGTGACATGCAGCACCTCCTGT | ACGTTGGATGGGAGCAACTCCTTTTTAGGC |
| 5030352 | ACGTTGGATGTACGAGCAAGTGGCAAAGATT | ACGTTGGATGTGAGTAACTGAGCCCGGAGG |
| 5030353 | ACGTTGGATGTACTGTGAGTAACTGAGCCC | ACGTTGGATGAGCAAGTGGCAAAGATTCCC |
| 10420063 | ACGTTGGATGATCTCCTGACCTTGTGATCC | ACGTTGGATGGCCCTGGGATGGCATTTTTC |
| 11879117 | ACGTTGGATGTGGTCAGGCTGGTCTCGAACT | ACGTTGGATGAAATGTCATGGGCTGGGCAC |
| 5030354 | ACGTTGGATGCTCTCTTCTCATAGCCAGCT | ACGTTGGATGATTAGGGAAGGGCAGTTTCG |
| 5030355 | ACGTTGGATGGGCCGGAGAATCACTTAAAC | ACGTTGGATGAGAGTTTCACTCTTGTTGCC |
| 281428 | ACGTTGGATGAGTAGCTGGAATTACAGGCG | ACGTTGGATGGCCAACATGATGAAATCCCG |
| 5030358 | ACGTTGGATGCACAAGGTCAGGAGATCAAG | ACGTTGGATGCCACGCCTGGCTAATTTTTG |
| 5030359 | ACGTTGGATGCCAAAGTGCTGGGATTACAG | ACGTTGGATGAAGTCCCCAAAGTAGGAAGG |
| 5030392 | ACGTTGGATGTTCGAAGTCCCCAAAGTAGG | ACGTTGGATGAAAGTGCTGGGATTACAGGC |
| 5030393 | ACGTTGGATGTAGTCCCAGCTACTCAGGAG | ACGTTGGATGTGTCATCACGGCTCACTACA |
| 5030360 | ACGTTGGATGTGTAGTGAGCCGTGATGACA | ACGTTGGATGTCCCAAAGCTACTTGGCCAG |
| 5030394 | ACGTTGGATGTGAGCAGATGCTGACCTTCC | ACGTTGGATGAAGACCTTCAAAGAGGTTTC |
| 281429 | ACGTTGGATGCGACAGAGCAAGACTGTGTG | ACGTTGGATGCTTTTCTGCATCTCTGCCTG |
| 5030361 | ACGTTGGATGTATGCTCTCAGCTATCAGGC | ACGTTGGATGACTCTAGACCTCTGGTGATC |
| 5030362 | ACGTTGGATGCCACAGTCATAGAGGGAGAA | ACGTTGGATGCCTGATAGCTGAGAGCATAG |
| 281430 | ACGTTGGATGATTACAGGTGTGAGCCACTG | ACGTTGGATGGCAGGGAGAAATCTTGATGG |
| 281431 | ACGTTGGATGACTGGGATTACAGGTGTGAG | ACGTTGGATGGGAGAAATCTTGATGGAGGC |
| 5030395 | ACGTTGGATGAACTACTCTGGAGGCTCAAG | ACGTTGGATGCAGAGTGAGACTCCATCTCA |
| 5827095 | ACGTTGGATGCCCTTCCTCCCATCAATCAA | ACGTTGGATGATTGCACCACTGCATTCCAG |
| 281432 | ACGTTGGATGTCAGCTATCTAATCCCTGGC | ACGTTGGATGAGCTGGGACTTTCCTTCTTG |
| 5030364 | ACGTTGGATGTTCTTGAATACGGTCACTGC | ACGTTGGATGCTGAAGGCTGGATTTACTGG |
| 5030365 | ACGTTGGATGAGCTGGAGGTCAGAGTTTTC | ACGTTGGATGCCTGAGACCCTGTCTCTAAA |
| 5030368 | ACGTTGGATGTCATCTGAGGTTGGGAGTTC | ACGTTGGATGCGACACCACACAAGGCTAAT |
| 5030369 | ACGTTGGATGAGGTGGATCATCTGAGGTTG | ACGTTGGATGGACAGGGTTTCTCCATGTTG |
| 3073809 | ACGTTGGATGACCGTGCCCGGCCTTTTTTT | ACGTTGGATGATCGAACCACTGCACTCCTG |
| 2358581 | ACGTTGGATGTAAGGCAGGAGGATGGAGTG | ACGTTGGATGGACAGAGTCTCACTCTGTCG |
| 7258215 | ACGTTGGATGGTTTCACCGTGTTAGCCAGG | ACGTTGGATGACATGGTGGCTCACGCCTGTA |
| 5030371 | ACGTTGGATGACTGGGATTACAGATGTGAG | ACGTTGGATGGTGCTTAAAAGGGATGAATC |
| 5030372 | ACGTTGGATGGTACATAAGCAGGAGGTTTC | ACGTTGGATGTCTGTACAAACAACCATGAC |
| 281433 | ACGTTGGATGACAGAGTCGCTGTACATAAG | ACGTTGGATGGTACAAACAACCATGACTAC |
| 5030374 | ACGTTGGATGTGTACAGCGACTCTGTCTAC | ACGTTGGATGCTCTGAACCTCAGTTTCCTG |
| 5030375 | ACGTTGGATGTCTCCTACCTTTAATAGCCC | ACGTTGGATGTTCTTTGCTGTGGGTAAGTG |
| 5030397 | ACGTTGGATGCTGGGTATCAAATGCAGGAC | ACGTTGGATGAGAGTGAGAGTCTCTCTCAG |
| 281434 | ACGTTGGATGTAATCCCAACACTTTGGGGG | ACGTTGGATGAACACCACGCCTGGCTAATT |
| 12462944 | ACGTTGGATGTTTCACTATATTGGCCAGGC | ACGTTGGATGTGTAATCCCAACACTTTGGG |
| 5030398 | ACGTTGGATGCCACACGCAGCCTTTTTGTT | ACGTTGGATGGAACTGTGATCATGCCACTG |
| 5030378 | ACGTTGGATGAGGCTTGTCTTGAACTCCTG | ACGTTGGATGTGATGGCTCAGGCTGTAATC |
| 12459133 | ACGTTGGATGAAAGTGCTAGGATTACAGCC | ACGTTGGATGTAAATGAGCCTGGCCTAGAC |
| 5030399 | ACGTTGGATGAGGTCCACTTCACCAGACAC | ACGTTGGATGTAAGGTTCTTGCCCACTGGC |
| 5492 | ACGTTGGATGACCGTGGTCGTGACCTCAG | ACGTTGGATGAACCTCACCGTGGTGCTGCT |
| 1800019 | ACGTTGGATGACAGCCCGTCCAGGGAACA | ACGTTGGATGTCCTAGAGGTGGACACGCA |
| 1799969 | ACGTTGGATGTCAACCTCTGGTCCCCCAGTG | ACGTTGGATGAGGGGACCGTGGTCTGTTC |
| 5493 | ACGTTGGATGTCCTAGAGGTGGACACGCAG | ACGTTGGATGTGGACCTGGGCCTCCGAGACT |
| 5030381 | ACGTTGGATGTATGGCAACGACTCCTTCTC | ACGTTGGATGTATTACTGCACACGTCAGCC |
| 5494 | ACGTTGGATGTAAGGCCTCAGTCAGTGTGA | ACGTTGGATGAGTATTACTGCACACGTCAG |
| 3093033 | ACGTTGGATGAAAGCCTGGAATAGGCACAC | ACGTTGGATGTGCAGACAGTGACCATCTAC |
| 5495 | ACGTTGGATGTCACACTTCACTGTCACCTC | ACGTTGGATGTGTGCCTATTCCAGGCTTTC |
| 1801714 | ACGTTGGATGCTAGAGCCAAGGTGACGCTG | ACGTTGGATGTGGCCTTCAGCAGGAGCTGG |
| 2071441 | ACGTTGGATGAGCTTCTCCTGCTCTGCAAC | ACGTTGGATGTCACACAGGACACGAAGCTC |
| 5496 | ACGTTGGATGAAGGTCTTGCCTCCAAGTCC | ACGTTGGATGACAATCCCTCTCGTCCAGTC |
| 5497 | ACGTTGGATGCTCCATGTCATCTCATCGTG | ACGTTGGATGAATTTTCTGGCCACGTCCAG |
| 5030382 | ACGTTGGATGCTCACAGAGCACATTCACGG | ACGTTGGATGAGATCTTGAGGGCACCTACC |
| 5030400 | ACGTTGGATGGGACCTAATGCAATCCTCAC | ACGTTGGATGGCTACCACAGTGATGATGAC |
| 2071440 | ACGTTGGATGACTGCCACCAATATGGGAAG | ACGTTGGATGAAACCGAACACACAAGCCAC |
| 5499 | ACGTTGGATGGGAAGACATATGCCATGCAG | ACGTTGGATGATCTGACTGAGGACAATGCC |
| 3093032 | ACGTTGGATGGGCCACTTCTTCTGTAAGTC | ACGTTGGATGCATGAGGACATACAACTGGG |
| 1057981 | ACGTTGGATGGTACAACTGTACCTGGTGAC | ACGTTGGATGAATGAACATAGGTCTCTGGC |
| 5500 | ACGTTGGATGTTGTACAGGTTGTACACTGC | ACGTTGGATGAGGTTGGCCAATGAGAAGTC |
| 5501 | ACGTTGGATGCTGTGAACCATTACCAGTCC | ACGTTGGATGACTTCTCATTGGCCAACCTG |
| 5030383 | ACGTTGGATGATATTCCCTGGGCACTCATG | ACGTTGGATGTCCCCACCCACATACATTTC |
| 281436 | ACGTTGGATGCATGGTTCACTGCAGTCTTG | ACGTTGGATGTGTGGTGTTGTGAGCCTATG |
| 923366 | ACGTTGGATGAGGAAGTCTGGGCAATGTTG | ACGTTGGATGATAGGCTCACAACACCACAC |
| 281437 | ACGTTGGATGATAGGCTCACAACACCACAC | ACGTTGGATGAACACAAAGGAAGTCTGGGC |
| 3093030 | ACGTTGGATGAGAGACCCAGAAGGTCATAG | ACGTTGGATGCCTCCCCCAAGAAAACATTG |
| 5030384 | ACGTTGGATGTGCGCAGGAAAAACACGCTG | ACGTTGGATGACTGTCAACTACCCTTCCCC |
| 5030385 | ACGTTGGATGATTAGGAACGGAACACAGGG | ACGTTGGATGAGAGAGTTATGACCCCGAGA |
| 3810159 | ACGTTGGATGTTGCGATCACTTTCTCCAAG | ACGTTGGATGCTGAGTGAGGAAACCTCAGG |
| 281438 | ACGTTGGATGACCTGAGGTTTCCTCACTCAG | ACGTTGGATGAGAGGTTTCTGTGACACCCG |
| 3093029 | ACGTTGGATGGGCAGCTCTGATTGGATGTT | ACGTTGGATGCTCCACAGTTGTTTGGCCTC |
| 2735442 | ACGTTGGATGATGCTTTTGACTCCGCTTCC | ACGTTGGATGATGTTCTGGTAGTCACCCAG |
| 2569693 | ACGTTGGATGCTTGTTCTCGCGTGGATGTC | ACGTTGGATGTACTCAGCGTGTGTGAGCTC |
| 281439 | ACGTTGGATGGCGGAGCCATACCTCTAAGC | ACGTTGGATGTCGCTGGCACTTTCGTCCC |
| 281440 | ACGTTGGATGCTGGCTGAGATGCCATGATA | ACGTTGGATGATGGTGGGAGGAGCTAAATG |
| 2569694 | ACGTTGGATGCGCCCCCTCCTTGGAAACC | ACGTTGGATGGCGCGGCGGGTGGGGTGTG |
| 11575073 | ACGTTGGATGGCTCTTCGGCCTCTCAGGT | ACGTTGGATGAGGCCGTTTCTCAGGTCCAG |
| 2569695 | ACGTTGGATGAGTCCCTGGACCTGAGAAAC | ACGTTGGATGAGAAGACTCAGGCTCGAGGT |
| 2075741 | ACGTTGGATGAAGATGCCAGTCCGTGGACC | ACGTTGGATGAGTGTCTCTCCTGTCCGCTC |
| 11575074 | ACGTTGGATGTACCTCCTTACGCTGTGCTG | ACGTTGGATGAGTTGGAGAGATGGAGTCTT |
| 2569696 | ACGTTGGATGAACTACCCTGACTCAGAGGC | ACGTTGGATGCTGGGCTCATGGAAGGAACC |
| 2735439 | ACGTTGGATGTTCCTTCCATGAGCCCAGCC | ACGTTGGATGGGTCAAGGATCAGGAAGGAC |
| 2569697 | ACGTTGGATGTGTCTCTGGGACGTGGTAGC | ACGTTGGATGTTTCCTCTCCTCTACGCCCT |
| 2075742 | ACGTTGGATGAGCCAGGGAAAGAACCCAGG | ACGTTGGATGTTTAGACTCTGGGAGTGCGC |
| 2569698 | ACGTTGGATGTTTAAACCGGAGGCCTGGGC | ACGTTGGATGTCAAGGGTTCTCAGAGGGCG |
| 11669397 | ACGTTGGATGGGCTGAATTGCAGCACCAAC | ACGTTGGATGAACCAACGCAAACCCCTCTG |
| 901886 | ACGTTGGATGATCAGCTCTACGCGATCTGG | ACGTTGGATGTTCAGGCCCCACCTTCTGTTC |
| 885742 | ACGTTGGATGCCTAAGCACTTGACATGCAC | ACGTTGGATGATGCTGACCCCGACTTCAAC |
| 2569699 | ACGTTGGATGAATGCTGACCCCGACTTCAA | ACGTTGGATGGCACTTGACATGCACTATCT |
| 11549918 | ACGTTGGATGTTTTCGCCCCCCAGGGTGAC | ACGTTGGATGACAGCCACAGCTAGCGCAGA |
| 2569700 | ACGTTGGATGGGGACCTCAGTACCGGAACA | ACGTTGGATGCTCCCCAGCGATTACGTCTC |
| 2228615 | ACGTTGGATGGGGCAGATGGTGACAGTAAC | ACGTTGGATGTGGAACTCCCTCCAGTGTGA |
| ICAM-AA | ACGTTGGATGACCTTCTGGTGACTTCCAAG | ACGTTGGATGAGACGTAAGAAATGGCTCCG |
| 2569701 | ACGTTGGATGCCAGACCTTGAACCAGATAG | ACGTTGGATGTCAAGGACGTAACGCTAACG |
| 2569702 | ACGTTGGATGACCCTCCAGACCTTGAACCA | ACGTTGGATGACGTAACGCTAACGGTGGAG |
| 2735440 | ACGTTGGATGTAGTCGAGCCATGCACTCTG | ACGTTGGATGGGCAGTCCAGAGTGTTTAAG |
| 2569703 | ACGTTGGATGTCAGGAAGCTCCCAGACAGA | ACGTTGGATGATAACCCTTGGACGCCGATC |
| 10418913 | ACGTTGGATGCCAAAGCCAACAATACACTC | ACGTTGGATGCAGCCAAGTAATGCGTTCTG |
| 1056536 | ACGTTGGATGATTCTGGGAGCTCTGGGACT | ACGTTGGATGAGCCAAGCGTGAAGTGCGTG |
| 2569704 | ACGTTGGATGGATTCATCCATCTCCGGTGG | ACGTTGGATGTCCAGGCACTCCCTGACATC |
| 11673661 | ACGTTGGATGAAGCGATTCTCCTGCCTCAG | ACGTTGGATGTGGCGAAAACCCGTCTCTAC |
| 10402760 | ACGTTGGATGAAGAGGATCCTGCCTCCTAT | ACGTTGGATGGAAGGGCGAGGAATTTTAGC |
| | ACGTTGGATGTGCGCCCAGGAGGAAACTTC | ACGTTGGATGCAGCTGATCTGGGCTGGAG |
| 2569706 | ACGTTGGATGCTGTTGTTGCTCGACAGGCC | ACGTTGGATGTGGCCTCCAGCCCAGATCA |
| 2569707 | ACGTTGGATGTGAGCGTGGCAGGCGCCATG | ACGTTGGATGGCGTGGCGCCCGTGCGCGT |
| 2436545 | ACGTTGGATGGGCGCATCACGGTGCGCGT | ACGTTGGATGAAGACCCAGGTCACGCCCCC |
| 2436546 | ACGTTGGATGTCTCAGATACCTCGCCCCGC | ACGTTGGATGCGCATCACGGTGCGCGTGG |
| 2916060 | ACGTTGGATGGGCGAGGTATCTGAGAGGG | ACGTTGGATGTACTCTGTCCCACTTCCGTC |
| 2916059 | ACGTTGGATGGGGCGAGGTATCTGAGAGG | ACGTTGGATGCTTTGACTTCTACTCTGTCC |
| 2916058 | ACGTTGGATGTCAAAGTGCGATCAACAGCC | ACGTTGGATGCTCTCGGTCCCGGTAGACT |
| 2569708 | ACGTTGGATGCGGTAGACTAGACGGAAGTG | ACGTTGGATGAAAGTGCGATCAACAGCCCC |
| 735747 | ACGTTGGATGATCTTCCCCACAGTGGATTG | ACGTTGGATGACTGCTGCAGGAAAGGACTT |
| 885743 | ACGTTGGATGTGAGAGAAGGCGATCTTGAC | ACGTTGGATGCCAATTCACAATCCACTGTG |
| 710845 | ACGTTGGATGAGGCCGAGAGCTCAGGCGAG | ACGTTGGATGTCGGGTCCGCCCTCCGCGCCT |
| 2569709 | ACGTTGGATGTATTCAACTCCAAGGGCGTC | ACGTTGGATGAGAAACAGAAGCGGAGACAG |
| 2569710 | ACGTTGGATGACCCCCATTTTCTACCCATC | ACGTTGGATGTTGCAGAAACAGAAGCGGAG |
| 2569711 | ACGTTGGATGTGTCTCCGCTTCTGTTTCTG | ACGTTGGATGTGAATAACTCGGGAGGTCAC |
| 2569712 | ACGTTGGATGACCCCAGGCCCTCTCAAAAA | ACGTTGGATGTCTGCAAGGTGGAACAAGCC |
| 12610026 | ACGTTGGATGCGGTGGCTCATGCGTGTAAT | ACGTTGGATGCTCCCGAATAGCTGGGACTA |
| 4804129 | ACGTTGGATGTAACACGGTGAAACCCCGTC | ACGTTGGATGTGAGTAGCTGGGACTACAGG |
| 12150978 | ACGTTGGATGAGACAGCAAGACTCCGTCTC | ACGTTGGATGCCCGCCTTAGTTTCCCAAAG |
| 439843 | ACGTTGGATGTGGCTAACATGGTGAAACCC | ACGTTGGATGTTGAGTAGCTGGGACTACAG |
| 892188 | ACGTTGGATGGTTTGTTTTTAGAGACAGGG | ACGTTGGATGGTCAAAGCCACTTCCAGCTA |
| 2291473 | ACGTTGGATGAAGAGGCTATGTGGCAGATG | ACGTTGGATGAGGGTGAAGCTGGGTTTAAC |
| 281416 | ACGTTGGATGTAACGTAGAGCACAGGTGAG | ACGTTGGATGCAACGCAAACACCAGTGTGG |
| 281417 | ACGTTGGATGAAGAGACAGTGGAGAGGCTG | ACGTTGGATGAGAGCCATCGGGTCCCAGCAA |
| 882589 | ACGTTGGATGTCGATAACCCCAGCAAAGAG | ACGTTGGATGTAAGGCCAAACCCCATTCCC |
| 1048941 | ACGTTGGATGAGATTGTGCTGACACCGGAG | ACGTTGGATGCCACGTAGAAGTTCCTGGTG |
| 281418 | ACGTTGGATGTGCGCTCAGTCAGCTTCCTC | ACGTTGGATGAGTGTTAGCCGAGGGCAAGC |
| 430092 | ACGTTGGATGGTTGGGATTACAGGCATGAG | ACGTTGGATGATCTGTTGCCTGTCAAGATG |
| 368835 | ACGTTGGATGGGTGGGAAAAAGACGTGAAG | ACGTTGGATGAGAGGGAATTAAGGAGGTCC |
| 2358583 | ACGTTGGATGAAGACGTGAAGAGACACACC | ACGTTGGATGAGAGGGAATTAAGGAGGTCC |
| 378395 | ACGTTGGATGACTTGGCCCCCTGCACTCACA | ACGTTGGATGACCGTGTTTTCCAGGCTCGCG |
| 395782 | ACGTTGGATGATGCATGTCATGGCCGCCTC | ACGTTGGATGTTCCACCAGGTGCCCCTGGCA |
| 1045384 | ACGTTGGATGTTCAACAAGCGAGTGACAGC | ACGTTGGATGGTGCAGAGATGGGCTTTCTC |
| 281427 | ACGTTGGATGGACAATTGTAGTACCCAGCC | ACGTTGGATGAGGAGAATCGCTTGAACCTG |
| 3745264 | ACGTTGGATGCAAAGTGCTAGGATCACAGG | ACGTTGGATGACTGCCCCATAGAGTGGCAA |
| 281426 | ACGTTGGATGATCCTCACACCTCAGTCTCC | ACGTTGGATGAATGAGACTCCGTCTCTACC |
| 281425 | ACGTTGGATGCCAGCGGCCCAGCTCACTC | ACGTTGGATGTCTGAGGCCAGAGCCTCGGA |
| 281424 | ACGTTGGATGACCTGTGTTTCTAGGTGTGC | ACGTTGGATGCATGCCTGGGAAAAAACTCC |
| 281423 | ACGTTGGATGAACCTCAAGCTGCTTCACTG | ACGTTGGATGGAGGAGCCCACCTTTAATGT |
| 281422 | ACGTTGGATGAGAAATCCTCCTACCTTGGC | ACGTTGGATGGCCCGGCCTCTACATAAAAT |
| 281420 | ACGTTGGATGCCAGGACTGTCTCTCTGTTT | ACGTTGGATGATGACACTACAGCCTGAGCA |
| 3745263 | ACGTTGGATGTACATGAAGAAGGACTCGGC | ACGTTGGATGATCCGTCCAGTGCACGTAGA |
| 3745262 | ACGTTGGATGGCAGCCGAGTCCTTCTTCAT | ACGTTGGATGTTCCTGGTCTACAGTGAGCG |
| 3745261 | ACGTTGGATGACACACAGCAGGGCATCCGT | ACGTTGGATGCGCAATCAATGCTTTCCACC |
| 3181049 | ACGTTGGATGATCTTCAGGGATGGTCACTC | ACGTTGGATGGACAAATACAAAGGGACAGG |
| 281412 | ACGTTGGATGTGACCTCAGGTGATTCACCC | ACGTTGGATGGGTATACCTTTAGCTGGCTG |
| 3181048 | ACGTTGGATGCTTTTCTCCACCCACTCTAC | ACGTTGGATGTAAACGCGTGATATGGAGGG |
| 2230399 | ACGTTGGATGAGCGGCAGTTACCATGTTAG | ACGTTGGATGTTCTTCCCCCATTGCTTCTG |
| 2278442 | ACGTTGGATGGGTGATGGACATTGAGGGTG | ACGTTGGATGTCCCTTCTGTCTCCAACCC |
| 3181047 | ACGTTGGATGGAATGCGACAGTCATGAACC | ACGTTGGATGCCCCTAGGGTCACGTTGCA |
| 3181046 | ACGTTGGATGTATCTTGCTGACTGGGTCAC | ACGTTGGATGCGTGGGATCTTTGAAGAAGG |
| 2304237 | ACGTTGGATGTGGGCCAGAACTTCACCCTG | ACGTTGGATGAAGCAGCACCACCGTGAGG |
| 281413 | ACGTTGGATGTCAAAGCTCACAGTTCTCGG | ACGTTGGATGACTTAGCGGGTCCTGCAAAC |
| 1058154 | ACGTTGGATGTCCCTTCCATCCTCATTTTT | ACGTTGGATGTGCAAGGCGCTAAACAAAAC |
| 3176769 | ACGTTGGATGTTCCTGTTTATGGCCAGACG | ACGTTGGATGGTCTGAACCTGATTGGAGAG |
| 2304238 | ACGTTGGATGCCTGGACATTTGTGTGTGAG | ACGTTGGATGCATGAGATTGAGATGCGTC |
| 2304239 | ACGTTGGATGCAGGCTCCTCTAACATCACC | ACGTTGGATGACACCACTACCCAAGACCAG |
| 2304240 | ACGTTGGATGAATCTCAGCAACGTGACTGG | ACGTTGGATGACACGGTGATGTTAGAGGAG |
| 3176768 | ACGTTGGATGGAGAGGTGTTAAATGGTGGG | ACGTTGGATGGGAACATGAAGAAGTCCTGG |
| 3176767 | ACGTTGGATGCGGTCTCTGATGGATTCTAC | ACGTTGGATGAACAGGCCCCACCATTTAAC |
| 3176766 | ACGTTGGATGTTTCGGGCTGCAATGGTCCC | ACGTTGGATGTAACACCTCTCTCCTTGTGC |
| 281414 | ACGTTGGATGAAGGCACCTTCCTCTGTCAG | ACGTTGGATGTGGGCCACAACACGGATGGTA |
| 281415 | ACGTTGGATGGCACAAAGAGCTAAGGTAGG | ACGTTGGATGGAATCCTGGATAGACAGTGG |

TABLE 11

| dbSNP rs# | Extend Primer | Term Mix |
|---|---|---|
| 2884487 | AGAGACAGGGTCTCGCC | ACT |
| 2358580 | TCATCCAGCGGCGCCTCGC | ACT |
| 2304236 | GCCCTGGGTAGCGTGGGG | ACT |
| 1059840 | GCTCAGAAGAGGTGCTTCAC | CGT |
| 1059843 | CTGTTGATGTGAAGCACCTCTT | ACG |
| 11115 | AAGGGTGGGCGTGGGCCT | ACT |
| 1059849 | CAGAAGCCCGTCTGGGCT | ACG |
| 1059855 | AAAAAGAGGGAATGGATGCA | ACT |
| 5030386 | CCTGGGCTCAAGTGATCCT | ACT |
| 5030339 | GCTGGGATTACAGGTGGGA | ACT |
| 5030387 | TTGTGGCTGCAAGTGGGAC | ACT |
| 5030388 | CGCGCCCCACAACAGGAAA | ACT |
| 1799766 | GCGGAGCCTGGGACGCC | CGT |
| 5030389 | GCGCCGAGCCCCTCCGC | ACT |
| 5490 | CTACTCAGAGTTGCAACCTC | CGT |
| 11575070 | CCTGCCTCAGCCTCCCGA | ACT |
| 5030340 | GCGTCTCTTACAGTTTCTCAG | ACG |
| 5030390 | CCAAAGCTGAGAAGTGGGAC | ACT |
| 5030391 | GGGGAACCAGGAGGGTGG | ACT |
| 3093035 | TTCTGTCTCAAAAAACAAAGC | ACT |
| 11667983 | CCTTCGGAGGCAGCAGAAT | ACG |
| 5030341 | TTTTTGAGACGGAGTCTCACT | ACT |
| 5030342 | GACGGAGTCTCACTCTGTC | ACT |
| 5030343 | GCTACTCAGGGAGGCTGAG | ACT |
| 5030344 | CTCCTCACTCCCTGAGACA | ACG |
| 5030347 | CTGCCTCCCGGGTTCAAGT | ACT |
| 5030348 | CACTTGAACCCGGGAGGCA | ACT |
| 5030349 | CAGACTGGAGTGCAGTGGC | ACT |
| 5030350 | TGCTAAAGATGTGTTCTTTTATTT | CGT |
| 5030351 | GAAGAAACGAGGGAAGAGGC | ACT |
| 5491 | CCTCCTGTGACCAGCCCA | CGT |
| 5030352 | CTGCACGTCTCTCCCACC | ACT |
| 5030353 | GGTGGGAGAGACGTGCAG | ACT |
| 10420063 | CGCCCGGCCATCAATTCTTT | ACG |
| 11879117 | CCCTCAGCCTCCCAAAGTG | ACT |
| 5030354 | TGTCATTTCTCCCACTTCCTT | ACT |
| 5030355 | CAGGAGGTGAAGGTTGCGG | ACT |
| 281428 | GCGCCCAGCACCACGCC | ACG |
| 5030358 | CAGGAGATCAAGACCAGCC | ACG |
| 5030359 | AGCCACTGCCCCCAGCC | ACT |
| 5030392 | GTCCCCAAAGTAGGAAGGAC | ACT |
| 5030393 | AGGTGGGAGGATCACCTGA | ACT |
| 5030360 | CTCCAGCCTGGGTGACAGA | ACT |
| 5030394 | CAGGTGAGGGAAGTCCCC | ACT |
| 281429 | GACTGTGTGTCAAAAAAAA | ACT |
| 5030361 | TATCAGGCAGAGCCGCGC | ACT |
| 5030362 | AGAGGGAGAAATGTGGCAGA | ACT |
| 281430 | GAGCCACTGCACCTGGCC | ACG |
| 281431 | ACAGGTGTGAGCCACTGC | ACT |
| 5030395 | GGCTCAAGTGGGAGGATCA | ACG |
| 5827095 | CCATCAATCAATTTTTTTTTTTTTTT | CGT |
| 281432 | CTAATCCCTGGCCTGCTCA | ACT |
| 5030364 | GGACCATAGCCAACTGCTC | CGT |
| 5030365 | GGTCAGAGTTTTCTTGACTATAT | ACT |
| 5030368 | GAGACCAGCCTGACCAACA | ACG |
| 5030369 | TCATCTGAGGTTGGGAGTTC | ACT |
| 3073809 | CGGCCTTTTTTTTTTTTTTTTTTTTT | ACT |
| 2358581 | CTTGCAGTGAGCCCAGATCG | CGT |
| 7258215 | CGTGTTAGCCAGGATGGTCT | ACG |
| 5030371 | AGCCTTGCTTTCTTGAGATAC | ACT |
| 5030372 | GGTTTCTTTTTTAGAGTGATTGA | ACT |
| 281433 | GTACATAAGCAGGAGGTTTCTT | ACT |
| 5030374 | CTCTGTCTACCTCTTAAGTGA | CGT |
| 5030375 | GTTCAGGAAACTGAGGTTCAG | ACT |
| 5030397 | CAAATGCAGGACCCCCCC | ACT |
| 281434 | TCAAGACCAGCCTGGCCAA | ACG |
| 12462944 | ATATTGGCCAGGCTGGTCTT | ACT |
| 5030398 | CAGGGTCTTTCTTTCCCAGG | ACT |
| 5030378 | CCTGGCCTCAAGTGACCCT | ACT |
| 12459133 | CTGGCCAACAGGTTTTTTTTTTT | ACT |
| 5030399 | CCCCCACCTCTGTTTTCCT | ACT |
| 5492 | CTGGCTCCCGTTTCAGCTC | ACT |
| 1800019 | CGTCCAGGGAACAGACCAC | ACT |
| 1799969 | CCGAGACTGGGAACAGCC | ACG |
| 5493 | GGTCTGTTCCCTGGACGG | ACT |
| 5030381 | AGGCCTCAGTCAGTGTGAC | ACG |
| 5494 | TCAGTGTGACCGCAGAGGA | ACG |
| 3093033 | GGGTTCAGGTCACACCC | ACG |
| 5495 | CTCTGGCTTCGTCAGAATCA | ACG |
| 1801714 | CCAGCCCAGCCACTGGGCC | ACG |
| 2071441 | CGGCCAGCTTATACACAAGAA | ACG |
| 5496 | CACCTCCATGTCATCTCATC | ACT |
| 5497 | GTTTTTCCAGATGGCCCCC | ACT |
| 5030382 | CAGAGCACATTCACGGTCACCT | ACG |
| 5030400 | TCCTCCCCACAGCCCCC | ACG |
| 2071440 | GAGGAAGAGGCCCTGTCC | ACG |
| 5499 | GCCATGCAGCTACACCTAC | ACG |
| 3093032 | CTTCTGTAAGTCTGTGGG | ACT |
| 1057981 | TACCTGGTGACCTTGAATGTGAT | ACG |
| 5500 | GAGTGCCTGGCAAAAAGATCA | ACT |
| 5501 | CCATATAGTGCTTTTGTGCCG | ACT |
| 5030383 | ACTCATGTCCAGACATGACC | ACT |
| 281436 | ACTGCAGTCTTGACCTTTTG | ACT |
| 923366 | TGCGAGACCCCGTCTCTG | ACT |
| 281437 | TTTTTTTTCCAGAGACGGGGTCT | ACG |
| 3093030 | CCAGAACCTCAGGGTATG | ACT |
| 5030384 | ATCACCGCCTACAGTGAGG | ACT |
| 5030385 | AGGGAGGTCTGTCGACCC | ACT |
| 3810159 | CCAAGTTCCTTGTCTCCCT | ACT |
| 281438 | CGAAGCCCCAGACTCTGTGTA | ACT |
| 3093029 | AGTTTCCTATCCCAGCC | ACT |
| 2735442 | GCTTTGACACCCCCCACC | ACT |
| 2569693 | CGCGTGGATGTCAGGGCC | ACG |
| 281439 | ACCCCTCCGGGTCAGCTCC | ACT |
| 281440 | TAATAAGCTGGACTCCGAGC | ACG |
| 2569694 | ATCCCCAAGCGCAACTCTG | ACG |
| 11575073 | CGGCCTCTCAGGTAAGAGC | ACG |
| 2569695 | CTCTGCCCTCGCCTCGCT | ACT |
| 2075741 | GTGGACCATGGTGCACAGCA | ACT |
| 11575074 | CTGTGCACCATGGTCCACG | ACT |
| 2569696 | TGTTCCCGCTCCACCCAGA | ACT |
| 2735439 | CAGCCTTGCGTCCCGGC | ACT |
| 2569697 | GGGACGTGGTAGCAGAGAG | ACT |
| 2075742 | AACCCAGGCGTCCCGCGTC | ACT |
| 2569698 | TGGGTTCTTTCCCTGGCTG | ACT |
| 11669397 | TTGCAGCACCAACTGCCCT | ACG |
| 901886 | AGAGTCCGCAGCTCTTTGAAC | ACT |
| 885742 | TGCACTATCTTATCGTATTATCA | ACG |
| 2569699 | CCCTCCGTGCCTTGAGGA | ACT |
| 11549918 | CCCAGGGTGACGTTGCAGA | ACG |
| 2569700 | TCAGTACCGGAACAGGCGTG | ACT |
| 2228615 | AGTAACCTGCGCAGCTGGG | ACT |
| ICAM-AA | TCCAAGGACCCGCCTGCT | ACG |
| 2569701 | CTTGAACCAGATAGAAATGCAC | ACG |
| 2569702 | CAGACCTTGAACCAGATAGAA | ACT |
| 2735440 | ACCAGTGGCGCCTTTTTCG | ACG |
| 2569703 | CTCCCAGACAGAGTGCATG | ACT |
| 10418913 | GCACCAGCGCTGGACAGC | ACT |
| 1056536 | GCAGCAGCACCCCCTCAGTGG | ACG |
| 2569704 | CTCCGGTGGCGCTGCAAA | ACT |
| 11673661 | GGACCACAGGCGCCCAC | ACT |
| 10402760 | CTCCTTCCCCGACAATCAG | ACT |
| | CACGTTGACCTGCCGCGC | ACT |
| 2569706 | GGCCGATGTTGAGGGCCC | ACG |
| 2569707 | GGCGAGTACGAGTGCGCA | ACT |
| 2436545 | ACGGTGCGCGTGGCCGGT | CGT |
| 2436546 | CCTCTCCCCAGCTGCCAC | ACT |
| 2916060 | CTCCCTCTCGGTCCCGG | ACT |
| 2916059 | CGGTCCCGGTAGACTAG | ACT |
| 2916058 | TTCTACTCTGTCCCACT | CGT |
| 2569708 | CTAGACGGAAGTGGGACAGA | ACT |
| 735747 | TGTGAATTGGGTCAAGTTTCC | ACT |
| 885743 | GACCCCTCTCTCCCTCCA | CGT |
| 710845 | AGGCGAGGCCGTGTGTCT | ACT |
| 2569709 | GGCGTCACCCCCATTTTCTA | ACT |
| 2569710 | TTTCTACCCATCCCCTCAATA | ACT |
| 2569711 | GTTTCTGCAAGGTGGAACAAG | ACT |
| 2569712 | GGCCCTCTCAAAAATCGCTT | CGT |
| 12610026 | AGCTGGGCATGGTGGTGC | ACT |
| 4804129 | AAACCCCGTCTCTACTAAAAAT | CGT |
| 12150978 | GCCAGGGCCGAGCACAGT | ACT |
| 439843 | ATGGTGAAACCCCGTCACTA | CGT |
| 892188 | TGGGCTGGAGCACAATGAC | ACT |
| 2291473 | GGAGTGTCCCTGGACCCC | ACT |
| 281416 | GGTCCACACCGACGCCAG | ACT |
| 281417 | CCCCTGCCCAGGACACCCC | ACT |
| 882589 | CCCCAGCAAAGAGAGGTCAT | ACT |
| 1048941 | GGAAGGAGCGGAATTCACCCT | ACT |
| 281418 | TCAGCTTCCTCCCTCCCC | ACT |
| 430092 | ATTACAGGCATGAGCCACTG | ACG |
| 368835 | AGACGTGAAGAGACACACCT | ACT |
| 2358583 | AAGAGACACACCTAATTTGTGG | ACT |
| 378395 | GCCCGCGTCCTCCTCTCC | CGT |
| 395782 | GCGTGAGTGCCAGGGTTCT | ACT |
| 1045384 | ACAATGTCCGACTCCCACA | ACT |
| 281427 | CTTTGTATACAATCTTCCCTC | ACG |
| 3745264 | ATACCATGCCAGGCATT | ACT |
| 281426 | GAGCTGGGACCACAGGCA | ACG |
| 281425 | CCAGCTCACTCCTCCCCC | CGT |
| 281424 | TAGGTGTGCGTGTGTGTGTG | ACG |
| 281423 | GCCCACCCTCCATTCAGC | ACG |
| 281422 | CTGGGGAACTACAGGAATGC | ACT |
| 281420 | ACTGTCTCTCTGTTTTTGAGAT | ACT |
| 3745263 | TCGGCTGCCCGTGCCAAGTC | ACT |
| 3745262 | GAGTCCTTCTTCATGTACTC | ACT |
| 3745261 | GCAGCTGCACCGACAGTTC | ACT |
| 3181049 | ACTCCCTGCCCTGGCCC | ACT |
| 281412 | GCTGGGATTATAAGCGTG | ACT |
| 3181048 | TACCACAGGGTGGCGGG | ACT |
| 2230399 | GTTACCATGTTAGGGAGGAGA | ACT |
| 2278442 | GGACATTGAGGGTGAGCTAA | ACG |
| 3181047 | CAGGAGGGTGCCCGGGA | ACT |
| 3181046 | ACTGGGTCACCCTTCTTC | ACT |
| 2304237 | TGCGCTGCCAAGTGGAGG | ACT |
| 281413 | GCTCACAGTTCTCGGCAGGAC | ACG |
| 1058154 | CTTCCATCCTCATTTTTTTTTATT | ACT |
| 3176769 | CGGGGTGGGTGGATCAA | ACT |
| 2304238 | CATTTGTGTGTGAGATACAAAGA | ACG |
| 2304239 | ATCACCGTGTACAGTGAGTC | ACT |
| 2304240 | GCTCAGTGTACTGCAATGGCTC | ACG |
| 3176768 | TGTTGATGCGTGGGTTGGGG | ACT |
| 3176767 | TGGATTCTACCTTTCCC | CGT |
| 3176766 | TCCTTCTGAGTTCTCCC | ACG |
| 281414 | CCTTCCTCTGTCAGAATGGC | ACG |
| 281415 | GGTGATTTGGGGACAGCTGA | ACT |

### Genetic Analysis of Allelotyping Results

Allelotyping results are shown for cases and controls in Table 12. The allele frequency for the A2 allele is noted in the fifth and sixth columns for breast cancer pools and control pools, respectively, where “AF” is allele frequency. SNPs with blank allele frequencies were untyped.

TABLE 12

| | | | | | | | | Breast |
|---|---|---|---|---|---|---|---|---|
| | Position in | | | | | | | Cancer |
| | SEQ ID | Chromosome | A1/A2 | | | | | Associated |
| dbSNP rs# | NO: 1 | Position | Allele | Case AF | Control AF | p-Value | OR | Allele |
| 2884487 | 230 | 10219830 | T/C | T = 0.788 | T = 0.76 | 0.307 | 0.85 | T |
| | | | | C = 0.212 | C = 0.240 | | | |
| 2358580 | 4282 | 10223882 | A/G | A = | A = 0.474 | | | |
| | | | | G = | G = 0.526 | | | |
| 2304236 | 11844 | 10231444 | A/C | A = 0.986 | A = 0.995 | 0.335 | 2.92 | C |
| | | | | C = 0.014 | C = 0.005 | | | |
| 1059840 | 11890 | 10231490 | A/T | A = 0.19 | A = 0.203 | 0.602 | 1.09 | T |
| | | | | T = 0.810 | T = 0.797 | | | |
| 1059843 | 11905 | 10231505 | C/T | C = 0.999 | C = 0.999 | 0.96 | 1.26 | T |
| | | | | T = 0.001 | T = 0.001 | | | |
| 11115 | 11942 | 10231542 | T/C | T = 0.566 | T = 0.634 | 0.0401 | 1.33 | C |
| | | | | C = 0.434 | C = 0.366 | | | |
| 1059849 | 12054 | 10231654 | G/A | G = 0.754 | G = 0.804 | 0.0649 | 1.34 | A |
| | | | | A = 0.246 | A = 0.196 | | | |
| 1059855 | 12070 | 10231670 | T/C | T = 0.001 | T = 0.003 | 0.838 | 3.25 | C |
| | | | | C = 0.999 | C = 0.997 | | | |
| 5030386 | 21497 | 10241097 | C/G | C = 0.999 | C = 0.999 | 0.987 | 1.12 | G |
| | | | | G = 0.001 | G = 0.001 | | | |
| 5030339 | 21537 | 10241137 | A/G | A = 0.002 | A = 0.002 | 0.993 | 0.93 | A |
| | | | | G = 0.998 | G = 0.998 | | | |
| 5030387 | 21682 | 10241282 | T/G | T = 0.999 | T = 0.995 | 0.706 | 0.27 | T |
| | | | | G = 0.001 | G = 0.005 | | | |
| 5030388 | 22073 | 10241673 | A/G | A = 0.001 | A = 0.001 | 0.968 | 3.66 | G |
| | | | | G = 0.999 | G = 0.999 | | | |
| 1799766 | 22393 | 10241993 | C/— | C = 0.001 | C = 0.002 | 0.952 | 1.46 | — |
| | | | | — = 0.999 | — = 0.998 | | | |
| 5030389 | 22394 | 10241994 | A/— | A = 0.983 | A = 0.979 | 0.757 | 0.80 | A |
| | | | | — = 0.017 | — = 0.021 | | | |
| 5490 | 23227 | 10242827 | C/A | C = 0.001 | C = 0.001 | 1 | 1.00 | |
| | | | | A = 0.999 | A = 0.999 | | | |
| 11575070 | 23614 | 10243214 | C/G | C = 0.001 | C = 0.001 | 0.986 | 1.58 | G |
| | | | | G = 0.999 | G = 0.999 | | | |
| 5030340 | 23681 | 10243281 | C/T | C = 0.963 | C = 0.967 | 0.829 | 1.13 | T |
| | | | | T = 0.037 | T = 0.033 | | | |
| 5030390 | 23937 | 10243537 | A/G | A = 0.191 | A = 0.145 | 0.0748 | 0.72 | A |
| | | | | G = 0.809 | G = 0.855 | | | |
| 5030391 | 24037 | 10243637 | A/G | A = 0.012 | A = 0.044 | 0.0373 | 3.95 | G |
| | | | | G = 0.988 | G = 0.956 | | | |
| 3093035 | 24373 | 10243973 | A/G | A = 0.113 | A = 0.085 | 0.223 | 0.73 | A |
| | | | | G = 0.887 | G = 0.915 | | | |
| 11667983 | 24589 | 10244189 | C/T | C = 1.000 | C = 0.998 | 0.84 | 0.36 | C |
| | | | | T = 0.000 | T = 0.002 | | | |
| 5030341 | 24675-24676 | 1024427\hat{ }10244276 | CT/— | CT = 0.691 | CT = 0.745 | 0.0746 | 1.30 | — |
| | | | | — = 0.309 | — = 0.255 | | | |
| 5030342 | 24680 | 10244280 | T/G | T = 0.373 | T = 0.389 | 0.634 | 1.07 | G |
| | | | | G = 0.627 | G = 0.611 | | | |
| 5030343 | 24754 | 10244354 | T/C | T = 0.674 | T = | | | |
| | | | | C = 0.326 | C = | | | |
| 5030344 | 25053 | 10244653 | G/A | G = 1.000 | G = 1.000 | 0.952 | 0.35 | G |
| | | | | A = 0.000 | A = 0.000 | | | |
| 5030347 | 26004 | 10245604 | A/C | A = 0 | A = 0 | 1 | | |
| | | | | C = 1.000 | C = 1.000 | | | |
| 5030348 | 26023 | 10245623 | A/G | A = 0.076 | A = 0.072 | 0.843 | 0.95 | A |
| | | | | G = 0.924 | G = 0.928 | | | |
| 5030349 | 26046 | 10245646 | T/C | T = 0.832 | T = 0.819 | 0.618 | 0.92 | T |
| | | | | C = 0.168 | C = 0.181 | | | |
| 5030350 | 26100 | 10245700 | A/T | A = 1.000 | A = 1.000 | 1 | | |
| | | | | T = 0 | T = 0 | | | |
| 5030351 | 26817 | 10246417 | T/C | T = 0.002 | T = 0.002 | 0.998 | 1.02 | 0 |
| | | | | C = 0.998 | C = 0.998 | | | |
| 5491 | 26940 | 10246540 | T/A | T = 0.001 | T = 0.002 | 0.853 | 3.82 | A |
| | | | | A = 0.999 | A = 0.998 | | | |
| 5030352 | 27143 | 10246743 | G/C | G = 0.631 | G = 0.62 | 0.732 | 0.96 | G |
| | | | | C = 0.369 | C = 0.380 | | | |
| 5030353 | 27162 | 10246762 | A/G | A = 0.002 | A = 0.004 | 0.858 | 2.12 | G |
| | | | | G = 0.998 | G = 0.996 | | | |
| 10420063 | 28328 | 10247928 | G/A | G = 0.001 | G = 0.002 | 0.864 | 6.56 | A |
| | | | | A = 0.999 | A = 0.998 | | | |
| 11879117 | 28799 | 10248399 | G/C | G = 0.998 | G = 1.000 | 0.844 | 3.03 | C |
| | | | | C = 0.002 | C = 0.000 | | | |
| 5030354 | 29166 | 10248766 | A/C | A = 0.999 | A = 1.000 | 0.989 | 1.15 | C |
| | | | | C = 0.001 | C = 0.000 | | | |
| 5030355 | 29331 | 10248931 | A/C | A = 0.999 | A = 0.999 | 0.985 | 1.12 | C |
| | | | | C = 0.001 | C = 0.001 | | | |
| 281428 | 29724 | 10249324 | C/T | C = 0.821 | C = 0.823 | 0.951 | 1.01 | T |
| | | | | T = 0.179 | T = 0.177 | | | |
| 5030358 | 29773 | 10249373 | G/A | G = 0.006 | G = 0.007 | 0.966 | 1.12 | A |
| | | | | A = 0.994 | A = 0.993 | | | |
| 5030359 | 29862 | 10249462 | A/G | A = 0.001 | A = 0.003 | 0.858 | 4.75 | G |
| | | | | G = 0.999 | G = 0.997 | | | |
| 5030392 | 29877 | 10249477 | T/C | T = 0.002 | T = 0.001 | 0.958 | 0.59 | T |
| | | | | C = 0.998 | C = 0.999 | | | |
| 5030393 | 30194 | 10249794 | T/G | T = 0 | T = 0.005 | 0.836 | | G |
| | | | | G = 1.000 | G = 0.995 | | | |
| 5030360 | 30258 | 10249858 | T/G | T = 0 | T = 0.001 | 0.972 | | G |
| | | | | G = 1.000 | G = 0.999 | | | |
| 5030394 | 30549 | 10250149 | A/G | A = 0.005 | A = 0.006 | 0.926 | 1.23 | G |
| | | | | G = 0.995 | G = 0.994 | | | |
| 281429 | 30648 | 10250248 | T/C | T = 0.999 | T = 1.000 | 0.938 | 2.63 | C |
| | | | | C = 0.001 | C = 0.000 | | | |
| 5030361 | 30909 | 10250509 | T/C | T = 0.002 | T = 0.002 | 0.989 | 1.10 | C |
| | | | | C = 0.998 | C = 0.998 | | | |
| 5030362 | 30962 | 10250562 | A/C | A = 0.004 | A = 0.002 | 0.791 | 0.29 | A |
| | | | | C = 0.996 | C = 0.998 | | | |
| 281430 | 31337 | 10250937 | G/A | G = 0.015 | G = 0.008 | 0.498 | 0.51 | G |
| | | | | A = 0.985 | A = 0.992 | | | |
| 281431 | 31345 | 10250945 | T/C | T = | T = 0.886 | | | T |
| | | | | C = | C = 0.114 | | | |
| 5030395 | 31534 | 10251134 | C/T | C = 0.998 | C = 0.998 | 0.973 | 1.11 | T |
| | | | | T = 0.002 | T = 0.002 | | | |
| 5827095 | 31942 | 10251542 | A/— | A = 0.532 | A = 0.532 | 0.986 | 1.00 | A |
| | | | | — = 0.468 | — = 0.468 | | | |
| 281432 | 32058 | 10251658 | G/C | G = 0.51 | G = 0.553 | 0.194 | 1.19 | C |
| | | | | C = 0.490 | C = 0.447 | | | |
| 5030364 | 32109 | 10251709 | A/T | A = 0.001 | A = 0.002 | 0.902 | 4.40 | T |
| | | | | T = 0.999 | T = 0.998 | | | |
| 5030365 | 32401 | 10252001 | A/G | A = 1.000 | A = 1.000 | 1 | 1.03 | |
| | | | | G = 0.000 | G = 0.000 | | | |
| 5030368 | 32845 | 10252445 | G/A | G = 0.099 | G = 0.097 | 0.901 | 0.97 | G |
| | | | | A = 0.901 | A = 0.903 | | | |
| 5030369 | 32864 | 10252464 | T/C | T = 0.506 | T = 0.487 | 0.541 | 0.92 | T |
| | | | | C = 0.494 | C = 0.513 | | | |
| 3073809 | 32965-32966 | 1025256\hat{ }10252566 | TT/— | T = 0.848 | T = 0.859 | 0.69 | 1.10 | — |
| | | | | — = 0.152 | — = 0.141 | | | |
| 2358581 | 33011 | 10252611 | G/T | G = 0.904 | G = 0.919 | 0.525 | 1.20 | T |
| | | | | T = 0.096 | T = 0.081 | | | |
| 7258215 | 33170 | 10252770 | C/T | C = 0.85 | C = 0.847 | 0.88 | 0.97 | C |
| | | | | T = 0.150 | T = 0.153 | | | |
| 5030371 | 33516 | 10253116 | A/G | A = 0.001 | A = 0.001 | 0.969 | 0.58 | A |
| | | | | G = 0.999 | G = 0.999 | | | |
| 5030372 | 33608 | 10253208 | T/C | T = 1.000 | T = 1.000 | 0.997 | 1.08 | C |
| | | | | C = 0.000 | C = 0.000 | | | |
| 281433 | 33623 | 10253223 | A/C | A = 1.000 | A = 1.000 | 0.989 | 1.34 | C |
| | | | | C = 0.000 | C = 0.000 | | | |
| 5030374 | 33672 | 10253272 | C/A | C = 0.156 | C = | | | A |
| | | | | A = 0.844 | A = | | | |
| 5030375 | 33724 | 10253324 | A/— | A = 0.999 | A = 1.000 | 0.94 | 2.17 | — |
| | | | | — = 0.001 | — = 0.000 | | | |
| 5030397 | 33787 | 10253387 | T/G | T = 0.231 | T = 0.18 | 0.0697 | 0.73 | T |
| | | | | G = 0.769 | G = 0.820 | | | |
| 281434 | 34020 | 10253620 | G/A | G = 0.932 | G = 0.93 | 0.902 | 0.97 | G |
| | | | | A = 0.068 | A = 0.070 | | | |
| 12462944 | 34038 | 10253638 | C/G | C = 0.004 | C = 0.006 | 0.888 | 1.32 | G |
| | | | | G = 0.996 | G = 0.994 | | | |
| 5030398 | 34190 | 10253790 | C/G | C = 0.959 | C = 0.993 | 0.064 | 5.64 | G |
| | | | | G = 0.041 | G = 0.007 | | | |
| 5030378 | 34706 | 10254306 | C/G | C = 0.993 | C = 0.995 | 0.918 | 1.25 | G |
| | | | | G = 0.007 | G = 0.005 | | | |
| 12459133 | 34778 | 10254378 | T/G | T = 0.376 | T = 0.347 | 0.365 | 0.88 | T |
| | | | | G = 0.624 | G = 0.653 | | | |
| 5030399 | 35553 | 10255153 | A/G | A = 0.002 | A = 0.001 | 0.922 | 0.23 | A |
| | | | | G = 0.998 | G = 0.999 | | | |
| 5492 | 35690 | 10255290 | G/C | G = 1.000 | G = 1.000 | 0.993 | 0.00 | G |
| | | | | C = 0 | C = 0.000 | | | |
| 1800019 | 36173 | 10255773 | A/C | A = 0.002 | A = 0.001 | 0.914 | 0.47 | A |
| | | | | C = 0.998 | C = 0.999 | | | |
| 1799969 | 36192 | 10255792 | G/A | G = 0.882 | G = 0.849 | 0.134 | 0.75 | G |
| | | | | A = 0.118 | A = 0.151 | | | |
| 5493 | 36194 | 10255794 | T/G | T = 0.001 | T = 0 | 0.99 | 0.00 | T |
| | | | | G = 0.999 | G = 1.000 | | | |
| 5030381 | 36308 | 10255908 | C/T | C = 0.877 | C = | | | |
| | | | | T = 0.123 | T = | | | |
| 5494 | 36317 | 10255917 | C/T | C = 0.999 | C = 1.000 | 0.873 | 5.90 | T |
| | | | | T = 0.001 | T = 0.000 | | | |
| 3093033 | 36431 | 10256031 | G/A | G = 0.991 | G = 0.969 | 0.113 | 0.30 | G |
| | | | | A = 0.009 | A = 0.031 | | | |
| 5495 | 36496 | 10256096 | G/A | G = 1.000 | G = 0.999 | 0.888 | 0.19 | G |
| | | | | A = 0.000 | A = 0.001 | | | |
| 1801714 | 36608 | 10256208 | C/T | C = 0.995 | C = 0.98 | 0.129 | 0.25 | C |
| | | | | T = 0.005 | T = 0.020 | | | |
| 2071441 | 36708 | 10256308 | C/T | C = 1.000 | C = 1.000 | 0.993 | 1.25 | T |
| | | | | T = 0.000 | T = 0.000 | | | |
| 5496 | 36847 | 10256447 | A/G | A = 0.003 | A = 0.009 | 0.469 | 4.06 | G |
| | | | | G = 0.997 | G = 0.991 | | | |
| 5497 | 36868 | 10256468 | A/G | A = 0.004 | A = 0.006 | 0.813 | 1.61 | G |
| | | | | G = 0.996 | G = 0.994 | | | |
| 5030382 | 37083 | 10256683 | G/A | G = 0.449 | G = 0.512 | 0.0484 | 1.29 | A |
| | | | | A = 0.551 | A = 0.488 | | | |
| 5030400 | 37196 | 10256796 | C/T | C = 1.000 | C = 0.999 | 0.969 | 0.76 | C |
| | | | | T = 0.000 | T = 0.001 | | | |
| 2071440 | 37373 | 10256973 | G/A | G = 0.999 | G = 1.000 | 0.875 | 5.74 | A |
| | | | | A = 0.001 | A = 0.000 | | | |
| 5499 | 37465 | 10257065 | C/T | C = 0.994 | C = 1.000 | 0.47 | 8.13 | T |
| | | | | T = 0.006 | T = 0.000 | | | |
| 3093032 | 37736 | 10257336 | T/C | T = 0.322 | T = 0.267 | 0.0662 | 0.77 | T |
| | | | | C = 0.678 | C = 0.733 | | | |
| 1057981 | 37959 | 10257559 | G/A | G = 0.032 | G = 0.026 | 0.693 | 0.80 | G |
| | | | | A = 0.968 | A = 0.974 | | | |
| 5500 | 38031 | 10257631 | A/G | A = 0.995 | A = 0.996 | 0.868 | 1.35 | G |
| | | | | G = 0.005 | G = 0.004 | | | |
| 5501 | 38091 | 10257691 | T/C | T = 1.000 | T = 0.999 | 0.862 | 0.06 | T |
| | | | | C = 0.000 | C = 0.001 | | | |
| 5030383 | 38243 | 10257843 | T/C | T = 0.002 | T = 0.001 | 0.964 | 0.58 | T |
| | | | | C = 0.998 | C = 0.999 | | | |
| 281436 | 38531 | 10258131 | A/G | A = 0.501 | A = 0.452 | 0.132 | 0.82 | A |
| | | | | G = 0.499 | G = 0.548 | | | |
| 923366 | 38623 | 10258223 | T/C | T = 0.517 | T = 0.562 | 0.161 | 1.20 | C |
| | | | | C = 0.483 | C = 0.438 | | | |
| 281437 | 38638 | 10258238 | C/T | C = 0.804 | C = 0.852 | 0.0793 | 1.39 | T |
| | | | | T = 0.196 | T = 0.148 | | | |
| 3093030 | 38803 | 10258403 | T/C | T = 0.553 | T = 0.606 | 0.137 | 1.24 | C |
| | | | | C = 0.447 | C = 0.394 | | | |
| 5030384 | 39489 | 10259089 | A/G | A = 0.001 | A = 0.001 | 0.988 | 0.80 | A |
| | | | | G = 0.999 | G = 0.999 | | | |
| 5030385 | 40008 | 10259608 | G/C | G = 0.002 | G = 0.002 | 0.974 | 0.79 | G |
| | | | | C = 0.998 | C = 0.998 | | | |
| 3810159 | 40711 | 10260311 | T/C | T = 0.001 | T = 0.001 | 0.915 | 68.31 | C |
| | | | | C = 0.999 | C = 0.999 | | | |
| 281438 | 40775 | 10260375 | T/G | T = 0.767 | T = 0.802 | 0.31 | 1.23 | G |
| | | | | G = 0.233 | G = 0.198 | | | |
| 3093029 | 40951 | 10260551 | C/G | C = 0.912 | C = 0.92 | 0.683 | 1.11 | G |
| | | | | G = 0.088 | G = 0.080 | | | |
| 2735442 | 41218 | 10260818 | G/C | G = 0.999 | G = 0.999 | 0.97 | 0.79 | G |
| | | | | C = 0.001 | C = 0.001 | | | |
| 2569693 | 41304 | 10260904 | C/T | C = 0.703 | C = 0.641 | 0.0479 | 0.75 | C |
| | | | | T = 0.297 | T = 0.359 | | | |
| 281439 | 41510 | 10261110 | G/C | G = 0.471 | G = 0.418 | 0.111 | 0.81 | G |
| | | | | C = 0.529 | C = 0.582 | | | |
| 281440 | 41704 | 10261304 | G/A | G = 0.268 | G = 0.255 | 0.676 | 0.94 | G |
| | | | | A = 0.732 | A = 0.745 | | | |
| 2569694 | 42020 | 10261620 | C/T | C = 0.999 | C = 0.999 | 0.972 | 1.20 | T |
| | | | | T = 0.001 | T = 0.001 | | | |
| 11575073 | 42210 | 10261810 | C/T | C = 0.924 | C = | | | T |
| | | | | T = 0.076 | T = | | | |
| 2569695 | 42302 | 10261902 | C/G | C = 1.000 | C = 0.998 | 0.815 | 0.02 | C |
| | | | | G = 0.000 | G = 0.002 | | | |
| 2075741 | 42498 | 10262098 | G/C | G = 0.715 | G = 0.631 | 0.0112 | 0.68 | G |
| | | | | C = 0.285 | C = 0.369 | | | |
| 11575074 | 42520 | 10262120 | A/G | A = 0.088 | A = 0.097 | 0.702 | 1.11 | G |
| | | | | G = 0.912 | G = 0.903 | | | |
| 2569696 | 42607 | 10262207 | A/G | A = 0.005 | A = 0.004 | 0.909 | 0.69 | A |
| | | | | G = 0.995 | G = 0.996 | | | |
| 2735439 | 42674 | 10262274 | T/G | T = 1.000 | T = 1.000 | 0.928 | | G |
| | | | | G = 0.000 | G = 0 | | | |
| 2569697 | 42929 | 10262529 | G/C | G = 1.000 | G = 1.000 | 0.97 | 3.84 | C |
| | | | | C = 0.000 | C = 0.000 | | | |
| 2075742 | 43007 | 10262607 | G/C | G = | G = 0.638 | | | G |
| | | | | C = | C = 0.362 | | | |
| 2569698 | 43040 | 10262640 | C/G | C = 1.000 | C = 1.000 | 0.98 | | G |
| | | | | G = 0.000 | G = 0 | | | |
| 11669397 | 43246 | 10262846 | C/T | C = 1.000 | C = 1.000 | 0.962 | 0.00 | C |
| | | | | T = 0 | T = 0.000 | | | |
| 901886 | 43531 | 10263131 | T/C | T = 0.667 | T = 0.645 | 0.544 | 0.90 | T |
| | | | | C = 0.333 | C = 0.355 | | | |
| 885742 | 44023 | 10263623 | G/A | G = 0.012 | G = 0.016 | 0.633 | 1.46 | A |
| | | | | A = 0.988 | A = 0.984 | | | |
| 2569699 | 44024 | 10263624 | T/G | T = 0.999 | T = 1.000 | 0.962 | 1.82 | G |
| | | | | G = 0.001 | G = 0.000 | | | |
| 11549918 | 44338 | 10263938 | C/T | C = 0.635 | C = 0.562 | 0.0293 | 0.74 | C |
| | | | | T = 0.365 | T = 0.438 | | | |
| 2569700 | 44499 | 10264099 | T/G | T = 0.006 | T = 0.011 | 0.703 | 1.80 | G |
| | | | | G = 0.994 | G = 0.989 | | | |
| 2228615 | 44768 | 10264368 | A/G | A = 0.402 | A = 0.484 | 0.0318 | 1.40 | G |
| | | | | G = 0.598 | G = 0.516 | | | |
| ICAM-AA | 45003 | 10264603 | C/T | C = 0.934 | C = 0.958 | 0.309 | 1.59 | T |
| | | | | T = 0.066 | T = 0.042 | | | |
| 2569701 | 45341 | 10264941 | G/A | G = 1.000 | G = 1.000 | 0.917 | 10.14 | A |
| | | | | A = 0.000 | A = 0.000 | | | |
| 2569702 | 45347 | 10264947 | T/C | T = 0.709 | T = 0.646 | 0.0681 | 0.75 | T |
| | | | | C = 0.291 | C = 0.354 | | | |
| 2735440 | 45577 | 10265177 | G/A | G = 0.01 | G = 0.006 | 0.624 | 0.52 | G |
| | | | | A = 0.990 | A = 0.994 | | | |
| 2569703 | 45627 | 10265227 | C/G | C = 0.563 | C = 0.527 | 0.274 | 0.86 | C |
| | | | | G = 0.437 | G = 0.473 | | | |
| 10418913 | 45794 | 10265394 | A/G | A = 0.001 | A = 0.002 | 0.923 | 2.09 | G |
| | | | | G = 0.999 | G = 0.998 | | | |
| 1056536 | 46244 | 10265844 | C/T | C = 1.000 | C = 1.000 | 0.989 | 2.49 | T |
| | | | | T = 0.000 | T = 0.000 | | | |
| 2569704 | 46469 | 10266069 | T/C | T = 0.003 | T = 0.001 | 0.905 | 0.24 | T |
| | | | | C = 0.997 | C = 0.999 | | | |
| 11673661 | 46999 | 10266599 | G/C | G = 0.997 | G = 0.999 | 0.874 | 2.39 | C |
| | | | | C = 0.003 | C = 0.001 | | | |
| 10402760 | 47265 | 10266865 | T/C | T = 0.168 | T = 0.104 | 0.0276 | 0.58 | T |
| | | | | C = 0.832 | C = 0.896 | | | |
| ICAM-AB | 47504 | 10267104 | A/G | A = 0.264 | A = | | | |
| | | | | G = 0.736 | G = | | | |
| 2569706 | 47547 | 10267147 | G/A | G = 0.997 | G = 1.000 | 0.666 | 45.64 | A |
| | | | | A = 0.003 | A = 0.000 | | | |
| 2569707 | 47637 | 10267237 | C/G | C = 0.173 | C = 0.171 | 0.942 | 0.99 | C |
| | | | | G = 0.827 | G = 0.829 | | | |
| 2436545 | 47691 | 10267291 | C/A | C = 0.001 | C = 0.001 | 0.991 | 0.86 | C |
| | | | | A = 0.999 | A = 0.999 | | | |
| 2436546 | 47692 | 10267292 | A/C | A = 1.000 | A = 0.998 | 0.866 | 0.26 | A |
| | | | | C = 0.000 | C = 0.002 | | | |
| 2916060 | 47788 | 10267388 | A/C | A = 0.99 | A = 0.993 | 0.803 | 1.39 | C |
| | | | | C = 0.010 | C = 0.007 | | | |
| 2916059 | 47796 | 10267396 | T/C | T = 0.999 | T = 0.999 | 0.968 | 0.73 | T |
| | | | | C = 0.001 | C = 0.001 | | | |
| 2916058 | 47800 | 10267400 | T/A | T = 1.000 | T = 0.999 | 0.869 | 0.30 | T |
| | | | | A = 0.000 | A = 0.001 | | | |
| 2569708 | 47812 | 10267412 | A/G | A = 0.004 | A = 0.003 | 0.936 | 0.73 | A |
| | | | | G = 0.996 | G = 0.997 | | | |
| 735747 | 47969 | 10267569 | T/C | T = 0.999 | T = 1.000 | 0.91 | 2.15 | C |
| | | | | C = 0.001 | C = 0.000 | | | |
| 885743 | 48035 | 10267635 | A/T | A = 0.661 | A = 0.676 | 0.698 | 1.07 | T |
| | | | | T = 0.339 | T = 0.324 | | | |
| 710845 | 48569 | 10268169 | C/G | C = 0.744 | C = 0.665 | 0.0156 | 0.68 | C |
| | | | | G = 0.256 | G = 0.335 | | | |
| 2569709 | 48823 | 10268423 | C/G | C = 0.999 | C = 1.000 | 0.967 | 1.45 | G |
| | | | | G = 0.001 | G = 0.000 | | | |
| 2569710 | 48838 | 10268438 | A/G | A = 0.999 | A = 0.998 | 0.943 | 0.71 | A |
| | | | | G = 0.001 | G = 0.002 | | | |
| 2569711 | 48894 | 10268494 | C/G | C = 1.000 | C = 1.000 | 0.98 | 0.60 | C |
| | | | | G = 0.000 | G = 0.000 | | | |
| 2569712 | 48934 | 10268534 | A/T | A = 1.000 | A = 1.000 | 0.983 | 1.93 | T |
| | | | | T = 0.000 | T = 0.000 | | | |
| 12610026 | 49249 | 10268849 | A/G | A = 0.992 | A = | | | G |
| | | | | G = 0.008 | G = | | | |
| 4804129 | 49509 | 10269109 | C/A | C = 0.002 | C = 0.002 | 0.978 | 0.83 | C |
| | | | | A = 0.998 | A = 0.998 | | | |
| 12150978 | 49718 | 10269318 | A/G | A = 0.8 | A = 0.782 | 0.599 | 0.90 | A |
| | | | | G = 0.200 | G = 0.218 | | | |
| 439843 | 49963 | 10269563 | C/A | C = 1.000 | C = 0.999 | 0.981 | 0.82 | C |
| | | | | A = 0.000 | A = 0.001 | | | |
| 892188 | 51193 | 10270793 | T/C | T = 0.487 | T = 0.567 | 0.0122 | 1.38 | C |
| | | | | C = 0.513 | C = 0.433 | | | |
| 2291473 | 57181 | 10276781 | T/C | T = 0.913 | T = 0.909 | 0.83 | 0.95 | T |
| | | | | C = 0.087 | C = 0.091 | | | |
| 281416 | 60184 | 10279784 | A/G | A = 0.456 | A = 0.496 | 0.221 | 1.18 | G |
| | | | | G = 0.544 | G = 0.504 | | | |
| 281417 | 60530 | 10280130 | T/C | T = 0.532 | T = 0.526 | 0.86 | 0.98 | T |
| | | | | C = 0.468 | C = 0.474 | | | |
| 882589 | 61354 | 10280954 | T/C | T = 0 | T = 0.002 | 0.865 | | C |
| | | | | C = 1.000 | C = 0.998 | | | |
| 1048941 | 62616 | 10282216 | T/G | T = 0.304 | T = | | | |
| | | | | G = 0.696 | G = | | | |
| 281418 | 62785 | 10282385 | G/C | G = 0.088 | G = 0.0680 | 0.369 | 0.76 | G |
| | | | | C = 0.912 | C = 0.932 | | | |
| 430092 | 66351 | 10285951 | C/T | C = 0.764 | C = 0.744 | 0.588 | 0.90 | C |
| | | | | T = 0.236 | T = 0.256 | | | |
| 368835 | 67386 | 10286986 | A/G | A = 0.303 | A = 0.274 | 0.347 | 0.86 | A |
| | | | | G = 0.697 | G = 0.726 | | | |
| 2358583 | 67395 | 10286995 | T/G | T = 0.696 | T = 0.674 | 0.505 | 0.90 | T |
| | | | | G = 0.304 | G = 0.326 | | | |
| 378395 | 67822 | 10287422 | C/A | C = 0.323 | C= | | | A |
| | | | | A = 0.677 | A= | | | |
| 395782 | 68028 | 10287628 | T/C | T = 0.726 | T = 0.656 | 0.115 | 0.72 | T |
| | | | | C = 0.274 | C = 0.344 | | | |
| 1045384 | 68646 | 10288246 | T/G | T = 0.326 | T = 0.302 | 0.424 | 0.89 | T |
| | | | | G = 0.674 | G = 0.698 | | | |
| 281427 | 70520 | 10290120 | C/T | C = 0.785 | C = 0.823 | 0.226 | 1.27 | T |
| | | | | T = 0.215 | T = 0.177 | | | |
| 3745264 | 70966 | 10290566 | T/G | T = 0.156 | T = 0.164 | 0.76 | 1.06 | G |
| | | | | G = 0.844 | G = 0.836 | | | |
| 281426 | 72451 | 10292051 | G/A | G = 0.437 | G = 0.321 | 0.000196 | 0.61 | G |
| | | | | A = 0.563 | A = 0.679 | | | |
| 281425 | 73199 | 10292799 | G/T | G = 0.806 | G = | | | |
| | | | | T = 0.194 | T = | | | |
| 281424 | 74319 | 10293919 | C/T | C = 0.753 | C = 0.748 | 0.861 | 0.97 | C |
| | | | | T = 0.247 | T = 0.252 | | | |
| 281423 | 76893 | 10296493 | C/T | C = 0.804 | C = 0.804 | 0.993 | 1.00 | T |
| | | | | T = 0.196 | T = 0.196 | | | |
| 281422 | 77755 | 10297355 | T/C | T = 0.372 | T = 0.364 | 0.829 | 0.97 | T |
| | | | | C = 0.628 | C = 0.636 | | | |
| 281420 | 79354 | 10298954 | A/G | A = 0.608 | A = 0.567 | 0.213 | 0.85 | A |
| | | | | G = 0.392 | G = 0.433 | | | |
| 3745263 | 80901 | 10300501 | A/G | A = 0.064 | A = 0.08 | 0.513 | 1.27 | G |
| | | | | G = 0.936 | G = 0.920 | | | |
| 3745262 | 80940 | 10300540 | T/C | T = 0.996 | T = 0.999 | 0.731 | 4.38 | C |
| | | | | C = 0.004 | C = 0.001 | | | |
| 3745261 | 81111 | 10300711 | T/C | T = 0.987 | T = 0.983 | 0.752 | 0.76 | T |
| | | | | C = 0.013 | C = 0.017 | | | |
| 3181049 | 82517 | 10302117 | T/C | T = 0.353 | T = 0.363 | 0.761 | 1.04 | C |
| | | | | C = 0.647 | C = 0.637 | | | |
| 281412 | 82874 | 10302474 | T/C | T = 0.846 | T = 0.841 | 0.823 | 0.96 | T |
| | | | | C = 0.154 | C = 0.159 | | | |
| 3181048 | 84978 | 10304578 | A/G | A = 1.000 | A = 1.000 | 0.98 | 0.46 | A |
| | | | | G = 0.000 | G = 0.000 | | | |
| 2230399 | 86003 | 10305603 | C/G | C = 0.171 | C = 0.161 | 0.708 | 0.93 | C |
| | | | | G = 0.829 | G = 0.839 | | | |
| 2278442 | 86226 | 10305826 | G/A | G = 0.42 | G = 0.405 | 0.674 | 0.94 | G |
| | | | | A = 0.580 | A = 0.595 | | | |
| 3181047 | 87206 | 10306806 | A/G | A = 0.008 | A = 0.003 | 0.654 | 0.30 | A |
| | | | | G = 0.992 | G = 0.997 | | | |
| 3181046 | 87535 | 10307135 | T/C | T = 0.999 | T = 1.000 | 0.946 | 2.38 | C |
| | | | | C = 0.001 | C = 0.000 | | | |
| 2304237 | 87968 | 10307568 | T/C | T = 0.898 | T = 0.907 | 0.726 | 1.10 | C |
| | | | | C = 0.102 | C = 0.093 | | | |
| 281413 | 88134 | 10307734 | G/A | G = 0.697 | G = 0.702 | 0.877 | 1.03 | A |
| | | | | A = 0.303 | A = 0.298 | | | |
| 1058154 | 88297 | 10307897 | A/C | A = 0.22 | A = 0.186 | 0.31 | 0.81 | A |
| | | | | C = 0.780 | C = 0.814 | | | |
| 3176769 | 88434 | 10308034 | T/C | T = 0.801 | T = 0.786 | 0.593 | 0.91 | T |
| | | | | C = 0.199 | C = 0.214 | | | |
| 2304238 | 90602 | 10310202 | G/A | G = 0.002 | G = 0.001 | 0.954 | 0.62 | G |
| | | | | A = 0.998 | A = 0.999 | | | |
| 2304239 | 90750 | 10310350 | T/C | T = 0.001 | T = 0.003 | 0.876 | 2.50 | C |
| | | | | C = 0.999 | C = 0.997 | | | |
| 2304240 | 90792 | 10310392 | G/A | G = 0.828 | G = 0.797 | 0.261 | 0.82 | G |
| | | | | A = 0.172 | A = 0.203 | | | |
| 3176768 | 91065 | 10310665 | A/G | A = 0.355 | A = 0.35 | 0.871 | 0.98 | A |
| | | | | G = 0.645 | G = 0.650 | | | |
| 3176767 | 91151 | 10310751 | C/A | C = 0.278 | C = 0.277 | 0.951 | 0.99 | C |
| | | | | A = 0.722 | A = 0.723 | | | |
| 3176766 | 91178 | 10310778 | C/T | C = 0.768 | C = 0.765 | 0.937 | 0.99 | C |
| | | | | T = 0.232 | T = 0.235 | | | |
| 281414 | 91685 | 10311285 | G/A | G = 0.697 | G = 0.72 | 0.47 | 1.12 | A |
| | | | | A = 0.303 | A = 0.280 | | | |
| 281415 | 92393 | 10311993 | T/G | T = 0.341 | T = 0.312 | 0.343 | 0.88 | T |
| | | | | G = 0.659 | G = 0.688 | | | |

FIG. 1A shows proximal SNPs in and around the ICAM region for females. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1A can be determined by consulting Table 12. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Web at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

Additional Genotyping

In addition to the ICAM region incident SNP, fourteen other SNPs were genotyped in the discovery cohort. The discovery cohort is described in Example 1. The SNP rs2228615 is located in the ICAM5 encoding portion of the sequence, and is associated with breast cancer with a p-value of 0.00236, and encoded non-synonymous amino acids (see Table 15).

The methods used to verify and genotype the two proximal SNPs of Table 15 are the same methods described in Examples 1 and 2 herein. The PCR primers and extend primers used in these assays are provided in Table 13 (SEQ ID NOS 634-663, respectively, in order of appearance) and Table 14 (SEQ ID NOS 664-678, respectively, in order of appearance), respectively.

TABLE 13

| | | |
|---|---|---|
| dbSNP rs# | Forward PCR primer | Reverse PCR primer |

| | | |
|---|---|---|
| 1801714 | ACGTTGGATGAGGGTTGCAGAGCAGGAGAA | ACGTTGGATGAGCCAAGGTGACGCTGAATG |
| 2228615 | ACGTTGGATGAGATGGTGACAGTAACCTGC | ACGTTGGATGTGGCATTTAGCTGAAGCTGG |
| 2569703 | ACGTTGGATGATAACCCTTGGACGCCGATC | ACGTTGGATGTTAGACGAAAAAGGCGCCAC |
| 1799969 | ACGTTGGATGAACCTCTGGTCCCCCAGTGC | ACGTTGGATGTCCTAGAGGTGGACACGCAG |
| 1059849 | ACGTTGGATGAAGCTGAGGCCACAGGGAG | ACGTTGGATGAGAGGGAATGGATGCAGAAG |
| 3093035 | ACGTTGGATGTAGAAAGCAGTGCGATCTGG | ACGTTGGATGGGAGACATAGCGAGATTCTG |
| 281439 | ACGTTGGATGGCTCGCTGGCACTTTCGTC | ACGTTGGATGGAGCCATACCTCTAAGCACC |
| 281432 | ACGTTGGATGAGCTGGGACTTTCCTTCTTG | ACGTTGGATGGCCTTCAGCTATCTAATCCC |
| 901886 | ACGTTGGATGTACGCGATCTGGTCGCTCTG | ACGTTGGATGTTCAGGCCCCACCTTCTGTT |
| 5030382 | ACGTTGGATGACTCACAGAGCACATTCACG | ACGTTGGATGAGATCTTGAGGGCACCTACC |
| 2569693 | ACGTTGGATGGTGTCTACTCAGCGTGTGTG | ACGTTGGATGCTTGTTCTCGCGTGGATGTC |
| 2569702 | ACGTTGGATGACCCTCCAGACCTTGAACCA | ACGTTGGATGGTCAAGGACGTAACGCTAAC |
| 3093030 | ACGTTGGATGACTTCCTCCCCCAAGAAAAC | ACGTTGGATGAGAGACCCAGAAGGTCATAG |
| 2075741 | ACGTTGGATGAGACCCAGTGTCTCTCCTGT | ACGTTGGATGAAGATGCCAGTCCGTGGACC |
| 11549918 | ACGTTGGATGTAGATGGTCACGTTCTCCCG | ACGTTGGATGACAGCTAGCGCAGAGCAGGA |

TABLE 14

| dbSNP rs# | Extend Primer | Term Mix |
|---|---|---|
| 1801714 | CCTTCAGCAGGAGCTGGGCCCTC | ACT |
| 2228615 | TAACCTGCGCAGCTGGG | ACT |
| 2569703 | GAGGTGTCACGCTAGTCGAG | ACT |
| 1799969 | CTCCGAGACTGGGAACAGCC | ACG |
| 1059849 | AGGGGCGACTCCACGGA | ACT |
| 3093035 | CTGAGTCCGGATCAGAA | ACG |
| 281439 | GTCCCGCCCCCTCCGTCGCGTGC | ACT |
| 281432 | GGAGTCATGGAGGGTTT | ACT |
| 901886 | TCCGCAGCTCTTTGAAC | ACT |
| 5030382 | CACATTCACGGTCACCT | ACG |
| 2569693 | CCTTCTGCTCGGTGGGTGG | ACT |
| 2569702 | ACCTTGAACCAGATAGAA | ACT |
| 3093030 | AAACATTGTGGGTTGATGG | ACG |
| 2075741 | CCCGGGTACCTCCTTACGCT | ACT |
| 11549918 | CCCCAGGGTGACGTTGCAGA | ACG |

Table 15, below, shows the case and control allele frequencies along with the p-values for the SNPs genotyped. The disease associated allele of column 4 is in bold and the disease associated amino acid of column 5 is also in bold. The chromosome positions provided correspond to NCBI's Build 34.

TABLE 15
Genotyping Results

| | | | | | | | | | Breast |
|---|---|---|---|---|---|---|---|---|---|
| | Position in | | | Amino | | | | | Cancer |
| | SEQ ID | Chromosome | Alleles | Acid | | AF F | | Odds | Associated |
| dbSNP rs# | NO: 1 | Position | (A1/A2) | Change | AF F case | control | p-value | Ratio | Allele |
| 1801714 | 36608 | 10240417 | T/C | L352P | T = 0.010 | T = 0.030 | 0.0734 | 2.26 | C |
| | | | | | C = 0.990 | C = 0.097 | | | |
| 2228615 | 44768 | 10248577 | A/G | T348A | A = 0.340 | A = 0.430 | 0.00236 | 1.47 | G |
| | | | | | G = 0.660 | G = 0.570 | | | |
| 2569703 | 45627 | 10265227 | G/C | | G = 0.508 | G = 0.581 | 0.019 | 1.34 | C |
| | | | | | C = 0.492 | C = 0.419 | | | |
| 1799969 | 36192 | 10255792 | G/A | G241R | G = 0.912 | G = 0.883 | 0.12 | 0.723 | G |
| | | | | | A = 0.088 | A = 0.117 | | | |
| 1059849 | 12054 | 10231654 | A/G | | A = 0.443 | A = 0.364 | 0.00996 | 0.719 | A |
| | | | | | G = 0.557 | G = 0.636 | | | |
| 3093035 | 24373 | 10243973 | G/A | | G = 0.941 | G = 0.951 | 0.466 | 1.22 | A |
| | | | | | A = 0.059 | A = 0.049 | | | |
| 281439 | 41510 | 10261110 | C/G | | C = 0.765 | C = 0.781 | 0.563 | 1.09 | G |
| | | | | | G = 0.235 | G = 0.219 | | | |
| 281432 | 32058 | 10251658 | C/G | | C = 0.549 | C = 0.554 | 0.876 | 1.02 | G |
| | | | | | G = 0.451 | G = 0.446 | | | |
| 901886 | 43531 | 10263131 | T/C | | T = 0.557 | T = 0.483 | 0.0177 | 0.741 | T |
| | | | | | C = 0.443 | C = 0.517 | | | |
| 5030382 | 37083 | 10256683 | G/A | | G = 0.616 | G = 0.536 | 0.0102 | 0.721 | G |
| | | | | | A = 0.384 | A = 0.464 | | | |
| 2569693 | 41304 | 10260904 | T/C | | T = 0.35 | T = 0.451 | 0.0012 | 1.53 | C |
| | | | | | C = 0.65 | C = 0.549 | | | |
| 2569702 | 45347 | 10264947 | T/C | | T = 0.667 | T = 0.562 | 0.000829 | 0.643 | T |
| | | | | | C = 0.333 | C = 0.438 | | | |
| 3093030 | 38803 | 10258403 | C/T | | C = 0.613 | C = 0.541 | 0.0235 | 0.745 | C |
| | | | | | T = 0.387 | T = 0.459 | | | |
| 2075741 | 42498 | 10262098 | C/G | | C = 0.392 | C = 0.466 | 0.0168 | 1.35 | G |
| | | | | | G = 0.608 | G = 0.534 | | | |
| 11549918 | 44338 | 10263938 | G/A | | G = 0.663 | G = 0.558 | 0.000613 | 0.642 | G |
| | | | | | A = 0.337 | A = 0.442 | | | |

### Example 5

### MAPK10 Proximal SNPs

It has been discovered that a polymorphic variation (rs1541998) in a region that encodes MAPK10 is associated with the occurrence of breast cancer (see Examples 1 and 2). Subsequently, SNPs proximal to the incident SNP (rs1541998) were identified and allelotyped in breast cancer sample sets and control sample sets as described in Examples 1 and 2. Approximately ninety-two allelic variants located within the MAPK10 region were identified and allelotyped. The polymorphic variants are set forth in Table 16. The chromosome position provided in column four of Table 16 is based on Genome “Build 34” of NCBI's GenBank.

TABLE 16

| | Position in | | | | | |
|---|---|---|---|---|---|---|
| | SEQ ID | | Chromosome | Allele | Genome | Deduced |
| dbSNP rs# | NO: 2 | Chromosome | Position | Variants | Letter | Iupac |
| 2575681 | 206 | 4 | 87372306 | c/t | g | R |
| 2575680 | 1505 | 4 | 87373605 | a/g | t | Y |
| 2589505 | 3796 | 4 | 87375896 | c/t | a | R |
| 2589504 | 3950 | 4 | 87376050 | g/a | t | Y |
| 2164538 | 4527 | 4 | 87376627 | t/c | a | R |
| 2575679 | 7588 | 4 | 87379688 | a/g | t | Y |
| 10305 | 8482 | 4 | 87380582 | a/t | a | W |
| 2869408 | 9016 | 4 | 87381116 | c/g | g | S |
| 2904086 | 9018 | 4 | 87381118 | c/t | t | Y |
| 934648 | 9747 | 4 | 87381847 | t/c | g | R |
| 2589511 | 12207 | 4 | 87384307 | g/a | g | R |
| 2060589 | 13040 | 4 | 87385140 | g/a | t | Y |
| 2164537 | 13492 | 4 | 87385592 | t/c | g | R |
| 2575678 | 13802 | 4 | 87385902 | a/c | t | K |
| 2575677 | 13918 | 4 | 87386018 | g/c | g | S |
| 2589510 | 14153 | 4 | 87386253 | a/g | c | Y |
| 2589509 | 14370 | 4 | 87386470 | t/g | c | M |
| 2164536 | 15068 | 4 | 87387168 | a/c | t | K |
| 2164535 | 15474 | 4 | 87387574 | t/a | a | W |
| 1946734 | 17117 | 4 | 87389217 | c/g | c | S |
| 2589525 | 17777 | 4 | 87389877 | g/a | g | R |
| 2589523 | 19497 | 4 | 87391597 | c/t | a | R |
| 3755970 | 19646 | 4 | 87391746 | a/c | a | M |
| MAPK10-AA | 21751 | 4 | 87393851 | a/c | g | K |
| 2575675 | 22185 | 4 | 87394285 | g/a | t | Y |
| 1202 | 22703 | 4 | 87394803 | t/c | c | Y |
| 1201 | 22763 | 4 | 87394863 | a/g | a | R |
| 2589516 | 23391 | 4 | 87395491 | g/t | a | M |
| 2575674 | 23841 | 4 | 87395941 | a/t | a | W |
| 2589515 | 23883 | 4 | 87395983 | g/c | g | S |
| 3733367 | 24132 | 4 | 87396232 | c/t | c | Y |
| 958 | 24169 | 4 | 87396269 | c/t | c | Y |
| 2589506 | 25987 | 4 | 87398087 | g/a | t | Y |
| 1436524 | 26072 | 4 | 87398172 | a/g | c | Y |
| 2575672 | 26376 | 4 | 87398476 | c/t | g | R |
| 2589518 | 26614 | 4 | 87398714 | g/a | c | Y |
| 3775164 | 26727 | 4 | 87398827 | t/g | t | K |
| 2589514 | 26827 | 4 | 87398927 | g/a | c | Y |
| 3775166 | 27084 | 4 | 87399184 | t/c | t | Y |
| MAPK10-AB | 30965 | 4 | 87403065 | g/a | c | Y |
| 3775167 | 32436 | 4 | 87404536 | c/t | c | Y |
| 3822035 | 32821 | 4 | 87404921 | t/a | t | W |
| 3775168 | 32979 | 4 | 87405079 | a/t | t | W |
| 3775169 | 33572 | 4 | 87405672 | t/c | t | Y |
| 2043650 | 35142 | 4 | 87407242 | a/g | c | Y |
| 2043649 | 35237 | 4 | 87407337 | t/g | c | M |
| 3775170 | 36014 | 4 | 87408114 | t/a | t | W |
| 1541998 | 36439 | 4 | 87408539 | c/t | a | R |
| MAPK10-AC | 36838 | 4 | 87408938 | g/a | a | R |
| MAPK10-AD | 36889 | 4 | 87408989 | g/a | g | R |
| MAPK10-AE | 38639 | 4 | 87410739 | t/c | t | Y |
| 2282599 | 38657 | 4 | 87410757 | t/g | a | M |
| MAPK10-AF | 38865 | 4 | 87410965 | t/c | t | Y |
| MAPK10-AG | 38885 | 4 | 87410985 | a/g | g | R |
| MAPK10-AH | 38943 | 4 | 87411043 | t/c | t | Y |
| MAPK10-AI | 39035 | 4 | 87411135 | t/c | c | Y |
| MAPK10-AJ | 39046 | 4 | 87411146 | t/c | t | Y |
| 2282598 | 39218 | 4 | 87411318 | c/t | g | R |
| 2282597 | 39241 | 4 | 87411341 | g/a | t | Y |
| 3775171 | 40105 | 4 | 87412205 | a/c | a | M |
| 3775172 | 40240 | 4 | 87412340 | c/t | t | Y |
| 3775173 | 41162 | 4 | 87413262 | t/c | t | Y |
| 3775174 | 42477 | 4 | 87414577 | a/c | a | M |
| 1469870 | 46191 | 4 | 87418291 | g/c | c | S |
| 1436522 | 50467 | 4 | 87422567 | t/c | a | R |
| 1946733 | 52934 | 4 | 87425034 | g/a | c | Y |
| 983362 | 54730 | 4 | 87426830 | t/c | c | Y |
| 3755971 | 58283 | 4 | 87430383 | c/t | t | Y |
| 3822036 | 58378 | 4 | 87430478 | c/g | g | S |
| 3775175 | 59505 | 4 | 87431605 | g/a | a | R |
| 1436525 | 60229 | 4 | 87432329 | g/a | g | R |
| 3822037 | 61108 | 4 | 87433208 | c/g | c | S |
| 3775176 | 62587 | 4 | 87434687 | g/a | a | R |
| 993593 | 63133 | 4 | 87435233 | c/t | c | Y |
| 1436527 | 63616 | 4 | 87435716 | c/t | c | Y |
| 1436529 | 65377 | 4 | 87437477 | t/c | c | Y |
| 3775180 | 65442 | 4 | 87437542 | a/c | a | M |
| 3775181 | 65548 | 4 | 87437648 | t/g | g | K |
| 3775182 | 65878 | 4 | 87437978 | t/g | g | K |
| 3775183 | 66222 | 4 | 87438322 | g/a | a | R |
| 3775184 | 66354 | 4 | 87438454 | a/g | a | R |
| 733245 | 67224 | 4 | 87439324 | a/t | a | W |
| 3775185 | 68198 | 4 | 87440298 | t/g | g | K |
| 1561154 | 68729 | 4 | 87440829 | t/c | c | Y |
| 3775186 | 69058 | 4 | 87441158 | a/c | c | M |
| 3775187 | 69527 | 4 | 87441627 | t/c | t | Y |
| 1010778 | 70774 | 4 | 87442874 | a/g | t | Y |
| 2282596 | 71232 | 4 | 87443332 | t/a | t | W |
| 2282595 | 71943 | 4 | 87444043 | a/c | t | K |
| 2118044 | 73397 | 4 | 87445497 | a/t | a | W |
| 1469869 | 76322 | 4 | 87448422 | c/t | c | Y |
| MAPK10-AK | 110704 | 4 | 87482804 | a/g | t | Y |

### Assay for Verifying and Allelotyping SNPs

The methods used to verify and allelotype the proximal SNPs of Table 16 are the same methods described in Examples 1 and 2 herein. The PCR primers and extend primers used in these assays are provided in Table 17 (SEQ ID NOS 679-862, respectively, in order of appearance) and Table 18 (SEQ ID NOS 863-954, respectively, in order of appearance), respectively.

TABLE 17

| | | |
|---|---|---|
| dbSNP rs# | Forward PCR primer | Reverse PCR primer |

| | | |
|---|---|---|
| 2575681 | ACGTTGGATGTTATATAGCCTTCTTTTCTC | ACGTTGGATGTTCACTGCTAACATGCATGG |
| 2575680 | ACGTTGGATGGAGCCCAATACAATCAGGTG | ACGTTGGATGGCCCTGAAGTTTTTGAATGG |
| 2589505 | ACGTTGGATGCATTTTATGAGAAGATGCAC | ACGTTGGATGAGACTGTAGCCTAAATGAGG |
| 2589504 | ACGTTGGATGCTGTGTGATTTGGACAACCC | ACGTTGGATGGGATAGGAAACATATTAAGG |
| 2164538 | ACGTTGGATGAGAGGTCATCTTAATGGGCC | ACGTTGGATGTACTGCAGAGCTCTCCCTTG |
| 2575679 | ACGTTGGATGTAAGCCAGTAACACATGCCG | ACGTTGGATGCTTTCCTGCTGCATTTAGTG |
| 10305 | ACGTTGGATGGGTGAAGCTTGAAAGCAAGC | ACGTTGGATGTTCAAGAATTATTTTATTGCAAGTC |
| 2869408 | ACGTTGGATGGCTTTGAATTACTCTGTCCC | ACGTTGGATGTCCCAGTACCTAAGTAGCAG |
| 2904086 | ACGTTGGATGGCTTTGAATTACTCTGTCCC | ACGTTGGATGTCCCAGTACCTAAGTAGCAG |
| 934648 | ACGTTGGATGTGTACTGCTTTCATCCTTGC | ACGTTGGATGACTGGTTGATACCATAGGAC |
| 2589511 | ACGTTGGATGGTAGAGCTGACATCATAGGC | ACGTTGGATGCCTACTGTGTTAGCCTCACT |
| 2060589 | ACGTTGGATGACAACTACGTGTAACTGTCC | ACGTTGGATGCCAATACCTTTAACACCCAAC |
| 2164537 | ACGTTGGATGAACACACTTAGTACCCACGC | ACGTTGGATGCAAGGCAAAATGTTTCCAGC |
| 2575678 | ACGTTGGATGAAGCACCATTTGTGGCTCAG | ACGTTGGATGCTTCAAGAGGCCATACAGAC |
| 2575677 | ACGTTGGATGGAAGGATAAGCCACAGTGAG | ACGTTGGATGGATGCCAATTTGGTTTGCCC |
| 2589510 | ACGTTGGATGAATGGCATGGGAACTTGGAC | ACGTTGGATGTGCACCACACTGAAGTGATC |
| 2589509 | ACGTTGGATGATACGCAGGTTGTAGAGAGG | ACGTTGGATGTGAATCATGGTTGCCTCCTG |
| 2164536 | ACGTTGGATGCTGGAGAACAAAAGACCACC | ACGTTGGATGGTGATGAAAACCATGTGAGC |
| 2164535 | ACGTTGGATGGAATGATGTAAACGTTGGAG | ACGTTGGATGACCAGCACTATTACCCATGC |
| 1946734 | ACGTTGGATGGTCAGCAAATCTGCCTTCAC | ACGTTGGATGAACCTGCTTTGTGGTCTTCG |
| 2589525 | ACGTTGGATGGTATTTAAATTAGTGGTGTG | ACGTTGGATGAAGAACATTGAAAGAAGCAG |
| 2589523 | ACGTTGGATGAGCAGGGTTAAATTTCCCAG | ACGTTGGATGTCATGTAGCTAAACAAAGGC |
| 3755970 | ACGTTGGATGGTGACCATGTAGAAATCTGTG | ACGTTGGATGTACAACTAGTATCTACAGAC |
| MAPK10-AA | ACGTTGGATGCTTGGGAAATAACAGGTGAC | ACGTTGGATGCACTTGTCTGTCTTAACACAC |
| 2575675 | ACGTTGGATGTTGGGAAGGTACTAACAGCG | ACGTTGGATGTCGTACCTGCATAAGTGGTG |
| 1202 | ACGTTGGATGTCAAGCAATAGAGACCACAG | ACGTTGGATGTAATCTCAGAATGGCAGCAC |
| 1201 | ACGTTGGATGCCTGTGGTCTCTATTGCTTG | ACGTTGGATGTGCCAGTGCTCTGAAAACTG |
| 2589516 | ACGTTGGATGACTCACACTGTGGTTTGGGG | ACGTTGGATGCATACTCTGCCAAAGTTTTA |
| 2575674 | ACGTTGGATGCTCTCCTACTCTTTACTGTC | ACGTTGGATGGTGGGTAACAGTTTTCAGGC |
| 2589515 | ACGTTGGATGGCCAAACCATTTTGTGCCTG | ACGTTGGATGCACCTGTATACCAATTTGTAG |
| 3733367 | ACGTTGGATGCAGATGGGTTTATGTCAGAG | ACGTTGGATGGGCTCTACCAAGACATAATG |
| 958 | ACGTTGGATGCCCAGTGCATTATGTCTTGG | ACGTTGGATGATCCGCATGTGTCTGTATTC |
| 2589506 | ACGTTGGATGTGTTATGCGGGAGTATAAGG | ACGTTGGATGGCAACTCAGCTAGCCTTTAC |
| 1436524 | ACGTTGGATGATTGTTTCCAGGGTGCTCTG | ACGTTGGATGGAGTTGCCAGTAGCTTTGAG |
| 2575672 | ACGTTGGATGCTCCAGGAGCAAGGATTATG | ACGTTGGATGATAGTGTTATCACATAGACC |
| 2589518 | ACGTTGGATGAATGATATGCACCGATCTTC | ACGTTGGATGCCAGGCAAAAAGAATGACCG |
| 3775164 | ACGTTGGATGAAGTTTCCCTGGTCGTGATC | ACGTTGGATGGAACATGAAAAATTCATAAGC |
| 2589514 | ACGTTGGATGCTTAAATGTCTCTAGAAAAGG | ACGTTGGATGTATACATTGTCCTGATAGAG |
| 3775166 | ACGTTGGATGCTGAGGAGTCCATCATAGTG | ACGTTGGATGCTGTTTTTCACCCCCGATTC |
| MAPK10-AB | ACGTTGGATGGATAAGTATTGGCTTAATCTG | ACGTTGGATGTTTCATTGCTCATGGATTAG |
| 3775167 | ACGTTGGATGGCTTCTGATTTTATATGGCAC | ACGTTGGATGGAAACAAGCAGATGTCATGG |
| 3822035 | ACGTTGGATGATAAATGCCTCCTTGCCTGC | ACGTTGGATGGAACTCCTGTTTATTGCCTAC |
| 3775168 | ACGTTGGATGTCTCACATTGACTGGACAAC | ACGTTGGATGCCAAGCAGTTTGCGAAAATC |
| 3775169 | ACGTTGGATGATGCTGAACAACAGGATGGG | ACGTTGGATGGGGAGAGAATGGTTGCATAT |
| 2043650 | ACGTTGGATGCTGTGCCTTGCACATAGTAG | ACGTTGGATGGCTGAGGGAGAAATTGAGTG |
| 2043649 | ACGTTGGATGGTCCTACTAGTCCCTGTATG | ACGTTGGATGGCACTACATGGGACACAAAG |
| 3775170 | ACGTTGGATGCCCATTTTTGCTAGCAGGAG | ACGTTGGATGCCTAAGACCTATGCTCTCAC |
| 1541998 | ACGTTGGATGCTGATTATTCTGATGGTAATG | ACGTTGGATGGCCCATGTTAACATTTTCTTC |
| MAPK10-AC | ACGTTGGATGCTCTCTGCTAATTACAAGGC | ACGTTGGATGTACGTTTTGATGGGGTTGAG |
| MAPK10-AD | ACGTTGGATGAGATACCTATTTGTGCCAGG | ACGTTGGATGTACGTTTTGATGGGGTTGAG |
| MAPK10-AE | ACGTTGGATGTGTCTTTTCATGCCGAAAGC | ACGTTGGATGCGACAAACACTGACAATCAG |
| 2282599 | ACGTTGGATGGACTGCTATTCTCTTAGACC | ACGTTGGATGCATTCTTGAGCAAATGTGAC |
| MAPK10-AF | ACGTTGGATGGTCATATATCTGAGGTGGAG | ACGTTGGATGCAAGGTATTCTCGTGCATTG |
| MAPK10-AG | ACGTTGGATGGTCATATATCTGAGGTGGAG | ACGTTGGATGCAAGGTATTCTCGTGCATTG |
| MAPK10-AH | ACGTTGGATGCGAAATAAATGAGGGCAAGG | ACGTTGGATGGGAGGCTGCCGAATAAAAAC |
| MAPK10-AI | ACGTTGGATGTGGGTCAGCAGCAAATATTG | ACGTTGGATGTGCCCTCATTTATTTCGGTG |
| MAPK10-AJ | ACGTTGGATGTGGGTCAGCAGCAAATATTG | ACGTTGGATGTGCCCTCATTTATTTCGGTG |
| 2282598 | ACGTTGGATGGTGAATGAAGGAAAAGTAGC | ACGTTGGATGCACGCCTAAGCAATTAATGAC |
| 2282597 | ACGTTGGATGACACTTGATTACAATGGCCC | ACGTTGGATGGCATGGTTCTGTTATAAGGC |
| 3775171 | ACGTTGGATGCTGTCATGATTCAAGCTACC | ACGTTGGATGTTTGGCTCATGAACAGCACC |
| 3775172 | ACGTTGGATGCTGCAAAGTATCTGGCTATG | ACGTTGGATGGCTCCGTGAGTAGTTATTTC |
| 3775173 | ACGTTGGATGTAAATTTGCAGAGGCCGTCG | ACGTTGGATGCAAGAGGGCTGCTTTAAACC |
| 3775174 | ACGTTGGATGCATCCTTGATCCACCCTTTG | ACGTTGGATGCATGTCGTCAGAAGTGACAA |
| 1469870 | ACGTTGGATGATGGCACAGTTTAGCATGTC | ACGTTGGATGATACTGAGCTCCATTTTGGG |
| 1436522 | ACGTTGGATGATTGGAAGGAGGAAGCATAG | ACGTTGGATGGGAATTGAAATTGGCATTGC |
| 1946733 | ACGTTGGATGTAGCTTCTAAACATCTCTTG | ACGTTGGATGGCAGGAGGATAGATCTGTAG |
| 983362 | ACGTTGGATGCACATTGCTCCCTCCTTTAC | ACGTTGGATGGCGGCCGAAGAGTACTATTT |
| 3755971 | ACGTTGGATGGTGGTAACAGATTGCTGTAC | ACGTTGGATGGCGTAAAGAACTTTTGGTGAC |
| 3822036 | ACGTTGGATGCAACACATACCTTAGGTAAGA | ACGTTGGATGCTGAATTCCTTCGATGTTCC |
| 3775175 | ACGTTGGATGCATTATAAAAACAGTTTCTTG | ACGTTGGATGGAGTAGACAGTGGCTATTAA |
| 1436525 | ACGTTGGATGGCTTACACTAGCTACTTGGG | ACGTTGGATGGTGCAATCTTGGTTCACTGC |
| 3822037 | ACGTTGGATGTCCCACCCTGACTTCTTTGC | ACGTTGGATGGTAATCCATAAACTGTGGGAG |
| 3775176 | ACGTTGGATGTAGTCCAAGTATTTCCCAAG | ACGTTGGATGAAAAGGTCACCAGTGACCTG |
| 993593 | ACGTTGGATGTAGGCACTGCAGATCTTTTC | ACGTTGGATGCACTAAAATAATAAGAAAGCTC |
| 1436527 | ACGTTGGATGCTGTATAGAGAGCTGTTTGC | ACGTTGGATGAGCACTGTGAGTTAAACCTG |
| 1436529 | ACGTTGGATGAATGTTGGACCACATGTACG | ACGTTGGATGCTATGGCAGCAGAAGAGTAG |
| 3775180 | ACGTTGGATGTCCACTGAATGTGCACTTCC | ACGTTGGATGCAAGACTGTAGCCCTACAAC |
| 3775181 | ACGTTGGATGCTTCTGCCAAGACTATCAGG | ACGTTGGATGGTAAGCATCCCCCTAATAAT |
| 3775182 | ACGTTGGATGGCTGGACTCTATTAGGCCAT | ACGTTGGATGGATATCTCCCTCCTATTGGC |
| 3775183 | ACGTTGGATGTGCAGATATGTAGGCCAAGC | ACGTTGGATGGATCTCTGATCTTAGACCAC |
| 3775184 | ACGTTGGATGGTTCTACTTTGACCACAGGC | ACGTTGGATGGACCAGCAACCATGATGAAG |
| 733245 | ACGTTGGATGTAGTCTAGTGGAGGATGTCG | ACGTTGGATGCCAGCTATAATGTAGACCAC |
| 3775185 | ACGTTGGATGTGTGGAATCTATAATGTGTC | ACGTTGGATGACCTACCTCTTCTTTGCCAG |
| 1561154 | ACGTTGGATGAATAGAAGATGCTCTAACTC | ACGTTGGATGGAAGATGCAAATAAGTCAACC |
| 3775186 | ACGTTGGATGGATATGCTATGTGTGCTAAG | ACGTTGGATGTTGTACTTTGGTAGTTTGGG |
| 3775187 | ACGTTGGATGAATCATGATCCCAGGGCAAG | ACGTTGGATGTAGCACCTTCAGGATCTTTC |
| 1010778 | ACGTTGGATGGGATTTGTTCTTAATCTTTC | ACGTTGGATGCAGAGGAAAGAAAACTGAAAG |
| 2282596 | ACGTTGGATGGGTGGCTTTGTGAAACCTTG | ACGTTGGATGTCATACTGATCAACCTGAAG |
| 2282595 | ACGTTGGATGAAGGAAATTTGTCAGAGAGG | ACGTTGGATGCTTTTCCATCACATCAAGGG |
| 2118044 | ACGTTGGATGGTCATTGCCTCTAGCTAGTG | ACGTTGGATGAACAACTTGGCTAATTCTAC |
| 1469869 | ACGTTGGATGTTCGATATATCAGAGCCTTG | ACGTTGGATGCATGGCGAGGAAATCTGTTT |
| MAPK10-AK | ACGTTGGATGTTTCTCTCGTTTCCTCTGTC | ACGTTGGATGACTTACACATCTTGGAACTC |

TABLE 18

| dbSNP rs# | Extend Primer | Term Mix |
|---|---|---|
| 2575681 | TTCTTTTCTCTTTTAGGAATCT | ACG |
| 2575680 | GGATGCATGGTTTCTCTAAT | ACT |
| 2589505 | GTTTTAGCATAATTGCTTCTTTA | ACG |
| 2589504 | GTGCTAGGATCCTCAGT | ACG |
| 2164538 | GTCACATTCTTACCCTC | ACT |
| 2575679 | CTTCCTGGACATTAAATTGT | ACT |
| 10305 | AGCTAAATTGCAACAACA | ACG |
| 2869408 | CGAATCTCTTTAACTGCTG | ACT |
| 2904086 | GAATCTCTTTAACTGCTGGA | ACG |
| 934648 | ACTCTCCCACTGAGCAAGC | ACT |
| 2589511 | CTCCAAAGGATACCCAGA | ACG |
| 2060589 | ACTCTATCCATGTCTACAC | ACG |
| 2164537 | CGGCTTCTACTCTCTTATTCA | ACT |
| 2575678 | TGTGGCTCAGGTCCAGG | ACT |
| 2575677 | GGAATGAGGGCAACAGGA | ACT |
| 2589510 | TTTGGCAACAGGTAACCAGC | ACT |
| 2589509 | AGGGCTGCAGGGAAGAT | ACT |
| 2164536 | CCTGTGTTCCTTTGTATTTATAT | ACT |
| 2164535 | GATAAATGTGAGATTGAGAGA | CGT |
| 1946734 | GCATTCTAGTGGAGAAGTCA | ACT |
| 2589525 | TTAGTGGTGTGACTTGCA | ACG |
| 2589523 | TTTCCCAGATTAATTATCAGATT | ACG |
| 3755970 | GGTTTCTTCTAAAACTGACCT | ACT |
| MAPK10-AA | GGTGACTATTTAAGAAATATTTGG | ACT |
| 2575675 | GTTCTTGCCTGGTTTAC | ACG |
| 1202 | CCACCTGCACCATCGCCAT | ACT |
| 1201 | TCTATTGCTTGAAGAGAGAAAG | ACT |
| 2589516 | GGTTTGGGGGTTTCATT | CGT |
| 2575674 | TATTCACACCTGCCTTC | CGT |
| 2589515 | GAAAACTGTTACCCACTC | ACT |
| 3733367 | GTAATAGATCACATGAAATGGAC | ACG |
| 958 | TTATGTCTTGGTAGAGCC | ACG |
| 2589506 | GAGAAGAAACCTGCCCA | ACG |
| 1436524 | CCAGGGTGCTCTGGTTTAATT | ACT |
| 2575672 | CAAGGATTATGTTAACCACT | ACG |
| 2589518 | TGCACCGATCTTCAAATAAA | ACG |
| 3775164 | TTTTTTGGGATCTTGATATTTTTA | ACT |
| 2589514 | AGAAAAGGTTTTTAAAGTCCTC | ACG |
| 3775166 | AACTTATGAAAGAATATGAAGGAT | ACT |
| MAPK10-AB | ATTGGCTTAATCTGTACATCAATT | ACG |
| 3775167 | TAAGAGAAGTCTTCAGTGCTT | ACG |
| 3822035 | CTGGATGATCCATTCAAG | CGT |
| 3775168 | ACTGGACAACTGAACAC | CGT |
| 3775169 | GCAGAGATTTTTCAAAATCTCTAA | ACT |
| 2043650 | GCACATAGTAGTAGCTCA | ACT |
| 2043649 | CCTCTTGTCTTATTATCCC | ACT |
| 3775170 | TTTTTAAAGCTGAAAATAAACCA | CGT |
| 1541998 | ATTATTCTGATGGTAATGATCCAG | ACG |
| MAPK10-AC | TAGAGCAGTAAAGGAATCTCAA | ACG |
| MAPK10-AD | TATTTGTGCCAGGCTCTCTG | ACG |
| MAPK10-AE | CATGCCGAAAGCAATGTCAC | ACT |
| 2282599 | CTAAAACTTCATCTGTCTTTTCA | ACT |
| MAPK10-AF | TCTGAGGTGGAGGCTGCC | ACT |
| MAPK10-AG | TCTGAGGTGGAGGCTGCC | ACT |
| MAPK10-AH | GGGCAAGGTATTCTCGTGC | ACT |
| MAPK10-AI | AATGAATCCTCCATAAGTTTACA | ACT |
| MAPK10-AJ | TTGCAATATGAATGAATCCTCC | ACT |
| 2282598 | AGGAAAAGTAGCTTCTGGG | ACG |
| 2282597 | CAGAAGCTACTTTTCCTTCA | ACG |
| 3775171 | GAATTAGAGAAGGGTCACT | ACT |
| 3775172 | TATGAAAATAGAAGACTTTGCC | ACG |
| 3775173 | GCCGTCGAACAAATACT | ACT |
| 3775174 | GCTTCTCTAAGTACAGCTC | ACT |
| 1469870 | CTTATATTCTCTGTGGCACCAA | ACT |
| 1436522 | GAGGAAGCATAGATTTGGTGT | ACT |
| 1946733 | CTAAACATCTCTTGAATATTCTG | ACG |
| 983362 | GCTCAAATGTTACCTTCTCAAA | ACT |
| 3755971 | AACAGCATTCTGGCATATA | ACG |
| 3822036 | CAGAAAAAGTGCTTGAGG | ACT |
| 3775175 | CAGTTTCTTGTGGTCCC | ACG |
| 1436525 | GGCTTAAACCTGGGAGG | ACG |
| 3822037 | TTTGCTTATTTCATAGAAGGAAT | ACT |
| 3775176 | TATTTCCCAAGTGCCCA | ACG |
| 993593 | AGCAACAGAGGCTTTTTCTA | ACG |
| 1436527 | GAGCTGTTTGCATTTATAACTCA | ACG |
| 1436529 | ACCACATGTACGTAAGGGGA | ACT |
| 3775180 | TATGTGTTGATGTCACTCTT | ACT |
| 3775181 | GGTTGGTATAGTACTTGCGAT | ACT |
| 3775182 | CTGTCAGTTGCCTTAGG | ACT |
| 3775183 | AGTCAAGACCAGCTGGG | ACG |
| 3775184 | CTCTTTCTTCTGATCCC | ACT |
| 733245 | CCTTAGATTCCCAAACAAAAC | CGT |
| 3775185 | TTTGGTTTAAACTAATGACACTA | ACT |
| 1561154 | AGATGCTCTAACTCTGGTTCA | ACT |
| 3775186 | GCTAAGCTATTAGTTATACTACGA | ACT |
| 3775187 | AGTGCATTACAGTGGTC | ACT |
| 1010778 | TTGAAATACTGTTTGTTTCCCCAA | ACT |
| 2282596 | GAAACCTTGCATGAACT | CGT |
| 2282595 | AAGTATGGAATAGTATCTTCCT | ACT |
| 2118044 | GTGGGGTTAGATATTATTTCCTGA | CGT |
| 1469869 | AAACACCATCTACTCTGAAGAA | ACG |
| MAPK10-AK | CGTTTCCTCTGTCCCTTCC | ACT |

### Genetic Analysis of Allelotyping Results

Allelotyping results are shown for cases and controls in Table 19. The allele frequency for the A2 allele is noted in the fifth and sixth columns for breast cancer pools and control pools, respectively, where “AF” is allele frequency. SNPs with blank allele frequencies were untyped.

TABLE 19

| | Position in | | | | | | | Breast Cancer |
|---|---|---|---|---|---|---|---|---|
| | SEQ ID | Chromosome | A1/A2 | | | | | Associated |
| dbSNP rs# | NO: 2 | Position | Allele | Case AF | Control AF | p-Value | OR | Allele |
| 2575681 | 206 | 87372306 | C/T | C = 0.610 | C = 0.631 | 0.499 | 1.09 | T |
| | | | | T = 0.390 | T = 0.369 | | | |
| 2575680 | 1505 | 87373605 | A/G | A = 0.404 | A = 0.411 | 0.84 | 1.03 | G |
| | | | | G = 0.596 | G = 0.589 | | | |
| 2589505 | 3796 | 87375896 | C/T | C = 0.515 | C = 0.500 | 0.655 | 0.94 | C |
| | | | | T = 0.485 | T = 0.500 | | | |
| 2589504 | 3950 | 87376050 | G/A | G = 0.744 | G = 0.725 | 0.506 | 0.91 | G |
| | | | | A = 0.256 | A = 0.275 | | | |
| 2164538 | 4527 | 87376627 | T/C | T = 0.595 | T = 0.586 | 0.775 | 0.96 | T |
| | | | | C = 0.405 | C = 0.414 | | | |
| 2575679 | 7588 | 87379688 | A/G | A = 0.968 | A = 0.988 | 0.185 | 2.68 | G |
| | | | | G = 0.032 | G = 0.012 | | | |
| 10305 | 8482 | 87380582 | A/T | A = 0.303 | A = | | | |
| | | | | T = 0.697 | T = | | | |
| 2869408 | 9016 | 87381116 | C/G | C = 0.292 | C = 0.287 | 0.879 | 0.98 | C |
| | | | | G = 0.708 | G = 0.713 | | | |
| 2904086 | 9018 | 87381118 | C/T | C = 0.003 | C = 0.002 | 0.822 | 0.40 | C |
| | | | | T = 0.997 | T = 0.998 | | | |
| 934648 | 9747 | 87381847 | T/C | T = 0.345 | T = 0.337 | 0.786 | 0.96 | T |
| | | | | C = 0.655 | C = 0.663 | | | |
| 2589511 | 12207 | 87384307 | G/A | G = 0.599 | G = | | | |
| | | | | A = 0.401 | A = | | | |
| 2060589 | 13040 | 87385140 | G/A | G = 0.001 | G = 0.008 | 0.431 | 13.10 | A |
| | | | | A = 0.999 | A = 0.992 | | | |
| 2164537 | 13492 | 87385592 | T/C | T = 0.737 | T = 0.695 | 0.197 | 0.81 | T |
| | | | | C = 0.263 | C = 0.305 | | | |
| 2575678 | 13802 | 87385902 | A/C | A = 0.886 | A = 0.928 | 0.181 | 1.66 | C |
| | | | | C = 0.114 | C = 0.072 | | | |
| 2575677 | 13918 | 87386018 | G/C | G = 0.080 | G = 0.027 | 0.00252 | 0.32 | G |
| | | | | C = 0.920 | C = 0.973 | | | |
| 2589510 | 14153 | 87386253 | A/G | A = 0.592 | A = 0.582 | 0.767 | 0.96 | A |
| | | | | G = 0.408 | G = 0.418 | | | |
| 2589509 | 14370 | 87386470 | T/G | T = 0.799 | T = 0.785 | 0.599 | 0.92 | T |
| | | | | G = 0.201 | G = 0.215 | | | |
| 2164536 | 15068 | 87387168 | A/C | A = 0.378 | A = 0.398 | 0.509 | 1.09 | C |
| | | | | C = 0.622 | C = 0.602 | | | |
| 2164535 | 15474 | 87387574 | T/A | T = 0.430 | T = 0.426 | 0.891 | 0.98 | T |
| | | | | A = 0.570 | A = 0.574 | | | |
| 1946734 | 17117 | 87389217 | C/G | C = 0.003 | C = 0.001 | 0.782 | 0.22 | C |
| | | | | G = 0.997 | G = 0.999 | | | |
| 2589525 | 17777 | 87389877 | G/A | G = 0.607 | G = 0.606 | 0.983 | 1.00 | G |
| | | | | A = 0.393 | A = 0.394 | | | |
| 2589523 | 19497 | 87391597 | C/T | C = 0.225 | C = 0.186 | 0.139 | 0.79 | C |
| | | | | T = 0.775 | T = 0.814 | | | |
| 3755970 | 19646 | 87391746 | A/C | A = 0.880 | A = 0.894 | 0.525 | 1.15 | C |
| | | | | C = 0.120 | C = 0.106 | | | |
| MAPK10-AA | 21751 | 87393851 | A/C | A = 0.014 | A = | | | |
| | | | | C = 0.986 | C = | | | |
| 2575675 | 22185 | 87394285 | G/A | G = 0.347 | G = 0.305 | 0.154 | 0.83 | G |
| | | | | A = 0.653 | A = 0.695 | | | |
| 1202 | 22703 | 87394803 | T/C | T = 0.238 | T = 0.241 | 0.909 | 1.02 | C |
| | | | | C = 0.762 | C = 0.759 | | | |
| 1201 | 22763 | 87394863 | A/G | A = 0.872 | A = 0.883 | 0.611 | 1.11 | G |
| | | | | G = 0.128 | G = 0.117 | | | |
| 2589516 | 23391 | 87395491 | G/T | G = 0.575 | G = 0.519 | 0.0708 | 0.80 | G |
| | | | | T = 0.425 | T = 0.481 | | | |
| 2575674 | 23841 | 87395941 | A/T | A = 0.419 | A = 0.340 | 0.0107 | 0.71 | A |
| | | | | T = 0.581 | T = 0.660 | | | |
| 2589515 | 23883 | 87395983 | G/C | G = 0.586 | G = 0.544 | 0.211 | 0.84 | G |
| | | | | C = 0.414 | C = 0.456 | | | |
| 3733367 | 24132 | 87396232 | C/T | C = 1.000 | C = 1.000 | 0.957 | 0.54 | C |
| | | | | T = 0.000 | T = 0.000 | | | |
| 958 | 24169 | 87396269 | C/T | C = 0.833 | C = 0.842 | 0.69 | 1.07 | T |
| | | | | T = 0.167 | T = 0.158 | | | |
| 2589506 | 25987 | 87398087 | G/A | G = 0.568 | G = 0.51 | 0.0657 | 0.79 | G |
| | | | | A = 0.432 | A = 0.490 | | | |
| 1436524 | 26072 | 87398172 | A/G | A = 0.341 | A = 0.248 | 0.00126 | 0.64 | A |
| | | | | G = 0.659 | G = 0.752 | | | |
| 2575672 | 26376 | 87398476 | C/T | C = 0.726 | C = 0.814 | 0.00125 | 1.65 | T |
| | | | | T = 0.274 | T = 0.186 | | | |
| 2589518 | 26614 | 87398714 | G/A | G = 0.806 | G = 0.868 | 0.00984 | 1.58 | A |
| | | | | A = 0.194 | A = 0.132 | | | |
| 3775164 | 26727 | 87398827 | T/G | T = 0.934 | T = 0.921 | 0.523 | 0.83 | T |
| | | | | G = 0.066 | G = 0.079 | | | |
| 2589514 | 26827 | 87398927 | G/A | G = 0.556 | G = 0.639 | 0.0199 | 1.41 | A |
| | | | | A = 0.444 | A = 0.361 | | | |
| 3775166 | 27084 | 87399184 | T/C | T = 0.751 | T = 0.833 | 0.00351 | 1.65 | C |
| | | | | C = 0.249 | C = 0.167 | | | |
| MAPK10-AB | 30965 | 87403065 | G/A | G = 0.814 | G = 0.861 | 0.0453 | 1.42 | A |
| | | | | A = 0.186 | A = 0.139 | | | |
| 3775167 | 32436 | 87404536 | C/T | C = 0.845 | C = 0.850 | 0.858 | 1.04 | T |
| | | | | T = 0.155 | T = 0.150 | | | |
| 3822035 | 32821 | 87404921 | T/A | T = 0.998 | T = 1.000 | 0.761 | | A |
| | | | | A = 0.002 | A = 0 | | | |
| 3775168 | 32979 | 87405079 | A/T | A = 0.380 | A = 0.261 | 0.00252 | 0.57 | A |
| | | | | T = 0.620 | T = 0.739 | | | |
| 3775169 | 33572 | 87405672 | T/C | T = 0.829 | T = 0.870 | 0.106 | 1.38 | C |
| | | | | C = 0.171 | C = 0.130 | | | |
| 2043650 | 35142 | 87407242 | A/G | A = 0.308 | A = 0.215 | 0.00102 | 0.62 | A |
| | | | | G = 0.692 | G = 0.785 | | | |
| 2043649 | 35237 | 87407337 | T/G | T = 0.302 | T = 0.241 | 0.0305 | 0.73 | T |
| | | | | G = 0.698 | G = 0.759 | | | |
| 3775170 | 36014 | 87408114 | T/A | T = 0.791 | T = 0.784 | 0.782 | 0.96 | T |
| | | | | A = 0.209 | A = 0.216 | | | |
| 1541998 | 36439 | 87408539 | C/T | C = 0.283 | C = 0.228 | 0.0604 | 0.75 | C |
| | | | | T = 0.717 | T = 0.772 | | | |
| MAPK10-AC | 36838 | 87408938 | G/A | G = 0.034 | G = 0.013 | 0.176 | 0.36 | G |
| | | | | A = 0.966 | A = 0.987 | | | |
| MAPK10-AD | 36889 | 87408989 | G/A | G = 0.923 | G = 0.958 | 0.081 | 1.90 | A |
| | | | | A = 0.077 | A = 0.042 | | | |
| MAPK10-AE | 38639 | 87410739 | T/C | T = 0.919 | T = 0.931 | 0.491 | 1.20 | C |
| | | | | C = 0.081 | C = 0.069 | | | |
| 2282599 | 38657 | 87410757 | T/G | T = 1.000 | T = 1.000 | 0.976 | 0.64 | T |
| | | | | G = 0.000 | G = 0.000 | | | |
| MAPK10-AF | 38865 | 87410965 | T/C | T = 0.300 | T = 0.222 | 0.00536 | 0.66 | T |
| | | | | C = 0.700 | C = 0.778 | | | |
| MAPK10-AG | 38885 | 87410985 | A/G | A = 0.249 | A = 0.192 | 0.0626 | 0.71 | A |
| | | | | G = 0.751 | G = 0.808 | | | |
| MAPK10-AH | 38943 | 87411043 | T/C | T = 0.945 | T = 0.953 | 0.649 | 1.17 | C |
| | | | | C = 0.055 | C = 0.047 | | | |
| MAPK10-AI | 39035 | 87411135 | T/C | T = 0.128 | T = 0.058 | 0.000413 | 0.41 | T |
| | | | | C = 0.872 | C = 0.942 | | | |
| MAPK10-AJ | 39046 | 87411146 | T/C | T = 0.823 | T = 0.877 | 0.0241 | 1.53 | C |
| | | | | C = 0.177 | C = 0.123 | | | |
| 2282598 | 39218 | 87411318 | C/T | C = 0.971 | C = 0.966 | 0.735 | 0.86 | C |
| | | | | T = 0.029 | T = 0.034 | | | |
| 2282597 | 39241 | 87411341 | G/A | G = 0.184 | G = 0.197 | 0.613 | 1.09 | A |
| | | | | A = 0.816 | A = 0.803 | | | |
| 3775171 | 40105 | 87412205 | A/C | A = 1.000 | A = 1.000 | 0.998 | 0.94 | A |
| | | | | C = 0.000 | C = 0.000 | | | |
| 3775172 | 40240 | 87412340 | C/T | C = 0.003 | C = 0.003 | 0.995 | 0.98 | C |
| | | | | T = 0.997 | T = 0.997 | | | |
| 3775173 | 41162 | 87413262 | T/C | T = 0.841 | T = 0.852 | 0.652 | 1.09 | C |
| | | | | C = 0.159 | C = 0.148 | | | |
| 3775174 | 42477 | 87414577 | A/C | A = 1.000 | A = 0.999 | 0.947 | 0.55 | A |
| | | | | C = 0.000 | C = 0.001 | | | |
| 1469870 | 46191 | 87418291 | G/C | G = 0.884 | G = 0.934 | 0.013 | 1.85 | C |
| | | | | C = 0.116 | C = 0.066 | | | |
| 1436522 | 50467 | 87422567 | T/C | T = 0.84 | T = 0.876 | 0.122 | 1.34 | C |
| | | | | C = 0.160 | C = 0.124 | | | |
| 1946733 | 52934 | 87425034 | G/A | G = 0.763 | G = 0.773 | 0.709 | 1.06 | A |
| | | | | A = 0.237 | A = 0.227 | | | |
| 983362 | 54730 | 87426830 | T/C | T = 0.002 | T = 0.003 | 0.934 | 1.40 | C |
| | | | | C = 0.998 | C = 0.997 | | | |
| 3755971 | 58283 | 87430383 | C/T | C = 0.001 | C = 0.002 | 0.878 | 3.66 | T |
| | | | | T = 0.999 | T = 0.998 | | | |
| 3822036 | 58378 | 87430478 | C/G | C = 0 | C = 0.001 | 0.944 | | G |
| | | | | G = 1.000 | G = 0.999 | | | |
| 3775175 | 59505 | 87431605 | G/A | G = 0.001 | G = 0.005 | 0.67 | 4.64 | A |
| | | | | A = 0.999 | A = 0.995 | | | |
| 1436525 | 60229 | 87432329 | G/A | G = 0.945 | G = 0.961 | 0.297 | 1.43 | A |
| | | | | A = 0.055 | A = 0.039 | | | |
| 3822037 | 61108 | 87433208 | C/G | C = 0.054 | C = 0.085 | 0.102 | 1.61 | G |
| | | | | G = 0.946 | G = 0.915 | | | |
| 3775176 | 62587 | 87434687 | G/A | G = 0.034 | G = 0.088 | 0.0283 | 2.78 | A |
| | | | | A = 0.966 | A = 0.912 | | | |
| 993593 | 63133 | 87435233 | C/T | C = 0.998 | C = 1.000 | 0.789 | 99.16 | T |
| | | | | T = 0.002 | T = 0.000 | | | |
| 1436527 | 63616 | 87435716 | C/T | C = 0.709 | C = 0.75 | 0.155 | 1.23 | T |
| | | | | T = 0.291 | T = 0.250 | | | |
| 1436529 | 65377 | 87437477 | T/C | T = 0.448 | T = 0.463 | 0.642 | 1.06 | C |
| | | | | C = 0.552 | C = 0.537 | | | |
| 3775180 | 65442 | 87437542 | A/C | A = 1.000 | A = 1.000 | 0.957 | 4.50 | C |
| | | | | C = 0.000 | C = 0.000 | | | |
| 3775181 | 65548 | 87437648 | T/G | T = 0.758 | T = 0.729 | 0.36 | 0.86 | T |
| | | | | G = 0.242 | G = 0.271 | | | |
| 3775182 | 65878 | 87437978 | T/G | T = 0.143 | T = 0.13 | 0.592 | 0.90 | T |
| | | | | G = 0.857 | G = 0.870 | | | |
| 3775183 | 66222 | 87438322 | G/A | G = 0.446 | G = 0.392 | 0.13 | 0.80 | G |
| | | | | A = 0.554 | A = 0.608 | | | |
| 3775184 | 66354 | 87438454 | A/G | A = 0.828 | A = 0.818 | 0.715 | 0.94 | A |
| | | | | G = 0.172 | G = 0.182 | | | |
| 733245 | 67224 | 87439324 | A/T | A = 0.002 | A = 0.003 | 0.87 | 2.02 | T |
| | | | | T = 0.998 | T = 0.997 | | | |
| 3775185 | 68198 | 87440298 | T/G | T = 0.003 | T = 0.001 | 0.846 | 0.32 | T |
| | | | | G = 0.997 | G = 0.999 | | | |
| 1561154 | 68729 | 87440829 | T/C | T = 0.009 | T = 0.008 | 0.918 | 0.88 | T |
| | | | | C = 0.991 | C = 0.992 | | | |
| 3775186 | 69058 | 87441158 | A/C | A = 0.005 | A = 0.005 | 0.981 | 0.95 | A |
| | | | | C = 0.995 | C = 0.995 | | | |
| 3775187 | 69527 | 87441627 | T/C | T = 0.698 | T = 0.71 | 0.692 | 1.06 | C |
| | | | | C = 0.302 | C = 0.290 | | | |
| 1010778 | 70774 | 87442874 | A/G | A = 0.668 | A = 0.724 | 0.0721 | 1.31 | G |
| | | | | G = 0.332 | G = 0.276 | | | |
| 2282596 | 71232 | 87443332 | T/A | T = 0.265 | T = 0.267 | 0.94 | 1.01 | A |
| | | | | A = 0.735 | A = 0.733 | | | |
| 2282595 | 71943 | 87444043 | A/C | A = 1.000 | A = 1.000 | 0.984 | 0.00 | A |
| | | | | C = 0.000 | C = 0.000 | | | |
| 2118044 | 73397 | 87445497 | A/T | A = 0.656 | A = 0.681 | 0.444 | 1.12 | T |
| | | | | T = 0.344 | T = 0.319 | | | |
| 1469869 | 76322 | 87448422 | C/T | C = 0.615 | C = 0.666 | 0.103 | 1.24 | T |
| | | | | T = 0.385 | T = 0.334 | | | |
| MAPK10-AK | 110704 | 87482804 | A/G | A = 0.526 | A = 0.488 | 0.236 | 0.86 | A |
| | | | | G = 0.474 | G = 0.512 | | | |

FIG. 1B shows proximal SNPs in and around the MAPK10 region for females. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1B can be determined by consulting Table 19. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Web at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 6

### KIAA0861 Proximal SNPs

It has been discovered that a polymorphic variation (rs2001449) in a gene encoding KIAA0861 is associated with the occurrence of breast cancer (see Examples 1 and 2). Subsequently, SNPs proximal to the incident SNP (rs2001449) were identified and allelotyped in breast cancer sample sets and control sample sets as described in Examples 1 and 2. A total of seventy-five allelic variants located within or nearby the KIAA0861 gene were identified and fifty-severn allelic variants were allelotyped. The polymorphic variants are set forth in Table 20. The chromosome position provided in column four of Table 20 is based on Genome “Build 34” of NCBI's GenBank.

TABLE 20

| | Position in | | | | | |
|---|---|---|---|---|---|---|
| | SEQ ID | | Chromosome | Allele | Genome | Deduced |
| dbSNP rs# | NO: 3 | Chromosome | Position | Variants | Letter | Iupac |
| 3811728 | 246 | 3 | 184201246 | t/c | c | Y |
| 3811729 | 393 | 3 | 184201393 | a/g | a | R |
| 602646 | 628 | 3 | 184201628 | c/g | c | S |
| 488277 | 7586 | 3 | 184208586 | t/c | c | Y |
| 1629673 | 9223 | 3 | 184210223 | a/g | g | R |
| 670232 | 9933 | 3 | 184210933 | a/t | a | W |
| 575326 | 10154 | 3 | 184211154 | t/c | c | Y |
| 575386 | 10175 | 3 | 184211175 | c/g | c | S |
| 684846 | 10877 | 3 | 184211877 | t/c | c | Y |
| 471365 | 10907 | 3 | 184211907 | g/c | g | S |
| 496251 | 11289 | 3 | 184212289 | g/a | a | R |
| 831246 | 11793 | 3 | 184212793 | t/c | t | Y |
| 831247 | 11813 | 3 | 184212813 | g/c | g | S |
| KIAA0861-AA | 13507 | 3 | 184214507 | c/g | g | S |
| 512071 | 14249 | 3 | 184215249 | c/t | c | Y |
| 1502761 | 14586 | 3 | 184215586 | a/c | a | M |
| 681516 | 14647 | 3 | 184215647 | c/t | t | Y |
| 683302 | 15004 | 3 | 184216004 | c/t | t | Y |
| 619424 | 16573 | 3 | 184217573 | t/g | g | K |
| 620722 | 16811 | 3 | 184217811 | a/g | a | R |
| 529055 | 18921 | 3 | 184219921 | a/g | a | R |
| 664010 | 19651 | 3 | 184220651 | t/g | g | K |
| 678454 | 20565 | 3 | 184221565 | c/t | c | Y |
| 2653845 | 25239 | 3 | 184226239 | g/a | a | R |
| 472795 | 25721 | 3 | 184226721 | g/a | a | R |
| 507079 | 27133 | 3 | 184228133 | g/a | g | R |
| 534333 | 27778 | 3 | 184228778 | t/c | t | Y |
| 535298 | 27906 | 3 | 184228906 | t/c | t | Y |
| 536213 | 28000 | 3 | 184229000 | g/a | a | R |
| 831245 | 30005 | 3 | 184231005 | a/g | g | R |
| 639690 | 30520 | 3 | 184231520 | t/c | c | Y |
| 684174 | 32195 | 3 | 184233195 | t/c | c | Y |
| 571761 | 32439 | 3 | 184233439 | c/g | c | S |
| 1983421 | 33858 | 3 | 184234858 | t/c | t | Y |
| 4630966 | 41716 | 3 | 184242716 | c/t | t | Y |
| 2314415 | 42450 | 3 | 184243450 | t/g | c | M |
| 6788196 | 43554 | 3 | 184244554 | g/a | g | R |
| 2103062 | 44211 | 3 | 184245211 | a/g | g | R |
| 9827084 | 44775 | 3 | 184245775 | g/c | c | S |
| 9864865 | 44962 | 3 | 184245962 | a/g | a | R |
| 6804951 | 45317 | 3 | 184246317 | c/t | t | Y |
| 6770548 | 45712 | 3 | 184246712 | a/g | a | R |
| 1403452 | 45941 | 3 | 184246941 | t/c | c | Y |
| 7609994 | 46520 | 3 | 184247520 | g/t | t | K |
| 9838250 | 47175 | 3 | 184248175 | c/t | c | Y |
| 9863404 | 48045 | 3 | 184249045 | g/t | t | K |
| 903950 | 48636 | 3 | 184249636 | c/a | t | K |
| 6787284 | 48689 | 3 | 184249689 | g/a | g | R |
| 2017340 | 48704 | 3 | 184249704 | a/g | c | Y |
| 2001449 | 48849 | 3 | 184249849 | g/c | g | S |
| 1317288 | 48850 | 3 | 184249850 | g/a | g | R |
| 7635891 | 49931 | 3 | 184250931 | t/g | g | K |
| 10704581 | 51510 | 3 | 184252510 | —/tt | t | N |
| 11371910 | 51526 | 3 | 184252526 | —/a | c | N |
| 10937118 | 51758 | 3 | 184252758 | a/g | a | R |
| 7642053 | 51975 | 3 | 184252975 | c/g | g | S |
| 3821522 | 53475 | 3 | 184254475 | a/g | c | Y |
| 2029926 | 55524 | 3 | 184256524 | t/c | g | R |
| 1390831 | 56754 | 3 | 184257754 | t/g | a | M |
| 7643890 | 57473 | 3 | 184258473 | a/g | g | R |
| 11925606 | 57497 | 3 | 184258497 | a/c | c | M |
| 9826325 | 57613 | 3 | 184258613 | g/a | a | R |
| 6800429 | 58023 | 3 | 184259023 | g/a | g | R |
| 6803368 | 58821 | 3 | 184259821 | t/c | c | Y |
| 1353566 | 59644 | 3 | 184260644 | c/a | g | K |
| 2272115 | 66217 | 3 | 184267217 | g/a | a | R |
| 2272116 | 66344 | 3 | 184267344 | g/a | g | R |
| 3732603 | 67326 | 3 | 184268326 | g/c | c | S |
| 940055 | 69777 | 3 | 184270777 | a/c | a | M |
| 2314730 | 83594 | 3 | 184284594 | a/g | g | R |
| 2030578 | 84579 | 3 | 184285579 | g/c | g | S |
| 2049280 | 85623 | 3 | 184286623 | c/t | t | Y |
| 3732602 | 126831 | 3 | 184327831 | c/t | a | R |
| 2293203 | 137878 | 3 | 184338878 | a/t | t | W |
| 7639705 | 147455 | 3 | 184348455 | g/t | t | K |

### Assay for Verifying and Allelotyping SNPs

The methods used to verify and allelotype the seventy-five proximal SNPs of Table 20 are the same methods described in Examples 1 and 2 herein. The PCR primers and extend primers used in these assays are provided in Table 21 (SEQ ID NOS 955-1098, respectively, in order of appearance) and Table 22 (SEQ ID NOS 1099-1170, respectively, in order of appearance), respectively.

TABLE 21

| | | |
|---|---|---|
| dbSNP rs# | Forward PCR primer | Reverse PCR primer |

| | | |
|---|---|---|
| 3811728 | ACGTTGGATGACGTGTCGGTCCCCTTTCAT | ACGTTGGATGACGCGCCACACCTCCCTAC |
| 3811729 | ACGTTGGATGTGGGCGAGGTTCTGCAGCGT | ACGTTGGATGGTTTCGTTTCTCCGGCACAG |
| 602646 | ACGTTGGATGGAGGAGACCCAGGGTATGAG | ACGTTGGATGTCTGGGACCGTTTACCGCA |
| 488277 | ACGTTGGATGCACACATTCTTCTCAAGTGC | ACGTTGGATGGGAGGGACACAATTTAACTC |
| 1629673 | ACGTTGGATGGGCACCATGTGTGGCTAATT | ACGTTGGATGAAGGATCACGTGAAGTCAGG |
| 670232 | ACGTTGGATGGAAGGTGGAGCAGACATTAG | ACGTTGGATGACCTTAGTTATACCAGGCAC |
| 575326 | ACGTTGGATGACAGAGAGGCTTGGTCATAC | ACGTTGGATGGGTGCTTGGTTGTGATTCTC |
| 575386 | ACGTTGGATGATTCCTGCAGGTACTGTGTC | ACGTTGGATGTGAGCCCAAAACTACTGCTG |
| 684846 | ACGTTGGATGACCACCAGATAAAATCCCTC | ACGTTGGATGAAGTTCCTCTGGTGGACAAC |
| 471365 | ACGTTGGATGTGAGTGACATTTGTGTCACC | ACGTTGGATGCGGAGGATCTGAACAACTTC |
| 496251 | ACGTTGGATGGGGAGTCATTCCAATACCAG | ACGTTGGATGGGAGTGAAAGGTCATATTGG |
| 831246 | ACGTTGGATGCACAATCTGTTAGAATGGTGG | ACGTTGGATGCGTCAAGACTGAATGCATAG |
| 831247 | ACGTTGGATGGAAAATATAGTCCTACACAA | ACGTTGGATGCGTCAAGACTGAATGCATAG |
| KIAA0861-AA | ACGTTGGATGGTTCTAATGTCACCCCTTCC | ACGTTGGATGCAATGTGGCAAATTCTCTGG |
| 512071 | ACGTTGGATGCAAATCACCCCTGACAATTC | ACGTTGGATGACCAGCACACTCAGCTTTAG |
| 1502761 | ACGTTGGATGCAGAAATATGAAGGTGGCCC | ACGTTGGATGACCTTGAGCTCTGAGCCCTT |
| 681516 | ACGTTGGATGCTCCTCCTCAGAGGACTAAC | ACGTTGGATGAGCCCAAGGACTCATACAAC |
| 683302 | ACGTTGGATGAAACATGGCGAAACCCGGTC | ACGTTGGATGACCACGCCTGGCTAATTTTG |
| 619424 | ACGTTGGATGACCGGGAGCTCCCAGTCTG | ACGTTGGATGTGGGAATCGGTTGAGAGCCG |
| 620722 | ACGTTGGATGGCAGCAAAGAATTGCCCGGC | ACGTTGGATGTAAGGCGCCTGCAGAGGCGA |
| 529055 | ACGTTGGATGCTGCAGTTATCTGGGTGAGC | ACGTTGGATGCCAGAACGTGGCTTGTTGGG |
| 664010 | ACGTTGGATGTGGTACCTCCAGGTAAAATG | ACGTTGGATGTCCAGGCAGTCATTTTACCC |
| 678454 | ACGTTGGATGTTCTCTGCGGAGGAAAGTGC | ACGTTGGATGTTAAGCCAGTCCCCACAAGG |
| 2653845 | ACGTTGGATGATCACTTGGACTCAGGAAGC | ACGTTGGATGAGTCTTGCTCTGTTTCCAGG |
| 472795 | ACGTTGGATGTCACCTGAGCATCAGACATG | ACGTTGGATGATAGTGGAAGGAGAAACGGG |
| 507079 | ACGTTGGATGAAGCCTCAGATGAGGCATAC | ACGTTGGATGTCTGAAAGGGTTCAGGAAGG |
| 534333 | ACGTTGGATGCGTTGATGCACTGAAGGGAG | ACGTTGGATGAGAGGCTAAATGTTGGCAGG |
| 535298 | ACGTTGGATGCAATTGCTCAGACCTTCACC | ACGTTGGATGAATGCTAGAGACATTGCACC |
| 536213 | ACGTTGGATGTGAGGACCTCATTATTGGTG | ACGTTGGATGCTGAGCAATCGAACTGCTAC |
| 831245 | ACGTTGGATGCTAGAATTACAGGTGCACAC | ACGTTGGATGGCCAAGATGGTGAAACCTTG |
| 639690 | ACGTTGGATGGCATTTTACCACCATGTGGTT | ACGTTGGATGCCTTCATGTTAATTCTGCCC |
| 684174 | ACGTTGGATGCTTTACTGAGTGGGCAAACG | ACGTTGGATGTCTAAGTGGAACTCAGCAGC |
| 571761 | ACGTTGGATGAATATCCTAGGCTAGCAGTG | ACGTTGGATGGTGCATAAATACATGAATAG |
| 1983421 | ACGTTGGATGTCCAGGTGTTATGGAGTCAG | ACGTTGGATGGGCTTCTTGTGCTGCTGTGT |
| 4630966 | ACGTTGGATGTCAACAAAGATGCCAAGACC | ACGTTGGATGGTGGATATCCATTGTCCTAG |
| 2314415 | ACGTTGGATGGGCTGAGTAACAGTCCATTG | ACGTTGGATGCTTACAGTATCCAAAAAGGG |
| 6788196 | ACGTTGGATGTCAAAGGTAGGTTACCCCTG | ACGTTGGATGATCCCCAATTTGCACATCCC |
| 2103062 | ACGTTGGATGTGCAGCCCTCAACCTTTCAG | ACGTTGGATGCCTTATTCAGTTACTATTACG |
| 9827084 | ACGTTGGATGAAACACACACACCCACATAC | ACGTTGGATGGGGAGAAAGAAAACAAAGGC |
| 9864865 | ACGTTGGATGCAATGCCTGCACTTAGACAC | ACGTTGGATGAGTGATGAGAACATGGGCTG |
| 6804951 | ACGTTGGATGGCAATAGGACTCCCTTTACC | ACGTTGGATGAAGATACGAATGGAGCCTGG |
| 6770548 | ACGTTGGATGTTTTTGAGCTTCACTGAGCG | ACGTTGGATGCGTATCTCTAGCTCAAGCAT |
| 1403452 | ACGTTGGATGCAGAAGTTAGGATGCAGATG | ACGTTGGATGCCAGTAGAGATAGAATTTTGG |
| 7609994 | ACGTTGGATGATACCTAGAGTTTGCCCAAC | ACGTTGGATGAGCTGAGATCAATCCCTATG |
| 9838250 | ACGTTGGATGGTGGCAGTCAAAACACAGTC | ACGTTGGATGACAGAGTAAGACTCCGTCTC |
| 9863404 | ACGTTGGATGGCTATTAGAAAGTCAGAGCC | ACGTTGGATGTGTTCCAGAAGGTGTAGAAG |
| 903950 | ACGTTGGATGCTTCAGTTCAGGGAGAGATC | ACGTTGGATGATAGGGCCCCCAGCATAAAA |
| 6787284 | ACGTTGGATGGCTTTCCCCTAAAGCATCTC | ACGTTGGATGGATCTCTCCCTGAACTGAAG |
| 2017340 | ACGTTGGATGTATTCCACTGCCTGCTTTCC | ACGTTGGATGGAAAACAGGAGGAAGTGGTG |
| 2001449 | ACGTTGGATGATGTCAAGTGCACCCACATG | ACGTTGGATGAGGAAGAAACTGACGGAAGG |
| 1317288 | ACGTTGGATGATGTCAAGTGCACCCACATG | ACGTTGGATGAGGAAGAAACTGACGGAAGG |
| 7635891 | ACGTTGGATGTCTCACCTTGCCTTTGGACG | ACGTTGGATGCTGATGTCGCAAGGAACCAC |
| 10704581 | ACGTTGGATGCCTGTTGAATTATGGAGGAG | ACGTTGGATGCTCTTCTTTCCATGGATCTTC |
| 11371910 | ACGTTGGATGCTCTTCTTTCCATGGATCTTC | ACGTTGGATGCAGCTAATTTCTCCTGACAG |
| 10937118 | ACGTTGGATGATGCAAACTGGCTGGGAATG | ACGTTGGATGGAGGAGGCTGTGAGAAAAGA |
| 7642053 | ACGTTGGATGATGCCCTGGATTGACCTAAC | ACGTTGGATGGGGTTAGGGTGTGTATAAGG |
| 3821522 | ACGTTGGATGAACCCGCACTACAAGATTCC | ACGTTGGATGGTCAGTCCCACATTCAGAAC |
| 2029926 | ACGTTGGATGTCCCGAACATAAAGACTCAG | ACGTTGGATGGGTTGTAATTGGAACATTGG |
| 1390831 | ACGTTGGATGGTCTGCCAAAGTTCCCTTAG | ACGTTGGATGAGGAAAGGGAAGAGAAACCG |
| 7643890 | ACGTTGGATGGACTGTGAGTTATAGGATAC | ACGTTGGATGATGGGTCGGAGGATTTATAG |
| 11925606 | ACGTTGGATGCTCGGCTAAGGTACTCAATA | ACGTTGGATGAGACCACCAAGTAAAATTGC |
| 9826325 | ACGTTGGATGTTGGGTTAATGCAGGGTCTG | ACGTTGGATGCTAGTTCACCTGGGTCTATC |
| 6800429 | ACGTTGGATGCCAAAGCCCATGTTTTAAAAA | ACGTTGGATGGTTTTTCTAAAATATGGGCT |
| 6803368 | ACGTTGGATGAAACCAGCTCAGGCCATTAC | ACGTTGGATGATGCAAAATAAGCTCTGCCC |
| 1353566 | ACGTTGGATGGGTGTACTCTGCCATTTGTC | ACGTTGGATGTGGAGGAGGTTCTAGTACCC |
| 2272115 | ACGTTGGATGAGTTGTGAGTGATTTCAGGG | ACGTTGGATGCAGGCCTTCTTGCTCTTATC |
| 2272116 | ACGTTGGATGCTGTGCCTTCTGAGTAGTTC | ACGTTGGATGATCTGTTGCCTTAGGTTCAC |
| 3732603 | ACGTTGGATGCTCTCAATTCCATCAGTCTC | ACGTTGGATGCTTTACGAATTTCACAACAGG |
| 940055 | ACGTTGGATGTATGCTTCCAGTCTCTGACC | ACGTTGGATGATAGGTAATCCAGTTGGGCC |
| 2314730 | ACGTTGGATGCTCAGGTAATCTGCCTTCTC | ACGTTGGATGCAGGGATAATGAGAACAAATC |
| 2030578 | ACGTTGGATGAACAACCTTACTTCATGCCC | ACGTTGGATGTTCTCCACTTTCTGGTCAAC |
| 2049280 | ACGTTGGATGTGGATACTGAGGGTCAACTG | ACGTTGGATGCTTCCCAACATTTTCGGCTC |

TABLE 22

| dbSNP rs# | Extend Primer | Term Mix |
|---|---|---|
| 3811728 | GTCCCCTTTCATCTAAAC | ACT |
| 3811729 | TCTGCAGCGTGCGGCGA | ACT |
| 602646 | CCAGGGTATGAGCGGAGGA | ACT |
| 488277 | AGTGCACACAGAACATTTAACA | ACT |
| 1629673 | TGTGGAGACAAGGTCTCACT | ACT |
| 670232 | TGGGCAAACAAGCCCAT | CGT |
| 575326 | TGGTCATACCCTTCAAG | ACT |
| 575386 | GAAGGGTATGACCAAGC | ACT |
| 684846 | AGTTGTTCAGATCCTCC | ACT |
| 471365 | TCCAAAACCACCAGATAAAATC | ACT |
| 496251 | GTATTGTCCTCCAGTGA | ACG |
| 831246 | AGAATGGTGGTGTATTTTTAC | ACT |
| 831247 | TAGTCCTACACAATCTGTTA | ACT |
| KIAA0861-AA | GGTATCAGGAAGAGTCA | ACT |
| 512071 | CCCTGACAATTCCAAAACTAA | ACG |
| 1502761 | GGAGGAGGCACTATTAAT | ACT |
| 681516 | GGCCACCTTCATATTTC | ACG |
| 683302 | CAGGAGATCCAGACCATCC | ACG |
| 619424 | TGCGGCCCCCGCCGGGTT | ACT |
| 620722 | GAATTGCCCGGCTCCGAAT | ACT |
| 529055 | GAGCAGGCAGCACAAGT | ACT |
| 664010 | ACCTCCAGGTAAAATGATTAGTT | ACT |
| 678454 | CAGGGATGGTAATTGAC | ACG |
| 2653845 | AAGCGGAGGTTGCAGTGAGC | ACG |
| 472795 | GACATGTCCCTCTCGGCCT | ACG |
| 507079 | GGCAATGTTTGCCCTTT | ACG |
| 534333 | GGGAGAAAGTAACAGGGTC | ACT |
| 535298 | CAGGTGGATGGGGACAC | ACT |
| 536213 | TGGTGTTAAGTGGCGTG | ACG |
| 831245 | CACACCACCACGCCCGGCT | ACT |
| 639690 | CTGCTATTCATTTGTGTAGA | ACT |
| 684174 | CTCTGATGTTACCTCCTCC | ACT |
| 571761 | CTAGGCTAGCAGTGGGGTTG | ACT |
| 1983421 | GGCAGGGAAGAGAAGAGC | ACT |
| 4630966 | AGATGCCAAGACCATTCAAAG | ACG |
| 2314415 | TAGTTGATGAAGATTTGGG | ACT |
| 6788196 | AGGTTACCCCTGCTGACTTT | ACG |
| 2103062 | GAGATCATTTCTCCTTCAAC | ACT |
| 9827084 | CCACACCCATATATATTTATGCT | ACT |
| 9864865 | AAAGATACACCGTTGAGAAGG | ACT |
| 6804951 | GACTCCCTTTACCTTCATGG | ACG |
| 6770548 | CTTCACTGAGCGTGGTGCC | ACT |
| 1403452 | CACAGATGCTCATGGGTCC | ACT |
| 7609994 | GTTTGCCCAACATATAAACAATAA | CGT |
| 9838250 | AACACAGTCAAAATTTTGCTTCA | ACG |
| 9863404 | GAGCCAAGTTTACATCAAGTTTA | CGT |
| 903950 | AGATCACATTGCCAACCCCCA | CGT |
| 6787284 | CCCGTCTCCTGCTGGTCA | ACG |
| 2017340 | CCCTAAAGCATCTCACAGCCCC | ACT |
| 2001449 | CACATGCCTGCTCGCCCCC | ACT |
| 1317288 | CACATGCCTGCTCGCCCC | ACG |
| 7635891 | TGCCTTTGGACGTCTAGCC | ACT |
| 10704581 | TATGGAGGAGTAGATATTGGAA | CGT |
| 11371910 | GAAAATTCCAATATCTACTCCTC | CGT |
| 10937118 | CTGGGAATGAAATTAGGGCAG | ACT |
| 7642053 | GTTCCCTTGACTTTCCTCAG | ACT |
| 3821522 | GCATCTTCAGGAATCTTG | ACT |
| 2029926 | CATAAAGACTCAGCATTCAGC | ACT |
| 1390831 | GGTTAGGAAGAAATCTGTG | ACT |
| 7643890 | ATCTAGATAATAAAGACCACCAA | ACT |
| 11925606 | CTATTAATGGTGTTTGTCTATGG | ACT |
| 9826325 | TAATGCAGGGTCTGCTGGAT | ACG |
| 6800429 | ATCTCTAAGATATAACACTCTAC | ACG |
| 6803368 | GTGCCTGCAAAGAAAGGAAC | ACT |
| 1353566 | TTGTCAGTTATGAGACCTTG | CGT |
| 2272115 | ATACCTCAGAATACAGCTTTTTTT | ACG |
| 2272116 | TCTCATTTCTCCTCTCTTTC | ACG |
| 3732603 | CTCATTTCCACCCTTCT | ACT |
| 940055 | GTCTCTGACCACTTGACCCA | ACT |
| 2314730 | TCCTTCTTCTCTGCTTT | ACT |
| 2030578 | TCATGCCCATTGGGTTAG | ACT |
| 2049280 | GGGTCAACTGTACCAAG | ACG |

### Genetic Analysis of Allelotyping Results

Allelotyping results are shown for cases and controls in Table 23. The allele frequency for the A2 allele is noted in the fifth and sixth columns for breast cancer pools and control pools, respectively, where “AF” is allele frequency. SNPs with blank allele frequencies were untyped (“not AT”).

TABLE 23

| | Position in | | | | | | | Breast Cancer |
|---|---|---|---|---|---|---|---|---|
| | SEQ ID | Chromosome | A1/A2 | | | | | Associated |
| dbSNP rs# | NO: 3 | Position | Allele | Case AF | Control AF | p-Value | OR | Allele |
| 3811728 | 246 | 184201246 | T/C | T = 0.002 | T = 0.003 | 0.952 | 1.28 | C |
| | | | | C = 0.998 | C = 0.997 | | | |
| 3811729 | 393 | 184201393 | A/G | A = 0.968 | A = 0.947 | 0.268 | 0.61 | A |
| | | | | G = 0.032 | G = 0.053 | | | |
| 602646 | 628 | 184201628 | C/G | C = | C = 0.344 | | | |
| | | | | G= | G = 0.656 | | | |
| 488277 | 7586 | 184208586 | T/C | T = 0.9 | T = 0.898 | 0.92 | 0.98 | T |
| | | | | C = 0.100 | C = 0.102 | | | |
| 1629673 | 9223 | 184210223 | A/G | A = 0.93 | A = 0.911 | 0.459 | 0.78 | A |
| | | | | G = 0.070 | G = 0.089 | | | |
| 670232 | 9933 | 184210933 | A/T | A = 0.138 | A = 0.137 | 0.951 | 0.99 | A |
| | | | | T = 0.862 | T = 0.863 | | | |
| 575326 | 10154 | 184211154 | T/C | T = 0.876 | T = 0.869 | 0.753 | 0.94 | T |
| | | | | C = 0.124 | C = 0.131 | | | |
| 575386 | 10175 | 184211175 | C/G | C = 0.224 | C = 0.221 | 0.921 | 0.98 | C |
| | | | | G = 0.776 | G = 0.779 | | | |
| 684846 | 10877 | 184211877 | T/C | T = 0.202 | T = | | | |
| | | | | C = 0.798 | C = | | | |
| 471365 | 10907 | 184211907 | G/C | G = 0.258 | G = 0.262 | 0.88 | 1.02 | C |
| | | | | C = 0.742 | C = 0.738 | | | |
| 496251 | 11289 | 184212289 | G/A | G = 0.841 | G = 0.839 | 0.967 | 0.99 | G |
| | | | | A = 0.159 | A = 0.161 | | | |
| 831246 | 11793 | 184212793 | T/C | T = 0.229 | T = 0.203 | 0.373 | 0.86 | T |
| | | | | C = 0.771 | C = 0.797 | | | |
| 831247 | 11813 | 184212813 | G/C | G = 0.17 | G = 0.178 | 0.755 | 1.06 | C |
| | | | | C = 0.830 | C = 0.822 | | | |
| KIAA0861- | 13507 | 184214507 | C/G | C = 0.745 | C = 0.762 | 0.557 | 1.10 | G |
| AA | | | | G = 0.255 | G = 0.238 | | | |
| 512071 | 14249 | 184215249 | C/T | C = 0.391 | C = 0.363 | 0.376 | 0.89 | C |
| | | | | T = 0.609 | T = 0.637 | | | |
| 1502761 | 14586 | 184215586 | A/C | A = 0.417 | A = 0.409 | 0.799 | 0.97 | A |
| | | | | C = 0.583 | C = 0.591 | | | |
| 681516 | 14647 | 184215647 | C/T | C = 0.762 | C = 0.817 | 0.0906 | 1.39 | T |
| | | | | T = 0.238 | T = 0.183 | | | |
| 683302 | 15004 | 184216004 | C/T | C = 0.729 | C = | | | |
| | | | | T = 0.271 | T = | | | |
| 619424 | 16573 | 184217573 | T/G | T = 0.925 | T = 0.929 | 0.812 | 1.06 | G |
| | | | | G = 0.075 | G = 0.071 | | | |
| 620722 | 16811 | 184217811 | A/G | T = | A = 0.181 | | | |
| | | | | G= | G = 0.819 | | | |
| 529055 | 18921 | 184219921 | A/G | A = 0.398 | A = 0.364 | 0.325 | 0.86 | A |
| | | | | G = 0.602 | G = 0.636 | | | |
| 664010 | 19651 | 184220651 | T/G | T = 0.549 | T = 0.607 | 0.145 | 1.27 | G |
| | | | | G = 0.451 | G = 0.393 | | | |
| 678454 | 20565 | 184221565 | C/T | C = 1.000 | C = 0.985 | 0.0998 | 0.00 | C |
| | | | | T = 0.000 | T = 0.015 | | | |
| 2653845 | 25239 | 184226239 | G/A | G = 0.825 | G = 0.827 | 0.94 | 1.01 | A |
| | | | | A = 0.175 | A = 0.173 | | | |
| 472795 | 25721 | 184226721 | G/A | G = 0.921 | G = 0.921 | 0.983 | 0.99 | G |
| | | | | A = 0.079 | A = 0.079 | | | |
| 507079 | 27133 | 184228133 | G/A | G = 0.166 | G = 0.167 | 0.979 | 1.00 | A |
| | | | | A = 0.834 | A = 0.833 | | | |
| 534333 | 27778 | 184228778 | T/C | T = 0.502 | T = 0.491 | 0.73 | 0.96 | T |
| | | | | C = 0.498 | C = 0.509 | | | |
| 535298 | 27906 | 184228906 | T/C | T = 0.275 | T = 0.228 | 0.127 | 0.78 | T |
| | | | | C = 0.725 | C = 0.772 | | | |
| 536213 | 28000 | 184229000 | G/A | G = 0.726 | G = 0.717 | 0.781 | 0.96 | G |
| | | | | A = 0.274 | A = 0.283 | | | |
| 831245 | 30005 | 184231005 | A/G | A = 0.979 | A = 0.981 | 0.843 | 1.12 | G |
| | | | | G = 0.021 | G = 0.019 | | | |
| 639690 | 30520 | 184231520 | T/C | T = 0.882 | T = 0.892 | 0.65 | 1.10 | C |
| | | | | C = 0.118 | C = 0.108 | | | |
| 684174 | 32195 | 184233195 | T/C | T = 0.698 | T = 0.708 | 0.756 | 1.05 | C |
| | | | | C = 0.302 | C = 0.292 | | | |
| 571761 | 32439 | 184233439 | C/G | C = 0.601 | C = 0.576 | 0.499 | 0.90 | C |
| | | | | G = 0.399 | G = 0.424 | | | |
| 1983421 | 33858 | 184234858 | T/C | T = 0.566 | T = 0.58 | 0.669 | 1.06 | C |
| | | | | C = 0.434 | C = 0.420 | | | |
| 4630966 | 41716 | 184242716 | C/T | C = 0.359 | C = 0.271 | 0.00247 | 0.66 | C |
| | | | | T = 0.641 | T = 0.729 | | | |
| 2314415 | 42450 | 184243450 | T/G | T = 0.974 | T = 0.951 | 0.124 | 0.53 | T |
| | | | | G = 0.026 | G = 0.049 | | | |
| 6788196 | 43554 | 184244554 | G/A | G = 1.000 | G = 1.000 | 0.967 | 0.00 | G |
| | | | | A = 0 | A = 0.000 | | | |
| 2103062 | 44211 | 184245211 | A/G | A = 0.674 | A = 0.642 | 0.381 | 0.87 | A |
| | | | | G = 0.326 | G = 0.358 | | | |
| 9827084 | 44775 | 184245775 | G/C | G = 0.966 | G = 0.928 | 0.0403 | 0.46 | G |
| | | | | C = 0.034 | C = 0.072 | | | |
| 9864865 | 44962 | 184245962 | A/G | A = 0.106 | A = 0.185 | 0.000529 | 1.93 | G |
| | | | | G = 0.894 | G = 0.815 | | | |
| 6804951 | 45317 | 184246317 | C/T | C = 0.96 | C = 0.904 | 0.00573 | 0.40 | C |
| | | | | T = 0.040 | T = 0.096 | | | |
| 6770548 | 45712 | 184246712 | A/G | A = 0.062 | A = 0.159 | 1.12E−05 | 2.86 | G |
| | | | | G = 0.938 | G = 0.841 | | | |
| 1403452 | 45941 | 184246941 | T/C | T = 0.97 | T = 0.932 | 0.0144 | 0.43 | T |
| | | | | C = 0.030 | C = 0.068 | | | |
| 7609994 | 46520 | 184247520 | G/T | G = 0.001 | G = 0.002 | 0.918 | 2.34 | T |
| | | | | T = 0.999 | T = 0.998 | | | |
| 9838250 | 47175 | 184248175 | C/T | C = 0.52 | C = 0.524 | 0.909 | 1.01 | T |
| | | | | T = 0.480 | T = 0.476 | | | |
| 9863404 | 48045 | 184249045 | G/T | G = 0.001 | G = 0.002 | 0.887 | 2.58 | T |
| | | | | T = 0.999 | T = 0.998 | | | |
| 903950 | 48636 | 184249636 | C/A | C = 0.417 | C = 0.406 | 0.739 | 0.96 | C |
| | | | | A = 0.583 | A = 0.594 | | | |
| 6787284 | 48689 | 184249689 | G/A | G = 0.475 | G = 0.501 | 0.416 | 1.11 | A |
| | | | | A = 0.525 | A = 0.499 | | | |
| 2017340 | 48704 | 184249704 | A/G | A = 0.965 | A = 0.945 | 0.195 | 0.63 | A |
| | | | | G = 0.035 | G = 0.055 | | | |
| 2001449 | 48849 | 184249849 | G/C | G = 0.738 | G = 0.797 | 0.0285 | 1.39 | C |
| | | | | C = 0.262 | C = 0.203 | | | |
| 1317288 | 48850 | 184249850 | G/A | G = 1.000 | G = 1.000 | 0.967 | 0.51 | G |
| | | | | A = 0.000 | A = 0.000 | | | |
| 7635891 | 49931 | 184250931 | T/G | T = 0.973 | T = 0.947 | 0.121 | 0.49 | T |
| | | | | G = 0.027 | G = 0.053 | | | |
| 10704581 | 51510 | 184252510 | —/TT | — = 0.998 | — = 0.997 | 0.949 | 0.83 | — |
| | | | | TT = 0.002 | TT = 0.003 | | | |
| 11371910 | 51526 | 184252526 | —/A | — = 1.000 | — = 1.000 | 0.977 | 0.00 | — |
| | | | | A = 0 | A = 0.000 | | | |
| 10937118 | 51758 | 184252758 | A/G | A = 0.495 | A = 0.51 | 0.629 | 1.06 | G |
| | | | | G = 0.505 | G = 0.490 | | | |
| 7642053 | 51975 | 184252975 | C/G | C = 0.002 | C = 0.003 | 0.908 | 1.85 | G |
| | | | | G = 0.998 | G = 0.997 | | | |
| 3821522 | 53475 | 184254475 | A/G | A = 0.504 | A = 0.52 | 0.62 | 1.07 | G |
| | | | | G = 0.496 | G = 0.480 | | | |
| 2029926 | 55524 | 184256524 | T/C | T = 0.001 | T = 0.001 | 0.975 | 2.52 | C |
| | | | | C = 0.999 | C = 0.999 | | | |
| 1390831 | 56754 | 184257754 | T/G | T = 0.057 | T = 0.076 | 0.284 | 1.36 | G |
| | | | | G = 0.943 | G = 0.924 | | | |
| 7643890 | 57473 | 184258473 | A/G | A = 0.001 | A = 0.002 | 0.934 | 2.30 | G |
| | | | | G = 0.999 | G = 0.998 | | | |
| 11925606 | 57497 | 184258497 | A/C | A = 0 | A = 0.001 | 0.956 | | C |
| | | | | C = 1.000 | C = 0.999 | | | |
| 9826325 | 57613 | 184258613 | G/A | G = 0.002 | G = 0.003 | 0.887 | 1.85 | A |
| | | | | A = 0.998 | A = 0.997 | | | |
| 6800429 | 58023 | 184259023 | G/A | G = 0.605 | G = 0.59 | 0.662 | 0.94 | G |
| | | | | A = 0.395 | A = 0.410 | | | |
| 6803368 | 58821 | 184259821 | T/C | T = 0.002 | T = 0.001 | 0.885 | 0.20 | T |
| | | | | C = 0.998 | C = 0.999 | | | |
| 1353566 | 59644 | 184260644 | C/A | C = 0.452 | C = 0.469 | 0.604 | 1.07 | A |
| | | | | A = 0.548 | A = 0.531 | | | |
| 2272115 | 66217 | 184267217 | G/A | G = 0.673 | G = 0.634 | 0.224 | 0.84 | G |
| | | | | A = 0.327 | A = 0.366 | | | |
| 2272116 | 66344 | 184267344 | G/A | G = 0.999 | G = 1.000 | 0.876 | 8.14 | A |
| | | | | A = 0.001 | A = 0.000 | | | |
| 3732603 | 67326 | 184268326 | G/C | G = 0.773 | G = 0.792 | 0.495 | 1.11 | C |
| | | | | C = 0.227 | C = 0.208 | | | |
| 940055 | 69777 | 184270777 | A/C | A = 0.778 | A = 0.803 | 0.356 | 1.17 | C |
| | | | | C = 0.222 | C = 0.197 | | | |
| 2314730 | 83594 | 184284594 | A/G | A = 0.352 | A = 0.311 | 0.183 | 0.83 | A |
| | | | | G = 0.648 | G = 0.689 | | | |
| 2030578 | 84579 | 184285579 | G/C | G = 1.000 | G = 1.000 | 0.963 | 0.33 | G |
| | | | | C = 0.000 | C = 0.000 | | | |
| 2049280 | 85623 | 184286623 | C/T | C = 0.001 | C = 0.005 | 0.576 | 30.94 | T |
| | | | | T = 0.999 | T = 0.995 | | | |

FIG. 1C shows proximal SNPs in and around the KIAA0861 gene for females. As indicated, some of the SNPs were untyped. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1C can be determined by consulting Table 23. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Wed at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

Additional Genotyping

A total of fourteen SNPs, including the incident SNP, were genotyped in the discovery cohort. The discovery cohort is described in Example 1. Four of the SNPs are non-synonomous, coding SNPs. Two of the SNPs (rs2001449 and rs6804951) were found to be significantly associated with breast cancer with a p-value of 0.001 and 0.007, respectively. See Table 26.

The methods used to verify and genotype the five proximal SNPs of Table 26 are the same methods described in Examples 1 and 2 herein. The PCR primers and extend primers used in these assays are provided in Table 24 (SEQ ID NOS 1171-1198, respectively, in order of appearance) and Table 25 (SEQ ID NOS 1199-1212, respectively, in order of appearance), respectively.

TABLE 24

| | | |
|---|---|---|
| dbSNP # | Forward PCR primer | Revee PCR primer |

| | | |
|---|---|---|
| 7639705 | ACGTTGGATGTGTCAGAAAGCAAACCTGGC | ACGTTGGATGTTACAGGCATTGGAGACAGC |
| 2293203 | ACGTTGGATGCTGCATAATGGTGGCTTTGG | ACGTTGGATGTGTGGGTGTTCACTTTGCAG |
| 3732602 | ACGTTGGATGCCCTCTTGTCAGGAAGTTCT | ACGTTGGATGGAGACAGAGTTGAACTCCCG |
| 2001449 | ACGTTGGATGAGGAAGAAACTGACGGAAGG | ACGTTGGATGATGTCAAGTGCACCCACATG |
| 6804951 | ACGTTGGATGAAGATACGAATGGAGCCTGG | ACGTTGGATGGCAATAGGACTCCCTTTACC |
| 3821522 | ACGTTGGATGCGCACTACAAGATTCCAAGC | ACGTTGGATGTCAGTCCCACATTCAGAACC |
| 2293203 | ACGTTGGATGTGTGGGTGTTCACTTTGCAG | ACGTTGGATGCTGCATAATGGTGGCTTTGG |
| 3811729 | ACGTTGGATGTGGGCGAGGTTCTGCAGCGT | ACGTTGGATGGTTTCGTTTCTCCGGCACAG |
| 534333 | ACGTTGGATGGATGCACTGAAGGGAGAAAG | ACGTTGGATGAGAGGCTAAATGTTGGCAGG |
| 575326 | ACGTTGGATGTGAGCCCAAAACTACTGCTG | ACGTTGGATGATTCCTGCAGGTACTGTGTC |
| 2272115 | ACGTTGGATGCAGGCCTTCTTGCTCTTATC | ACGTTGGATGAGTTGTGAGTGATTTCAGGG |
| 940055 | ACGTTGGATGTATGCTTCCAGTCTCTGACC | ACGTTGGATGGATAGGTAATCCAGTTGGGC |
| 2017340 | ACGTTGGATGGATCTCTCCCTGAACTGAAG | ACGTTGGATGGCTTTCCCCTAAAGCATCTC |
| 571761 | ACGTTGGATGAATATCCTAGGCTAGCAGTG | ACGTTGGATGGTGCATAAATACATGAATAG |

TABLE 25

| dbSNP # | Extend Primer | Term Mix |
|---|---|---|
| 7639705 | TGATGCACGTGGAGCAG | CGT |
| 2293203 | GCCCCTGGAAAAGGCCC | CGT |
| 3732602 | GGAAGATGATGAGACTAAAT | ACG |
| 2001449 | CACATGCCTGCTCGCCCCC | ACT |
| 6804951 | TCCCTTTACCTTCATGG | ACG |
| 3821522 | GCATCTTCAGGAATCTTG | ACT |
| 2293203 | GCCCCTGGAAAAGGCCC | CGT |
| 3811729 | GGTTCTGCAGCGTGCGGCGA | ACT |
| 534333 | GAAGGGAGAAAGTAACAGGGTC | ACT |
| 575326 | TGGTCATACCCTTCAAG | ACT |
| 2272115 | ATCTTCTACACATTGATTCAG | ACT |
| 940055 | TCTCTGACCACTTGACCCA | ACT |
| 2017340 | TGGTGACCAGCAGGAGA | ACG |
| 571761 | GGCTAGCAGTGGGGTTG | ACT |

Table 26, below, shows the case and control allele frequencies along with the p-values for all of the SNPs genotyped. The disease associated allele of column 4 is in bold and the disease associated amino acid of column 5 is also in bold. The chromosome positions provided correspond to NCBI's Build 34. The amino acid change positions provided in column 5 correspond to KIAA0861 polypeptide sequence of SEQ ID NO: 22. The corresponding amino acid position in the alternative KIAA0861 polypeptide sequence (NP_{—}055893) can be easily calculated by adding 83 amino acids to the positions provided in column 5.

TABLE 26
Genotyping Results

| | | | | | | | | | Breast |
|---|---|---|---|---|---|---|---|---|---|
| | Position in | | | Amino | | | | | Cancer |
| | SEQ ID | Chromosome | Alleles | Acid | | AF F | | Odds | Associated |
| dbSNP rs# | NO: 3 | Position | (A1/A2) | Change | AF F case | control | p-value | Ratio | Allele |
| 3811729 | 393 | 184201393 | A/G | | A = 0.917 | A = 0.948 | 0.0542 | 1.65 | G |
| | | | | | G = 0.083 | G = 0.052 | | | |
| 575326 | 10154 | 184211154 | T/C | | T = 0.897 | T = 0.885 | 0.545 | 0.88 | T |
| | | | | | C = 0.103 | C = 0.115 | | | |
| 534333 | 27778 | 184228778 | T/C | | T = 0.254 | T = 0.249 | 0.85 | 0.97 | T |
| | | | | | C = 0.746 | C = 0.751 | | | |
| 571761 | 32439 | 184233439 | C/G | | C = 0.487 | C = 0.465 | 0.492 | 0.92 | C |
| | | | | | G = 0.513 | G = 0.535 | | | |
| 6804951 | 45317 | 184246317 | C/T | A819T | C = 0.956 | C = 0.915 | 0.007 | 2.02 | C |
| | | | | | T = 0.044 | T = 0.085 | | | |
| 2017340 | 48704 | 184249704 | G/A | | G = 0.027 | G = 0.042 | 0.203 | 1.57 | A |
| | | | | | A = 0.973 | A = 0.958 | | | |
| 2001449 | 48849 | 184249849 | G/C | | G = 0.693 | G = 0.782 | 0.001 | 1.59 | C |
| | | | | | C = 0.307 | C = 0.218 | | | |
| 3821522 | 53475 | 184254475 | A/G | | A = 0.372 | A = 0.391 | 0.539 | 1.08 | G |
| | | | | | G = 0.628 | G = 0.609 | | | |
| 2272115 | 66217 | 184267217 | A/G | | A = 0.407 | A = 0.444 | 0.246 | 1.16 | G |
| | | | | | G = 0.593 | G = 0.556 | | | |
| 940055 | 69777 | 184270777 | A/C | | A = 0.702 | A = 0.753 | 0.0721 | 1.29 | C |
| | | | | | C = 0.298 | C = 0.247 | | | |
| 3732602 | 126831 | 184327831 | C/T | S506F | C = 0.008 | C = 0.012 | 0.597 | 1.41 | T |
| | | | | | T = 0.992 | T = 0.988 | | | |
| 2293203 | 137878 | 184338878 | A/T | L295Q | A = 0.012 | A = 0.015 | 0.690 | 1.24 | T |
| | | | | | T = 0.988 | T = 0.985 | | | |
| 7639705 | 147455 | 184348455 | G/T | I276L | G = 0.195 | G = 0.189 | 0.794 | 1.04 | G |
| | | | | | T = 0.805 | T = 0.811 | | | |

### Example 7

### NUMA1 Region Proximal SNPs

It has been discovered that a polymorphic variation (rs673478) in the NUMA1/FLJ20625/LOC220074 region is associated with the occurrence of breast cancer (see Examples 1 and 2). Subsequently, SNPs proximal to the incident SNP (rs673478) were identified and allelotyped in breast cancer sample sets and control sample sets as described in Examples 1 and 2. Approximately 327 allelic variants located within the NUMA1/FLJ20625/LOC220074 region were identified and allelotyped. The polymorphic variants are set forth in Table 27. The chromosome position provided in column four of Table 27 is based on Genome “Build 34” of NCBI's GenBank.

TABLE 27

| | Position in | | | Allele | Genome | Deduced |
|---|---|---|---|---|---|---|
| dbSNP rs# | SEQ ID NO: 4 | Chromosome | Chromosome Position | Variants | Letter | Iupac |
| 4945392 | 203 | 11 | 71420953 | t/c | c | Y |
| 7938496 | 1236 | 11 | 71421986 | g/a | a | R |
| 7926550 | 1768 | 11 | 71422518 | c/g | g | S |
| 7945374 | 2101 | 11 | 71422851 | a/t | t | W |
| 7949480 | 3146 | 11 | 71423896 | t/c | t | Y |
| 7102523 | 4476 | 11 | 71425226 | t/c | t | Y |
| 7121260 | 6124 | 11 | 71426874 | a/g | g | R |
| 7131230 | 6719 | 11 | 71427469 | c/t | t | Y |
| 7128317 | 6925 | 11 | 71427675 | g/a | a | R |
| 2276385 | 7060 | 11 | 71427810 | c/g | g | S |
| 2276384 | 7081 | 11 | 71427831 | t/c | g | R |
| 2276383 | 7090 | 11 | 71427840 | a/g | c | Y |
| 1892921 | 7696 | 11 | 71428446 | c/t | g | R |
| 1892920 | 7989 | 11 | 71428739 | c/t | a | R |
| 7942626 | 9407 | 11 | 71430157 | g/a | g | R |
| 7124429 | 9699 | 11 | 71430449 | t/g | g | K |
| 7114081 | 9905 | 11 | 71430655 | a/t | t | W |
| 7125718 | 9984 | 11 | 71430734 | c/t | c | Y |
| 1055452 | 13023 | 11 | 71433773 | t/c | t | Y |
| 3829215 | 13047 | 11 | 71433797 | g/a | t | Y |
| 1541306 | 13571 | 11 | 71434321 | c/t | g | R |
| 3814722 | 14086 | 11 | 71434836 | c/a | g | K |
| 1939240 | 14337 | 11 | 71435087 | t/a | t | W |
| 5743655 | 14462 | 11 | 71435212 | t/c | c | Y |
| 5743656 | 14518 | 11 | 71435268 | a/g | g | R |
| 5743657 | 14695 | 11 | 71435445 | a/g | g | R |
| 3814721 | 14830 | 11 | 71435580 | g/a | c | Y |
| 5743658 | 15282 | 11 | 71436032 | c/a | a | M |
| 5743659 | 15436 | 11 | 71436186 | a/g | g | R |
| 5743660 | 15745 | 11 | 71436495 | g/a | g | R |
| 5743661 | 15748 | 11 | 71436498 | g/t | g | K |
| 5743662 | 15797 | 11 | 71436547 | t/c | t | Y |
| 2298455 | 16036 | 11 | 71436786 | t/g | a | M |
| 5743664 | 16646 | 11 | 71437396 | g/a | g | R |
| 5743665 | 16713 | 11 | 71437463 | a/g | a | R |
| 5743667 | 16859 | 11 | 71437609 | g/a | g | R |
| 5743668 | 16914 | 11 | 71437664 | g/— | c | N |
| 5743669 | 17422 | 11 | 71438172 | c/t | c | Y |
| 1573503 | 17499 | 11 | 71438249 | a/g | t | Y |
| 5743670 | 17646 | 11 | 71438396 | g/c | c | S |
| 5743671 | 17681 | 11 | 71438431 | g/a | g | R |
| 1892919 | 17733 | 11 | 71438483 | c/t | g | R |
| 5743672 | 17841 | 11 | 71438591 | g/a | g | R |
| 2735786 | 17963 | 11 | 71438713 | a/g | g | R |
| 5743673 | 18011 | 11 | 71438761 | g/a | g | R |
| 5743674 | 18238 | 11 | 71438988 | a/t | a | W |
| 1062452 | 18569 | 11 | 71439319 | c/a | c | M |
| 5743675 | 18672 | 11 | 71439422 | t/c | c | Y |
| 5743676 | 18715 | 11 | 71439465 | a/g | g | R |
| 760246 | 18927 | 11 | 71439677 | c/t | t | Y |
| 11345794 | 19266 | 11 | 71440016 | t/— | t | N |
| 5743677 | 19290 | 11 | 71440040 | c/t | c | Y |
| 5743678 | 19456 | 11 | 71440206 | t/g | t | K |
| 14537 | 19511 | 11 | 71440261 | c/t | g | R |
| 3168177 | 19575 | 11 | 71440325 | g/a | c | Y |
| 5743679 | 19591 | 11 | 71440341 | g/a | g | R |
| 1541304 | 19636 | 11 | 71440386 | g/a | t | Y |
| 1053725 | 19770 | 11 | 71440520 | a/g | c | Y |
| 5743680 | 20002 | 11 | 71440752 | g/a | a | R |
| 949324 | 20084 | 11 | 71440834 | g/c | g | S |
| 949323 | 20086 | 11 | 71440836 | a/c | t | K |
| 5743681 | 20246 | 11 | 71440996 | t/c | c | Y |
| 5743682 | 20253 | 11 | 71441003 | t/c | t | Y |
| 5743683 | 20300 | 11 | 71441050 | g/a | g | R |
| 5743684 | 20539 | 11 | 71441289 | g/a | g | R |
| 5743685 | 20682 | 11 | 71441432 | c/t | c | Y |
| 2845857 | 20773 | 11 | 71441523 | g/c | c | S |
| 2852365 | 20776 | 11 | 71441526 | g/a | t | Y |
| 11538641 | 20904 | 11 | 71441654 | c/a | g | K |
| 11538639 | 20918 | 11 | 71441668 | c/t | g | R |
| 11235417 | 20978 | 11 | 71441728 | a/g | g | R |
| 11538644 | 21269 | 11 | 71442019 | c/a | g | K |
| 11538643 | 21354 | 11 | 71442104 | c/t | g | R |
| 2845858 | 21488 | 11 | 71442238 | t/c | c | Y |
| 2155146 | 21656 | 11 | 71442406 | g/t | a | M |
| 2852364 | 21793 | 11 | 71442543 | t/c | g | R |
| 2845859 | 22084 | 11 | 71442834 | a/g | a | R |
| 2845860 | 22219 | 11 | 71442969 | t/c | c | Y |
| 11538642 | 22815 | 11 | 71443565 | g/t | c | M |
| 7949430 | 22858 | 11 | 71443608 | t/c | c | Y |
| 7938674 | 22895 | 11 | 71443645 | t/c | t | Y |
| 2735787 | 23431 | 11 | 71444181 | c/t | t | Y |
| 11538640 | 23968 | 11 | 71444718 | c/t | a | R |
| 10736784 | 24469 | 11 | 71445219 | t/a | t | W |
| 10736785 | 24470 | 11 | 71445220 | a/c | c | M |
| 11826059 | 24853 | 11 | 71445603 | t/c | t | Y |
| 2503 | 24961 | 11 | 71445711 | t/c | t | Y |
| 2155145 | 25177 | 11 | 71445927 | a/c | g | K |
| 4477459 | 26174 | 11 | 71446924 | t/c | t | Y |
| NUMA1-AA | 26334 | 11 | 71447084 | t/— | t | N |
| 5019605 | 26337 | 11 | 71447087 | c/g | c | S |
| 5019604 | 26338 | 11 | 71447088 | a/t | t | W |
| 5019603 | 26339 | 11 | 71447089 | c/g | c | S |
| 3793941 | 26965 | 11 | 71447715 | g/a | c | Y |
| 11235418 | 27278 | 11 | 71448028 | c/g | c | S |
| 3793940 | 28042 | 11 | 71448792 | a/g | c | Y |
| 2032353 | 28520 | 11 | 71449270 | g/t | c | M |
| 3934448 | 28533 | 11 | 71449283 | g/t | c | M |
| 7951267 | 28764 | 11 | 71449514 | a/g | g | R |
| 1053603 | 29685 | 11 | 71450435 | g/a | c | Y |
| 1053602 | 30738 | 11 | 71451488 | a/g | c | Y |
| 11235419 | 31175 | 11 | 71451925 | t/c | c | Y |
| 1053601 | 31490 | 11 | 71452240 | a/g | c | Y |
| 3750913 | 31726 | 11 | 71452476 | g/c | g | S |
| 3750912 | 31770 | 11 | 71452520 | a/g | c | Y |
| 1053600 | 31792 | 11 | 71452542 | t/a | t | W |
| 1057992 | 32980 | 11 | 71453730 | c/a | a | M |
| 2298789 | 33398 | 11 | 71454148 | a/g | t | Y |
| 10898813 | 33580 | 11 | 71454330 | t/a | t | W |
| 11278712 | 34468-34472 | 11 | 71455218\hat{ }71455222 | —/gtcaac | gtcaac | NNNNN |
| NUMA1-AB | 34941 | 11 | 71455691 | c/t | t | Y |
| TA 949325 | 35214 | 11 | 71455964 | g/a | a | R |
| NUMA1-AC | 35629 | 11 | 71456379 | g/c | c | S |
| 949326 | 35864 | 11 | 71456614 | t/c | c | Y |
| 2298456 | 36273 | 11 | 71457023 | a/t | t | W |
| 7949845 | 37281 | 11 | 71458031 | a/g | a | R |
| 7949989 | 37378 | 11 | 71458128 | a/g | a | R |
| 1573500 | 38348 | 11 | 71459098 | c/g | c | S |
| 4945411 | 38608 | 11 | 71459358 | a/g | g | R |
| 3831387 | 38627-38628 | 11 | 71459377\hat{ }71459378 | ca/— | ca | NN |
| 11235422 | 39049 | 11 | 71459799 | c/a | c | M |
| 10128658 | 39077 | 11 | 71459827 | c/t | c | Y |
| 2298457 | 39769 | 11 | 71460519 | t/c | t | Y |
| 3838779 | 40555 | 11 | 71461305 | t/— | c | N |
| 1573501 | 41251 | 11 | 71462001 | t/c | c | Y |
| 11235424 | 41270 | 11 | 71462020 | c/t | c | Y |
| 10898814 | 42063 | 11 | 71462813 | t/c | t | Y |
| 7930142 | 42574 | 11 | 71463324 | t/c | c | Y |
| 7930544 | 42912 | 11 | 71463662 | c/t | c | Y |
| 7930721 | 43039 | 11 | 71463789 | a/c | c | M |
| 7930722 | 43042 | 11 | 71463792 | c/g | c | S |
| 6592456 | 43221 | 11 | 71463971 | a/g | g | R |
| 11235425 | 43518 | 11 | 71464268 | t/c | c | Y |
| 6592457 | 43644 | 11 | 71464394 | g/c | g | S |
| 10898815 | 44981 | 11 | 71465731 | a/g | g | R |
| 11235426 | 45013 | 11 | 71465763 | t/c | c | Y |
| 10898816 | 45038 | 11 | 71465788 | c/t | c | Y |
| 1939247 | 46079 | 11 | 71466829 | t/a | a | W |
| 1939246 | 47848 | 11 | 71468598 | t/g | a | M |
| 1939245 | 48090 | 11 | 71468840 | a/g | c | Y |
| 1939244 | 48147 | 11 | 71468897 | g/t | c | M |
| 1939243 | 48293 | 11 | 71469043 | c/t | g | R |
| 4245463 | 48771 | 11 | 71469521 | c/a | c | M |
| 4944258 | 48951 | 11 | 71469701 | a/t | t | W |
| 7926751 | 49972 | 11 | 71470722 | t/g | t | K |
| 12282917 | 50161 | 11 | 71470911 | g/a | g | R |
| 12282918 | 50163 | 11 | 71470913 | g/a | g | R |
| 7937582 | 50171 | 11 | 71470921 | t/c | c | Y |
| 10898817 | 51289 | 11 | 71472039 | t/c | c | Y |
| 7480015 | 51699 | 11 | 71472449 | g/a | a | R |
| 7123992 | 52005 | 11 | 71472755 | c/t | c | Y |
| 1573502 | 52986 | 11 | 71473736 | g/a | c | Y |
| 7127865 | 53339 | 11 | 71474089 | g/c | g | S |
| 4945426 | 53704 | 11 | 71474454 | c/a | c | M |
| 11235428 | 54615 | 11 | 71475365 | g/c | g | S |
| 7122489 | 55307 | 11 | 71476057 | a/c | a | M |
| 1894003 | 55834 | 11 | 71476584 | t/c | a | R |
| 1548348 | 56475 | 11 | 71477225 | g/a | a | R |
| 1892923 | 57053 | 11 | 71477803 | g/a | c | Y |
| 5792570 | 57355 | 11 | 71478105 | —/g | g | N |
| 7115200 | 57718 | 11 | 71478468 | t/g | t | K |
| 4945430 | 58703 | 11 | 71479453 | g/c | g | S |
| 11235429 | 58772 | 11 | 71479522 | c/t | t | Y |
| 1939242 | 59140 | 11 | 71479890 | c/t | g | R |
| 9666346 | 59450 | 11 | 71480200 | g/c | c | S |
| 7101553 | 59461 | 11 | 71480211 | t/c | c | Y |
| 7124000 | 59896 | 11 | 71480646 | c/t | t | Y |
| 12291664 | 60157 | 11 | 71480907 | g/a | g | R |
| 12291778 | 60369 | 11 | 71481119 | a/g | g | R |
| 12291781 | 60395 | 11 | 71481145 | a/g | g | R |
| 12291787 | 60424 | 11 | 71481174 | g/a | g | R |
| 12291788 | 60426 | 11 | 71481176 | g/a | g | R |
| 12291833 | 60456 | 11 | 71481206 | a/g | g | R |
| 10793016 | 60621 | 11 | 71481371 | g/a | g | R |
| 5792571 | 61207 | 11 | 71481957 | g/— | g | N |
| 12293529 | 61209 | 11 | 71481959 | g/c | g | S |
| 4338555 | 61254 | 11 | 71482004 | c/t | t | Y |
| 11235431 | 61854 | 11 | 71482604 | c/g | g | S |
| 6592458 | 63103 | 11 | 71483853 | a/g | g | R |
| 4945434 | 64645 | 11 | 71485395 | c/t | c | Y |
| 4945435 | 64777 | 11 | 71485527 | t/c | c | Y |
| 11307657 | 64980 | 11 | 71485730 | t/— | t | N |
| 5792573 | 64987 | 11 | 71485737 | t/— | t | N |
| 1894004 | 65375 | 11 | 71486125 | t/c | g | R |
| 12276164 | 65561 | 11 | 71486311 | a/g | g | R |
| 4378421 | 67554 | 11 | 71488304 | c/t | t | Y |
| 7116495 | 67643 | 11 | 71488393 | c/t | c | Y |
| 7101701 | 67713 | 11 | 71488463 | a/g | a | R |
| 10736786 | 68832 | 11 | 71489582 | t/c | c | Y |
| 11608165 | 70184 | 11 | 71490934 | t/g | t | K |
| 645603 | 70415 | 11 | 71491165 | g/a | a | R |
| 661290 | 71572 | 11 | 71492322 | a/g | a | R |
| 7122209 | 71732 | 11 | 71492482 | t/c | t | Y |
| 12417471 | 72829 | 11 | 71493579 | a/g | a | R |
| 541228 | 73154 | 11 | 71493904 | c/g | c | S |
| 12273666 | 73221 | 11 | 71493971 | a/g | a | R |
| 2511074 | 74343 | 11 | 71495093 | t/c | g | R |
| 3018311 | 74462 | 11 | 71495212 | t/g | g | K |
| 3018289 | 74471 | 11 | 71495221 | t/c | t | Y |
| 679926 | 75494 | 11 | 71496244 | a/g | c | Y |
| 567026 | 75510 | 11 | 71496260 | g/a | t | Y |
| 564294 | 75827 | 11 | 71496577 | g/a | t | Y |
| 678193 | 75831 | 11 | 71496581 | t/g | a | M |
| 677279 | 76027 | 11 | 71496777 | t/c | g | R |
| 560777 | 76160 | 11 | 71496910 | c/t | a | R |
| 676721 | 76196 | 11 | 71496946 | c/t | g | R |
| 7106529 | 76378 | 11 | 71497128 | a/t | a | W |
| 11602304 | 77306 | 11 | 71498056 | g/a | g | R |
| 585228 | 78847 | 11 | 71499597 | c/g | g | S |
| 578957 | 78918 | 11 | 71499668 | c/a | a | M |
| 7110215 | 79117 | 11 | 71499867 | a/g | g | R |
| 3133233 | 79811 | 11 | 71500561 | c/g | g | S |
| 3133230 | 79843 | 11 | 71500593 | t/c | c | Y |
| 12576024 | 80448 | 11 | 71501198 | g/t | g | K |
| 674319 | 80949 | 11 | 71501699 | c/t | t | Y |
| 675185 | 81130 | 11 | 71501880 | t/g | t | K |
| 5792574 | 81452 | 11 | 71502202 | —/c | a | N |
| 12275272 | 82595 | 11 | 71503345 | c/t | c | Y |
| 10400327 | 83633 | 11 | 71504383 | c/t | t | Y |
| 612255 | 84222 | 11 | 71504972 | c/t | a | R |
| 7101643 | 84378 | 11 | 71505128 | g/a | g | R |
| 575871 | 84380 | 11 | 71505130 | a/g | t | Y |
| 547208 | 85226 | 11 | 71505976 | c/t | a | R |
| 2511075 | 85815 | 11 | 71506565 | t/c | t | Y |
| 642573 | 86412 | 11 | 71507162 | c/g | g | S |
| 482197 | 86805 | 11 | 71507555 | g/a | a | R |
| 656640 | 87268 | 11 | 71508018 | t/c | t | Y |
| 671681 | 88370 | 11 | 71509120 | c/t | t | Y |
| 541022 | 88614 | 11 | 71509364 | a/g | g | R |
| 951586 | 89223 | 11 | 71509973 | a/g | c | Y |
| 2511076 | 89385 | 11 | 71510135 | g/a | a | R |
| 11235432 | 89500 | 11 | 71510250 | g/c | g | S |
| 3018308 | 89502 | 11 | 71510252 | t/c | t | Y |
| 11606798 | 89929 | 11 | 71510679 | g/c | g | S |
| 1791544 | 90515 | 11 | 71511265 | c/t | g | R |
| 3018304 | 90659 | 11 | 71511409 | c/a | t | K |
| 10751193 | 90914 | 11 | 71511664 | a/c | a | M |
| 4945470 | 91594 | 11 | 71512344 | c/t | t | Y |
| 3018291 | 91603 | 11 | 71512353 | a/c | a | M |
| 2511120 | 91769 | 11 | 71512519 | a/g | c | Y |
| 671132 | 92005 | 11 | 71512755 | g/a | c | Y |
| 4945475 | 93335 | 11 | 71514085 | g/c | g | S |
| 3018292 | 93443 | 11 | 71514193 | t/g | t | K |
| 642618 | 93751 | 11 | 71514501 | c/t | g | R |
| 552966 | 93775 | 11 | 71514525 | a/c | a | M |
| 6592459 | 94351 | 11 | 71515101 | t/g | g | K |
| 607446 | 94810 | 11 | 71515560 | c/t | g | R |
| 607070 | 94856 | 11 | 71515606 | a/g | c | Y |
| 10713307 | 95893 | 11 | 71516643 | c/— | c | N |
| 3018302 | 96500 | 11 | 71517250 | t/g | a | M |
| 3750909 | 97587 | 11 | 71518337 | g/c | g | S |
| 3018301 | 97629 | 11 | 71518379 | a/g | a | R |
| 2511114 | 97705 | 11 | 71518455 | c/t | t | Y |
| 12270166 | 97967 | 11 | 71518717 | a/g | g | R |
| 12270241 | 98126 | 11 | 71518876 | g/a | g | R |
| 11606587 | 98422 | 11 | 71519172 | a/g | a | R |
| 686340 | 98770 | 11 | 71519520 | g/c | c | S |
| 548961 | 99445 | 11 | 71520195 | g/a | g | R |
| 549032 | 99467 | 11 | 71520217 | c/a | a | M |
| 575831 | 100104 | 11 | 71520854 | a/g | a | R |
| 575878 | 100121 | 11 | 71520871 | a/t | t | W |
| 577435 | 100239 | 11 | 71520989 | t/c | c | Y |
| 579320 | 100512 | 11 | 71521262 | g/t | g | K |
| 495567 | 101046 | 11 | 71521796 | c/t | c | Y |
| 636946 | 101267 | 11 | 71522017 | c/t | t | Y |
| 493065 | 102487 | 11 | 71523237 | a/g | t | Y |
| 597513 | 102980 | 11 | 71523730 | a/t | t | W |
| 598835 | 103285 | 11 | 71524035 | t/c | c | Y |
| 10683614 | 103359 | 11 | 71524109 | —/tagt | t | N |
| 610004 | 103497 | 11 | 71524247 | t/c | c | Y |
| 610041 | 103526 | 11 | 71524276 | a/g | a | R |
| 673478 | 104662 | 11 | 71525412 | t/c | g | R |
| 670802 | 105226 | 11 | 71525976 | t/g | a | M |
| 505041 | 105950 | 11 | 71526700 | t/a | t | W |
| 2511116 | 107718 | 11 | 71528468 | c/t | g | R |
| 628025 | 107909 | 11 | 71528659 | a/c | a | M |
| 517837 | 107917 | 11 | 71528667 | c/t | a | R |
| 615000 | 108510 | 11 | 71529260 | t/g | a | M |
| 482013 | 109520 | 11 | 71530270 | c/t | g | R |
| 693391 | 109676 | 11 | 71530426 | t/g | c | M |
| 2511079 | 109712 | 11 | 71530462 | t/c | c | Y |
| 2250866 | 110071 | 11 | 71530821 | t/c | g | R |
| 2508860 | 110610 | 11 | 71531360 | a/g | c | Y |
| 7483267 | 110757 | 11 | 71531507 | a/g | g | R |
| 2511078 | 110758 | 11 | 71531508 | g/a | c | Y |
| 2508859 | 110923 | 11 | 71531673 | t/g | a | M |
| 2508858 | 110963 | 11 | 71531713 | c/g | c | S |
| 11235435 | 111245 | 11 | 71531995 | t/g | g | K |
| 11235436 | 111437 | 11 | 71532187 | a/g | g | R |
| 639435 | 112174 | 11 | 71532924 | a/c | t | K |
| 12285624 | 112646 | 11 | 71533396 | a/g | a | R |
| NUMA1-AD | 112784 | 11 | 71533534 | g/t | t | K |
| 624363 | 113275 | 11 | 71534025 | t/c | g | R |
| NUMA1-AE | 113614 | 11 | 71534364 | t/c | t | Y |
| 1053573 | 113965 | 11 | 71534715 | g/t | a | M |
| 1053511 | 114095 | 11 | 71534845 | c/t | g | R |
| 1063863 | 114125 | 11 | 71534875 | g/a | c | Y |
| 12137 | 114213 | 11 | 71534963 | c/t | c | Y |
| 4365081 | 114278 | 11 | 71535028 | a/g | g | R |
| 4466868 | 114321 | 11 | 71535071 | g/c | g | S |
| 3750911 | 114465 | 11 | 71535215 | a/g | g | R |
| 510925 | 115058 | 11 | 71535808 | t/c | c | Y |
| 595062 | 115193 | 11 | 71535943 | a/g | c | Y |
| 1053443 | 115433 | 11 | 71536183 | c/t | g | R |
| 542752 | 116202 | 11 | 71536952 | a/t | t | W |
| 3897579 | 116328 | 11 | 71537078 | t/c | g | R |
| 11235437 | 116812 | 11 | 71537562 | t/c | c | Y |
| 2508856 | 117201 | 11 | 71537951 | c/t | g | R |
| 5792575 | 117565 | 11 | 71538315 | a/— | a | N |
| 659513 | 117969 | 11 | 71538719 | g/a | g | R |
| 10898820 | 118150 | 11 | 71538900 | a/g | g | R |
| 2276397 | 127729 | 11 | 71548479 | c/t | a | R |
| 3750908 | 127959 | 11 | 71548709 | c/t | a | R |
| 3793938 | 128691 | 11 | 71549441 | c/t | g | R |
| 602285 | 129086 | 11 | 71549836 | c/g | g | S |
| 2276396 | 129463 | 11 | 71550213 | g/c | g | S |
| 2276395 | 129900 | 11 | 71550650 | t/c | g | R |
| 1806778 | 136610 | 11 | 71557360 | t/c | a | R |
| 4073394 | 137797 | 11 | 71558547 | a/g | a | R |
| 471547 | 151737 | 11 | 71572487 | g/t | g | K |
| 606136 | 152130 | 11 | 71572880 | a/g | c | Y |
| 605241 | 152332 | 11 | 71573082 | t/c | g | R |
| 686063 | 153789 | 11 | 71574539 | g/a | t | Y |
| 685749 | 153813 | 11 | 71574563 | t/c | g | R |
| 533207 | 153844 | 11 | 71574594 | a/c | a | M |
| 476753 | 188612 | 11 | 71609362 | a/g | g | R |
| NUMA1-AF | 191149 | 11 | 71611899 | g/t | g | K |

### Assay for Verifying and Allelotyping SNPs

The methods used to verify and allelotype the proximal SNPs of Table 27 are the same methods described in Examples 1 and 2 herein. The PCR primers and extend primers used in these assays are provided in Table 28 (SEQ ID NOS 1213-1866, respectively, in order of appearance) and Table 29 (SEQ ID NOS 1867-2193, respectively, in order of appearance), respectively.

TABLE 28

| | | |
|---|---|---|
| dbSNP rs# | Forward PCR primer | Reverse PCR primer |

| | | |
|---|---|---|
| 4945392 | ACGTTGGATGGGCACACAGTAGGTGCTTAT | ACGTTGGATGCCACCAGCTTGTGAGGTAAT |
| 7938496 | ACGTTGGATGTGCAGTGAACTGCACTCCAG | ACGTTGGATGCTGGCCAGTAACTACTAGCT |
| 7926550 | ACGTTGGATGTTACTGAGTTTCCTAGGCTG | ACGTTGGATGGAATCCCAACACTTTGGGAG |
| 7945374 | ACGTTGGATGGAACACACACAGCTCTATTC | ACGTTGGATGCACATCCTTTTCACCGTGTC |
| 7949480 | ACGTTGGATGGGTTTCACCATATTGGCCAG | ACGTTGGATGCACGCCTATAATCCAAACAC |
| 7102523 | ACGTTGGATGAAACTGGATGTGTTCCTGGG | ACGTTGGATGGTAGACTCAGAGCTTAGGTG |
| 7121260 | ACGTTGGATGGCTACTGATCAAATTAAGTGG | ACGTTGGATGGCTGCATTTGCTTATGAACC |
| 7131230 | ACGTTGGATGAGACAATCTCAGAAAGAGGG | ACGTTGGATGATCCATAAGGAGGAAAGGGC |
| 7128317 | ACGTTGGATGAGACCAAGCTACAGTATCCC | ACGTTGGATGGGTAGCTGCAAGTCATTGTC |
| 2276385 | ACGTTGGATGAAGTGCGGGCAAACTATGAC | ACGTTGGATGTCCTCAAGAGGTGTTCATGG |
| 2276384 | ACGTTGGATGAGACAGCAGACTGGATTTGG | ACGTTGGATGACTACTCTGGGACACACAAG |
| 2276383 | ACGTTGGATGACTACTCTGGGACACACAAG | ACGTTGGATGTTTGGTCATAGTTTGCCCGC |
| 1892921 | ACGTTGGATGCCTGTTGCCCATGTAATGAG | ACGTTGGATGTACAAGAGGAGGAGGGAGTG |
| 1892920 | ACGTTGGATGAGTTTATCTGGGCAAAAACC | ACGTTGGATGAATGCCTGCTATGTGTCTAG |
| 7942626 | ACGTTGGATGGAAATATTTACTTGCGTTTCTC | ACGTTGGATGTGGACATCACGGAGAGATGC |
| 7124429 | ACGTTGGATGCTGGATCAAATTGAAAACAG | ACGTTGGATGGAAGGTAAAGCACAAGACAG |
| 7114081 | ACGTTGGATGTGGGCAGAGTTTCTGTTTGG | ACGTTGGATGGCATTAATTACATTTGCAGTG |
| 7125718 | ACGTTGGATGTCCCAAACAGAAACTCTGCC | ACGTTGGATGAGGCATGTTCACAGAGATGG |
| 1055452 | ACGTTGGATGATCATATCCCTCCCCAGTCC | ACGTTGGATGAAGACCTCCTGGGTGGGAAA |
| 3829215 | ACGTTGGATGTGGGTGGGAAAGACTCAAAG | ACGTTGGATGTCCATCATATCCCTCCCCAG |
| 1541306 | ACGTTGGATGCTGATTCTTCCAGTTGTCCC | ACGTTGGATGACCTTATCAGTGACTTGGCC |
| 3814722 | ACGTTGGATGATCCTGCTTCGTTCTTGCTG | ACGTTGGATGAAGTGGGAGGTCAAACATGG |
| 1939240 | ACGTTGGATGATTCCACCACTCCTAGGGAT | ACGTTGGATGTTAACCCCCATCGGAGACCT |
| 5743655 | ACGTTGGATGTGAGTGTGGTCCTCAGGAAG | ACGTTGGATGCAGACTGAGGTTGCTCTATG |
| 5743656 | ACGTTGGATGACTCAGGCTCGGCTTCTCTT | ACGTTGGATGACAGGTGGCATAAGCAGCAG |
| 5743657 | ACGTTGGATGTGTCAGGCTCAGGTGAGAAG | ACGTTGGATGGAGGTCAAGGGACTCAGAAC |
| 3814721 | ACGTTGGATGGACTGTCTCTCTCAGAAACC | ACGTTGGATGATTCCTCCCAAGAACACCAG |
| 5743658 | ACGTTGGATGCCTCCTTAGATTTAGCATCC | ACGTTGGATGTCACTGATGGGACAAATGGC |
| 5743659 | ACGTTGGATGTTGCCAGAGCCAGTGTTGAG | ACGTTGGATGAGGAACTGGCACACAAGCTC |
| 5743660 | ACGTTGGATGTTCAGGCTCAATGCCATACC | ACGTTGGATGTGGGAGATGTAGCCGACCTT |
| 5743661 | ACGTTGGATGTTCAGGCTCAATGCCATACC | ACGTTGGATGTGGGAGATGTAGCCGACCTT |
| 5743662 | ACGTTGGATGGACAACTTTCCTAGGCACTG | ACGTTGGATGAAAGGCCAGGATGTGGACGG |
| 2298455 | ACGTTGGATGTCCTACTCTCCAGAGGATAC | ACGTTGGATGAGTTCATACCCTAGGTGACC |
| 5743664 | ACGTTGGATGCCCATTGCTCCATTCTGTTC | ACGTTGGATGCATGAGACACAACTGGACAC |
| 5743665 | ACGTTGGATGGAATGGAGCAATGGGCTAAC | ACGTTGGATGGCCCAGGTTCTTAAAGCAAC |
| 5743667 | ACGTTGGATGTAAGGATGTAACTCCAGCCC | ACGTTGGATGGTTCAATAAAGGGCTAAGGG |
| 5743668 | ACGTTGGATGGAGGAAAGGACAGTTACAGG | ACGTTGGATGGGCTGGAGTTACATCCTTAC |
| 5743669 | ACGTTGGATGAGTAATGGTCACAGGACCTC | ACGTTGGATGTGCTCCTCTGAGATGGCCAG |
| 1573503 | ACGTTGGATGGATGACCTTTTCTAGTGCCC | ACGTTGGATGTCAGAGGAGCAGCAGCCATC |
| 5743670 | ACGTTGGATGTAGAGAACTGCTCTGCACAC | ACGTTGGATGCTCACATTCCCCGTGTTTAG |
| 5743671 | ACGTTGGATGCATGCATCTGGAACCTAGAG | ACGTTGGATGACTGCACTTCTGTAACGGAC |
| 1892919 | ACGTTGGATGTAGCTCTTCAGAAGAGCAGG | ACGTTGGATGGTTCTCTAGGTTCCAGATGC |
| 5743672 | ACGTTGGATGATGAAGGAACCATTGCCCAG | ACGTTGGATGATGGAACGCTGAGCTTATCC |
| 2735786 | ACGTTGGATGAGCACCAGGTGAGGGTCGCA | ACGTTGGATGAGGGAAGGAAAGGTCATGAG |
| 5743673 | ACGTTGGATGAGCACCAAGGCCTTGCACAG | ACGTTGGATGTCTCATGACCTTTCCTTCCC |
| 5743674 | ACGTTGGATGTGAGGAATGCTGGGCATTAG | ACGTTGGATGGAGCTCTGCTTCCATATGTG |
| 1062452 | ACGTTGGATGTGTCTACCTGGAGTGAACAG | ACGTTGGATGGACCCAAAGCAGAGAAGGAG |
| 5743675 | ACGTTGGATGGATAGGTGGAGAGAGAATGG | ACGTTGGATGCAAACTCCATTCCCACCTAC |
| 5743676 | ACGTTGGATGCATTCTCTCTCCACCTATCC | ACGTTGGATGAGTCTTGGTGGTTTCTGAGC |
| 760246 | ACGTTGGATGTTTAGTCCCGTCTTCCTCAC | ACGTTGGATGGATGACTTCACTGAAGAGGC |
| 11345794 | ACGTTGGATGCAGAAGCTGAAGACAACACC | ACGTTGGATGGAGCATTATGGGCTGTTCTC |
| 5743677 | ACGTTGGATGAACACAGGCGCTTGAAAAAG | ACGTTGGATGCTCTTCCCACTCTCCATTAG |
| 5743678 | ACGTTGGATGACTCAGCAGGAACAAGGTAG | ACGTTGGATGCTATTTTTCTCCCGTGAGTAC |
| 14537 | ACGTTGGATGTGGGAAGGTTTTTAAGGGCC | ACGTTGGATGAGTCAACTCAGTACTCACGG |
| 3168177 | ACGTTGGATGCCTTCCCATCACTCCAAATC | ACGTTGGATGACAGCCTTCAGCAGCAGCTC |
| 5743679 | ACGTTGGATGATGGGCCCTATGTCTCAGGA | ACGTTGGATGAAATCCCACCCCAGTGCAAG |
| 1541304 | ACGTTGGATGAAGGTAGGGTGTGAGCTGCTG | ACGTTGGATGAGGATGCTCTTTCCCAGGAC |
| 1053725 | ACGTTGGATGTAGCCTCTGGATCTAGAAGG | ACGTTGGATGTCCTGGACTTCAAGAGTGAG |
| 5743680 | ACGTTGGATGAACAGTAGCCAAACCCCTAG | ACGTTGGATGAGAAGGCGCCAGTGAAGGAC |
| 949324 | ACGTTGGATGTGAGAGAAGGCACTGAGAGG | ACGTTGGATGAAGCACTAAAGGGCCAGTAC |
| 949323 | ACGTTGGATGTGAGAGAAGGCACTGAGAGG | ACGTTGGATGAAGCACTAAAGGGCCAGTAC |
| 5743681 | ACGTTGGATGACAGCCAGCAACTGATAACC | ACGTTGGATGCTGGGAAGATCTCTTCCTGC |
| 5743682 | ACGTTGGATGACAGCCAGCAACTGATAACC | ACGTTGGATGCTGGGAAGATCTCTTCCTGC |
| 5743683 | ACGTTGGATGTCTCCAAGGAGAGAAGACAG | ACGTTGGATGTTATCAGTTGCTGGCTGTGC |
| 5743684 | ACGTTGGATGTTCTCCGCGCATTGCCACCA | ACGTTGGATGAGTGCCTCTACCTTGCCCTT |
| 5743685 | ACGTTGGATGCTAGCTTCTTGGGTGTGTTG | ACGTTGGATGTCTGACAGCCCTTCCTTGCA |
| 2845857 | ACGTTGGATGTACTAAACAGGTGAGGCTGG | ACGTTGGATGTTCCCAGGCCAGCGTGCTGT |
| 2852365 | ACGTTGGATGTTCCCAGGCCAGCGTGCTGT | ACGTTGGATGTACTAAACAGGTGAGGCTGG |
| 11538641 | ACGTTGGATGACTCCTGAGTCTAAGAAGGC | ACGTTGGATGTCAGTAGTGCTCTGTTTGCG |
| 11538639 | ACGTTGGATGACTCCTGAGTCTAAGAAGGC | ACGTTGGATGTCTGTTTGCGCCCTTCATGT |
| 11235417 | ACGTTGGATGGGTGGCCTTCTTAGACTATG | ACGTTGGATGAAGACACCTGGCTTCAGATG |
| 11538644 | ACGTTGGATGAGCAGCGCAAACGGGTCTC | ACGTTGGATGGGAAACAGCTGGTGGCCTTC |
| 11538643 | ACGTTGGATGCCATCACAGATGAGGAGATG | ACGTTGGATGCTATCTGGATTGGCTGCATG |
| 2845858 | ACGTTGGATGAAGGGTCCCGACCATAAGTG | ACGTTGGATGTAGCTGTATGGAGATGGAGG |
| 2155146 | ACGTTGGATGGATTCCTCTCTGAGCTCCTG | ACGTTGGATGTGTTTCCAGGTGGAACAGAC |
| 2852364 | ACGTTGGATGCTAGATCAGCAGGTGGGATT | ACGTTGGATGTGAAGACCTGCTATCCCCTG |
| 2845859 | ACGTTGGATGCCACCTACCCCATAAACTTG | ACGTTGGATGATAGTCTGTGTGCCGTACTC |
| 2845860 | ACGTTGGATGCCCGGTCCTTAATTTGTTCC | ACGTTGGATGCACGCAGAGACTGCTACTAC |
| 11538642 | ACGTTGGATGAGGCTAGCCTGGGAAGCAGGA | ACGTTGGATGGCTAGATGTGGAAGAGCCAG |
| 7949430 | ACGTTGGATGGTACACTGCAACAGATTCAC | ACGTTGGATGGCTGTAGAACGATGAGTTGG |
| 7938674 | ACGTTGGATGAAGGGAATGGGAAGCTTCTG | ACGTTGGATGCTAGCTTCTGGAAGACAGAG |
| 2735787 | ACGTTGGATGCAAACCTCCCCTTCAAGATG | ACGTTGGATGATTGTTGCTGCCCAGTGGAC |
| 11538640 | ACGTTGGATGAACCAGCCTCACCTATCTCC | ACGTTGGATGCAGGGATGGGAGTGAAGTAG |
| 10736784 | ACGTTGGATGATCCAATGTCGTCCTGCTTG | ACGTTGGATGAAGGACATGAGCATGGAGTG |
| 10736785 | ACGTTGGATGAAGGACATGAGCATGGAGTG | ACGTTGGATGATCCAATGTCGTCCTGCTTG |
| 11826059 | ACGTTGGATGAGGAACCTTGGAAGAATTTG | ACGTTGGATGGTCTTAACTTCTATACTAGC |
| 2503 | ACGTTGGATGACTAAGCTGAGATGTGAGGC | ACGTTGGATGCACCAAGGTTTCCTCAATGC |
| 2155145 | ACGTTGGATGGATATCTGGTATAATACCCG | ACGTTGGATGTCAGGCTGTATCCTAGTGAG |
| 4477459 | ACGTTGGATGAGAAGGCAAAGATAGGGCAG | ACGTTGGATGCGACTACAGTCTACTAAACC |
| NUMA1-AA | ACGTTGGATGAAGTTCCCCTGTCCTAAGAG | ACGTTGGATGAACCAATCCTCAGGGAACAC |
| 5019605 | ACGTTGGATGTAGGAGGAAGGGAACCAATC | ACGTTGGATGAAGAGAGCTCACATGCCAAG |
| 5019604 | ACGTTGGATGAAGTTCCCCTGTCCTAAGAG | ACGTTGGATGAACCAATCCTCAGGGAACAC |
| 5019603 | ACGTTGGATGAACCAATCCTCAGGGAACAC | ACGTTGGATGAAGTTCCCCTGTCCTAAGAG |
| 3793941 | ACGTTGGATGAAGGTGACACAGTTGAAAGC | ACGTTGGATGTCTAGATAGTCTGGGCAGGG |
| 11235418 | ACGTTGGATGTCTAAGCCACAGGCTTCCTA | ACGTTGGATGGCTATTCTCTAAGCTCTGGG |
| 3793940 | ACGTTGGATGTCTCTCTTTTCCCCTACTCC | ACGTTGGATGGCAGCACATGTACTCCAAAG |
| 2032353 | ACGTTGGATGTAGAGCCCCAGAAAGTGATG | ACGTTGGATGCTTGAAGGTGATATGCGAGG |
| 3934448 | ACGTTGGATGTAGAGCCCCAGAAAGTGATG | ACGTTGGATGCTTGAAGGTGATATGCGAGG |
| 7951267 | ACGTTGGATGATTGGTTCAGGGTGGGAAAG | ACGTTGGATGACCCTTCTGCACCTTCTCTC |
| 1053603 | ACGTTGGATGGTCTCAGCATCAGCACGTAC | ACGTTGGATGGTTTCTGGAAGTGGAGTTGG |
| 1053602 | ACGTTGGATGTGGCAGGACAGAGCCAACAG | ACGTTGGATGTGCTGCTTCTGGCATTGCTG |
| 11235419 | ACGTTGGATGAGGCAGCGCTGCAGGCTATG | ACGTTGGATGGCCCGCAGCCGTTCCAGCT |
| 1053601 | ACGTTGGATGCACAGCGAGCTCCAGATAAG | ACGTTGGATGACTTCCTTCTCTTGGACCTG |
| 3750913 | ACGTTGGATGCACACTCACTCTCAGCTGTG | ACGTTGGATGCCATCAGGCTGAGACTGAAG |
| 3750912 | ACGTTGGATGCTCGATTACAGCAGCTTGGG | ACGTTGGATGTCACTCTCAGCTGTGTGCTGG |
| 1053600 | ACGTTGGATGAAGGAGCTGGAAGAAGAGAG | ACGTTGGATGACTTCAGTCTCAGCCTGATG |
| 1057992 | ACGTTGGATGGAACACCAGGAACCCAAATG | ACGTTGGATGTTGCAGGTAGGAATGCACAG |
| 2298789 | ACGTTGGATGCAAGACTTATCTGAGGAAGG | ACGTTGGATGAACCTGCTTACTACTTCCCC |
| 10898813 | ACGTTGGATGGCACCTACAATAGGCTATGC | ACGTTGGATGTAGAGCGAGACTCTGTCTCA |
| 11278712 | ACGTTGGATGGGCAAGTGAGAATCCCCATC | ACGTTGGATGATAATTGGAGGCATCCAGCC |
| NUMA1-AB | ACGTTGGATGGGGAAGCAGTGAGAAGAGAG | ACGTTGGATGAGCTCTCCCTGCATTCCTTC |
| 949325 | ACGTTGGATGTGTCCCCATCTTCTCTTTCC | ACGTTGGATGCAGAACTATGTGCACAAGGG |
| NUMA1-AC | ACGTTGGATGTAGTGCGCACTTCCATAGAC | ACGTTGGATGCATATGCATTGGTAAGCGCC |
| 949326 | ACGTTGGATGCCCTCAAAAGCTGTTCCCTG | ACGTTGGATGCTGCAGAGATGTGCTAAGAG |
| 2298456 | ACGTTGGATGGTTCTCAAATTGCATAAAGG | ACGTTGGATGTAGAAGGCACAGGAGCTGGG |
| 7949845 | ACGTTGGATGTCACCTGATGTCAGGAGTTC | ACGTTGGATGCCACCACACCTGGCTAATTT |
| 7949989 | ACGTTGGATGAAATTAGCCAGGTGTGGTGG | ACGTTGGATGAATCTCCACTTCCCGTGTTC |
| 1573500 | ACGTTGGATGCTATAACCTCCAACTCCTGG | ACGTTGGATGAAAAATGAGCTGGGTGTGGC |
| 4945411 | ACGTTGGATGGAGGAAAGGATGAGATAAATG | ACGTTGGATGCTCTGTTCCTATATGTGTTTC |
| 3831387 | ACGTTGGATGATGAAGTGAAAGTTCTGTGG | ACGTTGGATGCGTAGAAATTATTCTCTGTTCC |
| 11235422 | ACGTTGGATGTCTAGAAGCCAAACAGGAGG | ACGTTGGATGAAGAGTCACCGGAATAGCTC |
| 10128658 | ACGTTGGATGTCTAGAAGCCAAACAGGAGG | ACGTTGGATGAAGAGTCACCGGAATAGCTC |
| 2298457 | ACGTTGGATGTGGTTTCCAGGAAGTTCACC | ACGTTGGATGTGTGCAGATACCAGGCATTC |
| 3838779 | ACGTTGGATGCTGTTTCCCCGAACTTCAAG | ACGTTGGATGCTTGCAAGGGAAAGAGACTC |
| 1573501 | ACGTTGGATGTAGCTTCCCGTTCATCTGTG | ACGTTGGATGCTGCCACCCTATTTGCAAAC |
| 11235424 | ACGTTGGATGCTGCCACCCTATTTGCAAAC | ACGTTGGATGTTGAGCCATTCCAGATGGTG |
| 10898814 | ACGTTGGATGTGGAACTGCAGTTAGCTAGG | ACGTTGGATGAATACTAGTCCCTGGGATCC |
| 7930142 | ACGTTGGATGACATTTCACCTGAGAACCTC | ACGTTGGATGATGTTGTGACTCCTTGGCTC |
| 7930544 | ACGTTGGATGGCAGCACTCATGAATGCAAC | ACGTTGGATGGCACCTACTTTATTCCTGGC |
| 7930721 | ACGTTGGATGTAGTGGAGGCATTTTGCCAG | ACGTTGGATGGAGGGATGCTTTTTACTGAG |
| 7930722 | ACGTTGGATGGAGGGATGCTTTTTACTGAG | ACGTTGGATGTAGTGGAGGCATTTTGCCAG |
| 6592456 | ACGTTGGATGAAGGGAAACTTGTCAGAGGG | ACGTTGGATGATAAGCTGTGGGAGTTGAGG |
| 11235425 | ACGTTGGATGCTTGGAGGTAGGCGATATTG | ACGTTGGATGAAGGAAGCTGGTCTGCATTG |
| 6592457 | ACGTTGGATGGTGTGTTGTCCTTGGGTTAG | ACGTTGGATGGCATCTTGAATTTTTGGCCC |
| 10898815 | ACGTTGGATGTGAGTCCCTGATACCAGTTC | ACGTTGGATGAGGCCAAGGATAGACTGAAG |
| 11235426 | ACGTTGGATGTTAGATTGTTTGCCCCAGGC | ACGTTGGATGTGATACCAGTTCCTAACTGC |
| 10898816 | ACGTTGGATGTTCAGTCTATCCTTGGCCTG | ACGTTGGATGCCAAGCATAGGTCAGTTGTC |
| 1939247 | ACGTTGGATGAAAGGAAGGGCTCTCTGATG | ACGTTGGATGTAATTTCAGGAAGGAGTGCC |
| 1939246 | ACGTTGGATGCACAGTGCAGGGACACTATC | ACGTTGGATGATAAGAGGCTGAGGTAGGTG |
| 1939245 | ACGTTGGATGTGCGCCTGGCCTAAAAGTTG | ACGTTGGATGTCAATGATTTCTCTACAGAC |
| 1939244 | ACGTTGGATGAACAACTTTTAGGCCAGGCG | ACGTTGGATGTCAAGCAGTCCTTCCACCTC |
| 1939243 | ACGTTGGATGCTCAAGCGATCTTCCCACGT | ACGTTGGATGTTTTTAGCTGGGTGAGGTGG |
| 4245463 | ACGTTGGATGTGAAGCTGCAACTGCATTCC | ACGTTGGATGCTGTTTTAACTGGGATTCATC |
| 4944258 | ACGTTGGATGGTCTCTTCATTTTCTGCAGG | ACGTTGGATGTTGGTCAGGACAGGTCATTG |
| 7926751 | ACGTTGGATGCTGATCCCAGCACATATAAC | ACGTTGGATGATAGAGTGAAAGCCTGTGTC |
| 12282917 | ACGTTGGATGTCAGAAGTTTGCAACCACCC | ACGTTGGATGCCACCACACCCAGCTAATTT |
| 12282918 | ACGTTGGATGTCAGAAGTTTGCAACCACCC | ACGTTGGATGCCACCACACCCAGCTAATTT |
| 7937582 | ACGTTGGATGGAGCTCAGAAGTTTGCAACC | ACGTTGGATGCCACACCCAGCTAATTTTTG |
| 10898817 | ACGTTGGATGTCTCTGACCCAGGGCAAGTT | ACGTTGGATGTTGCAGTGAGCCGAGATCAC |
| 7480015 | ACGTTGGATGTAATCCCAGCACTTTGGGAG | ACGTTGGATGACAGGTTTTTGCCATGTTGC |
| 7123992 | ACGTTGGATGGACCAAAGTCCCTGCATATG | ACGTTGGATGCAGAGCAAGACTCGGTCTAA |
| 1573502 | ACGTTGGATGTGCTGATTTAATGTGACCAG | ACGTTGGATGCATTTTGCAGATAAAGAAA |
| 7127865 | ACGTTGGATGAGGTGGCAGAATCTCAATGG | ACGTTGGATGGGAGATCCCATGGGACATTT |
| 4945426 | ACGTTGGATGACACCATTCTTGCCCAGAAG | ACGTTGGATGTGGATAGGAAGGACCAAGAG |
| 11235428 | ACGTTGGATGAGAAACCCCAGTTGAGTGTC | ACGTTGGATGGCTGACAAGACTAAAGCTCG |
| 7122489 | ACGTTGGATGTCTCAGCAGATCCTTCTGTG | ACGTTGGATGAGTACACTGGGTGATGCTGA |
| 1894003 | ACGTTGGATGGCCTAGCCATGATTTCCTAC | ACGTTGGATGTTGACCCTCTGGTCCTGC |
| 1548348 | ACGTTGGATGTCGGTGTTTGCTTGCATGTG | ACGTTGGATGGTGAAAACCCTGATCTTGGG |
| 1892923 | ACGTTGGATGTGCAGCCATGTATTGTACTG | ACGTTGGATGTGTGTGAATATAGTGGTGGG |
| 5792570 | ACGTTGGATGGAAAGCAACCCTTTGCTCTG | ACGTTGGATGATACTCAGAAACAGGCGCTG |
| 7115200 | ACGTTGGATGTTGGTTTTCTTCTCAGTCTG | ACGTTGGATGAAAGAAAGCAGACTCCTGGC |
| 4945430 | ACGTTGGATGCAGCCATGTAGGAGAAGCAG | ACGTTGGATGCTGCAGCCCTTAACAGACG |
| 11235429 | ACGTTGGATGACTCTGCTTCTCCTACATGG | ACGTTGGATGGAAGTTCGCTATTCAGTGGG |
| 1939242 | ACGTTGGATGCTCCTCTCTGCTTTCTCTTC | ACGTTGGATGCCTTGAAACACACAGGTAGC |
| 9666346 | ACGTTGGATGAAGTGCTGGGATTACAGGTG | ACGTTGGATGTAATGAGGGCACACAAGAGG |
| 7101553 | ACGTTGGATGAAGTGCTGGGATTACAGGTG | ACGTTGGATGTAATGAGGGCACACAAGAGG |
| 7124000 | ACGTTGGATGTAATCCCAGCACTTTGAGAG | ACGTTGGATGAGAACTCCGCCTGGCTAATT |
| 12291664 | ACGTTGGATGGCTGTGCCTAGGCAACATAG | ACGTTGGATGTACAGGTGTGTGCCACCATG |
| 12291778 | ACGTTGGATGGTCACAATCATTGGCTACAG | ACGTTGGATGGGGACTCTGACACTAAAAAAG |
| 12291781 | ACGTTGGATGGTCACAATCATTGGCTACAG | ACGTTGGATGGGGACTCTGACACTAAAAAAG |
| 12291787 | ACGTTGGATGAAGGAATTCTCCCACCCTAG | ACGTTGGATGGTCACAATCATTGGCTACAG |
| 12291788 | ACGTTGGATGAAGGAATTCTCCCACCCTAG | ACGTTGGATGGTCACAATCATTGGCTACAG |
| 12291833 | ACGTTGGATGTTTAGTGTCAGAGTCCCAGC | ACGTTGGATGGGCATGATCTCTGCTCATTC |
| 10793016 | ACGTTGGATGGGTACCTACTTCATTAAGGG | ACGTTGGATGAGCTAATGCTTACTGAGTGC |
| 5792571 | ACGTTGGATGCCTACAATCTAACTCCTCCC | ACGTTGGATGCATCAGTCTGCCTCTCTAGA |
| 12293529 | ACGTTGGATGTCCCTACAATCTAACTCCTC | ACGTTGGATGTTTACTTTAGGGTCCAGCTC |
| 4338555 | ACGTTGGATGGGAGGAGTTAGATTGTAGGG | ACGTTGGATGAGGGCAAGACTCCATCTCAA |
| 11235431 | ACGTTGGATGTCTGGCCACCAGAGAATGAC | ACGTTGGATGTACAGCAATGAGCCTTGGTC |
| 6592458 | ACGTTGGATGGGAGAATTGCTTGAATCCTGG | ACGTTGGATGTTGTTACCCAGGGTGTAGTG |
| 4945434 | ACGTTGGATGGAAGAAATCTGAGGCTCAGG | ACGTTGGATGATAGGGACAAGGTAGACAAC |
| 4945435 | ACGTTGGATGAAGAATCACATGAACCCGGG | ACGTTGGATGTTCAAGATGGAGTCTCGCTC |
| 11307657 | ACGTTGGATGGCCGTGTGCTTTTCCTCATG | ACGTTGGATGTGAGGTCGTGCCATTGTACT |
| 5792573 | ACGTTGGATGGCCGTGTGCTTTTCCTCATG | ACGTTGGATGTGAGGTCGTGCCATTGTACT |
| 1894004 | ACGTTGGATGTGAGGATCCGAGAGCTTTAC | ACGTTGGATGCATCCCTCCTAGTACAAGAC |
| 12276164 | ACGTTGGATGTTACCATGGCCTCATTGTCC | ACGTTGGATGGAAGCCTCAGACTTACCAAC |
| 4378421 | ACGTTGGATGTTTTTGAGACAGGGTCTTGC | ACGTTGGATGAGAAGTTTGAGGTTGCAGCG |
| 7116495 | ACGTTGGATGCAAGTAGCTGCTGCTACAAC | ACGTTGGATGTAGCGAGACTTCATCGCTAC |
| 7101701 | ACGTTGGATGATGAAGTCTCGCTATGTTGC | ACGTTGGATGACACTTTGGGAGGTCAATGC |
| 10736786 | ACGTTGGATGCGCTCCTTTAAACCAGTACC | ACGTTGGATGTGGGGAGCTAAAGAGAACAC |
| 11608165 | ACGTTGGATGTTGGAGACTTGTTCCCTCTG | ACGTTGGATGTTGCACTGCATTCTGTAAAG |
| 645603 | ACGTTGGATGTACATCTTCAGTACAGATGC | ACGTTGGATGCCACATGTGTGGATTCAACC |
| 661290 | ACGTTGGATGGTCTCTGCCTTGCATAGATC | ACGTTGGATGAACAGGAATCCCCAGAGATC |
| 7122209 | ACGTTGGATGAGCTGTGGAATCTCAAGCAG | ACGTTGGATGGAAAGACAGTGACACCAATG |
| 12417471 | ACGTTGGATGCTGAAGTCTCAGCTACTCAG | ACGTTGGATGAATCGTAGCTCACTGAAGCC |
| 541228 | ACGTTGGATGTAATCCCAGCACTTTGGGAG | ACGTTGGATGTTTTACTATGTTGGCCAGGC |
| 12273666 | ACGTTGGATGATTACTGGCATGAGCCATCG | ACGTTGGATGGATTATCAGTTTGGCTTGCC |
| 2511074 | ACGTTGGATGTCCAGCCTAAGAAACAGAGC | ACGTTGGATGACCGCACCCAGCTTAGTTTT |
| 3018311 | ACGTTGGATGAAAACAAACTAAGCTGGGTG | ACGTTGGATGAGAGACAGGGTTTCACTATG |
| 3018289 | ACGTTGGATGAGAGACAGGGTTTCACTATG | ACGTTGGATGAAAACAAACTAAGCTGGGTG |
| 679926 | ACGTTGGATGACTTGCCAGGATTAGGAGTG | ACGTTGGATGAGCTGCTTCCAACTCAACTG |
| 567026 | ACGTTGGATGATGTGAGCACAGCTAGCTAC | ACGTTGGATGTTGTTTGGAGAGCTCCTGGG |
| 564294 | ACGTTGGATGATATGTTCTGCAACTTGCCC | ACGTTGGATGTGCTGGTATGGAAACATGTC |
| 678193 | ACGTTGGATGTGCTGGTATGGAAACATGTC | ACGTTGGATGATATGTTCTGCAACTTGCCC |
| 677279 | ACGTTGGATGCTCCTGGTAGCTGGGACTAA | ACGTTGGATGGAACAGTTTGGGCAACATGG |
| 560777 | ACGTTGGATGGTGGCACATTCTTACATAGG | ACGTTGGATGCAAAGTGCTGGGATTACAGG |
| 676721 | ACGTTGGATGTCCAGATGTAGCAAAATGGG | ACGTTGGATGTGGCTGGCAATTCCTTAAAC |
| 7106529 | ACGTTGGATGATTTGGGAATATCTGTTGAC | ACGTTGGATGCAAGCTAATATTTCTCTAGG |
| 11602304 | ACGTTGGATGATTGCAGGCATGAGCCACTG | ACGTTGGATGCAGAGTGAGACTCCATGTCG |
| 585228 | ACGTTGGATGATTTGGGAGAGAATGCGAGG | ACGTTGGATGGTGGAGGAACTCAGTGTAAC |
| 578957 | ACGTTGGATGTCTTTTTAATTTCTCCTCGC | ACGTTGGATGTTCGTGGAGAGTATGATAGC |
| 7110215 | ACGTTGGATGGCTATCATACTCTCCACGAA | ACGTTGGATGGGTTTCACCATGTTGGCCAG |
| 3133233 | ACGTTGGATGAGGTGAAAGGATTGCTTGAG | ACGTTGGATGCACCACCATGCCTAGCTAAT |
| 3133230 | ACGTTGGATGTCAAGTGATCCTCTCACATC | ACGTTGGATGCAACATAGTGAGACCTCATC |
| 12576024 | ACGTTGGATGCAGCTTATTATGGCTTCAGC | ACGTTGGATGTATAGTCCCAGCTACTCAGG |
| 674319 | ACGTTGGATGAAATAGCTGGGCATGATGGC | ACGTTGGATGATCTTCCCGCTTTAGCTTCC |
| 675185 | ACGTTGGATGAATGACATGGGGAAATACTC | ACGTTGGATGCACCCAGCCTATATCCTGC |
| 5792574 | ACGTTGGATGTTCAAAAATGGGGCCATACC | ACGTTGGATGAGAGTGAGACCCTGTCTCAA |
| 12275272 | ACGTTGGATGGTACTGTAAGAACCAGGACC | ACGTTGGATGGCCCGGTCTTGTCTATTCTT |
| 10400327 | ACGTTGGATGCTGCCACTTTAAGAGGACTG | ACGTTGGATGTCTTCTTTCCCTCATTGCCC |
| 612255 | ACGTTGGATGGTTTGACTACTGGACTTGGG | ACGTTGGATGTCTGATCACCCAGCTGTGAC |
| 7101643 | ACGTTGGATGGATTCTGTGACTAAGTTACTG | ACGTTGGATGTAGCATCTCTTAACTCTTTC |
| 575871 | ACGTTGGATGAGCCTGGTCAAAAGATTCTG | ACGTTGGATGTAGCATCTCTTAACTCTTTC |
| 547208 | ACGTTGGATGGATAATGGAGAAAACTGGCC | ACGTTGGATGAGTCTGGGTGACAGAGAGAG |
| 2511075 | ACGTTGGATGTTGAAGATTTCTTCCAGGGC | ACGTTGGATGGGGCAAGACATGTACAATATC |
| 642573 | ACGTTGGATGAGATCCTGCTCAGGACAAAG | ACGTTGGATGTCCACCAGGAGGTAAGTCAA |
| 482197 | ACGTTGGATGGAAAATGAGAGTGGTGTGGG | ACGTTGGATGCTGCCCTGTTAGGATTTCAG |
| 656640 | ACGTTGGATGTGGGACCCAGGCACACTCCA | ACGTTGGATGCTTGAGCTCAGGAGTTCCAG |
| 671681 | ACGTTGGATGGAAATACTTGCTGAAATAGGG | ACGTTGGATGACATTTTGCCAAATTTGCTG |
| 541022 | ACGTTGGATGACTACCTAAGGAAACTGGGC | ACGTTGGATGCTACTATACCCCTCATGAGC |
| 951586 | ACGTTGGATGAACCAAGGTAAAGGGTTGGC | ACGTTGGATGCTACAGCCATCAGATACCTC |
| 2511076 | ACGTTGGATGCAAAGTGCTGGGATTACAGG | ACGTTGGATGTTTCTAGACAGCTCCTCCTC |
| 11235432 | ACGTTGGATGAGTAGCTGGGACTACAGGCA | ACGTTGGATGAGGAGATAGAGACCACGGTG |
| 3018308 | ACGTTGGATGAGTAGCTGGGACTACAGGCA | ACGTTGGATGAGGAGATAGAGACCACGGTG |
| 11606798 | ACGTTGGATGATAATGGCCTCTCCTCTACC | ACGTTGGATGAGCGAGACCCTGTCTCAAAA |
| 1791544 | ACGTTGGATGATTACAGGCCTGATCCACC | ACGTTGGATGTAGAACTTAGCTCACAGAGC |
| 3018304 | ACGTTGGATGCCTCCTGGGTTCAAGCGATT | ACGTTGGATGAGACCAGACTGGCCAACATG |
| 10751193 | ACGTTGGATGTCTCTCTATCACCCAGACTG | ACGTTGGATGTTGAACCTGGGAGGTGGAAG |
| 4945470 | ACGTTGGATGAATCACTTGAGCCCGAGAGG | ACGTTGGATGATGGAGTCTCACTCTGTCG |
| 3018291 | ACGTTGGATGAGATGGAGTCTCACTCTGTC | ACGTTGGATGACAGGAGAATCACTTGAGCC |
| 2511120 | ACGTTGGATGGGTAGATTTGGAGCTGAGAG | ACGTTGGATGATATGCCTAGAAGTGCTCAG |
| 671132 | ACGTTGGATGAACAGAAGTTGGTTCTTCCC | ACGTTGGATGGCCTTACTTAGCTGATGCTG |
| 4945475 | ACGTTGGATGTTATTCTCCAGGCTGGAGTC | ACGTTGGATGAGGCAGGAGAATCGCTTGAA |
| 3018292 | ACGTTGGATGAGCATGAGAAATCTTTTGGG | ACGTTGGATGCTACACCTGTAAACAGCAGC |
| 642618 | ACGTTGGATGTCTCAGATCTCATGCGTAAC | ACGTTGGATGCCAGAAGTTAACACAGAAAG |
| 552966 | ACGTTGGATGCCCAATCATTACCTCTTTACC | ACGTTGGATGATGTAGACGACAGAATGAGG |
| 6592459 | ACGTTGGATGGGAGAATGGTGTGAACCTGG | ACGTTGGATGAGATGGAGTCTCACTCTGTT |
| 607446 | ACGTTGGATGGATCATTCCCAAAGTCAATCC | ACGTTGGATGCATGCCCAGCTACAAAATAAT |
| 607070 | ACGTTGGATGCATACCAAAAGGTGTTGTCC | ACGTTGGATGAGAGATGGATTGACTTTGGG |
| 10713307 | ACGTTGGATGTAAAATACCAGATGGCAGGC | ACGTTGGATGAACTCTTGACCTCGTGATCC |
| 3018302 | ACGTTGGATGTTTGGGTTTATGGGTTGGGC | ACGTTGGATGACTGACCAGTCCAAGACAAC |
| 3750909 | ACGTTGGATGGTTGTATCTTGTTCGCCGTG | ACGTTGGATGGCACTCACAGAACGACTAAG |
| 3018301 | ACGTTGGATGACTAAGAACCTTCCTGCTCG | ACGTTGGATGTCTGTCCCATGTGAGTGTTG |
| 2511114 | ACGTTGGATGAACACTCACATGGGACAGAC | ACGTTGGATGGGGTGGTTCTTAAGCATCCC |
| 12270166 | ACGTTGGATGTGAGGTAGAAGGATCGCTTG | ACGTTGGATGTGGAGTGTAGTTGCGTGATC |
| 12270241 | ACGTTGGATGTTCTGAGGGCGTTTCTGATC | ACGTTGGATGCCCCAACGCAAACATAAAGC |
| 11606587 | ACGTTGGATGCGCTACTCAAAGCTCTACAG | ACGTTGGATGGAAAGCACTGGGTAGGTTAC |
| 686340 | ACGTTGGATGGTCTCATTGTCTTAGTCTCTC | ACGTTGGATGGCTGGAATGAATTTCAGATCC |
| 548961 | ACGTTGGATGAAAAAAAAATTGGGGGTGGG | ACGTTGGATGTATTTGCGCACTGCAACCAG |
| 549032 | ACGTTGGATGTACTGTTCACTCTCTACCCC | ACGTTGGATGCAAGAGCGAAACTCTGTCTC |
| 575831 | ACGTTGGATGATTACAGCAACCTCATGGCC | ACGTTGGATGATAGTGCCGGCAAGTAGTAG |
| 575878 | ACGTTGGATGATAGTGCCGGCAAGTAGTAG | ACGTTGGATGATTACAGCAACCTCATGGCC |
| 577435 | ACGTTGGATGGATTGCCATGGTAGCACTCG | ACGTTGGATGCCCTGCTGGTGTTTATGGAC |
| 579320 | ACGTTGGATGAAGATTCAACCTGGCTCCTG | ACGTTGGATGTGCATTTGGCTAGACTGCAG |
| 495567 | ACGTTGGATGTTCTGTCAACTTGGGATTGG | ACGTTGGATGATTGTTGTAGACTATTACCC |
| 636946 | ACGTTGGATGAGACCAGCCTAAGCAACATG | ACGTTGGATGTTAGCTTCCTGAGTAGCTGG |
| 493065 | ACGTTGGATGTGATCAGCTGGAGGGTTATG | ACGTTGGATGATGGCTTCAGCTGGTATCTC |
| 597513 | ACGTTGGATGCATTTCTTCTGGTCAGGCAC | ACGTTGGATGAGTCCACACCTTGTTCACAG |
| 598835 | ACGTTGGATGTTTTGTGTAGCACCAGCTCC | ACGTTGGATGTTTAACCAGCTTCTTGGGGC |
| 10683614 | ACGTTGGATGTTAGGAACTGCACTGGGTTG | ACGTTGGATGTTGGTACCCCAGAGTTATGC |
| 610004 | ACGTTGGATGAGAAGCTCATCCCACTTCAG | ACGTTGGATGGGTACCAACCCAGTATTTAC |
| 610041 | ACGTTGGATGCTGTTGTAGGTGCTGAAGTG | ACGTTGGATGCTGGCTGTCTACTACAGAAC |
| 673478 | ACGTTGGATGTAATACAAAGGTGGCAGCAG | ACGTTGGATGTTGACAAGGATAAGGACAAG |
| 670802 | ACGTTGGATGGAGACTGTCTTGATACCCAC | ACGTTGGATGCAGGCCCTCAAGGATTTGTT |
| 505041 | ACGTTGGATGATAGCCCCCTGTTTCCTTTC | ACGTTGGATGGGAGAGTGAGAGGTTTTTTTC |
| 2511116 | ACGTTGGATGGGGACAGAGTTGCCTAAAAC | ACGTTGGATGCTGTGGCAGCCTGGATATAC |
| 628025 | ACGTTGGATGTTCCTAATCCTGCTTCCCTC | ACGTTGGATGCTGCTACCTTATCACATGGG |
| 517837 | ACGTTGGATGGAATTCCTAATCCTGCTTCC | ACGTTGGATGCTGTGTCATTCTGTTTCAGG |
| 615000 | ACGTTGGATGTGCTTCAGTGGAATGGCAAG | ACGTTGGATGAAACCAGCCTCCATTCACTC |
| 482013 | ACGTTGGATGGCCTTGGAGGAGCTTATATG | ACGTTGGATGCTCTTCTGTGCTGCTACAAC |
| 693391 | ACGTTGGATGCACTTTGATATCCTGTCTCTC | ACGTTGGATGATCACTGTATATCCAGTACC |
| 2511079 | ACGTTGGATGAGCTGAGTAGATGATAGCAC | ACGTTGGATGGGAAAGAGAGACAGGATATC |
| 2250866 | ACGTTGGATGCCCCCTAAAGCCAAAAAAGC | ACGTTGGATGTCCTTGGCTCCTCATTTACC |
| 2508860 | ACGTTGGATGAAGTGCTGGGATTACAGGCG | ACGTTGGATGCTCCCTGTCGGAAAAATGGG |
| 7483267 | ACGTTGGATGGAAACCCCGCCTCTACTAAA | ACGTTGGATGAAGTGATTCTCCTGCCTCAG |
| 2511078 | ACGTTGGATGGAAACCCCGCCTCTACTAAA | ACGTTGGATGAAGTGATTCTCCTGCCTCAG |
| 2508859 | ACGTTGGATGAGCCTCTGATGTACCAGTTG | ACGTTGGATGTGGCAACAGAGTGAGACTCC |
| 2508858 | ACGTTGGATGTACCTCTTCCACAGGAGAAC | ACGTTGGATGACAACTGGTACATCAGAGGC |
| 11235435 | ACGTTGGATGAGCATGATGCTCTGTCTTCC | ACGTTGGATGCCCTTCACTGAAGACCTTTC |
| 11235436 | ACGTTGGATGGTCATACAGTATGGCTGTGG | ACGTTGGATGTGGGCAGAGAAGGAAGAATG |
| 639435 | ACGTTGGATGGACCCTAGTAGTCCTAAAGG | ACGTTGGATGCTTCTCATCTGCTCAACAGC |
| 12285624 | ACGTTGGATGTTTTTGAGACGGAGTCTCGC | ACGTTGGATGTGAACCTGGAAGGCGGAGTTT |
| NUMA1-AD | ACGTTGGATGCCTGGCTAACATGGTGAAAC | ACGTTGGATGGGACTATAGGCACCTACTAC |
| 624363 | ACGTTGGATGTTATCTAACTCTCTGGGCCC | ACGTTGGATGCCTGGTACTCTGTGAAGAGT |
| NUMA1-AE | ACGTTGGATGAGGCAGCTTTGATTCTTGGG | ACGTTGGATGTGGTCTGCTTTCTCTCTCCA |
| 1053573 | ACGTTGGATGTATCGAGGGTGACAAACCAA | ACGTTGGATGGTCTGGATGAGCTTGAATTTG |
| 1053511 | ACGTTGGATGGACCTAGGGAGATCCCCTTC | ACGTTGGATGTGAGCACCAAGTCAGGGAGA |
| 1063863 | ACGTTGGATGTGAGGCCAACAGAGAGGGTG | ACGTTGGATGTGGAGATACCAGTTTGGGTG |
| 12137 | ACGTTGGATGTGAAGACATACCAGGCTCTG | ACGTTGGATGGGTAGAACCTTTTGGGACTG |
| 4365081 | ACGTTGGATGTTCTACCCTCACTTGCTCAG | ACGTTGGATGACAGTACCCTAACCTGCTAC |
| 4466868 | ACGTTGGATGACTGCGGCTTATACAGTACC | ACGTTGGATGTTCTAGCCTTCCTCTTCTCC |
| 3750911 | ACGTTGGATGGGATCCCAAACTGTACAACC | ACGTTGGATGGGATAGCTGCTTATGCCTAC |
| 510925 | ACGTTGGATGGATGAGGCTTACCTGGGCTG | ACGTTGGATGGGGTGCTGACCAAGAGAGAG |
| 595062 | ACGTTGGATGTCATGTCAGCCCTGTTTACC | ACGTTGGATGAGACTCAATCTAGGCCCATC |
| 1053443 | ACGTTGGATGTCTCTTCCATCCTTGCCAAG | ACGTTGGATGTGTACTCATGCTGCTCCATG |
| 542752 | ACGTTGGATGCCTTTAGATCCCAGTGCTTG | ACGTTGGATGCTAGGACCTGGCAAAGAAAG |
| 3897579 | ACGTTGGATGCAAGAACAGCCTTCCTCATC | ACGTTGGATGTAGGGCTTTGTCTGGACTCG |
| 11235437 | ACGTTGGATGAACAGCGCCATTGTTGTTTG | ACGTTGGATGTATGAACCCAGGGTTTGAGG |
| 2508856 | ACGTTGGATGTTGGTGAGTCAGCATTAGCC | ACGTTGGATGCATAAAGAGCAAGAGTGCCC |
| 5792575 | ACGTTGGATGATGGAGCCAGCTTTCCTTAG | ACGTTGGATGCACCCTTCCTCCCTAAATAC |
| 659513 | ACGTTGGATGCCATGTACTTTATCCATGGG | ACGTTGGATGCTTAGCACAGACATAGCAGC |
| 10898820 | ACGTTGGATGAACCCACACTAATATGACGG | ACGTTGGATGCGTTACAGTTTACAAAGGGC |
| 2276397 | ACGTTGGATGTGGGTGAGTGAGCAGCTAG | ACGTTGGATGGATTTCTGGAGTAAGGCTGG |
| 3750908 | ACGTTGGATGGCTTTCCTGCTGTCTTCCAC | ACGTTGGATGAAGGGTCAAGTGAATGTGTT |
| 3793938 | ACGTTGGATGAAAAGCAGGACCAGGCATCG | ACGTTGGATGTAGGTCATACAGTGTGTCGG |
| 602285 | ACGTTGGATGAACCCTTAAAGAGCTCCAGC | ACGTTGGATGGTGACACCAATGAATGACGG |
| 2276396 | ACGTTGGATGTTAGGGAAGGAAGTGTGCCG | ACGTTGGATGTAAACGAGACCGTAGTGCTG |
| 2276395 | ACGTTGGATGACAGTCCAGGGAAGCCAAAC | ACGTTGGATGGTCAAAACTCAACAAGCAACC |
| 1806778 | ACGTTGGATGAGCTTGCAGTGATCCGAGAT | ACGTTGGATGGTGTTGTTCATGGTCAATGG |
| 4073394 | ACGTTGGATGTCCTTCTTCTTTACTGGGTC | ACGTTGGATGTCCTTGCACCAGAGCTAATC |
| 471547 | ACGTTGGATGCAGGGCCTTACCTCCATTCT | ACGTTGGATGTCCTAGACAAAGCCTTGTGC |
| 606136 | ACGTTGGATGAGGAGTGAGACTCTAGACAG | ACGTTGGATGAGCTTCCCTGGGCATCTAAA |
| 605241 | ACGTTGGATGACTGGAGAAATCCAAGAGGG | ACGTTGGATGCTCCACCAACAGCGCTGTAG |
| 686063 | ACGTTGGATGCATCACTTGACCCTAGGTGC | ACGTTGGATGTCTTGGGAAGTCACTCTAGG |
| 685749 | ACGTTGGATGCATCACTTGACCCTAGGTGC | ACGTTGGATGTCTTGGGAAGTCACTCTAGG |
| 533207 | ACGTTGGATGACAAGGCAGGTAAGACAAGC | ACGTTGGATGCTCAGGTTTTCTGAGTCCTG |
| 476753 | ACGTTGGATGTCCCTGGGCATCTAAACCTC | ACGTTGGATGAGGAGTGAGACTCTAGACAG |
| NUMA1-AF | ACGTTGGATGCAGGATGCAGAAAACCTGAG | ACGTTGGATGCATCACTTGACCCTAGGTGC |

TABLE 29

| dbSNP rs# | Extend Primer | Term Mix |
|---|---|---|
| 4945392 | AAATCTCTTTTTCTGTTGAGT | ACT |
| 7938496 | CTGCACTCCAGCCTGGG | ACG |
| 7926550 | GAACTCCTGAGCTCAAA | ACT |
| 7945374 | AGCTCTATTCTCCAGGG | CGT |
| 7949480 | TCCTGACCACATGATCC | ACT |
| 7102523 | CCACACCCATCTGTGTC | ACT |
| 7121260 | AATTAAGTGGTGTAAGATCAAT | ACT |
| 7131230 | CACCGGAGTAATTTTCAG | ACG |
| 7128317 | TTAGTCCTTGCCTCCCC | ACG |
| 2276385 | GGCAAACTATGACCAAATC | ACT |
| 2276384 | TTGGTCATAGTTTGCCC | ACT |
| 2276383 | AGAAGGCCCAAAGGCTA | ACT |
| 1892921 | CCTAACAGATAGTTTTTCAAC | ACG |
| 1892920 | CACTTTTGTAATTCTGGCA | ACG |
| 7942626 | TGCGTTTCTCAGATTGG | ACG |
| 7124429 | TCAAATTGAAAACAGTACATTG | ACT |
| 7114081 | TTTGGAATTAGACAGTGAAA | CGT |
| 7125718 | GTAACTCATTACCCCTTC | ACG |
| 1055452 | TCCTGAGTGGCCCAAGC | ACT |
| 3829215 | GCCACTCAGGACCCCTC | ACG |
| 1541306 | AGTTGTCCCAAAACAAAT | ACG |
| 3814722 | CCATATTCCCTAGAAACC | CGT |
| 1939240 | CTCCTAGGGATGTCAGC | CGT |
| 5743655 | GGAAGGCAGGTGGTGGC | ACT |
| 5743656 | GCTTCTCTTCCCCAGCT | ACT |
| 5743657 | GTCCAAAGCTCTTTTCAT | ACT |
| 3814721 | ACCCCAGGCAGCCTTGC | ACG |
| 5743658 | CATCCTAGGTTCCTCAC | CGT |
| 5743659 | CCCTTCCCTCCTGTTAG | ACT |
| 5743660 | CAGGCTACCCACTCCCC | ACG |
| 5743661 | TCCCAGGCTACCCACTC | CGT |
| 5743662 | GGACCACTTCAGGCTCA | ACT |
| 2298455 | GATACCAGACTGGGATC | ACT |
| 5743664 | TAGACCTCACCCTACCC | ACG |
| 5743665 | CCTTCACTCCAAGGCAA | ACT |
| 5743667 | GAGATGTCTGGGTTCTAG | ACG |
| 5743668 | CAGCGGCTCTGGAGACA | ACT |
| 5743669 | CCTTGGTTGCAGTGGAC | ACG |
| 1573503 | TGGGAGCCCAGGTGACA | ACT |
| 5743670 | ACGTCCGTTACAGAAGT | ACT |
| 5743671 | CCTAGAGAACTGCTCTG | ACG |
| 1892919 | TGCTCTGCTGTGCCCTC | ACG |
| 5743672 | TGCTGAAGTTGGGGAAG | ACG |
| 2735786 | CAGGTGGGTGGGAAGGA | ACT |
| 5743673 | CTGTGCTCCCACGTTCC | ACG |
| 5743674 | ATTAGTTCACAGGCTGC | CGT |
| 1062452 | TGTAGGCTGCGTGGATG | CGT |
| 5743675 | AGAGAGAATGGCAGGAG | ACT |
| 5743676 | CCATTAGCCTTCCTAAC | ACT |
| 760246 | GAACGCTCAAGCCTGGT | ACG |
| 11345794 | CAGGGGAACACAGGCGCT | ACT |
| 5743677 | AAAGAAAAGAGAGAACAGC | ACG |
| 5743678 | TTCCCACACCGATCAAG | ACT |
| 14537 | CCGGGGATGGATCTTTT | ACG |
| 3168177 | CCCACCCCAGTGCAAGT | ACG |
| 5743679 | CCTTCAGCAGCAGCTCA | ACG |
| 1541304 | ACCCCACTCCTGAGACA | ACG |
| 1053725 | GATCTAGAAGGGACCATAA | ACT |
| 5743680 | CAAACCCCTAGAGACAG | ACG |
| 949324 | GGCGGAGGACCAGGTGA | ACT |
| 949323 | CGGAGGACCAGGTGAGG | ACT |
| 5743681 | TGGGGCTCACAAGGGAG | ACT |
| 5743682 | ATAACCCTGGGGCTCAC | ACT |
| 5743683 | ATTCTCATGGAGAGCAC | ACG |
| 5743684 | GCCACCACCACAGCCAG | ACG |
| 5743685 | GTTGAGGATGCTGAAGG | ACG |
| 2845857 | GCGGGACCTTGCTGATA | ACT |
| 2852365 | TGCTGTGCCACGCTCTA | ACG |
| 11538641 | CCCACGCCCCATGACTCC | CGT |
| 11538639 | GGCCACCAGCTGTTTCCCA | ACG |
| 11235417 | GGACAGTTAGGCAGACAGTA | ACT |
| 11538644 | AAACGGGTCTCCCTAGAGC | CGT |
| 11538643 | GACCCCCAAGAGACCCTG | ACG |
| 2845858 | CGAGTGTAGAGTTCTGG | ACT |
| 2155146 | GAGCTCCTGCCTACCCC | CGT |
| 2852364 | CAGGTGGGATTCCAAGG | ACT |
| 2845859 | CCATAAACTTGAAGCCC | ACT |
| 2845860 | CAAGCCTTGGCAGGTGA | ACT |
| 11538642 | CTGGGAAGCAGGAGCAGAC | CGT |
| 7949430 | GTCTTCCAGAAGCTAGAT | ACT |
| 7938674 | AGTGCCAGGGACTGTAC | ACT |
| 2735787 | ACAAAGGCAAGGGCCTG | ACG |
| 11538640 | CACCTATCTCCCAGCGCC | ACG |
| 10736784 | CACCAAGCTTCCTGAACAGC | CGT |
| 10736785 | TGGAGTGGGGTGTGGTTAG | ACT |
| 11826059 | AAGAATTTGAAACTGAAGAGTAC | ACT |
| 2503 | AGGCCCATTGGAGACCG | ACT |
| 2155145 | TGGTATAATACCCGTTTTTC | ACT |
| 4477459 | GATGTGGACTGAAGGGC | ACT |
| NUMA1-AA | CCAAGTGCCTTAGAGTA | ACT |
| 5019605 | ACCCAGGTGCAGAGCCTTA | ACT |
| 5019604 | CATGCCAAGTGCCTTAG | CGT |
| 5019603 | AGGTGCAGAGCCTTACT | ACT |
| 3793941 | AACACTGGTGACACTCT | ACG |
| 11235418 | GGCTTCCTAGGAGGGCTAC | ACT |
| 3793940 | GCTCTTCCTCTTTGTAG | ACT |
| 2032353 | TGGGCAAGTGTCCTAAG | CGT |
| 3934448 | TAAGCCCACAATCCTCC | CGT |
| 7951267 | ACCCCTTGTCCCTGATA | ACT |
| 1053603 | CCAACTCTTGGACATACTT | ACG |
| 1053602 | ACAGGCCCCAAGCTGGA | ACT |
| 11235419 | CGGGAGGCAGAGCAGATG | ACT |
| 1053601 | TAAGCCGGCAGCAGAAC | ACT |
| 3750913 | CTCAGCTGTGTGCTGGGCA | ACT |
| 3750912 | CTTGGGGAGGCCCATCA | ACT |
| 1053600 | GCTGGAGGCTCGATTAC | CGT |
| 1057992 | GTGAGTCCACACCAACC | CGT |
| 2298789 | GAGGAAGGGTATACGGT | ACT |
| 10898813 | CTATGCTGGGTTCATCCCTAA | CGT |
| 11278712 | GAATCCCCATCACCCAGAG | ACG |
| NUMA1-AB | CAGTGAGAAGAGAGTATGGG | ACG |
| 949325 | TCCTCTTCCCTATGCCG | ACG |
| NUMA1-AC | GCCTGTTGCTGATTGCTTCA | ACT |
| 949326 | GAATACCCTTCTTCCTC | ACT |
| 2298456 | GCATAAAGGTGCAAATCTTT | CGT |
| 7949845 | GATGTCAGGAGTTCGAGACC | ACT |
| 7949989 | CCAGCTACTCGGGAGGCT | ACT |
| 1573500 | CCCAAGTAGCTAGAACTA | ACT |
| 4945411 | GAAGTGAAAGTTCTGTGGGG | ACT |
| 3831387 | GGGAAAAAAAAACACACACACA | ACT |
| 11235422 | GCCAAACAGGAGGCAGTCA | CGT |
| 10128658 | GGCTGTCTCTCTTCTCAGTG | ACG |
| 2298457 | AGTTCACCCTGTTTTCT | ACT |
| 3838779 | CCGAACTTCAAGGAGACAGAA | ACT |
| 1573501 | TCATCTGTGCTGCCTCC | ACT |
| 11235424 | CGGAGGCAGCACAGATGAA | ACG |
| 10898814 | GTTGAAGGCTGAGATGGCAAA | ACT |
| 7930142 | CTGAGAACCTCTTTTTATTTTA | ACT |
| 7930544 | CCATTTACAACTGCCCA | ACG |
| 7930721 | TTTGCCAGAAAACTTAGAG | ACT |
| 7930722 | GACAAAAATGTCAGACCT | ACT |
| 6592456 | GGCCAAGTGGCCAAGCA | ACT |
| 11235425 | GTAGGCGATATTGAAATCCCA | ACT |
| 6592457 | CACTGTAGGTTTATAACCTG | ACT |
| 10898815 | TACCAGTTCCTAACTGCCAC | ACT |
| 11235426 | TTGCCCCAGGCCAAGGATA | ACT |
| 10898816 | CTTGGCCTGGGGCAAACAAT | ACG |
| 1939247 | GGGCTCTCTGATGGTTGCAT | CGT |
| 1939246 | GGACACTATCATAGCTCAT | ACT |
| 1939245 | AAGTTGTTTTTAAATGTGAAAGT | ACT |
| 1939244 | GGCCCATGCCTGTAATC | CGT |
| 1939243 | GAGTAGCAGGGACTACG | ACG |
| 4245463 | CAAGACCCTATCTCAAAAAG | CGT |
| 4944258 | GGCCTGAAGTTTTCAGA | CGT |
| 7926751 | TCATGCATGTTCTTTGTTT | ACT |
| 12282917 | ACGTGGTGAGACCCTGTCT | ACG |
| 12282918 | CAACGTGGTGAGACCCTGT | ACG |
| 7937582 | CCTGGGCAACGTGGTGA | ACT |
| 10898817 | AGATGGAGTCTTGCTCTGTC | ACT |
| 7480015 | GAGGCTGAGGCAGGAGGAT | ACG |
| 7123992 | CATTAAGGTCAGCATTTACCAC | ACG |
| 1573502 | AGACAAGCAACATAACCTCTTT | ACG |
| 7127865 | AGAACACCAGGACAGGTGC | ACT |
| 4945426 | GAAGGTGCTCACTGTCTAGTA | CGT |
| 11235428 | GTCTGGCTGTTGCCGCCC | ACT |
| 7122489 | GCTACAATCGCAAGGATCTG | ACT |
| 1894003 | AAACCACCTGCTCAGAC | ACT |
| 1548348 | CTCCTGGCCTATCCGTCC | ACG |
| 1892923 | TGGGGCGCGTACACATACA | ACG |
| 5792570 | AACCCTTTGCTCTGAAAGGC | ACT |
| 7115200 | TGCCTGGCAGGAGCGATC | ACT |
| 4945430 | AAGCAGAGTCTACAAGTCCC | ACT |
| 11235429 | GTCTGACTCTTAGAAGACAAAA | ACG |
| 1939242 | CCACACCTCTCTGTTTC | ACG |
| 9666346 | GTGAGCCGTCGTGCCAAG | ACT |
| 7101553 | GGGATTACAGGTGTGAGCC | ACT |
| 7124000 | GCAACATGGCGAAACCCTG | ACG |
| 12291664 | CAACATAGTGAGAGCCTGTCT | ACG |
| 12291778 | ATAGGCTAAAATGTTTTACATATAA | ACT |
| 12291781 | TAACCAGTAAAACTTCATGATTTA | ACT |
| 12291787 | CTCCTGAGTAGCTGGGACT | ACG |
| 12291788 | GCCTCCTGAGTAGCTGGGA | ACG |
| 12291833 | CAGGAGGCTAGGGTGGGA | ACT |
| 10793016 | TAGGTCAGTAGAATAGTGTCG | ACG |
| 5792571 | CTAACTCCTCCCCCCCCC | CGT |
| 12293529 | AATCTAACTCCTCCCCCCC | ACT |
| 4338555 | GGAACATTCACTTGGGGACTT | ACG |
| 11235431 | TGGTGGGTGGTTGGGATGA | ACT |
| 6592458 | GTTGTGGTGAGCCGAGATC | ACT |
| 4945434 | AGGCTCAGGAATTAGGTGAC | ACG |
| 4945435 | CCGGGAGGCAGAGGTTGC | ACT |
| 11307657 | TCCTCATGAAAATCAACTTTTTTT | CGT |
| 5792573 | TCCTCATGAAAATCAACTTTTTTT | CGT |
| 1894004 | GATCCGAGAGCTTTACAAACACT | ACT |
| 12276164 | CCTCATTGTCCCTCTCCAGA | ACT |
| 4378421 | GGCTGGAGTGCAGTGGCA | ACG |
| 7116495 | TGCTGCTACAACAGGCACG | ACG |
| 7101701 | TGTTGCCTAGGCTGGTCTC | ACT |
| 10736786 | CAGTACCTACTCTTATTTTCTC | ACT |
| 11608165 | CCCTCTGCTTCCCCCTGG | ACT |
| 645603 | CAGATGCTTTTTTTTCCC | ACG |
| 661290 | CATAGATCTAGTGGGCATC | ACT |
| 7122209 | GAAAATGATGATCCCCAAATCT | ACT |
| 12417471 | CTCAGCTACTCAGGAGGCT | ACT |
| 541228 | GGCAGGCGGATCACAAGGT | ACT |
| 12273666 | CATGAGCCATCGCGCCTG | ACT |
| 2511074 | AAGAAACAGAGCGAGACACC | ACT |
| 3018311 | CAGGTGGATCGCCTGAGG | ACT |
| 3018289 | TGTTGGCCAGGCTGTTCTC | ACT |
| 679926 | CCAGGATTAGGAGTGTGACAA | ACT |
| 567026 | CCTTGTCACACTCCTAA | ACG |
| 564294 | TCTGCAACTTGCCCTTTTCA | ACG |
| 678193 | ATGGAAACATGTCAAATAAATTT | ACT |
| 677279 | GTAGCTGGGACTAAAGGTGC | ACT |
| 560777 | TAAGGAATTGCCAGCCATGA | ACG |
| 676721 | CACATTCTTACATAGGATTTTTT | ACG |
| 7106529 | AATACACAAGCTTTTCAACCCA | CGT |
| 11602304 | GAGCCACTGTGCCCAGAC | ACG |
| 585228 | GACCCTATTACTTAAAATGAC | ACT |
| 578957 | TCTCCTCGCATTCTCTCCC | CGT |
| 7110215 | ACCTGAGGTCAGGAGTTTGA | ACT |
| 3133233 | CAGCCTGGGCAACATAGTGA | ACT |
| 3133230 | GGTGTGCACCACCATGCCT | ACT |
| 12576024 | TGGCTTCAGCCTCCCAGG | CGT |
| 674319 | TGGCATGCGCCTGTAGT | ACG |
| 675185 | ATGGGGAAATACTCAAGAAA | ACT |
| 5792574 | ATAATAGTTTTATCCTGCCTTTTT | ACT |
| 12275272 | CACCTTCTCTCAACCTAACCT | ACG |
| 10400327 | ACTTTAAGAGGACTGGCAAGA | ACG |
| 612255 | GGAGTGGAATTTCTTTTTTAC | ACG |
| 7101643 | GCAATTCTCTTTCTTACTCTAAA | ACG |
| 575871 | GCAATTCTCTTTCTTACTCTA | ACT |
| 547208 | GCCTTTCTTTTCTTTTTTTTT | ACG |
| 2511075 | CCAGGGCCAAAATTTATCATTAT | ACT |
| 642573 | CTCAGGACAAAGCTGACCTAT | ACT |
| 482197 | GGGCCTCCTGAGGAAGAAG | ACG |
| 656640 | GGCACACTCCACCACACCT | ACT |
| 671681 | AAAGATTCAGAAAATGAAGGA | ACG |
| 541022 | AACTGGGCATTTGGAGGTCA | ACT |
| 951586 | GGGGCCATTTGGCAGAA | ACT |
| 2511076 | ATTACAGGCGTGAGCCACCG | ACG |
| 11235432 | CCCGCCACCGCGCCCAG | ACT |
| 3018308 | CACCCGCCACCGCGCCC | ACT |
| 11606798 | CTACCTCCTACTGTGGGCAA | ACT |
| 1791544 | CTGATCCACCGCAGCCAG | ACG |
| 3018304 | TTACAGGCATGCGCCACCA | CGT |
| 10751193 | CCAGACTGGAGTACAGTGG | ACT |
| 4945470 | GTTGCAGTGAGCCAAGACTG | ACG |
| 3018291 | CTGTCGCCCAGGCTGGAG | ACT |
| 2511120 | TTAATAGCTGGCACAGTCTA | ACT |
| 671132 | GTTGGTTCTTCCCAGTTGC | ACG |
| 4945475 | TCCAGTGGTGCAATCTCGG | ACT |
| 3018292 | GAGAGAAATCCTCTGTATCT | ACT |
| 642618 | ATGTTGAACTTCCTCATTCTGT | ACG |
| 552966 | TCTCAGATCTCATGCGTAAC | ACT |
| 6592459 | GTGTGAACCTGGGAGGCG | ACT |
| 607446 | TCCCAAAGTCAATCCATCTCTAAG | ACG |
| 607070 | GTCCAGACTCAAAATGA | ACT |
| 10713307 | TGGCTCACGCCTGTAATCC | CGT |
| 3018302 | TGGGGACGGGGAAGGTGCT | ACT |
| 3750909 | TGGGGGAAATCTCAGATCCC | ACT |
| 3018301 | CCCTCATCACCTTTCAC | ACT |
| 2511114 | GGGATAGGCGTTGAGATATA | ACG |
| 12270166 | CCCGGAGATTGAAGCTGCA | ACT |
| 12270241 | TCTGATCTCCCAGGTTATGC | ACG |
| 11606587 | TCATTTTTTTTAGTACTCACGAC | ACT |
| 686340 | CTAGTTCCTTTTCCCCATTAAAA | ACT |
| 548961 | GTGGGGTAGAGAGTGAA | ACG |
| 549032 | CTCTCTACCCCACCCCC | CGT |
| 575831 | GCCCTCTCCTTTGTAGT | ACT |
| 575878 | GGTGCTTAACAAACACATTAA | CGT |
| 577435 | ATGGTAGCACTCGTAACACT | ACT |
| 579320 | GGCTCCTGTCCACCCCC | CGT |
| 495567 | AGTGAATAACATTAAAGTACGATA | ACG |
| 636946 | GCCTAAGCAACATGGCAAAAC | ACG |
| 493065 | GATGTGTAGGTGAGAGACATGC | ACT |
| 597513 | AGGCACAGAGCAGCAAGCAC | CGT |
| 598835 | CTCCTGTTGCATTTTCTGAATG | ACT |
| 10683614 | CTGCACTGGGTTGCTCTGT | ACG |
| 610004 | ATCCCACTTCAGCACCTACA | ACT |
| 610041 | TGCTGAAGTGGGATGAGCTTCT | ACT |
| 673478 | AAGGGGAGGTCGACTGGG | ACT |
| 670802 | GTCTTGATACCCACCCTCTG | ACT |
| 505041 | ATCGGGGCAAAATCAAA | CGT |
| 2511116 | GGCTGGAGCTGCTCTTA | ACG |
| 628025 | TCCCTCCCCATACCTCCT | ACT |
| 517837 | AATCCTGCTTCCCTCCCCA | ACG |
| 615000 | GGAGGCAGAAGCCAGAT | ACT |
| 482013 | AAGATGGGAGCAAGCTGGCAA | ACG |
| 693391 | GATATCCTGTCTCTCTTTCCAT | ACT |
| 2511079 | GCCTATAGAATTCACTTCA | ACT |
| 2250866 | TAAAGCCAAAAAAGCAAAGAT | ACT |
| 2508860 | CGTGAGCCGCTGTGCCC | ACT |
| 7483267 | AAAATTAGCTGGGCGTGGTG | ACT |
| 2511078 | AATTAGCTGGGCGTGGTGG | ACG |
| 2508859 | GTACCAGTTGTTTGTTGG | ACT |
| 2508858 | GTGCGCAGTGACAGTTA | ACT |
| 11235435 | GCTTGCTGTGATAGAATCGTA | ACT |
| 11235436 | GTATGGCTGTGGAGGGTTG | ACT |
| 639435 | CCTAAGCATAGACAGCATGG | ACT |
| 12285624 | GACGGAGTCTCGCTCTGTC | ACT |
| NUMA1-AD | TGAAACCCCGTCTCTACTAAA | CGT |
| 624363 | GGGCCCCTTTGTACTTTTCA | ACT |
| NUMA1-AE | GGCCCTGCCTCAGACTCA | ACT |
| 1053573 | ACCAAACATGTAAAAACCC | CGT |
| 1053511 | GATCCCCTTCCCACCCT | ACG |
| 1063863 | GGATCTCCCTAGGTCCC | ACG |
| 12137 | TCCTTTTGGCCCCCACCC | ACG |
| 4365081 | CTCACTTGCTCAGGCTTCTA | ACT |
| 4466868 | ATACAGTACCCTAACCTGCTA | ACT |
| 3750911 | ACCAGCTCCTCTTTTGC | ACT |
| 510925 | CTGGGCAGGGCAGACTGT | ACT |
| 595062 | CTGTGACACTCTTGAGGGC | ACT |
| 1053443 | ATGTGTCTGCTGCAGACT | ACG |
| 542752 | TGCTATGTCTCAGCTCC | CGT |
| 3897579 | TGTTCGCTTCCAAGATG | ACT |
| 11235437 | AGTACAGTGGCACAGTCCTA | ACT |
| 2508856 | GGATGTCCCCCTGGGCC | ACG |
| 5792575 | TAGTTTTATCTTTGGCAAACATTT | ACT |
| 659513 | CCCTAGACCAGGTGGCCAGTCA | ACG |
| 10898820 | CACACTAATATGACGGATGTCT | ACT |
| 2276397 | GCTAGCACAGCACAGAGACC | ACG |
| 3750908 | GCTGTCTTCCACCCTATCCC | ACG |
| 3793938 | ATCGATTATTTTCTCCTGCT | ACG |
| 602285 | CTCGAGACTAAGCCCCACCC | ACT |
| 2276396 | GCCTACCTATCTGCCCC | ACT |
| 2276395 | AGACATGCAGTAGACTTAGT | ACT |
| 1806778 | CGACAGAGCCAGACTCC | ACT |
| 4073394 | TTACTGGGTCAGCTCCCTCC | ACT |
| 471547 | TGGTTAGACCCTCTGTT | CGT |
| 606136 | TGTCCTCCTACCCCATGGCA | ACT |
| 605241 | AGCAAGCTGGTGTCAGA | ACT |
| 686063 | AGCCCAGAAATTTTATGCAATTGA | ACG |
| 685749 | CATGATTTCTCAGGTTTTCTGA | ACT |
| 533207 | AAGACAAGCAGAAATGGTGGAG | ACT |
| 476753 | TCTAAACCTCAGTTGCC | ACT |
| NUMA1-AF | GAAATCATGAGTCAATTGCATAA | CGT |

### Genetic Analysis of Allelotyping Results

Allelotyping results are shown for cases and controls in Table 30. The allele frequency for the A2 allele is noted in the fifth and sixth columns for breast cancer pools and control pools, respectively, where “AF” is allele frequency. SNPs with blank allele frequencies were untyped.

TABLE 30

| | | | | | | | | Breast |
|---|---|---|---|---|---|---|---|---|
| | | | | | | | | Cancer |
| | Position in | Chromosome | A1/A2 | | | | | Associated |
| dbSNP rs# | SEQ ID NO: 4 | Position | Allele | Case AF | Control AF | p-Value | OR | Allele |
| 4945392 | 203 | 71420953 | T/C | T = 0.999 | T = 0.998 | 0.936 | 0.74 | T |
| | | | | C = 0.001 | C = 0.002 | | | |
| 7938496 | 1236 | 71421986 | G/A | G = 0.988 | G = 0.966 | 0.233 | 0.34 | G |
| | | | | A = 0.012 | A = 0.034 | | | |
| 7926550 | 1768 | 71422518 | C/G | C = 1 | C = 1 | 0.956 | 0.00 | C |
| | | | | G = 0 | G = 0.000 | | | |
| 7945374 | 2101 | 71422851 | A/T | A = 1 | A = 0.999 | 0.964 | 0.72 | A |
| | | | | T = 0.000 | T = 0.001 | | | |
| 7949480 | 3146 | 71423896 | T/C | T = 0.126 | T = 0 | 0.0000166 | 0.00 | T |
| | | | | C = 0.874 | C = 1 | | | |
| 7102523 | 4476 | 71425226 | T/C | T = 0.046 | T = 0.031 | 0.337 | 0.67 | T |
| | | | | C = 0.954 | C = 0.969 | | | |
| 7121260 | 6124 | 71426874 | A/G | A = 0.954 | A = 0.994 | 0.00306 | 6.99 | G |
| | | | | G = 0.046 | G = 0.006 | | | |
| 7131230 | 6719 | 71427469 | C/T | C = 0.319 | C = 0.368 | 0.106 | 1.24 | T |
| | | | | T = 0.681 | T = 0.632 | | | |
| 7128317 | 6925 | 71427675 | G/A | G = 0.906 | G = 0.958 | 0.00239 | 2.33 | A |
| | | | | A = 0.094 | A = 0.042 | | | |
| 2276385 | 7060 | 71427810 | C/G | C = 0.962 | C = 0.996 | 0.00315 | 9.57 | G |
| | | | | G = 0.038 | G = 0.004 | | | |
| 2276384 | 7081 | 71427831 | T/C | T = 0.996 | T = 0.994 | 0.864 | 0.74 | T |
| | | | | C = 0.004 | C = 0.006 | | | |
| 2276383 | 7090 | 71427840 | A/G | A = 0.0660 | A = 0.015 | 0.00194 | 0.21 | A |
| | | | | G = 0.934 | G = 0.985 | | | |
| 1892921 | 7696 | 71428446 | C/T | C = 0.091 | C = 0.036 | 0.0015 | 0.37 | C |
| | | | | T = 0.909 | T = 0.964 | | | |
| 1892920 | 7989 | 71428739 | C/T | C = 0.997 | C = 0.997 | 0.973 | 1.09 | T |
| | | | | T = 0.003 | T = 0.003 | | | |
| 7942626 | 9407 | 71430157 | G/A | G = 0.061 | G = 0.026 | 0.0929 | 0.41 | G |
| | | | | A = 0.939 | A = 0.974 | | | |
| 7124429 | 9699 | 71430449 | T/G | T = 0.001 | T = 0.001 | 0.956 | 0.25 | T |
| | | | | G = 0.999 | G = 0.999 | | | |
| 7114081 | 9905 | 71430655 | A/T | A = 0.001 | A = 0.001 | 0.952 | 0.53 | A |
| | | | | T = 0.999 | T = 0.999 | | | |
| 7125718 | 9984 | 71430734 | C/T | C = 1 | C = 1 | 0.944 | 0.39 | C |
| | | | | T = 0.000 | T = 0.000 | | | |
| 1055452 | 13023 | 71433773 | T/C | T = 0.106 | T = 0.081 | 0.211 | 0.75 | T |
| | | | | C = 0.894 | C = 0.919 | | | |
| 3829215 | 13047 | 71433797 | G/A | G = 0.968 | G = 0.984 | 0.22 | 1.95 | A |
| | | | | A = 0.032 | A = 0.016 | | | |
| 1541306 | 13571 | 71434321 | C/T | C = 0.2 | C = 0.131 | 0.00398 | 0.60 | C |
| | | | | T = 0.800 | T = 0.869 | | | |
| 3814722 | 14086 | 71434836 | C/A | C = 0.999 | C = 0.998 | 0.953 | 0.79 | C |
| | | | | A = 0.001 | A = 0.002 | | | |
| 1939240 | 14337 | 71435087 | T/A | T = 0.001 | T = 0.002 | 0.886 | 22.72 | A |
| | | | | A = 0.999 | A = 0.998 | | | |
| 5743655 | 14462 | 71435212 | T/C | T = 0.007 | T = | | | |
| | | | | C = 0.993 | C = | | | |
| 5743656 | 14518 | 71435268 | A/G | A = 0.003 | A = 0.001 | 0.819 | 0.29 | A |
| | | | | G = 0.997 | G = 0.999 | | | |
| 5743657 | 14695 | 71435445 | A/G | A = 0.002 | A = 0.002 | 0.943 | 0.70 | A |
| | | | | G = 0.998 | G = 0.998 | | | |
| 3814721 | 14830 | 71435580 | G/A | G = 0.152 | G = 0.087 | 0.00211 | 0.53 | G |
| | | | | A = 0.848 | A = 0.913 | | | |
| 5743658 | 15282 | 71436032 | C/A | C = 0.038 | C = 0.021 | 0.386 | 0.54 | C |
| | | | | A = 0.962 | A = 0.979 | | | |
| 5743659 | 15436 | 71436186 | A/G | A = 0.004 | A = 0.003 | 0.892 | 0.67 | A |
| | | | | G = 0.996 | G = 0.997 | | | |
| 5743660 | 15745 | 71436495 | G/A | G = 1 | G = 1 | 0.967 | 0.62 | G |
| | | | | A = 0.000 | A = 0.000 | | | |
| 5743661 | 15748 | 71436498 | G/T | G = 1 | G = 1 | 0.981 | 0.40 | G |
| | | | | T = 0.000 | T = 0.000 | | | |
| 5743662 | 15797 | 71436547 | T/C | T = 1 | T = 1 | 0.998 | 0.96 | T |
| | | | | C = 0.000 | C = 0.000 | | | |
| 2298455 | 16036 | 71436786 | T/G | T = 0.908 | T = 0.919 | 0.57 | 1.15 | G |
| | | | | G = 0.092 | G = 0.081 | | | |
| 5743664 | 16646 | 71437396 | G/A | G = 1 | G = 0.999 | 0.947 | 0.56 | G |
| | | | | A = 0.000 | A = 0.001 | | | |
| 5743665 | 16713 | 71437463 | A/G | A = 1 | A = 1 | 0.971 | 2.04 | G |
| | | | | G = 0.000 | G = 0.000 | | | |
| 5743667 | 16859 | 71437609 | G/A | G = 0.999 | G = 0.998 | 0.924 | 0.64 | G |
| | | | | A = 0.001 | A = 0.002 | | | |
| 5743668 | 16914 | 71437664 | G/— | G = 0.002 | G = 0.002 | 0.949 | 0.69 | G |
| | | | | — = 0.998 | — = 0.998 | | | |
| 5743669 | 17422 | 71438172 | C/T | C = 1 | C = 0.999 | 0.91 | 0.40 | C |
| | | | | T = 0.000 | T = 0.001 | | | |
| 1573503 | 17499 | 71438249 | A/G | A = 0.044 | A = 0.019 | 0.0808 | 0.42 | A |
| | | | | G = 0.956 | G = 0.981 | | | |
| 5743670 | 17646 | 71438396 | G/C | G = 0.005 | G = 0.004 | 0.935 | 0.84 | G |
| | | | | C = 0.995 | C = 0.996 | | | |
| 5743671 | 17681 | 71438431 | G/A | G = 0.999 | G = 0.999 | 0.939 | 1.51 | A |
| | | | | A = 0.001 | A = 0.001 | | | |
| 1892919 | 17733 | 71438483 | C/T | C = 0.085 | C = 0.046 | 0.0269 | 0.52 | C |
| | | | | T = 0.915 | T = 0.954 | | | |
| 5743672 | 17841 | 71438591 | G/A | G = 0.999 | G = 0.999 | 0.934 | 0.64 | G |
| | | | | A = 0.001 | A = 0.001 | | | |
| 2735786 | 17963 | 71438713 | A/G | A = 0.004 | A = 0.004 | 0.935 | 1.23 | G |
| | | | | G = 0.996 | G = 0.996 | | | |
| 5743673 | 18011 | 71438761 | G/A | G = 1 | G = 0.999 | 0.907 | 0.46 | G |
| | | | | A = 0.000 | A = 0.001 | | | |
| 5743674 | 18238 | 71438988 | A/T | A = 1 | A = 1 | 0.921 | 6.24 | T |
| | | | | T = 0.000 | T = 0.000 | | | |
| 1062452 | 18569 | 71439319 | C/A | C = 1 | C = 0.998 | 0.862 | 0.41 | C |
| | | | | A = 0.000 | A = 0.002 | | | |
| 5743675 | 18672 | 71439422 | T/C | T = 0.003 | T = 0.002 | 0.957 | 0.79 | T |
| | | | | C = 0.997 | C = 0.998 | | | |
| 5743676 | 18715 | 71439465 | A/G | A = 0.003 | A = 0.003 | 0.954 | 1.22 | G |
| | | | | G = 0.997 | G = 0.997 | | | |
| 760246 | 18927 | 71439677 | C/T | C = 0.001 | C = 0.001 | 0.995 | 0.92 | C |
| | | | | T = 0.999 | T = 0.999 | | | |
| 11345794 | 19266 | 71440016 | T/— | T = 1 | T = 0.999 | 0.861 | 0.00 | T |
| | | | | — = 0 | — = 0.001 | | | |
| 5743677 | 19290 | 71440040 | C/T | C = 1 | C = 1 | 0.996 | 0.96 | C |
| | | | | T = 0.000 | T = 0.000 | | | |
| 5743678 | 19456 | 71440206 | T/G | T = 0.999 | T = 0.999 | 0.995 | 1.05 | G |
| | | | | G = 0.001 | G = 0.001 | | | |
| 14537 | 19511 | 71440261 | C/T | C = 0.999 | C = 1 | 0.926 | 1.86 | T |
| | | | | T = 0.001 | T = 0.000 | | | |
| 3168177 | 19575 | 71440325 | G/A | G = 1 | G = 1 | 0.964 | 0.59 | G |
| | | | | A = 0.000 | A = 0.000 | | | |
| 5743679 | 19591 | 71440341 | G/A | G = 1 | G = 0.999 | 0.905 | 0.42 | G |
| | | | | A = 0.000 | A = 0.001 | | | |
| 1541304 | 19636 | 71440386 | G/A | G = 0.994 | G = 0.994 | 0.969 | 0.92 | G |
| | | | | A = 0.006 | A = 0.006 | | | |
| 1053725 | 19770 | 71440520 | A/G | A = 0.002 | A = 0.001 | 0.867 | 0.02 | A |
| | | | | G = 0.998 | G = 0.999 | | | |
| 5743680 | 20002 | 71440752 | G/A | G = 0.001 | G = 0.002 | 0.941 | 1.82 | A |
| | | | | A = 0.999 | A = 0.998 | | | |
| 949324 | 20084 | 71440834 | G/C | G = 0.998 | G = 0.996 | 0.821 | 0.57 | G |
| | | | | C = 0.002 | C = 0.004 | | | |
| 949323 | 20086 | 71440836 | A/C | A = 0.573 | A = 0.555 | 0.613 | 0.93 | A |
| | | | | C = 0.427 | C = 0.445 | | | |
| 5743681 | 20246 | 71440996 | T/C | T = 0.005 | T = 0.005 | 0.982 | 0.92 | T |
| | | | | C = 0.995 | C = 0.995 | | | |
| 5743682 | 20253 | 71441003 | T/C | T = 0.999 | T = 0.999 | 0.987 | 0.92 | T |
| | | | | C = 0.001 | C = 0.001 | | | |
| 5743683 | 20300 | 71441050 | G/A | G = 0.999 | G = 1 | 0.933 | 1.90 | A |
| | | | | A = 0.001 | A = 0.000 | | | |
| 5743684 | 20539 | 71441289 | G/A | G = 1 | G = 1 | 0.985 | 1.59 | A |
| | | | | A = 0.000 | A = 0.000 | | | |
| 5743685 | 20682 | 71441432 | C/T | C = 0.999 | C = 0.998 | 0.927 | 0.67 | C |
| | | | | T = 0.001 | T = 0.002 | | | |
| 2845857 | 20773 | 71441523 | G/C | G = 0.603 | G = 0.632 | 0.403 | 1.13 | C |
| | | | | C = 0.397 | C = 0.368 | | | |
| 2852365 | 20776 | 71441526 | G/A | G = 0.717 | G = 0.584 | 0.0000557 | 0.55 | G |
| | | | | A = 0.283 | A = 0.416 | | | |
| 11538641 | 20904 | 71441654 | C/A | C = 1 | C = 1 | 0.988 | | A |
| | | | | A = 0.000 | A = 0 | | | |
| 11538639 | 20918 | 71441668 | C/T | C = 1 | C = 0.999 | 0.866 | 0.12 | C |
| | | | | T = 0.000 | T = 0.001 | | | |
| 11235417 | 20978 | 71441728 | A/G | A = 0.001 | A = 0.002 | 0.942 | 1.83 | G |
| | | | | G = 0.999 | G = 0.998 | | | |
| 11538644 | 21269 | 71442019 | C/A | C = 0.992 | C = 0.999 | 0.709 | 5.16 | A |
| | | | | A = 0.008 | A = 0.001 | | | |
| 11538643 | 21354 | 71442104 | C/T | C = 1 | C = 0.999 | 0.86 | 0.10 | C |
| | | | | T = 0.000 | T = 0.001 | | | |
| 2845858 | 21488 | 71442238 | T/C | T = 0.002 | T = 0.001 | 0.969 | 0.74 | T |
| | | | | C = 0.998 | C = 0.999 | | | |
| 2155146 | 21656 | 71442406 | G/T | G = 0.985 | G = 0.988 | 0.843 | 1.25 | T |
| | | | | T = 0.015 | T = 0.012 | | | |
| 2852364 | 21793 | 71442543 | T/C | T = 0.001 | T = 0.001 | 0.957 | 0.50 | T |
| | | | | C = 0.999 | C = 0.999 | | | |
| 2845859 | 22084 | 71442834 | A/G | A = 0.999 | A = 0.999 | 0.991 | 1.06 | G |
| | | | | G = 0.001 | G = 0.001 | | | |
| 2845860 | 22219 | 71442969 | T/C | T = 0.998 | T = 0.996 | 0.789 | 0.51 | T |
| | | | | C = 0.002 | C = 0.004 | | | |
| 11538642 | 22815 | 71443565 | G/T | G = 1 | G = 1 | 0.986 | 2.89 | T |
| | | | | T = 0.000 | T = 0.000 | | | |
| 7949430 | 22858 | 71443608 | T/C | T = 0.003 | T = 0.001 | 0.767 | 0.20 | T |
| | | | | C = 0.997 | C = 0.999 | | | |
| 7938674 | 22895 | 71443645 | T/C | T = 1 | T = 0.999 | 0.982 | 0.83 | T |
| | | | | C = 0.000 | C = 0.001 | | | |
| 2735787 | 23431 | 71444181 | C/T | C = 0.976 | C = 0.998 | 0.323 | 10.97 | T |
| | | | | T = 0.024 | T = 0.002 | | | |
| 11538640 | 23968 | 71444718 | C/T | C = 0.001 | C = 0.002 | 0.903 | 3.80 | T |
| | | | | T = 0.999 | T = 0.998 | | | |
| 10736784 | 24469 | 71445219 | T/A | T = 1 | T = 0.992 | 0.685 | 0.11 | T |
| | | | | A = 0.000 | A = 0.008 | | | |
| 10736785 | 24470 | 71445220 | A/C | A = 0.995 | A = 0.998 | 0.744 | 2.15 | C |
| | | | | C = 0.005 | C = 0.002 | | | |
| 11826059 | 24853 | 71445603 | T/C | T = 1 | T = 1 | 1 | | |
| | | | | C = 0 | C = 0 | | | |
| 2503 | 24961 | 71445711 | T/C | T = 0.071 | T = 0.041 | 0.0658 | 0.55 | T |
| | | | | C = 0.929 | C = 0.959 | | | |
| 2155145 | 25177 | 71445927 | A/C | A = 0.994 | A = 0.995 | 0.894 | 1.36 | C |
| | | | | C = 0.006 | C = 0.005 | | | |
| 4477459 | 26174 | 71446924 | T/C | T = 0.047 | T = 0.022 | 0.112 | 0.46 | T |
| | | | | C = 0.953 | C = 0.978 | | | |
| NUMA1-AA | 26334 | 71447084 | T/— | T = 0.993 | T = | | | |
| | | | | — = 0.007 | — = | | | |
| 5019605 | 26337 | 71447087 | C/G | C = 0.996 | C = 0.992 | 0.855 | 0.49 | C |
| | | | | G = 0.004 | G = 0.008 | | | |
| 5019604 | 26338 | 71447088 | A/T | A = 0.001 | A= | | | |
| | | | | T = 0.999 | T = | | | |
| 5019603 | 26339 | 71447089 | C/G | C = 0.993 | C = 0.993 | 0.979 | 0.93 | C |
| | | | | G = 0.007 | G = 0.007 | | | |
| 3793941 | 26965 | 71447715 | G/A | G = 0.551 | G = 0.565 | 0.645 | 1.06 | A |
| | | | | A = 0.449 | A = 0.435 | | | |
| 11235418 | 27278 | 71448028 | C/G | C = 1 | C = 1 | 0.98 | | G |
| | | | | G = 0.000 | G = 0 | | | |
| 3793940 | 28042 | 71448792 | A/G | A = 0.003 | A = 0.002 | 0.92 | 0.62 | A |
| | | | | G = 0.997 | G = 0.998 | | | |
| 2032353 | 28520 | 71449270 | G/T | G = 1 | G = 0.999 | 0.937 | 0.53 | G |
| | | | | T = 0.000 | T = 0.001 | | | |
| 3934448 | 28533 | 71449283 | G/T | G = 1 | G = 1 | 0.976 | 0.60 | G |
| | | | | T = 0.000 | T = 0.000 | | | |
| 7951267 | 28764 | 71449514 | A/G | A = 0.003 | A = 0.002 | 0.945 | 0.75 | A |
| | | | | G = 0.997 | G = 0.998 | | | |
| 1053603 | 29685 | 71450435 | G/A | G = 0.999 | G = 0.999 | 0.977 | 0.84 | G |
| | | | | A = 0.001 | A = 0.001 | | | |
| 1053602 | 30738 | 71451488 | A/G | A = 0.003 | A = 0.002 | 0.974 | 0.87 | A |
| | | | | G = 0.997 | G = 0.998 | | | |
| 11235419 | 31175 | 71451925 | T/C | T = 0.002 | T = 0.002 | 0.957 | 1.28 | C |
| | | | | C = 0.998 | C = 0.998 | | | |
| 1053601 | 31490 | 71452240 | A/G | A = 0.002 | A = 0.002 | 0.987 | 1.12 | G |
| | | | | G = 0.998 | G = 0.998 | | | |
| 3750913 | 31726 | 71452476 | G/C | G = 0.114 | G = 0.037 | 0.00136 | 0.29 | G |
| | | | | C = 0.886 | C = 0.963 | | | |
| 3750912 | 31770 | 71452520 | A/G | A = 0.006 | A = 0.003 | 0.825 | 0.49 | A |
| | | | | G = 0.994 | G = 0.997 | | | |
| 1053600 | 31792 | 71452542 | T/A | T = 0.001 | T = 0.001 | 0.932 | 2.89 | A |
| | | | | A = 0.999 | A = 0.999 | | | |
| 1057992 | 32980 | 71453730 | C/A | C = 0.982 | C = 0.997 | 0.38 | 5.78 | A |
| | | | | A = 0.018 | A = 0.003 | | | |
| 2298789 | 33398 | 71454148 | A/G | A = 0.998 | A = 1 | 0.833 | 3.32 | G |
| | | | | G = 0.002 | G = 0.000 | | | |
| 10898813 | 33580 | 71454330 | T/A | T = 0.075 | T = 0.049 | 0.174 | 0.63 | T |
| | | | | A = 0.925 | A = 0.951 | | | |
| NUMA1-AB | 34941 | 71455691 | C/T | C = 0.098 | C = 0.009 | 0.00000449 | 0.08 | C |
| | | | | T = 0.902 | T = 0.991 | | | |
| 949325 | 35214 | 71455964 | G/A | G = 0.875 | G = 0.932 | 0.0108 | 1.96 | A |
| | | | | A = 0.125 | A = 0.068 | | | |
| NUMA1-AC | 35629 | 71456379 | G/C | G = 0.061 | G = 0.007 | 0.000244 | 0.10 | G |
| | | | | C = 0.939 | C = 0.993 | | | |
| 949326 | 35864 | 71456614 | T/C | T = 0.975 | T = 0.989 | 0.233 | 2.28 | C |
| | | | | C = 0.025 | C = 0.011 | | | |
| 2298456 | 36273 | 71457023 | A/T | A = 0.89 | A = 0.996 | 0.0000541 | 28.64 | T |
| | | | | T = 0.110 | T = 0.004 | | | |
| 7949845 | 37281 | 71458031 | A/G | A = 0.998 | A = 0.998 | 0.995 | 1.04 | G |
| | | | | G = 0.002 | G = 0.002 | | | |
| 7949989 | 37378 | 71458128 | A/G | A = 0.036 | A = 0.03 | 0.764 | 0.83 | A |
| | | | | G = 0.964 | G = 0.970 | | | |
| 1573500 | 38348 | 71459098 | C/G | C = 0.172 | C = 0.116 | 0.0235 | 0.63 | C |
| | | | | G = 0.828 | G = 0.884 | | | |
| 4945411 | 38608 | 71459358 | A/G | A = 0.938 | A = 0.984 | 0.00931 | 4.04 | G |
| | | | | G = 0.062 | G = 0.016 | | | |
| 11235422 | 39049 | 71459799 | C/A | C = 1 | C = 1 | 0.958 | 2.79 | A |
| | | | | A = 0.000 | A = 0.000 | | | |
| 10128658 | 39077 | 71459827 | C/T | C = 0.168 | C = 0.102 | 0.0027 | 0.56 | C |
| | | | | T = 0.832 | T = 0.898 | | | |
| 2298457 | 39769 | 71460519 | T/C | T = 0.192 | T = 0.089 | 0.00000854 | 0.41 | T |
| | | | | C = 0.808 | C = 0.911 | | | |
| 3838779 | 40555 | 71461305 | T/— | T = 0.071 | T = 0.029 | 0.0118 | 0.38 | T |
| | | | | — = 0.929 | — = 0.971 | | | |
| 1573501 | 41251 | 71462001 | T/C | T = 0.996 | T = 0.996 | 0.925 | 1.20 | C |
| | | | | C = 0.004 | C = 0.004 | | | |
| 11235424 | 41270 | 71462020 | C/T | C = 1 | C = 1 | 0.965 | 9.18 | T |
| | | | | T = 0.000 | T = 0.000 | | | |
| 10898814 | 42063 | 71462813 | T/C | T = 0.193 | T = 0.108 | 0.000353 | 0.50 | T |
| | | | | C = 0.807 | C = 0.892 | | | |
| 7930142 | 42574 | 71463324 | T/C | T = 0.945 | T = 0.996 | 0.0024 | 12.18 | C |
| | | | | C = 0.055 | C = 0.004 | | | |
| 7930544 | 42912 | 71463662 | C/T | C = 0.149 | C = 0.082 | 0.00224 | 0.50 | C |
| | | | | T = 0.851 | T = 0.918 | | | |
| 7930721 | 43039 | 71463789 | A/C | A = 0.759 | A = 0.806 | 0.198 | 1.31 | C |
| | | | | C = 0.241 | C = 0.194 | | | |
| 7930722 | 43042 | 71463792 | C/G | C = 0.504 | C = 0.419 | 0.0187 | 0.71 | C |
| | | | | G = 0.496 | G = 0.581 | | | |
| 6592456 | 43221 | 71463971 | A/G | A = 0.994 | A = 0.997 | 0.734 | 1.91 | G |
| | | | | G = 0.006 | G = 0.003 | | | |
| 11235425 | 43518 | 71464268 | T/C | T = 0.001 | T = 0.001 | 0.992 | 1.17 | C |
| | | | | C = 0.999 | C = 0.999 | | | |
| 6592457 | 43644 | 71464394 | G/C | G = 0.0640 | G = 0.041 | 0.216 | 0.61 | G |
| | | | | C = 0.936 | C = 0.959 | | | |
| 10898815 | 44981 | 71465731 | A/G | A = 0.578 | A = 0.577 | 0.98 | 1.00 | A |
| | | | | G = 0.422 | G = 0.423 | | | |
| 11235426 | 45013 | 71465763 | T/C | T = 0 | T = 0.002 | 0.884 | | C |
| | | | | C = 1 | C = 0.998 | | | |
| 10898816 | 45038 | 71465788 | C/T | C = 0.999 | C = 0.998 | 0.948 | 0.77 | C |
| | | | | T = 0.001 | T = 0.002 | | | |
| 1939247 | 46079 | 71466829 | T/A | T = 0.098 | T = 0.054 | 0.0313 | 0.52 | T |
| | | | | A = 0.902 | A = 0.946 | | | |
| 1939246 | 47848 | 71468598 | T/G | T = 1 | T = 0.999 | 0.946 | 0.65 | T |
| | | | | G = 0.000 | G = 0.001 | | | |
| 1939245 | 48090 | 71468840 | A/G | A = 0.984 | A = 0.998 | 0.254 | 6.60 | G |
| | | | | G = 0.016 | G = 0.002 | | | |
| 1939244 | 48147 | 71468897 | G/T | G = 0.664 | G = 0.635 | 0.347 | 0.88 | G |
| | | | | T = 0.336 | T = 0.365 | | | |
| 1939243 | 48293 | 71469043 | C/T | C = 0.073 | C = 0.005 | 0.00105 | 0.06 | C |
| | | | | T = 0.927 | T = 0.995 | | | |
| 4245463 | 48771 | 71469521 | C/A | C = 0.038 | C = 0.011 | 0.0369 | 0.28 | C |
| | | | | A = 0.962 | A = 0.989 | | | |
| 4944258 | 48951 | 71469701 | A/T | A = 0.943 | A = 0.994 | 0.00184 | 8.94 | T |
| | | | | T = 0.057 | T = 0.006 | | | |
| 7926751 | 49972 | 71470722 | T/G | T = 0.103 | T = 0.046 | 0.00205 | 0.42 | T |
| | | | | G = 0.897 | G = 0.954 | | | |
| 12282917 | 50161 | 71470911 | G/A | G = 0.999 | G = 0.998 | 0.871 | 0.46 | G |
| | | | | A = 0.001 | A = 0.002 | | | |
| 12282918 | 50163 | 71470913 | G/A | G = 0.999 | G = 0.996 | 0.792 | 0.46 | G |
| | | | | A = 0.001 | A = 0.004 | | | |
| 7937582 | 50171 | 71470921 | T/C | T = 0.992 | T = 0.996 | 0.645 | 2.05 | C |
| | | | | C = 0.008 | C = 0.004 | | | |
| 10898817 | 51289 | 71472039 | T/C | T = 0.957 | T = 0.976 | 0.383 | 1.80 | C |
| | | | | C = 0.043 | C = 0.024 | | | |
| 7480015 | 51699 | 71472449 | G/A | G = 0.848 | G = 0.841 | 0.771 | 0.95 | G |
| | | | | A = 0.152 | A = 0.159 | | | |
| 7123992 | 52005 | 71472755 | C/T | C = 0.083 | C = 0.049 | 0.0525 | 0.56 | C |
| | | | | T = 0.917 | T = 0.951 | | | |
| 1573502 | 52986 | 71473736 | G/A | G = 0.111 | G = 0.049 | 0.000664 | 0.40 | G |
| | | | | A = 0.889 | A = 0.951 | | | |
| 7127865 | 53339 | 71474089 | G/C | G = 0.146 | G = 0.077 | 0.00143 | 0.49 | G |
| | | | | C = 0.854 | C = 0.923 | | | |
| 4945426 | 53704 | 71474454 | C/A | C = 0.113 | C = 0.0640 | 0.0563 | 0.54 | C |
| | | | | A = 0.887 | A = 0.936 | | | |
| 11235428 | 54615 | 71475365 | G/C | G = 1 | G = 0.999 | 0.92 | 0.33 | G |
| | | | | C = 0.000 | C = 0.001 | | | |
| 7122489 | 55307 | 71476057 | A/C | A = 1 | A = 0.999 | 0.957 | 0.53 | A |
| | | | | C = 0.000 | C = 0.001 | | | |
| 1894003 | 55834 | 71476584 | T/C | T = 0.195 | T = 0.118 | 0.00112 | 0.55 | T |
| | | | | C = 0.805 | C = 0.882 | | | |
| 1548348 | 56475 | 71477225 | G/A | G = 0.977 | G = 0.985 | 0.51 | 1.52 | A |
| | | | | A = 0.023 | A = 0.015 | | | |
| 1892923 | 57053 | 71477803 | G/A | G = 0.022 | G = 0.007 | 0.444 | 0.28 | G |
| | | | | A = 0.978 | A = 0.993 | | | |
| 5792570 | 57355 | 71478105 | —/G | — = 0 | — = 0.001 | 0.962 | | G |
| | | | | G = 1 | G = 0.999 | | | |
| 7115200 | 57718 | 71478468 | T/G | T = 0.541 | T = 0.524 | 0.614 | 0.93 | T |
| | | | | G = 0.459 | G = 0.476 | | | |
| 4945430 | 58703 | 71479453 | G/C | G = 0.279 | G = 0.191 | 0.00233 | 0.61 | G |
| | | | | C = 0.721 | C = 0.809 | | | |
| 11235429 | 58772 | 71479522 | C/T | C = 0.001 | C = 0.001 | 0.945 | 2.60 | T |
| | | | | T = 0.999 | T = 0.999 | | | |
| 1939242 | 59140 | 71479890 | C/T | C = 0.097 | C = 0.058 | 0.0268 | 0.57 | C |
| | | | | T = 0.903 | T = 0.942 | | | |
| 9666346 | 59450 | 71480200 | G/C | G = 0.939 | G = 0.958 | 0.251 | 1.49 | C |
| | | | | C = 0.061 | C = 0.042 | | | |
| 7101553 | 59461 | 71480211 | T/C | T = 0.999 | T = 0.999 | 0.978 | 0.84 | T |
| | | | | C = 0.001 | C = 0.001 | | | |
| 7124000 | 59896 | 71480646 | C/T | C = 0.938 | C = 0.955 | 0.437 | 1.38 | T |
| | | | | T = 0.062 | T = 0.045 | | | |
| 12291664 | 60157 | 71480907 | G/A | G = 0.93 | G = 0.958 | 0.213 | 1.73 | A |
| | | | | A = 0.070 | A = 0.042 | | | |
| 12291778 | 60369 | 71481119 | A/G | A = 0 | A = 0 | 1 | | |
| | | | | G = 1 | G = 1 | | | |
| 12291781 | 60395 | 71481145 | A/G | A = 0.004 | A = 0.003 | 0.928 | 0.58 | A |
| | | | | G = 0.996 | G = 0.997 | | | |
| 12291787 | 60424 | 71481174 | G/A | G = 0.997 | G = 0.998 | 0.979 | 1.08 | A |
| | | | | A = 0.003 | A = 0.002 | | | |
| 12291788 | 60426 | 71481176 | G/A | G = 0.998 | G = 0.997 | 0.944 | 0.81 | G |
| | | | | A = 0.002 | A = 0.003 | | | |
| 12291833 | 60456 | 71481206 | A/G | A = 0.002 | A = 0.003 | 0.841 | 2.31 | G |
| | | | | G = 0.998 | G = 0.997 | | | |
| 10793016 | 60621 | 71481371 | G/A | G = 0.097 | G = 0.054 | 0.0219 | 0.53 | G |
| | | | | A = 0.903 | A = 0.946 | | | |
| 5792571 | 61207 | 71481957 | G/— | G = 0.202 | G = 0.155 | 0.0543 | 0.72 | G |
| | | | | — = 0.798 | — = 0.845 | | | |
| 12293529 | 61209 | 71481959 | G/C | G = 1 | G = 1 | 0.99 | 0.84 | G |
| | | | | C = 0.000 | C = 0.000 | | | |
| 4338555 | 61254 | 71482004 | C/T | C = 0.949 | C = 0.979 | 0.0733 | 2.50 | T |
| | | | | T = 0.051 | T = 0.021 | | | |
| 11235431 | 61854 | 71482604 | C/G | C = 0.003 | C = 0.004 | 0.972 | 1.10 | G |
| | | | | G = 0.997 | G = 0.996 | | | |
| 6592458 | 63103 | 71483853 | A/G | A = 0.986 | A = 0.995 | 0.601 | 2.43 | G |
| | | | | G = 0.014 | G = 0.005 | | | |
| 4945434 | 64645 | 71485395 | C/T | C = 0.14 | C = 0.083 | 0.0063 | 0.55 | C |
| | | | | T = 0.860 | T = 0.917 | | | |
| 4945435 | 64777 | 71485527 | T/C | T = 0.998 | T = 0.996 | 0.853 | 0.55 | T |
| | | | | C = 0.002 | C = 0.004 | | | |
| 11307657 | 64980 | 71485730 | T/— | T = 0.018 | T = 0.009 | 0.724 | 0.51 | T |
| | | | | — = 0.982 | — = 0.991 | | | |
| 5792573 | 64987 | 71485737 | T/— | T = 0.037 | T = 0.01 | 0.306 | 0.24 | T |
| | | | | — = 0.963 | — = 0.990 | | | |
| 1894004 | 65375 | 71486125 | T/C | T = 0.977 | T = 0.984 | 0.537 | 1.43 | C |
| | | | | C = 0.023 | C = 0.016 | | | |
| 12276164 | 65561 | 71486311 | A/G | A = 0.004 | A = 0.002 | 0.826 | 0.50 | A |
| | | | | G = 0.996 | G = 0.998 | | | |
| 4378421 | 67554 | 71488304 | C/T | C = 0.996 | C = 0.996 | 0.959 | 0.90 | C |
| | | | | T = 0.004 | T = 0.004 | | | |
| 7116495 | 67643 | 71488393 | C/T | C = 0 | C = | | | |
| | | | | T = 1 | T = | | | |
| 7101701 | 67713 | 71488463 | A/G | A = 0.483 | A = | | | |
| | | | | G = 0.517 | G = | | | |
| 10736786 | 68832 | 71489582 | T/C | T = 0.959 | T = 0.991 | 0.164 | 4.57 | C |
| | | | | C = 0.041 | C = 0.009 | | | |
| 11608165 | 70184 | 71490934 | T/G | T = 1 | T = 1 | 0.962 | 0.28 | T |
| | | | | G = 0.000 | G = 0.000 | | | |
| 645603 | 70415 | 71491165 | G/A | G = 0.967 | G = 0.97 | 0.829 | 1.10 | A |
| | | | | A = 0.033 | A = 0.030 | | | |
| 661290 | 71572 | 71492322 | A/G | A = 0.186 | A = 0.171 | 0.648 | 0.90 | A |
| | | | | G = 0.814 | G = 0.829 | | | |
| 7122209 | 71732 | 71492482 | T/C | T = 1 | T = 1 | 0.994 | 2.26 | C |
| | | | | C = 0.000 | C = 0.000 | | | |
| 12417471 | 72829 | 71493579 | A/G | A = 0.125 | A = 0.118 | 0.741 | 0.94 | A |
| | | | | G = 0.875 | G = 0.882 | | | |
| 541228 | 73154 | 71493904 | C/G | C = 0.994 | C = 0.996 | 0.935 | 1.29 | G |
| | | | | G = 0.006 | G = 0.004 | | | |
| 12273666 | 73221 | 71493971 | A/G | A = 0.058 | A = 0.047 | 0.511 | 0.81 | A |
| | | | | G = 0.942 | G = 0.953 | | | |
| 2511074 | 74343 | 71495093 | T/C | T = 0.946 | T = 0.975 | 0.176 | 2.19 | C |
| | | | | C = 0.054 | C = 0.025 | | | |
| 3018311 | 74462 | 71495212 | T/G | T = 0.969 | T = 0.993 | 0.151 | 4.57 | G |
| | | | | G = 0.031 | G = 0.007 | | | |
| 3018289 | 74471 | 71495221 | T/C | T = 0.086 | T = 0.069 | 0.375 | 0.78 | T |
| | | | | C = 0.914 | C = 0.931 | | | |
| 679926 | 75494 | 71496244 | A/G | A = 0.923 | A = 0.953 | 0.0831 | 1.70 | G |
| | | | | G = 0.077 | G = 0.047 | | | |
| 567026 | 75510 | 71496260 | G/A | G = 0.943 | G = 0.954 | 0.513 | 1.24 | A |
| | | | | A = 0.057 | A = 0.046 | | | |
| 564294 | 75827 | 71496577 | G/A | G = 1 | G = 1 | 0.988 | 1.14 | A |
| | | | | A = 0.000 | A = 0.000 | | | |
| 678193 | 75831 | 71496581 | T/G | T = 0.133 | T = 0.082 | 0.0189 | 0.58 | T |
| | | | | G = 0.867 | G = 0.918 | | | |
| 677279 | 76027 | 71496777 | T/C | T = 0.979 | T = 0.988 | 0.477 | 1.79 | C |
| | | | | C = 0.021 | C = 0.012 | | | |
| 560777 | 76160 | 71496910 | C/T | C = 0.925 | C = 0.96 | 0.0341 | 1.94 | T |
| | | | | T = 0.075 | T = 0.040 | | | |
| 676721 | 76196 | 71496946 | C/T | C = 0.105 | C = 0.053 | 0.00468 | 0.47 | C |
| | | | | T = 0.895 | T = 0.947 | | | |
| 7106529 | 76378 | 71497128 | A/T | A = 0.999 | A = 0.989 | 0.526 | 0.11 | A |
| | | | | T = 0.001 | T = 0.011 | | | |
| 11602304 | 77306 | 71498056 | G/A | G = 0.999 | G = 0.999 | 0.991 | 0.93 | G |
| | | | | A = 0.001 | A = 0.001 | | | |
| 585228 | 78847 | 71499597 | C/G | C = 0.158 | C = 0.088 | 0.00104 | 0.51 | C |
| | | | | G = 0.842 | G = 0.912 | | | |
| 578957 | 78918 | 71499668 | C/A | C = 0.003 | C = 0.003 | 0.967 | 0.88 | C |
| | | | | A = 0.997 | A = 0.997 | | | |
| 7110215 | 79117 | 71499867 | A/G | A = 0.243 | A = 0.25 | 0.826 | 1.04 | G |
| | | | | G = 0.757 | G = 0.750 | | | |
| 3133233 | 79811 | 71500561 | C/G | C = 0 | C = 0.003 | 0.842 | | G |
| | | | | G = 1 | G = 0.997 | | | |
| 3133230 | 79843 | 71500593 | T/C | T = 0.924 | T = 0.946 | 0.252 | 1.42 | C |
| | | | | C = 0.076 | C = 0.054 | | | |
| 12576024 | 80448 | 71501198 | G/T | G = 1 | G = 1 | 0.992 | 0.88 | G |
| | | | | T = 0.000 | T = 0.000 | | | |
| 674319 | 80949 | 71501699 | C/T | C = 0.962 | C = 0.969 | 0.618 | 1.24 | T |
| | | | | T = 0.038 | T = 0.031 | | | |
| 675185 | 81130 | 71501880 | T/G | T = 0.237 | T = 0.143 | 0.00179 | 0.54 | T |
| | | | | G = 0.763 | G = 0.857 | | | |
| 5792574 | 81452 | 71502202 | —/C | — = 0.093 | — = 0.044 | 0.0467 | 0.45 | — |
| | | | | C = 0.907 | C = 0.956 | | | |
| 12275272 | 82595 | 71503345 | C/T | C = 0.999 | C = 1 | 0.956 | 1.60 | T |
| | | | | T = 0.001 | T = 0.000 | | | |
| 10400327 | 83633 | 71504383 | C/T | C = 0.001 | C = 0.001 | 1 | 1.01 | |
| | | | | T = 0.999 | T = 0.999 | | | |
| 612255 | 84222 | 71504972 | C/T | C = 0.977 | C = 0.987 | 0.376 | 1.74 | T |
| | | | | T = 0.023 | T = 0.013 | | | |
| 7101643 | 84378 | 71505128 | G/A | G = 1 | G = 0.999 | 0.887 | 0.00 | G |
| | | | | A = 0 | A = 0.001 | | | |
| 575871 | 84380 | 71505130 | A/G | A = 0.078 | A = 0.069 | 0.619 | 0.88 | A |
| | | | | G = 0.922 | G = 0.931 | | | |
| 547208 | 85226 | 71505976 | C/T | C = 0.952 | C = 0.965 | 0.479 | 1.38 | T |
| | | | | T = 0.048 | T = 0.035 | | | |
| 2511075 | 85815 | 71506565 | T/C | T = 0.112 | T = 0.055 | 0.0283 | 0.46 | T |
| | | | | C = 0.888 | C = 0.945 | | | |
| 642573 | 86412 | 71507162 | C/G | C = 0.95 | C = 0.976 | 0.0589 | 2.14 | G |
| | | | | G = 0.050 | G = 0.024 | | | |
| 482197 | 86805 | 71507555 | G/A | G = 0.982 | G = 0.991 | 0.403 | 1.98 | A |
| | | | | A = 0.018 | A = 0.009 | | | |
| 656640 | 87268 | 71508018 | T/C | T = 0.391 | T = 0.348 | 0.194 | 0.83 | T |
| | | | | C = 0.609 | C = 0.652 | | | |
| 671681 | 88370 | 71509120 | C/T | C = 0.929 | C = 0.958 | 0.0747 | 1.75 | T |
| | | | | T = 0.071 | T = 0.042 | | | |
| 541022 | 88614 | 71509364 | A/G | A = 0.929 | A = 0.964 | 0.0379 | 1.99 | G |
| | | | | G = 0.071 | G = 0.036 | | | |
| 951586 | 89223 | 71509973 | A/G | A = 0.003 | A = 0.002 | 0.86 | 0.46 | A |
| | | | | G = 0.997 | G = 0.998 | | | |
| 2511076 | 89385 | 71510135 | G/A | G = 0.778 | G = 0.742 | 0.263 | 0.82 | G |
| | | | | A = 0.222 | A = 0.258 | | | |
| 11235432 | 89500 | 71510250 | G/C | G = 0.997 | G = 0.997 | 0.981 | 0.94 | G |
| | | | | C = 0.003 | C = 0.003 | | | |
| 3018308 | 89502 | 71510252 | T/C | T = 0.629 | T = 0.633 | 0.898 | 1.02 | C |
| | | | | C = 0.371 | C = 0.367 | | | |
| 11606798 | 89929 | 71510679 | G/C | G = 0.997 | G = 0.994 | 0.768 | 0.58 | G |
| | | | | C = 0.003 | C = 0.006 | | | |
| 1791544 | 90515 | 71511265 | C/T | C = 0.102 | C = 0.051 | 0.011 | 0.48 | C |
| | | | | T = 0.898 | T = 0.949 | | | |
| 3018304 | 90659 | 71511409 | C/A | C = 0.996 | C = 0.994 | 0.838 | 0.70 | C |
| | | | | A = 0.004 | A = 0.006 | | | |
| 10751193 | 90914 | 71511664 | A/C | A = 0.738 | A = 0.708 | 0.381 | 0.86 | A |
| | | | | C = 0.262 | C = 0.292 | | | |
| 4945470 | 91594 | 71512344 | C/T | C = 0.999 | C = 0.999 | 0.947 | 0.68 | C |
| | | | | T = 0.001 | T = 0.001 | | | |
| 3018291 | 91603 | 71512353 | A/C | A = 0.994 | A = 0.999 | 0.683 | 3.72 | C |
| | | | | C = 0.006 | C = 0.001 | | | |
| 2511120 | 91769 | 71512519 | A/G | A = 0.989 | A = 0.997 | 0.493 | 3.26 | G |
| | | | | G = 0.011 | G = 0.003 | | | |
| 671132 | 92005 | 71512755 | G/A | G = 0.043 | G = 0.041 | 0.898 | 0.95 | G |
| | | | | A = 0.957 | A = 0.959 | | | |
| 4945475 | 93335 | 71514085 | G/C | G = 0.998 | G = 0.999 | 0.922 | 1.79 | C |
| | | | | C = 0.002 | C = 0.001 | | | |
| 3018292 | 93443 | 71514193 | T/G | T = 1 | T = 1 | 0.993 | 1.38 | G |
| | | | | G = 0.000 | G = 0.000 | | | |
| 642618 | 93751 | 71514501 | C/T | C = 0.1 | C = 0.051 | 0.00594 | 0.48 | C |
| | | | | T = 0.900 | T = 0.949 | | | |
| 552966 | 93775 | 71514525 | A/C | A = 0.159 | A = 0.098 | 0.0096 | 0.57 | A |
| | | | | C = 0.841 | C = 0.902 | | | |
| 6592459 | 94351 | 71515101 | T/G | T = 0.003 | T = 0.004 | 0.903 | 1.63 | G |
| | | | | G = 0.997 | G = 0.996 | | | |
| 607446 | 94810 | 71515560 | C/T | C = 0.142 | C = 0.084 | 0.00611 | 0.55 | C |
| | | | | T = 0.858 | T = 0.916 | | | |
| 607070 | 94856 | 71515606 | A/G | A = 0 | A = 0.001 | 0.972 | | G |
| | | | | G = 1 | G = 0.999 | | | |
| 10713307 | 95893 | 71516643 | C/— | C = 0.662 | C = | | | |
| | | | | — = 0.338 | — = | | | |
| 3018302 | 96500 | 71517250 | T/G | T = 0.233 | T = 0.174 | 0.0339 | 0.69 | T |
| | | | | G = 0.767 | G = 0.826 | | | |
| 3750909 | 97587 | 71518337 | G/C | G = 0.998 | G = 0.998 | 0.986 | 0.93 | G |
| | | | | C = 0.002 | C = 0.002 | | | |
| 3018301 | 97629 | 71518379 | A/G | A = 0.268 | A = 0.167 | 0.000211 | 0.55 | A |
| | | | | G = 0.732 | G = 0.833 | | | |
| 2511114 | 97705 | 71518455 | C/T | C = 0.921 | C = 0.959 | 0.0268 | 1.99 | T |
| | | | | T = 0.079 | T = 0.041 | | | |
| 12270166 | 97967 | 71518717 | A/G | A = | A = 0.193 | | | |
| | | | | G = | G = 0.807 | | | |
| 12270241 | 98126 | 71518876 | G/A | G = 0.999 | G = 0.999 | 0.948 | 0.73 | G |
| | | | | A = 0.001 | A = 0.001 | | | |
| 11606587 | 98422 | 71519172 | A/G | A = 0.999 | A = 0.999 | 0.989 | 1.06 | G |
| | | | | G = 0.001 | G = 0.001 | | | |
| 686340 | 98770 | 71519520 | G/C | G = 0.972 | G = 0.994 | 0.0558 | 4.34 | C |
| | | | | C = 0.028 | C = 0.006 | | | |
| 548961 | 99445 | 71520195 | G/A | G = 0.153 | G = 0.093 | 0.0091 | 0.57 | G |
| | | | | A = 0.847 | A = 0.907 | | | |
| 549032 | 99467 | 71520217 | C/A | C = 0.002 | C = 0.001 | 0.943 | 0.56 | C |
| | | | | A = 0.998 | A = 0.999 | | | |
| 575831 | 100104 | 71520854 | A/G | A = 0.056 | A = 0.043 | 0.393 | 0.75 | A |
| | | | | G = 0.944 | G = 0.957 | | | |
| 575878 | 100121 | 71520871 | A/T | A = 0.001 | A = 0 | 0.948 | 0.00 | A |
| | | | | T = 0.999 | T = 1 | | | |
| 577435 | 100239 | 71520989 | T/C | T = 0.981 | T = 0.985 | 0.717 | 1.26 | C |
| | | | | C = 0.019 | C = 0.015 | | | |
| 579320 | 100512 | 71521262 | G/T | G = 0.092 | G = 0.048 | 0.0151 | 0.50 | G |
| | | | | T = 0.908 | T = 0.952 | | | |
| 495567 | 101046 | 71521796 | C/T | C = 0.11 | C = 0.052 | 0.0205 | 0.44 | C |
| | | | | T = 0.890 | T = 0.948 | | | |
| 636946 | 101267 | 71522017 | C/T | C = 0.999 | C = 0.998 | 0.897 | 0.61 | C |
| | | | | T = 0.001 | T = 0.002 | | | |
| 493065 | 102487 | 71523237 | A/G | A = 0.177 | A = 0.096 | 0.000749 | 0.49 | A |
| | | | | G = 0.823 | G = 0.904 | | | |
| 597513 | 102980 | 71523730 | A/T | A = 0.094 | A = 0.0650 | 0.257 | 0.67 | A |
| | | | | T = 0.906 | T = 0.935 | | | |
| 598835 | 103285 | 71524035 | T/C | T = 0.927 | T = 0.957 | 0.0701 | 1.74 | C |
| | | | | C = 0.073 | C = 0.043 | | | |
| 10683614 | 103359 | 71524109 | —/TAGT | — = 0.027 | — = 0.005 | 0.154 | 0.16 | — |
| | | | | TAGT = 0.973 | TAGT = 0.995 | | | |
| 610004 | 103497 | 71524247 | T/C | T = 0.912 | T = 0.955 | 0.0119 | 2.03 | C |
| | | | | C = 0.088 | C = 0.045 | | | |
| 610041 | 103526 | 71524276 | A/G | A = 0.127 | A = 0.07 | 0.00619 | 0.52 | A |
| | | | | G = 0.873 | G = 0.930 | | | |
| 673478 | 104662 | 71525412 | T/C | T = 0.842 | T = 0.896 | 0.0173 | 1.61 | C |
| | | | | C = 0.158 | C = 0.104 | | | |
| 670802 | 105226 | 71525976 | T/G | T = 0.128 | T = 0.082 | 0.0238 | 0.61 | T |
| | | | | G = 0.872 | G = 0.918 | | | |
| 505041 | 105950 | 71526700 | T/A | T = 0.006 | T = 0.003 | 0.766 | 0.53 | T |
| | | | | A = 0.994 | A = 0.997 | | | |
| 2511116 | 107718 | 71528468 | C/T | C = 0.107 | C = 0.042 | 0.0154 | 0.37 | C |
| | | | | T = 0.893 | T = 0.958 | | | |
| 628025 | 107909 | 71528659 | A/C | A = 0.104 | A = 0.078 | 0.3 | 0.73 | A |
| | | | | C = 0.896 | C = 0.922 | | | |
| 517837 | 107917 | 71528667 | C/T | C = 0.902 | C = 0.935 | 0.075 | 1.56 | T |
| | | | | T = 0.098 | T = 0.065 | | | |
| 615000 | 108510 | 71529260 | T/G | T = 0.19 | T = 0.086 | 0.0000392 | 0.40 | T |
| | | | | G = 0.810 | G = 0.914 | | | |
| 482013 | 109520 | 71530270 | C/T | C = 0.122 | C = 0.077 | 0.0251 | 0.60 | C |
| | | | | T = 0.878 | T = 0.923 | | | |
| 693391 | 109676 | 71530426 | T/G | T = 1 | T = 1 | 0.98 | 1.70 | G |
| | | | | G = 0.000 | G = 0.000 | | | |
| 2511079 | 109712 | 71530462 | T/C | T = 0.934 | T = 0.966 | 0.0397 | 1.99 | C |
| | | | | C = 0.066 | C = 0.034 | | | |
| 2250866 | 110071 | 71530821 | T/C | T = 0.15 | T = 0.085 | 0.00294 | 0.52 | T |
| | | | | C = 0.850 | C = 0.915 | | | |
| 2508860 | 110610 | 71531360 | A/G | A = 0.094 | A = 0.072 | 0.29 | 0.75 | A |
| | | | | G = 0.906 | G = 0.928 | | | |
| 7483267 | 110757 | 71531507 | A/G | A = 0.116 | A = 0.12 | 0.838 | 1.05 | G |
| | | | | G = 0.884 | G = 0.880 | | | |
| 2511078 | 110758 | 71531508 | G/A | G = 0.757 | G = 0.75 | 0.793 | 0.96 | G |
| | | | | A = 0.243 | A = 0.250 | | | |
| 2508859 | 110923 | 71531673 | T/G | T = 1 | T = 1 | 0.987 | 170.03 | G |
| | | | | G = 0.000 | G = 0.000 | | | |
| 2508858 | 110963 | 71531713 | C/G | C = 0.101 | C = 0.057 | 0.0243 | 0.54 | C |
| | | | | G = 0.899 | G = 0.943 | | | |
| 11235435 | 111245 | 71531995 | T/G | T = 0 | T = 0.001 | 0.954 | | G |
| | | | | G = 1 | G = 0.999 | | | |
| 11235436 | 111437 | 71532187 | A/G | A = 0 | A = 0.002 | 0.89 | | G |
| | | | | G = 1 | G = 0.998 | | | |
| 639435 | 112174 | 71532924 | A/C | A = 1 | A = 1 | 0.959 | 0.15 | A |
| | | | | C = 0.000 | C = 0.000 | | | |
| 12285624 | 112646 | 71533396 | A/G | A = 0.505 | A = 0.51 | 0.857 | 1.02 | G |
| | | | | G = 0.495 | G = 0.490 | | | |
| NUMA1-AD | 112784 | 71533534 | G/T | G = 0.005 | G = 0.004 | 0.908 | 0.79 | G |
| | | | | T = 0.995 | T = 0.996 | | | |
| 624363 | 113275 | 71534025 | T/C | T = 0.002 | T = 0.002 | 0.995 | 1.06 | C |
| | | | | C = 0.998 | C = 0.998 | | | |
| NUMA1-AE | 113614 | 71534364 | T/C | T = 0.574 | T = 0.583 | 0.779 | 1.04 | C |
| | | | | C = 0.426 | C = 0.417 | | | |
| 1053573 | 113965 | 71534715 | G/T | G = 0.001 | G = 0 | 0.934 | 0.00 | G |
| | | | | T = 0.999 | T = 1 | | | |
| 1053511 | 114095 | 71534845 | C/T | C = 1 | C = 1 | 1 | 1.00 | |
| | | | | T = 0.000 | T = 0.000 | | | |
| 1063863 | 114125 | 71534875 | G/A | G = 1 | G = 1 | 0.972 | 0.61 | G |
| | | | | A = 0.000 | A = 0.000 | | | |
| 12137 | 114213 | 71534963 | C/T | C = 0.995 | C = 0.99 | 0.624 | 0.55 | C |
| | | | | T = 0.005 | T = 0.010 | | | |
| 4365081 | 114278 | 71535028 | A/G | A = 0.002 | A = 0.002 | 0.983 | 1.16 | G |
| | | | | G = 0.998 | G = 0.998 | | | |
| 4466868 | 114321 | 71535071 | G/C | G = 0.999 | G = 1 | 0.898 | | C |
| | | | | C = 0.001 | C = 0 | | | |
| 3750911 | 114465 | 71535215 | A/G | A = 0.004 | A = 0.004 | 0.985 | 1.06 | G |
| | | | | G = 0.996 | G = 0.996 | | | |
| 510925 | 115058 | 71535808 | T/C | T = 0.099 | T = 0.099 | 0.996 | 1.00 | T |
| | | | | C = 0.901 | C = 0.901 | | | |
| 595062 | 115193 | 71535943 | A/G | A = 0.077 | A = 0.059 | 0.302 | 0.75 | A |
| | | | | G = 0.923 | G = 0.941 | | | |
| 1053443 | 115433 | 71536183 | C/T | C = 0.997 | C = 1 | 0.757 | 4.73 | T |
| | | | | T = 0.003 | T = 0.000 | | | |
| 542752 | 116202 | 71536952 | A/T | A = 0.118 | A = 0.087 | 0.298 | 0.71 | A |
| | | | | T = 0.882 | T = 0.913 | | | |
| 3897579 | 116328 | 71537078 | T/C | T = 0.001 | T = 0.001 | 0.985 | 1.62 | C |
| | | | | C = 0.999 | C = 0.999 | | | |
| 11235437 | 116812 | 71537562 | T/C | T = 0.001 | T = 0.001 | 0.949 | 0.41 | T |
| | | | | C = 0.999 | C = 0.999 | | | |
| 2508856 | 117201 | 71537951 | C/T | C = 0.926 | C = 0.939 | 0.448 | 1.23 | T |
| | | | | T = 0.074 | T = 0.061 | | | |
| 5792575 | 117565 | 71538315 | A/— | A = | A = 0.996 | | | |
| | | | | — = | — = 0.004 | | | |
| 659513 | 117969 | 71538719 | G/A | G = 0.949 | G = 0.976 | 0.0509 | 2.20 | A |
| | | | | A = 0.051 | A = 0.024 | | | |
| 10898820 | 118150 | 71538900 | A/G | A = 0.685 | A = 0.668 | 0.583 | 0.93 | A |
| | | | | G = 0.315 | G = 0.332 | | | |
| 2276397 | 127729 | 71548479 | C/T | C = 0.014 | C = 0.008 | 0.55 | 0.57 | C |
| | | | | T = 0.986 | T = 0.992 | | | |
| 3750908 | 127959 | 71548709 | C/T | C = 0.089 | C = 0.058 | 0.0865 | 0.63 | C |
| | | | | T = 0.911 | T = 0.942 | | | |
| 3793938 | 128691 | 71549441 | C/T | C = 0.915 | C = 0.948 | 0.0698 | 1.69 | T |
| | | | | T = 0.085 | T = 0.052 | | | |
| 602285 | 129086 | 71549836 | C/G | C = 1 | C = 1 | 0.999 | 1.01 | G |
| | | | | G = 0.000 | G = 0.000 | | | |
| 2276396 | 129463 | 71550213 | G/C | G = 0.108 | G = 0.0640 | 0.0188 | 0.56 | G |
| | | | | C = 0.892 | C = 0.936 | | | |
| 2276395 | 129900 | 71550650 | T/C | T = 0.001 | T = 0.002 | 0.902 | 3.67 | C |
| | | | | C = 0.999 | C = 0.998 | | | |
| 1806778 | 136610 | 71557360 | T/C | T = 0.955 | T = 0.969 | 0.341 | 1.44 | C |
| | | | | C = 0.045 | C = 0.031 | | | |
| 4073394 | 137797 | 71558547 | A/G | A = 0.46 | A = 0.422 | 0.23 | 0.86 | A |
| | | | | G = 0.540 | G = 0.578 | | | |
| 471547 | 151737 | 71572487 | G/T | G = 0.488 | G = 0.48 | 0.842 | 0.97 | G |
| | | | | T = 0.512 | T = 0.520 | | | |
| 606136 | 152130 | 71572880 | A/G | A = 0.559 | A = 0.535 | 0.447 | 0.91 | A |
| | | | | G = 0.441 | G = 0.465 | | | |
| 605241 | 152332 | 71573082 | T/C | T = 0.005 | T = 0.002 | 0.712 | 0.35 | T |
| | | | | C = 0.995 | C = 0.998 | | | |
| 686063 | 153789 | 71574539 | G/A | G = 0.003 | G = 0.006 | 0.742 | 2.10 | A |
| | | | | A = 0.997 | A = 0.994 | | | |
| 685749 | 153813 | 71574563 | T/C | T = 0.033 | T = 0.038 | 0.837 | 1.15 | C |
| | | | | C = 0.967 | C = 0.962 | | | |
| 533207 | 153844 | 71574594 | A/C | A = 0.923 | A = 0.919 | 0.811 | 0.94 | A |
| | | | | C = 0.077 | C = 0.081 | | | |
| 476753 | 188612 | 71609362 | A/G | A = 0.077 | A = 0.07 | 0.71 | 0.90 | A |
| | | | | G = 0.923 | G = 0.930 | | | |
| NUMA1-AF | 191149 | 71611899 | G/T | G = 0.963 | G = 0.974 | 0.398 | 1.46 | T |
| | | | | T = 0.037 | T = 0.026 | | | |
| 11278712 | 34468-34472 | 71455218\hat{ }71455222 | —/ | — = 0.901 | — = 0.954 | 0.00321 | 2.24 | GTCAAC |
| | | | GTCA | GTCAAC = 0.099 | GTCAAC = 0.046 | | | |
| | | | AC | | | | | |
| 3831387 | 38627-38628 | 71459377\hat{ }71459378 | CA/— | CA = 0.04 | CA = 0.013 | 0.067 | 0.32 | CA |
| | | | | — = 0.960 | — = 0.987 | | | |

FIG. 1D shows proximal SNPs in and around the NUMA1 region for females. The position of each SNP on the chromosome is presented on the x-axis. They-axis gives the negative logarithm (base 10) of the p-value comparing the estimated allele in the case group to that of the control group. The minor allele frequency of the control group for each SNP designated by an X or other symbol on the graphs in FIG. 1D can be determined by consulting Table 30. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Wed at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

Additional Genotyping

Three additional NUMA1 region SNPs were genotyped in the discovery cohort. The discovery cohort is described in Example 1. The three SNPs (rs1894003, rs675185, and rs615000) map to the following respective locations within the NUMA1 region: 3′UTR of LOC220074, intron of NUMA1, and intron of LOC220074. All three of the SNPs (rs1894003, rs675185, and rs615000) were found to be significantly associated with breast cancer with a p-value of 0.00833, 0.0465 and 0.00648, respectively. See Table 33.

The methods used to verify and genotype the three proximal SNPs of Table 26 are the same methods described in Examples 1 and 2 herein. The PCR primers and extend primers used in these assays are provided in Table 31 (SEQ ID NOS 2194-2199, respectively, in order of appearance) and Table 32 (SEQ ID NOS 2200-2202, respectively, in order of appearance), respectively.

TABLE 31

| dbSNP rs# | Forward PCR primer | Reverse PCR primer |
|---|---|---|
| 1894003 | ACGTTGGATGGCCTAGCCA | ACGTTGGATGTTGACCCTCT |
| | TGATTTCCTAC | GGTCCTGC |
| 675185 | ACGTTGGATGCCCGTCTCT | ACGTTGGATGCCCAGCCTAT |
| | GGGGAAAAAAA | ATCCTGCTTT |
| 615000 | ACGTTGGATGATTTTGGCA | ACGTTGGATGGCCTCCATTC |
| | ACTTAGAAGTC | ACTCTTGAAC |

TABLE 32

| dbSNP rs# | Extend Primer | Term Mix |
|---|---|---|
| 1894003 | TGTAAACCACCTGCTCAGAC | ACT |
| 675185 | ATGGGGAAATACTCAAGAAA | CT |
| 615000 | CCTGGAGGCAGAAGCCAGAT | ACT |

Table 26, below, shows the case and control allele frequencies along with the p-values for additional SNPs genotyped. The disease associated allele of column 4 is in bold. The chromosome positions provided correspond to NCBI's Build 34.

TABLE 33
Genotyping Results

| | Position in | | | Amino | | | | | |
|---|---|---|---|---|---|---|---|---|---|
| | SEQ ID | Chromosome | Alleles | Acid | | AF F | | Odds | Breast Cancer |
| dbSNP rs# | NO: 4 | Position | (A1/A2) | Change | AF F case | control | p-value | Ratio | Assoc. Allele |
| 1894003 | 55834 | 71476584 | T/C | | T = 0.086 | T = 0.045 | 0.00833 | 0.504 | T |
| | | | | | C = 0.914 | C = 0.955 | | | |
| 675185 | 81130 | 71501880 | T/G | | T = 0.076 | T = 0.046 | 0.0465 | 0.587 | T |
| | | | | | G = 0.924 | G = 0.954 | | | |
| 615000 | 108510 | 71529260 | T/G | | T = 0.085 | T = 0.043 | 0.00648 | 0.486 | T |
| | | | | | G = 0.915 | G = 0.957 | | | |

### Example 8

### Meta Analysis of Incident SNPs

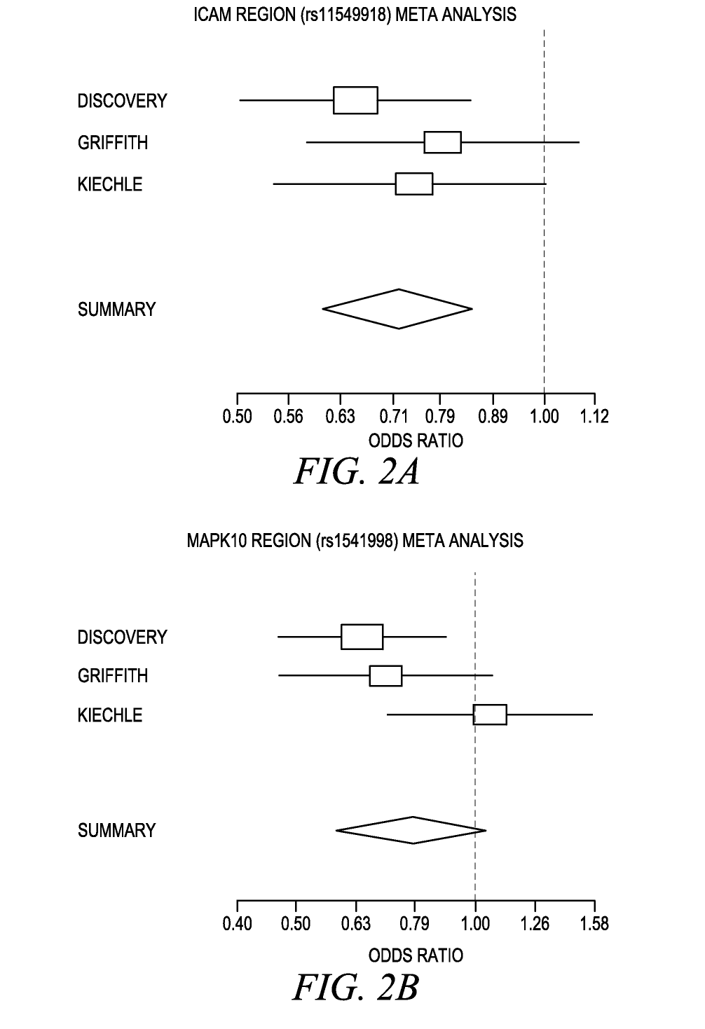

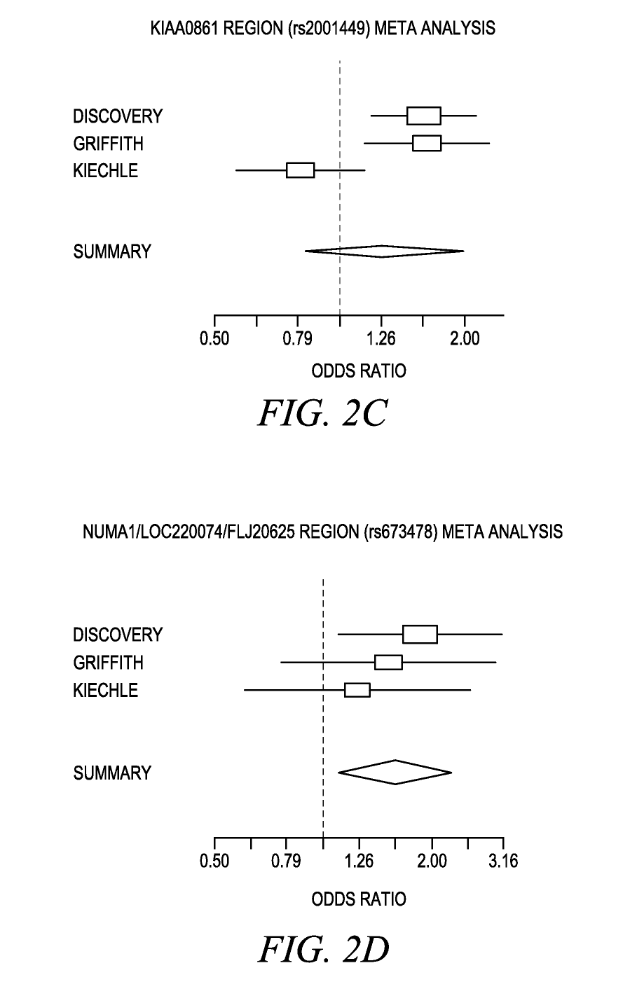

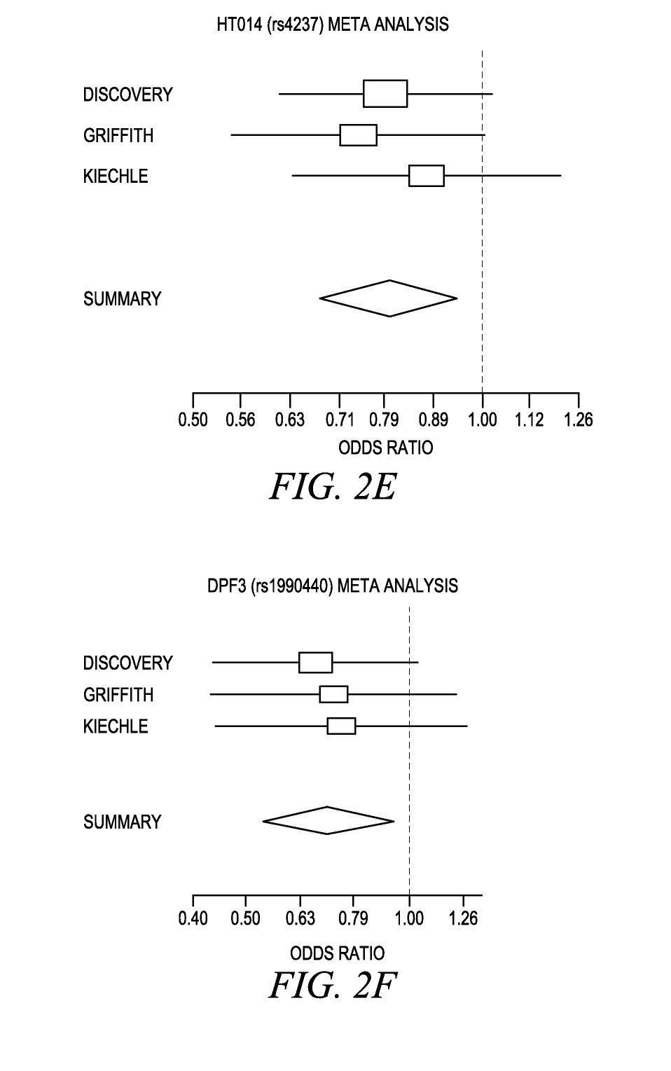

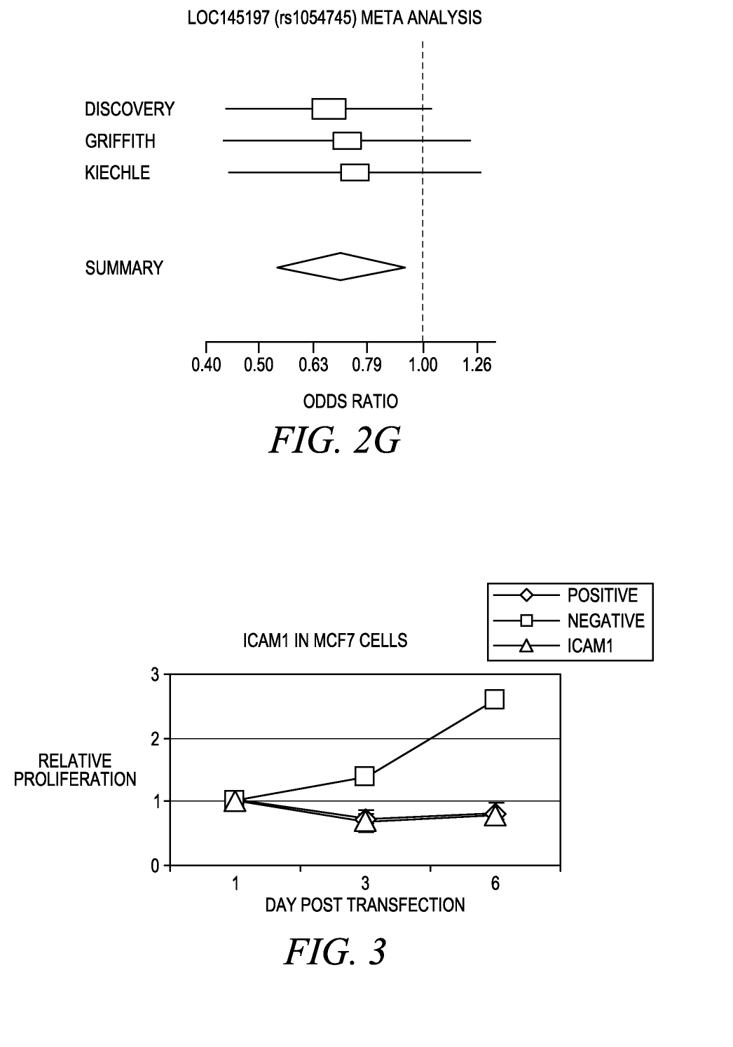

Meta-analysis was performed of five of the incident SNPs disclosed in Table 3 (ICAM region (ICAM_SNP), MAPK10 (rs1541998), KIAA0861 (rs2001449), NUMA1 region (rs673478) and GALE region (rs4237)) based on genotype results provided in Table 6B. FIGS. 2A-2D depict odds ratios for the discovery samples and replication samples (see Example 3) individually, and the combined meta analysis odds ratio for the named SNP. The boxes are centered over the odds ratio for each sample, with the size of the box correlated to the contribution of each sample to the combined meta analysis odds ratio. The lines extending from each box are the 95% confidence interval values. The diamond is centered over the combined meta analysis odds ratio with the ends of the diamond depicting the 95% confidence interval values. The meta-analysis further illustrates the strong association each of the incident SNPs has with breast cancer across multiple case and control samples.

The subjects available for discovery from Germany included 272 cases and 276 controls. The subjects available for replication from Australia included 190 breast cancer cases and 190 controls. Meta analyses, combining the results of the German discovery sample and the Australian replication sample, were carried out using a random effects (DerSimonian-Laird) procedure.

### Example 9

### Description of Development of Predictive Breast Cancer Models

The five SNPs reported in Example 3 were identified as being significantly associated with breast cancer according to the replication analysis discussed therein. These five SNPs are a subset of the panel of SNPs associated with breast cancer in the German chort referenced in Example 1 and reported in provisional patent application No. 60/429,136 filed Nov. 25, 2002 and provisional patent application No. 60/490,234 filed Jul. 24, 2003, having attorney docket number 524593004100 and 524593004101, respectively.

The clinical importance of these SNPs was estimated by combining them into a single logistic regression model. The coefficients of the model were used to estimate penetrance, relative risk and odds ratio values for estimating a subject's risk of having or developing breast cancer according to the subject's genotype. Penetrance is a probability that an individual has or will have breast cancer given their genotype (e.g., a value of 0.01 in the tables is equal to a 1% chance of having or developing breast cancer). The relative risk of breast cancer is based upon penetrance values, and is expressed in two forms. One form, noted as RR in the tables below, is expressed as a risk with respect to the lowest risk group (e.g., the most protected group being the 00000 genotype listed in Table 33). The other form is expressed as a risk with respect to a population average risk of breast cancer, which is noted as RR(Pop) in Table 35 below. Both of these expressions of relative risk are useful to a clinician for assessing risk of breast cancer in an individual and targeting appropriate detection, prevention and/or treatment regimens to the subject. Both expressions of relative risk also are useful to an insurance company to assess population risks of breast cancer (e.g., for developing actuarial tables), where individual genotypes often are provided to the company on an anonymous basis. Odds ratios are the odds one group has or will develop breast cancer with respect to another group, the other group often being the most protective group or the group having a population average risk of breast cancer. Relative risk often is a more reliable assessment of risk in comparison to an odds ratio when the disease or condition at issue is more prevalent.

To fit the single logistic model, all cases and controls from the German and Australian samples were used (see Examples 1 and 3, respectively). Controls were coded as 0 and cases were coded as 1. Based on the genotype penetrance estimates of each SNP (Table 31), GP01.025495354 (rs4237), GP03.197942797 (rs2001449), GP11.079035103 (rs673478) were modeled as additive by coding the genotypes 0, 1, or 2 for the low risk homozygote, the heterozygote, or high risk homozygote, respectively. The SNP FCH.0994 (ICAM_SNP1) was modeled as recessive coding the genotypes 0, 0, or 2 for the low risk homozygote, heterozygote, or high risk homozygote, respectively. The SNP GP04.091348915 (rs 1541998) was modeled as dominant coding the genotypes 0, 2, or 2 for the low risk homozygote, the heterozygote, or high risk homozygote, respectively. Table 31 summarizes this analysis.

TABLE 31

| SNP: | | | Case | | | |
|---|---|---|---|---|---|---|
| Genotype | | N | (N = 254) | Control (N = 268) | P(D\|G) (%) | P-value |
| ICAM_SNP1: | CC | 497 | 45% (103) | 32% (85) | 4.140 | 0.006210 |
| | CT | | 42% (98) | 47% (126) | 2.700 | |
| | TT | | 13% (30) | 21% (13) | 1.910 | |
| rs4237: | AA | 494 | 34% (79) | 29% (75) | 3.550 | 0.186000 |
| | AG | | 49% (113) | 48% (126) | 3.040 | |
| | GG | | 17% (40) | 23% (61) | 2.240 | |
| rs2001449: | GG | 508 | 46% (112) | 60% (158) | 2.280 | 0.002930 |
| | GC | | 48% (117) | 36% (94) | 3.940 | |
| | CC | | 7% (17) | 4% (10) | 5.300 | |
| rs673478: | TT | 509 | 84% (206) | 91% (240) | 2.800 | 0.040700 |
| | TC | | 14% (35) | 9% (25) | 4.490 | |
| | CC | | 1% (3) | 0% (0) | 100.00 | |
| rs1541998: | CC | 493 | 5% (12) | 4% (10) | 3.710 | 0.012100 |
| | CT | | 36% (87) | 24% (61) | 4.370 | |
| | TT | | 59% (143) | 72% (180) | 2.490 | |

Based on this coding, there are a total of 108 unique genotype codes from the 243 unique five SNP genotypes. The relationship between the five SNP genotypes and the case-control status was fit using logistic regression. Many models were fit and compared including the five SNPs and all possible interaction among SNPs and study center. Only statistically significant terms from this complete model were included in the final model, shown in Table 32.

TABLE 32

| | Estimate | Std. Error | z value | Pr(>\|z\|) |
|---|---|---|---|---|
| (Intercept) | −1.34446 | 0.25972 | −5.177 | 2.26e−07 |
| FCH.0994 | 0.77607 | 0.19835 | 3.913 | 9.13e−05 |
| 4237 | 0.54525 | 0.17666 | 3.086 | 0.002025 |
| 2001449 | 0.60383 | 0.28487 | 2.120 | 0.034033 |
| 1541998 | 0.22051 | 0.07849 | 2.809 | 0.004963 |
| 673478 | 0.59961 | 0.21737 | 2.758 | 0.005807 |
| FCH.0994c: 4237 | −0.52636 | 0.14516 | −3.626 | 0.000288 |
| FCH.0994c: 2001449 | −0.35613 | 0.24503 | −1.453 | 0.146113 |
| 4237c: 2001449 | −0.15685 | 0.20191 | −0.777 | 0.437257 |
| FCH.0994c: 4237c2001449 | 0.41305 | 0.18391 | 2.246 | 0.024705 |

Null deviance: 1136.7 on 820 degrees of freedom
Residual deviance: 1069.6 on 811 degrees of freedom
AIC: 1089.6

The penetrance was calculated for each of the 108 unique genotype codes using this model and an assumed disease prevalence of 0.03 (prev), the cumulative incidence for the age range of the sample in question. This was calculated from the logistic model as follows:

penetrance=exp(ŷ+adj)/(1+exp(ŷ+adj))

where

ŷ=1/(1+exp(−1.344+0.776*A+0.545*B+0.604*C+0.221*D+0.600*E−0.526*A*B−0.356*A*C−0.157*B*C+0.413*A*B*C))

and

adj=ln(prev/(1−prev)*freq(case)/(1−freq(case)).

Here A, B, C, D, and E refer to the genotype codes for the SNPs FCH.0994, 4237, 2001449, 1541998, and 673478, respectively.

Table 33 summarizes statistics of interest for each genotype code. “Geno” shows each genotype code with the five integer codes formatted as an integer string. “N Case” and “N Control” is the number of cases and controls with the specified code, respectively. “Frequency” is the expected percent of individuals in the population having that code calculated as the average of the case and control frequencies weighted by the probability of disease in this sample (0.03). “OR” is the odds ratio comparing the odds of the specified code to the odds of the most protective code (00000) using the parameter estimates from the logistic regression model. “OR (Frq)” is an odds ratio estimated using the frequency of cases and control with the specified genotype code and the most protective code. “RR” is the relative risk comparing the probability of disease of the specified code to the probability of disease of the most protective code. “Penetrance” is the probability of disease given the genotype code, followed by “Lower” and “Upper” which give the 95% confidence interval for the penetrance. As can be seen by the ratios for OR and RR, the 00000 genotype was the most protective against breast cancer occurrence.

TABLE 33

| | | | | | | | | Confidence | |
|---|---|---|---|---|---|---|---|---|---|
| | | | | | | | | Interval | |
| Geno | N Case | N Control | Frequency | OR | OR (Frq) | RR | Penetrance | Lower | Upper |
| 00000 | 6 | 26 | 5.94% | 1.00 | 1.00 | 1.00 | 0.010 | 0.007 | 0.014 |
| 00001 | 0 | 3 | 0.68% | 1.75 | 0.00 | 1.74 | 0.017 | 0.011 | 0.029 |
| 00002 | 0 | 0 | 0.00% | 3.08 | | 3.01 | 0.030 | 0.013 | 0.069 |
| 00020 | 3 | 9 | 2.06% | 1.61 | 1.44 | 1.60 | 0.016 | 0.011 | 0.023 |
| 00021 | 0 | 3 | 0.68% | 2.83 | 0.00 | 2.78 | 0.028 | 0.017 | 0.047 |
| 00022 | 0 | 0 | 0.00% | 4.97 | | 4.78 | 0.048 | 0.021 | 0.108 |
| 00100 | 9 | 20 | 4.60% | 1.67 | 1.95 | 1.66 | 0.017 | 0.012 | 0.023 |
| 00101 | 2 | 1 | 0.24% | 2.93 | 8.67 | 2.87 | 0.029 | 0.018 | 0.047 |
| 00102 | 0 | 0 | 0.00% | 5.13 | | 4.93 | 0.050 | 0.022 | 0.110 |
| 00120 | 7 | 6 | 1.41% | 2.69 | 5.06 | 2.65 | 0.027 | 0.018 | 0.038 |
| 00121 | 0 | 0 | 0.00% | 4.73 | | 4.56 | 0.046 | 0.028 | 0.075 |
| 00122 | 0 | 0 | 0.00% | 8.29 | | 7.72 | 0.078 | 0.034 | 0.168 |
| 00200 | 1 | 4 | 0.91% | 2.78 | 1.08 | 2.74 | 0.027 | 0.018 | 0.042 |
| 00201 | 0 | 0 | 0.00% | 4.88 | | 4.70 | 0.047 | 0.027 | 0.082 |
| 00202 | 0 | 0 | 0.00% | 8.57 | | 7.96 | 0.080 | 0.034 | 0.178 |
| 00220 | 1 | 1 | 0.23% | 4.50 | 4.33 | 4.34 | 0.044 | 0.027 | 0.070 |
| 00221 | 1 | 0 | 0.01% | 7.89 | | 7.38 | 0.074 | 0.041 | 0.129 |
| 00222 | 0 | 0 | 0.00% | 13.83 | | 12.25 | 0.123 | 0.052 | 0.263 |
| 01000 | 24 | 47 | 10.84% | 1.26 | 2.21 | 1.26 | 0.013 | 0.010 | 0.016 |
| 01001 | 3 | 1 | 0.25% | 2.21 | 13.00 | 2.18 | 0.022 | 0.014 | 0.034 |
| 01002 | 0 | 0 | 0.00% | 3.87 | | 3.77 | 0.038 | 0.017 | 0.083 |
| 01020 | 18 | 22 | 5.12% | 2.03 | 3.55 | 2.01 | 0.020 | 0.015 | 0.027 |
| 01021 | 4 | 4 | 0.94% | 3.57 | 4.33 | 3.48 | 0.035 | 0.022 | 0.055 |
| 01022 | 0 | 0 | 0.00% | 6.26 | | 5.94 | 0.060 | 0.027 | 0.129 |
| 01100 | 21 | 33 | 7.64% | 2.10 | 2.76 | 2.08 | 0.021 | 0.017 | 0.026 |
| 01101 | 2 | 4 | 0.92% | 3.69 | 2.17 | 3.59 | 0.036 | 0.024 | 0.055 |
| 01102 | 0 | 0 | 0.00% | 6.47 | | 6.13 | 0.062 | 0.028 | 0.130 |
| 01120 | 15 | 6 | 1.47% | 3.39 | 10.83 | 3.31 | 0.033 | 0.025 | 0.045 |
| 01121 | 0 | 0 | 0.00% | 5.95 | | 5.67 | 0.057 | 0.036 | 0.089 |
| 01122 | 0 | 0 | 0.00% | 10.44 | | 9.54 | 0.096 | 0.044 | 0.198 |
| 01200 | 5 | 4 | 0.94% | 3.51 | 5.42 | 3.42 | 0.034 | 0.023 | 0.050 |
| 01201 | 0 | 1 | 0.23% | 6.15 | 0.00 | 5.85 | 0.059 | 0.035 | 0.097 |
| 01202 | 0 | 0 | 0.00% | 10.79 | | 9.82 | 0.099 | 0.044 | 0.209 |
| 01220 | 1 | 0 | 0.01% | 5.66 | | 5.41 | 0.054 | 0.035 | 0.083 |
| 01221 | 0 | 0 | 0.00% | 9.93 | | 9.12 | 0.092 | 0.054 | 0.152 |
| 01222 | 0 | 0 | 0.00% | 17.42 | | 14.95 | 0.150 | 0.067 | 0.304 |
| 02000 | 22 | 39 | 9.01% | 1.59 | 2.44 | 1.58 | 0.016 | 0.012 | 0.021 |
| 02001 | 2 | 1 | 0.24% | 2.78 | 8.67 | 2.73 | 0.027 | 0.017 | 0.043 |
| 02002 | 1 | 0 | 0.01% | 4.88 | | 4.70 | 0.047 | 0.021 | 0.103 |
| 02020 | 16 | 10 | 2.39% | 2.56 | 6.93 | 2.52 | 0.025 | 0.018 | 0.035 |
| 02021 | 2 | 2 | 0.47% | 4.49 | 4.33 | 4.34 | 0.044 | 0.027 | 0.070 |
| 02022 | 2 | 0 | 0.02% | 7.88 | | 7.37 | 0.074 | 0.033 | 0.158 |
| 02100 | 21 | 18 | 4.24% | 2.65 | 5.06 | 2.60 | 0.026 | 0.020 | 0.035 |
| 02101 | 5 | 3 | 0.72% | 4.64 | 7.22 | 4.48 | 0.045 | 0.029 | 0.070 |
| 02102 | 0 | 0 | 0.00% | 8.14 | | 7.60 | 0.076 | 0.035 | 0.160 |
| 02120 | 11 | 8 | 1.90% | 4.28 | 5.96 | 4.14 | 0.042 | 0.030 | 0.058 |
| 02121 | 1 | 0 | 0.01% | 7.50 | | 7.04 | 0.071 | 0.044 | 0.112 |
| 02122 | 0 | 0 | 0.00% | 13.15 | | 11.72 | 0.118 | 0.054 | 0.239 |
| 02200 | 4 | 4 | 0.94% | 4.42 | 4.33 | 4.27 | 0.043 | 0.028 | 0.065 |
| 02201 | 3 | 1 | 0.25% | 7.75 | 13.00 | 7.26 | 0.073 | 0.043 | 0.121 |
| 02202 | 0 | 0 | 0.00% | 13.59 | | 12.06 | 0.121 | 0.053 | 0.252 |
| 02220 | 2 | 1 | 0.24% | 7.13 | 8.67 | 6.72 | 0.068 | 0.043 | 0.106 |
| 02221 | 0 | 0 | 0.00% | 12.51 | | 11.21 | 0.113 | 0.065 | 0.189 |
| 02222 | 0 | 0 | 0.00% | 21.94 | | 18.13 | 0.182 | 0.082 | 0.358 |
| 20000 | 9 | 6 | 1.43% | 1.58 | 6.50 | 1.57 | 0.016 | 0.011 | 0.023 |
| 20001 | 0 | 0 | 0.00% | 2.76 | | 2.72 | 0.027 | 0.016 | 0.045 |
| 20002 | 0 | 0 | 0.00% | 4.85 | | 4.67 | 0.047 | 0.020 | 0.105 |
| 20020 | 8 | 4 | 0.97% | 2.54 | 8.67 | 2.51 | 0.025 | 0.017 | 0.037 |
| 20021 | 0 | 0 | 0.00% | 4.46 | | 4.31 | 0.043 | 0.026 | 0.072 |
| 20022 | 0 | 0 | 0.00% | 7.83 | | 7.33 | 0.074 | 0.032 | 0.161 |
| 20100 | 5 | 6 | 1.40% | 2.63 | 3.61 | 2.59 | 0.026 | 0.018 | 0.037 |
| 20101 | 4 | 1 | 0.26% | 4.61 | 17.33 | 4.45 | 0.045 | 0.027 | 0.072 |
| 20102 | 0 | 0 | 0.00% | 8.09 | | 7.55 | 0.076 | 0.033 | 0.163 |
| 20120 | 4 | 1 | 0.26% | 4.25 | 17.33 | 4.11 | 0.041 | 0.028 | 0.060 |
| 20121 | 1 | 0 | 0.01% | 7.45 | | 6.99 | 0.070 | 0.042 | 0.115 |
| 20122 | 0 | 0 | 0.00% | 13.06 | | 11.65 | 0.117 | 0.052 | 0.242 |
| 20200 | 0 | 1 | 0.23% | 4.39 | 0.00 | 4.24 | 0.043 | 0.027 | 0.066 |
| 20201 | 1 | 0 | 0.01% | 7.70 | | 7.21 | 0.072 | 0.041 | 0.124 |
| 20202 | 0 | 0 | 0.00% | 13.50 | | 11.99 | 0.121 | 0.052 | 0.255 |
| 20220 | 0 | 0 | 0.00% | 7.09 | | 6.68 | 0.067 | 0.041 | 0.108 |
| 20221 | 0 | 0 | 0.00% | 12.43 | | 11.15 | 0.112 | 0.063 | 0.192 |
| 20222 | 0 | 0 | 0.00% | 21.80 | | 18.03 | 0.181 | 0.080 | 0.361 |
| 21000 | 22 | 25 | 5.83% | 1.99 | 3.81 | 1.97 | 0.020 | 0.015 | 0.026 |
| 21001 | 3 | 4 | 0.93% | 3.48 | 3.25 | 3.40 | 0.034 | 0.022 | 0.053 |
| 21002 | 1 | 0 | 0.01% | 6.11 | | 5.81 | 0.058 | 0.026 | 0.125 |
| 21020 | 11 | 14 | 3.26% | 3.21 | 3.40 | 3.14 | 0.032 | 0.023 | 0.043 |
| 21021 | 1 | 2 | 0.46% | 5.62 | 2.17 | 5.37 | 0.054 | 0.034 | 0.085 |
| 21022 | 0 | 0 | 0.00% | 9.86 | | 9.05 | 0.091 | 0.041 | 0.190 |
| 21100 | 26 | 24 | 5.64% | 3.31 | 4.69 | 3.24 | 0.033 | 0.025 | 0.042 |
| 21101 | 1 | 2 | 0.46% | 5.81 | 2.17 | 5.54 | 0.056 | 0.036 | 0.085 |
| 21102 | 1 | 0 | 0.01% | 10.19 | | 9.33 | 0.094 | 0.043 | 0.191 |
| 21120 | 16 | 6 | 1.48% | 5.35 | 11.56 | 5.12 | 0.051 | 0.037 | 0.071 |
| 21121 | 4 | 0 | 0.03% | 9.38 | | 8.65 | 0.087 | 0.055 | 0.135 |
| 21122 | 0 | 0 | 0.00% | 16.45 | | 14.24 | 0.143 | 0.067 | 0.281 |
| 21200 | 3 | 1 | 0.25% | 5.53 | 13.00 | 5.29 | 0.053 | 0.036 | 0.078 |
| 21201 | 3 | 0 | 0.02% | 9.69 | | 8.92 | 0.090 | 0.054 | 0.146 |
| 21202 | 0 | 0 | 0.00% | 17.00 | | 14.65 | 0.147 | 0.067 | 0.295 |
| 21220 | 2 | 2 | 0.47% | 8.93 | 4.33 | 8.27 | 0.083 | 0.053 | 0.127 |
| 21221 | 1 | 0 | 0.01% | 15.65 | | 13.65 | 0.137 | 0.081 | 0.223 |
| 21222 | 0 | 0 | 0.00% | 27.46 | | 21.69 | 0.218 | 0.101 | 0.409 |
| 22000 | 13 | 23 | 5.31% | 2.50 | 2.45 | 2.46 | 0.025 | 0.018 | 0.034 |
| 22001 | 4 | 1 | 0.26% | 4.39 | 17.33 | 4.24 | 0.043 | 0.027 | 0.068 |
| 22002 | 0 | 1 | 0.23% | 7.69 | 0.00 | 7.21 | 0.072 | 0.032 | 0.154 |
| 22020 | 3 | 10 | 2.29% | 4.04 | 1.30 | 3.92 | 0.039 | 0.027 | 0.056 |
| 22021 | 1 | 0 | 0.01% | 7.08 | | 6.67 | 0.067 | 0.041 | 0.107 |
| 22022 | 0 | 0 | 0.00% | 12.42 | | 11.14 | 0.112 | 0.051 | 0.230 |
| 22100 | 15 | 5 | 1.25% | 4.17 | 13.00 | 4.04 | 0.041 | 0.030 | 0.055 |
| 22101 | 1 | 0 | 0.01% | 7.32 | | 6.88 | 0.069 | 0.044 | 0.107 |
| 22102 | 0 | 0 | 0.00% | 12.83 | | 11.47 | 0.115 | 0.053 | 0.232 |
| 22120 | 3 | 5 | 1.16% | 6.74 | 2.60 | 6.37 | 0.064 | 0.045 | 0.091 |
| 22121 | 3 | 1 | 0.25% | 11.82 | 13.00 | 10.66 | 0.107 | 0.066 | 0.168 |
| 22122 | 0 | 0 | 0.00% | 20.72 | | 17.30 | 0.174 | 0.081 | 0.333 |
| 22200 | 4 | 0 | 0.03% | 6.96 | | 6.57 | 0.066 | 0.043 | 0.100 |
| 22201 | 0 | 0 | 0.00% | 12.21 | | 10.97 | 0.110 | 0.065 | 0.181 |
| 22202 | 0 | 0 | 0.00% | 21.42 | | 17.77 | 0.179 | 0.081 | 0.348 |
| 22220 | 4 | 1 | 0.26% | 11.24 | 17.33 | 10.19 | 0.102 | 0.064 | 0.160 |
| 22221 | 0 | 0 | 0.00% | 19.72 | | 16.60 | 0.167 | 0.097 | 0.271 |
| 22222 | 0 | 0 | 0.00% | 34.58 | | 25.86 | 0.260 | 0.122 | 0.470 |

To simplify the interpretation of genotype risk, the 243 unique genotypes were divided into five risk classes on the basis of each estimated penetrance. The levels selected for risk class definitions and the resulting assignment of genotypes into five risk classes is shown in Table 34. The frequency percent of each genotype combination is given in parentheses.

TABLE 34

| Class 1 | Class 2 | Class 3 | Class 4 | Class 5 |
|---|---|---|---|---|
| (0, 0.013] | (0.013, 0.025] | (0.025, 0.042] | (0.042, 0.1] | (0.1, 1) |
| 00000 (5.94) | 00001 (0.68) | 00022 (0.00) | 00102 (0.00) | 00222 (0.00) |
| 00020 (2.06) | 00002 (0.00) | 00121 (0.00) | 00122 (0.00) | 01222 (0.00) |
| 01000 (10.84) | 00021 (0.68) | 00220 (0.23) | 00201 (0.00) | 02022 (0.02) |
| 22000 (5.31) | 00100 (4.60) | 01002 (0.00) | 00202 (0.00) | 02122 (0.00) |
| | 00101 (0.24) | 01021 (0.94) | 00221 (0.01) | 02202 (0.00) |
| | 00120 (1.41) | 01101 (0.92) | 01022 (0.00) | 02221 (0.00) |
| | 00200 (0.91) | 01120 (1.47) | 01102 (0.00) | 02222 (0.00) |
| | 01001 (0.25) | 01200 (0.94) | 01121 (0.00) | 20002 (0.00) |
| | 01020 (5.12) | 02001 (0.24) | 01122 (0.00) | 20022 (0.00) |
| | 01100 (7.64) | 02020 (2.39) | 01201 (0.23) | 20122 (0.00) |
| | 02000 (9.01) | 02100 (4.24) | 01202 (0.00) | 20222 (0.00) |
| | 21000 (5.83) | 02200 (0.94) | 01220 (0.01) | 21102 (0.01) |
| | 22001 (0.26) | 20000 (1.43) | 01221 (0.00) | 21122 (0.00) |
| | 22020 (2.29) | 20100 (1.40) | 02002 (0.01) | 21201 (0.02) |
| | | 20200 (0.23) | 02021 (0.47) | 21202 (0.00) |
| | | 20220 (0.00) | 02101 (0.72) | 21221 (0.01) |
| | | 21001 (0.93) | 02102 (0.00) | 21222 (0.00) |
| | | 21020 (3.26) | 02120 (1.90) | 22102 (0.00) |
| | | 21100 (5.64) | 02121 (0.01) | 22121 (0.25) |
| | | 22002 (0.23) | 02201 (0.25) | 22122 (0.00) |
| | | 22021 (0.01) | 02220 (0.24) | 22200 (0.03) |
| | | 22100 (1.25) | 20001 (0.00) | 22201 (0.00) |
| | | | 20020 (0.97) | 22202 (0.00) |
| | | | 20021 (0.00) | 22220 (0.26) |
| | | | 20101 (0.26) | 22221 (0.00) |
| | | | 20102 (0.00) | 22222 (0.00) |
| | | | 20120 (0.26) | |
| | | | 20121 (0.01) | |
| | | | 20201 (0.01) | |
| | | | 20202 (0.00) | |
| | | | 20221 (0.00) | |
| | | | 21002 (0.01) | |
| | | | 21021 (0.46) | |
| | | | 21022 (0.00) | |
| | | | 21101 (0.46) | |
| | | | 21120 (1.48) | |
| | | | 21121 (0.03) | |
| | | | 21200 (0.25) | |
| | | | 21220 (0.47) | |
| | | | 22022 (0.00) | |
| | | \grave{ } | 22101 (0.01) | |
| | | | 22120 (1.16) | |

With this classification, each genotype was recoded as belonging to their respective class and a logistic regression model was fit with the genotype risk class as a categorical variable. Key summary statistics are summarized in Table 35. Each group is described by the number of cases, number of controls, the estimated risk class population frequency, the odds ratio comparing the odds of the given risk class compared to the odds of the lowest risk class, the penetrance, the relative risk (risk class penetrance divided by most protective risk class penetrance), and the population relative risk (risk class penetrance divided by the disease prevalence: 0.03).

TABLE 35

| | N | N | Frequency | | | | RR |
|---|---|---|---|---|---|---|---|
| Risk Class | Case | Control | (%) | OR | Penetrance | RR | (Pop) |
| G1 | 46 | 105 | 24.2 | 1.0 | 0.012 | 1.0 | 0.41 |
| G2 | 112 | 168 | 38.9 | 1.5 | 0.019 | 1.5 | 0.62 |
| G3 | 140 | 113 | 26.7 | 2.8 | 0.034 | 2.8 | 1.13 |
| G4 | 77 | 40 | 9.7 | 4.4 | 0.052 | 4.2 | 1.73 |
| G5 | 18 | 2 | 0.06 | 20.5 | 0.204 | 16.6 | 6.79 |

### Example 10

### Inhibition of ICAM Gene Expression by Transfection of Specific siRNAs

RNAi-based gene inhibition was selected as a rapid way to inhibit expression of ICAM1 in cultured cells. siRNA reagents were selectively designed to target the ICAM1 gene. Algorithms useful for designing siRNA molecules specific for an ICAM1 gene are disclosed on the World Wide Wed at the URL “dharmacon.com.” siRNA molecules up to 21 nucleotides in length were utilized.

Table 31 summarizes the features of the duplexes that were used in the assays to target ICAM1. A non-homologous siRNA reagent (siGL2 control) was used as a negative control, and a non-homologous siRNA reagent (siRNA_RAD21_{—}1175 control) shown to inhibit the expression of RAD21 and subsequently inhibit cell proliferation was used as a positive control in all of the assays described herein.

TABLE 36

| | siRNA | | SEQ ID |
|---|---|---|---|
| siRNA | Target | Sequence Specificity | NO: |
| ICAM1_293 | ICAM1 | ACAACCGGAAGGUGUAUGA | 4928 |
| ICAM1_335 | ICAM1 | GCCAACCAAUGUGCUAUUC | 4929 |
| ICAM1_604 | ICAM1 | GAUCACCAUGGAGCCAAUU | 4930 |
| ICAM1_1409 | ICAM1 | CUGUCACUCGAGAUCUUGA | 4931 |
| siRNA_RAD21_1175 | RAD21 | GAGUUGGAUAGCAAGACAA | 4932 |
| positive control | | | |
| siGL2 negative | GL2 | CGUACGCGGAAUACUUCGA | 4933 |
| control | | | |

The siRNAs were transfected in cell lines MCF-7 and T-47D using Lipofectamine™ 2000 reagent from Invitrogen, Corp. 2.5 μg or 5.0 μg of siRNA was mixed with 6.25 μl or 12.5 μl lipofectamine, respectively, and the mixture was added to cells grown in 6-well plates. Their inhibitory effects on ICAM1 gene expression were confirmed by precision expression analysis by MassARRAY (quantitativeRT-PCR hME), which was performed on RNA prepared from the transfected cells. See Chunming & Cantor, PNAS 100(6): 3059-3064 (2003). Cell viability was measured at 1, 2, 4 and 6 days post-transfection. Absorbance values were normalized relative to Day 1. RNA was extracted with Trizole reagent as recommended by the manufacturer (Invitrogen, Corp.) followed by cDNA synthesis using SuperScript™ reverse transcriptase.

A cocktail of siRNA molecules described in Table 28 (that target ICAM1) strongly inhibited proliferation of breast cancer cell line (MCF-7), as shown in FIG. 3. These effects are consistent in all six experiments performed. Each data point is an average of 3 wells of a 96-well plate normalized to values obtained from day 1 post transfection. The specificity of the active siRNAs, was confirmed with a negative, non-homologous control siRNA (siGL2), and a positive control, siRNA_RAD21_{—}1175, that targets a known cancer-associated gene, RAD21. Long term inhibition of gene expression is desirable in certain cases. Therefore, included herein are embodiments directed to siRNA duplexes described herein (see Table 36) that are less susceptible to degradation. An example of a modification that decreases susceptibility to degradation is in siSTABLE RNA described on the World Wide Wed at the URL “dharmacon.com.”

### Example 11

### In Vitro Production of Target Polypeptides

cDNA is cloned into a pIVEX 2.3-MCS vector (Roche Biochem) using a directional cloning method. A cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the target polypeptide for the purpose of protein purification. Target polypeptide is purified by contacting the contents of reaction device with resin modified with Ni²⁺ ions. Target polypeptide is eluted from the resin with a solution containing free Ni²⁺ ions.

### Example 12

### Cellular Production of Target Polypeptides

Nucleic acids are cloned into DNA plasmids having phage recombination cites and target polypeptides are expressed therefrom in a variety of host cells. Alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of target polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the target polypeptide or polypeptide variants. These expression vectors are often transfected, for example, into murine-transformed a adipocyte cell line 3T3-L1, (ATCC), human embryonic kidney cell line 293, and rat cardiomyocyte cell line H9C2.

### Example 13

### Haplotype Analysis of the KIAA0861 Locus

rs6804951 and rs2001449 are significant at the allele and genotype levels (P<0.05). Moderate LD is observed for markers rs3732602 and rs2293203 (r̂2=0.646). Chi-squared tests indicate that haplotypes are significantly associated with breast cancer. Cell-specific chi-square values indicate that TTTTG and CTTTC haplotypes are contributors to this relationship. Odds ratios and score tests indicate that individuals carrying the TTTG are less likely to have breast cancer, while individuals with CTTTC are at elevated risk for the disease. Moreover, the odds ratio estimated for the CGTTC indicates more than a two-fold risk of disease among its carriers, although this result must be interpreted with great caution due to the low observed frequency in the population.

Summary Statistics of Alleles and Genotypes

SNP Locations

| SNP.ID | Type | Location |
|---|---|---|
| rs6804951 | Proximal | 184327431 |
| rs7639705 | Proximal | 184330963 |
| rs3732602 | Proximal | 184408945 |
| rs2293203 | Proximal | 184419992 |
| rs2001449 | Incident | 184429569 |

Allele by GYNGroup

| | | Case | Control | Test |
|---|---|---|---|---|
| | N | (N = 544) | (N = 552) | Statistic |
| rs6804951:T | 1064 | 5% (24) | 9% (46) | Chi-square = 6.71 d.f. = 1 P = 0.00958 |
| rs7639705:T | 1086 | 80% (434) | 81% (441) | Chi-square = 0.03 d.f. = 1 P = 0.868 |
| rs3732602:T | 1074 | 99% (532) | 99% (532) | Chi-square = 0.4 d.f. = 1 P = 0.529 |
| rs2293203:T | 1088 | 99% (536) | 99% (538) | Chi-square = 0.27 d.f. = 1 P = 0.6 |
| \|rs200144:C | 1084 | 30% (161) | 22% (119) | Chi-square = 8.49 d.f. P = 0.00356 |

Genotype by GYNGroup

| | | Case | | Control | | Test |
|---|---|---|---|---|---|---|
| | N | (N = 272) | | (N = 276) | | Statistic |
| rs6804951:CC | 532 | 91% | (238) | 83% | (225) | Chi-square = 7.13 d.f. = 2 P = 0.0283 |
| CT | | 9% | (24) | 16% | (44) | |
| TT | | 0% | (0) | 0% | (1) | |
| rs7639705:GG | 543 | 3% | (9) | 5% | (14) | Chi-square = 2.03 d.f. = 2 P = 0.362 |
| GT | | 33% | (88) | 28% | (77) | |
| TT | | 64% | (173) | 67% | (182) | |
| rs3732602:TT | 537 | 99% | (264) | 98% | (263) | Chi-square = 0.4 d.f. = 1 P = 0.527 |
| rs2293203:TT | 544 | 98% | (265) | 97% | (265) | Chi-square = 0.28 d.f. = 1 P = 0.598 |
| rs2001449:GG | 542 | 47% | (128) | 60% | (162) | Chi-square = 9.29 d.f. = 2 P = 0.00961 |
| GC | | 46% | (125) | 37% | (99) | |
| CC | | 7% | (18) | 4% | (10) | |

Genotype QC: Test of Hardy-Weinberg Equilibrium

Cases

| | A. freq | D | ChiSq | Pvalue |
|---|---|---|---|---|
| rs6804951 | 0.936 | −0.002280 | 0.7870 | 0.3750 |
| rs7639705 | 0.807 | 0.004790 | 0.5150 | 0.4730 |
| rs3732602 | 0.990 | −0.000101 | 0.0565 | 0.8120 |
| rs2293203 | 0.987 | −0.000164 | 0.0921 | 0.7620 |
| rs2001449 | 0.744 | −0.014500 | 3.1400 | 0.0763 |

Controls

| | A. freq | D | ChiSq | Pvalue |
|---|---|---|---|---|
| rs6804951 | 0.916 | −0.003400 | 0.5350 | 0.465 |
| rs7639705 | 0.808 | −0.014400 | 2.3600 | 0.124 |
| rs3732602 | 0.989 | 0.000120 | 0.0336 | 0.855 |
| rs2293203 | 0.985 | −0.000213 | 0.0601 | 0.806 |
| rs2001449 | 0.783 | −0.010700 | 1.0800 | 0.299 |

Summary Statistics: Linkage Disequilibrium

PHASE Haplotype Frequencies

| | H.freq | H.relfreq |
|---|---|---|
| CGTTC | 13 | 0.012 |
| CGTTG | 191 | 0.175 |
| CTCAG | 10 | 0.009 |
| CTCTG | 1 | 0.001 |
| CTTAG | 4 | 0.004 |
| CTTTC | 265 | 0.243 |
| CTTTG | 538 | 0.493 |
| TGTTG | 7 | 0.006 |
| TTTTC | 2 | 0.002 |
| TTTTG | 61 | 0.056 |

Linkage Disequilibrium Between Markers

r\hat{ }2

| | rs6804951 | rs7639705 | rs3732602 | rs2293203 | rs2001449 |
|---|---|---|---|---|---|
| rs6804951 | 1.000000 | 0.00382 | 0.000697 | 0.00089 | 0.01860 |
| rs7639705 | 0.003820 | 1.00000 | 0.002440 | 0.00311 | 0.04770 |
| rs3732602 | 0.000697 | 0.00244 | 1.000000 | 0.64600 | 0.00351 |
| rs2293203 | 0.000890 | 0.00311 | 0.646000 | 1.00000 | 0.00448 |
| rs2001449 | 0.018600 | 0.04770 | 0.003510 | 0.00448 | 1.00000 |

D′

| | rs6804951 | rs7639705 | rs3732602 | rs2293203 | rs2001449 |
|---|---|---|---|---|---|
| rs6804951 | 1.0000 | 0.116 | 0.0685 | 0.0685 | 0.306 |
| rs7639705 | 0.1160 | 1.000 | 0.2400 | 0.2400 | 0.262 |
| rs3732602 | 0.0685 | 0.240 | 1.0000 | 0.9080 | 0.345 |
| rs2293203 | 0.0685 | 0.240 | 0.9080 | 1.0000 | 0.345 |
| rs2001449 | 0.3060 | 0.262 | 0.3450 | 0.3450 | 1.000 |

P-value

| | rs6804951 | rs7639705 | rs3732602 | rs2293203 | rs2001449 |
|---|---|---|---|---|---|
| rs6804951 | 1.00e+00 | 4.12e−02 | 0.3830 | 0.3240 | 6.40e−06 |
| rs7639705 | 4.12e−02 | 1.00e+00 | 0.1030 | 0.0653 | 5.41e−13 |
| rs3732602 | 3.83e−01 | 1.03e−01 | 1.0000 | 0.0000 | 5.03e−02 |
| rs2293203 | 3.24e−01 | 6.53e−02 | 0.0000 | 1.0000 | 2.70e−02 |
| rs2001449 | 6.40e−06 | 5.41e−13 | 0.0503 | 0.0270 | 1.00e+00 |

Haplotype by GYNGroup

PHASE Haplotypes (All)

| | Case | Case (%) | Case. X\hat{ }2 | Control | Control (%) | Control. X\hat{ }2 | OR | In. OR |
|---|---|---|---|---|---|---|---|---|
| TTTTG | 20 | 1.83 | 3.55 | 41 | 3.75 | 3.53 | 0.4782 | −0.7377 |
| CTCAG | 4 | 0.37 | 0.19 | 6 | 0.55 | 0.19 | 0.6654 | −0.4074 |
| TGTTG | 3 | 0.27 | 0.07 | 4 | 0.37 | 0.07 | 0.7493 | −0.2886 |
| CTTTG | 259 | 23.72 | 0.30 | 279 | 25.55 | 0.30 | 0.9060 | −0.987 |
| CGTTG | 94 | 8.61 | 0.01 | 97 | 8.88 | 0.01 | 0.9662 | −0.0344 |
| CTTAG | 2 | 0.18 | 0.00 | 2 | 0.18 | 0.00 | 1.0000 | 0.0000 |
| TTTTC | 1 | 0.09 | 0.00 | 1 | 0.09 | 0.00 | 1.0000 | 0.0000 |
| CTTTC | 151 | 13.83 | 2.73 | 114 | 10.44 | 2.71 | 1.3766 | 0.3196 |
| CGTTC | 9 | 0.82 | 0.98 | 4 | 0.37 | 0.98 | 2.2604 | 0.8155 |
| CTCTG | 1 | 0.09 | 0.51 | 0 | 0.00 | 0.50 | Inf | Inf |

Pearson Chi-squared Test = 16.6377,
DF = 9,
P-value = 0.0547

PHASE Haplotypes (Low Frequency Removed)

| | Case | Case (%) | Case. X\hat{ }2 | Control | Control (%) | Control. X\hat{ }2 | OR | In. OR |
|---|---|---|---|---|---|---|---|---|
| TTTTG | 20 | 1.86 | 3.55 | 41 | 3.80 | 3.52 | 0.4781 | −0.7379 |
| CTCAG | 4 | 0.37 | 0.19 | 6 | 0.56 | 0.19 | 0.6654 | −0.4074 |
| CTTTG | 259 | 24.03 | 0.30 | 279 | 25.88 | 0.30 | 0.9056 | −0.0992 |
| CGTTG | 94 | 8.72 | 0.01 | 97 | 9.00 | 0.01 | 0.9661 | −0.0345 |
| CTTTC | 151 | 14.01 | 2.73 | 114 | 10.58 | 2.71 | 1.3774 | 0.3202 |
| CGTTC | 9 | 0.83 | 0.98 | 4 | 0.37 | 0.98 | 2.2605 | 0.8156 |

Pearson Chi-squared Test = 15.4946,
DF = 5,
P-value = 0.008445

haplo.score Haplotypes

| | Hap. Freq | Score P. | X\hat{ }2 | P. Sim |
|---|---|---|---|---|
| TTTTG | 0.0529 | −2.1206 | 0.0340 | 0.0342 |
| TGTTG | 0.0101 | −2.0668 | 0.0388 | 0.0236 |
| CTCAG | 0.0073 | −1.2914 | 0.1966 | 0.2902 |
| CTTTG | 0.5221 | −1.2275 | 0.2196 | 0.2195 |
| CGTTG | 0.1448 | −0.1441 | 0.8854 | 0.8834 |
| CTTTC | 0.2267 | 2.3422 | 0.0192 | 0.0192 |
| CGTTC | 0.0307 | 2.6994 | 0.0069 | 0.0050 |

Global Score = 20.343, DF = 7, Global P. X\hat{ }2 = 0.0049, Global P. Sim = 0.0022

### Example 14

### Haplotype Analysis of the NUMA1 Locus

All markers noted below except 2276396 are associated with breast cancer at the allele level (P<0.05). Marker 675185 does not maintain this relationship at the genotype level. Strong LD is observed across the entire region but is particular strong between and among 1894003, 675185, 673478, and 615000. Pearson chi-squared statistics suggest that haplotypes are significantly associated with breast cancer. Haplotype TTCTC contributes the most to this relationship. Odds ratios and score statistics indicate that individuals with haplotype TTCTC are 2.6 times more likely to have breast cancer than individuals with other haplotypes.

Statistics

Chi-squared statistics are estimated to assess whether 1) alleles and genotypes are associated with breast cancer status and 2) marker genotype frequencies deviate significantly from Hardy-Weinberg equilibrium (HWE). Haplotype frequencies and relative frequencies are estimated, as well as several statistics (r², D′, and p-value) that gauge the extent and stability of linkage disequilibrium between markers in each region. Chi-squared statistics and score tests are estimated to determine whether reconstructed haplotypes are significantly associated with breast cancer status (P<0.05). P-values are estimated for 1) the full set of reconstructed haplotypes and 2) a reduced set that excludes haplotypes with observed frequencies less than 10. Results are presented by chromosome order.

### Results

Summary Statistics: Alleles and Genotypes

SNP Locations

| SNP. ID | Type | Location |
|---|---|---|
| 1894003 | Proximal | 71972974 |
| 675185 | Proximal | 71998270 |
| 673478 | Incident | 72021802 |
| 615000 | Proximal | 72025650 |
| 2276396 | Proximal | 72046603 |

Allele by GYNGroup

| | | Case | | Control | | Test |
|---|---|---|---|---|---|---|
| | N | (N = 510) | | (N-538) | | Statistic |
| 1894003:C | 1026 | 91% | (450) | 96% | (510) | Chi-square = 6.95 d.f. = 1 P = 0.00838 |
| 675185:G | 1010 | 92% | (451) | 95% | (498) | Chi-square = 3.96 d.f. = 1 P = 0.0466 |
| 673478:C | 1022 | 8% | (41) | 5% | (25) | Chi-square = 5.68 d.f. = 1 P = 0.0171 |
| 615000:G | 1010 | 92% | (434) | 96% | (513) | Chi-square = 7.4 d.f. = 1 P = 0.00652 |
| 2276396:C | 1028 | 97% | (478) | 98% | (523) | Chi-square = 0.18 d.f. = 1 P = 0.674 |

Genotype by GYNGroup

| | | Case | | Control | | Test |
|---|---|---|---|---|---|---|
| | N | (N = 255) | | (N = 269) | | Statistic |
| 1894003:TT | 513 | 1% | (3) | 0% | (0) | Chi-square = 7.43 d.f. = 2 P = 0.0243 |
| TC | | 15% | (36) | 9% | (24) | |
| CC | | 84% | (207) | 91% | (243) | |
| 675185:TT | 505 | 0% | (1) | 0% | (0) | Chi-square = 4.37 d.f. = 2 P = 0.112 |
| TG | | 14% | (35) | 9% | (24) | |
| GG | | 85% | (208) | 91% | (237) | |
| 673478:TT | 511 | 84% | (207) | 91% | (241) | Chi-square = 6.39 d.f. = 2 P = 0.0409 |
| TC | | 14% | (35) | 9% | (25) | |
| CC | | 1% | (3) | 0% | (0) | |
| 615000:TT | 505 | 1% | (3) | 0% | (0) | Chi-square = 7.8 d.f. = 2 P = 0.0202 |
| TG | | 14% | (34) | 9% | (23) | |
| GG | | 84% | (200) | 91% | (245) | |
| 2276396:CC | 514 | 4% | (232) | 95% | (255) | Chi-square = 0.18 d.f. = 1 P = 0.67 |

### Genotype QC: Test of Hardy-Weinberg Proportions

All

| | A. freq | D | ChiSq | Pvalue |
|---|---|---|---|---|
| 1894003 | 0.935 | 0.00159 | 0.350 | 0.554 |
| 675185 | 0.935 | 0.00159 | 0.350 | 0.554 |
| 673478 | 0.935 | 0.00159 | 0.350 | 0.554 |
| 615000 | 0.937 | 0.00184 | 0.495 | 0.482 |
| 2276396 | 0.974 | −0.00069 | 0.374 | 0.541 |

Control

| | A. freq | D | ChiSq | Pvalue |
|---|---|---|---|---|
| 1894003 | 0.953 | −0.002190 | 0.644 | 0.422 |
| 675185 | 0.953 | −0.002190 | 0.644 | 0.422 |
| 673478 | 0.953 | −0.002190 | 0.644 | 0.422 |
| 615000 | 0.957 | −0.001860 | 0.541 | 0.462 |
| 2276396 | 0.976 | −0.000593 | 0.166 | 0.683 |

### Summary Statistics: Linkage Disequilibrium

Haplotype Frequencies

| | H.freq | H.relfreq |
|---|---|---|
| CGTGC | 961 | 0.935 |
| TTCGC | 1 | 0.001 |
| TTCGG | 1 | 0.001 |
| TTCTC | 39 | 0.038 |
| TTCTG | 26 | 0.025 |

### Linkage Disequilibrium Between Markers

r²

| | 1894003 | 675185 | GP11.079035103 | 615000 | 2276396 |
|---|---|---|---|---|---|
| 1894003 | 1.000 | 1.000 | 1.000 | 0.968 | 0.387 |
| 675185 | 1.000 | 1.000 | 1.000 | 0.968 | 0.387 |
| 673478 | 1.000 | 1.000 | 1.000 | 0.968 | 0.387 |
| 615000 | 0.968 | 0.968 | 0.968 | 1.000 | 0.369 |
| 2276396 | 0.387 | 0.387 | 0.387 | 0.369 | 1.000 |

D′

| | 1894003 | 675185 | GP11.079035103 | 615000 | 2276396 |
|---|---|---|---|---|---|
| 1894003 | 1 | 1 | 1 | 1.00 | 1.00 |
| 675185 | 1 | 1 | 1 | 1.00 | 1.00 |
| 673478 | 1 | 1 | 1 | 1.00 | 1.00 |
| 615000 | 1 | 1 | 1 | 1.00 | 0.96 |
| 2276396 | 1 | 1 | 1 | 0.96 | 1.00 |

P-value

| | | | GP11.- | | |
|---|---|---|---|---|---|
| X | 1894003 | 675185 | 079035103 | 615000 | 2276396 |
| 1894003 | 1 | 0 | 0 | 0 | 0 |
| 675185 | 0 | 1 | 0 | 0 | 0 |
| GP11.- | 0 | 0 | 1 | 0 | 0 |
| 079035103 | | | | | |
| 615000 | 0 | 0 | 0 | 1 | 0 |
| 2276396 | 0 | 0 | 0 | 0 | 1 |

### Haplotype by GYNGroup

PHASE Haplotypes (All)

| | | | | | | | |
|---|---|---|---|---|---|---|---|
| Case | Case (%) | Case.X\hat{ }2 | Control | Control (%) | Control.X\hat{ }2 | OR | ln.OR |

| | | | | | | | | |
|---|---|---|---|---|---|---|---|---|
| TTCGC | 0 | 0.00 | 0.48 | 1 | 0.10 | 0.44 | 0.0000 | −Inf |
| TTCGG | 0 | 0.00 | 0.48 | 1 | 0.10 | 0.44 | 0.0000 | −Inf |
| CGTGC | 452 | 43.97 | 0.21 | 509 | 49.51 | 0.19 | 0.8001 | −0.2230 |
| TTCTG | 14 | 1.36 | 0.18 | 12 | 1.17 | 0.17 | 1.1690 | 0.1561 |
| TTCTC | 28 | 2.72 | 4.57 | 11 | 1.07 | 4.23 | 2.5887 | 0.9512 |

Pearson Chi-squared Test = 11.4058,
DF = 4,
P-value = 0.02236
Permutation Test P-value = 0.14

PHASE Haplotypes (Low Frequency Excluded)

| | | | | | | | |
|---|---|---|---|---|---|---|---|
| Case | Case (%) | Case.X\hat{ }2 | Control | Control (%) | Control.X\hat{ }2 | OR | ln.OR |

| | | | | | | | | |
|---|---|---|---|---|---|---|---|---|
| CGTGC | 452 | 44.05 | 0.25 | 509 | 49.61 | 0.23 | 0.7998 | −0.2234 |
| TTCTG | 14 | 1.36 | 0.18 | 12 | 1.17 | 0.16 | 1.1690 | 0.1561 |
| TTCTC | 28 | 2.73 | 4.53 | 11 | 1.07 | 4.21 | 2.5888 | 0.9512 |

Pearson Chi-squared Test = 9.5506,
DF = 2,
P-value = 0.008435

haplo.score Haplotypes

| | Hap.Freq | Score | P.X\hat{ }2 | P.Sim |
|---|---|---|---|---|
| CGTGC | 0.9410 | −2.0316 | 0.0422 | 0.0531 |
| TTCTG | 0.0248 | 0.3232 | 0.7465 | 0.8344 |
| TTCTC | 0.0321 | 2.6973 | 0.0070 | 0.0093 |

Global Score = 9.1386,
DF = 3,
Global P.X\hat{ }2 = 0.0275,
Global P.Sim = 0.0212

### Example 15

### GALE Region Proximal SNPs

It has been discovered that a polymorphic variation (rs4237) in the HT014/LOC148902/LYPLA2/GALE region is associated with the occurrence of breast cancer (see Examples 1 and 2). Subsequently, SNPs proximal to the incident SNP (rs4237) were identified and allelotyped in breast cancer sample sets and control sample sets as described in Examples 1 and 2. Approximately 175 allelic variants located within the HT014/LOC148902/LYPLA2/GALE region were identified and allelotyped. The polymorphic variants are set forth in Table 37. The chromosome position provided in column four of Table 37 is based on Genome “Build 34” of NCBI's GenBank.

TABLE 37

| | Position in | | | | | |
|---|---|---|---|---|---|---|
| | SEQ ID | | Chromosome | Allele | Genome | Deduced |
| dbSNP rs# | NO: 5 | Chromosome | Position | Variants | Letter | Iupac |
| 627451 | 215 | 1 | 23536215 | g/a | g | R |
| 12082331 | 385 | 1 | 23536385 | g/c | g | S |
| 2267960 | 2019 | 1 | 23538019 | t/c | t | Y |
| 7519640 | 3112 | 1 | 23539112 | c/g | g | S |
| 12086741 | 3437 | 1 | 23539437 | g/a | g | R |
| 3934189 | 4326 | 1 | 23540326 | t/c | t | Y |
| 12135415 | 4523 | 1 | 23540523 | a/g | a | R |
| 12088267 | 4645 | 1 | 23540645 | a/g | g | R |
| 12084665 | 4646 | 1 | 23540646 | c/t | t | Y |
| 550850 | 5830 | 1 | 23541830 | t/c | c | Y |
| 557227 | 6556 | 1 | 23542556 | g/t | g | K |
| 12137502 | 6568 | 1 | 23542568 | t/a | a | W |
| 12118858 | 6570 | 1 | 23542570 | c/t | c | Y |
| 2294495 | 9351 | 1 | 23545351 | c/t | c | Y |
| 12401650 | 10874 | 1 | 23546874 | c/t | c | Y |
| 2235541 | 10926 | 1 | 23546926 | c/t | c | Y |
| 520713 | 11462 | 1 | 23547462 | g/a | g | R |
| 550252 | 12415 | 1 | 23548415 | t/c | c | Y |
| HT014-AA | 12668 | 1 | 23548668 | a/g | g | R |
| 12093683 | 13886 | 1 | 23549886 | g/c | g | S |
| 10917421 | 15005 | 1 | 23551005 | g/a | g | R |
| 2744873 | 15387 | 1 | 23551387 | a/t | t | W |
| 12091764 | 15639 | 1 | 23551639 | g/a | a | R |
| HT014-AB | 15761 | 1 | 23551761 | a/t | t | W |
| 2076345 | 16969 | 1 | 23552969 | c/t | c | Y |
| 2076346 | 17124 | 1 | 23553124 | t/c | t | Y |
| 2473379 | 17369 | 1 | 23553369 | a/g | a | R |
| 6679300 | 17523 | 1 | 23553523 | c/g | g | S |
| 11811395 | 18158 | 1 | 23554158 | t/a | t | W |
| 8179467 | 18220 | 1 | 23554220 | t/a | a | W |
| 8179468 | 18224 | 1 | 23554224 | t/a | a | W |
| 10917423 | 18538 | 1 | 23554538 | a/g | g | R |
| 7536699 | 18810 | 1 | 23554810 | g/c | g | S |
| 3215497 | 20282 | 1 | 23556282 | —/t | t | N |
| 1000213 | 20495 | 1 | 23556495 | c/a | t | K |
| 1000212 | 20633 | 1 | 23556633 | c/t | g | R |
| 1000211 | 20659 | 1 | 23556659 | g/a | c | Y |
| 1045017 | 21603 | 1 | 23557603 | a/g | g | R |
| 12059520 | 24075 | 1 | 23560075 | c/g | g | S |
| 2143118 | 24191 | 1 | 23560191 | c/t | c | Y |
| 12126804 | 25010 | 1 | 23561010 | t/c | c | Y |
| 533276 | 25124 | 1 | 23561124 | c/t | c | Y |
| 12402174 | 25145 | 1 | 23561145 | g/c | g | S |
| 2864110 | 25222 | 1 | 23561222 | c/a | t | K |
| 11581873 | 25288 | 1 | 23561288 | t/c | c | Y |
| 12062449 | 26080 | 1 | 23562080 | g/a | g | R |
| 12033190 | 26556 | 1 | 23562556 | c/t | c | Y |
| 10157566 | 27416 | 1 | 23563416 | t/c | t | Y |
| 486746 | 27432 | 1 | 23563432 | c/g | c | S |
| 11802707 | 29841 | 1 | 23565841 | a/g | g | R |
| 6668550 | 29955 | 1 | 23565955 | c/a | a | M |
| 7410946 | 30160 | 1 | 23566160 | a/g | g | R |
| 7410970 | 30294 | 1 | 23566294 | g/c | g | S |
| 6657558 | 31039 | 1 | 23567039 | c/g | g | S |
| 2143119 | 32071 | 1 | 23568071 | t/c | c | Y |
| 7514394 | 33698 | 1 | 23569698 | t/c | c | Y |
| 11591202 | 35555 | 1 | 23571555 | a/c | a | M |
| 6673991 | 36658 | 1 | 23572658 | a/g | a | R |
| HT014-AC | 37720 | 1 | 23573720 | g/c | c | S |
| 12034848 | 38304 | 1 | 23574304 | c/t | c | Y |
| 2502984 | 39008 | 1 | 23575008 | t/c | t | Y |
| 3831910 | 39985 | 1 | 23575985 | a/— | a | N |
| 11379444 | 39989 | 1 | 23575989 | —/t | t | N |
| 725119 | 40683 | 1 | 23576683 | t/a | a | W |
| 10917425 | 42158 | 1 | 23578158 | t/c | c | Y |
| 12077788 | 44369 | 1 | 23580369 | c/g | c | S |
| 12077830 | 44503 | 1 | 23580503 | c/g | c | S |
| 2473380 | 45382 | 1 | 23581382 | c/t | t | Y |
| 2473381 | 46171 | 1 | 23582171 | a/c | a | M |
| 11545132 | 47276 | 1 | 23583276 | g/t | g | K |
| 1046924 | 47299 | 1 | 23583299 | t/c | t | Y |
| 4237 | 47604 | 1 | 23583604 | a/g | g | R |
| 4375317 | 48462 | 1 | 23584462 | g/a | g | R |
| 11584220 | 50786 | 1 | 23586786 | a/c | c | M |
| 2232975 | 51057 | 1 | 23587057 | g/t | g | K |
| 2502985 | 51310 | 1 | 23587310 | g/a | g | R |
| 3835423 | 51996 | 1 | 23587996 | c/— | g | N |
| 2232976 | 52546 | 1 | 23588546 | t/g | t | K |
| 2232977 | 52602 | 1 | 23588602 | c/t | c | Y |
| 2232978 | 52725 | 1 | 23588725 | g/a | g | R |
| 2232979 | 52858 | 1 | 23588858 | a/c | c | M |
| 2232980 | 52935 | 1 | 23588935 | t/g | g | K |
| 2232981 | 52948 | 1 | 23588948 | c/t | c | Y |
| 2232982 | 53102 | 1 | 23589102 | t/c | c | Y |
| 2232983 | 54160 | 1 | 23590160 | c/a | c | M |
| 2232984 | 54794 | 1 | 23590794 | c/t | c | Y |
| 2232985 | 54798 | 1 | 23590798 | t/c | c | Y |
| 2232986 | 54853 | 1 | 23590853 | t/c | c | Y |
| 2745 | 55422 | 1 | 23591422 | a/g | c | Y |
| 2744 | 55460 | 1 | 23591460 | c/g | g | S |
| 11546127 | 55645 | 1 | 23591645 | g/t | g | K |
| 1049988 | 55660 | 1 | 23591660 | t/c | g | R |
| 1803613 | 55729 | 1 | 23591729 | c/t | g | R |
| 1803612 | 55736 | 1 | 23591736 | t/g | c | M |
| HT014-AD | 55821 | 1 | 23591821 | t/c | c | Y |
| 3209973 | 55970 | 1 | 23591970 | t/g | c | M |
| 3180545 | 56150 | 1 | 23592150 | c/a | c | M |
| 3180383 | 56167 | 1 | 23592167 | a/g | g | R |
| 3177814 | 56171 | 1 | 23592171 | g/t | g | K |
| 3180546 | 56210 | 1 | 23592210 | g/t | t | K |
| 3180547 | 56213 | 1 | 23592213 | a/c | c | M |
| 11805398 | 56259 | 1 | 23592259 | t/g | g | K |
| 760941 | 56461 | 1 | 23592461 | t/c | c | Y |
| 3177813 | 56480 | 1 | 23592480 | t/c | c | Y |
| 3180548 | 56486 | 1 | 23592486 | a/g | g | R |
| 3179864 | 56499 | 1 | 23592499 | a/g | g | R |
| 3177812 | 56510 | 1 | 23592510 | t/c | c | Y |
| 3179863 | 56543 | 1 | 23592543 | g/t | g | K |
| 6692104 | 56827 | 1 | 23592827 | g/c | g | S |
| 3180549 | 56846 | 1 | 23592846 | g/c | c | S |
| 3179862 | 56859 | 1 | 23592859 | c/a | c | M |
| 3177811 | 56870 | 1 | 23592870 | g/c | c | S |
| 3177810 | 56890 | 1 | 23592890 | c/a | c | M |
| 3180382 | 56907 | 1 | 23592907 | a/c | c | M |
| 11546126 | 57767 | 1 | 23593767 | c/t | a | R |
| 7551384 | 58774 | 1 | 23594774 | c/g | g | S |
| 12141511 | 59444 | 1 | 23595444 | t/c | c | Y |
| 2473382 | 59452 | 1 | 23595452 | g/a | g | R |
| 12038166 | 59753 | 1 | 23595753 | a/g | g | R |
| 6666908 | 60766 | 1 | 23596766 | c/a | c | M |
| 12041159 | 61135 | 1 | 23597135 | g/c | c | S |
| 12031592 | 61339 | 1 | 23597339 | a/c | a | M |
| 1062598 | 61922 | 1 | 23597922 | c/t | g | R |
| 11714 | 61942 | 1 | 23597942 | c/t | c | Y |
| 3203593 | 61958 | 1 | 23597958 | a/g | c | Y |
| 1042436 | 62068 | 1 | 23598068 | t/c | g | R |
| 2076343 | 62601 | 1 | 23598601 | a/g | a | R |
| 12131539 | 62661 | 1 | 23598661 | g/a | g | R |
| 11583935 | 62882 | 1 | 23598882 | t/g | g | K |
| 12140263 | 63949 | 1 | 23599949 | a/c | c | M |
| 3835225 | 64732 | 1 | 23600732 | —/ag | g | N |
| 2473374 | 65145 | 1 | 23601145 | c/t | t | Y |
| 2256179 | 65780 | 1 | 23601780 | a/g | g | R |
| 974698 | 66260 | 1 | 23602260 | c/t | c | Y |
| 1018396 | 67087 | 1 | 23603087 | c/t | g | R |
| 719399 | 67937 | 1 | 23603937 | g/a | c | Y |
| 719400 | 68196 | 1 | 23604196 | g/a | t | Y |
| 2502986 | 68767 | 1 | 23604767 | g/t | g | K |
| 6679378 | 69482 | 1 | 23605482 | t/c | c | Y |
| 2473375 | 70020 | 1 | 23606020 | c/t | c | Y |
| 2473376 | 72759 | 1 | 23608759 | a/g | g | R |
| 12136455 | 72911 | 1 | 23608911 | g/c | g | S |
| 2502987 | 73331 | 1 | 23609331 | c/a | a | M |
| 11551767 | 74250 | 1 | 23610250 | c/t | g | R |
| 11591205 | 74486 | 1 | 23610486 | t/c | c | Y |
| 12083298 | 74921 | 1 | 23610921 | a/t | a | W |
| 12138604 | 75065 | 1 | 23611065 | g/a | g | R |
| 12136042 | 75077 | 1 | 23611077 | c/t | t | Y |
| 12138606 | 75078 | 1 | 23611078 | g/a | g | R |
| 12136045 | 75096 | 1 | 23611096 | c/t | t | Y |
| 12116596 | 75108 | 1 | 23611108 | c/g | c | S |
| 12138610 | 75121 | 1 | 23611121 | a/g | g | R |
| 12136069 | 75136 | 1 | 23611136 | t/c | t | Y |
| 12116617 | 75150 | 1 | 23611150 | c/t | c | Y |
| 7540056 | 75594 | 1 | 23611594 | g/a | a | R |
| 12044452 | 77120 | 1 | 23613120 | t/g | g | K |
| 2076344 | 77407 | 1 | 23613407 | g/a | a | R |
| 11588187 | 78522 | 1 | 23614522 | t/a | t | W |
| 6696932 | 78656 | 1 | 23614656 | t/a | a | W |
| 12025531 | 78902 | 1 | 23614902 | t/c | c | Y |
| 6682888 | 78911 | 1 | 23614911 | g/a | g | R |
| 2982390 | 81305 | 1 | 23617305 | g/c | g | S |
| 2179394 | 81717 | 1 | 23617717 | a/t | a | W |
| 2473377 | 81735 | 1 | 23617735 | c/t | t | Y |
| 2179395 | 81840 | 1 | 23617840 | t/c | t | Y |
| 2502978 | 82511 | 1 | 23618511 | c/t | c | Y |
| 2502979 | 82790 | 1 | 23618790 | a/c | c | M |
| 6698535 | 83323 | 1 | 23619323 | t/a | t | W |
| 2502980 | 83464 | 1 | 23619464 | t/c | c | Y |
| 10554242 | 83916-83920 | 1 | 23619916\hat{ }23619919 | cact/— | cact | NNNN |
| 6697805 | 85471 | 1 | 23621471 | a/c | c | M |
| 6424114 | 85688 | 1 | 23621688 | c/a | c | M |
| 5773058 | 86196 | 1 | 23622196 | —/tt | g | N |
| 4649114 | 86323 | 1 | 23622323 | t/g | g | K |
| 6424115 | 86706 | 1 | 23622706 | a/g | a | R |

### Assay for Verifying and Allelotyping SNPs

The methods used to verify and allelotype the proximal SNPs of Table 37 are the same methods described in Examples 1 and 2 herein. The PCR primers and extend primers used in these assays are provided in Table 38 (SEQ ID NOS 2203-2552, respectively, in order of appearance) and Table 39 (SEQ ID NOS 2553-2727, respectively, in order of appearance), respectively.

TABLE 38

| | | |
|---|---|---|
| dbSNP rs# | Forward PCR primer | Reverse PCR primer |

| | | |
|---|---|---|
| 627451 | ACGTTGGATGATCTTCTGACCTCGTGATCC | ACGTTGGATGCAACAGAGTGAGACCCTGTC |
| 12082331 | ACGTTGGATGGGGTTCATGCCGTTCTCCTG | ACGTTGGATGAAATTAGCCGGGTGTAGTGG |
| 2267960 | ACGTTGGATGCACCTAGCATCATCTAGACC | ACGTTGGATGTTAGGGTACCAAGTGGATGG |
| 7519640 | ACGTTGGATGGCGTTGGCATTTATAAGGCC | ACGTTGGATGTCTCCAGGGTCTACTTCAAG |
| 12086741 | ACGTTGGATGAACTTGGAGCGCCGACTCCG | ACGTTGGATGATGCACGGAGGGCGGAGCT |
| 3934189 | ACGTTGGATGACCAGAGAAGTGAGTGACAG | ACGTTGGATGTTTTGTTGTTTATTGTAC |
| 12135415 | ACGTTGGATGATCTCGGCTCACTGCAACCT | ACGTTGGATGGCGCCTACAATACCAGCTAC |
| 12088267 | ACGTTGGATGTAGTAGAGACAGGGTTTCAC | ACGTTGGATGTGACGGAGAATGCCAAACAC |
| 12084665 | ACGTTGGATGTAGTAGAGACAGGGTTTCAC | ACGTTGGATGTGACGGAGAATGCCAAACAC |
| 550850 | ACGTTGGATGTCTCCCTTAAGTGAGCCAAG | ACGTTGGATGTTCCACACCTGTGTTCATGG |
| 557227 | ACGTTGGATGTGTAACACAGGACGAGGATC | ACGTTGGATGATGGCACCATTGCACTCCAG |
| 12137502 | ACGTTGGATGAGCCTGGACAACAAGAGCAA | ACGTTGGATGTGTAACACAGGACGAGGATC |
| 12118858 | ACGTTGGATGAGCCTGGACAACAAGAGCAA | ACGTTGGATGTGTAACACAGGACGAGGATC |
| 2294495 | ACGTTGGATGGGAATTAGATGGTTATAATGG | ACGTTGGATGCACCACTTTAACTAAAGTAC |
| 12401650 | ACGTTGGATGAGAAGAGCAATTCCCGAAAG | ACGTTGGATGTCCAGGTTTCTTGGTAGTCC |
| 2235541 | ACGTTGGATGGGGACTACCAAGAAACCTGG | ACGTTGGATGTCCACGAGCTCCGAGAGTTTCCTATG |
| 520713 | ACGTTGGATGAGGGAGGCAAACACTTCTTC | ACGTTGGATGACAATGCAAGGGAGAAACCG |
| 550252 | ACGTTGGATGAGGCAGTGGACGGTAATTGG | ACGTTGGATGTCCACGCTGCAGGTGCCTGATGTGTT |
| HT014-AA | ACGTTGGATGTTCTAAACCAACCAGGCGTG | ACGTTGGATGTTACCTAAGGTGATCTGCCC |
| 12093683 | ACGTTGGATGTAAGCTCCTGTAATCCCAGC | ACGTTGGATGAGGCTGATCTCGAACTCTTG |
| 10917421 | ACGTTGGATGATATTAGCCAGCTGTGGTGG | ACGTTGGATGACTGCATCCTTGAACTCCTG |
| 2744873 | ACGTTGGATGACCTAGGAGTGACCAGTTTC | ACGTTGGATGGGGAGTTATTTTTTTCCTAG |
| 12091764 | ACGTTGGATGTGGGTTCAAGCGATTCTCAC | ACGTTGGATGTAAAATAGCTGGGCGTGGTG |
| HT014-AB | ACGTTGGATGGAAACCAGAACCAGGACATC | ACGTTGGATGACAGAGCAAGACTCTGTCTC |
| 2076345 | ACGTTGGATGATGGGAAGCAGCCATGCTTC | ACGTTGGATGTCCACGTGGTGCTTGGGCCATCATAG |
| 2076346 | ACGTTGGATGGCAGTCCACAAAATGTAATAC | ACGTTGGATGAAGTAACTTGGAGTTCCTGC |
| 2473379 | ACGTTGGATGTAATCCCAGCACTTTGGCAG | ACGTTGGATGTCTCATTATGTTGTCCAGGC |
| 6679300 | ACGTTGGATGTTTTTGAGATGGAGTCTCGC | ACGTTGGATGAATGGCATGAACCTGGAAGG |
| 11811395 | ACGTTGGATGAATGACAGGCCCTCTTACTG | ACGTTGGATGAGAGTGAGACTCTGTCTCCA |
| 8179467 | ACGTTGGATGAATGACAGGCCCTCTTACTG | ACGTTGGATGAGAGTGAGACTCTGTCTCCA |
| 8179468 | ACGTTGGATGTATATCTAATTGGGTCTTCC | ACGTTGGATGAGAGTGAGACTCTGTCTCCA |
| 10917423 | ACGTTGGATGTACCAGCGTGAACCACCATG | ACGTTGGATGCATCCCCTTAGGATACTGGG |
| 7536699 | ACGTTGGATGGCCTCACAAATAGCCAAACC | ACGTTGGATGTTGCATAGCCACCTTAGACC |
| 3215497 | ACGTTGGATGTCATCCTTTTTGCGAGTCCC | ACGTTGGATGTCAATCTCCCAAGGTTCTGC |
| 1000213 | ACGTTGGATGGTGATTCACAGAGAACAAGAG | ACGTTGGATGGCTTTTTGTAGCCCTGAAGG |
| 1000212 | ACGTTGGATGTCAGCAGCAAAGTCCTCTTC | ACGTTGGATGAGGCTGTAATGCTATGTTGG |
| 1000211 | ACGTTGGATGGGAAGAACACAACTGTTGGC | ACGTTGGATGCCTGCCTTAAACACACTCAG |
| 1045017 | ACGTTGGATGACATCAGGGATGTGACTTGG | ACGTTGGATGTCCACGCTGCTCTGCAGCCATTTTAG |
| 12059520 | ACGTTGGATGCCACCACGCCCAGCTAATTT | ACGTTGGATGAAAGCGGGCAGATGGCTTGA |
| 2143118 | ACGTTGGATGCGTGCCCAGCCTAAAAATTC | ACGTTGGATGTTCTGCTGGGCATCAGTTAC |
| 12126804 | ACGTTGGATGAATTCTGTGTCTGGTGAGGG | ACGTTGGATGGCTCCTTGTTCTCCACCATG |
| 533276 | ACGTTGGATGGCCTCTGGAACTGTGAGAAG | ACGTTGGATGGTGTCTCAGTCATTTCAGGC |
| 12402174 | ACGTTGGATGAATCGCTTAGTGTCTCAGTC | ACGTTGGATGATGTTTGTTGCCTAAGCCAG |
| 2864110 | ACGTTGGATGATTCCAGCCTGGGTGACAGA | ACGTTGGATGTGACTGAGACACTAAGCGAT |
| 11581873 | ACGTTGGATGGCAGGAGAATCACATGAACC | ACGTTGGATGGCAGTAGCTTGATCACTCTC |
| 12062449 | ACGTTGGATGCGCCTGGCCTAATATTTTAT | ACGTTGGATGGTGAGAGATTACTTGAGTCC |
| 12033190 | ACGTTGGATGAGCCCGGGTGAAAGAGCAAG | ACGTTGGATGATTCATCTGCCTTGACCTC |
| 10157566 | ACGTTGGATGGCTAACACGGTGAAACTCCG | ACGTTGGATGTCCCGAGTAGTTGGGACTAC |
| 486746 | ACGTTGGATGGAGTAGTTGGGACTACAGGC | ACGTTGGATGTAACACGGTGAAACTCCGTC |
| 11802707 | ACGTTGGATGAGCCTGACCATCATGGTGAA | ACGTTGGATGAATTCTCCTGCCTCTTCCTC |
| 6668550 | ACGTTGGATGGCCTAGGTGACAGAAGACTC | ACGTTGGATGAGCCACAGCACCCAGACTTT |
| 7410946 | ACGTTGGATGTGGTGGTGGGCACCTGTGGT | ACGTTGGATGAAGCTCCATCTCCCGGGTTC |
| 7410970 | ACGTTGGATGAGTGATTCTTCTGCTTCAGC | ACGTTGGATGGACAGAGTGAGACTCTGTCT |
| 6657558 | ACGTTGGATGCTTGCAGTGAGCTGAGATCG | ACGTTGGATGGAGCTACCGAGTCTATGTTC |
| 2143119 | ACGTTGGATGATTTCTAGGACTACCCTGGG | ACGTTGGATGAGAGTGAGACCCTATCTCCA |
| 7514394 | ACGTTGGATGAGTGTGAGCACACAGGTTAG | ACGTTGGATGCCCCATTCTCTCCATATCTC |
| 11591202 | ACGTTGGATGTAATTCGCATGATCCTGTGG | ACGTTGGATGTGAGACCATCCCAAACTTTC |
| 6673991 | ACGTTGGATGTCAGGAGATCCTTCCACTTC | ACGTTGGATGTAGGGAGACCCCATCTTTAC |
| HT014-AC | ACGTTGGATGGCAATTAAAGAACTCAGCAGG | ACGTTGGATGATCTTTTGACCTCGTGATCC |
| 12034848 | ACGTTGGATGGTCCTACGTCAGTTCCGCTC | ACGTTGGATGGCATGCGCGTGGGGAGAAG |
| 2502984 | ACGTTGGATGTGGGAACCACCTTTCATCTC | ACGTTGGATGTCCACGACCATTGGTGCTAATGGCTG |
| 3831910 | ACGTTGGATGAGGGGAAATATTAAACCCGG | ACGTTGGATGCTCAAAGGAGCTCCTAATTC |
| 11379444 | ACGTTGGATGAGGGGAAATATTAAACCCGG | ACGTTGGATGCAAAGGAGCTCCTAATTCTC |
| 725119 | ACGTTGGATGAATTCTCCTGCCTCACCCTC | ACGTTGGATGTGGCCAACCTGGTAAAACCC |
| 10917425 | ACGTTGGATGGGTCTTGAACTCCTGACCTC | ACGTTGGATGCGCAGTGGCTTACACCTATA |
| 12077788 | ACGTTGGATGAGGTTCATGCCATTCTCCTG | ACGTTGGATGTAACACGGTGAAACCCCGTC |
| 12077830 | ACGTTGGATGATTACAGGTGTGAGCCACTG | ACGTTGGATGGAAGAGGCCTCGATATTAGC |
| 2473380 | ACGTTGGATGCTGTAGTCCCAGCTACTCAG | ACGTTGGATGTCTCAGCTCACTGCAACCTC |
| 2473381 | ACGTTGGATGAGCTCCAGCTTGTAAAAGCC | ACGTTGGATGTTGCCTGTCCAAAGGAGAAG |
| 11545132 | ACGTTGGATGGTAACCTGATGGACCCTATG | ACGTTGGATGACGAGGTGACCATCTGCAAT |
| 1046924 | ACGTTGGATGCCAGCCCTTAGGAAATAAAG | ACGTTGGATGATTACGAAGCATCTGCCAAC |
| 4237 | ACGTTGGATGGCTGTGGAAATTGGGTCTTG | ACGTTGGATGTCCACGCACATGGCCACATTAACTGG |
| 4375317 | ACGTTGGATGTGAGTAGCTGGGACTACAGG | ACGTTGGATGCACGGTGAAACCGTCTCTAC |
| 11584220 | ACGTTGGATGAACTTTACCACCTTCCCATC | ACGTTGGATGGAATAGGGCATGAAAGCCAG |
| 2232975 | ACGTTGGATGAAGTAGCCTGGCCGACAGTA | ACGTTGGATGCCAACATGGCAGCTTCTATC |
| 2502985 | ACGTTGGATGAAGTCCCCAAGACTGGACAG | ACGTTGGATGTCGGGAGGGTCCCGGAGAG |
| 3835423 | ACGTTGGATGAAAGCGTAAGAACTCCACCC | ACGTTGGATGAGGGCTCGTTTGCAGTCAGT |
| 2232976 | ACGTTGGATGATTCTTTGTGCCCAGGACAG | ACGTTGGATGAAGAGCAAAACCAGGCACAG |
| 2232977 | ACGTTGGATGTGTCCTGTGCCTGGTTTTGC | ACGTTGGATGTACCACACATACACCACACG |
| 2232978 | ACGTTGGATGTGTCTGGCCGTGGGTGAAAG | ACGTTGGATGAACACCATGTCTGTGCCCCT |
| 2232979 | ACGTTGGATGACGTTGGATGAACTTCTGCCC | ACGTTGGATGGAGTATCCTGAGGCCAGCAG |
| | ACCCCTCC | |
| 2232980 | ACGTTGGATGGTGGGCAGAAGTTGGAGGAC | ACGTTGGATGGGAAAAGGATGGGAAATGGG |
| 2232981 | ACGTTGGATGAGAAGTTGGAGGACCTAGGG | ACGTTGGATGTTACCCTGTGTCTCCAAGTC |
| 2232982 | ACGTTGGATGATGTACTTGACGTGAGGGAG | ACGTTGGATGTTCTCTTCCTACCACATCGG |
| 2232983 | ACGTTGGATGAGGCTTTTCACAGGTGAGGG | ACGTTGGATGTGGAGGGAGGAGAGAGAGG |
| 2232984 | ACGTTGGATGGTGGCACCATCTTGGATCTG | ACGTTGGATGGTGGGAAAAGGACAGGTCAG |
| 2232985 | ACGTTGGATGAGAGGCCTGTGGGAAAAGGA | ACGTTGGATGGTGGCACCATCTTGGATCTG |
| 2232986 | ACGTTGGATGAGGTGGCTAAGGCCAGGAAG | ACGTTGGATGCCTTCCTGACCTGTCCTTTT |
| 2745 | ACGTTGGATGCACCCAAAAACCACATGGAG | ACGTTGGATGTCCACGACTTTGTCCCCACTCTAGAG |
| 2744 | ACGTTGGATGTAATTTTATCGGAATCCAGG | ACGTTGGATGTCCACGCTGTCTCCATGTGGTTTTTG |
| 11546127 | ACGTTGGATGAGGACCCCCAAGAGTGAGTGA | ACGTTGGATGGGAAAGCTCTTTCAGAGCAA |
| 1049988 | ACGTTGGATGATAAATGAGTGCCTGGGAGG | ACGTTGGATGTCCAAGAGTTCATCAGGACC |
| 1803613 | ACGTTGGATGTCCACTGACCAGGAGGCC | ACGTTGGATGTGGACCCTGTGGAAGATA |
| 1803612 | ACGTTGGATGCCAAGGCTCCACTGACCA | ACGTTGGATGTGTGGAAGATAAGAGTTAG |
| HT014-AD | ACGTTGGATGAGCCTCTGGCAGGAACTCAG | ACGTTGGATGTCCACGTCATGCCTGGTGGGCTAAGC |
| 3209973 | ACGTTGGATGAGCACACACCTCGCCGTGTT | ACGTTGGATGTTGCGTGCCAAAGCCTGAAG |
| 3180545 | ACGTTGGATGACATCCTGTCCAGCCCTAAG | ACGTTGGATGTGATGTGGCAGCCTGTTACG |
| 3180383 | ACGTTGGATGTGTCCAGCCCTAAGGCTGCT | ACGTTGGATGGTGATGTGGCAGCCTGTTAC |
| 3177814 | ACGTTGGATGGTGATGTGGCAGCCTGTTAC | ACGTTGGATGTGTCCAGCCCTAAGGCTGCT |
| 3180546 | ACGTTGGATGGCTAAGCTCCAACTACCTCC | ACGTTGGATGTAACAGGCTGCCACATCACC |
| 3180547 | ACGTTGGATGGCTAAGCTCCAACTACCTCC | ACGTTGGATGTAACAGGCTGCCACATCACC |
| 11805398 | ACGTTGGATGGGGATCTGCAAGACAGGAGG | ACGTTGGATGACTTCTGCTTCTTAAAGAC |
| 760941 | ACGTTGGATGAGAAGGCCTCTGGGAAGAAG | ACGTTGGATGTCCACGAAAGCTGCTCTGTTGCTGAG |
| 3177813 | ACGTTGGATGAGTGCTGCAGATGGTCCAGG | ACGTTGGATGAGGTGGGTGAGGTGGGTGG |
| 3180548 | ACGTTGGATGACGTTGGATGAGGTGGGTGAG | ACGTTGGATGATTCAGTGCTGCAGATGGTC |
| | GTGGGTGG | |
| 3179864 | ACGTTGGATGGGCCTACCTTCTTCCCAGAG | ACGTTGGATGGCTATTCAGTGCTGCAGATG |
| 3177812 | ACGTTGGATGACGGGCACAGGCTATTCAGT | ACGTTGGATGTTCTTCCCAGAGGCCTTCTC |
| 3179863 | ACGTTGGATGGCTCCATAAACCCTGTTCTC | ACGTTGGATGCCATCTGCAGCACTGAATAG |
| 6692104 | ACGTTGGATGTATGACACAGAGGATGGCAC | ACGTTGGATGTGTTCCCCCTGACTCTCCTT |
| 3180549 | ACGTTGGATGCCTGAATGTCTTTGGCAATG | ACGTTGGATGTTCCTGGCTCCCACTCCTAG |
| 3179862 | ACGTTGGATGTGGTCCTAGGCTCACCTGTG | ACGTTGGATGGGAGGCCCTGAATGTCTTTG |
| 3177811 | ACGTTGGATGTCTCCACCTCCAGGTGGCGAT | ACGTTGGATGCTCACCTGTGCCATCCTCTG |
| 3177810 | ACGTTGGATGCCATCCTCTGTGTCATAGTC | ACGTTGGATGAACTGACCTGATCTCCACCT |
| 3180382 | ACGTTGGATGATCTGGGACGAACTGACCTG | ACGTTGGATGTTGCCAAAGACATTCAGGGC |
| 11546126 | ACGTTGGATGAAGAACCTGGTGTTCAGCAG | ACGTTGGATGTAAGGGTTGGTACAACCACC |
| 7551384 | ACGTTGGATGATGTAGGCCACATCATCACG | ACGTTGGATGAAAACCCTGGGTTCCACCTC |
| 12141511 | ACGTTGGATGAGTTTACACCTAGCGAAGGC | ACGTTGGATGAAAATGGCAGCACGATTGGG |
| 2473382 | ACGTTGGATGCAGTTTACACCTAGCGAAGG | ACGTTGGATGAAAATGGCAGCACGATTGGG |
| 12038166 | ACGTTGGATGTCAGCCTCTGCCCATGCCA | ACGTTGGATGAGTAGCCTAAGTTGGGCGGG |
| 6666908 | ACGTTGGATGCGGGCCCTTTAAGTGCTGAC | ACGTTGGATGAACCCGAGTGACATGTTTCC |
| 12041159 | ACGTTGGATGTTGCAAACTGGACTTCCAGC | ACGTTGGATGGGTGTGTGCCAAACCATGAA |
| 12031592 | ACGTTGGATGTTCATCACGCAGGATGTCAC | ACGTTGGATGCGACGGCAGGTTAGAAAATG |
| 1062598 | ACGTTGGATGTGTGGAAAGTGGAGAGCTAC | ACGTTGGATGAAATCACATTCAGCGTGGAG |
| 11714 | ACGTTGGATGAGCGTGGAGATTTTCCAGAG | AGCGGATAACAATTTCACACAGGCAGAGGCCATTTGT |
| | | GGAAAG |
| 3203593 | ACGTTGGATGTCATGGAGGTTCTTCCCAAG | ACGTTGGATGTTCCAGAGATGCTGGAGTTG |
| 1042436 | ACGTTGGATGAGAAGTCAGAGAGCAAGGG | ACGTTGGATGTGGATGGAATCCGCGAGCTG |
| 2076343 | ACGTTGGATGTCATGGTATGCCCTCAACAC | ACGTTGGATGGCTGTCAAGGAAAAAGCCTG |
| 12131539 | ACGTTGGATGAGACAGACAGGAGCTCACAG | ACGTTGGATGAGCATGGACAGACAACTGTG |
| 11583935 | ACGTTGGATGGGCCCACAGAGGTAAAAAAC | ACGTTGGATGTTTCTGTCTGGCCCTTTCAG |
| 12140263 | ACGTTGGATGACACGCATGGGCATAAGAAG | ACGTTGGATGTTCCATGTTCCTGGTGTCTC |
| 3835225 | ACGTTGGATGTCAGAAAAGGTCTCCCTGAG | ACGTTGGATGTCTGCCAGCAGTATCTTTCC |
| 2473374 | ACGTTGGATGTCCCTCATGCTGCCTGAGAT | ACGTTGGATGTCCTGAAGCAGGTGGGAATG |
| 2256179 | ACGTTGGATGTGTCTCGAGCTTCACAGATC | ACGTTGGATGTCCACGAATCCAGGTGAGAGACATGG |
| 974698 | ACGTTGGATGAGGAGCCTCTCAAACGGAAC | ACGTTGGATGTTTCAGACTGAGCTGGGAAG |
| 1018396 | ACGTTGGATGAGGTGAGTCTGAGGCAAAAC | ACGTTGGATGTCCACGCTGCCATGAAACAGAGAAGC |
| 719399 | ACGTTGGATGTAACTCCCTTGACTGTCTCC | ACGTTGGATGAATGGTGGACACAGTCATGG |
| 719400 | ACGTTGGATGTAGGTGTCATGGCAGTGGAC | ACGTTGGATGTCCACGCACCCCAGGGATCATGAAAG |
| 2502986 | ACGTTGGATGGGATTGCTTGAGTCTGAGAG | ACGTTGGATGTCACTCTGTCATTCGGCTAG |
| 6679378 | ACGTTGGATGAGATGTGCAAATGGGCTTCC | ACGTTGGATGTTGTGATCTGCCCATCTCAG |
| 2473375 | ACGTTGGATGGCAACTTCACTTTTCTGAGC | ACGTTGGATGTAGCCTCAGTTGCCACAATC |
| 2473376 | ACGTTGGATGGGTACGGCTACAGAAAAGAG | ACGTTGGATGGTGACTAATCAAGTGCAGAC |
| 12136455 | ACGTTGGATGAATCACTTGAACCCAGGAGG | ACGTTGGATGTTTGAGACAGAGTCTCACTC |
| 2502987 | ACGTTGGATGAAGTGATTCTCCTGCCTCAG | ACGTTGGATGTAGTGGATAGAAACCCCGTC |
| 11551767 | ACGTTGGATGGGAAGTAGTCATCTTTGGAG | ACGTTGGATGCTGAAAACTCTCCTCTATGG |
| 11591205 | ACGTTGGATGACATGGTGAAACGCTGTCTC | ACGTTGGATGTGAGTAGCTGGGATTACAGG |
| 12083298 | ACGTTGGATGTCTCCTGACCTCATGATCTG | ACGTTGGATGAATCCCATACGACTGGTGAC |
| 12138604 | ACGTTGGATGGGTTCAAGTGATTCTCCCAC | ACGTTGGATGCAAAAAATTAGCCGGTCGTG |
| 12136042 | ACGTTGGATGAAAAAATTAGCCGGTCGTGG | ACGTTGGATGACCTCCCAGGTTCAAGTGAT |
| 12138606 | ACGTTGGATGCACCTCCCAGGTTCAAGTGA | ACGTTGGATGAAAATTAGCCGGTCGTGGTG |
| 12136045 | ACGTTGGATGAGGCTGAGGTGGGAGAATCA | ACGTTGGATGGTACAGTGGCATGATCTCGG |
| 12116596 | ACGTTGGATGTGAGGTGGGAGAATCACTTG | ACGTTGGATGTGGAGTACAGTGGCATGATC |
| 12138610 | ACGTTGGATGAATCACTTGAACCTGGGAGG | ACGTTGGATGTTAGACAGAGTCTTGCTCTG |
| 12136069 | ACGTTGGATGAGACAGAGTCTTGCTCTGTC | ACGTTGGATGAATCACTTGAACCTGGGAGG |
| 12116617 | ACGTTGGATGATCATGCCACTGTACTCCAG | ACGTTGGATGCTGTGTCTCTGTGTCTTCAC |
| 7540056 | ACGTTGGATGTGGCTAACACGATGAAACCC | ACGTTGGATGCCGGGTTCAAGTGATTCTCC |
| 12044452 | ACGTTGGATGCTATCTTTGTGAAGCAGGAG | ACGTTGGATGTCTAAAACCCTCTATGCCTG |
| 2076344 | ACGTTGGATGGCCCTAGCCCTCAATGAAAG | ACGTTGGATGCTTCTACTTGCTTCAAAGGC |
| 11588187 | ACGTTGGATGTTCTCCAGTTTCTCTCCCAC | ACGTTGGATGCAATACAGTGAGACTCCATC |
| 6696932 | ACGTTGGATGTTCCGGGTTCAAGCGATTCT | ACGTTGGATGTGGTGAAACCCCATCTCTAC |
| 12025531 | ACGTTGGATGAGTGAGTGCTTGTTGGAAAC | ACGTTGGATGATAGCAGTAAGCAGAAGCAG |
| 6682888 | ACGTTGGATGAGTGAGTGCTTGTTGGAAAC | ACGTTGGATGGCAGTAAGCAGAAGCAGAAC |
| 2982390 | ACGTTGGATGCCGTGCCTGGCCTTTTTTTA | ACGTTGGATGAAGATCGTGCCACTGTACTC |
| 2179394 | ACGTTGGATGTAGCAACCTGTTGGGTGGAG | ACGTTGGATGTGAGTTGCCTCCTCTGACCT |
| 2473377 | ACGTTGGATGTCTGACCTCCTGGAATCATC | ACGTTGGATGAAGGCAGGGAGCTTAGCAAC |
| 2179395 | ACGTTGGATGTTTGCAAAGGGTGGTCAGAG | ACGTTGGATGGGGTTCCTCATCTGGCTTTT |
| 2502978 | ACGTTGGATGACACTGTGTGATCCGCTAAG | ACGTTGGATGGTACATGTTCCAGCTGCTTC |
| 2502979 | ACGTTGGATGACTGTTATTTATCGAGCCCC | ACGTTGGATGTTCATGAGACAGGGACTTTG |
| 6698535 | ACGTTGGATGATTTGCCAGGCACATACAAG | ACGTTGGATGAGACAGAATGAGACTGTATC |
| 2502980 | ACGTTGGATGACCTGGTGAAATCCTGTCTC | ACGTTGGATGCCAGGTTCAAGTGATTCTCC |
| 10554242 | ACGTTGGATGAGTAATCTTCCCGAGGTGAC | ACGTTGGATGTTGTGCTCTAGAGCCACACT |
| 6697805 | ACGTTGGATGATCAAAACGGAGAGAGGCTG | ACGTTGGATGTGACCGATGGCTCGTCTGGTG |
| 6424114 | ACGTTGGATGTGGCTAACATGGCGAAACCC | ACGTTGGATGCACCACGCCCGGCTTTTTTT |
| 5773058 | ACGTTGGATGATGCTACACCGTCTGGAAAG | ACGTTGGATGTTATCTAGGTGTACTTTCTG |
| 4649114 | ACGTTGGATGGCCCTGAGGCAAAGAAAATG | ACGTTGGATGAGTGAGACTCCATCTCAAAC |
| 6424115 | ACGTTGGATGACCCCACTAATTGGTCACAG | ACGTTGGATGCAACACAGTGAGACTTCGTC |

TABLE 39

| dbSNP rs# | Extend Primer | Term Mix |
|---|---|---|
| 627451 | GGATTACAGGCATGAGCCAC | ACG |
| 12082331 | CCGTTCTCCTGCCTCAGC | ACT |
| 2267960 | GACCTTATAATCAGCATTGGAT | ACT |
| 7519640 | GGCCAGGGTCTGGCGCG | ACT |
| 12086741 | CGGCCTCCTCGCCGGAA | ACG |
| 3934189 | AAGAACGCGTTTAACTTAAT | ACT |
| 12135415 | GTCTCCCGGGTTCAGGCA | ACT |
| 12088267 | CCAGGCTGGTCTTGAACTCA | ACT |
| 12084665 | CAGGCTGGTCTTGAACTCAG | ACG |
| 550850 | AACAGAGTTACAGCAATGTCTA | ACT |
| 557227 | GGAGATGGAGTTTTGCT | CGT |
| 12137502 | GGACAACAAGAGCAAAACTCC | CGT |
| 12118858 | GACAACAAGAGCAAAACTCCAT | ACG |
| 2294495 | GAATGTTCTGGAAACCA | ACG |
| 12401650 | AGCGCCCTCGGGATGCC | ACG |
| 2235541 | AAGAAACCTGGAAAGCCA | ACG |
| 520713 | CTGTCCTTCTCTTTCTTTA | ACG |
| 550252 | GACGGTAATTGGCCTGTATC | ACT |
| HT014-AA | CTGTAATCCCAGCACTTTGG | ACT |
| 12093683 | CACTTTAGGAGGCCAAGGCA | ACT |
| 10917421 | CCCAGCTACTTGGGAGGAT | ACG |
| 2744873 | TTGGTCTTCATCTTAGTAGT | CGT |
| 12091764 | ATTCTCACGCCTCAGCCTC | ACG |
| HT014-AB | TTGTGTACAAGCACTTTTCCAT | CGT |
| 2076345 | CCATGCTTCCGCCAGTAG | ACG |
| 2076346 | GAGTTAAAAACTAGTGCTGA | ACT |
| 2473379 | TCCCTTGAATCCAGGAGTTC | ACT |
| 6679300 | CCAGGCTGGAGTGCAGTG | ACT |
| 11811395 | GCCCTCTTACTGTTTTTTTATAG | CGT |
| 8179467 | CTAATTGGGTCTTCCAATTTATTT | CGT |
| 8179468 | GGGTCTTCCAATTTATTTATTT | CGT |
| 10917423 | GAACCACCATGCCTGGCC | ACT |
| 7536699 | CAAACCAGTAAACAGCA | ACT |
| 3215497 | CCCACCTTTTTGTTTTTTTTTTTT | ACG |
| 1000213 | CAAGAGATTAAAGTGCTGG | CGT |
| 1000212 | AAGTCCTCTTCCCAAGCCAA | ACG |
| 1000211 | TTGGGAAGAGGACTTTGCTG | ACG |
| 1045017 | TGTGACTTGGTGCGAAGA | ACT |
| 12059520 | CGTTGGCCAGGCTGGTCT | ACT |
| 2143118 | CTCAAGTTCAACAGTCATTT | ACG |
| 12126804 | GGCCCTCTTCTTGGTTTGCA | ACT |
| 533276 | TGCCTAAGCCAGTCAGT | ACG |
| 12402174 | TAGTGTCTCAGTCATTTCAGG | ACT |
| 2864110 | TGACAGAGTGAGACTTC | CGT |
| 11581873 | CACATGAACCCGGGAGGCA | ACT |
| 12062449 | ATCTCACTATGTTGCCCAGG | ACG |
| 12033190 | GTGAAAGAGCAAGACCCTGT | ACG |
| 10157566 | AGCCGAGCGTGGTGGCG | ACT |
| 486746 | CTCGCCACCACGCTCGG | ACT |
| 11802707 | GGTGTGAGCCTGTAATTCCA | ACT |
| 6668550 | GTGACAGAAGACTCCATCTC | CGT |
| 7410946 | CGGGAGGCTGAGGCAGGA | ACT |
| 7410970 | CGCACACCACCATACCCAG | ACT |
| 6657558 | CTGCACTCCAGCCTGGG | ACT |
| 2143119 | ACTACCCTGGGGCAGGTAA | ACT |
| 7514394 | CTGCATGCGTGGACACAAG | ACT |
| 11591202 | GCATGATCCTGTGGATTAGC | ACT |
| 6673991 | GAGCCACCATGCTGGGCTA | ACT |
| HT014-AC | GAACTCAGCAGGCCGGGC | ACT |
| 12034848 | TCCGCTCGACCCCACCC | ACG |
| 2502984 | CTTTCATCTCCCTAGGCTC | ACT |
| 3831910 | ACCCGGTAAGAAGAAAA | CGT |
| 11379444 | TATTAAACCCGGTAAGAAGAAAA | CGT |
| 725119 | CCAGGCCCAGCTAATTTTTGT | CGT |
| 10917425 | CTCCTGACCTCAGGTAATCC | ACT |
| 12077788 | CCTGCCACCACGCCCGG | ACT |
| 12077830 | TGAGCCACTGCGCCCGG | ACT |
| 2473380 | GCATGAGAGTCACTTGAACC | ACG |
| 2473381 | GTAAAAGCCAGGCAAAG | ACT |
| 11545132 | TCTGCTGGGTTGGCAGATG | CGT |
| 1046924 | GGGTAACCTGATGGACCCT | ACT |
| 4237 | GGCATCTGGCAGTCATGG | ACT |
| 4375317 | GCTCCCGCCACCGCACC | ACG |
| 11584220 | ACCTCTTAAGGCCTCGGGT | ACT |
| 2232975 | GCCGACAGTACGGAACC | CGT |
| 2502985 | TCACTCCCCACCCCCAT | ACG |
| 3835423 | GGTGCTCCACGCCCCCC | ACG |
| 2232976 | GTGCTGTGTGGGGCTGA | ACT |
| 2232977 | CTGGTTTTGCTCTTGAC | ACG |
| 2232978 | TGAAAGGGGACCCTTAC | ACG |
| 2232979 | AGGTAGCAGCCAAACAG | ACT |
| 2232980 | AGTTGGAGGACCTAGGG | ACT |
| 2232981 | AGGGGAAGCCTTCCTGT | ACG |
| 2232982 | GTGCCTGTAGGAGGGAC | ACT |
| 2232983 | GGTGAGTGAGCTGTGCC | CGT |
| 2232984 | GGTCGAGCCCCTGTCCC | ACG |
| 2232985 | AAAGGACAGGTCAGGAA | ACT |
| 2232986 | GGCCAGGAAGGAAGGCCC | ACT |
| 2745 | CACATGGAGACAGAAGAC | ACT |
| 2744 | TATCGGAATCCAGGACACAA | ACT |
| 11546127 | GGCTCTGGCACAAAACCTC | CGT |
| 1049988 | GAGGAGGAGGTTTTGTGCCA | ACT |
| 1803613 | ACCAGGAGGCCGAGGTCT | ACG |
| 1803612 | TCCACTGACCAGGAGGCC | ACT |
| HT014-AD | TGGAGCTGCTGGGGCCAA | ACT |
| 3209973 | CCTCGCCGTGTTGCAGGT | ACT |
| 3180545 | CTAAGGCTGCTGTCCACCC | CGT |
| 3180383 | CCAGCTCCTCTTGGGCCA | ACT |
| 3177814 | CAGCCTGTTACGCCAACCC | CGT |
| 3180546 | CCCGTACAAGGTGGTGGC | CGT |
| 3180547 | CAGATCCCGTACAAGGTGGT | ACT |
| 11805398 | CAGGAGGTAGTTGGAGCTTA | ACT |
| 760941 | AAGAAGGTAGGCCCCCCC | ACT |
| 3177813 | CTATGGAGAAGGCCTCTGG | ACT |
| 3180548 | CCTACCTTCTTCCCAGA | ACT |
| 3179864 | CAGAGGCCTTCTCCATA | ACT |
| 3177812 | ACAGGCTATTCAGTGCTGCA | ACT |
| 3179863 | GTGGGCCAGATCTACAA | CGT |
| 6692104 | ACAGGTGAGCCTAGGAC | ACT |
| 3180549 | ACTATGACACAGAGGATG | ACT |
| 3179862 | CCTGTGCCATCCTCTGTGT | CGT |
| 3177811 | GGAGGCCCTGAATGTCTTTG | ACT |
| 3177810 | CCAAAGACATTCAGGGCCTC | CGT |
| 3180382 | CTGATCTCCACCTCCAG | ACT |
| 11546126 | GTGTACGGGAACCCCCAG | ACG |
| 7551384 | CCAGACACACATTCCCC | ACT |
| 12141511 | AGACAGAACCCCTTCCTGC | ACT |
| 2473382 | CTGAGCAGACAGAACCC | ACG |
| 12038166 | CCGGTCACTCCTGGCTCA | ACT |
| 6666908 | GTGCTGACGGCCCACTC | CGT |
| 12041159 | CCTTAGTCCCGTGGGATCG | ACT |
| 12031592 | CTAGCTCTACTACTTTTCATTG | ACT |
| 1062598 | ATCTGCCAACTCCAGCATCT | ACG |
| 11714 | GATGCTGGAGTTGGCAGA | ACG |
| 3203593 | GGCCATTTGTGGAAAGTGGA | ACT |
| 1042436 | GCAGGGTGGAGCCGTGTA | ACT |
| 2076343 | CAGGGAAAACATCAGCATGG | ACT |
| 12131539 | CACAGGCAGCTGGTGCCC | ACG |
| 11583935 | GACAATTGTGAGGCAGAGAG | ACT |
| 12140263 | CAGGGTGACTTTGACAGTTCA | ACT |
| 3835225 | GGTGGCTTTCAGACTGGAAC | ACG |
| 2473374 | CTGCCTGAGATGGACCC | ACG |
| 2256179 | CTTCACAGATCAGAACAC | ACT |
| 974698 | CAGATCATCTCCCTCCC | ACG |
| 1018396 | GGCAAAACTTCATCCTGATCA | ACG |
| 719399 | GTCTCCCCAACACAGAG | ACG |
| 719400 | ACTTCCTGCATGACAGCAGA | ACG |
| 2502986 | AGTAAGCCGAGATCACA | CGT |
| 6679378 | CGGGCACGGTGGCTCAC | ACT |
| 2473375 | GATGACCCTTTGGTCATA | ACG |
| 2473376 | CGGGACCAAATCATGCA | ACT |
| 12136455 | GTGAGCCAAGATCACGCCA | ACT |
| 2502987 | GCCTCAGCCTCCCAAGTAG | CGT |
| 11551767 | GAGCTGCCTCAGAGCTCTT | ACG |
| 11591205 | AAATTAGCCGGGCGTGGTG | ACT |
| 12083298 | CGGCCTCCCAAAGTGCTG | CGT |
| 12138604 | TCTCCCACCTCAGCCTCC | ACG |
| 12136042 | CTACTCGGGAGGCTGAGG | ACG |
| 12138606 | CCAGGTTCAAGTGATTCTCC | ACG |
| 12136045 | GTGGGAGAATCACTTGAACC | ACG |
| 12116596 | TTGAACCTGGGAGGTGGAG | ACT |
| 12138610 | GTGGAGCTTGCAGTGAGCC | ACT |
| 12136069 | TGTCGCCCAGGCTGGAGT | ACT |
| 12116617 | CACTGTACTCCAGCCTGGG | ACG |
| 7540056 | GTGGCAGGTACCTGTAATCC | ACG |
| 12044452 | TTTGTGAAGCAGGAGTTGTCA | ACT |
| 2076344 | CAATGAAAGGCCTTTCTC | ACG |
| 11588187 | CTCTAACTTAATCTTTCCACAAAA | CGT |
| 6696932 | CCTCCAGAGTAGATGGGATT | CGT |
| 12025531 | GTGTAAGTTCCCGAAGGGCA | ACT |
| 6682888 | AACAATAGTGTGTAAGTTCC | ACG |
| 2982390 | GGAAATGGAGTCTTCCT | ACT |
| 2179394 | GTAGGGGCGAGTGATGA | CGT |
| 2473377 | TCATCACTCGCCCCTAC | ACG |
| 2179395 | GAAAGATGAGAGAGAGATGT | ACT |
| 2502978 | CGCTAAGGATCTCAACC | ACG |
| 2502979 | GCCCCTATTATGTCCCAGG | ACT |
| 6698535 | AAGAGCCATTTACATATTTATTTAT | CGT |
| 2502980 | AGCCAGGTGTGGTGGTGC | ACT |
| 10554242 | AGGTGACATGGGGAGTTACT | ACT |
| 6697805 | CCAGCTTCGGAAGTCAC | ACT |
| 6424114 | GCGAAACCCCCGTCTCT | CGT |
| 5773058 | CCTGGATAGATTTTTTTTTTTTT | ACG |
| 4649114 | TTCTCTCTTTTGTTTTGTTG | ACT |
| 6424115 | GGGTACAAACTTTATTTAAAACAA | ACT |

### Genetic Analysis of Allelotyping Results

Allelotyping results are shown for cases and controls in Table 40. The allele frequency for the A2 allele is noted in the fifth and sixth columns for breast cancer pools and control pools, respectively, where “AF” is allele frequency. SNPs with blank allele frequencies were untyped.

TABLE 40

| | Position in | | | | | | | |
|---|---|---|---|---|---|---|---|---|
| | SEQ ID | Chromosome | A1/A2 | | | | | Breast Cancer |
| dbSNP rs# | NO: 5 | Position | Allele | Case AF | Control AF | p-Value | OR | Associated Allele |
| 627451 | 215 | 23536215 | G/A | G = 0.753 | G = 0.765 | 0.681 | 1.06 | A |
| | | | | A = 0.247 | A = 0.235 | | | |
| 12082331 | 385 | 23536385 | G/C | G = 0.999 | G = 1.000 | 0.913 | 2.58 | C |
| | | | | C = 0.001 | C = 0.000 | | | |
| 2267960 | 2019 | 23538019 | T/C | T = 0.734 | T = 0.742 | 0.796 | 1.04 | C |
| | | | | C = 0.266 | C = 0.258 | | | |
| 7519640 | 3112 | 23539112 | C/G | C = 0.77 | C = 0.778 | 0.82 | 1.04 | G |
| | | | | G = 0.230 | G = 0.222 | | | |
| 12086741 | 3437 | 23539437 | G/A | G = 0.999 | G = 0.999 | 0.979 | 1.15 | A |
| | | | | A = 0.001 | A = 0.001 | | | |
| 3934189 | 4326 | 23540326 | T/C | T = 1.000 | T = 1.000 | 0.948 | 0.43 | T |
| | | | | C = 0.000 | C = 0.000 | | | |
| 12135415 | 4523 | 23540523 | A/G | A = | A = 0.997 | | | |
| | | | | G = | G = 0.003 | | | |
| 12088267 | 4645 | 23540645 | A/G | A = 0.001 | A = 0 | 0.963 | 0.00 | A |
| | | | | G = 0.999 | G = 1.000 | | | |
| 12084665 | 4646 | 23540646 | C/T | C = 0.875 | C = | | | |
| | | | | T = 0.125 | T = | | | |
| 550850 | 5830 | 23541830 | T/C | T = 0.906 | T = 0.917 | 0.624 | 1.15 | C |
| | | | | C = 0.094 | C = 0.083 | | | |
| 557227 | 6556 | 23542556 | G/T | G = 0.999 | G = 0.998 | 0.947 | 0.51 | G |
| | | | | T = 0.001 | T = 0.002 | | | |
| 12137502 | 6568 | 23542568 | T/A | T = 0.001 | T = 0.001 | 0.989 | 1.33 | A |
| | | | | A = 0.999 | A = 0.999 | | | |
| 12118858 | 6570 | 23542570 | C/T | C = 1.000 | C = 1.000 | 0.993 | 1.10 | T |
| | | | | T = 0.000 | T = 0.000 | | | |
| 2294495 | 9351 | 23545351 | C/T | C = 0.781 | C = 0.723 | 0.0342 | 0.73 | C |
| | | | | T = 0.219 | T = 0.277 | | | |
| 12401650 | 10874 | 23546874 | C/T | C = 0.999 | C = 0.999 | 0.991 | 1.08 | T |
| | | | | T = 0.001 | T = 0.001 | | | |
| 2235541 | 10926 | 23546926 | C/T | C = 0.929 | C = 0.936 | 0.712 | 1.11 | T |
| | | | | T = 0.071 | T = 0.064 | | | |
| 520713 | 11462 | 23547462 | G/A | G = 0.997 | G = 0.996 | 0.96 | 0.77 | G |
| | | | | A = 0.003 | A = 0.004 | | | |
| 550252 | 12415 | 23548415 | T/C | T = 0.002 | T = 0.001 | 0.969 | 0.75 | T |
| | | | | C = 0.998 | C = 0.999 | | | |
| HT014-AA | 12668 | 23548668 | A/G | A = 0.116 | A = 0.076 | 0.0822 | 0.62 | A |
| | | | | G = 0.884 | G = 0.924 | | | |
| 12093683 | 13886 | 23549886 | G/C | G = | G = 0.991 | | | |
| | | | | C = | C = 0.009 | | | |
| 10917421 | 15005 | 23551005 | G/A | G = 1.000 | G = 0.999 | 0.89 | 0.12 | G |
| | | | | A = 0.000 | A = 0.001 | | | |
| 2744873 | 15387 | 23551387 | A/T | A = 0.002 | A = 0.001 | 0.899 | 0.12 | A |
| | | | | T = 0.998 | T = 0.999 | | | |
| 12091764 | 15639 | 23551639 | G/A | G = | G = 0.95 | | | |
| | | | | A = | A = 0.050 | | | |
| HT014-AB | 15761 | 23551761 | A/T | A = 0.344 | A = 0.425 | 0.0165 | 1.41 | T |
| | | | | T = 0.656 | T = 0.575 | | | |
| 2076345 | 16969 | 23552969 | C/T | C = 0.672 | C = 0.616 | 0.0738 | 0.78 | C |
| | | | | T = 0.328 | T = 0.384 | | | |
| 2076346 | 17124 | 23553124 | T/C | T = 0.799 | T = 0.822 | 0.425 | 1.16 | C |
| | | | | C = 0.201 | C = 0.178 | | | |
| 2473379 | 17369 | 23553369 | A/G | A = 0.994 | A = 0.994 | 0.982 | 0.95 | A |
| | | | | G = 0.006 | G = 0.006 | | | |
| 6679300 | 17523 | 23553523 | C/G | C = 0.003 | C = 0.005 | 0.854 | 1.93 | G |
| | | | | G = 0.997 | G = 0.995 | | | |
| 11811395 | 18158 | 23554158 | T/A | T = 1.000 | T = 1.000 | 0.994 | 1.07 | A |
| | | | | A = 0.000 | A = 0.000 | | | |
| 8179467 | 18220 | 23554220 | T/A | T = 0.831 | T = 0.734 | 0.00376 | 0.56 | T |
| | | | | A = 0.169 | A = 0.266 | | | |
| 8179468 | 18224 | 23554224 | T/A | T = | T = 0.099 | | | |
| | | | | A = | A = 0.901 | | | |
| 10917423 | 18538 | 23554538 | A/G | A = 0.793 | A = 0.831 | 0.142 | 1.28 | G |
| | | | | G = 0.207 | G = 0.169 | | | |
| 7536699 | 18810 | 23554810 | G/C | G = 1.000 | G = 0.998 | 0.884 | 0.45 | G |
| | | | | C = 0.000 | C = 0.002 | | | |
| 3215497 | 20282 | 23556282 | —/T | — = 0.549 | — = 0.528 | 0.514 | 0.92 | — |
| | | | | T = 0.451 | T = 0.472 | | | |
| 1000213 | 20495 | 23556495 | C/A | C = 0 | C = 0.001 | 0.908 | | A |
| | | | | A = 1.000 | A = 0.999 | | | |
| 1000212 | 20633 | 23556633 | C/T | C = 1.000 | C = 0.999 | 0.843 | 0.14 | C |
| | | | | T = 0.000 | T = 0.001 | | | |
| 1000211 | 20659 | 23556659 | G/A | G = 0.999 | G = 0.998 | 0.896 | 0.54 | G |
| | | | | A = 0.001 | A = 0.002 | | | |
| 1045017 | 21603 | 23557603 | A/G | A = 0.368 | A = 0.342 | 0.445 | 0.90 | A |
| | | | | G = 0.632 | G = 0.658 | | | |
| 12059520 | 24075 | 23560075 | C/G | C = 0.995 | C = 0.992 | 0.81 | 0.60 | C |
| | | | | G = 0.005 | G = 0.008 | | | |
| 2143118 | 24191 | 23560191 | C/T | C = 0.999 | C = 0.999 | 0.922 | 1.73 | T |
| | | | | T = 0.001 | T = 0.001 | | | |
| 12126804 | 25010 | 23561010 | T/C | T = 0.002 | T = 0.005 | 0.7 | 3.45 | C |
| | | | | C = 0.998 | C = 0.995 | | | |
| 533276 | 25124 | 23561124 | C/T | C = 0.999 | C = 0.999 | 0.971 | 0.83 | C |
| | | | | T = 0.001 | T = 0.001 | | | |
| 12402174 | 25145 | 23561145 | G/C | G = 1.000 | G = 1.000 | 0.942 | 0.00 | G |
| | | | | C = 0 | C = 0.000 | | | |
| 2864110 | 25222 | 23561222 | C/A | C = 0.001 | C = 0.001 | 0.995 | 1.05 | A |
| | | | | A = 0.999 | A = 0.999 | | | |
| 11581873 | 25288 | 23561288 | T/C | T = 0.001 | T = 0.001 | 0.991 | 0.40 | T |
| | | | | C = 0.999 | C = 0.999 | | | |
| 12062449 | 26080 | 23562080 | G/A | G = 0.999 | G = 0.999 | 0.951 | 0.73 | G |
| | | | | A = 0.001 | A = 0.001 | | | |
| 12033190 | 26556 | 23562556 | C/T | C = 0.998 | C = 0.999 | 0.925 | 1.38 | T |
| | | | | T = 0.002 | T = 0.001 | | | |
| 10157566 | 27416 | 23563416 | T/C | T = | T = 0.006 | | | |
| | | | | C = | C = 0.994 | | | |
| 486746 | 27432 | 23563432 | C/G | C = 1.000 | C = 0.998 | 0.84 | 0.36 | C |
| | | | | G = 0.000 | G = 0.002 | | | |
| 11802707 | 29841 | 23565841 | A/G | A = 0.004 | A = 0.004 | 0.976 | 1.22 | G |
| | | | | G = 0.996 | G = 0.996 | | | |
| 6668550 | 29955 | 23565955 | C/A | C = 0.001 | C = 0.002 | 0.919 | 2.07 | A |
| | | | | A = 0.999 | A = 0.998 | | | |
| 7410946 | 30160 | 23566160 | A/G | A = 0.015 | A = 0.02 | 0.773 | 1.32 | G |
| | | | | G = 0.985 | G = 0.980 | | | |
| 7410970 | 30294 | 23566294 | G/C | G = 0.993 | G = 0.997 | 0.814 | 2.03 | C |
| | | | | C = 0.007 | C = 0.003 | | | |
| 6657558 | 31039 | 23567039 | C/G | C = 0.019 | C = 0.038 | 0.227 | 2.14 | G |
| | | | | G = 0.981 | G = 0.962 | | | |
| 2143119 | 32071 | 23568071 | T/C | T = 0.001 | T = 0.002 | 0.887 | 9.57 | C |
| | | | | C = 0.999 | C = 0.998 | | | |
| 7514394 | 33698 | 23569698 | T/C | T = 0.624 | T = 0.586 | 0.23 | 0.85 | T |
| | | | | C = 0.376 | C = 0.414 | | | |
| 11591202 | 35555 | 23571555 | A/C | A = 0.744 | A = 0.67 | 0.0142 | 0.70 | A |
| | | | | C = 0.256 | C = 0.330 | | | |
| 6673991 | 36658 | 23572658 | A/G | A = 1.000 | A = 0.999 | 0.912 | 0.04 | A |
| | | | | G = 0.000 | G = 0.001 | | | |
| HT014-AC | 37720 | 23573720 | G/C | G = 0.069 | G = 0.0630 | 0.736 | 0.90 | G |
| | | | | C = 0.931 | C = 0.937 | | | |
| 12034848 | 38304 | 23574304 | C/T | C = 0.623 | C = 0.799 | 5.36E−08 | 2.40 | T |
| | | | | T = 0.377 | T = 0.201 | | | |
| 2502984 | 39008 | 23575008 | T/C | T = 0.197 | T = 0.16 | 0.145 | 0.77 | T |
| | | | | C = 0.803 | C = 0.840 | | | |
| 3831910 | 39985 | 23575985 | A/— | A = 0.152 | A = 0.01 | 2.26E−08 | 0.05 | A |
| | | | | — = 0.848 | — = 0.990 | | | |
| 11379444 | 39989 | 23575989 | —/T | — = 0.144 | — = 0.146 | 0.957 | 1.01 | T |
| | | | | T = 0.856 | T = 0.854 | | | |
| 725119 | 40683 | 23576683 | T/A | T = 0 | T = 0.002 | 0.892 | | A |
| | | | | A = 1.000 | A = 0.998 | | | |
| 10917425 | 42158 | 23578158 | T/C | T = 0.865 | T = 0.856 | 0.691 | 0.93 | T |
| | | | | C = 0.135 | C = 0.144 | | | |
| 12077788 | 44369 | 23580369 | C/G | C = 1.000 | C = 1.000 | 0.986 | 0.79 | C |
| | | | | G = 0.000 | G = 0.000 | | | |
| 12077830 | 44503 | 23580503 | C/G | C = 0.999 | C = 1.000 | 0.95 | 1.83 | G |
| | | | | G = 0.001 | G = 0.000 | | | |
| 2473380 | 45382 | 23581382 | C/T | C = 0.708 | C = 0.709 | 0.97 | 1.01 | T |
| | | | | T = 0.292 | T = 0.291 | | | |
| 2473381 | 46171 | 23582171 | A/C | A = 1.000 | A = 1.000 | 0.943 | 0.32 | A |
| | | | | C = 0.000 | C = 0.000 | | | |
| 11545132 | 47276 | 23583276 | G/T | G = 0.999 | G = 0.999 | 0.99 | 1.06 | T |
| | | | | T = 0.001 | T = 0.001 | | | |
| 1046924 | 47299 | 23583299 | T/C | T = 1.000 | T = 1.000 | 0.976 | 0.00 | T |
| | | | | C = 0 | C = 0.000 | | | |
| 4237 | 47604 | 23583604 | A/G | A = 0.664 | A = 0.612 | 0.0897 | 0.80 | A |
| | | | | G = 0.336 | G = 0.388 | | | |
| 4375317 | 48462 | 23584462 | G/A | G = 0.808 | G = 0.8 | 0.799 | 0.95 | G |
| | | | | A = 0.192 | A = 0.200 | | | |
| 11584220 | 50786 | 23586786 | A/C | A = 0.001 | A = 0 | 0.93 | 0.00 | A |
| | | | | C = 0.999 | C = 1.000 | | | |
| 2232975 | 51057 | 23587057 | G/T | G = 1.000 | G = 1.000 | 0.929 | 0.32 | G |
| | | | | T = 0.000 | T = 0.000 | | | |
| 2502985 | 51310 | 23587310 | G/A | G = 0 | G = 0.001 | 0.958 | | A |
| | | | | A = 1.000 | A = 0.999 | | | |
| 3835423 | 51996 | 23587996 | C/— | C = 0.112 | C = 0.1 | 0.555 | 0.88 | C |
| | | | | — = 0.888 | — = 0.900 | | | |
| 2232976 | 52546 | 23588546 | T/G | T = 0.998 | T = 0.998 | 0.984 | 1.08 | G |
| | | | | G = 0.002 | G = 0.002 | | | |
| 2232977 | 52602 | 23588602 | C/T | C = 1.000 | C = 0.999 | 0.897 | 0.27 | C |
| | | | | T = 0.000 | T = 0.001 | | | |
| 2232978 | 52725 | 23588725 | G/A | G = 1.000 | G = 0.999 | 0.875 | 0.09 | G |
| | | | | A = 0.000 | A = 0.001 | | | |
| 2232979 | 52858 | 23588858 | A/C | A = 0.002 | A = 0.002 | 0.982 | 0.86 | A |
| | | | | C = 0.998 | C = 0.998 | | | |
| 2232980 | 52935 | 23588935 | T/G | T = 0.001 | T = 0.001 | 0.977 | 1.70 | G |
| | | | | G = 0.999 | G = 0.999 | | | |
| 2232981 | 52948 | 23588948 | C/T | C = 0.999 | C = 1.000 | 0.973 | 1.28 | T |
| | | | | T = 0.001 | T = 0.000 | | | |
| 2232982 | 53102 | 23589102 | T/C | T = 0.176 | T = 0.075 | 0.000007 | 0.38 | T |
| | | | | C = 0.824 | C = 0.925 | | | |
| 2232983 | 54160 | 23590160 | C/A | C = 0.999 | C = 0.999 | 0.937 | 1.48 | A |
| | | | | A = 0.001 | A = 0.001 | | | |
| 2232984 | 54794 | 23590794 | C/T | C = 0.999 | C = 0.999 | 0.977 | 0.86 | C |
| | | | | T = 0.001 | T = 0.001 | | | |
| 2232985 | 54798 | 23590798 | T/C | T = 0.001 | T = 0.005 | 0.756 | 4.62 | C |
| | | | | C = 0.999 | C = 0.995 | | | |
| 2232986 | 54853 | 23590853 | T/C | T = 0.789 | T = 0.831 | 0.152 | 1.31 | C |
| | | | | C = 0.211 | C = 0.169 | | | |
| 2745 | 55422 | 23591422 | A/G | A = 0.815 | A = 0.833 | 0.472 | 1.13 | G |
| | | | | G = 0.185 | G = 0.167 | | | |
| 2744 | 55460 | 23591460 | C/G | C = 0.964 | C = 0.95 | 0.415 | 0.70 | C |
| | | | | G = 0.036 | G = 0.050 | | | |
| 11546127 | 55645 | 23591645 | G/T | G = 0.999 | G = 0.999 | 0.996 | 1.03 | T |
| | | | | T = 0.001 | T = 0.001 | | | |
| 1049988 | 55660 | 23591660 | T/C | T = 0.003 | T = 0.005 | 0.872 | 1.45 | C |
| | | | | C = 0.997 | C = 0.995 | | | |
| 1803613 | 55729 | 23591729 | C/T | C = 1.000 | C = 1.000 | 0.983 | 1.25 | T |
| | | | | T = 0.000 | T = 0.000 | | | |
| 1803612 | 55736 | 23591736 | T/G | T = 0.026 | T = 0.039 | 0.539 | 1.51 | G |
| | | | | G = 0.974 | G = 0.961 | | | |
| HT014-AD | 55821 | 23591821 | T/C | T = 0.003 | T = 0.005 | 0.877 | 1.44 | C |
| | | | | C = 0.997 | C = 0.995 | | | |
| 3209973 | 55970 | 23591970 | T/G | T = 0.001 | T = 0.001 | 0.961 | 2.36 | G |
| | | | | G = 0.999 | G = 0.999 | | | |
| 3180545 | 56150 | 23592150 | C/A | C = 0.999 | C = 0.999 | 0.967 | 0.78 | C |
| | | | | A = 0.001 | A = 0.001 | | | |
| 3180383 | 56167 | 23592167 | A/G | A = 0.004 | A = 0.005 | 0.932 | 1.31 | G |
| | | | | G = 0.996 | G = 0.995 | | | |
| 3177814 | 56171 | 23592171 | G/T | G = 0.999 | G = 1.000 | 0.932 | 1.86 | T |
| | | | | T = 0.001 | T = 0.000 | | | |
| 3180546 | 56210 | 23592210 | G/T | G = 0.002 | G = 0.001 | 0.957 | 0.61 | G |
| | | | | T = 0.998 | T = 0.999 | | | |
| 3180547 | 56213 | 23592213 | A/C | A = 0.001 | A = 0.002 | 0.984 | 1.22 | C |
| | | | | C = 0.999 | C = 0.998 | | | |
| 11805398 | 56259 | 23592259 | T/G | T = 0.001 | T = 0.001 | 0.997 | 0.95 | T |
| | | | | G = 0.999 | G = 0.999 | | | |
| 760941 | 56461 | 23592461 | T/C | T = 0.672 | T = 0.626 | 0.148 | 0.82 | T |
| | | | | C = 0.328 | C = 0.374 | | | |
| 3177813 | 56480 | 23592480 | T/C | T = 0.002 | T = 0.001 | 0.947 | 0.50 | T |
| | | | | C = 0.998 | C = 0.999 | | | |
| 3180548 | 56486 | 23592486 | A/G | A = 0.001 | A = 0.002 | 0.899 | 8.26 | G |
| | | | | G = 0.999 | G = 0.998 | | | |
| 3179864 | 56499 | 23592499 | A/G | A = 0.002 | A = 0.001 | 0.848 | 0.16 | A |
| | | | | G = 0.998 | G = 0.999 | | | |
| 3177812 | 56510 | 23592510 | T/C | T = 0.003 | T = 0.002 | 0.925 | 0.57 | T |
| | | | | C = 0.997 | C = 0.998 | | | |
| 3179863 | 56543 | 23592543 | G/T | G = 1.000 | G = 1.000 | 0.994 | 0.64 | G |
| | | | | T = 0.000 | T = 0.000 | | | |
| 6692104 | 56827 | 23592827 | G/C | G = 1.000 | G = 1.000 | 0.985 | 0.68 | G |
| | | | | C = 0.000 | C = 0.000 | | | |
| 3180549 | 56846 | 23592846 | G/C | G = 0.001 | G = 0.001 | 0.909 | 0.13 | G |
| | | | | C = 0.999 | C = 0.999 | | | |
| 3179862 | 56859 | 23592859 | C/A | C = 0.999 | C = 0.997 | 0.806 | 0.37 | C |
| | | | | A = 0.001 | A = 0.003 | | | |
| 3177811 | 56870 | 23592870 | G/C | G = 0.002 | G = 0.002 | 0.996 | 0.95 | G |
| | | | | C = 0.998 | C = 0.998 | | | |
| 3177810 | 56890 | 23592890 | C/A | C = 1.000 | C = 1.000 | 1 | | |
| | | | | A = 0 | A = 0 | | | |
| 3180382 | 56907 | 23592907 | A/C | A = 0.002 | A = 0.001 | 0.877 | 0.25 | A |
| | | | | C = 0.998 | C = 0.999 | | | |
| 11546126 | 57767 | 23593767 | C/T | C = 0.001 | C = 0.002 | 0.903 | 2.73 | T |
| | | | | T = 0.999 | T = 0.998 | | | |
| 7551384 | 58774 | 23594774 | C/G | C = 0.002 | C = 0.001 | 0.978 | 0.80 | C |
| | | | | G = 0.998 | G = 0.999 | | | |
| 12141511 | 59444 | 23595444 | T/C | T = 0.001 | T = 0 | 0.991 | 0.00 | T |
| | | | | C = 0.999 | C = 1.000 | | | |
| 2473382 | 59452 | 23595452 | G/A | G = 0.999 | G = 0.999 | 0.917 | 0.55 | G |
| | | | | A = 0.001 | A = 0.001 | | | |
| 12038166 | 59753 | 23595753 | A/G | A = 0.001 | A = 0.002 | 0.907 | 6.61 | G |
| | | | | G = 0.999 | G = 0.998 | | | |
| 6666908 | 60766 | 23596766 | C/A | C = 1.000 | C = 0.999 | 0.908 | 0.50 | C |
| | | | | A = 0.000 | A = 0.001 | | | |
| 12041159 | 61135 | 23597135 | G/C | G = 0.37 | G = 0.264 | 0.000762 | 0.61 | G |
| | | | | C = 0.630 | C = 0.736 | | | |
| 12031592 | 61339 | 23597339 | A/C | A = 0.483 | A = 0.543 | 0.15 | 1.27 | C |
| | | | | C = 0.517 | C = 0.457 | | | |
| 1062598 | 61922 | 23597922 | C/T | C = 0.999 | C = 1.000 | 0.874 | 2.87 | T |
| | | | | T = 0.001 | T = 0.000 | | | |
| 11714 | 61942 | 23597942 | C/T | C = 0.152 | C = 0.143 | 0.721 | 0.93 | C |
| | | | | T = 0.848 | T = 0.857 | | | |
| 3203593 | 61958 | 23597958 | A/G | A = 0.002 | A = 0.001 | 0.912 | 0.30 | A |
| | | | | G = 0.998 | G = 0.999 | | | |
| 1042436 | 62068 | 23598068 | T/C | T = 0.001 | T = 0.002 | 0.981 | 1.21 | C |
| | | | | C = 0.999 | C = 0.998 | | | |
| 2076343 | 62601 | 23598601 | A/G | A = 0.809 | A = 0.737 | 0.00747 | 0.66 | A |
| | | | | G = 0.191 | G = 0.263 | | | |
| 12131539 | 62661 | 23598661 | G/A | G = 1.000 | G = 1.000 | 0.919 | 0.06 | G |
| | | | | A = 0.000 | A = 0.000 | | | |
| 11583935 | 62882 | 23598882 | T/G | T = 0 | T = 0.001 | 0.934 | | G |
| | | | | G = 1.000 | G = 0.999 | | | |
| 12140263 | 63949 | 23599949 | A/C | A = 1.000 | A = | | | |
| | | | | C = 0 | C = | | | |
| 3835225 | 64732 | 23600732 | —/AG | — = 1.000 | — = 1.000 | 0.997 | 0.92 | — |
| | | | | AG = 0.000 | AG = 0.000 | | | |
| 2473374 | 65145 | 23601145 | C/T | C = 0.001 | C = 0.002 | 0.953 | 1.72 | T |
| | | | | T = 0.999 | T = 0.998 | | | |
| 2256179 | 65780 | 23601780 | A/G | A = 0.658 | A = 0.602 | 0.0693 | 0.79 | A |
| | | | | G = 0.342 | G = 0.398 | | | |
| 974698 | 66260 | 23602260 | C/T | C = 0.437 | C = 0.499 | 0.0501 | 1.28 | T |
| | | | | T = 0.563 | T = 0.501 | | | |
| 1018396 | 67087 | 23603087 | C/T | C = 0.447 | C = 0.522 | 0.0188 | 1.35 | T |
| | | | | T = 0.553 | T = 0.478 | | | |
| 719399 | 67937 | 23603937 | G/A | G = 0.206 | G = 0.206 | 0.99 | 1.00 | A |
| | | | | A = 0.794 | A = 0.794 | | | |
| 719400 | 68196 | 23604196 | G/A | G = 0.901 | G = 0.931 | 0.112 | 1.49 | A |
| | | | | A = 0.099 | A = 0.069 | | | |
| 2502986 | 68767 | 23604767 | G/T | G = 0.453 | G = 0.518 | 0.0505 | 1.29 | T |
| | | | | T = 0.547 | T = 0.482 | | | |
| 6679378 | 69482 | 23605482 | T/C | T = 0.741 | T = 0.696 | 0.132 | 0.80 | T |
| | | | | C = 0.259 | C = 0.304 | | | |
| 2473375 | 70020 | 23606020 | C/T | C = 0.469 | C = 0.522 | 0.117 | 1.24 | T |
| | | | | T = 0.531 | T = 0.478 | | | |
| 2473376 | 72759 | 23608759 | A/G | A = 0.002 | A = 0.002 | 0.991 | 0.95 | A |
| | | | | G = 0.998 | G = 0.998 | | | |
| 12136455 | 72911 | 23608911 | G/C | G = 0.999 | G = 0.997 | 0.882 | 0.59 | G |
| | | | | C = 0.001 | C = 0.003 | | | |
| 2502987 | 73331 | 23609331 | C/A | C = 0.946 | C = 0.944 | 0.934 | 0.96 | C |
| | | | | A = 0.054 | A = 0.056 | | | |
| 11551767 | 74250 | 23610250 | C/T | C = 1.000 | C = 0.999 | 0.922 | 0.46 | C |
| | | | | T = 0.000 | T = 0.001 | | | |
| 11591205 | 74486 | 23610486 | T/C | T = 0.133 | T = 0.139 | 0.817 | 1.05 | C |
| | | | | C = 0.867 | C = 0.861 | | | |
| 12083298 | 74921 | 23610921 | A/T | A = 0.001 | A = 0.001 | 0.99 | 0.52 | A |
| | | | | T = 0.999 | T = 0.999 | | | |
| 12138604 | 75065 | 23611065 | G/A | G = 0.682 | G = 0.688 | 0.825 | 1.03 | A |
| | | | | A = 0.318 | A = 0.312 | | | |
| 12136042 | 75077 | 23611077 | C/T | C = 0.981 | C = 0.978 | 0.873 | 0.88 | C |
| | | | | T = 0.019 | T = 0.022 | | | |
| 12138606 | 75078 | 23611078 | G/A | G = 0.002 | G = 0.003 | 0.969 | 1.38 | A |
| | | | | A = 0.998 | A = 0.997 | | | |
| 12136045 | 75096 | 23611096 | C/T | C = 0.705 | C = 0.698 | 0.795 | 0.96 | C |
| | | | | T = 0.295 | T = 0.302 | | | |
| 12116596 | 75108 | 23611108 | C/G | C = 0.002 | C = 0.001 | 0.994 | 0.94 | C |
| | | | | G = 0.998 | G = 0.999 | | | |
| 12138610 | 75121 | 23611121 | A/G | A = 0.586 | A = 0.578 | 0.787 | 0.97 | A |
| | | | | G = 0.414 | G = 0.422 | | | |
| 12136069 | 75136 | 23611136 | T/C | T = 0.286 | T = 0.294 | 0.789 | 1.04 | C |
| | | | | C = 0.714 | C = 0.706 | | | |
| 12116617 | 75150 | 23611150 | C/T | C = | C = 0.697 | | | |
| | | | | T = | T = 0.303 | | | |
| 7540056 | 75594 | 23611594 | G/A | G = | G = 0.996 | | | |
| | | | | A = | A = 0.004 | | | |
| 12044452 | 77120 | 23613120 | T/G | T = 0.001 | T = 0.055 | 0.00645 | 57.90 | G |
| | | | | G = 0.999 | G = 0.945 | | | |
| 2076344 | 77407 | 23613407 | G/A | G = 0.893 | G = 0.901 | 0.707 | 1.09 | A |
| | | | | A = 0.107 | A = 0.099 | | | |
| 11588187 | 78522 | 23614522 | T/A | T = 0.509 | T = 0.57 | 0.0684 | 1.28 | A |
| | | | | A = 0.491 | A = 0.430 | | | |
| 6696932 | 78656 | 23614656 | T/A | T = 0 | T = 0 | 1 | | T |
| | | | | A = 1.000 | A = 1.000 | | | |
| 12025531 | 78902 | 23614902 | T/C | T = 0.004 | T = 0.003 | 0.905 | 0.67 | T |
| | | | | C = 0.996 | C = 0.997 | | | |
| 6682888 | 78911 | 23614911 | G/A | G = 1.000 | G = 1.000 | 0.935 | 0.19 | G |
| | | | | A = 0.000 | A = 0.000 | | | |
| 2982390 | 81305 | 23617305 | G/C | G = 0.487 | G = 0.57 | 0.0151 | 1.40 | C |
| | | | | C = 0.513 | C = 0.430 | | | |
| 2179394 | 81717 | 23617717 | A/T | A = 0.999 | A = 0.997 | 0.859 | 0.57 | A |
| | | | | T = 0.001 | T = 0.003 | | | |
| 2473377 | 81735 | 23617735 | C/T | C = 0.58 | C = 0.546 | 0.279 | 0.87 | C |
| | | | | T = 0.420 | T = 0.454 | | | |
| 2179395 | 81840 | 23617840 | T/C | T = 0.564 | T = 0.63 | 0.0527 | 1.32 | C |
| | | | | C = 0.436 | C = 0.370 | | | |
| 2502978 | 82511 | 23618511 | C/T | C = 1.000 | C = 1.000 | 0.98 | 0.47 | C |
| | | | | T = 0.000 | T = 0.000 | | | |
| 2502979 | 82790 | 23618790 | A/C | A = 0.686 | A = 0.629 | 0.0805 | 0.78 | A |
| | | | | C = 0.314 | C = 0.371 | | | |
| 6698535 | 83323 | 23619323 | T/A | T = 1.000 | T = 1.000 | 1 | | |
| | | | | A = 0 | A = 0 | | | |
| 2502980 | 83464 | 23619464 | T/C | T = 0.948 | T = 1.000 | 0.0305 | | C |
| | | | | C = 0.052 | C = 0 | | | |
| 6697805 | 85471 | 23621471 | A/C | A = 0.687 | A = 0.774 | 0.00312 | 1.56 | C |
| | | | | C = 0.313 | C = 0.226 | | | |
| 6424114 | 85688 | 23621688 | C/A | C = 0.002 | C = 0.002 | 0.943 | 0.69 | C |
| | | | | A = 0.998 | A = 0.998 | | | |
| 5773058 | 86196 | 23622196 | —/TT | — = 0.547 | — = 0.491 | 0.0906 | 0.80 | — |
| | | | | TT = 0.453 | TT = 0.509 | | | |
| 4649114 | 86323 | 23622323 | T/G | T = 0.003 | T = 0.004 | 0.977 | 1.08 | G |
| | | | | G = 0.997 | G = 0.996 | | | |
| 6424115 | 86706 | 23622706 | A/G | A = 0.632 | A = 0.591 | 0.213 | 0.84 | A |
| | | | | G = 0.368 | G = 0.409 | | | |
| 10554242 | 83916-83920 | 23619916\hat{ }23619919 | CACT/— | CACT = 0.476 | CACT = 0.541 | 0.0458 | 1.30 | — |
| | | | | — = 0.524 | — = 0.459 | | | |

FIG. 1E shows proximal SNPs in and around the HT014/LOC148902/LYPLA2/GALE region for females. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1E can be determined by consulting Table 40. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Wed at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 16

### DPF3 Proximal SNPs

It has been discovered that a polymorphic variation (rs1990440) in the DPF3 gene is associated with the occurrence of breast cancer (see Examples 1 and 2). Subsequently, SNPs proximal to the incident SNP (rs 1990440) were identified and allelotyped in breast cancer sample sets and control sample sets as described in Examples 1 and 2. Approximately 627 allelic variants located within the DPF3 gene were identified and allelotyped. The polymorphic variants are set forth in Table 41. The chromosome position provided in column four of Table 41 is based on Genome “Build 34” of NCBI's GenBank.

TABLE 41

| | Position in SEQ | | Chromosome | | Genome | Deduced |
|---|---|---|---|---|---|---|
| dbSNP rs# | ID NO: 6 | Chromosome | Position | Allele Variants | Letter | Iupac |
| 11846274 | 207 | 14 | 71066357 | g/a | a | R |
| 2526935 | 486 | 14 | 71066636 | t/g | a | M |
| 929328 | 1745 | 14 | 71067895 | t/c | a | R |
| 3742840 | 1922 | 14 | 71068072 | a/g | t | Y |
| 9671739 | 2190 | 14 | 71068340 | a/c | c | M |
| 8006208 | 2590 | 14 | 71068740 | c/t | c | Y |
| 8006230 | 2637 | 14 | 71068787 | t/c | c | Y |
| 8007836 | 2804 | 14 | 71068954 | c/t | t | Y |
| 8006889 | 2806 | 14 | 71068956 | g/a | g | R |
| 8010823 | 2895 | 14 | 71069045 | c/t | c | Y |
| 8011029 | 3109 | 14 | 71069259 | a/t | a | W |
| 10649920 | 3185 | 14 | 71069335 | —/aa | a | N |
| 2803956 | 3355 | 14 | 71069505 | a/g | a | R |
| 2803957 | 3642 | 14 | 71069792 | c/t | t | Y |
| 2526934 | 3805 | 14 | 71069955 | t/c | a | R |
| 9671558 | 4236 | 14 | 71070386 | a/g | g | R |
| 2526933 | 4331 | 14 | 71070481 | c/g | c | S |
| 2803959 | 4509 | 14 | 71070659 | t/c | t | Y |
| 2526932 | 4959 | 14 | 71071109 | c/t | a | R |
| 2803960 | 5009 | 14 | 71071159 | g/a | a | R |
| 12434675 | 5676 | 14 | 71071826 | g/a | g | R |
| 4899435 | 6507 | 14 | 71072657 | g/c | c | S |
| 2526931 | 6695 | 14 | 71072845 | c/t | a | R |
| 2803961 | 6717 | 14 | 71072867 | t/c | t | Y |
| 2803962 | 7131 | 14 | 71073281 | c/t | c | Y |
| 12590634 | 7873 | 14 | 71074023 | c/t | c | Y |
| 11327712 | 7922 | 14 | 71074072 | —/c | c | N |
| 2803963 | 8652 | 14 | 71074802 | a/g | g | R |
| 11844834 | 9817 | 14 | 71075967 | g/a | g | R |
| 11320415 | 10272 | 14 | 71076422 | —/a | a | N |
| 8012752 | 10823 | 14 | 71076973 | t/c | c | Y |
| 11623878 | 11465 | 14 | 71077615 | t/c | c | Y |
| 11623926 | 11639 | 14 | 71077789 | c/t | c | Y |
| 12434442 | 12177 | 14 | 71078327 | t/c | c | Y |
| 2526930 | 12604 | 14 | 71078754 | g/a | c | Y |
| 12435306 | 13363 | 14 | 71079513 | g/a | a | R |
| 10144028 | 13454 | 14 | 71079604 | c/g | c | S |
| 4899436 | 13540 | 14 | 71079690 | c/t | c | Y |
| 7140593 | 13923 | 14 | 71080073 | a/c | a | M |
| 2107687 | 14507 | 14 | 71080657 | t/c | a | R |
| 991882 | 16550 | 14 | 71082700 | c/a | t | K |
| 3327 | 17641 | 14 | 71083791 | c/t | t | Y |
| 2526928 | 18903 | 14 | 71085053 | t/c | a | R |
| 7401336 | 19395 | 14 | 71085545 | a/g | a | R |
| 2526926 | 19527 | 14 | 71085677 | t/a | a | W |
| 994760 | 20369 | 14 | 71086519 | t/c | a | R |
| 994759 | 20505 | 14 | 71086655 | g/t | a | M |
| 994758 | 20562 | 14 | 71086712 | c/t | g | R |
| 11848404 | 20907 | 14 | 71087057 | a/t | t | W |
| 2526925 | 20949 | 14 | 71087099 | c/t | a | R |
| 11844236 | 21278 | 14 | 71087428 | t/g | g | K |
| 2526924 | 21314 | 14 | 71087464 | c/t | a | R |
| 2803965 | 21905 | 14 | 71088055 | c/g | c | S |
| 2526923 | 22252 | 14 | 71088402 | g/c | g | S |
| 7152467 | 22941 | 14 | 71089091 | c/t | c | Y |
| 11623462 | 23542 | 14 | 71089692 | c/g | g | S |
| 2803966 | 24677 | 14 | 71090827 | c/t | c | Y |
| 2526922 | 25009 | 14 | 71091159 | t/c | a | R |
| 2526920 | 25618 | 14 | 71091768 | c/t | a | R |
| 11848135 | 26082 | 14 | 71092232 | t/c | t | Y |
| 11629210 | 26136 | 14 | 71092286 | c/a | c | M |
| 2803967 | 26491 | 14 | 71092641 | c/t | t | Y |
| 16455 | 28873 | 14 | 71095023 | —/gcc | g | N |
| 757655 | 28886 | 14 | 71095036 | g/a | c | Y |
| 5809581 | 29105 | 14 | 71095255 | —/tctgtaat | t | N |
| 3057139 | 29106 | 14 | 71095256 | —/ctgtaatt | c | N |
| 2247658 | 29572 | 14 | 71095722 | c/t | t | Y |
| 11625609 | 30640 | 14 | 71096790 | c/t | c | Y |
| 8005580 | 30801 | 14 | 71096951 | a/g | a | R |
| 8010472 | 30962 | 14 | 71097112 | g/a | g | R |
| 2526919 | 31662 | 14 | 71097812 | t/c | a | R |
| 2803968 | 34374 | 14 | 71100524 | g/a | a | R |
| 11623943 | 34494 | 14 | 71100644 | t/c | c | Y |
| 11623946 | 34501 | 14 | 71100651 | g/c | c | S |
| 1557926 | 34544 | 14 | 71100694 | c/g | c | S |
| 12434611 | 35493 | 14 | 71101643 | a/g | g | R |
| 2526918 | 35594 | 14 | 71101744 | t/g | a | M |
| 5809582 | 35901 | 14 | 71102051 | —/t | t | N |
| 8008279 | 35940 | 14 | 71102090 | c/g | c | S |
| 2803969 | 36064 | 14 | 71102214 | t/c | t | Y |
| 2526917 | 36105 | 14 | 71102255 | t/g | a | M |
| 8008774 | 36160 | 14 | 71102310 | t/c | c | Y |
| 2803970 | 36161 | 14 | 71102311 | t/g | t | K |
| 8010255 | 36308 | 14 | 71102458 | t/c | t | Y |
| 2997642 | 36335 | 14 | 71102485 | a/g | a | R |
| 2803971 | 36365 | 14 | 71102515 | g/a | a | R |
| 11313760 | 36488 | 14 | 71102638 | —/g | g | N |
| 2803972 | 36576 | 14 | 71102726 | g/t | t | K |
| 4903038 | 36641 | 14 | 71102791 | a/g | g | R |
| 2526916 | 36933 | 14 | 71103083 | a/t | t | W |
| 4903040 | 36955 | 14 | 71103105 | c/t | c | Y |
| 2803974 | 36967 | 14 | 71103117 | t/a | t | W |
| 2803975 | 37243 | 14 | 71103393 | c/t | t | Y |
| 2803976 | 38329 | 14 | 71104479 | c/t | t | Y |
| 2803977 | 38367 | 14 | 71104517 | t/g | g | K |
| 10139909 | 38476 | 14 | 71104626 | c/t | t | Y |
| 722945 | 38510 | 14 | 71104660 | g/a | c | Y |
| 917069 | 39396 | 14 | 71105546 | a/g | t | Y |
| 2803978 | 39703 | 14 | 71105853 | c/a | a | M |
| 2332890 | 39801 | 14 | 71105951 | a/g | g | R |
| 1989638 | 40078 | 14 | 71106228 | t/c | g | R |
| 10679174 | 40260 | 14 | 71106410 | ggg/— | g | N |
| 2526915 | 40513 | 14 | 71106663 | c/t | a | R |
| 2526914 | 40889 | 14 | 71107039 | t/c | g | R |
| 11158964 | 41091 | 14 | 71107241 | g/a | g | R |
| 11158965 | 41872 | 14 | 71108022 | a/g | a | R |
| 740081 | 42112 | 14 | 71108262 | a/g | c | Y |
| 740080 | 42292 | 14 | 71108442 | t/g | a | M |
| 10140582 | 42373 | 14 | 71108523 | t/c | c | Y |
| 2526913 | 44877 | 14 | 71111027 | a/g | c | Y |
| 4899437 | 45124 | 14 | 71111274 | c/g | c | S |
| 10145678 | 45831 | 14 | 71111981 | t/c | t | Y |
| 2107686 | 46030 | 14 | 71112180 | t/c | g | R |
| 2107685 | 46270 | 14 | 71112420 | t/c | a | R |
| 2107684 | 46309 | 14 | 71112459 | g/a | c | Y |
| 7154188 | 46545 | 14 | 71112695 | c/g | g | S |
| 4903041 | 47162 | 14 | 71113312 | c/t | c | Y |
| 7159637 | 47267 | 14 | 71113417 | t/c | c | Y |
| 6574089 | 49077 | 14 | 71115227 | a/g | a | R |
| 12589753 | 49377 | 14 | 71115527 | a/g | g | R |
| 4899438 | 50126 | 14 | 71116276 | g/c | c | S |
| 4903042 | 50385 | 14 | 71116535 | g/t | g | K |
| 4903043 | 50403 | 14 | 71116553 | a/t | t | W |
| 2803980 | 50422 | 14 | 71116572 | g/a | a | R |
| 10483848 | 50893 | 14 | 71117043 | t/a | a | W |
| 2240344 | 52031 | 14 | 71118181 | g/c | c | S |
| 2286068 | 52245 | 14 | 71118395 | a/g | t | Y |
| 11339669 | 52594 | 14 | 71118744 | —/g | g | N |
| 3854 | 52874 | 14 | 71119024 | a/g | t | Y |
| 11463 | 53011 | 14 | 71119161 | t/g | c | M |
| 1383 | 53656 | 14 | 71119806 | a/t | t | W |
| 1381 | 53662 | 14 | 71119812 | c/g | c | S |
| 1377 | 53711 | 14 | 71119861 | t/c | a | R |
| 11158966 | 53798 | 14 | 71119948 | a/g | g | R |
| 11158967 | 53840 | 14 | 71119990 | a/g | g | R |
| 11158968 | 53873 | 14 | 71120023 | c/t | c | Y |
| 11158969 | 53884 | 14 | 71120034 | a/g | g | R |
| 11158970 | 53937 | 14 | 71120087 | a/g | g | R |
| 11158971 | 54032 | 14 | 71120182 | g/a | g | R |
| 11849782 | 54352 | 14 | 71120502 | c/g | g | S |
| 10137616 | 55430 | 14 | 71121580 | c/t | t | Y |
| 757572 | 55920 | 14 | 71122070 | g/a | c | Y |
| 10140566 | 56081 | 14 | 71122231 | g/t | t | K |
| 2023480 | 56227 | 14 | 71122377 | a/t | t | W |
| 2526911 | 57350 | 14 | 71123500 | t/a | t | W |
| 4903044 | 57493 | 14 | 71123643 | c/t | c | Y |
| 11845162 | 57682 | 14 | 71123832 | g/c | g | S |
| 12147210 | 58092 | 14 | 71124242 | c/t | c | Y |
| 2997643 | 58731 | 14 | 71124881 | g/t | g | K |
| 2997644 | 58826 | 14 | 71124976 | a/t | a | W |
| 3814867 | 60916 | 14 | 71127066 | t/c | a | R |
| 2286067 | 62047 | 14 | 71128197 | a/g | t | Y |
| 1060570 | 62080 | 14 | 71128230 | t/g | c | M |
| 2332891 | 62665 | 14 | 71128815 | c/g | c | S |
| 2021736 | 62809 | 14 | 71128959 | g/a | t | Y |
| 7160830 | 63629 | 14 | 71129779 | t/c | c | Y |
| 2526909 | 65432 | 14 | 71131582 | a/g | t | Y |
| 7140963 | 65590 | 14 | 71131740 | g/c | g | S |
| 10143667 | 65876 | 14 | 71132026 | a/g | g | R |
| 2803981 | 67118 | 14 | 71133268 | t/c | c | Y |
| 768840 | 67348 | 14 | 71133498 | t/c | g | R |
| 5809587 | 68230 | 14 | 71134380 | t/— | t | N |
| 2023479 | 68262 | 14 | 71134412 | a/g | c | Y |
| 4903045 | 68912 | 14 | 71135062 | t/c | c | Y |
| 4903046 | 69799 | 14 | 71135949 | a/g | g | R |
| 5809588 | 70027 | 14 | 71136177 | —/g | g | N |
| 10220555 | 73335 | 14 | 71139485 | g/a | g | R |
| 2906132 | 74093 | 14 | 71140243 | g/a | c | Y |
| 11848482 | 74114 | 14 | 71140264 | t/g | t | K |
| 10129954 | 74592 | 14 | 71140742 | t/c | c | Y |
| 2526921 | 75061 | 14 | 71141211 | c/t | a | R |
| 4903047 | 75683 | 14 | 71141833 | g/t | g | K |
| 11846357 | 75857 | 14 | 71142007 | a/g | a | R |
| 10136385 | 76457 | 14 | 71142607 | g/c | c | S |
| 12588242 | 77130 | 14 | 71143280 | t/a | a | W |
| 11621517 | 77947 | 14 | 71144097 | c/t | t | Y |
| 2526910 | 78338 | 14 | 71144488 | c/t | g | R |
| 2526941 | 79626 | 14 | 71145776 | a/g | t | Y |
| 2803944 | 80497 | 14 | 71146647 | g/a | g | R |
| 12436770 | 80578 | 14 | 71146728 | a/g | g | R |
| 10220279 | 81171 | 14 | 71147321 | c/a | c | M |
| 10220280 | 81243 | 14 | 71147393 | g/c | c | S |
| 11844112 | 81321 | 14 | 71147471 | a/c | c | M |
| 12432455 | 82634 | 14 | 71148784 | g/c | g | S |
| 8015338 | 83269 | 14 | 71149419 | g/a | g | R |
| 3832969 | 83514 | 14 | 71149664 | cagc/— | a | N |
| 9671972 | 85367 | 14 | 71151517 | a/g | a | R |
| 9671975 | 85423 | 14 | 71151573 | c/t | c | Y |
| 7159986 | 86006 | 14 | 71152156 | t/c | c | Y |
| 11848220 | 86515 | 14 | 71152665 | t/g | t | K |
| 5016369 | 86936 | 14 | 71153086 | g/c | c | S |
| 1986423 | 87201 | 14 | 71153351 | a/g | a | R |
| 2332909 | 87205 | 14 | 71153355 | c/t | c | Y |
| 8013337 | 88313 | 14 | 71154463 | g/a | a | R |
| 11445680 | 89477 | 14 | 71155627 | —/g | a | N |
| 4903048 | 90243 | 14 | 71156393 | t/c | c | Y |
| 12434793 | 90735 | 14 | 71156885 | t/c | c | Y |
| 2332910 | 91990 | 14 | 71158140 | g/c | g | S |
| 4410017 | 92879 | 14 | 71159029 | a/g | g | R |
| 4577019 | 92880 | 14 | 71159030 | a/g | a | R |
| 2332911 | 93386 | 14 | 71159536 | a/g | g | R |
| 4996747 | 93397 | 14 | 71159547 | t/g | t | K |
| 10140820 | 93680 | 14 | 71159830 | g/t | t | K |
| 12431623 | 95585 | 14 | 71161735 | t/c | t | Y |
| 6574090 | 95818 | 14 | 71161968 | g/t | t | K |
| 8008750 | 96203 | 14 | 71162353 | c/g | g | S |
| 11627420 | 96911 | 14 | 71163061 | g/a | g | R |
| 10150185 | 98014 | 14 | 71164164 | a/g | g | R |
| 10150218 | 98229 | 14 | 71164379 | a/c | c | M |
| 9646164 | 98235 | 14 | 71164385 | t/g | g | K |
| 6574091 | 99173 | 14 | 71165323 | c/t | t | Y |
| 2332912 | 100505 | 14 | 71166655 | a/g | g | R |
| 7155359 | 101613 | 14 | 71167763 | a/g | a | R |
| 2332913 | 101660 | 14 | 71167810 | c/t | c | Y |
| 4544184 | 101681 | 14 | 71167831 | g/t | t | K |
| 4337224 | 102165 | 14 | 71168315 | g/a | g | R |
| 2332914 | 103763 | 14 | 71169913 | c/t | t | Y |
| 4280167 | 104990 | 14 | 71171140 | g/a | g | R |
| 4903049 | 105319 | 14 | 71171469 | g/t | t | K |
| 3742837 | 105447 | 14 | 71171597 | t/g | c | M |
| 10139937 | 105471 | 14 | 71171621 | a/g | a | R |
| 12434292 | 105515 | 14 | 71171665 | a/g | a | R |
| 11620671 | 105598 | 14 | 71171748 | a/t | t | W |
| 10140184 | 105674 | 14 | 71171824 | c/a | c | M |
| 2332915 | 106432 | 14 | 71172582 | c/g | g | S |
| 2332916 | 106988 | 14 | 71173138 | c/t | c | Y |
| 8012759 | 107076 | 14 | 71173226 | c/t | c | Y |
| 10149272 | 107991 | 14 | 71174141 | g/c | c | S |
| 11158973 | 108221 | 14 | 71174371 | a/g | a | R |
| 4903050 | 108410 | 14 | 71174560 | g/t | t | K |
| 10666445 | 108762 | 14 | 71174912 | ctta/— | c | N |
| 2536143 | 108924 | 14 | 71175074 | a/t | a | W |
| 10638231 | 109378 | 14 | 71175528 | ttttttttttttt/— | t | N |
| | | | | (SEQ ID NO: 4934) | | |
| 12431636 | 109442 | 14 | 71175592 | a/g | g | R |
| 8005904 | 109619 | 14 | 71175769 | c/t | t | Y |
| 11621058 | 109820 | 14 | 71175970 | c/t | c | Y |
| 10133224 | 110530 | 14 | 71176680 | t/c | c | Y |
| 12433185 | 110956 | 14 | 71177106 | a/t | a | W |
| 740974 | 111965 | 14 | 71178115 | a/g | a | R |
| 740973 | 111995 | 14 | 71178145 | g/c | c | S |
| 740972 | 112040 | 14 | 71178190 | t/c | c | Y |
| 740971 | 112058 | 14 | 71178208 | a/g | a | R |
| 4243642 | 112592 | 14 | 71178742 | c/g | g | S |
| 4415952 | 113044 | 14 | 71179194 | c/t | t | Y |
| 2215589 | 113263 | 14 | 71179413 | g/a | t | Y |
| 4343211 | 113301 | 14 | 71179451 | t/c | c | Y |
| 2536142 | 113513 | 14 | 71179663 | t/c | g | R |
| 11293340 | 113639 | 14 | 71179789 | —/a | a | N |
| 10151620 | 114092 | 14 | 71180242 | c/g | c | S |
| 2286840 | 114432 | 14 | 71180582 | t/g | a | M |
| 8023089 | 114643 | 14 | 71180793 | t/a | a | W |
| 8008009 | 114980 | 14 | 71181130 | g/a | g | R |
| 11419089 | 115312 | 14 | 71181462 | —/t | t | N |
| 10694676 | 115315 | 14 | 71181465 | —/tt | t | N |
| 2536141 | 115377 | 14 | 71181527 | c/t | g | R |
| 765247 | 115625 | 14 | 71181775 | c/a | t | K |
| 7147626 | 115740 | 14 | 71181890 | t/c | t | Y |
| 10132629 | 115747 | 14 | 71181897 | g/a | g | R |
| 4903051 | 115890 | 14 | 71182040 | c/g | g | S |
| 11158974 | 117279 | 14 | 71183429 | t/c | c | Y |
| 1035021 | 117522 | 14 | 71183672 | c/a | t | K |
| 6574093 | 118721 | 14 | 71184871 | t/g | g | K |
| 4899439 | 119926 | 14 | 71186076 | t/c | c | Y |
| 7155287 | 119974 | 14 | 71186124 | t/a | t | W |
| 7154402 | 120281 | 14 | 71186431 | a/g | a | R |
| 7154781 | 120484 | 14 | 71186634 | g/a | a | R |
| 6574094 | 122397 | 14 | 71188547 | c/t | t | Y |
| 10132242 | 122783 | 14 | 71188933 | g/a | a | R |
| 10146959 | 123029 | 14 | 71189179 | t/c | t | Y |
| 10147466 | 123535 | 14 | 71189685 | t/c | t | Y |
| 11626411 | 124107 | 14 | 71190257 | g/c | g | S |
| 4899440 | 124427 | 14 | 71190577 | c/t | c | Y |
| 10144553 | 124738 | 14 | 71190888 | t/c | c | Y |
| 11627663 | 125329 | 14 | 71191479 | a/c | c | M |
| 11627799 | 125796 | 14 | 71191946 | a/g | a | R |
| 11851733 | 126265 | 14 | 71192415 | g/a | g | R |
| 10140317 | 126768 | 14 | 71192918 | a/t | t | W |
| 8007375 | 127306 | 14 | 71193456 | c/t | t | Y |
| 4899441 | 127544 | 14 | 71193694 | g/a | a | R |
| 11158975 | 127809 | 14 | 71193959 | a/c | c | M |
| 11419607 | 127817 | 14 | 71193967 | a/— | a | N |
| 2109793 | 129771 | 14 | 71195921 | c/g | g | S |
| 8023156 | 130061 | 14 | 71196211 | g/c | g | S |
| 10140579 | 130333 | 14 | 71196483 | g/a | a | R |
| 7148199 | 131734 | 14 | 71197884 | t/c | t | Y |
| 7146916 | 131787 | 14 | 71197937 | g/a | a | R |
| 1990439 | 132633 | 14 | 71198783 | g/a | c | Y |
| 6574095 | 135279 | 14 | 71201429 | c/g | c | S |
| 10140037 | 135709 | 14 | 71201859 | t/a | a | W |
| 8007249 | 136208 | 14 | 71202358 | a/g | g | R |
| 1990438 | 137091 | 14 | 71203241 | a/g | c | Y |
| 1990437 | 137499 | 14 | 71203649 | t/c | a | R |
| 10146484 | 137705 | 14 | 71203855 | a/g | a | R |
| 11627333 | 137726 | 14 | 71203876 | a/c | c | M |
| 7155802 | 137944 | 14 | 71204094 | g/a | a | R |
| 2109792 | 138175 | 14 | 71204325 | t/g | c | M |
| 11429363 | 138224 | 14 | 71204374 | a/— | c | N |
| 8004210 | 139319 | 14 | 71205469 | a/g | g | R |
| 2098194 | 139409 | 14 | 71205559 | c/t | g | R |
| 2109791 | 139597 | 14 | 71205747 | t/g | c | M |
| 11381729 | 140476 | 14 | 71206626 | —/a | a | N |
| 1160094 | 140808 | 14 | 71206958 | c/t | g | R |
| 11628051 | 141363 | 14 | 71207513 | a/t | t | W |
| 10136771 | 141655 | 14 | 71207805 | g/a | g | R |
| 10139903 | 142715 | 14 | 71208865 | a/c | c | M |
| 4903053 | 143253 | 14 | 71209403 | c/t | c | Y |
| 3742836 | 143911 | 14 | 71210061 | t/c | a | R |
| 3742835 | 144044 | 14 | 71210194 | g/a | c | Y |
| 8003233 | 144670 | 14 | 71210820 | t/g | g | K |
| 11621588 | 144961 | 14 | 71211111 | c/t | c | Y |
| 740981 | 146613 | 14 | 71212763 | g/c | g | S |
| 8019492 | 147413 | 14 | 71213563 | c/a | a | M |
| 8019644 | 147464 | 14 | 71213614 | g/a | a | R |
| 8019646 | 147468 | 14 | 71213618 | g/a | a | R |
| 8019653 | 147479 | 14 | 71213629 | a/g | a | R |
| 11450794 | 147488 | 14 | 71213638 | a/— | g | N |
| 11348193 | 147500 | 14 | 71213650 | —/a | a | N |
| 11450793 | 147527 | 14 | 71213677 | g/— | a | N |
| 11282735 | 147534 | 14 | 71213684 | aagagaagag/— | g | N |
| | | | | (SEQ ID NO: 4935) | | |
| 2191823 | 149573 | 14 | 71215723 | g/t | a | M |
| 12232220 | 150798 | 14 | 71216948 | a/g | g | R |
| 11158976 | 154169 | 14 | 71220319 | c/t | c | Y |
| 4903054 | 155056 | 14 | 71221206 | c/t | c | Y |
| 11846755 | 155494 | 14 | 71221644 | t/c | t | Y |
| 12431988 | 157756 | 14 | 71223906 | g/a | g | R |
| 12100526 | 157830 | 14 | 71223980 | t/c | c | Y |
| 11622568 | 158379 | 14 | 71224529 | a/c | c | M |
| 9671230 | 159906 | 14 | 71226056 | c/t | t | Y |
| 12147487 | 159951 | 14 | 71226101 | a/t | a | W |
| 4903055 | 160135 | 14 | 71226285 | t/c | t | Y |
| 12147515 | 160209 | 14 | 71226359 | g/a | g | R |
| 11625092 | 160351 | 14 | 71226501 | c/t | c | Y |
| 4899442 | 160835 | 14 | 71226985 | t/g | t | K |
| 2052146 | 161110 | 14 | 71227260 | a/c | g | K |
| 10220553 | 161346 | 14 | 71227496 | a/t | a | W |
| 10220565 | 161670 | 14 | 71227820 | a/g | g | R |
| 2332917 | 163447 | 14 | 71229597 | g/c | c | S |
| 7158910 | 163479 | 14 | 71229629 | g/a | g | R |
| 8015749 | 164377 | 14 | 71230527 | a/g | a | R |
| 8006812 | 166328 | 14 | 71232478 | a/g | a | R |
| 8008338 | 166361 | 14 | 71232511 | c/t | t | Y |
| 2052145 | 167003 | 14 | 71233153 | t/g | a | M |
| 2052144 | 167036 | 14 | 71233186 | t/g | c | M |
| 10146692 | 167759 | 14 | 71233909 | a/g | g | R |
| 11627305 | 167829 | 14 | 71233979 | a/g | a | R |
| 2079989 | 168360 | 14 | 71234510 | c/t | a | R |
| 8018881 | 168582 | 14 | 71234732 | c/a | c | M |
| 4561387 | 169462 | 14 | 71235612 | a/t | a | W |
| 11622025 | 170317 | 14 | 71236467 | a/c | c | M |
| 740980 | 170669 | 14 | 71236819 | a/g | g | R |
| 12433250 | 171295 | 14 | 71237445 | t/c | c | Y |
| 5809590 | 171395 | 14 | 71237545 | —/a | a | N |
| 758915 | 171431 | 14 | 71237581 | t/c | a | R |
| 758914 | 171626 | 14 | 71237776 | a/t | t | W |
| 7154229 | 171735 | 14 | 71237885 | g/a | g | R |
| 12437188 | 171972 | 14 | 71238122 | t/c | t | Y |
| 7159108 | 172258 | 14 | 71238408 | t/a | a | W |
| 4903058 | 172448 | 14 | 71238598 | g/c | c | S |
| 4899443 | 172918 | 14 | 71239068 | c/a | a | M |
| 11848907 | 173415 | 14 | 71239565 | a/g | g | R |
| 12432148 | 173467 | 14 | 71239617 | g/t | t | K |
| 12435078 | 173697 | 14 | 71239847 | c/t | c | Y |
| 6574096 | 173991 | 14 | 71240141 | a/g | a | R |
| 11158977 | 174091 | 14 | 71240241 | t/a | a | W |
| 11629434 | 174571 | 14 | 71240721 | g/t | g | K |
| 4378563 | 177780 | 14 | 71243930 | c/t | c | Y |
| 2098195 | 178129 | 14 | 71244279 | c/g | c | S |
| 4633653 | 178693 | 14 | 71244843 | c/a | c | M |
| 7148350 | 179325 | 14 | 71245475 | g/a | g | R |
| 740979 | 179511 | 14 | 71245661 | a/t | a | W |
| 740978 | 179608 | 14 | 71245758 | g/c | g | S |
| 740977 | 179644 | 14 | 71245794 | a/g | t | Y |
| 740976 | 179808 | 14 | 71245958 | t/c | a | R |
| 4903059 | 180041 | 14 | 71246191 | c/t | t | Y |
| 4903060 | 181420 | 14 | 71247570 | g/a | a | R |
| 11852056 | 181423 | 14 | 71247573 | a/g | g | R |
| 9635257 | 181932 | 14 | 71248082 | c/t | c | Y |
| 9635258 | 181992 | 14 | 71248142 | c/t | c | Y |
| 9635259 | 182248 | 14 | 71248398 | a/g | a | R |
| 4899444 | 182335 | 14 | 71248485 | g/a | a | R |
| 12435382 | 184268 | 14 | 71250418 | g/a | g | R |
| 12435412 | 184366 | 14 | 71250516 | g/a | g | R |
| 11627381 | 184855 | 14 | 71251005 | g/a | a | R |
| 2052143 | 185532 | 14 | 71251682 | g/a | t | Y |
| 2052142 | 185633 | 14 | 71251783 | g/a | t | Y |
| 2052141 | 185717 | 14 | 71251867 | c/t | a | R |
| 8021035 | 186637 | 14 | 71252787 | a/g | g | R |
| 11158978 | 187010 | 14 | 71253160 | a/g | a | R |
| 11621216 | 187367 | 14 | 71253517 | a/t | a | W |
| 10130873 | 187524 | 14 | 71253674 | t/c | c | Y |
| 11621358 | 187525 | 14 | 71253675 | g/a | g | R |
| 758913 | 188352 | 14 | 71254502 | a/g | t | Y |
| 11622602 | 188360 | 14 | 71254510 | a/g | g | R |
| 10136931 | 189032 | 14 | 71255182 | c/t | c | Y |
| 740975 | 189100 | 14 | 71255250 | t/g | a | M |
| 747987 | 189444 | 14 | 71255594 | t/c | c | Y |
| 11623819 | 189450 | 14 | 71255600 | c/t | c | Y |
| 12434445 | 190431 | 14 | 71256581 | c/a | a | M |
| 8003375 | 190813 | 14 | 71256963 | t/a | a | W |
| 10143596 | 190983 | 14 | 71257133 | a/g | g | R |
| 12435321 | 191493 | 14 | 71257643 | t/c | c | Y |
| 12435401 | 191588 | 14 | 71257738 | a/g | g | R |
| 8010609 | 191764 | 14 | 71257914 | t/c | t | Y |
| 11158979 | 191811 | 14 | 71257961 | g/c | c | S |
| 11158980 | 192126 | 14 | 71258276 | c/t | t | Y |
| 11627524 | 192356 | 14 | 71258506 | c/a | c | M |
| 1126160 | 192953 | 14 | 71259103 | a/c | t | K |
| 10136074 | 194133 | 14 | 71260283 | g/c | c | S |
| 4903061 | 195333 | 14 | 71261483 | t/a | a | W |
| 10665398 | 195657 | 14 | 71261807 | —/ca | a | N |
| 6574097 | 195668 | 14 | 71261818 | a/c | a | M |
| 9671654 | 195676 | 14 | 71261826 | c/g | g | S |
| 10140024 | 195805 | 14 | 71261955 | c/t | c | Y |
| 10142421 | 195998 | 14 | 71262148 | g/a | a | R |
| 10142593 | 196066 | 14 | 71262216 | c/t | c | Y |
| 11423372 | 196415 | 14 | 71262565 | —/c | a | N |
| 5809592 | 196417 | 14 | 71262567 | —/c | c | N |
| 2332918 | 196538 | 14 | 71262688 | c/t | t | Y |
| 2332919 | 196569 | 14 | 71262719 | t/c | c | Y |
| 8007348 | 196621 | 14 | 71262771 | a/c | c | M |
| 1990443 | 196806 | 14 | 71262956 | g/a | c | Y |
| 8007690 | 196823 | 14 | 71262973 | c/g | c | S |
| 3937455 | 197204 | 14 | 71263354 | g/c | g | S |
| 8012823 | 197435 | 14 | 71263585 | c/t | c | Y |
| 4899445 | 197612 | 14 | 71263762 | g/a | a | R |
| 4903062 | 197854 | 14 | 71264004 | c/t | t | Y |
| 973963 | 198264 | 14 | 71264414 | g/a | t | Y |
| 10149962 | 198835 | 14 | 71264985 | t/g | g | K |
| 8003449 | 199275 | 14 | 71265425 | a/g | a | R |
| 11620775 | 199336 | 14 | 71265486 | c/t | c | Y |
| 8009089 | 199903 | 14 | 71266053 | a/g | g | R |
| 8008967 | 200017 | 14 | 71266167 | t/c | c | Y |
| 12050132 | 200141 | 14 | 71266291 | a/t | a | W |
| 10133018 | 200552 | 14 | 71266702 | t/c | c | Y |
| 1990442 | 200868 | 14 | 71267018 | t/c | a | R |
| 1990441 | 200983 | 14 | 71267133 | t/g | c | M |
| 8015833 | 200990 | 14 | 71267140 | c/t | t | Y |
| 1990440 | 201045 | 14 | 71267195 | g/c | g | S |
| 4903063 | 202426 | 14 | 71268576 | c/g | g | S |
| 10139810 | 202841 | 14 | 71268991 | c/a | c | M |
| 4903064 | 203311 | 14 | 71269461 | t/c | t | Y |
| 2159715 | 203543 | 14 | 71269693 | a/c | g | K |
| 2109795 | 203749 | 14 | 71269899 | a/g | t | Y |
| 2159714 | 204040 | 14 | 71270190 | g/a | c | Y |
| 11623107 | 204269 | 14 | 71270419 | a/c | c | M |
| 7152005 | 204309 | 14 | 71270459 | c/t | c | Y |
| 7152489 | 204426 | 14 | 71270576 | a/g | g | R |
| 4479167 | 205135 | 14 | 71271285 | g/c | g | S |
| 4606655 | 205269 | 14 | 71271419 | t/c | c | Y |
| 4606656 | 205323 | 14 | 71271473 | t/c | c | Y |
| 4489943 | 205364 | 14 | 71271514 | a/g | g | R |
| 7142642 | 206310 | 14 | 71272460 | g/c | c | S |
| 8006233 | 206600 | 14 | 71272750 | a/g | g | R |
| 8007559 | 206786 | 14 | 71272936 | t/a | t | W |
| 8006156 | 206796 | 14 | 71272946 | a/g | a | R |
| 1468662 | 207633 | 14 | 71273783 | a/g | c | Y |
| 11627048 | 207723 | 14 | 71273873 | g/a | g | R |
| 6574098 | 208026 | 14 | 71274176 | t/c | t | Y |
| 12433760 | 208627 | 14 | 71274777 | c/t | t | Y |
| 12433780 | 208662 | 14 | 71274812 | c/t | t | Y |
| 12434506 | 209109 | 14 | 71275259 | t/a | t | W |
| 12434673 | 209742 | 14 | 71275892 | t/c | t | Y |
| 12431400 | 209750 | 14 | 71275900 | t/c | c | Y |
| 12431384 | 209817 | 14 | 71275967 | g/a | a | R |
| 4903067 | 210191 | 14 | 71276341 | c/t | t | Y |
| 2332920 | 210349 | 14 | 71276499 | g/a | a | R |
| 2215591 | 210724 | 14 | 71276874 | a/g | t | Y |
| 12433064 | 211537 | 14 | 71277687 | c/a | c | M |
| 12436263 | 211551 | 14 | 71277701 | t/g | t | K |
| 12433097 | 211659 | 14 | 71277809 | g/a | a | R |
| 12433184 | 211757 | 14 | 71277907 | g/t | g | K |
| 8015900 | 211821 | 14 | 71277971 | a/t | t | W |
| 11296981 | 212236 | 14 | 71278386 | t/— | t | N |
| 11344025 | 212237 | 14 | 71278387 | t/— | t | N |
| 11464204 | 212442 | 14 | 71278592 | —/a | t | N |
| 11441172 | 212445 | 14 | 71278595 | —/t | a | N |
| 11381807 | 212455 | 14 | 71278605 | t/— | t | N |
| 10873251 | 212730 | 14 | 71278880 | g/a | a | R |
| 2109794 | 212746 | 14 | 71278896 | t/c | g | R |
| 10873252 | 212836 | 14 | 71278986 | t/c | t | Y |
| 4520784 | 212886 | 14 | 71279036 | g/a | g | R |
| 2877820 | 213028 | 14 | 71279178 | a/t | t | W |
| 2877821 | 213029 | 14 | 71279179 | a/t | t | W |
| 4569195 | 213095 | 14 | 71279245 | c/g | c | S |
| 11628905 | 214247 | 14 | 71280397 | a/c | c | M |
| 2191822 | 214807 | 14 | 71280957 | t/c | a | R |
| 2191821 | 214921 | 14 | 71281071 | a/c | t | K |
| 11464689 | 215163 | 14 | 71281313 | t/— | t | N |
| 12431959 | 215406 | 14 | 71281556 | g/a | g | R |
| 4903068 | 216069 | 14 | 71282219 | a/g | a | R |
| 1544579 | 216849 | 14 | 71282999 | t/c | a | R |
| 7155879 | 217130 | 14 | 71283280 | g/t | g | K |
| 12433603 | 217143 | 14 | 71283293 | c/g | c | S |
| 11351432 | 217657 | 14 | 71283807 | a/— | a | N |
| 11448438 | 217725 | 14 | 71283875 | —/g | g | N |
| 4243643 | 218435 | 14 | 71284585 | g/a | g | R |
| 4140952 | 218717 | 14 | 71284867 | t/c | a | R |
| 8005372 | 218812 | 14 | 71284962 | c/t | t | Y |
| 8004109 | 218826 | 14 | 71284976 | t/c | c | Y |
| 8008398 | 218872 | 14 | 71285022 | a/c | c | M |
| 8009692 | 218890 | 14 | 71285040 | t/c | t | Y |
| 2332921 | 218944 | 14 | 71285094 | c/g | c | S |
| 12050320 | 219229 | 14 | 71285379 | a/g | g | R |
| 12050323 | 219300 | 14 | 71285450 | a/g | g | R |
| 8010316 | 219757 | 14 | 71285907 | a/g | g | R |
| 7154293 | 220533 | 14 | 71286683 | t/c | t | Y |
| 12147969 | 221534 | 14 | 71287684 | c/t | c | Y |
| 2215590 | 221632 | 14 | 71287782 | g/a | c | Y |
| 4265749 | 222234 | 14 | 71288384 | c/t | c | Y |
| 1004552 | 222241 | 14 | 71288391 | c/t | a | R |
| 4307892 | 222458 | 14 | 71288608 | g/a | g | R |
| 12431973 | 222735 | 14 | 71288885 | t/g | g | K |
| 12431975 | 222749 | 14 | 71288899 | a/g | g | R |
| 767874 | 223187 | 14 | 71289337 | t/c | t | Y |
| 11628597 | 226626 | 14 | 71292776 | g/c | c | S |
| 10129371 | 226865 | 14 | 71293015 | a/g | a | R |
| 4903069 | 227548 | 14 | 71293698 | a/c | a | M |
| 4903070 | 227690 | 14 | 71293840 | a/g | a | R |
| 4903071 | 227815 | 14 | 71293965 | g/t | g | K |
| 11622224 | 228456 | 14 | 71294606 | t/c | c | Y |
| 10459455 | 229025 | 14 | 71295175 | g/a | g | R |
| 9323577 | 229085 | 14 | 71295235 | c/t | c | Y |
| 10624648 | 231757 | 14 | 71297907 | ttg/— | c | N |
| 3058953 | 231762 | 14 | 71297912 | gtt/— | t | N |
| 11158981 | 232727 | 14 | 71298877 | t/g | g | K |
| 11158982 | 232955 | 14 | 71299105 | t/g | t | K |
| 11158983 | 233302 | 14 | 71299452 | g/t | t | K |
| 1860750 | 233629 | 14 | 71299779 | t/a | t | W |
| 1860749 | 233670 | 14 | 71299820 | g/a | c | Y |
| 1860748 | 233702 | 14 | 71299852 | a/c | t | K |
| 1860747 | 233715 | 14 | 71299865 | a/g | c | Y |
| 11419679 | 235827 | 14 | 71301977 | —/a | a | N |
| 8011777 | 236951 | 14 | 71303101 | t/c | t | Y |
| 6574099 | 237157 | 14 | 71303307 | a/g | a | R |
| 8010957 | 237317 | 14 | 71303467 | a/g | a | R |
| 11626895 | 237363 | 14 | 71303513 | g/a | g | R |
| 10151431 | 238866 | 14 | 71305016 | t/g | g | K |
| 4903072 | 239789 | 14 | 71305939 | c/a | c | M |
| 4589489 | 240170 | 14 | 71306320 | t/c | c | Y |
| 9671327 | 240430 | 14 | 71306580 | g/t | g | K |
| 4899446 | 240454 | 14 | 71306604 | c/t | t | Y |
| 10144501 | 240688 | 14 | 71306838 | t/a | t | W |
| 5809594 | 241414 | 14 | 71307564 | tg/— | g | N |
| 720317 | 241424 | 14 | 71307574 | a/g | a | R |
| 8014316 | 241741 | 14 | 71307891 | g/a | a | R |
| 11158984 | 241768 | 14 | 71307918 | t/c | c | Y |
| 6420906 | 242360 | 14 | 71308510 | c/t | c | Y |
| 8004055 | 242957 | 14 | 71309107 | a/t | a | W |
| 12434800 | 243355 | 14 | 71309505 | a/g | g | R |
| 4243644 | 243596 | 14 | 71309746 | t/a | t | W |
| 4899447 | 243648 | 14 | 71309798 | a/g | a | R |
| 4903073 | 243664 | 14 | 71309814 | g/a | a | R |
| 11848634 | 243783 | 14 | 71309933 | t/c | c | Y |
| 4899448 | 243819 | 14 | 71309969 | t/c | t | Y |
| 10131564 | 245765 | 14 | 71311915 | a/g | g | R |
| 10134150 | 246089 | 14 | 71312239 | t/g | g | K |
| 763388 | 246457 | 14 | 71312607 | a/g | c | Y |
| 11845938 | 246485 | 14 | 71312635 | a/g | g | R |
| 11845946 | 246536 | 14 | 71312686 | g/t | g | K |
| 11845915 | 246586 | 14 | 71312736 | t/c | c | Y |
| 10137463 | 247356 | 14 | 71313506 | a/c | a | M |
| 6574100 | 247573 | 14 | 71313723 | t/a | a | W |
| 7140344 | 248173 | 14 | 71314323 | a/g | g | R |
| 10646889 | 248915 | 14 | 71315065 | aa/— | a | N |
| 7146287 | 249344 | 14 | 71315494 | c/t | c | Y |
| 10605948 | 250079-250082 | 14 | 71316232 | cccc/— | c | NNNN |
| 5809595 | 250575 | 14 | 71316725 | a/— | a | N |
| 1035099 | 250701 | 14 | 71316851 | t/a | a | W |
| 7159877 | 251260 | 14 | 71317410 | t/g | t | K |
| 7142579 | 251858 | 14 | 71318008 | g/a | g | R |
| 12431762 | 251981 | 14 | 71318131 | g/a | g | R |
| 12431764 | 251990 | 14 | 71318140 | g/a | g | R |
| 10647999 | 252326 | 14 | 71318476 | agaaagaaag/— | a | N |
| | | | | (SEQ ID NO: 4936) | | |
| 6574101 | 252727 | 14 | 71318877 | c/t | c | Y |
| 6574102 | 252946 | 14 | 71319096 | t/c | c | Y |
| 1861162 | 254199 | 14 | 71320349 | t/c | g | R |
| 12432972 | 254985 | 14 | 71321135 | t/c | t | Y |
| 10146134 | 255324 | 14 | 71321474 | a/t | a | W |
| 10146616 | 255686 | 14 | 71321836 | a/c | c | M |
| 10146769 | 255718 | 14 | 71321868 | a/g | g | R |
| 8018936 | 256321 | 14 | 71322471 | g/a | g | R |
| 10483849 | 257562 | 14 | 71323712 | g/a | a | R |
| 2160137 | 258843 | 14 | 71324993 | t/c | a | R |
| 2192595 | 258880 | 14 | 71325030 | c/a | g | K |
| 2192594 | 259060 | 14 | 71325210 | a/g | c | Y |
| 11271695 | 261590 | 14 | 71327740 | tccatatggctgaggagg/— | t | N |
| | | | | (SEQ ID NO: 4937) | | |
| 10747301 | 262088 | 14 | 71328238 | g/c | c | S |
| 12050363 | 262345 | 14 | 71328495 | c/g | g | S |
| 12050368 | 262368 | 14 | 71328518 | g/a | g | R |
| 12432949 | 263858 | 14 | 71330008 | a/g | g | R |
| 7401375 | 263995 | 14 | 71330145 | t/a | a | W |
| 8019682 | 265195 | 14 | 71331345 | g/a | g | R |
| 8021248 | 265620 | 14 | 71331770 | t/c | t | Y |
| 11158985 | 265833 | 14 | 71331983 | t/a | a | W |
| 10483850 | 266387 | 14 | 71332537 | a/g | a | R |
| 8010875 | 267130 | 14 | 71333280 | a/g | g | R |
| 11626844 | 267468 | 14 | 71333618 | t/c | c | Y |
| 982972 | 267606 | 14 | 71333756 | a/g | c | Y |
| 7161198 | 268858 | 14 | 71335008 | t/c | t | Y |
| 7160347 | 269193 | 14 | 71335343 | g/a | a | R |
| 8007139 | 269991 | 14 | 71336141 | a/g | a | R |
| 8014483 | 271840 | 14 | 71337990 | c/t | c | Y |
| 12437072 | 272592 | 14 | 71338742 | t/c | t | Y |
| 8004767 | 273399 | 14 | 71339549 | c/g | c | S |
| 12588830 | 273504 | 14 | 71339654 | a/g | g | R |
| 7158449 | 274050 | 14 | 71340200 | t/c | c | Y |
| 6574103 | 274182 | 14 | 71340332 | c/t | c | Y |
| 8020478 | 274275 | 14 | 71340425 | c/t | c | Y |
| 4903077 | 276293 | 14 | 71342443 | c/t | t | Y |
| 8012359 | 276645 | 14 | 71342795 | g/a | a | R |
| 4899449 | 277166 | 14 | 71343316 | a/g | g | R |
| 10690289 | 278290 | 14 | 71344440 | aag/— | a | N |
| 6574104 | 278646 | 14 | 71344796 | g/t | t | K |
| 2110552 | 279022 | 14 | 71345172 | a/g | t | Y |
| 8008256 | 279073 | 14 | 71345223 | g/a | a | R |
| 759283 | 280198 | 14 | 71346348 | g/t | c | M |
| 4999177 | 281100 | 14 | 71347250 | a/g | g | R |
| 2333006 | 281145 | 14 | 71347295 | c/g | c | S |
| 8006178 | 282655 | 14 | 71348805 | a/c | a | M |
| 11158986 | 282836 | 14 | 71348986 | a/c | a | M |
| 7150625 | 283537 | 14 | 71349687 | c/t | t | Y |
| 8007768 | 283976 | 14 | 71350126 | t/g | t | K |
| 8007058 | 284149 | 14 | 71350299 | a/g | g | R |
| DPF3-AA | 284742 | 14 | 71350892 | c/a | a | M |
| 2215901 | 284840 | 14 | 71350990 | c/t | a | R |
| 3814866 | 284912 | 14 | 71351062 | t/g | a | M |
| 3814864 | 285091 | 14 | 71351241 | t/c | c | Y |

### Assay for Verifying and Allelotyping SNPs

The methods used to verify and allelotype the proximal SNPs of Table 41 are the same methods described in Examples 1 and 2 herein. The PCR primers and extend primers used in these assays are provided in Table 42 (SEQ ID NOS 2728-3781, respectively, in order of appearance) and Table 43 (SEQ ID NOS 3782-4608, respectively, in order of appearance), respectively.

TABLE 42

| | | |
|---|---|---|
| dbSNP rs# | Forward PCR primer | Reverse PCR primer |

| | | |
|---|---|---|
| 11846274 | ACGTTGGATGTTCACTGTGATGTGGTTGCG | ACGTTGGATGAGGAACCACGAAAGAGTGGG |
| 2526935 | ACGTTGGATGCATTGCCTCTTGCAGATGAG | ACGTTGGATGATGAAAGCTGTGTCAGACTC |
| 929328 | ACGTTGGATGCCACAGAGAAGCTATAGCTC | ACGTTGGATGAAGCAAAATGCCTGGGCTTG |
| 3742840 | ACGTTGGATGTGCTTGTCAGATCCTAGCTT | ACGTTGGATGCGGTTTATTCCTAATGCTGTC |
| 9671739 | ACGTTGGATGAGCTGCTTGTGGGTTTCTGG | ACGTTGGATGAGGGAAAAGACCACTGTGGG |
| 8006208 | ACGTTGGATGAAGCAACTTGAAGGCTAGGC | ACGTTGGATGCAATGCTCTAGGTCCTGTAG |
| 8006230 | ACGTTGGATGATTAAAACCCTTGGCAGGGC | ACGTTGGATGTGCGGTTTATTTTCCAGCCC |
| 8007836 | ACGTTGGATGAGCTTGGATGGACTCTGTTC | ACGTTGGATGGAATGGTCAGATGCTTAGGG |
| 8006889 | ACGTTGGATGGAATGGTCAGATGCTTAGGG | ACGTTGGATGAGCTTGGATGGACTCTGTTC |
| 8010823 | ACGTTGGATGGCTTTCCCAAATGCCATGTG | ACGTTGGATGTTATCTCAGAGCAGAGGAGG |
| 8011029 | ACGTTGGATGTGATAGATGCTAAGGAGTGG | ACGTTGGATGATGTTGAAAATGTGCACGGG |
| 10649920 | ACGTTGGATGCTTAGCATCTATCACGGCAG | ACGTTGGATGTTGCCCTTCCATGTCTACTC |
| 2803956 | ACGTTGGATGTTGCATCCCTTTGATAGCAG | ACGTTGGATGCCTTCGTTCACCTATGCTTC |
| 2803957 | ACGTTGGATGGGTTCATTAAGTAGGGCGTG | ACGTTGGATGAGAACAGGAATGCCCATGTG |
| 2526934 | ACGTTGGATGAGTCTGTAGGGTTGTGTTGC | ACGTTGGATGAGTGGGCCTTGTAGGAAATG |
| 9671558 | ACGTTGGATGACAGAAGCTGGAACAAAGGG | ACGTTGGATGATTTGGAAGTGAGTGGGTTC |
| 2526933 | ACGTTGGATGAGCTGTCAGGAAACTCTGAG | ACGTTGGATGGAGTTCCCTGTTCCTTCTAG |
| 2803959 | ACGTTGGATGAAGGAGGGGAGAGGAAGATG | ACGTTGGATGTCTAAGGCAGTTCCCACTCG |
| 2526932 | ACGTTGGATGCTCTTCCTGCTGTGTGTTGC | ACGTTGGATGTTCCCCTTCACCTGGTATCC |
| 2803960 | ACGTTGGATGCTCTGCAAAGTGGACAGAGC | ACGTTGGATGCCTGGTATCCAAAAGCATGC |
| 12434675 | ACGTTGGATGTTTTTAGCCTGTCGTGGAAG | ACGTTGGATGCTCTGGCCAGAGCCAACTTC |
| 4899435 | ACGTTGGATGAGAACTTTGCTGGGCTTCAC | ACGTTGGATGAACAGGATTTCCACTTCCGG |
| 2526931 | ACGTTGGATGATCCAGCATGGTCTTTGCAG | ACGTTGGATGAATAGCTACCATGGAGTGGG |
| 2803961 | ACGTTGGATGTCACATGTCTGTAAGGTGGG | ACGTTGGATGGGCCAGGATCTTTACCAATG |
| 2803962 | ACGTTGGATGGGCTAATGCACACAAGAAGG | ACGTTGGATGTAAACAAGCACTGCAGAGCC |
| 12590634 | ACGTTGGATGCTTTTAAGCTATTGTGGCAC | ACGTTGGATGCTCCAAAGGCTAATAATTGG |
| 11327712 | ACGTTGGATGAAAGCAGGCTGTCTCCAAAG | ACGTTGGATGTGGCACTTGGGAACATTTGG |
| 2803963 | ACGTTGGATGTTCCAACACACAGAGTTCTC | ACGTTGGATGCCAAACATGCAGGACTCTTG |
| 11844834 | ACGTTGGATGTGGAGATCCTAAAGCAGAGC | ACGTTGGATGTTTCCTTGCAGTTGGTCTGG |
| 11320415 | ACGTTGGATGTCATCTCCACTGCATGTCTG | ACGTTGGATGAAACTGAGGCACAGTCCAAG |
| 8012752 | ACGTTGGATGGAGGTGTTAGTCACAGAAGG | ACGTTGGATGATCCTACCCCCATTTTAGGG |
| 11623878 | ACGTTGGATGTAAGTGCTGGGATTACAGGC | ACGTTGGATGTCCTTTACGTTACATGCCTG |
| 11623926 | ACGTTGGATGACCTGTAATCCCAGCTACTC | ACGTTGGATGATCTTGGCTCACTGCAACCT |
| 12434442 | ACGTTGGATGCTGCAATCTGATCGTATCAC | ACGTTGGATGTTGTCATGCTAAAACCCGTC |
| 2526930 | ACGTTGGATGTGAAGAGAGCAGGAGAGAAG | ACGTTGGATGTAGGACCTTCACTTGCCATC |
| 12435306 | ACGTTGGATGAGGGATTTACCCTGGATATC | ACGTTGGATGATCCAGGAACCAGGAATCAC |
| 10144028 | ACGTTGGATGCTGACCATGTGAGAGGAATC | ACGTTGGATGTTTGTTGACTTGGCAGAGCC |
| 4899436 | ACGTTGGATGCTGTAAAGCCAAAGACCAGC | ACGTTGGATGTCATTCCTTCCTACAGCTGC |
| 7140593 | ACGTTGGATGTAACCAAATTCATTTGCCTC | ACGTTGGATGGTAGAGAGCTACATTTCTTTG |
| 2107687 | ACGTTGGATGAAGTGATTCCAGGTCTGGAG | ACGTTGGATGGGATCCTGAGTCCTTTTGAC |
| 991882 | ACGTTGGATGCTGCACCACAAATACTGTAC | ACGTTGGATGTCTAGCATACGTAGTTTCAG |
| 3327 | ACGTTGGATGGTTTCTGGTCCTTTGTCCTC | ACGTTGGATGTTTGGCAGCATGTACCACAG |
| 2526928 | ACGTTGGATGCATGTTAAGAAGATATCCCAC | ACGTTGGATGTGATTCTTTCCCTTTGTCAG |
| 7401336 | ACGTTGGATGCTCTAGCCCTAACAATTGAC | ACGTTGGATGCTCATGACTGTATTTGACTG |
| 2526926 | ACGTTGGATGGGCCCATACATTGCAATTAG | ACGTTGGATGTGGAGGGAAAAGTCTATGTG |
| 994760 | ACGTTGGATGAGGAAGCCAGAAACAACAGC | ACGTTGGATGGGGCATCAATGTGTTTCCAG |
| 994759 | ACGTTGGATGGACGTAGGGAAAAAAATGGG | ACGTTGGATGTCTGTTTTGCCCTTGATCTG |
| 994758 | ACGTTGGATGTCATCAACTGGTTACACCTG | ACGTTGGATGAGATCAAGGGCAAAACAGAG |
| 11848404 | ACGTTGGATGACTTCCTTCTGTAGTCCGTG | ACGTTGGATGCCTAAAGTATCCATGCCCAC |
| 2526925 | ACGTTGGATGCTTTTAGGATAGACCTGGCC | ACGTTGGATGACGGACTACAGAAGGAAGTG |
| 11844236 | ACGTTGGATGCTTCTATGTGGCATGGAAGG | ACGTTGGATGTCTGGGCAGATTCTGATCTG |
| 2526924 | ACGTTGGATGGTCAGTAATTGGCTTTGCCC | ACGTTGGATGTGGATGACATTCCCAAGTCG |
| 2803965 | ACGTTGGATGCTAATAACCCTCTCAAGGTG | ACGTTGGATGTTTCAAGAATATCAGCTGGC |
| 2526923 | ACGTTGGATGCCAGCCAGCTTAGGATAACT | ACGTTGGATGTTCTGTCTAGCACAATGGCC |
| 7152467 | ACGTTGGATGGTACATGTAAAACTAGTCAC | ACGTTGGATGAGCACAATATCAAAACCAGG |
| 11623462 | ACGTTGGATGGAGGAAGGAAGGATGAAGGG | ACGTTGGATGCCTCCTAACTAGCTTCCTGG |
| 2803966 | ACGTTGGATGTCTTCAAACCCGAAGCAAGG | ACGTTGGATGTGCTAAGGCCCATCTTAGTC |
| 2526922 | ACGTTGGATGAATGAGACTTCTTACCTGCC | ACGTTGGATGGAACCTTAGAAACAAGCTGC |
| 2526920 | ACGTTGGATGACACCAAGTTCACCACTGAC | ACGTTGGATGCCCGGATGTTCCTCTAAATG |
| 11848135 | ACGTTGGATGCGTGCATAGAAATTGGCAGC | ACGTTGGATGTTGAGTGAGAAGGCACACAG |
| 11629210 | ACGTTGGATGGTTTCATATTCACAGCTGCC | ACGTTGGATGCCTAAAAGGCTGGAAATACC |
| 2803967 | ACGTTGGATGTGATTGAATTAGGGCTCCAG | ACGTTGGATGAGACCATGCTGATTCCAAAG |
| 16455 | ACGTTGGATGTGATTCTTGCCCCACAACAG | ACGTTGGATGATTGATGTTGGGCTCTGGGC |
| 757655 | ACGTTGGATGAACAGTGTGTGAAGGTCAGG | ACGTTGGATGAGCAAATGTCAAGGCTTGGC |
| 5809581 | ACGTTGGATGTCACTGCTGTTTTCCACCAG | ACGTTGGATGCTTCCTACTGTCCTGAGTAC |
| 3057139 | ACGTTGGATGTCACTGCTGTTTTCCACCAG | ACGTTGGATGCTTCCTACTGTCCTGAGTAC |
| 2247658 | ACGTTGGATGTGAGTAGCTGGGACTACAGG | ACGTTGGATGTAACACGGTGAAACCCCGTC |
| 11625609 | ACGTTGGATGCAAGCTGGGCCTTAAAGGAT | ACGTTGGATGTGCCTTTCCCTGTAATGCTG |
| 8005580 | ACGTTGGATGGCAGGGAAAAGAGGAATTGC | ACGTTGGATGCATGCCTACATAAGAGGCTC |
| 8010472 | ACGTTGGATGTGGATTTAGTGACTGGTCCC | ACGTTGGATGGAGATGAGTCTGGTGATAGG |
| 2526919 | ACGTTGGATGAGGGAGAAGTATCTGGAGAG | ACGTTGGATGCATCTGTCACTTGATGGTGG |
| 2803968 | ACGTTGGATGGGGAACACTTCTGTCCTTTC | ACGTTGGATGTGAGAGGAGCACAAAAGACG |
| 11623943 | ACGTTGGATGATTGAAAGGAGCCAGTGAGG | ACGTTGGATGGGAAGGATCCCCTTTTTGTC |
| 11623946 | ACGTTGGATGATTGAAAGGAGCCAGTGAGG | ACGTTGGATGGGAAGGATCCCCTTTTTGTC |
| 1557926 | ACGTTGGATGAAATCGGGAGGTGGAGAATG | ACGTTGGATGACAATCGAGACACCCACAAC |
| 12434611 | ACGTTGGATGAATCTGACCTCCTTTCAGGG | ACGTTGGATGTAATGTCTGCCCTGGGTATG |
| 2526918 | ACGTTGGATGGTATGCAAACTCTAGACCAG | ACGTTGGATGCCAATCTGCATGGATAAGAC |
| 5809582 | ACGTTGGATGAGAGTGAAACCCTATCTCTG | ACGTTGGATGGGTCACATACTTCTGTCTTC |
| 8008279 | ACGTTGGATGTCAGAGATAGGGTTTCACTC | ACGTTGGATGGGTGCAGTGAACCAAGATTG |
| 2803969 | ACGTTGGATGAAAATTAGCCAGGCATGGTG | ACGTTGGATGAATCCTTCCTCCTCAGCATC |
| 2526917 | ACGTTGGATGATCCCTTCTTACCAGCTTGG | ACGTTGGATGTACAGGTATGTGCCACCATG |
| 8008774 | ACGTTGGATGAGGAATGGGTTGATGCCTTC | ACGTTGGATGCCAAGCTGGTAAGAAGGGAT |
| 2803970 | ACGTTGGATGCCAAGCTGGTAAGAAGGGAT | ACGTTGGATGAGGAATGGGTTGATGCCTTC |
| 8010255 | ACGTTGGATGCTTTTCTGTGGTATGGTGGG | ACGTTGGATGTAGCATTGCAAGCCTCTTTG |
| 2997642 | ACGTTGGATGGCCACAGCTGCCTTTTTAAG | ACGTTGGATGGTGGGATGCGGCAAATACTT |
| 2803971 | ACGTTGGATGAGTGAGAGACCTTTGTGTGC | ACGTTGGATGCCCACCATACCACAGAAAAG |
| 11313760 | ACGTTGGATGCTCACTATCTCTCAACTCCC | ACGTTGGATGATGGGCCCACTTTTGTGTAG |
| 2803972 | ACGTTGGATGTGCAGTCCCTAGGTCAGATC | ACGTTGGATGTACACTCCGGCTACACAAAA |
| 4903038 | ACGTTGGATGACAAGATCCCCAGGTGATTC | ACGTTGGATGAGAGAGTGAAGACTGAGTGG |
| 2526916 | ACGTTGGATGTTCTCTACCCTCTAACGCAG | ACGTTGGATGTTAGTGGCTGCAGAGCTGTA |
| 4903040 | ACGTTGGATGACCAAAGTCCTGGGAGGATG | ACGTTGGATGACTCATGGGCTTGGAGTCAG |
| 2803974 | ACGTTGGATGATCTGCTTACAGCTCTGCAG | ACGTTGGATGTCTGGATGCACTTCGTAAAG |
| 2803975 | ACGTTGGATGCTGATGACTACTGCTTACCC | ACGTTGGATGCTGGGATGTGTGTTTGAAGG |
| 2803976 | ACGTTGGATGGCAACCTAATTAGATTGCACC | ACGTTGGATGTTGGCTCAGGTATATTCCTC |
| 2803977 | ACGTTGGATGAGCCCCAGAACAAGCATTAC | ACGTTGGATGCTCGTGATTTTTGGCTCAGG |
| 10139909 | ACGTTGGATGTTTATTGAGGGCAGTGAGGC | ACGTTGGATGTATGGTGAGAAGTGGTACTG |
| 722945 | ACGTTGGATGTTCTTTAATCTCGCCAGGAG | ACGTTGGATGATAGGTGCATGAGCAGTGAC |
| 917069 | ACGTTGGATGGCTTGTTTGTATGCTGGCTG | ACGTTGGATGCCTGAGAATTTTGCTCCTGC |
| 2803978 | ACGTTGGATGTTATGTGGACTCACTTCCCC | ACGTTGGATGCAGAAAGAGGTTCATGCTGG |
| 2332890 | ACGTTGGATGTTGTCATGGTGGTTGGGAAC | ACGTTGGATGTGGGATGAGGATTTTCCCAC |
| 1989638 | ACGTTGGATGAGAGTGAGGACAGAGAGAAG | ACGTTGGATGCCCTTCTTCTCTGTATCCTG |
| 10679174 | ACGTTGGATGAGCGCCTCATAAAGTTGTAG | ACGTTGGATGCTCAATTGTCTCACTCATCC |
| 2526915 | ACGTTGGATGTTCCCAGTGCTTGTTTCCTG | ACGTTGGATGGGCAGAGACAGGATGAAAAC |
| 2526914 | ACGTTGGATGATAAAGGCCCTCCCTGAATG | ACGTTGGATGGATCCTCGCCTTTAACTCTG |
| 11158964 | ACGTTGGATGGAAGGAAACAGACAGCAGAG | ACGTTGGATGAGAAAAGAGACTGGATAAAG |
| 11158965 | ACGTTGGATGGGGCTGCTCCTTCTTCCTGT | ACGTTGGATGTTCTGATGTGCATTCATCCTT |
| 740081 | ACGTTGGATGACTTTCTTCTTCCATCCCCC | ACGTTGGATGAGCCTCTCTAAGAAGTGCTC |
| 740080 | ACGTTGGATGTCCCCAGATTTCTTTGCCTC | ACGTTGGATGTGGAGGTGGAATCTCCTTTG |
| 10140582 | ACGTTGGATGACGTGGCTGGCTGTGAGAC | ACGTTGGATGAAGAGGCAAAGAAATCTGGG |
| 2526913 | ACGTTGGATGGCAAGAGTGGGAAATACCTG | ACGTTGGATGATTCACGCTCATCTCTGCTC |
| 4899437 | ACGTTGGATGTCCAGAACAGAAAAGGTGCC | ACGTTGGATGAAAGGCAGTGGTGATTGCTG |
| 10145678 | ACGTTGGATGTCTGCTTCAGAAAAGAAGTG | ACGTTGGATGGGTACAATCCTTGCTACTTC |
| 2107686 | ACGTTGGATGGAGGTCATTCAACCAAAATC | ACGTTGGATGACATCATCTTTGGTGCATGG |
| 2107685 | ACGTTGGATGTGTCTTGCCACTGCATTCAC | ACGTTGGATGCTGAAGCAATGTCTCAAGCC |
| 2107684 | ACGTTGGATGGTCAACAAAAGGCTTGAGAC | ACGTTGGATGGGAGAAGATTAGTTTCACGG |
| 7154188 | ACGTTGGATGCCCCAACTCTATGCAAGAGA | ACGTTGGATGCAAGCAGTTCTTTTGACACC |
| 4903041 | ACGTTGGATGGAAAGATCAGGTAACCAAACG | ACGTTGGATGGAGGAGAAAGAGTCTTCAAAC |
| 7159637 | ACGTTGGATGCCAAGTTCTACCCTGTGATC | ACGTTGGATGGAAGACTCTTTCTCCTCCTG |
| 6574089 | ACGTTGGATGGGTTCTAGTTACATGGAGTG | ACGTTGGATGGATGACCAGTTGTCTATTAC |
| 12589753 | ACGTTGGATGAGTCAGGATACTGCTAGAGG | ACGTTGGATGGGCATGCCTTGTTCAGATTC |
| 4899438 | ACGTTGGATGTATCTTTGGGCTGCTCACAG | ACGTTGGATGCATCCTGGATCTCCTCTATC |
| 4903042 | ACGTTGGATGCCGCATTTAGCTTATCCACG | ACGTTGGATGGTCACTATAGGAAGTGGGTG |
| 4903043 | ACGTTGGATGCCGCATTTAGCTTATCCACG | ACGTTGGATGGTCACTATAGGAAGTGGGTG |
| 2803980 | ACGTTGGATGTCATTGCATGTGTAAACCGC | ACGTTGGATGTATAGGAAGTGGGTGATGTG |
| 10483848 | ACGTTGGATGAAAAAAACCCTAAGGAGGGC | ACGTTGGATGCGTTATGCCTTGCATTATGG |
| 2240344 | ACGTTGGATGAACACTGTGACTCAAGGACC | ACGTTGGATGTTTCCCTCCCTTGTAAGCAG |
| 2286068 | ACGTTGGATGAATTCAGTTGCTGGTGGGAG | ACGTTGGATGTACATCCCATCGGCCAAAAC |
| 11339669 | ACGTTGGATGATTCCTTCCTGAGCTAGACC | ACGTTGGATGAGGCAAGCCCCTTTTATCTG |
| 3854 | ACGTTGGATGCTCTAAAGAAAAATGCCCCAC | ACGTTGGATGTAAAAAGCCTGAGAGAGCAG |
| 11463 | ACGTTGGATGGAACACAAACAAACTGATGG | ACGTTGGATGACTTTTGAAGTAGGAGGTAC |
| 1383 | ACGTTGGATGAATCAGGGTAGTGTGTCCAG | ACGTTGGATGTAGCAACTGACCTGCCCTAT |
| 1381 | ACGTTGGATGTAGCAACTGACCTGCCCTAT | ACGTTGGATGAATCAGGGTAGTGTGTCCAG |
| 1377 | ACGTTGGATGAAGAGATCAGCCAGGAATAG | ACGTTGGATGTACACCACCTGGGAATTGAC |
| 11158966 | ACGTTGGATGGCAGAACTGGGTGTTAGCAA | ACGTTGGATGTAAAGGTCCTTTCTGGGGTG |
| 11158967 | ACGTTGGATGTTCCCCTCCACTTAGAAACC | ACGTTGGATGTTTCTACTTGGATGGCAGGG |
| 11158968 | ACGTTGGATGTAAAACACCCCAGAAAGGAC | ACGTTGGATGGGCTGAATTGGTTCTATACC |
| 11158969 | ACGTTGGATGTGATGTCATCTCTCTCCCTG | ACGTTGGATGCAAATGAGGCATCTGATGGG |
| 11158970 | ACGTTGGATGGGTATAGAACCAATTCAGCC | ACGTTGGATGAAGGAAGCAGTGTCCTTTTG |
| 11158971 | ACGTTGGATGTGGAACTGGTCTCTTGGTTG | ACGTTGGATGTCACAAGCACTCACACTCAG |
| 11849782 | ACGTTGGATGTAAGCTGAGCGATAAGGGAG | ACGTTGGATGGAGCTTTTTTCCTTGCTGGC |
| 10137616 | ACGTTGGATGTTATGGCACACTGGTGTCAG | ACGTTGGATGAACAGATGGCGTGTTCCTTG |
| 757572 | ACGTTGGATGTCCTATCCTTGACAGTGCTG | ACGTTGGATGACCTCGGTTCATTTGGCAAC |
| 10140566 | ACGTTGGATGTGTTGTATCCTTCTCAGTGC | ACGTTGGATGAAAGGCCAAATGTCTGAGTC |
| 2023480 | ACGTTGGATGAACAGCATCCAAGCCTGAAC | ACGTTGGATGCCATGTCCTCTTACAAGTCC |
| 2526911 | ACGTTGGATGGGTGTCTTTGATCTGGTGTG | ACGTTGGATGAATTCACACAGGGAGCTGAG |
| 4903044 | ACGTTGGATGCCTAACAGATGAGTCTGCTC | ACGTTGGATGCCCAGTATACCTCCTGTTAC |
| 11845162 | ACGTTGGATGCATCCCTTTAAGGGTTCCTG | ACGTTGGATGCCCTCCAGTGCTGTATAAAG |
| 12147210 | ACGTTGGATGTTACTGAGCTGAGGGCAAAG | ACGTTGGATGACACACCTTTCTGTTGTTGC |
| 2997643 | ACGTTGGATGTGTCCTTGGTTTCCAACCAC | ACGTTGGATGCCCAGAATAATGCCTCACAC |
| 2997644 | ACGTTGGATGAGTTAAGTTGGAGGGACTGG | ACGTTGGATGTTCTCTCTCGGAGCTTCCTT |
| 3814867 | ACGTTGGATGATCCCTGCATTTGGAAGTCG | ACGTTGGATGCGTCAGTAGAGCTGCTGTTT |
| 2286067 | ACGTTGGATGACCGGGTTAATCCCTTTGGT | ACGTTGGATGTGGGTTAAGCAACAGACAGC |
| 1060570 | ACGTTGGATGAAGCCTCTTCCCTGGAAATG | ACGTTGGATGGCACACCAAAGGGATTAACC |
| 2332891 | ACGTTGGATGGATCTGCATCCACATACATG | ACGTTGGATGTAACCGATACCTGGGCATGG |
| 2021736 | ACGTTGGATGCCCTAATAAATCAGAGCATCC | ACGTTGGATGTTATACAAGAGCAGTCCTGG |
| 7160830 | ACGTTGGATGTCAAGGGTGGTCCATTTGTG | ACGTTGGATGTGTCTTCTACGCTAGCATCC |
| 2526909 | ACGTTGGATGAAATGACCTCCCATCAGTGC | ACGTTGGATGCATTGGGAACAGTGCTCTTG |
| 7140963 | ACGTTGGATGTAGAAGCCTGGGTCTAGTTC | ACGTTGGATGGAAGTCCAGACCCATTGATC |
| 10143667 | ACGTTGGATGAATAGCACCTGTCCTTATGC | ACGTTGGATGGGAAGTATTTCTCTCCATGC |
| 2803981 | ACGTTGGATGGGGAGCTGCAATTCAAGATG | ACGTTGGATGCCTCATGACGTATGAGGATG |
| 768840 | ACGTTGGATGAAGATGTGCCTTTGCTCCTC | ACGTTGGATGTAATGGACTCACAGTTCCAC |
| 5809587 | ACGTTGGATGACTGTGCCACCCACTAACAT | ACGTTGGATGTCTAACCCGTTGTTGGAACC |
| 2023479 | ACGTTGGATGGCAGTGAATGATATCCGGTG | ACGTTGGATGGTGACTTTAGAGAGGGATGG |
| 4903045 | ACGTTGGATGGCTTTGGGCTTCGTAAATATC | ACGTTGGATGAAACACTTTGCAGCCATACC |
| 4903046 | ACGTTGGATGTCCATAAAACTACCGCCCAG | ACGTTGGATGCCTGCCAGTCATTCTTTCTG |
| 5809588 | ACGTTGGATGAGGGCCTGGTGTGCACAAAT | ACGTTGGATGTCCAGAAATGGAGCTGCTCA |
| 10220555 | ACGTTGGATGTGACTCTCAGCACCAACTTC | ACGTTGGATGAGACCTGAGGATTTGGAAGG |
| 2906132 | ACGTTGGATGGGCTTTAATTGGTGTGACAG | ACGTTGGATGATGGCATCTATCTCATAGGC |
| 11848482 | ACGTTGGATGATGTCACCCGGAAGTCATTC | ACGTTGGATGCCATATGCCTCATGGATCAC |
| 10129954 | ACGTTGGATGCACTCTCATGCTCTCAACAC | ACGTTGGATGCAGATGCAAAACACAGACCC |
| 2526921 | ACGTTGGATGAAAAGCTTGAGCCTGAAGTG | ACGTTGGATGAGAGAGGGAGAAAATGTAGG |
| 4903047 | ACGTTGGATGAAATTCCATCGACTGGGTGG | ACGTTGGATGCCTTGATCTTGGACTTCCAG |
| 11846357 | ACGTTGGATGTTAAGGGACATCGGTAGGAC | ACGTTGGATGATGTCAGGACTTTGGAACCC |
| 10136385 | ACGTTGGATGAAGCCATTGCCCTAGGATTG | ACGTTGGATGTTAGGCCCAAAATGCGAGAG |
| 12588242 | ACGTTGGATGACTGGAAGATGGCAGATTGC | ACGTTGGATGTCTACATCCTTGACCCTGTG |
| 11621517 | ACGTTGGATGAATTTCCCTCTTAGGAGCTC | ACGTTGGATGCATCTGTTTGGAGTCTTATTG |
| 2526910 | ACGTTGGATGTTAGGACTACTGCATTAGGG | ACGTTGGATGTCCTTCTCATTCTCCAATGG |
| 2526941 | ACGTTGGATGAATCCACTGCCACTCCTGAT | ACGTTGGATGTGAGAGACTGAGGGAGAAAC |
| 2803944 | ACGTTGGATGCCAGAATCCTCACAAACACC | ACGTTGGATGCCCACTAGCAAAACTGGGTT |
| 12436770 | ACGTTGGATGTCTGGAACCTTCTTCGTGTG | ACGTTGGATGGCCAAGGGTTTAAAACCTCC |
| 10220279 | ACGTTGGATGCACATATGCACATGTGCACAC | ACGTTGGATGGCGTGTGTATACGTGTTGTG |
| 10220280 | ACGTTGGATGTGTAGCCTCATTATGAGCCG | ACGTTGGATGCACAACACGTATACACACGC |
| 11844112 | ACGTTGGATGTCCTTTATTCATGCCCTGGG | ACGTTGGATGAATGAGGCTACAGGAGAACC |
| 12432455 | ACGTTGGATGATAAAGAACACACCTGTGGC | ACGTTGGATGAGAAGGCAATATTACGAGGG |
| 8015338 | ACGTTGGATGCACACATTCGAAATGCCTGC | ACGTTGGATGCCCCTCTACAACAAAACCAC |
| 3832969 | ACGTTGGATGATGTGCTGTAGTGTGTTGGC | ACGTTGGATGATTGCTAAGCCTAGGGTTGG |
| 9671972 | ACGTTGGATGGGTGGATTTAGTGTTTCACTG | ACGTTGGATGGCAGGAATAAGAAGAACTAC |
| 9671975 | ACGTTGGATGGTTTAGAACGATGTATCTGGC | ACGTTGGATGAGATGCTGAAAGGGTAGTTG |
| 7159986 | ACGTTGGATGATAGTCACTGGGTCACCAAC | ACGTTGGATGAAGAGGGACTTTGACCCTTG |
| 11848220 | ACGTTGGATGATTCTCTCTTCCTCTCTCTC | ACGTTGGATGCCTGAGTCACATGGAATTAAG |
| 5016369 | ACGTTGGATGAATGGAGTTGTCCAGAACAG | ACGTTGGATGTCACTGTTGCTTAATCACAC |
| 1986423 | ACGTTGGATGATACAGAACCATTTTGTGGG | ACGTTGGATGAGCTTAATGTGGGTAGAAGG |
| 2332909 | ACGTTGGATGCAGAACCATTTTGTGGGAAG | ACGTTGGATGGAGCTTAATGTGGGTAGAAG |
| 8013337 | ACGTTGGATGTGCACAAGTCCTCATTGTTG | ACGTTGGATGGATTGTAGGGCAGAATATGG |
| 11445680 | ACGTTGGATGTGTATGCATCTCTCCCACTG | ACGTTGGATGTTGAGCACCTGCCACAAGTG |
| 4903048 | ACGTTGGATGACTTGAAGCACTTGGAGTGG | ACGTTGGATGAATGACACCACCAGTCACAG |
| 12434793 | ACGTTGGATGGCCAGTCAAAGCAGGATTTC | ACGTTGGATGGAGGAAGAGTGAAGACTTCC |
| 2332910 | ACGTTGGATGATTGGGAAGTGGGCAAACAC | ACGTTGGATGTTGCCTTCTCTTTCTCCTTC |
| 4410017 | ACGTTGGATGTTGCTCTCGGCAAGAAGGAG | ACGTTGGATGTGTATAGCACAGTGCCCAGG |
| 4577019 | ACGTTGGATGTTGCTCTCGGCAAGAAGGAG | ACGTTGGATGTGTATAGCACAGTGCCCAGG |
| 2332911 | ACGTTGGATGTACTTCCAAAAGCCACCTGC | ACGTTGGATGAAAAGCCTGGCCAGTTGAAG |
| 4996747 | ACGTTGGATGTACTTCCAAAAGCCACCTGC | ACGTTGGATGAAGCCTGGCCAGTTGAAGTG |
| 10140820 | ACGTTGGATGTTCCTATCACAGTTGAGGAG | ACGTTGGATGAGTCCTTTGCATTCTAGCAC |
| 12431623 | ACGTTGGATGGACAATCCTAGGGACATCTC | ACGTTGGATGTCATCAGGCTTCAGGCTTTC |
| 6574090 | ACGTTGGATGTTGCAGTAGGGTTTGTGCTC | ACGTTGGATGATCACTCGCATTACCACCTG |
| 8008750 | ACGTTGGATGTTGGGTACTGTGGCTCTAAG | ACGTTGGATGAGCATGTGGGTAGGGAGATT |
| 11627420 | ACGTTGGATGGTGAGCACTGAAGGAACAGG | ACGTTGGATGTGCACTCTCTGACACAGCAG |
| 10150185 | ACGTTGGATGCTAGTTCTAGATCCCTGAGG | ACGTTGGATGAAAGCACTGCCCCTGTAAAC |
| 10150218 | ACGTTGGATGGCACACAGAAGACGACTAAG | ACGTTGGATGGTTCCTGATGTATCACTGAG |
| 9646164 | ACGTTGGATGCCTGATGTATCACTGAGTGG | ACGTTGGATGGCACACAGAAGACGACTAAG |
| 6574091 | ACGTTGGATGAGCTTGAAGCGAAAAGCACG | ACGTTGGATGCAGGGAGAGTTAGATGACTC |
| 2332912 | ACGTTGGATGTACACACCCTAAGCACCTAC | ACGTTGGATGAAGGGAAGCAGTGTAGGAAG |
| 7155359 | ACGTTGGATGCAAGCAAACTCTCTATCACC | ACGTTGGATGTTGCTCCAAGCTTCCTTTGC |
| 2332913 | ACGTTGGATGAGCCACCCATTTTGCAAAGG | ACGTTGGATGATTTAACCCTTCCTGGAATG |
| 4544184 | ACGTTGGATGCCCCTTTCTGGGTTTATGTG | ACGTTGGATGTTGGAGCAAGAACACACAGG |
| 4337224 | ACGTTGGATGATTCCCTTCCAGGTCTACAC | ACGTTGGATGTATGTCCGCTGCTCTTTGAG |
| 2332914 | ACGTTGGATGGTGCTGCCAAAAGGCATCAG | ACGTTGGATGGTAACGTCTCCACCCCAAAG |
| 4280167 | ACGTTGGATGTGACAGTAAGTATGGGCCGG | ACGTTGGATGAAGGAAGCACAGGGAAGAGG |
| 4903049 | ACGTTGGATGGGCTTCAGTCCTTTGGTTTG | ACGTTGGATGTTGCTCACCTGTATCACCTC |
| 3742837 | ACGTTGGATGAGTGAAGCTAGGAGGCTAAG | ACGTTGGATGAGCTGTACTGAAAAGCCCTC |
| 10139937 | ACGTTGGATGAGCCCTCCTCTACCAAATTC | ACGTTGGATGAGTGAAGCTAGGAGGCTAAG |
| 12434292 | ACGTTGGATGTGCTCCTTAGCCTCCTAGCT | ACGTTGGATGTACGTGCATCAGCTGGTCTG |
| 11620671 | ACGTTGGATGTCAGGACTGTCTGGCTCTTG | ACGTTGGATGTATCAGGAGCAAGGCCTGAC |
| 10140184 | ACGTTGGATGAGACAGTCCTGACATCCAGGC | ACGTTGGATGTACTGAGTAGGCCAAGCATG |
| 2332915 | ACGTTGGATGTCCTCAGCTTGTCTCTCTCC | ACGTTGGATGTCAACTCTGAGTAATGAGGG |
| 2332916 | ACGTTGGATGACATGCAAGGGACTCTTCCT | ACGTTGGATGAGGCCAAGTAGGATTCTGAG |
| 8012759 | ACGTTGGATGACTCAGAATCCTACTTGGCC | ACGTTGGATGAGGGTGTGAGTGGATGTAAG |
| 10149272 | ACGTTGGATGGATGTGTTGAGTGCTTCCTG | ACGTTGGATGGCTGTTGAGAGAGTTTGAGC |
| 11158973 | ACGTTGGATGTCCTTGAGGGCAAAAATGGC | ACGTTGGATGTGCAAAAGAGCTTTGCACGC |
| 4903050 | ACGTTGGATGAGAGGAATGAAGTAGGGTGG | ACGTTGGATGGTCTCCAGGCTCTGTTATAG |
| 10666445 | ACGTTGGATGAGGAAGAATGAAGGAGGGAG | ACGTTGGATGATGGCAGGGACTTAGCATTG |
| 2536143 | ACGTTGGATGAAGACCCTTGACTAGGACAG | ACGTTGGATGTGGGCCACACTGTTTGAGAA |
| 10638231 | ACGTTGGATGTGAGAACCACTGCTCTACAC | ACGTTGGATGATGACAGAGTGACTCCGTCT |
| 12431636 | ACGTTGGATGAGTGCAGTGGCTCAATCTCG | ACGTTGGATGCTGAGGCGGAGAATCACTTG |
| 8005904 | ACGTTGGATGCCAGGATGGTCTCAATCTCC | ACGTTGGATGACGGTGGCTCACACCTGTAAT |
| 11621058 | ACGTTGGATGTCCCAAGTAGCTGTAACCAG | ACGTTGGATGCAACATGGTGAGAACTCATC |
| 10133224 | ACGTTGGATGAGGGCTAAGGTAGGGATTAC | ACGTTGGATGAGTGGTAGAGCTGGAATTTG |
| 12433185 | ACGTTGGATGTGCACTCACAGCCTCAGTTC | ACGTTGGATGGAGGCCCCCATCTCTAAAAC |
| 740974 | ACGTTGGATGTGAAGAGGACCATTTCCCAC | ACGTTGGATGAAAGAGCTGTGCATGTCCAG |
| 740973 | ACGTTGGATGAAAGAGCTGTGCATGTCCAG | ACGTTGGATGAGGACCATTTCCCACACTTG |
| 740972 | ACGTTGGATGAAGGAGACTTTCGAGCTTGG | ACGTTGGATGAAGGAAAGCAAGTGGGTCTG |
| 740971 | ACGTTGGATGTTTGGGAGTTCACCAAAGGC | ACGTTGGATGAAACTGGCCTAGACCCATTC |
| 4243642 | ACGTTGGATGTACAGAAGAGGTATGCTGTG | ACGTTGGATGTAGTCCCTTATCATTTCCCC |
| 4415952 | ACGTTGGATGGCCTGGGAACTTGCTTCTAA | ACGTTGGATGAGGACCCCACTCAGAAATTC |
| 2215589 | ACGTTGGATGTAAGTGCTTATCCGTGGGAG | ACGTTGGATGCTGGAAAATGGACTCTCAGG |
| 4343211 | ACGTTGGATGAAGGGCATGTGACAATAACC | ACGTTGGATGCTGAGAGTCCATTTTCCAGG |
| 2536142 | ACGTTGGATGTTTTTCCCCTCTGCCCTTCC | ACGTTGGATGTCTGGAGGCTTGAGACCATC |
| 11293340 | ACGTTGGATGCTACCCCGGCAAACTTCTTT | ACGTTGGATGACCCGTACATTGTGCACATG |
| 10151620 | ACGTTGGATGTCTGAACTCTTCCAGGCTAC | ACGTTGGATGGGCCATGGTCTTACATGGAG |
| 2286840 | ACGTTGGATGAACTCCAGACAGCATTTCAG | ACGTTGGATGAGGGAAAAACCATCAGAGAG |
| 8023089 | ACGTTGGATGAAGAGACTATGCTGGTAGGG | ACGTTGGATGATTTCTGAGGTGGCCCTTTG |
| 8008009 | ACGTTGGATGATCTGGAGCACTCAAAACTG | ACGTTGGATGCCAGTCACATTGCAACATAG |
| 11419089 | ACGTTGGATGAAAGTGAGTCCCTGTCTCAG | ACGTTGGATGACAATGGGAGTAGGAAAAGA |
| 10694676 | ACGTTGGATGAAAGTGAGTCCCTGTCTCAG | ACGTTGGATGACAATGGGAGTAGGAAAAGA |
| 2536141 | ACGTTGGATGTGAGGTGGGAGGATCGCTTA | ACGTTGGATGACAGGGACTCACTTTATTGC |
| 765247 | ACGTTGGATGGAAGAGATGATGACTTGCCC | ACGTTGGATGATGCAGATGAGCTACCACAC |
| 7147626 | ACGTTGGATGTTTGCAGCTTGCCTAGCATG | ACGTTGGATGCTTCATCCTTCAGTTTCCCC |
| 10132629 | ACGTTGGATGTTTGCAGCTTGCCTAGCATG | ACGTTGGATGCTTCATCCTTCAGTTTCCCC |
| 4903051 | ACGTTGGATGGTCTAGGAAAGGGAGAACAG | ACGTTGGATGAAAACTCCCTCCTAACACCC |
| 11158974 | ACGTTGGATGTTTGTAGTTGGACACCCCAC | ACGTTGGATGAGCTTGTTTCAGACCTCAGG |
| 1035021 | ACGTTGGATGCATGGAGAGGAGATTGGATG | ACGTTGGATGATCTCTGGCCTGGAATACTG |
| 6574093 | ACGTTGGATGACATACCCAGAGCATCAAGG | ACGTTGGATGCAAGGTCTGTCATCTCTGTG |
| 4899439 | ACGTTGGATGAGAATCTCCTTCTGTTCCTC | ACGTTGGATGATGAAGAAGGTGAGGGTGAG |
| 7155287 | ACGTTGGATGAGTGTACATCCGGGGAAATG | ACGTTGGATGGAATCTCCTTCTGTTCCTCC |
| 7154402 | ACGTTGGATGGCCATGAAAAGACATGAAGG | ACGTTGGATGCCAGGATGTCATATACTTGG |
| 7154781 | ACGTTGGATGGTTCAAAGTCTGCATGACGG | ACGTTGGATGGTGGACAGATGCCATTACAC |
| 6574094 | ACGTTGGATGATGGTGACAGGAACACTCTG | ACGTTGGATGAGAAGAAGGTGGATGCCAGG |
| 10132242 | ACGTTGGATGTTGTCCGATGTCATGGCTGG | ACGTTGGATGAGCTGAGCCAGAGCAGAAAG |
| 10146959 | ACGTTGGATGTGAACTAGCTTTTCATGGAG | ACGTTGGATGTATCCCAGCTCTTGTCTGTC |
| 10147466 | ACGTTGGATGGGTGATAAAGTAAGACCCTG | ACGTTGGATGTGACAGAGGGTTGGTGTGTT |
| 11626411 | ACGTTGGATGTTGTTTCTTCCCTTGGTTCC | ACGTTGGATGAAGAACCTGGAGATAGCACC |
| 4899440 | ACGTTGGATGTACCTGCACTTTCCACTTCC | ACGTTGGATGTTTAATGGATGCCCGCTTGC |
| 10144553 | ACGTTGGATGGTCTCTTTGGACTGTAAGCC | ACGTTGGATGTAGGGACATAGGAGGTGAAC |
| 11627663 | ACGTTGGATGTCAGCTGCATTCTCATCCTG | ACGTTGGATGTTTTGTAGATGCGTCACCCG |
| 11627799 | ACGTTGGATGTGGTCCTTCCTTGAAAAGTC | ACGTTGGATGTCCTCAGTTAAAGATCTGCC |
| 11851733 | ACGTTGGATGTCTGAAGGCAGAACCTATGG | ACGTTGGATGGGTCACAGAGACATGTAGTG |
| 10140317 | ACGTTGGATGCCAGACTTTGAAGACAATGC | ACGTTGGATGAAGGAAGAAAGTCAGGTGTC |
| 8007375 | ACGTTGGATGCCCTTGCACAATGTATGTCC | ACGTTGGATGATCCTATATCCAGATGGGCC |
| 4899441 | ACGTTGGATGAATTAGAAGTAGAGGCAGAG | ACGTTGGATGGGATATAAACCCATGGATCG |
| 11158975 | ACGTTGGATGAAGCCATTGCTCTTTGCTGG | ACGTTGGATGAACCTCCGAGACAAACAAGG |
| 11419607 | ACGTTGGATGAAGCCATTGCTCTTTGCTGG | ACGTTGGATGAACCTCCGAGACAAACAAGG |
| 2109793 | ACGTTGGATGGCATGATCTTGGCTCACTGC | ACGTTGGATGTAGTCCCAGTGACTCGAGAG |
| 8023156 | ACGTTGGATGCAAGGAGAGGGAAACTGTAG | ACGTTGGATGTATGAAGTGTCTGGCACAGG |
| 10140579 | ACGTTGGATGAAGCAGGGTATCTTCCACTG | ACGTTGGATGTGGCCATTGCTCCTGGAGCTT |
| 7148199 | ACGTTGGATGTGGGTCTCTGCAACACAGTT | ACGTTGGATGATGGCACACACTTTTCCAGC |
| 7146916 | ACGTTGGATGATTCTACCTCCACACTGCAC | ACGTTGGATGGAGCTGTCTGCAGCAAATTC |
| 1990439 | ACGTTGGATGAGAGCTGAAGTCACACAGAC | ACGTTGGATGTATTACGTGCTGGAAGGGAC |
| 6574095 | ACGTTGGATGTGACCTGGTTTCCTGACACA | ACGTTGGATGTTTCAGCAACGAAGTCCAGG |
| 10140037 | ACGTTGGATGAAAAGACAGGCATCCTCAGG | ACGTTGGATGGGCTGAGTTATGTTGGAAAG |
| 8007249 | ACGTTGGATGTAGGTCAAAGAGCATGTGTG | ACGTTGGATGGGAGAGGCTCTTTTTTACAC |
| 1990438 | ACGTTGGATGCCTCTTTGGGTAAACAGACC | ACGTTGGATGTGGGAAATGACAAGCATCTG |
| 1990437 | ACGTTGGATGCCCTAGGTTCTTCCTTTCCC | ACGTTGGATGTGAGTTCACAGCCTGCTTTG |
| 10146484 | ACGTTGGATGATGGGCTCCTCAGATACATG | ACGTTGGATGGCCCATTTTAGAGGTGAGAG |
| 11627333 | ACGTTGGATGGAGCCCATTTTAGAGGTGAG | ACGTTGGATGTCCTCAGATACATGAGCAGC |
| 7155802 | ACGTTGGATGAGGAAGGACAGATTAGCTGG | ACGTTGGATGATTAACCAGAGTGTCCCATC |
| 2109792 | ACGTTGGATGTATGTTGCCCAGCCTGGTTG | ACGTTGGATGATCCCAGCATTTTGGGAGGC |
| 11429363 | ACGTTGGATGGCCAAACCATATCACCTAGC | ACGTTGGATGAACCAGGCTGGGCAACATAG |
| 8004210 | ACGTTGGATGTTCTGTTATGTGCAGCCTAG | ACGTTGGATGTCTATGTTGGCCCCTTTCAG |
| 2098194 | ACGTTGGATGTTCTGAAATCTAGGCAGAGG | ACGTTGGATGCTCAAGCCTTGACAAATTCC |
| 2109791 | ACGTTGGATGTTCTGGGATCTGGAAGATGG | ACGTTGGATGAGTGTGAAAGGGAAATGTCG |
| 11381729 | ACGTTGGATGTAGTGAGGGCTACATGTCTC | ACGTTGGATGGACAGAGTGAGACCCTGTTT |
| 1160094 | ACGTTGGATGCAGAGAATCTAGGTTGCCTG | ACGTTGGATGGAGAAGAAGCCTGGCTAAAG |
| 11628051 | ACGTTGGATGAGGTAATCAGTGGTAGCAGG | ACGTTGGATGGAGGAGGGAAAGTCAGATAG |
| 10136771 | ACGTTGGATGAAGCACTCCTTCCACCTTGG | ACGTTGGATGTAGTGAGACCCTGTCTCTAC |
| 10139903 | ACGTTGGATGATTGCAGACTGTCAAGCCTG | ACGTTGGATGAGAGCAGAGTCAGCCTAAGG |
| 4903053 | ACGTTGGATGATTGCACAACTGCACTCCAG | ACGTTGGATGGGAATTGGTGGCGTTATGTC |
| 3742836 | ACGTTGGATGAGCCGCAGTTTTGGATCTTC | ACGTTGGATGCCAGCTGTATACATACCCTG |
| 3742835 | ACGTTGGATGAAAGATTAAAGGCTCCAGGC | ACGTTGGATGTTCAGCTTCCTCTCCTTGTG |
| 8003233 | ACGTTGGATGAAAAATAGGCAGGAGGCCAG | ACGTTGGATGTGACAGTGATGCTGTTGGTG |
| 11621588 | ACGTTGGATGACCCCCCATAATAAACCAGC | ACGTTGGATGGGGCTTCTGACAAGAACAAG |
| 740981 | ACGTTGGATGGGAGGCAGATAACTCAAAGC | ACGTTGGATGTTCCCCACCTCCATCTACCA |
| 8019492 | ACGTTGGATGTCCAGCCTATATGACAGAGC | ACGTTGGATGCTCTTTCCCTCTCCCTTTCT |
| 8019644 | ACGTTGGATGCTCTTTCCCTCTCCCTTTCT | ACGTTGGATGTGACAGAGCAAGATTCTGTG |
| 8019646 | ACGTTGGATGCTCTTTCCCTCTCCCTTTCT | ACGTTGGATGTGACAGAGCAAGATTCTGTG |
| 8019653 | ACGTTGGATGTGACAGAGCAAGATTCTGTG | ACGTTGGATGCTCTTTCCCTCTCCCTTTCT |
| 11450794 | ACGTTGGATGTGACAGAGCAAGATTCTGTG | ACGTTGGATGCTCTTTCCCTCTCCCTTTCT |
| 11348193 | ACGTTGGATGCTCTTTCCCTCTCCCTTTCT | ACGTTGGATGTGACAGAGCAAGATTCTGTG |
| 11450793 | ACGTTGGATGCTCTTTCCCTCTCCCTTTCT | ACGTTGGATGTGACAGAGCAAGATTCTGTG |
| 11282735 | ACGTTGGATGCTCTTTCCCTCTCCCTTTCT | ACGTTGGATGTGACAGAGCAAGATTCTGTG |
| 2191823 | ACGTTGGATGGGCACCCAACCCAAAATTAC | ACGTTGGATGCAGTTTCAGTTCTGGGTTGG |
| 12232220 | ACGTTGGATGCAGCATCAGAAATGGTCTAG | ACGTTGGATGCCAAATTGTTGGGATTACAGG |
| 11158976 | ACGTTGGATGCCATCTGCAAGAAAATAAAC | ACGTTGGATGGGTTTCACATTTAGGTCTATG |
| 4903054 | ACGTTGGATGGCTGTTTCAGATACATGTCC | ACGTTGGATGGCGACATTCCGCCTCAAAAA |
| 11846755 | ACGTTGGATGTGTGAGATATCGTCAGATTC | ACGTTGGATGTCAGAAGCACAGCTGACAAC |
| 12431988 | ACGTTGGATGGGTCTCCTTTCAAGCTATGC | ACGTTGGATGGGATGTCAACAGAAGCCAAG |
| 12100526 | ACGTTGGATGGCTTTGACTGAATCTTGGAC | ACGTTGGATGATAGCTTGAAAGGAGACCTG |
| 11622568 | ACGTTGGATGTCAGCTTCTTCAACCTGTGG | ACGTTGGATGAGAGGGTTGAACTGAAGAAG |
| 9671230 | ACGTTGGATGAGCTGATTTCAGTCCTGGAG | ACGTTGGATGCTTCTTGCCATCCTAATATT |
| 12147487 | ACGTTGGATGACCCCAGTAGTTTCTGATGG | ACGTTGGATGAATAGCTGATTTCAGTCCTG |
| 4903055 | ACGTTGGATGAAGCGATTCTCCTGCCTCAG | ACGTTGGATGTGGTGAAACCCCATCTCTGC |
| 12147515 | ACGTTGGATGTGGAGTGCAGTGGCATGATC | ACGTTGGATGTGAGGCAGGAGAATCGCTTG |
| 11625092 | ACGTTGGATGATCCTGAATGGCCAGAGATG | ACGTTGGATGATACCACCAGGAACTCACAG |
| 4899442 | ACGTTGGATGCTACCACTCAGGAGTTGTTC | ACGTTGGATGATTTGGTTGCGTTCCCATGG |
| 2052146 | ACGTTGGATGTTATTTGAGCTCTGATAGTG | ACGTTGGATGCCACAAAAGCACGTGATTTC |
| 10220553 | ACGTTGGATGATTTTTCTGGCTCTGCCTGG | ACGTTGGATGTGTTGAAGCAGGGCTACATG |
| 10220565 | ACGTTGGATGACAAAAACTAGCTGGGTGTG | ACGTTGGATGCCGGGTTCAAGTGATTCTCA |
| 2332917 | ACGTTGGATGACCCCAGTAAGAGGCTTGAA | ACGTTGGATGGCACAGGTTAATAAATGCCTC |
| 7158910 | ACGTTGGATGCCTACTTCTTCTTCCCACAC | ACGTTGGATGGCCAGGTAAATTCAAGCCTC |
| 8015749 | ACGTTGGATGGACTGGCTTATTTGACTTAGC | ACGTTGGATGGGAAGGAAATTCTGACATATG |
| 8006812 | ACGTTGGATGACATGACATTCAACAGCCCC | ACGTTGGATGTTCCGAATCTCTGCCAACAC |
| 8008338 | ACGTTGGATGTTAAACAGACCAGGTGTTGG | ACGTTGGATGATCAGGGAGGATGAAATCTC |
| 2052145 | ACGTTGGATGGGCTTCTCAATTCAGATGGG | ACGTTGGATGGTGCTGGCTGCCTAGTTCTA |
| 2052144 | ACGTTGGATGGGGTTGCTGGAAATCTCATG | ACGTTGGATGGGTACTTCTGTGATCCCATC |
| 10146692 | ACGTTGGATGTAAGCAGAGCTGAAGAGAAG | ACGTTGGATGGAGACAGCAGGAAAACTTGG |
| 11627305 | ACGTTGGATGAAGTTTTCCTGCTGTCTCAC | ACGTTGGATGGCAGGAAGTTAGCATATTGG |
| 2079989 | ACGTTGGATGATCATGGCAGAAGGCAAAGG | ACGTTGGATGAAAAGTGTATGGTGCCTCCC |
| 8018881 | ACGTTGGATGAGAATGGCCTAATCCAGGAG | ACGTTGGATGTGTCTCTGCCTTATATGGGC |
| 4561387 | ACGTTGGATGCCCTCATGGAGTTTATAGTC | ACGTTGGATGCCCCTTTCCTTCCACATTTT |
| 11622025 | ACGTTGGATGAGCAAGTACTGCACTTTGGG | ACGTTGGATGTAAGCTAGAACCTCTGGCAC |
| 740980 | ACGTTGGATGATGCCTGCCACATTGGGTAA | ACGTTGGATGGGTAGGGCTGTCTGTTTCAT |
| 12433250 | ACGTTGGATGTGCAGGGTAGCATGAATTTG | ACGTTGGATGTATCCTCCAAGTTCAAATCC |
| 5809590 | ACGTTGGATGATAGGTCTGATGTCAGCAGC | ACGTTGGATGGTGCCACAGAACTGACATTC |
| 758915 | ACGTTGGATGCAATGCTGCTGACATCAGAC | ACGTTGGATGCAAGAAGGGCATTTCTACCC |
| 758914 | ACGTTGGATGTGTGATCCAGACATCAGCTC | ACGTTGGATGCCCTTGTTTTAGAGGTTGGG |
| 7154229 | ACGTTGGATGCCCACAGAAACAAAACCAGC | ACGTTGGATGAGCCACCATGTTTGAATGGC |
| 12437188 | ACGTTGGATGCGGACATAACTTTCCCAAGG | ACGTTGGATGGTGGCTCTGACCTTCAAAAC |
| 7159108 | ACGTTGGATGCACATCTCGAGGTTGAAGTC | ACGTTGGATGACTGGAAGGAATCACACTGC |
| 4903058 | ACGTTGGATGCCCTAGATAAAGCAGGCTTC | ACGTTGGATGTAAATCTCTCCTCTGGAGGC |
| 4899443 | ACGTTGGATGATTGCAGTGGTGCAGTGGAG | ACGTTGGATGTAGTCCCAGCTACTCTGGAG |
| 11848907 | ACGTTGGATGAAGACAGAGGTTGCAGTGAG | ACGTTGGATGTGCCATGGCCTGATTTCCAC |
| 12432148 | ACGTTGGATGGAAGACATTGGAGGTGTTAG | ACGTTGGATGAGCGAGACTCCATCTCAAAG |
| 12435078 | ACGTTGGATGCTAAGGGTATCTCACCTCAG | ACGTTGGATGCAAGAAGATCTGGGAGAAGC |
| 6574096 | ACGTTGGATGTGGCACATAGGAAATGCTCG | ACGTTGGATGTTCAAGCTGTCAGTTGCAGG |
| 11158977 | ACGTTGGATGTAGAAGGCTGGAAGTCAGTG | ACGTTGGATGTGTTGTGTTGGGCAGAAAGG |
| 11629434 | ACGTTGGATGTCAGGACCACAAATCCCATG | ACGTTGGATGATGGGAACTGAAGGTCATCC |
| 4378563 | ACGTTGGATGTTATGGCTTCCTCTCCCTAC | ACGTTGGATGCGTGTGCATATCCTGATCAC |
| 2098195 | ACGTTGGATGCAAAGTTCTCTGCCTGAGTG | ACGTTGGATGGCTCCAGTCTCTAATCACAC |
| 4633653 | ACGTTGGATGAACTGCCTGGCCATGCTAAG | ACGTTGGATGTTACCAAGTGCATGCCCGTC |
| 7148350 | ACGTTGGATGACCCCTTTGTTGGCCTTTAC | ACGTTGGATGCTTTCTTTGCCTCTCCAGTC |
| 740979 | ACGTTGGATGAAGGGCCAAGCAATGCTTTG | ACGTTGGATGATCCTAACCAGGTCTGATGG |
| 740978 | ACGTTGGATGCTTCACATCCCTCCTCAAAG | ACGTTGGATGTAGCCACGCCATTATTGGAG |
| 740977 | ACGTTGGATGCAGCAACCCAAAGCAACACG | ACGTTGGATGTCCAGTGAGGCCTCCCTCCAA |
| 740976 | ACGTTGGATGATCAGGACTACCTGAGCAAC | ACGTTGGATGTCCTGTTTCTAAGCAGGGAG |
| 4903059 | ACGTTGGATGGGTGGTCTTGTTTCAGAGTC | ACGTTGGATGCGAATCAGTCAGCAAACCTC |
| 4903060 | ACGTTGGATGAGAATCTGGAGGGACCAATG | ACGTTGGATGAATGTCCCCAGCCTTACTTC |
| 11852056 | ACGTTGGATGAATGTCCCCAGCCTTACTTC | ACGTTGGATGAGAATCTGGAGGGACCAATG |
| 9635257 | ACGTTGGATGTGAGTGCTACATGGCCTCTG | ACGTTGGATGTTGGAGACAGAGAACAAACC |
| 9635258 | ACGTTGGATGTTCTTTGCTGCCAGGGTTTG | ACGTTGGATGGCAAACCTTCAAGCCAGCAA |
| 9635259 | ACGTTGGATGTCCTGGTGCCTCAAACAAAG | ACGTTGGATGACCTGCCACATAGTAGTTGC |
| 4899444 | ACGTTGGATGCCAGTTCATCCACTTAGTAG | ACGTTGGATGCTGGGTTGCCATTTGCATAC |
| 12435382 | ACGTTGGATGTCTGTCAATGTCTCTCTCTC | ACGTTGGATGAACATCCATCCTGAATCACC |
| 12435412 | ACGTTGGATGTATTAGGCTCCCACATCTGG | ACGTTGGATGCCCTTGCATAGATGATGCTG |
| 11627381 | ACGTTGGATGTTTTTCTGGCACTGGTGGTG | ACGTTGGATGACCTCACTATGGGTGATCAG |
| 2052143 | ACGTTGGATGTGTGTCACTTCTACCTCCAC | ACGTTGGATGCCAGTGTAATCACAAGGGTC |
| 2052142 | ACGTTGGATGACTGGACTCACCCACATAAG | ACGTTGGATGGGTATGAGGGATTTTGCAGG |
| 2052141 | ACGTTGGATGATAGAAGCGTGACCTTACCC | ACGTTGGATGCCTGCAAAATCCCTCATACC |
| 8021035 | ACGTTGGATGATGTACAAACTGAGGCTAGG | ACGTTGGATGCTTCACCTGAGAGATTTCAG |
| 11158978 | ACGTTGGATGTTAGCCAGGCATAGTGGCAT | ACGTTGGATGTTATTGTAACCTCCGCCTCC |
| 11621216 | ACGTTGGATGAGCTTGCATTTACCACTCAC | ACGTTGGATGTAGAAAGTAGACCTCTTGGG |
| 10130873 | ACGTTGGATGGGCTTGGCATTTCTTGTCAC | ACGTTGGATGTAGAGAAAAGGAGGAAGGAG |
| 11621358 | ACGTTGGATGGGCTTGGCATTTCTTGTCAC | ACGTTGGATGTAGAGAAAAGGAGGAAGGAG |
| 758913 | ACGTTGGATGAGCAACCAGTCTAGTTTTCG | ACGTTGGATGCCTAGCCAACATCCTTTTCC |
| 11622602 | ACGTTGGATGCCTAGCCAACATCCTTTTCC | ACGTTGGATGGGATCTCTAAGCAACCAGTC |
| 10136931 | ACGTTGGATGTATTCTGTATCCCAGTAGCC | ACGTTGGATGCCATTTCCTATCTTGGTTTC |
| 740975 | ACGTTGGATGCTCAGTGCCAGAAATACCAG | ACGTTGGATGGAAACCAAGATAGGAAATGG |
| 747987 | ACGTTGGATGCCTTGTGAACTTCCAACCTG | ACGTTGGATGAGGTCTGGCACTGCTAAATG |
| 11623819 | ACGTTGGATGTCATCTTGGCAACTCCTTCC | ACGTTGGATGAGTAAGCCCTGATATGCCTG |
| 12434445 | ACGTTGGATGCTCTTAGTATGGAAAGGCAG | ACGTTGGATGTTGGCATCCTTTGTAAACAG |
| 8003375 | ACGTTGGATGTAAGGATGGTTCACTGTGGC | ACGTTGGATGAGCTGCCATCTGTAGCATTC |
| 10143596 | ACGTTGGATGTACCATCCCTTATTAGATGG | ACGTTGGATGACATCCATCTGTGCATCCAT |
| 12435321 | ACGTTGGATGTCCTATATTGTCAGATGGCC | ACGTTGGATGCCTACACACAAATAGGCATG |
| 12435401 | ACGTTGGATGGTATGGTTTTGGGTGTCTGG | ACGTTGGATGTAACCACTGTGGCTTGAGAG |
| 8010609 | ACGTTGGATGTCTTCTGTTTCTGTGGCTGC | ACGTTGGATGAAAAGCTGGCCCTAAAAGGC |
| 11158979 | ACGTTGGATGATGTCCAGACACCAAAACCC | ACGTTGGATGATCTACTTGGATCTGGGCAG |
| 11158980 | ACGTTGGATGAACCACACTGTGCACTCTTC | ACGTTGGATGGGGCATGAACGATATGCAGT |
| 11627524 | ACGTTGGATGCGACAGATCAGGAAAATAGC | ACGTTGGATGGTGTTCAGGACAGCATATCC |
| 1126160 | ACGTTGGATGTGTTCTCACCCTGTTCTGTT | ACGTTGGATGGGGTTCTCTCTTGACAGATG |
| 10136074 | ACGTTGGATGTTGTCCATGTGACCTCTATG | ACGTTGGATGGGGCCATGAAAACCTAGTTC |
| 4903061 | ACGTTGGATGCATAGAGCCCTCAAAGGTAG | ACGTTGGATGAGGAGTAGTGAGAAGGAATC |
| 10665398 | ACGTTGGATGGGGCTGCACCTTGTATTTAG | ACGTTGGATGTACTGTGTAAAACGAGGGAG |
| 6574097 | ACGTTGGATGGGGCTGCACCTTGTATTTAG | ACGTTGGATGTACTGTGTAAAACGAGGGAG |
| 9671654 | ACGTTGGATGTACTGTGTAAAACGAGGGAG | ACGTTGGATGGGGCTGCACCTTGTATTTAG |
| 10140024 | ACGTTGGATGCCTGCAGCTTCTAAAACTGC | ACGTTGGATGTCCCAAAGTGCTGGGATTAC |
| 10142421 | ACGTTGGATGTGATCTCAGATCACCGCAAC | ACGTTGGATGTGTAATCCCAGCTACTCAGG |
| 10142593 | ACGTTGGATGATCTGAGATCACGCCATTGC | ACGTTGGATGGGTTTGGGCTTCCATGCAGT |
| 11423372 | ACGTTGGATGCCCATCCTACTTTTCACACC | ACGTTGGATGAGTTGAGATTGTGCCACTGC |
| 5809592 | ACGTTGGATGCCCATCCTACTTTTCACACC | ACGTTGGATGTGCAGTGAGTTGAGATTGTG |
| 2332918 | ACGTTGGATGTAGGATGGGTGTTTCCTAGC | ACGTTGGATGAACCCATGGGACCACAATTC |
| 2332919 | ACGTTGGATGATGAAGGAAGAAGCCCTGAC | ACGTTGGATGTCTGAGGGCTCTCTCTAATG |
| 8007348 | ACGTTGGATGTGAAGGGAAAGGAGGCCAAG | ACGTTGGATGTCTTCTGGGATCCAGTCAGG |
| 1990443 | ACGTTGGATGTACATGATCCTCCCCTCTAC | ACGTTGGATGCACTGGATTTGGCAAGAAGG |
| 8007690 | ACGTTGGATGTCAGCTCTTAGCCATCCTTC | ACGTTGGATGGCCACTGGATTTGGCAAGAA |
| 3937455 | ACGTTGGATGACCTCCACACTCATTACCTC | ACGTTGGATGGCAAGAATAGGTTCTTTCGC |
| 8012823 | ACGTTGGATGTTGTCGGTCTCTTCTAAGTG | ACGTTGGATGGCACCGAGTAATATGGAGTC |
| 4899445 | ACGTTGGATGAAGGTGAGAGAAAACTCACC | ACGTTGGATGCAGGAGAGTCTGCCCATTTG |
| 4903062 | ACGTTGGATGATGTCTTACTTATGCCCTTG | ACGTTGGATGTCCTAAGAAATAGGTGCTTC |
| 973963 | ACGTTGGATGACAAACCACTAAACTTTCAG | ACGTTGGATGGATTTGTTCTGGCAGGAATG |
| 10149962 | ACGTTGGATGAAATGGAGTAAGGGTCAGGG | ACGTTGGATGGGGAGCTGGTGGTGAAAAAT |
| 8003449 | ACGTTGGATGTCCAGGCCAACATTTTCAGG | ACGTTGGATGTTTGTGCTACTGGAGGGAAG |
| 11620775 | ACGTTGGATGAAACCAGCTTGTTGCTTCCC | ACGTTGGATGAGAAGAAGCTGTTCAGACCC |
| 8009089 | ACGTTGGATGACACAGACAGACACTCCCAC | ACGTTGGATGACTTCTCACTCTTTCTGGTAA |
| 8008967 | ACGTTGGATGTGACCTGCCGACTTCTCACT | ACGTTGGATGTCCATCATCTAGGACCTTGG |
| 12050132 | ACGTTGGATGTGCAGGGTTCTGAAAGGAAG | ACGTTGGATGTTATGTGCAGGCATGATGGC |
| 10133018 | ACGTTGGATGGTGCTAAGTGCTTTCCATCC | ACGTTGGATGACTGCCCATCTGAGAAATGG |
| 1990442 | ACGTTGGATGCCATCCCTCCAACTCAGAAC | ACGTTGGATGTAGGTGTGCTCCCATTTAGC |
| 1990441 | ACGTTGGATGCACACCCTGGCATGAATGTG | ACGTTGGATGTCAGAGATATGCACTGCAAG |
| 8015833 | ACGTTGGATGTCAGAGATATGCACTGCAAG | ACGTTGGATGCACACCCTGGCATGAATGTG |
| 1990440 | ACGTTGGATGCCAGGGTGTGTTCTAATACG | ACGTTGGATGAAGTCACTAACCCCACACAC |
| 4903063 | ACGTTGGATGCCATCTCACATTGAGATGGG | ACGTTGGATGCCACCAACTCTCCAAAACTC |
| 10139810 | ACGTTGGATGGCTTACCTTTTTCCCATCCC | ACGTTGGATGCAATGCTGTTACCAGGAACC |
| 4903064 | ACGTTGGATGCTGCTCTGTGTCAGCAGAAA | ACGTTGGATGATGGAGGCTTTGTAGCTAGG |
| 2159715 | ACGTTGGATGTACAGGCACTGGCGAAGAAG | ACGTTGGATGCTGCCTGCAAGTTCCCATTG |
| 2109795 | ACGTTGGATGTCCTGACTCTCTCAAAACCC | ACGTTGGATGCAAACAAGGTCCCAGCATTC |
| 2159714 | ACGTTGGATGAAAGCCCCTCTAGCAAAAGG | ACGTTGGATGAAACTCTCTCGTTGCTGTGG |
| 11623107 | ACGTTGGATGTAAAGGTTAAGTGCCCTGCC | ACGTTGGATGAGTGAGAGATAGTGTTGCAG |
| 7152005 | ACGTTGGATGACTAGGCTCCCCTGCTCACTA | ACGTTGGATGACCAACTCTGTAAGAGCAGC |
| 7152489 | ACGTTGGATGATGAGATGATGGATGCTGGG | ACGTTGGATGTCCCACACTGATTATGGTGC |
| 4479167 | ACGTTGGATGGATTACAGGTGTCAGCAACC | ACGTTGGATGGCCAGAATTACCCTGATACC |
| 4606655 | ACGTTGGATGTTCCCGGTTAAGCAGTTCTC | ACGTTGGATGGTGAAACCCCGTCTCTACTA |
| 4606656 | ACGTTGGATGTCACTGCAACATCTGCCTTC | ACGTTGGATGTTAGCTGGTCATGATGGCAG |
| 4489943 | ACGTTGGATGAAGGCAGATGTTGCAGTGAG | ACGTTGGATGTTAGCTCTACGATCCATGTC |
| 7142642 | ACGTTGGATGCCTCCAGAACTGTAAGACAG | ACGTTGGATGAAACGCATGGTATCAGTTCC |
| 8006233 | ACGTTGGATGTTCCATATTTCCAGGGTGTG | ACGTTGGATGGACCTTGGGTAAGATATTGG |
| 8007559 | ACGTTGGATGGACCGTGGATAGTCATCTCC | ACGTTGGATGCACAGGCTTAGCTGCTGGTG |
| 8006156 | ACGTTGGATGTCTCCACCTTGTCTCAGAGC | ACGTTGGATGTGCCTGGCTCCACAGGCTTA |
| 1468662 | ACGTTGGATGTCATGTAGGTTGGCTCTGAC | ACGTTGGATGGCTAGAAATCACCAGCAACC |
| 11627048 | ACGTTGGATGACAAGCTGGAGAGCTTCACT | ACGTTGGATGAAAAGCACCACTCCAATCAC |
| 6574098 | ACGTTGGATGAACCTCTGACCTCAGGTGAT | ACGTTGGATGAATTGTGGTTAATGGCTGGG |
| 12433760 | ACGTTGGATGACTTTGGGAGGCCAAGGTG | ACGTTGGATGATGTTGGCCAGGCTGGTTTC |
| 12433780 | ACGTTGGATGTTGAGGTCACGGGTTTGAAA | ACGTTGGATGCCACCATGCCCAGCTAATTT |
| 12434506 | ACGTTGGATGAGCCTGGGAAACACAGAAAG | ACGTTGGATGACTGTAACCTCGAACTCGTG |
| 12434673 | ACGTTGGATGAGCAGGTGACATTACCAGTC | ACGTTGGATGGTCATTAGGGAAATGCCCAC |
| 12431400 | ACGTTGGATGAGCAGGTGACATTACCAGTC | ACGTTGGATGGTCATTAGGGAAATGCCCAC |
| 12431384 | ACGTTGGATGGGTTGCACCATTGCATATTC | ACGTTGGATGTAATGTCACCTGCTGGTGAG |
| 4903067 | ACGTTGGATGCTGCATTGGTATCAGGACTG | ACGTTGGATGCTAGCTTATTTCACTCGGCC |
| 2332920 | ACGTTGGATGTACATATCACCACACCTGGC | ACGTTGGATGTGAGCCCAGGAGTTCAAGAC |
| 2215591 | ACGTTGGATGTCCCCAGTTTGCATCTTGAC | ACGTTGGATGATTTGTTAAAATTCATAGAAC |
| 12433064 | ACGTTGGATGTGACTTGAGACCAGGAGTTC | ACGTTGGATGACATATGTGCCACCACACTC |
| 12436263 | ACGTTGGATGAGTTCAAGACCAGCCTGGAC | ACGTTGGATGATATGTGCCACCACACTCGG |
| 12433097 | ACGTTGGATGTGATCACAGCTCACTGCAAC | ACGTTGGATGTGTGGTGGCACATATGTTTG |
| 12433184 | ACGTTGGATGTCCCAAGTAGTGTGCATGCC | ACGTTGGATGTGTGATCACGCCACTGTACT |
| 8015900 | ACGTTGGATGTGGAGTGCAGTAGTGTGACC | ACGTTGGATGACACTACTTGGGAGGCTGAG |
| 11296981 | ACGTTGGATGGAGTGAGATGTGTAGATGAC | ACGTTGGATGGCCTAATTTCACCTCTCTTG |
| 11344025 | ACGTTGGATGGAGTGAGATGTGTAGATGAC | ACGTTGGATGGCCTAATTTCACCTCTCTTG |
| 11464204 | ACGTTGGATGTAACATGGCGAGAGCTCATC | ACGTTGGATGAGCTAGGACTACAGGTACAC |
| 11441172 | ACGTTGGATGTACAGGTACACACGACAGTG | ACGTTGGATGACTAGACTGAGTAACATGGC |
| 11381807 | ACGTTGGATGACTAGACTGAGTAACATGGC | ACGTTGGATGTACAGGTACACACGACAGTG |
| 10873251 | ACGTTGGATGATCTCAGGTGATCCACCCAC | ACGTTGGATGTGGGCGCAGTGGCTTGTAAT |
| 2109794 | ACGTTGGATGGCATCTGTTAAAAACAGGCC | ACGTTGGATGACTCCTGATCTCAGGTGATC |
| 10873252 | ACGTTGGATGACTGGCTTCCAGGTTCAAGT | ACGTTGGATGTGGTGAAACCCCATCTCTAC |
| 4520784 | ACGTTGGATGAATCTTGGCTCACCACAACC | ACGTTGGATGATGCCTGTAATCCCAGCTAC |
| 2877820 | ACGTTGGATGTAGTAGCATTCCAAGTGCCC | ACGTTGGATGATAATCTATGTCCTAGATTG |
| 2877821 | ACGTTGGATGTAGTAGCATTCCAAGTGCCC | ACGTTGGATGATAATCTATGTCCTAGATTG |
| 4569195 | ACGTTGGATGGGAATGCTACTATTTGAAGGG | ACGTTGGATGTCTGTATCTTTCTGGTGATG |
| 11628905 | ACGTTGGATGAGTTACATTCCAGGGAGTGG | ACGTTGGATGCCAAGACATTTCTGCACTCG |
| 2191822 | ACGTTGGATGTGCAAAGTGGTGGAGGAAAG | ACGTTGGATGATTTTTCCCGGCATCTGACC |
| 2191821 | ACGTTGGATGTGAGATGGATCTGGAGCCAC | ACGTTGGATGGAAAGTGTCCTTAGCTTGCC |
| 11464689 | ACGTTGGATGTCGCTTCAACAAGTTGCTTG | ACGTTGGATGAGAGTGAGACTCTGTCTCAA |
| 12431959 | ACGTTGGATGACTTTGGGAGGCTGAAGTAG | ACGTTGGATGCATCACCCCTGGCTAATTTT |
| 4903068 | ACGTTGGATGATGTGAGTGCCTAATACCTG | ACGTTGGATGGCCCTGAATTGATACTGAAC |
| 1544579 | ACGTTGGATGCCTTATCTCTCTAAGACATGC | ACGTTGGATGACCATTATCATCTTCCCAGG |
| 7155879 | ACGTTGGATGCCTTGATGCATCATCTCTCC | ACGTTGGATGTTGCCTCTATCCTGATCTGG |
| 12433603 | ACGTTGGATGTTGCCTCTATCCTGATCTGG | ACGTTGGATGCCTTGATGCATCATCTCTCC |
| 11351432 | ACGTTGGATGCAAGAGCGAAACTCCATCTC | ACGTTGGATGGTGGTTTGCAGAGGCTATTG |
| 11448438 | ACGTTGGATGCAATAGCCTCTGCAAACCAC | ACGTTGGATGCGGAACATGGCACATGAAAG |
| 4243643 | ACGTTGGATGAAAGCTCTTGGGCTCAAGTG | ACGTTGGATGAATACAGTGTGTGCCAGCTG |
| 4140952 | ACGTTGGATGCTCATGTTCATGCTACAAGC | ACGTTGGATGAACAAATGGAGAAAGGAGGG |
| 8005372 | ACGTTGGATGCTCAACAAACAGTGGTTGAG | ACGTTGGATGTTCTGCTATGTTGCATGACC |
| 8004109 | ACGTTGGATGGGCAGGATCTGCTGTTTTTC | ACGTTGGATGGGTTGAGTACCTGCTATGTG |
| 8008398 | ACGTTGGATGCCCATAGCTCTATGAACCTG | ACGTTGGATGATGTGCTAAGCACCATGCTG |
| 8009692 | ACGTTGGATGTCTTCTCCTCTAGAAGATGC | ACGTTGGATGAAACAGCAGATCCTGCCTTC |
| 2332921 | ACGTTGGATGACAGGTTCATAGAGCTATGG | ACGTTGGATGCTTGTCTCAATCTTCTCCTC |
| 12050320 | ACGTTGGATGTGGGCCAGCAATCTATGCAC | ACGTTGGATGTTTGCCCTTCATTGCTCCTC |
| 12050323 | ACGTTGGATGAAGGGCAAAGGCTGAGAAAG | ACGTTGGATGTAAAGTCCAAACTCCTTGGC |
| 8010316 | ACGTTGGATGCCTCATTCACATTGATCAGC | ACGTTGGATGCCATAGGACAAAAAGTGCAC |
| 7154293 | ACGTTGGATGCGGATCACTTGAGATCAGGA | ACGTTGGATGTGCTACCACACCCAGCTAAC |
| 12147969 | ACGTTGGATGTCTCCAATCCTGCTCATGGG | ACGTTGGATGTTGATACGGCTTCCACAGTG |
| 2215590 | ACGTTGGATGATGAGAGCCTTTCTTCAGGG | ACGTTGGATGTCCAAGAAGGACAGCAGTAG |
| 4265749 | ACGTTGGATGGGGAAGGAACTTGTTCAGGA | ACGTTGGATGGCCAAAACTGAAGCCTTGAG |
| 1004552 | ACGTTGGATGGCAAAACCCAGTGCCAAAAC | ACGTTGGATGGATCATCCAAGTATGCTCCC |
| 4307892 | ACGTTGGATGGCAAGCCTTCTGAAAAGTTG | ACGTTGGATGAGGTAGGGAAAGAGGAAAAC |
| 12431973 | ACGTTGGATGAGCTACTCAGTAGGCTGAAG | ACGTTGGATGTCATAGCTCATTGCAGCCTC |
| 12431975 | ACGTTGGATGAGCTACTCAGTAGGCTGAAG | ACGTTGGATGCAGTAGTGCAATCATAGCTC |
| 767874 | ACGTTGGATGAAGGTGATAAAGTCCCCTGG | ACGTTGGATGGTCACACCATGTATCCTCTG |
| 11628597 | ACGTTGGATGAAAGCCACTTGTGTCTCCAG | ACGTTGGATGAGACCACATTCCCAGACATC |
| 10129371 | ACGTTGGATGGGTGTTCTGCAGATCATATC | ACGTTGGATGTTTATTATCCAGGGTTGCCC |
| 4903069 | ACGTTGGATGAATTGCTTGAACCTGGGAGG | ACGTTGGATGAAGGCAGAGTTTCCTTCTGT |
| 4903070 | ACGTTGGATGTTTCACCATGTTGGCCAGAC | ACGTTGGATGTAATCCCAGCACTTTGGGTG |
| 4903071 | ACGTTGGATGGCATGACAAAGTGTGGGATC | ACGTTGGATGCACCTGGCCAGAATCACTTT |
| 11622224 | ACGTTGGATGATGGCCAACTCCTTTCTTCT | ACGTTGGATGAAGATAAGCTGAGAGACCAG |
| 10459455 | ACGTTGGATGTGGCTCGATCTCGACTCATG | ACGTTGGATGGGTGGTGCACACCTGTAATC |
| 9323577 | ACGTTGGATGAGGTTGCCATGAGTCGAGAT | ACGTTGGATGTCCAGGGTGCTGTTTTTTGG |
| 10624648 | ACGTTGGATGTTCTGTTTTGCCCTCCCTTC | ACGTTGGATGTTATGAGGGAGCAGTGTGTG |
| 3058953 | ACGTTGGATGTTCTGTTTTGCCCTCCCTTC | ACGTTGGATGTTATGAGGGAGCAGTGTGTG |
| 11158981 | ACGTTGGATGAGTGAGACCCTGTCTCTAAG | ACGTTGGATGGGGTGCCAATTATCTGCTTG |
| 11158982 | ACGTTGGATGCCAAAAACTACCACATGCTC | ACGTTGGATGCATCGCTATATAGTGCTCTG |
| 11158983 | ACGTTGGATGATGCCCAGCTCACAATCATG | ACGTTGGATGTTGGGTGGTGAACATATGTG |
| 1860750 | ACGTTGGATGGTCCGCTGGATTGCTTTATC | ACGTTGGATGCAGTGTGAGACTCCATCTCA |
| 1860749 | ACGTTGGATGAACATGATTAAGGATAAAGC | ACGTTGGATGTCCCCGGAATGATACATGAC |
| 1860748 | ACGTTGGATGAAAGCAATCCAGCGGACAAG | ACGTTGGATGACTCGACTAGCTAGTCTTGG |
| 1860747 | ACGTTGGATGTTGGCATTGACAGAGGAGAG | ACGTTGGATGAAAGCAATCCAGCGGACAAG |
| 11419679 | ACGTTGGATGTACTGAGCCCAGTAGTAAGG | ACGTTGGATGCTCCCTGTGTACCTCATTTC |
| 8011777 | ACGTTGGATGTGGGCGACAGAGTGAGACTC | ACGTTGGATGCATGTTGGCCAGGCTAGTCT |
| 6574099 | ACGTTGGATGAGTAGAGACGAGGTTTCACC | ACGTTGGATGAGGTGGGCAGATCACAAGGT |
| 8010957 | ACGTTGGATGAAGTGCTGGGATTATGAGCC | ACGTTGGATGTGGGTGTGTCTCCAGTAAAG |
| 11626895 | ACGTTGGATGGGGAAGAATAAGAGCCCATC | ACGTTGGATGCCAGCCAGTAGCAGAGTTTT |
| 10151431 | ACGTTGGATGAAGTGCAATGGCGCGATCTC | ACGTTGGATGTACTTGGGAAGCTGAGGCAG |
| 4903072 | ACGTTGGATGTCTGCTTCTCTTCCTGTGTG | ACGTTGGATGAAGCACAAAACGTGGTCCAG |
| 4589489 | ACGTTGGATGAGGTGAAATGTGGACCACAC | ACGTTGGATGGGCAGTTCTCCTAGCTAAAG |
| 9671327 | ACGTTGGATGATTACAGGCATGAGCCACTG | ACGTTGGATGGGATTGCTTTCTGGTTGTGC |
| 4899446 | ACGTTGGATGGGATTGCTTTCTGGTTGTGC | ACGTTGGATGGCCTCCCAAATTGCTGAGAT |
| 10144501 | ACGTTGGATGAAGAATCGCTTGAACCCAGG | ACGTTGGATGATGGAGTTTCTCTCTGTCGC |
| 5809594 | ACGTTGGATGCATGAGCGAGTTCACATGGG | ACGTTGGATGTGGACCTAAATACTGGTGGG |
| 720317 | ACGTTGGATGTGGACCTAAATACTGGTGGG | ACGTTGGATGCATGAGCGAGTTCACATGGG |
| 8014316 | ACGTTGGATGGTTCATTTCCCGGCATTCTC | ACGTTGGATGGACCTGTGCTGAGAAACAAG |
| 11158984 | ACGTTGGATGGACATAGTTCTGACCTCACC | ACGTTGGATGTTATGAGGACCAGGTGCTAG |
| 6420906 | ACGTTGGATGCCCGAGGGATGAGTATTTTG | ACGTTGGATGTAGGAGGGCGATTACCCACA |
| 8004055 | ACGTTGGATGTTTTTCCTCCTCCCACAGAC | ACGTTGGATGGGCACCAGAGTTAAGACAAC |
| 12434800 | ACGTTGGATGCCACTATTTTTTCCCCACTC | ACGTTGGATGGCAGGCTAAAAACATTCCATG |
| 4243644 | ACGTTGGATGGGGAGTTCTCTCAATTTACC | ACGTTGGATGGGCAGAATAGTTGTTGGTAG |
| 4899447 | ACGTTGGATGCTATTCTGCCATAGCCTTGC | ACGTTGGATGTCTATGCCTCAGCAAGCATG |
| 4903073 | ACGTTGGATGTGTTCCCCAAAGCTAAGGAG | ACGTTGGATGCTATTCTGCCATAGCCTTGC |
| 11848634 | ACGTTGGATGTCACTGCTGCTCTTGACTAG | ACGTTGGATGGGACAGACAGAGAGAAAACC |
| 4899448 | ACGTTGGATGTCACTGCTGCTCTTGACTAG | ACGTTGGATGGGACAGACAGAGAGAAAACC |
| 10131564 | ACGTTGGATGTGGAGAGACCAAACTGAGAG | ACGTTGGATGCCCTGCTTAGTAAATAGGCG |
| 10134150 | ACGTTGGATGGCATGGTCTGTAGAAGAAGC | ACGTTGGATGAGACACATTCTGGGAAGGTC |
| 763388 | ACGTTGGATGTACAGGGATTGTGATGTGGG | ACGTTGGATGGGGTACTCTTAGCTGAGAAC |
| 11845938 | ACGTTGGATGATTGGCTTGAGGCATGAGTG | ACGTTGGATGAGTTTCCTGACATTCCCTGC |
| 11845946 | ACGTTGGATGTCCTCGGTAAGCAGTTGTTG | ACGTTGGATGCCACATCACAATCCCTGTAC |
| 11845915 | ACGTTGGATGCCACAGTCATTCAACACTTG | ACGTTGGATGGGAATGTCAGGAAACTTAGG |
| 10137463 | ACGTTGGATGATAGCCTGCGCAGGTGAAAA | ACGTTGGATGAGCCAACCCAAATCCAGAAG |
| 6574100 | ACGTTGGATGACACCAAGAGCTGGAGTGAT | ACGTTGGATGGTCTGTCAGGTCACTAATGG |
| 7140344 | ACGTTGGATGTGGCACCTAGTACATGCTAG | ACGTTGGATGCAGAGTGAGACTCCATCTCA |
| 10646889 | ACGTTGGATGAGAGTGGGTAAGTGCACAAG | ACGTTGGATGTGGGTGACAGAGTTAAGACT |
| 7146287 | ACGTTGGATGGGTGTCCCTTCAGGTCTAAA | ACGTTGGATGCCATGCAAATGTCCACACAG |
| 10605948 | ACGTTGGATGCTTCATTGGTTCTCTGCAGC | ACGTTGGATGCAACACGTGATTCCAGAAGG |
| 5809595 | ACGTTGGATGGTGGGCTTACACCATTTCTG | ACGTTGGATGATACCACCCAACTACCCATC |
| 1035099 | ACGTTGGATGTGGGGAGAACTTTGGTCAAC | ACGTTGGATGAAAGGGTACCCAGACTTCAC |
| 7159877 | ACGTTGGATGATCTTGGCTCACCACAAACC | ACGTTGGATGGCCTGTAATCTCAGCTACTC |
| 7142579 | ACGTTGGATGTACTACATGTCACAGTTCTG | ACGTTGGATGCGCTCACTCTGTGTATGTAT |
| 12431762 | ACGTTGGATGCAACTTAAGGAGAGCTAGGG | ACGTTGGATGTCCTCCACTGGATAACTTGC |
| 12431764 | ACGTTGGATGGGGTGAGAAACATACTCAAC | ACGTTGGATGGTATTGTATGTACTCACCGC |
| 10647999 | ACGTTGGATGGCTGTTTTTCCAAATTCTTTCC | ACGTTGGATGTCCAAATCACCTTTCTTAGC |
| 6574101 | ACGTTGGATGTAAGAATGGGAGCTCAGTGG | ACGTTGGATGACCTGGATGCAAACTGGTTG |
| 6574102 | ACGTTGGATGGATGTGACAACAAGAGAGGC | ACGTTGGATGACAGTCCTCACCATCACAAC |
| 1861162 | ACGTTGGATGACCCTCTTGCTTTCATTGGG | ACGTTGGATGCTCTATATAGGCATGTGGGC |
| 12432972 | ACGTTGGATGAAAACAAAGTCCCTGCCTTC | ACGTTGGATGTTCTGCCACAGCATTTACAC |
| 10146134 | ACGTTGGATGGCATTGCACAGTTCAGTTTC | ACGTTGGATGGTTGGCACTCAGTAATTAGC |
| 10146616 | ACGTTGGATGTGACTCAATCTCCCTCTCTC | ACGTTGGATGTTCAGGATGGAAGTGTGAGG |
| 10146769 | ACGTTGGATGACATCCAGGCAGTAGAATGG | ACGTTGGATGTGAGTGGATTCCCATGACTC |
| 8018936 | ACGTTGGATGGTCAATTCAGTGGCAAGGAC | ACGTTGGATGTAAGGAGCCCATCAATGCAG |
| 10483849 | ACGTTGGATGTGTAAGACCCTTGAGGTAGC | ACGTTGGATGGCCCACTGGTACTCATTTTC |
| 2160137 | ACGTTGGATGGAAGGAAGAAGGGCAAAGGG | ACGTTGGATGGTGATGGAGTTGGTGGGAG |
| 2192595 | ACGTTGGATGTGCAATTGAGGAGACTAGGG | ACGTTGGATGTCCCACCAACTCCATCACTT |
| 2192594 | ACGTTGGATGGGCCATGCAATCGGATTTTC | ACGTTGGATGTGTTGTTGCATATGCAGGGC |
| 11271695 | ACGTTGGATGAAGAAGTGCCTTTCACCTCC | ACGTTGGATGACATACCCAAGACTGGGAAG |
| 10747301 | ACGTTGGATGTGCCTTGCTGGATTTCAGAC | ACGTTGGATGGGTATGCAATTCCAAGTGGG |
| 12050363 | ACGTTGGATGTACCTCCACTTTGCAGGGTA | ACGTTGGATGTGGAAAAGCTGCAGAGACTC |
| 12050368 | ACGTTGGATGATGGTAGATCCACTAGCAGC | ACGTTGGATGCTGCGTACTTTCACAGTCTG |
| 12432949 | ACGTTGGATGAAACAAACAACCCCATCAAC | ACGTTGGATGTTCATGTGTCTTTTGGCTGC |
| 7401375 | ACGTTGGATGGAGATACCATCTCACACCAG | ACGTTGGATGTTTCTCCACATCCTCTCCAG |
| 8019682 | ACGTTGGATGGCAAAGGTGACTCTTGTTATG | ACGTTGGATGCTCAAGTTCAAAGTTTCACG |
| 8021248 | ACGTTGGATGGGTTTATCAGGGGTTTCAGC | ACGTTGGATGTGGCAGGAAGTGAAAGACAC |
| 11158985 | ACGTTGGATGGTTGAGATTTGGGTGAGGAC | ACGTTGGATGCACCACCACACCCAGTTAAT |
| 10483850 | ACGTTGGATGCATTTCTGGAAGGAAGTGGC | ACGTTGGATGTTATTCTGATGGTAGTGCTG |
| 8010875 | ACGTTGGATGGGCCCAGGCTTATTTCTCAT | ACGTTGGATGACACAGATCTGTTTGGCTGG |
| 11626844 | ACGTTGGATGAAAGCTTACAGGCAGCAGAG | ACGTTGGATGTTAGCGCAGCTATTCACCAC |
| 982972 | ACGTTGGATGCCTCCATGAATTGCACCTTG | ACGTTGGATGGGGACAAAGCCAGAATACTC |
| 7161198 | ACGTTGGATGGAGAGATAGAGATACCAGCG | ACGTTGGATGTCTTCCAAAAAGCCCCCAAC |
| 7160347 | ACGTTGGATGTAGCTGTGTGTGGGATTTCC | ACGTTGGATGGTGCTTTGCATCTTCTGGTC |
| 8007139 | ACGTTGGATGGGTGAGACAGAGAGAGATTC | ACGTTGGATGAGGGACAGTAGCCACATATG |
| 8014483 | ACGTTGGATGCTTCTAACAGTGAAACTGGG | ACGTTGGATGTCTCTTTGCCTCTTTCATGC |
| 12437072 | ACGTTGGATGATCAGAGGTTCCAACGTCAC | ACGTTGGATGATGAGCTGGGTAAGTGAAGG |
| 8004767 | ACGTTGGATGTATATTCTCTCCCCTCCCAC | ACGTTGGATGCGGCTTTTCCTAGGAAAACC |
| 12588830 | ACGTTGGATGTAGCACTTACTTTGCCCTGC | ACGTTGGATGGCTTCCACTCCAATAGAGAC |
| 7158449 | ACGTTGGATGTATTTTTTGGAGATAGGGTC | ACGTTGGATGTTGAGTCAAGGAGTTTGAGG |
| 6574103 | ACGTTGGATGATCTGAACCACTTCCAAGGC | ACGTTGGATGAGTAGACCAGAGAGCATTGC |
| 8020478 | ACGTTGGATGAAAGAATCTGGCCTGGCATG | ACGTTGGATGTCGGGTTTTATCATGTTGGC |
| 4903077 | ACGTTGGATGGCCTCCCAAAGTGCTAAGAT | ACGTTGGATGACTATGCCTAGGTCTCCTTC |
| 8012359 | ACGTTGGATGCATCCCGGAGAGAAACTTTG | ACGTTGGATGAATGCAGAATGGTGGTTCCC |
| 4899449 | ACGTTGGATGCTTAATGCCACTGAAAACAC | ACGTTGGATGGAGGCATTCCCTCAATTTTTC |
| 10690289 | ACGTTGGATGTAGCTTGTGATCTGGGACAC | ACGTTGGATGGCCAGAAAGTTGTTGGCTTG |
| 6574104 | ACGTTGGATGGAACAATGCTGGCACATGTC | ACGTTGGATGACCTGAGATGATCACAGTCC |
| 2110552 | ACGTTGGATGTATTGGTCCGGTTACTGGTG | ACGTTGGATGCCCTCAGTGACAGTTTTACG |
| 8008256 | ACGTTGGATGTAGTTGTTTGAGTGACTGGG | ACGTTGGATGACTGTGAAACACCAGTAACC |
| 759283 | ACGTTGGATGCGACAGAAAGAGCTAAGAGG | ACGTTGGATGTGCTGTGGCACTCAGTTTAG |
| 4999177 | ACGTTGGATGGAGCAACACCCTGTCTCAAAA | ACGTTGGATGTTGATTTGTGCATACCTGGG |
| 2333006 | ACGTTGGATGATAGCATTATGTTTCCCAGG | ACGTTGGATGTGGATTTGATAAAGGCCTGC |
| 8006178 | ACGTTGGATGGCGCAGTATCTCTAAGCTTG | ACGTTGGATGAAACACGCACCCTACTGTGA |
| 11158986 | ACGTTGGATGACCGCTGATGCTGTTTCTAC | ACGTTGGATGCAGCCCGGTTATGTGATTTC |
| 7150625 | ACGTTGGATGCGGACTTGGCGGCTTAACAG | ACGTTGGATGCTGCCTTCCTCCTCCTTTTC |
| 8007768 | ACGTTGGATGACTAAAGCCAGCTCGCTCGC | ACGTTGGATGTTTCCCCGGCAGCCCCCGGAT |
| 8007058 | ACGTTGGATGAAACTCCCCGCGAGCGATCG | ACGTTGGATGCTGGGTCCAGCGGACGGAC |
| DPF3-AA | ACGTTGGATGTGTGATTGGCCCGTCGCTTC | ACGTTGGATGGGTTGTGAATGACAGTCGCC |
| 2215901 | ACGTTGGATGAAAAGGGAGTGACCTCTGGG | ACGTTGGATGAATCGGCGCTGCTGGTAGCTA |
| 3814866 | ACGTTGGATGATAATACTCTCCCGTGCGTC | ACGTTGGATGAGAGCCAGCAGGTCTTAAAG |
| 3814864 | ACGTTGGATGAGGGTAAATCCCCGTGTTTC | ACGTTGGATGACGGCAGAGCACATGTAGTA |

TABLE 43

| dbSNP rs# | Extend Primer | Term Mix |
|---|---|---|
| 11846274 | GTGCCCTTCCCACGTGTG | ACG |
| 2526935 | GGTTTTGCCTTGGGCTGCTT | ACT |
| 929328 | AGAAGCTATAGCTCTGAGCC | ACT |
| 3742840 | CGTGCTGGCTTAGACTCTC | ACT |
| 9671739 | GGAGGTGCAACTGGCAGG | ACT |
| 8006208 | TGAAGGCTAGGCCAGCATG | ACG |
| 8006230 | CCTTGGCAGGGCATCGACT | ACT |
| 8007836 | ATGGACTCTGTTCCCTTTCC | ACG |
| 8006889 | GGCATTTGTGCTCCTCTGG | ACG |
| 8010823 | CAAATGCCATGTGGACCAGA | ACG |
| 8011029 | GGGAAGCAACAGTGGCGTA | CGT |
| 10649920 | GCAGAGGGAAGGGAGGAAA | CGT |
| 2803956 | AAGTACCCAACTATTGCCAAAT | ACT |
| 2803957 | GTGTGTGTATGTGCGTGCG | ACG |
| 2526934 | GTTGTGTTGCTGAGGGAATAT | ACT |
| 9671558 | CACAGCCAGGGCTCACTCA | ACT |
| 2526933 | TCCGGGCTTGCCAGTTCC | ACT |
| 2803959 | GGGATGTGGGAGGATAGACA | ACT |
| 2526932 | TCTTACTCCCATCCCCAAAG | ACG |
| 2803960 | AAGTGGACAGAGCTCCTCC | ACG |
| 12434675 | GGTCCCCATGTCTCAGCC | ACG |
| 4899435 | CAGAGAGGTGAGGCTGCCA | ACT |
| 2526931 | AGGTGACATTGGTAAAGATCC | ACG |
| 2803961 | GCATGGTCTTTGCAGGTATTC | ACT |
| 2803962 | AGGCGGACATGGAAAACATC | ACG |
| 12590634 | GCTATTGTGGCACTTGGGAA | ACG |
| 11327712 | TGTCTCCAAAGGCTAATAATTG | ACT |
| 2803963 | CTCCCCACCTCTTTCTTCTA | ACT |
| 11844834 | CCAGAACGCGCCCTCCTC | ACG |
| 11320415 | CCACTGCATGTCTGGGAAAA | CGT |
| 8012752 | GGCGTCTTGGAGGAGGTAC | ACT |
| 11623878 | ACAGGCATGCGCCACAGC | ACT |
| 11623926 | ATCCCAGCTACTCAGGAGG | ACG |
| 12434442 | TCTGATCGTATCACTCCCCT | ACT |
| 2526930 | GTCTGGGCCTGAGATATGGA | ACG |
| 12435306 | GATTCCCATTCTGCTGGAAC | ACG |
| 10144028 | GCCCCAACAACCTCACCC | ACT |
| 4899436 | GCCAAAGACCAGCGTCAAC | ACG |
| 7140593 | CATTTGCCTCATTTTTCTTTTGT | ACT |
| 2107687 | GGTCTGGAGCATAAAATGTATAA | ACT |
| 991882 | CAAATACTGTACTCTAGTTAATTT | CGT |
| 3327 | CTTTGTCCTCAATTTTTAACACT | ACG |
| 2526928 | GATATCCCACAATCACTATTTTTA | ACT |
| 7401336 | AAAATGTCAGACAGCACAGAG | ACT |
| 2526926 | AATATCTGTAGGTGCATATAAAC | CGT |
| 994760 | GAAACAACAGCATTAGGGCC | ACT |
| 994759 | CCCACATTCTTCACCACCC | CGT |
| 994758 | CCTGCTGGGGAATATGGTCT | ACG |
| 11848404 | GTCCGTGGTACTGTTCAGG | CGT |
| 2526925 | GCTCCATTTCTTTGCCATCC | ACG |
| 11844236 | CTTGGATGACATTCCCAAGTC | ACT |
| 2526924 | ATTGGCTTTGCCCTGGGTG | ACG |
| 2803965 | CCCTCTCAAGGTGATACCAT | ACT |
| 2526923 | TAGGATAACTATTCATTAACTTAC | ACT |
| 7152467 | AACTAGTCACATCTGAATAAAGT | ACG |
| 11623462 | AGGGGTGGGAGGGTACAG | ACT |
| 2803966 | ACAGCCTCAAGCCCCCGA | ACG |
| 2526922 | AATAATACACACTGTTAGACAAAA | ACT |
| 2526920 | CACCACTGACCCAGCTCAA | ACG |
| 11848135 | ACTAACACTAGTTTTGCCTGG | ACT |
| 11629210 | GCTGCCAATTTCTATGCACG | CGT |
| 2803967 | AAAATTATCTTCCTAGGTATGCA | ACG |
| 16455 | CAGTGTGTGAAGGTCAGGC | ACT |
| 757655 | GTCAGGCGGCTGAATCATTG | ACG |
| 5809581 | GTTTTCCACCAGCTATACATCA | CGT |
| 3057139 | GTTTTCCACCAGCTATACATCA | CGT |
| 2247658 | ACAGGCGCCCGCCACCA | ACG |
| 11625609 | CCTTAAAGGATAAATAGGAGTTTT | ACG |
| 8005580 | TTGCAGGAGATGATGCTAGAA | ACT |
| 8010472 | TCCCCAGCCATCTCCAACA | ACG |
| 2526919 | GAGGCACCTCATGACATCC | ACT |
| 2803968 | ACAGTGTAATGGAGGTGTGG | ACG |
| 11623943 | GTTGTGGGTGTCTCGATTGTA | ACT |
| 11623946 | GTGGGGTTGTGGGTGTCTC | ACT |
| 1557926 | GATTGAAAGGAGCCAGTGAG | ACT |
| 12434611 | GGATAGCTAAATATGTGGCAC | ACT |
| 2526918 | TGACAGCCACTTGGAGTGC | ACT |
| 5809582 | CCTATCTCTGAAAAAAAAAAAAAAA | CGT |
| 8008279 | GTTTCACTCTGTCACCCAGG | ACT |
| 2803969 | GCCAGGCATGGTGGCACAT | ACT |
| 2526917 | AGCATAGGGAGACCCCATC | ACT |
| 8008774 | CTGGTGGAGTAGAAAAAATATTA | ACT |
| 2803970 | GAAGGGATTTTTTAATGATGGC | ACT |
| 8010255 | CCATGGTGGGAAAAGAAGAG | ACT |
| 2997642 | TTTCCCACCATGGACCCCA | ACT |
| 2803971 | GCGTTGAGGGTGTGGGATG | ACG |
| 11313760 | TCCCATTCATGGCCAAACCT | ACT |
| 2803972 | CTAGGTCAGATCCGGGGTG | CGT |
| 4903038 | GGTGATTCGCATGCACAGG | ACT |
| 2526916 | AAGTCCTGGGAGGATGGGA | CGT |
| 4903040 | CTGCTTACAGCTCTGCAGC | ACG |
| 2803974 | CTGCAGCCACTAAGTGGAG | CGT |
| 2803975 | TCCCTGAAGACCTTTCTCCA | ACG |
| 2803976 | ATGACTGGATTCAACAGCCC | ACG |
| 2803977 | CTTTTGATCATTTTCTATTTTAGC | ACT |
| 10139909 | GGCAGTGAGGCAGACACCA | ACG |
| 722945 | GCCAGGAGCTCCAGTCCA | ACG |
| 917069 | GTATGCTGGCTGAATGGTCT | ACT |
| 2803978 | CTTCCCCTCAATGTCTTCTAT | CGT |
| 2332890 | GGTTGGGAACCCGAAGAATA | ACT |
| 1989638 | GAGAGAAGCAACAAAGTGAGAA | ACT |
| 10679174 | TACATTAATCCCCCCCCCC | CGT |
| 2526915 | GGCCGAGCGGTGTAGTGG | ACG |
| 2526914 | AAGGCCACTTCCAAACCCC | ACT |
| 11158964 | AACAGACAGCAGAGAGCCAT | ACG |
| 11158965 | CTTCCTGTTCAGCCACCAC | ACT |
| 740081 | CCCCCTGTCTTCCTGACTT | ACT |
| 740080 | TTGCCTCTTGTTACCCCATTT | ACT |
| 10140582 | CTGGCTGTGAGACTGTCAG | ACT |
| 2526913 | AGTGGGAAATACCTGAGCAAA | ACT |
| 4899437 | TGCCTTACACAGTGGAGAAG | ACT |
| 10145678 | TTCAGAAAAGAAGTGATCGGC | ACT |
| 2107686 | CTGAGAATAATATCCAAGTCCT | ACT |
| 2107685 | GCTCGTGGCTCATGCTACC | ACT |
| 2107684 | CAAAAGGCTTGAGACATTGCTT | ACG |
| 7154188 | CTTGGCTATCAGTGAGTTCC | ACT |
| 4903041 | AGGTAACCAAACGATGTGTGT | ACG |
| 7159637 | CTACCCTGTGATCACATATGAA | ACT |
| 6574089 | GGTTTCTTAGAAAAGAAGGGTAA | ACT |
| 12589753 | CCATAGATTAAGGCAAAGATCC | ACT |
| 4899438 | GATGAGAGGTCTGGCATGAA | ACT |
| 4903042 | AACACAGAGTAAAATGAAGTTTC | CGT |
| 4903043 | CCACGTATCTTTTGATGAACAC | CGT |
| 2803980 | TAAACCGCATTTAGCTTATCCA | ACG |
| 10483848 | GCTTTCTTTTTACTGCTAGGAA | CGT |
| 2240344 | GCTGACCAGAATACCCAGC | ACT |
| 2286068 | AGACGGCGGGTGTTCTAAC | ACT |
| 11339669 | CATCAGACATGATAACTAGGAG | ACT |
| 3854 | AAGAAAAATGCCCCACATTTTAA | ACT |
| 11463 | TAAAAGGAAAAAAGTGCTGACAT | ACT |
| 1383 | CGCCTTGAACTTCAGCAGCA | CGT |
| 1381 | ACCTGCCCTATTCCTGGCT | ACT |
| 1377 | CTGGCCAGAGGCAGAGAGA | ACT |
| 11158966 | AAAAAAAAATTCCCCTCCACTTA | ACT |
| 11158967 | TTTTCACAGTTAAAACACCCCA | ACT |
| 11158968 | TGATGTCATCTCTCTCCCTG | ACG |
| 11158969 | CTCTCCCTGCCATCCAAGTA | ACT |
| 11158970 | AGCCACAGAAAACCCCATCA | ACT |
| 11158971 | GGGACTGTCTCCGAGTAACT | ACG |
| 11849782 | GATAAGGGAGGGAGAAGAGG | ACT |
| 10137616 | TCAGGCACTGTCTGGAGGA | ACG |
| 757572 | GACAGTGCTGAGCAGTCACA | ACG |
| 10140566 | TCCTTCTCAGTGCATTACATC | CGT |
| 2023480 | GACTAACCAGTATCGAGGTTG | CGT |
| 2526911 | GTGGAAGCCTAGAGTGGAG | CGT |
| 4903044 | GTCTGCTCCAATTTTAAGTACA | ACG |
| 11845162 | CTTTAAGGGTTCCTGCTTACT | ACT |
| 12147210 | GCAAAGGAAGCTGGCCAGAT | ACG |
| 2997643 | ACTTTAGTGGTGGATGTGATAT | CGT |
| 2997644 | CATTGCATTGGAGGGAAATGT | CGT |
| 3814867 | GTCGTTCTTTGCCAGGAGG | ACT |
| 2286067 | GTTAATCCCTTTGGTGTGCAA | ACT |
| 1060570 | CCCAAATCCCCGTCACCAG | ACT |
| 2332891 | CACACACACACACAGACACA | ACT |
| 2021736 | AATAAATCAGAGCATCCTGAAAT | ACG |
| 7160830 | GTCCATTTGTGCAATTCTGTCA | ACT |
| 2526909 | TTCTTTGTTTTCCATTTCAGGG | ACT |
| 7140963 | GCACTTGGGGTCTTGGGCA | ACT |
| 10143667 | CATTTCCAGGGGCTGCTTGA | ACT |
| 2803981 | AAGATGAGATTTGGGTGGGG | ACT |
| 768840 | TCCTCCTTTGCCTTCTGCC | ACT |
| 5809587 | CCCTGCCATCCCTCTCTAA | ACT |
| 2023479 | GCTCCCAGGGCATCACTGT | ACT |
| 4903045 | AATATCATATTGGGCGAATCAG | ACT |
| 4903046 | CGGATCTCCGCTGTGGTGT | ACT |
| 5809588 | GCACAAATGCGCGGCCAC | ACT |
| 10220555 | GGCTGTGCTGCCCCAAGA | ACG |
| 2906132 | GGTGTGACAGTCATGTCACC | ACG |
| 11848482 | CCGGAAGTCATTCACTTAATCT | ACT |
| 10129954 | TGCTCTCAACACTTTCATCAC | ACT |
| 2526921 | TTAGGATTCATCCTTGCCTCT | ACG |
| 4903047 | GACTGGGTGGCTTAAACAAC | CGT |
| 11846357 | GCCTTGGGACTAGGGCCC | ACT |
| 10136385 | GGGCTCCACGTGTCTCTTG | ACT |
| 12588242 | CCCTGGACTGACCAACTCC | CGT |
| 11621517 | CCTCTTAGGAGCTCTGAGTA | ACG |
| 2526910 | GGGGTTTGCAAAGGCTTCAC | ACG |
| 2526941 | CCCCCAAGCTGATGCTCTC | ACT |
| 2803944 | CATCTCACAGTGCCCTCCT | ACG |
| 12436770 | GGGGAGGCCCCGGATCC | ACT |
| 10220279 | ACATGCACGCACACACACAA | CGT |
| 10220280 | GGGTGTGTGTGTGTCTATGC | ACT |
| 11844112 | CATGCCCTGGGGTTTGGAAT | ACT |
| 12432455 | CACCTGTGGCCAAGACCATT | ACT |
| 8015338 | TCGAAATGCCTGCTTGGCTA | ACG |
| 3832969 | TTCAGCCAGCCAGCCAGC | ACG |
| 9671972 | TAGTGTTTCACTGTTTAGAACG | ACT |
| 9671975 | TTAAGTAGTTCTTCTTATTCCTG | ACG |
| 7159986 | CCTTAACAAAGAAGTCCAACCA | ACT |
| 11848220 | CTTCCTCTCTCTCATAAATATTG | ACT |
| 5016369 | GTTGTCCAGAACAGCCTTCTA | ACT |
| 1986423 | GAGATGGGCGAAGAGGCAA | ACT |
| 2332909 | ATGGGCGAAGAGGCAAATTG | ACG |
| 8013337 | CAATTGAAATAGCCCCTCGC | ACG |
| 11445680 | AGGTCCTGTGTATCCGCAG | ACT |
| 4903048 | GGTAAACACTCAATAGCTGTTG | ACT |
| 12434793 | TCAAGACGTTTCTTTCCATTGT | ACT |
| 2332910 | GGGCAAACACTCCTTCTGCA | ACT |
| 4410017 | GGAGAACGGAGGGTGGGAA | ACT |
| 4577019 | GAGAACGGAGGGTGGGAAG | ACT |
| 2332911 | ACCTGCTCAGACTGGGTAC | ACT |
| 4996747 | CTGGGTACGCAGGGTACAG | ACT |
| 10140820 | TAGCTTCAGGACCTACAGAG | CGT |
| 12431623 | GGACATCTCTAGGAAGGTTG | ACT |
| 6574090 | TTTGTGCTCCTGTTAGAATCC | CGT |
| 8008750 | CTGATGTTCCCAGCAACCCT | ACT |
| 11627420 | GGGCAGGGAGTGGTCACA | ACG |
| 10150185 | TCACCACACTGACTTCCACA | ACT |
| 10150218 | ACGTACCATTTTAACGCCCC | ACT |
| 9646164 | GAGTGGGGCAGCGGGGC | ACT |
| 6574091 | CACGTGAGCCTTAAAGAATATG | ACG |
| 2332912 | CAGGAATTAAGACCATGATTCA | ACT |
| 7155359 | CTGCAGTTAGCAGCTACAATA | ACT |
| 2332913 | GAAGCTTGGAGCAAGAACACA | ACG |
| 4544184 | ATTTAACCCTTCCTGGAATGC | CGT |
| 4337224 | GGTCTACACTTAAGCATGGCA | ACG |
| 2332914 | GCATCAGACCCCATGCCC | ACG |
| 4280167 | CGGTCCCACCTTCCTTCCT | ACG |
| 4903049 | CTAGTAGAAACCACCTCTTGG | CGT |
| 3742837 | CACGGTAGTGTGTGGGAATTT | ACT |
| 10139937 | CCCACACACTACCGTGCC | ACT |
| 12434292 | TTAGCCTCCTAGCTTCACTG | ACT |
| 11620671 | GGGATGGACACAGAAGCCT | CGT |
| 10140184 | GCCCAGTGCACGAGTGTAG | CGT |
| 2332915 | CCTCCCTCCCTCCCACCA | ACT |
| 2332916 | GGGACTCTTCCTGAGGCAG | ACG |
| 8012759 | GTCACCTCCTCAGAGAAGC | ACG |
| 10149272 | CTTAGTGCTGTCCCCTGTG | ACT |
| 11158973 | TTATTATGACCCCCTAACACTT | ACT |
| 4903050 | CCTTTGGCTGGTCACCTTC | CGT |
| 10666445 | CAGAAACCACCTGGGTTCCT | ACT |
| 2536143 | GACAGACTCGGTCCCAAGC | CGT |
| 10638231 | CACCCTTGCTATTTTTTTTTTTTTT | ACT |
| 12431636 | GGCTCAATCTCGGCTCACT | ACT |
| 8005904 | CCTCGTGATCCACCCGCC | ACG |
| 11621058 | CTGTAACCAGAGGTGCACAC | ACG |
| 10133224 | CTAAGGTAGGGATTACAGAATA | ACT |
| 12433185 | CAGTTCAGCTGTTTTGCCCA | CGT |
| 740974 | GGACCATTTCCCACACTTGC | ACT |
| 740973 | GTGCATGTCCAGACCCACTT | ACT |
| 740972 | TCGAGCTTGGACTTCCTGG | ACT |
| 740971 | GCCCAGGAAGTCCAAGCTC | ACT |
| 4243642 | AGAGGTATGCTGTGGAATCC | ACT |
| 4415952 | GCTTCTAATATAGTATAAAGCCC | ACG |
| 2215589 | GAGCCTGAAGGGGTGAGAT | ACG |
| 4343211 | GGGACCTCCAGAAGTAAACT | ACT |
| 2536142 | GCTTTTTCCTTGTCCCATCC | ACT |
| 11293340 | TTATAGTCATGATTATGGTTTATTT | CGT |
| 10151620 | ACGACCTGGGATCAGAGCT | ACT |
| 2286840 | CAAAGGCAGCGTCTTCACTT | ACT |
| 8023089 | CCCTTTTCTATGTGGGGCCA | CGT |
| 8008009 | CAAAACTGTTTTCAGCCTCAG | ACG |
| 11419089 | CCCTGTCTCAGAAAAAAAAAAAAA | CGT |
| 10694676 | CCCTGTCTCAGAAAAAAAAAAAAA | CGT |
| 2536141 | GGTTGAGGCTGCATTGAGC | ACG |
| 765247 | ACACAGTGAGCCCATAAAGAA | CGT |
| 7147626 | GGAGCCTGCCACGTAGTC | ACT |
| 10132629 | CCTAGCATGGAGCCTGCCA | ACG |
| 4903051 | CTCTTCTCAAAACAGTGGTAG | ACT |
| 11158974 | GACACCCCACCCCCGTC | ACT |
| 1035021 | GATGTGGGGCCAGCAATGG | CGT |
| 6574093 | GCAGTGGCTCTGCCCCAT | ACT |
| 4899439 | TGCCCTGGCCATTTCCCC | ACT |
| 7155287 | TCTGGGACAGGCCTGGCA | CGT |
| 7154402 | GAGAAGCCAATCTGAAAAGGC | ACT |
| 7154781 | CGGTTCACTCTTGGCGTTG | ACG |
| 6574094 | GGGGTCCCCAGCAGGTG | ACG |
| 10132242 | GCCGTGTGAGCCCACATC | ACG |
| 10146959 | GCATGGCCATGAGGAGGTC | ACT |
| 10147466 | CCCTGTCTCTAAAAGAGAAAGA | ACT |
| 11626411 | AGCTCCTGTCCTGGGGCT | ACT |
| 4899440 | AGAAACAGAGGGAGTCTGAC | ACG |
| 10144553 | GTAAGCCTCCTAAAGGCAGG | ACT |
| 11627663 | TGCTGTGTGGCTGTTGGTG | ACT |
| 11627799 | TTACATTCTGAGACTCACTCC | ACT |
| 11851733 | GAACCTATGGGAGCAAATCAA | ACG |
| 10140317 | TAGAAATACCAGATCGGATACA | CGT |
| 8007375 | AACACACTTCTATTGAGCCCT | ACG |
| 4899441 | TGACAGCTGCTGCCGCTC | ACG |
| 11158975 | GTGTGTGTGTGTGGTTTTTTTT | ACT |
| 11419607 | GTGTGTGTGTGTGGTTTTTTTT | ACT |
| 2109793 | GGCTCACTGCAACCTCCC | ACT |
| 8023156 | GTGGAAAGAAGGGAGCTCATA | ACT |
| 10140579 | CTGTGGCTCTGCCATGATTTT | ACG |
| 7148199 | GAATTTGCTGCAGACAGCTC | ACT |
| 7146916 | CACTGCTGGGTGGGTCTC | ACG |
| 1990439 | GTCACACAGACTTCGTCCC | ACG |
| 6574095 | CAGCTCCGCAGACCTGAG | ACT |
| 10140037 | GGGTGTGGAGGGGCACC | CGT |
| 8007249 | TCAAAAAGGTCATACCAATTCC | ACT |
| 1990438 | ACAGACCTACTACTAGGCCT | ACT |
| 1990437 | CTTCCTTTCCCTTCCCCTC | ACT |
| 10146484 | CTCAGATACATGAGCAGCACA | ACT |
| 11627333 | GGTGAGAGAAATCAGACTGTG | ACT |
| 7155802 | CAGATTAGCTGGCTGGGGT | ACG |
| 2109792 | GCCTGGTTGCAAACTCCTG | ACT |
| 11429363 | ACCTAGCTATTTTTTTTTTTTTTTTT | ACT |
| 8004210 | GGTGTCCTGTGTCCCAGCT | ACT |
| 2098194 | CTGTGTACTCACAGGCTCAA | ACG |
| 2109791 | CGCTAGGTGGTGCCCCAG | ACT |
| 11381729 | TCTCTCTCTTTCTTACTTTTTTTT | ACG |
| 1160094 | AATCTAGGTTGCCTGATTTCC | ACG |
| 11628051 | TCTTTCAAGAGATAATCCATGC | CGT |
| 10136771 | GGGGACTACAAGTGCATGC | ACG |
| 10139903 | AGGTACCTCACTCCTTCCTT | ACT |
| 4903053 | GCCTGGGCAACAAAGCTAGA | ACG |
| 3742836 | GGATCTTCAGGTGGGTGCA | ACT |
| 3742835 | GAAACTACCCTGACTAACCC | ACG |
| 8003233 | GTGCCACCCTGTGTGTGTG | ACT |
| 11621588 | ACCAGCAAGCCAACAAATCC | ACG |
| 740981 | GCATATGTGTGCGTGCATGT | ACT |
| 8019492 | CAGAGCAAGATTCTGTGAAAGA | CGT |
| 8019644 | TTCCTTCTTTCCTTCCTTTCTT | ACG |
| 8019646 | TTCTTTCCTTCTTTCCTTCCTT | ACG |
| 8019653 | AAAGAAAGAAAGAAAGGAAGGAA | ACT |
| 11450794 | AGGAAGGAAAGAAGGAAAGAAA | ACT |
| 11348193 | TCTTTCTTTCTTTCTTTTTTTCTTT | ACG |
| 11450793 | CCTTTCTCTTCTCTTCTCTTCT | ACG |
| 11282735 | CCTTTCTCTTCTCTTCTCTTCT | ACG |
| 2191823 | AGAAGCTGGAAAATATGACTTAT | CGT |
| 12232220 | ACAATGATGGCCGGGCGC | ACT |
| 11158976 | AAAATAAACTAATGCATACTTTGTA | ACG |
| 4903054 | TCCTTCGTAATAAACTGGCATT | ACG |
| 11846755 | CTGCCTCAACTTCATATGCCA | ACT |
| 12431988 | GCTATGCTAGTAGGGGAGG | ACG |
| 12100526 | TCTTGGACATTTTGGCTGTTAT | ACT |
| 11622568 | TCAACCTGTGGGTTTATGGG | ACT |
| 9671230 | AGTCCTGGAGAGGAGGAAG | ACG |
| 12147487 | AGTAGTTTCTGATGGCCTTCT | CGT |
| 4903055 | GCACATGCCACCACACCC | ACT |
| 12147515 | CGGCTCACTGCAACCTCTG | ACG |
| 11625092 | CAGAGATGAGGCAAGATAAGC | ACG |
| 4899442 | GACCTAACTACCAATTCACAG | ACT |
| 2052146 | GAGCTCTGATAGTGATTGTGAGT | ACT |
| 10220553 | CCTGGATATGTTTTTAAAAATGTT | CGT |
| 10220565 | CTGTAATCCCAGCGACTCAG | ACT |
| 2332917 | GGCTTGAATTTACCTGGCATAA | ACT |
| 7158910 | GTCCACTGCCCCCCGAC | ACG |
| 8015749 | AATATCCTCAGGGTTAACCTAT | ACT |
| 8006812 | TCTACCATCATCTCCAGGGA | ACT |
| 8008338 | GACCAGGTGTTGGCAGAGA | ACG |
| 2052145 | ATTCAGATGGGATCACAGAAG | ACT |
| 2052144 | ACTCAGCTCTCAGAGCTGG | ACT |
| 10146692 | GAGGCGCCTCTTCCTCACT | ACT |
| 11627305 | CCGCCCACAAACACACACG | ACT |
| 2079989 | GGAGGAGGCATGTCACATGA | ACG |
| 8018881 | TCAGGTCAGGAGGATGGAG | CGT |
| 4561387 | AGAAGACAAATACTAAACATTGAA | CGT |
| 11622025 | GAGAAAGGAATGGCTATTGATT | ACT |
| 740980 | CACATTGGGTAAATGATGA | ACT |
| 12433250 | TGGAGAAATGAAAATCTCAGGA | ACT |
| 5809590 | GATGTCAGCAGCATTGAGAAT | ACG |
| 758915 | GACATCAGACCTATGCCAGGA | ACT |
| 758914 | CCCCAGTGATCCTGAGAAAT | CGT |
| 7154229 | ACAAAGTCTGCTAACTGGGC | ACG |
| 12437188 | AAGGGATACCTTTGTCCCCA | ACT |
| 7159108 | CAAGGAGCTGCCAAGGACA | CGT |
| 4903058 | CCACCTCACTTTTGTTGCATT | ACT |
| 4899443 | GTAGCCTCCACCTCCCAG | CGT |
| 11848907 | ACTCCAGCCTGGGCGACA | ACT |
| 12432148 | GGAGGTGTTAGTAGGAGAGA | CGT |
| 12435078 | TGCCATCTCCTCTACCTGG | ACG |
| 6574096 | CTCGACACATATCACTCAACA | ACT |
| 11158977 | GCTGGCAGGAGACCCCC | CGT |
| 11629434 | AATCCCATGCCAGCAGGTG | CGT |
| 4378563 | GGCCTCCATGCCCTGCTTA | ACG |
| 2098195 | TAAACCTTTCTATGTTCCTG | ACT |
| 4633653 | CTGGGACAGGGTATGATCC | CGT |
| 7148350 | GTGACCCTTCCACCATCAAC | ACG |
| 740979 | TGCTTTGGGGCAGACTGAC | CGT |
| 740978 | AAGCCTTCCTATCCCCA | ACT |
| 740977 | TGGTTTTCGAATAAGTAGCCAC | ACT |
| 740976 | ATCCAGATGGCCCTGAC | ACT |
| 4903059 | TCCACACCACGCTGCCTC | ACG |
| 4903060 | AGGCCAGAGAAAGAGGCGA | ACG |
| 11852056 | CCCCTCAAGAGCCCCATC | ACT |
| 9635257 | ACCACTGCCGCTGCCCC | ACG |
| 9635258 | CAGGGTTTGTTCTCTGTCTC | ACG |
| 9635259 | CCATCACAGCTTCAAAATCACA | ACT |
| 4899444 | AAATTCCTTAACCTCTGTGAAC | ACG |
| 12435382 | CTCCCCTGGGGCTTCCC | ACG |
| 12435412 | GGGCAGGTCCAATGAAAATCA | ACG |
| 11627381 | AGGACCCCACATTGTGGTAA | ACG |
| 2052143 | TCTACCTCCACTTCCAA | ACG |
| 2052142 | AGCCAGGATAATCTCCTCA | ACG |
| 2052141 | CTTACCCCCAAAGATGTCCA | ACG |
| 8021035 | GAGGCTAGGACCAAAGGAAC | ACT |
| 11158978 | CTCAGGAGGCTGAGGTGG | ACT |
| 11621216 | CATTTACCACTCACAGGGTAT | CGT |
| 10130873 | TTGTCACTTCCTCATGCCTC | ACT |
| 11621358 | CTTGTCACTTCCTCATGCCT | ACG |
| 758913 | CCAGTCTAGTTTTCGATCACC | ACT |
| 11622602 | CAACGTAGGATGTGGTGATC | ACT |
| 10136931 | CCATATTTTCAACATACATACATA | ACG |
| 740975 | ACCAGCTCTCTTTGGAT | ACT |
| 747987 | AACCTGGTTCTGCCATT | ACT |
| 11623819 | GGCAACTCCTTCCACAATGG | ACG |
| 12434445 | CTGAAGCAGCTGGGAGCC | CGT |
| 8003375 | CTGTCAGATGCCACACATCAA | CGT |
| 10143596 | AGATGGATAAATAGATAAATGGAT | ACT |
| 12435321 | TGACTGCAGTGGGCTGCC | ACT |
| 12435401 | TTTTGGGTGTCTGGAATAACG | ACT |
| 8010609 | GGCTGCCTGCCCAGATCC | ACT |
| 11158979 | GACACCAAAACCCAGTGCCT | ACT |
| 11158980 | ATATGCTCACAGTTCAGGCAA | ACG |
| 11627524 | TGCCTGGACAAGGCCCATA | CGT |
| 1126160 | GAAGCCCATCGCTAAGTGTTT | ACT |
| 10136074 | GGTGACACGTTGGGCTTGT | ACT |
| 4903061 | CAAAGGTAGTTGAATCTGATTC | CGT |
| 10665398 | TGTTTGTGTGTGTGTGTGTGT | ACT |
| 6574097 | GTGTGTGTGTGTCTGTGTGT | ACT |
| 9671654 | ACACACACACACAAACACACA | ACT |
| 10140024 | AGGCTGGGCACAGTGGCT | ACG |
| 10142421 | AGATCACCGCAACCTCTGC | ACG |
| 10142593 | GGCAACAAGAGTGAAACTCTA | ACG |
| 11423372 | TCTTGCTCTGTCACCCAGG | ACT |
| 5809592 | TCTTGCTCTGTCACCCAGG | ACT |
| 2332918 | TCTCTCTAATGGGGACC | ACG |
| 2332919 | ACTGGATCCCAGAAGAG | ACT |
| 8007348 | GGAGGCCAAGGAGTTTCAAG | ACT |
| 1990443 | TTCCCCTCAGCTCTTAG | ACG |
| 8007690 | TAGCCATCCTTCCTCAGATG | ACT |
| 3937455 | TCCTTTTTTCCCCACCC | ACT |
| 8012823 | GCTCCTGGAGGGCTTCCAA | ACG |
| 4899445 | CCCTCTCCAGGGCCATGC | ACG |
| 4903062 | CTTGGGCAAACTCCTTAAAATA | ACG |
| 973963 | AACAACCAACTCTCCAG | ACG |
| 10149962 | CAGCAGAACCGAGGTGAAAT | ACT |
| 8003449 | CATTTTCAGGGATGCCTCCT | ACT |
| 11620775 | GCACAAAGCAGCAGGGAAC | ACG |
| 8009089 | ACAGACACTCCCACATCATC | ACT |
| 8008967 | CCGACTTCTCACTCTTTCTG | ACT |
| 12050132 | CAGGCCTCCCCATGCACT | CGT |
| 10133018 | AAATAACCCCAAGAAGCAGAC | ACT |
| 1990442 | CTCCAACTCAGAACATGCTC | ACT |
| 1990441 | CATGAATGTGATTCACATTCTCC | ACT |
| 8015833 | GGAAGAGACGGGCGGAGAA | ACG |
| 1990440 | CGTCAGCAAATGTGTACCGA | ACT |
| 4903063 | CATTGAGATGGGAAGGGCAA | ACT |
| 10139810 | TTTTTCCCATCCCCACCCC | CGT |
| 4903064 | GTGTCAGCAGAAAAAGTGTAC | ACT |
| 2159715 | CATAGGGATAGGAATGGG | ACT |
| 2109795 | TCCCAGAATTTGGAGCC | ACT |
| 2159714 | CAAAAGGATCTGCAAAAG | ACG |
| 11623107 | TAGTGAGCAGGGGAGCCTA | ACT |
| 7152005 | GCTGACCCCAGGCAGGG | ACG |
| 7152489 | CTGGGGCTCGAGCACAGA | ACT |
| 4479167 | GGTGTCAGCAACCACACCC | ACT |
| 4606655 | GCACCTGCCATCATGACCA | ACT |
| 4606656 | CTGCCTTCCCGGTTAAGCA | ACT |
| 4489943 | GTTGCAGTGAGCCGAGATC | ACT |
| 7142642 | TGTTGTTTTAAGCTACTGAGTTT | ACT |
| 8006233 | AGGGTGTGTAATGTAACATTTC | ACT |
| 8007559 | GCCCTGCACTCCCACCCT | CGT |
| 8006156 | TCCCACCCTTGTACACTGC | ACT |
| 1468662 | CTCTGACTGAGGAGAGACC | ACT |
| 11627048 | CCGTGGAGGACCCCTGGA | ACG |
| 6574098 | GACCTCAGGTGATCCACCC | ACT |
| 12433760 | GCCAAGGTGGGCAGATCAC | ACG |
| 12433780 | GAAACCAGCCTGGCCAACA | ACG |
| 12434506 | GGGAAACACAGAAAGACCCTA | CGT |
| 12434673 | ATGGGTGTGAACGGTAGCTT | ACT |
| 12431400 | GCCATTTTAATGGGTGTGAAC | ACT |
| 12431384 | GTGGCTGTGCTTTTGGTTGC | ACG |
| 4903067 | GCTGCTAATGTACACAACAATG | ACG |
| 2332920 | GAGATGGGGTTTTCCCATGT | ACG |
| 2215591 | TTCAATAAAATGTACTCATTCAAA | ACT |
| 12433064 | GACCAGGAGTTCAAGACCAG | CGT |
| 12436263 | GACCAGCCTGGACAACATGA | ACT |
| 12433097 | TCACTGCAACCTCTGCGTC | ACG |
| 12433184 | GTGTGCATGCCACCACGC | CGT |
| 8015900 | TGTGACCTCAGCTCACTGC | CGT |
| 11296981 | AGATGTGTAGATGACTTACACT | CGT |
| 11344025 | AGATGTGTAGATGACTTACACT | CGT |
| 11464204 | CGAGAGCTCATCTCCACTAAA | CGT |
| 11441172 | GTGCCTGGCTAATTAAAAAAAAAA | CGT |
| 11381807 | CTCATCTCCACTAAATTTTTTTTTT | CGT |
| 10873251 | CCACCTTGGCCTCCCAAAG | ACG |
| 2109794 | AGCACTTTGGGAGGCCAAG | ACT |
| 10873252 | GCATGTGCTACCACGCCC | ACT |
| 4520784 | CCAGGTTCAAGTGATCCTCC | ACG |
| 2877820 | TGCCCTGTTCTGCACCTTTAAAA | CGT |
| 2877821 | CCCTGTTCTGCACCTTTAAA | CGT |
| 4569195 | AGGGAGAAAAAAATAAAAGGACA | ACT |
| 11628905 | GGGAGTGGGAGGAAGCCAA | ACT |
| 2191822 | AGGAAAGGAATGTCTGCCCC | ACT |
| 2191821 | ATGTGGGTTTGGACTGGGGCT | ACT |
| 11464689 | CGTTTTCTTTTTCTTTTTTTTTTTTT | ACT |
| 12431959 | GCTGAAGTAGGCGGATCAC | ACG |
| 4903068 | CTCTCTCACAGAGCACACTC | ACT |
| 1544579 | GACATGCATCAAAGCAGCTG | ACT |
| 7155879 | CCTTTTCGCTGAAAAGAGGG | CGT |
| 12433603 | GGAAGAAGCCCTCTTTTCAG | ACT |
| 11351432 | ACTCCATCTCAAAAAAAAAAAAAAA | ACT |
| 11448438 | CAAACCACTGTATAAGTCCAG | ACT |
| 4243643 | CTCAGCCTCCTGAGATGCT | ACG |
| 4140952 | CATGCTACAAGCAACCTTGG | ACT |
| 8005372 | AACAGTGGTTGAGTACCTGC | ACG |
| 8004109 | CATGACCCTGTACCCAGCAT | ACT |
| 8008398 | TCTATGAACCTGTTGAAGGCA | ACT |
| 8009692 | CTTCTCCTCCCCATAGCTCT | ACT |
| 2332921 | TGTCTCCAGGGTCTGGGG | ACT |
| 12050320 | CAGCAATCTATGCACAGAGAA | ACT |
| 12050323 | CAAAGGCTGAGAAAGACTGAA | ACT |
| 8010316 | AAATTTAACCATCATCTGCCAG | ACT |
| 7154293 | GGTCAACATGGTGAAACCCC | ACT |
| 12147969 | AAAATAAGCCTGGCGCACCT | ACG |
| 2215590 | CAGGGCCAGCCATGAACGT | ACG |
| 4265749 | GGATCATCCAAGTATGCTCC | ACG |
| 1004552 | TCTTGGCTCAGTGCTGC | ACG |
| 4307892 | GAGCAATGAACACCAATATCC | ACG |
| 12431973 | CTGAAGCAGGAGGATCGCTT | ACT |
| 12431975 | CGCTTGAGCCCAGGAGTTC | ACT |
| 767874 | CATTTGCCCTATCTGTAGTTC | ACT |
| 11628597 | CATCTCCCCGAGGCACCC | ACT |
| 10129371 | TCCAGTGATATGCTTCCCTC | ACT |
| 4903069 | GAACCTGGGAGGTGGAGGT | ACT |
| 4903070 | TGTTGGCCAGACTGGTCTC | ACT |
| 4903071 | GGGATCAGTAAACCTTTCCC | CGT |
| 11622224 | CTTCATGAAGGCCGCAGAC | ACT |
| 10459455 | CTCGACTCATGGCAACCTC | ACG |
| 9323577 | CTGGGCAACAGAGTGAGAC | ACG |
| 10624648 | CTTCCCTGACCCCCAAACAA | ACT |
| 3058953 | CTTCCCTGACCCCCAAACAA | ACT |
| 11158981 | ACCTCAAAACCTGACTATAGAA | ACT |
| 11158982 | CTCATCAAAAGTAAAATGGGAAAA | ACT |
| 11158983 | TTTTTGTTCATTGGCTAATTTCTT | CGT |
| 1860750 | CCTTAATCATGTTTATTTATTTATTT | CGT |
| 1860749 | TTAAGGATAAAGCAATCCAG | ACG |
| 1860748 | TCTTGGAGCCATATTTTATTTG | ACT |
| 1860747 | GACAGAGGAGAGCTCCCC | ACT |
| 11419679 | AGCCCAGTAGTAAGGAAAGAA | CGT |
| 8011777 | CAGAGTGAGACTCCGTCTCA | ACT |
| 6574099 | ATGTTAGCCAGGATGGTCTC | ACT |
| 8010957 | GATTATGAGCCACTGCACCC | ACT |
| 11626895 | GCCCATCTTCTTGGGTGTGT | ACG |
| 10151431 | CTGCCTCCTGGGTTCAAGC | ACT |
| 4903072 | GTGTGACAAGCCAATATTCCC | CGT |
| 4589489 | CCACACCATTACCAGGCCA | ACT |
| 9671327 | CCACTGTGCCCAGCCTAAA | CGT |
| 4899446 | GCTGGGCACAGTGGCTCA | ACG |
| 10144501 | AGATCATACCACTGCACTCC | CGT |
| 5809594 | GTGCGCACACACACACACA | ACG |
| 720317 | CATGTGTGTGTGTGTGCGC | ACT |
| 8014316 | CCCGGCATTCTCTAGCACC | ACG |
| 11158984 | GGGACGTTCAAAGGGGAGC | ACT |
| 6420906 | GTACAATGTACAAAATGTCCACT | ACG |
| 8004055 | CCCACAGACTCCTACGGG | CGT |
| 12434800 | TGTTGACTGCCTTACACACC | ACT |
| 4243644 | TTACCCTACAGCTGATTTTTTTT | CGT |
| 4899447 | CCTTGCGTGTGTGTGTGTGT | ACT |
| 4903073 | TATGCCTCAGCAAGCATGCA | ACG |
| 11848634 | TGCTGATTGTAAACAATGCACA | ACT |
| 4899448 | CTGCTCTTGACTAGTCTTAAAAA | ACT |
| 10131564 | CTCTGTCACTGACTGGCCC | ACT |
| 10134150 | AGAAGAAGCATTGGTCTGTTTA | ACT |
| 763388 | CACTCATGCCTCAAGCCAAT | ACT |
| 11845938 | TGAGGCATGAGTGATCAGCA | ACT |
| 11845946 | ACCTAAGTTTCCTGACATTCC | CGT |
| 11845915 | TACAATTCACCCAGCTCCTC | ACT |
| 10137463 | CCATCTGCCTGTCCTCCTG | ACT |
| 6574100 | CAAGAGCTGGAGTGATTTGTTT | CGT |
| 7140344 | ACATGCTAGGAAGTAGGCAAT | ACT |
| 10646889 | CACCTGGGTTTTTTTTTTTTTTTTT | ACT |
| 7146287 | TTGAACATGCAAAGTATTTCTGA | ACG |
| 10605948 | GCTTCCTCCCCCCCCCC | CGT |
| 5809595 | CACCATTTCTGTTTTTTTTTTTTTT | ACT |
| 1035099 | TTGGTCAACATCGCAGC | CGT |
| 7159877 | ACAAACCTCCACCTCCTGG | ACT |
| 7142579 | CATGTCACAGTTCTGAATAGAG | ACG |
| 12431762 | GAGCTAGGGGTCTGAATTAG | ACG |
| 12431764 | ACTTAAGGAGAGCTAGGGGT | ACG |
| 10647999 | CTTTCTTTCTTTCTCTTTCTTTCT | ACG |
| 6574101 | GGAGCTCAGTGGCACACGA | ACG |
| 6574102 | GAGCAAGTGGAAGATGAGAC | ACT |
| 1861162 | CGATGTGTAGAAAAGGGCAC | ACT |
| 12432972 | GGAGGCCGACACTGACCA | ACT |
| 10146134 | TTATACTTTTGATGGCTCCTAC | CGT |
| 10146616 | TCCTGACCATTCTACTGCCT | ACT |
| 10146769 | GGTCAGGAGGAGAGAGGGA | ACT |
| 8018936 | GGCAAGGACATAAAGCTTCAT | ACG |
| 10483849 | GCCTGGAGATTTCGTGGTC | ACG |
| 2160137 | GGGTACCTGGGAGTCAGAC | ACT |
| 2192595 | CTTGGGCATCCTAGTAGAGA | CGT |
| 2192594 | CGGATTTTCATGTCCTGGAAA | ACT |
| 11271695 | CTCCTGCCATGATTCTGAGG | CGT |
| 10747301 | GATTTCAGACCTACCTGGGC | ACT |
| 12050363 | GTACAGCCTCCCTCCTGC | ACT |
| 12050368 | GCCTGGAAAAGCTGCAGAGA | ACG |
| 12432949 | ACCCCATCAACAAGTGGGC | ACT |
| 7401375 | ACCATCTCACACCAGTTAGAA | CGT |
| 8019682 | ATGTTTTAGCGAAGAGACTGG | ACG |
| 8021248 | ATTTTCTCTTGCTGCTGCCAT | ACT |
| 11158985 | GTGAGGACACAGCCAAACC | CGT |
| 10483850 | GGAAGGAAGTGGCCAGCAC | ACT |
| 8010875 | CTCATTTTTCCCTTCTGTTCAC | ACT |
| 11626844 | CCTGAGAGAAGCTGCTTCAC | ACT |
| 982972 | AAGCACTGAAGGGGCACAAA | ACT |
| 7161198 | ATAGAGATACCAGCGTATCATT | ACT |
| 7160347 | TTAGCTTTATTTATACTGGAGGA | ACG |
| 8007139 | GATTCCATCTCCAAAAAAAAAAAA | ACT |
| 8014483 | ACTCTCCAAGGCAACTCACT | ACG |
| 12437072 | CCAACGTCACAGTCCTAAGT | ACT |
| 8004767 | GCACCGCGTTGCCGCCC | ACT |
| 12588830 | TGCCCTGCCAAAAAACAAGG | ACT |
| 7158449 | CCCAGGCTGGAGTGCAGTA | ACT |
| 6574103 | GGCTGGCAACCAAGACATG | ACG |
| 8020478 | GGTGGCTTACGCCTGTAATC | ACG |
| 4903077 | ATTACAGGCATGAGCCTAGC | ACG |
| 8012359 | CGGAGAGAAACTTTGTATGCA | ACG |
| 4899449 | AATGGCTAAAATGGTACACTGT | ACT |
| 10690289 | GTGATCTGGGACACAGAACTT | ACG |
| 6574104 | CTGGCACATGTCTAATCCCC | CGT |
| 2110552 | TCCGGTTACTGGTGTTTCAC | ACT |
| 8008256 | GGGGAGAGATGAGGTGTTG | ACG |
| 759283 | CAGGAATGTGACCCCCACC | CGT |
| 4999177 | AACACCCTGTCTCAAAAAATAAT | ACT |
| 2333006 | CATTATGTTTCCCAGGTATGCA | ACT |
| 8006178 | TCTCTAAGCTTGTGTTCCCC | ACT |
| 11158986 | TACCGAACCCACAACTGATTT | ACT |
| 7150625 | GGCGGCTTAACAGTTCCAC | ACG |
| 8007768 | TACACGAGCAGACACACGC | ACT |
| 8007058 | GAGCGCCCTGCAAACTCC | ACT |
| DPF3-AA | CCCGTCGCTTCCCCTGAG | CGT |
| 2215901 | CTCTGGGCTTTGACAGCTC | ACG |
| 3814866 | GAAAAGGAGCTACCAGCGC | ACT |
| 3814864 | TGGAAGGCTGCCGTTTCGT | ACT |

### Genetic Analysis of Allelotyping Results

Allelotyping results are shown for cases and controls in Table 44. The allele frequency for the A2 allele is noted in the fifth and sixth columns for breast cancer pools and control pools, respectively, where “AF” is allele frequency. SNPs with blank allele frequencies were untyped.

TABLE 44

| | | | | | | | | Breast Cancer |
|---|---|---|---|---|---|---|---|---|
| | Position in | Chromosome | A1/A2 | | | | | Associated |
| dbSNP rs# | SEQ ID NO: 6 | Position | Allele | Case AF | Control AF | p-Value | OR | Allele |
| 11846274 | 207 | 71066357 | G/A | G = 0.001 | G = 0.002 | 0.897 | 3.28 | A |
| | | | | A = 0.999 | A = 0.998 | | | |
| 2526935 | 486 | 71066636 | T/G | T = 0.749 | T = 0.699 | 0.134 | 0.78 | T |
| | | | | G = 0.251 | G = 0.301 | | | |
| 929328 | 1745 | 71067895 | T/C | T = 0.559 | T = 0.53 | 0.372 | 0.89 | T |
| | | | | C = 0.441 | C = 0.470 | | | |
| 3742840 | 1922 | 71068072 | A/G | A = 0.999 | A = 0.998 | 0.935 | 0.68 | A |
| | | | | G = 0.001 | G = 0.002 | | | |
| 9671739 | 2190 | 71068340 | A/C | A = 0.001 | A = 0.001 | 0.963 | 0.49 | A |
| | | | | C = 0.999 | C = 0.999 | | | |
| 8006208 | 2590 | 71068740 | C/T | C = 1.000 | C = 1.000 | 0.991 | 0.31 | C |
| | | | | T = 0.000 | T = 0.000 | | | |
| 8006230 | 2637 | 71068787 | T/C | T = 0.002 | T = 0.003 | 0.924 | 1.99 | C |
| | | | | C = 0.998 | C = 0.997 | | | |
| 8007836 | 2804 | 71068954 | C/T | C = 0.001 | C = 0.001 | 0.976 | 0.60 | C |
| | | | | T = 0.999 | T = 0.999 | | | |
| 8006889 | 2806 | 71068956 | G/A | G = 1.000 | G = 1.000 | 1 | | |
| | | | | A = 0 | A = 0 | | | |
| 8010823 | 2895 | 71069045 | C/T | C = 1.000 | C = 0.999 | 0.911 | 0.26 | C |
| | | | | T = 0.000 | T = 0.001 | | | |
| 8011029 | 3109 | 71069259 | A/T | A = 0.997 | A = 0.999 | 0.858 | 1.89 | T |
| | | | | T = 0.003 | T = 0.001 | | | |
| 10649920 | 3185 | 71069335 | —/AA | — = 0.579 | — = 0.575 | 0.881 | 0.98 | — |
| | | | | AA = 0.421 | AA = 0.425 | | | |
| 2803956 | 3355 | 71069505 | A/G | A = 0.621 | A = 0.571 | 0.118 | 0.81 | A |
| | | | | G = 0.379 | G = 0.429 | | | |
| 2803957 | 3642 | 71069792 | C/T | C = 0.732 | C = 0.758 | 0.382 | 1.15 | T |
| | | | | T = 0.268 | T = 0.242 | | | |
| 2526934 | 3805 | 71069955 | T/C | T = 1.000 | T = 1.000 | 0.98 | | C |
| | | | | C = 0.000 | C = 0 | | | |
| 9671558 | 4236 | 71070386 | A/G | A = 0.001 | A = 0.001 | 0.951 | 0.36 | A |
| | | | | G = 0.999 | G = 0.999 | | | |
| 2526933 | 4331 | 71070481 | C/G | C = 0.699 | C = 0.705 | 0.85 | 1.03 | G |
| | | | | G = 0.301 | G = 0.295 | | | |
| 2803959 | 4509 | 71070659 | T/C | T = 0.565 | T = 0.558 | 0.829 | 0.97 | T |
| | | | | C = 0.435 | C = 0.442 | | | |
| 2526932 | 4959 | 71071109 | C/T | C = 0.559 | C = 0.6 | 0.226 | 1.18 | T |
| | | | | T = 0.441 | T = 0.400 | | | |
| 2803960 | 5009 | 71071159 | G/A | G = 0.002 | G = 0.002 | 0.952 | 1.47 | A |
| | | | | A = 0.998 | A = 0.998 | | | |
| 12434675 | 5676 | 71071826 | G/A | G = 0.71 | G = 0.682 | 0.344 | 0.88 | G |
| | | | | A = 0.290 | A = 0.318 | | | |
| 4899435 | 6507 | 71072657 | G/C | G = 0.002 | G = 0.003 | 0.932 | 1.52 | C |
| | | | | C = 0.998 | C = 0.997 | | | |
| 2526931 | 6695 | 71072845 | C/T | C = 0 | C = 0.001 | 0.906 | | T |
| | | | | T = 1.000 | T = 0.999 | | | |
| 2803961 | 6717 | 71072867 | T/C | T = 0.509 | T = 0.494 | 0.647 | 0.94 | T |
| | | | | C = 0.491 | C = 0.506 | | | |
| 2803962 | 7131 | 71073281 | C/T | C = 0.443 | C = 0.426 | 0.598 | 0.93 | C |
| | | | | T = 0.557 | T = 0.574 | | | |
| 12590634 | 7873 | 71074023 | C/T | C = 0.639 | C = 0.612 | 0.393 | 0.89 | C |
| | | | | T = 0.361 | T = 0.388 | | | |
| 11327712 | 7922 | 71074072 | —/C | — = 0 | — = 0 | 1 | | |
| | | | | C = 1.000 | C = 1.000 | | | |
| 2803963 | 8652 | 71074802 | A/G | A = 0.525 | A = 0.577 | 0.109 | 1.23 | G |
| | | | | G = 0.475 | G = 0.423 | | | |
| 11844834 | 9817 | 71075967 | G/A | G = 0.998 | G = 0.988 | 0.309 | 0.23 | G |
| | | | | A = 0.002 | A = 0.012 | | | |
| 11320415 | 10272 | 71076422 | —/A | — = 0 | — = 0 | 1 | | |
| | | | | A = 1.000 | A = 1.000 | | | |
| 8012752 | 10823 | 71076973 | T/C | T = 0.002 | T = 0.002 | 0.925 | 1.65 | C |
| | | | | C = 0.998 | C = 0.998 | | | |
| 11623878 | 11465 | 71077615 | T/C | T = 0.127 | T = 0.104 | 0.264 | 0.79 | T |
| | | | | C = 0.873 | C = 0.896 | | | |
| 11623926 | 11639 | 71077789 | C/T | C = 0.975 | C = 0.98 | 0.716 | 1.21 | T |
| | | | | T = 0.025 | T = 0.020 | | | |
| 12434442 | 12177 | 71078327 | T/C | T = 0.354 | T = 0.412 | 0.0584 | 1.28 | C |
| | | | | C = 0.646 | C = 0.588 | | | |
| 2526930 | 12604 | 71078754 | G/A | G = 0.929 | G = 0.911 | 0.32 | 0.78 | G |
| | | | | A = 0.071 | A = 0.089 | | | |
| 12435306 | 13363 | 71079513 | G/A | G = 0.425 | G = 0.455 | 0.354 | 1.13 | A |
| | | | | A = 0.575 | A = 0.545 | | | |
| 10144028 | 13454 | 71079604 | C/G | C = 1.000 | C = 1.000 | 0.97 | 2.29 | G |
| | | | | G = 0.000 | G = 0.000 | | | |
| 4899436 | 13540 | 71079690 | C/T | C = 0.999 | C = 0.999 | 0.966 | 0.75 | C |
| | | | | T = 0.001 | T = 0.001 | | | |
| 7140593 | 13923 | 71080073 | A/C | A = 1.000 | A = 0.999 | 0.981 | 0.76 | A |
| | | | | C = 0.000 | C = 0.001 | | | |
| 2107687 | 14507 | 71080657 | T/C | T = 0.999 | T = 1.000 | 0.926 | 3.80 | C |
| | | | | C = 0.001 | C = 0.000 | | | |
| 991882 | 16550 | 71082700 | C/A | C = 0.001 | C = 0.002 | 0.875 | 3.36 | A |
| | | | | A = 0.999 | A = 0.998 | | | |
| 3327 | 17641 | 71083791 | C/T | C = 0.003 | C = 0.154 | 2.36E−07 | 60.70 | T |
| | | | | T = 0.997 | T = 0.846 | | | |
| 2526928 | 18903 | 71085053 | T/C | T = 1.000 | T = 1.000 | 0.994 | 0.90 | T |
| | | | | C = 0.000 | C = 0.000 | | | |
| 7401336 | 19395 | 71085545 | A/G | A = 1.000 | A = 1.000 | 0.955 | 2.83 | G |
| | | | | G = 0.000 | G = 0.000 | | | |
| 2526926 | 195277 | 1085677 | T/A | T = 0.953 | T = 0.9 | 0.00403 | 0.45 | T |
| | | | | A = 0.047 | A = 0.100 | | | |
| 994760 | 20369 | 71086519 | T/C | T = 1.000 | T = 1.000 | 0.949 | 0.36 | T |
| | | | | C = 0.000 | C = 0.000 | | | |
| 994759 | 20505 | 71086655 | G/T | G = 0.469 | G = 0.511 | 0.181 | 1.18 | T |
| | | | | T = 0.531 | T = 0.489 | | | |
| 994758 | 20562 | 71086712 | C/T | C = 0.916 | C = 0.884 | 0.118 | 0.70 | C |
| | | | | T = 0.084 | T = 0.116 | | | |
| 11848404 | 20907 | 71087057 | A/T | A = 0.002 | A = 0.008 | 0.553 | 4.19 | T |
| | | | | T = 0.998 | T = 0.992 | | | |
| 2526925 | 20949 | 71087099 | C/T | C = 0.999 | C = 0.998 | 0.881 | 0.46 | C |
| | | | | T = 0.001 | T = 0.002 | | | |
| 11844236 | 21278 | 71087428 | T/G | T = 0.008 | T = 0.036 | 0.0996 | 4.84 | G |
| | | | | G = 0.992 | G = 0.964 | | | |
| 2526924 | 21314 | 71087464 | C/T | C = 0.005 | C = 0.002 | 0.818 | 0.36 | C |
| | | | | T = 0.995 | T = 0.998 | | | |
| 2803965 | 21905 | 71088055 | C/G | C = 0.767 | C = 0.781 | 0.619 | 1.08 | G |
| | | | | G = 0.233 | G = 0.219 | | | |
| 2526923 | 22252 | 71088402 | G/C | G = 0.179 | G = 0.189 | 0.75 | 1.07 | C |
| | | | | C = 0.821 | C = 0.811 | | | |
| 7152467 | 22941 | 71089091 | C/T | C = 0.999 | C = 0.998 | 0.941 | 0.73 | C |
| | | | | T = 0.001 | T = 0.002 | | | |
| 11623462 | 23542 | 71089692 | C/G | C = 0.205 | C = 0.168 | 0.173 | 0.78 | C |
| | | | | G = 0.795 | G = 0.832 | | | |
| 2803966 | 24677 | 71090827 | C/T | C = 0.44 | C = 0.42 | 0.522 | 0.92 | C |
| | | | | T = 0.560 | T = 0.580 | | | |
| 2526922 | 25009 | 71091159 | T/C | T = 0.543 | T = 0.534 | 0.777 | 0.96 | T |
| | | | | C = 0.457 | C = 0.466 | | | |
| 2526920 | 25618 | 71091768 | C/T | C = 0.604 | C = 0.616 | 0.715 | 1.05 | T |
| | | | | T = 0.396 | T = 0.384 | | | |
| 11848135 | 26082 | 71092232 | T/C | T = 1.000 | T = 1.000 | 1 | | |
| | | | | C = 0 | C = 0 | | | |
| 11629210 | 26136 | 71092286 | C/A | C = 1.000 | C = 1.000 | 0.984 | 0.59 | C |
| | | | | A = 0.000 | A = 0.000 | | | |
| 2803967 | 26491 | 71092641 | C/T | C = 0.001 | C = 0.001 | 0.989 | 0.88 | C |
| | | | | T = 0.999 | T = 0.999 | | | |
| 16455 | 28873 | 71095023 | —/GCC | — = 0.725 | — = 0.745 | 0.498 | 1.11 | GCC |
| | | | | GCC = 0.275 | GCC = 0.255 | | | |
| 757655 | 28886 | 71095036 | G/A | G = 0.613 | G = 0.665 | 0.106 | 1.25 | A |
| | | | | A = 0.387 | A = 0.335 | | | |
| 5809581 | 29105 | 71095255 | —/ | — = 0.005 | — = 0.002 | 0.761 | 0.39 | — |
| | | | TCTGTA | TCTGTAAT = | TCTGTAAT = | | | |
| | | | AT | 0.995 | 0.998 | | | |
| 3057139 | 29106 | 71095256 | —/ | — = 0.005 | — = 0.004 | 0.854 | 0.67 | — |
| | | | CTGTA | CTGTAATT = | CTGTAATT = | | | |
| | | | ATT | 0.995 | 0.996 | | | |
| 2247658 | 29572 | 71095722 | C/T | C = 0.801 | C = 0.797 | 0.882 | 0.98 | C |
| | | | | T = 0.199 | T = 0.203 | | | |
| 11625609 | 30640 | 71096790 | C/T | C = 0.999 | C = 1.000 | 0.891 | 3.18 | T |
| | | | | T = 0.001 | T = 0.000 | | | |
| 8005580 | 30801 | 71096951 | A/G | A = 0.998 | A = 0.998 | 0.966 | 0.84 | A |
| | | | | G = 0.002 | G = 0.002 | | | |
| 8010472 | 30962 | 71097112 | G/A | G = 1.000 | G = 1.000 | 0.917 | 0.18 | G |
| | | | | A = 0.000 | A = 0.000 | | | |
| 2526919 | 31662 | 71097812 | T/C | T = 0.557 | T = 0.539 | 0.582 | 0.93 | T |
| | | | | C = 0.443 | C = 0.461 | | | |
| 2803968 | 34374 | 71100524 | G/A | G = 0.359 | G = 0.308 | 0.0874 | 0.79 | G |
| | | | | A = 0.641 | A = 0.692 | | | |
| 11623943 | 34494 | 71100644 | T/C | T = 0.001 | T = 0.002 | 0.903 | 2.17 | C |
| | | | | C = 0.999 | C = 0.998 | | | |
| 11623946 | 34501 | 71100651 | G/C | G = 0.002 | G = 0.002 | 0.967 | 0.77 | G |
| | | | | C = 0.998 | C = 0.998 | | | |
| 1557926 | 34544 | 71100694 | C/G | C = 0.646 | C = 0.658 | 0.692 | 1.05 | G |
| | | | | G = 0.354 | G = 0.342 | | | |
| 12434611 | 35493 | 71101643 | A/G | A = 0.486 | A = 0.528 | 0.193 | 1.18 | G |
| | | | | G = 0.514 | G = 0.472 | | | |
| 2526918 | 35594 | 71101744 | T/G | T = 0.998 | T = 0.998 | 0.992 | 1.04 | G |
| | | | | G = 0.002 | G = 0.002 | | | |
| 5809582 | 35901 | 71102051 | —/T | — = 0.93 | — = 0.925 | 0.784 | 0.93 | — |
| | | | | T = 0.070 | T = 0.075 | | | |
| 8008279 | 35940 | 71102090 | C/G | C = 0.835 | C = 0.852 | 0.505 | 1.14 | G |
| | | | | G = 0.165 | G = 0.148 | | | |
| 2803969 | 36064 | 71102214 | T/C | T = 0.993 | T = 0.991 | 0.863 | 0.76 | T |
| | | | | C = 0.007 | C = 0.009 | | | |
| 2526917 | 36105 | 71102255 | T/G | T = 1.000 | T = 1.000 | 0.951 | 3.23 | G |
| | | | | G = 0.000 | G = 0.000 | | | |
| 8008774 | 36160 | 71102310 | T/C | T = 0.375 | T = 0.338 | 0.219 | 0.85 | T |
| | | | | C = 0.625 | C = 0.662 | | | |
| 2803970 | 36161 | 71102311 | T/G | T = 1.000 | T = 1.000 | 0.991 | 0.82 | T |
| | | | | G = 0.000 | G = 0.000 | | | |
| 8010255 | 36308 | 71102458 | T/C | T = 0.778 | T = 0.812 | 0.2 | 1.23 | C |
| | | | | C = 0.222 | C = 0.188 | | | |
| 2997642 | 36335 | 71102485 | A/G | A = 1.000 | A = 1.000 | 0.971 | 2.42 | G |
| | | | | G = 0.000 | G = 0.000 | | | |
| 2803971 | 36365 | 71102515 | G/A | G = 0.352 | G = 0.315 | 0.223 | 0.85 | G |
| | | | | A = 0.648 | A = 0.685 | | | |
| 11313760 | 36488 | 71102638 | —/G | — = 0.369 | — = 0.327 | 0.163 | 0.83 | — |
| | | | | G = 0.631 | G = 0.673 | | | |
| 2803972 | 36576 | 71102726 | G/T | G = 0.003 | G = 0.005 | 0.814 | 1.84 | T |
| | | | | T = 0.997 | T = 0.995 | | | |
| 4903038 | 36641 | 71102791 | A/G | A = 0.001 | A = 0.002 | 0.887 | 4.87 | G |
| | | | | G = 0.999 | G = 0.998 | | | |
| 2526916 | 36933 | 71103083 | A/T | A = 0.998 | A = | | | |
| | | | | T = 0.002 | T = | | | |
| 4903040 | 36955 | 71103105 | C/T | C = 1.000 | C = 1.000 | 0.953 | 0.51 | C |
| | | | | T = 0.000 | T = 0.000 | | | |
| 2803974 | 36967 | 71103117 | T/A | T = 0.986 | T = 0.998 | 0.457 | 5.99 | A |
| | | | | A = 0.014 | A = 0.002 | | | |
| 2803975 | 37243 | 71103393 | C/T | C = 0.264 | C = 0.235 | 0.365 | 0.86 | C |
| | | | | T = 0.736 | T = 0.765 | | | |
| 2803976 | 38329 | 71104479 | C/T | C = 0.003 | C = 0.002 | 0.865 | 0.43 | C |
| | | | | T = 0.997 | T = 0.998 | | | |
| 2803977 | 38367 | 71104517 | T/G | T = 0.007 | T = 0.002 | 0.718 | 0.31 | T |
| | | | | G = 0.993 | G = 0.998 | | | |
| 10139909 | 38476 | 71104626 | C/T | C = 0.001 | C = 0.001 | 0.994 | 0.94 | C |
| | | | | T = 0.999 | T = 0.999 | | | |
| 722945 | 38510 | 71104660 | G/A | G = 0.751 | G = 0.781 | 0.283 | 1.18 | A |
| | | | | A = 0.249 | A = 0.219 | | | |
| 917069 | 39396 | 71105546 | A/G | A = 0.809 | A = 0.819 | 0.693 | 1.07 | G |
| | | | | G = 0.191 | G = 0.181 | | | |
| 2803978 | 39703 | 71105853 | C/A | C = 0.004 | C = 0.002 | 0.869 | 0.56 | C |
| | | | | A = 0.996 | A = 0.998 | | | |
| 2332890 | 39801 | 71105951 | A/G | A = 0.001 | A = 0.002 | 0.96 | 2.01 | G |
| | | | | G = 0.999 | G = 0.998 | | | |
| 1989638 | 40078 | 71106228 | T/C | T = 0.333 | T = 0.303 | 0.309 | 0.87 | T |
| | | | | C = 0.667 | C = 0.697 | | | |
| 10679174 | 40260 | 71106410 | GGG/— | GGG = 0.195 | GGG = 0.173 | 0.373 | 0.86 | GGG |
| | | | | — = 0.805 | — = 0.827 | | | |
| 2526915 | 40513 | 71106663 | C/T | C = 0.364 | C = 0.343 | 0.498 | 0.91 | C |
| | | | | T = 0.636 | T = 0.657 | | | |
| 2526914 | 40889 | 71107039 | T/C | T = 0.106 | T = 0.038 | 0.000287 | 0.33 | T |
| | | | | C = 0.894 | C = 0.962 | | | |
| 11158964 | 41091 | 71107241 | G/A | G = 1.000 | G = 1.000 | 1 | | |
| | | | | A = 0 | A = 0 | | | |
| 11158965 | 41872 | 71108022 | A/G | A = 0.999 | A = 0.999 | 0.962 | 1.26 | G |
| | | | | G = 0.001 | G = 0.001 | | | |
| 740081 | 42112 | 71108262 | A/G | A = 0.468 | A = 0.441 | 0.405 | 0.90 | A |
| | | | | G = 0.532 | G = 0.559 | | | |
| 740080 | 42292 | 71108442 | T/G | T = 1.000 | T = 1.000 | 0.976 | | G |
| | | | | G = 0.000 | G = 0 | | | |
| 10140582 | 42373 | 71108523 | T/C | T = 0.001 | T = 0 | 0.931 | 0.00 | T |
| | | | | C = 0.999 | C = 1.000 | | | |
| 2526913 | 44877 | 71111027 | A/G | A = 0.192 | A = 0.128 | 0.0186 | 0.62 | A |
| | | | | G = 0.808 | G = 0.872 | | | |
| 4899437 | 45124 | 71111274 | C/G | C = 0.649 | C = 0.621 | 0.376 | 0.89 | C |
| | | | | G = 0.351 | G = 0.379 | | | |
| 10145678 | 45831 | 71111981 | T/C | T = 0.998 | T = 0.996 | 0.738 | 0.42 | T |
| | | | | C = 0.002 | C = 0.004 | | | |
| 2107686 | 46030 | 71112180 | T/C | T = 0.001 | T = 0.002 | 0.957 | 1.97 | C |
| | | | | C = 0.999 | C = 0.998 | | | |
| 2107685 | 46270 | 71112420 | T/C | T = 0.985 | T = 0.998 | 0.338 | 7.09 | C |
| | | | | C = 0.015 | C = 0.002 | | | |
| 2107684 | 46309 | 71112459 | G/A | G = 0.678 | G = 0.637 | 0.176 | 0.84 | G |
| | | | | A = 0.322 | A = 0.363 | | | |
| 7154188 | 46545 | 71112695 | C/G | C = 1.000 | C = 0.994 | 0.8 | 0.00 | C |
| | | | | G = 0 | G = 0.006 | | | |
| 4903041 | 47162 | 71113312 | C/T | C = 0.655 | C = 0.602 | 0.099 | 0.80 | C |
| | | | | T = 0.345 | T = 0.398 | | | |
| 7159637 | 47267 | 71113417 | T/C | T = 0.004 | T = 0.005 | 0.875 | 1.54 | C |
| | | | | C = 0.996 | C = 0.995 | | | |
| 6574089 | 49077 | 71115227 | A/G | A = 1.000 | A = 0.999 | 0.912 | 0.15 | A |
| | | | | G = 0.000 | G = 0.001 | | | |
| 12589753 | 49377 | 71115527 | A/G | A = 0 | A = 0.001 | 0.935 | | G |
| | | | | G = 1.000 | G = 0.999 | | | |
| 4899438 | 50126 | 71116276 | G/C | G = 0.001 | G = 0.001 | 0.983 | 0.73 | G |
| | | | | C = 0.999 | C = 0.999 | | | |
| 4903042 | 50385 | 71116535 | G/T | G = 1.000 | G = 1.000 | 0.936 | 6.18 | T |
| | | | | T = 0.000 | T = 0.000 | | | |
| 4903043 | 50403 | 71116553 | A/T | A = 0.001 | A = 0 | 0.977 | 0.00 | A |
| | | | | T = 0.999 | T = 1.000 | | | |
| 2803980 | 50422 | 71116572 | G/A | G = 0.478 | G = 0.511 | 0.295 | 1.14 | A |
| | | | | A = 0.522 | A = 0.489 | | | |
| 10483848 | 50893 | 71117043 | T/A | T = 0.001 | T = 0.001 | 0.996 | 0.95 | T |
| | | | | A = 0.999 | A = 0.999 | | | |
| 2240344 | 52031 | 71118181 | G/C | G = 0.664 | G = 0.636 | 0.364 | 0.88 | G |
| | | | | C = 0.336 | C = 0.364 | | | |
| 2286068 | 52245 | 71118395 | A/G | A = 0.639 | A = 0.618 | 0.52 | 0.92 | A |
| | | | | G = 0.361 | G = 0.382 | | | |
| 11339669 | 52594 | 71118744 | —/G | — = 0.001 | — = 0 | 0.944 | 0.00 | — |
| | | | | G = 0.999 | G = 1.000 | | | |
| 3854 | 52874 | 71119024 | A/G | A = 0.619 | A = 0.577 | 0.212 | 0.84 | A |
| | | | | G = 0.381 | G = 0.423 | | | |
| 11463 | 53011 | 71119161 | T/G | T = 0.59 | T = 0.591 | 0.963 | 1.01 | G |
| | | | | G = 0.410 | G = 0.409 | | | |
| 1383 | 53656 | 71119806 | A/T | A = 0.863 | A = 0.829 | 0.15 | 0.77 | A |
| | | | | T = 0.137 | T = 0.171 | | | |
| 1381 | 53662 | 71119812 | C/G | C = 0.001 | C = 0.001 | 0.966 | 0.18 | C |
| | | | | G = 0.999 | G = 0.999 | | | |
| 1377 | 53711 | 71119861 | T/C | T = 1.000 | T = 1.000 | 0.993 | 0.91 | T |
| | | | | C = 0.000 | C = 0.000 | | | |
| 11158966 | 53798 | 71119948 | A/G | A = 0 | A = 0.001 | 0.963 | | G |
| | | | | G = 1.000 | G = 0.999 | | | |
| 11158967 | 53840 | 71119990 | A/G | A = 0 | A = 0.001 | 0.963 | | G |
| | | | | G = 1.000 | G = 0.999 | | | |
| 11158968 | 53873 | 71120023 | C/T | C = 1.000 | C = 1.000 | 0.968 | 0.00 | C |
| | | | | T = 0 | T = 0.000 | | | |
| 11158969 | 53884 | 71120034 | A/G | A = 0 | A = 0.001 | 0.936 | | G |
| | | | | G = 1.000 | G = 0.999 | | | |
| 11158970 | 53937 | 71120087 | A/G | A = 0 | A = 0.001 | 0.979 | | G |
| | | | | G = 1.000 | G = 0.999 | | | |
| 11158971 | 54032 | 71120182 | G/A | G = 1.000 | G = 0.999 | 0.904 | 0.49 | G |
| | | | | A = 0.000 | A = 0.001 | | | |
| 11849782 | 54352 | 71120502 | C/G | C = 0.503 | C = 0.527 | 0.446 | 1.10 | G |
| | | | | G = 0.497 | G = 0.473 | | | |
| 10137616 | 55430 | 71121580 | C/T | C = 0.46 | C = 0.473 | 0.697 | 1.05 | T |
| | | | | T = 0.540 | T = 0.527 | | | |
| 757572 | 55920 | 71122070 | G/A | G = 0.614 | G = 0.579 | 0.27 | 0.87 | G |
| | | | | A = 0.386 | A = 0.421 | | | |
| 10140566 | 56081 | 71122231 | G/T | G = 0.493 | G = 0.517 | 0.464 | 1.10 | T |
| | | | | T = 0.507 | T = 0.483 | | | |
| 2023480 | 56227 | 71122377 | A/T | A = 0.984 | A = 0.999 | 0.282 | 13.67 | T |
| | | | | T = 0.016 | T = 0.001 | | | |
| 2526911 | 57350 | 71123500 | T/A | T = 0.029 | T = 0.008 | 0.322 | 0.27 | T |
| | | | | A = 0.971 | A = 0.992 | | | |
| 4903044 | 57493 | 71123643 | C/T | C = 0.603 | C = 0.566 | 0.239 | 0.86 | C |
| | | | | T = 0.397 | T = 0.434 | | | |
| 11845162 | 57682 | 71123832 | G/C | G = 1.000 | G = 1.000 | 1 | | |
| | | | | C = 0 | C = 0 | | | |
| 12147210 | 58092 | 71124242 | C/T | C = 1.000 | C = 0.996 | 0.771 | 0.18 | C |
| | | | | T = 0.000 | T = 0.004 | | | |
| 2997643 | 58731 | 71124881 | G/T | G = 1.000 | G = 1.000 | 0.946 | 0.00 | G |
| | | | | T = 0 | T = 0.000 | | | |
| 2997644 | 58826 | 71124976 | A/T | A = 1.000 | A = 1.000 | 0.992 | 0.47 | A |
| | | | | T = 0.000 | T = 0.000 | | | |
| 3814867 | 60916 | 71127066 | T/C | T = 0.999 | T = 0.999 | 0.982 | 0.82 | T |
| | | | | C = 0.001 | C = 0.001 | | | |
| 2286067 | 62047 | 71128197 | A/G | A = 0.999 | A = 1.000 | 0.951 | 2.09 | G |
| | | | | G = 0.001 | G = 0.000 | | | |
| 1060570 | 62080 | 71128230 | T/G | T = 0.469 | T = 0.494 | 0.414 | 1.11 | G |
| | | | | G = 0.531 | G = 0.506 | | | |
| 2332891 | 62665 | 71128815 | C/G | C = 1.000 | C = 1.000 | 0.997 | 1.14 | G |
| | | | | G = 0.000 | G = 0.000 | | | |
| 2021736 | 62809 | 71128959 | G/A | G = 0.995 | G = 0.994 | 0.897 | 0.81 | G |
| | | | | A = 0.005 | A = 0.006 | | | |
| 7160830 | 63629 | 71129779 | T/C | T = 0.518 | T = 0.542 | 0.461 | 1.10 | C |
| | | | | C = 0.482 | C = 0.458 | | | |
| 2526909 | 65432 | 71131582 | A/G | A = 0.998 | A = 1.000 | 0.823 | 5.46 | G |
| | | | | G = 0.002 | G = 0.000 | | | |
| 7140963 | 65590 | 71131740 | G/C | G = 1.000 | G = 1.000 | 0.959 | 2.57 | C |
| | | | | C = 0.000 | C = 0.000 | | | |
| 10143667 | 65876 | 71132026 | A/G | A = 0.058 | A = 0.053 | 0.823 | 0.91 | A |
| | | | | G = 0.942 | G = 0.947 | | | |
| 2803981 | 67118 | 71133268 | T/C | T = 0.685 | T = 0.686 | 0.96 | 1.01 | C |
| | | | | C = 0.315 | C = 0.314 | | | |
| 768840 | 67348 | 71133498 | T/C | T = 0.964 | T = 0.958 | 0.79 | 0.86 | T |
| | | | | C = 0.036 | C = 0.042 | | | |
| 5809587 | 68230 | 71134380 | T/— | T = 0.724 | T = 0.69 | 0.245 | 0.85 | T |
| | | | | — = 0.276 | — = 0.310 | | | |
| 2023479 | 68262 | 71134412 | A/G | A = 0.001 | A = 0.001 | 0.998 | 0.94 | A |
| | | | | G = 0.999 | G = 0.999 | | | |
| 4903045 | 68912 | 71135062 | T/C | T = 0.464 | T = 0.492 | 0.395 | 1.12 | C |
| | | | | C = 0.536 | C = 0.508 | | | |
| 4903046 | 69799 | 71135949 | A/G | A = 0.811 | A = 0.825 | 0.583 | 1.10 | G |
| | | | | G = 0.189 | G = 0.175 | | | |
| 5809588 | 70027 | 71136177 | —/G | — = 0.001 | — = 0.001 | 0.971 | 0.57 | — |
| | | | | G = 0.999 | G = 0.999 | | | |
| 10220555 | 73335 | 71139485 | G/A | G = 0.673 | G = 0.635 | 0.21 | 0.85 | G |
| | | | | A = 0.327 | A = 0.365 | | | |
| 2906132 | 74093 | 71140243 | G/A | G = 0.542 | G = 0.513 | 0.356 | 0.89 | G |
| | | | | A = 0.458 | A = 0.487 | | | |
| 11848482 | 74114 | 71140264 | T/G | T = 1.000 | T = 1.000 | 1 | | |
| | | | | G = 0 | G = 0 | | | |
| 10129954 | 74592 | 71140742 | T/C | T = 0.499 | T = 0.538 | 0.227 | 1.17 | C |
| | | | | C = 0.501 | C = 0.462 | | | |
| 2526921 | 75061 | 71141211 | C/T | C = 0.027 | C = 0.002 | 0.0158 | 0.05 | C |
| | | | | T = 0.973 | T = 0.998 | | | |
| 4903047 | 75683 | 71141833 | G/T | G = 0.48 | G = 0.493 | 0.708 | 1.05 | T |
| | | | | T = 0.520 | T = 0.507 | | | |
| 11846357 | 75857 | 71142007 | A/G | A = 1.000 | A = 0.999 | 0.91 | 0.31 | A |
| | | | | G = 0.000 | G = 0.001 | | | |
| 10136385 | 76457 | 71142607 | G/C | G = 0.515 | G = 0.533 | 0.583 | 1.07 | C |
| | | | | C = 0.485 | C = 0.467 | | | |
| 12588242 | 77130 | 71143280 | T/A | T = 0.001 | T = 0.001 | 0.978 | 0.61 | T |
| | | | | A = 0.999 | A = 0.999 | | | |
| 11621517 | 77947 | 71144097 | C/T | C = 0 | C = 0.002 | 0.912 | | T |
| | | | | T = 1.000 | T = 0.998 | | | |
| 2526910 | 78338 | 71144488 | C/T | C = 0.622 | C = 0.598 | 0.443 | 0.90 | C |
| | | | | T = 0.378 | T = 0.402 | | | |
| 2526941 | 79626 | 71145776 | A/G | A = 0.982 | A = 0.996 | 0.276 | 4.02 | G |
| | | | | G = 0.018 | G = 0.004 | | | |
| 2803944 | 80497 | 71146647 | G/A | G = 0.615 | G = 0.575 | 0.227 | 0.85 | G |
| | | | | A = 0.385 | A = 0.425 | | | |
| 12436770 | 80578 | 71146728 | A/G | A = 0.033 | A = 0.031 | 0.908 | 0.93 | A |
| | | | | G = 0.967 | G = 0.969 | | | |
| 10220279 | 81171 | 71147321 | C/A | C = 0.744 | C = 0.678 | 0.0671 | 0.72 | C |
| | | | | A = 0.256 | A = 0.322 | | | |
| 10220280 | 81243 | 71147393 | G/C | G = 0.001 | G = 0.003 | 0.833 | 4.04 | C |
| | | | | C = 0.999 | C = 0.997 | | | |
| 11844112 | 81321 | 71147471 | A/C | A = 0.001 | A = 0.002 | 0.924 | 2.92 | C |
| | | | | C = 0.999 | C = 0.998 | | | |
| 12432455 | 82634 | 71148784 | G/C | G = 0.57 | G = 0.575 | 0.877 | 1.02 | C |
| | | | | C = 0.430 | C = 0.425 | | | |
| 8015338 | 83269 | 71149419 | G/A | G = 1.000 | G = 0.999 | 0.891 | 0.00 | G |
| | | | | A = 0 | A = 0.001 | | | |
| 3832969 | 83514 | 71149664 | CAGC/— | CAGC = 0.003 | CAGC = 0.004 | 0.965 | 1.12 | — |
| | | | | — = 0.997 | — = 0.996 | | | |
| 9671972 | 85367 | 71151517 | A/G | A = 1.000 | A = 1.000 | 0.974 | 0.49 | A |
| | | | | G = 0.000 | G = 0.000 | | | |
| 9671975 | 85423 | 71151573 | C/T | C = 0.998 | C = 0.998 | 0.95 | 1.37 | T |
| | | | | T = 0.002 | T = 0.002 | | | |
| 7159986 | 86006 | 71152156 | T/C | T = 0.166 | T = 0.104 | 0.00774 | 0.58 | T |
| | | | | C = 0.834 | C = 0.896 | | | |
| 11848220 | 86515 | 71152665 | T/G | T = 1.000 | T = 1.000 | 0.978 | 0.00 | T |
| | | | | G = 0 | G = 0.000 | | | |
| 5016369 | 86936 | 71153086 | G/C | G = 0 | G = 0.001 | 0.99 | | C |
| | | | | C = 1.000 | C = 0.999 | | | |
| 1986423 | 87201 | 71153351 | A/G | A = 0.999 | A = 0.999 | 0.964 | 0.79 | A |
| | | | | G = 0.001 | G = 0.001 | | | |
| 2332909 | 87205 | 71153355 | C/T | C = 0.573 | C = 0.513 | 0.13 | 0.78 | C |
| | | | | T = 0.427 | T = 0.487 | | | |
| 8013337 | 88313 | 71154463 | G/A | G = 0.62 | G = 0.576 | 0.148 | 0.83 | G |
| | | | | A = 0.380 | A = 0.424 | | | |
| 11445680 | 89477 | 71155627 | —/G | — = 0.001 | — = 0.001 | 0.969 | 0.32 | — |
| | | | | G = 0.999 | G = 0.999 | | | |
| 4903048 | 90243 | 71156393 | T/C | T = 0.821 | T = | | | |
| | | | | C = 0.179 | C = | | | |
| 12434793 | 90735 | 71156885 | T/C | T = 0.688 | T = 0.651 | 0.223 | 0.85 | T |
| | | | | C = 0.312 | C = 0.349 | | | |
| 2332910 | 91990 | 71158140 | G/C | G = 0.003 | G = 0.002 | 0.893 | 0.49 | G |
| | | | | C = 0.997 | C = 0.998 | | | |
| 4410017 | 92879 | 71159029 | A/G | A = 0.003 | A = 0.001 | 0.872 | 0.33 | A |
| | | | | G = 0.997 | G = 0.999 | | | |
| 4577019 | 92880 | 71159030 | A/G | A = 1.000 | A = 0.999 | 0.917 | 0.00 | A |
| | | | | G = 0 | G = 0.001 | | | |
| 2332911 | 93386 | 71159536 | A/G | A = 0.996 | A = 0.995 | 0.97 | 0.90 | A |
| | | | | G = 0.004 | G = 0.005 | | | |
| 4996747 | 93397 | 71159547 | T/G | T = 0.999 | T = 0.999 | 0.984 | 0.84 | T |
| | | | | G = 0.001 | G = 0.001 | | | |
| 10140820 | 93680 | 71159830 | G/T | G = 0.002 | G = 0.001 | 0.94 | 0.46 | G |
| | | | | T = 0.998 | T = 0.999 | | | |
| 12431623 | 95585 | 71161735 | T/C | T = 0.995 | T = 0.995 | 0.976 | 1.05 | C |
| | | | | C = 0.005 | C = 0.005 | | | |
| 6574090 | 95818 | 71161968 | G/T | G = 0.001 | G = 0.001 | 0.966 | 2.92 | T |
| | | | | T = 0.999 | T = 0.999 | | | |
| 8008750 | 96203 | 71162353 | C/G | C = 0.576 | C = 0.579 | 0.92 | 1.01 | G |
| | | | | G = 0.424 | G = 0.421 | | | |
| 11627420 | 96911 | 71163061 | G/A | G = 0.986 | G = 0.988 | 0.878 | 1.14 | A |
| | | | | A = 0.014 | A = 0.012 | | | |
| 10150185 | 98014 | 71164164 | A/G | A = 0.934 | A = 0.931 | 0.877 | 0.96 | A |
| | | | | G = 0.066 | G = 0.069 | | | |
| 10150218 | 98229 | 71164379 | A/C | A = 0.724 | A = 0.723 | 0.993 | 1.00 | A |
| | | | | C = 0.276 | C = 0.277 | | | |
| 9646164 | 98235 | 71164385 | T/G | T = 0.797 | T = 0.772 | 0.351 | 0.86 | T |
| | | | | G = 0.203 | G = 0.228 | | | |
| 6574091 | 99173 | 71165323 | C/T | C = 0.995 | C = 0.999 | 0.652 | 3.38 | T |
| | | | | T = 0.005 | T = 0.001 | | | |
| 2332912 | 100505 | 71166655 | A/G | A = 0 | A = 0.001 | 0.941 | | G |
| | | | | G = 1.000 | G = 0.999 | | | |
| 7155359 | 101613 | 71167763 | A/G | A = 0.558 | A = 0.58 | 0.497 | 1.09 | G |
| | | | | G = 0.442 | G = 0.420 | | | |
| 2332913 | 101660 | 71167810 | C/T | C = 0.094 | C = 0.1 | 0.778 | 1.06 | T |
| | | | | T = 0.906 | T = 0.900 | | | |
| 4544184 | 101681 | 71167831 | G/T | G = 0.001 | G = 0.001 | 0.976 | 2.43 | T |
| | | | | T = 0.999 | T = 0.999 | | | |
| 4337224 | 102165 | 71168315 | G/A | G = 0.999 | G = 0.999 | 0.933 | 0.63 | G |
| | | | | A = 0.001 | A = 0.001 | | | |
| 2332914 | 103763 | 71169913 | C/T | C = 0.914 | C = 0.904 | 0.627 | 0.88 | C |
| | | | | T = 0.086 | T = 0.096 | | | |
| 4280167 | 104990 | 71171140 | G/A | G = 1.000 | G = 1.000 | 0.953 | 0.00 | G |
| | | | | A = 0 | A = 0.000 | | | |
| 4903049 | 105319 | 71171469 | G/T | G = 1.000 | G = 1.000 | 0.99 | 0.00 | G |
| | | | | T = 0 | T = 0.000 | | | |
| 3742837 | 105447 | 71171597 | T/G | T = 0.873 | T = 0.881 | 0.72 | 1.08 | G |
| | | | | G = 0.127 | G = 0.119 | | | |
| 10139937 | 105471 | 71171621 | A/G | A = 0.927 | A = 0.954 | 0.116 | 1.63 | G |
| | | | | G = 0.073 | G = 0.046 | | | |
| 12434292 | 105515 | 71171665 | A/G | A = 1.000 | A = 1.000 | 0.978 | 0.00 | A |
| | | | | G = 0 | G = 0.000 | | | |
| 11620671 | 105598 | 71171748 | A/T | A = 0.001 | A = 0.002 | 0.907 | 7.24 | T |
| | | | | T = 0.999 | T = 0.998 | | | |
| 10140184 | 105674 | 71171824 | C/A | C = 0.488 | C = 0.545 | 0.0731 | 1.26 | A |
| | | | | A = 0.512 | A = 0.455 | | | |
| 2332915 | 106432 | 71172582 | C/G | C = 0.45 | C = 0.461 | 0.749 | 1.04 | G |
| | | | | G = 0.550 | G = 0.539 | | | |
| 2332916 | 106988 | 71173138 | C/T | C = 0.245 | C = 0.19 | 0.0373 | 0.72 | C |
| | | | | T = 0.755 | T = 0.810 | | | |
| 8012759 | 107076 | 71173226 | C/T | C = 0.603 | C = 0.575 | 0.37 | 0.89 | C |
| | | | | T = 0.397 | T = 0.425 | | | |
| 10149272 | 107991 | 71174141 | G/C | G = 0.701 | G = 0.682 | 0.509 | 0.91 | G |
| | | | | C = 0.299 | C = 0.318 | | | |
| 11158973 | 108221 | 71174371 | A/G | A = 0.767 | A = 0.767 | 0.983 | 1.00 | G |
| | | | | G = 0.233 | G = 0.233 | | | |
| 4903050 | 108410 | 71174560 | G/T | G = 0.494 | G = 0.513 | 0.627 | 1.08 | T |
| | | | | T = 0.506 | T = 0.487 | | | |
| 10666445 | 108762 | 71174912 | CTTA/— | CTTA = | CTTA = 0.19 | | | |
| | | | | — = | — = 0.810 | | | |
| 2536143 | 108924 | 71175074 | A/T | A = 0.939 | A = 0.904 | 0.154 | 0.61 | A |
| | | | | T = 0.061 | T = 0.096 | | | |
| 10638231 | 109378 | 71175528 | TTTTTTT | TTTTTTTTT | TTTTTTTT | 0.909 | 1.06 | — |
| | | | TTTTTT/— | TTTT = 0.923 | TTTTT = | | | |
| | | | (SEQ ID | (SEQ ID NO: | 0.927 | | | |
| | | | NO: 4934) | 4934) | (SEQ ID NO: | | | |
| | | | | | 4934) | | | |
| | | | | — = 0.077 | — = 0.073 | | | |
| 12431636 | 109442 | 71175592 | A/G | A = 0.219 | A = 0.218 | 0.964 | 0.99 | A |
| | | | | G = 0.781 | G = 0.782 | | | |
| 8005904 | 109619 | 71175769 | C/T | C = | C = 0.002 | | | |
| | | | | T = | T = 0.998 | | | |
| 11621058 | 109820 | 71175970 | C/T | C = 0.881 | C = 0.863 | 0.417 | 0.85 | C |
| | | | | T = 0.119 | T = 0.137 | | | |
| 10133224 | 110530 | 71176680 | T/C | T = 0 | T = 0 | 1 | | |
| | | | | C = 1.000 | C = 1.000 | | | |
| 12433185 | 110956 | 71177106 | A/T | A = 0.692 | A = 0.679 | 0.674 | 0.94 | A |
| | | | | T = 0.308 | T = 0.321 | | | |
| 740974 | 111965 | 71178115 | A/G | A = 0.764 | A = 0.754 | 0.717 | 0.95 | A |
| | | | | G = 0.236 | G = 0.246 | | | |
| 740973 | 111995 | 71178145 | G/C | G = 0.435 | G = 0.468 | 0.315 | 1.14 | C |
| | | | | C = 0.565 | C = 0.532 | | | |
| 740972 | 112040 | 71178190 | T/C | T = 0.637 | T = 0.649 | 0.71 | 1.05 | C |
| | | | | C = 0.363 | C = 0.351 | | | |
| 740971 | 112058 | 71178208 | A/G | A = 0.978 | A = 0.977 | 0.962 | 0.97 | A |
| | | | | G = 0.022 | G = 0.023 | | | |
| 4243642 | 112592 | 71178742 | C/G | C = 0.573 | C = 0.574 | 0.987 | 1.00 | G |
| | | | | G = 0.427 | G = 0.426 | | | |
| 4415952 | 113044 | 71179194 | C/T | C = 0.654 | C = 0.648 | 0.839 | 0.97 | C |
| | | | | T = 0.346 | T = 0.352 | | | |
| 2215589 | 113263 | 71179413 | G/A | G = 0.999 | G = 0.998 | 0.909 | 0.60 | G |
| | | | | A = 0.001 | A = 0.002 | | | |
| 4343211 | 113301 | 71179451 | T/C | T = 0 | T = 0.001 | 0.984 | | C |
| | | | | C = 1.000 | C = 0.999 | | | |
| 2536142 | 113513 | 71179663 | T/C | T = 0.87 | T = 0.86 | 0.682 | 0.92 | T |
| | | | | C = 0.130 | C = 0.140 | | | |
| 11293340 | 113639 | 71179789 | —/A | — = 0.002 | — = 0.002 | 0.995 | 0.97 | — |
| | | | | A = 0.998 | A = 0.998 | | | |
| 10151620 | 114092 | 71180242 | C/G | C = 0.625 | C = 0.615 | 0.757 | 0.96 | C |
| | | | | G = 0.375 | G = 0.385 | | | |
| 2286840 | 114432 | 71180582 | T/G | T = 0.999 | T = 0.998 | 0.943 | 0.66 | T |
| | | | | G = 0.001 | G = 0.002 | | | |
| 8023089 | 114643 | 71180793 | T/A | T = 0 | T = | | | |
| | | | | A = 1.000 | A = | | | |
| 8008009 | 114980 | 71181130 | G/A | G = 0.616 | G = 0.628 | 0.693 | 1.05 | A |
| | | | | A = 0.384 | A = 0.372 | | | |
| 11419089 | 115312 | 71181462 | —/T | — = 0.755 | — = 0.743 | 0.661 | 0.93 | — |
| | | | | T = 0.245 | T = 0.257 | | | |
| 10694676 | 115315 | 71181465 | —/TT | — = 0.938 | — = 0.927 | 0.581 | 0.84 | — |
| | | | | TT = 0.062 | TT = 0.073 | | | |
| 2536141 | 115377 | 71181527 | C/T | C = 0.884 | C = 0.878 | 0.78 | 0.94 | C |
| | | | | T = 0.116 | T = 0.122 | | | |
| 765247 | 115625 | 71181775 | C/A | C = 0.978 | C = 0.981 | 0.917 | 1.13 | A |
| | | | | A = 0.022 | A = 0.019 | | | |
| 7147626 | 115740 | 71181890 | T/C | T = 0.973 | T = 0.95 | 0.147 | 0.53 | T |
| | | | | C = 0.027 | C = 0.050 | | | |
| 10132629 | 115747 | 71181897 | G/A | G = 0.649 | G = 0.656 | 0.799 | 1.03 | A |
| | | | | A = 0.351 | A = 0.344 | | | |
| 4903051 | 115890 | 71182040 | C/G | C = 0.976 | C = 0.965 | 0.541 | 0.68 | C |
| | | | | G = 0.024 | G = 0.035 | | | |
| 11158974 | 117279 | 71183429 | T/C | T = 0.976 | T = 0.961 | 0.241 | 0.60 | T |
| | | | | C = 0.024 | C = 0.039 | | | |
| 1035021 | 117522 | 71183672 | C/A | C = 0.513 | C = 0.509 | 0.909 | 0.99 | C |
| | | | | A = 0.487 | A = 0.491 | | | |
| 6574093 | 118721 | 71184871 | T/G | T = 0.433 | T = 0.445 | 0.721 | 1.05 | G |
| | | | | G = 0.567 | G = 0.555 | | | |
| 4899439 | 119926 | 71186076 | T/C | T = 0.973 | T = 0.958 | 0.35 | 0.65 | T |
| | | | | C = 0.027 | C = 0.042 | | | |
| 7155287 | 119974 | 71186124 | T/A | T = 1.000 | T = 0.999 | 0.835 | 0.17 | T |
| | | | | A = 0.000 | A = 0.001 | | | |
| 7154402 | 120281 | 71186431 | A/G | A = 0.972 | A = 0.948 | 0.158 | 0.53 | A |
| | | | | G = 0.028 | G = 0.052 | | | |
| 7154781 | 120484 | 71186634 | G/A | G = 0.971 | G = 0.956 | 0.315 | 0.65 | G |
| | | | | A = 0.029 | A = 0.044 | | | |
| 6574094 | 122397 | 71188547 | C/T | C = 0.972 | C = 0.948 | 0.0851 | 0.52 | C |
| | | | | T = 0.028 | T = 0.052 | | | |
| 10132242 | 122783 | 71188933 | G/A | G = 0.975 | G = 0.946 | 0.0436 | 0.46 | G |
| | | | | A = 0.025 | A = 0.054 | | | |
| 10146959 | 123029 | 71189179 | T/C | T = 0.694 | T = 0.698 | 0.875 | 1.02 | C |
| | | | | C = 0.306 | C = 0.302 | | | |
| 10147466 | 123535 | 71189685 | T/C | T = 1.000 | T = 1.000 | 0.95 | 0.00 | T |
| | | | | C = 0 | C = 0.000 | | | |
| 11626411 | 124107 | 71190257 | G/C | G = 0.757 | G = 0.774 | 0.539 | 1.10 | C |
| | | | | C = 0.243 | C = 0.226 | | | |
| 4899440 | 124427 | 71190577 | C/T | C = 0.683 | C = 0.727 | 0.129 | 1.24 | T |
| | | | | T = 0.317 | T = 0.273 | | | |
| 10144553 | 124738 | 71190888 | T/C | T = 0.539 | T = 0.526 | 0.677 | 0.95 | T |
| | | | | C = 0.461 | C = 0.474 | | | |
| 11627663 | 125329 | 71191479 | A/C | A = 0.009 | A = 0.078 | 0.00136 | 9.35 | C |
| | | | | C = 0.991 | C = 0.922 | | | |
| 11627799 | 125796 | 71191946 | A/G | A = 0.669 | A = 0.668 | 0.959 | 0.99 | A |
| | | | | G = 0.331 | G = 0.332 | | | |
| 11851733 | 126265 | 71192415 | G/A | G = 0.65 | G = 0.665 | 0.631 | 1.07 | A |
| | | | | A = 0.350 | A = 0.335 | | | |
| 10140317 | 126768 | 71192918 | A/T | A = 0.476 | A = 0.455 | 0.514 | 0.92 | A |
| | | | | T = 0.524 | T = 0.545 | | | |
| 8007375 | 127306 | 71193456 | C/T | C = 0.001 | C = 0.001 | 0.937 | 3.61 | T |
| | | | | T = 0.999 | T = 0.999 | | | |
| 4899441 | 127544 | 71193694 | G/A | G = 0.043 | G = 0.094 | 0.00298 | 2.32 | A |
| | | | | A = 0.957 | A = 0.906 | | | |
| 11158975 | 127809 | 71193959 | A/C | A = 0.798 | A = 0.814 | 0.564 | 1.11 | C |
| | | | | C = 0.202 | C = 0.186 | | | |
| 11419607 | 127817 | 71193967 | A/— | A = 0.829 | A = 0.808 | 0.406 | 0.86 | A |
| | | | | — = 0.171 | — = 0.192 | | | |
| 2109793 | 129771 | 71195921 | C/G | C = | C = 0.997 | | | |
| | | | | G = | G = 0.003 | | | |
| 8023156 | 130061 | 71196211 | G/C | G = 0.706 | G = 0.706 | 0.987 | 1.00 | C |
| | | | | C = 0.294 | C = 0.294 | | | |
| 10140579 | 130333 | 71196483 | G/A | G = 0.002 | G = 0.001 | 0.984 | 0.86 | G |
| | | | | A = 0.998 | A = 0.999 | | | |
| 7148199 | 131734 | 71197884 | T/C | T = 0.078 | T = 0.119 | 0.0481 | 1.61 | C |
| | | | | C = 0.922 | C = 0.881 | | | |
| 7146916 | 131787 | 71197937 | G/A | G = 0.001 | G = 0.002 | 0.823 | 54.34 | A |
| | | | | A = 0.999 | A = 0.998 | | | |
| 1990439 | 132633 | 71198783 | G/A | G = 0.67 | G = 0.72 | 0.103 | 1.27 | A |
| | | | | A = 0.330 | A = 0.280 | | | |
| 6574095 | 135279 | 71201429 | C/G | C = 0.057 | C = 0.093 | 0.0585 | 1.71 | G |
| | | | | G = 0.943 | G = 0.907 | | | |
| 10140037 | 135709 | 71201859 | T/A | T = 0.456 | T = 0.438 | 0.569 | 0.93 | T |
| | | | | A = 0.544 | A = 0.562 | | | |
| 8007249 | 136208 | 71202358 | A/G | A = 0.81 | A = 0.791 | 0.512 | 0.89 | A |
| | | | | G = 0.190 | G = 0.209 | | | |
| 1990438 | 137091 | 71203241 | A/G | A = 0.902 | A = 0.859 | 0.07 | 0.66 | A |
| | | | | G = 0.098 | G = 0.141 | | | |
| 1990437 | 137499 | 71203649 | T/C | T = 0.092 | T = 0.155 | 0.00367 | 1.82 | C |
| | | | | C = 0.908 | C = 0.845 | | | |
| 10146484 | 137705 | 71203855 | A/G | A = 0.69 | A = 0.679 | 0.719 | 0.95 | A |
| | | | | G = 0.310 | G = 0.321 | | | |
| 11627333 | 137726 | 71203876 | A/C | A = 0.336 | A = 0.325 | 0.716 | 0.95 | A |
| | | | | C = 0.664 | C = 0.675 | | | |
| 7155802 | 137944 | 71204094 | G/A | G = 0.012 | G = 0.007 | 0.746 | 0.55 | G |
| | | | | A = 0.988 | A = 0.993 | | | |
| 2109792 | 138175 | 71204325 | T/G | T = 0 | T = 0 | 1 | | |
| | | | | G = 1.000 | G = 1.000 | | | |
| 11429363 | 138224 | 71204374 | A/— | A = 0.24 | A = 0.262 | 0.455 | 1.13 | — |
| | | | | — = 0.760 | — = 0.738 | | | |
| 8004210 | 139319 | 71205469 | A/G | A = 0.94 | A = 0.887 | 0.00878 | 0.50 | A |
| | | | | G = 0.060 | G = 0.113 | | | |
| 2098194 | 139409 | 71205559 | C/T | C = 1.000 | C = 0.999 | 0.922 | 0.54 | C |
| | | | | T = 0.000 | T = 0.001 | | | |
| 2109791 | 139597 | 71205747 | T/G | T = 0.753 | T = 0.724 | 0.315 | 0.86 | T |
| | | | | G = 0.247 | G = 0.276 | | | |
| 11381729 | 140476 | 71206626 | —/A | — = 0.45 | — = 0.492 | 0.18 | 1.18 | A |
| | | | | A = 0.550 | A = 0.508 | | | |
| 1160094 | 140808 | 71206958 | C/T | C = 0.731 | C = 0.751 | 0.514 | 1.11 | T |
| | | | | T = 0.269 | T = 0.249 | | | |
| 11628051 | 141363 | 71207513 | A/T | A = 0.386 | A = 0.328 | 0.0555 | 0.77 | A |
| | | | | T = 0.614 | T = 0.672 | | | |
| 10136771 | 141655 | 71207805 | G/A | G = 0.341 | G = 0.38 | 0.235 | 1.18 | A |
| | | | | A = 0.659 | A = 0.620 | | | |
| 10139903 | 142715 | 71208865 | A/C | A = 0.592 | A = 0.547 | 0.145 | 0.83 | A |
| | | | | C = 0.408 | C = 0.453 | | | |
| 4903053 | 143253 | 71209403 | C/T | C = 0.74 | C = 0.768 | 0.327 | 1.16 | T |
| | | | | T = 0.260 | T = 0.232 | | | |
| 3742836 | 143911 | 71210061 | T/C | T = 0.752 | T = 0.776 | 0.39 | 1.14 | C |
| | | | | C = 0.248 | C = 0.224 | | | |
| 3742835 | 144044 | 71210194 | G/A | G = 0.311 | G = 0.311 | 0.999 | 1.00 | G |
| | | | | A = 0.689 | A = 0.689 | | | |
| 8003233 | 144670 | 71210820 | T/G | T = 0.002 | T = 0.002 | 0.971 | 0.78 | T |
| | | | | G = 0.998 | G = 0.998 | | | |
| 11621588 | 144961 | 71211111 | C/T | C = 0.999 | C = 1.000 | 0.913 | 2.87 | T |
| | | | | T = 0.001 | T = 0.000 | | | |
| 740981 | 146613 | 71212763 | G/C | G = 0.468 | G = 0.425 | 0.187 | 0.84 | G |
| | | | | C = 0.532 | C = 0.575 | | | |
| 8019492 | 147413 | 71213563 | C/A | C = 0.001 | C = 0.002 | 0.882 | 2.59 | A |
| | | | | A = 0.999 | A = 0.998 | | | |
| 8019644 | 147464 | 71213614 | G/A | G = 0.001 | G = 0.002 | 0.899 | 2.47 | A |
| | | | | A = 0.999 | A = 0.998 | | | |
| 8019646 | 147468 | 71213618 | G/A | G = 0.541 | G = 0.569 | 0.422 | 1.12 | A |
| | | | | A = 0.459 | A = 0.431 | | | |
| 8019653 | 147479 | 71213629 | A/G | A = 0.581 | A = 0.65 | 0.0322 | 1.34 | G |
| | | | | G = 0.419 | G = 0.350 | | | |
| 11450794 | 147488 | 71213638 | A/— | A = 0.002 | A = 0.001 | 0.946 | 0.50 | A |
| | | | | — = 0.998 | — = 0.999 | | | |
| 11348193 | 147500 | 71213650 | —/A | — = 0.67 | — = 0.636 | 0.277 | 0.86 | — |
| | | | | A = 0.330 | A = 0.364 | | | |
| 11450793 | 147527 | 71213677 | G/— | G = 0.007 | G = 0.005 | 0.894 | 0.77 | G |
| | | | | — = 0.993 | — = 0.995 | | | |
| 11282735 | 147534 | 71213684 | AAGAGA | AAGAGAAG | AAGAGAA | 0.911 | 0.59 | AAGAGAAGAG |
| | | | AGAG/— | AG = 0.004 | GAG = | | | (SEQ ID NO: |
| | | | (SEQ ID | (SEQ ID NO: | 0.003 | | | 4935) |
| | | | NO: 4935) | 4935) | (SEQ ID NO: | | | |
| | | | | | 4935) | | | |
| | | | | — = 0.996 | — = 0.997 | | | |
| 2191823 | 149573 | 71215723 | G/T | G = 0.504 | G = 0.467 | 0.242 | 0.86 | G |
| | | | | T = 0.496 | T = 0.533 | | | |
| 12232220 | 150798 | 71216948 | A/G | A = 0.143 | A = 0.235 | 0.000383 | 1.85 | G |
| | | | | G = 0.857 | G = 0.765 | | | |
| 11158976 | 154169 | 71220319 | C/T | C = 0.747 | C = 0.791 | 0.111 | 1.28 | T |
| | | | | T = 0.253 | T = 0.209 | | | |
| 4903054 | 155056 | 71221206 | C/T | C = 1.000 | C = 0.999 | 0.94 | 0.53 | C |
| | | | | T = 0.000 | T = 0.001 | | | |
| 11846755 | 155494 | 71221644 | T/C | T = 0.703 | T = 0.704 | 0.988 | 1.00 | C |
| | | | | C = 0.297 | C = 0.296 | | | |
| 12431988 | 157756 | 71223906 | G/A | G = 0.547 | G = 0.557 | 0.754 | 1.04 | A |
| | | | | A = 0.453 | A = 0.443 | | | |
| 12100526 | 157830 | 71223980 | T/C | T = 0.001 | T = 0.002 | 0.97 | 1.30 | C |
| | | | | C = 0.999 | C = 0.998 | | | |
| 11622568 | 158379 | 71224529 | A/C | A = 0.476 | A = 0.442 | 0.279 | 0.87 | A |
| | | | | C = 0.524 | C = 0.558 | | | |
| 9671230 | 159906 | 71226056 | C/T | C = 0.001 | C = 0.001 | 0.96 | 1.81 | T |
| | | | | T = 0.999 | T = 0.999 | | | |
| 12147487 | 159951 | 71226101 | A/T | A = 0.596 | A = 0.616 | 0.527 | 1.09 | T |
| | | | | T = 0.404 | T = 0.384 | | | |
| 4903055 | 160135 | 71226285 | T/C | T = 0.879 | T = 0.876 | 0.885 | 0.97 | T |
| | | | | C = 0.121 | C = 0.124 | | | |
| 12147515 | 160209 | 71226359 | G/A | G = 0.955 | G = 0.954 | 0.958 | 0.98 | G |
| | | | | A = 0.045 | A = 0.046 | | | |
| 11625092 | 160351 | 71226501 | C/T | C = 0.999 | C = 0.999 | 0.972 | 0.84 | C |
| | | | | T = 0.001 | T = 0.001 | | | |
| 4899442 | 160835 | 71226985 | T/G | T = 0.042 | T = 0.088 | 0.0124 | 2.21 | G |
| | | | | G = 0.958 | G = 0.912 | | | |
| 2052146 | 161110 | 71227260 | A/C | A = 0.977 | A = 0.957 | 0.145 | 0.53 | A |
| | | | | C = 0.023 | C = 0.043 | | | |
| 10220553 | 161346 | 71227496 | A/T | A = 0.438 | A = 0.45 | 0.714 | 1.05 | T |
| | | | | T = 0.562 | T = 0.550 | | | |
| 10220565 | 161670 | 71227820 | A/G | A = | A = 0.159 | | | |
| | | | | G = | G = 0.841 | | | |
| 2332917 | 163447 | 71229597 | G/C | G = 0.045 | G = 0.013 | 0.0175 | 0.27 | G |
| | | | | C = 0.955 | C = 0.987 | | | |
| 7158910 | 163479 | 71229629 | G/A | G = 1.000 | G = 1.000 | 0.988 | 0.00 | G |
| | | | | A = 0 | A = 0.000 | | | |
| 8015749 | 164377 | 71230527 | A/G | A = 0.203 | A = 0.303 | 0.00603 | 1.70 | G |
| | | | | G = 0.797 | G = 0.697 | | | |
| 8006812 | 166328 | 71232478 | A/G | A = 0.999 | A = 0.999 | 0.969 | 1.34 | G |
| | | | | G = 0.001 | G = 0.001 | | | |
| 8008338 | 166361 | 71232511 | C/T | C = 0.002 | C = 0.003 | 0.911 | 1.49 | T |
| | | | | T = 0.998 | T = 0.997 | | | |
| 2052145 | 167003 | 71233153 | T/G | T = 0.142 | T = 0.199 | 0.117 | 1.50 | G |
| | | | | G = 0.858 | G = 0.801 | | | |
| 2052144 | 167036 | 71233186 | T/G | T = 0.243 | T = 0.223 | 0.595 | 0.89 | T |
| | | | | G = 0.757 | G = 0.777 | | | |
| 10146692 | 167759 | 71233909 | A/G | A = 0.492 | A = 0.472 | 0.553 | 0.93 | A |
| | | | | G = 0.508 | G = 0.528 | | | |
| 11627305 | 167829 | 71233979 | A/G | A = 0.831 | A = 0.836 | 0.828 | 1.04 | G |
| | | | | G = 0.169 | G = 0.164 | | | |
| 2079989 | 168360 | 71234510 | C/T | C = 0.466 | C = 0.444 | 0.491 | 0.92 | C |
| | | | | T = 0.534 | T = 0.556 | | | |
| 8018881 | 168582 | 71234732 | C/A | C = 0.998 | C = 0.994 | 0.738 | 0.33 | C |
| | | | | A = 0.002 | A = 0.006 | | | |
| 4561387 | 169462 | 71235612 | A/T | A = 0.894 | A = 0.892 | 0.921 | 0.98 | A |
| | | | | T = 0.106 | T = 0.108 | | | |
| 11622025 | 170317 | 71236467 | A/C | A = 0.26 | A = 0.241 | 0.498 | 0.90 | A |
| | | | | C = 0.740 | C = 0.759 | | | |
| 740980 | 170669 | 71236819 | A/G | A = 0.378 | A = 0.354 | 0.498 | 0.90 | A |
| | | | | G = 0.622 | G = 0.646 | | | |
| 12433250 | 171295 | 71237445 | T/C | T = 0.285 | T = 0.258 | 0.36 | 0.87 | T |
| | | | | C = 0.715 | C = 0.742 | | | |
| 5809590 | 171395 | 71237545 | —/A | — = 0.249 | — = 0.266 | 0.554 | 1.09 | A |
| | | | | A = 0.751 | A = 0.734 | | | |
| 758915 | 171431 | 71237581 | T/C | T = 0.28 | T = 0.281 | 0.988 | 1.00 | C |
| | | | | C = 0.720 | C = 0.719 | | | |
| 758914 | 171626 | 71237776 | A/T | A = 0.249 | A = 0.249 | 0.998 | 1.00 | T |
| | | | | T = 0.751 | T = 0.751 | | | |
| 7154229 | 171735 | 71237885 | G/A | G = 1.000 | G = 0.999 | 0.901 | 0.17 | G |
| | | | | A = 0.000 | A = 0.001 | | | |
| 12437188 | 171972 | 71238122 | T/C | T = 0.894 | T = 0.948 | 0.00279 | 2.15 | C |
| | | | | C = 0.106 | C = 0.052 | | | |
| 7159108 | 172258 | 71238408 | T/A | T = 0.739 | T = 0.746 | 0.802 | 1.04 | A |
| | | | | A = 0.261 | A = 0.254 | | | |
| 4903058 | 172448 | 71238598 | G/C | G = 1.000 | G = 1.000 | 0.993 | | C |
| | | | | C = 0.000 | C = 0 | | | |
| 4899443 | 172918 | 71239068 | C/A | C = 0.775 | C = 0.806 | 0.264 | 1.21 | A |
| | | | | A = 0.225 | A = 0.194 | | | |
| 11848907 | 173415 | 71239565 | A/G | A = 0.037 | A = 0.032 | 0.704 | 0.85 | A |
| | | | | G = 0.963 | G = 0.968 | | | |
| 12432148 | 173467 | 71239617 | G/T | G = 0.201 | G = 0.11 | 0.000319 | 0.49 | G |
| | | | | T = 0.799 | T = 0.890 | | | |
| 12435078 | 173697 | 71239847 | C/T | C = 0.912 | C = 0.905 | 0.823 | 0.92 | C |
| | | | | T = 0.088 | T = 0.095 | | | |
| 6574096 | 173991 | 71240141 | A/G | A = 0.461 | A = 0.411 | 0.122 | 0.82 | A |
| | | | | G = 0.539 | G = 0.589 | | | |
| 11158977 | 174091 | 71240241 | T/A | T = 0.4 | T = 0.435 | 0.259 | 1.16 | A |
| | | | | A = 0.600 | A = 0.565 | | | |
| 11629434 | 174571 | 71240721 | G/T | G = 0.841 | G = 0.842 | 0.974 | 1.01 | T |
| | | | | T = 0.159 | T = 0.158 | | | |
| 4378563 | 177780 | 71243930 | C/T | C = 0.863 | C = 0.932 | 0.00102 | 2.16 | T |
| | | | | T = 0.137 | T = 0.068 | | | |
| 2098195 | 178129 | 71244279 | C/G | C = 0.032 | C = 0.03 | 0.906 | 0.95 | C |
| | | | | G = 0.968 | G = 0.970 | | | |
| 4633653 | 178693 | 71244843 | C/A | C = 0.998 | C = 0.998 | 0.931 | 1.34 | A |
| | | | | A = 0.002 | A = 0.002 | | | |
| 7148350 | 179325 | 71245475 | G/A | G = 0.962 | G = 0.953 | 0.535 | 0.80 | G |
| | | | | A = 0.038 | A = 0.047 | | | |
| 740979 | 179511 | 71245661 | A/T | A = 0.34 | A = 0.31 | 0.36 | 0.87 | A |
| | | | | T = 0.660 | T = 0.690 | | | |
| 740978 | 179608 | 71245758 | G/C | G = 0.983 | G = 0.953 | 0.02 | 0.35 | G |
| | | | | C = 0.017 | C = 0.047 | | | |
| 740977 | 179644 | 71245794 | A/G | A = 0.086 | A = 0.129 | 0.0512 | 1.57 | G |
| | | | | G = 0.914 | G = 0.871 | | | |
| 740976 | 179808 | 71245958 | T/C | T = 0.39 | T = 0.323 | 0.0353 | 0.74 | T |
| | | | | C = 0.610 | C = 0.677 | | | |
| 4903059 | 180041 | 71246191 | C/T | C = 0.138 | C = 0.083 | 0.00674 | 0.56 | C |
| | | | | T = 0.862 | T = 0.917 | | | |
| 4903060 | 181420 | 71247570 | G/A | G = 0.995 | G = 0.96 | 0.00327 | 0.13 | G |
| | | | | A = 0.005 | A = 0.040 | | | |
| 11852056 | 181423 | 71247573 | A/G | A = 0.001 | A = 0.001 | 0.999 | 1.03 | G |
| | | | | G = 0.999 | G = 0.999 | | | |
| 9635257 | 181932 | 71248082 | C/T | C = 0.947 | C = 0.954 | 0.722 | 1.17 | T |
| | | | | T = 0.053 | T = 0.046 | | | |
| 9635258 | 181992 | 71248142 | C/T | C = 0.93 | C = 0.935 | 0.83 | 1.09 | T |
| | | | | T = 0.070 | T = 0.065 | | | |
| 9635259 | 182248 | 71248398 | A/G | A = 0.896 | A = 0.942 | 0.0142 | 1.88 | G |
| | | | | G = 0.104 | G = 0.058 | | | |
| 4899444 | 182335 | 71248485 | G/A | G = 0.868 | G = 0.887 | 0.418 | 1.19 | A |
| | | | | A = 0.132 | A = 0.113 | | | |
| 12435382 | 184268 | 71250418 | G/A | G = 0.777 | G = 0.79 | 0.614 | 1.08 | A |
| | | | | A = 0.223 | A = 0.210 | | | |
| 12435412 | 184366 | 71250516 | G/A | G = 0.831 | G = 0.828 | 0.883 | 0.98 | G |
| | | | | A = 0.169 | A = 0.172 | | | |
| 11627381 | 184855 | 71251005 | G/A | G = 0.178 | G = 0.177 | 0.955 | 0.99 | G |
| | | | | A = 0.822 | A = 0.823 | | | |
| 2052143 | 185532 | 71251682 | G/A | G = 0.537 | G = 0.593 | 0.072 | 1.26 | A |
| | | | | A = 0.463 | A = 0.407 | | | |
| 2052142 | 185633 | 71251783 | G/A | G = 0.98 | G = 0.95 | 0.0255 | 0.39 | G |
| | | | | A = 0.020 | A = 0.050 | | | |
| 2052141 | 185717 | 71251867 | C/T | C = 0.641 | C = 0.686 | 0.134 | 1.22 | T |
| | | | | T = 0.359 | T = 0.314 | | | |
| 8021035 | 186637 | 71252787 | A/G | A = 0.001 | A = 0.002 | 0.955 | 1.61 | G |
| | | | | G = 0.999 | G = 0.998 | | | |
| 11158978 | 187010 | 71253160 | A/G | A = 0.402 | A = 0.387 | 0.666 | 0.94 | A |
| | | | | G = 0.598 | G = 0.613 | | | |
| 11621216 | 187367 | 71253517 | A/T | A = 0.882 | A = 0.882 | 0.979 | 0.99 | A |
| | | | | T = 0.118 | T = 0.118 | | | |
| 10130873 | 187524 | 71253674 | T/C | T = 0.901 | T = 0.936 | 0.0616 | 1.60 | C |
| | | | | C = 0.099 | C = 0.064 | | | |
| 11621358 | 187525 | 71253675 | G/A | G = 0.864 | G = 0.867 | 0.863 | 1.03 | A |
| | | | | A = 0.136 | A = 0.133 | | | |
| 758913 | 188352 | 71254502 | A/G | A = 0.071 | A = 0.133 | 0.00789 | 2.00 | G |
| | | | | G = 0.929 | G = 0.867 | | | |
| 11622602 | 188360 | 71254510 | A/G | A = 0.241 | A = 0.218 | 0.402 | 0.88 | A |
| | | | | G = 0.759 | G = 0.782 | | | |
| 10136931 | 189032 | 71255182 | C/T | C = 1.000 | C = 1.000 | 0.969 | 0.00 | C |
| | | | | T = 0 | T = 0.000 | | | |
| 740975 | 189100 | 71255250 | T/G | T = 0.543 | T = 0.489 | 0.116 | 0.80 | T |
| | | | | G = 0.457 | G = 0.511 | | | |
| 747987 | 189444 | 71255594 | T/C | T = 0.291 | T = 0.186 | 0.000134 | 0.56 | T |
| | | | | C = 0.709 | C = 0.814 | | | |
| 11623819 | 189450 | 71255600 | C/T | C = 0.736 | C = 0.703 | 0.285 | 0.85 | C |
| | | | | T = 0.264 | T = 0.297 | | | |
| 12434445 | 190431 | 71256581 | C/A | C = 0.003 | C = 0.001 | 0.88 | 0.44 | C |
| | | | | A = 0.997 | A = 0.999 | | | |
| 8003375 | 190813 | 71256963 | T/A | T = 0.001 | T = 0.001 | 0.995 | 1.58 | A |
| | | | | A = 0.999 | A = 0.999 | | | |
| 10143596 | 190983 | 71257133 | A/G | A = 0.997 | A = 0.996 | 0.925 | 0.75 | A |
| | | | | G = 0.003 | G = 0.004 | | | |
| 12435321 | 191493 | 71257643 | T/C | T = 0.164 | T = 0.163 | 0.968 | 0.99 | T |
| | | | | C = 0.836 | C = 0.837 | | | |
| 12435401 | 191588 | 71257738 | A/G | A = | A = 0.302 | | | |
| | | | | G = | G = 0.698 | | | |
| 8010609 | 191764 | 71257914 | T/C | T = 1.000 | T = 0.999 | 0.915 | 0.43 | T |
| | | | | C = 0.000 | C = 0.001 | | | |
| 11158979 | 191811 | 71257961 | G/C | G = 0.291 | G = 0.284 | 0.822 | 0.97 | G |
| | | | | C = 0.709 | C = 0.716 | | | |
| 11158980 | 192126 | 71258276 | C/T | C = 0.271 | C = 0.243 | 0.324 | 0.87 | C |
| | | | | T = 0.729 | T = 0.757 | | | |
| 11627524 | 192356 | 71258506 | C/A | C = 0.897 | C = 0.892 | 0.828 | 0.95 | C |
| | | | | A = 0.103 | A = 0.108 | | | |
| 1126160 | 192953 | 71259103 | A/C | A = 0.657 | A = 0.587 | 0.0717 | 0.74 | A |
| | | | | C = 0.343 | C = 0.413 | | | |
| 10136074 | 194133 | 71260283 | G/C | G = 0.998 | G = 0.993 | 0.564 | 0.28 | G |
| | | | | C = 0.002 | C = 0.007 | | | |
| 4903061 | 195333 | 71261483 | T/A | T = 0.372 | T = | | | |
| | | | | A = 0.628 | A = | | | |
| 10665398 | 195657 | 71261807 | —/CA | — = 0.538 | — = 0.544 | 0.842 | 1.03 | CA |
| | | | | CA = 0.462 | CA = 0.456 | | | |
| 6574097 | 195668 | 71261818 | A/C | A = 0.976 | A = 0.949 | 0.176 | 0.46 | A |
| | | | | C = 0.024 | C = 0.051 | | | |
| 9671654 | 195676 | 71261826 | C/G | C = 0.253 | C = 0.235 | 0.538 | 0.91 | C |
| | | | | G = 0.747 | G = 0.765 | | | |
| 10140024 | 195805 | 71261955 | C/T | C = 0.354 | C = 0.314 | 0.203 | 0.84 | C |
| | | | | T = 0.646 | T = 0.686 | | | |
| 10142421 | 195998 | 71262148 | G/A | G = 0.97 | G = 0.973 | 0.832 | 1.10 | A |
| | | | | A = 0.030 | A = 0.027 | | | |
| 10142593 | 196066 | 71262216 | C/T | C = 0.023 | C = 0.057 | 0.0509 | 2.57 | T |
| | | | | T = 0.977 | T = 0.943 | | | |
| 11423372 | 196415 | 71262565 | —/C | — = 0.072 | — = 0.113 | 0.0327 | 1.65 | C |
| | | | | C = 0.928 | C = 0.887 | | | |
| 5809592 | 196417 | 71262567 | —/C | — = 0.0680 | — = 0.109 | 0.0442 | 1.68 | C |
| | | | | C = 0.932 | C = 0.891 | | | |
| 2332918 | 196538 | 71262688 | C/T | C = 0.953 | C = 0.93 | 0.169 | 0.65 | C |
| | | | | T = 0.047 | T = 0.070 | | | |
| 2332919 | 196569 | 71262719 | T/C | T = 0.704 | T = 0.728 | 0.388 | 1.13 | C |
| | | | | C = 0.296 | C = 0.272 | | | |
| 8007348 | 196621 | 71262771 | A/C | A = 0.417 | A = 0.34 | 0.0161 | 0.72 | A |
| | | | | C = 0.583 | C = 0.660 | | | |
| 1990443 | 196806 | 71262956 | G/A | G = 0.678 | G = 0.729 | 0.0751 | 1.28 | A |
| | | | | A = 0.322 | A = 0.271 | | | |
| 8007690 | 196823 | 71262973 | C/G | C = 0.175 | C = 0.169 | 0.829 | 0.96 | C |
| | | | | G = 0.825 | G = 0.831 | | | |
| 3937455 | 197204 | 71263354 | G/C | G = 0.556 | G = 0.546 | 0.768 | 0.96 | G |
| | | | | C = 0.444 | C = 0.454 | | | |
| 8012823 | 197435 | 71263585 | C/T | C = 0.65 | C = 0.651 | 0.97 | 1.00 | T |
| | | | | T = 0.350 | T = 0.349 | | | |
| 4899445 | 197612 | 71263762 | G/A | G = 0.129 | G = 0.0630 | 0.000836 | 0.45 | G |
| | | | | A = 0.871 | A = 0.937 | | | |
| 4903062 | 197854 | 71264004 | C/T | C = 0.229 | C = 0.118 | 5.96E−06 | 0.45 | C |
| | | | | T = 0.771 | T = 0.882 | | | |
| 973963 | 198264 | 71264414 | G/A | G = 0.968 | G = 0.964 | 0.736 | 0.87 | G |
| | | | | A = 0.032 | A = 0.036 | | | |
| 10149962 | 198835 | 71264985 | T/G | T = 0.963 | T = 0.949 | 0.321 | 0.71 | T |
| | | | | G = 0.037 | G = 0.051 | | | |
| 8003449 | 199275 | 71265425 | A/G | A = 0.39 | A = 0.368 | 0.576 | 0.91 | A |
| | | | | G = 0.610 | G = 0.632 | | | |
| 11620775 | 199336 | 71265486 | C/T | C = 0.827 | C = 0.809 | 0.476 | 0.88 | C |
| | | | | T = 0.173 | T = 0.191 | | | |
| 8009089 | 199903 | 71266053 | A/G | A = 0.848 | A = 0.876 | 0.217 | 1.27 | G |
| | | | | G = 0.152 | G = 0.124 | | | |
| 8008967 | 200017 | 71266167 | T/C | T = 0.358 | T = 0.406 | 0.136 | 1.23 | C |
| | | | | C = 0.642 | C = 0.594 | | | |
| 12050132 | 200141 | 71266291 | A/T | A = 0.782 | A = 0.761 | 0.509 | 0.89 | A |
| | | | | T = 0.218 | T = 0.239 | | | |
| 10133018 | 200552 | 71266702 | T/C | T = 0.999 | T = 0.993 | 0.511 | 0.20 | T |
| | | | | C = 0.001 | C = 0.007 | | | |
| 1990442 | 200868 | 71267018 | T/C | T = | T = 0.926 | | | |
| | | | | C = | C = 0.074 | | | |
| 1990441 | 200983 | 71267133 | T/G | T = 0.873 | T = 0.848 | 0.338 | 0.81 | T |
| | | | | G = 0.127 | G = 0.152 | | | |
| 8015833 | 200990 | 71267140 | C/T | C = 0.768 | C = 0.79 | 0.423 | 1.14 | T |
| | | | | T = 0.232 | T = 0.210 | | | |
| 1990440 | 201045 | 71267195 | G/C | G = 0.258 | G = 0.161 | 0.000297 | 0.55 | G |
| | | | | C = 0.742 | C = 0.839 | | | |
| 4903063 | 202426 | 71268576 | C/G | C = 0.989 | C = 0.969 | 0.188 | 0.35 | C |
| | | | | G = 0.011 | G = 0.031 | | | |
| 10139810 | 202841 | 71268991 | C/A | C = 0.002 | C = 0.006 | 0.74 | 3.78 | A |
| | | | | A = 0.998 | A = 0.994 | | | |
| 4903064 | 203311 | 71269461 | T/C | T = 0.83 | T = 0.823 | 0.783 | 0.95 | T |
| | | | | C = 0.170 | C = 0.177 | | | |
| 2159715 | 203543 | 71269693 | A/C | A = 0.468 | A = 0.458 | 0.748 | 0.96 | A |
| | | | | C = 0.532 | C = 0.542 | | | |
| 2109795 | 203749 | 71269899 | A/G | A = 0.206 | A = 0.255 | 0.106 | 1.32 | G |
| | | | | G = 0.794 | G = 0.745 | | | |
| 2159714 | 204040 | 71270190 | G/A | G = 0.965 | G = 0.96 | 0.714 | 0.87 | G |
| | | | | A = 0.035 | A = 0.040 | | | |
| 11623107 | 204269 | 71270419 | A/C | A = 0.001 | A = 0.002 | 0.883 | 5.49 | C |
| | | | | C = 0.999 | C = 0.998 | | | |
| 7152005 | 204309 | 71270459 | C/T | C = 0.806 | C = 0.776 | 0.295 | 0.84 | C |
| | | | | T = 0.194 | T = 0.224 | | | |
| 7152489 | 204426 | 71270576 | A/G | A = 0.004 | A = 0.002 | 0.919 | 0.65 | A |
| | | | | G = 0.996 | G = 0.998 | | | |
| 4479167 | 205135 | 71271285 | G/C | G = 0.169 | G = 0.278 | 0.000123 | 1.90 | C |
| | | | | C = 0.831 | C = 0.722 | | | |
| 4606655 | 205269 | 71271419 | T/C | T = 0.794 | T = 0.734 | 0.041 | 0.72 | T |
| | | | | C = 0.206 | C = 0.266 | | | |
| 4606656 | 205323 | 71271473 | T/C | T = 0.938 | T = 0.872 | 0.0032 | 0.45 | T |
| | | | | C = 0.062 | C = 0.128 | | | |
| 4489943 | 205364 | 71271514 | A/G | A = 0.546 | A = 0.427 | 0.00025 | 0.62 | A |
| | | | | G = 0.454 | G = 0.573 | | | |
| 7142642 | 206310 | 71272460 | G/C | G = 0.616 | G = 0.601 | 0.643 | 0.94 | G |
| | | | | C = 0.384 | C = 0.399 | | | |
| 8006233 | 206600 | 71272750 | A/G | A = 0.814 | A = | | | |
| | | | | G = 0.186 | G = | | | |
| 8007559 | 206786 | 71272936 | T/A | T = 0.426 | T = 0.552 | 6.35E−05 | 1.66 | A |
| | | | | A = 0.574 | A = 0.448 | | | |
| 8006156 | 206796 | 71272946 | A/G | A = 0.456 | A = 0.583 | 0.000459 | 1.67 | G |
| | | | | G = 0.544 | G = 0.417 | | | |
| 1468662 | 207633 | 71273783 | A/G | A = 0.961 | A = 0.932 | 0.0674 | 0.56 | A |
| | | | | G = 0.039 | G = 0.068 | | | |
| 11627048 | 207723 | 71273873 | G/A | G = 0.791 | G = 0.781 | 0.714 | 0.94 | G |
| | | | | A = 0.209 | A = 0.219 | | | |
| 6574098 | 208026 | 71274176 | T/C | T = 0.122 | T = 0.152 | 0.199 | 1.29 | C |
| | | | | C = 0.878 | C = 0.848 | | | |
| 12433760 | 208627 | 71274777 | C/T | C = 0.695 | C = 0.668 | 0.462 | 0.88 | C |
| | | | | T = 0.305 | T = 0.332 | | | |
| 12433780 | 208662 | 71274812 | C/T | C = 0.102 | C = 0.072 | 0.107 | 0.68 | C |
| | | | | T = 0.898 | T = 0.928 | | | |
| 12434506 | 209109 | 71275259 | T/A | T = 0.906 | T = 0.946 | 0.0611 | 1.81 | A |
| | | | | A = 0.094 | A = 0.054 | | | |
| 12434673 | 209742 | 71275892 | T/C | T = 0.976 | T = 0.998 | 0.0508 | 10.27 | C |
| | | | | C = 0.024 | C = 0.002 | | | |
| 12431400 | 209750 | 71275900 | T/C | T = 0.335 | T = 0.233 | 0.00048 | 0.60 | T |
| | | | | C = 0.665 | C = 0.767 | | | |
| 12431384 | 209817 | 71275967 | G/A | G = 0.173 | G = 0.101 | 0.00147 | 0.54 | G |
| | | | | A = 0.827 | A = 0.899 | | | |
| 4903067 | 210191 | 71276341 | C/T | C = 0.449 | C = 0.408 | 0.192 | 0.85 | C |
| | | | | T = 0.551 | T = 0.592 | | | |
| 2332920 | 210349 | 71276499 | G/A | G = 0.57 | G = | | | |
| | | | | A = 0.430 | A = | | | |
| 2215591 | 210724 | 71276874 | A/G | A = 0.111 | A = 0.146 | 0.143 | 1.36 | G |
| | | | | G = 0.889 | G = 0.854 | | | |
| 12433064 | 211537 | 71277687 | C/A | C = 0.95 | C = 0.952 | 0.867 | 1.06 | A |
| | | | | A = 0.050 | A = 0.048 | | | |
| 12436263 | 211551 | 71277701 | T/G | T = 0.973 | T = 0.994 | 0.2 | 4.13 | G |
| | | | | G = 0.027 | G = 0.006 | | | |
| 12433097 | 211659 | 71277809 | G/A | G = 0.202 | G = 0.118 | 0.00184 | 0.53 | G |
| | | | | A = 0.798 | A = 0.882 | | | |
| 12433184 | 211757 | 71277907 | G/T | G = 0.854 | G = 0.92 | 0.0033 | 1.96 | T |
| | | | | T = 0.146 | T = 0.080 | | | |
| 8015900 | 211821 | 71277971 | A/T | A = 0.004 | A = 0.003 | 0.907 | 0.72 | A |
| | | | | T = 0.996 | T = 0.997 | | | |
| 11296981 | 212236 | 71278386 | T/— | T = 0.819 | T = 0.796 | 0.413 | 0.87 | T |
| | | | | — = 0.181 | — = 0.204 | | | |
| 11344025 | 212237 | 71278387 | T/— | T = 0.817 | T = 0.798 | 0.511 | 0.89 | T |
| | | | | — = 0.183 | — = 0.202 | | | |
| 11464204 | 212442 | 71278592 | —/A | — = 1.000 | — = 1.000 | 0.99 | 1.43 | A |
| | | | | A = 0.000 | A = 0.000 | | | |
| 11441172 | 212445 | 71278595 | —/T | — = 0.281 | — = 0.296 | 0.588 | 1.08 | T |
| | | | | T = 0.719 | T = 0.704 | | | |
| 11381807 | 212455 | 71278605 | T/— | T = 0.865 | T = 0.844 | 0.339 | 0.84 | T |
| | | | | — = 0.135 | — = 0.156 | | | |
| 10873251 | 212730 | 71278880 | G/A | G = 0.093 | G = 0.076 | 0.361 | 0.80 | G |
| | | | | A = 0.907 | A = 0.924 | | | |
| 2109794 | 212746 | 71278896 | T/C | T = 0.301 | T = 0.317 | 0.58 | 1.08 | C |
| | | | | C = 0.699 | C = 0.683 | | | |
| 10873252 | 212836 | 71278986 | T/C | T = 0.809 | T = 0.795 | 0.604 | 0.92 | T |
| | | | | C = 0.191 | C = 0.205 | | | |
| 4520784 | 212886 | 71279036 | G/A | G = 0 | G = | | | |
| | | | | A = 1.000 | A = | | | |
| 2877820 | 213028 | 71279178 | A/T | A = 0.005 | A = 0.002 | 0.7 | 0.30 | A |
| | | | | T = 0.995 | T = 0.998 | | | |
| 2877821 | 213029 | 71279179 | A/T | A = 0.222 | A = 0.138 | 0.000936 | 0.56 | A |
| | | | | T = 0.778 | T = 0.862 | | | |
| 4569195 | 213095 | 71279245 | C/G | C = 0.888 | C = 0.944 | 0.0116 | 2.11 | G |
| | | | | G = 0.112 | G = 0.056 | | | |
| 11628905 | 214247 | 71280397 | A/C | A = 0.008 | A = 0.060 | 0.0167 | 8.40 | C |
| | | | | C = 0.992 | C = 0.940 | | | |
| 2191822 | 214807 | 71280957 | T/C | T = 0.104 | T = 0.162 | 0.0117 | 1.67 | C |
| | | | | C = 0.896 | C = 0.838 | | | |
| 2191821 | 214921 | 71281071 | A/C | A = 0.574 | A = 0.582 | 0.794 | 1.04 | C |
| | | | | C = 0.426 | C = 0.418 | | | |
| 11464689 | 215163 | 71281313 | T/— | T = 0.52 | T = 0.484 | 0.254 | 0.86 | T |
| | | | | — = 0.480 | — = 0.516 | | | |
| 12431959 | 215406 | 71281556 | G/A | G = 0.747 | G = 0.74 | 0.82 | 0.97 | G |
| | | | | A = 0.253 | A = 0.260 | | | |
| 4903068 | 216069 | 71282219 | A/G | A = 0.457 | A = 0.492 | 0.273 | 1.15 | G |
| | | | | G = 0.543 | G = 0.508 | | | |
| 1544579 | 216849 | 71282999 | T/C | T = 0.507 | T = 0.522 | 0.64 | 1.06 | C |
| | | | | C = 0.493 | C = 0.478 | | | |
| 7155879 | 217130 | 71283280 | G/T | G = 0.942 | G = 0.971 | 0.0802 | 2.05 | T |
| | | | | T = 0.058 | T = 0.029 | | | |
| 12433603 | 217143 | 71283293 | C/G | C = 0.898 | C = 0.939 | 0.0273 | 1.75 | G |
| | | | | G = 0.102 | G = 0.061 | | | |
| 11351432 | 217657 | 71283807 | A/— | A = 0.261 | A = 0.287 | 0.387 | 1.14 | — |
| | | | | — = 0.739 | — = 0.713 | | | |
| 11448438 | 217725 | 71283875 | —/G | — = 0.022 | — = 0.092 | 0.000309 | 4.52 | G |
| | | | | G = 0.978 | G = 0.908 | | | |
| 4243643 | 218435 | 71284585 | G/A | G = 0.617 | G = 0.578 | 0.257 | 0.85 | G |
| | | | | A = 0.383 | A = 0.422 | | | |
| 4140952 | 218717 | 71284867 | T/C | T = 0.437 | T = 0.462 | 0.43 | 1.11 | C |
| | | | | C = 0.563 | C = 0.538 | | | |
| 8005372 | 218812 | 71284962 | C/T | C = 0 | C = 0.001 | 0.903 | | T |
| | | | | T = 1.000 | T = 0.999 | | | |
| 8004109 | 218826 | 71284976 | T/C | T = 0.002 | T = 0.002 | 0.966 | 0.77 | T |
| | | | | C = 0.998 | C = 0.998 | | | |
| 8008398 | 218872 | 71285022 | A/C | A = 0.616 | A = 0.569 | 0.181 | 0.82 | A |
| | | | | C = 0.384 | C = 0.431 | | | |
| 8009692 | 218890 | 71285040 | T/C | T = 0.817 | T = 0.744 | 0.0353 | 0.65 | T |
| | | | | C = 0.183 | C = 0.256 | | | |
| 2332921 | 218944 | 71285094 | C/G | C = 0.999 | C = 1.000 | 0.932 | 1.94 | G |
| | | | | G = 0.001 | G = 0.000 | | | |
| 12050320 | 219229 | 71285379 | A/G | A = 0 | A = 0.001 | 0.949 | | G |
| | | | | G = 1.000 | G = 0.999 | | | |
| 12050323 | 219300 | 71285450 | A/G | A = 0.001 | A = 0.001 | 0.967 | 0.68 | A |
| | | | | G = 0.999 | G = 0.999 | | | |
| 8010316 | 219757 | 71285907 | A/G | A = 0.62 | A = 0.607 | 0.703 | 0.95 | A |
| | | | | G = 0.380 | G = 0.393 | | | |
| 7154293 | 220533 | 71286683 | T/C | T = 0.551 | T = 0.558 | 0.837 | 1.03 | C |
| | | | | C = 0.449 | C = 0.442 | | | |
| 12147969 | 221534 | 71287684 | C/T | C = 0.857 | C = 0.844 | 0.604 | 0.90 | C |
| | | | | T = 0.143 | T = 0.156 | | | |
| 2215590 | 221632 | 71287782 | G/A | G = 0.732 | G = 0.712 | 0.543 | 0.91 | G |
| | | | | A = 0.268 | A = 0.288 | | | |
| 4265749 | 222234 | 71288384 | C/T | C = 0.942 | C = 0.992 | 0.0263 | 7.44 | T |
| | | | | T = 0.058 | T = 0.008 | | | |
| 1004552 | 222241 | 71288391 | C/T | C = 0.605 | C = 0.623 | 0.582 | 1.08 | T |
| | | | | T = 0.395 | T = 0.377 | | | |
| 4307892 | 222458 | 71288608 | G/A | G = 0.845 | G = 0.92 | 0.000389 | 2.10 | A |
| | | | | A = 0.155 | A = 0.080 | | | |
| 12431973 | 222735 | 71288885 | T/G | T = | T = 1.000 | | | |
| | | | | G = | G = 0 | | | |
| 12431975 | 222749 | 71288899 | A/G | A = 0.808 | A = 0.747 | 0.0315 | 0.70 | A |
| | | | | G = 0.192 | G = 0.253 | | | |
| 767874 | 223187 | 71289337 | T/C | T = 1.000 | T = 1.000 | 0.942 | 16.74 | C |
| | | | | C = 0.000 | C = 0.000 | | | |
| 11628597 | 226626 | 71292776 | G/C | G = 0.799 | G = 0.824 | 0.326 | 1.18 | C |
| | | | | C = 0.201 | C = 0.176 | | | |
| 10129371 | 226865 | 71293015 | A/G | A = 0.59 | A = 0.532 | 0.0655 | 0.79 | A |
| | | | | G = 0.410 | G = 0.468 | | | |
| 4903069 | 227548 | 71293698 | A/C | A = 0.928 | A = 0.932 | 0.828 | 1.06 | C |
| | | | | C = 0.072 | C = 0.068 | | | |
| 4903070 | 227690 | 71293840 | A/G | A = 0.572 | A = 0.576 | 0.883 | 1.02 | G |
| | | | | G = 0.428 | G = 0.424 | | | |
| 4903071 | 227815 | 71293965 | G/T | G = 0.692 | G = 0.752 | 0.0364 | 1.35 | T |
| | | | | T = 0.308 | T = 0.248 | | | |
| 11622224 | 228456 | 71294606 | T/C | T = 0.661 | T = 0.67 | 0.754 | 1.04 | C |
| | | | | C = 0.339 | C = 0.330 | | | |
| 10459455 | 229025 | 71295175 | G/A | G = 0.709 | G = 0.705 | 0.879 | 0.98 | G |
| | | | | A = 0.291 | A = 0.295 | | | |
| 9323577 | 229085 | 71295235 | C/T | C = 0.654 | C = 0.599 | 0.169 | 0.79 | C |
| | | | | T = 0.346 | T = 0.401 | | | |
| 10624648 | 231757 | 71297907 | TTG/— | TTG = 0.998 | TTG = 0.985 | 0.21 | 0.15 | TTG |
| | | | | — = 0.002 | — = 0.015 | | | |
| 3058953 | 231762 | 71297912 | GTT/— | GTT = 0.995 | GTT = 0.976 | 0.14 | 0.22 | GTT |
| | | | | — = 0.005 | — = 0.024 | | | |
| 11158981 | 232727 | 71298877 | T/G | T = 0.716 | T = 0.689 | 0.345 | 0.88 | T |
| | | | | G = 0.284 | G = 0.311 | | | |
| 11158982 | 232955 | 71299105 | T/G | T = 0.399 | T = 0.421 | 0.496 | 1.09 | G |
| | | | | G = 0.601 | G = 0.579 | | | |
| 11158983 | 233302 | 71299452 | G/T | G = 0.734 | G = 0.71 | 0.407 | 0.89 | G |
| | | | | T = 0.266 | T = 0.290 | | | |
| 1860750 | 233629 | 71299779 | T/A | T = 0.554 | T = 0.591 | 0.309 | 1.16 | A |
| | | | | A = 0.446 | A = 0.409 | | | |
| 1860749 | 233670 | 71299820 | G/A | G = 0.347 | G = 0.477 | 9.51E−05 | 1.71 | A |
| | | | | A = 0.653 | A = 0.523 | | | |
| 1860748 | 233702 | 71299852 | A/C | A = 0.108 | A = 0.184 | 0.00235 | 1.86 | C |
| | | | | C = 0.892 | C = 0.816 | | | |
| 1860747 | 233715 | 71299865 | A/G | A = 0.998 | A = 0.955 | 0.0811 | 0.05 | A |
| | | | | G = 0.002 | G = 0.045 | | | |
| 11419679 | 235827 | 71301977 | —/A | — = 0.597 | — = 0.567 | 0.36 | 0.88 | — |
| | | | | A = 0.403 | A = 0.433 | | | |
| 8011777 | 236951 | 71303101 | T/C | T = 0.993 | T = | | | |
| | | | | C = 0.007 | C = | | | |
| 6574099 | 237157 | 71303307 | A/G | A = 0.368 | A = 0.363 | 0.879 | 0.98 | A |
| | | | | G = 0.632 | G = 0.637 | | | |
| 8010957 | 237317 | 71303467 | A/G | A = 0.09 | A = 0.199 | 0.000106 | 2.53 | G |
| | | | | G = 0.910 | G = 0.801 | | | |
| 11626895 | 237363 | 71303513 | G/A | G = 0.744 | G = 0.73 | 0.634 | 0.93 | G |
| | | | | A = 0.256 | A = 0.270 | | | |
| 10151431 | 238866 | 71305016 | T/G | T = 0 | T = 0 | 1 | | |
| | | | | G = 1.000 | G = 1.000 | | | |
| 4903072 | 239789 | 71305939 | C/A | C = 0.451 | C = 0.462 | 0.747 | 1.04 | A |
| | | | | A = 0.549 | A = 0.538 | | | |
| 4589489 | 240170 | 71306320 | T/C | T = 0.001 | T = 0.001 | 0.979 | 1.61 | C |
| | | | | C = 0.999 | C = 0.999 | | | |
| 9671327 | 240430 | 71306580 | G/T | G = 0.828 | G = 0.842 | 0.558 | 1.11 | T |
| | | | | T = 0.172 | T = 0.158 | | | |
| 4899446 | 240454 | 71306604 | C/T | C = 0.552 | C = 0.574 | 0.497 | 1.09 | T |
| | | | | T = 0.448 | T = 0.426 | | | |
| 10144501 | 240688 | 71306838 | T/A | T = 0.001 | T = 0.001 | 0.976 | 0.57 | T |
| | | | | A = 0.999 | A = 0.999 | | | |
| 5809594 | 241414 | 71307564 | TG/— | TG = 0.55 | TG = 0.533 | 0.593 | 0.93 | TG |
| | | | | — = 0.450 | — = 0.467 | | | |
| 720317 | 241424 | 71307574 | A/G | A = 0.3 | A = 0.322 | 0.582 | 1.11 | G |
| | | | | G = 0.700 | G = 0.678 | | | |
| 8014316 | 241741 | 71307891 | G/A | G = 0.001 | G = 0.001 | 0.933 | 2.97 | A |
| | | | | A = 0.999 | A = 0.999 | | | |
| 11158984 | 241768 | 71307918 | T/C | T = 0.438 | T = 0.453 | 0.633 | 1.06 | C |
| | | | | C = 0.562 | C = 0.547 | | | |
| 6420906 | 242360 | 71308510 | C/T | C = 0.007 | C = 0.048 | 0.00105 | 7.74 | T |
| | | | | T = 0.993 | T = 0.952 | | | |
| 8004055 | 242957 | 71309107 | A/T | A = 1.000 | A = 1.000 | 0.986 | 2.23 | T |
| | | | | T = 0.000 | T = 0.000 | | | |
| 12434800 | 243355 | 71309505 | A/G | A = 0.132 | A = 0.069 | 0.00428 | 0.49 | A |
| | | | | G = 0.868 | G = 0.931 | | | |
| 4243644 | 243596 | 71309746 | T/A | T = 0.609 | T = 0.575 | 0.308 | 0.87 | T |
| | | | | A = 0.391 | A = 0.425 | | | |
| 4899447 | 243648 | 71309798 | A/G | A = 0.815 | A = 0.816 | 0.981 | 1.00 | G |
| | | | | G = 0.185 | G = 0.184 | | | |
| 4903073 | 243664 | 71309814 | G/A | G = 0.176 | G = 0.176 | 0.999 | 1.00 | A |
| | | | | A = 0.824 | A = 0.824 | | | |
| 11848634 | 243783 | 71309933 | T/C | T = 0.001 | T = 0 | 0.927 | 0.00 | T |
| | | | | C = 0.999 | C = 1.000 | | | |
| 4899448 | 243819 | 71309969 | T/C | T = 0.792 | T = 0.769 | 0.396 | 0.87 | T |
| | | | | C = 0.208 | C = 0.231 | | | |
| 10131564 | 245765 | 71311915 | A/G | A = 0.72 | A = 0.708 | 0.694 | 0.95 | A |
| | | | | G = 0.280 | G = 0.292 | | | |
| 10134150 | 246089 | 71312239 | T/G | T = 0.713 | T = 0.685 | 0.354 | 0.88 | T |
| | | | | G = 0.287 | G = 0.315 | | | |
| 763388 | 246457 | 71312607 | A/G | A = 0.71 | A = 0.694 | 0.592 | 0.93 | A |
| | | | | G = 0.290 | G = 0.306 | | | |
| 11845938 | 246485 | 71312635 | A/G | A = 0 | A = 0.001 | 0.975 | | G |
| | | | | G = 1.000 | G = 0.999 | | | |
| 11845946 | 246536 | 71312686 | G/T | G = 0.999 | G = 1.000 | 0.905 | 3.40 | T |
| | | | | T = 0.001 | T = 0.000 | | | |
| 11845915 | 246586 | 71312736 | T/C | T = 0.004 | T = 0.002 | 0.837 | 0.49 | T |
| | | | | C = 0.996 | C = 0.998 | | | |
| 10137463 | 247356 | 71313506 | A/C | A = 0.998 | A = 0.998 | 0.989 | 1.05 | C |
| | | | | C = 0.002 | C = 0.002 | | | |
| 6574100 | 247573 | 71313723 | T/A | T = 0.409 | T = 0.418 | 0.767 | 1.04 | A |
| | | | | A = 0.591 | A = 0.582 | | | |
| 7140344 | 248173 | 71314323 | A/G | A = 0 | A = 0.001 | 0.96 | | G |
| | | | | G = 1.000 | G = 0.999 | | | |
| 10646889 | 248915 | 71315065 | AA/— | AA = | AA = 0.604 | | | |
| | | | | — = | — = 0.396 | | | |
| 7146287 | 249344 | 71315494 | C/T | C = 1.000 | C = 1.000 | 0.975 | 0.00 | C |
| | | | | T = 0 | T = 0.000 | | | |
| 5809595 | 250575 | 71316725 | A/— | A = 0.406 | A = 0.384 | 0.505 | 0.91 | A |
| | | | | — = 0.594 | — = 0.616 | | | |
| 1035099 | 250701 | 71316851 | T/A | T = 0.447 | T = 0.457 | 0.781 | 1.04 | A |
| | | | | A = 0.553 | A = 0.543 | | | |
| 7159877 | 251260 | 71317410 | T/G | T = 0.291 | T = 0.329 | 0.211 | 1.19 | G |
| | | | | G = 0.709 | G = 0.671 | | | |
| 7142579 | 251858 | 71318008 | G/A | G = 0.037 | G = 0.075 | 0.0275 | 2.10 | A |
| | | | | A = 0.963 | A = 0.925 | | | |
| 12431762 | 251981 | 71318131 | G/A | G = 0.867 | G = 0.871 | 0.855 | 1.04 | A |
| | | | | A = 0.133 | A = 0.129 | | | |
| 12431764 | 251990 | 71318140 | G/A | G = 0.611 | G = 0.595 | 0.612 | 0.94 | G |
| | | | | A = 0.389 | A = 0.405 | | | |
| 10647999 | 252326 | 71318476 | AGAAAG | AGAAAGAA | AGAAAGA | 0.936 | 0.99 | AGAAAGAAAG |
| | | | AAAG/— | AG = 0.492 | AAG = 0.489 | | | (SEQ ID NO: |
| | | | (SEQ ID | (SEQ ID NO: | (SEQ ID NO: | | | 4936) |
| | | | NO: 4936) | 4936) | 4936) | | | |
| | | | | — = 0.508 | — = 0.511 | | | |
| 6574101 | 252727 | 71318877 | C/T | C = 0.737 | C = 0.707 | 0.313 | 0.86 | C |
| | | | | T = 0.263 | T = 0.293 | | | |
| 6574102 | 252946 | 71319096 | T/C | T = 0.747 | T = 0.762 | 0.585 | 1.09 | C |
| | | | | C = 0.253 | C = 0.238 | | | |
| 1861162 | 254199 | 71320349 | T/C | T = 0.513 | T = 0.523 | 0.787 | 1.04 | C |
| | | | | C = 0.487 | C = 0.477 | | | |
| 12432972 | 254985 | 71321135 | T/C | T = 1.000 | T = 0.999 | 0.953 | 0.58 | T |
| | | | | C = 0.000 | C = 0.001 | | | |
| 10146134 | 255324 | 71321474 | A/T | A = 0.69 | A = 0.706 | 0.598 | 1.08 | T |
| | | | | T = 0.310 | T = 0.294 | | | |
| 10146616 | 255686 | 71321836 | A/C | A = 0.858 | A = 0.84 | 0.448 | 0.87 | A |
| | | | | C = 0.142 | C = 0.160 | | | |
| 10146769 | 255718 | 71321868 | A/G | A = 0.052 | A = 0.081 | 0.0933 | 1.62 | G |
| | | | | G = 0.948 | G = 0.919 | | | |
| 8018936 | 256321 | 71322471 | G/A | G = | G = 0.995 | | | |
| | | | | A = | A = 0.005 | | | |
| 10483849 | 257562 | 71323712 | G/A | G = 0.302 | G = 0.291 | 0.72 | 0.95 | G |
| | | | | A = 0.698 | A = 0.709 | | | |
| 2160137 | 258843 | 71324993 | T/C | T = 0.842 | T = 0.811 | 0.242 | 0.81 | T |
| | | | | C = 0.158 | C = 0.189 | | | |
| 2192595 | 258880 | 71325030 | C/A | C = 0.861 | C = 0.834 | 0.422 | 0.81 | C |
| | | | | A = 0.139 | A = 0.166 | | | |
| 2192594 | 259060 | 71325210 | A/G | A = 0.374 | A = 0.383 | 0.789 | 1.04 | G |
| | | | | G = 0.626 | G = 0.617 | | | |
| 11271695 | 261590 | 71327740 | TCCATA | TCCATATG | TCCATAT | 0.947 | 0.79 | TCCATATGG |
| | | | TGGCTG | GCTGAGGA | GGCTGAG | | | CTGAGGAGG |
| | | | AGGAG | GG = 0.999 | GAGG = 0.998 | | | (SEQ ID NO: |
| | | | G/— | (SEQ ID NO: | (SEQ ID NO: | | | 4937) |
| | | | (SEQ ID | 4937) | 4937) | | | |
| | | | NO: 4937) | — = 0.001 | — = 0.002 | | | |
| 10747301 | 262088 | 71328238 | G/C | G = 0.995 | G = 0.986 | 0.42 | 0.37 | G |
| | | | | C = 0.005 | C = 0.014 | | | |
| 12050363 | 262345 | 71328495 | C/G | C = 0.993 | C = 0.975 | 0.232 | 0.28 | C |
| | | | | G = 0.007 | G = 0.025 | | | |
| 12050368 | 262368 | 71328518 | G/A | G = 0.947 | G = 0.945 | 0.929 | 0.97 | G |
| | | | | A = 0.053 | A = 0.055 | | | |
| 12432949 | 263858 | 71330008 | A/G | A = 0.606 | A = 0.601 | 0.872 | 0.98 | A |
| | | | | G = 0.394 | G = 0.399 | | | |
| 7401375 | 263995 | 71330145 | T/A | T = 1.000 | T = 0.996 | 0.586 | 0.02 | T |
| | | | | A = 0.000 | A = 0.004 | | | |
| 8019682 | 265195 | 71331345 | G/A | G = 0.848 | G = 0.812 | 0.293 | 0.77 | G |
| | | | | A = 0.152 | A = 0.188 | | | |
| 8021248 | 265620 | 71331770 | T/C | T = 0.597 | T = 0.624 | 0.426 | 1.12 | C |
| | | | | C = 0.403 | C = 0.376 | | | |
| 11158985 | 265833 | 71331983 | T/A | T = 0.242 | T = 0.24 | 0.962 | 0.99 | T |
| | | | | A = 0.758 | A = 0.760 | | | |
| 10483850 | 266387 | 71332537 | A/G | A = 0.823 | A = 0.86 | 0.142 | 1.32 | G |
| | | | | G = 0.177 | G = 0.140 | | | |
| 8010875 | 267130 | 71333280 | A/G | A = 0.0630 | A = 0.11 | 0.0193 | 1.84 | G |
| | | | | G = 0.937 | G = 0.890 | | | |
| 11626844 | 267468 | 71333618 | T/C | T = 0.424 | T = 0.416 | 0.802 | 0.97 | T |
| | | | | C = 0.576 | C = 0.584 | | | |
| 982972 | 267606 | 71333756 | A/G | A = 0.56 | A = 0.542 | 0.575 | 0.93 | A |
| | | | | G = 0.440 | G = 0.458 | | | |
| 7161198 | 268858 | 71335008 | T/C | T = 0.563 | T = 0.573 | 0.75 | 1.05 | C |
| | | | | C = 0.437 | C = 0.427 | | | |
| 7160347 | 269193 | 71335343 | G/A | G = 0.551 | G = 0.553 | 0.966 | 1.01 | A |
| | | | | A = 0.449 | A = 0.447 | | | |
| 8007139 | 269991 | 71336141 | A/G | A = 0.735 | A = 0.735 | 0.992 | 1.00 | A |
| | | | | G = 0.265 | G = 0.265 | | | |
| 8014483 | 271840 | 71337990 | C/T | C = 0.757 | C = 0.762 | 0.867 | 1.03 | T |
| | | | | T = 0.243 | T = 0.238 | | | |
| 12437072 | 272592 | 71338742 | T/C | T = 0.887 | T = 0.905 | 0.414 | 1.21 | C |
| | | | | C = 0.113 | C = 0.095 | | | |
| 8004767 | 273399 | 71339549 | C/G | C = 0.865 | C = 0.803 | 0.0203 | 0.64 | C |
| | | | | G = 0.135 | G = 0.197 | | | |
| 12588830 | 273504 | 71339654 | A/G | A = 0.304 | A = 0.305 | 0.986 | 1.00 | G |
| | | | | G = 0.696 | G = 0.695 | | | |
| 7158449 | 274050 | 71340200 | T/C | T = 0.001 | T = 0.001 | 0.944 | 0.23 | T |
| | | | | C = 0.999 | C = 0.999 | | | |
| 6574103 | 274182 | 71340332 | C/T | C = 0.033 | C = 0.028 | 0.682 | 0.83 | C |
| | | | | T = 0.967 | T = 0.972 | | | |
| 8020478 | 274275 | 71340425 | C/T | C = 0.999 | C = 0.998 | 0.91 | 0.59 | C |
| | | | | T = 0.001 | T = 0.002 | | | |
| 4903077 | 276293 | 71342443 | C/T | C = 0.971 | C = 0.973 | 0.878 | 1.09 | T |
| | | | | T = 0.029 | T = 0.027 | | | |
| 8012359 | 276645 | 71342795 | G/A | G = 0.292 | G = 0.285 | 0.803 | 0.96 | G |
| | | | | A = 0.708 | A = 0.715 | | | |
| 4899449 | 277166 | 71343316 | A/G | A = 0.927 | A = 1.000 | 2.12E−06 | 113.75 | G |
| | | | | G = 0.073 | G = 0.000 | | | |
| 10690289 | 278290 | 71344440 | AAG/— | AAG = 0.977 | AAG = 0.992 | 0.239 | 2.65 | — |
| | | | | — = 0.023 | — = 0.008 | | | |
| 6574104 | 278646 | 71344796 | G/T | G = 0.959 | G = 0.975 | 0.328 | 1.65 | T |
| | | | | T = 0.041 | T = 0.025 | | | |
| 2110552 | 279022 | 71345172 | A/G | A = 0.747 | A = 0.761 | 0.638 | 1.08 | G |
| | | | | G = 0.253 | G = 0.239 | | | |
| 8008256 | 279073 | 71345223 | G/A | G = 0.497 | G = 0.474 | 0.48 | 0.91 | G |
| | | | | A = 0.503 | A = 0.526 | | | |
| 759283 | 280198 | 71346348 | G/T | G = 0.645 | G = 0.64 | 0.88 | 0.98 | G |
| | | | | T = 0.355 | T = 0.360 | | | |
| 4999177 | 281100 | 71347250 | A/G | A = 0.001 | A = 0.003 | 0.853 | 37.48 | G |
| | | | | G = 0.999 | G = 0.997 | | | |
| 2333006 | 281145 | 71347295 | C/G | C = 0.002 | C = 0.007 | 0.719 | 4.08 | G |
| | | | | G = 0.998 | G = 0.993 | | | |
| 8006178 | 282655 | 71348805 | A/C | A = 0.802 | A = 0.787 | 0.583 | 0.91 | A |
| | | | | C = 0.198 | C = 0.213 | | | |
| 11158986 | 282836 | 71348986 | A/C | A = 0.0630 | A = 0.054 | 0.667 | 0.85 | A |
| | | | | C = 0.937 | C = 0.946 | | | |
| 7150625 | 283537 | 71349687 | C/T | C = 0.999 | C = 0.997 | 0.849 | 0.55 | C |
| | | | | T = 0.001 | T = 0.003 | | | |
| 8007768 | 283976 | 71350126 | T/G | T = 0.0660 | T = 0.078 | 0.659 | 1.21 | G |
| | | | | G = 0.934 | G = 0.922 | | | |
| 8007058 | 284149 | 71350299 | A/G | A = 0.592 | A = | | | |
| | | | | G = 0.408 | G = | | | |
| DPF3-AA | 284742 | 71350892 | C/A | C = 0.86 | C = 0.88 | 0.381 | 1.20 | A |
| | | | | A = 0.140 | A = 0.120 | | | |
| 2215901 | 284840 | 71350990 | C/T | C = 0.966 | C = 0.991 | 0.163 | 3.66 | T |
| | | | | T = 0.034 | T = 0.009 | | | |
| 3814866 | 284912 | 71351062 | T/G | T = 0.774 | T = 0.779 | 0.854 | 1.03 | G |
| | | | | G = 0.226 | G = 0.221 | | | |
| 3814864 | 285091 | 71351241 | T/C | T = 0.464 | T = 0.44 | 0.494 | 0.91 | T |
| | | | | C = 0.536 | C = 0.560 | | | |
| 10605948 | 250079-250082 | 71316232 | CCCC/— | CCCC = 0.649 | CCCC = 0.737 | 0.00471 | 1.51 | — |
| | | | | — = 0.351 | — = 0.263 | | | |

FIG. 1F shows proximal SNPs in and around the DPF3 region for females. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1F can be determined by consulting Table 44. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Wed at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 17

### LOC145197 Proximal SNPs

It has been discovered that a polymorphic variation (rs1054745) in the LOC145197 gene is associated with the occurrence of breast cancer (see Examples 1 and 2). Subsequently, SNPs proximal to the incident SNP (rs1054745) were identified and allelotyped in breast cancer sample sets and control sample sets as described in Examples 1 and 2. Approximately 106 allelic variants located within the LOC145197 gene were identified and allelotyped. The polymorphic variants are set forth in Table 45. The chromosome position provided in column four of Table 45 is based on Genome “Build 34” of NCBI's GenBank.

TABLE 45

| | Position | | | | | |
|---|---|---|---|---|---|---|
| | in | | Chromo- | Allele | | De- |
| dbSNP | SEQ ID | Chromo- | some | Vari- | Genome | duced |
| rs# | NO: 7 | some | Position | ants | Letter | Iupac |
| 2895907 | 225 | 14 | 99854375 | a/c | a | M |
| 2401019 | 505 | 14 | 99854655 | g/a | g | R |
| 2401020 | 5170 | 14 | 99859320 | t/c | c | Y |
| 2401021 | 5183 | 14 | 99859333 | t/c | c | Y |
| 1999611 | 6839 | 14 | 99860989 | t/c | g | R |
| 1999610 | 7016 | 14 | 99861166 | c/t | a | R |
| 1999609 | 7237 | 14 | 99861387 | c/t | g | R |
| 2275016 | 8190 | 14 | 99862340 | c/t | g | R |
| 2275015 | 8357 | 14 | 99862507 | c/t | a | R |
| 3818772 | 8577 | 14 | 99862727 | c/g | c | S |
| 10149458 | 15999 | 14 | 99870149 | c/t | c | Y |
| 7142383 | 17722 | 14 | 99871872 | c/t | c | Y |
| 11343023 | 18543 | 14 | 99872693 | t/— | t | N |
| 11160641 | 21639 | 14 | 99875789 | c/a | c | M |
| 12586334 | 24753 | 14 | 99878903 | c/a | a | M |
| 10142208 | 26289 | 14 | 99880439 | g/a | g | R |
| 7493230 | 27120 | 14 | 99881270 | c/a | a | M |
| 5811036 | 28737 | 14 | 99882887 | a/— | a | N |
| 10151926 | 29562 | 14 | 99883712 | t/a | a | W |
| 12435354 | 30078 | 14 | 99884228 | t/c | t | Y |
| 7151007 | 30974 | 14 | 99885124 | c/t | c | Y |
| 11160642 | 31545 | 14 | 99885695 | t/c | c | Y |
| 11160643 | 31779 | 14 | 99885929 | t/a | t | W |
| 11626687 | 34062 | 14 | 99888212 | t/c | c | Y |
| 4906112 | 34831 | 14 | 99888981 | t/g | t | K |
| 8016071 | 34906 | 14 | 99889056 | g/c | g | S |
| 11623698 | 34934 | 14 | 99889084 | t/g | t | K |
| 7401628 | 34938 | 14 | 99889088 | t/g | t | K |
| 8015995 | 35131 | 14 | 99889281 | g/a | a | R |
| 10873526 | 35511 | 14 | 99889661 | t/c | t | Y |
| 11160644 | 35708 | 14 | 99889858 | a/g | g | R |
| 10134252 | 36071 | 14 | 99890221 | g/c | g | S |
| 1959030 | 36824 | 14 | 99890974 | a/t | t | W |
| 9707396 | 36959 | 14 | 99891109 | t/g | g | K |
| 2401022 | 37335 | 14 | 99891485 | a/t | t | W |
| 4622448 | 37343 | 14 | 99891493 | a/g | g | R |
| 10141019 | 38507 | 14 | 99892657 | c/t | c | Y |
| 10141096 | 38554 | 14 | 99892704 | a/c | c | M |
| 11621063 | 38624 | 14 | 99892774 | c/t | t | Y |
| 12434632 | 38690 | 14 | 99892840 | t/c | c | Y |
| 12100901 | 38744 | 14 | 99892894 | t/c | c | Y |
| 12434655 | 38756 | 14 | 99892906 | t/c | c | Y |
| 9944103 | 39525 | 14 | 99893675 | g/a | g | R |
| 7153189 | 40682 | 14 | 99894832 | g/a | a | R |
| 7158860 | 41181 | 14 | 99895331 | a/g | g | R |
| 12437080 | 41414 | 14 | 99895564 | g/c | g | S |
| 11851768 | 41657 | 14 | 99895807 | g/a | a | R |
| 12431943 | 42821 | 14 | 99896971 | g/a | a | R |
| 4309341 | 45475 | 14 | 99899625 | g/a | g | R |
| 4329858 | 45611 | 14 | 99899761 | g/c | g | S |
| 10690268 | 45763 | 14 | 99899913 | aaa/— | a | N |
| 2401023 | 46038 | 14 | 99900188 | t/c | c | Y |
| 1054745 | 46344 | 14 | 99900494 | g/a | a | R |
| 8019453 | 46669 | 14 | 99900819 | t/c | c | Y |
| 12431510 | 46926 | 14 | 99901076 | g/c | g | S |
| 7141067 | 47069 | 14 | 99901219 | t/c | c | Y |
| 12431547 | 47293 | 14 | 99901443 | g/a | a | R |
| 7142316 | 47602 | 14 | 99901752 | g/a | g | R |
| 1959029 | 49821 | 14 | 99903971 | g/a | c | Y |
| 1959028 | 49875 | 14 | 99904025 | t/c | a | R |
| 11376292 | 51178 | 14 | 99905328 | —/a | g | N |
| 11376755 | 51180 | 14 | 99905330 | a/— | a | N |
| 7145420 | 51772 | 14 | 99905922 | a/g | g | R |
| 8013347 | 51912 | 14 | 99906062 | g/t | t | K |
| 2144070 | 52252 | 14 | 99906402 | t/c | t | Y |
| 11850135 | 52294 | 14 | 99906444 | g/a | g | R |
| 11850138 | 52317 | 14 | 99906467 | a/g | g | R |
| 1955617 | 54548 | 14 | 99908698 | g/a | g | R |
| 11621899 | 54889 | 14 | 99909039 | t/c | c | Y |
| 4900508 | 55377 | 14 | 99909527 | c/a | a | M |
| 4906116 | 55498 | 14 | 99909648 | t/c | c | Y |
| 4906117 | 55622 | 14 | 99909772 | c/t | c | Y |
| 2235972 | 55799 | 14 | 99909949 | c/t | c | Y |
| 2235973 | 55995 | 14 | 99910145 | t/c | c | Y |
| 2235974 | 56042 | 14 | 99910192 | c/a | a | M |
| 2235975 | 56342 | 14 | 99910492 | g/a | g | R |
| 7149416 | 57781 | 14 | 99911931 | t/c | c | Y |
| 11850809 | 57983 | 14 | 99912133 | a/c | c | M |
| 10140476 | 58906 | 14 | 99913056 | t/c | t | Y |
| 12437309 | 59257 | 14 | 99913407 | c/t | c | Y |
| 6575852 | 60926 | 14 | 99915076 | t/g | t | K |
| 4906118 | 61462 | 14 | 99915612 | a/g | a | R |
| 11428825 | 62241 | 14 | 99916391 | —/t | t | N |
| 11450744 | 62541 | 14 | 99916691 | —/t | a | N |
| 1999612 | 63600 | 14 | 99917750 | t/c | g | R |
| 1889368 | 63978 | 14 | 99918128 | g/a | t | Y |
| 1889367 | 63983 | 14 | 99918133 | g/c | c | S |
| 1889366 | 63999 | 14 | 99918149 | g/a | t | Y |
| 3077388 | 64042 | 14 | 99918192 | —/ct | t | N |
| 1889365 | 64276 | 14 | 99918426 | g/a | t | Y |
| 1955615 | 66308 | 14 | 99920458 | a/g | a | R |
| 1569807 | 67400 | 14 | 99921550 | t/c | c | Y |
| 2064491 | 69255 | 14 | 99923405 | c/t | c | Y |
| 2064492 | 69263 | 14 | 99923413 | t/c | t | Y |
| 761532 | 70965 | 14 | 99925115 | c/t | t | Y |
| 1955616 | 71540 | 14 | 99925690 | a/g | g | R |
| 2144065 | 74698 | 14 | 99928848 | g/a | g | R |
| 2144066 | 74746 | 14 | 99928896 | c/t | c | Y |
| 2895908 | 75221 | 14 | 99929371 | a/g | a | R |
| 2401043 | 91289 | 14 | 99945439 | t/a | a | W |
| 1007904 | 91796 | 14 | 99945946 | a/g | g | R |
| 3891113 | 93762 | 14 | 99947912 | c/g | g | S |
| 3891114 | 93780 | 14 | 99947930 | g/c | g | S |
| 730812 | 94242 | 14 | 99948392 | t/g | t | K |
| 1884068 | 94809 | 14 | 99948959 | a/g | g | R |
| 1884069 | 94816 | 14 | 99948966 | c/t | t | Y |

### Assay for Verifying and Allelotyping SNPs

The methods used to verify and allelotype the proximal SNPs of Table 45 are the same methods described in Examples 1 and 2 herein. The PCR primers and extend primers used in these assays are provided in Table 46 (SEQ ID NO: 4609-4820, respectively, in order of appearance) and Table 47 (SEQ ID NO: 4821-4926, respectively, in order of appearance), respectively.

TABLE 46

| | | |
|---|---|---|
| dbSNP rs# | Forward PCR primer | Reverse PCR primer |

| | | |
|---|---|---|
| 2895907 | ACGTTGGATGAGCGAGAACTTATCTCTACC | ACGTTGGATGATCTTCTGACTAGCTGGGAC |
| 2401019 | ACGTTGGATGGTGATGCCAGATACTTCCCG | ACGTTGGATGAGCACTGTGGGTCACTGTGG |
| 2401020 | ACGTTGGATGCATTGTGCCTCAGTTTCCTC | ACGTTGGATGTTATGGAGCCCTTTCCACAG |
| 2401021 | ACGTTGGATGAGCATTGTGCCTCAGTTTCC | ACGTTGGATGTTATGGAGCCCTTTCCACAG |
| 1999611 | ACGTTGGATGTGAAAGCCCTTGGCAGGAGA | ACGTTGGATGAGACACTGGACCTAACTGAG |
| 1999610 | ACGTTGGATGACCACCTCCCCACTATTCTG | ACGTTGGATGAACTCTGAGGTTCCAACCTG |
| 1999609 | ACGTTGGATGACAAAAGGACTCGGATCTGG | ACGTTGGATGAGCTCTAGGGGAATCCTCTC |
| 2275016 | ACGTTGGATGCTAGATCATTGGCCAGTGTG | ACGTTGGATGAAGAGGCATGCCGAAATGAG |
| 2275015 | ACGTTGGATGTGACCTGCACAGCCCGGGAGA | ACGTTGGATGCCAATGAAAGAGGAGATGCG |
| 3818772 | ACGTTGGATGGGTGGCTAGTAGGATGATTG | ACGTTGGATGGCATGGAACCCCACATGTAC |
| 10149458 | ACGTTGGATGTGATCAAGGTTCCATCTTAG | ACGTTGGATGTATCCACCATCGTTCCAAAC |
| 7142383 | ACGTTGGATGCCTGGCTCATTGAAGCATTC | ACGTTGGATGGAAGTTCACATTTCCAAGGG |
| 11343023 | ACGTTGGATGTTTCATGCTTTTGGGACCAG | ACGTTGGATGGATTTCCCTATTTAACTGGC |
| 11160641 | ACGTTGGATGACTTGAGCTCAGGAGTTTGA | ACGTTGGATGTACAGGTTTGTGCCACCATG |
| 12586334 | ACGTTGGATGCTATCCTTCTACCAAAGGAT | ACGTTGGATGTCAGAGTACTAGTCTTGAAC |
| 10142208 | ACGTTGGATGAACAATTCTTCTGACGCTGC | ACGTTGGATGGGCATATACTGTATGGTTCC |
| 7493230 | ACGTTGGATGATCCAGTCCTAGTTCCTTTC | ACGTTGGATGAGTCTCTTCTGGCTGTTTAC |
| 5811036 | ACGTTGGATGGGAGAGATCGCTGGCTTGAT | ACGTTGGATGTTGCAGTGAGCTGAGATTGC |
| 10151926 | ACGTTGGATGCCTCTGTGTTAAGTCTATTC | ACGTTGGATGTAGCACATAGCTAGATACTG |
| 12435354 | ACGTTGGATGACCTTCACTGAGAACACTGG | ACGTTGGATGACCTGGCCAATATGATGAGG |
| 7151007 | ACGTTGGATGTGACACTGAGCATCTTTTCC | ACGTTGGATGGAATAGACACTTCACCAGAG |
| 11160642 | ACGTTGGATGTAGTCCTAGCTACACGGGAG | ACGTTGGATGAGTGCAGTGGCACGATCTTG |
| 11160643 | ACGTTGGATGAAATGCTGGGATTACAGGCG | ACGTTGGATGCTGGGTATAAACCCAAAGGG |
| 11626687 | ACGTTGGATGGGGAATCACAGAAAGTGTCC | ACGTTGGATGCACACGTGGTCATAATGTGC |
| 4906112 | ACGTTGGATGCCGGCCCTTTCTCATTTTTG | ACGTTGGATGATGAACCCAAAACCTGGTTC |
| 8016071 | ACGTTGGATGCAGAGTGGCTGTCAATGAAA | ACGTTGGATGCCTGATCAATCTGTATAGAG |
| 11623698 | ACGTTGGATGAAGAACCAGGTTTTGGGTTC | ACGTTGGATGAAAAAAGACCAGAGTGGCTG |
| 7401628 | ACGTTGGATGAAGAACCAGGTTTTGGGTTC | ACGTTGGATGCAGAGTGGCTGTCAATGAAA |
| 8015995 | ACGTTGGATGACCATTGGGATCGTGTTCAG | ACGTTGGATGCCGCTTAAGTGTTCCTCTAG |
| 10873526 | ACGTTGGATGATCCATTCTCATCACATCCC | ACGTTGGATGACAAACACTACGTCCGTCTG |
| 11160644 | ACGTTGGATGGGACTCCTCAAAACTGTCAG | ACGTTGGATGGTCTCTCTCAGCTTCTCTTG |
| 10134252 | ACGTTGGATGTTTGAACCTGGGAGGTGGAG | ACGTTGGATGTGGAGTCTTGTTCTGTCACC |
| 1959030 | ACGTTGGATGGAGGCACGCATCCAATTCAG | ACGTTGGATGGTGTCAGAGTGAGACTCTGT |
| 9707396 | ACGTTGGATGGGGTTCAAGCAATTCTCCTG | ACGTTGGATGAAATTAGATGGGCGTGGTGG |
| 2401022 | ACGTTGGATGACACAGGACTGTTGCAGTGG | ACGTTGGATGAAGACTACAGAGACTCCAGC |
| 4622448 | ACGTTGGATGTACAGAGACTCCAGCATCTG | ACGTTGGATGTCAGATTTCCAGCCACACAG |
| 10141019 | ACGTTGGATGGTCCATGAGAGACTTCCTTC | ACGTTGGATGGGGAATATTCGTGTCTGGAG |
| 10141096 | ACGTTGGATGACACGGTGCAGAGGGTAAAC | ACGTTGGATGCAAAACCCAGCTCTGACCTC |
| 11621063 | ACGTTGGATGTTTACCCTCTGCACCGTGTG | ACGTTGGATGTGAGAAGCTAACAGCACAGG |
| 12434632 | ACGTTGGATGCTCCTATGGTTAATGTTGGG | ACGTTGGATGGTGCTGTTAGCTTCTCATAC |
| 12100901 | ACGTTGGATGAACTGGAAAATCCCTGCTGC | ACGTTGGATGTGTCAGCTACTCCACTCAAC |
| 12434655 | ACGTTGGATGGGGTCATTATCAAAAAACTGG | ACGTTGGATGCCAACATTAACCATAGGAGAC |
| 9944103 | ACGTTGGATGGCCCAAGGATTTGGGTAAAC | ACGTTGGATGAATGCCGAATCCCTCAAAAG |
| 7153189 | ACGTTGGATGCAAACTTCCAAGATAGACTG | ACGTTGGATGTGACTTGTATTCTCCAATTC |
| 7158860 | ACGTTGGATGGTAAGTTTCATCCTGACCTC | ACGTTGGATGCTCCTTACTGACCTAAGGAG |
| 12437080 | ACGTTGGATGTAAAGAGGGAGACTGGAGAG | ACGTTGGATGCCTTAGTTCAACCCTACCAC |
| 11851768 | ACGTTGGATGACTTTCCTCAGGCCTCTTTG | ACGTTGGATGAAAGGACGTGTATGCTCACC |
| 12431943 | ACGTTGGATGTTCGGGATCTCACAGTCTAG | ACGTTGGATGGGGCCGTACCTCCTTTTAAA |
| 4309341 | ACGTTGGATGAAAGTACTGGGATTACAGGC | ACGTTGGATGAATCTCTTCCTGGGTCTTCG |
| 4329858 | ACGTTGGATGATTCTCTTGCCTCAGTCTCC | ACGTTGGATGCCTGACCGACATGGAGAATC |
| 10690268 | ACGTTGGATGCAAGAGCGAGACTCCGTTAA | ACGTTGGATGTTGATGATGCAGTCTTCGTG |
| 2401023 | ACGTTGGATGGGGACCACTAGAGTCTAAAC | ACGTTGGATGTGTCTGAAGCACCAGGAATG |
| 1054745 | ACGTTGGATGGACTTTTAGGTCTGAGTTGG | ACGTTGGATGCTTCCTCTAGCAGTGTATTTC |
| 8019453 | ACGTTGGATGCCAGTCGTTTCAAAAGGAAC | ACGTTGGATGGCTTCAATAGTCTGTAACCC |
| 12431510 | ACGTTGGATGAAATTAGCTGGGCATGGTGG | ACGTTGGATGCCCAGGTTCAAGTGATTCTC |
| 7141067 | ACGTTGGATGTTCTAGATGGAGGCGAAGAG | ACGTTGGATGCAAAGTGCTGGCATTACAGG |
| 12431547 | ACGTTGGATGGCTAACTAACGGCTAATAGG | ACGTTGGATGAAGAAGCTCAATAGGCACTG |
| 7142316 | ACGTTGGATGAGTATTCCAGAGGCTTGTGG | ACGTTGGATGTGACATCTGCCCAGCAGTAC |
| 1959029 | ACGTTGGATGGCTAAGCTCTGCTTGAATTC | ACGTTGGATGCGAGTTGGATTATAGACTGC |
| 1959028 | ACGTTGGATGAGTCTATAATCCAACTCGCC | ACGTTGGATGTGACAGAATGAGACTCTGTC |
| 11376292 | ACGTTGGATGCTGTTTTTTCTGCTTTCTTC | ACGTTGGATGGGCTCCACAATTTTTAAGTG |
| 11376755 | ACGTTGGATGGGCTCCACAATTTTTAAGTG | ACGTTGGATGCTGTTTTTTCTGCTTTCTTC |
| 7145420 | ACGTTGGATGTGCCCTTGGTGAATTTCACG | ACGTTGGATGTACCTAGAAGTGGTAGGGAC |
| 8013347 | ACGTTGGATGGCCGAAGATGTTTTCTACTG | ACGTTGGATGAGCAGCTATCCAGGTTATTC |
| 2144070 | ACGTTGGATGGCCTGCTCTGTACCAGTAAC | ACGTTGGATGTGTCTCACCCAATTTGGCTC |
| 11850135 | ACGTTGGATGGTTTTAGCAGCTCAGAAGGG | ACGTTGGATGCCTCACACAGTTACTGGTAC |
| 11850138 | ACGTTGGATGCCTCACACAGTTACTGGTAC | ACGTTGGATGGTTTTAGCAGCTCAGAAGGG |
| 1955617 | ACGTTGGATGGAAATAGAAGCACTTTGGGT | ACGTTGGATGCTTTTATAATAAAAATCTCTC |
| 11621899 | ACGTTGGATGACTAGGTAAGGGAGTGAATG | ACGTTGGATGTTGGAAGATATTCCTGGCAG |
| 4900508 | ACGTTGGATGCTAAGGTCTTCCTGTCACTC | ACGTTGGATGCCTTTCTTCCCAATTGCATG |
| 4906116 | ACGTTGGATGAAAACCTGCGTGGTTGGATG | ACGTTGGATGCAGGCTGGAGAAGTCTAGAA |
| 4906117 | ACGTTGGATGGCTACATTGAAGAAGGACGG | ACGTTGGATGTGCACGTCTTTAAACACAGC |
| 2235972 | ACGTTGGATGTATCTTTTTTCCCCCATCAG | ACGTTGGATGGTAGGTTCAGGTGTCATTTG |
| 2235973 | ACGTTGGATGCTTCTCGAGGTCAAGGTTTC | ACGTTGGATGGATGGTCAGGTCATCTCATG |
| 2235974 | ACGTTGGATGCCTTGACCTCGAGAAGAAAG | ACGTTGGATGGTATGGCTGGGTAAAAATGC |
| 2235975 | ACGTTGGATGGGTTTTACTGCAAATGTGCG | ACGTTGGATGTAAGTGGTAGGAAATGATAC |
| 7149416 | ACGTTGGATGTTCTTCCCTGACTCCCTAAC | ACGTTGGATGTGGAAGGCATTTCAAGCAGG |
| 11850809 | ACGTTGGATGCAGGGATCTACTCCTTGGTC | ACGTTGGATGGAAGATGCAAGGTCAGCAGG |
| 10140476 | ACGTTGGATGTTTCTAGAAATGCGAGCTGG | ACGTTGGATGGACCCATTTTCCAAATGTCAG |
| 12437309 | ACGTTGGATGCTGTCGAAGTATTGAGCACC | ACGTTGGATGCTGAAGGACCGAACACAATC |
| 6575852 | ACGTTGGATGACCTGGGTGCTGTGCTGCGG | ACGTTGGATGACGTGGGAACCGGGCTGTC |
| 4906118 | ACGTTGGATGCCTCTGAGGTTGCAAATCTG | ACGTTGGATGTGGCGCCCATTTGGCTTTTT |
| 11428825 | ACGTTGGATGAGAGCCCGTCTTTCTCCACA | ACGTTGGATGCCTTCAACCCATCTGGAAAG |
| 11450744 | ACGTTGGATGAAACCCCAGATATTCCCTTG | ACGTTGGATGTGAGATAAAAGACACACAGG |
| 1999612 | ACGTTGGATGACGTTGGAATGCCAGTTTGC | ACGTTGGATGTCCTGGGAATGGGCTTCTAC |
| 1889368 | ACGTTGGATGACCTCTAGAATTTCCTGGCC | ACGTTGGATGCAAGGACAAGTGTCTTAGGG |
| 1889367 | ACGTTGGATGACCTCTAGAATTTCCTGGCC | ACGTTGGATGTGTCGGCGACAAGGACAAGT |
| 1889366 | ACGTTGGATGCCCTGATATCTCTTACTCCC | ACGTTGGATGAGTGTGTTCACGGGCAATGG |
| 3077388 | ACGTTGGATGTGAACGCAGTGTGTTCACGG | ACGTTGGATGAAGACACTTGTCCTTGTCGC |
| 1889365 | ACGTTGGATGCATGCTGCAGCCAAACATTG | ACGTTGGATGTAGGCAAGGCACCATCTCAT |
| 1955615 | ACGTTGGATGCACTGCCCTGATGTCACACA | ACGTTGGATGTGCAGAGTGATAGGGTCAGG |
| 1569807 | ACGTTGGATGCTGCAGAGTTCAAAGATGCG | ACGTTGGATGTCAGGTCCTGGAGTTAAAAG |
| 2064491 | ACGTTGGATGAGTCACCAAAGCTAAGTGCC | ACGTTGGATGACACCGTCTCTGGGCTGAAG |
| 2064492 | ACGTTGGATGACACCGTCTCTGGGCTGAAG | ACGTTGGATGAAGCTAAGTGCCAAGATGCG |
| 761532 | ACGTTGGATGACCTTTAAGCCCAAGTTGAG | ACGTTGGATGACTGGCATGCAGTTGTTACC |
| 1955616 | ACGTTGGATGACCAACTTCCACTCAGAAGC | ACGTTGGATGCAGCATGGCTTCATCTTAAC |
| 2144065 | ACGTTGGATGAAATTAACCCTCGCAGAGGC | ACGTTGGATGGAGAAAGTTCCAGAAGAGGG |
| 2144066 | ACGTTGGATGCCTCTGCGAGGGTTAATTTC | ACGTTGGATGCAGAAACACCCAGAGTGATG |
| 2895908 | ACGTTGGATGAGAGATCAGAGAGAGAGAGC | ACGTTGGATGAAGGTGATTAGGTCAGGAGG |
| 2401043 | ACGTTGGATGGTTACACACTTTCTAGGCTC | ACGTTGGATGTGGGCCCAAACTATCTCTAG |
| 1007904 | ACGTTGGATGAGCGCAGTTGTTTGTCTCTC | ACGTTGGATGCCATGATGATGGAGCAACAG |
| 3891113 | ACGTTGGATGGGGTTTGGAGAAGAAAGGAG | ACGTTGGATGGTGACTCACTGTCTAGTCTG |
| 3891114 | ACGTTGGATGTCCTGTGACTCACTGTCTAG | ACGTTGGATGGAAAGGAGACTGGTTTGGG |
| 730812 | ACGTTGGATGATTGGTGAGGTCTGGCTATG | ACGTTGGATGCCTGTAATCCCAGCACTTTG |
| 1884068 | ACGTTGGATGCCACTGGGAAAAGGCACTT | ACGTTGGATGGTAGGGTTGTTGGGTCATTC |
| 1884069 | ACGTTGGATGCTTTCAGAATAAGGGCCAAC | ACGTTGGATGCTCTCAGACAGCGTTTTAAG |

TABLE 47

| dbSNP rs# | Extend Primer | Term Mix |
|---|---|---|
| 2895907 | ATCTCTACCAAACAATACAAAAAT | ACT |
| 2401019 | TGCGCAAAGTGCCAGAGCCC | ACG |
| 2401020 | TTCCGTGAATGGGAATG | ACT |
| 2401021 | CCTCAGTTTCCTCTTCC | ACT |
| 1999611 | AGGAGAGCAGCACTCAC | ACT |
| 1999610 | CCCCACTATTCTGATAACC | ACG |
| 1999609 | CAGAGTCCCCATGTGCACGC | ACG |
| 2275016 | GCCAGTGTGGTTGCAATT | ACG |
| 2275015 | AGCCGCTGCCCCAGGTG | ACG |
| 3818772 | TGCTTTGGTGCTCTCTT | ACT |
| 10149458 | CCAAATTTTGGTTGCTTCAAGG | ACG |
| 7142383 | TATAGACATTATCTGACAACACT | ACG |
| 11343023 | GGAGAATCTGCCCAGCTCAT | ACT |
| 11160641 | GCCTGGGTAACATGGTGAAA | CGT |
| 12586334 | AAAAACTTAAGATACATAGGGAC | CGT |
| 10142208 | CTTCTGACGCTGCTAACTAAC | ACG |
| 7493230 | AGTCCTAGTTCCTTTCTGTTTA | CGT |
| 5811036 | TTCCCAACTCTTTTTTTTTTTTTTT | ACT |
| 10151926 | TAGAGAGGATATAGAAAAGGAG | CGT |
| 12435354 | CTGAGAACACTGGTTGAGAGT | ACT |
| 7151007 | TTTTCCTGCCCTTATTTGTCAT | ACG |
| 11160642 | GAACCCAGGAGGCAGAGC | ACT |
| 11160643 | CCAGCCCACATGTGTCTTTAT | CGT |
| 11626687 | CACAGAAAGTGTCCTTTATAAAC | ACT |
| 4906112 | CCCTTTCTCATTTTTGATACTG | ACT |
| 8016071 | AGAGAAAACAAATGAACCCAAAA | ACT |
| 11623698 | GGGTTCATTTGTTTTCTCTATTG | ACT |
| 7401628 | TCATTTGTTTTCTCTATTGTCTG | ACT |
| 8015995 | CAGCTGAATGAAGACACTACA | ACG |
| 10873526 | CCATCAACATGGCTTATCACC | ACT |
| 11160644 | TCAGGGTCCTCAAAAATAAGG | ACT |
| 10134252 | GAGGTGGAGGTTGCAGTGA | ACT |
| 1959030 | ATTCAGCTGATGATCCC | CGT |
| 9707396 | TCTCCTGACTCAGTCTCCC | ACT |
| 2401022 | GGTCAGGCTCTTCAGCG | CGT |
| 4622448 | CCTGGTCTCTTCTCGCTGAA | ACT |
| 10141019 | CTGTCCTGCAAGCAGGGAA | ACG |
| 10141096 | GGTAAACCCAGGCTCACAG | ACT |
| 11621063 | GACAGGTGCTCCGCCAGA | ACG |
| 12434632 | TGTTGGGGTTGAGTGGAGTA | ACT |
| 12100901 | CCTGCTGCGGAGACTATTCA | ACT |
| 12434655 | AACTGGAAAATCCCTGCTGC | ACT |
| 9944103 | GGTAAACACACTCGCAACAG | ACG |
| 7153189 | TAGACTGCCTTTGTATATCTTTT | ACG |
| 7158860 | GCCAACAATGGCTACATCTTTT | ACT |
| 12437080 | GGAATTCTGTAATGTGGGCTG | ACT |
| 11851768 | GGTGCTACCTCTTTAGCCAC | ACG |
| 12431943 | TTTTTGCTTAGCATGGTGGAAA | ACG |
| 4309341 | CCGGGCCTTCCCTAGGTT | ACG |
| 4329858 | CCCGCCACCACGCCCGG | ACT |
| 10690268 | GACTCCGTTAAAAAAAAAAAAAAAA | ACT |
| 2401023 | TCTAAACTTGATAAGGCAAC | ACT |
| 1054745 | TTGGTCCATTAGGGAATTAGA | ACG |
| 8019453 | GGGAAATACTAGGAATGAATACT | ACT |
| 12431510 | GGCATGGTGGCGGGTGC | ACT |
| 7141067 | CATTGGCCGGGCATGGTG | ACT |
| 12431547 | TAGGGGAGAGCTAAAAACAAC | ACG |
| 7142316 | TGATGGCAGCATCTTATTACAT | ACG |
| 1959029 | CTTGAATTCTCATTGAGCATCT | ACG |
| 1959028 | TAATCCAACTCGCCTTCTCC | ACT |
| 11376292 | TAAAAAGTGGTAGACTTTTTTTTTT | ACG |
| 11376755 | AAGAATTTGCCCAGAAAAAAAAAA | ACT |
| 7145420 | ACCCGAAGCTAGGGTCGC | ACT |
| 8013347 | GAATATTCTATGATGTTAGGTGT | CGT |
| 2144070 | GAGGTGCTGGGGAGTGTGT | ACT |
| 11850135 | GCAAACATCCCCGGGAGC | ACG |
| 11850138 | CCCGGGGATGTTTGCTGAA | ACT |
| 1955617 | GATTTAAATGGGAGTATGATGTA | ACG |
| 11621899 | GGGGATGGTGAATGACCTCT | ACT |
| 4900508 | AGAGTGACCATTCCTATGTTC | CGT |
| 4906116 | TGCGTGGTTGGATGTTTTTCA | ACT |
| 4906117 | GAAGAAGGACGGTGACTCG | ACG |
| 2235972 | TCCCCCATCAGGACACC | ACG |
| 2235973 | GGTCAAGGTTTCAAAAATGG | ACT |
| 2235974 | GAGAAGAAAGAGAGAAAGCTTTTA | CGT |
| 2235975 | TGCGAACGATCCATCTA | ACG |
| 7149416 | TTTCCCCCACTGTCTCTGG | ACT |
| 11850809 | GGTCCCTGGTCTCTGGTTTT | ACT |
| 10140476 | ATGCGAGCTGGATTTTTAAACA | ACT |
| 12437309 | GAAGACTGTGGGAGTTTTGG | ACG |
| 6575852 | TGCGGCCAGGCAGAGCC | ACT |
| 4906118 | CAGTTAGGATGCGGGAGGC | ACT |
| 11428825 | CTTTCTCCACACCATTTTTTTTT | ACG |
| 11450744 | GATATTCCCTTGGTTAAAAAAAAAA | CGT |
| 1999612 | AGGTTTTGTGTCATCTAGC | ACT |
| 1889368 | CCTGCCACCCTGATATC | ACG |
| 1889367 | GCCACCCTGATATCTCTTA | ACT |
| 1889366 | CTTACTCCCTAAGACACTTG | ACG |
| 3077388 | CGGGCAATGGGCAGAGAG | CGT |
| 1889365 | AAACATTGACACCCCAAGTTC | ACG |
| 1955615 | CACTACCCTGATCTCACACA | ACT |
| 1569807 | AAGGCAGTTTGGAGAGC | ACT |
| 2064491 | AGCTAAGTGCCAAGATG | ACG |
| 2064492 | GGCACCAGGAGCAGGTCCTC | ACT |
| 761532 | TTCTGTCACTCACCCCC | ACG |
| 1955616 | GACAGGGTCTTTGCACAC | ACT |
| 2144065 | AGGCCGCCTCTTTATCA | ACG |
| 2144066 | AATTTCAGTGTCAACTTGG | ACG |
| 2895908 | TCTCTTCTTATAAGGGCACT | ACT |
| 2401043 | TAGCTTTATAGGACAGGTCTAG | CGT |
| 1007904 | CAGTTGTTTGTCTCTCTTGCCCA | ACT |
| 3891113 | AGAAAGGAGACTGGTTTGGG | ACT |
| 3891114 | CTGTCTAGTCTGTTCACTTTC | ACT |
| 730812 | AGCGATCCTCCCACCTC | ACT |
| 1884068 | CAGAATAAGGGCCAACT | ACT |
| 1884069 | ATAAGGGCCAACTGATGTAT | ACG |

### Genetic Analysis of Allelotyping Results

Allelotyping results are shown for cases and controls in Table 48. The allele frequency for the A2 allele is noted in the fifth and sixth columns for breast cancer pools and control pools, respectively, where “AF” is allele frequency. SNPs with blank allele frequencies were untyped.

TABLE 48

| | | | | | | | | Breast |
|---|---|---|---|---|---|---|---|---|
| | Position in | | | | | | | Cancer |
| | SEQ ID | Chromosome | A1/A2 | | | | | Associated |
| dbSNP rs# | NO: 7 | Position | Allele | Case AF | Control AF | p-Value | OR | Allele |
| 2895907 | 225 | 99854375 | A/C | A = 0.342 | A = 0.364 | 0.464 | 1.10 | C |
| | | | | C = 0.658 | C = 0.636 | | | |
| 2401019 | 505 | 99854655 | G/A | G = 0.282 | G = 0.303 | 0.477 | 1.10 | A |
| | | | | A = 0.718 | A = 0.697 | | | |
| 2401020 | 5170 | 99859320 | T/C | T= | T = 0.239 | | | |
| | | | | C= | C = 0.761 | | | |
| 2401021 | 5183 | 99859333 | T/C | T = 0.681 | T = 0.66 | 0.503 | 0.91 | T |
| | | | | C = 0.319 | C = 0.340 | | | |
| 1999611 | 6839 | 99860989 | T/C | T = 0.951 | T = 0.94 | 0.489 | 0.80 | T |
| | | | | C = 0.049 | C = 0.060 | | | |
| 1999610 | 7016 | 99861166 | C/T | C = 0.598 | C = 0.592 | 0.842 | 0.97 | C |
| | | | | T = 0.402 | T = 0.408 | | | |
| 1999609 | 7237 | 99861387 | C/T | C = 0.434 | C= | | | |
| | | | | T = 0.566 | T= | | | |
| 2275016 | 8190 | 99862340 | C/T | C = 0.609 | C = 0.642 | 0.316 | 1.15 | T |
| | | | | T = 0.391 | T = 0.358 | | | |
| 2275015 | 8357 | 99862507 | C/T | C = 0.291 | C = 0.31 | 0.524 | 1.09 | T |
| | | | | T = 0.709 | T = 0.690 | | | |
| 3818772 | 8577 | 99862727 | C/G | C = 0.705 | C = 0.665 | 0.177 | 0.83 | C |
| | | | | G = 0.295 | G = 0.335 | | | |
| 10149458 | 15999 | 99870149 | C/T | C = 1 | C = 1 | 0.913 | 0.00 | C |
| | | | | T = 0 | T = 0.000 | | | |
| 7142383 | 17722 | 99871872 | C/T | C = 0.991 | C = 0.972 | 0.0879 | 0.31 | C |
| | | | | T = 0.009 | T = 0.028 | | | |
| 11343023 | 18543 | 99872693 | T/— | T = 1 | T = 1 | 1 | | |
| | | | | — = 0 | — = 0 | | | |
| 11160641 | 21639 | 99875789 | C/A | C = 1 | C = 0.999 | 0.921 | 0.44 | C |
| | | | | A = 0.000 | A = 0.001 | | | |
| 12586334 | 24753 | 99878903 | C/A | C = 0.435 | C = 0.393 | 0.259 | 0.84 | C |
| | | | | A = 0.565 | A = 0.607 | | | |
| 10142208 | 26289 | 99880439 | G/A | G = 0.957 | G = 0.972 | 0.505 | 1.55 | A |
| | | | | A = 0.043 | A = 0.028 | | | |
| 7493230 | 27120 | 99881270 | C/A | C = 0.001 | C = 0.001 | 0.939 | 5.70 | A |
| | | | | A = 0.999 | A = 0.999 | | | |
| 5811036 | 28737 | 99882887 | A/— | A = 0.511 | A = 0.536 | 0.424 | 1.11 | — |
| | | | | — = 0.489 | — = 0.464 | | | |
| 10151926 | 29562 | 99883712 | T/A | T = 0.001 | T = 0.001 | 0.988 | 0.32 | T |
| | | | | A = 0.999 | A = 0.999 | | | |
| 12435354 | 30078 | 99884228 | T/C | T = 1 | T = 1 | 0.998 | 1.10 | C |
| | | | | C = 0.000 | C = 0.000 | | | |
| 7151007 | 30974 | 99885124 | C/T | C = 0.535 | C = 0.496 | 0.267 | 0.85 | C |
| | | | | T = 0.465 | T = 0.504 | | | |
| 11160642 | 31545 | 99885695 | T/C | T = 1 | T = 0.999 | 0.896 | 0.46 | T |
| | | | | C = 0.000 | C = 0.001 | | | |
| 11160643 | 31779 | 99885929 | T/A | T = 0.553 | T = 0.5 | 0.0966 | 0.81 | T |
| | | | | A = 0.447 | A = 0.500 | | | |
| 11626687 | 34062 | 99888212 | T/C | T= | T = 0.54 | | | |
| | | | | C= | C = 0.460 | | | |
| 4906112 | 34831 | 99888981 | T/G | T = 1 | T = 1 | 0.991 | 0.76 | T |
| | | | | G = 0.000 | G = 0.000 | | | |
| 8016071 | 34906 | 99889056 | G/C | G = 0.224 | G = 0.273 | 0.15 | 1.31 | C |
| | | | | C = 0.776 | C = 0.727 | | | |
| 11623698 | 34934 | 99889084 | T/G | T = 1 | T = 1 | 0.968 | 0.68 | T |
| | | | | G = 0.000 | G = 0.000 | | | |
| 7401628 | 34938 | 99889088 | T/G | T = 1 | T = 1 | 1 | | |
| | | | | G = 0 | G = 0 | | | |
| 8015995 | 35131 | 99889281 | G/A | G = 0.547 | G = 0.562 | 0.643 | 1.06 | A |
| | | | | A = 0.453 | A = 0.438 | | | |
| 10873526 | 35511 | 99889661 | T/C | T = 0.579 | T = 0.548 | 0.334 | 0.88 | T |
| | | | | C = 0.421 | C = 0.452 | | | |
| 11160644 | 35708 | 99889858 | A/G | A = 0.661 | A = 0.673 | 0.715 | 1.05 | G |
| | | | | G = 0.339 | G = 0.327 | | | |
| 10134252 | 36071 | 99890221 | G/C | G = 0.003 | G = 0.004 | 0.829 | 1.84 | C |
| | | | | C = 0.997 | C = 0.996 | | | |
| 1959030 | 36824 | 99890974 | A/T | A = 0.029 | A = 0.03 | 0.908 | 1.06 | T |
| | | | | T = 0.971 | T = 0.970 | | | |
| 9707396 | 36959 | 99891109 | T/G | T = 0 | T = 0 | 1 | | |
| | | | | G = 1 | G = 1 | | | |
| 2401022 | 37335 | 99891485 | A/T | A = 0.561 | A = 0.563 | 0.942 | 1.01 | T |
| | | | | T = 0.439 | T = 0.437 | | | |
| 4622448 | 37343 | 99891493 | A/G | A = 0.001 | A = 0.001 | 0.993 | 1.32 | G |
| | | | | G = 0.999 | G = 0.999 | | | |
| 10141019 | 38507 | 99892657 | C/T | C = 1 | C = 1 | 0.974 | 0.71 | C |
| | | | | T = 0.000 | T = 0.000 | | | |
| 10141096 | 38554 | 99892704 | A/C | A = 0.002 | A = 0.003 | 0.908 | 1.74 | C |
| | | | | C = 0.998 | C = 0.997 | | | |
| 11621063 | 38624 | 99892774 | C/T | C = 0.5 | C = 0.445 | 0.11 | 0.80 | C |
| | | | | T = 0.500 | T = 0.555 | | | |
| 12434632 | 38690 | 99892840 | T/C | T = 0.371 | T = 0.382 | 0.726 | 1.05 | C |
| | | | | C = 0.629 | C = 0.618 | | | |
| 12100901 | 38744 | 99892894 | T/C | T = 0.166 | T = 0.211 | 0.136 | 1.34 | C |
| | | | | C = 0.834 | C = 0.789 | | | |
| 12434655 | 38756 | 99892906 | T/C | T = 0.408 | T = 0.428 | 0.532 | 1.09 | C |
| | | | | C = 0.592 | C = 0.572 | | | |
| 9944103 | 39525 | 99893675 | G/A | G = 0.816 | G = 0.819 | 0.924 | 1.02 | A |
| | | | | A = 0.184 | A = 0.181 | | | |
| 7153189 | 40682 | 99894832 | G/A | G = 0.001 | G = 0.001 | 0.986 | 1.43 | A |
| | | | | A = 0.999 | A = 0.999 | | | |
| 7158860 | 41181 | 99895331 | A/G | A = 0.001 | A = 0.001 | 0.943 | 0.22 | A |
| | | | | G = 0.999 | G = 0.999 | | | |
| 12437080 | 41414 | 99895564 | G/C | G = 0.883 | G = 0.817 | 0.0081 | 0.59 | G |
| | | | | C = 0.117 | C = 0.183 | | | |
| 11851768 | 41657 | 99895807 | G/A | G = 0.211 | G = 0.23 | 0.471 | 1.12 | A |
| | | | | A = 0.789 | A = 0.770 | | | |
| 12431943 | 42821 | 99896971 | G/A | G = 0.239 | G = 0.314 | 0.0245 | 1.46 | A |
| | | | | A = 0.761 | A = 0.686 | | | |
| 4309341 | 45475 | 99899625 | G/A | G = 0.999 | G = 0.999 | 0.924 | 0.59 | G |
| | | | | A = 0.001 | A = 0.001 | | | |
| 4329858 | 45611 | 99899761 | G/C | G = 0.999 | G = 0.999 | 1 | 1.00 | |
| | | | | C = 0.001 | C = 0.001 | | | |
| 10690268 | 45763 | 99899913 | AAA/— | AAA = 0.454 | AAA= | | | |
| | | | | — = 0.546 | —= | | | |
| 2401023 | 46038 | 99900188 | T/C | T = 0.731 | T = 0.773 | 0.178 | 1.25 | C |
| | | | | C = 0.269 | C = 0.227 | | | |
| 1054745 | 46344 | 99900494 | G/A | G = 0.188 | G = 0.27 | 0.00228 | 1.60 | A |
| | | | | A = 0.812 | A = 0.730 | | | |
| 8019453 | 46669 | 99900819 | T/C | T = 0.116 | T = 0.249 | 0.000607 | 2.52 | C |
| | | | | C = 0.884 | C = 0.751 | | | |
| 12431510 | 46926 | 99901076 | G/C | G = 0.999 | G = 0.999 | 0.98 | 1.17 | C |
| | | | | C = 0.001 | C = 0.001 | | | |
| 7141067 | 47069 | 99901219 | T/C | T = 0.001 | T = 0.002 | 0.97 | 1.45 | C |
| | | | | C = 0.999 | C = 0.998 | | | |
| 12431547 | 47293 | 99901443 | G/A | G = 0.192 | G = 0.294 | 0.000196 | 1.76 | A |
| | | | | A = 0.808 | A = 0.706 | | | |
| 7142316 | 47602 | 99901752 | G/A | G = 0.96 | G = 0.993 | 0.0544 | 5.62 | A |
| | | | | A = 0.040 | A = 0.007 | | | |
| 1959029 | 49821 | 99903971 | G/A | G = 0.898 | G = 0.842 | 0.01 | 0.60 | G |
| | | | | A = 0.102 | A = 0.158 | | | |
| 1959028 | 49875 | 99904025 | T/C | T = 0.941 | T = 0.873 | 0.000419 | 0.43 | T |
| | | | | C = 0.059 | C = 0.127 | | | |
| 11376292 | 51178 | 99905328 | —/A | — = 0.322 | — = 0.31 | 0.68 | 0.94 | — |
| | | | | A = 0.678 | A = 0.690 | | | |
| 11376755 | 51180 | 99905330 | A/— | A = 0.794 | A = 0.785 | 0.742 | 0.95 | A |
| | | | | — = 0.206 | — = 0.215 | | | |
| 7145420 | 51772 | 99905922 | A/G | A = 0.301 | A = 0.291 | 0.73 | 0.95 | A |
| | | | | G = 0.699 | G = 0.709 | | | |
| 8013347 | 51912 | 99906062 | G/T | G = 0.16 | G = 0.249 | 0.000671 | 1.75 | T |
| | | | | T = 0.840 | T = 0.751 | | | |
| 2144070 | 52252 | 99906402 | T/C | T = 0.774 | T = 0.735 | 0.224 | 0.81 | T |
| | | | | C = 0.226 | C = 0.265 | | | |
| 11850135 | 52294 | 99906444 | G/A | G = 1 | G = 1 | 0.965 | 1.64 | A |
| | | | | A = 0.000 | A = 0.000 | | | |
| 11850138 | 52317 | 99906467 | A/G | A = 0.002 | A = 0.002 | 0.985 | 0.90 | A |
| | | | | G = 0.998 | G = 0.998 | | | |
| 1955617 | 54548 | 99908698 | G/A | G = 0.534 | G = 0.521 | 0.736 | 0.95 | G |
| | | | | A = 0.466 | A = 0.479 | | | |
| 11621899 | 54889 | 99909039 | T/C | T = 0.004 | T = 0.009 | 0.774 | 2.36 | C |
| | | | | C = 0.996 | C = 0.991 | | | |
| 4900508 | 55377 | 99909527 | C/A | C = 0 | C = 0 | 1 | | |
| | | | | A = 1 | A = 1 | | | |
| 4906116 | 55498 | 99909648 | T/C | T = 0.038 | T = 0.027 | 0.619 | 0.70 | T |
| | | | | C = 0.962 | C = 0.973 | | | |
| 4906117 | 55622 | 99909772 | C/T | C = 0.327 | C = 0.276 | 0.0901 | 0.79 | C |
| | | | | T = 0.673 | T = 0.724 | | | |
| 2235972 | 55799 | 99909949 | C/T | C = 0.726 | C = 0.658 | 0.0193 | 0.73 | C |
| | | | | T = 0.274 | T = 0.342 | | | |
| 2235973 | 55995 | 99910145 | T/C | T = 0.163 | T = 0.235 | 0.0297 | 1.58 | C |
| | | | | C = 0.837 | C = 0.765 | | | |
| 2235974 | 56042 | 99910192 | C/A | C = 0.163 | C = 0.245 | 0.00633 | 1.66 | A |
| | | | | A = 0.837 | A = 0.755 | | | |
| 2235975 | 56342 | 99910492 | G/A | G = 0.948 | G = 0.967 | 0.184 | 1.61 | A |
| | | | | A = 0.052 | A = 0.033 | | | |
| 7149416 | 57781 | 99911931 | T/C | T = 0.662 | T = 0.717 | 0.178 | 1.29 | C |
| | | | | C = 0.338 | C = 0.283 | | | |
| 11850809 | 57983 | 99912133 | A/C | A = 0.002 | A = 0.002 | 0.956 | 0.74 | A |
| | | | | C = 0.998 | C = 0.998 | | | |
| 10140476 | 58906 | 99913056 | T/C | T = 0.93 | T = 0.908 | 0.298 | 0.75 | T |
| | | | | C = 0.070 | C = 0.092 | | | |
| 12437309 | 59257 | 99913407 | C/T | C = 1 | C = 1 | 0.954 | 3.60 | T |
| | | | | T = 0.000 | T = 0.000 | | | |
| 6575852 | 60926 | 99915076 | T/G | T = 0.998 | T = 0.999 | 0.875 | 2.34 | G |
| | | | | G = 0.002 | G = 0.001 | | | |
| 4906118 | 61462 | 99915612 | A/G | A = 0.892 | A= | | | |
| | | | | G = 0.108 | G= | | | |
| 11428825 | 62241 | 99916391 | —/T | — = 0.6 | — = 0.57 | 0.396 | 0.88 | — |
| | | | | T = 0.400 | T = 0.430 | | | |
| 11450744 | 62541 | 99916691 | —/T | — = 0.574 | — = 0.545 | 0.387 | 0.89 | — |
| | | | | T = 0.426 | T = 0.455 | | | |
| 1999612 | 63600 | 99917750 | T/C | T = 0.006 | T = 0.005 | 0.916 | 0.82 | T |
| | | | | C = 0.994 | C = 0.995 | | | |
| 1889368 | 63978 | 99918128 | G/A | G = 0.429 | G = 0.471 | 0.186 | 1.18 | A |
| | | | | A = 0.571 | A = 0.529 | | | |
| 1889367 | 63983 | 99918133 | G/C | G = 0.836 | G = 0.82 | 0.562 | 0.90 | G |
| | | | | C = 0.164 | C = 0.180 | | | |
| 1889366 | 63999 | 99918149 | G/A | G = 0.325 | G = 0.331 | 0.822 | 1.03 | A |
| | | | | A = 0.675 | A = 0.669 | | | |
| 3077388 | 64042 | 99918192 | —/CT | — = 0.607 | — = 0.563 | 0.183 | 0.84 | — |
| | | | | CT = 0.393 | CT = 0.437 | | | |
| 1889365 | 64276 | 99918426 | G/A | G = 0.721 | G = 0.742 | 0.461 | 1.12 | A |
| | | | | A = 0.279 | A = 0.258 | | | |
| 1955615 | 66308 | 99920458 | A/G | A = 0.636 | A = 0.615 | 0.5 | 0.91 | A |
| | | | | G = 0.364 | G = 0.385 | | | |
| 1569807 | 67400 | 99921550 | T/C | T = 0.694 | T = 0.687 | 0.812 | 0.97 | T |
| | | | | C = 0.306 | C = 0.313 | | | |
| 2064491 | 69255 | 99923405 | C/T | C = 0.406 | C = 0.373 | 0.298 | 0.87 | C |
| | | | | T = 0.594 | T = 0.627 | | | |
| 2064492 | 69263 | 99923413 | T/C | T = 0.343 | T= | | | |
| | | | | C = 0.657 | C= | | | |
| 761532 | 70965 | 99925115 | C/T | C = 0.73 | C = 0.792 | 0.0607 | 1.41 | T |
| | | | | T = 0.270 | T = 0.208 | | | |
| 1955616 | 71540 | 99925690 | A/G | A = 0.546 | A = 0.602 | 0.097 | 1.26 | G |
| | | | | G = 0.454 | G = 0.398 | | | |
| 2144065 | 74698 | 99928848 | G/A | G = 0.38 | G = 0.35 | 0.33 | 0.88 | G |
| | | | | A = 0.620 | A = 0.650 | | | |
| 2144066 | 74746 | 99928896 | C/T | C = 0.567 | C = 0.543 | 0.458 | 0.91 | C |
| | | | | T = 0.433 | T = 0.457 | | | |
| 2895908 | 75221 | 99929371 | A/G | A = 0.426 | A = 0.358 | 0.0304 | 0.75 | A |
| | | | | G = 0.574 | G = 0.642 | | | |
| 2401043 | 91289 | 99945439 | T/A | T = 0.001 | T = 0 | 0.948 | 0.00 | T |
| | | | | A = 0.999 | A = 1 | | | |
| 1007904 | 91796 | 99945946 | A/G | A = 0.514 | A = 0.526 | 0.732 | 1.05 | G |
| | | | | G = 0.486 | G = 0.474 | | | |
| 3891113 | 93762 | 99947912 | C/G | C = 0 | C = 0 | 1 | | |
| | | | | G = 1 | G = 1 | | | |
| 3891114 | 93780 | 99947930 | G/C | G = 1 | G = 1 | 0.976 | | C |
| | | | | C = 0.000 | C = 0 | | | |
| 730812 | 94242 | 99948392 | T/G | T = 0.903 | T = 0.888 | 0.57 | 0.86 | T |
| | | | | G = 0.097 | G = 0.112 | | | |
| 1884068 | 94809 | 99948959 | A/G | A = 0.558 | A = 0.548 | 0.748 | 0.96 | A |
| | | | | G = 0.442 | G = 0.452 | | | |
| 1884069 | 94816 | 99948966 | C/T | C = 0.288 | C = 0.256 | 0.274 | 0.85 | C |
| | | | | T = 0.712 | T = 0.744 | | | |

FIG. 1G shows proximal SNPs in and around the LOC145197 region for females. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1G can be determined by consulting Table 48. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the gene or genes present in the loci region of the proximal SNPs as annotated by Locus Link (accessible on the World Wide Wed at the URL “ncbi.nlm.nih.gov/LocusLink/”) are provided on the graph. The exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

Provided hereafter are ICAM, MAPK10, KIAA0861, NUMA1, GALE, DPF3 and LOC145197 genomic sequences (SEQ ID NO: 1, 2, 3, 4, 5, 6 and 7, respectively). Polymorphic variants are designated in IUPAC format. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

Following is an ICAM genomic nucleotide sequence that encodes ICAM1, ICAM4 and ICAM5 (SEQ ID NO: 1).

| >19:10219601-10312200 | |
|---|---|
| 1 | aggaggctga ggtgggagaa tggcgtgaac ctgggaggcg gagcttgcag tgagccaaga |
| 61 | tcacgccact gcactccagc ctgggcgaca gagcgagact ccgtctcaaa aaaaaaaaaa |
| 121 | aaaaaaaaaa agccaggcga ggtgactcac acccataatc gcagcacttt gggaggccaa |
| 181 | ggcgggcaga tcgcttgagc ccagaagttt gagatctgcc tgggcaacaY ggcgagaccc |
| 241 | tgtctctatg aaacaagaaa aaagaaagaa acgaaatact gttccatcat ttgccacata |
| 301 | aaatctaaac tctctggtgc agaacagact tgcaaatctg cctgtaaaat catacaccgg |
| 361 | attttggagt agacacacag gaggctgatt gtagtggtgg cttctgggag aagaactggg |
| 421 | gggtaggggg cacagataag agtagacaga attttcactc tagtaatctt tgtaattttt |
| 481 | gaaattctct ctaccaggtg tatgaatgac caatgaaaat atacaatagg ccaggcgcag |
| 541 | tggctcacgc ctataatccc agcactttgg gaggctgagg cgggtggatc acatgaggtc |
| 601 | aggagtccga ggccagcctg accaacatgg tgaaaccttg tctctacgaa aaatacaaac |
| 661 | attagtcagg tagggagcat cacttgagaa caggagttca agaccagcct ggacaacaaa |
| 721 | gcaagacctc tgcctctaca aaaaaaaaaa aaaaaaaaaa aaattgctgg gcatggtgga |
| 781 | acatgcctgt ggcctcagct acttaggtgg ctgcagcaga ggatcatctg aggccaggaa |
| 841 | ttcaagacta cagtgaacta taatcgttac actgcactac agactggtaa cagggtgaga |
| 901 | tcctgtctca aaaacaacag caacaaaata aacgattaaa agacctttta aagttgcaaa |
| 961 | atacatagag tcttaaacat gcatccacag ttatttattt atttatttag aaacggagtt |
| 1021 | tcactcttgt tgcccaggct ggagtgcaat ggtgcggtat cagctcactg caacctccgc |
| 1081 | ctcccaagtt aaaggaattc tgcctcagcc ttccaagtag ctggaattac aggcatgcgc |
| 1141 | caccatgctc ggctaatttt gtatttttag taaagatggg gttttggcat attggccagg |
| 1201 | atggtttcga acccctgacc tcaggtgatc cacccgcctc ggactcccaa agtgctagga |
| 1261 | ttataggcat gagccaccac gcccggccgc attcagttat tcttgcaggg ataaaataca |
| 1321 | caatgaaata cactatactt tttgttttct ttctttcttt tttttttttt tttttgagac |
| 1381 | agagtctcac tcgtcgcatg ggctggagtg cggtgcggga tctcggctca ctgcaacctc |
| 1441 | tgcctcctgg gttgaagcga ttctcctgcg tcagcctccc gagtagctgg gattacaggt |
| 1501 | gcccgccact acgcccagct aatttttttg tatttttagt agagataagg tttcaccatg |
| 1561 | ttggccaggc tggtctcgaa ctcctgactc gtgattcgcc caccttggcc tcccaaagtg |
| 1621 | ctgggattac aggcgtgagc caccgcaccc ggccacattt tttttttttt ttttttgaga |
| 1681 | caggatctta gtctgtcacc caggctggag tacagtggca tgaacatggc tcactgcagc |
| 1741 | ctcaatctct tgggttcagg tgatccttct gccttagcct ccccattagc tgggaccaca |
| 1801 | ggcatatacc atcacaccta gctaattttt aaatttttgg tagaggccag gcgcggtggc |
| 1861 | tcacgcctgt aatcccagca ctttgggagg ccgagtcggg tggatcacct gaggtcggga |
| 1921 | gttcgagacc agcctggcca aaatggtgaa accctgtctc tactaaaaat aaaaaataaa |
| 1981 | aataaaaatt agctgggcgt agtggcgggc gcctgtaatc ccagctactc gggaggctga |
| 2041 | ggcaggagaa tcgcttgaac ccaggaggca gaggttgcag tgagccaaga tcgcgctact |
| 2101 | gcactccagc ctgggcaaca gagtgagatt ctgtctcaaa aaaaaaataa taataatgct |
| 2161 | atttattgac tttacacctt gtaccaggca tgggaagctt tgcctccatt acgtcactga |
| 2221 | atctcataac ctccttttcc agcagaggaa aatgaggttg gttcacagac cacgttgtca |
| 2281 | gctgtgctct gtccagaacg cactggcctc caagtagaca gccctggact ggtagggaag |
| 2341 | ccggctatga tccggtggcg ccccctggag gtctatcggg aacatggtaa agaacctaaa |
| 2401 | aatgggtggg ccacagtagc tcatgcctgt aatcccagca ctttggggga ccaatgcggg |
| 2461 | agaattgctt gagcccagga gttcaagacc agcctgggca acattgggag acccaccccc |
| 2521 | cgccatctct acaaaaaaaa aattaggccg ggcgcggtgg ctcaggcctg taatcccagc |
| 2581 | actttgggag gccgaggcgg gtggatcacc tgaggtcagg agttcaagac cagcctggcc |
| 2641 | aacgtggtgc cacactgtct ctaataaaaa cacgaaaatt agggccgggc tcggtggctc |
| 2701 | acgcctgtaa tcccagcact ttgggaagcc gaggcgggcg gatcacgagg tcaggagatt |
| 2761 | gagaccatcc tggctaacac ggtgaaaccc cctctctact aaaaatacaa aaagttagcc |
| 2821 | aggtgtggtg gcgggcgcct gtagtcccag ctactcggta agctgaggca gggaatcgct |
| 2881 | tgaacccggg aggccgagat gtgcgatctc acaccactgc actccggcct gggcgacaga |
| 2941 | gcgatactcc atctcaaaat acaaatacaa aataaaaaaa tacaatacaa aaatgagccg |
| 3001 | ggcttgcgta cctgtagacc agctactcag gagacagagg caggagaatc ccttgagctc |
| 3061 | tggaggtcga ggctgcagta agccatgatc ttgcccattg cactccagcc tgggcgacag |
| 3121 | aggaagacct tgtctctaaa aaccaaaaca aagaaactaa aaataagcat tcggatttgt |
| 3181 | tagggggcga caagggaggc actccaggat ctgtggactc cccactttgt tctgtccttg |
| 3241 | gagagccctg gaaggtctga gaggggacgg gacctggttt aagggggtag gggagaggac |
| 3301 | cctggtctag ggggtaaggg acacaaatac ctaatctgag gggtttcggg gggacatggc |
| 3361 | cctgccctgg ggaatctaaa ctgggaggca tggtctggca ggcccaatcc tgaaggcttt |
| 3421 | ctgagggcaa taaggccctg cctttaaaca aatgaaaaaa cgccaggcac ggtggctcac |
| 3481 | gcctgtaatc ccagcacttt ggaaggctga ggcgggagga tcacgaggtc aggagatcga |
| 3541 | gaccatcctg gctaacgcgg tgatacccgt ctctactaaa aatacaaaaa attagccagg |
| 3601 | cgtggtggcg ggcgcctgca gtcccagcta ctcgggaggc tgaggcagga gaatggcgtg |
| 3661 | aacccaggag gcggagcttg cagtgagccg agatcgcgcc actgcactcc agcctgggca |
| 3721 | acagagcaag actccgtctc aaaaaaaaca aaaacaaaaa caaaaacaaa aacaaaaaaa |
| 3781 | caaataaaaa aacaaaacca cataactgga taaagaaaaa accatctccg acagccttgg |
| 3841 | aggcgggatg aaggcgtggc ccggtgggcg tgaccaacag caaaagttta agcgttattg |
| 3901 | gctgtattcc ttagttgctc actccagaac tgcccactta tgggcggggg tcactccttc |
| 3961 | aggcttaaca gtcattggct gaattgggcc agagaggtct cattggctga attcctgcac |
| 4021 | cggctcgtcg gaggcgggac ccaaagtagg ctaggcctac ggaagctggg tcttcttgct |
| 4081 | gtgaggtcgc gttccccagt gttacggagg gtccttgagg caggagtgaa aattgggtct |
| 4141 | gggggttagt cctggggtgg aggtctgggc acgccgggtc ggaccccctc catcttcggt |
| 4201 | tttgcacacc ccgctttcca gcgcggagtc gcggcgggta gggcggcgtc gcgtgcgtga |
| 4261 | cgtcatccag cggcgcctcg cRaggctcca gtggccttga cctcccgcgg cgtgggaggc |
| 4321 | tgcgcggcga tgctgcagtt cgtccgggcc ggggcgcggg cctggcttcg gcctaccggc |
| 4381 | agccaggtga ggccaggggc tggaggcgtg gtcgaaggat gaaatttggg ggtgtccagg |
| 4441 | ggtcgtctct cactttcgcc caacccttgc agggcctgag ttccctggcg gaagaggcag |
| 4501 | cgcgtgcgac cgagaacccg gagcaggtgg cgagcgaggg taaggcaacc ggggtggctc |
| 4561 | caggaggggc ggcgacagag aggtctgacc cttgacccta acctctgacc cccgcaatcg |
| 4621 | ctccaggtct cccggagccc gtgctgcgca aagtcgagct cccggtaccc actcatcgac |
| 4681 | gcccagtgca ggcctgggtc gagtccttgc ggggcttcga gcaggagcgc gtgggcctgg |
| 4741 | ccgacctgca ccccgatgtt ttcgccaccg cgcccaggtg agcgagggct gtaatggtga |
| 4801 | actgagtggc agagggatga agagcgggat ttcaggagtc acgatgactt tgggcttgta |
| 4861 | cccttgggaa agtgctgtat ttctacagcc tccgtttctc cacctgtcaa aggggaatga |
| 4921 | tgacagtttc ccctgctgta gcgctgtgtg agattgaagc ctgagaggtg acatcatcta |
| 4981 | agggttaggg agacagaatt ctggagcccg actgattagg ttcaaatcct gccttcccct |
| 5041 | cttgtccctc agtgtcccta ttttgtcagc ggtcgggagg ttgctgtgat gaataaatga |
| 5101 | cttaattctg gcacataata agttctatag aaatgttgat aatctttgtt aactggtttt |
| 5161 | tgcaaataag agcactaaaa agactaaacc attcctcggt gcctggaaga ggctgtttgc |
| 5221 | attttagtta ccctgctgtt cataacatct ctaagaaaat gtaggggcca ccctgggcgc |
| 5281 | agtggctcac gcctgtaatc ccagcacttt gggaggccaa ggcgggcgga tcacgaggtc |
| 5341 | aggagatcga gaccatcctg gctaacatgg tgaaaccccg tctctactaa aaatacaaaa |
| 5401 | aaaaattagc cgggcgtggt ggcgggcgtc tgtagtccca ggtactctgg aggctgaggc |
| 5461 | aagagaatgg cgtgaacccg ggaggcggag cttgcagtga gccgagatcg cgccactgca |
| 5521 | ctccatcctg ggcaacactc tgtctcaaaa aaaaaaaaaa aaaagaaaga aaagaaaatg |
| 5581 | taggggccag ttactgtggc tcacatctgt aatcccagca cattgggagg ccgaggtggg |
| 5641 | cggatcactt gaaaccagga gttgcagacc agcctggcca acatgatgaa accccgtctc |
| 5701 | taccaaaaat acaaaaatta gccggacgtc ttagtgcaag cctgtagtcc cagctactca |
| 5761 | ggaggctgag gcatgagaat cgcttgcacc tggggagatg gaggttgcag tgagccgtga |
| 5821 | ttatactact gcactccagc ctgggcgaca gagacagact ccatctcaaa aaaaaaaaca |
| 5881 | ggtgaaattg atttcaataa tgtatttaac ctgtatttaa aactatgtcg aaatcacatt |
| 5941 | gtagcatggg gcactggcca tgtttcagat gttgagtatg tgactgtatg gaatggtata |
| 6001 | gaactagaga ggaaaaccag tccctgaaga aggtggcaat aagtgaagtg taatagcagg |
| 6061 | aaaaaagtaa tggtaggaaa aacaaggaag aaggggtggc tttttttttc tgagatgaag |
| 6121 | tttcgctctt gtcgcccagg ctggagtgca atggcatgct ctcagctcac ttcaacctcc |
| 6181 | gcctcctggg ttctactaat tctcatgcct cagcctcccg agtagccaga atgacagaca |
| 6241 | tgtaccaccg tgcccagcta atctttgtat attttggaga gacatcactt tgccatgttg |
| 6301 | cccaggctgg tcttactcct tgcctcaagt gatccacctg ccttggcctc ccaaagtgcc |
| 6361 | aggattccag gcatgagcca ctgcacctgg tcagggtggc tctttcttta gaaggtacct |
| 6421 | ttcagcagtt attggagctg ctacctgtag gctgagaaag aaccatccag gagaagagtg |
| 6481 | tttcaggcag agggaacagc aagtgccaag gccctgaggc agaatttcaa gatggggtca |
| 6541 | gtgaggggca gaggcaaatc gcccagggcc ctggaggcag aagggagaat cctgggtttt |
| 6601 | cctatagtgg ggtgggagcg tttgaagcag agttgggctt ctcatgtgtc cttcctcccc |
| 6661 | gcaggctgga catactgcac caggttgcta tgtggcagaa gaacttcaag agaattgtga |
| 6721 | gtgcctaaat ggagcaaggt ggtgggaagg agcttcctgg ggaggttggg gataggaccc |
| 6781 | agaggaagcc catcgctggg ttttctctgg actgctcggc tggggcctca tctgtctcct |
| 6841 | gaactattca ccgatgggtc catttttggt tctctttttt ttgtttgttt ttgagatgga |
| 6901 | gatggagtct cactctgtca cccaggctgg agtgcagtgg cgcaatcttg gttcactgca |
| 6961 | acctccgctt cctgggttca agcgattctc ctgcctctgc ctcctgagta gctgggatta |
| 7021 | caggtgtgca ccaccatgca ggctaatttt tgtattttta gtagagatgg agtttcacca |
| 7081 | tgttggtcag gctggtctca aactcctgac ctcaaggaac ctacctgcct tggcctccca |
| 7141 | aagtgctggg attacaggcg tgagccaccg tgcccagctc attttcagtt gttttctgat |
| 7201 | cactcactga tgtgggcatt gtggtgggtg agaggatata gcagggacca cgaggacaaa |
| 7261 | acaggcaagg tccggctgtg tgcaagtggc tcaggcctgt aatcccagca ctggaagctg |
| 7321 | aggtgggtgg gttgcttggg gccaggaaag accagcctgg gaaacagcaa gacccagtct |
| 7381 | ctaccccttc ccccacaacc ccaagaaaaa gatgggcaaa gtccctattc taatgaaggt |
| 7441 | cacagtgtca tgggaggaga agtgaaggtg agtcagatgg tcatacgtaa tgtgtaatta |
| 7501 | tgagcatgtc ccagagaatg ggacgttcaa ccttgggtct actgtagaag ctaaccagat |
| 7561 | caaggaggca gtggggctag tgtggagata acaggaacag ggtgtgcaaa ggccctgtgg |
| 7621 | cagggaccac aaggaagcca gtgttgccac agaggccagt gggagcagtg gggaggagtc |
| 7681 | tgggttttgt gccaagaaca ttgaagaatc cattaccagt cagggtctta tccatccacc |
| 7741 | ccagaggtct ggccagtcct ccccctgctc acaccttccc tggctcccca tcaccctagg |
| 7801 | aataaagtca tcagtctgct attctgagtt cttcctgatt tccttgccct gctcctcttt |
| 7861 | cctgattctc cctgtctgtg tcatttccca tggccctccc tccttgtggc tacaaccaca |
| 7921 | ttaaaattgt tcaaaaaata ggccggcacg gtggctcaca cctgtaatct cagcactttg |
| 7981 | ggaggccaag gcaggtggat cacttgaggc caggagttcc aaaccagcct agtcaacgtg |
| 8041 | gcaaaacccc atctctactg aaaatacaaa aaattagctg gggccaggtg cggtggctca |
| 8101 | cacctgtaat cccagtactt tgggaggccg aggcggatgg atcacgaggt cgggagatcg |
| 8161 | agaccagcct gaccaatatg gtgaaacccc atctctacta aaaatacaaa tattagttgg |
| 8221 | gtgtggcagc gggcgcctgt agtcccagct actctggagg ctgagacagg agaattgctt |
| 8281 | gaacctggga ggcggaggtt gcagtgagcc gagatcgcgc cactgcactc cagcgtgggc |
| 8341 | gacagagcga gactccatct caaaaaaaaa aattagccgg gcatggtgac aggcacccgt |
| 8401 | aatctcagct actagggagg ctgaggcagg agaatcgctt gaatctagga atcagaggtt |
| 8461 | gcagtgagcc aaggtcacac cacttgcact ccacagcctg ggtgacagag tgagactgcc |
| 8521 | tcaaaaaaaa aaatcaatag ttcagaaaat accgagcacc ccctgcgtgc ccggtggcag |
| 8581 | ggcccctgcc tttgtgaggc tggcatgggg caggagctgt gacttcttta ccctccccct |
| 8641 | cgctccccaa gtactctgtg cttggccaga gagagcccgt catcatggtg cctcctgtct |
| 8701 | gacttccccg tggcaggact gatctgcccg gctccctgac acctgcctcg tggacctgac |
| 8761 | ccccctcctc tttgtgcctc cagagctatg ccaagaccaa gacgagagcc gaggtgcggg |
| 8821 | gcggtggccg gaagccttgg ccgcagaaag gcactgggcg ggcccggcat ggcagcatcc |
| 8881 | gctctccgct ctggcgagga ggtaacagga cagggtggag ggggcgggga ggggtggggg |
| 8941 | ggccagggaa gggcctgggt gtttactcac acacagctgc gcacatctgg catggtatta |
| 9001 | tgtcagccct gttccctcca cctcatggaa ccacctgggc tggtgacatc ggaactgagg |
| 9061 | cccttggacc tcactaccca tataagggga cagagatctg ggagccatcc actcctccct |
| 9121 | ctcgcatgcc ctgtctcttc agcctgtggc cacagcccct cttgacccca cctcctgtca |
| 9181 | ccctctcctg acccacactt cccacagcac ccagagagct cttctaaatc gtggaacctg |
| 9241 | aactccggac ctcggccttt gtgtggaacc tgaactcctg accttgtcat tgtgggccct |
| 9301 | ggtggctgca cacctttccc cctcatcccc tcctttccct ctctgaccag gcagagatga |
| 9361 | ctctctcttg ttttttcggt tgtgtttgtt tttaactttt tattttcttg aatgctaaca |
| 9421 | agatgactca gggtggtgcc agccacccca tcacctgttt acctggccag ctctcctcac |
| 9481 | ctttcagggt tctgggccac acctcctcca ggaagccccc cttgatctcc tttccttcca |
| 9541 | catcccccgg agctaccctg atttcttcta cagctgagcc tcttttctgc cctgccggaa |
| 9601 | tgtgaatggc atgagggcag ggaccatgtc tgttgtcttc tctgctgcat ttccaaggcc |
| 9661 | caggcgaggg cagacaccaa cacatggtgc ttgcaggggt ctccctgact gttgttgctc |
| 9721 | ccagatcata ctgcttgctc caccggagca tgtgcctgat gccttcctct cccgcttgac |
| 9781 | ctgaaagttc gaacctcctg ataacttcag cattaacagc gtgcttgagt taagttcaca |
| 9841 | ctctagccac tctatggaat ccacaccata actcatggtg tcctatgggg caggaactgt |
| 9901 | ccgatctcaa ggtggtttgt tttttttatt gtttgctttt gagacaagat ctcgctctgt |
| 9961 | tgcccaggct gtagtgcagt ggtgcaatca tagctcactg cagccttgag ctcctgggct |
| 10021 | caagtgatcc tccatcctca gcctcccaaa atgctggtgt gagccacctt cgccagccct |
| 10081 | gtctcagagt gtgacagctg ggaaaactga agcccagtga agcaaagtca ctggtcccat |
| 10141 | gttgtccctg atccagcctc ccctgaggcc ccaccctctc tgccttgttc ctggcagcgc |
| 10201 | cccctctctc gtcaccccat gccagccact gcagcagatt gctgacctcc aggctctgca |
| 10261 | gtggccccta cctgctcaca tgcctctgtc cccgcaggag gtgttgccca tggcccccgg |
| 10321 | ggccccacaa gttactacta catgctgccc atgaaggtgc gggcgctggg tctcaaagtg |
| 10381 | gcactgaccg tcaagctggc ccaggtacag ccatgggggg gcccagacag ctgctagagg |
| 10441 | tggggctgct ctggacccag ggttcaaacc atcctttcct tccaccagga cgacctgcac |
| 10501 | atcatggact ccctagagct gcccaccgga gacccacagt acctgacaga gctggcgcac |
| 10561 | taccgccgct ggggggactc cgtactcctc gtggacttgt gagggcacag ggcagagcag |
| 10621 | gggcaggggg ccctgagctc cgtactctga gggttcaacc cccactccct ggcctctctt |
| 10681 | acagaacaca cgaggagatg ccacagagca tcgtggaggc cacctctagg cttaagacct |
| 10741 | tcaacttgat cccggctgtt ggtgagcaaa gagcccaggc ccctagagtg cgcatgtgca |
| 10801 | ggctccgctg ttagaatcac agcggttcaa atccggcatc tggtcgctga gtggcctcag |
| 10861 | gcagtgacca cgctcccgga cccaaccttc agcttgccca aagcaataat ctttcctaaa |
| 10921 | gaagtgcttg gctggggatg gtggctcacg cttgtaatcc cagtactttg ggaggccaag |
| 10981 | gcagtctggg caatatagtg aggctcccat ctgtactaaa aataaaaaag ttaggcgtgg |
| 11041 | cgatgtgcac ctgtagtccc agctactcgg gggctgagcc aggcggatag cttgagct |
| 11101 | gggggccaag gctgcagtga gtcatgatcg caccactgca cccaaacctg gggagagagc |
| 11161 | tagactcttg tctcaaaaaa aaaaaaaaaa aaaaaaaaaa agctccaaag tcacctctgt |
| 11221 | caaagccaca gtctgttcct tccacagcca caagatggcg acatgagcct aagtcaagtc |
| 11281 | ctgtccctca acccacgccc ctcgctggct tccccacctc tctcaggatg aaagcccaag |
| 11341 | tcatcagggt ggcacgtcag gccctgcacg atgtgccccg tcacctccct gccctcccgt |
| 11401 | catttgccct gggtctcccc caccagaatg ggagccagga gtcccagcca ggcacaggac |
| 11461 | cgagcctggc tctcggtcag caggtgatga gctgggagca gctccttggc cagaggctta |
| 11521 | caggcaacaa gcggccaggc agggtctggc cccgggctgc tgggctcaca aagtcacact |
| 11581 | agaccacagt gacgatctct gtaagcacaa agggactccg atgtgggtgg ggtgaggaga |
| 11641 | gaggcagccc cggcctgacc ggccccccgc cccgccccca ccccgccccc aggcctaaat |
| 11701 | gtgcacagca tgctcaagca ccagacgctg gtcctgacgc tgcccaccgt cgccttcctg |
| 11761 | gaggacaagc tgctctggca ggactcacgt tacagacccc tctacccctt cagcctgccc |
| 11821 | tacagcgact tcccccgacc cctMccccac gctacccagg gcccagcggc caccccgtac |
| 11881 | cactgttgaW gtgaagcacc tcttYtgagc caggccgagc ccctggccga cttgggagcc |
| 11941 | tYaggcccac gcccaccctt cgaggaaggt gtcacctgga ccccttcatt ccacggagga |
| 12001 | agctgaggcc acagggagcg gccatcgcca ttgggaaggg gcgactccac ggaRagccca |
| 12061 | gacgggcttY tgcatccatt ccctcttttt gtttttaaaa taaattgtat ttttgaatca |
| 12121 | aggaggataa agataacttc tcagtgtcat ttttgataat tgcattgaga acgatgagct |
| 12181 | ccttcccagg ttctgggcac tgttggggat ccgccgtctc aagaggctca cggtctggtt |
| 12241 | taggggaacc ccagggctgt ttgtggaaat tacaagaatt cacttacccg gtgagtcaga |
| 12301 | ctccaggagc tcacaggtgc gtgggcgctg gcacttccta ggagctgact cctgccacat |
| 12361 | ccctctcctg agcactgctg ccagccattc tcacccctga gagggtttgc agtcctgtct |
| 12421 | gcgacagtac ttcatgcagc ctgagagtgt gtgcttgatt gtacaagtag atgttgtgta |
| 12481 | aacctgagtt ttcagagtga cttcctgtaa gcacagtcag aaaagtaaat gtctctctgt |
| 12541 | aagtgagcag ataagcacat gcttaaaaac accagctggg cgtggtgact cgcacctgta |
| 12601 | accctagcac tttgggaggt caaggtggaa ggatcgtttg agctcagagc ttccagacca |
| 12661 | gcctgggcaa catggtgaaa ccccatctct gcaaaaaata taaaaattaa gtgggcgtgt |
| 12721 | tggcgtccac ctgtggtccc agctacttgg gaggctgagg tgggaggatc acctgagccc |
| 12781 | aggaagcaga ggttgcagtg agctgagact gagccaccac acaccagcct gggtgacaga |
| 12841 | gtgagacact gtcttaaaac cccaatatcc caatttttga gtttgctgag gttgccagcc |
| 12901 | aagttaattt ttcttaacag ccataacaga ctataatata gacaatgtga tcgatttatt |
| 12961 | taaagtcaac cagctgggcg cggtggctca cgcctataat cccagaattt taggaggctg |
| 13021 | agccaggtgg atcaccggaa gtcaggagtt cgagaccagc ctggccaagc cgggagtggt |
| 13081 | ggcgggcacc tgtaatccca gctacttagg aggctgaggt gggagaatcg cttgaaccca |
| 13141 | ggaggcagag gttgcagtga gccaagatag cgccactgcg ctccagcctg ggtgacagag |
| 13201 | caagattcca tctataaata aataaataaa taaagtcaat caggactgcc ttctgcctgt |
| 13261 | gtgggcctgg ccaggctaag cagccacaca accctcccta cctgctgggc cacccctggc |
| 13321 | tgaaaacctc tctggaatgc ctctcttggg attgccttca gagattgcaa cacattctcc |
| 13381 | aaacaccctc agtggacata gactttgatc cttgaagcag cagatttgac taagtaaaaa |
| 13441 | agaaaaagct attaaaacca gttggctggg cgcggtggct cacgcctgta atcccagcac |
| 13501 | tttgggaggc cgaggcaggc ggatcacgag gtcaggagat cgagactatc ctggctaaca |
| 13561 | cagtgaaacc ccatctctac caaaaataca aaaaattagc cgggcgtggt ggtgggcgcc |
| 13621 | tgtagtccca gctactcagg agactgaggc aggagaatgg tgtgaacccg ggaggtggag |
| 13681 | cttgcagtga gccgagatcg cgccactgca ctccactcca gcctggcgac agagctagac |
| 13741 | tgtctcaaaa aaaaaaacag aggcgtgcca aagctcagca gaaaatgccg cctcatcact |
| 13801 | cttcctcttg ccagtcttgt gggggcacca aggcctagag taacacccag ctgttggcct |
| 13861 | gacagtgcct ggcccagcct ggagagttgc agccagaagt ataaatgtgg ttcagtctgc |
| 13921 | gtcatacctg tcaggaaaca tggtgagacc aactgctgcc cagacggatt ttcaaaagaa |
| 13981 | atgggtcaga acgtattccc ccacactgga atccctcagc cagattgagg ataaaaacag |
| 14041 | gcatcagaaa aaaatgatac aggcaggcct ggtggctcat gcctgtaatc ccagcacttt |
| 14101 | gggaggctga ggcgggagga tcgcctgagc acaggagttt gagaccagcc tgggcaacac |
| 14161 | agtgagaacc tatctctact aaaaatagaa caattagcca ggcacggtgg tgtggctgtg |
| 14221 | gtcccagcta ctctggaggc tgaggtggga ggatcacttg agccctaggg gtagaggctg |
| 14281 | cagtgagcgg agatcacccc actgcaatcc agcctgggca acagatcaag accctgtctc |
| 14341 | aaaaaaaaaa aaaagaaaag aaaacaaaag aaaaatatga tacactgact agaatgtgct |
| 14401 | ttaaaataat tggcatagtt gggtatggtg gcatgcacct gcagtctcac ctacttggaa |
| 14461 | agctgtggcc aggagtttga gaccagcctg ggcaacacag caagacctca tctctataaa |
| 14521 | aaataggcgg ggagcagtgg ctcacgcctg taatcccagc actttgggag gccaaggcag |
| 14581 | gcggatcact agaggacagg agttcaagac cagcctggcc aacatggtga aaccccatct |
| 14641 | ctactaaaaa tacaaaaatt ggcaggatgt ggtggcgggt gcctataatc ccagctactt |
| 14701 | gggaggctga ggcaggagaa tcgcttgaac ctgggaggca gaggttgcag tgagccgagg |
| 14761 | tcatgacact gccctcaaac ctgggtgaca gagcaagact cggtctcgaa aaaaaaaata |
| 14821 | atgaatgaat gaattaattc attaattaaa tagggggggt atatgagttt gttagggctg |
| 14881 | ccgtaggagt gccacaaact gcaggggttg ggggggttag tcaacagaaa tttattctgt |
| 14941 | cctgtttctg gaggctggaa gtccaaggtg aagaacaggg ttagctcctt ctaagggaaa |
| 15001 | atctgttcca gatccctctc ctagcgtctg gtggtttgct ttgctgacta tctttgacat |
| 15061 | tccttggctt gtagccacac tgcttcagtc tccaccttca tctttacatg atgttgtccc |
| 15121 | tgtgggtatg tgtctgtttc tgtgtctaaa tttctccttt ttattttttt cttttttctc |
| 15181 | tatctttttt tttagacgga gtcgcgctct gttgcccagg ctggagtgca gtggctcgat |
| 15241 | ctcggctcac tgcaacttct gcctcctggg ttcaagcgat tctcctgcct cagcctccca |
| 15301 | agtagctggg attacaggcg cccaccacca tgcctggcta atttttgtgt ttttagtaga |
| 15361 | gaaggggttt cgccatattg gccaggctgg taacctcagg tgatccaccc gctttggcct |
| 15421 | cccaaagtac tgggattaca agcgtgagcc accgaacctg gcctattttt tcatttattt |
| 15481 | ttagacagag ttttgctctt cttgtccagg ctagagtaca atggcgcaat ctcggctaat |
| 15541 | ctcaacctcc gcctcccggg ttcaggcgat tctcctgcct cagcctccca agtagctggg |
| 15601 | attacaggca tacgccacaa tgcctggcta attctgtttt gttagcagag atggggtttc |
| 15661 | accatgttgg tcaggctggt ctcgaactcc cgacctcagg tgatccaccc acctcagcct |
| 15721 | cccaaagtgc tgagattaca gacacgagcc actgagcctg gccctctcct ttttattttt |
| 15781 | aaaatatatt ttgtggacgg cagcagtggc tcacacctgt aatcccagca ctttgggagg |
| 15841 | ccgaggcggg tggatcacaa ggtcaggaac tcgagaccag cctggccaat atggtgaagc |
| 15901 | cccatctcta ctaaaaatat aaaaattagc caggcatggc cgggcgcggt ggctcacgcc |
| 15961 | tgtaatccca gcactttggg aggccgaggc gggcggatca cgaggtcagg agatcgagac |
| 16021 | catcctggtt aatacggtga aaccccgtct ctactaaaaa tacaaaaaaa aaattagctg |
| 16081 | ggcgaggtgg caggtgcctg tagtcccagc tactcgggag gctgaggcat gagaatggcg |
| 16141 | tgaacccggg aggcggagct tgcagtgagc tgagatcgcg ccactgcact ccagcctggg |
| 16201 | tgacagagtg agactccatc tcaaaaaaaa aaaaaaaaaa tttagccagg cgtggtgtca |
| 16261 | gaagcctgta gtcgcaacta cttgggaggc tgaggcagga gaatcacttg aacccaggag |
| 16321 | gtggaggttg cagtgagccg agaccacatc acggcacatc taaaaaaaaa aaaagttttc |
| 16381 | tgtttttgtt ttttgcaaaa ctactgaaat aaatacagtg agatatttat ttataaatga |
| 16441 | gaacgaatta ataatgagcc gtaggctggg tgtggtggct catgcctata atcccagcat |
| 16501 | tttggggggc caaggcaggt ggaacacttg aggtcaagag ttcgagacca ggctgaccaa |
| 16561 | tatagtggaa ccccatctct actaaaaata caaaagaatt agccgggcat ggtgccgggc |
| 16621 | gcctgtaatc tcagctatgg gaggctgagg taggagaatc gcttaaaccc tggaggcgga |
| 16681 | ggttgcagtg aggcgatatc acgccactgc actccagcct gggggacaga gcgagactcc |
| 16741 | atctctaaat aaataaataa ggagctgtat ttcaaaattt ggagaaggtg acactgagag |
| 16801 | tactgaatac acagtttttt gtttttttgg ttttttttga gacagagtct cgctctgtca |
| 16861 | cccaggctgg agtgcagtgg tgcaacctcg gctcactgca atctctgcct cccggttcaa |
| 16921 | gcaaatctcc tgcctcagcc tcccgcgtag ctgggattac aggcacgcat ccccatgcct |
| 16981 | ggctaatttt tgtattttta gtagagacgg gatttcacca tattggtcag gctgatctca |
| 17041 | aactcctgac ctcgtgatct gcctgcctcg gcctcccaaa gtgctgggat tacaggcgtg |
| 17101 | agccaccgag cctggcccca aaaaatattt atcaaaacta tgttaatgct ggccgggtgc |
| 17161 | ggtggctcat gcctgtaatc ccagcacttt gggaggccga ggcaggtgga acacgaggtc |
| 17221 | aggagattga gaccatcctg gctaacacgg tgaaaccccg tctctactaa aaaatacaaa |
| 17281 | aaattagccg ggcgcggtgg tgggtgcctg tagtcccagc tactcaggag gctgaagcag |
| 17341 | gagaatggca ggaaccgggg aggcagaggt tgtagtgagc tgagatcgcg ccattgcact |
| 17401 | ccagcctggg cgacagagcg agaatccgtc tcgaaaaaaa aaaaaaaata cacacacaca |
| 17461 | cacaaaaact gtgttaatgc ttaactacac aaaaatgata atcagataaa tatgcattta |
| 17521 | tttagagaac tgcatgttgg tcagtccagt ccctgcagag ggaattccca gcatgacctc |
| 17581 | attcacttgt gaagacagag caatccttgt gttttatttt tttaagatgg atctcactct |
| 17641 | gttgcccaga ctggagtgca gtggcatgat ctcagccctc tgccacctcc accttccggg |
| 17701 | ttcaagagat tctcatgcct cagcctcctg agtagctgag attacaggct tgtgcctcca |
| 17761 | tgcccagcta atttttttat ttttactaga gatgaggttt caccagtttg ggcaggccgg |
| 17821 | tctcaaactc ctgacctcaa gtgatccacc cacctcggcc tcccgaagtg ctgggatgac |
| 17881 | aggtgcctgg tcagcaactg ttgtttagac atacacattt tatctgctcg tccagcatgg |
| 17941 | tcagccctcc actttttaaa ttttatttta tttatttttt tgagacagag tctcactctg |
| 18001 | ttgtccaggt tggagtccag tggcgtgatt tcggctcact gcaacctcta cttcccaggt |
| 18061 | tcgagcaatt ctcctgcctc agcttcccga gtagctggga ttacaggccc gcgtccccac |
| 18121 | acctagctaa tttttgtatt tttagtagag acagggtttc accatgttgg ccaggctggt |
| 18181 | ctcgaactcc tgacctcagg tgattctcct gccttggcct cccaaagtgc tgagattaca |
| 18241 | ggtgtgagcc actgcacacg gccttaaatt ttatttatta tttatttatt tatttattta |
| 18301 | gagacttagt ctcactctgt tgcccaggct ggagtgcagt ggcatggtct cggctcactg |
| 18361 | cactccacct cctgggttca cgccattctc ctgcctcagc ctcccgagta gctgggacta |
| 18421 | caggcgccca ccaccactcc cggctaattt ttgtattttt agtagagatg gggtttcact |
| 18481 | gtgttagcca ggatagtctc gatctcctga cctcgtgatc cgcctgcctc ggcctcccaa |
| 18541 | agtgctggga ttacttattt tgttttttgt agagacaggt tctcactgtg ttgcccaggc |
| 18601 | tggtcttgaa ctcctgatct caagtgatct tcccacctca gtctctcaaa gggctgggat |
| 18661 | tacaggggtg agccactgca ccccaccttc cctctacttt ttgacggttt ccttctgcta |
| 18721 | tgaatgtgca tgtccagttg tctgcttctt agaactgata tttaccttcc tcatccatca |
| 18781 | gccattggag gaggactggg accgctcaga ttattgatct gacccattct ttcggcaggg |
| 18841 | tttcctggtg gctgtcttcc atcaccaaaa ctggaatcag aagagtttcc atagcccttt |
| 18901 | ttttttcccc acatctttgc tgaagcagag ttttgaaaaa caaaaccaca aactaagcta |
| 18961 | ttccccagaa gaaatctgta atcaaagata agctctgccg ggcacagtgg ctcacgcctt |
| 19021 | ttggaggcca aggcgggcgg atcacctgag gtcaggagtt ctagacctgc caggccaaca |
| 19081 | tggtaaaacc tcatctctac taaaaataca aaaattagct agatgtggtg gtgggtacct |
| 19141 | gtagtctcag ctacctggga ggctgaggca agagaatcgc ttgaacctgg gaagtagagg |
| 19201 | ttgcagtgag ccgagattgc accactgcac tccagcctgg gcgacggagt gagacgacct |
| 19261 | cacaaaaatt tacataaata aaatgaaaag taaaataaaa atacaaaagt tggccgggtg |
| 19321 | cgtttgctca cgcctgtaat cccagcactt tgggagggtg aggcaggcag ataatgaggt |
| 19381 | aagaagatcg agaccatcct ggctaacacg gtgaaaccct gtctctacta aaaatacaaa |
| 19441 | aaattagctg tgcgtggtga cacgcacctg tagtcccagc tatttgggag gctgaggcag |
| 19501 | gagaatcact tgaacctggg aggtggaggt tgcagtgagc cgagatcgca ccactgcact |
| 19561 | ccagcctggg ccacagagtg agactccatc ttgaaaaaaa aaaaaaatac aaaagttagc |
| 19621 | caggggtgtt ggtgggtgcc tgtaatccca gctatttggg aggctaaggc agaagaattt |
| 19681 | cttgaaccta ggaaacggag gttgcagtga gccgagatca cacctctgta ctccagcctg |
| 19741 | gacaacagag cgagactttg tctcaaaaaa aaaaaaaaaa aaaaaactaa ataggccggg |
| 19801 | agcagtggct catgcctata atcccagccc tttgggaggc caaggcaggt ggatcacttg |
| 19861 | aggtcaggag tttgagacca ggctggccaa catggtgtaa ccccgtctct actaaaaaca |
| 19921 | caaaaattag ccgggtctgg tggcgtatgt ctgtaatccc agctactcgg gaggctgagg |
| 19981 | caggagaatc acttaaacct gggaggcagg ggttgcagtg agctgagatc gtgccactgc |
| 20041 | actctagcca gggtgacaga gtgaaactct gtctcaaaaa attaaaaaag aaattcagca |
| 20101 | agtaatgagt taaggaattc gaatattaag gcgagtgaca aggaacgccc aggatgtggc |
| 20161 | ccaggatgga gtagggggga cactcattta ggagaaagct caggccacaa gacaggagga |
| 20221 | gccagccttg ttggggttga agggaagagc attccaggct gagggaactg caaggcgttt |
| 20281 | gcatgggaca ctatgggatg gcttctgccc ttggtgggca gcctctggtc tgaggccatt |
| 20341 | ctttggcctg cctgactgtc tggcaaccgg gaggaagccc tgcccttcct ggagacagaa |
| 20401 | acaaaggtct aggaaatatc tgcttccctt ttccttgaaa aacgcttaag ggaacggagg |
| 20461 | actgggaggt gccgtctctc tctgccagcc tgccccctac catagccatc ccactcccat |
| 20521 | ctcagaaagt gacccgccat cctccaaaag gctcggaccc tgatcaagga gtcatccccc |
| 20581 | ttgtcccagc acctccagtt ggcccagcct ccaaaacgga tgtcaaattc agccctttct |
| 20641 | ccaaggacac tgcccagtcc aggccccact atcattcatc tggactagaa cagtcacctc |
| 20701 | ctctcccatc tcctggctgc agctcttgaa gcctcaactg ggcccctgtg aacacttgag |
| 20761 | ttagggcaag gtccttcctc tgctcagaac cctctatacc tcccacctcg ctgggcataa |
| 20821 | aagccaaagt cctggccagg cacggtggct cacatctgtt atcccagcac tttgggaggc |
| 20881 | caaggggggc ggatcactag aggtcaggag ttagagacca acatggtgaa accccatctc |
| 20941 | tactaaaaat acaaaaatta gctaggcgtg gtgacgcacc cctgtagtac cagctactcg |
| 21001 | gtaggctgag gtgggagaat cgcttgaacc tgggaggcag agtttgcagt gagccgagat |
| 21061 | cacaccactg tgctccagcc tgggtgacag aacgagactg gggttcagaa acaaacaaac |
| 21121 | aaaacaacaa agtcctcctc aggtgacagg aacttgcacc tatctgccct gtcatctccc |
| 21181 | tgcccgctcc tctcctcgaa tctctccttt gctaagcctg ctccagccac actgttctcc |
| 21241 | tggctgttcc tttttttttt tttttttttt tttttttgag tctcactctc acccaggctg |
| 21301 | gagtgcagtg cctctatctt ggctcactgc aacctccgcc tgccgggttc aagagattct |
| 21361 | cctgcatcag cctcccaagt aggtggaatt acaggtgtgc accaccacac ccggctaatt |
| 21421 | tttgtatttt gcatagagat gggggtctcc ctatgttgcc caggctggtc ttgaactcct |
| 21481 | gggctcaagt gatcctScca tctcggcctc ccaaaatgct gggattacag gtgggaRccg |
| 21541 | cgcccaggtg gatttttgtc tgactctgtt cattcctgtg tccccagtac ctggaaggac |
| 21601 | gccaagcaca cagtaggcgc ttaaaaaaca ttgagccaca tgttgagaaa agaacggcac |
| 21661 | cattgtggct gcaagtggga cKtgggccgc gcgggggagc tcgcgcacct cgggccgggg |
| 21721 | caagagctca gtggaacccg cccgaggaag aacccgtggc gcaggatttt cccaggcctt |
| 21781 | ctgaggacca ggggcgtccc ccgtcccacc ctgtgacttt gctcaggccg ttccggggcg |
| 21841 | ggaattcaga actcctcagc cccccaagaa aaaaatatcc ccgtggaaat tccttgggaa |
| 21901 | tgaccgaggc gggggaaata tgcgtctctg gatggccagt gactcgcagc ccccttcccc |
| 21961 | gataggaagg gcctgcgcgt ccggggaccc ttcgcttccc cttctgctgc gcgacctccc |
| 22021 | tggcccctcg gagatctcca tggcgacgcc gcgcgcgccc cacaacagga aaRccttagg |
| 22081 | cggcgcggct tggtgctcgg agacttaaga gtacccagcc tcgacgtggt ggatgtcgag |
| 22141 | tcttggggtc acacgcacag gcggtggcca agcaaacacc cgctcatatt tagtgcatga |
| 22201 | gcctgggttc gagttgccgg agcctcgcgc gtagggcagg ggttcgagcg ccccttctcc |
| 22261 | ctgcctcgcc tctgcgcctg ggggctgctg cctcagtttc ccagcgacag gcagggattt |
| 22321 | cgagcgtccc cctcccctcc ctcgtcaaga tccaagctag ctgcctcagt ttccccgcgg |
| 22381 | agcctgggac gcNNgcggag gggctcggcg cgtagggatc acgcagcttc cttccttttt |
| 22441 | ctgggagctg taaagacgcc tccgcggcca aggccgaaag gggaagcgag gaggccgccg |
| 22501 | gggtgagtgc cctcgggtgt agagagagga cgccgatttc cccggacgtg gtgagaccgc |
| 22561 | gcttcgtcac tcccacggtt agcggtcgcc gggaggtgcc tggctctgct ctggccgctt |
| 22621 | ctcgagaaat gcccgtgtca gctaggtgtg gacgtgacct agggggaggg gcatccctca |
| 22681 | gtggagggag cccggggagg attcctgggc ccccacccag gcagggggct catccactcg |
| 22741 | attaaagagg cctgcgtaag ctggagaggg aggacttgag ttcggacccc ctcgcagcct |
| 22801 | ggagtctcag tttaccgctt tgtgaaatgg acacaataac agtctccact ctccggggaa |
| 22861 | gttggcagta tttaaaagta cttaataaac cgcttagcgc ggtgtagacc gtgattcaag |
| 22921 | cttagcctgg ccgggaaacg ggaggcgtgg aggccgggag cagcccccgg ggtcatcgcc |
| 22981 | ctgccaccgc cgcccgattg ctttagcttg gaaattccgg agctgaagcg gccagcgagg |
| 23041 | gaggatgacc ctctcggccc gggcaccctg tcagtccgga aataactgca gcatttgttc |
| 23101 | cggaggggaa ggcgcgaggt ttccgggaaa gcagcaccgc cccttggccc ccaggtggct |
| 23161 | agcgctataa aggatcacgc gccccagtcg acgctgagct cctctgctac tcagagttgc |
| 23221 | aacctcMgcc tcgctatggc tcccagcagc ccccggcccg cgctgcccgc actcctggtc |
| 23281 | ctgctcgggg ctctgttccc aggtgagtcg gggtggggat tgccgtcggg ccagttctcc |
| 23341 | gaagccccgg gaggaccggc tcccgggtca ggtcatgcat gcttaggtag ctgtttatgg |
| 23401 | gaaggagggg ctagagacag cgattgaaag gcaacagcca gtaggttcga atccagaccc |
| 23461 | tgcatacctc cacgtgtggc cttgggctat agattgcagc tttaaaaaag ggtagggggt |
| 23521 | tggagatgga ggggaggggc gggcctcgtt ttgttgccca ggccggtctt gaactccggg |
| 23581 | ggtctagcct tacctcctgc ctcagcctcc cgaStagctg ggatgagagg tgtgaaccac |
| 23641 | cgccttgctt ggctagattg cgtctcttac agtttctcag Ytgtaaaacg ggaaacgtta |
| 23701 | tagcggccac ctggcagggt atcttggccc agcgcagcac ctggccccag gactcgatca |
| 23761 | tgatggtttg ggaacttggc tctgtgccaa cccaacaagg cttaagggac ccccaccccc |
| 23821 | ctcaagatgt atattctgtt cctcatcctc tctgcccctg gggaagtcca gggctgcttc |
| 23881 | tacttggggg aattccagag ctgacttatc cgtggcccaa agctgagaag tgggacRccc |
| 23941 | cagcacaccc tcccccagct ccagcccagc tagggaagag ggaaggggtc agagggtctt |
| 24001 | tcatggtggt gtaagtttgg ggaaccagga gggtggRaga ttgacagctt ggttaacagc |
| 24061 | tcaacaaagc ctgagatcca ggccagcacg gtagttcatg ccagtaatcc caacacttta |
| 24121 | ggagccccag gcgggcgaat cacttaaggt caggagtttg agaccagcct ggccaacatg |
| 24181 | gcaacatccc gtctctacta aaaatacaaa aattagctgg catggtggtg ggcgcctgtg |
| 24241 | atcccagctg ctcgggaggc tgagggagga aaatccctta agcccacgag gctgaggttg |
| 24301 | cagtgaacca agattgtgcc actgcactcc agcctgggag acatagcgag attctgtctc |
| 24361 | aaaaaacaaa gcRttctgat ccggactcag acccagatcg cactgctttc tagctgagta |
| 24421 | accatttctc tctatgaaat gggaatggtc ccagaatctc ccttggagaa tgtatggagc |
| 24481 | cagtgtcctc acacccccat ccaagataga acaaatctga gacaggaatc tttgagtgag |
| 24541 | gcagtgctgg gctcagacat tttttcccac cttcggaggc agcagaatYt gagggacctg |
| 24601 | atccaaataa gccccttctt tctttctttt cttttctttt tttttttttt ttttttttga |
| 24661 | gacggagtct cactNNgtcK cccaggctgg agtgcagtgg cgtgatctcg gctcactgca |
| 24721 | acctctgcct cccaggttca aacgattctc ctgYctcagc ctccctgagt agctgggact |
| 24781 | acaggcatgt gccatcacac ccggctaatc actgtgttag ccaggatggt ctcgatttcc |
| 24841 | tgacctcatg atctgcccac cctgcctccc aaagtgctgg gattacatgc gtgagccaca |
| 24901 | gtgcccaccc cgtaagcccc ttctttctta cctgcaaggt agccagttgc tacccatcct |
| 24961 | gtgctgagtt acttgtatta gcaagggatg gggtggctat actcacccac cttacagatg |
| 25021 | gggaaattga ggcccaaaga gggggaaact acRtgtctca gggagtgagg agccagtctg |
| 25081 | attcctggag ggctgactgt ctccacctga cttcttagga gggaggaggg caccaacttc |
| 25141 | acattaaaat ctggttggac acagtggctc acacctgtaa tcctggcatt ttgggaggct |
| 25201 | taggcgggag gatcacttga ggccaggagt ttgagaccag ccttagcaac atagtgagac |
| 25261 | cccatctcta caaaaatgtt tttcagggcc aggcgcggtg gctcacacct ataatcccag |
| 25321 | cactttggga ggctgaggcg ggcggattac ctgaggtcag gagtttgaga ccagcctgac |
| 25381 | caacatggag aaaccccgtc tctactaaaa gtacaaaatt acccgggcgt ggtggcgcat |
| 25441 | gcctgtaatc ccagctactc gggaggctga ggcaggagaa tcgcttgaac ctgggaggcg |
| 25501 | gaggttgcgg tgaactgaga tcgtgccatt gcactccagc ctgggcaaca agagctaaac |
| 25561 | tccgtctcaa aaaaaaaaaa atgtttttca aatattagcc gggtatggtg gtgtcctgta |
| 25621 | gtcccagcta cttgggaggc tgagatggga ggatcacttg agcccaggag ttcaaggtta |
| 25681 | cagtgagcta tgattgtgcc actgtattcc agcctgggta acagagggag acccgtttaa |
| 25741 | aaaaaaaaaa gtgatggcta aagtccttcc atggctccct attgccctca gtataaagaa |
| 25801 | cacatgtggc tgggcgtggt ggttcacgcc tgtaatccca gcactttggg aggctgaggc |
| 25861 | gggcggatca cttgaggcca ggagtttgag agcaggctgg ccgacgtggc gaaaccccgt |
| 25921 | ctctattaaa aatacaaaaa ttagctgggc gtggtggtgc ttgcctgtaa tcccagatac |
| 25981 | tctggaggct gaggcaggag aatMacttga acccgggagg caRaggttgc agtgagctga |
| 26041 | gattgYgcca ctgcactcca gtctgagtga caaagcgaga ctccatctca aaaaaaaaaW |
| 26101 | aaataaaaga acacatcttt agcatggcct tcagtgctca cgggatcttc ctgaattaat |
| 26161 | ctccccctct tcatccttgc tcactcagct ccagccaccc tgccccggga catctgtact |
| 26221 | tgcctggaac ttatttccct tttctccgga cagccagccc tttctcgtca tttagatctc |
| 26281 | tgctgaaaca ttaccctgtc accaaagcac tgtctattct atcaccctgt tttgtttttg |
| 26341 | tcaaagctca tattaacatc agttattaat tatcttgttt gctcataatt tttttttttt |
| 26401 | tttttttgga gacagagtct cgttctgttg ctcaggctgg agtgcagtgg cacaatcttg |
| 26461 | gctcactgta acctccacct cccaggttca agtgattctt gtgcctcagc ctcccaaata |
| 26521 | gctaggacta caggcacgtc ccaccatgcc cagctaattt ttgtattttt agtggagacg |
| 26581 | gggctttgtc atgttggcca ggctgatctc aaattcctga cctcaagtga tctgcccgcc |
| 26641 | ttggcctccc aaagtgctgg gattacaggc gtgagccacc acacccggcc tgctcatgaa |
| 26701 | ttttctcttt aacttccaca tcgaaggcaa agtattgtct tgttaaggct gtgcctccag |
| 26761 | cacccagcac aggctgggcg cacattccct tgatgaacct gatttgtaat gcctgtYgcc |
| 26821 | tcttccctcg tttcttctag gacctggcaa tgcccagaca tctgtgtccc cctcaaaagt |
| 26881 | catcctgccc cggggaggct ccgtgctggt gacatgcagc acctcctgtg accagcccaW |
| 26941 | gttgttgggc atagagaccc cgttgcctaa aaaggagttg ctcctgcctg ggaacaaccg |
| 27001 | gaaggtgtat gaactgagca atgtgcaaga agatagccaa ccaatgtgct attcaaactg |
| 27061 | ccctgatggg cagtcaacag ctaaaacctt cctcaccgtg tactgtgagt aactgagccc |
| 27121 | ggagggctgg actaggcaga ccSggtggga gagacgtgca gRggcacctg cagaggcctg |
| 27181 | ggggaatctt tgccacttgc tcgtagggtc aaggaggggc tccttgcagg gcaggtgggg |
| 27241 | acatccttgg aaagtccctt tgtgaatttc tttgggtaca attaaagtat ttacaggctg |
| 27301 | ggtgcggtgg ctcatgcctg taatcccagc actttgagag gctgaggctg gcggatcacc |
| 27361 | tgagatcagg agtttaagtt tcgccaacat ggcgaaaccc tgtctctgct gaaaatacaa |
| 27421 | aaatcagccg ggcatggtgt caagcgcctg taatcccagc tacttggaag gctgaggcag |
| 27481 | gagaacgctt gaacctgaga ggcagagatt gcagtgagcc gcgatcgtgc cagtgcactc |
| 27541 | cagtctggat aacagagcaa gattccatct caagaaaaaa aaaatgccat ctctctatgc |
| 27601 | ctcactcttt gaacatatga cacggtcctg cttcagacac tttaataaaa gatgcaaatt |
| 27661 | aagccaagtg tggtggcttg tacctataat cccaactact ccagaggctg aggcagaagg |
| 27721 | atggtttgag cccaggagtt tgagaccagc ctgggcaaca gagtgagacc ctgtttcttt |
| 27781 | cttttttttt tttttttttt tgagacggag tctcactctg tcgcccaggc tggagtgcag |
| 27841 | tggtgtgatc tcggctcact gcaagctccg cctcccgggt tcacgccatt ctcctgcctc |
| 27901 | agcctcccga gtagctggga ctacaggcgc ctgccaccat gcccggctca tttttttgta |
| 27961 | tttttagtag agacggggtt tcactgtgtt agccaggatg gtctcaatct cctgacctag |
| 28021 | tggtccgccc gcctcggcct cccaaagtgc tgggattaca ggtgtaagcc actgtgccca |
| 28081 | tccaagaccc tgtttctacc ggaaaaaaaa agtaaataat ttagctgggc atcgtggtgt |
| 28141 | gcacctgtaa tcccagctgc tcctgaggct gtgatgggag gattgcttta acccaggggt |
| 28201 | tcgaatccta ggagttcgaa tccatcctag gcaacatagc aaaaccccat ttttatttaa |
| 28261 | aaaaaaaaaa aaagatatga gttaaaacta gccctgggat ggcatttttc acatattggt |
| 28321 | aacaaacRaa agaattgatg gccgggcgca gtggctcacg cctgtaatcc cagcactttg |
| 28381 | ggaggccgag gcgggcggat cacaaggtca ggagatcgag accatcctgg ctaacatggt |
| 28441 | gaaaccccgt ctctactaaa actacaaaaa attagccggg catggtggca ggcgcctgtg |
| 28501 | gtcccagcta ctcaggaggc tgaggcagga gaatggcatg aacccgggag gcagagcttg |
| 28561 | cagtgagcca agatcgtgcc actgcactcc agcctgggcg acagagcaag actccatctc |
| 28621 | aaaaaaaaaa aaaaaaaaaa gaattgataa cagctgtgct gccaaggcta ttggaacgta |
| 28681 | ggaggtccta ggacagtgct gttgggagca taaataagcc caaccctgtg gcgggaaatt |
| 28741 | gggcatcagt tctcaaaatg tcatgggctg ggcacggtgg ctcacgcctg taatcccaSc |
| 28801 | actttgggag gctgagggag gcggatcact tgaggtcagg agttcgagac cagcctgacc |
| 28861 | accatggaga aaccccgtct ctattaaaaa tacaaaaaaa actagccagg catagtggca |
| 28921 | catacctgta atcccagcta ctcgggaggc tgaggcagga gaatctcttg aaactgggag |
| 28981 | gcagaggttg cggtgagctg agattgcgcc actgcactcc agcttgggca acaagagcaa |
| 29041 | aactccatct caaaaaaaaa aaaaaaagaa ataaaagaag gtatgttgaa tatgagtggt |
| 29101 | atgccaccct cacattaggg aagggcagtt tcggggaggc tgtatttatg tataaaatag |
| 29161 | ccctaMaagg aagtgggaga aatgacaata ttagctggct atgagaagag aggctgggag |
| 29221 | gctgtgggag agggcttggg tgtggagaat tctttttgtt ttttcctttt tttgagacag |
| 29281 | agtttcactc ttgttgccca ggctggagtg caatggcacg atctcagctc Mccgcaacct |
| 29341 | tcacctcctg agtttaagtg attctccggc ctcagcctcc cgactagctg ggattacagg |
| 29401 | catgggccac tacgcctggc aaattttgta tttttagtag agacagggtt tctccatgtt |
| 29461 | ggtcaggctg gtcttgaact ctgacctcag gtgatccgcc cgcctcggcc tcccaaagtg |
| 29521 | ctgggattac aacgtgagcc actttgcctg gctgagaatt ctttttttgt tgttgtcttt |
| 29581 | ttgagatgga gttttgctgt gtccccagcc tggagtgcaa tggtgtaatc tcagctcact |
| 29641 | gcaacctctc cctcccgggt tcaagcaatt ctcctgcctc agcctcccaa gtagctggaa |
| 29701 | ttacaggcgc ccagcaccac gccYggctaa tttttgtatt tttagtagag acgggatttc |
| 29761 | atcatgttgg ccRggctggt cttgatctcc tgaccttgtg atctgcccgc ctcggcctcc |
| 29821 | caaagtgctg ggattacagg cgtgagccac tgcccccagc cRagaatttc tctttgYgtc |
| 29881 | cttcctactt tggggacttc gaatggtggg aaagagttat caaggccaaa ataaggaatt |
| 29941 | caaatgaaaa caaaacaaaa tcaaagaaga aaaaacagaa gagcactggg caggctaggc |
| 30001 | acgtggctca tgcctataat cccagtgatt tagaaggccg aagtagaagg atcgcttgga |
| 30061 | ggccaggagt tggacaccag cctgggcaac atagcaagac cccatatcta caaaaaataa |
| 30121 | aaaacctaac caagcgtgct ggcatactag tagtcccagc tactcaggag gctgaggtgg |
| 30181 | gaggatcacc tgaKcctggg aggtccaggc tgtagtgagc cgtgatgaca ccactgcact |
| 30241 | ccagcctggg tgacagaKaa agaccctgtc tctaaaaaat aaaaactggc caagtagctt |
| 30301 | tgggattagc cttgggttcc agtcccagca aggcctttaa tagcttggga catgacttct |
| 30361 | gcatttactt tgcaatcagg tgagacctcc tctgatgggg aaaatgacac ggtgagtgac |
| 30421 | aaaggatgtt ctcctatcat tgtgtcaggg caaggaagcc tctgggtaaa tgatcaaatg |
| 30481 | atcagctttg cttctgattt ggagggtggg tgagcagatg ctgaccttcc caggtgaggg |
| 30541 | aagtccccRa acattcccag cagcttctgg aaaccccagg gaaacctctt tgaaggtctt |
| 30601 | ttctgcatct ctgcctgata ggtctttttt tttttttttt tttttttYtt ttttttgaca |
| 30661 | cacagtcttg ctctgtcgcc caggctggat cactgcaacc tccatctccc gggttcaagc |
| 30721 | agttctcctg cctcatcctc tccagtagct gggattacag gcacctggca ccacgcctgg |
| 30781 | ctaatttttg tatttttagt agagacaggg tttcgccaag ttggccaggc tggtctcgaa |
| 30841 | ctctagacct ctggtgatcc acccgcctca gcctcccgaa gtgctgggat tacaggcttg |
| 30901 | agccaccaYg cgcggctctg cctgatagct gagagcatag aactccaggt ttgagacctg |
| 30961 | gMtctgccac atttctccct ctatgactgt gggtgcccca ctttgcctta gtttttacct |
| 31021 | ctgtgaaatg gagcagatgg ctggcacagg tagcaaagga gtaaaagtta tgtgggaggg |
| 31081 | tggtacctga gagagactct agcttggtct tgccccaccc ctggtgtaaa cataaagaag |
| 31141 | cctccctgga tggctcaatc ttctccaaaa aggttagagg tgtaattcct agaggaggcg |
| 31201 | accactagct gggctttgaa ggatgtgtag gagttcataa ggacaggcat tctgggcagg |
| 31261 | aggaacagcc tgggcaaaag ttgggagcag ggagaaatct tgatggaggc aggaggagga |
| 31321 | ggaggtaggt tggtgtRggc caggYgcagt ggctcacacc tgtaatccca gtgctttggg |
| 31381 | aggccaaagc aagaggattg tttgagccca ggagttcgag accagcctgg gcaacatagc |
| 31441 | gagaccctgt ctctaagaaa aaataaaaaa attagggtac agtggcatat gcttgtattc |
| 31501 | tcaactactc tggaggctca agtgggagga tcaYttgaac ccaggaattt ttttgttttt |
| 31561 | gtttttgttt ttttgagatg gagtctcact ctgtggccca ggctggagtg cagtggtgcc |
| 31621 | atcttggctc actgcagcct cctccacctc ctgggttcaa ccgattctgc tgcctgagcc |
| 31681 | tcccgagttg ctgggattaa ggtgcccacc atcatgccca gctaattttt ttgtattttt |
| 31741 | agtagagata gggtttacca tgttagccag gctggtcttg aacgcctgac cgcaagagat |
| 31801 | cctcctgcct cagtttccca aagtgctggg attataggtg tgagccactg agcctggtca |
| 31861 | agcccaggaa tttgaggtta cagtcagcta tgattgcacc actgcattcc agcccaggtg |
| 31921 | acagagagag acactgcccc tNaaaaaaaa aaaaaaattg attgatggga ggaagggtga |
| 31981 | ggttggcaga gccttgaatg ccaggtggag gagctgggac tttccttctt ggggtgatag |
| 32041 | ggagtcatgg agggtttStg agcaggccag ggattagata gctgaaggct ggatttactg |
| 32101 | gaagccaaWg agcagttggc tatggtcctt gtccacgcgg cccatgttgt gggcagtgac |
| 32161 | cgtattcaag aagggaagga cagacaagta tttgaatact tcagtgacca ggatttggta |
| 32221 | aaggactgca ggtcagggtc aagaagaggt gagagcagga cagacttcct ccccgctgca |
| 32281 | ccaggcagct gagctgggtt tcctctaggg gctgaggttt gagggtacct caagttctgc |
| 32341 | aagagtctat aggaggtggt aagagagaag agctggaggt cagagttttc ttgactatat |
| 32401 | Rtatatatat ttttttgttt ttgtttttaa cagcttaaca gctttctgtt ttatttttag |
| 32461 | agacagggtc tcagggtctc actttgtcac ccaggctgga gtgcagtggt acaatcgtag |
| 32521 | ctgactgcag cctcaaactc ccaggctcaa gaaatcctcc tcccaccctt agcctcctga |
| 32581 | gtagcaggga ctacaggtgt gagccagcag gaagcccagc tggttttttt tttttctttg |
| 32641 | gtgttttttg tttgtttgtt tgagaccgga gtttcgctct tattgcccag gctgaagtgc |
| 32701 | aatggcagga tcttagctca ccacaacctc cgacccccag gatcaagcta ttctcttgcc |
| 32761 | tcagccacct gagtagctgg gattacaggc atgcgacacc acacaaggct aattttgtat |
| 32821 | ttttagtaaa gacagggttt ctccRtgttg gtcaggctgg tctYgaactc ccaacctcag |
| 32881 | atgatccacc tgcctcggcc tcccaaagtg ctgagattac aggcatgagc caccgtgccc |
| 32941 | ggcctttttt tttttttttt ttttNNtgag acagagtctc actctgtcgc ccaggcagga |
| 33001 | gtgcagtggt Kcgatctggg ctcactgcaa gctccgcctc ccgggttcac tccatcctcc |
| 33061 | tgccttagcc tcctgagtag ctgggactac aggcgcccac caccacgcct ggctaatttt |
| 33121 | ttgtattttt agtagagacg gggtttcacc gtgttagcca ggatggtctY gatctcctga |
| 33181 | cctcgtgatc cgcccgcctt ggcctcccaa agtgctggga ttacaggcgt gagccaccat |
| 33241 | gtctggcctg gccaggctgg tcttgaactc ctgacttccg gtgatccatc tgttctggcc |
| 33301 | tcccaaagtg ctgggattac aggcataagc caccacgcca tgccgaagcc cagcttgttt |
| 33361 | ttaatttttt tttttttttt tggagaaatg aggtcttgca atgttgccca agctagcctt |
| 33421 | gaactcctgg cctcaaatga ccccgccttg gcatcccaaa gtactgggat tacagatgtg |
| 33481 | agccaccatg ccccagcctt gctttcttga gatacRattt agaataccat aagattcatc |
| 33541 | ccttttaagc acataattca atgacttctg tacaaacaac catgactaca atctaatttt |
| 33601 | aaaatatYtc aatcactcta aaMaagaaac ctcctgctta tgtacagcga ctctgtctac |
| 33661 | ctcttaagtg aMttctccta cctttaatag ccctatttta cagttcagga aactgaggtt |
| 33721 | cagNgagaca aagtcactta cccacagcaa agaagcaagg ctgggtatca aatgcaggac |
| 33781 | ccccccKgtc ctgatgcttt tttttttttt ttttttttcc tctgagagag actctcactc |
| 33841 | tgtcactcag tctagagtac agtggcgcga tctcagttca ctgcaatctc tgcctcctgg |
| 33901 | gctgaagtaa tcctttcctc acaagtaaac ctcagcctct caagtagctg ggactacagg |
| 33961 | cacacaacac cacgcctggc taatttttgt atttttaggt agagacgggg tttcactatR |
| 34021 | ttggccaggc tggtcttSaa ctcctgacct caggtgatcc gcctgcctcg gccccccaaa |
| 34081 | gtgttgggat tacaggcgtg agccaccaca cgcagccttt ttgttattag actctgtcat |
| 34141 | tactgacttt tttttttttt taatagaaac agggtctttc tttcccaggS taaagtacag |
| 34201 | tggcatgatc acagttcact atagccttaa actcctgggc tcaagtgatc ctcctgcctc |
| 34261 | agcctcccaa gtagcaggga ctacaggtgt gcaccaccac acccagttaa ccattcattc |
| 34321 | attcattcat tcatttattt tgagatggag tctcgctctg tcacctaggc tggagtgcag |
| 34381 | tggcacgatc tcagctcact gcaacctcca cctcccaggt tcaagagatt ctcctgcctc |
| 34441 | agcctcccga gtagctgaga ctacaggcgt gcaccaccat gccagactaa tttttgtatt |
| 34501 | tttaatagag acggggtttc actctgttgg tcaggcttat ctcgaactcc tgacttcgtg |
| 34561 | gatccaccct ccttggcctc ccaaagtgct gggattaaag gcgtgagcca ccgcgccctg |
| 34621 | ccaacctttt tttaattttt cttagagatg ggggtctccc tatgttgccc aggcttgtct |
| 34681 | tgaactcctg gcctcaagtg accctSttgc cttggcctca caaagtgcta ggattacagc |
| 34741 | ctgagccatc acacctggcc aacaggtttt tttttttKtt ttgttttgtt ttttaaagaa |
| 34801 | tgtctaggcc aggctcattt actttcacct gtaatcccag cactttggga ggccggggtg |
| 34861 | ggcagatcac ttgaggtcag gaattcgaga ccagcctggg caacatgctg aaaccccgtt |
| 34921 | tctactaaaa atacaaaaat tagctgggtg tggtgacacg tgcctgtaat cccagctact |
| 34981 | caggaggttg aggcaggaga attgcttgaa cccaggaggc agaggttgca gtgagccaag |
| 35041 | atcatgccat caccctccag cctgggcgac agaaggagac tcagtctaaa aacttaatta |
| 35101 | attaattaat taaaaataaa aatacaaaaa ttaacctggt gtggtggtgt gtgcctgtaa |
| 35161 | tctaagctac tcaggaggct gaggcaggag aatcccctga atcccagagg cagaggttgc |
| 35221 | agtgagccaa gatcgagcca ctgtttgccc agtctagtgc actgggctgc tgaatttatt |
| 35281 | tgaccagaca cctagcaata gactttgaag ttcttttcca cttttcactc taagatgctg |
| 35341 | ctgtcatgaa taaggaatat tttgatcccc ttcacaaaca ctcggggccc tcttaccagt |
| 35401 | tttcactgaa gatcttgaca ttcctatctg cttaggtgtc tgggcgtgtt tgggggagat |
| 35461 | actgaagagg tagggctccc aggcaggtgc agttcgtctg ttaggcaggc agcaaggtcc |
| 35521 | acttcaccag acacccccac ctctgttttc ctRcagggac tccagaacgg gtggaactgg |
| 35581 | cacccctccc ctcttggcag ccagtgggca agaaccttac cctacgctgc caggtggagg |
| 35641 | gtggggcacc ccgggccaac ctcaccgtgg tgctgctccg tggggagaaS gagctgaaac |
| 35701 | gggagccagc tgtgggggag cccgctgagg tcacgaccac ggtgctggtg aggagagatc |
| 35761 | accatggagc caatttctcg tgccgcactg aactggacct gcggccccaa gggctggagc |
| 35821 | tgtttgagaa cacctcggcc ccctaccagc tccagacctt tggtgaggat tgaagaagcc |
| 35881 | agcagggaga aggtgggggt ggggtatcct gcaatgcggt gcctgtggcc acaggatctt |
| 35941 | ttgagatggg tgtggccccg gctaaggggt gcatgtgttc taggcgtatg tgacctaggc |
| 36001 | tgctgagtgg ccctggaaga ggatctcgca ggagggggaa tgaaatgccc cagagaaggg |
| 36061 | cttcgggacg tccatccctg tctgctcaca cctttcttct ctccctagtc ctgccagcga |
| 36121 | ctcccccaca acttgtcagc ccccgggtcc tagaggtgga cacgcagggg acMgtggtct |
| 36181 | gttccctgga cRgKctgttc ccagtctcgg aggcccaggt ccacctggca ctgggggacc |
| 36241 | agaggttgaa ccccacagtc acctatggca acgactcctt ctcggccaag gcctcagtca |
| 36301 | gtgtgacYgc agaggaYgag ggcacccagc ggctgacgtg tgcagtaata ctggggaacc |
| 36361 | agagccagga gacactgcag acagtgacca tctacagtaa gaaggggcag gggcggagtg |
| 36421 | gggcttcttg Rgggtgtgac ctgaacccgg ggcggggctc actgtgtgcc tattccaggc |
| 36481 | tttccggcgc ccaacRtgat tctgacgaag ccagaggtct cagaagggac cgaggtgaca |
| 36541 | gtgaagtgtg aggcccaccc tagagccaag gtgacgctga atggggttcc agcccagcca |
| 36601 | ctgggccYga gggcccagct cctgctgaag gccaccccag aggacaacgg gcgcagcttc |
| 36661 | tcctgctctg caaccctgga ggtggccggc cagcttatac acaagaaYca gacccgggag |
| 36721 | cttcgtgtcc tgtgtgagtg gggctgctgg tcaatggccc ctatccccca aggcccaatc |
| 36781 | tccctgaagg tcccataagg tcttgcctcc aagtcctgcc cccacccacc tccatgtcat |
| 36841 | ctcatcRtgt ttttccagat ggcccccRac tggacgagag ggattgtccg ggaaactgga |
| 36901 | cgtggccaga aaattcccag cagactccaa tgtgccaggc ttgggggaac ccattgcccg |
| 36961 | agctcaagtg tctaaaggat ggcactttcc cactgcccat cggggaatca gtgactgtca |
| 37021 | ctcgagatct tgagggcacc tacctctgtc gggccaggag cactcaaggg gaggtcaccc |
| 37081 | gcRaggtgac cgtgaatgtg ctctgtgagt gagccggcgg gcagagctgg gtgggggcag |
| 37141 | gggccatgga cctaatgcaa tcctcaccgc ctgttgtatc ctccccacag cccccYggta |
| 37201 | tgagattgtc atcatcactg tggtagcagc cgcagtcata atgggcactg caggcctcag |
| 37261 | cacgtacctc tataaccgcc agcggaagat caagaaatac agactacaac aggcccaaaa |
| 37321 | agggaccccc atgaaaccga acacacaagc cacgcctccc tgaacctatc ccRggacagg |
| 37381 | gcctcttcct cggccttccc atattggtgg cagtggtgcc acactgaaca gagtggaaga |
| 37441 | catatgccat gcagctacac ctacYggccc tgggacgccg gaggacaggg cattgtcctc |
| 37501 | agtcagatac aacagcattt ggggccatgg tacctgcaca cctaaaacac taggccacgc |
| 37561 | atctgatctg tagtcacatg actaagccaa gaggaaggag caagactcaa gacatgattg |
| 37621 | atggatgtta aagtctagcc tgatgagagg ggaagtggtg ggggagacat agccccacca |
| 37681 | tgaggacata caactgggaa atactgaaac ttgctgccta ttgggtatgc tgaggYccca |
| 37741 | cagacttaca gaagaagtgg ccctccatag acatgtgtag catcaaaaca caaaggccca |
| 37801 | cacttcctga cggatgccag cttgggcact gctgtctact gaccccaacc cttgatgata |
| 37861 | tgtatttatt catttgttat tttaccagct atttattgag tgtcttttat gtaggctaaa |
| 37921 | tgaacatagg tctctggcct cacggagctc ccagtcctRa tcacattcaa ggtcaccagg |
| 37981 | tacagttgta caggttgtac actgcaggag agtgcctggc aaaaagatca Ratggggctg |
| 38041 | ggacttctca ttggccaacc tgcctttccc cagaaggagt gatttttcta Ycggcacaaa |
| 38101 | agcactatat ggactggtaa tggttacagg ttcagagatt acccagtgag gccttattcc |
| 38161 | tcccttcccc ccaaaactga cacctttgtt agccacctcc ccacccacat acatttctgc |
| 38221 | cagtgttcac aatgacactc agYggtcatg tctggacatg agtgcccagg gaatatgccc |
| 38281 | aagctatgcc ttgtcctctt gtcctgtttg catttcactg ggagcttgca ctatgcagct |
| 38341 | ccagtttcct gcagtgatca gggtcctgca agcagtgggg aagggggcca aggtattgga |
| 38401 | ggactccctc ccagctttgg aagcctcatc cgcgtgtgtg tgtgtgtgta tgtgtagaca |
| 38461 | agctctcgct ctgtcaccca ggctggagtg cagtggtgca atcatggttc actgcagtct |
| 38521 | tgaccttttg Rgctcaagtg atcctcccac ctcagcctcc tgagtagctg ggaccatagg |
| 38581 | ctcacaacac cacacctggc aaatttgatt tttttttttt ttYcagagac ggggtctYgc |
| 38641 | aacattgccc agacttcctt tgtgttagtt aataaagctt tctcaactgc ctcagccttg |
| 38701 | tgtgagttga ggggaggtgt cacatccagc tggagtcctt tctaagcagc cacagcctga |
| 38761 | tcctcccact tcctccccca agaaaacatt gtgggttgat ggYcataccc tgaggttctg |
| 38821 | gtccaaatcg gactttctat gaccttctgg gtctctagtg aaaactaaag actcctctcc |
| 38881 | agaaaaaaac atttggtttc taatgaggcc tggaatctta ttcttgacct ggggagcgga |
| 38941 | atcccttttt gcagtactcc cgggccctct gttggggcct ccccttcctc tccagggtgg |
| 39001 | agtcgaggag gcggggctgc gggcctcctt atctctagag ccggccctgg ctctctggcg |
| 39061 | cggggcccct tagtccgggc tttttgccat ggggtctctg ttccctctgt cgctgctgtt |
| 39121 | ttttttggcg gccgcctacc cgggagttgg gagcgcgctg ggacgccgga ctaagcgggc |
| 39181 | gcaaagcccc aagggtagcc ctctcgcgcc ctccgggacc tcagtgccct tctgggtgcg |
| 39241 | catgagcccg gagttcgtgg ctgtgcagcc ggggaagtca gtgcagctca attgcagcaa |
| 39301 | cagctgtccc cagccgcaga attccagcct ccgcaccccg ctgcggcaag gcaagacgct |
| 39361 | cagagggccg ggttgggtgt cttaccagct gctcgacgtg agggcctgga gctccctcgc |
| 39421 | gcactgcctc gtgacctgcg caggaaaaac acgctgggcc acctccagga tcaccgccta |
| 39481 | cagtgaggRa caggggctcg gtcccggctg gggtgagggg agggggctgg aagaggtggg |
| 39541 | ggaagggtag ttgacagtcg ctctataggg agcgcccgcg gacctcactc agaggctccc |
| 39601 | ccttgcctta gaaccgcccc acagcgtgat tttggagcct ccggtcttaa agggcaggaa |
| 39661 | atacactttg cgctgccacg tgacgcaggt gttcccggtg ggctacttgg tggtgaccct |
| 39721 | gaggcatgga agccgggtca tctattccga aagcctggag cgcttcaccg gcctggatct |
| 39781 | ggccaacgtg accttgacct acgagtttgc tgctggaccc cgcgacttct ggcagcccgt |
| 39841 | gatctgccac gcgcgcctca atctcgacgg cctggtggtc cgcaacagct cggcacccat |
| 39901 | tacactgatg ctcggtgagg cacccctgta accctgggga ctaggaggaa gggggcagag |
| 39961 | agagttatga ccccgagagg gcgcacagac caagcgtgag ctccacgSgg gtcgacagac |
| 40021 | ctccctgtgt tccgttccta attctcgcct tctgctccca gcttggagcc ccgcgcccac |
| 40081 | agctttggcc tccggttcca tcgctgccct tgtagggatc ctcctcactg tgggcgctgc |
| 40141 | gtacctatgc aagtgcctag ctatgaagtc ccaggcgtaa agggggatgt tctatgccgg |
| 40201 | ctgagcgaga aaaagaggaa tatgaaacaa tctggggaaa tggccataca tggtggctga |
| 40261 | cgcctgtaat cccagcactt tgggaggccg aggcaggaga atcgcttgag cccaggagtt |
| 40321 | cgagaccagc ctggacaaca tagtgagacc ccgtctatgc aaaaaataca caaattagcc |
| 40381 | tggtgtggtg gcccgcacct gtggtcccag ctacccggga ggctgagttg ggaggatcct |
| 40441 | ttgagccctg aaagtcgagg ttgcagtgag ccttgatcgt gccactgcac tccagcctgg |
| 40501 | gggacagagc acgaccctgt ctccaaaaat aaaataaaaa taaaaataaa tattggcggg |
| 40561 | ggaaccctct ggaatcaata aaggcttcct taaccagcct ctgtcctgtg acctaagggt |
| 40621 | ccgcattact gcccttcttc ggaggaactg gtttgttttt gttgttgttg ttgtttttgc |
| 40681 | gatcactttc tccaagttcc ttgtctccct Ragggcacct gaggtttcct cactcagggc |
| 40741 | ccacctgggg tcccgaagcc ccagactctg tgtaKcccca gcgggtgtca cagaaacctc |
| 40801 | tccttctgct ggccttatcg agtgggatca gcgcgggccg gggagagcca cgggcagggg |
| 40861 | cggggtgggg ttcatggtat ggctttcctg attggcgccg ccgccaccac gcggcagctc |
| 40921 | tgattggatg ttaagtttcc tatcccagcc Scaccttcag accctgtgct ttcctggagg |
| 40981 | ccaaacaact gtggagcgag aactcatctc caaaataact taccacgctg gagtgagacc |
| 41041 | acgaatggtg gggaggggag ggtcccacgg acatattgag ggacgtggat acgcagaaga |
| 41101 | ggtatccatg tggtggcagc cgggaagggg tgatcagatg gtccacaggg aatatcacaa |
| 41161 | actcgaattc tgacgatgtt ctggtagtca cccagccaga tgagcgcatg gagttggSgg |
| 41221 | tggggggtgt caaagcttgg ggcccggaag cggagtcaaa agcatcaccc tcggtccctt |
| 41281 | gttctcgcgt ggatgtcagg gccYccaccc accgagcaga aggcggactc aggggcgctc |
| 41341 | cagggtggct cgagctcaca cacgctgagt agacacgtgc ccgctgcacc ctgggtaaat |
| 41401 | acagacccgg agccgagcgg attctaattt agacgcccgc gaacgctgcg cgcacgcaca |
| 41461 | cgtgtcctcg gctcgctggc actttcgtcc cgccccctcc gtcgcgtgcS ggagctgacc |
| 41521 | cggaggggtg cttagaggta tggctccgcg gggtcaaaag gagaaggatc agtgagagag |
| 41581 | gcatccccac accctcccct agaactgtcc tttccccatc cagtgcctcc caaatctctc |
| 41641 | ttagtcccca aatgtatccc cgccctaagg ggcgctggtg ggaggagcta aatgtggggg |
| 41701 | cggRgctcgg agtccagctt attatcatgg catctcagcc agggctgggg taggggtttg |
| 41761 | ggaagggcaa cccagcatcc cccgatccca gagtcgcggc cggggatgac gcgagagagc |
| 41821 | gtggtcgccc ccagaaggcc ctgggccatc atgccggcct ccacgtagac cccaggggtc |
| 41881 | gctcactcct gccagctcgc cttcaccaag gccaggagct tagcgcacgc tcgcctcccg |
| 41941 | cccccccgcc gcctctgccg ccgccccctc cttggaaacc aagttaccaa cgttaaacca |
| 42001 | atccccaagc gcaactctgY ctcccccaca ccccacccgc cgcgccgcgc ggagccgtcc |
| 42061 | tctagcccag ctcctcggct cgcgctctcc tcgcctcctg tgctttcccc gccgcggcga |
| 42121 | tgccagggcc ttcgccaggg ctgcgccggg cgctactcgg cctctgggct gctctgggcc |
| 42181 | tggggctctt cggcctctca ggtaagagcY ccgctctggt tcggggtgga cagggcgggg |
| 42241 | gcggagtccc tggacctgag aaacggcctc ctgtccctcc cagctctgcc ctcgcctcgc |
| 42301 | tSccacgcct ctgcccccac ctcgagcctg agtcttctcc ccttccccct cctccccaac |
| 42361 | acacacccga gccccagctt cctgactcct cgatagcccc tacccgcttc gagatcctag |
| 42421 | gtgttcttcc gcacccaacc cttcgccctg gagacccagt gtctctcctg tccgctcccc |
| 42481 | gggtacctcc ttacgctStg ctgtgcacca tggtccacgR actggcatct tccccactcg |
| 42541 | cggtccgcga agactccatc tctccaacta ccctgactca gaggcgctgt tcccgctcca |
| 42601 | cccagaRccc tggcaaccgg gtccctcagc tgttcccgcg gttccttcca tgagcccagc |
| 42661 | cttgcgtccc ggcKccgtgt tcttcaccgg gtgtagggtc cttcctgatc cttgacccag |
| 42721 | cctcgtctct cctttgccct tgccgcaaac gcactctcct cgtatcccgg cattctgcca |
| 42781 | ggaccctgag aactggttca tcccccatcc cccatcccgg gtccccttct ctcagccttg |
| 42841 | ctgtgttcat ccaagaaccc acctttcctc tcctctacgc cctcccccca tgctttcccg |
| 42901 | ccgctccatc ggcgctttgg agaccatgSc tctctgctac cacgtcccag agacaccctc |
| 42961 | gaggtttaga ctctgggagt gcgcctttaa accggaggcc tgggcaSgac gcgggacgcc |
| 43021 | tgggttcttt ccctggctgS agcctctcct cctcctcccc gccctctgag aacccttgac |
| 43081 | tcgacatagg ggcgctaaga tgtcagggag ttggctcccc aggctcagcc cgcgtttccc |
| 43141 | tgggcagcgg tctcgcagga gcccttctgg gcggacctgc agcctcgcgt ggcgttcgtg |
| 43201 | gagcgcgggg gctcgctgtg gctgaattgc agcaccaact gccctYggcc ggagcgcggt |
| 43261 | ggcctggaga cctcgctgcg ccgaaacggg acccagaggg gtttgcgttg gttggcgcgg |
| 43321 | cagctggtgg acattcgcga gccggagact cagcccgtct gcttcttccg ctgcgcgcgg |
| 43381 | cgcacactac aggcgcgtgg gctcattcgc actttccgtg agttctgggt ggccacgcgc |
| 43441 | gtactccact actctccctc cctcccaggc cccgccccct gggtcccagg gtcctcccct |
| 43501 | tcaggcccca ccttctgttc caagtcccgg Ygttcaaaga gctgcggact cttccccctt |
| 43561 | gcagagcgac cagatcgcgt agagctgatg ccgctgcctc cctggcagcc ggtgggcgag |
| 43621 | aacttcaccc tgagctgtag ggtccccggc gccgggcccc gtgcgagcct cacgctgacc |
| 43681 | ctgctgcggg gcgcccagga gctgatccgc cgcagcttcg ccggtgaacc accccgagcg |
| 43741 | cggggcgcgg tgctcacagc cacggtactg gctcggaggg aggaccatgg agccaatttc |
| 43801 | tcgtgtcgcg ccgagctgga cctgcggccg cacggactgg gactgtttga aaacagctcg |
| 43861 | gcccccagag agctccgaac cttctgtgag tgggtgtggg gaggagatgg ggacccagtg |
| 43921 | gggtcggtcg gtgtttagga ggtttagagg tagatacatc tgaatgctga ccccgacttc |
| 43981 | aaccctcgcc ggctgagctg tttccccctc cgtgccttga ggRKgataat acgataagat |
| 44041 | agtgcatgtc aagtgcttag gacatattga gcgctcggag ttagttcaaa cttggttctt |
| 44101 | cgacccctag ccctgtctcc ggatgccccg cgcctcgctg ctccccggct cttggaagtt |
| 44161 | ggctcggaaa ggcccgtgag ctgcactctg gacggactgt ttccagcctc agaggccagg |
| 44221 | gtctacctcg cactggggga ccagaatctg agtcctgatg tcaccctcga aggggacgca |
| 44281 | ttcgtggcca ctgccacagc cacagctagc gcagagcagg agggtgccag gcagctgRtc |
| 44341 | tgcaacgtca ccctgggggg cgaaaaccgg gagacccggg agaacgtgac catctacagt |
| 44401 | aaggaaggag gcggggtctc cgcggctccg aggtgggacc agaggaatgc gaaggcgggg |
| 44461 | cgaagagtgg gcgggacctc agtaccggaa caggcgtgKc ccgaggggcg gggcaggtgg |
| 44521 | gggcggagac gtaatcgctg gggaggagga gcctgtacag cctgagaggc ggggcgccgt |
| 44581 | accctagttc gttctcagca ccccgaggat tcgggcagat aaggggcggg ccttgaccgg |
| 44641 | agggaggggt atggtcagta tactacgacc aaatgctccg cccccaggct tcccggcacc |
| 44701 | actcctgacc ctgagcgaac ccagcgtctc cgaggggcag atggtgacag taacctgcgc |
| 44761 | agctgggRcc caagctctgg tcacactgga gggagttcca gccgcggtcc cggggcagcc |
| 44821 | cgcccagctt cagctaaatg ccaccgagaa cgacgacaga cgcagcttct tctgcgacgc |
| 44881 | caccctcgat gtggacgggg agaccctgat caagaacagg agcgcagagc ttcgtgtcct |
| 44941 | atgtgagttg gtgataaccc ctcgcccccc accttctggt gacttccaag gacccgcctg |
| 45001 | ctYcctcacc gtgtcgtgga ggcggagcca tttcttacgt ctaagcctct gtaaccccac |
| 45061 | gccctgcccg cagacgctcc ccggctagac gattcggact gccccaggag ttggacgtgg |
| 45121 | cccgagggcc cagagcagac gctgcgctgc gaggcccgcg ggaacccaga accctcagtg |
| 45181 | cactgtgcgc gctccgacgg cggggccgtg ctggctctgg gcctgctggg tccagtcact |
| 45241 | cgggcgctct caggcactta ccgctgcaag gcggccaatg atcaaggcga ggcggtcaag |
| 45301 | gacgtaacgc taacggtgga gtgtgagtgg gggtgcgcag RgtgcaYttc tatctggttc |
| 45361 | aaggtctgga gggtggccag cctccaggga agagtaggag tagggtatga ggtgtccctt |
| 45421 | tgggtgaggt tttgggaaag ggaagaggct ggttagtggg gttggagaaa gatcttggag |
| 45481 | gatggaaggg accgggtggg cgtgccccta gcctagggcg tggtatttgg gcggagtcgt |
| 45541 | ggaaaggcgg gcagtccaga gtgtttaagt ttttagRcga aaaaggcgcc actggtggct |
| 45601 | caggaagctc ccagacagag tgcatgSctc gactagcgtg acacctcctt ggatcggcgt |
| 45661 | ccaagggtta tgcagggaca acacttcgtg gaagccttgc cgcgccaagg agggtctagg |
| 45721 | gacgtcagat ttgcccccaa accccaaagc caacaataca ctccctcctc cagacgcacc |
| 45781 | agcgctggac agcRtgggct gcccagaacg cattacttgg ctggagggaa cagaagcctc |
| 45841 | gctgagctgt gtggcgcacg gggtaccgcc gcctgatgtg atctgcgtgc gctctggaga |
| 45901 | actcggggcc gtcatcgagg ggctgttgcg tgtggcccgg gagcatgcgg gcacttaccg |
| 45961 | ctgcgaagcc accaaccctc ggggctctgc ggccaaaaat gtggccgtca cggtggaatg |
| 46021 | tgagtagggg caccgcggag ttaggcagga tctgtgggac aaccccggct ggacttcctg |
| 46081 | gcccccgtgt gagcccctgc aatcctgttt cccagatggc cccaggtttg aggagccgag |
| 46141 | ctgccccagc aattggacat gggtggaagg atctgggcgc ctgttttcct gtgaggtcga |
| 46201 | tgggaagcca cagccaagcg tgaagtgcgt gggctccggg ggcRccactg agggggtgct |
| 46261 | gctgccgctg gcacccccag accctagtcc cagagctccc agaatcccta gagtcctggc |
| 46321 | acccggtatc tacgtctgca acgccaccaa ccgccacggc tccgtggcca aaacagtcgt |
| 46381 | cgtgagcgcg gagtgtgagc gaggcccagg cgggtaggga gcaggggtgc cccacggtcc |
| 46441 | aggcactccc tgacatcccc catggctgYt ttgcagcgcc accggagatg gatgaatcta |
| 46501 | cctgcccaag tcaccagacg tggctggaag gggctgaggc ttccgcgctg gcctgcgccg |
| 46561 | cccggggtcg cccttcccca ggagtgcgct gctctcggga aggcatccca tggcctgagc |
| 46621 | agcagcgcgt gtcccgagag gacgcgggca cttaccactg tgtggccacc aatgcgcatg |
| 46681 | gcacggactc ccggaccgtc actgtgggcg tggaatgtga gtgggggcag caccggatgg |
| 46741 | aggggacacg gtcctcggaa gaatgactcg cagcggtggg agcattcaag ggcacctctc |
| 46801 | ccaatcccat tctcggggac agggaattcc agcctaaacc agggggtaat gaaaattcta |
| 46861 | gccaggcgca gtggctcagg tctgtaatcc caacactttg gaaggttgag gcggatgaat |
| 46921 | cacttgaggc caggagttcg agaccagccc ggccaacatg gcgaaaaccc gtctctacaa |
| 46981 | aaattagccg gtcgtggtSg tgggcgcctg tggtcccagc tacttgggag gctgaggcag |
| 47041 | gagaatcgct tgagcctggg aggcagaggt tgcagggagc cgagatcccg ccactgcact |
| 47101 | ccagcctggg caacagagtg agactctttc ttagaaaaac agaaaaagaa aattataggg |
| 47161 | aataggagca cggccctcct caaatcctgg attagaacac tgacctgggc ttcacctcct |
| 47221 | tccattcggt gaagaagggc gaggaatttt agccgcaaca gcagYctgat tgtcggggaa |
| 47281 | ggaggctctg ataggaggca ggatcctctt ctgcccatca gaggcgcggt ggtctcccat |
| 47341 | cgatcgttgt gggccggagc agggcatttg atcagtggct gggccggcgc taagccccac |
| 47401 | ttcaccttct gtgcccttca gaccggccag tggtggccga acttgctgcc tcgccccctg |
| 47461 | gaggcgtgcg cccaggagga aacttcacgt tgacctgccg cgcRgaggcc tggcctccag |
| 47521 | cccagatcag ctggcgcgcg cccccgRggg ccctcaacat cggcctgtcg agcaacaaca |
| 47581 | gcacactgag cgtggcaggc gccatgggaa gccacggcgg cgagtacgag tgcgcaScca |
| 47641 | ccaacgcgca cgggcgccac gcgcggcgca tcacggtgcg cgtggccggt MMgtggcagc |
| 47701 | tggggagagg cggggcgagg tatctgagag ggggcgtgac ctgggtcttg gggcggccgg |
| 47761 | ccccgcctgc ctccctctcg gtcccggKag actagRcggW agtgggacag aRtagaagtc |
| 47821 | aaaggtgcct tagcgggtgg ggctgttgat cgcactttga ggggtgggag atggaggtgg |
| 47881 | cagggggact gaattcaagg ggagggaccc tccggggctg tcactgctgc aggaaaggac |
| 47941 | tttaaaactt gggctggatt ctgcgtggYg gaaacttgac ccaattcaca atccactgtg |
| 48001 | gggaagattg gggagggacc aaagtcattg gatgWtggag ggagagaggg gtcaagatcg |
| 48061 | ccttctctca cttcctgaag ctcctggggc tgggttctct ctggggtcgg gggaacctga |
| 48121 | cctggtcctg ccggtcctca ttttttgggg gatggggaga gttgacttgt cgccaggggg |
| 48181 | ctaagtgtgg tcacgggttt atatccccat gggctgaagt gcgatataaa tcgggactcg |
| 48241 | ggtcgccctg gcactgggag tggccttatt gcatgggttg tctcccttct cccaaggcca |
| 48301 | gacagtgagc tccccggggc atgaggactg acttcgtgcc tgtgggagtg cggggggcgg |
| 48361 | tgggagaagc cccccggagc ttggccaccc tccgcgaggg tctccacccc gcaggtccgt |
| 48421 | ggctatgggt cgccgtgggc ggcgcggcgg ggggcgcggc gctgctggcc gcgggggccg |
| 48481 | gcctggcctt ctacgtgcag tccaccgcct gcaagaaggg cgagtacaac gtgcaggagg |
| 48541 | ccgagagctc aggcgaggcc gtgtgtctSa acggagcggg cggcggcgct ggcggggcgg |
| 48601 | caggcgcgga gggcggaccc gaggcggcgg ggggcgcggc cgagtcgccg gcggagggcg |
| 48661 | aggtcttcgc catacagctg acatcggcgt gagcccgctc ccctctcccc gcgggccggg |
| 48721 | ggacgccccc cagactcaca cgggggctta tttattgctt tatttattta cttattcatt |
| 48781 | tatttatgta ttcaactcca agggcgtcac ccccattttc taSccatccc ctcaataRag |
| 48841 | tttttataaa ggaactccct gtctccgctt ctgtttctgc aaggtggaac aagSccaggg |
| 48901 | ttccagtcgt gacctcccga gttattcatt caaWaagcga tttttgagag ggcctggggt |
| 48961 | ggtggcttag gcctgtaatc ccaggacttg tgggaggccc aggtgggagg atcccttgca |
| 49021 | cccaagagtt tcagaccagc ctgggcaaca tagggagacc ctacaaaaaa tatttttcaa |
| 49081 | aaattagcca aggccggtcg cggtggctca tgcgtgtaat cccaggactt tggaaggccg |
| 49141 | aggcggccag atcacgaggt cgaggtcatg agatcgagac catcctggcc aatatggtga |
| 49201 | aaccctgtct ctactaaaaa tacaaaaatt agctgggcat ggtggtgcRc gcctgtagtc |
| 49261 | ccagctattc gggaggctga ggcaggagaa tcgctttaac cccggaggca gaggttgcag |
| 49321 | tgagccgaga tcgtgccact gaactccagc ctggcgacag agcgagattc cgtcttaaaa |
| 49381 | aaaaaaccag gcgtggtggc tcacgcctgt aatcccagca ctttgggagg ccgaggcggg |
| 49441 | cggatcacga ggtcgggaga tcgagaccat cctcgctaac acggtgaaac cccgtctcta |
| 49501 | ctaaaaatMc aaaaaaaaaa aaaaaaaaaa aattagccga gcgtggtggc gggcgcctgt |
| 49561 | agtcccagct actcaggagg ctgaggcagg agaatggcat gaacccgggg agcagagctt |
| 49621 | gcagtgagcc gagatcgcgc cactgcactc caccctggca gacagcaaga ctccgtctca |
| 49681 | aaaaaaaaaa aaaaaattag ccagggccga gcacagtRct ctcgcttgaa tcccagaact |
| 49741 | ttgggaaact aaggcggggg gggggggtca cttgaggtca ggagttcgag accaccctgg |
| 49801 | ccaacgtggt gaaacacctt ctctaccaaa aatacaaaaa aattaaggcc gggtgaagtg |
| 49861 | gctcacgtct gtaatcccag cactttgcga ggctgaggcg ggcggatcat ggggttagaa |
| 49921 | gatcaagact gtcctggcta acatggtgaa accccgtcac taMtaaaaac acaaaaatta |
| 49981 | gccgggcgtg gtggcacatg cctgtagtcc cagctactca agaggctgag gcaggagaat |
| 50041 | cgctttaacc ccggaggcag aggttgcagt gagccaagat cgcaccactg cactccagcc |
| 50101 | tgggcaatag agcgagactc tgtctcaaaa aaaaaccaaa aacaaaaaca aaaaaataca |
| 50161 | aaaatttagc tgggctgtag tggcacacgc cctgtaatcc cagctactcg tgaggctgag |
| 50221 | gcaggagaat tgctagaacc cgggaggtag agatcttgcc attgcactcc agcctgggca |
| 50281 | acagaactag actttgtctc aaaaaaaaaa aaaaaaaaaa aaaagtgttt tttgaggggg |
| 50341 | ctgggcatgg tggctcaagc ctgtaatccc aggacttttg ggaggcccag gtgggaggat |
| 50401 | cccttgtgcc cacgagtttc agaccagcct gggcaacata gcgagaccct acaaaaaaac |
| 50461 | atgtttcaaa aaaaaatttt tttttttgag atggagtctc gctctgtcgc ccaggctgga |
| 50521 | cagcagtggc actatctctg ctcactgcaa gctccgtctc ccgggttcat gccattctcc |
| 50581 | tgcctcagcc tcccaagtag ctgggattac aggcgcctgc caccacaccc agctaatttt |
| 50641 | tttttgtatt tttagtagag atgggatttc accgtgttcg ccaggatggt ctctattcct |
| 50701 | gacctcgaga tccacccgcc tcagcctccc aaagtgctgg gattacaggc gtgagccacc |
| 50761 | gcgcccggcc tcaaaaaaaa aatttttttt ttgagatgga gtcttgctct gtcacccagg |
| 50821 | ctggagtgct gtggtgtgat cttggctcac tgcaacctct gcctcctggg ttcaagcaat |
| 50881 | tcccctgcct tggcttccca aatagctggg actataggca tgcaccacca cacccagcta |
| 50941 | atttttgtat ttttagtaga gacagggttt caccatgttg gccaggctgg tctcgaactc |
| 51001 | ctgacctaag gtgatccacc tgcctcggtc tcccaaagtg ctgggattac agctgtaagc |
| 51061 | caccatgcct ggtgtatttt tcaaaaatta gccagacatg gtggtgcatg cctgtagtcc |
| 51121 | cagctacttg ggaggctgag gcaggaggat cctttgagcc taggaggtca aagccacttc |
| 51181 | cagctatgat atYgtcattg tgctccagcc caggtgacag aaccagaccc tgtctctaaa |
| 51241 | aacaaacaac gaaaaaatcc aaaaactttt tttgaacacc tactatgcat caggcacaat |
| 51301 | ctaagctgtc tttttctttt tctttttttt ttttttttga gacggagtct ctctgtcgcc |
| 51361 | caggctggag tgcagtggtg taatctcagc tcactgcaag ctctgcctcc cacgttcaag |
| 51421 | cgattttcct gcctcagcct cctgagtagc tgggattaca ggcgcgcgcc accactacgc |
| 51481 | ccggctaatt tttgtatttt tagtagagac agggtttcac catgttggtc aggctagtct |
| 51541 | ggaactactg acctcgtgat tcacctgcct cggtctccca acgtgctgga attacaagcc |
| 51601 | tgagccactg tgcctggccc ggctggctgt cttttctcag tgtgacctag agctcgtcct |
| 51661 | ctctcagtgc ctgttttctg ctctcttcac acccttgggc aggggtgggg gcctcactcc |
| 51721 | ttcctcctac ttatctcttc cacccagcac tgccctctga tccgggtcac tcttggaggt |
| 51781 | gggggctagg tgcttccccc aggcctggtt accggcagag ctgaggccgc cttgccaggg |
| 51841 | tgggtggagg ccctctccgt tgtgcccctg ccctgagact tcggtgagac cttcctgggc |
| 51901 | aggcactggc tgatgctatg ggtgtcagcc cttagcatgt cctccctgtt aaagggggca |
| 51961 | cccctgcccc gccctcaatg cctcctttgt cctgtgtcca cccacagtgc ttcagtttac |
| 52021 | ccccaccctg cctttccact cagcccatca tgattcagaa cagacacaca cagggtatcc |
| 52081 | gctgcaaccc ccactacacg gcaccttccc ccctcatccc caacagcata aggcacagca |
| 52141 | gctgggcctc ctccccacag cctcctcctc tgcccctcct ccctcaaaag caggaacacg |
| 52201 | gagccctaga gaaggaagcc agtcccatgc aaacatgtaa tgagcaaaga tgagacgggg |
| 52261 | aaatggcaca agagggcagg ccagtgctcg gtgctggagc caggggccag gtataacacc |
| 52321 | caaaaaggca ccagccaggt gcagtccctg ccactaacag cacctcacct tttctgcctg |
| 52381 | tgccctccct acttaatgat ctctctccgc tcctactgac ctcaaggtct ctttcagagc |
| 52441 | cttcagatga gatatttatt ttagtaggtt ggtgcaaaag taattgctgt ttttgccatt |
| 52501 | gaaagtaatg gcaggcaggg catggtggct cacgcctgta atcccagcac tttgggaggc |
| 52561 | cgaggtgggc agatcacgag gtcaggagtt caagaccagc ctggctaaca tggtgaaacc |
| 52621 | ctgtctctac taacaataca aaaaaaatta gccggacatg gtggcacacc cctgtagtct |
| 52681 | cagctacttg ggaggctgag gcaggagaac tgcttgaacc ctgaagcgga ggctgcagtg |
| 52741 | agctgaaatc acgccactgc actccagcct gggccacaga gtgagactgt ctcagaggaa |
| 52801 | aaaaaaaaaa aaaaagtaat ggcaaaaact gcaattactt tttctttttc tttctgtttt |
| 52861 | tttttttgtt tgtttgtttg tttgtttgtt tgtttgagac ggagtctcac tgtgtggccc |
| 52921 | aggctggagt gcagtggcgc aatcttggct tactgcagcc tccgcctccc aggttcaagt |
| 52981 | gattctctcc tgcctcagcc tcccaatagg attacaggca cccaccacta tgcctggcta |
| 53041 | atttttgtat ctttagcaga gatggggttt caccatgttt gccaggctgg tctcgaactc |
| 53101 | ctgatccacc cacctcagct tcccaaagtg ctgggattac aggcaggcat gagccatggt |
| 53161 | gcccgacatt tttttttttt tttctttttt tttttgagac agagtctcac tttgtcaccc |
| 53221 | aggctggagt gcaaaggcaa gatctcactg cagcctctgc ctcctgggtt aaagcgattc |
| 53281 | tcccacctta gccccctgag taggtgagat tacaggcaca tgccaccaca cctgcctaat |
| 53341 | ttttgtattt ttaatagaga gggggtttca ccattttgtc cacgctggtc tcgaactcct |
| 53401 | gacctcaagg gattcgccca cctcagcctc ccaaagtgct gggattacag gcatgagcca |
| 53461 | ccacgcccga cctccttaac tcttttattt ccttgcacac tttttctctg tagcccagct |
| 53521 | cacaacttaa catcctctta gacatacacc tgaggttttt tttttttttt tttttgagag |
| 53581 | ggagtctctc tctgtcaccc aggatggaat gcagtggtgt gctctcagct cactgcaacc |
| 53641 | ttgcaaccac tgcctcccag gttcaagcaa ttctcctgcc tcagcctccc gagtagctgg |
| 53701 | gactacagac acatgccgcc acacctggct gattttttgt tttttagtag agacagggtt |
| 53761 | tcaccgtgtt gcccaggctg gtctcaaact cctgagttca ggcagtccgc ctgccttagc |
| 53821 | cttccaaaat gctgggatta caggcatgag gcaccgcgtc ctgcctctga gttttttttt |
| 53881 | tttttttttt tttactgtct cctagaacat cagccccagt agagcaggga tctttgttct |
| 53941 | gttcaccact gagggtcctc attgggtcct agcacacaca gttgctcaat aaatgttgaa |
| 54001 | taagtgggta aagacagcca cgagcttgca gatatgtgtt caaggtgtgt ccttgcagag |
| 54061 | agcttctcta tacttggcac tggagaggcc tgtcgtgggc aaggacatag atgtggcgac |
| 54121 | ctcagacttg agaactcctg gggcagtagg ggagatggat gtggataatg taaccataag |
| 54181 | ccttcctttc tattttagac tgagtggtca aggaaggctt ccagacaatg gaaattctga |
| 54241 | taggttctaa gagggagacc agcaaggagc tgaaccggag gctgacacag ggtgagagtg |
| 54301 | ggaatgtcta ttttattttt tttttggaga cagggtctca ctctgttgct caggcagcag |
| 54361 | tgcagtggca cgatcgtggc tcactgcagc ttcaacttcc cagactcaag tgatcctccc |
| 54421 | acctcagcct cccaagcagc tgggaatata ggtgcatgcc accacatccg gctaattttt |
| 54481 | gtatttttgg tagaaacggg gtttcaccat gttgcccagg ctggtcttga actcctgggt |
| 54541 | tcaagtgatc ttgctgcctt gggctcccaa agtgcttgga ttacaggtgt gagcctgtga |
| 54601 | gccacatctg gcctatttta ttttttaatt agttttttgt ttttgttttt gttttgagat |
| 54661 | ggagtctctg tctccaaggc tggagtgcag tggcacgatc tcggctcact gtaacctcca |
| 54721 | cctcccaagt agctgggatt acaggcacat gccaccacgc ctggctaatt tttgtatctt |
| 54781 | tagtagagac agggtttccc catgttggcc aggctggtct cgaactcctg acctccggtg |
| 54841 | atccacctgc ctcagtttcc caaagtgctg ggattatagg aatgagccac tctgcctggc |
| 54901 | tgggcatgtg tcttgttttt ttcattctgt agataacaag caattgtcct ccctcagcct |
| 54961 | cccaagtagc tggtactaca ggcgcctgcc actacgcccg gctatttttt gtagttttag |
| 55021 | cagaggtggg gtttcaccat gttaaccagt ctctaactcc tgacctcagg tgatcctcct |
| 55081 | gccttggcct cccaaagtgc tggaattaca ggtgtgagcc accaagccca gcctcaaact |
| 55141 | cctgagttta agtgatcctc acacctcagc ctccctgagt gctgggatta caggtgtgag |
| 55201 | ccaccacacc cggcctgagt cgggggaggt gtctatttta gtctgagtgg tcaaggaagg |
| 55261 | cttctctgaa gagctgatgt tggggccagg cctcctcaag cgattagaag gtgtcagcca |
| 55321 | tggggagatg tggagggagg gtgtttcagg ccaaggaagg gtaagatcaa ataattcagg |
| 55381 | gatctgaggg cagaggaatc tggactccag ttctcccatt cagggctgcc caggagagac |
| 55441 | aaagaggatc tcataagggt gtgtggcttt gtggctcact cacgcccaca gcaaggacca |
| 55501 | tgcccaccct actttttttt ttttccccaa gtctccctct attacccagg ctggagtaca |
| 55561 | gtggtacgat cacggttcac tgcagtcttg acctccaggg ctcaagtgat cctcccacct |
| 55621 | cagtgtcctg agtagctggc actacaggca tgcaccacca cccctggctc attttttgat |
| 55681 | tttttggtag agacggggtc tcactgtgtt acccaggctg gtctcaaact cctgggctca |
| 55741 | agcgatcctc ccacctctgc cttctgaagt gttgggatta caggtgtgag ccaccatgcc |
| 55801 | cagcccgcca ccagtcttga ctctccatcc tccctccctc cctgtgagtg ctggtggcct |
| 55861 | gtgtgtcccg cctgctgaaa gatgcctctg acttggggct tttgacttca ggcccgggag |
| 55921 | acacattttg gctggattag ggagatctta aaagggggta ggggacacag caggggctaa |
| 55981 | ggagagtgac acactgcagg caggggttgg gtgacagatg aaggatccac tggtatatgc |
| 56041 | tgccagtgtg tgccatctac ttgtgtgttt tacgtgtgtc tggtgcatgt gtcctgtgtg |
| 56101 | ggctgtatct gtgtgtgact cagctacgtg tcagcatatc cctgtcgacg tgtgtctctg |
| 56161 | ctgatgtgtc tgtctgcacg cacggctgtc ttgcatgtgt cttttttgag atagagtctc |
| 56221 | gctctgtcac ccaggctgga gtgcagtggc acgatcttgg ctcattgcaa cctcaacctc |
| 56281 | ccaggttcaa gcgattctcc tgcctgagcc tcccaagtag ctcggattac aggcacatgc |
| 56341 | taccacgcct ggctaatttt tgcatattta gtagagacgg ggtttcatca tgttggccag |
| 56401 | gctggtctcg atctcctgac ctcaggtgat ccacctgcat cagcctccca aagtgctggg |
| 56461 | attacagaaa tgagccactg tgcctggcta gacatgtgtc tttttttttc ccttctgtat |
| 56521 | ataacagggt ctcactatgt tgcccaggct ggtctcaaac tcgtggcctc aagcaatcct |
| 56581 | cctgcctcag cctcccaaag tgttgggatt acaggcgtga gccactgcac ccggcttgtg |
| 56641 | tgtgtctttc tgtatctgtt gtgtgtgtct gtgggtagtg gctgtttccc tgcatgggac |
| 56701 | attgtggtaa atgtggctaa ttccctgtgt gggggctgct gcctgtggat aagactgtgt |
| 56761 | ctctgtgcat gcgcacgtgt gtgcacgccc ctcacaaccc caacgagaaa acacctgtcc |
| 56821 | accttcctgg gctggcacag gggcacagga ggcgggatcc caaatcacag gctttttctc |
| 56881 | tcggcatatc tctgtacttt attgtccctg ctgtaacaat gctcatcctt cctgagagcg |
| 56941 | tctcctgagg gggcctcggc caaggctgac tggagaaggg gctggtgacc cctagcaggc |
| 57001 | tctgccacct gaggcctggg tcttcccccg ggatccttga atctggagat ggcagagagg |
| 57061 | aggcaggccg gctctttccc caatcctcct aggagagctg cttctgccca ttctcccact |
| 57121 | ggtgaaacgg aggcagaagc agcagctcag cagggtgaag ctgggtttaa ccttcctgaa |
| 57181 | Yggggtccag ggacactcca catctgccac atagcctctt gggctggaca tttttcctgg |
| 57241 | gcaccagcca gcagctggag cagcgagtgc cgtatttctt gtacctaggg ttgggggaca |
| 57301 | agaaactgcc atttaggatg cagtgggggc ccggaaaccg ccacaaggaa accacttttc |
| 57361 | ccaagaggta ggtgttttgc tttttgcttt ccccacaggc catcctggtt acacgtggac |
| 57421 | tgatttgggg acccccgccc caactccctc ctccattcta aggacctgat cccacaggcg |
| 57481 | ttgcagagag gggtcccatc ttcagcgtct ctccagagcg gggtcctctg ggtccgacag |
| 57541 | gaagcacagc gccggggctc tgaagggtca gaggtcaaag ggcaggggtc agaggccaag |
| 57601 | catgtgagct cgggatgcct gtgcggagtc gggcattttg tgggggtctc agtaaaggcc |
| 57661 | ccacctagct ctggatcagc cctgggagtt gccagctcta agccacagca aagccaggaa |
| 57721 | gggagacaga tggcagcatt ccgcagagga ggaagctggg ggtgggggtg actcagccca |
| 57781 | agtggagggg ggtgctgcga ctcctccctg agggctctaa atggggaagc aagatggaga |
| 57841 | aggggggggc agggagaaag gcagggaaga caggaaattg gcccccaaaa tatttatagc |
| 57901 | tcttgggttt tcaggactca cccagggcct cgctgcctgc tgagtgggcc tcggtgcctc |
| 57961 | ctgggtgggc tgcagggccc ccaacagcat ctgcagggga ttcctgagaa cggctactgc |
| 58021 | agggcaggct gtggggcaga caaggtatta gcactggggg ggatctgtag cttgtcccca |
| 58081 | acagccctcc gaaatgaaga tttagtatca aggtatcagc catcccccga ggaggcaact |
| 58141 | aatatctgaa tggcctggct ttgcctctca ttagtaatat tattattatt attattatta |
| 58201 | tttttgcaga tggagtctcg ctctgttgcc caggctggag tgtagtggtg tcatctcagc |
| 58261 | tcactacaac ctccacctcc cgggttcaag caattcttga gcctcagcct cccaagcagc |
| 58321 | tgggactaca ggcgcgcgac accacgccca gctaattttt gtatttttag tacagacggg |
| 58381 | gtttcaccat gttggccagg ctggtctcga actcctgacc tcaagtgatc cacccacctc |
| 58441 | ggcctcccaa agtgcaggga ttacaggcat gagctaccgt gcccgaccta attattatta |
| 58501 | ttattatttg aaaaatagta agcacaggga cagcctgcca ggttccaatc ccagttctcc |
| 58561 | atgtccttgc tgtgtgagcc tggacacatt atctcctact ctgtgcctca gtttcctcat |
| 58621 | ctgtaaaatg ggcttcccaa tacaacagtt tttttttctt tgaaaatttg atttattgat |
| 58681 | ttttaaaaag agatgggagt ctgggcaata tagggagacc ccgtctctac aaacaaaaaa |
| 58741 | aatagccagg agtggtggta caggcctgca ctcccagcta gttgggaggc tgaggaggga |
| 58801 | ggatggcttg ggcctgggag gtccaggctg cagtgagcca tgattgcgcc actgcactcc |
| 58861 | accctgggtg acagagcaag accctgtctc aaaaacaaac aaacaaacaa aagcaaaaaa |
| 58921 | aagcagccag gcatggtggc ggctcactcc tgtaatccca acactttggg agggcaaggc |
| 58981 | agatggatca cctgaggtca agagatcgag accatcccgg ccaacatggc aaaaccccat |
| 59041 | ctctactaaa aatacaaaaa ttagccaggc gtggtagcag gcacctgtaa tcccagctac |
| 59101 | tccagaggct gaggcaggag aatcgtttga atccgggagg cagaggttgc agtgagccga |
| 59161 | gattgtgtca ttgcactcca gcctgaacga cagagtgcaa ctccatctca aaaaaaaaaa |
| 59221 | aaaatattgt tttggcggca tgattaaaaa agtcataatt ataattttta taataacaat |
| 59281 | aaataggtaa aaattagagc tgggggtcat actatgttgc ccaggctggt cttgaactcc |
| 59341 | tgggctcatg caatcctccc accttggcct cccaaagtgc tgggattaca ggtgtgagcc |
| 59401 | accaggccca gcttcccaaa gctttttttt tttttttttt aaggggatgg agtcttgctc |
| 59461 | tgtcacccag gctggagtgc agtggtgcca tctctgctca gtgcaacctc cacctcctgg |
| 59521 | gttcaagcga ttctcctgcc tcagcctccc aagtagctgg gattacaggt gcccattacc |
| 59581 | attcccagct agtttttttt ttttttttga gactgagtcc cactctgtgg ctcaggctgg |
| 59641 | agtgaggtgg cgctatctct gcttactgaa gacttcacct cctgagttca agcgattctc |
| 59701 | ctgcctcagc ctcctgagca gctgggatta caggcaccca ctacaacgcc cagctaattt |
| 59761 | tttgtatttt tagtagacat ggggtttcac tatgttggcc aggctgatct caaactcctg |
| 59821 | acttcaggtg atctgctcgc cttggcctcc caaagtgatg ggattatagg cgtgagccac |
| 59881 | tgtgcccggc ctccccaggc ttttgaatga gcccatacac ataaaatact gcctgttaac |
| 59941 | ctcttattgt tgtcactacc aacaaatagt agctgttagc attattggga aaaatcaccg |
| 60001 | cagccgcagc tgcagcctaa gcaaggccag gcctgattcc taaagagacc aggtctcaca |
| 60061 | ttgggagtgg ggagccactg agtcaggagc ggcaggactg tatctggcac cagctcaagt |
| 60121 | tgtcaaccag agactgggcc acgtgtaacg tagagcacag gtgagggtcc acaccgacgc |
| 60181 | cagRgagcac ccctacctcc cacactggtg tttgcgttgt aactgggaga aagggcggcc |
| 60241 | tggccccagc cggcccctac ctgtacgtgg ggatgatctg caggctggag tccggcttta |
| 60301 | tctgaaactt cagagtcacc ccctcgaacc cagggtccac tctctcggtg ccccggcagg |
| 60361 | ggttcagctg tttccggggc cttctctggg gtggggccga gggagtcccc gggggctgca |
| 60421 | gacggcggcc cttttggctg atcctggtct gtgtgtcctt ggaatccctg tccagcagca |
| 60481 | ggcggccctg agtccccaga gccatcgggt cccagcaagg ccccaggacY ggggtgtcct |
| 60541 | gggcagggga ctgacccagc ctctccactg tctcttggag gaagcacagg gcggtgaccg |
| 60601 | actcctggca tgcaggccag agggacctgg ggagaagggc agtggggtca cgttttcttg |
| 60661 | ggtgactcct gggtttagcc ggccaggggg ttccaacttg ggccggctct ttgtgtaccc |
| 60721 | ttggacaggc tacacacccc acctgaggtt cagtcatttc taactctggg tggaaggttt |
| 60781 | aagagttgca gtaattgcaa ctcacctcgc ccactgtggg cctcccaggc acgcccagcc |
| 60841 | ctccacatca atcaacccct tacggcaccc gtagatcacc caggcaggga ccgccctagg |
| 60901 | tgaccgaggc aggatatcaa gcctctggct gcaggatcga gaaaaaggtt actttttttc |
| 60961 | tttgtgtttc ttttagagac cgagcctcac tctgtacccc cgcctggagt gcaggggcgt |
| 61021 | gatcaggact cagtgcagcc tagaactcct gggctcgacc gatccttccg cctcagcctc |
| 61081 | ccgagtagct gggacttaca ggcacgcgcc accacgccgc tggcttgcca taatatgatt |
| 61141 | acgattttta tcattttcat tattattgag ttgccctcca taacccctgg atgatccctg |
| 61201 | gggagtgcct acatcagccc cagagacatg gaattgcatg gcctctgatc cctagatgac |
| 61261 | cccaagtgtc tctgggcacc agaacctctt tgtaagtttc agtaactatt ataaaattgc |
| 61321 | cacttctcga taaccccagc aaagagaggt catRacccct tgatcactga gccaggtggt |
| 61381 | gaagtgctag cggggtccgg ggaatggggt ttggccttac gcacctctca ctgattctgc |
| 61441 | agcatcctgg ggtcctcggc ttctcctggg agggggatcc tcccagccgc tccaggttca |
| 61501 | gacagggcgg cgccagaagc tttgctggca ttgaggagtc cgggattggc tcggcctcca |
| 61561 | ttgcgcccca gcccagcccc ctccctaatt tcacccggcc ctgtcccgct gggcccaccc |
| 61621 | gatgaaaccc acctgggagg ggcgggacct gtgtggtggg agcgctcttg gtagaggatg |
| 61681 | cgatgagccc cagctgagag cgaccagcct cggcggcacc tttcctcttt ctggcttttt |
| 61741 | tcaggtcatc cgcgcacccc tgcgtggcaa gggcggcctg gaccccacca aatccctccg |
| 61801 | cagctgccgg ggcggggcct tccacgctag ccctccccct cggaaatggg ggctcccagg |
| 61861 | cctctgcctt ggatccccag cggcatctta gatcccagag ggaaaagaga accgggggcc |
| 61921 | cagcacagca gatgcaggtg tggtttgttt ttgtttttgt tttttttttt tttgagacag |
| 61981 | ggcctcactc tgtcacccag gctggagtgg aatggcctga tcaccgctcc actgcagcct |
| 62041 | aaatcccctg ggctcaagtg atcctcccac ctcagtctcc cgagtagctg ggactacagg |
| 62101 | cacttaccac cactccacgc taatttttac tgtttttata gagacgaggt ctcgccatgt |
| 62161 | tgcccaggat ggtcttaagc tcctggcctc aagtgatcct cccatcttgg cttcccaaag |
| 62221 | tgctaggatt acaggtgtga gccacctgta gaccccagat ctgtccccca aggacactca |
| 62281 | acatgctggg gcctgtgtta tcgatttatt gcagctccaa tatgagtcca ctcctactca |
| 62341 | aaccctgatt ctcaggggcc tggggaaggc caccccacta ggggccctgt ccccaccaga |
| 62401 | gcctggacat gggactctct cccaagcagg ggtgttgtcc ttcacagggg cttccacgtc |
| 62461 | tctcccgggc ctgtccgcac tctgggcagg gctggcacct ggctattccc tcaatctggg |
| 62521 | ccctggggcc atggcaatgt ggaatggtcc aggtgttcat gtcagtgggg cttggggaca |
| 62581 | tggccatcca cgtagaagtt cctggtgatc ttgggMaggg tgaattccgc tccttccagc |
| 62641 | tccggtgtca gcacaatctg gcagcccagc cgcgagttct cctggaggag gggggccatg |
| 62701 | tctagcatgt cgtcttccct agggtggtga cacgggcagt gttagccgag ggcaagctag |
| 62761 | ctgggtgggt ggggggccag aggtSgggga gggaggaagc tgactgagcg cagggggtag |
| 62821 | tggtggggga ggcagctccc attccaggaa ctggaagtgg ggaggatggt gggggctgct |
| 62881 | gaatgctcca gggtgaagct gccagctaga cagggctgac ccactcacct ctcctcggga |
| 62941 | ggaggcagga gatccaggtg gtcttcactc acatacacat ggcaggtgga gcaggccagg |
| 63001 | gaggcttcac aggcccctag gggtgggaag tgaagggggc gcaggtgagt ggcagagtca |
| 63061 | ggaaggggct gctatgcgaa tggcgtagag gagtagacct ctcttccacc acgtgcctca |
| 63121 | cgtctccctc ctgggcctgc agctccactg tgtgtaccag gcactgtgtc atgtcctcaa |
| 63181 | tgtcctcagc tagctgttcc ccagtgtgta gggtttacaa acggacatct gggaggcccc |
| 63241 | atttcccagg gactccctag caggctgaga tgcagaacca ttaaggagca gggacttggc |
| 63301 | tgccaggtag ctgagagggg aaaggccagg caggctcccc gcagtctgtg agcccttgct |
| 63361 | gtgaccctat cactgtgctg ggctaatgtg tccccacctc tccactgctc ttggaggatg |
| 63421 | gaccggctga ctcagcagtc agtgtggtgg gggaaggaat caggcattag gattttgttt |
| 63481 | tttttttttt tgagatggag tcttgttctg ttgcccaggc tggagtgcag tggcacgatc |
| 63541 | tcggctcact gcaagctctg cctcccgggt tcacgccatt ctcctgcctc agcctcccaa |
| 63601 | gtagctggga ctacaggtgc ctgccaccac gcctggctaa tttttttata tttttttagt |
| 63661 | agagctgggg tttcaccatg ttagccagga tggtcttgat ctcctgacct catgatctgc |
| 63721 | ccgcctcagc ttcccaaagt gctgggatta caggcgtaag ccactgcacc cggccaggat |
| 63781 | tttttttttt tttttttgag acagggtctc actctgttgc ccagcctgga gtgccatggc |
| 63841 | tcagtcatag cttactgcct caacctcccg gctcaagcga tcctcccacc ccagcctccc |
| 63901 | gagtagctgg gactacatgt gcgcaccacc acacctggct aatttttttt tttttttttt |
| 63961 | tttgaggtgg agtttcgctc tctttgccca ggctggagtg caatggtatg atctcggctc |
| 64021 | aatgcaacct ctgcctccca ggttcaagtg attctcctgc ctcagcctcc caagtagctg |
| 64081 | ggattacagg tgcccaccat cacgcccagc taattttgta tttttagtag agatggggtt |
| 64141 | ttgccatgct ggccaggctg gtcttgaact cctgacctca ggtgatccac ctatctcggc |
| 64201 | ctccctaagt gctgggatta caggtgtgag ccaccgcacc tggccatatt tttttttttt |
| 64261 | tttttttttt ttgtagagac gaggttcacc atgtggccca ggctggtctc aaactcctaa |
| 64321 | gctcaagcaa tctgcctgcc ttggcttccc aaaatgctgg gactacaggc atatgccact |
| 64381 | gcaccagacc aggaattagg gttgaaagag acagaacctg aagttttctg ctgaccacct |
| 64441 | gcccttggtc acacccccac atcctctgat cacagagacc ggtgcttggg gatcatgtcc |
| 64501 | ctcctggcct aaaactttcc aaagggtttt cacactcaga ataaaatcca aactccttca |
| 64561 | cttagtcttg cagttccccc atccccctca ttcattcctt tgttttcctt tgtaattttt |
| 64621 | ttcttagaga tggagtcccg ctctgttgcc caggctggac tgcagtggcg tgatctcggc |
| 64681 | tcactacaag ctccgcttcc cggattcacg ccattctcct gcctccgcct cccagagcag |
| 64741 | ctgggactac aggcgcctgc caccatgcct ggctaatttt gtttttgtat ttttagtaga |
| 64801 | gacagggttt caccatgtta gccaggatgg tcttgatctc ctgacctcgt gatccgctcg |
| 64861 | cctcagcctc ccaaagtgct gggattacag gcgtgagcca ccacaccttg ccaggccact |
| 64921 | tacctctact agcccggcac agtggctcat gtctgtaatc ccagcacttt gggaggccaa |
| 64981 | ggcaggcaga tcaactgtgg tcaggagttt gagaccaccc tggccaacat ggcgaaaccc |
| 65041 | catctctatt aaaaatacaa aaacttagcc gggcatggtg gcagttgcct gtaatcccag |
| 65101 | ctactcggga ggctaaggca ggagaatcac ttgaaccctg gaggtagagg ttgcagtaag |
| 65161 | ccaagatcat gccattgcag tccagcctgg gtgatagagc gagactctgt ctcagataaa |
| 65221 | taaatgaaca aataaatcta tctcacattc caatccctgt cctagactat tggctgctgg |
| 65281 | attccagaac agaactttag gtaggccttt cataacccct cccaacatat cttccccttt |
| 65341 | aatgataaaa tcaggatccc tgatatattc atcccaaaag caaaccctat atattttctt |
| 65401 | ccagacaacc cttcccaagt tggtcataat tatgtattta cctgtgggat tattgtccct |
| 65461 | attagaccga aatggatgtg agatgacaga ggccttattt gttttgtata ttgctgcctg |
| 65521 | acgcgcagta ggtattcata gaaaggcagc acagattagt ggctacaatg gactggtgag |
| 65581 | tctagtattg tcactcactt gcccttagac caggtctact taacctgggg tttagttgcc |
| 65641 | tcatctataa tagggaataa cagtctctac tactcaagtt gtcatgaaga ttcagtcagt |
| 65701 | gaatatttat taacatggtt aaagacatat atatatatat atatatattt tttttttttt |
| 65761 | tttttttttt tttttttttt gagacagtct cactctgtcg cccaggctgg agtgcagtgg |
| 65821 | cgtgatcttg gctcactgca agctccacct cccgggttca tgccattctc ctgcctcagc |
| 65881 | ctcccgagta gctgggacta caggcgccca ccaccacgcc cacctaattt tttgtatttt |
| 65941 | tattagagac ggggtttcac cattttagcc aggatggtct ctatctcctg acctcgtgat |
| 66001 | ccgcccgcct cggcctccca aagtgctggg attacaggcg tgagccaccg cgcctggcct |
| 66061 | tttttttttg tttgtttgag acagagtccc gctctgtcat gcagcctgga gtgcagtggt |
| 66121 | gccatcttgg ctcactgcaa cctctgcctc ccaagttcaa gtgattcttg tgcctcagcc |
| 66181 | tcccaagtag ctgggattat atgcatgtgc caccacgccc agctaatttt tgtgtttctt |
| 66241 | gtagagacag ggtttcgcca tgttggccag actagtctca aactcctgtc ctcaagtgag |
| 66301 | tcgcctgcct cggcctccca aagtgttggg attacaggca tgagccactg Ygcctggaca |
| 66361 | agaaatttct attattattt ttctctcaac aaattatcaa acatctgtta catcttgaca |
| 66421 | ggcaacagat aagcatgaat aaggacatgg gtcaccaatc tcactggggg acagatgaca |
| 66481 | aagtaggaaa caaaacagcc tggcattgtg gcttgtgcct ataatcccag ctacttggga |
| 66541 | ggctgaggtg ggaggatcac ttgaacccag gagttcaagg ctgcagtgag gtatgattgc |
| 66601 | atcatggcac tccagcattg gcgacacagc aaggctccgt ctcttgaaaa aaaaaaagtg |
| 66661 | agaagtgcta tatatcatat cacactgata agacagagta actagggcat gggtacttca |
| 66721 | gctgggctga ggggaatggg attgttaggg acatgacatt tacactgagg tctgaaggat |
| 66781 | gagaaagagc cagaatgttt gctgggaaag cacttcagac agagggaaca gcaaatgtaa |
| 66841 | aggccccgaa gcaggaatga gcttggttgt ttgaggaacc acaagaaagc cagtgtgggc |
| 66901 | tgggcgcggt ggctcatgcc tgtaatccca gcattttggg aggctgaggc gggtggatca |
| 66961 | cctttcagga gtttgagacc agcctggcca atgtggtgaa accccgtctc tactaaaaat |
| 67021 | acaaaaatta gctgggcttg gtggtgggcg cctgtaatcc cagctagtct ggaggctggg |
| 67081 | gcaggagaaa cgcttgaacc cgggaggtgg aggttgcagt gagctgaggt gcagtaagcc |
| 67141 | attgcactcc agcctgggca acaagagtga aactccatct caaaacaaca acaacaaaaa |
| 67201 | gaaagccaat gtggctagaa tacagggagg aagggggaag agaacacaaa atcctgggag |
| 67261 | ccaggagtcc tagtgatctc taataaatgc ttaacaaata agagcttggc aacagatgag |
| 67321 | gccatggtga ggggtttgga ttttagtcca tatttggtgg gaaaaagacg tgaagagaca |
| 67381 | cacctRattt gtggKttttt tttttttttt ttttggacaa atgtataagg acctccttaa |
| 67441 | ttccctctgc ccggttccta ccttccaggt ccaccccgtg gcgctgggcc aggtgaagaa |
| 67501 | cattgtcccc gactctgcca ctcactggga tccgctggcc tgagcggtct acgaacacca |
| 67561 | cgttcaccct ggggacagat ggaagtgcga atgccgaact ggatgatggg ctcagggccc |
| 67621 | gggtagaatc tagggttagg aagtaggaat tgaggcttga cgcacaggta ctgaataggg |
| 67681 | caaagcagaa gggggacctc gagatgaagc tacagggcaa gatggggacc agaggaattc |
| 67741 | cgtgggatga ggaatggggg acttggcccc ctgcactcac acgtccccgg gccgctccgg |
| 67801 | gccgcccgcg tcctcctctc cMgccgggcg cgagcctgga aaacacggtt cggtgagcgg |
| 67861 | ctgcgccgag ccccgccccg ccggcatctg acggattaac gctgcccgcc ccgggcgccc |
| 67921 | caggtacctg tcgcttgaaa ctttctggtt gtccccagcg ccaccccctc ccccgacccg |
| 67981 | gaagtgcccc caggtctgtt ccaccaggtg cccctggcag cctgcagYag aaccctggca |
| 68041 | ctcacgcctc cccgggccat ggaggcggcc atgacatgca tcacgtgact caccgactga |
| 68101 | gcatgcgccg cgccagggag gcgagggaaa gcccataatc aatggaacat aagcgcagat |
| 68161 | gtttgcttag ggcctgcggt gaccaggtgg gaagagcagg gcggaatcca tctcaggtaa |
| 68221 | acccgcgggg ccatcccaga cctcccactc aagcccactg tttgtcccaa gaaccagcca |
| 68281 | actctgattt ctttaaaacc atttacttac aaactttaat tcagcaaagg tccgtgtggg |
| 68341 | gagactgggg tggggtcggg ggaatagtcc ccttggagtg gatgtggacc cccagagtca |
| 68401 | agggagggaa gctggtggcc cagttggctg ggggcaaggc ccagggtcac ctcaggtcga |
| 68461 | caggtcctgc tggtgggcgg gcccagagtt tatcttcatg gagtgctggt ttctggcact |
| 68521 | gggctggaag gaggccagct ccagggatct ggccgggggt gggcaggcag aattcaagaa |
| 68581 | ttcatcttca acaagcgagt gacagcagag gctccgggag atgggcacaa tgtccgactc |
| 68641 | ccacaKacag acagcagggg actggcagag aaagcccatc tctgcacgga ggcccgggta |
| 68701 | ggagggggtg gtggggccgg ttcgccaaga tgaaggcttt ccccttctac tgtccccagg |
| 68761 | gtggagatcc tgggtagggt ggcccaatcc ctaggccaga gctgtttggt ccatagtcaa |
| 68821 | gctcccagag cttggcatct gtggctctgg ccagcagggc ctggggccca gcttttaagg |
| 68881 | catcagaagg ggagggggct gcggcaggga ccccgggccc cacggctggg gtataggcca |
| 68941 | gatgggcagg caggggcagg agacagccat gcctgcagca aatgtgggaa aaacagctgt |
| 69001 | ttcaaaccgc aaggtgtgga atggtggccc ggacaggccg ggccttgaag gaatcagagc |
| 69061 | tgggggctgt ccggggtggt ttgaaaaata aaacttagaa aaggaaacag aagtcagttg |
| 69121 | tcaaagttaa aaaaaaagga gacagtctct gtatcttcac gggaggtcag ggaaacctcc |
| 69181 | aaggcactcg aaaggccaaa attacaggag caatgaggca ggagggctcc ggagagacgg |
| 69241 | gcacagcgga ggaggagatg gggggaggga gggagagcag gccggggccc ctctctctta |
| 69301 | aggctgcagg gtttcagcgt ggggagcaag ccagagacat aatgaggcct ccagactccc |
| 69361 | ccacacccct tgggctcctg gggctccggc tgattggtca gtaaagtctt tcagagattt |
| 69421 | ttctattacc gaaagagaga aaatggttta aaaaaaacac aaaacaaaac atcagaaaac |
| 69481 | ccaaaagcga tttggtgcag gcccttgagt tatctctggt gccagccact tagaaaatcc |
| 69541 | tcttccgctt caggtaggag tccgcgtagt ggccgccgag gccctgggag tgctggccca |
| 69601 | catagctgcc ttctgggctg ggctcgggcg agggcagcag gtgggcaaag ctgcgtttct |
| 69661 | ggccgcccag tggggtctgg agacagaggg cagggcgggg cgggtcaggg gccgctgggg |
| 69721 | ggccggggct tcccagccct gcatgtcccc accccctgcc cgtaccttca gcaggtggct |
| 69781 | gtgaccatta ggcccggggc tgaggcccag gagcccttct ccggacccca gcggggaaga |
| 69841 | gccgattgcc tgggagaaat ggagaggtgg aacaggtcac tccctggggg ccagaggaag |
| 69901 | cacccacccc gcccccctgc cactgccccc cagccctgag ggaaagcgaa cagtttcagg |
| 69961 | ttcaaatctt gcttctgcta ctttctagct ataagaccta gggccaattg ctccatttta |
| 70021 | tttgagcctc ggttttctca tctgtcagat ggggccagta gcacccacag cctgggctgt |
| 70081 | agagagagga agtgacagaa agcccggaat gctggcagtg ctaggtgctt gatgcagagt |
| 70141 | ggatggagga caataggctg ttgctactcc accctgagca agaccaaccc ctgccaccac |
| 70201 | cccagctttg gcctgggcac cgagggttac agagccccgg tggcaggcac ccccaaatcc |
| 70261 | tagctgtccc agggggcctg tgcttggcca gagaggttcc tcaccttact gaggtggctc |
| 70321 | tgcttgaggc cagcctgcag gccgctggtg aagtaggaag tgggcgagcc tgaatagaag |
| 70381 | tgagacaggg ggcccccgcc actcccaccg ctgctccgtt cgccgaagcc actgggcggg |
| 70441 | ggggacatct gtagaagaag ggccggtgga gtgaagcaga caattgtagt acccagccct |
| 70501 | ttgtatacaa tcttccctcY gcctcccagg ttcaagcgat tctcctgcct cagcctcccg |
| 70561 | agtagctggg attacaggca tgcgccacca tgcccagcta attttttgta tttttagtag |
| 70621 | agacggggtt tctccatgtt ggtcagactg gtctcacact cacgacctca ggtgatccgc |
| 70681 | ccacctcggc ctcccaaagt gctgggatta caggcgtgag ccaccatgcc cggcagttct |
| 70741 | gggctttgca aagactagct ttggctaata ggactgagga gagctggcac gtgggtaccc |
| 70801 | gcctcctctt cctcctccag gaacccaggg atcatcaccg cacgaatggg cacacagaaa |
| 70861 | ctgttccagc ctgtggacgc caaatatgtg aggccacata gctcactgag acccaggtga |
| 70921 | gtgccgggga ccgcccactg ccccatagag tggcaagaag taataMaatg cctggcatgg |
| 70981 | tatggtggct catgcctgtg atcctagcac tttgggaggc caaagtggga ggattgcttg |
| 71041 | agcccaggag tttgaggcca cagtgagcta tcatggagcc actgcactcc agcctgggca |
| 71101 | acacagtgag atcctgtctt aaaaaaaccc acaaagaaca cacaaaatac aaggtctgct |
| 71161 | gttccaggct actaagtcat ggcacagcct gtcatgtagt aaaagctgat tgctacacat |
| 71221 | gccataccag gtggctctta ccttgtgtcg gctgggtccg tgaggcccaa gggctggttc |
| 71281 | ccgtgggtgg gagaagagcc gccgaggtcc catgtcctga atgaaagcgg gagaagcaca |
| 71341 | ttgggttgga gtctgtccca gcctcacccc tggctgaact ccatggcaga gctgtcatca |
| 71401 | gggaagacag tccccaccac tctgggacca ggaagaggca aatttggggc tggatgcagc |
| 71461 | ttggcaggga tggctgctgt gaaggggggt gccccccatg ggcctaagtg tttggagttt |
| 71521 | cccaggcaag ctgggaattc gagctgtgtg gactcttgca gttttcctgt gttggcgtta |
| 71581 | gttcctgttg ttatttgttt gaaacacaga gggggccaaa catcacccag gtgacctgtg |
| 71641 | ggcggccagt ttgcaatttc tttctttttt ttttttaaac ggagttttgc tcttgttgcc |
| 71701 | caggctggag tgtaatggca caatcttggc tcaccacaac ctccgcctcc caggttcaag |
| 71761 | tgattctcct gcctcagcct ccctagtaac tgggattaca ggcatgtcta ccacacctgg |
| 71821 | ctaattttgt atttttagta taaaacataa attatatttt atattatata tgaaatatat |
| 71881 | attttattat atattttata atatataatt tctccatgtt ggtcaggctg gtcttgaact |
| 71941 | cttgacctca ggtgatctgc ccgcctcggc ctcccaaagt gctgggatta taggcatgag |
| 72001 | ccaccgtgcc tggcccagtt tgcaatttct gacctgccaa ggaggttggc atcatagaag |
| 72061 | gaccaaatct ccaggggcac agacaggaat caaaggtggg agagagggat agaaagggca |
| 72121 | agccctgtct ctggggagag acagtgtggc ctaagtcctc ttgggagaga aagacagacg |
| 72181 | caggatgaca gacagactga gacccaagat cctccagggc tcgagaatgg acagacaggc |
| 72241 | aggtggaccg gaagcagggg gtgatgtgca gacagacggg aaactttaag ggcatgaggc |
| 72301 | tgggcgcggt ggctcttgcc tgtaatccca gtactttggg aggctgaggc gggtggctca |
| 72361 | cttgaggtca ggagtttgag accagcctgg acaacacaat gagactccgt ctctaccaca |
| 72421 | aatttaaaaa ttagctgggc atggaggtgc Rtgcctgtgg tcccagctcc tcaggagact |
| 72481 | gaggtgtgag gatcgcttga gcccaagagg ttgaggctgc agtgagctat gatcgcacca |
| 72541 | ctgcactcca gccagggcag cagagggaga ccctgtctct aaaaaaaaaa aaaaaaaaaa |
| 72601 | gcagagacca tatctggcac gttccctgcc gcctccctag cacatggcct ggcatagtca |
| 72661 | atcctagaga aatatcagtt tggagaatga tttggcctta ggggacagag agctgcagcc |
| 72721 | aggcactcac cgaggggtag tcgaagctgt agctgtcaga cagtcctggt tcggggggca |
| 72781 | ggcgggcgct gctgaggggg ctgagcaggc gggacttgag ctggaaggct ttgctgctgc |
| 72841 | tgctgccacc tccaggggct ggggggttag ttggggggcc ctcagctggg cggcggcttc |
| 72901 | tcccggcgcc tccccagccc cgggcctcct tacccgccag gttgctggcc gggagcaggc |
| 72961 | tgtgtaggtt caggtaggga ttcaggggaa tccggtagtc cttggggggc tcccccagca |
| 73021 | gtgagacctg tggcgggaag gcaggattcg agagggcccc cgggagaggg aaccctgcgg |
| 73081 | aacaggaggc tggtgtggtg ccgcgcgcag ggcagggcct taccccaggg ggcgtcggca |
| 73141 | gaggcccact gtctttctgg aggcctctga ggccagagcc tcggagcccc acaggggcKg |
| 73201 | ggggaggagt gagctgggcc gctgggggac ccaagcccag ggcctcccgg tcacccccag |
| 73261 | cagggccaag cacggatggc agcaggggtg gtggcttccc tcgccggggc ggcagctccg |
| 73321 | ggggcaggcc ccctcctagg gagggaagga ggatttcagg ggcccaggct cacaggaccc |
| 73381 | ctctccccag ggccctccag ctcccacctg gaccagggcc caggggtcaa gagacaaatc |
| 73441 | catgctggcc actgagcaga gctcatccct gggccctgag tggagggcac ccaactgatg |
| 73501 | gacaagggcg ctgggaccca gaggttgggt cactcacccg aggccacgta gcagggaagg |
| 73561 | aaggggaagt ggctggggga caccgtgtgg gcagggtctg cgttacctgc aggcagctcc |
| 73621 | ccgaggaggg ggttggctgg ctgggcccca ggctggaggg cgcccagggg tgagtctccc |
| 73681 | aggatgccgg gcttctaggg acagagcaca gacagttgca gatgggggca gggtggccca |
| 73741 | gggaagcaga caggtgtggc ccagagtctt ggcagacaca ccccaactcc agggcacggc |
| 73801 | cgatcctggg cagacaggca cagggttccc cagtgcccgc cacactccaa ctctatcact |
| 73861 | gtcccatcag ccatggtgac cgagggtctc ctgagctcca ggccccagtt ccctggccaa |
| 73921 | tgattgcagc acagtatcaa ctgcagccac atggaatggg acagccacac agtgtttcag |
| 73981 | ccacttttag tcacagaagg ccagacccac aggctcacat attctgtcag ttaccaccag |
| 74041 | acagacacac ctgacttaga atccagggaa ccacaccaga caggacggag catgggtgta |
| 74101 | tgtctatgtg tgtgtgtgtc tgtgtgtgtg tgtgcgtgta tatctgtgtc tttgtttctg |
| 74161 | tgtgcatgag tctgtcttgt gtctatatgt gtgtctgtgt ctatgtgtgt gtctttgtct |
| 74221 | tatgtctttg tgtgtctatg tctctgtgtg tatatttgca tctttgtatg tgtgagtcta |
| 74281 | tatgtatacc tgtgtttcta ggtgtgcgtg tgtgtgtgYg cacacactga ggggatagac |
| 74341 | ctggagtttt ttcccaggca tgtctataga gcttccgagc agagtctatt cacaaactgg |
| 74401 | tgcccagcag cggcgctccc agcccacacc cagtttcagg ctgtaaatgg gcgtgtccag |
| 74461 | gtcccctgac cccaccccag gcaccaagtc caggggcgct ctcagaatgg agggaaagat |
| 74521 | ggcgaaccat catgagcaat tgccagaggg ggatggggat gtgggtggga aaggcagggg |
| 74581 | tttgggggag tcgccctcac cctggttctg cgcccccagg gcacagccag aggtcccccc |
| 74641 | accccaccga tcgcaccagc tgtgttgaaa agagcctgta ggggctgtag agcagtgggt |
| 74701 | gagggggatg cgtgtgttac cttctggccc tgggtctgca gggcgagctg caacagcgcc |
| 74761 | gtggacaggg ctggcccatt gagcagcggc atggctgggg gggcacccag gaggcctgga |
| 74821 | gggagacata ggaggatgtg tgggggtccc tgtgtcctcc ctgccccacc tcacgaaaca |
| 74881 | cagccctgaa cccattgatg gggcaggaaa ctgacactct ggggccgggg ggtgggcagt |
| 74941 | ggacttgccc aaaggaaggg cctagaaggg gcagaggcta ctccagtgaa ggtcagcccc |
| 75001 | ctcttgtcag caccccacct cactgcaggc tgctgtgtgg ccctggtccc ggcccccagt |
| 75061 | tgtggacagc agcagccccc gctatgactc catactcccc ctcggcccta ctcctggggc |
| 75121 | tcctgtcact gcagccaaga tgagcagttt cgggaagtgc cgggggcagc gctgcagccc |
| 75181 | ctgttctgca cagtggggcc cctgtccagc cccaccttac cctgcttccc ccccgcactg |
| 75241 | ccatggagca gggggttgag cagcagctgg agggacgcgg atgggcccag gttgttgagc |
| 75301 | agctgcagga tgttgggctc ggggaggagt cccttccccc gattgagggc ctgcaggaag |
| 75361 | gaacagaagc agctcagagt gccgctgggg ccctgacatc cccatggggc tcccatcccc |
| 75421 | accccgccac ctctgcccgc acaggctcac cgtggcctgg gcagcgatga gagcggccag |
| 75481 | catactgcgg ccggggggcc caggggcgca gaaggagact cgcaggtggc tgccccccag |
| 75541 | ggacaggccg tccgcctgct gctgtgcctc ctccgccatc tcagccgtct catactccag |
| 75601 | caccgcgaag cccttcagct gcccatcctg gccgcacgcc agctgccgga ggaaggcagg |
| 75661 | cagtcatgag catctgggca gcagtgactg caccaccccg ggaagtgaca accgtaacca |
| 75721 | gactcagctg ccccataagg gtgaactcca cgccaggcct ccactgccct gtgcctcatt |
| 75781 | cctgcatcag ctcatctgat tttcccaatg accctgggag gctgcatcat acccatttct |
| 75841 | cagagaaggc aactgaggca cggagaggtc agcgcaccca aggccacaca gctggaggga |
| 75901 | gggcggagct gggagccacc tcaccaacaa agccaaggcc acgggcggat gcggtggctc |
| 75961 | acacctgtaa tcccagcact ttgggaggcc gaggcgggca gataacctga ggtcgggagt |
| 76021 | tcgagaccag cctgaccaac atggagaaac cccgtctcta ctaaaaatac aaaattagcc |
| 76081 | aggcgtggtg gcacatgcct gtattcccag ctacttggga ggctgaggca gaggcaagag |
| 76141 | agtcacttga acctgtgagg cagaggttgc ggtgggccga gatcgcgcca ctgcactcca |
| 76201 | gcctgggcaa caagagcaaa actccatctc aaaaaaaaaa aaaaaaaaga gataatcagc |
| 76261 | aacgggtggg gaaggaaggg ctctcgttcc agacagtggc caagccaggt agcagtgcta |
| 76321 | agggaagtgg ttgggctggg caccggcact tgctgaatgc ccgggcagca aaggtgcagg |
| 76381 | tagcagccct tcacctctgc ccacgccctg tgccaaggtc tgggcacaga tctcccataa |
| 76441 | ccaaacctgt cggcttttgt tttgttttgt tttttttgag acagagtctt gctctgtcac |
| 76501 | cccggagtgc agtgatgtga acttagctta ccgtaacctc cacctcctgg gttcaagtga |
| 76561 | ttctcctgcc tcagcctcct ggtagctggg attacaggtg tgcaccacca cgcccagcta |
| 76621 | atttttgtat ttttagtaga aatgaggttt caccatgtcg gcaaggctgg tcttgaactc |
| 76681 | ctgacctcaa gcgatccgcc tgccttggcc tcccaaagtg ctggattaca ggcgtgagcc |
| 76741 | actgtgcccg gccataaaca cacccatctt agggatgaac agagagagac tcagaggggt |
| 76801 | cacagagtga tcaaggccac acaattccta agtcgaggga ctgatgtgtg aacctcaagc |
| 76861 | tgcttcactg tcaagcccac cctccattca gcYgggtcac ctaacattaa aggtgggctc |
| 76921 | ctcggaggac atcacttcaa cgcctgctac tcatcagatg ctcaacaaat gtttggtgag |
| 76981 | accaggccca gtggctcgaa tctataatcc cagctctttg ggaggcaaag gcaggaggat |
| 77041 | cgtttgagca caggagtttc agaccagcct gggcaacata gggagacccc atcttcacat |
| 77101 | aatttttttt ttttttgagg gagtctcgct ctgttgccca ggctggagtg cggtggcatg |
| 77161 | atcttggctc actgcaacct ccacctccct ggttcaatca attctcctgc ctcagcttcc |
| 77221 | cgagtagctg ggattaaagg cacctaccac cacgcctggc taattttttt tctattttta |
| 77281 | atagagacag ggtttttgtt ttttgttttg ttttttttga gacggagtct cgctctgcca |
| 77341 | cccagactgg aatgcagtag tgggatcttg gctcaccaca acctctgtct cccaggttca |
| 77401 | ggtgattctt ctgcctcagc ctcccaaata gctgggacca caggcacatg ccaccacacc |
| 77461 | cagctaattt ttttttttgt atttttaatg gagatagggt ttcaccatgt tagccaggat |
| 77521 | ggcctcgatc tccagacctc gtgatgcacc cacctcggcc tcccaaagtg ctgggattac |
| 77581 | aggcgtgagc caccgtgcca tgagacaggg ttttaccatg ttggccaggc tggtttcgaa |
| 77641 | ctcctgacct caagtgatct gcccgccgca gcctcccaaa gtgctgggat tacaggcatg |
| 77701 | agccactgtg cccggcctct acataaaatt ttaaaaatca gctgggcatg gtgaYgcatt |
| 77761 | cctgtagttc cccagctaca tgggaggcca aggtaggagg atttcttgaa cccaggaggt |
| 77821 | cgaggccgga gtgtactcct ccagcctggg tgattgagaa cctgtctcaa aaaaaaaaaa |
| 77881 | aattaagttt agtgaatgtt cgagtggagg aatgaatggg cagctgttca caacaacctg |
| 77941 | atcatgaagc actgccagcc ccattttgag atgtggaaac aggctaggag agtacctgta |
| 78001 | gctggtcatg accctgtgac ctttgatctc cacataagaa ggtaaggcca ggccagcgca |
| 78061 | gtggctcaca cctgtaatct cagcacttgg ggaggccgag gcaggtgtat cacctgaagt |
| 78121 | caggagttca agaccagcct ggccaacacg gcgaaacccc atctctatta aaaacacaaa |
| 78181 | aattagctgg gtgtggtgtc aggcgctgtc atcccagcta ctcgggaggc tgaagctgga |
| 78241 | gaatcacttg aacccaggag gcggaggatg cagtgagcca agatcgcgcc cctgcactcc |
| 78301 | agactgggtg acagagcaag actctgtctt aaaaaaatat aaaataaaat aaaagaaagg |
| 78361 | tgaggccatt gtgctaactc atcttctgtc ctgagcccgc acaggcggcc tggtacatag |
| 78421 | caggcctggt acgtagcagg tgtccaagag aaagcagcag aatggatgaa tgataagtaa |
| 78481 | acagagaggg ggttcccgtg gctcatggat actcagtttt tgtttttttt gtttctttga |
| 78541 | gacggagtct cgctctgtcg cccaggctgg agtgcagcgg cacgatctcc actaaccgca |
| 78601 | accgcagtct tctgggttca agcaattctc ctgtctcagc ctcctgagta gctgagatta |
| 78661 | caggcgcgtg gcgccatgcc tggctacctt tttttttttg ttgtattttt agtagagacg |
| 78721 | ggatttctcc atgtttgtca ggctggtctc aaactcctga cctcaggtga tccgtccgtc |
| 78781 | tcggcctcca aagagctggc attacaagcg tgagccactg cgcctggcct gttttttttt |
| 78841 | tttttttttt ttctgttttt gagatgcagt gttgctttgt cacccaggct ggagtacagt |
| 78901 | ggcgtgatct cagctcactg caacctccac ctcccaggta caagcgattc tcctgcctca |
| 78961 | gcctcctgag tagctgggat tacaggcctg caccaccaca cctggctaat tttttttgta |
| 79021 | tttttagtag agacagggtt tcaccatgtt ggccaggctg gtcttgaact cttgacctca |
| 79081 | agtgatccac ctgcctcggc ctctcaaagt gctgggatta catgcatgag ccacctcacc |
| 79141 | cagcctgttt cttgttttaa atatagagat ggtgtcccac tatcttgccc aggctagtct |
| 79201 | tgaactcctg gtctcgagtg atcctcctgc ctctgcctcc caacagacac ctagtttaaa |
| 79261 | tggggcacca ataccccact tacacgagag ggacccactc aagcccacag agctgggggt |
| 79321 | ccgagcccag gactgtctct ctgtttttga gatRagggtc tagctctgtt gctcaggctg |
| 79381 | tagtgtcatg gtgctgtggt gcgatcatgg ctcactgcag cctcggactc ccagaatcag |
| 79441 | gcaatcctcc cacctcagcc tcctgagtag ctgggactac aggcacacca tcatacctat |
| 79501 | ctaatttttg tatagagagg atttcaccat attgcccagg ctggtctcga actcctgggc |
| 79561 | tcgagggatc cgcccgcctc agcctcccaa agtgttaggg ttacaggtgt gagtcactgc |
| 79621 | acctggccag gcctacccct tttaagcctc tgcactggcc tccgctcact ggcccaagct |
| 79681 | ctggagctag gctgcggtgt tctgatccca gctctgccag accctggctg tttaactcta |
| 79741 | ggcaagttac ttgacccctc tgtgccttgg tatcctcacc tgaaaatggg agtgatacct |
| 79801 | gcctctcagg gttgctacag gatgaagcaa ctgaaggtta cgtactcaga gtccacagcg |
| 79861 | agtgctgggg gaatgtcccc tttgggatct ctggggatca agaccttctg ggggctctta |
| 79921 | gagagtgagt ctggcctgtt ctcgccccac tgcacctgtt acagggcgag gcacacacac |
| 79981 | agaagtgcct ggcatgtgtg gcttgaatga acaagggact gttttgccaa cccattgtgc |
| 80041 | aagtattgag tccctgctta tggaggaggg aaactgcaga tcagagagcc agtgactggc |
| 80101 | ccgagctcga gggcggggct gggatctggc aaagcctcag tctctgacct caaggctcaa |
| 80161 | atagactgtg ggctcctctg cagcctcctc aatgcctggt ccaggtctgg gaccccatgg |
| 80221 | gaggtttgac gagggtacca gcgaatgagg aggtgagtgg tgagcaagcc tcgagatcca |
| 80281 | tagttacaag agtggccttg catgcatctg atagagggac agtgaggcct gaggctgcag |
| 80341 | agggattcaa atcccctggc caggtgtggt ggctcacgca tgtaatccca gcactttggg |
| 80401 | aggatcacct gaggccagga gttcaagact atcctggcca acatggcaaa accccatctc |
| 80461 | tactaaaaat ataaaaatta tccaggcatg ggggcggggc gcctataatc ccagctactc |
| 80521 | tggaggttaa ggcaagagaa ttgcttgaac caaagaggcg gaggttgcag tgaactgaga |
| 80581 | ttgtatcact gcactccagc atgggtgaca aagtgagact gtctcaaaaa aaaaaaagaa |
| 80641 | aaggcagatg actccaaaga ctctctcagg tgctccgggc ctggcacctg ctccccaatg |
| 80701 | ctctccacag ggctcacctg gcagaaggtg gggctgtgga cagctgacag cgcccggcac |
| 80761 | agagcgtcca catcgttgaa gccaggtggc aggcggtcca cacagaggca gcgggagtgg |
| 80821 | agaagggcag gcgtcagttg cccggcatcc gtccagtgca cgtagagggt gcgtggtccc |
| 80881 | agcggcttgc ccagcaggtc Ygacttggca cgggcagccg agtccttctt catgtactcR |
| 80941 | gcaaagccat agcccttgga ttggccagtg cgctcactgt agaccaggaa gcagcgctcc |
| 81001 | aggctgccga agggccgcac cagctcctcg aactgctgct gtgtgaggct ggggggcagg |
| 81061 | ttggccacac acagcagggc atccgtgggc tgcagctgca ccgacagttc Rcgctcccgc |
| 81121 | aggcggctct ggtggaaagc attgattgcg gcctcggcct gctccccatt cagcagggtc |
| 81181 | acgaaggctg cggtgggagg agggcagtgc gaggtcagcc gggccctgag ggcctgcccc |
| 81241 | tccaccccgc caccctgcaa accaggtggg acctccaaat gctgggcatc tcaggtgcct |
| 81301 | gctgatctga gcagttgcca gccttggcga ctcacacagg cgagagcgcc tgccattgac |
| 81361 | tctaggctcc acgaagccca ctccctctcc cagccctgcg tccacctgca cccacagcag |
| 81421 | cgctcaccaa ctgtgggcct ggctctgtcc taagaacgct gtgggtcact cgggtgatac |
| 81481 | caagggcagt ggttaagccc atgctcactg gggcccagat ttaagcccca agccccaggt |
| 81541 | ggctttgggg gtatcagttt cctcatctct caaatggata taacttaatc atcacaatca |
| 81601 | ccattttgcc agagaggaaa cgggcccaga gcagcacccc tgtgccccga gtaggggagc |
| 81661 | ctggacttga acccaggcag ccggactcca gaggcccact ctacacactg cccacccctg |
| 81721 | ccacctttcc aggccaccag tgcccctgaa ccctgctgcc acgtgctgcc ccagatttgc |
| 81781 | gatccaccca gcggccgcag gaagctttgt tcaaaacgat cacgccacat ccatcttgtg |
| 81841 | gcttctagct gcagggtgca gtgcagcctg tgcgggctcg ctgacctccc ctcccatctc |
| 81901 | accgctgcca cccggaccac gccaggcaca ctccagaggg gcctttgcag ctgctgtttc |
| 81961 | ttgagcatgg aacactgagc cccacctcct cacctggtta acctggaccc acccaggtag |
| 82021 | aaaccctgtc tctactaaaa atacaaaaat tagccgggca tggtggcaca tgcctgtaat |
| 82081 | cccagctact cgggaggctg aagcagggga atgggctgaa cccaggaggc ggaggttgca |
| 82141 | gcgagccgag ctcgcgccac tgcactccag cctggcgaca gagcaagact ccatctcaaa |
| 82201 | aaaaaaaaaa aaaagaccac acaagcaggg gtgactggct gagtgaggca agaggggtaa |
| 82261 | gaagagaatg aacccacggc caaacgttgt caagaattag ctcaatgcca agcgagtgga |
| 82321 | gtaggaagag aatcagcccg atgcccagtg ttctccaagg agaacatgaa ggctgtccca |
| 82381 | agatgacagg atgacaggca gattggggca acctgccatg ttggcctggg gggcaatact |
| 82441 | gggcctgaag ctcagagcag cagacagtga gccaggccct cccatcttca gggatggtca |
| 82501 | ctccctgccc tggcccRccc catggcccca caaacctgtc cctttgtatt tgtccacaaa |
| 82561 | acagtatttg agctcatagt cactgagcag gtcatgtact tcctgtggag atacaagaca |
| 82621 | aaagacaggg agttactgga gatggaaggg ggcaacccag tgccgcttca tagctgccac |
| 82681 | cagctatgaa ggcaggtcaa ttaccacccc ccattttacc aatgaggaaa ctgaggctcg |
| 82741 | atgcagtaat tttgtcaagg tcattctgct catacctggc agagatggga ttcagaacct |
| 82801 | agctgcagac tgtgctatta cccaggcaac atgctcttag agggtatacc tttagctggc |
| 82861 | tgggcggggt ggcYcacgct tataatccca gcattttggg aggccgaggc gggtgaatca |
| 82921 | cctgaggtca ggagttcgag accagcctgg ccaacatggt gaaaccttgt ctctactaaa |
| 82981 | aatacaaaaa ttagccagga gtggtggcgc acacctgtaa tcctagctac tcgggaggct |
| 83041 | gaggtaggag aactgcttgc acccaggagg cagaggttgc agtgtgccac tgcactccag |
| 83101 | cctgggtgac aaagggagac actgtcaaca acaataacaa caaggccggg cgcggtggct |
| 83161 | cacgcctgta atcccagcac tttgggaggc cgaggcgggt ggatcacgag gtcaggagat |
| 83221 | cgagaccatc ctggctaaca aggtgaaacc ccgtctctac taaaaataca aaaaaattag |
| 83281 | ccgggcgcgg tggcaggcgc ctgtattccc agctactcgg gaggctgagg caggagaatg |
| 83341 | gcgtgaaccc gggaagcaga gcttgcagtg agccgagatt gcgccactgc agtccgcagt |
| 83401 | ccagcctggg cgacagagcg agactccgtc tcaaaaaaaa aaaaaaaaaa aaaaaaaaat |
| 83461 | aacaacaaca aaaaaaaaaa aaaaaaaaga ggccgggctc acgcctgtaa ttccagcact |
| 83521 | ttggaggctg aggcaggcgg gtcacaagat caagagatcg agaccatcct ggccaggaag |
| 83581 | gtgaaacccc atctctacta aaaatacaaa aatcagctgg ctgtggtggc gtgcacctgt |
| 83641 | agtcctagct actcgggaga ctgaggcaag acaattgctt gaacccgagg ggtgaaggtt |
| 83701 | gcagtgagca gacatcaagc cactgcactc tagcctggcg acagagtgag actccgtctc |
| 83761 | aaaaaaaaaa aaaaaaaaaa aaagagtatg cctttaggga agccatttaa cctgcatgaa |
| 83821 | cctccttttg caagattcac tgtttccaac aactctgtaa gtgaagtatt aacaaactca |
| 83881 | tgttgtatat gagaaaactt aggctaggag aagtgaagag atctgctgga ggccatgcag |
| 83941 | ttgggcagcg cagccaccca tgggtacctg actggggagc ccaccttcta gtaagcccca |
| 84001 | cgttgtgtgg gttcacatgc ctcccaattt cctcctgctg ataaagctgc cctcttccaa |
| 84061 | gccagctcat ttatttttct tttttttaga gatagtgtct tgttctgtca cctaggctag |
| 84121 | agtgtagtag cacaatcata gctcactgca gcctcaacct cctaggctca agtgatcctc |
| 84181 | ccacctcagc ctcctgcgta gctgggacta caggcacgca ccaccatgcc tggctaattt |
| 84241 | ttaaaaattt tttgtagaga tgagggtttc actataccgc ccaggctagt ctcgaaccct |
| 84301 | tgggctcaat acagcctctt gcctcagcct ctcaaagtgc tgggattaaa gggatgagcc |
| 84361 | accatgccca gcctctcaag ccagctttga aggcaaagtg ctggcgcctg ctgggaccag |
| 84421 | acacactgtg catcttgcag gagagacaat gtgttcccag ctgagagact gcagacactg |
| 84481 | agaggaagac ccagaaagaa acctgaccat ccttaatgat ccctgtcccg atctgtcccc |
| 84541 | aaattcagcc cctcccaaca tccctggggt tccactagcc acgtgcccac ccggtgtgct |
| 84601 | ggtgggccgg agagcaggga atctcagcag caggaagcga gaacctattt caccgccata |
| 84661 | gggcagggca gtatagatgc catttccctg ggtccaaaga gcattgggaa gcgctgcccc |
| 84721 | atgctggccc catgagaagg gcactcctag caggaagatt agccacgagg ggctccatag |
| 84781 | aacacaggcc tcgcttcctt cctgtaggtc tgtaggctgg tggcagaaga ccccattttc |
| 84841 | ccaaattaat cctgaggttt tgggggctga gacccagggg ctgctgttaa cttccggatt |
| 84901 | tatctctgag agcagtgaat aaacgcgtga tatggagggg cgtactctcc gcagtcctga |
| 84961 | aacttctgga tgtgaggYcc cgccaccctg tggtagagtg ggtggagaaa agcatcatcc |
| 85021 | aagcaggggg caactggtgg tccaatgtac cagcccattc acaattcccc aacaggacca |
| 85081 | aagctgtccg cccccttcca ttccccgccc gcttccatca cttggtgtcg cccaagcctg |
| 85141 | tggtcgcccc tagcaacgcc ctccctcact tcgcagtcgg tttccccttt catcatcgcc |
| 85201 | cccccacgtc ccgctcttgg tctctcccga tcccgcgtgg atccggtgct tgggcgcccc |
| 85261 | cgccaacgac ccccgcgcac ctcagcgttg gtgtcgcccg gttccccccg cgcacgcgca |
| 85321 | ccgtggtatt tcccgccacg ctcctacacc gcccccccca atacctggtt ggtcacgtcc |
| 85381 | cccgggaggc cccggatcag tatcttgcgg cggttacgga actggcgctc ggtgtgttcc |
| 85441 | aggcgtttcc ggatctcttc tggatctaga ggcggcagct cttcttccgg cgcccggcgc |
| 85501 | tccgcggcat cgccggcttc gacttcggcc ccagacttag ggctcagcgg gggccggtga |
| 85561 | gtaacggaca cgtccgccgc catcttggga aacccggcgc cttctgggac cagcgagccg |
| 85621 | gggcggagcg gcatagagcg gcaacgaggg cgcgcccgtc gattggctgg gagaaacccc |
| 85681 | acctccttcc cgcccccctt gccccgttgg ctaagcccca gcccacctct caagagttca |
| 85741 | agctgcgtct gcgcgccaca ggccccgccc ccttccgtcg cagcgacgct actcagtgga |
| 85801 | tgtgcacctg ggtgggagag acttgcagcg cgggtggaat caaaccacag attagggagt |
| 85861 | ttgaaggctt tattggtgcg gaatctgagg gcacagccaa gcccccgcca actttgatcc |
| 85921 | cggatcccag cgtcactcag ctctggacgg ttcttccccc attgcttctg tcggctgcat |
| 85981 | agacgtgagg ggcagatagg tgStctcctc cctaacatgg taactgccgc tccgttggtg |
| 86041 | ctccctgaag acgtacatta aggccagtac gatagtcacc acgcccaggg tcagtaacac |
| 86101 | cgccacgaag acggggacaa agtgggagct cccagctgtg cagagaaagc gctaagtcaa |
| 86161 | tatgcgtccc ttctgtctcc aacccccccg ccccccggct taccggtcca gacccgcagc |
| 86221 | cccgtRttag ctcaccctca atgtccatca ccacgaccag ggtgtatttg cctcgtgagc |
| 86281 | tggacgcttg gcactgataa gtaccattat gtgttacgtt gacgaagaac gggatcccca |
| 86341 | ccggcacctc ccggctggag ccttccttca aacaccgcag ctcggggtac gggttgcccc |
| 86401 | tggcttggca ctgcaggacg tgtctcgttt tatctttcca tttcaagtgc tgggggcatg |
| 86461 | tggctcggtc aattttggga ccatctgtgg aaccaccatg tgtgatcaga cacccaacac |
| 86521 | acccgaggca cagtggtgca gaggagcgtc taatctttcc agggcagggg tggagggatt |
| 86581 | aaaggtcagg gtgaccgact cacacaggac tcgcagctgg acgctactgt tcctgtgcaa |
| 86641 | gaactcgccg tccacctcga gagtggcact gcagaagaag ctgcgtccgt cgtcactctc |
| 86701 | ggtagcattt agctgaagtt gagctggctg ccccggggcc gcggccggaa ctccgtccag |
| 86761 | cgtgacctgg actcgagccc cagccatgca actcacggtc actgtggacc cctcatgggc |
| 86821 | ggtgggctcg ctgaggttca caatgggtcc taggaagcct aaaggcgggg cattgcccag |
| 86881 | gagcttaatg aacaggacct tcctgtgggt caagccgctc cctccgccct cccctttcct |
| 86941 | ctcgggatat ccgggccacg ctttcggccg ttcaagcctc gccctctttc cgcgctgtgt |
| 87001 | ccagcttcgg gcactcaggc ccaacccacg ttgcaactgc tattggggca agccaggccc |
| 87061 | caccttttcg gctagtctcc gccccctctg ccacgccccc agactgctga ggccgcgccc |
| 87121 | ccttcccacg cctcctctta ctaaagaccg tcaagttctc ccgggcctcc cgtctctcgc |
| 87181 | cccctagggt cacgttgcag acgatYtccc gggcaccctc ctgatccgcg cgcgccgtgg |
| 87241 | ctgtggctgt ggccgttagc gtgtccccgt ggttcatgac tgtcgcattc agcatctggt |
| 87301 | cccccagcgc caggtagacc tgggcctctg aggctggaaa aagcccgtct agggtgcagt |
| 87361 | ccaccggcca cgacgtttcc acctccaaga accggggggc cacgaggcgc gggggggtca |
| 87421 | cgggcaggac tggggagaaa ggtgggcata gtacaacccc caggactgtg ccttccccag |
| 87481 | gacaccctca tcccccccag tcaggatatc ttgctgactg ggtcaccctt cttcRcagga |
| 87541 | cacacacaca gggccatgaa aacggccttc ttcaaagatc ccacggctcg ggcctccctt |
| 87601 | ccggggccac cttgcccagt ggccgggggc tttccttccc agaagcctgt gaggtccggg |
| 87661 | gtaccccact ctcaggaacc ccaaggtcag ggcaccgtct accctggctc agctcgtcac |
| 87721 | ccaccgtctg aagccccttc tctcaccaaa ggttcggagc tggcgggggg ctgaggtgtt |
| 87781 | cacgaacagt cccagcccct ggggctgcat gtccagttct gtgcggcatg agaaaggggc |
| 87841 | tccgtggtcg tctctgctgg ccagcacagt ggcagtgacc tccgctggct cctccactgc |
| 87901 | gggctgccgg ctcagctcct cctcccagcg aagcagcacc accgtgaggc tggtccgggg |
| 87961 | cgacccaYcc tccacttggc agcgcagggt gaagttctgg cccaccggct gccaaggagg |
| 88021 | caggggtgcc agctccacac gctccgggag ccctgagaga ggaggggagg atggcactta |
| 88081 | gcgggtcctg caaacccacc cactcacccc agggactggg gaggagacag ggtRgtcctg |
| 88141 | ccgagaactg tgagctttga gttaataaac ttagagggct tagagctggg ccagtcgaag |
| 88201 | cgtttgctat tatcattagc gcagtgatta tcatttcctg tgttgtcaga taccttgcaa |
| 88261 | ggcgctaaac aaaactttct gttctcaaag atggcaMaat aaaaaaaaat gaggatggaa |
| 88321 | gggatgaacg tttatgacta tgatatgaat attaaaaatt cctgtttatg gccagacgtg |
| 88381 | gtggctcacg cctgtaatcc cagagctttg ggaggccggg gtgggtggat caaRaggtca |
| 88441 | ggattattca gttctctcca atcaggttca gacactcagg gctttcctgg ttcaccagcc |
| 88501 | ctgtggcagc ctctcaagat actccggcac agtcacagga gacttgacac tcccaggctg |
| 88561 | ggtgccctcc ccagtacacc tacaaaacgc tgggcctcag gccgggcact gtggctcatg |
| 88621 | cctgtaatcc cagcactttg ggaggccaag gtggacggat cacctgagac caggagttca |
| 88681 | agaccagcct tgccaacatg gtgaaacccc atctctacta aaaatacaaa aattagccag |
| 88741 | gcatggtagc acgtgcctgt aatcccagct actcaggagg ctgaggcaca agaatcgctt |
| 88801 | gaacccagga ggcaaaattt gcagtgagct gagatagtgc cactgcactc cagcctggcg |
| 88861 | acagagagag actctatgtc aaaaaaaaaa aaaaaaaaaa aaaggctggg caaggtgact |
| 88921 | catgcctgta atcccaccac tttgggaggc cgagggaggc ggatcacttg aggtcaggag |
| 88981 | tttgagacca acctggccca tatggcaaaa ccctgtctct actaaaaata caaaaattag |
| 89041 | ccgggcatgg tgtcacacgc ctgtaatccc agctactaag gaggctaaga caggagaatc |
| 89101 | acttgaaccc aggaggcgga ggttgcaatg agctgacatc gcgccattgt actccagcat |
| 89161 | gggggacaat agcaagactg cgtctcaaag aaaagaaaag tcaaaaagta ggccaggcat |
| 89221 | ggtggctcac gcttataatc ccagcacttt gggaggccaa ggcgggcaga tcatttgagg |
| 89281 | ccaggagttt gagaccagcc tgggcaacat ggtgaaatcc tgtccctact aaaaatacaa |
| 89341 | aaattagctg ggcgtgttgg catgcgcctg taatcccagc tactcaggag gctgaggcag |
| 89401 | gagaatcgct tgaacccgga aggcagaggt tgcagtgagc caagattgcg ccactgcact |
| 89461 | ctagcctggg ggacagagtg agactctgtc tcaaaaaaaa aaaaaaagaa aagaaaagaa |
| 89521 | aaaaaaaagt agccaggcct gcaatcccag ctactggaga gactgaagtg ggaggattgc |
| 89581 | ctgagcccag gagtttgaga ccaacctagg caacataggg agatcctgtc tctaaaataa |
| 89641 | aggtataaaa aagtgtaaat gtaaaacctc catttgccta ctgctgactg aaaggggtac |
| 89701 | cccctttttt gcttaagaga caagggtctt gcttttttgc acaggctgga gtgcagtggt |
| 89761 | gcaattatag ctcactacag ccttgaactc ctgggctcaa gcgattctcc caccttggtc |
| 89821 | ccccaagtag ctgggacttc aggcatgtgc caccatgccc agctaatatt ttttatttat |
| 89881 | ttttgtagag acggggtgtc cctatattgc tctgcctggt cttaaatttc cggactcaag |
| 89941 | caatcctcct gccttggcct cccaaagtgt tgggattatt gttggggcca ggcgcagtgg |
| 90001 | ctcacacttg taatcccagc actttgagag gctgatgtgg gcggaacacc ccgtctctac |
| 90061 | taaaaataca aaacttagct gggcattagc tggacctggg aggcagaggt cgcagtgagc |
| 90121 | cgagattgca ccactgcact ccagcctggg ctacagagta agagacgcca tctcaaaaaa |
| 90181 | aaaaaaaagc ttgttggggc cgggcgcagt ggcccatgcc tgtaatccca gcactttggg |
| 90241 | aggctgaggt gggcggatcg cctgaggtca ggagttcaag accagactgg ccaacatggt |
| 90301 | gaaatgccat ctcaactaaa aatacaaaat ttgctgggtg tggtggcgtg catgtgtaat |
| 90361 | ctcagccact cgggaggctg aggcaggaga atcacttgaa cccaggaggc gaaggttgca |
| 90421 | gtgagccgac atcgtgccac tgcactccag cctaggcaaa aagagcgaaa ctctgtctca |
| 90481 | aaaaaaaaaa aaaaatgctt gttggggccc aagttaattg atttcacaac tgctgccttg |
| 90541 | gcatgagatt gagatgcgtc tctatatctc tgtctctgtc tctgtgtctc tgtctctgtc |
| 90601 | tRtctttgta tctcacacac aaatgtccag gtctgagctc tgggtcagaa ctgtggtcca |
| 90661 | ttctttgcaa actgagggtc cccactctcc tgagccattg gccacttgcc cacaccacta |
| 90721 | cccaagacca gtcccacctc cggacacgtY gactcactgt acacggtgat gttagaggag |
| 90781 | cctgttatct gRgagccatt gcagtacact gagcagagga tccgactgtt gccagtcacg |
| 90841 | ttgctgagat tgaaggctgc ccagcccatg ccactggcca ccagctcctt tgatagggac |
| 90901 | gtctccaagg cgattttctc agagctggga caatcagtac tgcagttcac aaacagggac |
| 90961 | cctccagcag agagcacagg gttctggggc tccacccgca aaaggaactc ctgcccctgg |
| 91021 | acaccttcag gaacatgaag aagtcctggt gtttgtttcc ttgtYcccca acccacgcat |
| 91081 | caacaggccc caccatttaa cacctctctc cttgtgccga gacagaaggg ttcctgcctt |
| 91141 | catggtccag Kgggaaaggt agaatccatc agagaccRgg gagaactcag aaggaggcca |
| 91201 | gaagaggctc tgatccagca taggaggagg tgagtcaggg aaggcttcct ggaagcgggg |
| 91261 | accattgcag cccgaaactg aaggctaagt aggagttagc aagtgaaagg gacaggtgat |
| 91321 | cctggcagag ggaagcacat acgcaaaggg caaatttcag gagttcagat ggagcaggaa |
| 91381 | gcatgagggg tgtgtgtgtg tgtgttaggg gagagggatg gcgtacaagc ctatgctggg |
| 91441 | gcctgaccac ctgaaggctg aactaaggaa ccagaacttt ctcccgagga tacagagtca |
| 91501 | gcggccaggt cgcagtgtgg caggatgtga acagcgttgc gttctattcc tctaccctct |
| 91561 | actgatcccc aaaacgcagc cctctccagc cctccccggc ttgactggtc tcacctgggg |
| 91621 | tcagcagaca gcagaccagc agagtccagc aggccctggg ccacaacacg gatggtacca |
| 91681 | tggtRgccat tctgacagag gaaggtgcct tcctgaggtg cccgaagggc aggcagggga |
| 91741 | aaaggcgggg cctcccactg cgacggaagc acagcggtta agattgcaga agtctgggga |
| 91801 | ctgcaccttg cccaggcctg tgggtggaag gggatgctgt ggcccttggg ggccagggtg |
| 91861 | ggggacccag acggggctct gcaaagtggc cagcactgct gagtctcccc cagggaatgg |
| 91921 | gctggtccca gttgcactgc tggcccccat ttgaattcac aggaaatgct aactaaactg |
| 91981 | gtttcttttg ttctcagcac tgggaagcct gggtgggggg gatgaggcat ctccttctgg |
| 92041 | ctgaacttag gtgggtaggg gaatattctg tccccagaga ggaagttaat ggggaaaact |
| 92101 | gtaggcacca ttgtgtagat gttaaggtgg gatttcgagg aaacattaaa aaacaaaaac |
| 92161 | aaaaacagaa acagaagcag agtcttgcta tgttgccccg actggtcttg aactcctggc |
| 92221 | cttaagatgg cctcctctca tcttgacctc ccaaagtgtt gcgattacag gcgtgagcca |
| 92281 | ccacgcctgg ccacctttga agatcttgag gatacctccc tatctttgaa gatcttgggg |
| 92341 | atcccccatc ccctgcacaa agagctaagg taggtgattt ggggacagct gaKtccctga |
| 92401 | gccactgtct atccaggatt ctacttggtt tttgtttgtt tgttttgaga cagagtctca |
| 92461 | ctctgtcacc caggttggag tgcagtggcg ggatctcggc tcactgcagc ctctgcctcc |
| 92521 | cgggttcaaa cgattttcct gcctcagcct cccgagtagc tgggattaca ggcatgtgcc |
| 92581 | accacgcccg gctaattttt |

Following is an ICAM genomic nucleotide sequence that includes the ICAM SNP “ICAM-AA” (SEQ ID NO: 4938).

CCGTACCCTAGTTCGTTCTCAGCACCCCGAGGATTCGGGCAGATAAGG
GGCGGGCCTTGACCGGAGGGAGGGGTATGGTCAGTATACTACGACCAA
ATGCTCCGCCCCCAGGCTTCCCGGCACCACTCCTGACCCTGAGCGAAC
CCAGCGTCTCCGAGGGGCAGATGGTGACAGTAACCTGCGCAGCTGGGG
CCCAAGCTCTGGTCACACTGGAGGGAGTTCCAGCCGCGGTCCCGGGGC
AGCCCGCCCAGCTTCAGCTAAATGCCACCGAGAACGACGACAGACGCA
GCTTCTTCTGCGACGCCACCCTCGATGTGGACGGGGAGACCCTGATCAAG
AACAGGAGCGCAGAGCTTCGTGTCCTATGTGAGTTGGTGATAACCCCTCG
CCCCCCACCTTCTGGTGACTTCCAAGGACCCGCCTGCT[C/T]CCTCACC
GTGTCGTGGAGGCGGAGCCATTTCTTACGTCTAAGCCTCTGTAACCCCAC

Following is an ICAM genomic nucleotide sequence that includes the ICAM SNP “ICAM-AB” (SEQ ID NO: 4939).

GGGAAGGAGGCTCTGATAGGAGGCAGGATCCTCTTCTGCCCATCAGAGG
CGCGGTGGTCTCCCATCGATCGTTGTGGGCCGGAGCAGGGCATTTGATCA
GTGGCTGGGCCGGCGCTAAGCCCCACTTCACCTTCTGTGCCCTTCAGACC
GGCCAGTGGTGGCCGAACTTGCTGCCTCGCCCCCTGGAGGCGTGCGCCC
AGGAGGAAACTTCACGTTGACCTGCCGCGC[G/A]GAGGCCTGGCCTCCA
GCCCAGATCAGCTGGCGCGCGCCCCCGGGGGCCCTCAACATCGGCCTGTC
GAGCAACAACAGCACACTGAGCGTGGCAGGCGCCATGGGAAGCCACGGC
GGCGAGTACGAGTGCGCAGCCACCAACGCGCACGGGCGCCACGCGCGGC
GCATCACGGTGCGCGTGGCCGGTAAGTGGCAGCTGGGGAGAGGCGGGGCG
AGGTATCTGAGAGGGGGCGTGACCTGGGTCTTGGGGCGGCCGGCCCCGC
CTGCCTCCCTCTCGGTCCCGGTAGACTAG

Following is a MAPK10 genomic nucleotide sequence (SEQ ID NO: 2).

| >4:87372101-87483050 | |
|---|---|
| 1 | attttcccaa aatactagac agtcatatat aatggaaact gcaaacctcc gaaagtgtgt |
| 61 | ctgcgtgtgg gcaaatagta aaatagcatt gaatacacaa tgtcttcaaa cacagtacac |
| 121 | aagtttgaca gttattctaa ctgctctaaa attcttttta tcagattcac tgctaacatg |
| 181 | catggaaaat atgatgacaa attcgRagat tcctaaaaga gaaaagaagg ctatataata |
| 241 | caatcaatta ttacattgtc ttttttcaca attctgtaat aattgatggt aattcaatag |
| 301 | atatttatct ttatctaact ttatctttaa aactatccca agtttactta ttactgctac |
| 361 | ggttttaaat tgcagcagct gaaatatagt aggacaaatg cataatttgg aataagacta |
| 421 | aatattgcat ttcaccttga tcacatcgtc tctgtgccct gagcaaatag tttaacttct |
| 481 | ctaaccctca attttctcat ctgaaaaaaa aaatgaatcc tctctttcgg actttcaaga |
| 541 | aatttaattt ggaagatata agggaaacat ttgcatctac tacagtgttt ctcaaagtgt |
| 601 | aatgcaccca ggaatcacct ggggattctg attcagaagc tctgcattgg gtgctaaaat |
| 661 | tttcagcttc caggtgatgc cagtgctact attctattat tgctgatgtt atctggaagc |
| 721 | tggtttataa tgcaaaatct catgtcccac ccaatgccta ctgaatttca atcttccttt |
| 781 | taacaggatc tccaggtaat ttttatgaac tttaaaattt gaataacatt gatgtagcag |
| 841 | tagatcttgc ttattgctga tatgcagtcg aaaatattat aagttttgaa tcatcacaag |
| 901 | aaaagtgtgt agaatataaa tgaatataat atgttttttc ctcttcaaaa tctgattttg |
| 961 | aattatattt cccaggtgtc accggaagct aaagagtggc cttgcctcag gtcaggtttc |
| 1021 | tactccctga cagcctctca attagctgct gttgtaatga aattatgcat tgtcaaaagc |
| 1081 | ctttttgcat cattttcttc accaaatctc aagttttaag tattttatat agaaactaca |
| 1141 | taaaatccaa ccctgctctg cccattccag tggctgccag atagctggtt ttctttgtga |
| 1201 | ttttgggctg ttggttttct aggagaggag gagaagatta gagcaagtta aaacgccaca |
| 1261 | gaccttgctg ctcttactga gattcagcca tttttcttaa gtaaacactc tccagattgc |
| 1321 | atcaagccat tagttaattt acagaattcc aaaatagttg attctggaga tttttgctag |
| 1381 | ttgcttatat ggtgaagagg atttttggag gtcttcacta caccattctg gctgatgtcg |
| 1441 | cttaaaatgc cctgaagttt ttgaatggtg aacaaaagtc ttatggattt tcattatctt |
| 1501 | gataYattag agaaaccatg catccacctg attgtattgg gctccattta cagatgccac |
| 1561 | taaaatatct tgatagtttg aggcaattat tagatgagag gtcacagtga cctcctccta |
| 1621 | agaaatattt ctcagaatta aatgtcaaat ttttaataaa cattgtatta tgattaagag |
| 1681 | cccaggtttt taatgaaccc tgggatcaag ccctgggtct ggtccttaac gtgtaacctt |
| 1741 | aatcaagtta cttaaccttt ataatacagt gataataaca gtatgtatgt atagatttat |
| 1801 | ttcaaaattc aattaaatta caaaagcaaa gtgcttgaca cagttcatgg cacaaagtaa |
| 1861 | atgctcaaga tgttttagct tctattgcta ttatcatagt taagaatatg gtttggtact |
| 1921 | gataataata atgaatgttc tcttgaatgt gtttctttaa gagtcacatt gaaatagaaa |
| 1981 | taaataaaaa ctgtctcact catgctactt tatgttctat gcaattctag gcactgtggt |
| 2041 | ggtccagcag ttgatgtcag tgatgattag caaacatgat tgcctctttc ccaaagatgc |
| 2101 | agaactacaa agcaagcccc aagatggagt gagcaacaac aatgaaattc agaagaaagc |
| 2161 | caccatgggg cagttacaga acaaggagaa caataacacc aaggacagcc ctagtaggca |
| 2221 | gtgctcctgg gacaagtctg agtcacccca gagaagcagc atgaacaatg gatcccccac |
| 2281 | agctctatca ggcagcaaaa ccaacagccc aaagaacagt gttcacaagc tagatgtgtc |
| 2341 | tagaagcccc cctctcatgg tcaaaaagaa cccagccttt aataagggta gtgggatagt |
| 2401 | taccaatggg tccttcagca gcagtaatgc agaaggtctt gagaaaaccc aaaccacccc |
| 2461 | caatgggagc ctacaggcca gaaggagctc ttcactgaag gtatctggta ccaaaatggg |
| 2521 | cacgcacagt gtacagaatg gaacggtgcg catgggcatt ttgaacagcg acacactcgg |
| 2581 | gaaccccaca aatgttcgaa acatgagctg gctgccaaat ggctatgtga ccctgaggga |
| 2641 | taacaagcag aaagaacaag ctggagagtt aggccagcac aacagactgt ccacctatga |
| 2701 | taatgtccat caacagttct ccatgatgaa ccttgatgac aagcagagca ttgacagtgc |
| 2761 | tacctggtcc acttcctcct gtgaaatctc cctccctgag aactccaact cctgtcgctc |
| 2821 | ttctaccacc acctgcccag agcaagactt ttttgggggg aactttgagg accctgtttt |
| 2881 | ggatgggccc ccgcaggacg acctttccca ccccagggac tatgaaagca aaagtgacca |
| 2941 | caggagtgtg ggaggtcgaa gtagtcgtgc caccagtagc agtgacaaca gtgagacatt |
| 3001 | tgtgggcaac agcagcagca accacagtgc actgcacagt ttagtttcca gcctgaaaca |
| 3061 | ggaaatgacc aaacagaaga tagagtatga gtccaggata aagaggtaag gaaaatctgg |
| 3121 | aggtcgtaac taccagagag ggctcagtag cctcctactg tgggacagga tcacactgag |
| 3181 | ggtgacatat gctggctcca aagtcaaaga aacacaagtt tgctccatca tttatgagct |
| 3241 | ttgtgactgt gtgcaagtta gcttcatctc tgtgagcttt gattccctaa tgtaaacaat |
| 3301 | ttaaatacta gtagtatcta cctcaaaagc acataagaag tgattgaaca tttccctggt |
| 3361 | acagaatagg ctcaataatg tcatctttga ttattattaa atagatagat ttgcattaga |
| 3421 | gggctggaag gagaggggct gtgaggagat ggacagagca ttaggagcct gttagttttc |
| 3481 | actaacaaga aaaggatgaa actagggtaa aaaaagtaga agtaaataga cctccatgtt |
| 3541 | ttattctttc aatcattgaa tgttgagaga gtgccttcta tctgccaggc tctgttccag |
| 3601 | acgctgagga ttcagatgag ctgagagagg ccataagagc caggtgacat aggggattga |
| 3661 | caggctgatt ttcagccttt gacttttact ctgagataga aatccatgga ggtttttgag |
| 3721 | aagaggggtg acgtgaagta tgttcacatg aataaggaag actgtagcct aaatgaggaa |
| 3781 | aaaagaatat gatttRtaaa gaagcaatta tgctaaaacc taattatatc aatgtgcatc |
| 3841 | ttctcataaa atgatcaaag taggagtagc tcctctagtg ttctcagtcc cagtttcagg |
| 3901 | ctgtgtgatt tggacaaccc ttcctccatg gtgtgctagg atcctcagtY ggttgttaag |
| 3961 | ttggctttaa tttaccttaa tatgtttcct atccattcat caattatcag acaaaaagat |
| 4021 | aacgtacttt cagaagctat ggcaaaataa atattaaaaa atactactta taaatgagat |
| 4081 | tgaaaaatta cttccttgga aagtattttt ttggtaagtt tcactgagtg gaattgtggt |
| 4141 | ttgaattttt ctatataatg gagagtgaca ggagatttaa agaaaaatat agggtcatta |
| 4201 | tcttaaaagg ttactcttct ttctgaatgt agtttttgga accactatgt attggaattc |
| 4261 | tgaattactg ccaatccatg atgataaatt agcatttcac actgggaagc ttatattttg |
| 4321 | aggcaagggg tcacacttta aatgtagtag aaaaagaaaa aagttatcac tatgaatttg |
| 4381 | cagatggata cattcatttc tgaggatgtt gctgatgcat atatgttctg agatctacgg |
| 4441 | tatatttcag agattttgat aaactgatag aacttcccaa tagaggtcat cttaatgggc |
| 4501 | catttcagtg tcacattctt accctcRtga caggccaagg gagagctctg cagtagggtg |
| 4561 | gagggtgggg gaccgctcgt ggcctagcca tgaaattata gaaagttaac tgttctggct |
| 4621 | aaattttgat catttctgtt tagagttatt catttttctt taaattcctt tataacttta |
| 4681 | ccccacaagc attgaaggta gatggatgga tagataggta ggtaggtaaa taggtaggta |
| 4741 | ggtaggtagg tagatgatag atagatagat agatgataga tagatagata gagagataga |
| 4801 | taatagatag atgatagata tagatagata gatagataga tagatagata gatagataga |
| 4861 | taatatttta ctttgtttaa ttatcaataa gatatatgtt taaatcattt ataactttac |
| 4921 | cccacaagta ttgagggtgg atggatggat aggtaggtag gtaaataggt agggaggtag |
| 4981 | gtagacagat agataatatt ttactttaat tatcagtcag tgagatatat ggtttttagt |
| 5041 | tttgaaacat atattctttt tccctctcca aattggtttc taactttttc ttttcttatt |
| 5101 | tttttttatg ttttattttt tgacagagtc tcactctgtt gcccacgctg gagagtgcag |
| 5161 | tgatgcaatc ttggctcact gcaacctctg cctcccaagt tgaagcgatt ctcctgcctc |
| 5221 | agcctcctga gtagcttgga ttacagatgc atgccatcat gactggataa tttttttttt |
| 5281 | tcttgtggag acaggggttt taccatgttg gccaggcagc tcttgagctc ttgacctcaa |
| 5341 | atgatccacc cacctcggcc tcccaaagtg ctggggttac aggcataagc catggcactc |
| 5401 | agctagtttc taacaatttt cttaggaaat attgtattgt ggtcagagaa gttcatctgg |
| 5461 | atgatattaa ttatttggtc tattaaaact attcccttat tactggattg ggattctata |
| 5521 | tatgaccaag agatcaagtc tgttaatcag gctattcaaa gcttctgtat ttcttctcat |
| 5581 | ttttatcagc tatatcaata atttttgaga aagatacttt aaaatttcct atcagtattc |
| 5641 | tgagtttatc agtttctttt tataattttg tcatatttta tattgaacaa tttgaggctc |
| 5701 | tactactagg tgaatagaag tttggagttg ttcatatctc cctgaagaat tgatcctttt |
| 5761 | atcattaatt agtgcctatt tttcattgta ataatgcttt ttacctgaaa ctctattttt |
| 5821 | tctgatatta atattgtcct accatcttcc tttgattagt aacttcctga tatgtctttt |
| 5881 | cctttattta actttcaaac tttgtttgat gatttgatct aacaacttac atctttacaa |
| 5941 | ttctggaata tttttttcat catctttttg tctatttcat cttcccacct tctcaatttt |
| 6001 | ctctttccag aacccttatt aagctatgtt tgaccttgtt aatctatcct ctgtctttgt |
| 6061 | taacttcttt tttccatatt aaatttctat aattatcatt tttgaggcat tctaggtaac |
| 6121 | ttttctcaga cctgttttgt agcttactaa tactctgatg agctaattca attctgatac |
| 6181 | ttccccaggc ccatgattgg agattttgta gtctcttctt cataaaaaca gttaatgttc |
| 6241 | caagctctca gttaaattac ttgtagtttt ctgcctgcct aatactggag gcctcacctt |
| 6301 | ctgtccccat gtgaaaaaga aaaacctaga cccaagctct gtggcctcta tccaagacta |
| 6361 | atatccttcg tgtgcttcta cttattgttc tatatttcgg tttcttcctt ggttctgatg |
| 6421 | gctgaagctg ctgttttatt ttaaccttga tgatgtattt taaaaatata tttcttaaaa |
| 6481 | cttttccagc acttttatat gtttgcattg ggagggattt cttctgtatt caacttagtc |
| 6541 | cttttcattc ctggaagttt cccacaagca cctgatacat acaaatgtgt caatatattg |
| 6601 | ttttgtaagg gttgtccttc tcagtatttt aatttcaatt tcttccaaaa ttttggctct |
| 6661 | aaaatagctt tggaaaacaa agagaatata ttgtagaata attctgagtg gaaaccagca |
| 6721 | tagtaccaac ttttcatgta aaaatgtggt catatgttta aaatagcctt ctaaatgatg |
| 6781 | taggattaca aaatatgaag tattattttt taaaattcta gttgcttaca aagagaagga |
| 6841 | agattaattt ttaagtgttc ttattatctg gatgaaatat caacatgtgg ggatcaaatt |
| 6901 | tttcaaagca gaaacataat tgagatttta atttaaaaac tgttattaaa ataatcagat |
| 6961 | atctgagagc tacacatgca ttaacctttc aaatttcagt ggtatggtaa gtttgaatga |
| 7021 | gtcccatggg acttttgctc agaataggag aaattaaaaa gtcagccgag gccaagatac |
| 7081 | atcttaaata aagatgtaac tttaataccc ctcttttgaa taatatatgt atatgatttg |
| 7141 | gatcttagtg gttatggaga aaacagtctc tgtttttaaa aacgttactg cgaattctac |
| 7201 | aattatccaa aagctataca aaacaactat caatttatag agatataaaa tacctatgcc |
| 7261 | cagagctata ttaataactt gggaagcagt gagaattggg gatggaaagg aatgggcttt |
| 7321 | atttttacaa aatatgagat gcagtgctta tttaacacat gtgatcttta cctctcgtat |
| 7381 | caatgtaagc tcattacttt gggtactctt ttataagatg ggactctata tgctgccaaa |
| 7441 | aaccccattc ttagcagtga acttaaccaa caatgacatt ttcctatcac tatttttcct |
| 7501 | tttatagaag gcaaatgtta ttgccctctt tttttttttt ttttgttaac tatcagactt |
| 7561 | tcctgctgca tttagtgtag tgaacttYac aatttaatgt ccaggaagct ttctgcctcg |
| 7621 | tctgcccggc atgtgttact ggcttatctg tgctggcatt cacttattta ctattaaatg |
| 7681 | acagctgctt ccaagagagg ccttgggacc taatgactaa ggtgacagta aatcatctta |
| 7741 | gcaaacagta aaataataca acaaaattac atcctttaaa tcattgaaag ggctttattc |
| 7801 | caatttccta agcatcataa agagtttaga aaatatttgt atgtctcttg catttcaagt |
| 7861 | gaataaaatc actttatctc ttactcttgc gtccccaccc cccacccccc ccaacatcct |
| 7921 | ttgtagctta gaacagcgaa acttgacttt ggaaacagaa atgatgagcc tccatgatga |
| 7981 | actggatcag gagaggaaaa agttcacaat gatagaaata aaaatgcgaa atgccgagcg |
| 8041 | agcaaaagaa gatgccgaga aaagaaatga catgctacag aaagaaatgg agcagttttt |
| 8101 | ttccacgttt ggagaactga cagtggaacc caggagaacc gagagaggaa acacaatatg |
| 8161 | gattcagtga gcctgctttc gcctgctgtc tctgatggct ctggcaagga ctccagggat |
| 8221 | tctggtggga tatgacttag aaccaggtgg ctggtcacct ggatgtacag aagtctaact |
| 8281 | ggtgaaggaa tatcatttac agacattaaa catccatatc tgcaatgtgt accaaagtta |
| 8341 | tatcatgccc cataatgcta ctgtcaagtg ttacaactgg atatgtgtat atagagtagt |
| 8401 | ttttcaaaag taaactaaaa atgagaagca tatttcaaga attattttat tgcaagtctt |
| 8461 | gtatttaaat gttaaatcaa tWtgttgttg caatttagct tgctttcaag cttcacccct |
| 8521 | tgcacttaac ataagctatt tttggcattg tgttatcatc ggcttatttt atagatcaat |
| 8581 | atttttattt cccttttttg ctgaggaaat gaagataagc aaaaatataa atatatatat |
| 8641 | aaatatatga gttattaaaa tcagaagaat actttgtggc tgtgctgttt gtgccaatag |
| 8701 | actttgtcat gaccaaaaag agaaatgtaa atagttttat aaaatacagt cgaatcacca |
| 8761 | ggaacctttg agctgctttt aaaattcttc ccctggcacc actcagtttt gcttttgcga |
| 8821 | ggcgatttga cataggaact ttgagactcc atgagaaagt ccctttctga ggcccactgt |
| 8881 | ctaccttgcc agatcctcag tgcgtatcgc caatgcagga tgctccttag aaaagaaaaa |
| 8941 | atggtaaaga atggcattta acgattcagg ctttgaatta ctctgtccct ctggaccgaa |
| 9001 | tctctttaac tgctgSaYag ttttagagga attctcctgc tacttaggta ctgggaaaca |
| 9061 | atgcttgcta aaccatgccc acgtgagcac ctgtctccca ctcaaacctc tcccatctcc |
| 9121 | caacaactgc actttagaat accagcagtg aaatggtatt actgtttccc tctgagtgaa |
| 9181 | actgctagag tatatgtcac gtagtgacat ttttttctca ctcaggctat tgccatctgg |
| 9241 | gattctctcc ctactacagc tggcaaagtt ggtttgcagc aagaagatag tgggaggggg |
| 9301 | ccaggctgca ggagaaggag aaaagtttag aagaaacaaa ccattttgct tctaattttg |
| 9361 | acagtatcac tttcctgtta aaacatacaa taattttaaa aggtgaatgc ctaaagttcc |
| 9421 | aattttagca aatatgggaa cctcagcaat gctaattttc tagaaaaacc cagggctctt |
| 9481 | tggagctaga gttttgggag aacagttctt cacaataagg caatggtttt gagaggccag |
| 9541 | gcaaataatc tttctcaccg tagaacaaaa agttacaaaa ggcataatcg gaaatagaga |
| 9601 | ctacatactt gagtttatgg ggtttgtgtt gtttgaaggt tcaatgcttg catgtgttta |
| 9661 | tttattttca agagggaaag tggtctgtac tgctttcatc cttgccactg tcttgctttt |
| 9721 | attttttact ctcccactga gcaagcRtct gtggtcctat ggtatcaacc agtatcttta |
| 9781 | tagcaataat ttctttaatt cccttttctc tctctttcca attatttaac cagttacttc |
| 9841 | cacctggaca tacgatagga aattcaaact caaaatatga aaattgatct taataactct |
| 9901 | cccttcatat cttttcacct atttccagtc cttatcatag ttgataaaaa cctcagactc |
| 9961 | atccagaaag ctatatgatg cactagtaaa aaaaacaaag atattcaaac tgcttgggtt |
| 10021 | caaatggtat acaatttgcc agctgttact gaaccttcta tgcataactt tttttttcct |
| 10081 | ctgtgcaatt ggaataataa aaatactact cccatagagt tgttatgtgg tcttaatgag |
| 10141 | atagcttggg ttaggctgag cacagctgct agtagaatcc aaaaaggggc caggcatggt |
| 10201 | cgctcacgcc tgtaatccca gcactttggg aggccgaggc gggcggatca cgaggtcagg |
| 10261 | agactcagac catcctggct aacacagtga aaccctgtct ctactaaaaa aaaatatata |
| 10321 | tatatatata tacaaaaaat tagccgggtg tggtggtggg cgcctgtagt cccagctact |
| 10381 | cgggaggctg aggcaggaga atggcgtgaa cccgggaggc agagtttgca gtgagccaag |
| 10441 | atcaagccac tgcactccag cagcctgggc gacagagcga gactccgtct aaaaaaaaaa |
| 10501 | aaaaaaaaaa aaaaagactc cataaatgtt aacgcatatt ttctttcttt ctttctttct |
| 10561 | ttttttttgt ggggggacag tctcactgtc tccttctgtc acccagtctg gagtgcagtg |
| 10621 | gcatgatcac ggttcactgc agccttgacc tccctaggtt caggtgatcc tcccacctca |
| 10681 | gcctcccgag tagctgggac tgcaggcata cgccaccatg cccagctaat ttttgtattt |
| 10741 | tcttttgtag agacagggtt tcgccatatt gcccaggctg gtctcaaaca cctgggctga |
| 10801 | agctgtctgc cctcctcagc ctcccaaaag ttatgcaaga tgcatagcaa tgacgggtca |
| 10861 | cttaatttgt ctgggactac aagcgtgagc caccgcgccc agccaactca tatttttctt |
| 10921 | ctctacccac ttcttcactc ataaatttgc tcaccagcta atctattcca ctcatctttg |
| 10981 | cctttttaat ctgctctctt taatcacttt tcccttacct tttgatgaag gcatcctacc |
| 11041 | cagcgcgttt tgttccagca tctctcctgt acttttgtcc tttgcaggct gccttttcga |
| 11101 | aattattcat ctgaccatag cttgatcttg ctcattacat atgatgactg tcacttgccc |
| 11161 | aggacaaagc ccttcacata gtgtaaaagg tcctactgcc agtttcattc ttgtctccta |
| 11221 | ccacttccca ccacacctgc catgccattt catacctcta gaactttcca cttgctattg |
| 11281 | ttgtgtcctt cttgcttgcc ttctttctag ctggcatgct ccaaacatcc tttaagagcc |
| 11341 | aaatcaatgt cagtttcctc atgaagcttc ccagaatctc aaatcagtgt tagttgcttt |
| 11401 | ttgtgccccc attcgcatta tctctttctg tctttcccac atctgattat tttctttatc |
| 11461 | aaataagaag caatttagga gggagagtga tgaagtggaa atagcagaaa attgagagcc |
| 11521 | attcaaacct agatctgcct ctcaacatgc cttatgcaag atacatggca atgccaggta |
| 11581 | acttgacttg tctgatctca tctacaaggt ggagataata actcctacaa agtagggatg |
| 11641 | ttgccgtggt taatggtggt atgtaatgtg cttatcccgg tgcctgtcac tttgctccac |
| 11701 | tgataactct attgataaat ggagcttctt aaggggccaa gattgtatct tcttgctgtt |
| 11761 | aaatccctcc atgccttatg ccaaagagaa tgtcagcaca gtgagtttgc agtaattttt |
| 11821 | gggaatgcaa atatactcaa acacacacac acacacaggg tgttttgtgg cctgtgatat |
| 11881 | ttgagggatg atattatcaa ttttaattgt caatttcagt gatgatggca ctaatcaatg |
| 11941 | gataccatga gcagcatgtt catctgggtt tccgttagtc aaataaaaag cctagacaat |
| 12001 | attaagtaca attaacataa gaataaattg gaaatcaata aggatacaaa gtagttaggt |
| 12061 | agtatcaaga tttcatttgc tacatcctgt atatgcctct ttaaaactat ttaggtaatt |
| 12121 | aaaatgaatc ttaagttata aactcgagaa aggatttgct ttcagatgtg gggcctactg |
| 12181 | tgttagcctc actattcaaa ctagatRtct gggtatcctt tggagaacat ttaaggtaga |
| 12241 | attgcctatg atgtcagctc tactgaaaca tgattttatt atatgtaaca ttatgaaatg |
| 12301 | actaaaatct ccaggtggag tttaagtaat cattcccatt gattctcaag ctgggatatt |
| 12361 | cttcaaaaaa tcattcccag gctgggtagt ttatattatt gatataatta tgacaccttg |
| 12421 | aaaattcagg agagcattaa ataatgtaat gcttgccgca aaccccagtg ttccttacac |
| 12481 | tgcagagctg cactctgatt agagtaccat acgcatgagt tttggaatct acgaccaggg |
| 12541 | ttcaaaccca gctctgccac ctcatagctt tatgaaccta aattatgtaa tatatataag |
| 12601 | cctaaatttg tttttttctg gaagtagagt taatagttcc tcccttatca tatttttatg |
| 12661 | aggaatgtag tacaataggt gaagggatta acatagtgcc tggaacatag taagtgctca |
| 12721 | ctaaatggaa actatcagcc ctcaaattat ttctgttgaa agagtgtttc agcttctaag |
| 12781 | gaacgtagca aggttctgga ctcagggata tgaccagcat taaaagtatc atgatcacca |
| 12841 | gcacaggata taattatatc ttggtctcag agactcttaa gtttatccgt gcccagataa |
| 12901 | tctgccaggg attgatggca aggaaagtaa cccaaaccac tctttctttc tgagataata |
| 12961 | caattacatc catgatctga aaatcatgtc taaacaacta cgtgtaactg tccttcatca |
| 13021 | actctatcca tgtctacacY aaagatgatg ttgggtgtta aaggtattgg ataaaataca |
| 13081 | atgaatgttt ctgagggcta tcatgaataa actgagggct gtggactagg tcctctgggg |
| 13141 | tacataaact tacagttatt gccatgttat atgatgtgct gtggaagtaa ctaaagagac |
| 13201 | cataaaggtg aagctcagga gagcactgca gagccgtgac tttggtgtga gtcctaaaga |
| 13261 | ccagtaggaa gtcatcagac agagctgcag ttttggtaag gggccttcct agcccatgga |
| 13321 | acagcatggc aaatgtgcct tcagggtaga ccctagggag tcttttcaat aaaataaaca |
| 13381 | tgacctacca agttcatgcc atcaagatgg cattgtgcct aacaccttag cagcacgtca |
| 13441 | tgagacctga aaacacactt agtacccacg cggcttctac tctcttattc aRagaaagca |
| 13501 | tccagagctg gaaacatttt gccttgtttg aactcacaaa ctacaggaaa ggagtgctgg |
| 13561 | aactgtggct cccaaaggtc tgtttcatta ttctctttat agtcttctta actgcacaag |
| 13621 | tggggaatga gttgaaagtt agaactgccc agcttaaggg cctcttgctc tgaagtgact |
| 13681 | tggagaacta acagttcccc aggcagctaa aatctactga acctactgta gatctttcag |
| 13741 | taaaaagttt agctgatgaa aagtgtggtt tttaaagcac catttgtggc tcaggtccag |
| 13801 | gKttgaatga tactccttat ttatgaactt ctgtatagct gctgtctgta tggcctcttg |
| 13861 | aaggataagc cacagtgagg ccagcctttt cttaaatagg gaatgagggc aacaggaSaa |
| 13921 | cagtgaattg ttagggggca aaccaaattg gcatcatttt ctttgagcaa aactgtctct |
| 13981 | tacttctggg ggaggccccg agcatcccac ctgttcttgc gaatagagga tgtccatccc |
| 14041 | aactctctct tctccacaca cccattccct gttccctacc ccctcatgat tcactagatt |
| 14101 | aattatgcac cacactgaag tgatccccag ggtcatgcca tcagaccctc atYgctggtt |
| 14161 | acctgttgcc aaaatagtcc aagttcccat gccatttttc cttcatctat ttgcttatcc |
| 14221 | ccatattgcc cagtgtattc tctcatttgc ttctatctcc aatggcctcc ccctgcctag |
| 14281 | gacttttcca ttatcccctc tagatcttac aatgccatca ccctccccta tgtcctcttc |
| 14341 | tctgaatcat ggttgcctcc tgaagacacM atcttccctg cagccctctc aagcctagct |
| 14401 | atcttcctct ctacaacctg cgtatcattg gcctgaaggg gagacatctt gctttgctat |
| 14461 | ttgcagctac tttcagacct ttaatccttc ttcctctcta atgaccttct tacaagcaca |
| 14521 | tgtctcctta tcattgactg aatattctag cacctaaccc actcaccctc cccactcata |
| 14581 | ctccattcac catcttggcc actcagctcc ttgacgactt ttgaaaatct tgttctgccg |
| 14641 | ctgcctgaac atcagccacc acactcatgg ttacactgcg tactgtgtca tgactaatca |
| 14701 | ctggcatcgc caacagttcc atttcaagca tcccacgctg tggtcatcac ttcttctctc |
| 14761 | tctagtcact ccccttagca ccatcactcc aaattggtaa gttttcagcg actatcatcc |
| 14821 | cccatttcat tacttccctc cttgaccagc ttagcttcta tggttccttc tcataattat |
| 14881 | tctcttgccc ccatttccaa atcccattat gtactctccc agcactgtac tactcttttg |
| 14941 | agaattaaaa agaaacaaat tgcaactcta gtaatttttt cttactctta acctgtgcct |
| 15001 | aaaaaaacta aacatgactg gagaacaaaa gaccaccaag ttgacctgtg ttcctttgta |
| 15061 | tttatatKaa tatggctcac atggttttca tcactgtcca gtaacttatt gtcacagttt |
| 15121 | ccaaggtcaa tttcacactt ctcaaacttc cacaaactct tcattgtcta ctgacaacct |
| 15181 | tacttctatc tttactgaga caacaaaaaa aaaaatcaga tgagtatgta cttatgccca |
| 15241 | tcacgtcacc gtattcccaa actacctgcc tctgtgcaca ccacctcact gcctgctgca |
| 15301 | gttactgtcc agggccagcg cctctgccca tgtactggag cctgtccctc cacccttttc |
| 15361 | aagcatgtta ctctatcaaa taaatatccc tttctctttt gcattatcag ttttgctatc |
| 15421 | tctctgttgg ccccaccagc actattaccc atgctatatt agcttttaaa aaaWtctctc |
| 15481 | aatctcacat ttatctccaa cgtttacatc attcttttgc tgcactttgt agaaaaatat |
| 15541 | tttgaatttt ctgtatctat ttctacttcc ttacttccca tgttttcttg aactcactcg |
| 15601 | agttactctt ttcttgctca atactggata gaaactgctc tcattggggt catcgatgac |
| 15661 | cttcaaacag atttagtcca actgtgaatc ttcagttccc attttactca gcctctcaat |
| 15721 | aacaatccgc ccctgcccat tctctcactt ttgaaacacc ttctttactt ggcttccagg |
| 15781 | acagcacact cctggttttc cctctacctc actgaacatt ctggaatacc ctcctgtttt |
| 15841 | ctgaggtctg tatgttggag tgagaggcat ctccatcttt ggttttctct tctttaacta |
| 15901 | cactgtcttt tggtgggctc atacaggctc ttggctttta atactactta taaactgatt |
| 15961 | actctcagat ttctgtttca gctcaccctc ttccttgaac tccagcttat aagacactcc |
| 16021 | tgctactcca cgtggatttt tttttttttt cttttttttt ttgagataga gtctcactct |
| 16081 | gtcacccagg ctggagtgca gtggcttgat atcggctcac tgcaaccccc gcctctcggg |
| 16141 | ttcaaacaat tctcctgcct cagcctgagt agctgggact acaggcacat gccaccacac |
| 16201 | ccagataatt ttttgtattt ttagtaaaga tggggtttcc ccatgttagc caggatagtc |
| 16261 | tcgatctcct gacctcatga tctgcctgcc ttggcctccc aaagttctgg gattacaggc |
| 16321 | gtgagccacc gtgcccggcc tccacttgga tatctaataa acatctccaa ctgatattcc |
| 16381 | aaaaacaaac atgattttct ccccaatgtt tttctaaccc attgttcatc atctcagtca |
| 16441 | actgtaccat catttacttc atggtttagg taaaagaatc tagagccatt tctgatctct |
| 16501 | tgcttttata tcacaatatc aatctatcag caaatcttct cagctctgcc ttcacaatag |
| 16561 | gtccagaatt caaatccttc tcaccatctc cttttcctag tctaagcccc atcatgtctt |
| 16621 | tctggattaa tgttttatcc tacgatggat ttttttgtcc ccattcttat ctttcttcct |
| 16681 | aagagtcctc tttactcagt agccaaagga atccttttaa aaagaaaatt tagcttatat |
| 16741 | catttccttt ttcaaatcta accaccatct tcccatattt agaatcaaat ctcaagatct |
| 16801 | tgccatgctc tgacccctgc ttctctctcc aatttcattt acaatcccat ttactcttgt |
| 16861 | tcattctgct ttatccacaa gactctttgc ccctccttgt ctatataaac aagctctccc |
| 16921 | tccaaagcct gcccctgctt tctcttcaga gtctgaaatg cccttctccg tgtcaatagc |
| 16981 | tcactctcac ttcagtcagg tctctgagca cttatcccct cttctgagag gcttgccctg |
| 17041 | accaccttgt ctaaaatagc atccctttcc caccttactt tttaacctgc tttgtggtct |
| 17101 | tcgtacttac ctctatStga cttctccact agaatgcaga ccatgtgaag gcagatttgc |
| 17161 | tgacttcatc acatcaacta tgtgtaccta gaacagtcct tggatggttg acgtgcttga |
| 17221 | taaataaata tttgttgcat gaatgaatga gtgaatattt agagctctat gtcaaatttc |
| 17281 | tggttcagat gttcagggat gttctcagtt ggacctttta ataaaaaaaa acaataaatg |
| 17341 | accaaattct actttaagtt attttagggt tccaacagtc acccatagac tgaacaaact |
| 17401 | ccgaagattt ggcttcatgt aagaatgtca ctgcaaagaa gcttcagtac ccattgtgat |
| 17461 | atcatttgct aatttggaga ttgtgatgcc aaaagtataa gccccatgct cagtcagcat |
| 17521 | ctaggtccaa tagctctcag aggaaccata catcaaagca aactttctgc aatgcggaaa |
| 17581 | agaacatcca tgcattttta aaatgaattt taaattttaa ataattttta aaagtggttt |
| 17641 | catctcttgt aatggaaaat attcaaaatt ctcatgctga ttcatgaagt atacttatat |
| 17701 | aattagacaa aataagaaca ttgaaagaag cagactcctt gtttcatcgc tttaagcaaa |
| 17761 | taataaaaat ggactcRtgc aagtcacacc actaatttaa atacattttg taaaaagata |
| 17821 | gcaaataaat gaaataaaaa tatttgcttt agttttttta aacaaagatt tattaactct |
| 17881 | gaatttttta ggttaaggac catgcaatat atatctccta caccgaagtg tccaagaaat |
| 17941 | gtttttggag taaattaatg aactgcttta gaatgttgct cagaaattaa gctagaaatt |
| 18001 | cagagcaata ttctcagggc ttgatttaca ccaaatccaa gttcgcaatg tattaacatg |
| 18061 | aaggaatagg ccattgcttt ctgaaaacct tccttttata aaagaaatac tttaatatta |
| 18121 | tgaattctct tggtctgtgt aaaatattat ttgcattata aaaattgatt ttttggtata |
| 18181 | attgttcata ggcaatattt tcaccaggat gggttcacaa gtgttaatgg aaggcgaatt |
| 18241 | atgtgtggga cagtcttcta gggaatatgt gaggggtttt taagatggat aacattttgt |
| 18301 | acctgctttt gagttgcaaa tgatctggac tgttggttaa acatcgcaga caagatcagg |
| 18361 | tgaagaacat tactagaagt tcagtgatta agaaataaag ccacgtaccc cacggagtga |
| 18421 | aatttgaatt ccttaaattc cctcaaatta ttttatgttg gattttacac caggatagga |
| 18481 | atgtctttaa aagtaagaaa tatttgacat aaggtagaaa atttcaaagg agtgtttgac |
| 18541 | ttgttgctgg atgtgtcacc acatttcatg tgtctgagaa atataaattt ctacagcaca |
| 18601 | tttttagtgc cttaaacaga ctacaatgat gtaaatggtt gaacagttga aatttagtct |
| 18661 | ggaactggga cagatgactt gccacataga gtggctccca acataaagtg atctgtgatg |
| 18721 | gcaggacttt cagcgtgttt cactattagc atgtgtcatc caagcatgcc tttcaacatc |
| 18781 | tgtggagaaa agacatgtta caaatacaaa acaccaccat gatgaattgg gaatgtcaaa |
| 18841 | aacctctgcc ttggctgtga tccagtgttg ttattggcaa gtagattcat ccttctaggt |
| 18901 | tggttgctcc ataagtaatc ttcaagaaaa gcaagtccaa cacccttttt tctccttact |
| 18961 | gtgaacttgg tcaaaattac caaatgtttg gtaaccattt atggtatcag gaatgcacca |
| 19021 | ataactcctt ctgactcttt aagagcaatc atcttgtgga atgcaatttc ccttcttatt |
| 19081 | attgtgggta tttttaggta cctatgcttg acctttttta aagagagtaa ggatctgtga |
| 19141 | tttaaactag gatagaagct gttccagaga taacaaaaag ctgttgcttt ctatattcca |
| 19201 | acaggaacta ggtaaaacga taaatgtgat aactcaaaat aatgtgtatg tttatcttcc |
| 19261 | aggctttcta atagttaatt ggttaaataa aacattattt acagaaatag aagagccact |
| 19321 | cctcaactgt tcaaacactg caaagtaaac taatgcaagt gcagtgacct cttcatccag |
| 19381 | ccacaaaatc atatatattt ttaattctcc aaggagtttg agatagtaaa gttggatatc |
| 19441 | atgtagctaa acaaaggctt ttaccttttc tcttggaaaa tagttaaaat gaatagRaat |
| 19501 | ctgataatta atctgggaaa tttaaccctg ctctacttac ctaatgcccc cccaaaagat |
| 19561 | gatctttttt ttcttcattg attttatacc ttaaaaagaa attactgaat tgcagatata |
| 19621 | caactagtat ctacagacag aaatgMaggt cagttttaga agaaaccagt gtcaaatgaa |
| 19681 | aacacagatt tctacatggt cacattcata tcacacactc ttgacccaag gtgagaaagt |
| 19741 | aggtcattcc ttatttctta tttattccct ggcacaatca gaccacacta caagtttctt |
| 19801 | ttaccagttg ccaaaatgaa atcatgacac ccttcagagc tactgtcgtt acatgcaact |
| 19861 | caagacaacg agcagcatca taaccattgt caacaggcaa actccaacaa tgtctgggtt |
| 19921 | tggatttggt tttatgttga gagaagcaga tttcctctga ttttaataat ccataagaaa |
| 19981 | cagaaatcat aatatattga ctccatatta cctaaaaaga gtcaaaagaa gcagaagttc |
| 20041 | tcccccgatt catcctcttg ttccacagac attttgaaaa taaaagaaga caaaatagaa |
| 20101 | aggaaagggt gtgagaaaga acacagaaat gacttgaacg atttcaaatc cttattaagc |
| 20161 | atcactgagg ttgcatttta cccattagac tgactgaatt tagttctcac tgctcagacc |
| 20221 | ttgcaaagaa actggacgta catggttttg ccttccaggg ttgatctaga actgtaaggg |
| 20281 | tgcatgtgaa tgtgggtatg catgtgtgtg tgctttaagt ccattttata aacactgaca |
| 20341 | caagacaatt ctcatcattg gtaatagtct ttggagtcca atcatgcaga aaaacaggca |
| 20401 | ggtgcaagtt aggtctcatt gcaagatgga catggcaagg tgaacagata atcctacata |
| 20461 | aggattggga gatttgtgtc cattagaata tactggccat tggtcccaca taagtaaagt |
| 20521 | tacatataca agattctata tataaacaga ggcccctcac ttcagtttgg ctgaccaaag |
| 20581 | aaacaggtca aagtgagcca ttttcagata gttaaaggac taagtaccaa aagaaataac |
| 20641 | aaaatcttat cctctaatca caaagaaaat agctaaaata actatttaca ttgcaacttt |
| 20701 | tttgttgttt ttacagagag gagtttcctt gcttggctta atgatatctg tggaaaatgt |
| 20761 | tgaactctta taattaaaat gtgtgtgggg gattgtgagt ggggagttgg gttcctcttt |
| 20821 | ggatgcaaag tggctgagat caaaaatttc tagagcaatt ttactcctct ttctgagctg |
| 20881 | tagaagaata catttttcag caaagctggc taataacatg ccttacagag cataatgcac |
| 20941 | ttaagatgtg gcttttcata tgttaagaga ttcaaatcga aatagatttt gtataattta |
| 21001 | ggttttgcaa tgaatattta caattttaaa acaaatcaag tttgcttatt tttttttcct |
| 21061 | tgacaggaca taaaaggttt tacatatttt tatctatttt ttactagttc catttactaa |
| 21121 | ggccaacttt cagttttacc agtcagctgc tatcagtgtc tttagataat atttttctga |
| 21181 | taacagattg aaaaaactat cgagcttctt aaccctcaat taaatagctt actccaattg |
| 21241 | gtgggaactg gccaggctct ggtccaactg ttgggtacat tcctacaaaa ctcagtcatc |
| 21301 | agattcttga agatgaaagg ttattatcag tttttattcc acaagaagaa attccgttgt |
| 21361 | aaggctagtg tattcctatt taaaattgaa cacgctgacc cacaaaacac atataaaaat |
| 21421 | agtaaagaca tattaatagt aaagtggtgg cggttttgcc aaactcttgc agcagtttaa |
| 21481 | ttttccctgc acatgagtac acagccatcc ttcctacaaa atactgcaaa ctattttatt |
| 21541 | aaggatcact gcaaaccaac tgccccaaag gaagcctaac agaaggttag gctcctcaga |
| 21601 | gtgccaactt catgttccaa aatgagcccc aaccccaggt agaaccttca ctttttttca |
| 21661 | ggaatattgt ccaggtgact tgacacttgc ctaccggaaa agttgggatg ttcttgggaa |
| 21721 | ataacaggtg actatttaag aaatatttgg Kgtgtgtgtg tgtgtgtgtg tgtgtgtgtg |
| 21781 | tgtgtgtgtg tgttaagaca gacaagtgaa tgcagaacat attatattct gtgaatttga |
| 21841 | taatatcatc acctcctaga catattatag gaaagcagac ctagtgggca aaggactggg |
| 21901 | gggcttttac agtggtgatt ctctttgttg ttccatcatc tgccttgctt taatcaccta |
| 21961 | ataaaactga tcctgcaaaa attctggggg ccaaatcctt acctctattt gtaaatgata |
| 22021 | cttatattca aggaatttgt tccatttttc tcttgtccat aagggaaatc tggctaaagc |
| 22081 | taaccagctc ccaagagcca tgcacagaag atgtaagact aattgagctt tgggaaggta |
| 22141 | ctaacagcgg gggaggggcc ctgtctagtt cttgcctggt ttacYctgag ggggaaattc |
| 22201 | accacttatg caggtacgag agatagcctg tcaagcactt tgaagatgct ggctggagac |
| 22261 | ttccttctgt cttgtgatgt catcttgcgg ctgatgaaat tccccttgag actctcttta |
| 22321 | aagcagcttc ctgctctccg cacttaatcc catgtggtat aaagtaaggg aggggagaag |
| 22381 | ccacagatat atcaagagaa aagtaccgag aaagaaaatc agtaaagaat tagagataca |
| 22441 | gagaggagaa gtttttaaaa aaaaaaaaaa aaaacaggaa tatgaaagag agggagctca |
| 22501 | agtgactgag gaggcatttt taacaatcag gttatctgca atattaacca gaaaaaaaat |
| 22561 | ggtagcaaaa tagagaaaat gggccatttt tattccaggg gacaagctgc acaaaggaat |
| 22621 | gttcttctat ttattttaaa caaatgactg cgtgtactga atctgactgt gtgaaataat |
| 22681 | ctcagaatgg cagcaccact ggYatggcga tggtgcaggt gggtgcagtt ccctgtggtc |
| 22741 | tctattgctt gaagagagaa agRaagttcc ctattattat atttaaggca gttttcagag |
| 22801 | cactggcatt cttgtttgct ctgttttggg gatattctat ttgccaatca gttcacactc |
| 22861 | atcaaccatc cacttcctgt ctttcacttc ttcaggtgaa atatcaccac tctagtgagc |
| 22921 | accaacatgg tcagagtgca aataactgtg atggatactt cgaagtgaat aagaagggaa |
| 22981 | gcctgattat tgctgagatc accttggtat tgccttgtac ataaccaaga tgtttcacag |
| 23041 | tgcctcctta agtgctcagg aaagcacttt catttaattt aatttaagct aaattctgga |
| 23101 | gattttgcta aacattgaca ttaatgcttc atggctttta agacaaactc atattgctaa |
| 23161 | aactatttgg cagcagtaaa tttatgggca gagttattgc tcttacatct actgcagtac |
| 23221 | aaaactcaga tgatcagccc ttcgattgct catacttatt aaaataaata aataaacatg |
| 23281 | aaacatcagc cacattaagg agtcagggaa actcctgagg aataacaatg tctctggaga |
| 23341 | tgttttcaaa tgctaacctg agactcacac tgtggtttgg gggtttcatt Mttattatta |
| 23401 | ttattttatt tcagaagagg aaagtaatat tatttctcaa aataaaactt tggcagagta |
| 23461 | tgaatcaggg aagcagccca cacatggaaa acacaatcca caatctgaaa gtagtctagg |
| 23521 | caggctcatt tttttgttga aggaattcac atggaggcaa ttctgctcag ttccagagag |
| 23581 | agattcttat catcaaaagc acacacaata accttgcttc accacctgcc actttctcca |
| 23641 | gtggcctctt gttgtgacca gtaattgaga aatgacaaat gggtatagag ccaaatctct |
| 23701 | aaaacaggga caccagtgtc tcctagtatc tgggtaataa cctcctttca gtcttagtcc |
| 23761 | acaccacaca ctaccctcca gtctctccta ctctttactg tcacagaaaa gactctcagc |
| 23821 | ttttattcac acctgccttc Wgaaggccaa accattttgt gcctgaaaac tgttacccac |
| 23881 | tcSccccaca ctttacataa gtatcacata tgttctttct acaaattggt atacaggtgc |
| 23941 | catttaatcc attcaaattt ggaagctaca tcttcaaggg tctgagagag ctcactcccc |
| 24001 | ccatatattc cccctttaca tgttttctta taagacatac agtttaatca attaacaaac |
| 24061 | taaacagctt atatactggc aatatattac agatgggttt atgtcagagt aatagatcac |
| 24121 | atgaaatgga cYatgtggta ccccagtgca ttatgtcttg gtagagccYt gaggacactg |
| 24181 | acagtagcat ctctaagtaa gtagtgctgt atgaatacag acacatgcgg atctgtatct |
| 24241 | acatccatct gactaggcca aggagcaggt agatgcaaga tagagacaca cacatgctgg |
| 24301 | atggggccac tgcacacctt gtcatgccat ttaaaagggc agttacaggt tgccggtttt |
| 24361 | gcagccatta aaattacact ttatggaaaa ggcttctcta attgtgtctg atttgctttc |
| 24421 | tgtagtaagc atcattggaa gaagaccaaa gcaagagcaa ctagaagtta aatatacatt |
| 24481 | tgagcttagc tgcaatacag aaccctgggg aagaagcagg ctgaaagatt tatttaattt |
| 24541 | taggcttgga ttctctccct tgctgttttc ttgatgttgt gtgtctgcat ttgtgtgtgt |
| 24601 | gtgtgtgtct gcgtgtgtgt gtgttccatc acatcatctc ctgaagaacg ctgggtttcg |
| 24661 | caggcaggcg gctagtcacc tgcaacaacc caggggtcct gccgaggctt ccaggctgct |
| 24721 | gtcagtgtca gatgccaggg tctggtcggt ggacatggag gagatgtcat tgacagacga |
| 24781 | ggatggaggg agactctcac tgctgttcac tgctgcacct gtgctaagaa aatgaagacc |
| 24841 | aacatgactc aatctagaac cagacccatc ttaatgccat tcaggattca gggatgggca |
| 24901 | aatacaatag gggatgtcca gtccatcaat catttggcaa gcctttatag agcagctgac |
| 24961 | ataggggagc atatcaaatg gtgacaatca agacatagca ataaaaagtc cttcatcaca |
| 25021 | agttatcaaa tctcactcta tatttgaaat cacctgcaac actttctatt aaaaaacact |
| 25081 | aatatctggt cacattgcct cccctgcccc cactgcactg catatatttt aatataattg |
| 25141 | gccttgggtg aggccaaggt atttgcattt ttatttattt attttttttg agatggagtc |
| 25201 | tcgctctgtc gccaggctgg agtgcagcgg cacaatctcg gctcactgca acctctgcct |
| 25261 | cccaggttca agctattctc ctgcctcagc ctcccgagta gctgggagta caggtgcgca |
| 25321 | ccaccacacc cagctaattt ttgtactttt agtagagatg aggtttcaac atgttggcca |
| 25381 | gaatggtctc gatctcttga cctcatgatc cgcccacctc tgcctcccaa agtgctggga |
| 25441 | ttacaggcgt gagccaccac gccccgcggg catttgcatt ttttaaaagc tcctcagaga |
| 25501 | ctgtattcag ccaagcattg aaagcctctg gcttatgcag gagacaaata agcaaaaccc |
| 25561 | cttaatggag ccatctgagc ctggttgttt ttaatgcagg gagcttttaa aacaaacata |
| 25621 | tgcaagggtc ctatcccaga aatagaagct gaagtgaagt tcagaaatct gaattacttg |
| 25681 | gggaccacat ctctagagaa aggaagatat ataaatacta ttatgtcatc attaatgaca |
| 25741 | ccaaacttaa aatgcatata gaaaagccag taggacataa gtaggactgt tgatgaaagt |
| 25801 | tacaaaaaaa aaaaaaatag gagaagcctt agcacagctt ttgcttatga gctaactctc |
| 25861 | cagcaggctt gcttacctgg gatctccaaa taagaactcc aaattattaa cttagattaa |
| 25921 | cttgaaaaaa ttgtttatct gttatgcggg agtataaggt tgaaaccctg agaagaaacc |
| 25981 | tgcccaYggt aatcactcag taaaggctag ctgagttgcc agtagctttg agatcaacaa |
| 26041 | aagaaagatg aaccaaaggc attgtgcgat aYaattaaac cagagcaccc tggaaacaat |
| 26101 | ccatttactt tttttctttg tatattcttt atacctaagc aatctgagtg gtaacacact |
| 26161 | tgctttgtga aatagctcgc tattaaaaat ctgtagtata gtcccctaat caaataagag |
| 26221 | atgaggaatt ctcactaaaa ttatatctta tttgagcaaa tatagtgtgc aagatctatt |
| 26281 | tggtgaaaaa taataactcc aaagtcagtt ataatagtgt tatcacatag acctaataaa |
| 26341 | tcagaaagac aaaataaaac taaattaagg aagggRagtg gttaacataa tccttgctcc |
| 26401 | tggagtagac atttcttgcc tgtgagaaaa gtgtgatgaa aatgtatcag ttatctagag |
| 26461 | aggacacaaa cacctaaact aagacagatc acctgtatct tccatgtatt tttaagttgt |
| 26521 | atttgttgac tataaatata tggacgtact ctatgggaaa tgtgaaaaaa tataggaaat |
| 26581 | aaagataatg atatgcaccg atcttcaaat aaaYggtaag aactgttaaa cttttcatat |
| 26641 | atttcttccc ggtcattctt tttgcctgga agtttccctg gtcgtgatct tttaatatgt |
| 26701 | atttttttgg gatcttgata tttttaKata cagaaaagct tatgaatttt tcatgttctt |
| 26761 | taaactccta aaaataccca tcctttctaa tcttaaatgt ctctagaaaa ggtttttaaa |
| 26821 | gtcctcYtgt tattgtcttt ctgtaaataa ttactctatc aggacaatgt ataaagttat |
| 26881 | aatattccat aatattccaa ctttgaatgg ctttaattta ttccaacttt acttaattat |
| 26941 | atcccccata gctatggtac agagctggtt catttcccct catgataaga ttttatctcc |
| 27001 | tagcagacag cccttggttc ctttacattg cccaaatgcc ctgctctgtt tttcaccccc |
| 27061 | gattctggat taaattatac caaYatcctt catattcttt cataagttgc taataattta |
| 27121 | gtgtcactat gatggactcc tcagaattct cttccattta tccacactct tattaaaaga |
| 27181 | cagagattaa aactggacac aggactctat tataaaggcc tgatcaattg tgagcctagc |
| 27241 | agagggaaat tgcttttttc ttactatttg tctgctggat gacatatccc tagactatgg |
| 27301 | atgcctttca aattatggtg ctacagctta atttagtttt gacttgaggt atagaatgat |
| 27361 | gctcatatat tttttgtaac agctttatta agatataatc cacatatata attaagaaat |
| 27421 | ttaattttca tgattcagtg ttttttagta tattcacaac attgtgcagc catcaccaca |
| 27481 | atccattttg cagcatttca tcatccctaa agaaaaccct gcactccatt cctcacctac |
| 27541 | cctgtagctc taggcaatga ctcatctact ttttgcctct ataaaattgc ctgttctgaa |
| 27601 | catttcatgt aaatggaagc atatcatatg tgatcattta tgactggcca ctttcatata |
| 27661 | gcagaatgtt tttaaggctt atccatgctg tggcatgtat ctgtatttca tatctttgta |
| 27721 | tggctgcata atattccatt gtatggatac atcacatttt gtttatttat tagttgatag |
| 27781 | gcatttgatg gatatttagg ttgtttcaac tttttggcta ttacaaataa tgctgttatg |
| 27841 | aacattcatg cacgagtttt gtatggacat gtgttcattt ctcttgggta tacacctaga |
| 27901 | agtggaattg ctggggcata gagtaattct agctttaaca cttgaggaac tgacaaactg |
| 27961 | tgtccggaat tggtgggttc ttggtctcac tgacttcaag attgaagccg cggaccctcg |
| 28021 | cggtgagtgt tacagctctt aaggtggcgc gtctggagtc tgtcccttct gatgttcaga |
| 28081 | tgtgttcgga gtttcttcct tctggtgggt tcgtggtctc gctggctcag gagtgaagct |
| 28141 | gcagaccttc acggtgagtg ttacagctct taaggcagcg agtctggagc ttttcgttcc |
| 28201 | tcccagtggg ctcgtggtct cactggggtc aggagtgaag ctgcagacct tcgtggtgag |
| 28261 | tgttacagct cataaaagca gcatggaccc agagtgagca gtagcaacat ctactgcaaa |
| 28321 | gagcaaagaa caaagcttcc acactgtgga aggggaccca agtgggttgc caatgctggc |
| 28381 | tcgggcagcc tgcttttatt ctcttatctg gccccaccca catcctgctg attggtagag |
| 28441 | ccgagtggcc tgttttgaca gggtgctgac tggtgcattt acaatccctg agctagatac |
| 28501 | aaaggttctc cacgtcccca tcagattaga tacagagtat caacacaaag gttctccaag |
| 28561 | gccccaccag agcagctaga tacagagtgt ggattggtgc actcacaaac cttgagctaa |
| 28621 | acacagggtg ctgattggtg tgtttacaaa ccttgagcta gatacacagt gccgattggt |
| 28681 | atatttacaa tccctgagct agacataaag gttttccaag gccccaccag agcagctaga |
| 28741 | tacagagtgt cgattggtgc actcacaaac cctgagctag acacagagtg ctgattggta |
| 28801 | tgtttacaat ccctgagcta gacataaagg ttctccaagg ccccaccaga gcagctaaat |
| 28861 | acagagtgtc gattggtgca ctcacaaacc ttgagctaaa cacagggtgc tgactggtgt |
| 28921 | atttacaatc cctgagctag acataaagac tctccacgtc ctcaccagac tcaggagccc |
| 28981 | agctggcttc acctagtgga tcccgtacca gggctgcagg tggagctgcc tgccagtcct |
| 29041 | gcgctgtgcg ctcgcattcc tcagcccttg ggtggtcgct gggactgggc gccatggagc |
| 29101 | agggggtggt gctcgtcggg gaggctcggg ctgcacagga gcccatggag tgggtgggag |
| 29161 | gctcaggcat ggcgggctgc aggtcctgag ccttgccccg caggaaggca gctaaggccc |
| 29221 | ggcgagaaat cgagcacagc gccggtgggc cggcactgct gggggaccca gtacaccttc |
| 29281 | cgcagccact ggcccgggtg ctaagtccct cactgcccgg ggccagcagg gctggctggc |
| 29341 | tgtcccgagt gcgggggccc gccaagccca cgcctacctg caactccagc tggcccgcaa |
| 29401 | gcgccgcacg cggcccgggt tcccgctcgt gcctctccct ccacacctcc ctgcaagctg |
| 29461 | agggagtggg ctccagcctt ggccagccca gaaaggggct cccacagtgc agtggggggc |
| 29521 | tgaaaggctc ctcaaatgcc gccaaagtag gagcccaggc aggggaggtg ccgagagcaa |
| 29581 | gcgagggctc tgaggactgc cagcacgctc tcacctctca aaactgtttt ctaaagtggg |
| 29641 | tgcaccattt tacattacta ccagcagtat atgaggattc caatttctct acatcctcac |
| 29701 | caacacttat tatctgtctt tttatctagc catcctagtg tgcatgaaat ggtatctcat |
| 29761 | tgtggtttga tttgcatttg cctgatagct aataatgttg agtatatttt ttcatgtgct |
| 29821 | tgtcagccat tagtatatct tctttggaga aagtttactc agctcctttg gccatttttt |
| 29881 | aattgcttta ttttcttttt attattgagt tgtaagctct ttagctcttt cttcctttct |
| 29941 | ttcttgcttt ctttcttctt tatttctttt tttaaggaca gggtctcagt cgcccagcct |
| 30001 | gtagcacagt ggcatagtca tgtttcactg tatcctctaa ctccagggct caggcaattc |
| 30061 | tcctgcccca gcctcccaag tagctaggac tacaggcaca taccaccaca cctggctaat |
| 30121 | taaaaaaaag attttttgta gagacaggag cttgccatgt tgcccaagct ggtctcaaac |
| 30181 | tcctggcctc aagtgatctt ccgatttctg cctcccaaaa tgctgggtct acaggcatga |
| 30241 | gccactgtga caggcctgta agagctcttt atataatctg ggtacaagtt acttaatgag |
| 30301 | atatgtattt tcaaatgttt ttcctctatg ctatggttta tcttttcatt ttcttgaagg |
| 30361 | tgtcttttga agcacacata tttttaattt ttatgaaata gcatttatct gttgtttctt |
| 30421 | ttatcacttg tgttttagtg tcatatctaa taaaccattt tctaatccaa gattataaaa |
| 30481 | acttactcct atgtttttgc ctaagagttt tataatgtta gaccttatac ttaagtctat |
| 30541 | gattcatttt tagttcattt ttgtatatga tatgaggcag gggcccacct tcattctttt |
| 30601 | gcatgtatat atccaatcat cccagcacca tatattgaaa aggctattct ttgtccatgt |
| 30661 | cttacatcct tatcagaaat caattgatca taaatgtagg tatttattcc tggactctca |
| 30721 | atttgattcc gttgatctat ttgtgtatct tcatgcaaat agcactctgt cttgaatatt |
| 30781 | gtagctttgt agtaagtttt gaaatcagaa catgtaaatt tcccagctgt gttgtttttc |
| 30841 | aaggttattt tggatattct gtatcctttg catttccaca caaattttaa gatggggctt |
| 30901 | gtcaatttct gcaaaaaaga cagctatgat tttgataagt attggcttaa tctgtacatc |
| 30961 | aattYgggga gtatctgtca tttcttgaca atattaaata ttctaatcca tgagcaatga |
| 31021 | aatgtttttc cagttattag atcctcttta atttctttca acaatgtttc gtagttttca |
| 31081 | gagtacaaaa cttgtacttc ttttgctaac tttattccta aatatgtatt tcttgatgct |
| 31141 | attctaaatg gaattgtttt atgttcattg ctaatgtgta gaaatacaat tatactgagg |
| 31201 | acttatttga ataagaagtt tcagtgtcag tggattggat gttaaaaaca gatcaaatga |
| 31261 | atatatatat atatatatat atatatatat atatatataa aatgaatgtt gtaattaact |
| 31321 | agaaagccag tggaaccagg cgattatgat agcatgcttt atttatgaga aaataaccag |
| 31381 | ttttaaaaat aactggtttt atgagaaaat aaccagtttt aaaactttaa aaagttttaa |
| 31441 | aataaaattg tggcataata caaaccaaaa agtattttta agtctttaat aataatcttg |
| 31501 | gtcttctctt gagataggct ttttccccta tcctggctgg agttgattat gtagacacat |
| 31561 | tcttattgtc acaattaaag ttgagtaatg attcaaataa gggtgagata agtcttagct |
| 31621 | gaaataaggc tcccttatag taaaacaatt tgatgtttac aaataaacac aaacccacaa |
| 31681 | caatggcagc tagagaaatt aaacaaagag cattgaagaa atttctccaa aacacaaatt |
| 31741 | tatatctaca aataatattg aatgtatgta acaaaagcca aagtagcttt taataagctc |
| 31801 | ttcataaaca ggatcgtgct gcaggctatg cttataaaac acacagcact cctactgcac |
| 31861 | ttattttcaa aatcagcata ttttcagcac acttctggaa tattccgagt gttatgccat |
| 31921 | accagtcagc cccttattat atgtagtttt gttatggatt acctcaagca tatatttccc |
| 31981 | ctgcttcttt ttactgcttt cattatactt catttctcct gtcttccaca atgtctactg |
| 32041 | ctgagtagta gcacagagta agtattcgag ttaacaaaac agtattgagt atcttatgtg |
| 32101 | tactcttcta gataggttct gtgaggaaaa gcaagttcct tcctgctttc caactagtga |
| 32161 | aactgttaag ctatttttgg ccaaaggaaa tcactttgtt gcagttgtct ttcttctcct |
| 32221 | tttgttcatc agcagcttct ctctaattcc tatcctctgt gttggaatgg tcctgccctc |
| 32281 | tagtttatca atgttgaatg caaatataat tagtgttcaa ctactcaaag ttcaaaatct |
| 32341 | atcaaagtct ttatcagctg tatttctagg gatccataga ctattgcttc tgattttata |
| 32401 | tggcacaaat tcattaagag aagtcttcag tgcttYcttt gtaaaagttc ccatgacatc |
| 32461 | tgcttgtttc atgggaatgg ttccttgcta tccagtgtga tggtgggaga gaaccgtgca |
| 32521 | ctgtctatgg tgtcctggag ctggctttta gtggcttctt agaacgaatt attagcatct |
| 32581 | ctttccaact ctgcatccat tgatgttaca ttgttgctta aaattggtca cagtgagaat |
| 32641 | atttatacca caggaattgt taagtgctac tagtcaattc tttttttaaa tatagttgtt |
| 32701 | aaatgttaac cagtccacca ctattagatg aaaactattt gcttctgtat gactccctct |
| 32761 | attgcgctat aataaatgcc tccttgcctg cagccacttc tgctggatga tccattcaag |
| 32821 | Wctattttca tgtaggcaat aaacaggagt tctaaaccat aaacccaaac acaaacgtaa |
| 32881 | gggacaagac atgggccttg gctttaagtt gttaaattac tgccaagcag tttgcgaaaa |
| 32941 | tctcttaaac agaattgaac tgaattgaat atgatcatWg tgttcagttg tccagtcaat |
| 33001 | gtgagaaata tctcttcttt ctatattatt taagagaaaa cagtagagat acacattttc |
| 33061 | aaaaatgttt tgccatcaat aaagcattac aaatttttgt ctataaaaat ggcattaaaa |
| 33121 | ggaactgaaa atgtcaatag ggttgcaaat gaggaatatt agctaccttc aactttacct |
| 33181 | aaaaaatcag atttcataga aagaagtaac atatgctaaa gaaaatttct cagctacaag |
| 33241 | ttattcaacc aaaaaacatt gttatggggg aaaatgttgt tctgcttttt ttaaaggcaa |
| 33301 | tgattttcct gataaccccc aatcccaaca acaacaaaaa aatatggtag acacagaatt |
| 33361 | agaaaaagca caagaatgaa aaaatagtgt cttttttatc tacatgatct tgagccacaa |
| 33421 | tttgtctggg cctgagattt tttaataaaa agtaaataaa attaatggac tatattagtg |
| 33481 | accctaagac atattcccat ggtggggaat gtggtcctca ctggggagag aatggttgca |
| 33541 | tattaaaatc acccagaagc tttatctttg aYttagagat tttgaaaaat ctctgcccat |
| 33601 | cctgttgttc agcatcctta tcttagtgag tcactgttag ttttgggagc tttacttctc |
| 33661 | aggtggaaga gggctggtga acaaactcag aattggtgaa tcagatgctc tcctgtattt |
| 33721 | tttctagttc taacgtacta taacaaatgc ttttagaaga aaattaattt caattcatgg |
| 33781 | actgctagtc cctcagcaat tgttacagat caattcagga agcaagcagc tatgccatcc |
| 33841 | ttgaaaataa attactatgc aagtgaaatc agatagcaga agggaataca attatttatt |
| 33901 | tggaaatgta tctgaagttt agatttatct cttattgaca tacaacaatt tcatcaataa |
| 33961 | atttcaaacc tataaattat tctgcaaatg cacatttaac ttcctattta cattttaaaa |
| 34021 | gcatcgcata acatgggttt cgtattagaa gtacttggcc cctgagatac tgcattaact |
| 34081 | tactcccttg ctgcagaatt cccagtattg ccataactga atactaacag agtgtgtgtt |
| 34141 | ataaaaatac cacagctatt ttcaaaataa aatatgctac gtgaacatta atgaaaaaaa |
| 34201 | gctgaatata atgaacaatg caaaaaagct gacttttcaa actttttttt agaattgctt |
| 34261 | tttctttcca gcatagttta ttatacacac aaaaacgcat acacacagtg gtgcgccaat |
| 34321 | aaaaatttaa caattggctc tgcagggggc aacaaaaacg ccctgatttg tagggtctgc |
| 34381 | caatttccat gttgtaaatc cttcgtgcct gatttcacgt tgccaacaca ccaacaataa |
| 34441 | atgcatagtt gggaagagag gtacagagtt ggccgtcatg agctggtaga gtcagcgcca |
| 34501 | gcacatggct gccctagagc ctctccatca tgcacataaa tatgtcatac agaaatgcat |
| 34561 | cccataatgt gtagcataca tatgatttct cacgaaagag aaagtcatac tacatgactt |
| 34621 | ataactaagt catgaatatt tttcctagat ttaatccaca atcattttaa ctataaattg |
| 34681 | gttaagccag tcaactatag tttagggcat atgtattaca tagataattt ttaattgtta |
| 34741 | aggtagttgg aatggttaaa aagtggctta gccttctcac tcaggtagag cctaagttgc |
| 34801 | atattttaga attaatcatt aacacactaa gcaggcctgc aaaacatcca tgaatatata |
| 34861 | tttcccaaaa tgaataaatc aaacacctga aagctgcaaa cctttatgtg aagcatgaaa |
| 34921 | aaatgccaat agcaagaatt tatatacttt atgcttggcc tgtttatctt catggagatt |
| 34981 | aatagagtga ataaatattt ataataatgg tgtaagaaca tgctgaatag tatactgtat |
| 35041 | taaagttaca tgatcttgtc ttgaattttg tgtttggaat agtactaaga aatatgctga |
| 35101 | gggagaaatt gagtggcaat tttcaaatta atatatattt aYtgagctac tactatgtgc |
| 35161 | aaggcacagc acccaggcac tacatgggac acaaagataa atgcatacag tcttctcaat |
| 35221 | gaaattacaa tcaataMggg ataataagac aagaggtcaa atatttaaca tacagggact |
| 35281 | agtaggacct atagaaagga aagatcctat tatttgggaa gatataaaaa gtcttttggg |
| 35341 | ataaggtggt atgaaaagga aatgaagtaa tgataacttg aactgaagat gaatagaata |
| 35401 | ggttctacta aaatttgtcc aggcccagcc taacatgcta ggttacccgt ggctacattc |
| 35461 | tgttatttct gctgcttaat gagtacatga caatatgttc tggtcattat aggaattctt |
| 35521 | aaaacaagga gtcattctgc cttagcaggt ggcatcttga ggcttttgga ttctgtcttc |
| 35581 | aagtttttta atgagcaaca ggccaaacgt ggattagttt gaagcagctt ctgtggactg |
| 35641 | tgtcaatctg tatgcgaggg tgaatgagta gagagggtgg tatggaaaac aactcctgtg |
| 35701 | attcccttaa ccttctttga ggaagggaaa aacttgggat aaaactgagt attttttcaa |
| 35761 | gtctactggt gagaaaagca tgtcataacc gaattgcatc tgtttgggta tcatagtggt |
| 35821 | ttggggtgta aattagttat tcgaagagta gtatcgatat gaataattga ttaattattt |
| 35881 | cattctacat cttttttcct aaaataattt aggaaaacaa caccctaatc agtttttctc |
| 35941 | aaaactaggc atttgtagcc cccatttttg ctagcaggag ttgaatgtag tttttaaagc |
| 36001 | tgaaaataaa ccaWcacatt ttctaaaact ctttatagtg agagcatagg tcttaggaaa |
| 36061 | aaatatatta gcattaataa gtaaattgtc tcaagtcata ctaaagcaca ttactaggat |
| 36121 | cagtaaaaaa tatatatgca caattgtgta ttagattctg tggctagcaa acgaaaaatt |
| 36181 | ttccaagctg accttaaccg gagcccatct tggtagatgt ttcaactatt gtcacatcaa |
| 36241 | ccttgagaag agttcaaaca ctaagaatga atgagggaag aggtagcggc tgaaaggatt |
| 36301 | actgagctcc acattgactt gatggtcaaa agggcattat ggctctgaat tttgatgagg |
| 36361 | cacatttacc ctttagccca tgttaacatt ttcttcagga ttcattacta ttaaaattat |
| 36421 | ttatgaaaaa gtttttgtRc tggatcatta ccatcagaat aatcagaatg aatgccacac |
| 36481 | tgaatatcaa aagaaataaa actaaaatca ttataaggac acaaccatgt gatatttgtc |
| 36541 | catctgctct ttaagcaatg ttatgttatt tcttgcaacc cctacacaaa ggccaagaaa |
| 36601 | ttacacaagt actagtttat tggttattca cggagagtga gtacctgaag gagaaggctg |
| 36661 | tccttttact acaccatttt tagtcttttc ttctgaattc attacttcct tgtagataag |
| 36721 | ttctgtaaga aacagctgtg ttattataga aaacaaattt atccttcatc cacagggaaa |
| 36781 | ttcattactt aatgccaaat aattacgttt tgatggggtt gagtaaatta aaaatcaRtt |
| 36841 | gagattcctt tactgctcta tttctatttc tatattgcct tgtaattaRc agagagcctg |
| 36901 | gcacaaatag gtatctagta aagcagttga agaaatcaat gcatgaaaat ttatttctac |
| 36961 | gtacatgaaa catactattt atttccatat acatagtatt tatttctaca tacatttcta |
| 37021 | cataaataac atttatttct acatgcatga aatatttcta cataaatgaa acaagtaata |
| 37081 | catgttttac ttgttttctg aattctttgg gggaagtact aatacattct agctcaatct |
| 37141 | agcaacaatt tttatggctt tgaacatcca tttttcttta tacttatttc ctatgaaatg |
| 37201 | cacacattct catgacttta aattattacc aatccttaca acttccatct cttactgcat |
| 37261 | cttttttttt ttccagactc atatcttgga cttgtaaatc taaacttgcc aaaaaccaaa |
| 37321 | cacattaaaa aatttagttc atttactctc agactggcat ccatttttgg ctttctgaat |
| 37381 | gctagagtta aattggatag tttcagttct gtttactctg tccctttagc taaaaatcat |
| 37441 | tccgccctac tcattcttgc tttgcaatgg cttataatct gtataatcat catcctttac |
| 37501 | atgtacttct ttctccaatt tctcacaatt tagtccatgt cctcctcaaa tgatggttat |
| 37561 | atcctatcac ccacctattc atgagcccct aaggagtgcc ttcctgtcct tacttggatc |
| 37621 | aaagcaactc tttttcatct atatcaagca tcttttttca cttgatccac tttttgtata |
| 37681 | tgttcatttt tccatattca ccaacatgac cctggaatct ctggaccagt ggaatctctt |
| 37741 | tagtatcttt taaaattttt ttattttatt ttttatttta catatatata tagagagagg |
| 37801 | cagagacttt ctctgtcacc caggcttgag tacaatggca caatcatggc tcaccgcagc |
| 37861 | ctcaacttcc tggactcaag tgatcctccc acctcagcct cccaagtagc tgggactaca |
| 37921 | ggtgtgtacc actatgcccg gctaattttt gtatgtttct agcagagaca gggtctccct |
| 37981 | atgttgccca ggttggttct gaactcccgg gctcaagtga tcccccagcc tcagcctccc |
| 38041 | aaactgctgg gattacaggt gtgagtcatc gctcctggcc ctgtttagta tctttataac |
| 38101 | attttctgct tattcctgtc ctttgttttc ctcttagtcc tttatctgat attttcttaa |
| 38161 | tattttttct tttcctaatt tacacttatt aattcctgca aggtagctca aatgttcctt |
| 38221 | cccttcagct cttcccattt tgatctgatc atttgctaca atctgcattt gactttctca |
| 38281 | tatttattct attctgtagt gatttagatt agtagattag attaataggc tactcagata |
| 38341 | cacatatttt ctatgtaaat tctcaagtct tatcaatcca atgagtaaac tcatatattg |
| 38401 | actatctttt attttctaaa gtctaaaacc atctcagaat taattttaat ttcatgcttc |
| 38461 | atatgatgat tttatgtaca aacatagaaa gttgtattct ggaattctga actaaagctt |
| 38521 | gccccccacc atgacaaaca ctgacaatca gtaccatttt aaaaggtatc gacaaacact |
| 38581 | gacaatcagt accattttaa aaggtatcaa tggtaaatgc ctttcattct tgagcaaaYg |
| 38641 | tgacattgct ttcggcMtga aaagacagat gaagttttag gagctttaaa aatggtctaa |
| 38701 | gagaatagca gtcagcaaat cattttttag taaattgttt tacaaggaaa agccttgttg |
| 38761 | atactttctg agttcaataa aataaaaagt atactttttt aagtagtaga cttacctttc |
| 38821 | cattcttcaa ttgtgtgttc tctttcatcc aactgcttgt cataYatctg aggtggaggc |
| 38881 | tgccRaataa aaacaaaaaa taatctttgt gtgataatgt aaacataact aaactcttac |
| 38941 | aaYgcacgag aataccttgc cctcatttat ttcggtgatt tcaaccgtat ataagttatg |
| 39001 | aggaaattac agtagttcag aagttaaaat tttcYtgtaa acttaYggag gattcattca |
| 39061 | tattgcaata tttgctgctg acccaatatt gctgcaatat ttgcagcaga acgcaaatcc |
| 39121 | cgagagctca ctttccgaca tcttctgatt tagaacccac gcctaagcaa ttaatgactg |
| 39181 | gtcttttaaa aaacagacaa aacacttgat tacaatgRcc cagaagctac ttttccttca |
| 39241 | Ytcacttgcc ttataacaga accatgctaa aattcttcta ggctaataag attttgttct |
| 39301 | atttttagac cttccctaaa gaaaagttta tatttctcaa cagaccatag tactagtcct |
| 39361 | caatataaaa aaaattgaga tcaaatcaac ctttttattt ttcatgaaat accatagaat |
| 39421 | gtctttatat attgctgtgt gccctatact taaacgattt taagcttttt ggtttgtttg |
| 39481 | tttatttgtt ttaacctgca gaagctgcca tgactgtaga ggtaggtcac cttgaatccc |
| 39541 | tgaatggccc ttggttagtc ctctctagca gaaatgatgc aatagggcct tcagtctgac |
| 39601 | ttatagagcc agcatttctc ttcccatgtg gagttcaaat gtggaaaagg agggattttc |
| 39661 | acaaacctgc gtgcctactg gaaagtggaa tctcaaataa attctctgac ctcttcttcc |
| 39721 | tgatatttag gctttgggag atctaccatg gcggctaggg tagctccaat tcaggccaag |
| 39781 | tgatatggaa agaaaaaaaa aaggctcttg gaaaaataaa tcaccatgag gtagagaaag |
| 39841 | aggaggctga ggggctacac tggctggcac cggtttaagc gcaaagaaga gctatcaagt |
| 39901 | ttttgctacc ccgtccaata ctccaaaagc aatagttttt gtgaaaaact aaaagagtca |
| 39961 | ctcaataaga gtgatgcaac ccctttagga cacagacctt tcaaaaatgg ctattatagt |
| 40021 | aaggaaagct ctggacaaga ttttcagaga cagcttctgg cttctgtgag agctttttgg |
| 40081 | ctcatgaaca gcaccctcgg gctaMagtga cccttctcta attcatgcag aatctgtggt |
| 40141 | agcttgaatc atgacagaca caaatattca tcctagccct ggcatgctgg ggcctcacat |
| 40201 | cctgcaaagt atctggctat gaaaatagaa gactttgccY ggctgtggtg aagtaactta |
| 40261 | gcataatact gaagtattga gaaataacta ctcacggagc cagacattct tttagcacgt |
| 40321 | ttgactgata ctggggcaga aggctgtaaa cctcacctac tgcatttcag atgtggctgg |
| 40381 | ctgagcagct ctcagcagcc gtcagcactg acaactaagc aaaatgagac tctcacacca |
| 40441 | ccccagacaa tagtaagaac acaacacatt gaacgtatat aaggtgacag gactgggata |
| 40501 | gtcaggcact gctctcagga cctgactgag gtagaacctt ccacctctat aaaggactct |
| 40561 | caaataatag aaacattctg acttagtgag tgaatgagta aaatgaagtt atttttgaaa |
| 40621 | aattgtgaaa ccattaccta aggaagctac cacaaagcca aactctggtt ttgtaaagac |
| 40681 | gctttgccaa gtggtaaatt tccggctttg cctcttcctg agcatgtata atgctgtggt |
| 40741 | taggccgcta tccaagcgac cttgtccagg acccctgcag gacacagtgt acataagctc |
| 40801 | tcagagcccc cagagccctc tgagctgttg gcacctcctt cttcttctaa caatagatcc |
| 40861 | agctcatgat cctgcctggc tggcttctcc atgcagtgga tttttccatt caagttttaa |
| 40921 | aattgaacat ggctcaagag tacagttcct gccaaagttc ataaatgcca ttttcttttt |
| 40981 | gcaacaaagc tattttcagg aggtttccct aggagcctca gtgtacagct gttgctgata |
| 41041 | tataaagcac aaagtacata gacactaatc acaaacaaac tacaagagag aatgcaaagt |
| 41101 | gctgagttca agatttttcc tttcaacaaa ccaagagggc tgctttaaac cacttgattt |
| 41161 | aYagtatttg ttcgacggcc tctgcaaatt tactacatat tcagattttt gattgtttgt |
| 41221 | tttttgaaga ggaaatgcga atgtcacctc cagtgaaagg gcacattcac atacagatgt |
| 41281 | cttctatgtg acaatataaa agaataattg accaataatc cagagagagc aatgttacaa |
| 41341 | cacacacgta taagtgctgg gcagcaatca tcaggatgag atagttcaaa acattccctg |
| 41401 | cctccttccg ctaaggagaa gttggtattt gagcactcgc ttgtgctctt tccctatctt |
| 41461 | atttaatagg aattattctt ttgctaagtg acaatctaaa taaaaaataa atgaggagtg |
| 41521 | gggataaaga aaaatgcccc agaggtgtct gttgagagag tattttagga gagtttagca |
| 41581 | gataagagta attgagatgt gagaaatgaa ggataagagg acattaaaga tagtatatga |
| 41641 | acagttatca gggcctgtga ctttcaccct ttgaatattt tttcaaaaga ctgtaaactt |
| 41701 | ctttcatatt attttggaag atttgaactt ccttggttga aattattgca gaaattcaaa |
| 41761 | cacaaattta tatggattgc ataaacaata acttaaaatg gattagacac atttttattg |
| 41821 | atggttaatt tcatatgaat ttatattggt ggtcaattat atgaatttta tactgatgaa |
| 41881 | acatgtccaa attgtttcaa ttaaatgagg aaaaaaacac aaacaagtac atggtcactg |
| 41941 | tgaattaata aatactagaa tttatttatt tctttattaa atgtattcat tcagtaaagg |
| 42001 | tcttgggcac ctaacacatg gcagactata taaaaaactg agatttcaaa attgaataag |
| 42061 | gtgactgctg accacaagga gttcacaatt tactggggta agactgatgc aaaacagatc |
| 42121 | attagtatac aacatgataa atgatagaaa cctgtatatg gtctaaggga acagggtgga |
| 42181 | gaagtcctaa caaagcctag atttgatttc ctggagtaaa gtgatgctta ttcagtgagc |
| 42241 | gaaagatgtg tgggaggttg tcaagataaa agaaggagga aaaaagtgac tccatgccaa |
| 42301 | gagcacatta taagccaagt tacagtatgt agaaaacaga ataggggtgg tggtggtggt |
| 42361 | ggtggtagtg gtagtggtgg tgcatgtgtt tctctgtgtg tacatgcatg ccaagaaaac |
| 42421 | aagaaggaat tttgtcacag taaaataaaa catgtcgtca gaagtgacaa gatataMgag |
| 42481 | ctgtacttag agaagcagaa gggtcagaca aagggtggat caaggatgat ccagggggcc |
| 42541 | aggcgctgtg gctcatgcct gtaatcccag aactctggga ggccgaggtg ggtggattat |
| 42601 | ttgaagtcag gagttcaaga ccagcctggc caacatggtg aaaccccacc tctactaaaa |
| 42661 | atacaaaaat tagccgggca tggtggcagg cacctgtaat cccagctact cgggaggctg |
| 42721 | aggcaggaga atcgcttgaa cccgggaggt ggaggttgca gtgagcccag attgtgccac |
| 42781 | tgcactccag cctgggtgat agagtgagac tctgtctcaa aaaaaaaaaa aaaaaaaaaa |
| 42841 | aaatgatcca gggtatcatg atacggaaga aagaagaaga gaaaccactg aggaatgtaa |
| 42901 | gagaacggaa cagtgaaatt tacagctgga ttgaagaaag atgtaactgt aagcagagag |
| 42961 | aagaggtagg gagccattgc acgatctata aaaaaatatt gaaagttctg actgcggtta |
| 43021 | aaaaaaaaaa aagatgaggg tgaacgaaag acacagattc aggaataatt aataaatgtt |
| 43081 | aataaataaa cttggtagaa gttggttttt gattgaacat atgtgatggg agagaagaat |
| 43141 | atgagtcccc aatttctaag ctgagtacct gggaagatga tcgtgttggt gaagccatct |
| 43201 | aaagaaatag agaatggaaa aggggaaatg agattggtag aggtgagcct aagtgttatg |
| 43261 | tgagaagaga tgatgatttg aaagttaagt atataatctg aagttcagag gaatgctgtt |
| 43321 | ggctagagat gtgtatagat ttgagactaa cagcaagtga tgtttgttga aactatgaca |
| 43381 | gtggatgagg tcacccaggg aagatatgtg ggtatagaga gtaaatggcc aaattaaaat |
| 43441 | ttaaagattg gacagaggaa ggtaagttca ctaaggcaaa agagaatagc taaagaaata |
| 43501 | catgaaaaat gagaatattg caatgttata gaagtcaagc aagtagaatt tcacaaaaag |
| 43561 | ctagtaattg gccatttctt gcaaatgaag cacatgagga ttttgactcg cagggcacca |
| 43621 | gaaaggcatg atagaaagca tgcctctgaa ccctttgaaa gctttccaaa ctttaaattg |
| 43681 | ctatcacccc cagaaagatc cagatagatc ttcggtggag gacgttaata tgagacatca |
| 43741 | cctaaacaat tgaataaaat gtagaaaaag atcctcaaca ccaatataat taaaaggaaa |
| 43801 | gaaaccatag aacaattgtg acactagaaa aactataatg atctcagatc tactctctca |
| 43861 | agtctctgag aacttgataa aaaaaaaaac actgttctct atctagttaa aggaagtcac |
| 43921 | tttttttctg agaaaccacc atatgttaag ctgtctttat atagacctta ataatcctca |
| 43981 | cagtcaggaa actaagattc aaggaaattt taaaaaatat atcatgaagt catatagcta |
| 44041 | attaatgatg aaacttttat taaaacccaa tttaaagagt aatccagagc aagtgtcaat |
| 44101 | actgttgaat ctaaaataaa gactaataaa acaatattta aagtgaacat ctttgtaaaa |
| 44161 | caagaaatag aaacataagc caatagggtg accatatttc ccagtttgta caaaatagtt |
| 44221 | ccattttatg cttattgtcc ttgtaattat tagtagcact tacgcccttc gctcctttaa |
| 44281 | gtgttgtggt ttggagaata aatatgtgga cactctacat agcaatgttg tgacatggca |
| 44341 | tgttagagat ctaaggtata cagtaatttg aggacaaaga aaacaactga attgcaatgt |
| 44401 | ttttctacca ctgaggtttc aacaattgca gctgtacctt ttccttctct gagacagcac |
| 44461 | gtcagcattt ccttccattt tctctatttt tagaaacgta aaattcagct ataaaatatg |
| 44521 | gctgttggca gacagtacag acaagagtag aattctgcat aataaatcta tagttgaaaa |
| 44581 | atatggattc tgtgggcatt ctatggagtt cttgctgttg gccatgactt tctactaaag |
| 44641 | cgatctcttt aacaagtacc tgttcaattc ctaattgagc tttcgaaggt aaaagttgct |
| 44701 | ctttctgcct gtaatcccag gcctttggga ggccgaggtg ggcagatcac gaggtcagga |
| 44761 | gatcgagacc atcctggcta acacagtgaa accccatctc tactaaaagt ataaaaaatt |
| 44821 | agccgggcat tgtggcaggt gcctgtgttc ccagctactc gggaggctga ggcaggagaa |
| 44881 | tggcataaac ttgggaggtg gagcttgcag tgagccaaga tcacaccact gcattccagc |
| 44941 | ctgggtgact gagcgagact ccgtctaaaa aaaaaaaaaa gttgcccttt cttgtgactt |
| 45001 | cattcatttt atcccatata tataagtaat attcattgag taaaatgtgt gaatttattt |
| 45061 | tttactgaat ctttgagata attttcaaag atacatggga agtatctttt tcaacagcat |
| 45121 | tataggatat tagtggtaaa agaaagttca gagattatct aatgccaccc ttattcaatt |
| 45181 | ttcaaatgag aatatagaaa ctgagaaagc ataagtgatt tgcccacaat cacagagtta |
| 45241 | attagtgaca gatgaataca tttatggtct cttatgggaa aagacagaat aatggaattc |
| 45301 | ttatggttga agaaacttta aactggataa tttgacacct gtaaaagata ttcattaaag |
| 45361 | acaaatttgt gcctgccaca cactgatttc tgttccaatt ttctgtcagt ctacacttac |
| 45421 | agctagagct ggcaactatc atacaagcag tcaacactta cattcttaaa cacttttttg |
| 45481 | tcaaagtatc atggacatca aagacctcta aacacatgaa tctctaaaga gataaaaacc |
| 45541 | agatatgaga aatctaagtt gtacaaagtt gttcctttga ttcttctttg agccctgaac |
| 45601 | tgacattttt ccctaacact tatgctctga accatatgac agtccatcaa gttgttggtg |
| 45661 | tttgtctgct ttgtcctaat taaggatctc cctgtgttct gctgccttgt ttaaaatgtc |
| 45721 | aacaaaagct ttaaatagca ctgtatctca gtgtgtgcac tcattattaa ttcaacatct |
| 45781 | ggatcaaagt aaatgatatt atatcttact gaaatatgct ttgttttttc ttattaagct |
| 45841 | tggcagttag atgattgtca aaagatgcat gctttattaa aacgctttaa gcaagaaggg |
| 45901 | tatatcttag tacaaaatta gatagaacag gaaatccatt aaggaaatat acaacatttt |
| 45961 | gattttatat ttccttgaat ctctacacat agagttttct aatttttttt ttcttaattt |
| 46021 | gatgactagg acttgttaaa ttttcaccaa ttaattagaa aataaaaatt ttggtaaaat |
| 46081 | gactctgatg aagcacaaaa tgatatgcac ttttattgtt ttatttggct aatggcacag |
| 46141 | tttagcatgt ccaatttttc tctaaagcct tatattctct gtggcaccaa Sagcagttca |
| 46201 | ttaccccaaa atggagctca gtatttgttt cacaaatagt cttaccaaca atcgccaaaa |
| 46261 | taatgaaaat attattgcaa tatgcacaat tatcaaaaga attgattaga tataattaat |
| 46321 | tcctatgatt tctcagtaat gtgaccttac tatgcattaa gcaatgacct ggtttccaat |
| 46381 | taccaaaaac ttcattgcct tctgaatgta gatatatggc attttgtgat gtactatccg |
| 46441 | ttgattatta aaaattgctt cagcaagttt caggaactat gtctgtgttc tcaaacagaa |
| 46501 | attaaataat atactactac agattaaata aatgcctttt aaaagtatga tttgaggaga |
| 46561 | tgtcagcaag atgataaaat aaggctctcc agtgctccct ctctcacaga aacatcaatt |
| 46621 | tgaacaacta tccgcacaca aaaatacctt cacaaaagtg aaggaaacca ggtgagagat |
| 46681 | ttcagcacct gggtatagca cggaaacaag aaaagatgca ctggagagag aggaaggaca |
| 46741 | gtttcacatt acctgcttca cttttccccc aattaccaga cagcacagca tggagacaga |
| 46801 | taccctccac atgatggtag gagaaggaag tgagcatcag actttgcctt agaccccaac |
| 46861 | actgggcctg ccacagaaaa acccagcacc aaagagaccc tcacagccct agacaccagg |
| 46921 | ctagtacttg tggactgagc ttccacacct ttcccagcac tgatcagggg cttatagcct |
| 46981 | tagactcaag gtctatacag cagacttggt ctctgggcca ctctactgcc aggtctacct |
| 47041 | tagcagccca gcctctggaa cttctccagc actgggctgt cccctataat cctggacatc |
| 47101 | agatccatcc cagtaactga ctatttcctg cagactcaga ctcaaagcca atcccagagc |
| 47161 | caagtagccc cctgtggacc caaggataag gccagcctct acagatgtag gcaaatattt |
| 47221 | gcagacatag gcgtcaggtc tgcccttgtg tacccaggtt tcaggcctat ccatggggac |
| 47281 | tcaatcaata ggtccacact tgtggatcta gacttaggcc caaccctata gacccaccca |
| 47341 | cctgctgacc caggcaccca gacaccaagc ccagccaacc tacctgagga ctccaacagc |
| 47401 | aggcctaccc ttagacgaca ccaaatggca tgcacaggtt ctatggaaag gctgactgat |
| 47461 | aaagagtttt cccagacaaa gccagtctgc aaagactgga ataagccctt acttcttcag |
| 47521 | atgcacagac atcagtgtaa ggaaataaga aacatgaaaa gctgaggtga cataaaccac |
| 47581 | caaaaaaata caaaaatctc ccagtggctg agtcaagaga agtggaaata tataaactaa |
| 47641 | gagacaaaaa agtcaaaata attgttttaa ggaagctcaa caaacttcaa aaaaattaca |
| 47701 | gggaaacaat ttcataaaat caagaaaaca ataaataaaa caaatttaat agagagattg |
| 47761 | aaattatttt tcaaagacaa agagaaatcc tattgccgaa tataatgaat gaatgaaaaa |
| 47821 | tgcaatagta ggattgatga aggagaagaa agaatttgta gccttgaaga caagttattt |
| 47881 | aaaaatatat ggtcagaaga gaaaaaatga aaacaaatag agaaagttta tgtgatttat |
| 47941 | gggagagcat caaaagagca cattttttag ttcaagaaga gaaagacaaa ggggtaggaa |
| 48001 | gtatgtttaa ggaaataata acagaaatgt tttcatatct tgggaaagac atattcacgt |
| 48061 | agaggaaagt taaagttctc taatcagatt caattcaaac aagattacac caagatatat |
| 48121 | cataatgaaa tcaacaaaaa tcaaagacaa agaaaggatc ctaaaggcac caagagaaaa |
| 48181 | gaagcaaata acatttaagg gaatgttcct atcaagctag cagatttttc ggcagaaacc |
| 48241 | ttacaggcca ggagtgtatg ggacaacata atcaaaatgt taaaataaaa taaaaaaact |
| 48301 | atcaattagg aatactgtac cccacaaagt tgtccttcag aaaaggagag ataaagtctt |
| 48361 | tccacaacaa aagttgaggg agttcaccac cagacctgtc tcacaagaag atgcttaaga |
| 48421 | gagttcttca ggctaaaaga aaagcattct aattagtgac acaaaaacat gtgaaagtgt |
| 48481 | aaaactcact tacaaagata agtacacagt caaactcaca acaccctaat actgtaatgg |
| 48541 | tggtatgtaa ttcacctata tctttaccat gaaggtgaaa agaaaaaaac tactaagaat |
| 48601 | aacaatagct acaataatgt gttaaggcat ctacaaccac ctgatcttcg acaaacctga |
| 48661 | caaaaacaag caatggggaa aagattccct atttaataaa tgatgctggg aaaactgcct |
| 48721 | agccatatgc agaaaactga aaccagactc gttccttaca ccttatacaa aaattaactc |
| 48781 | aagatcgatt aaagacttaa atgtaaaacc caaaaccata aaaaccctag aagaaaacct |
| 48841 | aggcaatacc attcaggaca taggcatggg caaagacctc attactaaaa caccaaaagc |
| 48901 | aattgcaaca aaagaaaaat tgacaattgg gatctaatta aactaaagag cttctgcaca |
| 48961 | gcaaaagaaa ctatcatcag actgaacagg caacctacag aaagggagaa aatttttgca |
| 49021 | atctactcat ttgacaaagg tctaatatcc agaatctacc aaatttacaa gaaaaaaaca |
| 49081 | aacaacccca tcaaaaagtg ggcgaaggac atgaacagac acttctcaaa agaagacatt |
| 49141 | tatgtggcca acaaacatgt gaaaaaaagc tcatcatcac tggtcattgg agaagtgcaa |
| 49201 | atcaaaacca cagtgagata ccatctcatg ccagttacaa tggtgattat taaaaaggaa |
| 49261 | acaacagatg ctggcgaggc tgtggagaaa caggaacgct tttacactgt tgctgggagt |
| 49321 | gtaaattagt tcagcaattg tggaagacaa tgtagtgatt cctcaaagat ctagaaccag |
| 49381 | aaataccatt tgacccggca atcccattac tgggtatata tccgaaggat tctaaattat |
| 49441 | tctgttctaa agacacatgc acacatatgt ttattgcagc actatataca atagcaaata |
| 49501 | cttggaacca acccaaatgc ccatcaatga tagactggat aaagaaaatg tggcacatat |
| 49561 | acaccttgga atactatgca gcaataaaaa agaatgagtt catgtccttt gcagggatat |
| 49621 | ggatgaagct agaagccatc atcctcagca aactaacaac tagcacagga acaaaaaacc |
| 49681 | aaacactgcg tgttctcact cataagtgag agttgaacaa tgagaacaca tggacacagg |
| 49741 | gaggagaaca tcacacacca ggacctgtca gagggttggg ggcaagggaa gggagagcat |
| 49801 | taggaccata cctaatgcat gctgggctta aaacctagaa gctgggttga taggtgcagc |
| 49861 | aaaccaccat ggcacatatt tacctatgta acaaacctgc acgttctgca catgtatccc |
| 49921 | agaacttaaa gtaaaatttt aaaaaaaagt gttaagggat acacaatata aaaatatgtt |
| 49981 | aattgtgaca tcaaaaattt taaatgtggg aggaatggtg gagtaaaagt atagtttttt |
| 50041 | aaaaatgcaa gcaaaaagtt gttatcagct taaagtagct tgttataact ataagatgtt |
| 50101 | tttgtaattc tcatggtaac cacaaagtaa aaatttatgg tagagacaaa aaagatacaa |
| 50161 | agtagggaat caaagcatac caccagagaa aattatttta tcagaaaaaa aagacaaaaa |
| 50221 | aagaggggaa gaaatgaaca aagaacctac aaaacaacca gaaaacaatg agctaaatgg |
| 50281 | cagtattaag tccttaccta ttaataatta ccttgaatgc aaattgatta aattttccag |
| 50341 | tcaccttgga tgtgaagggg gctgagggaa tagaaagaaa agactccaag gtatctcatt |
| 50401 | atccagatga aaacctggat agaaagtagt gccatgaatt ggaaggagga agcatagatt |
| 50461 | tggtgtRgag aggagatatg gccagtttaa tatttaatat gttgattttg caatgccaat |
| 50521 | ttcaattcca gtgactgtta gatttataca tctggaactc aggccagctc tatgggacga |
| 50581 | tgtgtagaat tgagccattt atgataatta aaattaaggc tgtggtggaa gataagactc |
| 50641 | ccagagagaa aagaaataag ggctataaag aaaaaatttc tgagatgcac aattctaagg |
| 50701 | gaagagagaa caacccacat tgagcaatgg aggctgcagg agaacaggtg tgctgccacc |
| 50761 | acagaaatta acatgcaggg gtgcagaaag gaaggtgttg tcagcttatt caaatgggca |
| 50821 | aatacatgag atacccattt ttttggactt ggcacctaag aggccactgg tgaatttaga |
| 50881 | gaaagcagtg tcaatgttta aaaagtgaac tggagtaatt gtgagtaaac aaggactaat |
| 50941 | gttttataga gacttggcta taaagctata agtcatactt ccattgctaa aggtagaagt |
| 51001 | gtagttaaaa ataaaggttt gctttgattc tttgttcttt gaaatgaaag agactagaac |
| 51061 | atgtttattt gtaagaagta attgccactg gaaactgaaa cataggacac acacagagaa |
| 51121 | ggaagagacc cacatgcaga aaacaaaggc tataaataaa agagtacagt gtcctgggtt |
| 51181 | ttcctggatc ttgtgtaaat gactccatca cagcattatc atgttttctt ctcaacatct |
| 51241 | tcatcattct actaccattc gtagaaagtg gattttcata tattatctca tttagtcttt |
| 51301 | gtgacaaacc tcctattcca agtatgcaac tgaggcacac aaagatttaa aaatctatga |
| 51361 | gttagtagca gaggccatat ttaaacccaa atctaactga atacaatgtc tgtgttcttt |
| 51421 | gcatttttcc accccaggct tatagttcct actaattcaa cctattgaaa tatattttaa |
| 51481 | cgttttatta ggcaaacaaa gcaacagaga acagcaggga aaaaggcgac tttatctcta |
| 51541 | gctgacgggg ctcttgaaat gctgttaatc aggattctag atacaaattt ccacgtccac |
| 51601 | tcagaggggc gagaatgtgc aatatattgt gatgctgcca ccttgtggca agaaaaaatg |
| 51661 | aaaacattaa gaggtgcttt gtgtgcatat ttatcatttt caattaaatt gctctagttt |
| 51721 | atggttcagc tacggctgca cattgaaatc atctgggagt tttaaaaata ctgctgccta |
| 51781 | gatcccacct cagagattct gatttaattg ttctggggtg aggccttggt gtcaggatat |
| 51841 | ttttaaactt cccaaatgat tctaacatgc agcaaagatt aagacctctg ctctaaccac |
| 51901 | ttatacagta gagaagcaag tcttgtcatt tgcctagtta tgtttcattt gtatagacgc |
| 51961 | aaaagaaaac attctagaga aagtaggtga ttttctctca atctttaatg gcatctagtt |
| 52021 | cttggtagaa tacaatgatt tctggacaga tatatcatat actatgattt taaagaacag |
| 52081 | gacaaagaaa tttgtgaatt gtaatcaaag tggacacaag atttgtgtct cataatcaaa |
| 52141 | tgcagtccgt agactaaagt gagcagggag aatctctgag cctcataatg cgtcacaggt |
| 52201 | tttctgaaag gaacaatgga aagagcactg aagcaccatt gactctggca ctagtaagat |
| 52261 | gagtgacctt gagcaagtca ctcagtttgc aatgaaagat tttcatctac tgctttattc |
| 52321 | aagagaaact agttccaaat gttcaacaaa tatcatacca ggtaattcaa caaaactttc |
| 52381 | ttaagtactt gcaatgcgaa aagcatggaa ttataatgtg ttataaatgg aaagcactaa |
| 52441 | tgttgcttag agacatcata gcattaggtg accagaggaa cctttgaaag ctagtaatgc |
| 52501 | aaattttaat tatgatctga actgagtgta attagaaagc ttaattacat aatgctccag |
| 52561 | tggtttggca aagaagttga attcagtttt ctctattttt cattctgttc tgtagttttc |
| 52621 | tcagatttat ttattagctc tacaccatca aaaagtgtaa tcatgtcttg aaaatgaaat |
| 52681 | ttctctttcc tgctccttgg aaattaaaat tttctatggt tctttaactg aaaagtttca |
| 52741 | gtaattgcac caccaatcac ccagtgcctt ggtcaggaat ctttctttct tcattcgttc |
| 52801 | atttaacaca tatttattaa ccctctacca tatgatatta ctcatctttg aattccacct |
| 52861 | cccttaaatt ccaaatttaa ttatttacaa ttcatgtcaa ttattagctt ctaaacatct |
| 52921 | cttgaatatt ctgYccatgt ctacagtgat ttagattact acagatctat cctcctgcct |
| 52981 | tctagctccc ttttctcaac tccagtccca cactgcagac agtggccctt tgaaacttca |
| 53041 | aatctgctaa tgctattgcc ccacttccta gtgaatggct tcccttcacc ctaggatagc |
| 53101 | atgcatggta catgacaatg cttgggaact ccttccttat ctggtccctg cttaccagcc |
| 53161 | caaaataatt tctcaccctt ctccattaag tcactcagaa agcttaaagc tttgcaattc |
| 53221 | tctccaccct ctcttgaccc tgggattttt gcatataccc tttcttctgc ttgcctttct |
| 53281 | tcacttggat aatttctttt ttagcccttc atgttcctgt ttgaaaatta atcactccaa |
| 53341 | gcagccttct gtaacccaca ttactgggtt atttttatgt gtttccaatg tacaataagc |
| 53401 | ttaaaatttg gaatacgtat catcaacatt tacttattta tcttctcact agaccatggg |
| 53461 | acaaagaatt gagaataaaa gacctaggaa tccaacttac aagggatgtg aaggacctct |
| 53521 | tcaaggagaa ctacaaacca ctgctctcta tttgtctgtt attcgtgtat aagaatgctt |
| 53581 | gtgatttttg tacattgatt ttgtatcctg agactttgct gaagttgctt atcagcttaa |
| 53641 | ggagattttg gactgagatg atggggtttt ctaaatatgc aatcatgtca tctgcaaaca |
| 53701 | gggacaattt gacttcctgt cttcctattt gaatacgctt tatttctttc tcttgcctga |
| 53761 | ctgccctggc cagaacttcc aatactatgt tgaataggag tggtgagaga gggcatcctt |
| 53821 | gtcttgtgct ggttttcaaa gggaatgttt ccagcttctg cccattcagt atgatatttg |
| 53881 | ctatgggttt gtcataaata gctgttatta ttttgagatt ttgggctgag acaatggggt |
| 53941 | tttctagata tacaattgaa aatctagaag aaatggaagc acagtatata gtacatagta |
| 54001 | gatatccaac aagtatgaac cattgatgaa gaacaattat ttgcattcaa aaatctacga |
| 54061 | tatattaact cggaaaaatt ggtcaggtaa agagatgaaa taaattatga catgtctgac |
| 54121 | attactcaaa taatttttaa aaagtagatt ccagcttttg atatacacat attagccaca |
| 54181 | caatcatggg caatgtgctt aacatccatt aacatcctga gctttagtgt cttcaattct |
| 54241 | aaacacagat acattcattc agcaaatatt gattatatga ctactaaggg ccagacagtg |
| 54301 | ttctaggtag ttgggataca gcagtgcccc aaagagtcac tgtctctctt tttacgcatc |
| 54361 | ttaaattcta gttgatgaga cagataataa aaagagatat ataatgtcaa acagtgacac |
| 54421 | atagagaaaa aagattaaga ggctagagtg taacagagta ctattctatt ttttcttttt |
| 54481 | agtttttttc tttttgtttt tgttttactt tttaaactat tattagtatt atttttacaa |
| 54541 | acacttgtac caagggctga ctttcaatag atcacagcaa ggatcccctc tgctacatat |
| 54601 | gaaaccctga cccagaagca ggtcatctac ttatgtttta gtgccaggtt acacacgaat |
| 54661 | gtgtgttaca tgacgggcaa gggggcggcc gaagagtact attttagaaa ggatggtcat |
| 54721 | aaaaggcttY tttgagaagg taacatttga gcagagattt gaatgaagta aaggagggag |
| 54781 | caatgtgaat atctggagga agaatgtccc aggcagaagg aaatacaatg caaagaccct |
| 54841 | aaagggggag cttgcttggt gtggtaagga aaccgcaagg atgccatata ctggtgcaga |
| 54901 | gtggaaaagg gaagcctaaa ggtgatatca gaaaagtagg caggagccag actaagtaga |
| 54961 | gccatgtttt ccaaactgca gtcatgaaaa caaattggtg tcatcactag caccttttag |
| 55021 | tgtaactaag aagagtaaaa tgagatagaa aatatcagtg tatggttttt cttttgtgtg |
| 55081 | tgtgtacgtg tgtgtgtgca cgcacgtgca cttgtacacg catgctgggt tgcaatgtaa |
| 55141 | aacacccttc ttactatggg ttatgataaa atttgaaaca acactgtggt agggccttgc |
| 55201 | aagccatgat aagtactttg agttttgtta taaatatgat aagaagccat tggaagatgg |
| 55261 | gaaggtgtta gctgaggaat tatgtgaatg taacgtgttt tacattttaa aaggaaaatt |
| 55321 | ctaggcattc tgcagaatgg accacaaggg acaagagtgg tgctgagaga taagtcccag |
| 55381 | aatattacat ggtccagggt agaaatgatg tttgcataga tgaaaggatg agaagtgatc |
| 55441 | tgagtcaagg tatatttaga aagtaagttc agttaagagt tgttgaggat agtcatcatt |
| 55501 | ttctagagtt tttatgaggc ttaaataaga ttggatatca gaaagggttt tataaacact |
| 55561 | agagaagtgc tcagcagagt acagctgagt tcaattcaaa gggcatttac tgaccataca |
| 55621 | ctatgcacct gccattactc tcactgtttt ctctgcactc ggtagataaa actgaatttc |
| 55681 | tgctcccaag gaactttaca gcagtagagt tgtggaagat agacataaaa atgaatcact |
| 55741 | gcagcataat gcacaatatg ataatggcat atggaaggtt ctaaagaaaa tgagttaact |
| 55801 | taaaattctg agaagcagta gcttttgttg caagcctttc ttttcatagg taaaagatca |
| 55861 | cattagcccc aaggatattc tgtaggacaa atatgagcta aaaatgaact agaaaatagc |
| 55921 | tctaggtgaa aaacacatcg aacacagata ttgctgagtt agtctattaa agccacatgt |
| 55981 | catttgcact tttgccctac agtcacaaaa caaaagcaaa tgaacaaggg aaagaggatc |
| 56041 | ttcagccctc ctacataagt aacatgaatc aataaggagt catcctggag accacagaaa |
| 56101 | accacactct tccagttagc atcaatgaat agccccagga tacaaatcca cagtgaatga |
| 56161 | agaaacatag ctacagaatt agaaacatta ttaaggaaaa ttgatttgag tatacttcac |
| 56221 | taatgttaga gggataatat tatattttag gttgaaatgt aataattttt ttaattccaa |
| 56281 | agtgtctatt aattttaaaa tatacatact ttttgttaca aatatttatt tttccccttc |
| 56341 | ataccactaa tctctgtgaa aaagtttaga atgtgacata aaggttctag gttaatgaag |
| 56401 | tctttcaatc aagcataaag acttctgcta atctagtaaa aaccgcccct tctacctaca |
| 56461 | tggattcaat atatttgttg cattatataa gaaagcattt gtgtaccaca tttacatatg |
| 56521 | agatgtttat cagaaagaaa gtggtgctct gaggagaggg agatggggaa cagaatcact |
| 56581 | tgagagtgag gatttatttc caattaatta acatgcccac aagaatctgc cataccccac |
| 56641 | atgaagaatc attgctttaa tatagattga tgagttgata acgttaatga gttgatgact |
| 56701 | gtctctcagg tatgttgggg aacaaaaaag tttgagaaac aaatacagtc ataaagaact |
| 56761 | atattctaat aataaggcgt taccattgct cctacttttt aatgtattat atacattatt |
| 56821 | atataatact ttttaatgta tttaataaat atatctcata gtggattttc ttaaaacaga |
| 56881 | gcttgccttt tcattaagta attactccaa ggagtaattt taaaagaaaa taagtctaac |
| 56941 | tgagcttttg tgttaacatg ataggaactg ataacatttc catttttctt atgtcacata |
| 57001 | gactaaaact gttcagttta aaatagttca attacctcca taagctttaa gaaaaccttc |
| 57061 | tttaataaca tcctatgttt tcaaagttaa ttgggtactt tcacattttt ttcctttaaa |
| 57121 | acattattag acaggacatt tcatgggtaa agattagtga tgcaattata ccaccatctc |
| 57181 | tgaatgtcat tctttcttca tctccattgc cattgaagtc cattttgtca tcttttattt |
| 57241 | caactacttc actagcttcc taaatcatct ttctactatt ttatcacttc atcagtcaga |
| 57301 | aagaatgatc tacctaaaac acgaatccca ttgtgttact ccctgattaa aatctgttcc |
| 57361 | cccaagcctg cagcttgggt accctctggg aatgacttaa aaggtgtttc ctgaccagga |
| 57421 | ccctgcttac atttccagct gcatttctca ccattccctt ccctgctcac agtgaattaa |
| 57481 | ctcatagttc cccaggtgtg tcctggttca tcttctctcc tggccattga gcaggctgct |
| 57541 | tcctttgcct accagattct actcttccac ctccatttca cttggtaact cttgcccatc |
| 57601 | cttttggtcc catttgaggc ttttcttcct cctaaaaacc tcctatgtct gctataggat |
| 57661 | gagttatgcc ttattccttt gtgtttctat ggcactagta ctatggttat agtatcttac |
| 57721 | ttacttacat attaactcta ctacaaacta ctaaataaca gaaactatgt ctttttatca |
| 57781 | ttagaattag tcaatcttca atggcagcgt acacttgaag cttaacaaaa gtttaagtga |
| 57841 | atgaatgtgg tggacctgtc caaggtatga tgagactttg agtccatgtc ctctaattgt |
| 57901 | tgacccacag tggtttccac tgtgctctac tgtctcccag tgccatgacc ttggtctcca |
| 57961 | cactctgtaa ctcttggtcc tcctcccatt gtctgtctac agtcctttgc tttgggaaag |
| 58021 | ctagactaag taaggcaata ttttaaatta actaattgca agccaagttt tttgcctgga |
| 58081 | tttgagctta gccttccatt cttgggggag ctattcatta cttaagaaaa cctacctgtg |
| 58141 | actatgcatg taagaattct ggctaggaat aaactattgt tcatgattaa acaggaatgg |
| 58201 | tgcaagaggc atgagcaggc ctacctcaga aaaagaagtg gtaacagatt gctgtactat |
| 58261 | gaaaacagca ttctggcata taYgtcctga aagaaagtca ccaaaagttc tttacgctaa |
| 58321 | cttagtcaaa aacaacacat accttaggta agaaattttc agaaaaagtg cttgaggStt |
| 58381 | agctaattta tctctcactt tttacagagg aacatcgaag gaattcagct ttcaaaatgc |
| 58441 | acctaaagat agaaatcaga ttcttcttgg aattagaaac tgtggggagc atcttttgat |
| 58501 | tgttgattga actcatgttg ttttacatac attatattct ctttcacaat atattgtggg |
| 58561 | tcattattct acaaacaacc ctctgagtct catatgaaac ctgcagcaag ttatggaagt |
| 58621 | ggcttttatc attcttacaa ttgccctacc attagaaagg gacataataa ttttattata |
| 58681 | gcacatactc ctggaaagac cataaacttt taatagactt gtaaaaaggg agtgtaacct |
| 58741 | tttcaatatt ctgtctcatc tattctacct tctcctcaca tataacgaat gccctagggc |
| 58801 | ctaagtcatg gatctacact tgggaagaaa atgttgagat tcagcctttt ccctgaatct |
| 58861 | gaaggttacc atgatttagc acccaggaat agtttctgcc tatccttttt ctggttcttt |
| 58921 | tgcagctctg caacttccag acttgtggtc tatgtagagt tgtgatgaca ttccgacttc |
| 58981 | atgtctatag ctttcattta gggattttta aatgtcagca taaacatcct acagcgttta |
| 59041 | aagttgtctt cacatcacca cacatattca ttttcagcat cattaatgat tacactttct |
| 59101 | gattattttc ctttctctgc tagactgtca aacttcttag tttacttcct aatattgata |
| 59161 | gcatcaccag ctctaagcat gacttctcaa acaagatgtt tggttaattt tctgtgtgtt |
| 59221 | tttgttgttg ttcatttgat cgtctcaact ttttgttgca atgcacaaca taaccacctt |
| 59281 | cttagatcat ggacaaagaa acaaacaaaa atctttggag acaaacatgg cagaggaaaa |
| 59341 | taaatatcgc aaaggaagtg agtgcttccg aaaaacaaag tggaaacaaa gtatgaggaa |
| 59401 | cccctgaaag cctcacagca taagaaaaga actagctact tgaagagtag acagtggcta |
| 59461 | ttaatttcta tttcttgaaa aaaaaatacc attttaaaat tgtcRgggac cacaagaaac |
| 59521 | tgtttttata atgttaatag tttttttaaa tcacaataat aaaaattgaa agcataatat |
| 59581 | tttacattta tcagttactt ctgcctccac cactttcctt aaaaaaagta ggacttttac |
| 59641 | agatgaaaga gcattttaat acaaatttta aaattacccg aagacaataa attatttcct |
| 59701 | aaaacgtgta ttaaatcttt ttgttatgtc tataaatgat aatctagtct aagagtaact |
| 59761 | tatcagcatt tttgcagtcc cctcccccca tccactgaaa actttcttta ctagactcat |
| 59821 | gattttcaaa attttaagaa ttaaatttgg agtaaaggaa ctccgaagat ctgaaaaagc |
| 59881 | ctgtgaactg gatgtccaga aagatgtaaa tacataatta ttgatagaaa attttgggat |
| 59941 | gtttttcaaa agcccccaga agttcctcta aggaaccctt aggggtcata ggaaagctca |
| 60001 | gtgctgggta gtgagctcct aagagcagag aaatgccctc ctgtgtgcat ctctagatct |
| 60061 | ggtacagttt cagcattcat aggggtgcta taaacatttg ttacgttgaa tcatggagga |
| 60121 | taagtatgta tatactcggg tgttgttgtt gctgttgttt gagatagagt cttcctctgt |
| 60181 | cacctaggct ggagtgctgt ggtgcaatct tggttcactg caaactccRc ctcccaggtt |
| 60241 | taagccattc tcccaccaca gcctcccaag tagctagtgt aagccaccat gcctggctaa |
| 60301 | tttttgtatc tttagtagag acgggatttc gctatgctgc ccaggctggt ctcaaactcc |
| 60361 | tgagttcaag tgatccaccc gccttggcct cccaaagtgc taggattaca ggcctgagcc |
| 60421 | actgtgcctg gagggtattt tttttttaaa aagaagaatg tagtatttaa aagttcatca |
| 60481 | tgcaattaaa cctcattgat tttgagagca aaggagcaaa gaaaaatctg gcaaatagca |
| 60541 | aacctactac ttaaagtaaa cctttaggaa cttaagcagg gctgctcacc aaataattct |
| 60601 | gctgggtaca gaactgatgt ggcctttaaa ttacagagtt tatgtataat aattaacatc |
| 60661 | caggccttgc agaaaacaat attacctctg cagagcatat cattttcttt attgataaat |
| 60721 | gctctttact gaaaacacaa tgatatgagg atgggtagga gtgccttaga gctctccttg |
| 60781 | aacatgtata cacagtggtt aagaggagat tctttgatga tggacaaaaa ttcaagtcta |
| 60841 | gtttgattgt tttagcaaca ttacaggtaa gcaaacaagg caggaatgca aattaccaaa |
| 60901 | agagttatgc ttcctagaga ttccccaatt ttgtagcctc tacttatctc gaggcaaagc |
| 60961 | tgtctgacag agattctgtc ttctgacata ctcctattct tttctctttt ttattcttcc |
| 61021 | tcttagtata aaatttggta aattttagtt aacagaacat ctcacaagtt cccaccctga |
| 61081 | cttctttgct tatttcatag aaggaatSct aaaaaagaaa aatatatatt tttttctccc |
| 61141 | acagtttatg gattacatcc tatgcagatg acacgttgga taatctcttt ttttccagga |
| 61201 | tgaaacatat taagaaaata aaatacactc tcacaaatgc ccactacctc aagataaata |
| 61261 | ggtagatctc acaatatctc agatatcacc taggagagaa gaaataaaat caagtatttg |
| 61321 | gaagaacctg acagaatacc ttagccatac ctcagccttt cagagagcaa caggcatgaa |
| 61381 | atgttttaga aaaataattt ctgttccctt cccccttttc ataaagtatt ctctctcagt |
| 61441 | gatccattca aaagattaat agcactcatg gtctaaagtg ttcaagataa aacatgaaaa |
| 61501 | caaaaaccaa agcatggatt ctactagaca acaaaacatg tctctcctaa gaatgaacaa |
| 61561 | ttcaaccatt ccagcttttc acacctgaga atcttggatt cagtggattc tgaatatccc |
| 61621 | aatgaattaa aattaagatg aacctttctc ttcaggaata gaacttgcta aaattagaga |
| 61681 | taaatgagtt acatattttc agtgaaatac atattcctat tataagtcat atagggagtg |
| 61741 | aggctcctaa aagcaggtgg ctaactctat ctacaacaaa cattttgaca agctcccacc |
| 61801 | aacataatac atggcatcaa aacaatccga aggcaattca aacgatagct tcacgttaag |
| 61861 | cagctgaggc actgggcaag caaaatctaa tgaagctata ttttctctgg aaggcaatta |
| 61921 | gtaaactgct catattttgc tgctgaaaga atacatcagc atgaacagat ggaaatttag |
| 61981 | aactccacaa atggaattac agacattgat ttaactttcc ccaagcaaat gaaaaccaca |
| 62041 | aaatacgaat tgctctttct ataattaagc acataattag agtgcatcag attcatatcc |
| 62101 | attgtagctc atatttcttc ctcctcattt ctaaatttat atatattctg agtctaacaa |
| 62161 | acctacttag agttcaactc cacactgaaa gagtcttcta tgacagatag atggtactaa |
| 62221 | actatttctc aaattaagcc tgccatgggt cacactagct ttttcacagc atgacacatg |
| 62281 | cggcaataat aagaaatcca aagactcaaa gaggtagtcc ttgtggcagg acagatggag |
| 62341 | agaacagctg agggaggaat tcagccagtt tattaactcc atgtgagtag aacaaatcat |
| 62401 | atttaaaaga attcataagg acatctggtc gaaaaacaga atttaaatga tggcagtttt |
| 62461 | agaatggaga aaagagaatc atgatgtctc acagtacaat atcactgagt tacctctttg |
| 62521 | tttttctact tccaggaaac aaaaggtcac cagtgacctg cacacagatt atagaccaaa |
| 62581 | ctggtgRtgg gcacttggga aatacttgga ctaattgtca agtttaaaat atacaagaaa |
| 62641 | tactaaaaca tattatggaa ataaaatcac taacatgcta aacattacag atacttgaaa |
| 62701 | ttattcacat tgttaattat tcttttcctc attctaggat acagtagttt gtcttacttg |
| 62761 | tgaaaatatt taggggaact tagataaaat ttttgtttga tattcctaaa atgctagaaa |
| 62821 | tgacctcaga aatctttttg agaataagcc ctttataaaa acctccaaat aggtctttcc |
| 62881 | tgagaaaact caattgatgt gctttatcaa gtataatagt atgtttcatt tataagcttt |
| 62941 | ccaagttgaa atccaaagta ataagcatgt ttcaaaaaaa atctaaacaa gtaaaaatgg |
| 63001 | ggaaacttac taaaactgtg cacacatata tagcatcaag aaataaacta gcagtaaaat |
| 63061 | ccaaggtcat attcagtgtg ataggcactg cagatctttt ctattaataa aaagcaacag |
| 63121 | aggctttttc taYccatgaa atgttttttt aaaagagctt tcttattatt ttagtgaaat |
| 63181 | gaagtttttc cttcttaata aaaattttca aatttctacc aaaaaaaatc cttaagtaac |
| 63241 | tcaagttttg cagtttatgc ttatttaaat atttgatagg tattttctgc cacttgagag |
| 63301 | ttgctattaa attttatata ataaacttca cttgcaaact gacttccttc tcagacaaat |
| 63361 | caaatgacag caaacaaaaa taatgcctat taactatttt aagtacatta ctgtaccctg |
| 63421 | tggggaaaat caggaaacaa aaactgtagt ttcagaattt cagatggatt agttattcag |
| 63481 | aaattgtctt gaggacatgc cctaaccttt tgatatcact catcaaagga aaatgtgact |
| 63541 | cacaaagact ttaatcccct ttcctcccaa cgggagatac agtctgtata gagagctgtt |
| 63601 | tgcatttata actcaYttaa atcatgtctt ccagatactt caggtttaac tcacagtgct |
| 63661 | acactcaaac taaccaattt aataaaagga ttataaaata tttgaagcaa acttatgcat |
| 63721 | attttattca atggtttatc tccaaacagt agatatctac tgatgaacta tttgaacatt |
| 63781 | agatgcatca aattcatatt cattgtagct catatttttc cttatcattc ctaaattcat |
| 63841 | atatactgag tctaacaaag tacattgctt ctagttataa tcaaatatat ttataagtct |
| 63901 | attcttcacc atgttgtttc aggaatttat tatggattca agagaattca gtaaaagaca |
| 63961 | actggttaag ccacagttaa taaaggtaga aacaatgcaa acaagaactc attcatggag |
| 64021 | tagatttaat agagcaagta acacaggata ttcataatca aatgatatta gcttttacag |
| 64081 | taaaaatgta tttgagagaa ttaaaagtca gtgataataa ggaatgagaa taactccttc |
| 64141 | ccatcatcct cagtcatgtg aactatgctc aaaggcttca gatggtcaat aaattaatac |
| 64201 | atgaatcttt aaaatataaa ttaaatattt ttatttagtt gctatagtaa atagaaagat |
| 64261 | ttaatttata gtttaaagat tgtatacttt tatgtttagt atagcaactt taggattctt |
| 64321 | tggttttcag taataatcta tgcatttggg taaccaaact tagtcagctt ggtcaggact |
| 64381 | tttttgttta tttattttat tttattttat tttattttat ttattttttt taagacggtc |
| 64441 | tcactctgtc tcccaggctg gagtgcagtg gtgcgatctc ggctcactgc aacttctgtc |
| 64501 | tcccgggttc aagcgattct cctgcctcag ccttcccagt acctggcact acaggtggct |
| 64561 | aatttttgta tttttagtag agatggggtt ttgccgtgtt ggccaggccc aaactcctga |
| 64621 | tatcaagtga tccacccgcc ttggcatccc aaagtgctgg gattacaggt gtgagccact |
| 64681 | gtgcccagca ggacattatt ttatattcag tcaaataata gtttaaaaat catagctaaa |
| 64741 | agtggataag cataacttgt taattatgat tcaaagcagt tgatgcacat ctggttgaag |
| 64801 | attagcagat tctcactgca gtttagttat aactgattaa catttcaagg gacataggat |
| 64861 | ttcagattct acagtcagga aaccatacac caaacaagtt aaataatcaa gtaaggtaga |
| 64921 | taattcatat ttggaggagt ttattttgtt tcttcaggag atgataagtt atcaaagatt |
| 64981 | ctcattcaat agaaattgct attaactatt agaaacctac caagaacaat aaaagcaggg |
| 65041 | acctgttttt ttttcatttt ttgtttaaaa attcaatgct gaatcactat catctaggtc |
| 65101 | agtgtggaac acaaagtgtg ggttcagtca atatttatag aataagtgaa taaatactaa |
| 65161 | actgttaaca aatagtcaaa gcttatgtga ataactctgt tggataaaaa agagaaaagg |
| 65221 | gtaagaagtg aagaagagat aaaaaaggaa gaaataaaaa agaggtgagt gagaggaaag |
| 65281 | tgaaaagttc tttcctagtg aaagaggtct ttcctatggc agcagaagag tagatttaca |
| 65341 | gcaacctaaa agaaaacaga tggctgaatc ctttcaYtcc ccttacgtac atgtggtcca |
| 65401 | acattgtctc aacaagactg tagccctaca accaacaatc gMaagagtga catcaacaca |
| 65461 | taaaaggaag gaagtgcaca ttcagtggaa accccaatga gaaatacgct tctgccaaga |
| 65521 | ctatcaggtt ggtatagtac ttgcgatKtc ttggcaagaa atcatgaaat acttgaagaa |
| 65581 | attaaataat tattaggggg atgcttactt cactttacta ggggaggtcc ttgagtacct |
| 65641 | cttctaacat ctaaagtctt ctcctctcca gaggaattgt gctccgttcc ctggttactt |
| 65701 | ctcctttact tagggatttc agaattgtga ataaggaaag aatgacctgg taagtgccag |
| 65761 | ccctggacac atttgcccac agatgctgtc ctcccccttt ctttagttgc tctttttctc |
| 65821 | agggggctgg actctattag gccatatttc ccagatttgt ctgtcagttg ccttaggKtt |
| 65881 | ggtctcagcc aataggaggg agatatcacc aggagacaga ggggagaatc taggccattt |
| 65941 | ctccccaccc ttttctctag aagcagctgt atcttacctg tgttagtgac tctagctccc |
| 66001 | attggacagg ccattactac ctgggctcca gtaaatcgcc ccctagctct caggttggta |
| 66061 | gtggcttccc atttttgctt atctctgtgt tggtccaaga gtgtctgctt ttgacttctg |
| 66121 | cattgcaata tgagaacatc cttatctctg acacctttaa atacccctta gggtctcatc |
| 66181 | tacttagatc tctgatctta gaccacatgt ctctcctgac tRcccagctg gtcttgactg |
| 66241 | cttggcctac atatctgcat cagctgtctc tctggaatgc ccagatcaag ctctggaacc |
| 66301 | tgttctactt tgaccacagg cttctgattc ccttctctct ttcttctgat cccRtctagc |
| 66361 | tctattccat tcacttcatc atggttgctg gtcctatagc tgctcaaagt tgaaaagttg |
| 66421 | ctactctgaa tgctgatgtt tgctgaactg caggacaaaa ccactctata attatctcta |
| 66481 | agttcccact tcagatttac cttgaataga ttttaggcat aataggcata cacaatatca |
| 66541 | aagtttacat atttgtgtca caagaagatt aaaaataaag tatatttccc acatgattgg |
| 66601 | tcatcccttc catgcatcct ctttggtaat atccagtaag atataatggc agacaagaag |
| 66661 | atggataaaa taatgtcaga tagtaactct gctcacaggg catggaatct atgatggcct |
| 66721 | tgtaataaaa tagcttaata tagccaggtt aacttgtttc tataaattct ttcaccgtag |
| 66781 | gcaaaaataa cataatccct aaagtagtga actagtaaat aataataatt aagcatttaa |
| 66841 | gaccctctta atcactttac attataaaca aacagtaaat gtgtaatagt gttgactatt |
| 66901 | ctgggctcat gtttcttaag tgtttatttt aattgaactt cagcactaat acatgtaact |
| 66961 | gttactctgt ttcccaggtt atactctccc tgggaatctg aagatcatgt tgtttaattc |
| 67021 | aggatctgta tcggtagatt aaattcccac ccaaccctgt cccaattccc aaattaatct |
| 67081 | tgagttaggt gaaaaaataa caagcaagct cagcactggc cctgtttacc aaatcacctt |
| 67141 | ccagccaaat tggttgaatt agttacacct tttctcttag tctagtggag gatgtcgtgt |
| 67201 | gtccttagat tcccaaacaa aacWtgcccg ctctctctct gtggtctaca ttatagctgg |
| 67261 | agtacaattt ccaggcctca tttccagcat tctcttctgg aactttcatt tggctgccac |
| 67321 | acagaaggcc ctatgtttcc tgccagctgt cctctgggtt ctcaagtacc ccaggttcac |
| 67381 | ctactagaga aaagaattcc aaaatgccag ctgcccaagc agctctacct gcctctctgc |
| 67441 | cctgattccc agagtaaggt ttcagccatt aagaactgtc tctgggcatc tctgaaacac |
| 67501 | agccttcaac acaatggtct atctggttga tcaccactgg ccatgaagct tggcctcaac |
| 67561 | gcctgtccca tcttagggga tggttctaat ctcaggtcta gctccttcct tttagtcaag |
| 67621 | aattgaggaa tcatgaagtg ttctctgact ccatatgttt gtactaggct tcttcatgat |
| 67681 | gatttatttg agttttgatg tacttggttt tggctgaact cacatctccc tctcctgctc |
| 67741 | cctctcccta tgcccttgca aggaacaaga actacaaaat ggagcctttt gtaaggccgt |
| 67801 | tgcactcagg agctttcttg aagtgctctg aaatttctgg cagaaataaa gctatttaaa |
| 67861 | aaaaatagtt acagcttatg ttcctcttct tctgtgccct gagaaatagt taatcaactc |
| 67921 | ttccttagct tggagttcac ccagatgctc cttaaggctt atctgtagct ccactctcag |
| 67981 | tggtaaagcc accactggat cctagcaggg tgcctaaact cagtaatgtg gtaagagata |
| 68041 | aggcaacaaa gcttcatgaa cattgtattt tcttccaaca gggcacaagg aagatattaa |
| 68101 | actgagttcc ccccaaaatc ccaaaaatct cctactgata atcaagattt gtggaatcta |
| 68161 | taatgtgtct ttattttggt ttaaactaat gacactaKat tttcttctac ctggcaaaga |
| 68221 | agaggtaggt atacattaaa caaaagaaaa tgaaaaaaat ggacctaaaa tcatgtggga |
| 68281 | aatagatcat tgtcatcatt gttcaaaaaa gaaatgttgt ggaatatttc tgcaatttcc |
| 68341 | tggccagcta gtctttttca ccagacgcat atatcttttt caaatatcaa aagatagtaa |
| 68401 | agctatgcat gtaaaagtca taatagtaat agtaattata agtattataa gggtaagaat |
| 68461 | aatttacatg attcttactg actcccaagc atttgctaag cactttaact tttcaaaatc |
| 68521 | tcagaatgct atgctgtaca tatgataaga aaggagaact ttcttagttt agaaaactga |
| 68581 | gttgcagaaa ggttaagcaa cttgatcaaa gcaaattagt aatgggaaga gcagaaattt |
| 68641 | aaacctaact catctttcat acctatgaag atgcaaataa gtcaaccaat tacatttgtg |
| 68701 | tgtaatatat ttgccctctt gatgaataYt gaaccagagt tagagcatct tctatttgtt |
| 68761 | tcatgaaaaa gcattgacat tatacacgat ttgtgtgttc tactttgaaa tcacactttg |
| 68821 | ttactctaaa cattatttga ataagaagtt gctaataatt tgagcagtta tttcaaaata |
| 68881 | tttatgtact attactacac agatggatta ctcttgagct tggcatccaa tttttaaaat |
| 68941 | tttctaactc atgtgtacat gactaatttt ccctctcaga attatgacaa cattcattca |
| 69001 | tatgctcttc tctcttgatt tttgtacttt ggtagtttgg gaaacatgat aagatagMtc |
| 69061 | gtagtataac taatagctta gcacacatag catatctata ctcttttgac ttttcctcta |
| 69121 | aatatcacct tatcatagtt tgttctttat cccctatcca cttgccatta tttccatcca |
| 69181 | atgaaactta tttttcttct ctctcatctt tgaatttcct tccctcccta acagcatgtg |
| 69241 | agttacttga aagctctttg atcagaataa aaatcccaat tatttgatac ttccatttat |
| 69301 | ttggattata aagacatacg ggggtatatt ttgcaaaaca gtcttgtcag ccccggcaga |
| 69361 | agatatatca ctatattata gctttgccat gtgtccatag agaaaggtta tcttatgtta |
| 69421 | aacacatgtt catatgcaat aacagagaca tgaattctaa ggatattgca gaaagcatag |
| 69481 | ctgactactg attctagcac cttcaggatc tttctcttct tagttgYgac cactgtaatg |
| 69541 | cacttcatag agtaataaat tgttcttgcc ctgggatcat gattcccatc tcttcactac |
| 69601 | aacatgtcta gatgtaagac atgtttaccc ttgtataatc aatgatctga gtctagatgt |
| 69661 | caagtacttg cttgttctgt gcattaactg tcttcttttt ctggaactgc aggctctatc |
| 69721 | agatccttcc cctaaaattc atacatcctg ccttgttact atccgtgccc cagcatgacc |
| 69781 | aatatcaaag tggttaagaa aaaggctcca gctattttag tatcctaaat tttatctttt |
| 69841 | cattctacaa agaagttttt cataaaaagt taaaataaat ttaaaaactc attgatacta |
| 69901 | ttcagtaaga tgttatagat tccaagcaac tatctctttt ataggcaaca taattaaaag |
| 69961 | tgaattacaa atgcagactt tatgactcta attaaactct aggccacaat gcattaaaaa |
| 70021 | cagattatac atattaagtt agatataaga aaaaataaat atgccacata gtgaggcata |
| 70081 | agttaatatg cctcaaaata aatgaggaga aacaatttca gataatacat atttatataa |
| 70141 | aatgtttatc aacataacct aaaatatttt cttgaatttc atcagagata aaatttttat |
| 70201 | acttaatttt tgtaaacata tacatcatac tttctctcac ttacttcact acttcattcc |
| 70261 | atcccaagaa catctgtgca agtatttccc aagtgtggga gactggaaga ggttctatat |
| 70321 | tttggttgta agaatatata ttaattttta gaacattata attaaactca gaaatattta |
| 70381 | attgtaggga tattatttgg taagaatcaa ataaaatgac taaaaattaa cttgaggcaa |
| 70441 | catgaaatct caacatcatt gtaaaactaa gagagcagat cttaaacaaa atgtagtgca |
| 70501 | gtcttcacac ttgggagatt cattaatcac ttaagtgttt tctctgaagt ttttaaatgc |
| 70561 | ttaaagattg ggttacatta tgaaaaggac ctgtttaata ttgtgtctgt aaagttcaat |
| 70621 | tctacctagt tctcactttc tttcgtatct tctctagtga agaatgaaga aatgagaatt |
| 70681 | atgtgtttga atctagcacc tctgacatgg aaaaacacat ggtgaagcag aggaaagaaa |
| 70741 | actgaaagaa agtgaaaata tctttaagaa tacYttgggg aaacaaacag tatttcaaat |
| 70801 | gaaagattaa gaacaaatcc aaacaaatgg attggaaggt attatgtaaa aaaatatgag |
| 70861 | atgctttagc acaggtactc taagaatttg agaagcttct catgtttaag ggagaaggta |
| 70921 | acattcctga tagaaacatt taaaggcagt gcagaaagga aattctcatc aggaggccta |
| 70981 | tagaacacac atgggcaaca gcaagccccc cttgccctgc tcagccctgg atccatacag |
| 71041 | aggaaaggag agaggtaaag atgaaagaaa gaggttgaac agaagatagt aaatggttga |
| 71101 | aaaatatttt taaaatagaa agagttatgc ttctgagaag attctccatg tctgggtatc |
| 71161 | agacatggag atacatcata ctgatcaacc tgaagtaatg tacttactga aatacccact |
| 71221 | tctagttcat cWagttcatg caaggtttca caaagccacc aatcgtttta atcatcttat |
| 71281 | aaaagctttc taattgaaat attcttaagt atcacatttt actagtggca ggtatctttt |
| 71341 | caaaaaaaaa atacaagcta agcaacttgt cctctgttgt ttcctaccac taagtttgca |
| 71401 | ttagaagaga ctggcagtca aacacacaag aagaaacaca agtattggca atttaacaga |
| 71461 | aacaaatggt agtgtatcat ggacatctaa ggtttgtagg actaaatcat accctcttct |
| 71521 | agtaagtcct gaaaaaaatg tatttaaaac atatgttgaa gaaaatagct tttagagggg |
| 71581 | cagataatag gctttggact tagaaatttt ataaaaatcc attgtgcatt ctttaggctt |
| 71641 | ctagtcctat tcagtttttg ctatgagcta tgatttgttt gtggaagcaa agtaaaggca |
| 71701 | gcctatttct taccgcctcc acttcggctg ggtcatacca gacgttgatg tagggatgct |
| 71761 | gtaaggcgtc gtccactgat attctttttg ctgggtcaat cactagcatc tttgacaaca |
| 71821 | agtccctggc ttggctggct gaaacaataa atgagaaaaa caattagtaa gattttgatt |
| 71881 | tcagtaagga aatttgtcag agaggaaatt taagaaaaca aagtatggaa tagtatcttc |
| 71941 | ctKccaaaca tcaggtaaat cccttgatgt gatggaaaag aaaatgcatt ttacacctgt |
| 72001 | ttctttgtgt ctctaagcct aaggcttcat tctgaggctc tccaagggtt gtaacacttt |
| 72061 | gggtatataa ttattcacgc aaacttctta gtaaaatgta tcaacctgga gagtttatgt |
| 72121 | aagcaaagtc atcttctttg gtaacctgca tgaatatagg ataaacagaa taaagagcac |
| 72181 | agagtgcaaa tgcatgatgt tcaacaaatg tgtggacggc tgtttgaagt taatcaacac |
| 72241 | gtgtgtctct atattcttat ttatacaacg gaattatcag aattttagta tataaattga |
| 72301 | cttaagagtt tgtatcatac aaatttttct tttctttttg tttttgttta tttatttatt |
| 72361 | tatttatttt tgagacaggg tatccctctg ttgccaggct ggagtgtagt ggaatgatca |
| 72421 | cagctcactg cagcctggaa ctcctgcgct caaaggatcc tcccatccca gcttcccgag |
| 72481 | taactggaac tacaggcatg caccaccatg cctggctatt ttttctttct ttcttttttt |
| 72541 | ttgtaaagat aagatctccc tacattgccc aagctggtct cgaactctgg gctcaaagat |
| 72601 | cctcccacct cagcctccca aaatgctggg attaaaggag tgagccactg ctcccagcca |
| 72661 | caaatttctt atatacataa aaacaatcaa agaattgaag tgatagaaat aatatcattc |
| 72721 | aactactgaa ttttatttta acattctgta atcatcaggt atttttaatc aatggaatca |
| 72781 | tgacttgatg aaatccagta gttaaagact gaagccagtc taatgctaca ccagacacta |
| 72841 | atgagaaact ctatattatg taaaggttat cataattaat tttatggttg tgggcagtaa |
| 72901 | cagttataaa attatatggt gatagctatt tatttagtaa gattgttaaa agacacatta |
| 72961 | attttaacct ctaaaaatat gtaaattttt taaattatta tactttaagt tctgggatac |
| 73021 | atgtgcacaa catgcaggtt tgttacatag gtatacacat gccatagtgg tttgctgcac |
| 73081 | ccaccaacct gtcatctaca ttaggtattt ctcctaaagc tatccctccc ctagtctttt |
| 73141 | acataaagtt ttaacagaaa tgaatatctc aaatcaatat tgttactcaa ctgatctgaa |
| 73201 | agttgaaccg tgaaaatatt cagtttattc taactatatg gtaaaacatc aaattaaaga |
| 73261 | taacaaaatt taaatggatt tttcaaaaaa tcaacataga tgactctgga tacatatcaa |
| 73321 | tttttattta atcaattttt cagattaaaa caacttggct aattctacaa atttgaaaat |
| 73381 | tatataaaat acaaaaWtca ggaaataata tctaacccca ctagctagag gcaatgactg |
| 73441 | tgattattgg catatttttt ctagtgccgt tcatgtattt ttcacgtaat attaaccata |
| 73501 | ctatagttaa aatgtagcaa ttaatttttt acctgtaagt attttcttgg taattacatc |
| 73561 | aataaatata tcttttaatt ttctgaacaa atatgaaaaa tcactagtta aacttgagaa |
| 73621 | ttttttattt ctattaatca tttttctggg aaactcaatc ctagaaactg attatcatta |
| 73681 | ctttcaaatt aaaactctgt accgcacagg aataacaaaa atacagaaat acttatacat |
| 73741 | taattcaata tttaattttt ctcattgcca atggcctcct aatagcctaa cagggctgtg |
| 73801 | tttatttatt cattgcttta aagacctctt taatttgtaa ctgattttac atccaaattc |
| 73861 | atgtaccatt ctcataagat tctctcatca aatctatcca tttttctgct cttagaaagg |
| 73921 | gctggggtta gccgggcgtg gtcgtggctg acacctgcaa tcccagcact ttggaaggcc |
| 73981 | gaggcaggca gaccacgaag tcaggaggtc gagacgatcc tggctaccac ggtgaaaccc |
| 74041 | catctctact aaaaatacaa aaattagctg ggcgtggtgg cgggcgcctg tagtcccagc |
| 74101 | tactcgggag gttgaggcag gagaatggag tgaacctggg agacggagct tgcagtgagc |
| 74161 | ctagatcggt atcccgggcg acagagcgag actctgtctc aaaaaaaaaa aaaaaaaaaa |
| 74221 | aaagaaaggg ctggggttac aatggattct tcatgaatag ggtcatgaac tacaccaata |
| 74281 | caaaatcatc ttgctaagaa acaatactat ttttaggaaa caatatggga aaatgtttca |
| 74341 | atgctttatc tttcattctt tcatttgaaa gcaaatgtgt tatgctcttt tccaaacaca |
| 74401 | gtagaataca ttcttctctt tggttatttg tatattaaga ttatttataa tttgccaatt |
| 74461 | ttatattaaa aattaaattc aataatcaaa tatgcaaact agtttatcat ttacaaacat |
| 74521 | aattgcagag attcaaattg atttaaaaag taaactcaag aaaattggaa ttacattatg |
| 74581 | aagtataata tatatctatt ttttgagacg gagtcttgct cactctatct ctaggctgga |
| 74641 | atgcagttgc ccgatctcgg ctcactgcaa cctcttcctc ctaggttcaa gcgactctcc |
| 74701 | tgcctcagcc tcctgagtag ctgggactag aggtgtgtgc caccatgccc agctaatttt |
| 74761 | tttatatttt taggagagac gcagtttcac catgttggcc aggatggtct gtctcgattt |
| 74821 | cctgacctct tgatcctccc gcctccgcct tccaaagtgc tgaggttaca gacttgagcc |
| 74881 | accgcccccg gccctgaata tccagacaat tctaaaatcc gtggctactc ccctttgtag |
| 74941 | gaacgatctt tgaagaaata tttctttaac ttgaggctgt gccaattaaa gaattgatca |
| 75001 | atgttttagg tgatgggtta tgctaattgc tctgatttga ccattatacc tctatacatg |
| 75061 | tttcaaaaat accaccctat atcccacaaa aatgtacgat tatcatgtgt caactaaaaa |
| 75121 | taaaagggaa caaagagaaa gagatagagg tctctatact gtgtgcttgt ttggcccgtc |
| 75181 | accctcatag ttgtcctcaa gtcattcctg aagggcatac ctttgagttt attgtgctcg |
| 75241 | gagtccgctg ggaagaggga atctgggaag agtttgggga aggtgagtcc cgcatacttg |
| 75301 | ggccgattct ccacatagtt tcttactgtg ggttgcaatt tcttcatgaa ttctggacat |
| 75361 | ggtgttccta gttgttcaat taccttattc cactggtcaa tatctgagaa ttgaacagtt |
| 75421 | aagggaaaaa agaagagcag accactagcc tttcaacaat tgggagacta actcttctca |
| 75481 | aagtcctcaa cagtcattaa tgcaactgta gttttaataa gcaacaaaga atgaaaatat |
| 75541 | aaaatcagac ttgcaggcta tgagtcttct tgaaatttta aattcacttt tgaaacttat |
| 75601 | atacttttgg aaaactagca attatttaac aaaataagat atcatgaaat agtctttggg |
| 75661 | gtgtacagat aaaaagtcat tcagataaca caagatgaat ctcatattct gcagataaac |
| 75721 | ttctatataa atcatgtgat tcagaagaaa aatatataat gacacatcat agtgacattt |
| 75781 | ttatcctgaa gttacctcaa ttatttatat ttttaaaatg acaaatacac aaatatttta |
| 75841 | gattatattc atttacattt tcctaaacta tctttaagat attttttctt ctctaaaaat |
| 75901 | ctggaatttt tttctatttt tatgcatatc aacataactt ttatagtaaa acaactcatt |
| 75961 | agttctttgt cagattccta aaaatcatat ttattaaaca aagaagccac ctatttcata |
| 76021 | cctattgttt tactgatgat atcactgaat attgttttca atgaaatata agggtgaaca |
| 76081 | gaggaatatt caatttaaaa caacagattt ctatccaagt acatccaagt ttagaaagta |
| 76141 | cattaagcat ggggaatacc acagaaaaaa tattacctct ctatgaaaat taataccctt |
| 76201 | ttaaattgga gctagctctt tttgcagaca tactcaatga ttgagattct cttttttatc |
| 76261 | tgtcattaat attcgatata tcagagcctt gataatgata aacaccatct actctgaaga |
| 76321 | aYcaactgga aacaactgat ttaaacagat ttcctcgcca tgtgccagtg aataatagag |
| 76381 | atcttggaaa ttcccttaaa tccaatactg caagcagtat tctctcctac attgcttatt |
| 76441 | cttctacaac tgttctttgg tgttaagtta gttaactcat ttccactaag gtaaggtgtg |
| 76501 | acagttaagg gactcttacc ataatattac ttagttgaag tttgtcaagt ctttactcct |
| 76561 | agctattcaa gaggtttgga ctgaaaaaga ttgtcagtcc tgattaaaga taaacttaac |
| 76621 | tgtattcaac aaaaagaaat atataataca aacagtagat aggagattga acaaagataa |
| 76681 | aatcaaacta atttgcaaat ccttttcttc atctctataa tctggctgaa taaaacttaa |
| 76741 | attattacat attctgtcag ttattggaat aaaaaatata taaccatgga ctatgaagat |
| 76801 | atgaaatgaa ggtgggaaaa acaataatat tccttaataa agcagtagta ataattaaac |
| 76861 | aagaacatgc tttgatagaa gctcatctaa ttatcatgtg catgagaaaa tacagcagta |
| 76921 | attcagaatc actagaaata atgaaactaa aacattatat aaggttagag ttacaaatta |
| 76981 | ctcttagaca tgagcctgta ttaatactat ggatataggt aagctataag tacttagaca |
| 77041 | tacttaaata gaatacatta gtgtagttaa ataaaaccta agtgcttgtc attataaatt |
| 77101 | atgaagagac attttttaaa cagttaggtt agcatgccct accaagctat catttcatca |
| 77161 | ttgatcaaac ttcctcaaaa ttctaaccac tcgatgtcaa acttaaaata gaggcaaaga |
| 77221 | agatgtcaga ttttctgtaa ataagtcatt taacaaagtt ggcaaaataa tcccaacaca |
| 77281 | gtattatatc ctaattacat ttgctataat tataaagata tacagtaaat aattgatttt |
| 77341 | aaagaagtac tcccaaaaca aaaaaaactg atacttttat attaaagttt aaggagagtc |
| 77401 | agatattctg gaatttgtta aataagaatt gttaaaatgt ttcttgtaat aaatcataat |
| 77461 | tagaatctag agacacactc agcgctcttt cctaccttag gttgctttta aatgaaacac |
| 77521 | tgttgtgatt cttgatgcaa tggtttacaa aaagaagggt ggcaaaaatc tttatcattg |
| 77581 | tcaatgaaat agttaaatgt tcatactgca aatctgagtt acagtgatct ttaaaagtct |
| 77641 | gtgtctttat tttaaccaat ccatttcaag atttttcttc ccaaaaacat atgtgtgtgt |
| 77701 | atatctacat atggtaagta cttgttttct agatgttcat agaatacaaa catgaacaac |
| 77761 | tatgtccaat ttagttgagc acatgatttt tctttctttt tttttttgta gcacttgaag |
| 77821 | ggctttattt ttttttcttt ttcttttttt tattatactt taagttttag ggtacatgtg |
| 77881 | cacattgtgc aggttagtta catatgtata catgtgccat gctggtgcgc tgcacccact |
| 77941 | aactcgtcat ctagcattag gtatatctcc caatgctatc cttcccccct ccccccaccc |
| 78001 | caccacagtc cccagagtgt catattcccc ttcctgtgtc catgtgatct cattgttcaa |
| 78061 | ttcccaccta tgagtgagaa tatgcggtgt ttggtttttt gttcttgcaa tagtttactg |
| 78121 | agaatgatga tttccagttt catccatgtc cctacaaagg acatgaactc atcatttttt |
| 78181 | atggctgcat agtattccat ggtgtatatg tgccacattt tcttaatcca gtctatcatt |
| 78241 | gttggacatt tgggttggtt ccaagtcttt gctattgtga ataatgccat aataaaaata |
| 78301 | cgtgtgcatg tgtctttata gcagcatgat ttatagtcat ttgggtatat acccagtaat |
| 78361 | gggatggctg ggtcaaatgg tatttctagt tctagatccc tgaggaatcg ccacactgac |
| 78421 | ttccacaatg gttgaaccag tttacagtcc caccaacagt gtaaaagtgt tgctatttct |
| 78481 | ccacatcctc tccagcacct gttgtttcct gactttttaa tgattgccat tctaactggt |
| 78541 | gtgagatggt atctcatagt ggttttgatt tgcatttctc tgatggccag tgatgatgag |
| 78601 | cattttttca tgtgtttttt ggctgcataa atgtcttctt ttgagaagtc tctgttgatg |
| 78661 | accttcgccc actttttaat ggggttgttt gtttttttct tgtaaatttg tttgagttca |
| 78721 | ttgtagattc tggatattag ccctttgtca gatgagtagg ttgcgaacat tttctcccat |
| 78781 | tttgtaggtt gcctgttcac tctgatggta gtttcttttg ctgtgcagaa gctctttagt |
| 78841 | ttaattagat cccatttgtc aattttgtct tttgttgccg ttgcttttgg tgttttggac |
| 78901 | atgaagtcct tgcccatgcc tatgtcctga atggtaatgc ctaggttttc ttctagggtt |
| 78961 | tttatggttt taggtctaac gtttaagtct ttaatccatc ttgaattgat ttttgtataa |
| 79021 | ggtgtaagga agggatccag tttcagcttc ctacatatgg ctagccagtt ttcccagcac |
| 79081 | catttattaa atagggaatc ctttccccat tgttgttttt ctcaggtttg tcaaagatca |
| 79141 | gatagttgta ggtatgcggc gttatttctg agggctctgt tctgttccat tgatctatat |
| 79201 | ctctgtttag gtaccagtac catgctgttt tggttactgt agccttgtag tatagtttga |
| 79261 | agtcaggtag tgtgattcct ccagctttgt tcttttggct taggattgac ttggcaatgc |
| 79321 | gggctctttt ttggttccat atgaacttta aagtagtttt ttccaattct gtgaagaaag |
| 79381 | gcattggtag cttgatgggg atggcattga atctgtaaat taccttgggc agtatggcca |
| 79441 | ttttcacgat attgattctt cctacccatg agcatggaat gttcttccat ttgtttgtat |
| 79501 | cctcttttat ttccttgagc agtggtttgt agttctcctt gaagaggtcc ttcacatccc |
| 79561 | ttgtaagttg gattcctagg tcttttattc tctttgaagc aattgtgaat gggagttcac |
| 79621 | tcttgatttg gctctctgtt tgtctgttgt tggtgtataa gaatgcttgt gatttttgta |
| 79681 | cattgatttt gtatcctgag actttgctga agttgcttat cagcttaagg atattttggg |
| 79741 | ctgagacgat ggggttttct agatatacaa tcatgtcgtc tccaaacagg gacaatttga |
| 79801 | cttcctctgt tcctaattga ataccgttta tttccttctc ctgtctaatt gccctggcca |
| 79861 | gaacttccaa cactatgttg aataggagtg gtgagagagg gcatccctgt cttgtgccag |
| 79921 | ttttcaaagg gaatgcttcc agtttttgcc cattcagtat gatattggct gtgggtttgt |
| 79981 | catagatagc tcttattatt ttgaaatacg tcccatcaat acctaattta ttgagagttt |
| 80041 | ttagcatgaa gggttgttga attttgtcaa aggcttttcc tgcatctatt gggataatca |
| 80101 | tgtggttttt gtctttggct ctgtttatat gctggattac atttattgat ttgcgtatat |
| 80161 | tgaaccagcc ttgcatccca gggatgaagc ccacttgatc atggtggata agctttttga |
| 80221 | tgtgctgctg gattcggttt gccagtattt tattgaggat ttttgcatcg atgttcatcg |
| 80281 | aggatattgg tctaaaattc tcttttttgg ttgtgtctct gcccggcttt ggtatcagaa |
| 80341 | tgatgctggc ctcataaaat gagttaggga ggattccctc tttttctatt gattggaata |
| 80401 | gtttcagaag gaatggtacc agttcctcct tgtacctctg gtagaattcg gctgtgaatc |
| 80461 | catctggtcc ttgactcttt ttggttggta aactattgat tattgccaca atttcagctc |
| 80521 | ctgttattgg tctattcaga gattcaactt cttcctggtt tagtcttggg agagtgtatg |
| 80581 | tgtcgaggaa tttatccatt tcttctagat tttctagttt atttgcgtag aggtgtttgt |
| 80641 | agtattctct gatggtagtt tgtatttctg tgggatcggt ggtgatatcc cctttatcat |
| 80701 | tttttattgt gtctatttgg ttcttctctc tttttttctt tattagtctt gctagcggtc |
| 80761 | tatcaatttt gttgatcctt tcaaaaaacc agctcctgga ttcactgatc ttttgaaggg |
| 80821 | ttttttgtgt ctctatttcc ttcagttctg ctctgatttt tgttatttct tgccttctgc |
| 80881 | tagcttttga atgtgtttgc tcttgctttt ctagttcttt taattgtgat gttagggtgt |
| 80941 | caattttgga tctttcctgc tttctcttgt gggcatttag tgctataaat ttccctctac |
| 81001 | acactgcttt gaatgcatcc cagagattct ggtatgttgt gtctttgttc tcgttggttt |
| 81061 | caaagaacat ctttatttct gccttcattt cgttatgtac ccagtagtca ttcaggagca |
| 81121 | ggttgttcag tttccatgta gttgagtggt tttgagtggg attcttaatc ctgagttcta |
| 81181 | gtttgattgc actgtggtct gagagatagt ttgttataat ttctgttctt ttacatttgc |
| 81241 | tgaggagagc tttacttcca actatgtggt caattttgga ataggtgtgg tgtggtgctg |
| 81301 | aaaaaaatgt atattctgtt gatttggggt ggtgagttct gtagatgtct attaggtccg |
| 81361 | cttggtgcag agctgagttc aattcctggg tatccttgtt gactttctgt ctcgttgatc |
| 81421 | tgtctaatgt tgacagtggg gtgttaaagt ctcccattat taatgtgtgg gagtctaagt |
| 81481 | ctctttgtag gtcactcagg acttgcttta tgaatctggg tgctcctgta ttgggtgcat |
| 81541 | atatatttag gatagttagc tcttcttgtt gaattgatcc ctttaccatt atgtaatgac |
| 81601 | cttctttgtc tcttttgatc tttgttggct taaagtctgt tttatcagag actaggattg |
| 81661 | caacccctgc cttttttgtt ttccattggc ttggtagatc ttcctccatc cttttatttt |
| 81721 | gagcctatgt gtgtctctgc acgtgagatg ggtttcctga atacagcaca ctgatggatc |
| 81781 | ttgactcttt atccaatttg ccagtctgtg tcttttaatt ggagcattta gtccatttac |
| 81841 | atttaaagtt aatattgtta tgtgtgaatt tgattctgtc atcatgatgt tagctggtga |
| 81901 | ttttgctcgt tagttgatgc agtttcttcc tagtctcgat ggtctttaca ttttggcatg |
| 81961 | attttgcagt ggctggtact ggttgttcct ttccatgttt agcgcttcct tcaggagctc |
| 82021 | ttttagggca ggcctggtgg tgacaaaatc tgtcagcatt tgcttgtctg taaagtattt |
| 82081 | tatttctcct tcacttatga agctcagttt ggcctgatat gaaattctgg gttgaaaatt |
| 82141 | cttttcttta agaatgttga atattagccc ccactctctt ctggcttgta gggtttctgc |
| 82201 | cgagagatcc gctgttagtc tgatgggctt ccctttgagg gtaacccgac ctttctctct |
| 82261 | ggctgccctt aacatttttt ccttcatttc aactttggtg aatctgacaa ttatgtgtct |
| 82321 | tggagttgct cttctcgagg agtatctttg tggcgttctc tgtatttcct gaatctgaac |
| 82381 | gttggcctgc cttgctagat tggggaagtt ctcctggata atatcctgca gagtgttttc |
| 82441 | caacttggtt ccattctcct catcactttc aggtacacca atcagaccta gatttggtct |
| 82501 | tttcatatag tcccatattt cttggaggct ttgctcattt ctttttattc ttttttctct |
| 82561 | aaacttccct tctcgcttca tttcattcat ttcatcttcc atcactgata ccctttcttc |
| 82621 | cagttgatcg catcggctcc tgaggcttct gcattcctca catagttctc gagccttggt |
| 82681 | tttcagctcc atcagctcct ttaagcactt ctctgtattg gttattctag ttatacattc |
| 82741 | ttctaaattt ttttcaaagt ttttaacttc tttgcctttg gtttgaatgt cctcccgtag |
| 82801 | ctcagagtaa tttgatcatc tgaagccttc ttctctcagc tcgtcaaagt cattctccat |
| 82861 | ccagctttgt tctgttgctg gtgaggaact gtgttccttt ggaggaggag agatgctctg |
| 82921 | cgttttagag tttccagttt ttctgttctg ttttttcccc atctttgtgg ttttatctac |
| 82981 | ttttggtctt tgatgatagt gatgtacaga tgggttttca gtgtggatgt cctttctgtt |
| 83041 | tgttagtttt ccttctaaca gacaggaccc tcagctgcag gtctgttgga ataccctgct |
| 83101 | gtgtgaggtg gcagtgtgcc cctgctgggg gatgcctccc agttagactt ctcgggggtc |
| 83161 | aggggtcagg gacccacttg aggaggcagt ctgcccattc tcagatctcc agctgcgtgc |
| 83221 | tgggagaacc actgctctct tcaaagctgt cagacagggg catttaagtc tgcagaggtt |
| 83281 | actgctgtct ttttgtttgt ctgtgccctg cccccagagg tggggcctac agtggcaggc |
| 83341 | aggcctcctt gagctgtggt gggctccacc cagttcgagt ttccaggctg ctttgtttac |
| 83401 | ctaagcaagc ctgggcaatg gcgggcgccc ctcccccagc cctcctgggg ccttgcagtt |
| 83461 | tgatctcaga ctgctgtgct agcaatcagc gagattccgt gggcgtagga ccctccgagc |
| 83521 | caggtgtggg atatagtctc gtggtgcgct gctttttaag ccggtctgaa aagtgcaata |
| 83581 | ttcgggtggg agtgacccga ttttccaggt gcgtctgtca cccgtttctt tgactcggaa |
| 83641 | agggaactcc ctgacccctt gtgcttccca ggtgaggcaa tgcctcgccc tgcttcggct |
| 83701 | cgcgcacggt gcgcgcaccc actggcctgc gcccactgtc tggcactccc tagtgagatg |
| 83761 | cacccggtac ctcagatgga aatgcagaaa tcacccatct tctgcgtcgc tcacgctggg |
| 83821 | agctgtagac cggagctgtt cctattcagc catcttggct cctccccatg atttttcttt |
| 83881 | ataataacaa ttaacctaac ataaacgaag actggaactc ctatctcctg tttccatgtt |
| 83941 | cagtgcttgt ttcaagtatt ccagtaagaa aaagtaaatt tggaattgat atggtaaaaa |
| 84001 | attgttttaa tttggccata aagttaatgg tgtgttttgt cacaagcaac tcccttaaag |
| 84061 | aatgtttctg aagccccttc cttagtctat caggccaaca tattcctgta aaacaattaa |
| 84121 | ttttatcaat attttatata gaaaataagg tgatttttaa atgggaaaac agatttccac |
| 84181 | aaggattaca tttttaaagt acataaaata ttaactagat tttatgatta tgtattaatt |
| 84241 | gttaacatgg taaatatgtt tttaaagaat gaccaaaacc attttttgta gaagaattta |
| 84301 | tttgctttag aatacccata aattaccatt tttataccac atagagtaag gatttacaga |
| 84361 | tcttagccct gtgaggttat cagaaaagca ataagtatat tttttatatt tttgatgtgc |
| 84421 | tgtttcagaa attatcaccc ttaacataaa ttatgaaatg cattgaatac aaatggaaaa |
| 84481 | atggcattta ataagaatta tttgacagta aatcacctga tataattcta aattgactac |
| 84541 | ataaaagtag agttatattg taagaagttc aattttataa gtagggcatg atgtccagat |
| 84601 | aatgtacaaa tgtgatatta ctaaggtatt caataaatag tattataata gaataaaaca |
| 84661 | aagtaaatat gaatagtaaa aatgagtatc tgaaatccta agattgaaaa tttttccaat |
| 84721 | taatattatc aattagctaa taacttatat ttcaagcact tctcttactt tagcttcctt |
| 84781 | tgttagggaa ctttctaaaa cgataatgcc ttgaacataa tatatgaaaa gggcaatttt |
| 84841 | tgggcctctc atttgcacag gccccttata agactaatcc ttttctgcgt attgtgggtc |
| 84901 | aaaattatga acagaattta tgccatccat atttaagcct tcagaactta ctaagtggaa |
| 84961 | tcataaggaa ctatttaatg gcctcatcca attgtttcct ttgaatgact atatcttctg |
| 85021 | tttatgtttt cttctatcat tgattgcttt ttgttactat gaatgtgatt tccagagcca |
| 85081 | acacctcata gtttctgact tgctttgtac tctgctctag gccttatatc caatactttg |
| 85141 | tgcaaaaatt ttctgacctc tctttgcaat ttaaagaaca tcagtgttta tctctaaatt |
| 85201 | attagctatg ttcttaaaga caatattgcc taatagcaat gtcgttatag aatagttcca |
| 85261 | ggagaggctt ttatcatgca cgccaaacag ctgtttaggc acaatgttga gtaggcttgt |
| 85321 | gtggctttat attagcttcg taggaaagaa ttttgtgatc agttagtaat gtctgcaaca |
| 85381 | gatgcacaat aaggaaattg aagcacatat gccatgtgtt aagcatcatt atattaaact |
| 85441 | gtggttccca aacttgaata ttcgtaacgc agggaactta caaaaataca tatccctcct |
| 85501 | ccctactcat tgatctacaa gattctgagt ccacaggtct gggctgatat cataaaaaac |
| 85561 | accagtggta attgtaatca aggatgggat tatattttga gaaatactaa tgtagaatat |
| 85621 | actaaaaagt ctcttgtgag ttactaagtc caaatgtaag agtaaaatac taacatgttt |
| 85681 | cagttttatg acacaaatta gaataggatc aaattcacca aaagattatt ccacacctgt |
| 85741 | ttgttaactt gggggtcatt cacctgagaa tactcagaga aagaaaagtg ctcatcttta |
| 85801 | tttctttgcc ccatgtaatt ttcctgcaaa ggccatggga aattacagta agtggcacat |
| 85861 | gcagtctcct agtagaggga gaagaattct gcccctcatc atctgtggga gaactgaaat |
| 85921 | tgtatgctgc tattacctgg ggagtagtac ccatggaaag tactaattat agttctttgt |
| 85981 | gattaaattt gttgttaaaa gaattttttt ctaaaaacaa gtataaggaa atacatatgt |
| 86041 | atatatatat atacacacac acacacacac acacacaaac atatgataga gttctgaatc |
| 86101 | tagattttca tggttatgtt tctaattgtt ttgtttcttt aaaaatgttt ttaggggcca |
| 86161 | gggacagagg ctcatgcatg taatcctagc actttgggag gccaaggcgg gaggatcacc |
| 86221 | tgagcctggg agttcaagac cagcctgacc aacatagaga aaccccgtct ctactaaaaa |
| 86281 | tacaaaagca gctgggtgtg gtggcacatg cctgtaatcc cagctactca ggaggctgag |
| 86341 | gcaagagaat cacctgaacc catgaggcac aggttgcagt aagcagaggt agcaccattg |
| 86401 | cactccagcc tgggcaacga gagtgaaact ttgtccccca cccccccaaa aaaagaaaaa |
| 86461 | gaaaaatgtt tttagggtat tttttatttg aaaatgttct tttcctctca aaagtccttg |
| 86521 | tcagattctg gtaacaggaa atatgaataa gtgacttaat aaacaattgt aactaacatc |
| 86581 | tgttttaatt ataaatgagc ataggaagag gaattataag ccaaaacaca tccaaaatat |
| 86641 | gaagctggca aattaagtaa aaggctattt gtacactgac actagcacat tgctagtgtt |
| 86701 | ataataaggg acatggaata tccaaaggct gtttgaagca tgctataaat ctattcacta |
| 86761 | ttataatcta tctttactta caaataaaaa aatggagaga catgtaacat atatattaat |
| 86821 | atgtgtagca atggcagaat tttcattaag ctgttgaagg ttaatctttg gggtgttcca |
| 86881 | tttgcacagg cctcttacaa gactaatttt ttactggtaa ttttattttt tgtaaatagc |
| 86941 | acccaccttc agttgtatga tattctgacc tcaaaaatgc tggatttgat tctctcagcc |
| 87001 | tgtgatagta ttttgaaaat gagaagaggc cttggagatc atgtaatttg gcatctctat |
| 87061 | tttaaagctg aaaaatatta aagtcctttt caatttggta aataattata aacaaagaag |
| 87121 | ctgatacaaa cagcctacac gaacagaaat atagaatcta cttaagagaa atttaaggac |
| 87181 | actgtactct gttctgtaag accaaatcgg gaaagatact ttcacttctg tatataacac |
| 87241 | tacatacaat ggaagttaag aagaggcaat caagcttata tgaggtaaga aaacctgcca |
| 87301 | cttaaagcat agctgaaaaa acttttgata tttaaattta aaaaggaaga tagaagtaat |
| 87361 | ccctgccccc atgctagcat tttggtcttt tggtctctct tactctgcag ctattatcac |
| 87421 | cttcttctag tattctgcac cttttaatgt aagtatcatg gtaataagaa tttttatctg |
| 87481 | ttttgtttac taatatatct ctggagacaa aaagtaccca gtacatagta ggtactcaaa |
| 87541 | aatatttatg ttataaatga atgtctattt tatgtccaga gttttgtaca cacataatat |
| 87601 | aaagaatgaa aaaaataaaa tattcgaaga aacgacagct gagaaattcc agtaattgaa |
| 87661 | taaaggttgg aatctaggaa acaatgactc tcaaggaaga taaataaaaa gaaatccatg |
| 87721 | cctagtctcg tagtagagaa acagcagata atcaaagacc aaaaaaaaaa atcccaaatg |
| 87781 | aaggaaaaga gcagattatc tcctaaaatg actattacac tgacaactct ttatttacca |
| 87841 | gcaatactgt aaacaaaaag caagtagaat aatatcttta agatgccaaa aataaaataa |
| 87901 | atttcaatgt aaaattctat atctaactaa actatatttc aggtaaggga acctcaatta |
| 87961 | aaaataagag ttttattatc aaggactcca ctaaagatac ttccaaaata cgtacttcgg |
| 88021 | gagaaaaaaa taataatctc aaaataaaag gtcaaaatgt aagaagtagt ggtaagccaa |
| 88081 | acaaatggca atcataaggg tgaatctaaa caaactacct ttatgcaaaa aaatcataat |
| 88141 | attgtcaatt tatgagccta aaaagaaaac taaaatactg aacatcgata gcatatttaa |
| 88201 | gtcaggacaa caactatcta aatgaacatg tgccaagatc cttttatgtt gagaagaaag |
| 88261 | attaaaagaa tcatcttaat taaaatagca ttgtatacaa aattgagctt taaatacgac |
| 88321 | tccaaaataa ataccaatag ggatcttttt ttaaagttag taagccaatt ctaaaatctt |
| 88381 | aatagaagag caatggtgca ttaagagtca agataattct gaagatgaca aaaatgaaag |
| 88441 | gtatttacct tacctaatat aagtgcagag catacattta tattcattaa gaaagggtgt |
| 88501 | tcatggtgca gagatagaca aacaaaccag tggaacagaa tatacagccc aggacttaac |
| 88561 | atccccctgc aaatatgtgg aaacttatta gagagacaga attatagatc tgtgaagaaa |
| 88621 | gagtaagtta gtcaataaat gaaactggaa taaagtaggt ccttctttca cacatacaca |
| 88681 | aaaaaatcaa ttccatgtgg actaatggtt taaatgtaag agactaaact ttaaaatatt |
| 88741 | tccagtaaag ttatagcaca atataatgtt ataaaaggat tcttaagaca taaaacatat |
| 88801 | aaattatagg aaaagacaga ttgccaactt ctaaaactat tataaaataa aatgggaaaa |
| 88861 | taatccaaat acaagaagat atttgcaaca cataaaactc acaaaaatta ggtgccaaat |
| 88921 | tatataagga atgtctacta atcaacagaa aaaaaaagca caatagaaaa aaatgtgttc |
| 88981 | attctttgtt tataaaaaag atattttacc aagggaaaat aatagtggcc aatgaacaca |
| 89041 | ggaaaagatc cttagcctcc cttgtagaca aggaaatgta aattaaacct ccagtgaaat |
| 89101 | agtattttat atccaaggga ttggtaacaa atttaaaaat aaatatgaca atggcaaaag |
| 89161 | ttgatgagga tgtggagcag acagctacat actactctag aaatcaattt gatgttacac |
| 89221 | agcaaagttt aaaatgtgta tgcctatgac ccctggcatt gttattaaga aaaccttgca |
| 89281 | catgtgctct tataagcata tatttatagt accatggttt ataaaagaaa aaaaaaaaca |
| 89341 | gtaaaatccg tatatattcc aaagtctact tataggataa tgggttagtt atagatactc |
| 89401 | atagactgaa atactattca gcaatgaaaa tgagttaact atgtccagtc catgaacagg |
| 89461 | aataaatcta aaaaatataa tgttaatttt ttaaaaagtg agtcacaaga taacgatgac |
| 89521 | agtatggtat gtaaaataaa taaaactaaa ccatagattg cttaaggata aatatgcagg |
| 89581 | tagtaaaaac ataaggaaag tcacagagat agtaaaaaga aatatttaga actgtggcta |
| 89641 | actctgtgtg gaaaaggaag gttgtggtag gtggagcaca taaaaatatt tgaaagtatt |
| 89701 | ggtaatggtc tattttttaa cctgagtggt agcatatgtg tgtctattta ttaatcttct |
| 89761 | tcaaaagcta tgcatttcat attaactcac ttgtttaata taaaatattt tataatttct |
| 89821 | taagtaaaaa tgtcagctat gataaaattc attacatctc cccctccttc caaattatcc |
| 89881 | caacagttaa atgttgattc atttgtagag cattaaaaac acatttgatt gaataaaatt |
| 89941 | caattaacat atatgtagga gttcagtcag ggtggcggga aaagttgtaa gaaaaagtta |
| 90001 | tagggaaaga ctcaaacctt cttggaaggc caggaggttt tgcaaaagct tcgaaggaga |
| 90061 | aatggctgaa ggcagccaaa ttctgttatc cagaacctga aggcaaaggg tagataacaa |
| 90121 | gggaatgtaa aggaacttat ctagataagt tggtttactt atgtctctag aaaccaacct |
| 90181 | ttgatcattc ctgcacagga ctgctctcta ctcagggggt tggcaaagtt aattacccac |
| 90241 | aaattgtgtt tgctccaatc ctttgtcatt aaatctgtac taaataaatg caagtgtcac |
| 90301 | aggcttatgg gggctgcact ctgggttgca ctgcactttc attggcagtg ctaaggggtg |
| 90361 | cagtccatct agccatgctg tcaggcaaaa tacctgtgtc agagtacttt tgtcatccat |
| 90421 | cactcagcca gagtctgcgg gacagacttg gcacatatac ttaaaatacc tactaaaaac |
| 90481 | aatgtgttaa acctttttag gcccaaaaga taaaagatat aatatctttt taacatttta |
| 90541 | agttttgttc ttgagttagt tcatatatta agaagacgtg tctggggagg cagagcagga |
| 90601 | tggcaaaata gaaggctcca ccaatcatcc cccctgaaag gaccccaatt taacaactac |
| 90661 | ctacaccaaa aagcaccttc ataaggacca aaaatcagat gaccactcac agtatctggt |
| 90721 | tttaattcca cattgctgaa agaagcactg aagaggcagg agaaactgtc tttaattgcc |
| 90781 | agcgccaccc cttccccatc ctccagcaac aattccgtgt gctggggaga gggagagcat |
| 90841 | agcatttgtg aggaaatgaa ctcagtgctg cccttgttat agcagaaagt aaaaacaaac |
| 90901 | caaattcagc tgacacctgc ccacggaagg agcatttaaa ccagccctgg ccagagggga |
| 90961 | atcgccaatc ccagcagtca gaacttgaga tcccacaagc cttaccactg caggctaaac |
| 91021 | tgctctgagg ctgagaataa acttgaaagg caggtcactg cctttctagg tcactaggac |
| 91081 | tgcaactcct aggaaagtcc tagtcctgaa ctgggcccag agtcaatgga cttttggggc |
| 91141 | atgtgaccta ctgagatacc agccagggca gctaaggggg tgctggcatc acccctcctc |
| 91201 | tacccccatg gtgcatagct catggctcca aaagagacct cttccatcca catgaggaga |
| 91261 | ggaaaggaaa gagtgaaaag aactttgtct tgcattttgg atatcagctc aaccacagta |
| 91321 | ggctagggca ccagtcagag ttgtgaggcc tcccttccag gccccagttc ccagatgata |
| 91381 | tttctagaca caccctggga cagaagggaa cccatggcct taaacagaaa gacccagtca |
| 91441 | tatcaggatt catcacctgc taactgaaga gtccttggtc cctgaataac cagcagcata |
| 91501 | cccaggtact atgctgaggg ccttgggtga aactctgaga cttgctggcc tcaggtgaga |
| 91561 | ctcagcacat tcccagttgt ggtgactatg gggcgagaca aaaaagcaga gggaaagtaa |
| 91621 | aggggacttt gtcttgtacc ttaggtacca gcttggccac agtgtgggta gagtgccaag |
| 91681 | caggctcttg agttaccaat ttcaagattt ggctcttgga ccgcctttct gggccactca |
| 91741 | acttggaaaa gaagaagtca agtaactctt gtttgcagat gatatgatgt tatgcttgca |
| 91801 | aaagcaaaga ctccatcaaa aaactattca aattgataaa ttcagtaaag ttgcaggatt |
| 91861 | caaaatcaac atacaaaaat cagtagcatt tctatatgac agcagtgaac aatctgaaga |
| 91921 | agaaatcaaa agtgtaatcc catttataat atctacaaat aaaataaaat acctaagaag |
| 91981 | taacttaagc aaacaagtga aagatctcta caataaaaac tataaaacac tgataaaaaa |
| 92041 | attgaatagg aaacaaaaaa ttgaaagata ttacatattc attgattgga aaaaccaata |
| 92101 | ttgttaaaat gtccatacta cccaaagcaa tctacagatt caatgcagtc cctatcaaaa |
| 92161 | tacgaatggt gttcttcaca gatagaaaac acaatcctga agtttatatg gaaccacaaa |
| 92221 | agacccagaa cagccaaagc tatcctaagc aaaaagaaca aaattggagg aaatcacatt |
| 92281 | acctgacttc aaattatact acagaactat agtaaccaaa cagcatgata ctggcataaa |
| 92341 | aacagacaca tagatcaata gaacagaaga tagaacccag aaacaaatcc acacacctac |
| 92401 | agtgaactca ttttcaacaa aggtgccaag aacatacact ggggaaaaga cagtcccttc |
| 92461 | aataaatggt gtggaaaaac tggatatcca catgcagaag aataaaacta gatccctatc |
| 92521 | tctcaccata tacaaagatc caatcaagat gaattaaaga cataaatata aggcttcaaa |
| 92581 | ttatcaatct tctttaagaa atcactgggg aaattttcta ggacagtggt cagggcaaaa |
| 92641 | atttattgag taatacctcc caaacacaag caaccaaagt aaaaatggac aaatgggatc |
| 92701 | atatcaagct aaaaacttct gcacagcaaa gaaaacaatc aacaaagtga agagacaacc |
| 92761 | tacagaatga gagaaaatat ttgcaaacta ctcatctgac aaggaattaa taaccagaat |
| 92821 | atataaggag ctcaaacaac tctataggaa aaaaatctaa taatccgatt aaaaatgggc |
| 92881 | aaaagattta aatagacatc tctcagaaga agatatgcaa ataacaaaca ggcatatgaa |
| 92941 | aaagtgctca acatcactga tcatcagaga aatgcaaatc aaaactacaa tgagatatca |
| 93001 | tctcaccccg gttaaaagag cttatatcca aaaacaggca acaacaaatg ctggtgaaga |
| 93061 | tgtggagaaa agggaacact tgcacattcc tggaagtgta aattatcata ataactagga |
| 93121 | gaacagttct gtggttcctc aaaaaactaa aaatagagct accatatgat ctggcaatcc |
| 93181 | cactgctggg tatataaaca aaagaaagga aatcagtata tcgaagagat atctgcactg |
| 93241 | ccgtgtttgt tgcagcactg ttgacgatag ccaagatttg gaagcaacct aagcatccat |
| 93301 | caacagatga atggataaag aaaatgtggt acatttaaaa atgttaagtg aaataagcga |
| 93361 | ggcacagaaa gacaaacact gcatgttctc atttacaagt gggagatgaa caatgagaac |
| 93421 | acatggacac agggagggga ataatacaca ctggggcctg tcaggggagc tgggtgggtg |
| 93481 | gagggagagc attaggaaaa ctagctaatg catgctgggc ttaataccta ggtaatgggt |
| 93541 | ggataggtgc agcaaaccac cacggtacac gtttacctgt gtaacaaacc tgcatatcct |
| 93601 | gcacatgtac cctggaactt aaaattaaaa ttaatttaaa aaatcaaaac aattgaactc |
| 93661 | atcgagatag acagtagaag gatggttacc agaggctgga aaggatagca gaggaggtgg |
| 93721 | tgggtgggga tggttaacgg ctaaaataaa aagttagaaa gaattaataa gatctagtat |
| 93781 | ttggtagcac aacagggtga ctacagtaaa taataattta attgtacatt ttaaaataac |
| 93841 | taaaagagta taattgaatt gtttgtaaca caaaggataa atacttgagg gagtggatat |
| 93901 | tccatcttcc atgatgtgat tattatgcat tgcatgcctg tatcaaaaca tctcatgtgc |
| 93961 | cccataaata tacacagcta ttatgtaccc agaaaaatta aaaattaaaa aaagtcatgc |
| 94021 | atagctcata ttccaagagc ttttagaaat cattgttttg agaatgcttt tatataaatg |
| 94081 | aggatatatt gactaaaaat aacataactc cttataatgt actatatagg tatacattat |
| 94141 | aatatggcat atctccagct ctattttaat gtctctattt aatagattta ggtataattt |
| 94201 | tattgtattt tcagtaactc atttctaata acagaatttt ttaaatcagt aatgtcttat |
| 94261 | acattaagag gttaattatt tgatattcag tgttcaaaat ttgtttgctt taagactgat |
| 94321 | ttaaaacttt aaccaacatg tttgaaatca ttgagaccac agcccacgat cagaagacat |
| 94381 | actttagaaa atgattccta tttgtcctcc aagaaattta caaatatgtc cccatacaag |
| 94441 | agctccataa aggacttcat atccaaatat caagggcttt gaacaggata ctgccacatt |
| 94501 | acccgagccc taaagcattg tttacaagga attttgacac ccaagttaaa aaatgacaca |
| 94561 | cgagcaactt ttcccaagaa actattctaa aaaatgttaa attgtatttg gtaattcagt |
| 94621 | tgtagaaatc tgttttaatt tttcttaata gaaacataac aaagatcata aaaacactca |
| 94681 | ttttaattta caggtggcaa atattcattt tagtcaccaa ttattcaacc acaatgaact |
| 94741 | tctggaaaag caaagagtac taagttaaca aattgagtct attttcatca aattatctaa |
| 94801 | aatcagccaa aacttaaaga caatttcata ttcatgccca aaggccaaac acaagtttat |
| 94861 | aattcattgg gaataaaaga agtcctgttt tttaaaatcc ctcctgattt ttaagaaatc |
| 94921 | tgaaggaatt cagtttttat aaagccagct agtttcaaat agcattaatg ttgatgtggt |
| 94981 | aaattataga cgtcatttaa taaaatgtat atttagaaga caaaacagca ctatagacag |
| 95041 | aaagactttt taaaatgtgt gtcaaacaga agattagaaa aaagttttta gtaagaataa |
| 95101 | tcagaagtaa aaacagtgta actattttta actgaagtat tttaagtcct ttcttccaac |
| 95161 | aaaaattgag tgcctaatac agccatttcc agaacttttt cttttaatat gtagagagaa |
| 95221 | tatatgtatg aagccatata tatatattca tatatgtaaa accagataca aatattaatc |
| 95281 | ctcaagtgtg tatattacat tatagacatt tgataaaaaa ttataatgta gatattagaa |
| 95341 | acaacgcaaa tggctaatca taatatggta tggatgcact atattagtaa ttagatttag |
| 95401 | tctaattaca cattaaaacc ccattattat aggctcaatt ttatttttga agtgttttat |
| 95461 | taaagcagag gttttacctt tagtaggatg aagacataaa actttcataa gtgactaagt |
| 95521 | gtagagggat atttaacagg acccatgaca catccatata ctaatcacta atcgcacact |
| 95581 | attcatatct atttaatact gcagaacctc tggaatcttt ttgtttgttt gtctgtttgt |
| 95641 | ttagagacag ggtctcactc tgttgcctac ctaggctgca gtgcagcagc acgatcactg |
| 95701 | ctcactgcag ctttgacact gggggctcaa gctatcctcc cagctagttt ctttgttgct |
| 95761 | gagactacag ggactttcac cgtgcccggc taaatttttt atttttgttt tttgtagaga |
| 95821 | caagggtctt gctctcttgc ctaggctggt ctcaaactcc tgggctcaag tgatccttcc |
| 95881 | acttcagcct tccaaagtgt tgggggttac aggcatgagc taccacatct ggtcttgatc |
| 95941 | tttacttgtt aattatttgc tgcttaagag agaatcactt ttacttattc aagtgatttc |
| 96001 | aatgtatttc ttcagatatt ttatataacc attattaatg aggttttaat tattgcactt |
| 96061 | gttaataata tgcagaaaat aagagaagga attagagcta ttattcccat aaatggagtg |
| 96121 | aataagaaaa ataattttag aaaaacttga tcaattgtaa aatgaaattc acatccatgc |
| 96181 | tctctgaata tatgttaaaa taaaccaatt agtatttgtg gaggaacatg aatctgataa |
| 96241 | aaatttccat gccaaatgaa tatttctcat tatatcaaag tataaatctt cagtcttaga |
| 96301 | actgaaagac tttatttatt atctagctat gaatatatct tcattttgaa acatctacaa |
| 96361 | caaaaacaag aaagaagagt actcaatgtt atcagtttgg tagaactgaa ttaaggcaga |
| 96421 | ccttttgata tcaggttgac tctttcatat ttgttactgt attatccaca taaggtctta |
| 96481 | ctaggagaca aagttactct gccataatgc caacttgtaa atatttgaca ttgagtctca |
| 96541 | tagaaactct ctaaggacag tgaacaaata ccataagcag tactgcattt gagacaaaag |
| 96601 | ggaattaatt gagagtgaca cccatagata ccactggctg ctaagaagga tacgatcagt |
| 96661 | gccaggaaac agcactgcac cttttatcat ttctcccatg atgcacccta ctgaccacat |
| 96721 | gtcaactgaa aacaaaaatg aaatgaagat aaacaagaac acaggaatag aacaacaaat |
| 96781 | aaatgaaaac actgggctac tcatgccata gagcaatagt ctgacttttc actgactgga |
| 96841 | tgagaggcca gtggcatgat cattagcatt ttaaatggtg gaaaggggag aacataggct |
| 96901 | ctcaaggccc ttttctgtca tataacagaa ataacaacac tacccaatgc tacctaatgt |
| 96961 | agaataatat tactttgcaa ttataactca atgattaatt taggttttga tttaaaaggt |
| 97021 | ctaatttgca ttcatttact agaatcctac tgatttttat cataaattat tctagatagt |
| 97081 | gttaatatgc aaatcagttt cttttttact cttgtatacg tatttgaaaa aagaaaaaag |
| 97141 | aaaagctgag ctgccagcaa cctcaaaaga gtttctttta taaaacacat ttacccttat |
| 97201 | ttataaatat aaatacagtt ccagaagtga aaagccagtt caagaatgaa tgtttcagaa |
| 97261 | tatgttgtct tagtgtcatt gactctcatc aaaggagcat gtttctcagc aggaagcctt |
| 97321 | gtcttattct tataaacaca gttgccttat aatgataaga aaagctggct ttcaaaatgg |
| 97381 | gatcgctcaa tgtgaaataa acaacatgac taaagagctg atacttcttc tgaaactgtg |
| 97441 | aatcaaaaga aacctggaat tcaaggtgga attataatgg tctctctgtt attgccaagt |
| 97501 | atcatccaga acttaaaacc aggaagccag ggcagagcgc acagagggag gggcaaaatg |
| 97561 | gggaagggtg aagcaagaaa gtgaaatctc tctcttgagc tacacttggt tactcacttg |
| 97621 | ttgtacttaa aaatcagaaa gagagggcat tcttgtgggg ggaaaaataa acagaagaat |
| 97681 | tagggatgct gggggggagc aaatagggaa aggcattaag atagcagcac aatataattt |
| 97741 | tgctccactt acttttcaaa ttacatcttg taatttggca gtgaccttaa agagatcctc |
| 97801 | atgagattca ttccaatgtg aaaatctcac tcgggttaaa ctcaaagttt ccattgtttg |
| 97861 | atttaacacc taaacaaagt gatcactgca aagaaacggt tacggctttg aaggtgataa |
| 97921 | gcatacccaa agaagcaaat ttgttttgct tccagtttag gaaagcaact gacatgcaga |
| 97981 | aatatcaggg aaagatagag caaacagttg tgcatccctt tattattaac gtgtttgtaa |
| 98041 | tttcaagtaa tatctgtctc acaacaggat ggtattgaga aatctttgat taaagaaaaa |
| 98101 | aacggaaaac atatttattt ttaaattcca agtattccta tcatactgtc caaagagacc |
| 98161 | agtttgacta aattgctatt tatggattat ctatatttta tagatattgt ggaagagata |
| 98221 | tacaaaaaac attgagacca aaaatctcaa tatgcagtgt tttctcttgc ctgtctccat |
| 98281 | tcaaatgttt agaaaaaaaa tggtttctgc cattaacctg tctgggactt aggaaacatc |
| 98341 | tatttctatt tctcatttag tataaaattg ttttttacaa tatctctggc aatatattca |
| 98401 | tctttttctg ctagagaacc aattctatta ctttccaact atgtgacctt ggccaggtta |
| 98461 | cttctctcta aacctcaatt ttatccccta aaatattgat gataagctgt atttattatg |
| 98521 | tagagtttta taaaagactt gaaaaagtta tgtctgataa agatccttgc acaactgcct |
| 98581 | ggtacatagc aagtccccaa taaatattat ttacatatca aatcatcatg tattaaataa |
| 98641 | tatattaaaa ttatatttgg gaattaaata tctttggaaa aggtttattt attttaaatt |
| 98701 | tacataatta tattatatat ttacatctat aataaagaaa tctttccagt ggaggaatct |
| 98761 | caccactcac taggcaatct attttattgt tgaaaccact tagaaaggca tcctacatat |
| 98821 | tgactcacat tgactctatg gaacttctct tcactatgta aacaggaact actgtgtttc |
| 98881 | ctcatccaaa taagaacctt aaagtatgta actataacct gtaatcccta accattcctc |
| 98941 | tcctaaaact ggccaatttt atctttcagg ctaaaatccc ttgatttttt tttttttttt |
| 99001 | tttttttttt tttgctgttc ttcacatgcc tttaaacaac agcaacaaca ataacaacag |
| 99061 | atggcctaaa ttaaattctg gtctctatgt tgctttagca ccagttggtt ctatacagtt |
| 99121 | gttaattttt ttttttcttt gagccagtgt ctagctctgt cactagactg gattgcagtg |
| 99181 | gcacgatctt ggctcactgc aacctctgcc ccctgggttc aagtgattct cttgcatcag |
| 99241 | cctccctagt agctgagatt acaggcacac gccaccacgc ccagctaatt tttgtatttt |
| 99301 | tagtagaggt ggggtttcac catgttggcc aggatggtct tgatctcctg accttgtgat |
| 99361 | ctgcccacct cggcctccca aagtgctggg attacaggca tgagacacca tgcctggccc |
| 99421 | agttgttgat ttaataactg gtattaataa ctggtatatc tcacagttct ctttttggtg |
| 99481 | tgggggatct gggagatact ccaatgtaaa agaagaatat ttatttaaaa tatatatctt |
| 99541 | catgggcaga actcaaaata gatttaatag atcacaaatc ttttgcataa aaataatgca |
| 99601 | gagatgtagg ctctagatct gaaattctga tgtacatatt gaatttcatt ttctttattt |
| 99661 | taatatatct ttatgggtga taatatcctg gaaatctttc taattcccat tttaacttta |
| 99721 | ctcaccattc actattccta gagaatattt atatctatgc taccaaaatg ctaagccttg |
| 99781 | tgcctagaaa tgataagctc tgttatcttg aattacaaaa attgaatata tgcacataaa |
| 99841 | tttatataga tttttatgta ttcaagacag gattatggct caagataaac tcattgtacc |
| 99901 | aaacttcatg aatttgccat ctccactaac agaaaatgta taataaaaga atttgatacc |
| 99961 | aactcttatt gaacaggaag gtagtagtag taagccttcc ctctactaat atctctatat |
| 100021 | taggatacat aaacatacta atacaatatg tatctgaata tattagtgat atatatatat |
| 100081 | atatattgct aatgaagtta agaatagatt attttatgaa attgtcaggt attacaatga |
| 100141 | atagggactg tgcatagacc ttagaagtaa ttagcaaatt caatcactgt tggaaactgc |
| 100201 | attttatgct ttctgaaaca atctgataca gcatctgggt ctctgtaaga gttctgtaca |
| 100261 | ctaatgctgc caaggtagaa ttctgttggc ttttaaaatt ctgattcatc acattctttc |
| 100321 | atcatcattc tatcattccc ataggcagaa ggcagattca cttgcaactg taacaatcag |
| 100381 | atgctgagtc actttcctta gaagaaatgt tgagtaaatt ttggtgttac caaaatatga |
| 100441 | ttaattttca gagtgagaac ctctgaaaat ctcatgtcac attatttgtc agaaattagc |
| 100501 | ttataataaa ttatctttct tctacttcta accaagagat agaaattata caatgtaagc |
| 100561 | cctggaagtt aaatctaaat aaataaagaa ctatattgct acttccacac tccatctaat |
| 100621 | agcttaaatt atgatccaca attaaataat tttgagacta tgtgagcttc tatcctggaa |
| 100681 | aatgtttcct ctctggggga ttacgtaact cttttatcac aatttctgag acttctcctt |
| 100741 | aacacaccta agtatatttt catttagtgt agaatttaga caaattttct ctcaatgaat |
| 100801 | gactcaacac acaatagcaa gaagatttaa tatagttttc taaaatcaga acatttcttt |
| 100861 | gaaaattatt tgttataatt tttataaaaa ttataagtta taaagttagc agttataaaa |
| 100921 | ttagaacatt tcttttaaaa ttagttaact aagtcataaa tttagttgtt taacaaaagg |
| 100981 | accttgtact aaacttcagt tcaaagacag aattctaaaa gaccactgat atacataaat |
| 101041 | aaactaagaa tgtacatatt ttgcacatgt gaatctaagt aagaaaaata agcaacaatt |
| 101101 | cgcagaactt aatgctaaat attgtaatgc caggaaatca gaaaaagact ttctccttga |
| 101161 | atctacctat taaacttgct tttatttctt tacaacctca gaatctccag ctcccattct |
| 101221 | ggtgaaccct gcctcacatt ttcataaaat aataacaaaa gagaagagtt agcaggcaca |
| 101281 | ctagtggtaa aaatgtattc ttttaaacaa attcaaataa taattttatt tttaaatatt |
| 101341 | tatttataat tacatgcaat ttgcttatag atttgaaaga atgcctacta aattgtttca |
| 101401 | tcctaacttt atttaaaacc ataaatttta tattgtttga aataactctg tattgtctta |
| 101461 | attctaagaa aaagagttag actatatagt aagttgttag ttgcaaaagt ttattaggaa |
| 101521 | ttaagcacta ttatttcaaa gaggtaagaa gaattttaaa atatctctgt gctagattga |
| 101581 | tataacaatc tgtaaccaca aatttttgtt cattttactt tgatccttcc taatgttcat |
| 101641 | attcaaactt taaaaagata cagcattcag ttaatttgag ggtgtttttc ttatttcttc |
| 101701 | aaatatactt ttggaaaacc atttacctat tccttaataa cttggctcct tggcaaggtt |
| 101761 | atatgtatat ctctttcatt ctaactgcaa tttcctgttc ttcagaggga aaggcaagag |
| 101821 | gagctatttt agcagtccag ctgttagctt tggatataat atacttgagg tccagaattt |
| 101881 | tcaaatgcat gcagtatgcc tgtctttaac tttatatcca gagaatattg gccaagaact |
| 101941 | tagataaagg gacatcacag ggtttgctca aagaaggtgc tatcatttga agtagaatgg |
| 102001 | aaaaaataag gttgaagaat gtatctaaaa gaagcaggtt tgaggaaagg actcatgaaa |
| 102061 | attatactgg gactatagga gtctttgata atcaacaaga tggcatgtcc tggttggggt |
| 102121 | ttggggtggt ggaggcagtg aatgactgga tagttcttag aaaaaacttc taaggaagca |
| 102181 | ttgtaaagct gctttcctgt aaattttatt gaaaggaaca aaatctgagt gaagctaaag |
| 102241 | tcactgattt cttttatctg ttctttggac tacataaaaa aaaataatgg acttggccta |
| 102301 | tttactactc tctaacttga aagtcagcat gcgtttaaga atgtttaaag aatgtttagt |
| 102361 | aacatattta aatatgaatg tgcattataa tgttcaaata aactacatta tgtaaaaata |
| 102421 | tatgctatga aatgttttaa tcataaaact atagtgaaaa ttaagtgtat gatagtgtat |
| 102481 | gcataattaa gaaacacttt acacaagaaa aataagtatt aagaagtgat cctctggata |
| 102541 | ttaaagtaat tagcaaatta ataagtggaa aagcaaatat gtattgttta ttttgttcaa |
| 102601 | ttttattttg ccaacattct aattcagtaa ccaagacaat tccctgtgat atcatctaga |
| 102661 | tcatatcaac caccaagaaa cccccacgat gtttctcaaa atgcaaagat agtttgaatg |
| 102721 | tgggaaagta gttaaacttc agtcatcaat gacaatttca taagattctc tgatttgtca |
| 102781 | agacatttta ctatttttgc ttagggttca agtttaaaag ttctgtgaag agagaagaaa |
| 102841 | aatgttgatt attatgatag tattatcaca tcatttttca gcctcaaagt aatttaatat |
| 102901 | aaattactct gaataattct aggtgaaaga catgagaacc tctgattgtt ttttgaaatt |
| 102961 | tctgcagagt cctggaagta gtcaaggtat caattagcat atacttagct ttgatatttc |
| 103021 | ttccaataaa tgttagacta atttcaatat gcttaaaatt gtgtaattct tctgccgata |
| 103081 | aataacttga atcagccctc agccttgtgt ctgatagtca tagtttgaga ggttatctga |
| 103141 | ctgctcagtt gttggtagga agtcctgtgc ataaaatgat aataatttca tttaaagatt |
| 103201 | aataaaacaa ttctactcaa aattaacaaa gcacattgtt taactgagat ttctataggt |
| 103261 | ttatttttta aatgtaacta taactttaaa cattaagtta taattttaat tataggtaaa |
| 103321 | aggggctaag aaatccaaat ggaaacattt tcaaaaagga aacatttttt ctaacctatt |
| 103381 | tcattggaaa atatctactc attttttggt aaaatttaac ttaagtaaaa ggctttccat |
| 103441 | tgtaggtgtc tttatgcaat tttttttaac ttttaggtcc acatgcaggg tcattatcac |
| 103501 | atatggcact aggctatcaa agactgctgt actatgtaat catcacttga gataaagaaa |
| 103561 | tatatatatt tatataataa atgcaaacct ttattataat ctacattatc tgtattagag |
| 103621 | attatttaaa tgtctaggtt catacatatc aaggatataa atgttatgta ttaattttca |
| 103681 | cacaatttaa tttcagctcc aaaaattttt ctaaaacaaa ctaggattaa aaaaggtggg |
| 103741 | aggggcacct ccaagagaaa aatttttaaa agaaaataaa attatcaata tggtgctttg |
| 103801 | tttttttgtt tgtttttctg aagggttaga ttgagtttaa aactttttgc tttttttctc |
| 103861 | ttttattggc ttttaaattt aatattgtca aaatagaaga aaagcaatcc gcataaatgc |
| 103921 | ctgaagagct aaataagatc tcagagaaca acctcgctac atgaaatcaa gtgacatgta |
| 103981 | acaaaatgtc acatggaaat gagaaatcct gtggcttttt gctttccact gacaagtgca |
| 104041 | tgagagagat gtggctgtgc aacttcactc tgagtattga ttgagaggtt ccttttttgt |
| 104101 | ctaaaatgtg ttatatacca aaggcaaaag cataaagaaa ataattccta cttttgaata |
| 104161 | aagtcagcac atttaaatat gaatgtgcac tataatattg ttcaaataaa ctatattagg |
| 104221 | tatataatta atccccaaac tatagataag taaagtgaaa taaattttcc taagtagcca |
| 104281 | tatgaaatct aatggtgaaa atgatacttc acactataga taggaaagga acatggagca |
| 104341 | aacagagatt caagacctta agattggtta taaataaatg gttgccatgt ggttttctta |
| 104401 | tcttctagac cagcactgtc caatagaaaa atgacagtca caaatgccaa tcatatacgt |
| 104461 | agttttaagt ttaatgtcca cattcaaaag aataaaaaaa gatatatata tttaacccaa |
| 104521 | tatattcata atgctatcac cttaacacaa gctatataag attattagta atatatttta |
| 104581 | catgctttta aaaatatctt caaaatctgg tatgcatttt acacataaaa cacatttcaa |
| 104641 | tttagactat aatttcaagt gctgaataac cacatgtggc tagtggctac ctactagaca |
| 104701 | gtgcagttct agaatatgtc atcactctta cattactagt ggattttaga gactatctca |
| 104761 | ttaatatcca gcagaatgaa tacataccta cctaaataca agcttattat ggtacccact |
| 104821 | tcttgttgag tcaggttttt ttgtttttgt tttttactat tggacttttt aaacacctca |
| 104881 | ataaaaatca taacctttct tacataatag aaggattcct taataaaact ttcttatcaa |
| 104941 | tcaaatagaa tggttaatag agttatgggt cacatggtta ttgccaattt ccaaaacatc |
| 105001 | catatgtaat aaatatacta aattcaatcc tgtattataa ttgttaaagt tgtcaatggt |
| 105061 | gattgtactt agatttgcac tagtatttgt agtaaggtat ttttgtattt ttaaaagtat |
| 105121 | attatactat tatatgtaaa ctttattgtc tatatattac aaacacaaat ctgtaccttt |
| 105181 | actagagagt gaatttttgg ccatttaaga aatgtatcag ttcatttgat aattccaaat |
| 105241 | catagatagc tctacaagaa atttctgatg gactctattg caggactcag tagtagaact |
| 105301 | taaatccaaa ttgctttatg aaattataaa tttagttggg aaaacaagca tgtatgtcta |
| 105361 | tgtcacatct ccaagtcagg tgagagaagt ctacattaaa ctcacctgac cactagcaac |
| 105421 | ttgaacaata attacaattt ttaaatgcag aagttcatgt tttgaggaag atatgctata |
| 105481 | ttgacctgtt aaagacttga tttagtgtcg agtaatttaa atcaacaaag ccatagacag |
| 105541 | aagcttagaa tgtcagcaag gagagcaatt attatgccta aatattgtag aagcacaaaa |
| 105601 | tagttttaga aaggagatgg aaaatgactt gcaaatagtg acttattttg ctagttataa |
| 105661 | aggtaaatat attttaatca tttgggatct ggtagccttt gttattaaag aaaaatagca |
| 105721 | tttctatttt tagttgcagg caagaaagta attttagaag aaaaaagccc attgaaattt |
| 105781 | aattaaaaga aatcattgtg gtaggcagtc ttcagtactg gtcagattat agaaattatc |
| 105841 | acttttttat tacaataaca ccacttaccc aactttttgc ttctatcttt actctcctgg |
| 105901 | taaaatcatt atgtgtttaa aagggagaga gaacaaaaca ggtaaagctg tagcaaaagg |
| 105961 | tacaaggata cagtcccttc ctggaaagag gattttgtgg cgaaccattt ctcccataat |
| 106021 | gcatcccaca gaccatatat ccactagaac aggagagaga gggtagtgaa gaaaagggag |
| 106081 | gaaagtaaag ttaactaaga taattgactt ctgaaggagg aggaaaaaga acagtttgat |
| 106141 | taaaagtaca attttcacct cccaccaaaa gttggccaaa atgtgaattc aaaccagcgc |
| 106201 | tatgctatta cataaacagt gtttagtatg agaactcaaa ttaatgttta aaaacccatt |
| 106261 | actttgtggg tgccttacat tgcctctcag cagtgccaca aagtggtgac attggtgact |
| 106321 | gagaaaagca atagctaaac caaaagtatc tccatggcag gtgacagcta aaagccacca |
| 106381 | aagggttttc tgtgtcattt tccggatgca tagaaaacaa tattaacttg tagagttggc |
| 106441 | tattttcatt ttctattcta ttcagttaca gagaataggg caacaagaga ccaccttttt |
| 106501 | tttcaggggg ccagggggcc ggggcaaaac aagataaatt caaccaaatc caataaacca |
| 106561 | ggaaatgact ggaaccatgg tttagaggca aaagaattgg aattcctggt atttgggact |
| 106621 | tagcttttcc ttcagaagtt tattcttgta aataaacatc agttttagtt tccatttgaa |
| 106681 | atatttcaag aaaaaagaca tatgatgaat tagcacatgt ataaaggagt catctggaag |
| 106741 | gaaagtgaaa aaagtattaa aggtgttaaa aattgttatg tacatatatt tatttccaaa |
| 106801 | ttgtcaaact tgttaggaaa acatgctttg ccttctctta ccaatcttta cccctttcca |
| 106861 | acttgctttt cattatggac aaagggaact tttgggcaga atcatttggc cgaattgttc |
| 106921 | catggggaag gaaggcgggg ctaggtgcag gggacagcag ggcatgcctg atgtgggagc |
| 106981 | aagcaggttt ttctgcatga taaccttcct cccattggtg ggagggcagc accagggtgc |
| 107041 | taggcaggcc tcctccatgg acttagagaa gtcaaaaacc taaggcagca acttgcaatt |
| 107101 | ccttctagtg agggaaggga aagggatttt aataccagct attggtagtg ccaagatctg |
| 107161 | aaagcccaaa taggcaaatt cagaccgccc caccccccag tctcctgtac attcttgatt |
| 107221 | cttgactttg tttctccttt gactttttaa aaataactcg tgaagaaagt ataaaaatgt |
| 107281 | cttcttttct gaataaatta gatgtggtat tgcctagcaa ggagggctag attcatctct |
| 107341 | tctgagtcaa aaggtaccag aactccgata ttgcccacca gggtgaacct gacctttgta |
| 107401 | agaggtgact ttttaacttc ctctgtataa atgcatacat acattatctg tcttctaatc |
| 107461 | taagagtata caaagaagtt tctgtaaact tcagtggcca gggaattttc acaaaagtac |
| 107521 | cctgtagcac ctgtacataa aaatacactg attattttta taaaacttat taatacagac |
| 107581 | tgctaaaaac taaatagaat agaatataaa aataaatatt aaagatacac tgaatacata |
| 107641 | tacaatccta atttaaaact attttaattc ctaagtatat tgaatatgtt ttcaatatgg |
| 107701 | tgcattactt gtaagatata gtctcatcaa aataatacat tgcaatgctt tctaacatat |
| 107761 | ttaataaatc cttagtttta atcaactaca ctcaattgtg atacattttt aagattcctt |
| 107821 | ccatagcctt cattttaaag tgttcatgaa cattaagttt tctttatgaa agcagtagtg |
| 107881 | tcaaatttga cattttcctt ttttacgatt tttggaatat ataaggattt attaggcaat |
| 107941 | tgctactcta aatgtttctt acagagcagc ttaaattatt acagaaattc ttattcagtt |
| 108001 | taaataattt ggattatttc aaagttgtct atgaagattt tcactctaag gatttgtgtc |
| 108061 | acttttttgc ttgtcttttt ctagaactac agccatcagt ctgagataca acataaaagc |
| 108121 | cacatggcta actgtggtag attcctttct gctttcacac tcatgctcag ctgagctgca |
| 108181 | acccaaatag caatttttct caatatctag gcacgtgtgc taatcagcat ccgcttgact |
| 108241 | gtttaatcag actccattta caattctttt tcactattaa catttagtgc tcttctagca |
| 108301 | aaactgactt ctcaataaca tccttcacat ttttaatgag tgtttcttac cagtagtatt |
| 108361 | aaaaaaaacc ctgaatatgc tatctggcag ccctacttta taaaagcatg tacataaaag |
| 108421 | agaacattcc ccttggctat ctacaacgtg cacccgtttc attcatggtt ttgatgctgc |
| 108481 | tctcccgaag catccctacc gttctccttg tagcccatcc ccaggatgac ctcaggggct |
| 108541 | ctgtaataac gtgtcaccac atatggagtc atcatgaagc ttgtgcctgc tgtcctggcc |
| 108601 | agtccaaagt ccaggatttt caatgtgcaa tcagacttga ctacaatgtt acttggtttt |
| 108661 | aaatcctaac agtaaggata agggaaaaaa ttaaaagcca gtatcaagtg aaatgttatt |
| 108721 | tccattgact caatcaacat agccattttg cagtagcact gatgaggtcc cccttgccta |
| 108781 | cagagctcta aataaggcaa atgattactg ctacaaataa gttaactatt agtttttgga |
| 108841 | aagtgtttta atcccagaaa agaattgtat tatcattttc acaatttaca ctaatcttgg |
| 108901 | gaagaggttt tagaaaaaaa aatatttatt gtcttctgtt aggtttctcc ttaggacaac |
| 108961 | ataaatcttt caacatatat atcatttcta ggcctgacag aagaacagat cagggaagct |
| 109021 | gtctttatgc tgccagaaac atctatggaa atgaattttc ttatttcagt tgcacatatg |
| 109081 | acttctgcca atcaaggagc tgaaagcatg aggcttctaa gaagctgcat ctcaatgagc |
| 109141 | caaaacataa cattgaacaa actcaactac aatataacta gaatttcctt ctacaattag |
| 109201 | tgaactaggt gctaattcta gtatcaaaga aatgaggaag tatattatat ttgaccaatg |
| 109261 | ccccccgtat aaagaaaata atctacagtt actcttcctc ttaaagcatt tgaattgtaa |
| 109321 | tcctagagaa ggtgttctta ccctgtgaat aattccagca gaatggaggt gcttaatgcc |
| 109381 | acacaacatt tggtacagca ggtaagacat tcgctcatgg tctaattcca tctgaatcac |
| 109441 | ttgacataag ttggcatcca tcagttccat tactaagtaa ctagaagggt gaatccacag |
| 109501 | tgttagttcc agatcacaag atctatgaag tcctttgatt tagtcagaat gctctcctaa |
| 109561 | ctctgtaagt cttctgaact tcttagctct gcctttgact taatcaagac tgagcatttc |
| 109621 | caggtattgc atgtgtgtta gctatacgaa ccctaccaaa aggttgctag tagaaaaagc |
| 109681 | atttattaat gtctggttca tgtccacaga tttgttacct taattttgct ttcactttcc |
| 109741 | cctgtattcc ctcaactctt ttgtgaatat tataatttca aataagaaaa aaattaagga |
| 109801 | cagcttcttc cattaaattc aatgtatctg tattaaagac aaggtgctag ggaatcactt |
| 109861 | cctctcctcc atctgttgtc actactgggt agactttgca caaaatcaat gacactataa |
| 109921 | agatagaaag aataaagata attttcacgt cccttttatt actatttctt tttgtttgtt |
| 109981 | tgttttgaga cggagtctcg ctccgtcacc caggctggag tgcagtggta cgatctcagc |
| 110041 | tcactgcaac ctccgtctcc cgggttcaag caattctcct gcctcagcct cctgaatagc |
| 110101 | tgggactaca ggtgcatgcc accacacctg gataattttt gtatttttag tagaggtggg |
| 110161 | gtttcaccat gttggccagc atggtcttga tctcctgact ttgtgatcca cctgcctcgg |
| 110221 | cctcccaaag tgctggaatt acaggtgtga gccaccatgc ctggccttat tactatttct |
| 110281 | tcaggtaaat taactcattg tagaaagttt gtggaatcta ttaataaata taaataaata |
| 110341 | agccttccct gagtgaccaa gagcatggaa caacaaagta tataaagcca caaggccaaa |
| 110401 | aggaggcaga atgtctttcc aggaagtaac cccacaaaca gaatcttctt tacatatcac |
| 110461 | tggacccctt tcatttgacc ccacctctta cctctctttg agcagtataa gggatttcaa |
| 110521 | ctctgtgtgt gtgtgtgtgt gtgtgtgtgt gtgtgtgtgt gtgtggtgtg tgattttccc |
| 110581 | ttgggctatc tgagtgctgt gctctccctt ccttcatgag ttttgttact tacacatctt |
| 110641 | ggaactcctc cagcgttttc tggggtgtga agacatttaa taaactaata atctgaaaga |
| 110701 | gagYggaagg gacagaggaa acgagagaaa gaaaaaagag agagaatgta ttatttttaa |
| 110761 | attgtatttc aactcaaagt cactcactag tgctatggca gaaatggaga agttggaaaa |
| 110821 | atgtcttgag ctgtctcaga ggacacagaa aagtatgctc agaaagaaca gatgatctgt |
| 110881 | cttcaggtta ataatgcagc taaaaagctc aattcttggg cagaagcaag agtcagagag |
| 110941 | atgcataatt |

Following is a MAPK10 genomic nucleotide sequence that includes the MAPK10 SNP “MAPK10-AA” (SEQ ID NO: 4940).

AGCAAGGCAGATGATGGAACAACAAAGAGAATCACCACTGTAAAAGCC
CCCCAGTCCTTTGCCCACTAGGTCTGCTTTCCTATAATATGTCTAGGAG
GTGATGATATTATCAAATTCACAGAATATAATATGTTCTGCATTCACTT
GTCTGTCTTAACACACACACACACACACACACACACACACACACACAC
ACACAC[A/C]CCAAATATTTCTTAAATAGTCACCTGTTATTTCCCAA
GAACATCCCAACTTTTCCGGTAGGCAAGTGTCAAGTCACCTGGACAAT
ATTCCTGAAAAAAAGTGAAGGTTCTACCTGGGGTTGGGGCTCATTTTGG
AACATGAAGTTGGCACTCTGAGGAGCCTAACCTTCTGTTAGGCTTCCTT
TGGGGCAGTTGGTTTGC

Following is a MAPK10 genomic nucleotide sequence that includes the MAPK10 SNP “MAPK10-AB” (SEQ ID NO: 4941).

AACATAAAACAATTCCATTTAGAATAGCATCAAGAAATACATATTTAGG
AATAAAGTTAGCAAAAGAAGTACAAGTTTTGTACTCTGAAAACTACGA
AACATTGTTGAAAGAAATTAAAGAGGATCTAATAACTGGAAAAACATT
TCATTGCTCATGGATTAGAATATTTAATATTGTCAAGAAATGACAGATA
CTCCCC[A/G]AATTGATGTACAGATTAAGCCAATACTTATCAAAAT
CATAGCTGTCTTTTTTGCAGAAATTGACAAGCCCCATCTTAAAATTTG
TGTGGAAATGCAAAGGATACAGAATATCCAAAATAACCTTGAAAAAC
AACACAGCTGGGAAATTTACATGTTCTGATTTCAAAACTTACTACAAA
GCTACAATATTCAAGACAGAG

Following is a MAPK10 genomic nucleotide sequence that includes the MAPK10 SNP “MAPK10-AC” (SEQ ID NO: 4942).

TTTGATGAGGCACATTTACCCTTTAGCCCATGTTAACATTTTCTTCAGGA
TTCATTACTATTAAAATTATTTATGAAAAAGTTTTTGTACTGGATCATTA
CCATCAGAATAATCAGAATGAATGCCACACTGAATATCAAAAGAAATA
AAACTAAAATCATTATAAGGACACAACCATGTGATATTTGTCCATCTGC
TCTTTAAGCAATGTTATGTTATTTCTTGCAACCCCTACACAAAGGCCAA
GAAATTACACAAGTACTAGTTTATTGGTTATTCACGGAGAGTGAGTACC
TGAAGGAGAAGGCTGTCCTTTTACTACACCATTTTTAGTCTTTTCTTCTG
AATTCATTACTTCCTTGTAGATAAGTTCTGTAAGAAACAGCTGTGTTATT
ATAGAAAACAAATTTATCCTTCATCCACAGGGAAATTCATTACTTAATG
CCAAATAATTACGTTTTGATGGGGTTGAGTAAATTAAAAATCA[A/G]T
TGAGATTCCTTTACTGCTCTATTTCTATTTCTATATTGCCTTGTAATTAG
CAGAGAGCCTGGCACAAATAGGTATCTAGTAAAGCAGTTGAAGAAATCA
ATGCATGAAAATTTATTTCTACGTACATGAAACATACTATTTATTTCCAT
ATACATAGTATTTATTTCTACATACATTTCTACATAAATAACATTTATTT
CCTAATGCATGAAATATTTCTACATAAATGAAACAAGTAATACATGTT
TTACTTGTTTTCTGAATTCTTTGGGGGAAGTACTAATACATTCTAGCT
CAATCTAGCAACAATTTTTATGGCTTTGAACATCCATTT

Following is a MAPK10 genomic nucleotide sequence that includes the MAPK10 SNP “MAPK10-AD” (SEQ ID NO: 4943).

TTTGATGAGGCACATTTACCCTTTAGCCCATGTTAACATTTTCTTCAGGA
TTCATTACTATTAAAATTATTTATGAAAAAGTTTTTGTACTGGATCATTA
CCATCAGAATAATCAGAATGAATGCCACACTGAATATCAAAAGAAATA
AAACTAAAATCATTATAAGGACACAACCATGTGATATTTGTCCATCTGC
TCTTTAAGCAATGTTATGTTATTTCTTGCAACCCCTACACAAAGGCCAA
GAAATTACACAAGTACTAGTTTATTGGTTATTCACGGAGAGTGAGTACC
TGAAGGAGAAGGCTGTCCTTTTACTACACCATTTTTAGTCTTTTCTTCTG
AATTCATTACTTCCTTGTAGATAAGTTCTGTAAGAAACAGCTGTGTTATT
ATAGAAAACAAATTTATCCTTCATCCACAGGGAAATTCATTACTTAATG
CCAAATAATTACGTTTTGATGGGGTTGAGTAAATTAAAAATCAATTGAGA
TTCCTTTACTGCTCTATTTCTATTTCTATATTGCCTTGTAATTA[G/A]C
AGAGAGCCTGGCACAAATAGGTATCTAGTAAAGCAGTTGAAGAAATC
AATGCATGAAAATTTATTTCTACGTACATGAAACATACTATTTATTTCCA
TATACATAGTATTTATTTCTACATACATTTCTACATAAATAACATTTATT
TCTACATGCATGAAATATTTCTACATAAATGAAACAAGTAATACATGTT
TTACTTGTTTTCTGAATTCTTTGGGGGAAGTACTAATACATTCTAGCTCA
ATCTAGCAACAATTTTTATGGCTTTGAACATCCATTT

Following is a MAPK10 genomic nucleotide sequence that includes the MAPK10 SNP “MAPK10-AE” (SEQ ID NO: 4944).

GGTATCGACAAACACTGACAATCAGTACCATTTTAAAAGGTATCAATGG
TAAATGCCTTTCATTCTTGAGCAAA[T/C]GTGACATTGCTTTCGGCAT
GAAAAGACAGATGAAGTTTTAGGAGCTTTAAAAATGGTCTAAGAGAATA
GCAGTCAGCAAATCATTTTTTAGTAAATTGTTTTACAAGGAAAAGCCTT
GTTGATACTTTCTGAGTTCAATAAAATAAAAAGTATACTTTTTTAAGTAG
TAGACTTACCTTTCCATTCTTCAATTGTGTGTTCTCTTTCATCCAACTGC
TTGTCATATATCTGAGGTGGAGGCTGCCGAATAAAAACAAAAAATAAT
CTTTGTGTGATAATGTAAACATAACTAAACTCTTACAATGCACGAGAAT
ACCTTGCCCTCATTTATTTCGGTGATTTCAACCGTATATAAGTTATGAG
GAAATTACAGTAGTTCAGAAGTTAAAATTTTCCTGTAAACTTATGGAGG
ATTCATTCATATTGCAATATTTGCTGCTGACCCAATATTGCTGCAATAT
TTGCAGCAGAACGCAAATCCCGAGAGCTCACTTTCC

Following is a MAPK10 genomic nucleotide sequence that includes the MAPK10 SNP “MAPK10-AF” (SEQ ID NO: 4945).

TGAGGGCAAGGTATTCTCGTGCATTGTAAGAGTTTAGTTATGTTTACA
TTATCACACAAAGATTATTTTTTGTTTTTATT[C/T]GGCAGCCTCC
ACCTCAGATATATGACAAGCAGTTGGATGAAAGAGAACACACAATTG
AAGAATGGAAAGGTAAGTCTACTACTTAAAAAAGTATACTTTTTATT
TTATTGAACTCAGAAAGTATCAACAAGGCTTTTCCTTGT

Following is a MAPK10 genomic nucleotide sequence that includes the MAPK10 SNP “MAPK10-AG” (SEQ ID NO: 4946).

GGTATCGACAAACACTGACAATCAGTACCATTTTAAAAGGTATCAATG
GTAAATGCCTTTCATTCTTGAGCAAATGTGACATTGCTTTCGGCATGAA
AAGACAGATGAAGTTTTAGGAGCTTTAAAAATGGTCTAAGAGAATAGC
AGTCAGCAAATCATTTTTTAGTAAATTGTTTTACAAGGAAAAGCCTTGTT
GATACTTTCTGAGTTCAATAAAATAAAAAGTATACTTTTTTAAGTAGTAG
ACTTACCTTTCCATTCTTCAATTGTGTGTTCTCTTTCATCCAACTGCTTG
TCATATATCTGAGGTGGAGGCTGCC[G/A]AATAAAAACAAAAAATA
ATCTTTGTGTGATAATGTAAACATAACTAAACTCTTACAATGCACGAGA
ATACCTTGCCCTCATTTATTTCGGTGATTTCAACCGTATATAAGTTATGA
GGAAATTACAGTAGTTCAGAAGTTAAAATTTTCCTGTAAACTTATGGAG
GATTCATTCATATTGCAATATTTGCTGCTGACCCAATATTGCTGCAATA
TTTGCAGCAGAACGCAAATCCCGAGAGCTCACTTTCC

Following is a MAPK10 genomic nucleotide sequence that includes the MAPK10 SNP “MAPK10-AH” (SEQ ID NO: 4947).

GGTATCGACAAACACTGACAATCAGTACCATTTTAAAAGGTATCAATG
GTAAATGCCTTTCATTCTTGAGCAAATGTGACATTGCTTTCGGCATGAA
AAGACAGATGAAGTTTTAGGAGCTTTAAAAATGGTCTAAGAGAATAGCA
GTCAGCAAATCATTTTTTAGTAAATTGTTTTACAAGGAAAAGCCTTGTTG
ATACTTTCTGAGTTCAATAAAATAAAAAGTATACTTTTTTAAGTAGTAGA
CTTACCTTTCCATTCTTCAATTGTGTGTTCTCTTTCATCCAACTGCTTGT
CATATATCTGAGGTGGAGGCTGCCGAATAAAAACAAAAAATAATCTTT
GTGTGATAATGTAAACATAACTAAACTCTTACAA[T/C]GCACGAGAA
TACCTTGCCCTCATTTATTTCGGTGATTTCAACCGTATATAAGTTATGAG
GAAATTACAGTAGTTCAGAAGTTAAAATTTTCCTGTAAACTTATGGAGG
ATTCATTCATATTGCAATATTTGCTGCTGACCCAATATTGCTGCAATAT
TTGCAGCAGAACGCAAATCCCGAGAGCTCACTTTCC

Following is a MAPK10 genomic nucleotide sequence that includes the MAPK10 SNP “MAPK10-AI” (SEQ ID NO: 4948).

GGTATCGACAAACACTGACAATCAGTACCATTTTAAAAGGTATCAAT
GGTAAATGCCTTTCATTCTTGAGCAAATGTGACATTGCTTTCGGCAT
GAAAAGACAGATGAAGTTTTAGGAGCTTTAAAAATGGTCTAAGAGAA
TAGCAGTCAGCAAATCATTTTTTAGTAAATTGTTTTACAAGGAAAAGC
CTTGTTGATACTTTCTGAGTTCAATAAAATAAAAAGTATACTTTTTTAA
GTAGTAGACTTACCTTTCCATTCTTCAATTGTGTGTTCTCTTTCATCC
AACTGCTTGTCATATATCTGAGGTGGAGGCTGCCGAATAAAAACAA
AAAATAATCTTTGTGTGATAATGTAAACATAACTAAACTCTTACAATG
CACGAGAATACCTTGCCCTCATTTATTTCGGTGATTTCAACCGTATA
TAAGTTATGAGGAAATTACAGTAGTTCAGAAGTTAAAATTTTC[C/T]
TGTAAACTTATGGAGGATTCATTCATATTGCAATATTTGCTGCTGACC
CAATATTGCTGCAATATTTGCAGCAGAACGCAAATCCCGAGAGCTC
ACTTTCC

Following is a MAPK10 genomic nucleotide sequence that includes the MAPK10 SNP “MAPK10-AJ” (SEQ ID NO: 4949).

GGTATCGACAAACACTGACAATCAGTACCATTTTAAAAGGTATCAATG
GTAAATGCCTTTCATTCTTGAGCAAATGTGACATTGCTTTCGGCATGA
AAAGACAGATGAAGTTTTAGGAGCTTTAAAAATGGTCTAAGAGAATA
GCAGTCAGCAAATCATTTTTTAGTAAATTGTTTTACAAGGAAAAGCCT
TGTTGATACTTTCTGAGTTCAATAAAATAAAAAGTATACTTTTTTAAGT
AGTAGACTTACCTTTCCATTCTTCAATTGTGTGTTCTCTTTCATCCAA
CTGCTTGTCATATATCTGAGGTGGAGGCTGCCGAATAAAACAAAAA
ATAATCTTTGTGTGATAATGTAAACATAACTAAACTCTTACAATGCAC
GAGAATACCTTGCCCTCATTTATTTCGGTGATTTCAACCGTATATAA
GTTATGAGGAAATTACAGTAGTTCAGAAGTTAAAATTTTCCTGTAAAC
TTA[T/C]GGAGGATTCATTCATATTGCAATATTTGCTGCTGACCCA
ATATTGCTGCAATATTTGCAGCAGAACGCAAATCCCGAGAGCTCAC
TTTCC

Following is a MAPK10 genomic nucleotide sequence that includes the MAPK10 SNP “MAPK10-AK” (SEQ ID NO: 4950).

TCTCCATTTCTGCCATAGCACTAGTGAGTGACTTTGAGTTGAAATACAA
TTTAAAAATAATACATTCTCTCTCTTTTTTCTTTCTCTCGTTTCCTCTGT
CCCTTCC[A/G]CTCTCTTTCAGATTATTAGTTTATTAAATGTCTTCAC
ACCCCAGAAAACGCTGGAGGAGTTCCAAGATGTGTAAGTAACAAAAC
TCATGAAGGAAGGGAGAGCACAGCAC

Following is a KIAA0861 genomic nucleotide sequence (SEQ ID NO: 3).

| >3: 184201001-184348700 | |
|---|---|
| 1 | aaagccaaga ctcccattcc taaaccctag ctcaggtctc catctcttaa atccgagtga |
| 61 | cctctacaaa ctctccctga gaaggtgtcc agaacccttt tggaagcgag ggacagtgtc |
| 121 | actgtctttg gggttgacac ctgctctgag taactcacgg aaaacaagtt ccagctggga |
| 181 | agcccttgga cgcgccacac ctccctaccc gcagcccgtc ctgtggcgcc cgggactcca |
| 241 | gagtgYgttt agatgaaagg ggaccgacac gtcagggcca ccgcgggaag cgctgagggc |
| 301 | cactcaccgg ccagggacgc gaagagcgcg gccgccgcgc tgagctgccg gggcatggtg |
| 361 | ggcgctgggc gaggttctgc agcgtgcggc gaRgtccggg caggccccga atcggtgcca |
| 421 | gagaaaccta cctgtgccgg agaaacgaaa ccacctgctt atgagaagca gccgaaaagc |
| 481 | ccgcccaggg ccgctgggcg gggagggaaa ctccgccggc cccctcctac ccctacggag |
| 541 | cagggagggg cggggactcg gcgcagccgc cggggcccgg gcctctggga ccgtttaccg |
| 601 | cacgcgcgtg gtcccggcag cgccggcStc ctccgctcat accctgggtc tcctcctttc |
| 661 | tttttctttt ctttttgaga cgaagtctcg ctctgtcgcc cagggtggag tgcagtggcg |
| 721 | cgatctcggc tcactacaac ctctgcctcc cgggttcaag cgattcttct gcctcagcct |
| 781 | cccgagtagc tgggattaca ggcatgcacc accacacccg gctaattttt gtatttttag |
| 841 | tagagacggg gtgtcaccat attggccagg ctggtctcga gctcctgacc tcgtgattcg |
| 901 | cccgcctcga cctcccaaag tgctgggatt atagacgtga gccaccgagc ccggccaggg |
| 961 | tctcctcttt tatttctttt ctttttattt cttttgtttt gttttgtttt gttttgtttt |
| 1021 | ttgagacaaa gtctcgctct gtcgccaggc tggagtgcag tggcgggatc tcggctcact |
| 1081 | gccctggttc aagcgattct cctgccgcag cctcccgagt agctggggtt acaggcgccc |
| 1141 | gccaccacgc ccagctaatt tttgtatttt agtagagacg gggtttcacc ctgttggcca |
| 1201 | ggctggtctc gatctcctga ccttgtgatc cgcccgcctc ggcctcccaa agtgttggga |
| 1261 | ttacaggcgt gagccactgc gcccggccca gggtctcctc ttttctaaca gctcgggtac |
| 1321 | ctttctggga acccagagac gcttctcagc cgggagaaag ccagccacta ggcgagcagg |
| 1381 | agcctaaaaa cccctaagca ccctgactcc atgtcttccc agggagtctg cggcagccgc |
| 1441 | gctccacgcc caggcctcgc caggaccgcg gtttgcggga agcaacagga gcacagccca |
| 1501 | gaggcgctag gtctggctgg gagctcgcgc tgccgactcc ccggcgtgcg gcgtcgggga |
| 1561 | acctctagga gccttggatt cttcagctgt aaaacggaca taataatgcc cactcccagt |
| 1621 | gtgttttttt attttctttt ttctttttct ttctttgttt ttgtttgttt gtttttgttt |
| 1681 | ttgtttttga gacagggtct cactctgtcg cccaggctgg agggcaatgg cgtgatctcg |
| 1741 | gctcactgca aacttgggtt caggcgattc tcctgcctca gcctccacag tagctgggat |
| 1801 | tacagatgtg cgccaccacg tccggctaat tttttgtatt tttagtagag accaggtttc |
| 1861 | accgtgttgg ccaagctggt ctcaaactcc tgaccccagg tgatccgccc gcctcggcct |
| 1921 | tccaaagatc tgggattaca agcgtgagcc actgtgcctg gccccaggtg gttttacaga |
| 1981 | ccagaaaatc ctggaacaaa aaacacacaa tatcgttttt tttttttttt tggagtcagg |
| 2041 | gtctcgctct atcacccagg ctggagtgca gtggcgtgat ctcggctcac tgcaacttcg |
| 2101 | acctcctggc ctcaagtgag tctcccacct tagcctcctg agtagctggg accaaaggcg |
| 2161 | cgtgccacca cgcccagcta ttttatttta ttttatgtag agaggaggtc tcgctgtgtt |
| 2221 | gcccaggctg gtctcgagtt cctggcctca aatgatcctc ctgcgttagc caaccattgg |
| 2281 | gattacaggc gtaagccacg gcccacggct caacaacgct gacaggcaac cttttaatgt |
| 2341 | cttatctcct tcctctatta attggattgt ctgtcaaaac aacgatgttt tgacagggct |
| 2401 | tgagtcccag tggggaatac acatttaagc agtatattag gagaccctcc ttatcactag |
| 2461 | attgagggct ttcagcctag cctcaaatta ttttctgaaa aataactttg gctacaacta |
| 2521 | ttttgtctta ctatgttgct ccaaacacta atcaagtaaa cttaaccaaa gcttgcagtg |
| 2581 | tgtttcagaa tggaattttt atggtgaaaa gtgagggtta acttgtgcca gtcaacctag |
| 2641 | tttcagcaac tacctgcttt ctgatctttg agacagttta ttcaaaagac gataattaag |
| 2701 | tgggtataga ctgtgtgcca ggcactcttc ttattccatt taagcgccat agccactcta |
| 2761 | tatggacact gttgttatta tcgctgcccc atttcgcaga tggagaaact aagcacaaag |
| 2821 | aagggagttg cccagagtca cttagataat aaataccgaa acctgaccat aaatcttgtc |
| 2881 | tgccttgaga gtctaggatt ttaagcacat agccgggcgc agtggctcac gcctgtaatc |
| 2941 | ccagcacttt aggaggccga ggcgggcgga tcacgaggtc aggagatgga gaccatcctg |
| 3001 | gctaacacag tgaaacccag ttctattaaa aatataaaaa aattagccag gcgtggtggc |
| 3061 | aggtgcatgt agtcccagct actcgggagg ctgaggcagg agaatggtgt gaacccagga |
| 3121 | ggtggagctt gcagtgagcg gagatcgcgc cactgcactc cagcccgggc gacagaagga |
| 3181 | gactccgtct caaaaaaaaa aaaataaata aaataaagct gctcctctta ccctggaaat |
| 3241 | tccaagggat ttaggagctc tgtttcagga accagggtca aagaccaagt attaaaacaa |
| 3301 | aagattctcc tagaactctg gcatataagg attttaggag ctctgtctta gaaactggga |
| 3361 | cagagaccaa agatatatta ttatatcgca gtatcatagt ttattatttt caaaaaacgt |
| 3421 | tttctggctg gtacagtggt tcatgcctat aatccaagca ctttgggagg ccaaggtggg |
| 3481 | agggtcactt gaggccagaa gttcaagtcc agcctgggca acacagggag accctgccac |
| 3541 | tattaaaaat tttttaaatt agctgggcat ggtggcacat gcctgtagtc ccagctactt |
| 3601 | gggaggctga agcaggagga ttgcttgagc ctgagaggtc aagactgtag tgagctgcga |
| 3661 | tcaagcgact gcactctagc ctgggtgaca aagccagacc ctgtgtctaa aaaaaagaaa |
| 3721 | agaagaggaa aaaaaaaggt ttttatttca actaagttgt tggatttatt agcataaagc |
| 3781 | ttttcataac aatcccttgt cttacaatat ctgtagtata ggtagtgatg tcacttcttt |
| 3841 | tattactaat attaataatt tgtattttct ctcttatttc cctgctaata ttaataattt |
| 3901 | gtattttctc tcttttttcc ctgataagtt tggctggagg ttgatcaatt ctattcatgt |
| 3961 | tttcaagaaa aaaaaaaaaa tttcatttca tcgagttcct tcattgttct tatgttttct |
| 4021 | gttttattca tttctacctg atctttatta ttttctttct tctacttaac ttgtgtttta |
| 4081 | tttgctcctc tttcaatagt tttcaaagat ggaacttgcc tgggatatcc cagcacttta |
| 4141 | ggaggctgag gtgggaggat cacctgaggt caggagttca agaccagcct ggccaacatg |
| 4201 | gtgaaactcc gtctctacta aaaatacaaa aattagctgg gtgtggtggt gggcacctgt |
| 4261 | gatcccagct actcgggagg ctgaggcagg agaatcgctt gaacccggga ggtggaggtt |
| 4321 | acagtgagct gagatcacgc cactgcactc cagcctgggt gacaggagct agactctgtc |
| 4381 | tcaaaaaaaa aaaaagaaaa aaaaaaagat ggaagttgag gccagtggta taaaatcttt |
| 4441 | cttcatctct aataaacagg tttactgtta tgaacgtccc tctaattact actttagttg |
| 4501 | gatcccacaa gtttaatatg ttttcatttt catataatta gaaagacttc ctaatttcct |
| 4561 | tttgctatct cctcgactca tggttattta aaatagcatt atttcatttc caaacatata |
| 4621 | gtttttcaga tatctttctt ttattgattt ttaattccac tgtggttgaa aacataatta |
| 4681 | tggacttaaa tcttacaaat ttattgatat ttgttttttg accaagacta tggttggtta |
| 4741 | cattagtttt caggactgac ataacaaagt gccacagact gggtaattaa accacagaca |
| 4801 | tttgctttct tataattctg gaaaccagac atgtgagatc aaagtgtcag ccgggttggt |
| 4861 | tttttctttt tccttttttt tttttttttt tgagacagag tctctttctg tcacccaggc |
| 4921 | tgggatgcag tggtgtgatc tcggcttact gcaaattctg cctcccaggc tcaagcgatt |
| 4981 | ctcctgcctc agcctcccga gtagctggga ttacaggtgc ctctcactgc acctggctaa |
| 5041 | tttttgtatt tttagtagag acggggtttc accatgttgg ccaggttgat ctcaaactcc |
| 5101 | tgatctcagg taatacaccc gcctcggcct cccaaagtac tgagattaca ggcgtgagcc |
| 5161 | actgcacccg gcccgggtta gttctttcta aggcctctct ccttggctag tagacacctt |
| 5221 | tgtttcacat ggtcatccct ctgtgcatgc ctttgtctgt cctaatctcc tcttcttata |
| 5281 | aggacattag tcaggtagga ttagtgccta ctctttgaac tcgttttacc tcttaaagac |
| 5341 | cctatctccg aatatagtca cattctgaga tacttggggt taagacttgt attagtccat |
| 5401 | tttcacgctg ctcataaaga catacctgag actgggaaga aaaagaggtt taattggaca |
| 5461 | attccacatg gctggggagg cctcagaatc atggtgggag gcgaaaggga ctttttacat |
| 5521 | ggtggcggca agagaaaatg aggaagaagc aaaagcagaa acccctgata gataagccca |
| 5581 | ccagatatca tgagatttat tcactgtcat gagaacagca cgggaaagac cagcccccat |
| 5641 | gaatacatta cctcttcctt ggtccccccc tccccacaat atgtggggat tctgggagat |
| 5701 | acaattaaaa ttgagatttg agtggggaca cagccaaacc atatcattct gtccctggtc |
| 5761 | cttccaaatc tcatgtcctc acatttcaaa accaatcatg cctttccaat agtccctcaa |
| 5821 | agtcttaact catttcagca ttaacctaaa agtccacagt ccaaagtctc atctgagaca |
| 5881 | aggccttccg cctatgagcc tgtacaatca aaagcaagct agttagttcc tagatacaat |
| 5941 | gggggtacag gtattgggta aataaagcca ttccaaatgg gagaaattgg ccaaaacaaa |
| 6001 | ggggttacag ggcccatgca agtctgaaat ccagtgaggg agtcaaattt taaagctcca |
| 6061 | aaatgatctc ctttgactcc aggtcttaca tccaggtcac gctaatgcaa aaggtaggtt |
| 6121 | tccatggtct tgggcagctc cacccctgtg gctttgcagg gtacagcctc cctccaggct |
| 6181 | gctttcatgg gctggtgttg agtgtctgca gcttttccag gcacccagtg caagctgtca |
| 6241 | gtggatctac cattctgggg tttggaggac aaaggccctc ttctcacagc tgcactaggc |
| 6301 | agtgccccga tagggactct gtgtgggggc tctgatccca catttccctt ctgcactgcc |
| 6361 | ctaagaggtt ctccttgagg gccccacagc ttccaccctc tgaaccatag cccaagctat |
| 6421 | gcattggccc ctttcagcca tggctggagc agctgggaca gagggcacca agtcactagg |
| 6481 | ctgcacacaa catggggacc ctgggcctgc cccacaaaac ccctttttcc tcctgggcct |
| 6541 | ccaagcctgt gatgggagag gctgctgtga aggtctctga catggccttg gagacatttc |
| 6601 | cccatggtct tggggattca cattaggctt cttgctactt atgcaaattt ctgcaaccag |
| 6661 | cttgaatttc tccccagaaa atgggttttt cttttctgtc acatagtccg gctgcaaatt |
| 6721 | ttccaaactt ttatgctctg cttcccttat aaaactgaac gcctttaata gcacccaaat |
| 6781 | cacctcttga atgttttgct gcttagaaat tttttccacc agatacccta aataatctct |
| 6841 | caagttcaaa gttccacaag tctctagggc aggggcaaaa tgtggccagt ctctttgcta |
| 6901 | aaacataaca agaggcacct ttgctccagt tcccaaaaag ttcctcatct ccatctgaga |
| 6961 | ccacctcagc ctggatctta ttgtccatat cactatcagc attttgggca aaaccattca |
| 7021 | acaagtctct aggaagttcc aaactttccc acattttcct gtcttcttct gagcccttca |
| 7081 | aactgttcca atctctgcct gttacccagt tccaaagttg ttccacattt tcaggtatct |
| 7141 | tcagcaacgt ttcactctac tggtagcaat ttactgtatt agtccatttt cacactgctg |
| 7201 | ataaagacat atctgagact gggaagaaaa ataggtttaa ttggacttac agttccacat |
| 7261 | ggctggggag gcctcagaat catggtgaga ggtgaaaggc acttcttacg tggtagtgac |
| 7321 | aagagaaaat gaggaagaag caaaagcgga aacccctgat aaatccatca gatctcatga |
| 7381 | gacttattca ctatcacgag aatagcatgg gaaagaccgg cccccatgat tcaattacct |
| 7441 | ccccctgggt ccctcccaca acacatggga attctgggag atacaattca agttgagatt |
| 7501 | tgggtgggga cacagccaaa ccatatcaag acttctacat atgaattttg gagggacaca |
| 7561 | atttaactca taatagtgga ctgtcYtgtt aaatgttctg tgtgcacttg agaagaatgt |
| 7621 | gtgtattctc tcattgttgg attcagtgac ctataaatgt taattaggtt aaactaattg |
| 7681 | atgtagggaa aagaaagaga gatcagactg tcactgtgtc tatgtagaaa gggaagacat |
| 7741 | aagagactcc attttgaaaa agacctgtac ttcaaacaat tgctttgctg agatgttaat |
| 7801 | ttgtagcttt gccccagcca ctttgcccca gccactttga cccaacttgg agctcacaaa |
| 7861 | aacatgtgtt gtataaaatc aaggtttaag ggatctaggg ctgtgcagga cgtgccttgt |
| 7921 | taacaaaatg tttacaagca gtatacttgg tcaaagtcat cgccattctc tagtctcaat |
| 7981 | aaaccagggg cacaatgcac tgcggaaagc tgcagggagc cctgcccttg gaagcggggt |
| 8041 | attgtccaag gtttctcccc atgtgacagt ctgaaatatg gcctcgtagg atgagaaaga |
| 8101 | cctgactgtc ccccagccca acacccataa agggtctgtg ctgaggtgga ttggtaaaag |
| 8161 | aggaaagcct cttacagttg agatagagga aggccactgt ctcctgcctg cccctgggaa |
| 8221 | ctgaatgtct tggtgtaaaa cccgattgta catttgttca actctgaaat aggagaaaag |
| 8281 | ctgccctgtg gtgggaggtg agacatgttt gcagtaatgc tgccttgtta ttctttactc |
| 8341 | cactgagatg tttgggtgga gagaaacata aatctggcct atgtgcacat ccaggcatag |
| 8401 | taccttccct tgaacttaat tatgatacag attcttttgc tcacatgttt tttgttgacc |
| 8461 | ttctccttat tatcaccctg ctgtcctact acattccttt ttgctgaaat aatgaaaata |
| 8521 | ataatcaata aaaactgagg gaactcagag gctggtgccg gtacaggtcc ttggtgtgct |
| 8581 | gagtgccggt cccctggact cactgttgtt tctttatact ttgtctctgt gtcttatttc |
| 8641 | ttttctccgg ctctcatccc acccgactag aaatacccac aggtgtggag gggcaggcca |
| 8701 | ccccttcaat tgatagtatg gttcaagaac aaatggtatc aacttaggat ggtttaactt |
| 8761 | atgatttttc aactttagaa tggtgtgaag tctgtatgca ttcagtagaa ggcatacttt |
| 8821 | gaatttttat cttttcccaa gctactgcta tgggacaaga tactctctca caattctggg |
| 8881 | cagtggcagc aagcctcagc ttccagtcag cacccaatcc caagggtaaa caactgatac |
| 8941 | agccattctg tttttcattt ttagcaaaat actcaataaa ttacatgagg cactcaatgc |
| 9001 | tttattataa gacaagcttt gtattagatg atttgcccaa ctgtaggcta atgtaggtgt |
| 9061 | tctgagcaca tttaaggtag actatactat gccatgatgt ttggaaggtt aggtaaattt |
| 9121 | aatgcatttt cgacttagaa tactttcagc ctcccaaata gctgggacca caggtgtgtg |
| 9181 | gcaccatgtg tggctaattt tttgtggaga caaggtctca ctRtgtcgcc caagttggtc |
| 9241 | tcaaaatcct gacttcacgt gatccttcca ctctggtctc ccaaagtgcg attacaggtg |
| 9301 | tgagtcacca cacctggccg tgttgtcaca cattttaatt ctttataaat tagaaacttc |
| 9361 | acaacacatt gttcttattt aaaatttaaa caattatctt taaatataca taatatataa |
| 9421 | atatgtatat aatatataaa tatatacaat atataaatat ataagatata ttatatatat |
| 9481 | aatatataaa tatataatat ataaatatat aatatgtaaa tatataatat ataaatatat |
| 9541 | gtaaatatgt aatatgtaaa tatgtaatat gtaaatatat aatatgtaaa tatataatat |
| 9601 | ataaatatac ataatatata aatatataat atataaatat ataatatata aatatataat |
| 9661 | atataaatat ataatatata aatatatatt atatataaat atataatata cataaatata |
| 9721 | tataatatat aaatatatat aatatacata aatatataat atataaatat atataatata |
| 9781 | tataaatata tatggggaaa aaagctttta tacttactca tgtgattacc gtttcttgca |
| 9841 | gtctttattc ctttgtttag atccttggga tttttgttgc tatggtgacc ttagttatac |
| 9901 | caggcacttc aaatcttacc ttgtgtttag gaWatgggct tgtttgccca aaggtcttcc |
| 9961 | ctaatgtctg ctccaccttc aactttaggt cttctctgct gtcagtttct ctctccatac |
| 10021 | acttgtagct ctcccaggag tattccatcg ttacttgtta gtcagtgctt attagcgggg |
| 10081 | tggtgggatc tgagaggtga ggtgcttggt tgtgattctc agttctgatt cctgcaggta |
| 10141 | ctgtgtccct gggYcttgaa gggtatgacc aagcStctct gtccctcccc gcagcagtag |
| 10201 | ttttgggctc agcacatatt cctgcccctt ccccagaatc agagggtttt ttgttgttgt |
| 10261 | tgttttgttt ttcaagtttt tgttcctttt tctccatctg tgttggattt accagccccc |
| 10321 | taggagcgtc agtatttgtt acccttcctc caggctttta aggcctttgt aggagagatg |
| 10381 | ggccaatagc atctgagcat ggttttgtgt ctttcttgta gcaactgata ttcctcaccc |
| 10441 | ccaggtctat gccaggaagg atgcttcctt acatgcctgt aatcccagct accggggagg |
| 10501 | ctgaagcaga agaatcactt gaacccagga ggcagaggtt gcagtgagcc aagatcatgc |
| 10561 | cactgcactc cagcctggca acagagcgag acttcattca aaaaaaaaaa aagctcttac |
| 10621 | actcttctgc atattctgca aagtatactc tgggaaggct ggtttagagg atctctaact |
| 10681 | tttctaatat ttcataatgc atggctgatg tttaacatga actcagactc ccatcagatt |
| 10741 | ttacagatct gggtctgctt ttagttctaa tgcttcagcc agggcacttt aatctgaggt |
| 10801 | cacagcattc ccaattgtca gcagtatttt gaaatctaag ttcctctggt ggacaacagg |
| 10861 | caacatcata tagccaYgga ggatctgaac aacttctaag tctgagSgat tttatctggt |
| 10921 | ggttttggat tcttttccat ttatacttaa acaaggtgac acaaatgtca ctcacagaaa |
| 10981 | agccactctt ttttcccctg tttttaagaa gtctaaaaat gcacaactat ttcctgaatg |
| 11041 | agcagtgtgt ccaggtgtag aatagggaat gtcgtgtcca ctgggaacag acggtgtacc |
| 11101 | tggcacaatt ttatgagaat tatcccatga ttcctgtccc accctgcagc ccaccatggg |
| 11161 | ctcagtgggg agtctgagtc tctgagcccc aaagggtagc tttttcccag acgacaccag |
| 11221 | caagcagagc agcagcttgg ggctttgaca aggaggctgg agtgaaaggt catattggac |
| 11281 | catcctggRt cactggagga caatacccca ggaacctcca gcaatgactg gtattggaat |
| 11341 | gactcccctg gtcacccacc tgtggcaaga catttgcaaa cagggtcttc ctatgaggct |
| 11401 | tgtgaggttt ggccaatttg cattctgcta gaattattaa gtatggacat cctcatagat |
| 11461 | ccagaaaagg gccgagatcc aaaggattga gaatgctttg gggggtgttt ccatagtgag |
| 11521 | tggctggcta ttcaacagtc aaatgtttga gaggatacaa gtgatgtgtt tcctgaggca |
| 11581 | agtggtcaga acccaacaga ctcttctgtt cagctaagat gagagaccat ctgaagttct |
| 11641 | tacatgttct cacagagaaa tggattctca tagggaaatg gatatattgt gatagacact |
| 11701 | gtaagcagag aagttgactg agaaacacac acacacacac acacacacac acacacgtca |
| 11761 | agactgaatg catagatgtg tattataaaa agYgtaaaaa tacaccacca ttStaacaga |
| 11821 | ttgtgtagga ctatattttc ttttttttta atttattatt attatacttt aagttttagg |
| 11881 | gtacatgtgc acaatgtgca ggttagttac atatgtatac atgtgccatg ctggtgtgct |
| 11941 | gcacccatta actcgtcatt tagcattagg tatatctcct aatgctatcc ctcccccctt |
| 12001 | cccccacccc acaacagtcc ccagaatggg atgttcccct tcctgtgtcc atgtgttctc |
| 12061 | attgttcaat tcccacctat gagtgagaat atgtggtgtt ttgttttttg tccttgcaat |
| 12121 | agtttactga gaatgatgat ttccaatttc atccatgtcc ctacaaagga catgaactca |
| 12181 | ccatttttta tggctgcata gtattccatg gtgtatatgt gccacatttt cttaatccag |
| 12241 | tctatcattg ttggacattt gggttggttc caagtctttg ctattgtgaa tagtgccaca |
| 12301 | ataaacttac gtgtgcatgt gtctttatag cagcatgatt tatagtcctt tgggtatata |
| 12361 | cccagtaatg ggatggctgg gtcaaatggt atttctagtt ctagatccct gaggaatcgc |
| 12421 | cacactgact tccacaatgg ttgaactagt ttacagtccc accaacagtg taaaagtgtt |
| 12481 | cctatttctc cacatcctct ccagcacctg ttgtttcctg actttttaat gattgccatt |
| 12541 | ctaactggtg tgagatggta tctcactgtg gttttgattt gcatttctct gatggccagt |
| 12601 | gatgatgagc attttttcat gtgttttttg gctgcatcag tgctctacac gttcagagaa |
| 12661 | acttctctag tgacgaacta tagaaatgat ccctgaaagt atagtcttag gactatattt |
| 12721 | tcttttgact tgggaggcat gtttattgct gttaatgctg caaagggctc tacgtgcttt |
| 12781 | aaaaaatccc aatctgttgc attcataagc ctgggttgga tctaaagcag cctcccactt |
| 12841 | ttggaaaggc atccccacga cctttccatg gttgctgaat gcagctggag gcagtcacag |
| 12901 | ctggtgatgt ccggagccca ttccccactg tgctggtctg cagaacttct gcatgccatt |
| 12961 | cccacaagca ggtctctgcc ctgctctcct ccacctccct tgtcagagga agtctgcact |
| 13021 | tcacagcttt ctggtctcaa ccctcctcca tccctacaga tgtgtaagca gcaggaatca |
| 13081 | aaaggtgaag gagagggggc aactcacctc cgatggacac gtgaaaagtg ggagatggat |
| 13141 | aaaatcaaga aggagcttaa gatatccaga aatgtaaact gtgtttggaa aagtaaggtc |
| 13201 | aggagaagca tgggactcct gaggttgctc cctactatct tgcagacttg ctgcaggacc |
| 13261 | aaatgaagca ggatctgtca agcaccaggg ccagctctta agcttagtgc ctttctgaac |
| 13321 | cctgtgaccc agcagcctcc atcaactcgt cctacctgcc atgcacagct cctctgtgcc |
| 13381 | cctgtacctg agctcatgct attccctctg ccaggatgcc cttctccttc tccaccagga |
| 13441 | gaagaacact tgccagtaag acccagttct aatgtcaccc cttcctgacg gtatcaggaa |
| 13501 | gagtcaStga tggtgtttta tgctcccaga gaatttgcca cattgtgttg tgattatttt |
| 13561 | tccacatctg tctcccccac tggaatgaga gcctcactca tcttcatacc tccctggtct |
| 13621 | ctacctggtg ccagaaccat cctcagggca ggggaatgct caggaaatag atattgaata |
| 13681 | aaataagtgt atccatccat ccatccatcc atccttccat ccatccagac atatatacat |
| 13741 | atgtaaacat tctgatggtc aaatggaaca atgtgggctg aagaataatg caggtagaag |
| 13801 | aacctaagat tacagattct tgatttggga ggaactttat tttattgtgc aaacagtcta |
| 13861 | caaatttgaa acatactcta tcaagaaaga cactgttgtt aggaaagggc gtgggagagg |
| 13921 | gtggccccac aaaacagtgt aaagttctaa agaatttgag aggaacactc tgcgggaccc |
| 13981 | tgttccaagg gcatctttct aagagtctgt cccttaggct ctgccccttt ttggccacgt |
| 14041 | tgtcaaaggc cttctttatg aacgaagtga gacacattat ttttgttttc ttgaattcta |
| 14101 | aagtgattgt cttgagttcc ctgggggaga ctcctgggag gtgtggccca ctctattcca |
| 14161 | gaaacaaacc aggagactga agactcatcc caatcccata tcccatgaaa atgtgaaatc |
| 14221 | aaatcacccc tgacaattcc aaaactaaYg gaatcctaac ataactctat gtaactctaa |
| 14281 | agctgagtgt gctggtaatt aagcttccag cccacccttc ccagctctgg ggtgcagggt |
| 14341 | caccatgggc tccttcctgt gtgcacccca ccccaccccc actgtcagca tcctgtctcc |
| 14401 | accctgagag tgacagctgg ttccagtagc gggagttggt tccagcttgc cattttccta |
| 14461 | gcactcctat aaccagcctg ctgatgccca ttcagagaca gcagcacggg ctggccatgt |
| 14521 | cccctctcca gaattctgcg tccagctcct ggaccttgag ctctgagccc ttgggccacg |
| 14581 | tgtacMatta atagtgcctc ctcctcagag gactaacccc cagccctagg gccaccttca |
| 14641 | tatttcYgag ttttgatatt ttcaacctct tttctttgtt gtatgagtcc ttgggctggg |
| 14701 | agcttgcagt caaaatcttc atgatatctc attatcacta ctttttttta aatctctact |
| 14761 | agctggataa caattattta tattaaattc tctcttgaaa taactgatac agtgtctctt |
| 14821 | gattgaaact tgactagtag actaagaatt ctaactctaa ataattctga gggccgggtg |
| 14881 | tggtggctca cacctgtaat cccagcactt tgggaggcca acgcgaatgg atcacctgag |
| 14941 | gtcaggagtt ggagaccagc ctggcaaaca tggcgaaacc cggtcaggag atccagacca |
| 15001 | tccYggctaa catggtgaaa cctcatcttt acttaaaaaa aaaaaaaaat acaaaacaaa |
| 15061 | attagccagg cgtggtggca gatgcctgta gtcccagcta ttcaggaggc tgagacagga |
| 15121 | gaatagcgtg aacccgggag gtggagcttg cagtgagccg agattgcacc actgcactcc |
| 15181 | agcctgggtg acagagagag actccgtctc aacaacaaca acaacaaatt agccgggcat |
| 15241 | ggtggcaggt gcctgtaatc tcaactactt gggaggctga ggtaggagaa ttgcttgaac |
| 15301 | ccgggaggcg gaggttgcag tgagccaaga ttgcggcact tcactccagc ctgggtgaca |
| 15361 | agagcaaaac tccatctcaa aaaaaaaaaa aaagaaaaaa aaatctgaca attaaataaa |
| 15421 | gaacagaaaa aaaatttgaa tggcaaatac aaagctgaaa agaaataact gagaataaat |
| 15481 | aactctgaaa atagctcaaa aactaaatac ctcaaaaact ctttaaaaat tcagaaaata |
| 15541 | taatgttcaa atatgaagta atgctgaaaa tgaaataact aaaaaccaag taacttgaaa |
| 15601 | aaaagaacat aaaaataaac aactcaaata taaaataact gaaaataaat acctgtgaac |
| 15661 | ataagcaact cgaaaaccag ataactaggg gaaacccttc attaaaacat ttcactctga |
| 15721 | aaataaataa cttgacagta gttcatgaac ttccagtgag tgtttaatag tcaaataagt |
| 15781 | tactgtaaaa ataaataact caaaaactcc aataagataa agtgaaataa ctatgaaggt |
| 15841 | aaataactca gataataatt gtaaagataa ttaaaaataa attccggctg ggcgcggtgg |
| 15901 | ctcacgcctg taatcccagc actttgggag gccgaggcag ggggatcacg aggtcaggag |
| 15961 | atcgagacca tcctggctaa cacggggaaa ccccgtctct actaaaaatc caaataaaaa |
| 16021 | attagccggg cgtggtggcg ggcgcctgta gtcccagcta ctcaggaggc tgaggcagga |
| 16081 | gaatggcgtg aacccgggag gcggtttgca gtgagccgag atcacaccac tgcactccag |
| 16141 | cctgggcgac agagcgagac tccgtctcga aaaataaata aataaataaa taaataaatt |
| 16201 | ccaagcaaat taatttgaac atctgtaact cctaaaacaa aacaactaca tataaataac |
| 16261 | tgaaaatgaa caactaaata actttgaaaa taaagtaact ataaaccagt aactgaaact |
| 16321 | ataaaattaa ataaccagaa agctacaaag aaatgaaaat caactaataa aactaaaaat |
| 16381 | aaaatacaac tgaaactaac aaaactcatt taaaaactaa taaatcgata cataaaatac |
| 16441 | ctcagaaagt aacttggaac gaactctctc tacaacgaag caatcttggc actaacacga |
| 16501 | acaccccatg aacgctcgca gggatgctgg gaggcggacc gggagctccc agtctgcggc |
| 16561 | ccccgccggg ttKgagcggc tccggctcct ccaaggctcg ggctaagcgg ctctcaaccg |
| 16621 | attcccaccc cgccctggag aaatgcgggc gtgtctgcag gcatttttga ttgtcacgat |
| 16681 | ttgggggcgg ggtggaggat ggggtggcgc attcctggca cctagtgggt agaggccagg |
| 16741 | ggtgctacta aacatcctac cgtgggagca cagagaagcc cacgcagcaa agaattgccc |
| 16801 | ggctccgaat Rtcgaagtgc gcggtcgaga aggcgtgggc tgcgggctct gctcgcctct |
| 16861 | gcaggcgcct tagagcagct ccgaggtccc ccgtgcggag ctaggcgcgc acccaggaca |
| 16921 | cccctcgggc tcctcggagg aggccctggt tgtccccttt ctgccgccgc cgaggctccg |
| 16981 | gctgctttct gcgtagctgg gcagggcccg ggcccccaca ccgcctctcc cgggaatgcg |
| 17041 | ggcgctctgg agccgaggag cgggggcgtc cgcagggagg tcagctctcc tgggcggagg |
| 17101 | tcctcgggcg cagcgccctc gcctggaaac cagccgtcgc ccccgcagga gccagccggc |
| 17161 | ccgtggacgc cccagcgcgc tcctcctcgg tgctgcgggt cgccctgcaa ttccgagaag |
| 17221 | aaagtcagag acgccgtggc ccaaagaggc gcttagtctt tcctcgctca cactcacgtt |
| 17281 | tcctcctcat cgcgttcttc tttttctccc tggctgcttt ctcccctctc caggaaagca |
| 17341 | gatttggagg aacaggtttc gtgactgtcg tccgactgga aaaggcccgc gagctggaag |
| 17401 | ggaggggacg ggtgcaccct cagagttatt gctggaggct gtggccagac cgggcaaggt |
| 17461 | ggtgactccc gctggcaggc tgaggcccac cccagccctc ccacctgggc cacggggctc |
| 17521 | tcagcgggag ccccagttat gaccggacac cagcgcaccg ccaaggagac agccacgtgg |
| 17581 | ggacatgctg gactaggagg gtcagagcca gtttgagggt ctggtgacct cggctccctg |
| 17641 | gcttaaccag gtccttatgg gtgagaatcc tgaggagggg gagagggatg gaggctaggg |
| 17701 | acaggaggca ggaggagctg atgaatagga aggagggaag aagatgaaag aaacaaaagg |
| 17761 | gaagtaatta cactcagagc actgctctcc ttttccatgt cttctgcgcc ttcacagtct |
| 17821 | tcaaaggtgt ccatggagct aaactccctg ctggagcagc cttagggaga gaaagggaag |
| 17881 | gatggggcct ctgtggggtg ggaggacatc ccctctgccc atggcagggt gtagcaggca |
| 17941 | gtgcctgttg caggcacggt cctccccatc tctaactcct gctctccaag ggcctgcact |
| 18001 | gcgctgggct gtgagggggt ctgtgatctc caggctgctt ttccagcgcc gagatgccgt |
| 18061 | aattcaccga gaaggcgcgt ccacatgctg ttcatggccc tacctccccg ttcctccaag |
| 18121 | aaaacagtca ttgttttttg tgtttgccag tcttctaacc acgcctcctt cccttcctcc |
| 18181 | tcccctgtct ctttctcacc ctcccctcct tgcctccttt tctctcccct aattaatgtc |
| 18241 | catttcccat ctccctggca gcctctgcca agtgtcactg ctccccataa gggaaaatca |
| 18301 | gaggaacaag caagtgcatc catcctgcct ctctctgcag tgaactgatt aattaatcca |
| 18361 | tcagtcttgt ctatggcgca catgttacat ccctggggcg gtgttggaca ctgtggggaa |
| 18421 | cagcagccac tgccaaatac tgaacaactg ccctgtgcct ggtggtatgt taggcatttg |
| 18481 | tcaaagttta agcctcacaa ccctgtaagg gtctcagccc cctttacagt tggggaaaca |
| 18541 | gacagcaatg gtcacttggc caagtcctct tggcctgtgg cagggcagct gtcttctcca |
| 18601 | gcactcctgc tctttaccct cgctctgagt gagatggagt ctctgcccca catggctcac |
| 18661 | aagccagggt agggagcaga gcatctgcag aaaggtccca acacaggaca gccccagacc |
| 18721 | gagggtcagc tgagtagtct acgcggcggg agccgtgcta ggaaagtgtc tgctcaggcg |
| 18781 | agaattcagg gaggtttggg caggacttga ggggcagaca ggactggaga gggagggcat |
| 18841 | tctgggcaga ggcatggcca gagggcggta ggcggcagtg gagggagctg cagttatctg |
| 18901 | ggtgagcagg cagcacaagt Rgcgtctcct gggctgctgc cccaagcccc caacaagcca |
| 18961 | cgttctgggc cccaggccct ccccagagca gatcagtggg ggctgtgtga gtaacatggg |
| 19021 | ggcggggggg cagctgggca gcacctccct ggaggcccct ctgaaatcct gcctgactct |
| 19081 | ggcaggctcc gagggggctg gacaccctcc tctcaggttg aagcaagtcc tggttgagtt |
| 19141 | cctagtccca ggaggtggga ggggcaaggg gtggagggca gaggagaaac tgcctcaggg |
| 19201 | atgtgccccc tgccttcatc ctccagacag gacttgggag catctaagga aacccaagac |
| 19261 | tcctctttag agaagtcatc cagccctggg gtccccttat gccaggagca agcagtgaga |
| 19321 | atggaagaat gattgtcttg ctgaaagttc tgtgatggag ggatagaggg acagagggag |
| 19381 | ccatgccctt gaccatcccc tgcatgaata ggaagggctg tgtctccagg gtccatggcc |
| 19441 | tctgtgcccc ggatgatgcc agggctgcta gggaccatag agccacccac tgggaggctg |
| 19501 | gcggttgggc ctggctcagg agccttcgtc agccataggc agccacagcc tggggtgggc |
| 19561 | agggctggga ggcgacacag gaactgaaaa acctgacaag ctctagcccc tccgcagggt |
| 19621 | aagtggtacc tccaggtaaa atgattagtt Kgttccagcc cctctgcagg gtaagtggca |
| 19681 | cctggggtaa aatgactgcc tggagctggc agctgctttc cctgctctcg cgggccctgc |
| 19741 | agggaagcgg ggaagggaag ggggcacagc gctgggcaca gaggggctct cagaccctgg |
| 19801 | actcaactgt ttcagggtca tctgaaacag tcaactgttt cctctagccc attccctgcc |
| 19861 | tccaggcgag gatttgcctg aacgtggaaa gaggaaggat cctcccagtg ctgtcaaccc |
| 19921 | cagattccac ctccctgtgg gggactgtca gcgcaggccc tgacaacgca gagaaagaca |
| 19981 | caggacccac ctgggccagt gacagcagga gctccgggtg ccacaggtga gggtggggat |
| 20041 | gcctggagca ccacgggggg cctggtttag tctagagcca ggttttccat acaccttaga |
| 20101 | gtgcaacctc agggagatgc aaattttacc ccctaacaca gcatacacgc agaaacacat |
| 20161 | ttatacaatt caaacacaag cggacggaac aatatttacc cttagagtgt gtgaagtcct |
| 20221 | catctgtccc acctcatcct atcatgcttt gttctatcct aggagacaaa gcaggagggg |
| 20281 | gggctgcgga ggtgggggag tctcatccaa gcccttgggt gacacgtctc tcctgagaca |
| 20341 | aactgcagct gctctgggtg tgccctcgcc tgtctccctc caggccccgg gttcctgcca |
| 20401 | gcagagacag taacctattc accaggtatc ccccagggct cctggaagaa actcagaatt |
| 20461 | ctcagaacca gaaaacctta gagagcatcc tgcaggcaaa gcccctggtt ctctgcggag |
| 20521 | gaaagtgcgg ctcacagggt gccccgccag ggatggtaat tgacYaccag gctgtgtgcc |
| 20581 | ttgtggggac tggcttaagg ccctgtggga gctgagtcag ggccaggacc ggggtgtcct |
| 20641 | gactcctaga gatcatgttc ccttcctcac ccaggccttc cagtcccagc cctgggcttt |
| 20701 | tatttattta ttttggagac agagtctggc ttgtcaccca ggttggagtg cagttgtgtg |
| 20761 | atcacggctc actgcagcct tgacttcctg ggttgaactg atcctcccac atcagcctcc |
| 20821 | tgagtagctg ggaccacagg cacatgccac cacacccagc taatttttgt attttttggt |
| 20881 | agagatgggg ttttaccatg ttggccaggc tggtcttgaa ctcctgagct caactgatct |
| 20941 | gcccacctca gcctcccaca gtgctgggat tacagatgtg agccatcatg cccacctcct |
| 21001 | gggctgactt ttgctgtctt acatcatctg catatttaat cccctgctgg attcactggt |
| 21061 | catgggctct gaggccctaa gagtcttagg cactaaggag ctggcagcac tgaggggacc |
| 21121 | ccaaaatctc agactcagga tctggccagt cacaggcatg tgagggaaca actgagaggc |
| 21181 | ccattgcccc atggcaggag aaggtgctct ggagtcagtc agacctgagg gcagtcagac |
| 21241 | ctgattctca ctctgtcact cactagctgt gtgatcttgg atacatcact taacctcttg |
| 21301 | agcatcagct tccttatctc taaaatggag ataataacat cgattttgca gtcttggtat |
| 21361 | gaggattagc aaatcttctg ataaagaaaa atgcctggta catcatagga attcaacaaa |
| 21421 | tagtacctgt tatgattatt gtgtatagca attacaataa tactaaagag agggtctcaa |
| 21481 | aacagctctg ggcactccag gtgtgctatt attacttaca tttcagggag gtttgtctgc |
| 21541 | cattgtctca tcctcataaa cactcaggga aagaaacatt ataaggataa taaatggctt |
| 21601 | taaaaagaaa cagagcaaac acacacacac acacacccct cagaaaaacc atgccaaaca |
| 21661 | cacaggctct tgacaaatat tcaatctgat tatagcaaaa ctgttttgtt ttgttttgtt |
| 21721 | tttttgtttg tttttgagac aggggtctcg ctctgtcgcc caggctggag tgcagtggcg |
| 21781 | tgatcttggc tcacagcaac ctccttctcc cgggttcaag caattctcct gtctcagcct |
| 21841 | ccagagtagc tgggactaca ggcacatgcc actatgcctg gctaattttt gtatttttag |
| 21901 | tagagacagg gtttcaccat attggtcagg ctggtctcga actcttgatc tcaggtgatc |
| 21961 | cacctgcctt ggcctcccaa agtgtgagat tacaggcgtg agccaccatg cccagctgat |
| 22021 | tatagcaaaa ttctaagtga tagttgtatt cttggaaaat gaatggaacg acttttgtcc |
| 22081 | cagccaagat ctagtggtgt gttggagcag atacaccctg agtctctggg gcactctcag |
| 22141 | tctatgtatc agataagcat aaggagtatt ggtggagaag gacaagaatg ggaaaggtgg |
| 22201 | gcgatgagaa ttcctgcaga taaggactgg tgagagtatt ctcttttgaa acccttagtc |
| 22261 | gacaatcttt ctggtgcata tcagataagc tgaatggttt aggaaatcta gtgttcacat |
| 22321 | ttagtgctta gaattctaag cttttttatt ttgcttaaac aaatggaatg aaatttatta |
| 22381 | acaagtgaac ctagtaatga gctgaaatta ttctcaccag catacatatt tttggtaaat |
| 22441 | tatagacttt gaagacaaaa tcatggtgtt tcccttactg tccagtggat ggcacaaaga |
| 22501 | gaccattgta gatcctgctg gttcagcggt agctctcaat ccatagatat taaatgggca |
| 22561 | aattctattt ttattgtctt tcaaactaga tttttctcaa tgcacaactt tttttttctt |
| 22621 | tttctttttt ttttttgaga cggagtctcg ctctattgcc aggctggagt gcagtgacac |
| 22681 | gatctcggct cactgcaacc tccgcctccc gggttcaagt gattctcctg cctcagcctc |
| 22741 | ctgagtagct ggggactaca ggcgcatgcc accatgccca gctaattttt ttgtaatatc |
| 22801 | agtagagaca gggtttcacc atgttggcca ggatggtctc gatctcctga ccacgtgatc |
| 22861 | cgcccacctt ggccttccaa agtgctggga ttacaggcgt aagccaccgc gcttggccaa |
| 22921 | ttatgcacaa ctttttaagg accatctcta tcacatgaac taaaggatat cattttcact |
| 22981 | tgggggtggg aagtggtgag ctgcttaaaa gcaatgccta aaccccctgg gctttccttc |
| 23041 | ctttcacttg gaagaaccag ggggtaacta actgctaaca attccatgct ttcagagatc |
| 23101 | taggcgcaag cctaagggct tcatctcatt taatcctcat ggcaagactg tacagtatta |
| 23161 | tctccatttt gtaaaatgaa tgataaaaag aacttaagca cagacaggct atagaatcca |
| 23221 | tccaaagaga tggagctgca cttgaggctg ggtctttgag acctgagttt gagcccttca |
| 23281 | tcattgtgtt gtgtgtgctg ctaaccaatt tcccctctct gtcttctagg atttaataca |
| 23341 | tggtgtcaca ttctgtctga tccatcccag tagctctcca gagctcccaa caggagagag |
| 23401 | ttccaaaatg tttccagggg tactaggctc ggttacaata ttttgcttgg tggcccagag |
| 23461 | tataaacgtg gatatcttag gctggtctat agctgaaacg tctatttcat ttgcaagcct |
| 23521 | atctttggct aagaggaagt gaatcattct tgagaacatc taattaattg ctttcagatt |
| 23581 | ccacatgttg acattctcag ggcacatttt ttttttcccg tgcatttctg ttgtcaaagt |
| 23641 | ccctgccagc tcctaaggca gtctgagctg gctgtcttag actttcagag ctgctggaag |
| 23701 | cttgggggag ggaggggctg taggtcaaag aaactttata acctagcttt acctcccagc |
| 23761 | tcagccacca gctgccctca aatgttctgg attggaataa gcccaaagat gagtggcagg |
| 23821 | agggaagggc aagccaatac gtctagtttg gttcagtcaa agccttgccc atttcatcag |
| 23881 | aattttaatg gaaaatttcc aataagatta aaatataagg tcaccccaat tttagtatgg |
| 23941 | ctccatttaa aaaaaatcat gcatatcttt gttttgcaat ggggccttac tcttgcccag |
| 24001 | gagaggtgcg gtggtacaat catggcttac ggcagcctca acctcctgag ctcaagcaat |
| 24061 | cctttcacct tggcctgcca aatagctagg attacagaca cccaccacca tgcccagcta |
| 24121 | atttttaaaa aaaatttttt gtaggtccag tgcagtggct catgcctgta aatctcagca |
| 24181 | ctttgggagg ccgaggcaag cggatcacct gaggtcagga gtttgagacc agcctggcca |
| 24241 | caatggtgaa actgccgtct ctactaaaaa cacaaaaatt agcgaggtgt ggtggtgggc |
| 24301 | acctgtaatc ccagctactc gggagactga agcaggagaa tcaattgaac ctgggaggtg |
| 24361 | gaggttgcag tgagccaagt tcgtgccatt gcactccagc ctgggcaaca agagtgaaac |
| 24421 | tccgtctcaa aagtttttgt ggagatgggg tcttgcgata ttgcccaggc tggtctcaag |
| 24481 | ctcctggata tcaagtgatc ctcctgcttt ggcttcccaa agtgttggga ttacaggcat |
| 24541 | gagccaccac aactgaccaa atcatgcatt tctatagaca acgtctgcaa gaagatatta |
| 24601 | atggtgatta tatctggtta tgttggattt gtatttttat ttgtactttc ctgtattttc |
| 24661 | taaattcctg acattctaca tgtgtactcc tttaataatc agaaaaggaa acttaaaata |
| 24721 | gttaaaacca attggtcaga tatgtaaaat aacccaccat ctctccagag agggctgttt |
| 24781 | gctagaactt attttcttca ttgaaatact agagtgcccc aataagtttg aataacacaa |
| 24841 | aaaaaaagat aatgaaagta actaaattat ctaggcccaa aaggaaatgc cacaaaaatt |
| 24901 | ggcaaagaaa caaccatgac gtgctatacc ggatagctcc taggccccct tggagaccct |
| 24961 | gaggtacccg acgagggacc tgtagtaagg ctggcagaca ggttcttcct ctgttagctc |
| 25021 | tgaggtacaa cagttattct catttttatg tctttcacat ggccagaact ttgcaaatca |
| 25081 | gaggcaaagt gaattcagaa ttaaaaattt tcagcaccat ccaagtcagt aaaacagtct |
| 25141 | tagcttataa cctttatttt ttttattatt ttattatttt tttatttttg gatggagtct |
| 25201 | tgctctgttt ccaggctgga gtgcagtggc atcatctcRg ctcactgcaa cctccgcttc |
| 25261 | ctgagtccaa gtgattctcc tgcctcagcc tcccgagtag ctgggacgac aggcatgcgc |
| 25321 | caccacgccc agccaatttt tgtattttta gtagagacgg ggtttcacca tgttggccaa |
| 25381 | gatggtctca atctcttgac ctcctgatcc tcccgcctcg gcctcccaaa gtgctgggat |
| 25441 | tacaggcgtg agccaccgcg cccagccctt tatttttact gtaagctgcc agtaaacagc |
| 25501 | acacccacct gtctgctgac tgtcccatct ggaagttgtg taggtcctta ctgaatctta |
| 25561 | ccttcttccc ttcccctacc cagaccccat cccagctggc acgtggaatc ctttcccttt |
| 25621 | ctcctcatgt caatagacca gcaggcaaag agagcagtta ccatagtgga aggagaaacg |
| 25681 | ggtcctagtc gtggtgagga ggtagggctg ctgcttaaca Raggccgaga gggacatgtc |
| 25741 | tgatgctcag gtgatccact aggacatctc gcaatactcc catgcccagc tgtaactatg |
| 25801 | atggccaagt ccaagaacca tagcctgata agagtctagg acccccacta tacatgccac |
| 25861 | cagtagaagt gcctgatggg gagagggatg tctagaatga gtaggaggga atgatgacaa |
| 25921 | agcacagcca aggaccaact gcagtggcag gtggcagcca cagattgagt taagtaggat |
| 25981 | tcttgctgtg ttccctagtg gaagcatctc ccttgttgag acccactgag gttaaagttg |
| 26041 | gagacatggg gagaaaacaa tgtccccgtg ttcatcacta gagtcatgat gggcaaggtc |
| 26101 | acaactcatt tgacttcttg gctcccagat tcatatactc tgcctcttgg gggcacttca |
| 26161 | ccataataca gccattggtt cagttgtatg ctgcatcctg agggacagca ctccatcccc |
| 26221 | gcagtgccat gtccaaactg ctgccccgcc tcactctcaa gaggctgtcc cactactctg |
| 26281 | tcaggcacca gcttctgaat gcagtggtaa gactctgtcc tgtgatcacg tctatggctg |
| 26341 | attctctttt gccatactga gtcccctgat caaagcagtg ttatacagcc tggcgcgctg |
| 26401 | gctcatgcct gtgatcccag cactctggga ggcagagggg gatggatcac tagaggtcag |
| 26461 | gagttcaaga ccagcctggt caacatggtg aaaccctgtc tcctctacta aaaattcaaa |
| 26521 | aattagctgg gtgtggcatg agcctgtaat cccagctact cggaaggctg aggcaggaga |
| 26581 | atcacttgca cccaggaggt ggaggttgca gtgatccctg atggtgccgt tgcactccag |
| 26641 | cctgagcgac aagtgtgaaa ctctatcgca aaaaaaaaaa gcaacgctta tgattgatca |
| 26701 | cacactgtac tttctaacct gaaggggtct ggtaaaatca acttgtcttc tcaagggaca |
| 26761 | gtaccaaact gggggctcag cactgacctc tgctggcatg tcggacattt aaaagtggta |
| 26821 | gcagctagag cagtcttgaa gacatggagc cctcactgct ggggccgagc actgctgggt |
| 26881 | tcaattctgc taccaaggct tctccatttc cgagcccatg gtgcaggcac tggggtgcct |
| 26941 | aagaggaagt gcactaactg gacttctgct atgcaccttc tttagggggc attaaagcac |
| 27001 | agttcaaaga ctctcacatt ttgagttaac ccccacaggc ctgtgtgccc cttctccagt |
| 27061 | caggtcatgt tgtcatttga tcctagttat tattctgaaa gggttcagga agggaggtgg |
| 27121 | ggacagatct tgRaaagggc aaacattgcc ttgtatgcct catctgaggc ttccgcatag |
| 27181 | tcttagggga ctgggaaccg tcttaagtga atctcacctt cttcccttcc cccagccagg |
| 27241 | ccctagccct actggcatga ggaatcctat ctctttgtcc tcatgccagg agaccagcag |
| 27301 | gcaaagagag cagttaccat actggaagga gaaatgggtc ctggtcatca gaaggaggta |
| 27361 | ctcctcaagg gacagtacct gagggagaca cttccactag ggaacacagc aagaatccta |
| 27421 | cttaactaaa tctacggctg ccgcctgctg ctacagttgg tccttggctg tactttgtag |
| 27481 | tacaaggcga agtaggccac tgctgccagc ccaggaggtc aagggcattt acccagacat |
| 27541 | cctcattccc agtttttggg tcccttatct tccctctcag gatctaatga ctcatctact |
| 27601 | ggcactagga gtccaaaatg accagatggc agactcgcct aggctgagaa ttcttccttc |
| 27661 | tctcaaattc tgccactttt accagcccag tctgcctctg ctgtaggaga tgagaggcta |
| 27721 | aatgttggca gggaggcccc ctgattctga tcctgtgttt taaatcgata attagtcYga |
| 27781 | ccctgttact ttctcccttc agtgcatcaa cgggaggctc ttagcaacag cctcccaact |
| 27841 | ctaccttcct gatagataat attttcctca aagctctcaa atgctagaga cattgcacca |
| 27901 | gctggYgtgt ccccatccac ctgtatctga tcccaggtca ccacaggtga aggtctgagc |
| 27961 | aatcgaactg ctacagcagg ccaggtacca ggactctcaR cacgccactt aacaccaata |
| 28021 | atgaggtcct catggccatt tggcctgcag tgagccaaca ccagaatccc atccattctg |
| 28081 | ggacccctcc tgctactaac ggtcttcagt caggttcccc agaagcagat tatgcaaatg |
| 28141 | tgttattgaa aaacggcttt cagatgaaac ctggaagaca gtgaaggccg aagctagggc |
| 28201 | agggaagaag ccaggcacag acgtgggctt agcagaagtc tagcatcagc ctgatcccct |
| 28261 | gggcagtact ggagcatgga tggcaccaca ggttaccatc ttgagacaga aggactggct |
| 28321 | tctgtaccct gaatcagtaa gccattggtg ggccataagc cacccttagg ggaggacata |
| 28381 | acctcacagg catttcctgg ctggacgacc ctgggcagct gagggcaact gcctgaaggg |
| 28441 | cacaacggtg agccattgtc agctaacctc acagcagcag agacatgggg ccaacagcct |
| 28501 | ataaagaggg tctgggcagg ctgtcaatac tctctactgt aatactgtat atgtggttta |
| 28561 | ttttaaaaac ttttttagaa ggctttattt tattttattt tttgagaagg agtttcgccc |
| 28621 | ttgttgccca ggctggagtg caatggcgcg atcttgactc accacaatct ctgcctcctg |
| 28681 | ggttcaagga attctcctgc ctcagcctcc cgagtagctg ggattacagg cgtgtaccac |
| 28741 | catgcccggc taattttttg tatttttagt ttcatcatgt tggcctggct agtcttcaac |
| 28801 | tcctgacctc aggtgatccg cccacctcgg cctcccaaag tgctgggatt acaggcgtga |
| 28861 | gccaccgcac ccggctgact ttattttttt agagcagttt taggttcaca gaaaaactga |
| 28921 | gaggaaggta caaagatttc ccatatatcc cctgtgctca cacatgcatg gcctccctat |
| 28981 | caccatcccc caccagagag gcacatttgt tacaatggat gaacgtatac actgaatata |
| 29041 | tcatattcac ccaaagtctg tagctcacat catggttcac tttggctgtt gtacattcct |
| 29101 | tggatctaga cacttttata atgacaggta cagtagtccc cctttatcct caggggctac |
| 29161 | ttccaagatc cccagtggat gcccgaaacc gcagagagtg ccaaacttga ctgccatcag |
| 29221 | tgggaatatg tttctattcg ccttccacca ccaccggttt aacgcctttt tcatcttagt |
| 29281 | gctgctgccg taactttggc agtttgagat gcgacagcaa aatgagtaca aatttctttc |
| 29341 | tccttcttca caatgtcatg gacagatgat tccttcttac catagatctt agcaacctcg |
| 29401 | gtgtgtgatt ttttttcttt cttgttaaat caactttcac cttttcactt aaaggaagca |
| 29461 | tttgacggct tctctttggc atatctgaat ttccagcatc acgactgtgc tttggggcca |
| 29521 | ttgtttgttt atttatttat ttattttatt tatttttttg agacagagtc tcgctctgtt |
| 29581 | gcccaggctg gagtgcagtg gcgtgatctc ggctcactgc aagctccgcc tcccaggttg |
| 29641 | acgccattct cctgcctcag cctcccgagt agctgggact acaggcgccc accaccaagc |
| 29701 | gcagctaatt tttttttttt tttgtatttt tagtagagat ggggtttcac tgtgttagcc |
| 29761 | agggtgatct cgatctcctg acctcgtgat cctcccgcct cagcctccca aagtgccggg |
| 29821 | attacaggca tgagtatttt atttatttat ttattttttc agacagagtc tcactctgtc |
| 29881 | ggccaggctg gagtgcagtg gcaccatctc gctcactgca acctccgcct cccaggttca |
| 29941 | agcaattctc tgcctcaaac tccggagtag ctagaattac aggtgcacac caccacgccc |
| 30001 | ggctRatttt tgtattttta gtagagacaa ggtttcacca tcttggccag gctggtctta |
| 30061 | aactcctgac ctcaggtgat ccacccacct cggcctccca aagtgctggg attataggcg |
| 30121 | tgagccaccg tgcttggcct tggggccatt attaagtaaa ataagagtca cttgaacaca |
| 30181 | aacactgtga tcctaacagt cgatttaatc accaagatgg ctataagtga ctaaggctgg |
| 30241 | cggggtgggg agcacagaca gcagggacac cctggacaag gggataattc gtgtaccaag |
| 30301 | caagacacag cggaaggcgc cagatttcat cgcactactc agaatggcat atcatttaaa |
| 30361 | actcatcgat tgtttatttc tgtaattttt ccatttgata tttggacagc agttgactaa |
| 30421 | gagtaactaa aacctggaaa gtgaaacagt ggataagggg gtgctcctgt acttctcaat |
| 30481 | gtgagacatt tgcccttcat gttaattctg ccccattagY tctacacaaa tgaatagcag |
| 30541 | gaaattgatt ttaaaccaca tggtggtaaa atgctttctt ttttctccct catttaacta |
| 30601 | aagaagggct gggccctagg tgatgtcttc cttagcatct aagcagctgg catcacccca |
| 30661 | ctgcttctgt gctccactct cccatgggac cctccctacc tttaatccct cctgtgctgg |
| 30721 | aggccgggtc ttccttgcta gtggtagctc tttccatatt tttaatccat ggtccggatc |
| 30781 | ctgctccact gcctttgctt tagagaaata aacatgaata ttgagtcact ggaaggaatg |
| 30841 | acacacgcat ccctccccca ccagttggag taacgctggc ccacctagtg tatctctggt |
| 30901 | ctaggtctcg aggacctgct gctcctccct cacctgtagt tgaagacctg cctcaggtca |
| 30961 | gtaggtgata cgcagtaagg aaatgtccaa aggacacttc ttgttggatt acacagcaaa |
| 31021 | catctaattg gctgcaaatc tttttttctt tttctttctt tttttttttg agacagagtc |
| 31081 | tcgctctgtt gcccaggctg gagtgcagtg gcgcaatctt ggctcactgc aagctctgcc |
| 31141 | acctgggttg acgccattct cctgcctcag cctcccaagt agctgggact acatgcgtgt |
| 31201 | gccaccacac ccagctaatt tttgtatttt tagtagagac gaggtttcac catgttggcc |
| 31261 | aggatggtct tgatctcttg accttgtgat ccgcccgcct tggcctccca aagtgctggg |
| 31321 | attacatggc tacaaatctt aaagggggaa gagatgagga ggaataatcc cctttgtctt |
| 31381 | ctcaaaaatg ttttcactgg ctcactacag ctccctcctt cctctttact gacccaaaat |
| 31441 | gccaactatt atagtaactc ttttgggtta gacgaatcca gtgaataaac acctactaag |
| 31501 | cattggaggt ccaagaggaa taagatatgc ctgagctaaa cctcatcacc cccggccttg |
| 31561 | cttgcagagt ggtttgctgg cctcatctga ttattcaatg agtgctttca tttgtttatt |
| 31621 | cactaaatat ttactgagca cctacaaaag tgcctggccc tggtagatga ggcctgaggg |
| 31681 | aagctaaaac taataagaca aactccatgc cctagaggaa ttcatggtct catagggaga |
| 31741 | aaatgtaaac acatcataaa attatagctt attaaatgct gcaatagagt actccgtaag |
| 31801 | agtgtggggg cagagaggag ggggagaaac agctgctttc tagagccctc accttttcca |
| 31861 | cttctctcat ctttctgggt tagggtctag cgggggttcc atgaggatca ggctaaatga |
| 31921 | gcttagaaaa actaagcaga ctactgtatc agaactggat cacagtagac aggcgttttc |
| 31981 | aacaaacata tattgagtat ccctgagtgc tttggacagg aagaggaatt atagacgaag |
| 32041 | attgtaagtc gcagtaatag atgaggcaag gagcaccagt gaggacttaa ccctgaaaga |
| 32101 | agtgtgagca catcttcctc ccagacaagg gggaataaag aaaggaagat gatcaggaga |
| 32161 | gttctaagtg gaactcagca gccagagggg aagcYggagg aggtaacatc agagggtgcg |
| 32221 | tttgcccact cagtaaagta ggaggcaggg cagccttgtg aaaataggaa tggaatagaa |
| 32281 | agctcaagaa aacagccaaa agcaagggca attaggggag gtttcgaact ggcagatcta |
| 32341 | cctcaactgg caatacagca agcatgcacc agaaaagata tcctagctag cagtggggtt |
| 32401 | gggaactgat ttaatatcct aggctagcag tggggttgSg aactgattta ttatttaatt |
| 32461 | ttttatatct attcatgtat ttatgcactt atttattttt gagataggct ctcgctctgt |
| 32521 | cacagaggct agagtgcagt gatgtgatct aggctcactg cagctttgac ctcctgggct |
| 32581 | caagtgatcc tcccacctca gcctcctgag tagctgggac tacaagcacg ccaccaccta |
| 32641 | ggctaatttt tgtatttttt tttgttagag acagggtttc accatgttgc ccaggctggt |
| 32701 | ctcaaactcc tgggctcaag cgatccatct gccttggcct cccaaagtgc tgagattaca |
| 32761 | ggcgtgagcc actgcgccca gccaatattt tttaaaaatt ggaaatccct tgtaataagc |
| 32821 | caagtgttgg gggaagaaaa gcaataaaag caatgacatg gactcaatat gaaacatcca |
| 32881 | aagcatttga catgccttta aaataaaaaa atcagtactc acctcgtgct catttcaaac |
| 32941 | tgtggatttc cttggtctaa aattttaaaa aacaaaaaac agcactctca aatttaagct |
| 33001 | aatttgaatt gattttacat agtgattttt aaatacacat atatgcataa agagaatact |
| 33061 | ataaaaatat ataaaggtgt gtaggtcttg atcatatttt cccaggaatt cagaaaacgc |
| 33121 | tgctacagtc cctgggctca cgtggtcctg gcatcctccc caacgtctct accccatcct |
| 33181 | gctgagtttc tggcatgcat tggttctggg tctgtccaag tttctactgt tagctccatt |
| 33241 | aatactgaat taaggaataa acagtgctca aatgctcatt ttttccatgt gagcccaaat |
| 33301 | tatgtgactt gatgagggaa aaaatcatga gtccctggga gagcgataag aatcacattc |
| 33361 | tttactaaag tgttgtccca ggtatgagaa taacaccaga tctcaacctc cagaggcccc |
| 33421 | ccactgcctc ccacagcata aaagccaaat tcctcggccc gatatttgag gccatctcaa |
| 33481 | tctttttcct ttcaccctat acctgactct cccaggctag gctcagatcg tcactgaatg |
| 33541 | tgctcacttc gaggcacctc tgtgattttc aaggttcctg ggcccacctt ttactaccga |
| 33601 | tgtgactgcc acatgttgcc cagttgctag gatgggaccg tggccttgat ttctgccggg |
| 33661 | actgaggctt tgccttgttt cccacccacc ctgctgcctg cccctgcact cttctggctg |
| 33721 | gggcctgatc tttccccgca gtctcccttc acctctagat cacaggcagt catgccacag |
| 33781 | ctgaggagct tgtcccaaac ctcagtgcct gcctccctcc tcggcttctt gtgctgctgt |
| 33841 | gtctcaccca tcagtgaYgc tcttctcttc cctgcccaag ccctagctga ctccataaca |
| 33901 | cctggataca atgtcctctt ctgtctggtt acctccgaga ggccctctct ctacatccct |
| 33961 | cattggctcc cagtgtcatt cctctcaccc atggccctaa ggtcactgta ttctttggcc |
| 34021 | agtgtacagg gttattatgc ttaacaatcc acaaaggttg aaaggtgttg taggatggtg |
| 34081 | taaaaatgaa tctgggtggt aatgtttata tgtcagagct ttgtaaagtg ctcggcaggc |
| 34141 | gtaaggtact gacagtcctg atattcctga tcttggaacc tgggacacca tcttctcaac |
| 34201 | attgcccgga taccctcaag ggtatccaga cagcctgagt ttgcattctg ttcctggttc |
| 34261 | agcccagggc cctggttccc gctcactcac catccactgt gggccctctc taaattctta |
| 34321 | aagcccttgc catttgcatc actcacagag acatttcatc agagcctact tggtgcacca |
| 34381 | ggctcaggag actcagttct gctgcggata agttatgtaa aattgaccct ctgcttgcac |
| 34441 | atctgtaaaa ggaaggggct ggcacaacac ctctgggcct ttcagttcag tagtgttttc |
| 34501 | tttttatcta aaccacgtgc tgggtcctgg ttttgcttct tatctagatt tttgcattcc |
| 34561 | tgtcacaacc tataaagcac agttcaggcc ttaaggaggg cttgagaaat ctctttccga |
| 34621 | attgtcaact gaataagtgt catatcttca taacaaactt gtcttttttt gcagggccag |
| 34681 | gaaggcagca ggggagtcag ttaaaatata aattttagat taagcttaat attgttaaga |
| 34741 | agtcaattct caccaaattg ttctagagat tttacataat cctaatcaaa atctcaataa |
| 34801 | ggttttttga gaagttggca agcagattct aaaatatata tagaagtata aaggacacag |
| 34861 | aataatcaaa acaactttaa aaaggaagaa caaaattata ggactctaac tacctcattt |
| 34921 | taagacttat tagaaagcag cagtaaccaa gatagtgagg gactgatgtc aaggtagaca |
| 34981 | aatagatcaa tggaaaagaa tcaggcatcc agaaatagat gcacttacat aaatcatcaa |
| 35041 | ttggagaaag aatagtgttt ttaacaactg gcaatggaaa aactagaaca tcagtatgca |
| 35101 | aaggactgaa ctttgatcca tacctcacac cacatacaaa tcaaaaacag aaacaaacaa |
| 35161 | acaaacaaac aaatactcaa aatggatcag agacctaaat gtaaaactat aaaacttctg |
| 35221 | gaagaaaaca cagggagaaa atctttatgg cttttagata acgatttctt aggcaggata |
| 35281 | ccgaaaacat gatacataca gtttttaaaa ttaaatatta taagaattaa agtcttttgc |
| 35341 | tcttcaaaag tcactcttaa gagaaaaaga tgccacacac tagaagaaaa tatttacaaa |
| 35401 | gcattaaaag gacatatatc taggatatac ataaaaactc tcaaaagtca ataataagaa |
| 35461 | aacaatgagc aaaagatttg aacagacact tcaccaaaga agagataaag atggcaaaga |
| 35521 | agcacataaa gagatgctca accattagtt actagggaaa agcaaattaa aacctaatga |
| 35581 | cataccacta tgcccctatt agaaatctga aaatttaaaa gactgacaat accaagtatt |
| 35641 | ggtgaggaca tggcacaagt ggaactctca tacattacta tgggaatgga agatactata |
| 35701 | atcactttgg aaactattta gaaatttcct aaaaaattaa acatacacct accatatggc |
| 35761 | ctaaccatta cactcttagg tatttaccca agagaaatga aaacacatgt ccacacaaag |
| 35821 | acttgtactt gcatgttcat agcagcatta ttcaaaatag ccccaaagca gaaacaaatc |
| 35881 | ggatgttcat taacaagtaa atggataaag aaaacggggt ctagccaaac aatgaaatac |
| 35941 | tactcagcaa caaccaaaat atgtactatt gtttacaaaa tccaaataga tgaatctcag |
| 36001 | aataattatg cagaggagag aagccagacc aaaaaaaaaa gtacatagtg tattatctct |
| 36061 | tatgaaattc tagaaaatgc aaactaacct atagtgacag aaaggagatt ggtggtcacc |
| 36121 | tggggtgggt gagggagggg caggagaaag ggaatgcaaa gcagtgtgaa caaacctttg |
| 36181 | gcggtgatag gcatgttcat tatccttact gtggtgatgc cttacagatg tatacagatg |
| 36241 | tcaaaactta tcagattgta cactttaaat atgtgtggtt tatcgtgtca ttatacctca |
| 36301 | gtaaaggagt tttaaaaatt gtagtaaaag gtctctacct agaaacctta ataagctaaa |
| 36361 | atgtatgtcc ccaggactaa ccctagctat tctatagttc tggggtggaa cctagaaatc |
| 36421 | tgcatttgta gcaagccctt caaataattt gaatgcaggt ggtccttaaa ccaccctttg |
| 36481 | agaaacactg acctagtgag taaggatttc taaacaagct tgtgactaga atgttgtttc |
| 36541 | tgcagggaac aagtgacttt ttctactaga gttgccatat cattctgtgt taagcctcta |
| 36601 | ggacactcct actcaattac catgacatat atttaacata atattaatag ttgtaaaaca |
| 36661 | aaagtaaacc atagtttttt tcccatctta tctgtataca aagtaaaatc aatgacataa |
| 36721 | tattgttact agttcctggc agttacctac agtttaaatc aagaacttat gttgcttggt |
| 36781 | ttttgtgaca gaaaactgat gtggtggctt tccagttaca ctgcttggtt taccttttcc |
| 36841 | cttatcttat gatgcacacc cagctttgca cgttcctgag cagctgatga tcatagaaga |
| 36901 | cctagaaaaa gtgtgagcca gttgcattcc tgctatctct aaagaaaaaa gttatctttt |
| 36961 | catttttatg tgatttttct tacccaaaat gcaaaccact gccagtataa atgatggaag |
| 37021 | atagactttc cacatgaaac tggcatttct tcaccctagt aacattacct gaggggaagg |
| 37081 | tcagaactca attgataaag ctcgctgtgg attttcccac caaatatccc tgcaaacaaa |
| 37141 | gtgaaaggat aaagcctgct taaagatgat ttgtaattgt tgaaaggagt acagtgtgtg |
| 37201 | acatcagtga aaatggtgga gaaaaaaccc gtccccaaat tctcttcttt gtgaaagcat |
| 37261 | ggaaaaaact ggccaaaaat ggttagaaaa tttattttca gaatatcgaa attttttttt |
| 37321 | cctttttttt ttttttatta tactttaagt tctagggtac gtgtgcacaa catgcaggtt |
| 37381 | tgttacatat gtatacatgt gccatgttgg tgtgctgcac ccattaactc atcatttgca |
| 37441 | ttatgtatat cccctaatgc tatctatccc ttccccctcc ccccacccca ccacaggccc |
| 37501 | tggtgtgtga tgttccctac cctgtgacca agtgttctca ttgttcagtt cccacctatg |
| 37561 | agtgagaaca cacggtgttt ggttttctgt ccttgcgata gtttgctcag aatgatggtt |
| 37621 | tccagcttca ccatgtccct acaaaggaca tgaactcatt gttttttatg gctgcatagt |
| 37681 | attccatggt gtatatgtgc cacattttct taatccagtc tatcattgat ggacatttgg |
| 37741 | gttggttcca agtctttgct attgtgaata gtgctgcaat aaacatacgt gtgcatgtat |
| 37801 | ctttatagca gcatgattta taatcctttg ggtatatacc cagtaatggg atggctgggt |
| 37861 | caaacgatat ttgtagttct agatctttga ggaatcgcca cactgtcttc cacaatggtt |
| 37921 | gaactaattt acagtcccac caacagtgta aaagtgttcc tatttctcca catcctctcc |
| 37981 | agcacctgtt gtttcctgac tttttaaaga ttgcctttct aactggtgtg agatgttatc |
| 38041 | tcattgtggt tttgatttgc atttctctga tggccagtga tgatgagcat tttttcatgt |
| 38101 | gtctgttggc tgcataaatg tcttcttttg agaagtgacc gttcatatct tttgcccact |
| 38161 | ttttgatggg gttgattttt ttcttgtaaa tttgtttaag ttctttgtag attctggata |
| 38221 | ttagcccttt gtcagatggg tagattgtaa acattttctc ccattctgta agttgcctgt |
| 38281 | tcactctgat ggtagtttct tttgctgcgc agaaactctt tagtttaatt agatcccact |
| 38341 | tgtcaatttt ggcttttgtt gccattgctt ttggtgtttt agtcatgaag tccttgccca |
| 38401 | tgcctatggc ctgaatggta ttgcctaggt tttcttctag ggtttttatg gttttaggtc |
| 38461 | taacatttaa gtctttaatc catcttgaat tcatttttgt ataaggtgta aggaagggat |
| 38521 | ccagtttcag ctttctacat atggctagcc agttttccca gcactattta ttaaataggg |
| 38581 | aatcctttcc ctatttcttg tttttgtcag gtttgtcaaa gatcagatgg ttgtagatgt |
| 38641 | atgatattat ttctgagggc tctgttctgt tccattggtc tatatctctg tttttggtac |
| 38701 | cagtaccatg ctgttttgat tactgtagcc ttgtagtata gtttgaagtc aggtagcgtg |
| 38761 | atgcctccag ctttgttctt ttggcttagg attatcttga caatgcaagc tcttttttgg |
| 38821 | ttccatatga actttaaagt agtttttttc caattctgtg aagaaagtca ttggtagctt |
| 38881 | gatggggatg gcattgaatc tataaattac cttgggcagt atggccattt tcacgatatt |
| 38941 | gattcttcct atccatgagc atggaatgtt cttccattgg tttgtgtcct cttttatttt |
| 39001 | gttgagcagt ggtttgtagt tctccttgaa gaggtccttc acatcccttg taagttggat |
| 39061 | tcctaggtat tttattctct ttgaagcaat tgtgaatggg agttcactca tgatttggct |
| 39121 | ctctgtttgt ctgttattgg tgtataggaa tgcttgtaat ttttgcacat tgattttgta |
| 39181 | tactgagact ttgctgaagt tgcttatcag cttaaggaga ttttgggctg agacgatggg |
| 39241 | gttttctaaa tatacaatca tgtcatctgc aatttgacaa tttgactttc tcttttccta |
| 39301 | attcaatacc ctttatttct ttctcctgcc tgattgccct ggccagaact tccaacacta |
| 39361 | tgttgagtag gagtggtgag agagggcatc cctgtcttct gccaggtttc aaagggaata |
| 39421 | cttccagttt ttgcccattc agtatgatat tggctgtggg tttgtcataa atagttctca |
| 39481 | ttattttgag atacgtccca tcaataccta gtttattgag agtttttagc atgaagggct |
| 39541 | gttgaatttt gttgaagacc tttactgcat ctattgagat aatcatgtgg tttttgtctt |
| 39601 | cggagaacac tggaaattaa atgatggctt gcagcaatct ggagagcatt tattcaagga |
| 39661 | aaatggctgt gtctcagtat gactaatgag ctttttaact tgccctattt ctatcctccc |
| 39721 | cttccttggt ggtagcctta gaaatgaaca gcctgcaatg atagtgaaaa tcagcagtct |
| 39781 | ggcagccatg ggaggggcag aacaggaatg ggggaactat ggagcctcat tcttagagaa |
| 39841 | ttatcattat ttgatctgtc cacgggtttc taggaatacc tgacctgcaa gtctgtcttt |
| 39901 | attaggcctg actcagaact tgcccaatgt gaaaagtctt ttccccaaag gcctttgtag |
| 39961 | aaaatgatta caggcaattg tttaacttct tggttgctcg aggtattggc taacagtggg |
| 40021 | gcaaacatgg gctaatcaga aggtttaaaa tgaaatgctc aggaataaga tgctcataga |
| 40081 | gggttgtaaa ggctccaaaa tatttatgag actctagaag accatgcaca catttcctgt |
| 40141 | gaacatgttc aggacagatc tgcgaaggcc ccacgatctt acctctggct gatcttgatg |
| 40201 | atctgcacaa acagaaagtg aaagctaggc tagaactgtc aagtgccagg ctgagtgtga |
| 40261 | aggtgtgccc taatgtgcac acagagcccc ttggcaaaga ctagaagact tactgctttc |
| 40321 | aggtgtttaa agaaatctct gtcatgtcat tagtatttag atcactaagc taactgaaca |
| 40381 | gaggcttcaa tggctgcaca taaatacaga cttcacagaa ttagtttaga aaagtcacta |
| 40441 | taacaaacaa caataaacag caacaccaac aaacagtaga ggtgggaagg tccaatttcc |
| 40501 | agagttgcta cattatgtta tttaaaatgt gcaattttaa cagaaaataa tgagacatgt |
| 40561 | aaagaaataa gaaaatgcag tccataccca gggaaaaaga aaaaccagtc aatacaaact |
| 40621 | gttcctaggc caggtgcagt ggctcatgcc tgtaatccca gcactttggg aggccaaggc |
| 40681 | aggcagatca ccagaggtca ggagtttgag accagcctga ccaacatgtt gaaaccccat |
| 40741 | ctctactaaa agaaaaatac aaatttagcc aggcatggtg gcgtgcacct gtaatcccag |
| 40801 | ctactcggga ggctgaggca gaagaattgc ttgaacccag gaggcggagg ttgcagtgag |
| 40861 | ctgagatcat gccattgcac tccagcctgg gctacagagc aagactctgt ctcaaaaaag |
| 40921 | aaaaaaaaag aaaaaagaaa aaactgttcc tgaggaagcc aagatactga ctttactaga |
| 40981 | ccaatacttt aactattttt acatgttcaa aaagttaaag gaaatcatat ataaagacta |
| 41041 | aaggaaagca caagaacaaa tcctcattaa atagaaaata taaagatttg ttttaaagga |
| 41101 | cgaaacagaa attctagatt tcaaaagtat aataattgaa tgagaaattc actagagtgg |
| 41161 | ctggctcaat agcagatctg agcaggcaga agaaagaatt agagaactca gaaataggtc |
| 41221 | aattgaatct atccagtctg aggaagagaa agaaagagga atgaaggaaa aatgaataga |
| 41281 | gcctcagaga ctgtgagata ccttcaagca cgctaatgac gcataatggc agtctcagaa |
| 41341 | ggagagaggg ataaatgggc taaaataata tttaaagaag caatggctga acatttccca |
| 41401 | aatctgatga aaacattaat ctataccttc aagaaagtct ataaactcca agaaatataa |
| 41461 | attcaaagag atcaagatcc acacctagag ataacataat caaactgtca acaaagacaa |
| 41521 | tgaaattatc ttgaaagcag caagcacata gaaggactct tcaataagat taacagctga |
| 41581 | tttctatcag aaatcacaga gttgagaagg caatgggatg acatattcaa agtgcttaaa |
| 41641 | gaaaaagagg gtcaacaagg aattctatac ctaaagccaa tttatcttca acaaagatgc |
| 41701 | caagaccatt caaagYgggg aaagaatagt cttttcaatc aatggttcta ggacaatgga |
| 41761 | tatccacatg taaaaaaagg aacttgggcc cctaattcac atcatataca aaaattaact |
| 41821 | cggaatgagt caaaggctta actgtaagag ttaaaactat aaactctttg caaagaacac |
| 41881 | tatcaagaga gtaaaaagac aatgcacagt atgggaaaaa atacttgcaa ataatatatc |
| 41941 | tgataaaagt ccagtatcca gaatatataa ataactctta caattcaacc ataaaatgac |
| 42001 | aagccaatta aaaaacatac aaatgagtta actgacattt ctacaaagaa gatataccaa |
| 42061 | tggccaatat gcatgttaaa agatgctcaa catcactagc catgagggaa atgtaaatca |
| 42121 | aaatcacaat gagatactag tacatgccca ctaggatggc aatgataata ataataataa |
| 42181 | taataatgtt attataatga acaatacaag tgttagtaag gaaatggaga aaattgaacc |
| 42241 | caactattat attgctggtg tgaatgtgaa atggtggtac tgttttgaaa gatgatttgg |
| 42301 | aagttcctca gaaagttaac aaaagttacc atatggtctg gcaattctac atatatacac |
| 42361 | caaagaaaac tggaaataaa gattcataca aaaatctgta caaaaatatc tacagcagct |
| 42421 | ttacttacag tatccaaaaa ggggaaaaaM cccaaatctt catcaactaa tgaatggata |
| 42481 | aacaaagatg gtatatccat acaatggact gttactcagc cataaaaagg tatgaagtac |
| 42541 | tgatagatac tacaacataa atgaaactta aaaaaatacg caaagtgaaa gaagctagac |
| 42601 | agaaagggcc atatatgtga tgtgtagaga aaacatccaa tagagacagg aaatacatta |
| 42661 | gtgattgcca ggggctgggt aaaggtgaga gatggagaat gattgctaat tggtacagaa |
| 42721 | tttttttgtt gggagagtta tgaaaatatt ttggaagtat aattagaatt aggctgttaa |
| 42781 | atactttaaa acaaaaaaca gctgggcgca gtggctcaca cctgtaatcc cagcactttg |
| 42841 | ggaggccgag gcaggcagat cataaggtca ggagatcaag accatcctgc ccaacatggt |
| 42901 | gacaccctgt ctctactaca aacacacaaa ttagccaggc gtgttggcgc gcctgtagtc |
| 42961 | ccagctattc gggaggctga ggcaggagaa tcacttgaac ctgggaggca gaggttgcag |
| 43021 | tgagcccaga tggtgccact gcactccagc ctgggcgaca agagcaaaaa ctccatctca |
| 43081 | aaacaaaaca aaacaaaaaa caaaagtaag aagtaaaaaa atagaattag actggggatg |
| 43141 | gttattgagc cttgtgaata atctaaaacc cactgaattg tatattttag acagtgaatt |
| 43201 | tgatggcatg ttaattatat cttgatgttt taaaaaaagt agtacagtgt ctgaaaggta |
| 43261 | gcattggcca tttgaatctc tgttaacatt ttcttgggtg gccatcagat ttttgattaa |
| 43321 | aggtgacctt ggcaattaag tggtgggggg aacacaacca aaagggacaa accaacacaa |
| 43381 | ccacacacaa tcacacaacc cctccacctc caccacagtg gaaatcaagc ataggtgctt |
| 43441 | ccagggactg tgaaaatgtg gccacagttg ttttcccagc aaaccatttt ctttcacaat |
| 43501 | atccccaatt tgcacatccc ccactgagct gaaatcagta taactcgaca gacRaaagtc |
| 43561 | agcaggggta acctaccttt gatattattt tgttgttcca tcaataattt acttatttct |
| 43621 | gaaaaccaac agtctctgat ttcttttgaa gctgcctgca atcacacata agaaaacact |
| 43681 | ataagactac catgagtatt aattgcagag tttttattct cccagtgacc gttagactaa |
| 43741 | tgagatagaa aacacttgtc aagtcatggg aacacaataa ggaaagagta gaggagaccc |
| 43801 | atggggaagg catgagcgtt acctgcagga tgtatttctc aagtccattt cgactggcaa |
| 43861 | tctcaaactt tctatggctc ccccttccaa gctggcgaat tgaaagtgtc atcagctatt |
| 43921 | ataaagaggg aagagaaatg ttctatacca tcgttttctg attttaaaat agtccctgtc |
| 43981 | aatcactcta atcctttcta tccttgacag ctctcttctt tcatcttcct ttcaagtttt |
| 44041 | tgctttcctc tcttaatgac tccaagacag cataaaacac aatctggtac agtgtcttct |
| 44101 | gtctcatctg tttcagaagg taacacagac tgttaacttt ctactgtatc tttatgcagg |
| 44161 | aggcgctgca gccctcaacc tttcagtgaa gagatcattt ctccttcaac Rtgtatttta |
| 44221 | caactttcta cgtaatagta actgaataag gatctattca gaaacataaa taacaaggcc |
| 44281 | aggtatggtg gctcatgcct ctgtaatcag tcctgtaatt ccagcacttt gggaggccta |
| 44341 | ggtgggcgga tcacctgagg tcaggagttc gagaccagcc tgaccaacat ggtgaaaccc |
| 44401 | tgtctctact aaaaatacaa aattagctgg gcatgctggt gcgtgcctgt aatcccagct |
| 44461 | acctgggagg ctgaggcaga attgcttgaa cccgggaggt ggaggttgca gttagccaag |
| 44521 | atcatgccat tgcactccag cctgggcaac aagagcaaaa ctctgtcgaa agaaaagaaa |
| 44581 | gaaagaaaga caagaaagaa agacagaaag aaagaaagga aggaaggaag gaaggaagga |
| 44641 | aggaaggaag gaaggaagga aggaaggaag gaagggaggg agggagggag gaaggaaggg |
| 44701 | agggaggaag gaaggaagga aggggagaaa gaaaacaaag gctactatga acaaattaac |
| 44761 | agctttaatc acaaSagcat aaatatatat gggtgtggct cctgtatgtg ggtgtgtgtg |
| 44821 | tttcatagcc tgagagctaa ctggctatga aacagtttct aaacaggtga gaaaacaatg |
| 44881 | cttggtgtgg gcctgggtgc cttagacccc aaggtcagga caatgcctgc acttagacac |
| 44941 | aaagatacac cgttgagaag gRcaagtcta agatgaaatc cagcccatgt tctcatcact |
| 45001 | aagtctttca actctctttt acctctttgc ctggaagaaa gagggtactt ttactaatta |
| 45061 | gtttgtccag actggctgcc atatagtctt ttttctctag tacaaaggca ctcctatgcc |
| 45121 | agccctggcc cataagctct gaccctggag tcagacagac ccggctatgg attctagccc |
| 45181 | tgtggtgtgg tatttggcaa gttaacttca cctctctaca ctgcagcttc ctcatctgta |
| 45241 | aaataaggaa aacgatctct ctcttttctg catagggcga ctcccagatg caataggact |
| 45301 | ccctttacct tcatggYctt cttgaagctg taatgaggag ataatccctg gtccccaggc |
| 45361 | tccattcgta tcttacagaa cactattccc ctttcaaata ggtagatttg cctctggctg |
| 45421 | ggtttaaatc gaatcaaatc cttcatttta taacgatcct tgtgaattgt ccagacgctg |
| 45481 | aaagggccgt gcagcaacag cttgcctagt tttccaatat cgtccttttg gaaacacaca |
| 45541 | tacaggaaaa gaagctgtaa aattacttga cagagaaaga agcctttgtg gcctgttttc |
| 45601 | aaattttata aagcatgctt ctttgaggtt tacaattcta aaagtgctat tcaggctggt |
| 45661 | ctgtcactaa atcaaaacga caattttttg agcttcactg agcgtggtgc cRtttagtta |
| 45721 | tataaggaaa tcaattcagc tatgaaaggt aatgctatac ataaagaatg ttagaaatgc |
| 45781 | ttgagctaga gatacgggga ctgtgatatt tttgactcaa ttttaactag cagaattcaa |
| 45841 | tttcctctgt gctatgtttg atacatgaca gtctccttca aagggacaag aaattaacat |
| 45901 | ttcaattatg tccagtagag atagaatttt ggctgcattg Yggacccatg agcatctgtg |
| 45961 | tgttccatct gcatcctaac ttctgcttca ggtatatgag gacctgagaa aatgaagctt |
| 46021 | gttctgaaaa gagaaagttt actgtgatct aatcttcaca acattgtaat ggaaatagta |
| 46081 | actgcattca actctgagtt tttcagagtg agtcaacagt gccaagataa taccaaatga |
| 46141 | cagattttta agaagtctgt ttgactctag gcataaactg tgatgcccct gtctccacca |
| 46201 | gcaaggctgc ctttgtcata tgtctggttg aaagtccaaa tgagatgacc ctgccttctc |
| 46261 | tgagctctct tcaaaagaca gtggaccaag atgagctggg agcgaggcat tttatctggt |
| 46321 | atttttcaaa acttcgattg cttaaagcag ataagaattc atttccctga ataaaaacac |
| 46381 | caaaaggaac aaattatttg aaggggacca agacaaagaa atctccctaa gtgacctcta |
| 46441 | gctgagatca atccctatga tttctgagtt aatactggaa atatgtactt cattcataat |
| 46501 | tgattgggca tttttataaK ttattgttta tatgttgggc aaactctagg tatctttttt |
| 46561 | aaaaaggaat tgactttact tctgaaacat taaaagaaaa acttcaagag caataaactc |
| 46621 | tagtcatttc tatggtgtct atggcgttat ttcctatgtt aattttctat tttcttcttt |
| 46681 | ggaaaattta catatgtaga ctgtggtgaa taaaaccctc aaactcccag ccacgaggcc |
| 46741 | ttctttccta aaagccaatg ctcctgttct gtgttaacag ttcaccacat tccaaaacag |
| 46801 | aagggtggat aggtccatga ggaccctgga gaattttttc actctatctg catccatcaa |
| 46861 | ggacccactc aaatgtaatc actacagacc ttagtgactg ctttttcctt taaatccatt |
| 46921 | caattcataa tcaatatcga cataaatagt gcaggaaata tgttttagta aaggcaactg |
| 46981 | gtagaagtag atacttaata tttgccacat aaacaaatgt tatcagcaca ggttgagcat |
| 47041 | ccataattta aaaaatctga aatcctccaa aatctggaac tttctgagaa ccagtatgac |
| 47101 | actggaggtg aaaaattcca tacctgacca catgtgatgg gtggcagtca aaacacagtc |
| 47161 | aaaattttgc ttcaYgcaca aaattatttt ttatttttta tttttttgag acggagtctt |
| 47221 | actctgttgc caaggctgca gtagagtggc acgaccttgg ctcaccgcaa cctctgtctc |
| 47281 | ctgagttcaa gggactctcc tgcctcatcc tcccgagtag cttggattac aggcgtgcat |
| 47341 | taccatgccc ggctaatttt tgtattttta gtagagccag ggtttcagca tgttggccag |
| 47401 | gctggtctcg aactcctgac ctctggtgat ctgcccacct cagcctccca aagtgctggg |
| 47461 | attacaggca taagccactg ctcccggcct tcatgcacta aattatttaa aatatgtaaa |
| 47521 | attaccttca gtctatgtgt ataaggtgtc atgaaacata aatgaatttc gtgtttacac |
| 47581 | ttgggttcca tctgcaacat aactcagtac gtatgcaaat actccaatcc tcccctaaaa |
| 47641 | aaaaaaaaac tccaaaacac ttttggcctc aagcattttg gctaagggat attcaaactt |
| 47701 | gtagtttcta tcttacaatg tattcactct ctacctcatt ttaaacattc gacgcatctt |
| 47761 | acagaaatat atacagtata gacagataga aaataaccga gggaattaag gtgaagaaaa |
| 47821 | aataaagaag ttgggagctt ggcgcctgcc tgtagtcgca actacttggg aggctgaggc |
| 47881 | aggaggatca cttgagtcca ggagtttgag gccagcctgg gcaacatagg gagatcacca |
| 47941 | tccctaaata taaagacaat aaatcagatg aactgttcca gaaggtgtag aagataaaag |
| 48001 | aaaagatgaa gaggttatat gtaaaatgca tataataaag ccatKtaaac ttgatgtaaa |
| 48061 | cttggctctg actttctaat agcaatacag agggaaacat ggtgagtggt acgatttatt |
| 48121 | atgtctgaag acatacacaa accggttgtt caggaaaagc acagctattc ctactaagac |
| 48181 | ataagaaaca tttctcccag tgatatgttg tcactgagca acatcctcaa ccacgcccta |
| 48241 | tgatagaaag aggaacatgt tttaggtaaa gctgcttgta agaacacatt tggtcagtgt |
| 48301 | ggaggctcag ctgtcaaagt aattctagaa gaggctagga tgatagggga agggggaata |
| 48361 | agtcgaggtg tacactctct aatgacttgt gaagatgggg tgacgtgagt taaggcatcc |
| 48421 | acaaggctat tgtagaggaa acagccccaa ccagaaatgc tgaagaggca gcctcaggca |
| 48481 | gcaaggtttg tgctttatta cctctgaact ttcctcatca ccccatccac agccatgctg |
| 48541 | ctgcaccttc ataggcaagt aatcagagga tgtgaatcca gagaggtacc agcagctgac |
| 48601 | catagggccc ccagcataaa aacaatgtgc cagggKtggg ggttggcaat gtgatctctc |
| 48661 | cctgaactga agaaaacagg aggaagtgRt gaccagcagg agaYggggct gtgagatgct |
| 48721 | ttaggggaaa gcaggcagtg gaataaacga gaaggaaaca gaactatgaa gaaggtgagt |
| 48781 | cattagaaat tatgaggaag aggaagaaac tgacggaagg gaaaaaaaca gtcagtaggt |
| 48841 | ggtaagagSR ggggcgagca ggcatgtggg tgcacttgac atatacaggc gggtcacaca |
| 48901 | ggtaagaacc ccggctccct ccccaggccc aggccccaga ggtgctgtgg atcctgggtt |
| 48961 | tgctcagttc ccatctccgc gaacttggct ttgccatcgt gacttcttga gaagctgcag |
| 49021 | gtcagctttc cagtaagccc attattactg gaagtgactc ggggggagtc gctgggtctt |
| 49081 | tcaagtctgg cagaaacata tgggcacata tcctctgggt catgttgcac aaatgttctc |
| 49141 | aggaccacac atatgacggt attgcccatc tccacgccca aggtccgatc tccagcgggt |
| 49201 | gagcacatgc cctacacggt gctgaccaag ggcaggcaca cacactgttc actgtgctgt |
| 49261 | gtggagctgc tccgcctcca acaggaagct gcagggacat tccctgagca ggagcaagaa |
| 49321 | tgaccttttc cgtcacgtgt accttttctt tccaactaga ctgtagttga tctcaaggtc |
| 49381 | ggagaccacg ttgcacaacc tctttcagat acgtgttcac cagtatttat ttatttattg |
| 49441 | gcattcccta gtatttattt atttattggc atttagtggt gcttgggagt aggggacagg |
| 49501 | aaacaaaata ctcatcactg tgagggaata ttgatttatc caaggagttc attcagcgac |
| 49561 | gcccctgtac tgggttgaat agtgttcccc caaaaactca tgtctatgag accctcaaaa |
| 49621 | tatgatctta ttcggaaaga gggtctttgc agatacaatt ggttaagatg gggttgccct |
| 49681 | gggttaggat gggtccaaat ccaatgactg gtatccttat aaaaaaagaa aacaaagaaa |
| 49741 | tggagacgca cgctgaggga acagccatgc aaaggcagag gcagaaattg gagtggcaca |
| 49801 | gctgtcagcc aaggaactcc aagaattgcc ggcagtcacc agaagctcag acgaggtaag |
| 49861 | gaaggattct tcctgagagc cctcagaggg aacgtgggcc tgctctcacc ttgcctttgg |
| 49921 | acgtctagcc Ktcaaaactg tgagggaata aatttctgtt gttttaagtc acgtactttg |
| 49981 | tggttccttg cgacatcagc cctaggaaac gaatatactt ccaactgttt tccaccagta |
| 50041 | acaaagaagt gtggccatgt cacacggtgt gtggaagaac ctgatggttg tgaactcaga |
| 50101 | gatggaacat agcggctacc tcaaatgagc gagcgataag ttagatggcc tcctctgtac |
| 50161 | aagaagtgat agcaaaatca attagtaaat gctaccactt tcacacctgg gcattctgca |
| 50221 | ggtaagagcc ccgatacaag gatttcaaat gtgactacat tggccaaatg aagagtctgg |
| 50281 | ctgagagagt gaatctaggg aagctggcct cagcaagagc ttcttttggg tgcccgtgtt |
| 50341 | tccattccag gtagtaagag ttaatatgac tgtcacaaac ttactgcaca aagaagcagg |
| 50401 | aaacatctgt ataaatgcgt ccacacagag cctgagtcca gaaatgaaaa gcagaagctg |
| 50461 | tgggagcctg tgattacaat agtcaggggc aaagaggtat cttcctttcc agcttgagca |
| 50521 | ggtccccata taacgtacca ctagggctgt ttattcggca tctgttgggc gccaggcaca |
| 50581 | gtgcttggtg ccttacccat catttaattc tcacttactc ccggcaaccg cctgtaatat |
| 50641 | aggtattgtg ggggggtggg gggggtgggg gggtggggaa tgaagaagat actttgcctg |
| 50701 | ggtcactagg attatttact tagccctatc tctgcagcta acactgtggt aaggaaagag |
| 50761 | gaaactgaaa tagaaataaa atcctggccc tagctcacaa tgaacttaca atacaagtac |
| 50821 | atcctttagt ggtttatata aatttgttcc aaaaaggaag actgtttcat tcaatcttta |
| 50881 | caagtcaact ttggaactta tccctggcaa gtcagaattt aaatgcatct ctaatatgca |
| 50941 | ttggatttga aaaaaaaaaa aacttttgtt ttttttttgc aaatagatag aagtaaatta |
| 51001 | taactcagtg ttccacaaaa caaacttgtt tggaaaagaa ttttaaactt ctgtgattaa |
| 51061 | attagaccca ggactaattg agtacacaga aaaacaagaa aatataaata tcagaaggga |
| 51121 | tttttttcta gttgctttct agttgtttaa ttgtgataat ttaattatca cattcaattt |
| 51181 | ctttccttta taaaaccagg ttaatattaa atttcaagca gagaaactaa gaggttaaac |
| 51241 | tggactcagc catgcatata gctgtaattt attacaggtg atcataaaca cctgtaataa |
| 51301 | aactctacaa aatggagaca aagaatctca gaatcatttg agctgagtta gaggcagttg |
| 51361 | tcagtcaatt ctaatcagaa acagataaag agtggaatag caccctgata gggaatgaac |
| 51421 | aagaaatagt ccaaggtgac agccatgtaa tcttgagtga gttaactctt ctttccatgg |
| 51481 | atcttctttt gaatcagaag gttgaaaatN ccaatatcta ctcctNcata attcaacagg |
| 51541 | attatacaaa actgtcagga gaaattagct gatgtattag atgtaaaagt aagttaagtc |
| 51601 | ttagtagaaa aggtaccaaa aagcatctgg cagaggcaca attctgagga cacatggtca |
| 51661 | aaaaaggtcc tttgcttttt gcgtgtaaaa tctccaaatt ctgtaagctg gttcatccta |
| 51721 | tttgcatgca aactggctgg gaatgaaatt agggcagRat gttatttgct catgttttaa |
| 51781 | ccattctttt ctcacagcct cctcctccta tgttatttac actgtttcct gccctgtgta |
| 51841 | ttgtttccag gacattcatt agattcaggg aaatgaaatt taatagggat gtctaatacg |
| 51901 | taattcaaga tttaaaaagg aacagaaaga tgccctggat tgacctaaca aattgttccc |
| 51961 | ttgactttcc tcagScgaag aaagaagaat tattaccaag aaaatgatcc ttatacacac |
| 52021 | cctaacccta ctctgcagtt tatgcatatc ctcttgaatc atgcatcagt tgtcatcaga |
| 52081 | gacaccttgg agtcccaggg gacagtaaca gcatcatggc tgggatcaca gcaaacccca |
| 52141 | tggggctaag tttctgagat ccaggcccaa gaaataccag cgtgggaagt aaaaataact |
| 52201 | catttttcgg accttaataa ctttcatggt ttgatttatg acaaggaaag aaaagtcatt |
| 52261 | tcctctacca agacctcaat acatggtatg ttcaaaacca aataaaaatg aaaagaaaaa |
| 52321 | aaaagagaag gaaaaagtat aatataatca caagtgacaa aaacgcagaa gagggggtca |
| 52381 | cagagatgtg atgtaagaaa aacttggccg ggcacgatgg ctctcacctg taatcccagc |
| 52441 | actttgggag gcggagatgg gtggatcacg aggtcaggag ttcgagacca gcctgaccaa |
| 52501 | catggagaag ccccgtctct actaaaaata caaaaaaaaa aaaaaattag caaggtgtgg |
| 52561 | tggcaagtgc ctgtagtccc agctactcag gaggctggga caggagaatc gcttgaacct |
| 52621 | ggcgggcaga ggttgcagtg agctgagatc atgccattac actccagcct gggcgacaga |
| 52681 | gcaagactct gtctcaaaaa caaacaaaca aaaagaagaa gaacccaacc tgccattgct |
| 52741 | ggtttgtgct ttattacctc tgctcataga agccatgagc catggagcgt gggtggcctt |
| 52801 | tttaagctgg aaaattccag gagacagatg ctagactcca aaaaggaaag gaaacacctt |
| 52861 | gattttagcc catgagactg tgttgaactt ctgaccttgg aacttcaaaa taataataat |
| 52921 | aatgaatttg tgctgctcta agccaccaag cttatggtaa tttattatga cagtgaaata |
| 52981 | aaacaaacac atctgaataa aaatctagag tacttcaaag taaaaaacaa aacaagtcga |
| 53041 | atattaacta tgctccagtc tcaaagagat catacagtta taggggatgt tttgcctgga |
| 53101 | atgcctggga ccagcctgct ccggattaac actgacgctg gagctcaact ccccagcatt |
| 53161 | ggcctctaac tgcatctctt gggttttctt tagactattt ggattaatgc atgtacacta |
| 53221 | tatacttttg gggcctcttc tgggatctgt gatataaaat tggggcaaga atataggacc |
| 53281 | tgtttccaaa atacttttct tagagaaact ctgaaacaag aagacaaatc ttttactatt |
| 53341 | ttaatagtgc catgagttag aaaataaatt gctcctccag gacttttcct ctgcccatat |
| 53401 | atgttctggc ccttgacttt ctggtttgtc cagacagtgg gtcagtccca cattcagaac |
| 53461 | cctgaggaat aaatYcaaga ttcctgaaga tgccaaggga cagcttggaa tcttgtagtg |
| 53521 | cgggtttact gagcaattta ggcgactgtc ctcagagtcc aattccttta ccatagacga |
| 53581 | tctctctgac tttgccagag catttcctca ctgtgtagta ttgcacctgc cttttgtgtg |
| 53641 | agatgctcta acactactat ggaacatacc ggacattcag tcactgctgc taggtccaca |
| 53701 | gccaactcac aggacttgat caaatcctct atcactgcca aagcttgttg tagttcagtt |
| 53761 | gagaatgctg aatctttggt tctctttggc ccatcctgtg gaaaacaaat gccttgttgg |
| 53821 | aaatcaaaag tataccatct gcaaagtgaa gaaggaaaac aggacagaga tgaggagcca |
| 53881 | gggatccagc caaccctgac tgagaagggt ggatattgat ctgtctccct gctcccaaag |
| 53941 | catggtggct aaagatgctt actgatttag gtatacggca tagagacaag caaaatcatc |
| 54001 | tacctctgtg cattgtacta gtgacgaatg aagttcataa atcatggaaa ttatattcct |
| 54061 | gctacataag aaacatatag aattcagcta tatagctgaa ttctatatgt ttcttatgtg |
| 54121 | tatatatatt atataaatat ttatatagta tattaaatat ttatatatta atatataatt |
| 54181 | atatgtttat atattaatta tatatattaa atatttatat attatatatt atataaaata |
| 54241 | taagtatata tattatatat atatatatat atatatatat attttttttt tttttttttt |
| 54301 | tttttttttt tttttttgag agcgagagag agtttcactc tgtcacccag gctggagtgc |
| 54361 | agaggcacga tctcggctcg ctgcaacctc tgcctcctgg attcaagcaa ttctcctgtc |
| 54421 | tcagcctccc gagtagctgg gattacaggt gtgcgccacc atgcctggct aatttttgta |
| 54481 | tttttagtag agacggagtt tcgccatgtt gaccaggctg ttctcaaact cctaacctca |
| 54541 | ggtgatccac ccaccttggc ctcctaaagt gctgggatta caggcgtaag ccactgtgcc |
| 54601 | tggccagaat ttagcttttt taaagcatgg aaaaacctag ctccttttaa ataggcttct |
| 54661 | ctctctttta tacccctatt ccctggggct tactgaaaaa aaaaatacca tatggccggg |
| 54721 | cgggtggctc atgcctgtaa tcccagcact ttgggaggct gagacaggca gagcacttga |
| 54781 | ggtcaggagt ttgagcccag cctggctaac atggtgaaac cccgtctcta ctaaaaatac |
| 54841 | aaaaattagc ttagtgtggt ggtgggcgcc tgtaatccca gctacttggg aggttgaggc |
| 54901 | aggagaacag cttgaaccca ggaggcggag gttgcagtga gcccagattg tgtcattgca |
| 54961 | ctccagcctg ggtaacaaga gtgaaacccc gtctcaaaaa aaaaaaaaaa aaaaaaaaaa |
| 55021 | aaagtagctg ggtgtggtgg tgtgtgcctg taatcccagc tactcaggag gctgaggcag |
| 55081 | gaggattgct tgaacacggg aggtggagtt tgcagtgagc caagattgcg ccactgcact |
| 55141 | ccagcctggg caacaaagtg agaccttgtc tcacaaaaaa agaaaaaaga aaaagaaaaa |
| 55201 | gaatacaatc tgagtatcat attcctatat ttaatgtgga agccttattc ctgtgggaaa |
| 55261 | aattatactt aaaatcatcc cagaggagga catttgtaaa ctcctataga gacaaagaac |
| 55321 | tccatagagg ctgctaagtg gaaatttact aatgatttac atgtaaaagc tataacatca |
| 55381 | tatttccaca ctgaatctcc cccaactctg tccttccttc ctccacttgt ccctggccct |
| 55441 | ccccacattg caccaccatt aaagatgcca aagagataag ccagcgctct gcacctcccg |
| 55501 | aacataaaga ctcagcattc agcRgaaaag cagtaactaa ttaaagagac caatgttcca |
| 55561 | attacaacca cagtgacatt taggatgtga ttggggtgat tgtttcagct ctaaaggctt |
| 55621 | ttgcatgggc ttgaggtatt ttatctccct gctacctaca tgctgtattt atctgttacc |
| 55681 | tggtaaatac acataaaaaa attttggttt attcaatgta tttttttaac atctcaagtt |
| 55741 | ctgcagtgaa gaacagctag cccctttgct gctccgcatc tggccctgac tctttttgtc |
| 55801 | ctctacagca caatgtagtc acagggttta atattttctt aatctatgcg gaagcactgg |
| 55861 | gtatttgcat ctttggatga gagacatgag tgacagggct gatgaatgga atagatcgtg |
| 55921 | ctgctgcctc tgaaaaacgt atcatggatt cgtagcttct cattcataat aaagaagctt |
| 55981 | cgcttcctgg taaaagaaaa cagactctca cctgaattta ttttaacaac atcaataagg |
| 56041 | gattaagagt tcttggcagg gcgcagtggc tcacgcctgt aatcccagca ctttgggagg |
| 56101 | ccgagggggg gagtggatca cgaggtcacg agatcgagac cagcctgacc aacgtggtga |
| 56161 | aaccccatct ctactaaaaa caaaaattag ccgggcgtgg tggcgggtgc ctgtaatccc |
| 56221 | agctactcag gaggctgagg caggataata gcttgaaccc gggaggcgga gttgcagtga |
| 56281 | gccgaaactg cgccattgca ctccagtctg ggtgacagag cgagactctg tctcaaaaaa |
| 56341 | ataaaaaaat aagaaataat ttttttttaa agttcttata ccaggcatct tcctccttac |
| 56401 | caaacactga atgctcagtt tcctttcaga tcctcacctc ttcccatccc ccttccttcc |
| 56461 | gctaaccctc cttcacaaaa cacgaggtgg ctagggtttt gaactgtata atgtagggtt |
| 56521 | cagaaaaatc ctttaaaaca ttattaaact cctctaacta gggctagctc ctcttgctcc |
| 56581 | aggctgtaaa aatatgacct gtgtcctgag ctgcttctgt tttcaacagg tccttgtcat |
| 56641 | ccattctgag cagaaagggc atgcaaatga ctgccccata agacatgtct caagtgtttc |
| 56701 | ttgctaaaac cagaatattc tataggaaag ggaagagaaa ccgcactgct ataMcacaga |
| 56761 | tttcttccta acctggtgtg gtcatggtca ccatttattc taagggaact ttggcagact |
| 56821 | cttagagttc acacacacgc acacacacac acagaggaga acaaggcata acatatagaa |
| 56881 | attagatata ctaagaggta caaagaagaa aacataatcc taccattcag gaaagccaca |
| 56941 | gcagacattt tatcatatct atttacttcc agtcttttat gtatgcattt tacgtatttg |
| 57001 | cttaattttg ttcccattaa aatttttttg gctgggtgca gtggctcacg cctgtaatac |
| 57061 | cagcactttg ggaggccgag gcaggtggat catctgaggt caggagttca agaccagcct |
| 57121 | ggccaacatg gcgaaacccc gtctctacta aaaataaaaa aattagctgt gcatggtggc |
| 57181 | gggcacctgt aatcccagct acttgggagg ctgaggcagg agaatcgctt gaacccggga |
| 57241 | ggcagaggtt gcagtgagcc aagattgcac cattgcactc cagcctgtgc aatggagtga |
| 57301 | gactttgtct aaaaaaaaaa aaattatgtg gaaggaagaa aatatattac cacttccatt |
| 57361 | tgggctgcaa atccctactt taaaattgcc tttaattttt ttgttttttt tcttgactgt |
| 57421 | gagttatagg atacatacta ttttagaaaa tctagataat aaagaccacc aaRtaaaatt |
| 57481 | gctataaatc ctccgaMcca tagacaaaca ccattaatag ttagttatat aaaatgtaat |
| 57541 | atttaatatt gagtacctta gccgagcctc ctagttcacc tgggtctatc tccatatctt |
| 57601 | caggagactc gaRatccagc agaccctgca ttaacccaaa agtcttgttg aaaattaaaa |
| 57661 | tgtacattgc catcaaaatg tagattgtca acaaaatcta ctgtgcaaac tatctgccac |
| 57721 | caatgctaag ctgactgaga acgtgtgtaa atgtgtaaga gggaaagaat aaatgatgta |
| 57781 | tttggcacag ggtcctttcc acaaacctag aatcatattg tacacactat tttgtagact |
| 57841 | actttttctc attcaaccat acctcatgaa tattttccca tgatattatg tttttctaaa |
| 57901 | atatgggctt tgattgtggt agagtgttat atcttacaga taaaccattt ttctcattca |
| 57961 | gcaatatctc atgaatattt tcccatgata ttatgttttt ctaaaatatg ggctttgatt |
| 58021 | gtRgtagagt gttatatctt agagataaac catttttctc attcaacaat atctcatgaa |
| 58081 | tattttccca tgatattata tttttttaaa acatgggctt tggttgtggt agagtgttat |
| 58141 | atcttataga taaaccataa tcgaattata gtccccctta tcattaaaca tttaggttca |
| 58201 | acaatatttt ttactataat aatgataata acttttgtgc atttttctta aagctaaatt |
| 58261 | cctagagtgg aattgtaaat gttaagagtt ttgaggcata tttccaagtt cttcagagaa |
| 58321 | ggactgtctt cctgccagct gtataggaaa gcctgtctca ccagccccta gcctacaagg |
| 58381 | ggtagtacca tttaatcaaa aatctttacc aattgaatga aaagagaaat cctacctcac |
| 58441 | tgttgtttta atttgacttc cttagataac tagaggtgta gaactttttt aaaaaaacgt |
| 58501 | ttgctactag tgtttattct tttgcaaatt gctatgtcct ttgcgcatct ttttattata |
| 58561 | atttccaaga gcacatgatt cattaagcat actgatcatc tgttacatat tttgttcaca |
| 58621 | ttttccccaa tgtttcatga gtctgtcttt aatggtataa gctatcactc atcaccctct |
| 58681 | ccccaagatc ccatgatcct ttctaaagca tgaggcaatc agtccaacat tcatgctctt |
| 58741 | tcaagccagc acatgtgtgc gagatgcaaa ataagctctg cccctgggga tagagaaggt |
| 58801 | cctagatagg attacaaggt Ygttcctttc tttgcaggca cgtaatggcc tgagctggtt |
| 58861 | tcacaggcac cagcaagctt agctgtgggg acactgctct ggtctgcctt gggtagctcc |
| 58921 | cacggctcct cacagacccc ccgcaaaata ttttgtagta attctatctc ttgtttcttc |
| 58981 | agtgaaaggc agctaacaaa accttctaag tccttttaac ctgtattatc tcatttaatg |
| 59041 | ctcccaacaa gcataccagg aaagtactat tattatctta tttctatagg tgagaaatct |
| 59101 | gaggctgaga gaggttaagt aacttgcctg agttcacgca gcctggaagg gtcagagcct |
| 59161 | ggattcagac ccaggttgtt aaactctcga gtctgtgttt ccaatgacga tgccccacag |
| 59221 | ccctctgcgt ctggtaccga acctagctcc tatgtaaatg tcacctctgt gggaaagctg |
| 59281 | gaacctaggc tgagggagga aggaccatca ctttcgtcct gctcatgcct cactgggccc |
| 59341 | agaggttgga cttctacaat aggaatgaca gtgacaatgg gacatgcaga ggagcatgag |
| 59401 | gccctgagtg agtgctgggt caaacatgct ctgaggtgct gctcgctgaa agaagcagat |
| 59461 | cattatcctc atttctatgg tggtgtgaca gtctcaatgt ctaagataac agctacacat |
| 59521 | catgcagagc ttcctgggct gtgccaagcc ctccgcatgt gctaatcatc tgcttcataa |
| 59581 | cgatcttatg gaggaggttc tagtaccctc attttacata gaggaaaact aaggcccaag |
| 59641 | aggKcaaggt ctcataactg acaaatggca gagtacaccg aggctgaaac tgctaatgga |
| 59701 | aagcttgaaa ggagtaactt tgaaaaaatg tttttaaaat tatttttaca gatgaggtct |
| 59761 | tgctatgttg cccaggctag tcttgaactc ctggactgaa gggatgcctc ctgcctccac |
| 59821 | ctcctgagta gctgctggga ctacaggtgt gtgttatcat gtctggcctg aaaggggtaa |
| 59881 | ctttttctga ggggagactg ttgggcaatg agcttaacct tcctgagcct cagtttcttc |
| 59941 | acagtccaga gcatggctgg tatgtctgat tgtggagatt aaataagaaa tactgtaatc |
| 60001 | ccagcacttt gggaggctga ggtgggcaga tcacgaggtc aggagttcaa gaccagcctg |
| 60061 | gccaacatgg tgaaaccccg tctctactaa aaatacaaaa attagccagg catggcggcg |
| 60121 | cgtgcctgta atctcagcta cttgggaggc tgaggcagga gaattgcttg aacctgggag |
| 60181 | gcagaggttg cagtgaactg agatcgcgcc attgcactcc agcctaggca gagtctcact |
| 60241 | ctgtctcaaa aaaaaagaaa gaaagaaaga aaaaagaaat acacgtgggg aagtacccag |
| 60301 | taaacagtag gtgcttgttt ttgtttttgt ttttgttttt tattaagaaa tagtggccac |
| 60361 | tgggcaagag ctacctaaat gccttataga ggttaattca acagacaact tatcgtggtg |
| 60421 | gcccaaacct ttctctcaag ctcacaccta gaaaaccagc catgataggc cctcatatcc |
| 60481 | tgtttggagt gtctgagaat gcttgcctgc agatgtgcta gtgtcaatga cttatcacta |
| 60541 | aggaggggta aaggtcagag agagagagag aaaaccactt aggggttcag ggttcagata |
| 60601 | tagcaaagtt cattctgtta ggctgctctc agggtaagat cctaaatcta agaatggagg |
| 60661 | tcagtgctgc aaaaggatag aagacttcag ttttgtcttc tcccttccac aagttaaaat |
| 60721 | gcccataaaa cagtaacttt ggggaaaatc acactctcgc tcccaaagag ctctcttccc |
| 60781 | ctaagccaga ctccttagtg attcatgccc tgagctagac attggactag tgaggccctc |
| 60841 | aggtgccctc caactctatg attctttggt cttactccac attgaagagg gctgatttag |
| 60901 | agttggagga aagaaaaagc ctgcaggata gacgtatctt cagccttaga agaccaaggc |
| 60961 | acagctctac atgtgtaacg acatgggcac cacagtgcag gcgtctggtt tcccaccaag |
| 61021 | gaaaagcact gtctcattga ttttaccttc aacagcatct ggtattttat aagtctctgg |
| 61081 | cttggtcctt tgagatactt aaaaagaggg agattgtgat ccagttggcg ctggcatacc |
| 61141 | tttaaaagcc aaatagaaac aacataaagc aaaacaaaca acacacacag aataaaagtg |
| 61201 | gcagcattta ggtacaaaac acaaaattgc ttttcctgga caacaggggc agctgtgtcg |
| 61261 | agcagctcca taaggctgtg gggttgttct ttcccctagg aacagtgtgt gaaataagag |
| 61321 | aaagaggaaa gaggacggct cggctaatat ttttacaggc atctagggaa gcagaaccat |
| 61381 | ttatttcttt tagaaagcaa gttcactaga agacagtggg gcatttcaag tccttctgaa |
| 61441 | aaagaggcca cctatcttca agttgggggc tgccagagtc tattcaggcc ttttggagcc |
| 61501 | tgaaggatac agacggtttg tagacatcat tccagggagc ccaaagtgtg acatttttta |
| 61561 | cttagtttca tttttaaaac ccatcttaga agaagacact ggttttctca ccagatatag |
| 61621 | gtgctcctca caggcctatc ccattacaac ttttcatgtg taatttcaaa gtatgggaca |
| 61681 | ctttaaataa acataaatcg caatgccaag ttcctctccc tagtcccact tttgatttct |
| 61741 | ttaacaactt gatgcataga aacattttcc aggaagaagt ttgtcttctc aacattaccc |
| 61801 | caaagtaggc gcagtcttga cactcttgcc agattgccct agctcggggc agattcttat |
| 61861 | ggtatttaaa atatatctga agatcttctt tctaggtggg aaaaaattag aataaattaa |
| 61921 | tcaggcagca tttttcagta agtggacagg atgatacagt ttattcattc attcaacaag |
| 61981 | ccagagtatt ctaatgtgta aggaattggg ggtgtacagt aagtaatctt atgttgggat |
| 62041 | gtgtgtgtga tggagagata aacagaaggc tttaaacaac cataactacc agacatagat |
| 62101 | aggcacagag tgctctggga gcatctacaa gggctattgg taaatacata agtagactca |
| 62161 | caaacaggaa ttggttttta tctttatcat tcttttcctg ccctttgtcc aggacatcga |
| 62221 | catctcccca caacaggtct gcaatgagta gtaaaactga cagaaaagag gctggtgttt |
| 62281 | gatggtcccc ctctcttctc tctccaccct ctccttgtgg tcatcgtgac accaaccctg |
| 62341 | caaagaagcc caagaaacaa ccaaaagatg gaaacatgcc agaggggaga tgtggtagat |
| 62401 | cgggcaacca atacggagct cagtcatttg ggcaaccctc aaagtgacag gctccgcctc |
| 62461 | aaacatcact ttcaacctgc agcacaagac tctgagacag gaaatgttat gtgactgttt |
| 62521 | cggagctaga gagcaaggag gactcaacgt gctgttcaac ctgtattctc atggtatcaa |
| 62581 | agacgctgag aggtgggcac atatctgcca atctcccgga gagaaagcac actccaagcc |
| 62641 | tctggcgtca gcttgccagc ttcactcgct ctatcaggga gcctggccca cagcggctgt |
| 62701 | gctggccaca gatcccatgc cattctggtg gagaagccca tgtagccacc tccttgcaga |
| 62761 | agtctgtgtc cctgtcctcc tgctcagccc aactctggat ttccttcatg tcaacacaga |
| 62821 | tgattccttc cctctcagct tcaatgatgc ctgttctact cccaaataca ttgctatagt |
| 62881 | ggatgacttc catttctgta gctctttctt tctcccaata ggttgtaagt ggtcatttca |
| 62941 | gcataggaac tacagcaacc gtcctttgct attccctcca ggaaaaaaat ggcacacaca |
| 63001 | tttaagacag atggcccttg aaaaatgttc agactggtta aggagctaga ataataactc |
| 63061 | agagcttaac aatatagtat tgacctaaat tgctcttcta aaggctaaca gtccaaaatt |
| 63121 | cctcagagaa aggatgtcca ctaaccagca acatacaaaa gtgcccaatt ctcctaatct |
| 63181 | ccttatagaa tgaatcagga ggctataaaa ttgctaccag aaactcatat tcacacaaga |
| 63241 | atacatactt gcttacggat taagactgcg gggctctggt ggcggatggt ccaggttcag |
| 63301 | ttcttggctt tgccaaatac aaatcatatg gcctaaagta agttacctaa ccttcctaga |
| 63361 | cttcagtttc ctcatctgtt aagtgggccc ataacagtag tcaactcctg tgactgttgt |
| 63421 | gagattaaat gagatgatgt atagaaacct ctccacaaat gcccaatgga tagagtactc |
| 63481 | aaaaaatggt ggctattgtc aaagcacttt ttactttatg gaatcctctg catacattat |
| 63541 | tttgtttccc acaacaactg tacatattgg tactattagc ctgttgtgtg gaggaggaaa |
| 63601 | gtgagattcc aaagtttaag ttatttactg gaggtcatgc agatagcaag tggcttgacc |
| 63661 | aacatgataa atcgggtttc ctgacttacc agttactccc acatatcagt gtgagcaccc |
| 63721 | attccagtgc tctttccatt ttaccatgtg agatctcaga gtacttggaa cccacagtac |
| 63781 | actatggaca aatattattc tatatagaac atggtctctg gaggagaatt gtttcagaag |
| 63841 | atacaggcta aagaggctga ttaaaacact tggtcagaaa actatttcaa aattcattat |
| 63901 | cttggaaggc cgaggtgggc ggatcacgag gtcaggagat ggagaccatc ctggccaaca |
| 63961 | tgatgaaacc tcgtctctac taaaaataca aaaattagcc gggcgtggtg gcacatgcct |
| 64021 | gtaatcccag ctacgaggga gggtgaggca ggagaatcgc ttgaatcagg gagtcagagg |
| 64081 | ttgcagtgag ccgagatcac gccacagcac tccagcctgg tgacagagcg agactccgtc |
| 64141 | tcaagaaaaa aaaaaaaagt actacttgta ttttgtctct aacatcataa atcacatagg |
| 64201 | gctaagtcag tgttttctgg ctgggttaat gatttacaat gttcctccca acatggcggg |
| 64261 | gcgctatcaa aaaactttgt aaaactttac aatttaggag aggaattcag aaaaggtatt |
| 64321 | tagttaataa ttctctgaca atttcctcat atttgaacaa tttgataata tccactttcc |
| 64381 | cacaggaact ttgcccattt ctcattgagc aatttaaata atctttgcag taatgacata |
| 64441 | cataaccata aaatacttct aatctctgaa actagatttc acattagtcg tttgttaagt |
| 64501 | gcaaaaattc aaaatagtat ataaagcact aataatgtaa tagttcaaaa aaattaatga |
| 64561 | atcagaatga gtataaaata gttttcagtg tacattgaat tctggtgctt tgacacaaag |
| 64621 | cctatgatga gttgacatgg aaagccatat ggggccacca aaaggctctg agcatggaag |
| 64681 | attccttctc tccattaatg tcaaatatct tgaaacctgg aggctttgtt tgcttccagt |
| 64741 | tatcataagt attgctctca gcctcagtca taatatgagt tgcttacaac tttaagttgt |
| 64801 | ttgatctgtt gccttgggtt cacaaaaata aaaataagaa agagaatgag aaggactagt |
| 64861 | tgagaaagag aggagaaatg agaactattt agaaggcaca gggggtcagg atcatctcca |
| 64921 | tggtcgtgtc ctagaaagta cctggtgggg attgatgttg ggtggaacat ttctctgtgg |
| 64981 | cagctcccac tggcccatat agcctatttg ttgcctaaaa cagggtggag aaacagggaa |
| 65041 | agagggaaga agtctgggag gtgggaagaa ggggtagttg gaggtataat gttgtaggcc |
| 65101 | aagatgttca taaattgagt actaagccta aaggaggctg tagatttaga attttcgaat |
| 65161 | catcctctat gttgtaaaaa atagaatgga aagaaaatgt atttcctgag gtttttagtt |
| 65221 | taacaccagt gcttctcctc ttgagagcat tagtacagct ttattgtggg actctaacct |
| 65281 | cgctgtatga ttcttgtttt aaaacgtagt tgagtcattc agaagaggat cctctgcttt |
| 65341 | ggggaccatg gatggccagc cttccatttc atggggcttt gcaggaagcc aggcttgcgg |
| 65401 | ttactcccac atatcagtgt gagctcagtc aaattggact cattctcacc ttggataaag |
| 65461 | tcagataaag ttgggaggcc aactcatgtt ttccattcaa agggcagata caagatatac |
| 65521 | tgtcttgaca aattgcaagt gtacagtaca gtattattcc atgatagtcg ccatgctgtg |
| 65581 | cgttacatct ccaggactta ctcatctcat aactgcaagt ttgtaccctg tgaccaacat |
| 65641 | ctccctactt cctctagact ccagcccctg gcaaccaccc ttctacttca atgagctcaa |
| 65701 | ctgtttttgg attatatata tatacatata cacacacaca cacacacaat ggtaatgaat |
| 65761 | gtattcatta acttgattgc agtaatcatt tcacagggtg taagtatatc aaaccatcat |
| 65821 | gttgtatagt ttgaatatat gcaattttta tttaccaatt atacttcagt aaagcttgga |
| 65881 | gtcggaggga agcagatatg tttttagacc attgatgttt ccatttgtcc ttgccactta |
| 65941 | acgcagaaat gtaagggctg actttaacat gattatttac tgggagtaca gacttactct |
| 66001 | cttgagaaag caatgtgcca gaagttcagg gttctcagca cacttttcca actcttttag |
| 66061 | aaaagtccta gaaaaataaa agtatacaag taataatgtt ttgattttga catgtaggta |
| 66121 | attaattttt taagtacaat cacataaggt ttcaagaggt gcctagaaaa atgttctcca |
| 66181 | ggccttcttg ctcttatctt ctacacattg attcagRaaa aaaagctgta ttctgaggta |
| 66241 | ttactacaat taccctgaaa tcactcacaa ctttaagttg ttcgatctgt tgccttaggt |
| 66301 | tcacaaaaag aaaaataaga aagagaatga gaagaactag ttgRgaaaga gaggagaaat |
| 66361 | gagaactact cagaaggcac agggggtcag gatcatctcc aattatcaga acacagctgt |
| 66421 | tcgggtaatt ctaagcacta gtcatagata gactggatac attcactgat gctctctctt |
| 66481 | acatgaaagt aacgttcata agaactgctg ctgctgctgt tgctattact aatatcattg |
| 66541 | acagcttact ataagccagg cataagctaa gtgctccgca aatagtatct catttaatcc |
| 66601 | tcaaccaaag atagggtttt ataaatataa aacctggggc tcaaagaggc tacataactt |
| 66661 | ctctggggaa atcaacagaa atgggtttcc atttcagttt tgcgtctgac caacggacga |
| 66721 | ggaatggggt taggaagcaa ctgtggttca attcaccaag cagcttttct ccctctgtga |
| 66781 | attaggtgtg caatcttggg gtcatcatag cgaataaaga gagttaacaa gttaccattc |
| 66841 | ccatatgtgt aacatgaaag ggtttgaaga tgatttccaa agtcccttat tcctgcaatt |
| 66901 | ctctagaact caatatccaa gcagccccga gttaaaagta caacttgttt gagcagtcag |
| 66961 | cattttctaa caccctagtt cccagcatcc ccttatgata gtagtatgga gatgtgtatc |
| 67021 | ttggtcatct ttgtattccc aatgctgagc acagtgcctg gcacagaaca ggtgctcaaa |
| 67081 | aaatgctgat gcatgaataa ataaacaaag gaacactcaa ctgcatacaa catggatgtc |
| 67141 | tgacactggg tccctgttcc tttgatgtct cttccctttc tctccaggga tcctgtgggg |
| 67201 | cttctcccct cctttcactc caacccaccc caatccaatc cactgactcc aaaccacttg |
| 67261 | cttaaacctg aaaacatcta agtgttttca tctctcaatt ccatcagtct catttccacc |
| 67321 | cttctSttac tctccattct ttctgataac atgaattatt atatacctgt tgtgaaattc |
| 67381 | gtaaagttct ctaatattcc caaagagaaa gtccttgtta ttctgaagaa catctggaat |
| 67441 | tagatgcttt agccaaataa aatccattgg agtgatatat ccctgcagag gtgcaaacaa |
| 67501 | ggtaggtgtt acaaacagga acataccaga gatcatctga attagctact gcaaaaccca |
| 67561 | aaactttagt gaaatgattg aagaatatag cacacttgtt tcttgaaagc tatcgtattg |
| 67621 | agtactttct tttgatatca ttttcctcat gtgcccactt tcagaaggag atggcttaat |
| 67681 | ggcaatgatc ataaaatggc atttttgtgg gaagaacaaa tatatttaaa aatgtttatg |
| 67741 | ttacacaggt ttaatctaaa agaagggaaa atgtacccac tgttttatga actaatacaa |
| 67801 | ttactattat tattatgatt attattaata ttgagacgaa gttttgctct tcttcccagg |
| 67861 | ctggagtgca atggcgtgat ctcggctcac tgcaacctcc gcctcctggg ttcaagtgat |
| 67921 | tcttccgttt cagactccca agtagctgga attacaggca cacaccacca cacctggcta |
| 67981 | attttcgtat ttttagtaga gacagggttt caccatgttg gccaggctgg tctcaaactt |
| 68041 | ctgacctcag gcgatccacc cgcctcggcc tcccaaaatg ctgggattac aggcgtgagc |
| 68101 | cactgcatcc ggcccaataa ttatttttta atatctgaat aatgtttact cagcatatat |
| 68161 | atcatagttt actaaacatt ccctttttat ggacatttgg gttgcttcta atattttatg |
| 68221 | gtaaaaatgt gattataaat aatcgcgtac atataatttt ttccttaaga ttattccctt |
| 68281 | aggaaaatag ggaaaacatg catttttact cttaagaaaa agactacttt aaaaaaacaa |
| 68341 | gaaataatct tcccttatta aagtatcagg aacacttata atatcctttg ttggcaagaa |
| 68401 | gatggtaaac ctatttgggc tttgctggaa tcagtgtgaa ctggcttaat tctttggaaa |
| 68461 | ctatttggct acatggatca agaaaccaca gggatgttct tattcctact ggagacactt |
| 68521 | gtaatattct aagacctgaa accttggtgg gaattctgga gacttctggc aactattttg |
| 68581 | agtctctttg tacaacaaaa catctgcagt ttgaatatat gcttgatacc ccactcccca |
| 68641 | aatctactta catcaattat gcttttaatc tcttttatgt aaatctcttc agtctcaagc |
| 68701 | aagtcacgta taatgcgcct gaatcacagc agcaggtggg agggtgaaag agagagagac |
| 68761 | agggagagga gaatgttctt ttagaattct gttagaataa tgctcatcaa tacagttccc |
| 68821 | ttttagtggc tcacctactt agtttctggt cagttcattc tgttctattg aacacccaag |
| 68881 | gtcagcatct cacaaacgca cactgtgatc acactggtat caagaagaaa cagcaaggtt |
| 68941 | agagaactca aaatccttta ggcagccagt gaatgacctg tcctctgggt gggccatata |
| 69001 | gtgtgggggt caagaaggga gatgttggag tcagaaatac caggtctgga tccttagtca |
| 69061 | aggaaggtct gcctgacccc acccccaccc cctatattgt atacattgta taagtggttt |
| 69121 | cctagatttt ctctccagct ttctgctcac atcaagcttt cttttccttt taagagaaag |
| 69181 | ggtcttgccc tgttgcccag gaatgagtgc agtggcatga tcatggctca ctgtagcctt |
| 69241 | gaactcccgg gctccagcaa ccctcctgcc tcagcctccc aagtagctag ggctacagat |
| 69301 | atgcaccatc acacccagct aacgcctttt tttttttttg gtagagatga ggtcttgcta |
| 69361 | tgttacccag gctggtctca aactgctact ttcaagcaat cctcctgcct ttgcctccca |
| 69421 | aactgctggg attataggtg tgagacactg tgccaggcca gatccagatc tttgacccat |
| 69481 | atggatgtat tttgttgtac atggggttag atgtatgcta gctccttcct ctggcattgg |
| 69541 | atgtttcact gtgatttcct aaggcaaaag atatgacttc tgtctgcttc tgtcaaggaa |
| 69601 | tgcaggtgga agatgtggtg gacagaattt aagacggtcc ccaagacttc tggccccact |
| 69661 | gcacatacgc ctcttccaat caaacactaa tttaggggat gctgtggaag gattttgctg |
| 69721 | ttcatttgat aggtaatcca gttgggcctg tcctaatcat atgaactcag atctatMtgg |
| 69781 | gtcaagtggt cagagactgg aagcataaaa aagattcaac acaaaggaga ttcccctttg |
| 69841 | ctgacttttg agatggaggg ggccagatgg aatggaatgt gggcagccgt aggaactgag |
| 69901 | agtggctccc agctgacagg cagcaaggaa gggaaatcag tcctatagtt gcaaggaacc |
| 69961 | gagtcctgcc acaaccacgc gaacttagaa gaggatccag agctccacat gaaaacacag |
| 70021 | ccagccaaca ccttgacttt agccttgtgg gactcctgtc ccagggaaac tgtaggtctc |
| 70081 | ctaagtttgt ggtcagtttt tatatagcaa tagaaaacca gtagaaagag taaggccagt |
| 70141 | ccccacatct atcccagaca gactttcttt ctctcctcag gctacgtggc catatttatt |
| 70201 | atttctttta ggagtccaag tagatgagtg tttatacatg tgtccttgtg tagaatatat |
| 70261 | atatttattc ttgttaacta ttgatggatt atctattaaa tggcagcctc tcctagaaaa |
| 70321 | tgatttgttt ttctctcata aaaatggatg tgacaaatga tccaatagaa aaaaatggga |
| 70381 | aaggacacaa aataggagat tcacacaaat attcaaatgg atatgagact cgtggaaaaa |
| 70441 | atactcagta catacattca acgaaggcat attttaaaag agcaacaatt ttcatctatt |
| 70501 | agaatattaa acatttcaaa cctcatagaa agactggtaa atgagggtgt ggaaaaaccc |
| 70561 | tcacacaata ttggtaaaat gtgtaaattt gtgcaagctc tttggaaggt aatttagaaa |
| 70621 | catctcaccc aaatgtaaaa cacgtgctcc ttggttcagt aatttcactt ctaggaatgt |
| 70681 | tcattgttta taattaggac aatataactt caacaacaac cagatataaa ctggttactt |
| 70741 | aaatgctcat caaaaaatta tggtacatgg gccgggcatg gtggttcacc cctgtaatcc |
| 70801 | cagcactctg ggaggctgag gcaggtggat cacttgaggc caggaaatcg agaccagcct |
| 70861 | ggccaacatg gtgaaaccct gtgtctacta aaaatacaaa aaattagatg ggtgtggtag |
| 70921 | cgcatgcttg taatcccagc tacttgggag ggtgacgcag gataattact cgaacccagg |
| 70981 | aggtggagat tgcagtgagc cgagatcaca ccattgcact ccagcctggg caacaagagt |
| 71041 | gaaactccat ctcaaaaaaa aaatatatat atacatacat atagatatag atagatatat |
| 71101 | gtgtgtgtgt gtgtatatat atatacatat atatggtaca tacatataat gatgaaatac |
| 71161 | catgcaattg ttaaaaagaa tgaggctgac taaaacactg atatgacaga ggccaggtat |
| 71221 | ttttagtgaa aaaacaaaac aagacgtaga caagtaagca tagtatgact atttgtgtaa |
| 71281 | aaaatgaatg tgtatgtaga aatatacgta aaactgtata tgctcagaaa atttatgcaa |
| 71341 | agaaacttaa cacattgtta aaagtgattg cttttggaga gtggtactga aagctaaagt |
| 71401 | atgggaaagg agaatttcta catcttactc tatacccttc tacaatcttt tttaaagtga |
| 71461 | aacatttact gcttttataa tggaaaaata gggtttacat aatattttaa aatgaatgtt |
| 71521 | actggaggta atgatatgta aaccctattt gatagatata aataaataat acaattcaca |
| 71581 | tattttacag ctcattattc tttcttataa tcatttttgt gtctatttat aaatacttgt |
| 71641 | ataagacaag actgataaac aagatgtacc acagaggatg taatttccgg aagtctaaaa |
| 71701 | ctcccaaaaa gttacttaaa catcatactt tactaagtca ctgagaaaga ggttcctatg |
| 71761 | ccttttataa atcacccgga aacaacaacg gtttctatgt cagaaggaag tacaggcctc |
| 71821 | aataccaata tgtttctacg agccctggca tcaagtggga ccacagcaac agttaagcat |
| 71881 | tcatggaaaa cctgagtaca atcagcctct tatgccttca tgaagcactg ggcttaagca |
| 71941 | acttggcaag ctggcttcag gatgcattga caattaaagg aagtcagaag gcagaaataa |
| 72001 | ccaggaggtg gacagaagtc aaggttatta tccaaaaatg atctatgaac ttaagagact |
| 72061 | cttaggagct tttaggactc taatacagga aaatattcac atctctgaag gaaaagaaat |
| 72121 | cccttggtca tattctggat tacagtttgg aaaaaaagtc tagaaatctc atccaactgc |
| 72181 | ttcattttac ctgttggagt gcaagagata aaatgccaag gataggaact ccactgaaga |
| 72241 | atgtaggcaa atcaatattc attaaataca ttcaaattaa tatatgactt aaaaagagcc |
| 72301 | ctcacatcag tgttaggcca ttgcttttcc ttgcttctct tgttttcctt tcaaatacct |
| 72361 | ttataaaata ggagaaaatg tatgactctt tttttttttt tttttttttt attatactct |
| 72421 | aagttttagg gtacatgtgc acattgtgca ggttagttac atatgtatac atgtgccatg |
| 72481 | ctggtgcgct gcacccacta atgtgtcatc tagcattagg tatatctccc aatgctatcc |
| 72541 | ctcccctctc ccccgacccc accacagtcc ccagagtgtg atattcccct tcctgtgtcc |
| 72601 | atgtgatctc attgttcaat tcccacctat gagtgagaat atgcggtgtt tggttttttg |
| 72661 | ttcttgcgat agtttactga gaatgatggt ttccaatttc atccatgtcc ctacaaagga |
| 72721 | tatgaactca tcatttttta tggctgcata gtattccatg gtgtatatgt gccacatttt |
| 72781 | cttaatccag tctatcattg ttggacattt gggttggttc caagtctttg ctattgtgaa |
| 72841 | tagtgccaca ataaacatac gtgtgcatgt gtctttatag cagcatgatt tataatcctt |
| 72901 | tgggtatata cccagtaatg ggatggctgg gtcaaatggt atttctagtt ctagatccct |
| 72961 | gaggaatcgc cacactgact tccacaatgg ttgaactagt ttacagtccc accaacagtg |
| 73021 | taaaagtgtt cctatttctc cgcatcctct ccagcacctg ttgtttcctg actttttaat |
| 73081 | gattgccatt ctaactggtg tgagatgata tctcataatg gttttgattt gcatttctct |
| 73141 | gatggccagt gatgatgagc atttcttcat gtgttttttg gctgcataaa tgtcttcttt |
| 73201 | tgagaagtgt ctgttcatgt ccttcgccca ctttttgatg gggttgtttg tttttttctt |
| 73261 | gtaaatttgt ttgagttcat tgtagattct ggatattagc cctttgtcag atgagtaggt |
| 73321 | tgcgaaaatt ttctcccatg ttgtaagttg cctgttcact ctgatggtag tttcttctgc |
| 73381 | tgtgcagaag ctctttagtt taattagatc ccatttgtca attttgtctt ttgttgccat |
| 73441 | tgcttttggt gttttggaca tgaagtcctt gcccacgcct atgtcctgaa tggtaatgcc |
| 73501 | taggttttct tctagggttt ttatggtttt aggtttaacg tttaaatctt taatccatct |
| 73561 | tgaattgatt tttgtataag gtgtaaggaa gggatccagt ttcagctttc tacatatggc |
| 73621 | tagccagttt tcccagcacc atttattaaa tagggaatcc tttccccatt gcttgttttt |
| 73681 | ctcaggtttg tcaaagatca gatagttgta gatatgcggc attatttctg agggctctgt |
| 73741 | tctgttccat tgatctatat ctctgttttg gtaccagtac catgctgttt tggttactgt |
| 73801 | agccttgtag tatagtttga agtcaggtag tgtgatgcct ccagctttgt tcttttggct |
| 73861 | taggattgac ttggcgatgc gggctctttt ttggttccat atgaacttta aagtagtttt |
| 73921 | ttccaattct gtgaagaaag tcattggtag cttgatgggg atggcattga atctgtaaat |
| 73981 | taccttgggc agtatggcca ttttcacgat attgattctt cctacccatg agcatggaat |
| 74041 | gttcttccat ttgtttgtgt cctcttttat ttccttgagc agtggtttgt agttctcctt |
| 74101 | gaagaggtcc ttcacatccc ttgtaagttg gattcctagg tattttattc tctttgaagc |
| 74161 | aattgtgaat gggagttcac ccatgatttg gctctctgtt tgtctgttgt tggtgtataa |
| 74221 | gaatgcttgt gatttttgta cattgatttt gtatcctgag actttgctga agttgcttat |
| 74281 | cagcttaagg agattttggg ctgagacgat ggggttttct agataaacaa tcatgtcgtc |
| 74341 | tgcaaacagg gacaatttga cttcctcttt tcctaattga atacctttta tttccttctc |
| 74401 | ctgcctgatt gccctggcca gaacttccaa cactatgttg aataggagcg gtgagagagg |
| 74461 | gcatccctgt cttgtgccag ttttcaaagg gaatgcttcc agtttttgcc cattcagtat |
| 74521 | gatattggct gtgggtttgt catagatagc tcttattatt ttgaaatacg tcccatcaat |
| 74581 | acctaattta ttgagagttt ttagcatgaa gggttgttga attttgtcaa aggctttttc |
| 74641 | tgcatctatt gagataatca tgtggttttt gtctttggct ctgtttatat gctggattac |
| 74701 | atttattgat ttgcgtatat tgaaccagcc ttgcatccca gggatgaagc ccacttgatc |
| 74761 | atggtggata agctttttga tgtgctgctg gattcggttt gccagtattt tattgaggat |
| 74821 | ttttgcatca atgttcatca aggatattgg tctaaaattc tcttttttgg ttgtgtctct |
| 74881 | gcccggcttt ggtatcagaa tgatgctggc ctcataaaat gagttaggga ggattccctc |
| 74941 | tttttctatt gattggaata gtttcagaag gaatggtacc agttcctcct tgtacctctg |
| 75001 | gtagaattcg gctgtgaatc catctggtcc tggactcttt ttggttggta aactattgat |
| 75061 | tattgccaca atttcagagc ctgttattgg tctattcaga gattcaactt cttcctggtt |
| 75121 | tagtcttggg agagtgtatg tgtcgaggaa tgtatccatt tcttctagat tttctagttt |
| 75181 | atttgcgtag aggtgtttgt agtattctct gatggtagtt tgtatttctg tgggatcggt |
| 75241 | ggtgatatcc cctttatcat tttttattgt gtctatttga ttcttctctc tttttttctt |
| 75301 | tattagtctt gctagcggtc tatcaatttt gttgatcctt tcgaaaaacc agctcctgga |
| 75361 | ttcattgatt ttttgaaggg ttttttgtgt ctctatttcc ttcagttctg ctctgatttt |
| 75421 | agttatttct tgccttctgc tagcttttga atgtgtttgc tcttgctttt ctagttcttt |
| 75481 | taattgtgat gttagggtgt caattttgga tctttcctgc tttctcttgt aggcatttag |
| 75541 | tgctataaat ttccctctac acactgcttt gaatgcgtcc cagagattct ggtatgtggt |
| 75601 | gtctttgttc tcgttggttt caaagaacat ctttatttct gccttcattt cgttatgtac |
| 75661 | ccagtagtca ttcaggagca ggttgttcag tttccatgta gttgagcggc tttgagtgag |
| 75721 | attcttaatc ctgagttcta gtttgattgc actgtggtct gagagatagt ttgttataat |
| 75781 | ttctgttctt ttacatttgc tgaggagagc tttacttcca actatgtggt caattttgga |
| 75841 | attccctgct ttattattct aaagcaagct tgtccaacct gcggcctgtg gcccaacaca |
| 75901 | aatttgtaaa ctttcttaaa acgttatgag attttttttg cgattttttt tttttttttt |
| 75961 | tttagctcac cagctatcgt tagtgttaat gtattttttg ttttgttttg ttttgagacg |
| 76021 | gagtcttgct ttgttgccag gctggaatgc agtggtgcag tctcggctca ctgccacctc |
| 76081 | tgcctcccag gttcaagcaa ttcccctgcc tcagactccc gagtagctgg gactgcaggc |
| 76141 | gtgcgccacc atgcccagct aactttttgt atttttgtag agatggggtt ttaccatgtt |
| 76201 | ggccaggatg gtcttgatct cctgacctcg tgatccaccc tccttggtct cccaaagtgc |
| 76261 | tgggattaca ggcgtgagcc acctcgcctg gccagtgtta atgtatttta tgtgtggccc |
| 76321 | aagacaattc ttcttcttcc agcgtggccc aggaaagcca aaagattgga cacccctgtt |
| 76381 | ccaaagcatg taattttatt cacagaaaag actctcggcc gggcgcggtg gctcacgcct |
| 76441 | gtaatcccag cactttggga ggccgaggca ggcggatcac gaggtcagga gatgtagacc |
| 76501 | atcctggcta acacggtgaa accccgtctc tactaaaaat acaaaaaatt agccaggcgt |
| 76561 | ggtggtgggt gcctgtagtc ccagctactc gggaggctga gacaggagaa tggcgtggac |
| 76621 | cccggaggtg gagcttgcag cgagccgaga tcacaccact gcactccagt ctgggcaaca |
| 76681 | gagcgatatc cgtctcaaaa aaaaaaaaaa aaaaaaaaag actctcaagg atttaccatc |
| 76741 | taaaatcatc aagtgtatat catgcagatg aacaacagaa aacactggga gtattcaagt |
| 76801 | aattaaaaat aacctttcag caccaacagc aatttctgtc ctgatggcaa tctactcaga |
| 76861 | gctagaggac tgcacttagg ataccaaagg gagcttagga tgcaagagca gcctgcacta |
| 76921 | atgtactctg cgttagttta catccgggca ctttgcttta cgtttcaaaa cagacgccgc |
| 76981 | ccttagctca ttaaagggga aatggaggta taatgtgttt taaagggatg tttcttctct |
| 77041 | gaagccatat ttcagggtag catgaaaaca gagctttgga tatctggttc cattctacat |
| 77101 | gacacctcat gcttgtttca aatgacacca caaccaaggg gcaggaatga aaggaatatg |
| 77161 | ctaaaaaaaa aaaatgcaat cctttttagc aagaaagatc attaaggatt tcctgataag |
| 77221 | tttcttcttt gttcatgtgc cctcctctgc tccccctttg aactactgcc cctgtcaccc |
| 77281 | ccccttcccc gcctccccgc cgtttctcag ttctccaata gggatccagt ctgccagagg |
| 77341 | tctatttttg ctcattattg tcaacacaat ttgccatcaa gagttttcac ttactcctgc |
| 77401 | tatcctatca tcacattatg catgtcagaa agcattacag atttttactt ttcaagaacc |
| 77461 | cgaactcaga tttccaggct tctgtccttc ctgtttcatg ctgttgactc tcccacatct |
| 77521 | ttgtcttcct cctagacctt tggtcactct tgagcccaca ttgccaatgg tctatatcat |
| 77581 | tttcacctgg atgcctccca gacacctcag tcacatcaag ttgaaataga actaattaga |
| 77641 | cagaagtagc atgtgtttca gtggaactga catttttcta gggtccctaa aacacaggtg |
| 77701 | ttgtttcaaa ctcccccctc caacatccct tatattctct ctgtcaccca ggctccttat |
| 77761 | atcatctctc aacatgcttc ctgggtgact actgactctc cacttctggt gacaccccac |
| 77821 | agagcaagct ttcaattcct gtcatttaaa cactttagtg cactcataga atctcatcct |
| 77881 | tctgagattt ctcttccacc agtctatttt gcagaccacc aggctacgcc cttccaaaag |
| 77941 | ctcctttgag atggagtttc actcttgttg cccaggctgg agtgcaatgg catgatctcg |
| 78001 | gctcattgca actgccacct ctcaggttca aatggttctc gtgcctcagc ctccccagta |
| 78061 | gctggggtta aagttgcctg ccaccatacc cagctaaatt ttgtattagg ggttttacca |
| 78121 | tgttggccag gctggtctcg aactcctgac ctcaggtgat ctgccaccta ggcctcccaa |
| 78181 | agtgctggga ttacaggtgt gagaaaccac gcctagtccc ccaaagctcc atttttatcc |
| 78241 | tgtcttcatt ctctcttcaa gaacttgccc acagctgcca gagttccttc taagagttta |
| 78301 | agactctctg ttcatgtggt aagaatcttc ttaaggagct tgctttaaag gtaaaaccag |
| 78361 | atttacacat cagaatgggg agaggtgggt gggagctaag atggaatctg taattttcac |
| 78421 | aagtgccctg gatgattcta ctgcagatgg tctcctattc aactaaatag cactcatctg |
| 78481 | acccactcct tttagacccc acttctgtta agccagtgtt atcgctattt tccactttct |
| 78541 | ttcaagttct aatcatcctt aaaactccaa cagcttcagc tgaaatcagt accacactct |
| 78601 | catccttgtt tcccacccaa aaactattgt actaattgac cagacatgta ttgatgccct |
| 78661 | actatgagcc tggctccatg ataggcactt gggatagaga aatgtaccat aatcctgacc |
| 78721 | ttaataaatt tagagtctaa tggaagagat aaaaaaaatc aatgattcca atgagtgtga |
| 78781 | taaggtaagg ctagtcagaa aagtcaggtg cagaggaggg gcaactaatt aaaccagggg |
| 78841 | ttgggggaga cactgccaac tctgtgttga gtgaagttta agcagaggtt tttctttaag |
| 78901 | taaaaaatca gtttttttaa ataaaaagtt ttttaaaaag taagggggaa tagaagaagg |
| 78961 | aggtttgtcc ctaaggggaa cagtgtggag gaggaaagga ggtgaaagtg tatgctattc |
| 79021 | tgtgtactgg caaattcttt ggtgggtgat atggtttggc tctgtgtccc cacccaaatc |
| 79081 | tcaccttgaa ttgtaataat ccccacatgt catgggaggg acccagtgga aagtaattga |
| 79141 | atcatgaggg caggtttttc ctgtgctgtt ctcatgatag tgaataagtc tcaggagatc |
| 79201 | tgatgatttt ataaagggcg gttcccttgc acatgctctc ttgcctgcca ccatgtaaga |
| 79261 | catgcttttg ctcctccttt gccttccacc atgattttga ggcctcccca gctatgtgga |
| 79321 | actgtgagtc cattaaacct ctttccttta taaattaccc agtctcagct atgtctttat |
| 79381 | taggagcatg agaatagact aatacagtaa attggtactg gtagagtggg gtgctgcagt |
| 79441 | atctgaaaat gtggaagcaa ctttggaact gggtaacagg cagaggttga aacagtttgg |
| 79501 | agggctcaga agacaggaag atgtggtaaa gtttggaact tcctagagac ttgttgaatg |
| 79561 | gctttgacca aaatgctgat agtgatatgg gcaataaagt ccaggctgag gtggtctcag |
| 79621 | atagagatga ggaacttgtt gggaattgga gcaaaggaga ctcttgctat gttttagcaa |
| 79681 | agagcctggc agcattttgc ccctgcccta gagatctgtg gaaatttgaa cttgagagag |
| 79741 | atgattcagg gcacctggca gaagacactt ctaagcagca aagcattcaa gatgtcactt |
| 79801 | gagtgctgtt aaaagcattc agttttatgt attcacaaag atatggttcg gaattggaac |
| 79861 | ttatgctcaa aagggaagaa gagcataaaa gttcagaaaa ttggcagcct gatgatgtga |
| 79921 | tagaaaagaa aaacccattt tctgaggaga aattcaagcc tgctgcagac atttgcataa |
| 79981 | gtaagatgga gccaaatgtt aatcaccaag acaaagggga aaatgtctgc agggcatgtc |
| 80041 | aaggaccttt gtggcaaccc ctcccatcac aggctcaggc ctaggaggaa aaagtggttt |
| 80101 | tgtgggccca gcccggggac ccctgctcta tgcagtctag ggacttgttg ccctgcatgc |
| 80161 | cagttgctcc agccatggct gaaaggggcc aaggtacagc tcaggccatt ggttcagaga |
| 80221 | tgcaagcctc aagccttggt ggcttacacg tagtgttggg cctgtgggtt cacagaagtc |
| 80281 | aagaattgag gtggatgtac agaaatgcct ggatgtccag gcagaagttt gctgcagggg |
| 80341 | tggggccctc atggagaatc tctgctaggg gagtatggaa gggaaatgtg gggctggagt |
| 80401 | ccccacacag agtgcccact gggacactgc ttggtggagc tgtgagaaga gggacaccat |
| 80461 | cctccagact ccagaatggt ggatccacca acagtttgca ccatgtgctt ggaaaagctg |
| 80521 | cagataccca acaccagccc atcaaagcaa ccacaagggg ggctgtaccc tgcaaagcca |
| 80581 | caggggcaga gttgcccaag gctgtggtgg gaacccacct cttgaatctc tatgagacat |
| 80641 | ggagtcaaag gagattattt tggaacttta aggtttgact gctttattgg atttcagact |
| 80701 | cgcataaggc tgatagcccc tttgttttgg acaatttctc ccattttgaa caggtatttt |
| 80761 | tacccaatgt ctacacccac attttatctg ggaaatagct aacttgcttt tgattttaca |
| 80821 | ggcacatagg tggaagggac ttgccttgtt tcagatgaga ctttgaactg tggacttttg |
| 80881 | agttaatgct gaaataagtc tttgggggac tgttgggaag gcatgatttg tcttgaaatg |
| 80941 | tgaggacatg agatttggga ggggccgggg tgaaatgata tggtttgtct ctgtgtcccc |
| 81001 | agcttcatca ggatcctccc acacatcccc attctaagtt gcagggtccc attcttttcc |
| 81061 | aatcaatgcc ctcattttaa cagtagacac ctggtgaggc tgtgcatgca cctttcattg |
| 81121 | caggtcagcc gctcccatga taagagcttg tatctgattt tccacaattt cagctctttc |
| 81181 | tctacaagag ataagactct cactctggac aatcttagaa gatttgaggc tcagtgtatg |
| 81241 | cttctggagc tgggagttag aatccctaag ttcatggttt tcttctatca gtttgtccag |
| 81301 | tgaacttagg agcaaccaac caacttcgtt atattccttg gtcctctata tatggtcaaa |
| 81361 | ggtattatgt atagagtcat taaactcctt gcctctcatg agtggtgaat caggagtact |
| 81421 | gaatgcattt cttttgcata agtgtttgaa cagttcgtgc caaggactat cagtgttctc |
| 81481 | cacacaatta gaagtagagt ccttagcatt ttggggtcta atcatattaa gcaaccaact |
| 81541 | ccagaaaccc aaaaaccaac taaagaaatc tatccttctg taatcccagc actttgggag |
| 81601 | gccaaggcag gcggatcacg aggtcaggag actgagacca cagtgaaacc ccatctctgc |
| 81661 | taaaaataca aaaaattagc caggcatggt ggcgggcacc tgtagtccca gctactcggg |
| 81721 | aggctgaggc aggagaatgg catgaatcca ggaggcggag cttgcagtga gctgagattg |
| 81781 | cgccactgca ctccagcccg ggtgacagag tgagactcca gctcaaaaac aaaaaaaaag |
| 81841 | aaaaaaaaga aatccatcct taaaattctg ttcctctaga accattccca gtaccaaaat |
| 81901 | ctgattaaaa aaaaaaaaca ggcagaggaa ggtggaagga ctagatcttt ctccagtgct |
| 81961 | ggatgcttcc tgccctggaa catcagactc caagttcttc agcttttgga ctcttggact |
| 82021 | tacaacagta atttgccagg ggctctttgg cacttggcca cagactgcag gctgcactat |
| 82081 | cagcttccct atttttgagg ttttgggact cagactggct tcctgactcc tcagcttgca |
| 82141 | gatgcctatt gtgggacttg gtatcttgtg attgtgtgag tcaactctcc taataaactc |
| 82201 | cccttcatat attcatctat cctattagtt ctgtaccttt agagaacact gactaatgcc |
| 82261 | gtggggttaa gccaatcgtc tagtaggttc ataaaatgct aaaagcagat ataaacttaa |
| 82321 | agacgatcca gctcatctct tcattttaca gatgtaaaaa cagaagtaga aatagaattg |
| 82381 | gaaccccagt ttcttgaaac ccagtgtttg cataatacca tgctaatttc attctaattt |
| 82441 | gtgttttatt taataatcag gaaacagttc aaatagaggc ccagggctct gaaacattgc |
| 82501 | ccaaggtcta tggcttttgc aaagcaagta ctgtttctgc tactttacct agttgctctt |
| 82561 | ggcctatgtt tggggtgcat ctaaagaact gtttgctgat tattaaataa aacacaaatt |
| 82621 | agaatgaaat tagcatggtg ttataaaaac acagggtttc aagaaactgg ggttccagtt |
| 82681 | ctatttctac ctctaggata cagggtaagt cacttccttt ctcagattct gtttctttgt |
| 82741 | atgtcaaagg gctggattag ataaacttga cttcccctct tggccctcat gtatcattct |
| 82801 | ataaatatgt tatcatttct aatagactgt ttgatgtaat cttttgtcta atggcccctg |
| 82861 | cttttcacaa taaatgaaac aaaggtcaca agacttttat tcatttgcaa ccctgattaa |
| 82921 | ctaacagtta atgtgtaact ggagtgccac atagaaacag aaaggagaag gggaatggat |
| 82981 | tgggttggag aaggtgaagt ctgacctatc ctctacaaga ggtacagggt ttatccagga |
| 83041 | agacaggagt gccatgagga gtaaactcca gcaggggctg gagtcatggc cacacacagg |
| 83101 | gccttacagg atccaggctg cccagagcag agtttggatg ggggctggga ggctggggga |
| 83161 | gctagctgag gagatggttg tcatgccagg ccacgagtgt ggatgagtgc tggccagggg |
| 83221 | aggtatagga gattgatggg gctgtggcaa gcagggcaga tacccttcca ggaacctgtt |
| 83281 | tcattaaaca cagaatacag tcctgagggc tcggtctcaa tacagtcctg agggctcggt |
| 83341 | ctcaggaaaa catgttctta agttttacat ccttcttctg ttttgattag gtgtttcctg |
| 83401 | attataaaat aaattcctag aactgttaat ggtaacaaca caaagcacta tagacccata |
| 83461 | cgaggatcat attgaaacca atgacattta agaataaaag atctgatgaa tatgaacact |
| 83521 | tccctggttc ctaggaggat aacagttgag ttttgctcag gtaatctgcc ttctcttcct |
| 83581 | tcttctctgc tttRtatctc agtttctata ttttgtctta cattaatagg atttgttctc |
| 83641 | attaccctga tgattttatg gtaaactacc tcaaattctt tttggaagaa gatgggatat |
| 83701 | aaattatttt taaagtttat ctgaatagat gttcttcaat atcacacaat aaataatgac |
| 83761 | aatgtaactt tgtgcatata gcactttaag agattgatca taaacacaat cctattaatc |
| 83821 | ttaattcaag ttaataatgc ttgcatttgt atggcatttt aggtattata aagttttttt |
| 83881 | taattgtggt aagatatgca taaaatttac tatttaaacc tttttgaagt gtatagttca |
| 83941 | gtagtgttaa gtacattcac aatgttgtgt aaccatcacc actagccatt tccagacttt |
| 84001 | ttcatcatcc caaactgaat ctctgtacct attaaacatg acctcattct ccacctcccc |
| 84061 | acagctcctg ggaacctcta ttctactttc tgtatgaatt ttcctattct aggtgtctca |
| 84121 | tataagtgga atcatacaat attcatcctt tggtgtctgg cttatttcac gtagcgtaat |
| 84181 | gctttcaagg ttcattcatg ttgtagcatt tatcagaatt tgattcattt ttaaggctga |
| 84241 | atatcttcca ttttatgtgt ctaccacatt ttgcttatcc attcttctgt tgatgaacac |
| 84301 | ctgggttgtt ttcacctttt ggctattgtg aataatgctg ctatgaacac tgatgtgcaa |
| 84361 | gtacctgtct gagtctctgc tttcaatttt gggcatatac ctagaagtgg gattgctgga |
| 84421 | tcataggatc attctatttt taactttttt gaggaattgc cataccaccc gctacagcag |
| 84481 | ccgcatcttt tatattgcca acagtgcaca aaggctctga tttctccact ttctggtcaa |
| 84541 | catttagatt atcattcttt ttttaaaaaa acgtaataSc taacccaatg ggcatgaagt |
| 84601 | aaggttgttt ttgttttttg tttttaatgt gtgcgttatc ttacttgatc cattaaatcc |
| 84661 | ctaacaagaa ctcctctagg gcagatgtcc tgtcttattc atccttggcc ccagtgtctt |
| 84721 | gcaagcaagt gaattctcaa taagtgttaa ttgaatggat ggttgataga tttataggat |
| 84781 | gcatgccaat tctgtggacg agagtaggta ctaattgtta ttttcatttc acagaagagc |
| 84841 | aagtcagggc tccgagcact aagtgacttg gctgaggtca aactgcctgc aagttttatc |
| 84901 | taatagtgac agaggaacca atgtgtcgag catgaatgtc agtccattga aacagtgccc |
| 84961 | acttttctga ctctgctcct taagagacag ggcctgtaca gcaaggacac agagaagcag |
| 85021 | gttacagaaa aagggctggc tcatccgtgt atgcctggca tttgaggaat gtggctgaaa |
| 85081 | tctcaacact ctggttcaga agcacatctg caatcaaata taacaagaca tggtatgaga |
| 85141 | gatgtctggc ataccaagga gattcctaga atacagcgga taggaaaacc tcattacttc |
| 85201 | aattcccaag aaagagtact actggtagta atcccaacag gagtatccaa gtaactcggt |
| 85261 | aatcttcagt aaagaaaaga aattgtgaaa caattatagt cagtcctcca tctctgtagg |
| 85321 | ttccacatct gtggattcaa ctcacctcat attgaaaata ttaataaata aataaataat |
| 85381 | aacaatataa caattaaaat aatgcaaatt ttaaaagcat aacaactatt tacatagtat |
| 85441 | ttatattgta ttaggtatta taagtaatcc agagattatt taaaacatat gggagagtgt |
| 85501 | gcataggtta tatgcaaata ctacaccatt ttacataaga gacttgggca tctgtggact |
| 85561 | ttgttatctg caagggtcct ggaaccaatt ccccatggat actgagggtc aactgtacca |
| 85621 | agYtgaaaac aaaacaaaag gaaatctatt gcaaaaagga aattcccaaa gataaaaagc |
| 85681 | atgtctatga aatagtcaaa gagccgaaaa tgttgggaag acccacactg ctttccctgc |
| 85741 | cctgtgcccc tttttgcaga tctatttgtg tctttatcta ccagcagata ttctattatt |
| 85801 | ctaatagatt cctgtttttc ccaagagggt ttacttattt attaaatatg atgcaaacat |
| 85861 | ctctctcaga gatgttgctc tctcttccta ggattcctta gccctaaatc tgcagagcca |
| 85921 | attaggtatt aatattgggc tttacaactt tggaccatct gaccaccaag actgattcat |
| 85981 | taaatgtata tggtttcaga tcacattaat ttatcataga ctttagaatt ggctttttag |
| 86041 | aagtactgct ataaggaaaa tctcagcata gcaagtttta tctaatagtc tacttgtatt |
| 86101 | aaagagtact caatgtaaac cctaggaaga cttttaactg ccttttggaa attggttgag |
| 86161 | tgggatttga accgtgtact ctctgtaaag caggaaatta tcacttagta attactaagt |
| 86221 | atttaaaaat gggaaataga aaattacatt tcagacctgg tgcagtggct catacctgta |
| 86281 | atcctaacac tttggcaggt ggaggcaggt ggatcgcttg agcccaggag ttcaagacca |
| 86341 | gcctgggcaa catagtgagc aactccattt ctactaaaaa taaaaaaaat tatttgggcg |
| 86401 | tgatggggtg cacctgtagt ccccactact caggaggctg agacaggaag attgcttgag |
| 86461 | cctagaaggt caagcctgag tgagcagtga ccggccattg tattccagcc tgagcaacac |
| 86521 | agcgagaccc tgtctcaaaa aaaatgtata tttttaaaaa aagaaaatta cattttagtt |
| 86581 | gttccataaa tatcagtaca accaaaccta agtgcaaaat tcccaacaca aatgatcgcc |
| 86641 | tctgggccag ccatgtagcc cccatcttgc tgtcaataat catttccagg ggctgtaatt |
| 86701 | gtcttccttc cccctgtacc accccatcac aggacacaaa ttgttttgct tagtttagat |
| 86761 | agctgtgact aaactaagtg ccaattgtct tttaaaatat gtgttaatca gcactcaaga |
| 86821 | ttgttcctac aaaaatgtca ctccctcact caatttgcat gtgccctcct gagtaggaga |
| 86881 | gaaagagctg agttagaggc agccctctgg gacctgcaca gaaccctgta tgcttccggg |
| 86941 | agtgtggagt gtgtgtctga tctgctgctg ggaaaaggag aagaaatgct gagacactca |
| 87001 | ctgccagggg ctggtatcag gccattttca caggggctgt tggagggttg acagcacagc |
| 87061 | tctactggac cagggagggt cccagccagc agggcctgcc cctccgaaac tgtccctgtc |
| 87121 | cctgtccctg agaggcccca ctgagtgtca gatggcacat aagagatttc cttatgcgtt |
| 87181 | ggtgtgaagg agatgatcag ttccaggagg cccctccccc atgcagaaga gaagaaaatg |
| 87241 | gaagaaaccg ggttctcaga gtggcctgcg ggtgagtgcc gccttgtgct gtcagttccc |
| 87301 | ttcaccttcc agttctgggt gtacctagtg tggtttcatc aaactgctga ggccctggaa |
| 87361 | ttgagggagg atgctgaagg gtgccaggcc atagaagcag tagcaggagc tgcagcaaag |
| 87421 | agctagtgtg tctccagcgt cagcttttgg gccttggtgg catcaaagga gacgtacaca |
| 87481 | gggccactgt actacggatg ggattcttgg aacaaaagtt tgaataattg atgagtgtgg |
| 87541 | aagtgctatg caaaaattca attcaacctg cagttaccag gcattcattc tgtgacaggc |
| 87601 | attattgtga gggtggcaag gagacgggga gtctctgaga gactcagaga agagcagggc |
| 87661 | atggtgtcca cccacaagag atctgaagac tttcaaagta gttgggaaaa cagacataca |
| 87721 | acttttttcc cctccccaca gctagttgag gcaaaagaca tataacttat acagtgacaa |
| 87781 | aatactactg actgttactg acacataaca gcctgggctg ggtcactggt gagaaaaagg |
| 87841 | aggattcttg cttttggggc attcaagtcc tgtccattaa ccatttccca atcccatatt |
| 87901 | tatacatccc attttagaca tattaagctg aagattgatg tgacataccc aaagacactt |
| 87961 | aaggttgaag ctgagaatct gggctagaaa tataaatcag gatgggctgg gagcggtggt |
| 88021 | tcacgcctgt agtcccagca ctttgggagg ccgaggcggg cggatcactt gaagtcagga |
| 88081 | gttcaagacc tgcctggcca acatggtgaa accccatctc caccaaaaat ataaaaaatt |
| 88141 | agccaggtgt ggtggtgcat gactgtaatc ccagctactc gggaggctga ggcaggagag |
| 88201 | tcacttgaac ccgggaggca gaggttacag tgaaaggaga tcgtgccact gcactccagc |
| 88261 | ctgggtgaca gagcgatact tcgtttcaaa aaaagaaaaa aaaaaaaaag aaagaaatgt |
| 88321 | aagtcaagat taaagacaat gggtgagatc agcaaggagc atgtgtgcgg agaagagaac |
| 88381 | accaaggaag gctgcgtgtg gtgggaggtg gccggggggc agagaaagag gcggcggagc |
| 88441 | caaggagata gggcatcgtc tgaatggtga tgctgtatca acagatgtga aattcccaga |
| 88501 | ggtgtgaaac acagcagatc ctttacagca cgatgacagg acacagcatg agcctacctc |
| 88561 | tgccaggtga gaggagcttt ctgcaacctg tgatgggctc aaggatgctg accattcatc |
| 88621 | cgcccattgg gatagcccag gctctgagct cagcactcca cccgtcactg cgattgcact |
| 88681 | aatcctcacc cacccttgca ggcaggtatt actgtgggca cagagaagtt tgctaacttg |
| 88741 | ccagagatcc tgtgtaggaa gccaggtcag gaaacaacca gagtctctct aacagcccag |
| 88801 | gccttgaatg aacaccggcc tgcttcagaa tacatggccc gtgatgtgtt tgaattcaca |
| 88861 | gattcactgg acaggttccc tatagggcct gtgaggaaat ctggtctaac agaatccaga |
| 88921 | aagacaaatt tcgctaaaca ggtcaagcct gatactgctg ctacacttca gctgtgttaa |
| 88981 | gccactcggt tatcagacct gcttctcctt tcatgatttt agtaacacag gcctcttcct |
| 89041 | tgggcccctg ttgctcccac catctcccag gttctcttgt actcagggtt tagtctcccc |
| 89101 | tccccactac taatgatgca tgtgccttac tctctgatca tctctcctaa atctgctact |
| 89161 | cctctgctct ttctgtgcct tatctatccc tgccgagtct aacatgcaga ttttggtcat |
| 89221 | tccaaagtca tgtatagatc taatcacagg gctctctgct taccagctgc tacttggata |
| 89281 | aggaaagcat gccaacacgg tcctccttct tcatgctggc caagtcagca tcattattat |
| 89341 | tacctaagtt tatttctaac acatctcaat atcttcatgc actccctctt gataaaagta |
| 89401 | actgagcata gcaccatcaa taccatcaaa tctgtcattc tcttcccctc tctctgggtg |
| 89461 | ggacaggaag ccaggctgct ctaggaaatc ttccctaaca agcaaagggg acttgcctgt |
| 89521 | cctctcgcat gtgtgatctg agcttgtgtg gatcccagag tgggcattct ggactacttg |
| 89581 | accttgccta tctctccttc acaccctctc atctctccct cctaccacca aaaaacttgc |
| 89641 | atcgtatttc caatctccag acatactttt gaaaccattt atctatctgg tgtgtttatc |
| 89701 | tgtatctagt atgatgtgaa tatgtgattt gtatgtgtgt ctaccactgt tttggtcagt |
| 89761 | ttgtatttct cgtgggtaca gttctatgtg ctcatgtatg tggaacatat gtatactgac |
| 89821 | acatggacct agctccaaat gatctgaaag gaatataatt gtaattgaat atttgcacag |
| 89881 | atatacaaca tacacatgtg atggctgggg gaaatgcatg tgggatttca gtcagcattt |
| 89941 | tattagagaa ggtatgtcat tagtgctgta ttaacaatga atcagcttat ttgtgggtca |
| 90001 | ctgtcaatga ctcctttgca aatcacacat gtaaatattt ctgtctgtgg tctgatgaac |
| 90061 | atgcagtgcc acagtctggg agatgctgag ccatgccctg tgtaggcagc atatgaaaag |
| 90121 | aactgcatga tttaaaagat gctgaccagc ttaaggaaag caatttaaaa acttcctaaa |
| 90181 | aatctagttt gaatgaacaa cagttttcat tttgtgtgtg tgttttttct tttagagatg |
| 90241 | gggtcttgct atgttggcca cgctggtctc taactcctgg gatcaagcaa tcctcctgcg |
| 90301 | gctcagcctc ccaaagtgat gggattacag gtgtgagcca ccgtgtctgc tggccccact |
| 90361 | gttttaaacc ctgattcgac aatcatacat ttaactcatt acctgtcttg ttccttttac |
| 90421 | gacaaactca aagcttttat tttactaaag tatttgggtt aatcttttgc ttttctgtca |
| 90481 | tgttctgaaa tatgtacaat aacagaatcg ctcaaaatat tatctccgtt aaatgttttg |
| 90541 | tggctttagg gagaggtcta acaacatgcg ggaaacaaga aatcaagcgc atccaggatt |
| 90601 | catttataat ctctctcgtt gagtagaagt ccgcatctct cgatattgtc tggttacctg |
| 90661 | catgaagtat tctaaaggag gaaaatagct caaagaggac attcatgtgc actctggctt |
| 90721 | ccagttggcc atttgagtaa gtgatcgcaa ttaactgacg agcggcaggg aaacacttcc |
| 90781 | tggaattctc atctacagac aagaacaaac tggggcgggg cccatcacct tcacctacgc |
| 90841 | gccgggaggg tggcggctgg cgggcggggc cgggctcggg ccgtgacgcc gagagtgcgg |
| 90901 | ggcgcgcggc tgggagcctc gcgcccccgc ccgggcccgc ccccatcccg cccgcataca |
| 90961 | gcccgcatcc cgccggggaa gcgagcccag tccagcgctg cccgtccagt cctcgcccaa |
| 91021 | gatttaaagc ccgcaagttt tgttcttgag accagcgact ttagctccga tgcgggaagg |
| 91081 | aaagccgacc tccgatttgg acatttaaag agctgggctt gaacttcgtg agtttcgctc |
| 91141 | taaactgccc ttgaaatgaa gctggacttg gaggtaaagt cactgggaag ctggcctggg |
| 91201 | gcggggtttc cccctcttct cgtattttag aaacggacag cggcagtgca gccctagttt |
| 91261 | gctgtaagtt tccttacttt gttactgagg cccccagagc tccacgcata agtggtgtga |
| 91321 | ccagaaacct tttaacaaga cccgcctgag cctgcgttag agctcccgct cggaaagtaa |
| 91381 | aagaccatcc taatccgcgg cgctgcggaa ccggtgtccc gtgtgggagg aaccgcggcg |
| 91441 | ttccctgggc gtagggcccg cgaggccagc acagtccgcc tcttggcgga gcgccctggg |
| 91501 | ccggtggttc cgcgcggagt tagtctgtgg tcagttacgt ggtgaaaaca cggctgtgcc |
| 91561 | gcggccgcat ctttccgcgg ccgaggcctc tctgggtggg agtgttggct tcctttccgg |
| 91621 | atcgctaaat ggggaaagtt ctggccgctc ggcgggatac gtctccaggc cacggatggt |
| 91681 | tcgttctccg tgccgcggcc ccgagctggg ctccctgggt ctccagcgcg ggctcccggc |
| 91741 | attgggggct gcgggccggc ccctccgccc cgcccccgcc ccgccgcgcc tcctcggccg |
| 91801 | agcggctcgc ggtctccggc gcgggaggct ccgagtctgc ccactccggg ccgagcgagg |
| 91861 | tctctggagg agaagagtgg cgaggaggtg agggcacgcc ggccctcgcc cggcgggtgg |
| 91921 | cgccaggact tcaggtggga acgcgcgctt gggccggggg cgcgtggctg gcgtggacac |
| 91981 | cggatcgggg cccgccgccc tggcccggac cgcgcacggc ccagcgccgg gaagtcggga |
| 92041 | agccggggag gcctctccca ccgcgggccc cggcagcccg ccctctgaaa gcgcggcgga |
| 92101 | gaaggaggct cgtcccctcc ccggaacgcc tttgttccct ccggcctgcc cgcgcgggtg |
| 92161 | gccagcggct gggacccagg ccgggccgcc gcccaggtgc ggcaggtagg ctcgggggcc |
| 92221 | gggcagctcc ggttggggcg gcttcccggg gcctgcgggt ccccgtccct gaggagctcc |
| 92281 | ggctcctcgg tggcgggaca ggcccgtgcg cgggagccgc gaggcgaacg ccgcgcccac |
| 92341 | caattcggtt gccggccggg ggccccaggc ttgcggccac ccgcctccgg ctggagggct |
| 92401 | gaattcgagt cgaaagcccg tgtcgggctg gaaagaagaa accgccaacc tgagaacgct |
| 92461 | ttcggcgagt tactggcggg ggaaatgggg acagggaagt gggcaggcgg ggagactgca |
| 92521 | gccgcagatc tccctggcgg ggaggtcgtg gccactcttt cctttgactc tgcctcattt |
| 92581 | cattttgaat cctgatgtga cagaggcaat tgcttgcttg gataccatat aggtaaaagt |
| 92641 | aacagttttc aactcgactc ttgactacac cctgtacatt cttggccggt gttggttttc |
| 92701 | ttaggttatg gatcatgtta aaggtacacc gatgtggtaa ccgcacagtg gccgatggtg |
| 92761 | gcctgaggct catatttatg gtaattatct gatgaaagta catccatcag aattggattt |
| 92821 | gtgcgtctgt gcctttattt tgggaaacct tgcctgttcc ctgtggggga tgggagagga |
| 92881 | atgtgaaaag ccgaagttga cccagaaaag gatgattgaa ggtagttgta ttaagtggtg |
| 92941 | gcaccgaaat gattccaccc tgaactttct gaaagggtga ttagtgatca gcagcagacg |
| 93001 | ttataacttt ctcaaaataa atttatggta gattttctat ctggtcacaa gtgaggaggt |
| 93061 | gaaagctgct ttttggggcc aactctttgc ttttaaagca agctaaacgc aataccagaa |
| 93121 | aggtttccat tctgtactta acctgcttgt ccttctcact cttccttatc ctcccgcaca |
| 93181 | cgctctgagc attgactgag cactctgagg aggaggctga ctcaggctga cccttcccgg |
| 93241 | ccctgcaagg ctctaaggta gaaccagtgt tatcacagga aaggcccaag ccaaagctca |
| 93301 | gagcagctgt tcctgagaaa gtgggagatg aggatgagta ggaggtagag atgcttataa |
| 93361 | ctgctctgcc cagagaaaac tcagaaggtg cagaagagtt ttcaaaataa agtgcaggcc |
| 93421 | ccatcaagta aaaaatgaaa cattgtattt cattaaaatg gtgatcagtt ttctcttttc |
| 93481 | ataaaagaca ttcaaatggt gagattgtaa aaaaaaaaaa aaagaaaaag aaaactttct |
| 93541 | aattctcaaa aatagaacag cttgaataat aactcatggg ttttgaaatg ggtcatcttt |
| 93601 | taaaatgggt cactttggct agaatcattg cacttggtga ctttcacatt gtaaagggtc |
| 93661 | cagttctttc tgaggcccaa ttgcactaga atctgcattt cagaacacag gaggtatcag |
| 93721 | gaagaaactg ggagaaattc agagggaaat tgtctcttct cagagtaaca aaatgtcccc |
| 93781 | tattcagggg gaattttata tatgctgaag taaatagatc tcagctttga atttttataa |
| 93841 | tacattatgc cttttctgaa atatttccat agccattatt ttaatgcatt gttagaataa |
| 93901 | ccttgtaagg agaggaattc attgtatctg tgttccagaa actgaggcac agtaaattac |
| 93961 | aagagctctg ccctttgata tcgaatctct gtgagcgaag atgtaaccag aacagtgtaa |
| 94021 | atctagaatt actttcctta cggtatgcaa tatctaatta ttatgtatta gtttacatat |
| 94081 | atatagggga tgtaagaatc tttatattta aaggataaga aacttgatca ggtgagatac |
| 94141 | aagatttgaa taaaaaagcg attttttaga aacattttaa ttctacaaat tcagttgctg |
| 94201 | gctttgatat gtaacttatt cagatttctc ttccatagaa ggctttaaca ctagaagcca |
| 94261 | gttctctaca aaagagaatg ttctaccaag tgtgtaggca acaaagccca aaagttcaag |
| 94321 | ttcaaaatac tgttctacag cctaagatcc acctaatatt tcagatttga ctcttttgct |
| 94381 | cctttcctat aacttcctat aactttttca tagtgctcta agcaaagtaa gttcaaggat |
| 94441 | ggactcacct caagtttcct tctcttactt tagaggacca tataatctta tacctgtgtc |
| 94501 | atcctaattg aaatgtattt taagtgcttg tgggaacaag aataacggaa gaccgttatt |
| 94561 | ccattaatgg aataatgggg aacatgagaa ggaccatatt cagtcaatta gggatgatac |
| 94621 | ttctgcctca acagtatttg cgatgtgtta aatggccctt agccatcaga tcagtatttt |
| 94681 | taaaaactcc tacctaatta atgttttttg agaagaagca tattatgagt atagctcagt |
| 94741 | attctaaaaa gaaaaaaacc tgaaaaaaaa atcagccatc cattaactaa cccatcctgg |
| 94801 | taaagctaca caaacagatt ctagagaaag gaaatttgca cagatggaac atctaatctg |
| 94861 | gactgacatt gtcaacaagt tatctcaaat gatcctgaga aaacactggt acatggttta |
| 94921 | gagacaagtc gagctcatgt gactagtaaa tggagaggca cagtatacat ctacttttgt |
| 94981 | tggcatttaa acactctcgg cttttttact ctctttcacc accatcaggt ccagattcca |
| 95041 | gcttgcactg gaatatttat tgaccctttg atagaagagc tacacctgaa acatctctga |
| 95101 | agtcttttta gcatcctatc ctgcccatgg cctactacac agtatgtgat aagttaatgt |
| 95161 | ttgttgaatt agttaaatat acgtattaac ctactttcag ggctgcccgc tgtaatgcct |
| 95221 | agaatagggc aggcacttaa taaatggtag ttaacttaga ctaaatgttg atccaccaag |
| 95281 | acagaaacat gttacatata cctcttgttt tacaccatct tctacttttt gtacctctgt |
| 95341 | gttataaacc ctctatcaag tgttgcttct aaagacaatg tccagaatgg gacgtaggaa |
| 95401 | ctgccactgg gcaacaggca gacactgggt tatatcgtca tcctgtttct gaatcctggt |
| 95461 | ttttcttatt attattcaac cacagctaaa ctttctgatc tccttttttc cgttttttcc |
| 95521 | ctttacccta acttctagcc aaactaagtg atttgacatt tccccactca agccacaact |
| 95581 | ttttctctta tgtgcttaaa aaaaattcct gcgtagtctc tcttggaatg tcttattcct |
| 95641 | tgctccctcc acttcaccct tttttttttt tttttttttt ttttttgaga cggagtctcg |
| 95701 | ctctgttgcc caggctggag tgcaatggtg ccatctcggc tcaccgcaac ctccgcctcc |
| 95761 | tgggttcaag cgattctcct gcttcagcct cccgagtagc tgggattaca ggcatgctcc |
| 95821 | accacaccca gctaattttt gtgtttttag tagagatggg gtttcaccat gttagccagg |
| 95881 | atggtcttga tctcccgacc tcctgatccg cccgcctcgg cctcccaaag tgctgggatt |
| 95941 | acaggcatga gccaccgcac ccggcccact tcacccttta agccagttga aatgctcctt |
| 96001 | gccctactcc tcccttctcc ttttctttct cccatccaag tactaaccag gcccaaccct |
| 96061 | gcttagcttc tgagatcagc caggatcagg tgcattcagc ggggtatggc tgtagacttc |
| 96121 | tctttctgag ctgtgtcttc ctcctttcct ccccaaaccc acatttgaat tctgttctag |
| 96181 | ctcctgctca tttgccacct ctcccacaaa gccttcccca gtttccatct tctccctgct |
| 96241 | tccctcatct cagaggaaag ctcttcattc acagagcacc tggtttgggg tccatagttt |
| 96301 | gttgtccctc agtatccttg gggaattagt cccaggacac cacccccccg cccctgatat |
| 96361 | ccatgcatgc tcaagtccct tatataaaat ggtacagcat ttatattata acctatgcaa |
| 96421 | tattcccatg tactcttttt ttttattttt atgtgtttag agatgggtct cactctgttg |
| 96481 | tccaggctag agtgcagagg catgtagcct tgagctcctg agtcaaatga tcctcctgcc |
| 96541 | tccgcctccc aagtagttgg gattacaagt atgagctacc acacctggcc catgtacttt |
| 96601 | aaatcatctc tagattactt ataataacta atgcaatata aatgctatgt aaacaattgt |
| 96661 | tacacggtat tgtttagtgc ataatgacaa gaaaaaaatg tgcatgttca gtacagacac |
| 96721 | gaccataaat ttttttttca aatgtttttg atctgtagtt ggttgaatct gaggatgcgg |
| 96781 | aacccatgaa cgtacacgac caactgcata tgggtttcta ataacagatt tgtggatgaa |
| 96841 | gcccacccaa cataatgcaa catttcaaat gtatcttagt cagtttttct ggggcaaagc |
| 96901 | cagcctaaca aatctcaaga atgtttgaaa aattctagct tgagtgaacg ttgttcttct |
| 96961 | agtgactcaa ataaagcagt cattgtgtct gaggagtcct agtggggtgg caggggtggg |
| 97021 | gctaggagta gtggctgttt gtagcatgtt ttatatcaca ttaaagggct gggtatgcat |
| 97081 | tttaggcaat aattcttact cttattgata ggagaacttg tattttttat tcatctacct |
| 97141 | tatgatggat aactggttta catctcttca tcaggtttac ttcttacgtg tcattttatg |
| 97201 | agctgacttt gaatatatct tcagattttc cctgccttta cacatgaaaa cggttttgat |
| 97261 | acagcttttt cattagagaa tggcagcttt tccaggccag ggtgtctgtg tctgactcct |
| 97321 | cgcctttcat tttacccaat acttgcttaa aacatcctcc ctgagtccgg tacttttttg |
| 97381 | cctcctctcc ttccttccac cccacctctt tttgtcaccc tcctgcgact ttggatctcc |
| 97441 | atccttggag tccctgaatc ttttttgtgg ttgaaagact acacccaaag gacacagact |
| 97501 | gatgaggtca cttctgccca ctgtctagta ctagtgagac caagaagaag aaggagggaa |
| 97561 | aactgaaatg gaaggtttga aaaagagtgt gttagtgaag gaagtaagaa cagtgctaac |
| 97621 | agaatgcagc aaaacagaaa tgacttcaca cttctggcag tgaagcaaat tctcatccat |
| 97681 | cttgggttgc ttctcttgac cctgcccacc tcctcccctg ccctcccctc ccgatgagaa |
| 97741 | ctgcaggaac acctgtgccc atgccaagga aaaagggcag gtgaggaaca cagggatggg |
| 97801 | gctgggactc acaagctgct ctaagctgcc acagggtctg gaatttgttg acattacatg |
| 97861 | gttaactcgg tatctcgatg cctcagtatc ttctgcaaaa tgtggaggga gatcctttct |
| 97921 | tcctcaaagg ttgttgtaag gcttaaagag ctaccacatt cgtaaggcat ttggaacagg |
| 97981 | gcctggtata tagtagacat acccatggta gtaactgttg ctagactggg taaaaggaga |
| 98041 | agtttcttaa gtaggcaaga aaaaaactga caacagttat atttaaaaga gcaagcaacc |
| 98101 | gtgtcagccc aaacaagtgt gttgcaagaa tgagcacaga gggccaagct cagaaaagcg |
| 98161 | agtgtttgac ctctctgaga cctcctttat atgtcagctt ttttatttgt ttcaaatttt |
| 98221 | gcataatcaa actggttact gggaattatt gagttcatat aacttcccta aaatctaaga |
| 98281 | ataatctata atcccatgcc accagtcccg ttaataaaga cttagggtct gtctttaccc |
| 98341 | atttgttcat ttgaaattcc ccgtctttac ttcccccata accaggaaac agatctacac |
| 98401 | ccatgtttaa cttataaaga gatataagtg agtttacata ggtggagatc ttgtacctgg |
| 98461 | attcacattg taggttttat tacccagctt tcccctctat cccaaagcca tgattgccat |
| 98521 | agtggaattt aaagtctttg gactaacact gaatcaagaa ctaactctac aaatgtattc |
| 98581 | tctgcaggag gataagaatt ccaaatggct tctaattgtt gcccatggct tgaaataaag |
| 98641 | ttccatatag ctaaagcccc agcacaaaaa cacttgaaga cagccattag gtagaatttc |
| 98701 | ttttcttttt tagaggtctt atattggaaa tgtaacagtt gcaaagatat ctaatgtttc |
| 98761 | accttaaaat gtgagttata aattcacatt tcacattgcc tccttcctcc ttgagcaaac |
| 98821 | agactaggca attagtacca aatagtaact agttatattt cttaacaccg attattgtaa |
| 98881 | gtaatttatt aagaaaaaat ctaggaagat caaattatag cttttccagt aaatctgggg |
| 98941 | ctttttattc ctgatctctc tcaacaaggc agtttggctt ctgaagattg gtcctagcta |
| 99001 | tacttaacag gaaacagatg acgagaagaa gccaggaaaa aatacttgga atatagaaca |
| 99061 | aaatgcagca taaagatggt acagaagatt ttatctttct ttcctttagt tatgtgtgga |
| 99121 | caggaactaa aaactctccc acgtgggact ttattaagta ataaaagttt aaatgtaaaa |
| 99181 | gatataaagg aatgaggata taaaggaaat ttttttaaaa actgtgactc accggaggag |
| 99241 | ccttatatag tctactttct aagcatatca cttttaaagg gatcattata ttttcatgat |
| 99301 | gcatggggat attccaagta ttaaattgat tttttaaatg aagaaaatta tcagttcagt |
| 99361 | cttgttttct atgtaggttt cactacagta ttttatttcc cccgcaagga aaaaaccaga |
| 99421 | gctagaatgg aatgattaaa tttcttagtt ttcttctaat ctgttagtct ttccattaaa |
| 99481 | ttatactgcc ttcttatata gaatttctgg gtttgttctt gttttggcaa taattgaagt |
| 99541 | aagagaggat gattttatgg tagaggtttg attccattct agattggtgt ttttcagcta |
| 99601 | tgtagaacac aatccattca tggttcagac aaccgtttta ggaggttttc atgtccagca |
| 99661 | ttaaaaaaaa aaaaaaaaga atagaattgg gcattcatat atatatatat atatgaatat |
| 99721 | ttattaataa aagtttaaaa gatataaagg aatgaggaat gcatatatat tatatatata |
| 99781 | tataccaaat taaagtatca taatactaat acaaggatta gtatcgtttt atgatacttt |
| 99841 | attatataca ctaagggtta gtcttgtttt atgatacttt attatataca ctaagggtta |
| 99901 | gtcttgtttt atgatacttt attatataca ctaagggtta gtcttgtttt atgatacttt |
| 99961 | aggttggtat acatacatat actaagaatg cagacatgtc catggaagtt ggcaatattc |
| 100021 | agcccccagc aggccccagt cagcaggaca gggattaggg gagtcaggtg aggcagggct |
| 100081 | gtataagtgc agggttggag cctgtcttgg tttaccgttt tgacattttg ttcactgtgg |
| 100141 | atttttttca gtaactttga ttttttaaaa atattgcatc aaaatattat actgactacg |
| 100201 | gagtttttgg taccccctta aattgtgcac ctaaaataag tggctccctt gtctccccct |
| 100261 | agtcccggcc cgatcagtat gttttataga gggaatggtg agtgttgggg aactagggag |
| 100321 | cccatgagga ggcggcccag aaggggagga ggagctgatg gtgtgtctga tgtctcctcc |
| 100381 | tttctctagg aagcagacag gaggtgaagt cttcagggtg ggggggacag agcctgggag |
| 100441 | ggggagattt agcatggccg ctgaagagac tgggaaaggg agaaggttcg ggacaagttc |
| 100501 | aggaacagtc acgtagcact gaaaactgct caaagctaga tagtgccatt tgtggtgacg |
| 100561 | ctgcctgcct ggtaagtccc atttccccag cagccctgga ggtgtcatag gaacaggaag |
| 100621 | atgggagggc gctggggtct ggggttgttg gggtgggagg gcaaaggtgt ccaatgattc |
| 100681 | tgcatctgaa acgggggagg tgtgtgagga agaaaggtca ggagactgct gggcagggtg |
| 100741 | gggtggcaga aggttctcga tgaatattta gaagttgcag ctgaggagtg agtgtactat |
| 100801 | agagtgagta tcctggaatt tgaggtctca gaagaaatgc agttctcagt gaagccatgt |
| 100861 | aaacaacttc ctggctgata aggtgtcttg agtgttggga gatcacatga tcaaaatggt |
| 100921 | atacacacca taaggtgtcc ttagacttga gtgtggtgta agtctactga ataaaatgca |
| 100981 | gaaatacagt tcactgttgc ttttcttcct ggaaattgcc ctccccaacc tcaaaccagc |
| 101041 | ccccaagtgg ggcagggtcc atctttagac cctcctgtag cacccagcac aaccctaact |
| 101101 | atatggtcaa tgtctctctt ccccacttag agctccaaga ccagggcctt gtttgtcctg |
| 101161 | cttacccact ctacccattt gttaacccca tcttaatttt ctgatgaatg agcccttttt |
| 101221 | tcattgactc ctcctttctc tccttctccg gagtttattt ttcctccagg cccattgctg |
| 101281 | tctctccacc tagagagctt atccacattc atgaaattag tcatcttcat gctcagagta |
| 101341 | cccacctgga tctctccagt cctgtctaac tcgtcttaga ttattacccc ctggacatct |
| 101401 | gtaccttctt gtctctccat cgcctcgaac tcaatgcgag tctaaaatca aatactcctt |
| 101461 | cactcctaac tcaggaaacc agttttccct cccttgttgg taatgcttga aattagctct |
| 101521 | tacttctctt cttcctcctt tactcgccat cccaggtcat tcatcaggtc ctgctaggtc |
| 101581 | tttgattttc ttgaaagggc cttctttatg ccttccttcc gtccccattt ccaccaccct |
| 101641 | cgactaggcc ttcctcacct catccggcat cagtgtgagc ccttgagaaa ccagcgtcct |
| 101701 | gatcttcagg cattctgcat ttcaccccat accaccatta gccagtttaa tccctcttgc |
| 101761 | tcaaacttaa taaatactga gcaagggagt tactataaca aacaaaacca gaaatttcta |
| 101821 | tgacttcctg ctgctttctg taccccccta agactcctct acctgatttt caaggttctc |
| 101881 | cataagcagg ttcccacctc tctgctttca aatcttctat ctgccataca cagttcttgt |
| 101941 | ctaaaacaaa gattgttcct actcctgcag ccacctaaat cctagtcgtc ctcaggccct |
| 102001 | cctaaaatcc tgactaatcc aacccccacc gatttctcta ctagtcccag agacttccct |
| 102061 | cttgtgcctg gggttggtgt catgcagaca cactgtacct taccaggatt tgcccattgt |
| 102121 | tgtctgtggg aactatgctt tcatgtggga actacgagtc ttgtcacaaa tagagtgtaa |
| 102181 | atgctttgag attggagact gtaaaatact tcttcatgat ctcccactgt gtctggtata |
| 102241 | agaacccgca gagtaaatac tcatatactt gtaggattaa ttgaaacaac tggagactga |
| 102301 | ggttgtttaa cttgtactta gaagtgtgga cctccccatc ttgattaaaa cttcaaaacg |
| 102361 | gatagcaggt ttcacatatt atctcagcat cacaaaagta gactcaacaa agaaatgagt |
| 102421 | tgacagcagg ggtaaggttt acaacaatca gggattattg cagggaaact tgtagccttt |
| 102481 | gggtgttcta actagttttt tcccaaaaag tttaaagagc tctcttgtga aagaatattc |
| 102541 | ttatgagtaa ctgaggggca cggctcctag actgaaaagt atttgggatc tgtcacctct |
| 102601 | tttctgatgt tcctacttct attctttatt cttctgacta cctctattag aaaagataat |
| 102661 | actaagaata cctgtccctt cttctcagtc caaatagaag caaacccagg ttgtatctga |
| 102721 | gatatctgca tattttcttc taagcaattc tttgtcctct tctctcccat gctttttcta |
| 102781 | tttcctattt tcagcaggct ctgaagtcat ttataatttt tactgcccct cggtgacatt |
| 102841 | acatggatat tcatccctga tttgcagatt aaagcaccga atcagagtga ggtgagggtt |
| 102901 | gcccaaggtt acacaataaa tctggcccca aacaggggta acccttgagt ttctagtctg |
| 102961 | ttgattggcc actgacccgt gctgcaggca cacaaaggaa gctgcaccca cagcagtctg |
| 103021 | ttgtggatgg ttgctgagct gcgcattcgg cattgggctt gctttgtttc ctgccaggcc |
| 103081 | cagcattttc ttctaccaga tcggcaggct tgtgggcttc ttcctaggtc cctcccctgc |
| 103141 | actctgaata ggaaagctgg aagctgtgct ttagagaagc tttaagacgc cgaaagaaac |
| 103201 | cagaagagtg agcgccagtt gtatgtgcgt ggtctccatc cgcaaagccg gagctgggcg |
| 103261 | caacagtgtt gacttgtaat tgatcaattt agatcgggcg caggccgggg gagggcagtg |
| 103321 | cttttgattt aggctgggaa aggcctccta gtgactatgt tcaatttgga ggaattcaga |
| 103381 | tgttcttttg ttatacaagt gaagctgtgt aatacaaatg aggagtttta cttttcctaa |
| 103441 | atcttcccct tatcattcaa gtattgagga gttttacctt tcctaaatct tccccttatc |
| 103501 | attccagtat tatcagtgag atctggttgt gatttatgta aatggtggct aaaaaattca |
| 103561 | aactactgag ggggagaatt ctcattttac agcttcacat gctgtgctga actaaataag |
| 103621 | tagcgtggga tgttggcttt gtgacaggtc ttttgtcatt tttcagaaag cattttgact |
| 103681 | tgttgatgtc aatttggaac agctgaaaaa atacaggaaa ataagataaa tacgtacatg |
| 103741 | ttgagggtgg ggacaaaatg aaggttctga accagctgcc ggcttacagt agccatataa |
| 103801 | gcaacagcag caatgcacca acctggtgag taataggcct gattcactgg agagatacta |
| 103861 | gcacctttaa tgagtcagat agatgcacaa tgggtgtggg agcagttgga cttgtgggca |
| 103921 | caaagtctag caagaagctc agacttgcaa acaactgtag gacgtgcaaa gcaagctggc |
| 103981 | attggagctt gccgggcaca gctgctcagg aataggcagc tggttttccc tttgatccct |
| 104041 | gagattccaa aggttacttt cctctttgtt cccttcccag ggtcaattag agtagaaact |
| 104101 | gcagatgctt ttcagttgag aattttccta gaattctcaa aaatgtgtat gctggcttaa |
| 104161 | aatctgccat caagaattct gttaccttgc tttaagcctc cagttccttc cagatgtatg |
| 104221 | gtggaggagg ccagagggcc cttgttttgg ggcttcagag gatggttgtt atctggatga |
| 104281 | gcactgtgga aagactgaga gagcaactga gagaaagtgg gcccctgaat gaaagtgatt |
| 104341 | tcgcaaattt taggcagatg ccaccatcag aaactgatat tttctgacgt ctttctcacc |
| 104401 | ttcctctaga gcattcagtc cagaaatgac cagcctgtcc aaagggggaa attactgata |
| 104461 | ttgatctgtt ccttagagca gtgtttcagt cttttttttt tttttgagat ggaatctcat |
| 104521 | tctgtcaccc aggctggagt gcagtggcac gatctcggct cattgcaacc tccaccttcc |
| 104581 | tgattcaagt gattctcctg cctcagcctc ccaagaagct ggaattacag gtgtgcacca |
| 104641 | ccacacccgg ctaatttttg aattttttat agagatgggg tttcaccatg ttgccaggct |
| 104701 | ggtctcaaac tcctgacctc aagtgatcct cctgcctcgg cctcccaaag cgctaggatt |
| 104761 | acaggcgtga gccaccatgg ccggccttca gcctttgtga tattaaagca cagcaacaca |
| 104821 | tttcccatta cacccctgaa cacacacaca cagaaaaccc aaaagtttca caaaatgatt |
| 104881 | cttgctctta ctactctcag tacactctgt atttaaaaaa aaaaatgctg gttgtggctt |
| 104941 | cctaagtggt gcgtgcagtt ttcaaatcaa tgcccttggc gataaagtgt gccctatact |
| 105001 | gattatctct ggacaaagtc tgaatggggc ttggctctaa tctctagtcc tcattggaca |
| 105061 | ttttacatac ctggcctttg cctccaccct gatgtggagt gatcatgggg gtgggaaata |
| 105121 | tagctggatc cgaaagctct gaagtgggga tggaggtgtc acagctgagg ctaggcccat |
| 105181 | tctgcagggc actcagtgtg tacagttggt tttctatcag gggtcaaccg gcggggggac |
| 105241 | ttgagaacag atctctgggc acaaagcagg gcctttgccc tggggcttgc tatgtggctc |
| 105301 | agcctacacg gctctctccc cgtcagtcct gtccaaagcc caggaaacta atgtaccacc |
| 105361 | cccgaggaag agagcctacc tttccatcca aggaagtgtt ttacctgtgg taagcacggg |
| 105421 | ggacagaatt cttgaggaag gagggtgctg cgtcccagtg gtggaggaaa agagaggacc |
| 105481 | tggtgtaagc agccatggca tggacctcat ccgaggtggc acctggctag ggtcctgacc |
| 105541 | tccaatcctt ccccagtaac catcactttg agtaaacagt ggctccaccc ccggcatggt |
| 105601 | tctttgcacc aacatttggg gaatgcctac caggggtcac acactgagct ggatgctgag |
| 105661 | tgtagggtgt ccacaacatc gtgcctaaaa agtctctgta tggggtataa gaaggtgctg |
| 105721 | gggcaataca gatgagatga gaagcatctt tcagggaatg ggttgatccc aattcaggct |
| 105781 | tcccagagaa ggatgtctgt agacttcata ttagcaaggg aggaaggtag ccaggccaca |
| 105841 | ggactgctgg tgtaaagacc agggcatatg aaatggcaag tgtgactgtg ctttcagcca |
| 105901 | ataatttggt attgtcaaat gatgggacca aacagctgga gaggcagatc ctaaagggtc |
| 105961 | ctgtgggcca ggctggactt catcttgtca ctaactaatg gagaggctct gaaggagtta |
| 106021 | aaagagctca gtttgtctcg tggttaaatc caagttttac aaaggtcacg ctgactgtaa |
| 106081 | agtggaaggt gggctggcca ggggatcatc tagtctgggt gagaagtgat gataacatga |
| 106141 | aggggtgaag agagatttag aagaagtgat tcacaggatt aaacatttaa ataatggaag |
| 106201 | tggagaaaat ggggggggcg gttccagatt tcaggcatag atgaaagaag tgcagttagg |
| 106261 | cacatgtaaa gagaaacagg aacagcaggt tttaggggag aagataacag aatgggtgag |
| 106321 | aaatgacact tgagtaccct agtgtgctag gtaatcatct gtctacttcc cttcatttgt |
| 106381 | catgtatatt cccatttaat ttgcataaag acttcgagtt aaacggtctt accccaattt |
| 106441 | gtcaaatttc tgcgcatgat atggtacaag aaaccgtaag tggctaaggc ggcattggtg |
| 106501 | ttcaaattgc ctgactacaa aggcagtgct tgttggctac attctgttgc ttcccagttt |
| 106561 | agaacatgtt acattgaggc gcctgctgca tttccaaata aaaaagtaca gaaagaaggt |
| 106621 | ggctgtataa atctggggct cacaaagtaa ttttgattac tgagagtttg ctttcaagga |
| 106681 | gcaaactgtg actccttgat tatgaacctt aatttaaaaa aaaagaaaaa agaagtctta |
| 106741 | ctcttattcc tgccttgtct ggggcaagcc ttaatggatt tttactgctg tgaattttct |
| 106801 | tttcattgaa gattttgcct tgatctatgt atctgctttc atcctgacca tattcaagtc |
| 106861 | agtatattca tgaatgtacc tgtttgtgaa atttgaactt aagtatacac gattatagcc |
| 106921 | gtttgggaag cttttttttt ttttttttta agagtaggag tagaaaaagg tctctgtact |
| 106981 | ctgaatggga agacagtgta aagcaatttt ttcccttttc ctgtcctcct ttaaaaaaaa |
| 107041 | taaacagccg tatgcctctg ctaagtacta actacctcat caccttttgt gcagacaggg |
| 107101 | caggttacat ttggttttaa ggaattagga atatgtttct ttccagcacc ttagtaaccc |
| 107161 | acgcgattgt gattcttttc tcttcttgac tgtgataggt ggcatggaat attcacatgg |
| 107221 | gagagccgca tgaggccgcc caccacgctt cctgaaggat gcccgtgtgg aagaattttg |
| 107281 | acgtgccagt gtcctcgttc tacagggtgt tccattcttc cgcaatctca gaaaaatggg |
| 107341 | actaaaagaa actattttgt aaaataagaa gacttccatt tttaatgacc aacatgtatt |
| 107401 | aagatggaca cctactctac gaaacacgaa gttctatggt ctcgaagaag cccgtgcctg |
| 107461 | tttaaaactg atcctaacta aaaacagact tgagtggata tgagaatgtt ggttagtggc |
| 107521 | agaagagtca aaaaatggca gttaattatt cagttatttg ctacttgttt tttagcgagc |
| 107581 | ctcatgtttt tttgggaacc aatcgataat cacattgtga gccatatgaa gtcatattct |
| 107641 | tacagatacc tcataaatag ctatgacttt gtgaatgata ccctgtctct taagcacacc |
| 107701 | tcagcggggc ctcgctacca atacttgatt aaccacaagg aaaagtgtca agctcaagac |
| 107761 | gtcctccttt tactgtttgt aaaaactgct cctgaaaact atgatcgacg ttccggaatt |
| 107821 | agaaggacgt ggggcaatga aaattatgtt cggtctcagc tgaatgccaa catcaaaact |
| 107881 | ctgtttgcct taggaactcc taatccactg gagggagaag aactacaaag aaaactggct |
| 107941 | tgggaagatc aaaggtacaa tgatataatt cagcaagact ttgttgattc tttctacaat |
| 108001 | cttactctga aattacttat gcagttcagt tgggcaaata cctattgtcc acatgccaaa |
| 108061 | tttcttatga ctgctgatga tgacatattt attcacatgc caaatctgat tgagtacctt |
| 108121 | caaagtttag aacaaattgg tgttcaagac ttttggattg gtcgtgttca tcgtggtgcc |
| 108181 | cctcccatta gagataaaag cagcaaatac tacgtgtcct atgaaatgta ccagtggcca |
| 108241 | gcttaccctg actacacagc cggagctgcc tatgtaatct ccggtgatgt agctgccaaa |
| 108301 | gtctatgagg catcacagac actaaattca agtctttaca tagacgatgt gttcatgggc |
| 108361 | ctctgtgcca ataaaatagg gatagtaccg caggaccatg tgtttttttc tggagagggt |
| 108421 | aaaactcctt atcatccctg catctatgaa aaaatgatga catctcatgg acacttagaa |
| 108481 | gatctccagg acctttggaa gaatgctaca gatcctaaag taaaaaccat ttccaaaggt |
| 108541 | ttttttggtc aaatatactg cagattaatg aagataattc tcctttgtaa aattagctat |
| 108601 | gtggacacat acccttgtag ggctgcgttt atctaatagt acttgaatgt tgtatgtttt |
| 108661 | cactgtcact gagtcaaacc tggatgaaaa aaacctttaa atgttcgtct ataccctaag |
| 108721 | taaaatgagg acgaaagaca aatattttga aagcctagtc catcagaatg tttctttgat |
| 108781 | tctagaagct gtttaatatc acttatctac ttcattgcct aagttcattt caaagaattt |
| 108841 | gtatttagaa aaggtttata ttattagtga aaacaaaact aaagggaagt tcaagttctc |
| 108901 | atgtaatgcc acatatatac ttgaggtgta gagatgttat taagaagttt tgatgttaga |
| 108961 | ataattgctt ttggaaaata ccaaatgaac gtacagtaca acatttcaag gaaatgaata |
| 109021 | tattgttaga ccaggtaagc aagtttattt ttgttaaaga gcacttggtg gaggtagtag |
| 109081 | gggcagggaa aggtcagcat aggagagaaa gttcatgaat ctggtaaaac agtctcttgt |
| 109141 | tcttaagagg agatgtagaa aaatgtgtac aatgttatta taaacagaca aatcacgtct |
| 109201 | taccacatcc atgtagctac tggtgttaga gtcattaaaa tacctttttt tgcatctttt |
| 109261 | ttcaaagttt aatgtgaact tttagaaaag tgattaatgt tgccctaata ctttatatgt |
| 109321 | ttttaatgga ttttttttta agtattagaa aatgacacat aacacgggca gctggttgct |
| 109381 | catagggtcc ttctctaggg agaaaccatt gttaattcaa ataagctgat tttaatgacg |
| 109441 | ttttcaactg gtttttaaat attcaatatt ggtctgtgtt taagtttgtt atttgaatgt |
| 109501 | aatttacata gaggaatata ataatggaga gacttcaaat ggaaagacag aacattacaa |
| 109561 | gcctaatgtc tccataattt tataaaatga aatcttagtg tctaaatcct tgtactgatt |
| 109621 | actaaaatta acccactcct ccccaacaag gtcttataaa ccacagcact ttgttccaag |
| 109681 | ttcagagttt taaattgaga gcattaaaca tcaaagttat aatatctaaa acaatttatt |
| 109741 | tttcatcaat aactgtcaga ggtgatcttt attttctaaa tatttcaaac ttgaaaacag |
| 109801 | agtaaaaaag tgatagaaaa gttgccagtt tggggttaaa gcatttttaa agctgcatgt |
| 109861 | tccttgtaat caaagagatg tgtctgagat ctaatagagt aagttacatt tattttacaa |
| 109921 | agcaggataa aaatgtggct ataatacaca ctacctccct tcactacaga aagaactagg |
| 109981 | tggtgtctac tgctagggag attatatgaa ggccaaaata atgacttcag caagagtgac |
| 110041 | tgaactcact ctaaggcctt tgactgcaga ggcacctgtt agggaaaatc agatgtctca |
| 110101 | tataataagg tgatgtcgga aacacgcaaa acaaaacgaa aaaagatttc tcagtataca |
| 110161 | caactgaatg atgatactta caatttttag caggtagctt tttaatgttt acagaaattt |
| 110221 | taattttttt ctattttgaa atttgaggct tgtttacatt gcttagataa tttagaattt |
| 110281 | ttaactaatg tcaaaactac agtgtcaaac attctaggtt gtagttactt tcagagtaga |
| 110341 | tacagggttt tagatcatta cagtttaagt tttctgacca attaaaaaaa catagagaac |
| 110401 | aaaagcatat ttgaccaagc aacaagctta taattaattt ttattagttg attgattaat |
| 110461 | gatgtattgc cttttgccca tatataccct gtgtatctat acttggaagt gtttaaggtt |
| 110521 | gccattggtt gaaaacataa gtgtctctgg ccatcaaagt gatcttgttt acagcagtgc |
| 110581 | ttttgtgaaa caattattta tttgctgaaa gagctcttct gaactgtgtc cttttaattt |
| 110641 | ttgcttagaa tagaatggaa caagtttaaa tttcaaggaa atatgaaggc acttcctttt |
| 110701 | tttctaagaa ggaagttgct agatgattcc ttcatcacac ttacttaaag tactgagaag |
| 110761 | agtatctgta aataaaaggg ttccaacctt ttaaaaaaga aggaaaaaac tttttggtgc |
| 110821 | tccagtgtag ggctatcttt ttaaaaaatg tcaacaaagg gaaaataaac tatcagcttg |
| 110881 | gatggtcact tgaatagaag atggttatac acagtgttat tgttaaaatt tttttacctt |
| 110941 | ttggttggtt tgcatctttt ttccatattg ttaattttat accaaaatgt taaatatttg |
| 111001 | tattacttga attttgctct tgtatggcaa aataattagt gagtttaaaa aaaatctata |
| 111061 | gtttccaata aacaactgaa aaattatcat gagatgtgta tttaaacttt ttcatgaaca |
| 111121 | ttgcttatat aatcattcct tctgtcttaa tgtactacat ggtcttagcc ctgttcctat |
| 111181 | aggattatca tgttctctgc attatagagc cacctaagat gtactttttg ttaaatgact |
| 111241 | catgctggaa tatctggatg gggagatgtt ctttcctaat gtagtcatgt gccacaaaat |
| 111301 | gacgtttcgg ttaacgatgg atcacatata tgatgatagt cccatgaaat tgtaatggaa |
| 111361 | ctgccctata caggtgtacc attttttatc ttttatttca gatttttact gtaccttttg |
| 111421 | tatatttaga tgtgtttaga tacacaaata ctttccattg tcttacaatt gcctgcagta |
| 111481 | ttcagtacag caacatgctg tacaggtttc tagcccagga gcaataggct ctaccatata |
| 111541 | tctaggtgtc tagtaggcta ttccatctag gttctcgtat gtataacctg ggatgtttgc |
| 111601 | acatcgatgt ggtcacctaa agatgcattt attggccagg cgccatggct cacgcctgta |
| 111661 | atcccagcac tttgggaggc cgaggcaagt ggaccacctg aggttaggag tttgagacca |
| 111721 | gcctggccaa catggtgaaa ccccatctct actaaaaata caaaaatcag ccaggtgtgg |
| 111781 | tggcacacac cagtaatccc aactactcgg gaggctgagg caggagaatt gcttgaacct |
| 111841 | gggaaaggga ggttgcaatg acctgagatt gtgccactgc tctccaacct ggacgacaga |
| 111901 | gcgagactgt ctcaaaaaaa aaaaaaatgc atttctcaga acttatcctc attgttaagc |
| 111961 | aatgcatgac tataatctgt tgagagaggg atgaaatcac ctgtagttat agcgctttaa |
| 112021 | gataccattt gaaaaggtta cgttttcctt ttctttgaca cggttagctg tctgaaatac |
| 112081 | agtcaatttt aaccctaatc tcttaatatc aggaatgccc ttacacctac tttggagtgt |
| 112141 | ctggtgcttc gatatagttg catgtaatgt gctctcatct gtttttacct gattcctgct |
| 112201 | cagttcttca catggcatat tgtgtaactc aatctatatt taaaacttgt aagcatccga |
| 112261 | attatttgtt tatggtagaa ctttttactt gcaagtcgtg gtaggagtgt ttgtagttgg |
| 112321 | tactaaaatg tgatgacttg gaagaattaa ttaatgacct atatttgggg actttaattg |
| 112381 | gatgctatag ctgcaatgag aatagaacca gagaactctt gatatgcaag gttattcatt |
| 112441 | ctgtgataat aatgagagga atattcgatg tctctttgag tcagttttcc ttccgatcac |
| 112501 | ttccgcattc tgcagtgaca caatccttaa tcatagcttt cattacaata ttcctttact |
| 112561 | ccagacctca aagactcctc actgcctcca gcatcaaatc taaactcttc tacctggttt |
| 112621 | tcaacgccct acgtaaactt ttcctccttc attccctata gcttggtttt ttttttttta |
| 112681 | tcactgccac tattatctat cacaaatgtc aatcatgacc ataccttgct taagttggtt |
| 112741 | tcccttgcca agagcactgt ttttcctata cctgttgaaa ttttggaaat ccaattctac |
| 112801 | ctcctctctt cccttaagca tctcttcctt cccttctccc caaatttata tttagtcaca |
| 112861 | tatattatcg tactacctac taatcattcc atgtgttttt ttacgagctg taagttctga |
| 112921 | aggcaaccat gccttgtaca gtgtccactt agggttctga ataattaatc atctccccaa |
| 112981 | aatctgaaag ccttctatat accaagcaaa tttgtttagt tatgcagcaa aactcaaatc |
| 113041 | tataaaatca aaaaggaata aggaaataca gattaaacag ttgcagcaaa gactggtgat |
| 113101 | cttaaggtat ttagtcaaag ctggtggtag aacaaaaaca gtagtcttac agattctacc |
| 113161 | tcttgattaa ctcagtggct aattttgcct tttctcaaag ttcttttgca agaacataaa |
| 113221 | gatatttttg tttctttagt tgagtgctgt aactttattc ctttgtgttt ctcataagta |
| 113281 | tgatttggca gtctgccata cgttttttgt tttttttctt cctctttgag acagggtttt |
| 113341 | gctctgtcac ccaggctgca gtgcagctgt gtgatcacag cttagctcac tgcagactta |
| 113401 | gcttcctggg ctcaagcaat cttctcacct cagtctcctg agtaactgag actacaggtg |
| 113461 | caccccacca catcccgcta aatgttttaa tttcttgtgg agatggtgtc ttcactatgt |
| 113521 | tgctcaggct ggtcttcaac tcctgggctc gagcaatcct cctacctcag cctcccaaag |
| 113581 | tgttggaaag tgttgggatt acagacctga gccaccacac ctggcctgtt ataagttaat |
| 113641 | acaataattc atcaactaac ttaaagaaca ctaagactct tacaaaagta ggtatgagtt |
| 113701 | ttagtaaaag tctcaaaaga taaactgtca cttaaggaaa actagagaac atatcattgc |
| 113761 | caaatggtgt ttttcagaga ttataccatt caacgcccac atgctgaatt gggccattca |
| 113821 | ttataactcc aggaacatgg caatcagtaa gagcccacat gtttctttga atacaccgta |
| 113881 | agtgaaagaa tataaagtag tctagttaat attatgttta atcaaggagc acattcctaa |
| 113941 | agatgtttgt tcattcattc tacagacatt tttcagaggc ctgctaagag tcaagcatta |
| 114001 | tgatagacgc catggataaa agcttacaag tcaaccagcg tgggtataaa taatgtgact |
| 114061 | agcactagaa caggtatcat gatggggatt ctgagtataa atattttttt aaaataaatt |
| 114121 | tccccgtgtt atttttggct tttacctccc taatttaggc tttctaaatg gcacagcatt |
| 114181 | tctgaggatg caaacacctt tctacagagc aaaaacagca tttgtataaa tttgtgtctt |
| 114241 | tggggaacca agagacttta aatgtgttta aaccaataat tcagtcaata tcaacattag |
| 114301 | cttacatgta atattctctt gatagcccaa ttttttaaaa cactgtattc ttagaagttt |
| 114361 | ggtttctaag atgtcacttt aagctctttt gcttgttgct tttgtgggat ccacaaattt |
| 114421 | tgttctcagg tacataaatg aaggttagta tagaggataa atattatgat tcttatctgg |
| 114481 | gaaagacagg tgctgaggtg taaaagagag gatcctcgcc acccatgccc cgcaccccct |
| 114541 | cgccccctgc acccacggat gtgcagtctt acctgcgggg ggaaaggtct ccgagcctgg |
| 114601 | cctgctgctc cagctcaggg ttcccccttt catgatggct ttcaaagatt tcttcactct |
| 114661 | gaagtgaaag aaattttggt aagatttgat attgtaggga cctcctaatc tatatttttc |
| 114721 | tctctcccaa tttctgtgtt tgattttggt tttgagtctc tcgataagca aaatatccag |
| 114781 | tttctcatgc cgctttctca ggttttcccc agccactctg gatccattat ggtttgccct |
| 114841 | ttttggcttc ctttaggcac actaaaaact ccttcccaaa agcaggtatc ggccgggcgt |
| 114901 | ggtggcaggc gcctgtaatc ccagctactc tggaggctga ggcaggagaa ttgctcgaac |
| 114961 | ctgggaggcg gaggttgcag tgagccaaga tcacaccatt gcactccagc ctcagcaaca |
| 115021 | gagcgagatg ccatatccaa aaaaaaaaaa aaaagcaggt gtcctttccc cttaatcatg |
| 115081 | aagggctatt catactttac tgccccaccc ctattgattc ataagaggac agtaaagcga |
| 115141 | tcactgcatt caacatctcc tttttttttt tcttgtaaga aatcaaggtc tggaaaagtt |
| 115201 | gcactcgccc tgagaccaga aagtgctgga ggcagagatg aagtaagccc agtgtaggct |
| 115261 | ttcacagatg cgtgataaca agtctaacta aagaagtacc tgggatacta gttttgccaa |
| 115321 | ttcagctcta aaacaatatg gcaatcttat attccaaata tatatatata tatttgtatt |
| 115381 | tatagtagag atggggtttc accatgttgg ctaggctggt cttgaactcc tgacctcaaa |
| 115441 | tgatccactg acctcagcct cctaaagtgg aggaaacata tatatatgtt ttccttttaa |
| 115501 | aataggatgt cagtccaata agaatctaaa ttttagttcc ctctaatata tatatctgac |
| 115561 | tagggaccag ataatatttt tcatgtgtca atatataaaa gttggccagg tgcagtggct |
| 115621 | catgcctgta atcccagcac tttaggaggc tgaggtcagt ggatcatttg aggtcaggag |
| 115681 | ttcaagacca gcctagccaa catggtgaaa ccccatctct actaaaaata caaaaattag |
| 115741 | ccgggcatgg tggcaggcac ctgtaatcca gctatttggg aggctgaggc aggagaatca |
| 115801 | cttgagcctg ggaggcagag gttgcggtga gctgagattg caccactgca ctccagtctg |
| 115861 | ggcgacacag tgagaccctg tctcaaaaga aaaaatatat atatatatat attttttatt |
| 115921 | atattgttta ttaaacaaat aaaaataaaa gatatctcat cagttacgat gtaaatgaaa |
| 115981 | aaattgtgtg tgtgtgtgtg tgtgtgtgtt atgatgtact tcttgtgagt tgtggtcagg |
| 116041 | ctttgaaagc cactatgtat gtgtgtgtgt gtgtacataa aagtaagtat cagtcccagg |
| 116101 | attcaaattc gaaccagtcc cagtcaaatt caatctaatt attttcacca cactaccaaa |
| 116161 | agtctttctt cacattttct atataaagtt gaactaatta atacagcagt gaatgttaca |
| 116221 | ttgtatcctt ttgcagtttc tcatctatga cattactatg cctgaattcc ccactggact |
| 116281 | ttgaactcag tcttactcat ctttggatcc ccagcgtgta atacaggccc ttcataaaga |
| 116341 | gtgaccatta ttactaacaa aatttcctct cactgtgaaa cctgctccca attataaaat |
| 116401 | gaattgtgct ctttcgaact ccgttgatct attgtatgca gggtggatca tagagtctta |
| 116461 | tttcataata acctagtgat ggaaaaataa caatgattca ggagtaggta atggaccttg |
| 116521 | tcatctttta cactgaatga tgagatttcc ctaatttata gattttgtca tgcatgagat |
| 116581 | gccctcagga gcttgcagaa atgccttggc acgttgctct gctgacatgt gtcagatggc |
| 116641 | tggtgtggag gtagagggag tctcctctgc caagcaagtc taatggaata taacactggt |
| 116701 | gccattgagg atgcaatgag aaggacccac agcagatcca gagactgcat tcataaaagc |
| 116761 | tgcacagtat accatgtttt attaaggtat agacagatta gttgtgttct agcatagtgg |
| 116821 | ttttcaaagc ctgaccacaa ctcacaagaa gtacataaca cacacacaca cacacataca |
| 116881 | cacacacatt ttttcattta catcgtaact gatgagatat cttttatttt tatttgctta |
| 116941 | attttttatt tctttttttt ttgagacgaa aggtgaattg tagccaaagt acttttagga |
| 117001 | aattttaaaa ttacacattt tgaaagctct aggaggaaga gagcatattc aagtaaaacc |
| 117061 | tttcttttat tcaaatagta gactaaaaaa ttgaccatag actaactcag caattgctgc |
| 117121 | catatttctt agtgagggat tccttatgag ctccattgaa tgataaaagg atactcttcc |
| 117181 | aaatcagagc aacaatcgtt tcttgtgaca ctgcatcctt tttcccttcc ttcaccctga |
| 117241 | attgccccta gggtatacct aggagaggga gggctggcaa ccagctgctg ttgtgaaagg |
| 117301 | gatgtcattc atggcctgcc ctattgggag gccaatcagg cggaacagcc cctctcccct |
| 117361 | tatggtcatt ttatcctgaa gaaaaagggt ggcaaaaaga tggcaaaaaa gattgctgat |
| 117421 | cacagatcac tgttacaaat acaacaattg gctgggcatg gtggctcacg cctgtaatcc |
| 117481 | cagcacttcg ggaggccgag gcgagtggat cacgaggtca agagttcaag accagcctga |
| 117541 | ccaacatggt gaaaacccgt ctctactaaa aatacaaaaa ttagccgggc atagtggcgc |
| 117601 | atgtctgtag tcccagctac tcaggaggct gaggcaggaa agtcacttga acccggaggt |
| 117661 | ggaggttgag tgaaccaaga ttgtgccact gccctccagc ctgggcgaca gagcaagatt |
| 117721 | ccgtctccaa aaaaaaaaaa ggagggggtg gcaatgagac agggaactgc tctgggtcca |
| 117781 | actcccctca catagcccca acccccatat ccaccccaca aacctccacc tccccacttc |
| 117841 | cagcctctcc agtttcaaag tgacgcttac caacacgcag gctctccagg agcaccttga |
| 117901 | aaggtaactt cttacatctt tccaaaacac ttataatcta attgttcttt ctcccctata |
| 117961 | ttttgaagaa atgcatgctc tcaaaattga aaaaaacgcc tagaggaatt ttgtaaaaaa |
| 118021 | attatatttt tccagtttct ccaaaggaca tataaaccct ttttaaactt agattttgaa |
| 118081 | aagtcccaat agctgatttt caatcgatta ttgaaattgt tttttttttc ttccctaaag |
| 118141 | gggagatacc aacctttgaa caaaattaaa ttaacttttc cccaaagtta aatgatttta |
| 118201 | ctttgtgatt ttaggaatgt gtagaaagtg atttcagttc caactctttt aaagcaaggg |
| 118261 | tatctgggcc aggcgtggtg gctccctcct gtaatcctag cgctttggga ggccaaggtg |
| 118321 | gggcagatca cctgtggtca ggagttcgag atcagcctgg ccaacataga gaaaccccat |
| 118381 | ctctactaaa aacacaaaat tagctgggta tagtggtgcg cgcctgtact cccagctact |
| 118441 | cgggaggctg aggcaggaga atcgcttgaa cccgggagac ggaggttgca atgagctgag |
| 118501 | atcacgccac tgcactccag cctgggcaac agagtgagtc tctgtcaaaa aaaaaaaaaa |
| 118561 | aaacaaacaa gcaaacaaac aaacaaaaac aagggtatct ggtaatttaa ggtgaaagta |
| 118621 | ttattcacta ttataatcaa ttttcttaaa aacaggagct attaggccca attctgagga |
| 118681 | gagaaaaaaa agcagatttc aagggcccgt gccaccttct ttgtttaaag catagagaat |
| 118741 | gataataaaa tgttaaaggt tcagcatttt ctggcattgt aaatttaatt aattaattat |
| 118801 | gtaaattaat tatggaaatg agaaataaag tgaaaaggta tgtcaagtaa gaatcaataa |
| 118861 | ttatcaaaac gttctctacc aaatgggatg caggggagaa gggtgagaag aagggaagga |
| 118921 | aataaaggca aaggcatttg cccacagcct cagtccaggg tgttctacat gacgcagccc |
| 118981 | cagagtaaaa aatactagcc acaggaatga gtggctctgt cttcctctcc agaactgacg |
| 119041 | ggcaaaagag ggggaacctc accttttttt tttttttttt tttggaaact cactgggttt |
| 119101 | tgatatggag aacaggaaga gaatctaccc tagcagcaca ttcacagatg gtttgtctgc |
| 119161 | cctatttgct ccatctccct ggctcttcct agcacttctt ttcctacttt ccattttcat |
| 119221 | tggccgcaaa agccacttaa atattccttt ggaaaactgg gcagtctgtc ccctttttaa |
| 119281 | aaccacaact atttccagga gagttaaagg gctttcggac tgcccatcat tcatgcattg |
| 119341 | tggtgaacta gctcagactg cctctacctt tagtccagat cagctgcctt gggagggcga |
| 119401 | ttcctggagg aaaggatgag gagaggaagg ttttaggaca tcgtatggac tgaagccccg |
| 119461 | ttggagggga agggcaggct tgaggaacaa gcctcagtgt tcctgaggct ttgtggaaat |
| 119521 | gagaaatgaa tagagaaaga taagagactg gatcctaaat gcaattttgc ctatttcaaa |
| 119581 | attttcacag ggtctttttt tttttttttt tttgagacaa ggtcttgctc tgtcacccag |
| 119641 | gctgaagggc agtggtgtga tcatagcttc ctgaagcttg cagcctcgaa ctcctgagct |
| 119701 | caagcgatca tcctgcttca gcctcccaaa gcgctgggat tacaggcatg agccaccacc |
| 119761 | cccagcctca gggtcttttt aatccctaag atcttaaatt tcccagctac tattgttagt |
| 119821 | aatgagttca atttacttac tggttaaatt ggcttttatt attattatta tttattttat |
| 119881 | tttattttat tttatttgag acagagtctt gtgctgtcgc ccaggctgtg gtacagtggt |
| 119941 | gcaatctcag ctcactgcaa gctctgcctc cctggttcat gccattctcc tgcctcagcc |
| 120001 | tcccgagtag ctgggactac aggcacccgc caccacgccc agctaatttt ttgtattttt |
| 120061 | agtagagacg gggtttcacc atgttagcca ggatggtctc gatctcctga cctcatcgtc |
| 120121 | tggactaata ctctaaacgc tattggcaat agtttgttta caggaaaaac atcttcttat |
| 120181 | aaagctgact gcaaatgttt taataaattt gcaaatatgt aattctgttt aaaatatcag |
| 120241 | gaagtgagaa acatgttatg tgataactct ttcccattcc cgaaccaaga aaatgtaaag |
| 120301 | gcagtaagtg tgccaaggac actgaaagga agctgcccgt acatctttga atcttctcag |
| 120361 | gctgtttggt ttcatctgat tttaagctcc atggataaat ttcattgtaa caatttttat |
| 120421 | gtctgtaaat cttaacccat aaataaaatc acaaatcaga aaagtacttt attaggccag |
| 120481 | tctttgtcca agcaaatttg gtcccaaagc tagtttacaa ataattgcca cacagctaca |
| 120541 | aggccagtgg gttgactatc gtagcatcaa tggtgtgggc agggctgcag cctctggggc |
| 120601 | agcattagcc ccaactgaca gagggtatag gtgctcttaa caatctatga gaccccccac |
| 120661 | aacctggact cagctgtcat aagcctttac ttcttatgtt cttcctaagt actttatcgg |
| 120721 | gccaacaaat tcatcccatg aggaatccaa gatggaaacg tcaaaactat ctttggtatc |
| 120781 | atgcttcctc cccctacctt ctctgccttg atcccctcct tcattctaca cattttctct |
| 120841 | gatagtcgtg ttctagccac tagggagaac gtttcagtca atgggtatga tttctgctca |
| 120901 | cctctttttg ccatagctat tagtaagctc tcaccactga tgtctcagct gactgtgaca |
| 120961 | actgccagcg gctggtcagc ctgcctgcct tcatctaacc tgctctttac tgtctaccag |
| 121021 | agtcagcttc gtaatacaca ggtgccctcc tcctcctgtc aacattcctc aaccaaagcc |
| 121081 | tcagtaaact gtctctcatc taccttccca gcattcttga gccattccca ggatagccta |
| 121141 | ggccctttag acctctgaga tgttataccc ttcctcattc tggaatgccc ttcagtgtct |
| 121201 | tacccacctt tcagagttct atttattctt tttttttttt tagatggagt ctcactctgt |
| 121261 | cacccaggct ggagagcagt ggcaccatct tggctcactg caccctctgc ctcccaggtt |
| 121321 | caagtgattc tcctgcctca gcctcctgag tagctgggat tacaggtgcc cgccaccatg |
| 121381 | cccagctaat ttttgtattt ttagtagaga cgaggtttca ccatgttggt cagtctggtc |
| 121441 | ttgaactcct gaactcaggt gatccaccca cctcagcctc ccaaagtgct gggattacag |
| 121501 | gtgtgagcca ccacgaccag ccttatttat tctttagagt tgaattcaaa tgccacctcc |
| 121561 | tttgaaagcc atccttgatg tccttgtgag gaattacttt ctctaccaca ttatatattc |
| 121621 | cttttactgt agaaacgtca ttctgcttta tattataaat tattagaaat atgttgtttc |
| 121681 | ccccatgaaa gtttcaagtt tcctgagttt aggcctgatg atacaatcat tttcatatga |
| 121741 | ctcacagcaa ttgccttagt atatattaaa ttgagctgaa taagccatgt acccagttga |
| 121801 | acacaccaaa tattttagtg atgtcatttc tttattggtg aaagagcaag gccaacgttg |
| 121861 | atcttaatgt tattctctct tttatcttat tcagctccct caacaaaaat acattctcat |
| 121921 | agattaatgt aatcaccacc tcactcctct caactttaga ccctatacta gcctgagaaa |
| 121981 | tccagatcca acttaatgag ttctccttta tcccaagtcc tcatgaccta taaatgtaac |
| 122041 | ctctaggaca atgttacaaa gagtgagatc tttataccac ctgcattctt gtcaccaaac |
| 122101 | gtgtaagtta aaaattcaga ttctgagtgt ggtgactcac gcctgtaatc ccagcacttt |
| 122161 | gggaggctga gccaggtggg tcacctgagg tcaggagttc gagaccagcc tgggcaacat |
| 122221 | ggtgaaaccc tgtctctact agaaatacaa aaattagcca ggcatggtga caggcacctg |
| 122281 | taatcccagc tactctagag gctaaggcag gagaatcact tgaacctggg aggcggaagt |
| 122341 | tgcagtgagc tgagatcact ccatggcact ccagcttggg caacaaagtg agactctgtc |
| 122401 | tccaaaaaaa aaaaaaaatt tagattccag gacccgaccc tacacctccc taattagaat |
| 122461 | ctcagagagt ggggccctgg gaatctgcat aattcataac cttctcagag ctctggtaca |
| 122521 | caagaaacca tgagaatctt tgttctggaa acttcagaga acttggctga gggccacccc |
| 122581 | aggatttggt ggcgtccctg aatctcccat tagctctgac actgactaga cttcaaatat |
| 122641 | cacagaaggc aagcattgaa gtgtgtttat attcaaatag ttttcttgtt gagaatcaga |
| 122701 | aatattaata aaacttttgg gagtcaaagt aatgagaaga caaggaactg aacttgcccc |
| 122761 | ctgatctgtt atcatttggc agtaaggtac atattcaaca ataggataat tctgtataag |
| 122821 | acctgaatct gaccttcttt catctttcac actgacaatt ctgtctctaa acacaggatt |
| 122881 | aaatgaggaa ctaaaattca ccaagatcct gctaggtgtc aggtacagtg ctagctgctt |
| 122941 | ccacacattt ctcatttaat cagccacctg tgaaattgct atgatttatc accatttggc |
| 123001 | agaaaaggac atttagggca caagagatta aataagctgc tggttgggta cattaagtat |
| 123061 | taataaataa tcagatagat aagttaggta gttaagtaag ctgccagatt agtaaacagc |
| 123121 | tagtttcaaa tcccagctcc aacatttact agttgtatga cccttgggga aactcaacct |
| 123181 | ctgagtctca tggcactgct gataaaacgg aaaacatcat cctcacaagg ttctgaggac |
| 123241 | tagggatgga ctcaggtaca cacagcaccc agtacaatgt ctggccttca gccagtgata |
| 123301 | gcaacccttc actcacctct ccattccctt ccagcccctg cattcaaaaa cttttattta |
| 123361 | tttatttttt taatttagga ggctctttgc tcagaatcct gaaaaggctt ttgttacttt |
| 123421 | attttttctt ttttttcatc ttctgggagc acagaatagg cttctgctcc tttaaataac |
| 123481 | tttaacaggc accaaaggaa cactgctagc tctttcttaa ttctgtaggt ccacatttag |
| 123541 | gaaaaagaaa ttgtcagcct ctgacttatt tccagcttac aaaaaggctg tgatgttggc |
| 123601 | agcccgggga aagcagttcc cgtgagtcat gctttcacct ttcagccagt gaagaacagg |
| 123661 | aaatagtcac atcactattg ccaacaagca cagcgaatcg cttccaccac cgggctttcc |
| 123721 | cagatgacgt cagtgagcca agtgcagggc atcacccttg ccagatggcc ccggaagagt |
| 123781 | ctcagctgcc ctgtcaagtt tagcttctca agctccccag aaccagcatg gcaaggatca |
| 123841 | cccctccaga aaaggaaatg atttctctga tcatgatcaa accatgtcat aattttagct |
| 123901 | gtaggtggtg agtatcagtg actgtattat agatggtttt ggttcacagc tatttttttt |
| 123961 | ctcggtatat ttactctcaa gggcaaaggg gtggcattta tcacaatgct atgattcact |
| 124021 | ctactagact ggctgggttt catttcatct ttgagttcta gacctaaggc caaaaggatg |
| 124081 | tcagaatcgc cacccagagc tagatgcatg ctcaatgcaa cctggccatc tctctcggac |
| 124141 | agtggccact aaacacaagc tgcaatgatt attgttgatg ttagtcaatt gatttgtccc |
| 124201 | ccttttaata atccatgatc cacacagaca tgaatctaaa cctttttttt gaacctatct |
| 124261 | ttttcttact agttttctta catattttgg aataacaatt tccctggtta taatacacaa |
| 124321 | tgtgtgaaac actaattgca tttattggtc ctaaattcat tcccaagtct aggatttggg |
| 124381 | gatttagtgc acaaatccac tttcttcctg ctcatgttgt tcatgacttt atggattctg |
| 124441 | cttacatttc ctcccaaagc cttggtcttt ctaagcagaa atctgaccag cctctgttcc |
| 124501 | tactctgctt cctccaccac cttgactgtt aacatacact gttgtgggag aagcttttca |
| 124561 | ttaaatacgc aaacaaatca acaataagga agaaaacaac tgaagcacag aaggatgatt |
| 124621 | gtaacatgga cttgtgcttg tttaacagta cccatactgt ttggtgagtt gcctaaatac |
| 124681 | agagggagat ggcaacttaa caggatgggt ttggagtctg aattaaataa acaacatggc |
| 124741 | aaaagaagga agaaaccaaa agttgggtga ctagggcttc ttggattcta ctcaacactt |
| 124801 | tccacaccta caggcctctg attctatccc caccgctgct cctacactgt ctatgtttct |
| 124861 | cttttacaac aacaacaaaa aaatagcact tgtttactgt tcacaattat tgaaataaat |
| 124921 | cactctaatt tggattcctt tatatgagaa cttccatatg cttaaaactc ccgtagcatt |
| 124981 | tttatagtgt acaggtcata aacatgttgt ctgaagatgg gcaaagtagg tcgtagagat |
| 125041 | aggaagtgag ttcacatcac ggcgtgagtc aaggcagcag catatgagct caggggccaa |
| 125101 | atctgagctg tgatcatgtg acacagtcct caccttcaga gatgacattc tcccagaagt |
| 125161 | ccagggtgca gaaatggata ggctgatgtt cacttttgaa gaggaccaca atgaaaccct |
| 125221 | tttaatatta ctcaatgatt ccacagacct cctgggtgtt aaatcaggtg aatctatttt |
| 125281 | taaaaattgc tatttacttt tttaggtggc tgatcaggac agtatgaaat ttcatacagt |
| 125341 | ttcctaactt gagaaaacat ggtgatgcaa ttcctccaac caggagggat ttatggacag |
| 125401 | cggtggctac gtcctaatct gcccagaata caggatgact aaacaaattg gcaaacggac |
| 125461 | gcagctttct ctctctctaa gaaaagtctg ctgagaaccc tcgttcccac tctgtttctg |
| 125521 | cctccaagaa gaaaagccta aaactcactt tcttccggac tctttcaagg tcagtggagt |
| 125581 | tcctctgggg ttcatattca gatactttgg ggattgatga tggatcataa atgttgctga |
| 125641 | agttcatttg agcccatggc ctctggtttc agaaccattc agccagtcaa atatttaaat |
| 125701 | tttagtgagg caggggaaag ggaacccctt cgggggctgc tatacagcaa cttatattca |
| 125761 | caattcacaa ttaaatacac ttcagtttct aaatatatgt tactttaaaa ttatacagtg |
| 125821 | cttagcaatt ttcaaatttt cattttgtgc tcaaaatatt ttcaggaggt agctgttgtt |
| 125881 | atacccattt gtacaagcaa gaaactgagg tttcaagagg ttatgttgct taaacccaga |
| 125941 | tctgcctgat tccaaacctt atgctttttt taaacttcta ttttgaaata attatattaa |
| 126001 | gtcacatgag gttgcaaaga aatggaaagt ctcatgtccc ctttctccaa acctcatcca |
| 126061 | atgttaacat ctaagaaatt gacactggca caatccacag agcctattca gggttcacca |
| 126121 | gttattcacg cacttgtgtg tgtgcatgtg tgtgtgcagc tctgtgcaat tttgtcatgc |
| 126181 | tctttttttt tttttttttt gagacatagt ttcgctctgt tgcccaggct ggagtacagt |
| 126241 | ggcacagtct ctgctcactg taacctccaa cttccagttt caagcaattt tcctgcctca |
| 126301 | gcctcctgag tagctgggat tacaggcatc tgccaccacg cccatctaat ttttgtattt |
| 126361 | ttagtagaga caaggtttca ccatgctagc cagactggtc tcaaactcct gacctcgtga |
| 126421 | tcttcccacc tcggcctccc aaagtgctgg gattacaggc gtgaaccacc gcgcccggcc |
| 126481 | tgtcatgctc tttttataca tgaaccatac actgttctgc caattttaaa tatgaaggca |
| 126541 | gattacagaa ataaataaaa tgatttttct tttactacaa cagtatctac caataatcac |
| 126601 | atacatgacc aaatagcttt cacttctagc tgccgttaag aagaaagaaa aggatgaaaa |
| 126661 | gaaaaaaaat cctataaacc ccagttcttg aaagcattag tctgtgctat gtgccttagg |
| 126721 | tacagattaa ggaaacacaa tttgttgatt tattgataat tgtgacagca atcttccctc |
| 126781 | ttgtcaggaa gttctataag taaaataaag gtaattttac cttgacttca Ratttagtct |
| 126841 | catcatcttc ctttccccgg gagttcaact ctgtctccgt ataaggttcc tcagacgttc |
| 126901 | tcagaggacg agctagaggg actaagagaa cctgccatta gtgtggttat ttaacttata |
| 126961 | aactataact cagtgcactt tgtctgattt ggacaaatag ctggaccacg tcaatgtggc |
| 127021 | taactataaa agctcacttg agctgctgcg ttgatttcct agggctggat gaaattggag |
| 127081 | gtgaaggaga gacacagagg agaaatagaa tccttgactc cagggacttg acatttcagc |
| 127141 | agaagagaaa aaagtcacac ccagcaccat atgaaacatt aaactacaac aaatctgtac |
| 127201 | atatagcaat gtggtaaaga atagtagatt ttctgtcatt cccattttta aggcccagct |
| 127261 | aaaaggtcac ttttggctgg gtttggtggc tcacacctgt aatcccagca ctttgggagg |
| 127321 | ctgaggcggg cagatcacct gaggtcagga gttcgagacc agcccggcca acatggtgaa |
| 127381 | atcccgtctc tattaaaaat acaaaaatta tccgggaaca gtggcaagtg cctgtaatcc |
| 127441 | tagttactcg ggaggctgag gcaggagaat cacttgaacc tgggaggcgg aggttgcggt |
| 127501 | gagctgagat tgcgccactg cactccagcc tgggcaacag agctagactc catctcaaac |
| 127561 | aaacaaacag acaaacaaac aaaaaggcca ctttcctcat gaagtcttcc atattacaaa |
| 127621 | ctttatggat tcttttatgc actgctgctt tggcatgcat catgtgtaca gcataatgtt |
| 127681 | gcacttgatg ttgttcttgg attcaaagac taaattctag gtactacatt gtattgaata |
| 127741 | tattttccat tataaaggta attaattttt gcttattaca gaaaatctga aaaacactaa |
| 127801 | actcttaacg ctgaagcaca atcacttagt atatttcctt acagtcttct ttcaaatgct |
| 127861 | ctccctcttt ttacacacat acatgtacac acacaatcat actatgattg tattgtaaga |
| 127921 | ggatggccat gcctgctgct cttgctcttt tctcttggcc actctatctc ccttctcccc |
| 127981 | acctgggaaa tatcctttct tcaagctcca tggccatttg gtagtaaaat tgggacttga |
| 128041 | gaggagaagg cctcaaggct tcctaaccca ccttaccagc tttgccaagc attgtgggtg |
| 128101 | atggccacaa ggctaataga taagaggtac tttgaattct tatttgtcac agtcaccacc |
| 128161 | cttgcatatg ctggtccctt gggaaactca caggagacat gattaatccc aggcaggatt |
| 128221 | tgagcatttc tttctttctt tctttttttt tttgagacag agtctcgctc tgtcgccagg |
| 128281 | ctggagtgca gtggtgtgat ctcagctcac tgcaacctcc gcctcccagg ttcaagcgat |
| 128341 | tcttctgcct cagcctccca agtagctggg actacaggcg cgcaccacca tacgcagcta |
| 128401 | atttttgtat ttttagtagc gacggagttt taccacgttg gacaggatgg tcttactctc |
| 128461 | ttgacctcat gatctgcctg cctcggcctc ccaaagtgct gggattacag gcgtgagcca |
| 128521 | ctgtgcccag ccaattttag catttgtagg tcccaagacc aaaatgccat tcattgaatg |
| 128581 | ccaccaatat atcaccctga ttcttagcta tggtagactt gatggaggtt gaccatctga |
| 128641 | tctccagagg ccccttccaa cccttccatt ctgtgagcca actggaagca gcttgggctt |
| 128701 | tctatgggtt tttcatagat gttatgttga aaatcgcaga aaattacact gctaccccag |
| 128761 | gttatacata ttaccattaa tgctgctttg taataacaga caatcctttg ggctcctccc |
| 128821 | tctctgtggg atctctataa agtgagtgat tccaggcaca ataactagat gctagaaatg |
| 128881 | catgcactat aatttgtgac accatgactg agaagcgcct gtccttgaca ctgtaagtag |
| 128941 | agtgaagcag tgaaaggcgg ggccacagaa gccacactgc cagtgttcaa accttgctct |
| 129001 | tccatgcacg agacaaacat gattacagtc acctctgtac cttacctttt tcatctgtaa |
| 129061 | agtagggaaa aatgagaaat cctacagtaa agggctgtga taaggcttta atgagttcat |
| 129121 | ttgtgaagaa tgtagatcat atacaatgca taacacattt tagctattat tgccacatgg |
| 129181 | cactaaaaag ttttttcacg ttccttatga acttcagagg tgttagaagt tgtagccaag |
| 129241 | gcatccagct aacaagagtc agagctgaaa ttccagctct taagcccctc cacacatctt |
| 129301 | ctttatactg tgaaatacaa ttgcttatct tggcacagac tcagttacta aggaatcaga |
| 129361 | caaaaatgtt tgaaaatcct tttagtgact tgctggagtc cacactcagt agatacttac |
| 129421 | caaaaacaaa agtgcaggat tgttctgcaa aacgagctag ttgtttgact taggtcaatg |
| 129481 | acaacctgtt cagacttgac aaagacctat ttattctggg aaggagtgtt atctattatg |
| 129541 | aaatcttgta tttgaaaagc tgagaaacaa ccctgtaatt cctgctttat ttacttgaaa |
| 129601 | caaacatatg tacctggaaa caacgaatga ggccaagtca tagatgtgaa catagtttag |
| 129661 | cttgaggaca gacgaaaggt caagagaacc aagttctgtt tccaatcatc taagaaggat |
| 129721 | gtctctgtgt atgtttgggt atgtctgtgg tatgtgtgtg caagtgtgtg agagtgtatg |
| 129781 | tgtgtggatg tgtgtgtgtg tgtttgcaga taaaaattca ggaaagaaat acactaaatg |
| 129841 | ttatcaaggc ttacttctaa aaagtaggat acatggtgga agacaaggga tcgtaaattg |
| 129901 | tactaaatgg agaattacca aaacacccca caattttcat ataaacaaaa actgaaagaa |
| 129961 | tttattgcta acaggccagg catggtggct cacaactgta attccagctc ttagggaggc |
| 130021 | agaggcagga ggatagcttg agcccaggag ttcgaaacct gcctggacaa tatagcaaga |
| 130081 | ccccgttctc cacaaaaagg aaaaaataaa gacaagaaaa gaatttgttg ctagtagact |
| 130141 | ccccaacaat aaatactaac agaagctctt tattccgaag agaaatgaca caagatagta |
| 130201 | attcaaatct ataataagga atgaagaacc ccagaaatgg tgaataaggg ggtatgtaca |
| 130261 | cacacacaca cacacacaca cacacacaca tgcacacaca cacgtatgta tttctcttaa |
| 130321 | tttgatacat gactatttaa agcaaaaatt ataataccat agtgggggat taacatatat |
| 130381 | agatgtgata tttatgataa gaatagcaca gaggatggat ggggagcata tggaagtatg |
| 130441 | acattgtagt atgtagtatg atttcctata ttttgcatag aatggtatga aactaattca |
| 130501 | atagatcatg acaagttaag gatgcatatg gtaatcccca gtctggtgaa aaacagtaca |
| 130561 | aagagatatg ataacaagcc aatagaggaa ttaaaacaaa atattaaaaa atatttacta |
| 130621 | acccaaagga aggcaggaaa ggaggaaaag aggaataaaa agcacatgag acatgtagga |
| 130681 | taacaaaaca ggaactctaa atccaactat atcaataatg atgatggatg taaacataaa |
| 130741 | aatctaatca ctccaattac aaggcagagg tcacagtgta taaaaaggca agacccaact |
| 130801 | atatgctgcc tacaacaaat atactttaaa tacaaagaca gaagaataga aaacaaaaaa |
| 130861 | aatatgttct gcaaacacta agcatgagaa aactggaatg gctgtgacaa tatcgcacaa |
| 130921 | gatagacttt aagagtattt ctagaggtaa ggagggaagg agagacattt cataattata |
| 130981 | aaagagctaa tatatctgaa agacataaaa accatgaata tgtatgtgct taatgacaga |
| 131041 | ggtccacaat acacgacaca aacaaatgac atgtacatcc tgcccaaggg cagtttattc |
| 131101 | caaggatgta cagttgtttt aacatttgaa aacaaatcat tgtaatttac catattaaca |
| 131161 | aataaagaag aaaaaccata tcattctctc aatacatgga ccaaataagc atttgaaaac |
| 131221 | tcaataatca ttcacgataa aaactctcct aaaaaataga aatagaagag aattccctca |
| 131281 | atctgataaa agacatctgt ggaaaaccta tagcttacat catacttaaa gatgaaactt |
| 131341 | gaactctttt cctaatattg ggaaaacaca cagatgtctg ctctcaccat ttctatttga |
| 131401 | cattgtagtg gaggtcccag tcattataat atgacaagaa aaaaagtata aagattgaaa |
| 131461 | cagaaaaaag taaaagcatc cctattcaca gatgatagga ttatatttct atatagaaat |
| 131521 | ctattcctat tcttaattat atatggtaaa ttccattcat tatagggtta aaaaaatgtt |
| 131581 | aaatgcctaa ggcctttaca ctcaaaacga ctgcattgtt gagagaaatt aaagatgacc |
| 131641 | taaaaaacat aagttataac atattcagga attggaaaag tcaatattgg taagataata |
| 131701 | gttctctcca aattagctca tatatccagt gtaatcccta tcataatccc agcagaaatt |
| 131761 | gaaaggtgat tctaaaattt atggaacaat gaaaaggacc taaaatagcc aaaacaacct |
| 131821 | tgaaaaagaa caacgttgga acatcgcatg acttgatctt aaaggctgac taataagcta |
| 131881 | tggtaatcag gaccatgtgg gattggcata agaacttacc tattttttaa atctttgctc |
| 131941 | aaagaaccaa ttttactact ccattgctaa taaaacatgg gccttttaaa ggtcctgaat |
| 132001 | ggggtctatt ttatgctgtt attattatgt tattattatg atgcatttgt tgttgttgtt |
| 132061 | gttgttgttg agacagagtt ttgctcttgt cgcccaggct ggagggcaat ggcgcaatct |
| 132121 | ctgctcactg caacctctgc ctcccgggtt caagcaattc tcctgcctca gcctcctgag |
| 132181 | tagctgggat tacaggcgcc cgccaccatg cccaggctaa tttatttata tatatatatg |
| 132241 | tatatatatg tatgtgtgtg tgtgtgtgtg tatatatatg tgtatataca tatatgtgta |
| 132301 | tgtgtgtgtg tgtgtgtata tatatatata tatatttttt tttttttgag acggagtctc |
| 132361 | gctctgtcac ccaggatgga gtgcagtggc gccatctccg ctaactgcaa gctctgcctc |
| 132421 | ccaggttcac gccattctcc tgcctcagcc tcccgagtag ctgggactac aggtgcccac |
| 132481 | caccacgcct ggctaatttt ttgtattttt agtatagatg gggtttcacc gtgttagcca |
| 132541 | ggatggtctc gatctcctga cctcgtgatc tgcccgcctc aggctcccaa agtgctggga |
| 132601 | ttacaggcgt gagccaccgc gcccagccta atttttctat ttttagtaga gatgggtttc |
| 132661 | aacatgttgg ccaggctggt ctcgaattcc tgacctcaga taatccaccc gccacggcct |
| 132721 | cccaaagtgc tgggattaca ggcgtgagct accgcacccg gcctttatga tgcattttta |
| 132781 | ttattccttc agtagattgt gtgtttctcc ttgcatccag gcacttggtg ttatctaagt |
| 132841 | gtttatgtct tccatgtctg cactatgcta tatctgtagt ttgttttaaa tagtatctgt |
| 132901 | taaaacagcg accattatcc attgtagaca accagtcaca gtcctcatgg tgctgatgaa |
| 132961 | acacaaaagc cacaaagctt cttttcctca aataacctac ccgaggtgga gggctggctg |
| 133021 | gtttttgatg acacccattt tggtgaagac tcaggatgtg gggccacagg ttgcactgga |
| 133081 | cgagtctgtt tggctgccag tttcatcaga ctcacttgcc tcttgtgaaa tatttcctgg |
| 133141 | acatcatcca gcctctgcaa aactttctgg gctttggcct acagtaacaa aagcaaatca |
| 133201 | tgatgaacag gtctctctgt atacagcctg aggacacgaa gcattccctt tctcccacct |
| 133261 | tcccctactc cccccatccc tacctccctc aggtgacccc ctctgggcat cactatgcaa |
| 133321 | gtcgctgcag gtcctgccat gctccagaat tgggtatcta aggattagcc tctcctactt |
| 133381 | gagacccttg aggacaatga ctacatcctt tcactatggc gtctccagtg cctggaacac |
| 133441 | cctggcacag gagtttgaga ccagcctggt caacatggtg aaatcacatc tctactaaaa |
| 133501 | atacaaaaat tagccgggtg tggtggctgg cacctgtcat cccagctact tgagaagctg |
| 133561 | aggcaggaga atcgcttgaa cccaggaggc agaggttgca ctgagccgag atcatgccac |
| 133621 | tgcactccag cctgggcgac agagcgactc catctcagaa ataaacaaac aaacaaacaa |
| 133681 | ttagaataca atgtggcttg tgtcatgtta gacagagcaa agaattttgg cctgagtcca |
| 133741 | tggatggact tcagggcatt ccccagaatt acacaaacag ttagacaggc tggatataat |
| 133801 | ctgtttgttc ctccactgca tcttctcttc ctttctttgt gcccaggaag ctgattaaaa |
| 133861 | gaagctccta aaccagaggt gcagacaagg tgatgatgtg gctctcagag gaaggtgtgc |
| 133921 | tacctacaac cctgctggct tcatggaaga atatgcttct caaatgcaaa tccaatcaag |
| 133981 | ttatctccct gcttaaatcc aaactcgctc atgtggccca caaagcctac cttgcctgcc |
| 134041 | tctctctaac ctcacccaaa acacacttcc attgctctct aggttccagc accctgacct |
| 134101 | tttggtctgc actgtgccag gctccctcca gccctgaagc ctttgcacat gctgttcctc |
| 134161 | ctgtctggaa aggctctccc atcttgtcta ttcgttctcc aggctcaagt gactcttcct |
| 134221 | gagagccttc tctgacccca gtccaggtca agttcctctg tcacatgcct gaccctgcat |
| 134281 | ccctccttga cagcacttat atcagcttgt aacacatttt tgtgtgatta ttttgttaat |
| 134341 | gtcagcctct cccacaaact gtaagctcag tgagggatgg aagcatgcct atttttaatc |
| 134401 | atcatggtat cctcagcact cagcacagca catggtacat caggaatgct cataagcatt |
| 134461 | aatagagaaa tgactgattt gagtcagacg aagactcagt atgccaggac gttcaggccg |
| 134521 | agagtctaag aggccgccgg agggcttcgg aaccagtgag cccactcaat tcagcctgct |
| 134581 | cagtaccctg tgtgtacttt atcaggaaga aggtacagtc cctccctccc ctatccgttt |
| 134641 | cccaaccaca ggatcaaaat aacccggaag cattaccttt gcatcgaggg tgagcagcaa |
| 134701 | ctcaaactcg ttgtaaaact ccttggggct gagcaacggg tactccttga ctgtgcccag |
| 134761 | gaatgtcgca atgtcgttca aggcgatatc aaccccttct cgagactggc acttgtctac |
| 134821 | agcttgggaa gccaagaggt agattcctgc ctcacaccat tggctgacct tttggaaaga |
| 134881 | aacagtgccc tgtgcacttc gcctgaccac aactcagagc caccgtaatc ctcagcaggt |
| 134941 | ctgagcttgt aaggtgtcta caaggattcc cagacaatgg gaaattgtca tgggacggca |
| 135001 | tcgagtttaa ggggtctaac cactgtaaat tacactatga gggggacaaa cccaaaccag |
| 135061 | ctgatacctg tccacttctc agggaagtca ctcggtcagc aaaatgagtc tcttccatta |
| 135121 | acagtaattg ctacatctcc aggaggagac agctaaatat atatttaatt aaagacaggg |
| 135181 | tttcactctg tcacccaggc tggagtacag tagtgcaatt atggcttaat gcagcctgga |
| 135241 | gaggtctacc ccaggctcag gtgattcttc caccttagcc tcctgagtag ctgggactac |
| 135301 | agatgcatgc caccacacca ggctaatttt tccttccttc ctcccttcct tccttccttc |
| 135361 | cttccttcct ctttctttct ctctgtctct ctttctttct ttcctttttt tgagacaggt |
| 135421 | tttggccatg ttgccgaggc tgctctcgaa ctcctgggct caagcaatcc tcccacctcg |
| 135481 | gcctcccaaa gtgctgggat tacaggtgtg aggcactgca cccacccagc cttatagcta |
| 135541 | aatatctctt aggctggtct aaccatagag aaatgtaatc aatggctttt ggggtttacc |
| 135601 | tgctagtcca ctcttcttgc atccgaattg gttaccagaa cacagggtga ttaataagac |
| 135661 | attagacctg gcccttacat ttcctgggag aagggcatgt cccttttaac aaaaacacaa |
| 135721 | ctttcatctt tggattccca ggtgatccct atttgcattt actggcgttt gtctttcgta |
| 135781 | aggaagcaag aatgcaaagc tttgtaactc agcacagcga tactcaagag tttctggatg |
| 135841 | ctgggaggac attagatagg tgatgagttc taattattca atttttaaaa aagactcatt |
| 135901 | ttctttgctc ttttgaactc cactgagaca cttgaacaga aagacaggga gcccttatgc |
| 135961 | tacctgaaga gttatatgaa aagaagtttc cagtgccaat acctctgcat ttccatttac |
| 136021 | catgtgacct gaaaagcaat ataaagtaac tatacagaga aaaaagatca aaatgcaaca |
| 136081 | cgccaaagtg gttgtctttc taagtgggaa aatgataggc aacttaagtt tttgtcttat |
| 136141 | ttttttccta gattatttac agtgaatatg taccacattt agaataataa aaaatcataa |
| 136201 | agatatttca gaattaataa ttaccattat gtagggatgg gggatagtgg gatatgtacg |
| 136261 | tgcccatgag taaccttttt cttcctacat ttttggattt ccacaataat atgaactcat |
| 136321 | gctttgatat atgctgtgaa aactttctct ttacacatca tgcccagatc tgcaaaaaac |
| 136381 | attaggctgg gattttctgt agagttacta tggctacttg ctttctctct ctctctgtct |
| 136441 | ccctctctct ctctcaaaca cacatgcaca cacacacaca cacacacaca cacaccaggc |
| 136501 | ttataatctc tggagcaaaa atggagttgg gaaaacagca gctgatccca cagcgagctc |
| 136561 | agcacacaga gcaggctgca caccagcccc ttcggagcta cttgcttctg aaacaactat |
| 136621 | atttcattga cattaaaatc cctttagcaa tgggatgagg taagcgcctg ggtgggtgcc |
| 136681 | ccctgccatg caagaggaac agctgtgggg atggagcatg ctgactcatt gccgtcagcc |
| 136741 | acatgctgcc tgggaccagc tcacagggaa gactccagaa accgctgctc ctgctgactg |
| 136801 | ggaacatacc ctagaggtca cctccaaagg gcacccctct ccttgtgggc ccctggcatg |
| 136861 | cgcccttgct gtctatcctg tgaagccttg gtgtggaatc tggcaggcct gactttaatt |
| 136921 | ctggcttccc ctgtgtgcac catgtgtggt cttgggcagt catttacccc agggtttctt |
| 136981 | ggtgacatcc ctgcaccacc tgcaccagaa tcatttagac actttactaa aatactagtt |
| 137041 | cctgggcagt attccagacc tatcgacgct gagtcttcag ggcagtgcat aagggaatct |
| 137101 | gccatataac agctcactag gtgattctcg tgtgctacat tttgagaccc agcagagtat |
| 137161 | cagtttctgc ctctgcacaa tgaggccatt aataccttcc tcagaggttg tcttgaggat |
| 137221 | tataaataga tgacataatt atgttcctgg taggaatgca gtaggtgctc gatacatggt |
| 137281 | agggggtata aaatggttgt tattacttaa gcttctccaa aaagagtcaa aaccctttgc |
| 137341 | ctttatatga gggaagcact ctgcaaatat agtgcttggt tctctgtaca cctgaaaggg |
| 137401 | aatcaagaag tgttcccaca gggccaagcc aaaacacact cttaaagtcc ggctccacac |
| 137461 | agatagaaat gggcttcagt taactttaat gggatgtggt gggatggcca gagcactttt |
| 137521 | taccttcgtg ccaggtatac ttctacaccc caggatctgc acagttttta atgcctctcg |
| 137581 | aacatctttt cacataaagc actcaagcct ctcagcaatc acacaaggaa gggaggacga |
| 137641 | gtatgatgga agcttcttca cagaaaatct tgcagacatc atgttcttgg ctgtgttttg |
| 137701 | ctcaccttgt ccagctgtct atgaaactct aaggactttc ctaaaatgtc ccattttttc |
| 137761 | ttgtttccat tgatgaaatc gtcacagagg tgcctgagct ccacacaccg gggcctgatg |
| 137821 | gcatctgctg cataatggtg gctttggatg agctggtccc caaccagtgc cagcagcwgg |
| 137881 | gccttttcca ggggctcctg caaagtgaac acccacagcc aggcgtcaga agtcagagca |
| 137941 | tcatgaggct gagagctgcc tggtactgtg tgctccaagc ctggagggag gctgtggaga |
| 138001 | tgctaaccca ggatgaaggc tggagaacct gagaaagctc ggaatggttc caggcccagg |
| 138061 | aagttaagcc tggcccagag ttgtccacat gctgaacaac agcagtcatt cattcaaata |
| 138121 | cctgttgagt tctgggctag acatcgagga tatagtgtta acaagcccaa aattttcaga |
| 138181 | ttaatggaga attcagatat caactaaatt acacaaatcg ttaaattatt acaattgcaa |
| 138241 | taaactctag ggcagaaaag catggagtgc acataataag agattttgaa tacccctatt |
| 138301 | aactaaagga acaagaagag ggactgacac ctgtccttga ggagagtggg gggtgagctg |
| 138361 | aagttgaaac actggtgaaa attggatttg gacaactcaa aggtggtcat gacacttaag |
| 138421 | aaatatgctt gtctaggctg ggcgtggtgg ctcccacctg taatgccagc actttgggag |
| 138481 | gccgaggtgg gcggatcacc tgaggtcagg agttcaagac gcgcctgacc aacgtggaaa |
| 138541 | aaccccgtct ctactaaata caaaaattag atgggcgtgg tggtaggtgt ctgtaatccc |
| 138601 | aactactcag gaggctgagg caggagaatc gcttgaaccc gggaggcgga gattgcagtg |
| 138661 | aattgagatt gcgccactgc actccagcct gggtgacaga gcaagactcc atctcaaaaa |
| 138721 | aaaaaaaaaa aaaaaaaaga aatatgcttg tctaaaccct ttcccaaagc ctgtaagggc |
| 138781 | cctgtctaca ctgaccctgt ctgctcaatc acatttccta cctctttctg catgacactt |
| 138841 | cgctctaacc acacgtgcca agttttcctg tttgtttaag cattccttca ttcatccttc |
| 138901 | tcatattatt gagaacctgc tatttgccaa acactgtcat aggtgctggg agttcattgg |
| 138961 | taaagaaagc aggcaaaaac ccccacctca tgaaatgaag cttacatttt aatgggagac |
| 139021 | agaggcaata aacaagtgca tgtgtaggtc atatggtatc ttagacagtg atgagtatgt |
| 139081 | ggagaaaaat tagatggtgg ggagggcagg gagtgctgga gctagtattt taaatcggat |
| 139141 | ggtgagacag tctccctgag aaggagtact caagctatgc agaaaagact tcacatagaa |
| 139201 | agcctgagac taggccgggc tgtggtggct cacgcctgta atcctagcac tttgggaggc |
| 139261 | cgaggcaggc ggattgcctg agttcaggag ttggagacca gcctcggcaa cacagggaaa |
| 139321 | ccccatctct actaaaacaa aaaaaattag ccaggcatgg cagcatgcgc cgatagtccc |
| 139381 | agctactctg gaagctgagg caggagaatt gcttgaacct gggaagagga ggttgcagtg |
| 139441 | agccaagatc gtgccactgc actccagcct gggtgagagc taggctctgt ctccaaaaaa |
| 139501 | aaaaaaaaaa gagaaaagaa agcctgacac tacagcctta gaaagaaaga cccgatttta |
| 139561 | agattggccc tttgttggca tctaggaact tagatttttg ggaaggtttc ctccattccc |
| 139621 | tgatgtgaat ggttcatgat ccctgaactg tttgtgcaaa cagtatggtt tatggtgatt |
| 139681 | acttgctttt tcttctggga gtctgaaatc ttggtacttg ccagacagaa gctgctgaca |
| 139741 | tgaccagcca ccaataaaac ctctgggcat tgagtctcta atgagctccc ctggtagaca |
| 139801 | acatttcaca catgttgtca caacccacag acgggggcag gaagtatgtc ctgtgagact |
| 139861 | cccacaggag aggattctaa gaagcttgtg cctggtttcc tccagacttc acctcgcatg |
| 139921 | cctttctcct ttgctaattt tgcttagtac tttctcatct taatagatca taactctaat |
| 139981 | acagctacat gcagaatcct cctagtgaat cttcgaagaa atgtgtgcat ggtcttggga |
| 140041 | gcgctctgca tacaagctga gacctgaagt cagggacaag ccgtgcagta cttggctggg |
| 140101 | aggtgaaggc gtgtttgaca agtttgggta gcaggaagga ggccagtgtg gctgcagcag |
| 140161 | agggagcaag aaggaaatga gtggaagact aactagagga aagagggggt tccatagccg |
| 140221 | gggtgagagg aaagcccctg ggtttggagg gaggatctcg atcatgtttt tgaaaggacc |
| 140281 | actcccggta cagtgtccag aataagagga aatgcaggga gactcattaa ggggctgctg |
| 140341 | caggaatcca gccgagagat acggcttacg gctggtcacc aaactagctc ctgactcgac |
| 140401 | ccttctctgg attgttcttc cctgggtcat gcagagctga gttcctgggt cacctcctca |
| 140461 | cagcgcttcc ctggcgacta tatcttaagt cattctctac aacctattcg cccagtcacc |
| 140521 | taggccctct cccacacttt aattctttaa cagcattatc tctatctaaa aactattttc |
| 140581 | tttatttact ttaatgtaga attcctccct cctggctggg cgcggtggct cacaccagta |
| 140641 | atcccagcac tttgggaggc cgagacgggc agatcatgag gtcaggagat cgagaccatc |
| 140701 | ctggctaaca cggtgaaacc ccatctctac taaaaagaca aaaaattagc caggcatggt |
| 140761 | ggcacacacc tgtagtccca gctactcggg aggctgaggc aggagaatag cttgaacctg |
| 140821 | ggaggcggag gttgcagtaa gctgagattg tgccactgca ctccagcctg ggcaacagaa |
| 140881 | tgagactctg tctccaaaaa aaaaaaagaa ttcctccctc ctccactcac tcccaccaaa |
| 140941 | atgtaagttc catgagagca tttagtaggc atgcaataaa tatttgatca acaaaataaa |
| 141001 | tgaatgagcc aatgagctac atgatgcggt gacctgcttg gctggaactc tgtgaagcaa |
| 141061 | tcagcacaga tgactgccac attccaaaag gtctccagga gcagagagcc catgagcccc |
| 141121 | cttgccagcc acactaagca tccagtgcat cactgcactg ctttagctgt ttacagtaac |
| 141181 | acaaggagtc acggtaactc caggtgcgta catcttgccc ctctcttaac ttgcactggt |
| 141241 | ttatggtttc aacaatttcc aaaccccttt cctcttaccc tcccagtcat tcacgaagcc |
| 141301 | ccgcagggac cgtcgccttc ttaatgcctt tcaaattctg accccatctc tgccacctcc |
| 141361 | ttgtcactgt ctgctcaggc ctcatggctt ctcatgggga caactggtct ccctgctttt |
| 141421 | cttcaggctc tccttcaaat catgctccag actgctgaca aaggagcaat tccaacatat |
| 141481 | cttattgtgt ctacatcact cccaagcttc aagccctttc ctgggcgccc agggtcttca |
| 141541 | ggagcaagct taaactcttg agcaatgcac acggggaatt tcattatcta gctcatacct |
| 141601 | gggtgtgcct gacttaccaa gtcctcttcc acttcctcaa ctccattctc tgctgcaacc |
| 141661 | atactaatta tccacttatt gttccccaaa tattcaggca gcttcaggcc tccatccctt |
| 141721 | ctccacctgc tgtctgccta gaaagcccat ccttccctac tccccctcac ccatactcct |
| 141781 | ctggggccaa ctcgatctaa atcatatttc agacttcacc ttctccagaa aactttctct |
| 141841 | gtgcttctct tgatcagagc acctgcttca ttatgttgtg accacttatc tgctgcctgc |
| 141901 | gctatgccac agacacctat ggtccagaac cagctcttat tcaactgaaa acagtgcatg |
| 141961 | ctgcatctta ggtacttaca aataaatgct gcacaaaaca aagaggagta ctggtataga |
| 142021 | aacaagtgac acaaatcaag tagtggctag ttaaaaggac tgaggcaaaa atacccagaa |
| 142081 | cacttgttct aaacagcaaa ggcccaggtg cactggttac cacccctaca agatgactgg |
| 142141 | aatactcatt ccctgggcag tgattttttt tttttttttt tttttgagat ggaatcttgc |
| 142201 | tctgtagcta ggctggagtg cagtggcttg atctaggctc actgaaaatt ctgcctccgg |
| 142261 | ggttcaagcg attctcctcc ctcagcctcc cgagtagctg ggactatagg cgcccaccac |
| 142321 | tatgcctggc taattttttt tttttttaat ttttagcaga gatggggttt caccatgttg |
| 142381 | cccaggctgg tctcgaactc ctgagctcag gcaattcacc cgcctcggcc tcccaaagtg |
| 142441 | ctaggattac aggcgtgagc caccgcgcct ggtctgggca gtgatttttg actctttggt |
| 142501 | tcactgctgt gtcctcagaa cttaaacagt acctggcatg gtaggagatg aataaatatt |
| 142561 | tgcagaatag atgtcgaatg aatgacagac ctttcctagc actgctcagc taccgcagac |
| 142621 | tgaacctagc acatgttcca tagcccagaa tttatccctc tacctctgta atgttaataa |
| 142681 | caacagctaa catctactga gagcttaaca gatgccaggc actgttctga atgctttaca |
| 142741 | tttgcttatt tccttaactc ttcacaacaa accctttgat acaggtactg ttttaccaaa |
| 142801 | gggaagtgga gcacaatggc ccaaggtgac ccaggtagga aagagaagag ccaggcttca |
| 142861 | aacccaggca gtcttgctcc aggatctggc tcagaccaac atacacagag aaaaatggct |
| 142921 | gcacttgttc tgcctaaact tagagacctc atttggttgc tcgaacttgt ctgactttag |
| 142981 | tcaaatatag aaacatgaga cttggcatga aagtctctgt tgacactttt ttttttttta |
| 143041 | agtccagctg cctaatttat taagaacagg gcagaatttt ctgggtccag ggaaattcac |
| 143101 | cacaggatac ttccaaatca ccctgtggtg atcagagcct gcccttccct cagcatacag |
| 143161 | tatttcaccc ttcagaattt tccagaagac atttgagatg ttagagggaa gatgtgtgta |
| 143221 | acaggtccaa atgggtctag ggagaaacgc ccataaaggc aactgaggag gccgggcgca |
| 143281 | gtgggtcacg cctgtaatcc cagcactttg ggaagccaag gccggcggat cacaaggtca |
| 143341 | ggagttagag accagcctga ccaacatggt gaaaccccat ctctactaaa aatacaaaaa |
| 143401 | ttagccgggt gtggtggagg gtacctgtag tcccagctac ttgggaggct gagacagaag |
| 143461 | aatcgcttga acccaggagg cggaggttgc agtgagctga gattgtgcca ctgcactcca |
| 143521 | gcctgggcgg caagagtgaa actccgtctc aaaaaaaagg gcaactaagg agcaacccgg |
| 143581 | attcagccac cacatggctg gaaggtcact attacagaat tgaaaggaca acaatagttt |
| 143641 | ctgaccactg actactgacc accaaccata ttgcaagctg accacatgaa gtccctacca |
| 143701 | cacatctaac tcatttaatc ttcacaaggt ctctgtgagg caggaattat tacctctgct |
| 143761 | ttacagatga gggggaccat attcagaaag gctacgaaac gtgactaggg tcatacagct |
| 143821 | ggtacttgtt agagccaaga tttgaatcca tccctatccg acttccaaag cctgtgttct |
| 143881 | tgtcaccttc cagcccacct ggcctctaca catgtgctat gaaatcctcc tggtgcctgc |
| 143941 | aaagaaattt atggtgaaac ctccaaattg aagcttattt ttttagttta tatacctccc |
| 144001 | actcaggata aatagtcatc aaaatctcca aagtaaaaaa gaaagagtga gggaaaaaat |
| 144061 | ggctaaaatc ttctgatcac aagcccatct gggatctttc agatgctgaa aattccagaa |
| 144121 | ggctggatcg tcaagtttca gcttagaagt tgcaatcaga aatctttatt gaaataagtt |
| 144181 | tatttccccc cgaggactct gctggtataa cagatgagag atgagagtgc ctactaggga |
| 144241 | agaaagtggc gagaaagcga gtgatgctac tctatccggg aatctagaac aaacccacag |
| 144301 | aggatccaca gggatggagt gtgtgtatgg ggaggtgggc cagatgaaac aagaaaggca |
| 144361 | gaaagctaat ggttgttgaa attgaatgat ggacacttgg agttcattgt gccagtctat |
| 144421 | ctgcttctct gtgtgtttga aaatttcaca catgtacaca aagccatgac cctccaacaa |
| 144481 | gatgacatcc ttagatgtta atggcctggg ttccatgcct tcttagacac agcaccctta |
| 144541 | tggggagagg tcaaggctga aatcaggtgg ttcctccacc ctgtcctggt ctgttccctg |
| 144601 | ctggcccctc agcaagggtc tcagtacggc ccacagtcaa aaccctgcag caagttctgc |
| 144661 | ttttatagaa gaccttctgt aagtggttga aactactgtt tttcagaatc aggaaggagc |
| 144721 | attcaaaaga ctgctaaacc ctcaaacgag aacaagataa agaacaggct gaatcagcat |
| 144781 | gcgacaggag cctggttagt gagttgactg aggttacttg tgagaatgat gtcactgtgt |
| 144841 | ggaataagga agctggcaac accccagagc cagcgtgtgg ctgtgctggg agggagctgg |
| 144901 | ccccttagtg ccgagtaaag gacaaactcc aggcagatat ttgtcagatt gtggccagac |
| 144961 | acagtcttgt tcctttaaca ctttaaagga caaatggaaa gattagccaa gccctaatca |
| 145021 | tctgaagaag ccatgtgtct gagaagtctg agggaagata tattgaaaac gatctcagtt |
| 145081 | acgatgagac agctttgttt gatttacgca aaggcagaga cacaggtgta ctacatcctt |
| 145141 | caggaagcaa gtttagaaat aagatctgca agtcatagga tgtgacaggt ctgcaaatcc |
| 145201 | tgcaagtcac aggtcatcca cagtgggcta gtctagctgc ttcaagaatg ttgcccacga |
| 145261 | aaggtgggtc atgggttcac agagcattcc tgtgttccat aaccacaggt ttcatcccta |
| 145321 | actctggtca gcaacttttg aagtatatac caaggaccac aagttcaagg aagtgggcag |
| 145381 | aagaaagaat gtgatgcagt catgcagatc catcgccact attcaagaca cagcccagag |
| 145441 | cgccttccaa gggctttctg ttccactgag aataaaattc aaatcctcac aaaagatctc |
| 145501 | agggtgcaac aggactacct ggcctctgct catctctgtt cactcatttc ctcctggccc |
| 145561 | acctgctcta gcaatgttcc ttgaactcac caagctcatt tccactttag gacctttgca |
| 145621 | cctgcagctc gctcttgctt tgatactatt ccctaagtct ttgcatgatt acacagatca |
| 145681 | aacagatctc agaccaaagg tcgtcttctc aggcctttcc agaccattct agctaaagta |
| 145741 | gcatccccag tcactttcta ttatatcttg ttttcttttt tatatcactt gttactatct |
| 145801 | gatgttatta tttatttgat tgctttttag tgaactgtct ctctccagta gaatgtaagc |
| 145861 | tccatgaggg cagattctgg actgtctggc tcaccgctgc acccacggaa cctaggataa |
| 145921 | ggaatacctc ttgatgaata aataaccctc tggataggtc agtagctact ttcatatata |
| 145981 | tatgaaggca ttttttttga tgaggcactt tttcttaata tatgaataag caaaaaaata |
| 146041 | tagtatctat attcagtaaa aaattatttt gaaaaaacta aaatgatata aggttggggc |
| 146101 | tgtcctggag aatctaaggt atctagtcat catatatatg aaattcaact tttccattgt |
| 146161 | ttgataaatg ttcctgatga ctcatgtgtc tttagcacta gtttatctgt caaatggtca |
| 146221 | ctggtaagag aaagagcgct cgctgtgaag ggatgggatt cctgaatggg gatctggata |
| 146281 | cacagctggg tttgtgtgtt catgcatgtg cacaagcacc tgcagctcag aatctacaac |
| 146341 | caacagctct tagagtcagt gtggccttgg tatactttgt tttgtatcct ttaggggtgt |
| 146401 | gtgtctccaa ctttaacgta caagtcaatc acctggggat cttcttaaaa tgcagattcc |
| 146461 | aattccaggg tggggtctga gattttatat ttctaattag cgcttactca ggtgacatct |
| 146521 | gtgctgctgg ccagaggctc acactttggc aaatctctag tactgcaaaa ttgccgtctg |
| 146581 | agaaacatgc ctccagatta gctaagaaag gaagcgctaa ccacagggga cttggtcccc |
| 146641 | aagacccaaa aatatagaga acctaatttc aaacaagcag aaaggaactt ctctggaaag |
| 146701 | agacgactca gctttcttca tttgtaataa accaaatctg taaacactta aaatggaatc |
| 146761 | atttctaaat gctgacttgt gtgcggatct gggacactgt ttaattccag tatgggactg |
| 146821 | taaaatacat attcttttac aagtttcaca tttacaactc ttgggacatg agaaaggaga |
| 146881 | aagctttgcc aattctgtgt agcaatggaa ctaacatgga ccaatttccc ccatctaatt |
| 146941 | gtcttgctcc aggtataaaa ctagttgttt ttttttttct ggtttctcct ttgaaagttt |
| 147001 | cccatcctta ctaccaaccc cacaaccccc agccccctag tccaggctcc agggaggagc |
| 147061 | tcaagctgat gggtgagcct ctgatataat tgttctggtg agtgatgagc ctgttgtgac |
| 147121 | cttctcccac ttggctgctg accacgtgag agggcagggc tggtccagag tcctcacccg |
| 147181 | acctgagcgt ccaactgcaa ctgtatgttc cggcagcttt aataggactc tgtgctgccc |
| 147241 | gtattattca tagcagttgt taatgtgcct ctctccttgg gatcctgggg tgactgaagg |
| 147301 | gcaggactgg gcattcctgt gtcctttagc aaccttccaa tagcaatgac tggacatgat |
| 147361 | ccatcattgt ccacagcaac ctccctccca gtacacaaaa atgtcagaaa gcaaacctgg |
| 147421 | cttttttcct ccagtttttt gtgttcctta agaaKctgct ccacgtgcat cacgctgtct |
| 147481 | ccaatgcctg taaactctgc ttgctcttcc agcaaattat ccagggcaag cttagcctac |
| 147541 | aagaaacatt agaacagtcc agaagtaaac caacactcac tcacctgaag gtataggttt |
| 147601 | gttatgcttc aagaaaactc aaaattccaa aaaggattca agacttgtat atatctatat |
| 147661 | atgttaaaaa tttttttttt gcagctcact aactttcaaa |

Following is a KIAA0861 genomic nucleotide sequence that includes the KIAA0861 SNP “KIAA0861-AA” (SEQ ID NO: 4951).

GTGCCTTTCTGAACCCTGTGACCCAGCAGCCTCCATCAACTCGTCCTACC
TGCCATGCACAGCTCCTCTGTGCCCCTGTACCTGAGCTCATGCTATTCCC
TCTGCCAGGATGCCCTTCTCCTTCTCCACCAGGAGAAGAACACTTGCCAG
TAAGACCCAGTTCTAATGTCACCCCTTCCTGACGGTATCAGGAAGAGTC
A[G/C]TGATGGTGTTTTATGCTCCCAGAGAATTTGCCACATTGTGTTG
TGATTATTTTTCCACATCTGTCTCCCCCACTGGAATGAGAGCCTCACTC
ATCTTCATACCTCCCTGGTCTCTACCTGGTGCCAGAACCATCCTCAGGG
CAGGGGAATGCTCAGGAAATAGATATTGAATAAAATAAGTGTATCCATC
CATCCATCCA

Following is a NUMA1 region genomic nucleotide sequence (SEQ ID NO: 4).

| >11: 71420751-71612100 | |
|---|---|
| 1 | cctgccatgg ggcctggcac atagtagcct gctctgaatt tttgttgaat gaatgaatct |
| 61 | cagcaaattt ataattttcc aataaacaat acttccttct tgagtcacaa agcagccact |
| 121 | gcctgtataa ttctttttat tttattcaaa ttcgtttctc atttgatcct ccccaccagc |
| 181 | ttgtgaggta atttaatatc tgYactcaac agaaaaagag attttctcat tcattcaaat |
| 241 | aagcacctac tgtgtgccag gccctgtgct gagtgctggg gatacagagc tggataagac |
| 301 | atggtccctg ccctcaagga gtactctgtt taatggagga gatggacaag aaagctcctg |
| 361 | ttgcagtgca gttggtaagt gctatgacag aggtttgtgt aggtccaaga gagcttgact |
| 421 | ggccagcctc aaagctgtga ccacctgatc caaagtcttt caaattgtag attgctcttc |
| 481 | ttcctattct tcttatgttc tactttcctg caagcctttc tctggccttc tggagtttca |
| 541 | ttgtataagg taaaataggt gatccataag ttcctctctg gctttggtag tttactttaa |
| 601 | aggccactgt ggaagataca ggatgtggag ccagatgaat gtggattggg acccctagcc |
| 661 | ctgccacttg ttcactgtat gaccttacct ttctgagcct tgaagtcttc atccctaaaa |
| 721 | atgggacaat aattgctacc tcacataggg tgataagatg atatgaagaa aaagtgcaat |
| 781 | agaacaatgt aagagtcacc aaggacacca caaacagtgc caacttctgg ctaaaataat |
| 841 | tgaaaatatg tctgtaatac agaggatttt tataatagta actactagct ttttttttga |
| 901 | gatggagtct cgctctgtcg cccaggctgg agtgcagtgg cacgatctcg gctcactcgt |
| 961 | gcaagctccg cctcctgggt tcacaccatt ttcctgcctc agcctcccaa gtagctggga |
| 1021 | ctacaggcac ccgccaccac acctggctaa ttttttgtac ttttttagta gagacagggt |
| 1081 | ttccccgtgt tagccaggat gtctcgatct cctgacctca tgatctgcct gcctcaacct |
| 1141 | cccaaagtgc tgggattaca ggcatgagcc accgtgcctg gccagtaact actagctatt |
| 1201 | tatttattta tttagagaca gggtctcact ctgtcRccca ggctggagtg cagttcactg |
| 1261 | caacctctgc ctcccaggct taggcgattc tcctggctta gcctcccaag tagctgggac |
| 1321 | tacaggtgtg tgccgccaca cccggctaat tttttttttt tttttttttt ttttgtctat |
| 1381 | ttttagtaga gacagggttt caccatgttg gccaggctga cctcaagtga tctgcccgcc |
| 1441 | tcgggctccc aaagtgctgg aattacaagt gtgagccacc acgcccagcc ctgtaactaa |
| 1501 | catttgatag tgctttacaa tttcaaagca ctttttaaaa atagagatga ggtcgcactc |
| 1561 | tgtcgcccag gctagagtgc agtgccatga tcacaactca ctgcagcctc atcctcctag |
| 1621 | gctcaaccaa ttttcccatc tcagtctctg tagtagctgg gactacaggc atgctccacc |
| 1681 | ttgtctggct attttttaat ttttattttt agtagagaca aggtcttact gagtttccta |
| 1741 | ggctggtctc gaactcctga gctcaaaSaa tcctcccact tcagcctccc aaagtgttgg |
| 1801 | gattccaggt gtgagccatt gtgcctgccc tcaaagcact tttgtgaaca tccttattta |
| 1861 | tacatgtagc atttcatgaa catgtattat ataccaggaa atgggctctg tacaacccag |
| 1921 | taaaaaaggt agcagggttt tcctgtttta caaagaaaat cacctctctt gtctcaaaga |
| 1981 | ggtgatttga cttatctgag gttatccagc tactaaaaca tcacttaatc catattgagt |
| 2041 | tttttgacct aaaatttcac atccttttca ccgtgtctct ctcttttgtg atcagtgcaa |
| 2101 | Wccctggaga atagagctgt gtgtgttctt taagacaaca ctagaaggaa gatatttggg |
| 2161 | acctttccct agcctgagaa accatcactg cttgcctggc cacagcaagg aggcatgggc |
| 2221 | agggaaggac cttgactttc ctccctaggg ttcagcaggg ttggtagtaa cctagtcagc |
| 2281 | tctcaggaac cacacaggct tgtgctggtt attcagacag attactaaat tgttcattag |
| 2341 | ctcccatcac agatagaata gtggctcctg atgacaggga cctactcttg tagcatttaa |
| 2401 | aaggctctgg tttggccaag caccagtggc ttatgcctgt aatcccagca ctttgggagg |
| 2461 | ctgaggtggg aggatccctt gagcctagga gttctagacc agtctgggca acattgcaag |
| 2521 | accccatctc tagccaaaaa aaaacaaaaa aaaaggccct ggttccttca gggaaccatc |
| 2581 | tttcttgctg agtctagtag ctgggatcca ccaattaggt tgtacactgt cttcattggc |
| 2641 | tcctggggca cactactcct ccttcaggag catgaacaac cccattctac ttcaccacca |
| 2701 | ccacacactt tcatccaact caaaggtact tcttaagtgc ctctctccct tctccctcag |
| 2761 | aagaaaattc atttcagaag caaattcatt acacatcatt tctttcattt ttggcctgtg |
| 2821 | gataggagca aagctgtatt cagactaacc gggcccaggt acaaacaacc tgtagctcat |
| 2881 | tctacctctg attcatctct tgaattctgt ttctgaacaa ctagccttcc ctgcctgtta |
| 2941 | atcttctgag catagttgtg tttaatactt tttacttcct catttgaggt gacatagctg |
| 3001 | tctaaggtca gctgtgctcc ttctttgagg cagaaactgc ctttttagga atcttttcct |
| 3061 | tggtctgaaa ccatgaaaat acaaagcata gctgggtgtg gtggctcacg cctataatcc |
| 3121 | aaacactttg ggaggctgag gcgagYggat catgtggtca ggaattcgag accagcctgg |
| 3181 | ccaatatggt gaaaccccgt ctctactaaa aaaaatataa aaattagccg ggtgtggtgg |
| 3241 | tgggcacctg tggtcccagc tactcaggag gctgaggcag aagaatcgct tgaactcagg |
| 3301 | aggtggaggt tgcagtgagc tgagattgtg ccactgcact ccagcttggg tgacagagcg |
| 3361 | agactctcat ctcaaaaaaa aaaaaaaaaa aaagaaaaaa aatacaaagc cctagttctt |
| 3421 | gagtatatgg aggcatttct gcagttaagg gagtgagcag ctttggatag taactgaagt |
| 3481 | tgctcagtaa gtcctgagca aagaccctgg tgtccccatt gatgtgagaa taaaggattc |
| 3541 | tggtgatcag gaccattaag gccagaatct ctgtgtcagc tgcactaacc ttctctcctt |
| 3601 | tccccaggtt ggtttataag tggttcctgc taatctataa aatcagctat gccactggca |
| 3661 | ttgttggcta catggctgtc atgtttaccc tctttggtct taacttatta ttcaagtgag |
| 3721 | taccctttgt ttttgttttt gctggagttt ggggaggagt gactgttgag atgtacctct |
| 3781 | tgttgcaagt cgtggttatt aacaggaggg acgtaatatc caggatagga gacttgggtg |
| 3841 | ctgtgggggg atgggagaag acaggctctc catgatttgt tctggcagaa agcaaggaat |
| 3901 | gtctgaatat atacaaagtc agagtcctaa atcatatttt ccaaggcttt atattttagt |
| 3961 | ggaaaagtct agtaaccaaa gtaagcataa taattatcat aacctgctat ccataattct |
| 4021 | atcaccttaa cagagctgtt tttctcgtca gtgtgtactg ttcatttgca gcatccactc |
| 4081 | tgcatgaatg gccttacgtt caactaggcc aggggttggc aaactgtgcc ctctgtgcca |
| 4141 | aatcagccca caacctgtta ttataaaatg aagttttact ggaacatagt catgcccatt |
| 4201 | catttatata ttgcctgtgg ctgtttttcc actaaaacag cagagttgaa tagttgtgat |
| 4261 | aaagaccata tgggctacaa aacctaaaat atttaccatc tggcccttta taggaaaagt |
| 4321 | ttgctgactc ttgggctaga ccgtctacct acatgaccgt ggttggctac agctagcaac |
| 4381 | aaattgactt agaagaatac aaaataaata atagagcaga gtggtgtctg caagtcttca |
| 4441 | cagtagactc agagcttagg tgtgagggaa tgagaYgaca cagatgggtg tggaagccac |
| 4501 | ctggcctagg agccccattc ccccaggaac acatccagtt tcaagaatgt cgcagtccag |
| 4561 | atgtgttgta ctaatcacag gtcataagca gtgttgcctg taatatagct gacattcgac |
| 4621 | aagatttcta aggaaacagt tctggaaagt taacctgtgg atgttaagtc ctgcttcatt |
| 4681 | gttgggcact taggttgttt tcaatttagt attatcccag ataacgctgc tctgttgctt |
| 4741 | cctaagggtg cattcctagg gatggaattt tggctgttgc ttcagttgtc ataagccttc |
| 4801 | caaaagatct ttattagctt acaggtcctc ccaccccgaa ttgagtcaga tgccaagaaa |
| 4861 | tcctgagctc agagactgga gcatgcagtg agtcaatgag gcagatgagg gtggaaataa |
| 4921 | tgctttcctg gatgaaatac tggtggctgt gggctgtgga tcaagaggag ccctaagcct |
| 4981 | acgacagtgc actgaagcta gagatctagg attcaaattt aagataatga atgggatgat |
| 5041 | cactggcctc ttccctgagc ctagttatat tgtaggcagg agtctctgaa aggagtttat |
| 5101 | tttaggacaa gtaaaaataa atcctggcca ggtgtggtgg ctctcacttg taatcccagc |
| 5161 | tatttggaag gctgaggcag gaggactgct tgaggccagg agttcaagac caagctgggc |
| 5221 | aacatagtga gactctcaac tcttaaaaaa aaaaaaaaag aaagaaagaa agaaaattag |
| 5281 | ctgggtgtag cagtgtgcac ctgtatcccc agctactcag gaggctgagg tgggagaatt |
| 5341 | gcttgagccc agaagttcaa ggctgcagtg agctatgatt gtgccactgc actccagctc |
| 5401 | aggtgacaaa gggagacccc catctcgaaa aaaataaaaa taaataaatc ctattttata |
| 5461 | tagtgagagt gcactcatga aattctttct tctaggggta gtacaggctg aaggaataaa |
| 5521 | caggctcctg aagggtttag aggaattcat cactgtagag tcaaacacat cttgtcaaag |
| 5581 | aaaacagata tctcctggta actttctaac ctgtatggtt aaaaaataaa ataaaaatca |
| 5641 | agattctgca ttatgggatt ctgcattatg gttttttgtt tgtttgtttg tttgtttgtt |
| 5701 | tgttttgaga tggagtcttg ctttgtcgcc taggctagag tgcagtggct cactgcaacc |
| 5761 | tccacctccc gagtagtggg gtcaaatgat tctcctgcct cagcctcctg aatagctggg |
| 5821 | actacaggca tgagccacca cacccggcta atttttatat ttttagtaag gatggggttt |
| 5881 | caccatgctg gcgaggctgg tttcgaactc ctgacctcag gtgatccacc tgccttggcc |
| 5941 | tcccataagt gttaggatta caggtgtgag ccaccacgcc tggcctctgc attaagtttt |
| 6001 | aaagataatt cttctataat tacagtttct ttttacttgc ctgaactcta caatgaggaa |
| 6061 | aacagaattg agaatcttga tcatttttgt gctactgatc aaattaagtg gtgtaagatc |
| 6121 | aatRatttct agtgagttaa atattcaggg ttcataagca aatgcagctt tgtatataaa |
| 6181 | tataatttat aactaagcac atacatacgg ttctttgtac aaatatttta acagataatt |
| 6241 | ttctataaca aagatttata gactcaaggt tgcaaattat cggtccttgg aattattaaa |
| 6301 | acaaaccttc tgatgttata ggcaagatct ttatccttct tgttaaatag acttggacca |
| 6361 | aaatttaact ttgatgatat gatacagatt atttatattt gtagtttata attttatagt |
| 6421 | aactgctata tattaataaa tatttactaa ttttatcttt ttttcttaaa cctgtgtttg |
| 6481 | acaagctgtt tctgtaaagg tccaaatagt aaatatttta ggtcttatgg gacaataggc |
| 6541 | caaatctagg atatcatgta ggtacttata taacaagaga aaaaactcct accctgtccc |
| 6601 | accctctttg cctggggaag gggccaggtc tcaggtagtg ggcatgcttt gtgctctgtt |
| 6661 | gtcataagct gagacaatct cagaaagagg ggcatgtggc caccggagta attttcagYc |
| 6721 | tctgcaccag tgacacccag attctggcat gccctttcct ccttatggat ggctcagctg |
| 6781 | tttcaacttg agtggctagc tggaagagag acagctcagc aaggcagctc acttagtgtc |
| 6841 | cagaagttac caacagtgga gtccccacta tgtaggtccc gatgagcata ggtcctgggt |
| 6901 | agctgcaagt cattgtcacc agttRgggga ggcaaggact aagagcagag tgaggaaaag |
| 6961 | gggtaggagg ggatactgta gcttggtctt cacagcctac agtgctagct agcctcctca |
| 7021 | agaggtgttc atgggccata taaaaataga cagcagactS gatttggtca tagtttgccc |
| 7081 | RcacttgctY tagcctttgg gccttctcgc tttgggccct gccttgtgtg tcccagagta |
| 7141 | gtaatccata gaagatatgt ctcagggtgg gtctttctag ctctaatgct tcccttgcag |
| 7201 | gatcaaacca gaagatgcca tggactttgg catctccctt ctcttctatg gcctctacta |
| 7261 | tggagttctg gaacgggact ttgcagaaat gtgtgcagac tacatggcat ctaccatagg |
| 7321 | ggtaagttca tccgggttct taaatactgc ttcttgacac ctccaaattg ctcaagttga |
| 7381 | tgtcacttgt ttaatggaag agaaaggcac tagggtgaga taagccacat aaaatccttc |
| 7441 | aagatgtgca tatgcatgga cattccttcc ttaaagtttg gaaagcaaca cagccataaa |
| 7501 | aaaagataaa accatgtgcg ttgcagcaac atgccagctg gaggccttta tcataagcca |
| 7561 | gttaatgcag gaacagaaaa ccaaatactg catgttctta cttataagtg ggaactaaac |
| 7621 | attggatatt catggacata aagatggcaa cagtaggcac tggggactac aagaggagga |
| 7681 | gggagtgggg acaacRgttg aaaaactatc tgttaggtaa ctgtgctcat tacatgggca |
| 7741 | acaggatcat tcgcacccca gacctcagta tcacgcagta tgcccatgta acaaacctgc |
| 7801 | acatgtaccc tctgaatcta aaataaaagt tgaaattatt aaaaaaaaaa ttagccagat |
| 7861 | ctcagatgta ccctataatt tttctcttta gggccattat aggaaagagc cactttatcc |
| 7921 | agcaggaagt taacattcat tcagtagata ctttttaatg cctgctatgt gtctagacac |
| 7981 | atagctatRt gccagaatta caaaagtgta caaaaataaa catggttttt gcccagataa |
| 8041 | actttactct taggtgggag agatggctat tacagtaaca aacacagata aaaataaaat |
| 8101 | tacaacttta ataagcatta tgtaggaaag gttcatgatg ctgtaaacac atgtagtagt |
| 8161 | gtcatttgac ctagtcttgg ggctcaaaga tggcttcctt gaagaagtga ctatggagct |
| 8221 | gacttgaaca atgaatagca gttaagtgga tgaagtgaga accaggcaga ggaaagagca |
| 8281 | ttcataagag tcctgtcacg ggagaaagtg tgggtgtttt tgaggaacta agaccattgc |
| 8341 | ggttgtatgg ccaagaagtg cctggagaag tagtgactag actagaaaaa agcctgtggg |
| 8401 | ccaaattaaa gattttgatt tttatgctaa aaatcctggg aggggccggg cacagtggct |
| 8461 | cactttggga ggctgaggca ggcagattgc ttgagcccag gagttcaaga ccagcctgag |
| 8521 | caccatagtg agaccccatc tctacaaaaa aatgagacaa aaaaaaaaaa aacctagccg |
| 8581 | ggcgtggtgg cacatgcctg tagtcccagc tactttggag accaaggtgg gaagtcactt |
| 8641 | gagccttgca gtgagcttgt tcatgccacc aaactccagc ctgggcaaca gagcaagacg |
| 8701 | ttgtctctaa ataagtaaaa attcacaaaa tgtataataa tcatgcaaag gcacaacaat |
| 8761 | aaatgatggg gcacaaaagt gcttagtcca tattggtaat tgtttttgaa ctgcacagtg |
| 8821 | gtaggagttg ctccttacaa tttttacttt gtgaactctt attccttgat ttaacccaca |
| 8881 | gccgctacaa caacagccac tgtcacttac tgctttacca aaaattgggg aagctttcat |
| 8941 | acttgctttg ttaatcttta actgtatgtg taaagctaca catttcattt caacatttaa |
| 9001 | tattaaaagt gttttgagtc tttacttaag agacaatttg gtgtctctta aatagaggtt |
| 9061 | tagtgatgtc tttgtgacag aaatatgaca caggaactga actcttgttt ctatcaatta |
| 9121 | gcctgtggta aaattggttt tgttatatgt cgtttcattt aaagtcacag tttccaagaa |
| 9181 | actgtcagtg aagtgaggac atactgtatt tgagaagtat tttggactta gatatagttt |
| 9241 | ggttctgaag actgaaggga aaaatacaaa ggaaatatag gtttcaaagc tgagatttct |
| 9301 | gattcggcaa ctgcttggat ggtagtacct ttcactgaga taggaaatgc tgcaagagca |
| 9361 | gcaggtggac atcacggaga gatgcttaaa tatgggtaaa ttaacaRcca atctgagaaa |
| 9421 | cgcaagtaaa tatttctatt ttttttttca gaatggaaat atatctttat tgttttgctg |
| 9481 | tttataaaag tagtatgttc tctttgtaat tatttaaaca acacagaagt gctccctgtc |
| 9541 | tcagtgtcat cccccaagac cccaccccaa gaaggaatat ttccatcacc ttgggtaact |
| 9601 | ttaatctagt accgtttaag gccagggact ttgtccctca gtatcatggg aatattctaa |
| 9661 | ttttttctct actggatcaa attgaaaaca gtacattgKa tcactttatt gacagtttat |
| 9721 | taggttcctt tttgaacaac tgtcttgtgc tttaccttca tttccctctt acctatttgt |
| 9781 | gtagacattg gatcttcctt taaccagttt actccccaga agagatttct agaccatgtc |
| 9841 | accattttga ccattttgaa gtatacaatt catgcattaa ttacatttgc agtgttctgc |
| 9901 | aacawtttca ctgtctaatt ccaaaatttt tcttcatccc aaacagaaac tctgcccatt |
| 9961 | aagcagtaac tcattacccc ttcYcacagc cctggtttac ctctaatata ctttccatct |
| 10021 | ctgtgaacat gcctattgta gatatttcat gtaaatggaa tcatacaata tttgtccctt |
| 10081 | tggctttttt cacttagcgt agtgttttca gagttcatcc atgtatagaa tgtatcattc |
| 10141 | ctttatatgg ctgaatagta ttccattgtg tgtgacaccg catttggttt attcattcat |
| 10201 | ctgtggatag gcacttgagt tgtttccact ttttggctac tgtgagtaac gctgtgagca |
| 10261 | ctggcgttta agtgtgttta gtccctagtt tcatatcttt tgggtttata ccgagaatgg |
| 10321 | aattgcttgg tcatttggta attttatgtt taactttttg aggacatgcc aaactctttg |
| 10381 | ctttcttaat tatgataatg cttaagtgaa tatctgtatt catgaagctt ctttgacact |
| 10441 | tcctcttttt tttttttttt ttgagatgga gtctcggtct gttgcccagg ctggagtgca |
| 10501 | gtggtgcgat ctcggctcac tgcaagctct gcctcccggg ttcacgccat tctcctgcct |
| 10561 | cagcctcctg agtagctggg actacaggcg ccccccacca tgcctggcta attttttgta |
| 10621 | tttttagtag agacggggtt tcactgtgtt agttaggatg ctctcgatct cctgacctcg |
| 10681 | tgatccgccc tccttggcct cccaaagtgc tggggttaca ggcgtcagcc accgcgccca |
| 10741 | gcctcctctt atttctttaa attccttgaa gtgaaattaa tgggtcaaaa ggtgcaacaa |
| 10801 | tttgagatac catgctaaat cctatctgaa aggcttgaac cccttttgct gctgccacaa |
| 10861 | atgtctgaga atgcccttgt caccaaacca acatggcact taatatttca ctgttttaaa |
| 10921 | tgttggagaa tattgttgtc catttctggt ggaaccatag tctggcgaca gaatggcttg |
| 10981 | cctgacacag cttttgatca ccagccccaa gtccttctcc ctccccttcc ccttatcctt |
| 11041 | tggtgctgat ccagcccagt ttataggttc tgttacatct aacttcccaa ctgtctctag |
| 11101 | tctggtcact tctccccatc tgcccagcaa agatgaggtc gatttgactt gtaaaagatt |
| 11161 | accttttcta aacagtttaa aaaaaaaaag atgtcctcta actctgggcc tcccgctgcc |
| 11221 | actgtgcctc ttctctagaa ggcctgaggt ctctgtggag cttcctactt tggctgcttc |
| 11281 | tcacatcttt tcctatcttt ccttcctgct attctccttc agttctacag cgagtcgggc |
| 11341 | atgcctacca aacatctttc agacagtgtg tgtgctgtgt gtgggcagca gatctttgtg |
| 11401 | gacgtcagtg aagaggggat cattgagaac acgtataggc tgtcctgcaa tcatgtgtat |
| 11461 | cctgcctcga gctcctgggc cacatctctc ctgcaatctg cacactgtag tgagagagag |
| 11521 | aaagagggtg gtcatgaccc taggcatgct gtccagaccc ttgagtgtag gagagccaca |
| 11581 | agagggccac cttccctacc ctttcccaca gatcagagcc actcttaggt aaacagtggt |
| 11641 | tacaacctgg ctgggtctga gaggaacctg aggctgcagc tagtgaggct gtgctagcag |
| 11701 | acctgagatc tgaccagtca caggggaaga ggaccctcta atgggatccc tcagtttagc |
| 11761 | atcatcctct gttctgaata acacttgtaa tatttatcat gtatttaccc agctcccatt |
| 11821 | ctctgcctat gttgggaaag ccatgtattg tctccatttt gccccataac agtcttgtga |
| 11881 | gatagggatc attatcctta cttcacatgt gaggaaacag gttcagacac agggacttgc |
| 11941 | ccaacattac agagctgata aagagcgaag gcaggatttg aacctttcaa agactgtctg |
| 12001 | gggctggagt gccgccctgg ctctcaccat ttcccttgac cagcggtgct cagcttccac |
| 12061 | gagttctgca tccgtggctg gtgcatcgtg ggaaagaagc aaacgtgtcc ctactgcaaa |
| 12121 | gagaaggtag acctcaagag gatgttcagc aatccgtatc ctttattggg gtcgttgttg |
| 12181 | ggagtgggct gtgggaagaa agtactggcc agtgtgacct gcatttgggt cctcctgggg |
| 12241 | ccctcacttc tgcccccaac cagaacatgt caaaccatgg tgggaagctg agctgctagt |
| 12301 | cctgccccca tgatacaacc ccagtttccc tattctagga ttattgccta ttctcagcct |
| 12361 | ccttgcagcc attgtcacgg atagatagac ttgtgtcagc ctcaggctgt gttatcttac |
| 12421 | actggcagtt ccttcttaga gaaggtgtta caggaggccc tccctgcatg ttagtcatgg |
| 12481 | ggaactagga tttatctcta tacctctctc agggctgccc tgcctccagt accagtcccc |
| 12541 | tctatctgcc tcccaccttc tctgcattca gctctttacc ccaacagaca ggctgtgagg |
| 12601 | aggaagagga gggggagggc aaagttggga gggctgcctg agtaggaagt gaggctgggc |
| 12661 | ccttccagaa agcagcctcc attcctctag gctgtggagc agctggaaag tggggcagac |
| 12721 | ccagggagcc ccaccaccca tgctagatca ctcctggcag ctcttgcaca gagtctcttt |
| 12781 | tctttaacac actgacagct gggagaggcc tcacgtcatg tatgggcaac tgctggactg |
| 12841 | gcttcgatac ttggtagcct ggcagcctgt catcattggt gtagtccaag gcatcaacta |
| 12901 | catcctgggc ctggaatagt gatgaagagc atcagtggaa aacccacccc acacgccatg |
| 12961 | gacctcaggg cactctcctc cctgcccaca aagacctcct gggtgggaaa gactcaaagg |
| 13021 | ggYgcttggg ccactcagga cccctcYggc tgtgtcggac tggggaggga tatgatggag |
| 13081 | agccagccag tggggctgtc agcagtgggg ggctttttaa aagaaaacta ttttgatgaa |
| 13141 | tatatttaaa aaaccttttt ttattgtgga gcataggaat tgcccccctc caggcttcac |
| 13201 | cctccctgcc taagcagggt gggggcagag ccatgacatt tttggtttaa aggagccttc |
| 13261 | tcatctctgg ccgagaacac tgctgggctc ccaggtagct gaaggcctca gcccacccac |
| 13321 | tcccttcttc cctgtgtggg gctcaagcct ggtgcactta gtatagaaga gctaacgtac |
| 13381 | tcaggcttgt gccacgggtg agcactgagg ccccaggtgt cctccctccc ccactgtctg |
| 13441 | ggacacagga caggtgttgc gtctgactct gtctccccct gtccaagagc tataggctgg |
| 13501 | ttgaggcaag tggaacctta tcagtgactt ggccaacaag ggaagccttg gggcctcaga |
| 13561 | attcattgaa Ratttgtttt gggacaactg gaagaatcag caccatggat actcgcccgt |
| 13621 | gggcagggct gtggtcagcc aagaatagag gtcatatagt tgggccatgg ggcaattgtt |
| 13681 | ggcagccatg actgacggcc ccttgtgcaa gctcattctt cagtggatcc ttcagtggag |
| 13741 | tggtgacacg tgccagcggt cactctgcca catcacttcc ttcttgaaag cctaagagtg |
| 13801 | cgtatgcgtg tgtgtgtgtg tgtgtgtgtg tacgtgaggc agaagaatat gacagatgtc |
| 13861 | agcttctccc tgtgtcagat gatatgtcct tgaagctggc aggggattcc cagtccaggg |
| 13921 | aatatcccta catgattttt gtcattcatg ggtcctccct gggcctgaca acgggagtgg |
| 13981 | tgggtgggtg agaacaggtt ctgagagggg gcttctagga aggccccagc cctagtccta |
| 14041 | ggcgacttga ataaagtggg aggtcaaaca tgggccttcg gcattKggtt tctagggaat |
| 14101 | atggctcagt agggcctgcc ccttcaaggg cagcaagaac gaagcaggat gttgtcactc |
| 14161 | ccgtctgaat aaagctccat gagctgggtt ggaaagctga acatttatct tgctttcttc |
| 14221 | ttcccacctc tttccctggg gtccccaggg gtgtcttgcc tttgttaacc cccatcggag |
| 14281 | acctgagggt ctgggggtgg ttgggcctgc ctttctgctg ttcccagctg ttccccwgct |
| 14341 | gacatcccta ggagtggtgg aatgccaggc tcctgtcctg tggcctgggg gcctggggca |
| 14401 | tcattgggcc tgccttcaga ctgaggttgc tctatgttct cactgcctga ggacagtgtc |
| 14461 | cYgccaccac ctgccttcct gaggaccaca ctcaggctcg gcttctcttc cccagctRtg |
| 14521 | gcttcccatg gtaccctgct gcttatgcca cctgtggggc tgttcccctc ctcagagagg |
| 14581 | ggaatggggc tggtcagagg acctagggat cacccttcct tttacttttt ccccccaccc |
| 14641 | ccctgtcagg ctcaggtgag aagccgaggc caggctgtcc aaagctcttt tcatRtgtaa |
| 14701 | cagttctgag tcccttgacc tccccccttg gcctgactct ggtctcggtt ccactaagac |
| 14761 | agagtcctgt catctttccc taccatcccc cactggactg tctctctcag aaaccccagg |
| 14821 | cagccttgcY tcctgaccta actcctggag atgctggtgt tcttgggagg aatttctcct |
| 14881 | gcatcttaca tgctagaagc ccctggggca ggaggtaggg ggaggaattg agaggaggaa |
| 14941 | aaacatgaac tagacaccta gagaagaagg atgtgacttg tagtatccta tgtctaaatt |
| 15001 | aggaatatga atctggtttt tctacaagaa gtttgagatc acagctgact gtgttcctga |
| 15061 | tgcatccacc aaacccagtt ccatctgtgg gcctccctgg ctctgtcacc agccgttgca |
| 15121 | ccctcccaat cacaggagtc acaaacctca gacatgcagc tcctgtccac acttaatata |
| 15181 | tgcatgcatt ggatcaccca gccctggtct ttctgcctcc atggataact gcatgaccct |
| 15241 | gagagaaaac ctccttagat ttagcatcct aggttcctca cMcgcctcac cctgaatcct |
| 15301 | ggccctcccg cagccccagc gccatttgtc ccatcagtga caagattcat attctgatgt |
| 15361 | agactctgtt gccagagcca gtgttgagcc agtccgcctc ttccccggga agtgcctgcc |
| 15421 | cttccctcct gttagRgttg gctctcgagc ttgtgtgcca gttcctgggt tggccgtgag |
| 15481 | agttctacag acaaggagga agtgctctcg gtgtatttcc tgtggtgggt tcacacgcag |
| 15541 | ctagacacag ctaacttgag tcttggagct cctagaggga agcttctgga aaggaaggct |
| 15601 | cttcaggacc tcttaggagc caggtaggag tctgggacta ctagtgaacc tagacctgtg |
| 15661 | gctctggcca gaggggctag gatgagagac agagggtgtg atggtgggtg ctgggagatg |
| 15721 | tagccgacct tggggctggt ggctRggKga gtgggtagcc tgggaaaggc caggatgtgg |
| 15781 | acggactggt atggcaYtga gcctgaagtg gtccaacttg gggttcccca gtgcctagga |
| 15841 | aagttgtccc cttgaatgtc agtgtgaagg tgaaggagga agcagatgcc tgttcatatg |
| 15901 | gaaacaaaga cctggctgtg aagaggggag gcggacacca aagtcctgac acttgggcgg |
| 15961 | gacagaattg atctgtgaga gactcatcta gttcataccc taggtgaccc tgggggtggc |
| 16021 | atgggggtag attagMgatc ccagtctggt atcctctgga gagtaggagt cccaggagct |
| 16081 | gaaggtttct ggccactgaa ctttggctaa agcagaggtg tcacagctgc tcaagattcc |
| 16141 | ctggttaaaa agtgaaagtg aaatagaggg tcggggcagt gctttcccag aaggattgct |
| 16201 | cggcatcctg cccttcccag aagcagctct ggtgctgaag agagcactgc ctccctgtgt |
| 16261 | gactgggtga gtccatattc tctctttggg tctcaatttt gccttcccta atgaaggggt |
| 16321 | aagattggac taggtaagca tcttacaacc atttgtggtc atgagagctg gggtggggaa |
| 16381 | ggattgtcac ttgacccccc cagctctgtt tctaagtgct gaaagagctc caggctatgc |
| 16441 | tacgggagga gaagccagct actgaggaaa agccagctac tgagaaaaag cgggagtggt |
| 16501 | ttaccattct cctcccccac ctttcaccag agaagaggac gttgtcacag ataaagagcc |
| 16561 | aggctcacca gctcctgacg catgcatcat gaccatgaga cacaactgga caccaggtag |
| 16621 | gccttggggc tacgcatggg caggcRgggt agggtgaggt ctatgaacag aatggagcaa |
| 16681 | tgggctaacc cggagccttc actccaaggc aaRccaccca gcgcacctgg tgctgttgct |
| 16741 | ttaagaacct gggcagatat tgtagctctg gctccagtct aaagcttctc tgtactctgt |
| 16801 | tcaataaagg gctaaggggt gggtgctgag gggtccctct tcccgctctg attccctgRc |
| 16861 | tagaacccag acatctctgg gctggagtta catccttacc cgggcagccc actNtgtctc |
| 16921 | cagagccgct gacctgtaac tgtcctttcc tcagacctca gccctttgtg ggtcctgctc |
| 16981 | ctgtgtgccc acgtcgtcac tctcctggtc agagccacac ctgtctcgca gaccaccaca |
| 17041 | gctgccactg cctcagttag aagcacaaag gacccctgcc cctcccagcc cccagtgttc |
| 17101 | ccagcagcta agcagtgtcc agcattggaa gtgacctggc cagaggtgga agtgccactg |
| 17161 | agtaagaagc acagtggtgg agggtgggct atgggcacag aggttcccag ggtcgggttg |
| 17221 | actcctgagc gccagtcccc ttctgcccat gtaccaccag ctgagccagc tgggctgagc |
| 17281 | acgcaccatt ctccctcccc aacccagtgt catgggtgca ggcttggcgc agctcccaag |
| 17341 | atgctcccta tcaaatagga cagagaactc aagacataag taatggtcac aggacctccc |
| 17401 | agagccttgg ttgcagtgga cYccaaggcc agcccctcca cccagagcct gctggcctct |
| 17461 | ggccatctca gaggagcagc agccatccag cactgcctYt gtcacctggg ctcccaagtc |
| 17521 | accgaggctg ggcactagaa aaggtcatcc tgaggagaca ggttcagaag aggattcatc |
| 17581 | acgtgaacca aggaccattc ctcacattcc ccgtgtttag ggctagggcc tctcggagac |
| 17641 | aactgSactt ctgtaacgga cgttcccacc taggtggtgt Rcagagcagt tctctaggtt |
| 17701 | ccagatgcat ggggactggg gggagctggc agRgagggca cagcagagca gggtagggga |
| 17761 | agggcctgct cttctgaaga gctaactgct gcctgtgtcc ctagatggaa cgctgagctt |
| 17821 | atcctgtgtg gcctgcagcc Rcttccccaa cttcagcatc ctctactggc tgggcaatgg |
| 17881 | ttccttcatt gagcacctcc caggccgact gtgggagggg agcaccaggt gagggtcgca |
| 17941 | gcagccaggt gggtgggaag gaRgccttct gcggccttct catgaccttt ccttcccttc |
| 18001 | cgctccagcc Rggaacgtgg gagcacaggt acgcagctgt gcaaggcctt ggtgctggag |
| 18061 | cagctgaccc ctgccctgca cagcaccaac ttctcctgtg tgctcgtgga ccctgaacag |
| 18121 | gttgtccagc gtcacgtcgt cctggcccag ctctgggtga ggagcccaag gagaggcctc |
| 18181 | caggaacagg aggagctctg cttccatatg tggggaggaa agggtgggct ctgccagwgc |
| 18241 | agcctgtgaa ctaatgccca gcattcctca aggtcagcca gacaaaaagg aacttaggtc |
| 18301 | ttgggcagag gaggtgtagc ctggggcaaa gtgatgagat gtccctcctt tccttggcct |
| 18361 | gatccttgtc tgccttcact tccctaggct gggctgaggg caaccttgcc ccccacccaa |
| 18421 | gaagccctgc cctccagcca cagcagtcca cagcagcagg gttaagactc agcacagggc |
| 18481 | cagcagcagc acaaccttga ccagagcttg ggtcctacct gtctacctgg agtgaacagt |
| 18541 | ccctgactgc ctgtaggctg cgtggatgMg caacacaccc cctccttctc tgctttgggt |
| 18601 | cccttctctc accaaattca aactccattc ccacctacct agaaaatcac agcctcctta |
| 18661 | taatgcctcc tYctcctgcc attctctctc cacctatcca ttagccttcc taacRtccta |
| 18721 | ctcctcacac tgctctactg ctcagaaacc accaagactg ttgatgcctt agccttgcac |
| 18781 | tccagggccc tacctgcatt tcccacatga ctttctggaa gcctcccaac tattcttgct |
| 18841 | tttcccagac agctcccact cccatgtctc tgctcattta gtcccgtctt cctcaccgcc |
| 18901 | ccagcagggg aacgctcaag cctggtYgaa atgctgcctc ttcagtgaag tcatcctctt |
| 18961 | tcagctctgg ccgcattctg cagacttcct atcttcgtgc tgtatgtttt ttttttcccc |
| 19021 | cttcactcta atggactgtt ccagggaagg gatgggggca gcagctgctt cggatccaca |
| 19081 | ctgtatctgt gtcatcccca catgggtcct cataaaggat tattcaatgg aggcatcctg |
| 19141 | acatctgttc atttaggctt cagttccact cccaggaact ttgcctgtcc cacgagggag |
| 19201 | tatgggagag agggactgcc acacagaagc tgaagacaac acctgcttca ggggaacaca |
| 19261 | ggcgcNtgaa aaagaaaaga gagaacagcY cataatgctc cccgggagca gaggccacta |
| 19321 | atggagagtg ggaagagcct ggaaagatgt ggcctcagga aaagggatga gagaaaggag |
| 19381 | gtggtatgga agactcagca ggaacaaggt aggcttcaaa gagcctatat tcctcttttt |
| 19441 | cccacaccga tcaagKcaac tcagtactca cgggagaaaa atagacttta tttacaagta |
| 19501 | ataacattta Raaaagatcc atccccggcc cttaaaaacc ttcccatcac tccaaatccc |
| 19561 | accccagtgc aagtYtgggg aaggtagggt Rtgagctgct gctgaaggct gtcccccaac |
| 19621 | cccactcctg agacaYaggg cccatccgtc ctgggaaaga gcatcctctg gcaggtgctc |
| 19681 | ccaccaggtc agacccagtc ctggacttca agagtgaggg cccctgctgg gcccagccac |
| 19741 | caggacagca ggaaccaggg cctactcctY ttatggtccc ttctagatcc agaggctaag |
| 19801 | aggaagactg gccaggccca aggacccagc catcaaaacc agcctcaaat ctggttgtga |
| 19861 | tggagaagtg actttgcttt aagaaaaaag gaggcaaggt agggagagcg cccacactgt |
| 19921 | ccatgctcca ggccccctgg gccagctccg agaaggcgcc agtgaaggac cagggaccag |
| 19981 | gccagggtgc gggcaggcat cRctgtctct aggggtttgg ctactgttgg cctgggagct |
| 20041 | gagagaaggc actgagaggg acagtaggcg gaggaccagg tgaSgKcagc atcggggaca |
| 20101 | caggtggggc cactcactgg tactggccct ttagtgcttt gcctgaaaga gacacagtca |
| 20161 | catggccaga tgagaacttg cgatactagc ctgcacccac tggctgggaa gatctcttcc |
| 20221 | tgctcccacg cccctgtctg gatccYctcc ctYgtgagcc ccagggttat cagttgctgg |
| 20281 | ctgtgcctga gcagctctgR gtgctctcca tgagaatggg gccatctgtc ttctctcctt |
| 20341 | ggagaggagc taccaggaca gggacacctc ttaccccaca ccctccagca gcctggcgtg |
| 20401 | gccccatctt ggatgctact tggtggggcg gtctgggggg tgcccatgct ctcatcgggt |
| 20461 | ttccctcccc catcctgcca gtgcctctac cttgcccttg gctcgagggg tggcaccaat |
| 20521 | ggcggcagca gtggcggcRc tggctgtggt ggtggcaatg cgcggagaac ggcgggttcc |
| 20581 | actgcgagtg ttgggggaag ccttggacag ggccttcttt gaggctcccc gccgcagaag |
| 20641 | gctgttccct agcttcttgg gtgtgttgag gatgctgaag gYcatcgact ggcgccggtc |
| 20701 | agcctgcaag gaagggctgt cagaccggga gacccaatgc tgccttccca ggccagcgtg |
| 20761 | ctgtgccacg ctStaYcagc aaggtcccgc cagggcgtcg cttcatcccc cttcagcccc |
| 20821 | agcctcacct gtttagtaga agctggagct gctttcttct gggcctcagt agtgctctgt |
| 20881 | ttgcgccctt catgtcggtc tcgKggagtc atggggcRtg ggaaacagct ggtggccttc |
| 20941 | ttagactatg gagaagagga cagttaggca gacagtaRca agaggagtca catctgaagc |
| 21001 | caggtgtctt gtcctctcag agctgagtgg accttgtaag tcaacgtgca acctgctccc |
| 21061 | cttcccaact ctgggccaga tccttccctt cccaacagtt cccatccatg ggtcaggccc |
| 21121 | ttggagagag ggaaagagag ggggaagtga gggaaggaga gagaaggctc cctttagtcc |
| 21181 | ttggtgagct gggcctgacc tgagcacagt gctggagtaa cacccaggag ccaccgcgcc |
| 21241 | tacctcagga gttccagggc cctggtggKg ctctagggag acccgtttgc gctgctgccg |
| 21301 | ggtggtgatg ccagtgccct cggctatctg gattggctgc atgctggctc ggcRcagggt |
| 21361 | ctcttggggg tctccagttt tcatctcctc atctgtgatg gtgcccaggc tcagggaagg |
| 21421 | ctgcatgggt ggaagaggtg gtcagtggac catagctgta tggagatgga ggaggacctg |
| 21481 | gggctgtYcc agaactctac actcgcccga cacttatggt cgggaccctt cctgcctcac |
| 21541 | gaggtagaaa gacacaaggc ctcctttcct gttctgcttt ctacctaagc cctgggcaaa |
| 21601 | tggccacaag cagtgcagtc ctgaccagat tcctctctga gctcctgcct accccMaggg |
| 21661 | acttcacccc tgagtgccct cccagctgtc tgttccacct ggaaacatga gaaggtcacc |
| 21721 | ctccccctcc tcggccagtc agtgatccag ggccctagtg ctcaggctag atcagcaggt |
| 21781 | gggattccaa ggRagggcag ggatgggagg ccctgcacag tgaccccagg cctcaccctg |
| 21841 | gactccaggg gatagcaggt cttcagatgt ggggggcaca ctcgattgcg ctgctgcagc |
| 21901 | tctgcaatgc ggttccagtc atccagctgc tcaggctcat cctggcaagt gcccatgtag |
| 21961 | aagctgttcc ttcctgtgga aggcagggaa gtgggaacaa atgagcctgg agtcggcagg |
| 22021 | tcacctcctg gccctggcat cttgccagcc tttgctgcca cctaccccat aaacttgaag |
| 22081 | cccRgcacac cagtctgatt cagtgccgca ggtgcaggag tacggcacac agactatttc |
| 22141 | tatcctaggg gcttgctcac caccttctcc ctggagaggg cagaagaggt cacacgcaga |
| 22201 | gactgctact acatcttaYt cacctgccaa ggcttggtgg ccaacaccca gaggaacaaa |
| 22261 | ttaaggaccg ggaattaatt cccaggggct ccctggtgcc caaaggacaa gagcttccaa |
| 22321 | gaagagtctg gccagcctgg cctttccagc agcccatcac cgcctgagaa gggcatggag |
| 22381 | gactccccac agctaagtgt cacaattgtg ctgggaatcc cgggccctta actctggcta |
| 22441 | agagtgcccc caacacagcc agcccctaga tgggcaggta aggaaggccc tgaggctgca |
| 22501 | ggaaggaggg gcaggtggag ctggatggta gcaaggaggc cagccttgga tttttaaaaa |
| 22561 | gctttcctct tttccctgtg ccacgatcca ccttccagtc taattttggg gtatagtaag |
| 22621 | tccctgtagt cccctcacct ggaggggccc cactggacac cccggcctgg gaacgacgag |
| 22681 | cagaactgcg agtggtgggg cggtagccag gcaagctgag cagggctgag ttgccataat |
| 22741 | cgggagaacc caggcgagct agagactgag tagaggaggt ggctcgcagg ctagcctggg |
| 22801 | aagcaggagc agacMgcgtg ctgtagaacg atgagttggc gctgtctggc tcttccaYat |
| 22861 | ctagcttctg gaagacagag tgaatctgtt gcagYgtaca gtccctggca ctgtacagaa |
| 22921 | gcttcccatt cccttccgaa gccctcagat cccacggcac atccatgtat tcccaactgc |
| 22981 | tttgcaaagg tccttaaagt gtgtgtctgc aagaaatggg ccttgtcgac agaagccctc |
| 23041 | acaaggtggt gctgatgttg tcaagactct tctacgcatt tttttcatgg agtctattca |
| 23101 | taatgctttg aggtagggaa tgcagagtgt ttatcggccc attttggaga tgaagtgcaa |
| 23161 | agaaataaag tgactagccc caaatcacac tgctaggaag tatcagagct ggggctaggc |
| 23221 | cccatgtctc ctgactagtc aggctcatcc cacagcctct gctgtccctc agtccaaact |
| 23281 | tccagggccc ttaccatgtt ccagaacttc cccaacttct tggtagcagg gggcacccta |
| 23341 | aacacacagg tcccccctgc tgtaccaggg gcccctctcc cctcctccca aacctcccct |
| 23401 | tcaagatgtg gaaacaaagg caagggcctg Yagcctgtca ggcagtccac tgggcagcaa |
| 23461 | caatgcctct cagctgcatg gggcatgctg ggaggcacag gatgggctgc agcttcgcca |
| 23521 | cgttctctcc cttcaccctg cacaggctca gtgctacgca tggagagaat gctagcctta |
| 23581 | gtcaggaggc agggatctaa tcctagccct gcctttttct tcagaagtgc ccttaaccaa |
| 23641 | gtcactgccc tttttaagac ctctcagctt tcccactgta acatggactg gctgctcatc |
| 23701 | cctccctgct cctgactgag tgcccagtgc aaagatgccc ttgagaggaa gtgggaattg |
| 23761 | ctgccctgca gcccctgtcc cagcagcctg accttggtca tggtgatgtt gatgatctgc |
| 23821 | gtggtgcgcc gacgagcgga gcgggtctta cgacccgagt ccaggaagac gtctcccagg |
| 23881 | gagtccaggc tgctctccag gggggcctga ctccgagcag ggatgggagt gaagtagaga |
| 23941 | ctctccaggg attctacctt ggggggcRgg cgctgggaga taggtgaggc tggttctcca |
| 24001 | gggacgctgg tgccgtctgg ctgggtacga ggcagcttgc tatggaaagg aaacctgctg |
| 24061 | aggtacagtc cttcacgcta gaaggcattt tgtccagccc tttttgaaag tgaccatttc |
| 24121 | ccatccttac tacaagcaga tgaggtcaag cagcccatct ctctgatttt tcagaggtac |
| 24181 | caaggaaagc acttgacctg ggccttcctt tcagtccact cccctggctt ctcctaatag |
| 24241 | ctcctcatag tggcaatgcc cagacccaga tgtcccatcc ccaccagatg cccatggctg |
| 24301 | cttcacagca aacacgtccc aatctaagcc cacttctctg ttcttcctcc tgcatctccc |
| 24361 | atttcagtga ataggaacgc cccccactca ggcccccaag ccatgaacgt gggcataatc |
| 24421 | caatgtcgtc ctgcttggcg agcccaatca ccaagcttcc tgaacagcWM ctaaccacac |
| 24481 | cccactccat gctcatgtcc tttccctggt ctgcttcatc tcctctcacc tagactcatc |
| 24541 | atggcactga cagaggaaga tgagtggtga caagccaaga actcaggcca ggctgcccag |
| 24601 | ggtcaaaccc cagctctacc actggggaac cttgggcaag tgactgcccc tctatgcccc |
| 24661 | agattcatct gttaaaggag acagaataaa cctacctcag agggttgttg ggggacaaat |
| 24721 | caggtataat tgtcatgagt gctttatgtc tagcacaaaa taagtaaatg taagtgttta |
| 24781 | ataaaaacat ttttccagtc ttaacttcta tactagctat tagaataatc tttttaagat |
| 24841 | gcaaattaac caYgtactct tcagtttcaa attcttccaa ggttcctttt tttttctcct |
| 24901 | aaacctttca attacagcca ccaaggtttc ctcaatgcct ttaggatcaa gtcttaatag |
| 24961 | Ycggtctcca atgggccttc ctggcctcac atctcagctt agtcaggctc tttcacagcc |
| 25021 | tctgttcaca ctgctcttct cgtcaacctg gcacacacac ctatggatgc tttcaagact |
| 25081 | cagcaggagc gatagcttcc acgaagcctt tcttgaccac ttgcttttta ttccaactgt |
| 25141 | gccctaaata gatatctggt ataatacccg tttttcKtta ttcttctcta agaataaatt |
| 25201 | tagatcacat cttatttatc tcactaggat acagcctgat aaacagcagg cactccataa |
| 25261 | acagacatag ggaggaaggc tgcctcctga cctggtgata ctgagtgggg tcccctcctc |
| 25321 | gcagctcaga tccaggctgt caatactcaa gtccagctgg ggcttagcct ggggctcacg |
| 25381 | gctctttaaa gcatcagttg ccacctggaa tttgcccagg tctcgaagct gctggtctgc |
| 25441 | atgggcaacc tgagaaggag agggccaggg ggagagtgaa gaaaagccta aggcagcagt |
| 25501 | ggcctaggag gagagggcac aggggtccag tggaatagga gtgaggtgag tctgccagcc |
| 25561 | aaattcttac ctgtgcctcc aggctgcgca cctgggcagt aaggtggcgg caggtctgtt |
| 25621 | cagcctcctt ggtcttcagc ccagcctgct gtagctcatg gcccagccgt tcagcttcag |
| 25681 | ctcgcagctc tttgttttcc ttctgcagct gctccaggct ccgcagctgc tcctgcagct |
| 25741 | cttggttctg ctgcttcttg gcatcataat gtgttttggc tttctccatc tgtgggcaga |
| 25801 | gagggtgggt gggtggggta gaggttgaag ggcaggagga aggtggcatg ggctggggct |
| 25861 | gggctcctta cctgcagctt atagtgctca gctgcctgct ccttctggct caactgggct |
| 25921 | tgcagttcat tcagctgggc ctggaggcgc tgggcctcct gctggctctc gcctccctga |
| 25981 | gcctggacag cctgagaaga aaggccactc aggaaagctg gtctggccgc tctacccaag |
| 26041 | tccgcttatc tgcaccagcc actggggctc cacaggtgct tcagactcct gcactttctt |
| 26101 | aggaagcgga agaaaaccac accaccccaa atactctctg cccgactaca gtctactaaa |
| 26161 | cctgaagccc acaYgccctt cagtccacat ccgtagtggc ctttccctga ccagctcctg |
| 26221 | ccctatcttt gccttctttg agaccagaca caaagtgtca caccctcttt gcatgcacaa |
| 26281 | tctcccgtag gaggaaggga accaatcctc agggaacacc caggtgcaga gccNtaSWSt |
| 26341 | aaggcacttg gcatgtgagc tctcttagga caggggaact tgtttctcct caaagccact |
| 26401 | ctgagaagtc acttcaccct cttttcagag gaggaaactg agttcagaga tttaagcaaa |
| 26461 | ttgaccagag taacagtgac taaatggcag agtaagattt ggtccaccta actccaaaag |
| 26521 | cccatggggg cttatttatg atgcccacac tgcctcctgt ccagaccata actccttaag |
| 26581 | agcagggcct gattctctgt ccatctgcaa ggccaacaag tgctttgctg tagatagaag |
| 26641 | catcccccct ttgcctctcc taacatagga caggccccac aaggagtaga ataaacattt |
| 26701 | gtttattgca atcaagacat gctgtggcac catggtatga aggtgagcag gagaaatgaa |
| 26761 | gctctgagct cccaacaact cctaggagcc accagggctt tagtgagtcc caatgccact |
| 26821 | ggtgcccaat tcctttgccc ctgactgaag gctgtgaagc ctagaactgc aaaaagcaag |
| 26881 | tattaaacca aagaaggtga cacagttgaa agcattaaga atgttattaa actgcactgt |
| 26941 | aatattcaac actggtgaca ctctYgttct ttccctgccc agactatcta gacttacatt |
| 27001 | gtccaaaaga actttatgca atggtggaat gttctgtatc tgcgctgtcc aatacagcag |
| 27061 | ccactaacca catgtagcta ctgaacaggg gcacagtgta gctagtgtga ttgaagaact |
| 27121 | ggatttactt taataaattt caataaccac acgtgactag tagctgctga agtggacagt |
| 27181 | gacagttgag agatgggaac caccctggtt gtctctcgag agctcccaga agacacatgc |
| 27241 | aggggcttct aagccacagg cttcctagga gggctacStg gaatgcaggg tgcccctctt |
| 27301 | ctgccccgac accccccacg gccccagagc ttagagaata gcttccctcc cttgtccaga |
| 27361 | ggccagaccc attcccccag ctgccccacc ttcagcttct gctgctgcac cttgctggct |
| 27421 | tggtcagagt cagccagttt cttactcagt tcttccacct ggggagggaa gaggaggaca |
| 27481 | gaagactcag gaggacttcc cctggatgtt catcctggat gtccacccat catggggttt |
| 27541 | cccagaccag gatccccagt gttcctagaa taaactcagg ccctttctct ggcctccaat |
| 27601 | tacggaacag cctgctgccc aggctgcttg gcagggcctg aatgaggcaa cctaacacat |
| 27661 | agatgttgcc agggccaact gttcttgcgt ctaccctgag cctgcagaga cagaggcctg |
| 27721 | cccaaccccc gtcacctcca aaatcaggag aattgactct ggggagccga cacctcacca |
| 27781 | ccaccttgct gccgccacac tctgcagccg caaccttccc aggacctagt ctgtaattct |
| 27841 | agtagacata ctcaatgaat tcccatgcct ccctcccaga aactgccaaa gcccaagccc |
| 27901 | acagatgctc agacctcttc ccaacatcca caggccgtta gggctggtgc tagcagcatg |
| 27961 | gctgctgtgg ttagcattgg ggaacagatt gctggaagac aagagcaggg gcagcacatg |
| 28021 | tactccaaag cccaccccac tYctacaaag aggaagagca cagggagtag gggaaaagag |
| 28081 | agaacataac agccccatgc acccttggct gctccaagca ttctgggagt tcaagcatga |
| 28141 | ccaggcagca caggggagag gcaagggctc cattgctaac tgtttgctat tcactttgat |
| 28201 | tgtcccccac tatcacaggc tccttagagg agggcccaat ttttcagcag ccctggggtg |
| 28261 | gctgggtagg agagtgatgg agaaagggtg ctctatagcc tcgatgtctc agaaacacca |
| 28321 | gctctatagc ctcgatgtct caggcaacac agagatgatt cccatgagaa agaggcagcc |
| 28381 | agaccatgtg acccccactc ccccgccccc atctggtata gacccctcct gctccttcac |
| 28441 | cctcggtctt cctgctacag acatctctat gctgtgctcg gggttagagc cccagaaagt |
| 28501 | gatgggcaag tgtcctaagM ccacaatcct ccMtaacctg tgggggctgc ctcgcatatc |
| 28561 | accttcaagg cttccataaa gctgtactct gggaaaaagg gacaccatgt taaacaaggc |
| 28621 | cgacacggta attctgctgt tcctcttagt cgggtatcct taactagaca cagtacttgg |
| 28681 | gaagttctcg aatcccaagg attgtgtctg tcattggttc agggtgggaa agatgaagcc |
| 28741 | agtgctaccc cttgtccctg ataRgcaaat gagagaaggt gcagaagggt ggagaggccg |
| 28801 | acctgggatg ggtgggatga ggcctgagag agcccagcac accacactcc tgggcagcct |
| 28861 | gggcctggat tccatggctt gaccctgccc ccttcacaaa cagttcacaa caggagctgg |
| 28921 | cggaggcaag ctgcagccgg gctcatgcac caagactggt caccgtttaa gcagccagca |
| 28981 | tttagcgagg acctcaggca ttacctgctt agtttgctct ctctgaaata cctctagctg |
| 29041 | ctccacctgt acatggggga ggagcaacag agacagagtg agatgaggcc aaagaagcac |
| 29101 | cagccctgct gccaggctga cctcactgag gctctgatac tttcccctaa ggagctagat |
| 29161 | tgacagtaag tttaaaaatg ccagttaggc tgattctaat ccccagtggc tcctgaggca |
| 29221 | tccagtgcag gtgtgcctct gaacaggctg gtggtcatca caacttgact ctctagtgac |
| 29281 | agagccttca ggggagggga ggccactgac attttcttta ggatgacagg ggcaacagac |
| 29341 | ttgaactcca ccagccacct ctccttttct gccacccagt gaatgaggaa aggcaagaca |
| 29401 | ctccagtgta ggagctagag gccctggaca cagaggatcg atgtccccgc gctctcagct |
| 29461 | cctgatcttt gggagggtgg ttgggctgag taccttacct gggcagtgag tttctgcctc |
| 29521 | tcttcctgga accgctgcct ctcctccagg accttgacct tggcaccctc atacttggca |
| 29581 | gtcatcacct ccagctcccg ggcagtgctc tgtgcttctc gctgcacctc agccagacgg |
| 29641 | gtctcagcat cagcacgtac ggctgccaac tcttggacat acttYtcccg ggcctggtcc |
| 29701 | aactccactt ccagaaactg ccggccaagg ttggcccgct cacccagccc ccggttctcc |
| 29761 | tctgccagca ggccatgcgc cttcttcagc atgctcagct gctctgcata gctggccttc |
| 29821 | tctgcccgca gctgctgagc tgttcgctcc tgctctgcca ccttctgccg cagaggaatc |
| 29881 | agctccccaa gctcccgctg ggcccgcagc agctctgccc gcagtccccc agcggcctgc |
| 29941 | ttgctctgct ccagctcctc acggtggcgt ttctcggcag cggcctgctc ggcctgcagc |
| 30001 | tgctggcaga ggtgcttagc tggcagcagc tcactcacca gggcctgtgt gctggtgtgc |
| 30061 | tcgagctgca gggtggagag ggcctgctct ttctggaaga acttctcctg ccacgccttc |
| 30121 | aattcttggc ccagctcctc cgcacgctca gcctgtgagg tcagctcctg ccgcaggttc |
| 30181 | tctgaagcca cccgctgttt ctcagcctcc tcccggaggc tctgcacctc ctcccgcaga |
| 30241 | gcagagctgc gttctgcagc tctggcactg ttgctggctg tctctgcctg cagcaggcgc |
| 30301 | agcctctcct ccagcttctg gctcttctct gactcggcca tcaccagccg cttcaactcc |
| 30361 | ttgctctccc cctccttctc caggacctgg cgattcagga tggacacctc ctcctccaag |
| 30421 | ctgctgatga ggctattttt cctctcagcc tcttgccggc cccgggccac ctgggccttc |
| 30481 | cactcatctt cagccttgct gtggtcttgt accttggtgc ggaaggcagc caactcccgt |
| 30541 | tgggccgagg ctaaggcact ctgactgtgc cctagctcct gggccttctc ctctaactgg |
| 30601 | ccctgcagag tctccagagc actgtcccgc tcagcccggg aggcccgctc agcctcgagg |
| 30661 | ctgcgttcca ggctgtcagc ctgctcctgc tgcttctggc attgctgttc cagcttgctc |
| 30721 | acctctgccc gcagtgcYtc cagcttgggg cctgttggct ctgtcctgcc agcagcctca |
| 30781 | gattgggctc ctgagccaga tgcgtgctcc ttttctttct tagccagctg ttccttcagt |
| 30841 | tgcttcacgg tttgccgaag ttcctccagc tcttttatct gggctgcctc cagaccacga |
| 30901 | agcttggcca actcctggtc cttgccttcc ttttccgtca gggcatgagc cagtgcctct |
| 30961 | tgcagggtag cgaactccac acgctgctcg ttgagggcgt tctgcagccg catctcaagc |
| 31021 | tctgctctgg ccgccttctc cagggcaagg tcagcctggg cacggccccg ctcctgggtc |
| 31081 | agccgcgcca cctccctttc ctgctgccca cgctcctcct gctgctgccc ctggctctcc |
| 31141 | atcagcgcgg cccgcagccg ttccagctca ttgcYcatct gctctgcctc ccgctccata |
| 31201 | gcctgcagcg ctgcctgtgt gctgcagaac tggcgtccct gttgctcttc cagccactcg |
| 31261 | ggctgtctgt ctcctgccct cgcaggctcc ttgactaact cccgggaggc tgtttcctgc |
| 31321 | tgctcacctg ccttgcgcac caaggtctcc aagcgggcca cctctttgct ggtggcagcc |
| 31381 | atcttttcct gcagagtgga gaggtcatct gcaagcttct gggccctgac ttccttctct |
| 31441 | tggacctgct ggagtgctct ggccaggttg gcatggagct cagctagttY gttctgctgc |
| 31501 | cggcttatct ggagctcgct gtgggattct atgcctgcca ccttctcctt tgcctcctgc |
| 31561 | agctcctggc gggccttctc acattcctcc ttcaaagtca tcagctgttc ctggaacatg |
| 31621 | gcgccatact gtgcctcctc ttgctggcta tcctcatacc gctcacgcca ggcagctact |
| 31681 | tctttgacga gctgctcaca ctcactctca gctgtgtgct gggcaSccat ggcctctgcc |
| 31741 | agctcccgcc gcaggacttc agtctcagcY tgatgggcct ccccaagctg cwgtaatcga |
| 31801 | gcctccagcc ccttgcgccc agccctctct tcttccagct cctttcgttc ccgcttatgc |
| 31861 | tgctccacca ggcttcgggt ctctgccttc agctcagaga tacaacgctg ctgctcttcc |
| 31921 | agggcatctg cagccctgcg cttctcctct tcaaggctgc ccttggtgac cttcaaggac |
| 31981 | tctttgaggg cctggagctg ctcctggagc tggtccttct cctgggccac cctttctttc |
| 32041 | tcagttgctt tttgctgctc agaccgcagc tgcaactcta gctctgcaac ctgggcctgg |
| 32101 | gcctcatgct gttcctggcg ggctgtctca acacaggcct ggagttcctc caccttccgg |
| 32161 | ctcagctctg ccttctcccg ctgggcctgt gtcactgagg tctgggcact gtcccgggct |
| 32221 | tcattagcca cctgaagttg ctgctgcaga atctccagct tggcagcctt ctccttctcc |
| 32281 | agtgcctcca gctgcttgag agccgcatcc cgctccctta aggaggcctc tcgctcctct |
| 32341 | gcagcagtgg ccagttgctg ggcatggtcc tgcctagttg cctcctgctt ctccgctacc |
| 32401 | tccttcaact gctgctcctt ctgcttcagg ctactgctta gctgctccac ctggtggcgg |
| 32461 | aggccctggg aggcctgttc ttgctgttgg agggtctgtg ctagctgggc ctgcttctct |
| 32521 | ttggcctgct gcttcaggcc agccagttct tgatcctgtt gctggatggt ggcattgagt |
| 32581 | gtggtgagct cagaggtcag agaggccacc tgggcagtca accgggcccc atgagcctgg |
| 32641 | gaggcctgct ccagctcttc cttggcctgg ctgaggttgg agatggagct ctgcaggtca |
| 32701 | gtgatcaggc tagacagctg ctgcttttct tcttcgaagt ggccccgctc agcaagcagc |
| 32761 | ttggcttcct gctggcctcg ctcagtctcc agcatctcta ccctggcttg gagctgtgtg |
| 32821 | ttgtttgcag caagagtggc tgcctcttgc ttcaaggttt ccagctggtg gtataaagag |
| 32881 | acaaactggg atcagcatga ctcctcagtc atcaagactc ctctggaaat ggaggaacac |
| 32941 | caggaaccca aatgtactga atgtgagtcc acaccaaccM gcagcacaca ggtctttctg |
| 33001 | tgcattccta cctgcaagac atcacccagc acctcgccct tctcctgggg tgggttatcc |
| 33061 | tgcagctggg acaagtgttc ttccagctgt gaaagttttc cctgaaggat ttcgttcttc |
| 33121 | tcttcaaggc atttctggga gtaaatgaga cccatgtgaa cagagagggg gaacaggcac |
| 33181 | cagattacca cacaagccct cataaacatc agcaaacatg gggcccatca tcttgttacc |
| 33241 | aagatctcct tttgccctgg cctagaaaaa agcagaaacc atggaactgg gagcatgagg |
| 33301 | gacctgattc tctagaccta gttctaaggc acaataaagt cataatgtaa cctgcttact |
| 33361 | acttccccaa ggctgctgag tttacttcaa atttcctYac cgtataccct tcctcagata |
| 33421 | agtcttgagt cctagattat ccaaatcacc accagctgcc caccatcctc tgggcagcaa |
| 33481 | taccactggc agcagctgca gcaaccccca gtcccaaacc cacttcttca ccactgttca |
| 33541 | ctaagcacct acaataggct atgctgggtt catccctaaw tttttttttt tttttttttt |
| 33601 | ttttgagaca gagtctcgct ctatcgccta ggctggagtg cagtggcgag atcttggctt |
| 33661 | actgcaacct ccacctccca gattcgagag cttcttctgt ctcagcctcc cgagtagctg |
| 33721 | ggactacagg catgcatcac cacacccagc ttatgtggga ggcagctact accattcctc |
| 33781 | ttacagagca agaacctgaa gcagatgcta agtgccttga tcaagggcta catggttatt |
| 33841 | agtaagtggc caaattgggt tcttaccaat agtaacaatc acaactggtt cactgagagc |
| 33901 | ttactttaca ctacacgatg tgtgaagtgc tttacatacg ttttctcata ataggtgatg |
| 33961 | aggcagctac tcgttactac tcccatttta gggaaaggat gcttagaaaa acagactgat |
| 34021 | taacttgtcc aagatccaca tcttgtgaac agcagagata ggattgaaag gtgtactata |
| 34081 | aagtgagggc attgactggg gaaaaaaaaa ataagttaaa aaaaaaaggt gtactagact |
| 34141 | gaagcccaca atctttcaat tacagcacac tattgaacct tgaagagaaa gcaacttaat |
| 34201 | acccaaaggc ccagaagagc acagtggtaa actggactca gctttgcaca tgtggtcaag |
| 34261 | actgtggctg tgaccccagc agagggttac cttgtcctgc agggctgcgc tgagctcctt |
| 34321 | ctccagctgg gcctgcttct ctagccactc ctgagtggcc ttgctgtgct cctccgtcag |
| 34381 | ctcattgagg gcatcctgta gctgctgcag atgactggca aactcccgca gctgagacgg |
| 34441 | gcaagtgaga atccccatca cccagaNNNN NNgctgaccc caccactgct gtctgctccc |
| 34501 | agaaccccag gggctggatg cctccaatta tcctgccaac cctctacaaa ccaatcacag |
| 34561 | ccatcacacc tctaattcag aactacaaat caacatttag ggtcacagcc cagtacccgg |
| 34621 | gaagccgcca attatgggat aaggaaaggg gcaaaatatg caggtctctg gagagactga |
| 34681 | agcataagac aggtgctggg acattcttct caaccctctc ttggggagcc ttccagacca |
| 34741 | cctcagccct gaggggcctc catgcacaca ccaccttaac acccacctta aaggaaaggt |
| 34801 | ctccattctc ctccgaaagc tggttgattt tgcgatccat ctggctcttc tctgtcttca |
| 34861 | ggtcctggca ctgcttcagg gtttcatgca gccgcatggt aaggctgcga gggagggaag |
| 34921 | cagtgagaag agagtatggg Ytcctggctg ggtgagggga ggggctgggg cctgggaagg |
| 34981 | aatgcaggga gagctgtacc tctcattctt gtcacgcagc tcctcaagct ccttgggctc |
| 35041 | cagtgggctg gccgcctgct tctcattcag cagggctagg cggtcaatgc gctgctgcat |
| 35101 | catggctatc tgtgcatctg cccagagtgg agggaggagg agaggagaat cagaactatg |
| 35161 | tgcatgagcg gaactatgtg cacagaacta tgtgcacaag gggaagggag gagRcggcat |
| 35221 | agggaagagg aggaatatgg tatccaatct aagggaaaga gaagatgggg acaaagacag |
| 35281 | aacctagctt ccaggaggtc agcctgacca caggcccagg gctgagctct cagctccagg |
| 35341 | ggccagcaga ggggacaaca tgggaggcca ggggacatcc ttccggcaag tctattcaca |
| 35401 | catctgccag tgtgccagcc acctaccctt ctcggtgagg agcttgcggt tctcagctag |
| 35461 | ctccagctcc agctcatccc tattacttct ctcatcagca agctgcttct tcagccgtct |
| 35521 | catctggaac tgtggggtct gcaggatatc acccatggga gaagctggag aacctgagag |
| 35581 | aaagctgagg agggagaagg cccatatgca ttggtaagcg cctaagatSt gaagcaatca |
| 35641 | gcaacaggca gcagtaaggg agcggggtct atggaagtgc gcactagcag ctgggaggcc |
| 35701 | tgtgcacctg ggttgtttgc agtgctacct caagcctcat gacatgatct tgggaaaact |
| 35761 | tttcttctca ctgccttaat ttctttgtca gaaaaaaaaa aagtgatata ttctccttgg |
| 35821 | ctctgcagag atgtgctaag agaagagact tcattgtagt ctcYgaggaa gaagggtatt |
| 35881 | ccataaacag ggaacagctt ttgagggagg ccttctgcac atgacaaatc ccaaccactt |
| 35941 | cccccagtta atactgggtg aggtgaggag atcccagatc taccggatac tctaagcact |
| 36001 | cccagaggct ggttaacatc ttaactctag aagttaggaa tgtgaggaat ggatggatgc |
| 36061 | tgaggcccta tcccaggagg agaggcccag aacatacttg ttcccactgg aagaggaggc |
| 36121 | aaccttctgt agctctagga agcgaatctc cctcttggcc tggtggctag gtggggagag |
| 36181 | ctcttcaggg aatgtgctag aacaggtaga aggcacagga gctgggggta agaaatagac |
| 36241 | aaaataaatc aacaaaggtt agtaagaagg tgwaaagatt tgcaccttta tgcaatttga |
| 36301 | gaactcgcct tagtgggagt atatccatgg atacttatat gtaaactacc aaggaaggca |
| 36361 | ctgggcagaa cctgaaacct ggtgcctggg ttatccagag ttccgtgtct ctaaccaaaa |
| 36421 | tgaagtgcag ggcttccatt tctacagagc atcattactc agagcacagt ggagtgacgg |
| 36481 | tgtcctctgg ctcagaaact gcaccaagtg ctcaagcttt ttcctcgagt ccacactgca |
| 36541 | gagacggctt ggggagccct gagcaggatc tccttgcagc ccccaacaac ctttcctatc |
| 36601 | agagccattg gccaccattt ggaattcatc ccccctgaag ccttgcacct ccaaaatgcc |
| 36661 | ctcctcctca aaacccttct ctttccgtgg tttccgtgac cccttttgta ggactctcct |
| 36721 | acctctaatc acccattctt aagtctcctc atctgctcct ccctcctcta cccaccctct |
| 36781 | aagctcccag agatctgatg gggcctcttg gctctgccca tagtgcccac ggcttcagtg |
| 36841 | gccatccaat gctgacttgc aaacctatct tctggtccag agcctgctcc tgaattctgg |
| 36901 | actcacattt ctaacatctc cacctgaagg tctcatgagg aacctgaata caaggaacat |
| 36961 | caaactcaac ggctagaact cgcctagacc tccttcttac attcctatca tccaccagct |
| 37021 | gacctcttca tcagaagacc agcagaacga tcactctttc tactgtagta ccagccgagg |
| 37081 | ccattgatat attccgtact tagattcctt atcattattt cctggtttat gtatctgcct |
| 37141 | ttccacctag aacatgcact acatgacggg aaaaacattt tttaaaagac tgtgggccgg |
| 37201 | gcgcagtggc tcatgcctgt attcctaaca ctttgggagg ccgaggcagg tggatcacct |
| 37261 | gatgtcagga gttcgagacc Rgcctaatca acatggtgaa accccgtctc tacaaaaaat |
| 37321 | acaaaaatta gccaggtgtg gtggcatgcg cctgtaatcc cagctactcg ggaggctRag |
| 37381 | caggagaatt gcttgaacac gggaagtgga gattgcagtg agctgagatc gcaccactac |
| 37441 | actccagcct gggcgacaga gagagactct gtctcaaaaa aaaataaaaa taaaaataaa |
| 37501 | aagactgtat tctcagcttc taagcctaga gtaatgattg tttaaaaaat aaaaaacaaa |
| 37561 | agtaggtatg atcacttgta aatatcctac ataactgctg ccaggcagca aggaggagct |
| 37621 | ccataggaac tgagatgata caacatggcc cagaaatcga cctactccat tacacacact |
| 37681 | tttcctgagc atcaactctg ccttgtgttg gggacagaaa caaaccccca ccatacttga |
| 37741 | caactcagga tcctagtatt ccccatttca gagatgaggg gattctcagg agtgtgtacc |
| 37801 | tacttacctt tctgtaggaa gttctctagg tcctcattaa ggtttagccc gtcctcatgg |
| 37861 | tccagcacaa atttaagaat gacagccaac tcagcctggg ccacagggag aaaaagagca |
| 37921 | tcacttaggt gttaggcctc tgaagggatg gctgccagga gctccaccct ggcagtgcca |
| 37981 | gtccccggct ccctcatgcc ctaggagcct gctggatccc attctccctc tcagagctga |
| 38041 | ggtcctatga cccagccaac aacattcaac tagagatatg ttcaccccac ttgtcccctt |
| 38101 | ccctcccacc tcccctcaag atgtaccgaa gtcagagttc ctgactgact taactcctga |
| 38161 | ccacaagtga agtgactgtt aaggaaagta acacaatcag aaagttgctt ttattgttat |
| 38221 | tgttgttaag aaacaagatc ttgctctgtc acccaggctg gagtacagtg gcacaatcat |
| 38281 | agctcactat aacctccaac tcctggagtc aactcctcct cctcagctcc ccaagtagct |
| 38341 | agaactaSag acacacactg ccacacccag ctcatttttt ttaaaaaaaa tattttttgt |
| 38401 | tgagacaggg tctcgctatg ttacccaggc tgatcttgaa ctcctggcct caagcaatcc |
| 38461 | tcctgtctcg gcctcccaaa gtgctgggac tataggcgtg agccaccacg cccggccagg |
| 38521 | aagcttttat atataactat gttggcctct tccagatgac cctaccagag gaaaggatga |
| 38581 | gataaatgaa gtgaaagttc tgtggggRaa aaaaaacaca cacacaNNga aacacatata |
| 38641 | ggaacagaga ataatttcta cgtattatcc taaaacacat tacagttaaa catatgcaca |
| 38701 | taaaaaagga cttgaaagta catcctttaa aaagttagca aactctggga atgagtttat |
| 38761 | cggcaactgt tatttcctcc tgcatgcttt tcaacgtttc ccagactttc tatgatgaat |
| 38821 | ggcaataatt gctataactg tatttttaaa aagtccagta tctgctgttc aaagaaaaac |
| 38881 | aagtcaggtg aggtgaagca tgggctaggc ctgctaggtg gcatggaggc acaagggctt |
| 38941 | acctgaattt tatattcaaa ctgttcccag tccctgggac ttttggagct catggtagag |
| 39001 | tggtataaga gcagcatggt catctagaag ccaaacagga ggcagtcaMt gagtggggct |
| 39061 | gtctctcttc tcagtgYccc cattctgggc tgttctgggg actctgagct attccggtga |
| 39121 | ctcttctaaa agtcctggaa ttcagcaatc ccaggccacc atccttaaca gctccaggaa |
| 39181 | acactttttt tttttttttt taattaagga tagtgtacac tgtacaaaaa gctgagcgcc |
| 39241 | aagattcctg catggctcaa ttcctggact catggaaccc tgggtgtgaa gctttaggta |
| 39301 | ccctctatag gccagcaccc agaaagagca gagggaatga cttaagactg agctataata |
| 39361 | aaaatatggt gatagccgaa tgagacctga tttctgccag gtctccaccg aggcccacag |
| 39421 | taatggtcta gtaaaccatt gtttgtttca taatctctgt cataagatca tgggctccta |
| 39481 | gaatccttgg gctaaattag cctctgtctc aagacaggtg gctgcctggt tctacctcca |
| 39541 | acgtactaga gccccagaaa taaagcaccc atcatctccc cttgggttaa gagaaggccc |
| 39601 | tgcagggtcc ctcaggtacc atcaccccaa ccctgccctg cctccccagt ctggtattcc |
| 39661 | ataggtacct tcgccagttc cagctctgat ccctctagca ccttctgtgc agataccagg |
| 39721 | cattctgggg aagagggatg ttttcgattt tctgcaatga caacaacgYa gaaaacaggg |
| 39781 | tgaacttcct ggaaaccata cctgagatcc cagaacactg aaatctgggg agcagaatgg |
| 39841 | ctatctctgg tggggctatg agatgtaggc ctccttactc ctatggcctc tttcccctgc |
| 39901 | cttacaattt tccatctgaa gtcagttcta tcctgggtca atccaagggc tacagggata |
| 39961 | atggaagaag aaagcagcct cggaattcac agcaactctg ggtcatgatg ttgcactctc |
| 40021 | ccacccactc accaactctc agcaccacag aggaagagcc cagagatgcc aggtcacttt |
| 40081 | agaatcctgt tccaactgag gtcacagagt aaacctatag ctgagtcaga aaaggacaac |
| 40141 | tagggatacg tgactcttat actgcccgag ggtctgggct caaattacta acaccttact |
| 40201 | ttatctaaat aaagcctcac ctttcctata attctctagg ggtggagatg gcagctagct |
| 40261 | ccccatgtca aagacgagga ctctgtggtc taaggctgga caaggggagg ggaactgaaa |
| 40321 | agcactgtac tagaagcttt gcatgtgttc ttatttaatc cacccatcca cttgaagagc |
| 40381 | agttatctcc atattataga tagggaacct atgtccagaa aggtcacaca agcagtgagg |
| 40441 | ggggtcaaga tcaaccttaa tgtgcctctg cccctacacc tatcgcctaa gcaattaatt |
| 40501 | caaagttatc tgggaaaaac agtttttctt gcaagggaaa gagactcatt ctctNttctg |
| 40561 | tctccttgaa gttcggggaa acagaaactt aaggtgctca aagagttaaa agtgacaggt |
| 40621 | cccaaaaagt aaccttctgg ctccacctag atctccgaga agggctctgg gactgaaggg |
| 40681 | acactcttct tacaccaact gatgaaggca tcacaagacc caggggatct gaaccagctg |
| 40741 | ttttcatctt tgtgttaaag agtggggagc cttgaaattt ttctcctcca gacacccatg |
| 40801 | aactagttcc tctggactct ccaatcccac gagctgagga agcagcttct tcatagctgg |
| 40861 | ataagaaagg tgcttactct gcagaaaact gcacacaaag tccagtctct ctgacaccgg |
| 40921 | ctgcttcaag atttgctgtc cctcttcagt gccatggcta gaaaaagagc aagtattagg |
| 40981 | accagagtat tcagcaatct ttgcctcctt gctgctgcat ttcatagtac ctccccattc |
| 41041 | cttccagtct tgatctctgc tggatctgga agagatccag atcagatcct ggaggcaggt |
| 41101 | gacagatacc aggacaccat ttatccagat ggaactgtgt ggtgggtggg aagaggggac |
| 41161 | ttgaggcatg gggttcttct ggaaaagatg gcactctgcc accctatttg caaacataaa |
| 41221 | aaagctctgt ggaaaggggc cgagaggaga Yggaggcagc acagatgaaY gggaagctat |
| 41281 | attacaccat ctggaatggc tcaactctaa actgtcagga agcctggaga acacacctag |
| 41341 | gttaagagtg agagaagacc atggggaggg aaaaacatcc aagaactgaa agaacaggag |
| 41401 | gaaacaaggc tctgaagtga cagggtgaag ttcccccact aggctgaagg ctgagatggc |
| 41461 | aaaagcctcc cagaggagga tcccagggac tagagtgttg tttgtttgtt tgtttgtttg |
| 41521 | agacagaatc tcgctctgtt gcccaggctg gagtgcagtg gcacgatctc ggctcactat |
| 41581 | aacctctgac tcctgggttc aagcaatcct cctgcctcag cctcccaagt agctgggatt |
| 41641 | acaggcatgg accaccacgc ccggctaatt tttgtatttt tagtagagat ggggtttcgc |
| 41701 | catgttggcc aggctggtct caaactcctg acctccaccc acctcagcct ctcaaagtgc |
| 41761 | tgggattaca ggctggagcc actgcgccca gctaggacta gtattcttga ttagtggttc |
| 41821 | ttgtctgtac ttgcttaact ggaagagcgg gcagtgtgaa gagagccatg aggagaggga |
| 41881 | gacagcaggg ggagctctca cacagcccag gaggctgagg caggagaact gcttcaaccc |
| 41941 | aagcgacaga gattacagtg agctgagatc atgccactgc actccagcct gggcaacaga |
| 42001 | gcgagattct gtctccaaaa aaaaaaaaga atactagtcc ctgggatcct gctctgggag |
| 42061 | gcYtttgcca tctcagcctt caacctagtg ggggaacttc gctggatccc ctagctaact |
| 42121 | gcagttccat tttcagaagc agcaggtgaa ataccacttc aagttagaaa gctgaattct |
| 42181 | ctaggctaca gtgggagatc ctgattagtg ggccccttgc ttctcagaat ctacactcct |
| 42241 | agttcttagc caactttctg gagggcaggg caggcccaga accttccaat gccagagaca |
| 42301 | tcctgcaagc ataagcatca tccaagccaa gactatgagt gtacctcccc ccatacccca |
| 42361 | tgtgattaca caggcttctt ccaagtgaat ccctcccaga agcactctgt ctggatagtt |
| 42421 | ggtctgagat cagagactcc tacaaagtct tgaggagatg ctaatctctt acagactccc |
| 42481 | aaatgagcca ttttccctac ccacccacaa tctggaatgt tgtgactcct tggctcagtg |
| 42541 | tagagcaata tttacctcag agtattccat taaYtaaaat aaaaagaggt tctcaggtga |
| 42601 | aatgtttaaa aaatgctggg ttaaactgaa cattttttat agaagaagat acaacatgca |
| 42661 | ttatttccta aacctatttg accacaaaac ccttttcccc caccccctac aaagcctggt |
| 42721 | gtaagtaaag ctgcatggag catactttgg acaacagtag tcctagagaa atccagccca |
| 42781 | tcctttaaga ctgtgttgaa gtctcatctc tgtcaggaag ccatccacaa ctcctgccag |
| 42841 | tcatgcctcg gctgtcctcc aggtcttcct gcagcactca tgaatgcaac tgcaccattt |
| 42901 | acaactgccc aYtccaactg cactagctga aagccaggaa taaagtaggt gctcaataca |
| 42961 | tatgggctga ctgacaactg agggatgctt tttactgaga agagcagaaa taaaagtcag |
| 43021 | caagacaaaa atgtcagaMc tStaagtttt ctggcaaaat gcctccacta tactctgtta |
| 43081 | ttcaagcttc acagctatca aacagacggg acagcctttt ttgctttaga ctattcacgc |
| 43141 | acagaagaac caaaagggaa acttgtcaga ggggttgatc ccagttaatt ttcccagaga |
| 43201 | gaaggccaag tggccaagca Rttaaaccct caactcccac agcttatttt ttttttaatt |
| 43261 | ttttaaagaa ttatttttct cagggtgccc cttgtccagt gtgcagacag gagccaccca |
| 43321 | actcccacag cttctatcta ccctccaacc ttgccatcct ctgctgcctt ctgctgagaa |
| 43381 | ctaggctgct gatttaggtt ggcaccagta gcggtatcag aaatgtaaga gccagaacat |
| 43441 | attaactaag aacctccatt tcccaaaagg aagctggtct gcattgtcct ggatctgaga |
| 43501 | tggagattag cattaacYtg ggatttcaat atcgcctacc tccaagcccc acttctactg |
| 43561 | cagcctaggt tcaggcaaca ccctagaaga caacttgcaa atgagcatct tgaatttttg |
| 43621 | gcccttccta tgaaaatttc ctaScaggtt ataaacctac agtggctaac ccaaggacaa |
| 43681 | cacacatcct tactcttggg cacatctact gctcttgtca ccaggccaag gaaatttctc |
| 43741 | tcttcaaaag caaaagttcc ttgatctatt ttgaagtcaa aaaagcatgc tgccccatat |
| 43801 | tgtgtgttac aatacaaggc atattatacc tggctgcttc acttttagtg aggttaaaat |
| 43861 | taaacagaga gttaggtatt gccagactaa tggttttacc acccattaat gtcaggtgcc |
| 43921 | tggatctatc acttccttag ggtttgcaaa ataatgattt tcaacagaag ttgcaaaatg |
| 43981 | ctgacttaca ggtggtacac aaccccaact atgaagtatc cttgacaaaa aaaaaaagtg |
| 44041 | aatcaagctt agatctaatt atcagtatat aggcaacaag gggaatagtg gaacaagtta |
| 44101 | agccgtacca taataatgca attaacaaaa tccaggattt aaggatttct ataagattaa |
| 44161 | aaaaaaatga ggtggtgtcg agtggggaga gaaaaaagca ggaaacaaaa ctggtgagag |
| 44221 | gaaatgaccc cctgatgaaa gatcttaaac accaggctga agattttaga tttctaccta |
| 44281 | ttagaaatga atattcactg aggtttgatg aagagtcact gaagtgtcac aaagaaaaca |
| 44341 | agatttgaga aagattcttg agaactcgtg cataggaatg aactgcaata agggcagatt |
| 44401 | agagaagaac taggccatga gggcctagta tccagaatga ggcagaggga gggacgctgg |
| 44461 | atgtgagcag agaggatgat gaccaattag agatcagcgc caaggagaca gagaagtcaa |
| 44521 | gatgatgttg aaatgtcctg cctgggtaac tgggagagaa aatggtactg tctggaccaa |
| 44581 | aataagaata gtgacttctt tcagaggtac taaggcaggt cctggttaga gccatatgga |
| 44641 | atattaaaaa atcctgtaag ataacccacg atcaacacac acttcaaata aaaaccactt |
| 44701 | tagcagcaga cccatctgtc ctgggccact tggccccgct agaccttgct gatagagaac |
| 44761 | accaactcat ctcttcactc aacaaacatt cactaaaacc taaacaatgt agcatcctaa |
| 44821 | gctaggtagg gaggacccca agatgaatca gacacagttc tagcctttga taagcttaat |
| 44881 | tctaccttct ttccctatca atgagctgtt acctatgaac aggtagctaa agtgcttgct |
| 44941 | tcagagatat gagtccctga taccagttcc taactgccac Rtatggcaca taaacacctt |
| 45001 | caacacttca gtYtatcctt ggcctggggc aaacaatYta atgtgctttt tcttaaaaaa |
| 45061 | aaaaaaaaaa aaaaaaaaaa aagatgacaa ctgacctatg cttggtccca gaaaggtagg |
| 45121 | atactgggag gaagccccac tttattcatt tccgagataa ctgtgacgtt caaaggaaat |
| 45181 | gtgctaaaga cacagaccaa aaggccaaac tacaatcctt gctgcatggt aacaacaagc |
| 45241 | ttattaaatg ctcatggacc tcagaaatca agttaattga agcagtaaaa accccagaag |
| 45301 | tgtacttatt ttataatggc ttagaaagag gcatatctgt actgtgctaa caagttagag |
| 45361 | atgagatttc taggtgtttc ttctttcctt cagatgtgag agcttcccct ttcttccctc |
| 45421 | agaaggctgc aaagagggct tgtggcatag ggtagggatg agtcaagaaa ggagggttgt |
| 45481 | agaagccttc tggtgagaca agagatgggg aacccaagag tgagcacttg gaagaatgtg |
| 45541 | agggcgtgga gccttggagg ttctcctgtg caagaatgca gaagcctcag gatgggacaa |
| 45601 | gcgtaaaacc tctcccatgg agtggtacag caactggaag atgaggtcaa tgtattcttg |
| 45661 | cctttaggcc ccaccctggc ttatgatgtc ccaatccttc ccctgatttc tggtcatagc |
| 45721 | catcatcaag taaagagaac aagtacagcc cccaccccag caatcagctt tgccttagag |
| 45781 | gctagaaagg ggtatggtgc gtactcacat tctgtcaatg atcttgatga agatgctgca |
| 45841 | gtcctggagc tgcagcacag cctccacagg gtcagccacg tgtagactgt tcacctgtaa |
| 45901 | atcaaaggga acagcctagt gagaccgtta gaagcaacat ggtttgctcc ttttcccaga |
| 45961 | ggcttaatgt gagtgcttac agttgtagac tataggaata caagaactga tgattctcag |
| 46021 | gtttttaatt tcaggaagga gtgccgttgc actcactcat ccagttacag ccacctgaWa |
| 46081 | tgcaaccatc agagagccct tcctttggct tcaactaaat aaaagcttaa actttcctaa |
| 46141 | gatgttccag cactgggagt attttggaag gatacaccaa taaattggta ctaccactgc |
| 46201 | ttctaaagag aggaacttga cagcagggag tctgaggtgg gaaaagaaag gctcttacat |
| 46261 | tataactctc tgtagctctt gaatgtatta tagctctttg tatttcttga acgtttacat |
| 46321 | tataagtttc tgtatccctt ggatttttat attatagtcc tttgtggctc ttgagttttg |
| 46381 | aaatatctat tcaaaactta aatattacat atatgctata cactcccaca aacatataaa |
| 46441 | atcttattaa tacattttat attatagccc tttgtggctc ttgagttttg aaatatctat |
| 46501 | tcaaaactta aatattacat atatgctata cactcccaca aagatataaa atcttattaa |
| 46561 | tacattttta ttctttccac acataaagct ttggctgagg ggagaaggga aaaacaatag |
| 46621 | gggaacaaga tgatttttta aaaagggggt ggagggagcc aagcgcggtg gctcacacct |
| 46681 | gtaatcctag cactttggga ggctgaggcg ggaagatcac ttgagctcag gagttcaaga |
| 46741 | ccaacctggg caacatggtg aaaccctgtc ctacaaaaaa tacaaaaaat tagccaggag |
| 46801 | tggtggcaca tacctgtagt cccagctact tagaaggctg aggtgggagg atcaccttag |
| 46861 | cccagggagg tcgaggctgc agtgagctgg gattgcatca ctgtactcta gcctgagtga |
| 46921 | cagagcaaga ccctgtctcc aaaggaaaaa aaaaaagaaa agaaaagaaa aaaaaagggg |
| 46981 | gcggtggggg atgaaagaat aggagctggg aatttcaaag aaaaacttct taattctaat |
| 47041 | ttagttgcct gttcccattc tttggcttca gataggcata catttaaatc agtttttacc |
| 47101 | actagaatat tcaccagggg tcacacacat caggtacaca ggttattcaa taagtataca |
| 47161 | tggtcaacct ctacaggaaa acctcatgaa ttccacaggc actacagttc agaagtagcc |
| 47221 | tgggctgaag tttacgtttt tattactata ggaagaaagg gtttgctaag ttaattattc |
| 47281 | acatattcaa cactaagagg ggagagagac tgagcctgct ttaagaaaca aggtatttta |
| 47341 | aagaacttca gcatagtggt tatgtgcaga caactagatt ctattgctta caaatgtgtt |
| 47401 | ctctatattt gggctgggag ccagtacata cacactttgg gcccagctcc tccacaaact |
| 47461 | tgccagcaac ctttgttaca aactttattt ctctctgcac tagtttcctc ccttgccaaa |
| 47521 | tggatggaat ccctacttta tctacttcaa aggaccagtg tgagctcaaa tgggattatg |
| 47581 | gatgcaagaa cacattaaaa aatataaagt aggccaggtg cagtggctca cacctgtaat |
| 47641 | cccagcactt tgggaggctg aggtaggggg actgcttgag gctgggagtt taggaccacc |
| 47701 | ctgggcaaca taacaatacc ttgtctctac taaataaata aataagcaag caagcaagct |
| 47761 | ggacatggtg acatgtgcct gtaagtccta cctacataag aggctgaggt aggtggatca |
| 47821 | tttgagccca ggagctggag aagttacMat gagctatgat agtgtccctg cactgtggcc |
| 47881 | tgggcaacag agacatctca aaaaaaaaaa gaaaaaagaa aaactaataa acataaagca |
| 47941 | ctattatggt taataaaatt aaaataatga agtagacata acatttgtat ttcttaatag |
| 48001 | ttaagttagc tgcatgcact atagaattaa gaggattttt aaaaatcaat gatttctcta |
| 48061 | cagacatgat agtatttatt tgagttaaaY actttcacat ttaaaaacaa cttttaggcc |
| 48121 | aggcgcagtg gcccatgcct gtaatcMcag cacttgggga ggctgaggtg gaaggactgc |
| 48181 | ttgaggccag gagttcaaga ccagcctggg caacataagg agaccctggc tctacaaaat |
| 48241 | aataatttaa aaaaattttt agctgggtga ggtggcttgc gcctgtatgc tcRcgtagtc |
| 48301 | cctgctactc gggaggctga cgtgggaaga tcgcttgagc ctgggaggtc aaggctacag |
| 48361 | cgagctatga tcacaccact gcactctagc ctggccaacg gattgagacc ctgtctcaaa |
| 48421 | aaaataaata aataaataaa tacaacattt aggctaggtg tggtggctca tgcctgtaat |
| 48481 | cccagtgctt tgggaggacg aggagggaga atcgcttgag gccaggagtt tgagatcagc |
| 48541 | ctaggcaaca tagcaagacc ctttccccaa aaaattaaaa aaaaaaaaaa aaagagagag |
| 48601 | aaaggaaaaa tagcccagca tggtggtgca tgcctgtggt cctagctact caggaggcta |
| 48661 | aggtgggagg cagaggattg cttgagtgca ggagttccag gctgctgtga gctatgaagc |
| 48721 | tgcaactgca ttccagcctg ggtcacagag caagacccta tctcaaaaag Mggaatcttt |
| 48781 | tagatgaatc ccagttaaaa cagattaaaa ttttttttaa cgtgaaataa aatatgactt |
| 48841 | attgacgtct taaagagttt gctaatatat ttataatata tttttactgt cttttgaaaa |
| 48901 | aagctcaatc taattggtca ggacaggtca ttgtaagttt tctaggaaga Wtctgaaaac |
| 48961 | ttcaggccaa gattccattt tacctgcaga aaatgaagag actgccaagg ctaaaccatc |
| 49021 | tcctatcaat acctgcaatc taggtaaaac attcaaggac tatggatgct tgttcactaa |
| 49081 | caagactaaa aaatcatacc aattagctga tgtaaagtct cctaaggaat ctggattcat |
| 49141 | cacatatacc aattaagtaa taatccagcc acagttcctt tcatggatat aaaaatattt |
| 49201 | tttcttcctg tgagttattt ttgctctcaa gtgagaaatc atttaaaagt tgaggaagta |
| 49261 | gagaagttcc ttcatctcta gtcagggcat aaaacatgag agtgaggacc aggctcacca |
| 49321 | cataactcaa gagttgcctt taaggattgc aaggttgctt aaggttgtca aatcttaaaa |
| 49381 | taccctgtaa cagtttattc attattaact gctgtttaca gaatagtttc aagctataat |
| 49441 | ttaagtatta ataaaataaa ccactaattt atttttatgt tataaattac tgaaaattca |
| 49501 | gaagacaaaa aacagagctc ccaataattc tagtatttta atacaggtac ttttagcatt |
| 49561 | ttgatgtatt tctttcgttt tttctaggta atttttttta aactgtagtt ggaattatca |
| 49621 | tgtgtgtgta tgtgtatgtt ctatgccact cttctcacta acataacaga tgcattattt |
| 49681 | catgctacta cagtttcata aaatttgctt tagctccaca acttttgttt aaattgaaaa |
| 49741 | cattctctat actatattta tatatatttt taagagacag gctcttgcta tgttataggt |
| 49801 | tggagtgcag cagctattta cagatgcaat catggctaac tgcaacctca aactcccggg |
| 49861 | ctcaagcaat tctcccatct cagcttccca agtagcaggg actacaggtg tgcactactg |
| 49921 | atcccagcac atataactat actttaacat tttcatgcat gttctttgtt tKtttttttt |
| 49981 | tttttttttt tttgacacag gctttcactc tattgtccag gccggagtgt agtggcacga |
| 50041 | tcacagctca ctgcagcttc aacctccaca gcctcaggtg atcctcccac ctcagcctcc |
| 50101 | taagtagctg gaaccacagg agcacaccac cacacccagc taatttttgt attttttgaa |
| 50161 | RaRacagggt Ytcaccacgt tgcccagggt ggttgcaaac ttctgagctc aagtgatcct |
| 50221 | ccacctcagc ctcccaaagt gctggcatta caggtgtgag ccactgcacc tggcctcatg |
| 50281 | catatttatt gtgacttttc agaaaaggat gtaagtagct gctataaata aaactagtct |
| 50341 | taggccaggc gcagtggctc atgcctacaa tcctaacact ttgggaggtc aaggcgggtg |
| 50401 | gatcactgga gctcaggaat tcaagaccag cctgggaaac atggcaaaac ccagtctcta |
| 50461 | ccaaaaatat aaaacattgt attttttttg aggtcacatt aaaattgagg tcagaagttt |
| 50521 | gagaccagcc tgggcaactt agcaagaccc catctctaca aaaagttttt taaaattagc |
| 50581 | atggcatgta cccctgtggt cctagctact tggggggctg aggtagaagg atggcttgag |
| 50641 | ccccaaagtt tgaggttaca atgaactatg attacatcac tgtactccag tctaggcaac |
| 50701 | agaatgagac tctatgtcta ttttttaaaa aaagaaaaat ggttggctct ccaattggcc |
| 50761 | ctgattaatc agtactcagg gtgaatacta aaatatcctg tgtcaactag catgcccagg |
| 50821 | aagtatttta aatgtcaaga actatgattt attgagataa catgctaaat gactgcatgt |
| 50881 | atgtgggtac ttgtgcacac tatatgtatc tcattatcct tgtaattacc ccatgagata |
| 50941 | ttttatttta ttttttcatg gtgccaagag aaattagaac tttatgaggt attttaattt |
| 51001 | aaaatcatcc ccattaaaaa aaatgatact ggccaggcgt ggtggctcac acctgtaatc |
| 51061 | ccagcacttt gggaggtcaa ggagggcgga ccacgaggtc aggagatcga gaccatcctg |
| 51121 | gccaacatgg tgaaacccgt ctctactaaa aatacaaaaa aattagctgg gcatggtggc |
| 51181 | gtgcacctgt aatcccagct actcgggagg ctgaagcagg agaattgctt gaacccagga |
| 51241 | ggcagaggtt gcagtgagcc gagatcacgc cactgcactc cagcctggYg acagagcaag |
| 51301 | actccatctc aaaaaaaaaa agatactgaa gatactgaga ggccaacttg ccctgggtca |
| 51361 | gagagaccag tgctgtgatt ctatgctggg catgtgactc tgagacccat atttatcacc |
| 51421 | acactccatg gtctcctctg tttctttagc tttatttatt atttttatat atatttttga |
| 51481 | aacatggtct cactctgtcg cccacgctgg aatgcagtgg tgcaatcatg gctcactgca |
| 51541 | gcctcaacct tcagggctca agggatcctc ccaccttagc ctgccaagta ggtgagacta |
| 51601 | caggtgcacg acaccatgcc cagctgattt tttaattttt tgtagagaca ggtttttgcc |
| 51661 | atgttgccca ggctggtctt gaactcttgg gctcaagcRa tcctcctgcc tcagcctccc |
| 51721 | aaagtgctgg gattacaggt gtgagccacc acatccagcc tctctagctt taaatatcca |
| 51781 | atgcactcta ttattttttc atttgccact gtctctggta gaaaatgtat gaatgatcta |
| 51841 | gcaaaagaat tttgatgaag taatcggcaa ggcaacattt gctgatggac aatggcaaca |
| 51901 | gacagaaagg tggtaagaga gtgatgagaa ccttgtccta gattaaaggc ctttgaagac |
| 51961 | caaagtccct gcatatgttg atcattaagg tcagcattta ccacYccccc tttttttttt |
| 52021 | ttaatttttt agaccgagtc ttgctctgtt gcccaggctg gagtgcaatg gtgtgatctc |
| 52081 | ggctcacagc aacctccgcc tcccgggttc aagtgattct ccggcctcag cctcccgagt |
| 52141 | gactgggatt acaggtgccc gccaccacag ccagctaatt tttgtatttt tagtagagac |
| 52201 | gggggggttt caccagcttg gccaggctgg tctcgaactc ctgacctcag gtgatccacc |
| 52261 | tgcctcggcc tcccaaagtg ctgggattac aggcataagc tccgcacccg gcccacattt |
| 52321 | acccttttat tcagaaaaat acacacagag cagatgaaaa tgcatttcca tgctgtctaa |
| 52381 | aggaaaacta tttagtctag aaatgagaag actttacata gccctggctc ctacaaaact |
| 52441 | cctctcctaa ctctctcaag cttcctggag aagtatagcc aacaaaagag aggaagacta |
| 52501 | cttacccaag agaggagtgc agccccccgg gtggcgtgga gtgtcatctt ggtgatgcca |
| 52561 | gacagtcact ccaatgcgcc tggaacccaa gagaggaaga aaagcagtta atcatatcag |
| 52621 | atatccatga tcaagaaata aaggcgaaat ggttcaattt gccaattctc aacacttaaa |
| 52681 | tcactcttca ctgaatccac caagacacca tgggaatttt tagaaagctg agtacacatg |
| 52741 | acaattacta tagagcttac atcagagacg acaggaaaga attgtgcatt taactcaatg |
| 52801 | tctaccccat gtaatttttc agttccacag aaaatacaac atccaaagct ggccttcaac |
| 52861 | cctactcttt gggacatgat gcccttaaat atggaacaaa catggttttc agacagacct |
| 52921 | gggtctggat gctgatttaa tgtgaccagg gtcataggac cttagacaag caacataacc |
| 52981 | tctttYagtc tttttcttta tctgcaaaat gggatccaca tacacctctc tgggttattg |
| 53041 | tgatgcttaa agataaagca aatagtacgg tgcctggcac atggtaggtt gtcaataaat |
| 53101 | aattactgtt cttaagcaga aatgttgatt tctagctctt tctacaggta cccaaattgt |
| 53161 | acctaacaat acctaagtcc ttggggaaac tgttctagtt tctggatttt tatatcctcc |
| 53221 | acagaaatgt ctcacaaaca cagttcaaat gggggattga gaatatccaa acttaggtct |
| 53281 | ctggatgagg tcgagcccaa ttccggagga gatcccatgg gacatttaat gaaaattaSg |
| 53341 | cacctgtcct ggtgttctca tccccaattt cctttcccat tgagattctg ccacctgcct |
| 53401 | gccaacccac ccaagagacc acagaccaga ttaactaaat attcaccaac acctcctagc |
| 53461 | aacccttgca attttcacta atctccaggg gcacaatgag ccctgccccc atctcctgcc |
| 53521 | acacgtgctc agtctgtaat tactctagaa ggaggaacaa gagtggtaag agccacatca |
| 53581 | cttgtaagtc tccccatata ttagcatctc cagagtgtgg gctctgtgga accagtctca |
| 53641 | tgtatgcaag ttggggataa agaaagacac cattcttgcc cagaaggtgc tcactgtcta |
| 53701 | gtaMgggaaa gagatacgaa aatctgaaaa ttcactcccc attatctctt ggtccttcct |
| 53761 | atccaagtat actctattta tcagccaacc gaaaggtatt ttcttagttc ctaaatatga |
| 53821 | tcatcctcgc tgggagcagt ggctcacgcc tgtaatccca gcactttgag agaccaaggc |
| 53881 | gggtggatcg caaggtcagg agatcgagac catcctggct aacatggtga aaccccgtct |
| 53941 | ctactaaaaa tacaaaaaaa ttagctgggc atggtggcgg gcgcctgtag tcccagctac |
| 54001 | tcgggaggct gaggcaggag aatggcgtga acccgggagg cggagcttgc agtgagccga |
| 54061 | gatcgcacca ctgcactcca gtttgggtga cagagcgaga ctccgtctca aaaaaaaata |
| 54121 | ataaaaaaaa agatcatcct gtgctagaag ccacatggaa aatgggagac aatgacatgg |
| 54181 | tccttgcctt tgaggggctt atagcagggt agggagtaaa attaaacata tgtgaagcac |
| 54241 | taacagaaga aaataattaa atgctaaata taactaaatg ctaattacaa ttaagtgctt |
| 54301 | cctgaccctg caacatccaa catgattcca catgtcatac cagcatcagt cttggaattg |
| 54361 | agccaattca cctgggcttt ggcaagggca aatgaaatgg ttaagagagc tctcaggagg |
| 54421 | gcagaaaagc taaggggctt atgttcctca gtcttggata attatcatca agtataccct |
| 54481 | atccatgggc aagattatgt agcccttgag gccaggcatg gatggatgca gatagcccca |
| 54541 | actactgcac tgcaataagg cttttctgtg gctgacaaga ctaaagctcg agggaccaca |
| 54601 | cttggctctg tggtSgggcg gcaacagcca gacactcaac tggggtttct ttaaaaaaaa |
| 54661 | ggcctctgag ctattaaatc acaaggaatg aggctgagat caccgctccc ccatcctcac |
| 54721 | tgctgcctat agacttgcag tccttttgat gaggcccaac acttcgaaat tttatctgca |
| 54781 | actgtaggca gatcttgccc cctttgaaag actcacagct gtgctactaa aaatgtcctg |
| 54841 | aggtttaacc aacctcttca tctcagaggc aaaacataca accctgaggt cctcccagga |
| 54901 | gagcctgtct aaaactagtt ccactgttca agaaaatgag tgtcaaggct aaatattctt |
| 54961 | ttctgtgttc cccaggcaca gaaatacgag gaacccaata ttcaccacta ggaactcctg |
| 55021 | aaggaccttg tctgctcttc agttctaagc ctgtaaagag gtactttcct agagacagaa |
| 55081 | catcagggtg agagaggctt caggagagcc ccagccattc cctcttctct gggtaaggaa |
| 55141 | tgactgaagc caggagcaca gctaagtctt ctacattggg actatagctt taagcagact |
| 55201 | ttggctggga acccaagggc tcccagccag agtaccatcc tccccaccct ccccctgaaa |
| 55261 | tctgaggcag gccccaccca gtacactggg tgatgctgat ggaaaaMcag atccttgcga |
| 55321 | ttgtagcctg gaaccatttc acagaaggat ctgctgagat ccaccccagc tggatcaggc |
| 55381 | accccccgtc ccccccacaa ccctcactca ataaacaaag aaacaaaagg ccaggccctc |
| 55441 | tgagggcacc tttggagttc ccatctacct gattctctga gaaagaaatt cccccagaat |
| 55501 | ctaaaacaga ggcctcaccg agctggtaag gcaccctctc ttttccctgg tctcaagaag |
| 55561 | taaccaagga cacagaagca gttgcgttat aaacttaagg ccctgcatcc tttttacatt |
| 55621 | tcccaaactg acacaatgca gctcccccac atcaggacaa attcccacat ttacagtaac |
| 55681 | ccctgtttcc agaaaataaa tatcaagata aggaaagtga cactaaagag acacgcagcc |
| 55741 | actctgcctg taccctctct ggaccttaaa gagttaaagc ctagccatga tttcctacca |
| 55801 | gggctgaggc taatgtaaac cacctgctca gacRcaagaa gcaggaccag agggtcaagt |
| 55861 | tgctgagctc tgaagtctgt tctgaaaaag cttcagtttt aagtacagaa caagtgatag |
| 55921 | gccaaaggcc agagggtctg agctgcagca gcactgctct ccttaccccg cccttggcct |
| 55981 | gggtgccagc tcctttgaga ctgcctctta gccacttact gcccatcaag ggaacaccag |
| 56041 | acctacctca agataacacc aaagtgacct ttggtcccag agagttgtta aactataccg |
| 56101 | atctctcagc tcatgggccc ttgcttctgc tgaaccctca ttctcttagt gctaactccc |
| 56161 | actcccagaa tttcagggcc caaagcccac acaagctccc acctcaaagg gtccttccca |
| 56221 | gaaccccacc cccttctcca cctgatagtt ccaaaggcct acagcctcac agctcacaaa |
| 56281 | ggaaatggct gccccatctg tgcgccagtg ctcacttgca aaatttacca ccacttaccc |
| 56341 | tgagacgctg cctgcctgta gcctaagaat tccatgctct gtaccccttt cttagaaaga |
| 56401 | tactccccat ctgagagaca ctcacatcca aagagtgaaa accctgatct tggggctcct |
| 56461 | cctcctccaa ctctRggacg gataggccag gaggcacatg caagcaaaca ccgagatctc |
| 56521 | acctggcagg ttacccagag gggccaagcc accttgccct aggctaataa gctataaaaa |
| 56581 | cagcatagga aatactttgc tggtacatcc cctgcctctg agaagataaa aggcaagagg |
| 56641 | accagtggct ccaagagacg ccatacagag aaaaacaatc tttctcaatt cctggggagt |
| 56701 | cctgggtaga ccgtctggtc caagtccaaa gagtttcctg aggtcggggc aaaccagttt |
| 56761 | ggtccatcta aagggactag cttgggctga agcctcctct tcacacagac cactccatct |
| 56821 | ttgtagccta gagtttggca cagaacccca aagccctgga gaggaacaag ggagggatta |
| 56881 | cagaggaaac caagcttctc tggtttcttc aaaagtggct tacagatgtc tgccccttct |
| 56941 | ccccgttctc tggagggtcc agatcaggat gatggcaaaa gagctgcaat caggaaccag |
| 57001 | acctgcaacc aggcctgcag ccatgtattg tactggggcg cgtacacata caYacacaca |
| 57061 | cacacacaca cacacacaca cacacacaca cacacacaca cacacacaca cacactgacc |
| 57121 | tgggcccccc accactatat tcacacaccc tggtccctat cagctgacaa aatgaacctt |
| 57181 | ttttcactgc ccaccaccgt gcacacaccc acggcctcac gcagaaatgg tgaagccctc |
| 57241 | tcctgagctg tctctacagc tcaacgtgca ggcaaacacg ctttccttct ctgaacaaaa |
| 57301 | acatgctgac atctacgcaa gggggaaaga aagcaaccct ttgctctgaa aggcNccaac |
| 57361 | tcccggatgt tggcaagctc tgctccacag cgcctgtttc tgagtatgtt tacttgctct |
| 57421 | ccctcttctc tccctcccct ttaaggatgg cgcaggaaag ggaggtgagg ggggagaatc |
| 57481 | caccccaccc cctcgtccat gtttgaacaa aaacccagag aaagagtgag ggagaaaaag |
| 57541 | tctcggtgcg ggggggcctt accagactcc taaagccaaa cgtatctaca ggttttaatc |
| 57601 | cgccattagc atttaaatct tgaaaggaga ggaggaaggg ggggccagtg gaaagtaacc |
| 57661 | ccacacacaa aagaaaagtt gttggttttc ttctcagtct gcctggcagg agcgatcKtc |
| 57721 | ctggctgaca gccagaagtg cgtcacagcc aggagtctgc tttctttaaa ggcacagcaa |
| 57781 | gcttccccga tctgggagcg acagcaagca ggggactctc agtgggggtg gggcccagag |
| 57841 | ggagggggtg cactggagag gaggggctgc aaaggggaag gctggaggct tggtccacga |
| 57901 | acaagaaaag caggacgaat gggggcggcg ggttgagggt gaaggcacaa gcaggcaggc |
| 57961 | aaaattactg aactccaaca gcagcaagag ccaaggcaaa ctgtggcata aacacacaaa |
| 58021 | acacatctta cagtggcctg cctgacaaac tgggatcaac cggacactcc cccttcgctc |
| 58081 | tgttttccac ccagagttta aggcagcagt aataatctgt ccatctctct ggacagcaat |
| 58141 | ggaagggtag gacaagggct gtcctgataa ttagatcagc ccctgcctgg cggaccgcag |
| 58201 | ggcaaggagg aagacccggg aacagaggct ttgaagaaca agcaacatgc aggccctctg |
| 58261 | atcgggggca ccttcctgcc ggccccaggg caggctgtgt ctttaagtac aaccatgcca |
| 58321 | ctcacacaca aagcgccctg ccaccttcca tgggcaccgc ctgccctgct ctgggacacg |
| 58381 | gccacccact aaatccattc cccgagggaa tctctccagc ccataccttc tgcgtggcaa |
| 58441 | ggccaggatg ccgtgctgtg cccgctcctc cccctccgcc acccaaacaa gaagcccagg |
| 58501 | cagattctgg ccctggagta caggcctgcg ctgtcctgtg ctgcgtgcta ttgtcggctg |
| 58561 | tggaatggcc gccaaaatcc cagctgtgct gccagaacct aatcccctgt tccgctgagc |
| 58621 | atttatgggg aggggctgag gggccaaggc acgagaccga gtacgccagg cactggctgc |
| 58681 | agcccttaac agacgcaggc acSgggactt gtagactctg cttctcctac atggctgaaa |
| 58741 | acaaaagatg tctgactctt agaagacaaa aYcacagcat ttccccccac tgaatagcga |
| 58801 | acttcaccaa ggctgaaagg gaagcccacc agatggtcaa caggcacccc ctaatcttat |
| 58861 | gccacaaaca tttactgagc acctactatg tgtaaggcac tcctatagga aactgtagga |
| 58921 | agtatacaaa gatgaattaa gcccagcatc cgcactgagg taggttgtaa ttcacaggag |
| 58981 | ataagaaata tgacttgaag attaatttca aactggtgtg ctaacttcca taaaagctgt |
| 59041 | aaagaaaaat tctaaccagt ttagaatagg gaaggattac gaaaatgaag tgccatttta |
| 59101 | atttggacct tgaaacacac aggtagcatt tccccagatR gaaacagaga ggtgtgggga |
| 59161 | ggaagagaaa gcagagagga ggcatggaag cagtgggaga aagggcttga agagtgaggt |
| 59221 | ggggtcagag agctaggaga atcttgaacc tcaaaccaag gaatcaaaga cttgtaagtt |
| 59281 | tgtttgttat taacacagag ctgtggtatg acaaaaactg tgccccaagt ttattctgat |
| 59341 | atatacctga atgagactag cagcaatgag gtgagtcagg aagctgcaca gtcaggagac |
| 59401 | aggtaatgag ggcacacaag agggtaaggt aagaaagaat ggaaaggggS cttggcacga |
| 59461 | Yggctcacac ctgtaatccc agcactttgg gtggaccact gaagtccgag agttggagac |
| 59521 | cagccggggc aacataggga gatcccatct ctacaaaaaa tttcatgaat aaaaaattag |
| 59581 | ccgggtgtgg tggcccgcac ctacagttcc agctacttgg gaggctgagg tgggaggatc |
| 59641 | acttgaaccc aggtggaagc tgcagtgaga gacattgtgg agacagggta aagaactgat |
| 59701 | aggaggcaga gagaggaact gtacagggga ggaaagtatg agggatgata tgcagaatta |
| 59761 | aactgaggag atgacaggat gtcagaacaa gagagaaaac agagtataat aaatagtagc |
| 59821 | tggtggggca cggtggctca cgcctgtaat cccagcactt tgagaggcca gcctgggcaa |
| 59881 | catggcgaaa ccctgYttct accaaaaaat acaaaaaacc acacaaaaaa acaaaaacaa |
| 59941 | aaaattagcc aggcggagtt cttttttttt tttttttttt ttttaaagca gggtctcact |
| 60001 | ctgtcaccca ggctggaggg cagtggcgag atcatagttc actgtaacct caaacttctg |
| 60061 | ggatcaagtg atccatctgc ctcagcctcc caagcagtgg gagtacaggt gtgtgccacc |
| 60121 | atgcctggct aatttttttt tttttttttt tctgtaRaga caggctctca ctatgttgcc |
| 60181 | taggcacagc attattttaa attcttctct aaaaattcta tgcttctccc aaagccctgg |
| 60241 | gatacaggca tgaaccacca cacctggccc acagttgtct ttgtcttttt ctactaggag |
| 60301 | ctatactata gaggtcacaa tcattggcta cagtttataa tatataggct aaaatgtttt |
| 60361 | acatataaRa taaccagtaa aacttcatga tttaRaaaca aattagtgtc ttttttagtg |
| 60421 | tcaRaRtccc agctactcag gaggctaggg tgggaRaatt ccttgaactc ggaatgagca |
| 60481 | gagatcatgc cactgcactc cagcctgggt gccagagcaa gactctgtct caataaatac |
| 60541 | ataaataaat ggacagatag ataaacagag agacagaaag acatatagta gctaatgctt |
| 60601 | actgagtgca tactatgtac Rcgacactat tctactgacc taatatgtac gaactcatta |
| 60661 | atcttaaaaa ctcccttaat gaagtaggta ccattatcac ccaactttag agaggaggat |
| 60721 | attgagccac aagattaaat aatttgccaa gatcacatag caagtaaatg gcagaagtct |
| 60781 | ctaccacatg ctgctaacta tttgacattg gcaagaaact tctgaacttt agtatcccta |
| 60841 | tttgtaaaac aagaataaag acacctgtta taaagcaatg aaaattaatt caataacacc |
| 60901 | tataaggtgt ccaacacact ggctagcatg taaggcatag gatgggtgtt tcctgcagac |
| 60961 | taattcctac ttggcatttt ggtcccttgt caccaaggtg tccaagcaga agttggctca |
| 61021 | ccaattgttt gggaagccag agagggtatt ctgcctggag caggagactg gatcctctgt |
| 61081 | ctgtctgcct tcttcccatc agtctgcctc tctagatttt actttagggt ccagctcaaa |
| 61141 | tatttaatat atttgattta atattcctaa ctcctcaaat atttaacagt taacaggggt |
| 61201 | tactttNgSg ggggggagga gttagattgt agggaacatt cacttgggga cttYctatgc |
| 61261 | tgtatgtttg cttttttttt tttttttttg agatggagtc ttgccctgtc acccaggctg |
| 61321 | gagtacaatg gcatgatctc agctcaatgc aacctctgtc tcctgggttc aaatgattct |
| 61381 | cctgccacag cctaccaagt agctgagatt acaggcacct gccaccatgc ccagctgatt |
| 61441 | tttttatttt tagtagagac agggtttgac catgttggcc aggcttgtct cgaactcctg |
| 61501 | acctcgtgat ccgcctgcct caggctccca agtgctggat tacaggtgtg agccactgtg |
| 61561 | cctggccttg catttatttt taataatctt gtattacact ggtgataaga aaaatgtaac |
| 61621 | tacctcctac tcttggcact tatgtgtata tgcatgaaaa tcttgtcttc acatctagaa |
| 61681 | tggaaattcc tcaagggcat aaccagttcc acctctctct ctctgtgatc ccccataaca |
| 61741 | cctagcacac tagggctcct taaatctgct tctccaaact acatctggat gtcaggttta |
| 61801 | caaattcaac tttctggcca ccagagaatg actgtggtgg gtggttggga tgaSgtgggg |
| 61861 | gtgggggtca gaccaaggct cattgctgta ggctccccat cacctgctgc ttcttccagg |
| 61921 | ccctgtaagc tattcccact gtatcatttg tagtctgtag ttagcaatca ctattagtag |
| 61981 | aaatggaagt tcttatccaa aggagcacca tgaaagcaaa ggcaaagcag ataccaaaac |
| 62041 | ttttacagaa aagcaaagtg gcaaacataa ttgtttgggt ttttgttgtt tttgttttct |
| 62101 | tgagacagtc tcactctgtc acccaggctt gagtgcaagt ggcacaatca tggctcactg |
| 62161 | cagcctcagc ctcctgggct caaacaatcc tcccacctca gcctccggag tagttgggac |
| 62221 | cacaggtgca tgccaccacg cctgactaat ttttgtattt ttttgtagag acagaatttc |
| 62281 | accatgttgc ccaggctggt ctcgaactcc tgggctcaag cgatctgcct gcttcagcct |
| 62341 | cccaaagtgc taggattaca ggcatgagcc atcacaggcc tggccaaact taatttaaag |
| 62401 | actggcatta tcaatgctcc agaaaatgta taaggtgaaa ataggaggga ggcctgctaa |
| 62461 | aactgccctg gccacatatg gcaatgcatc agccctgaag cttcctagcc cagatagaat |
| 62521 | tgatattgtc tcaaaagcgt acaagaggtc tccccagcct ggccccttgc tctaaactca |
| 62581 | aggcaacagc ttccagctgt gcagcttccg agaggctgaa ccttccccta gaacccctct |
| 62641 | gcaaagctgc caagtttctg ggccagctct tcaggacagc ccaaggagcc tggccagttt |
| 62701 | cgccaggctt tccctcctct tcctccctcc tgtggcagca gcagaatttc cacagcccct |
| 62761 | ctcacagcct agacaggcag caggcggcca catggctcct gcctgattct tactgtcctc |
| 62821 | agaagagcgg gaaaggagtg gtaaaacctt tctagaaaaa gagaagggcc gggcacggtg |
| 62881 | gctcacgcct gtaatcccag cactttggga ggccgaggca ggcggatcac ctgaggtcag |
| 62941 | gagttcgaga ccagcctgac caacatggag aaaccccatc tctactaaaa atacaaaatt |
| 63001 | agctgggtgt ggtggcgcat gcctgtaatc ccagctattc gggaggctga ggcaggagaa |
| 63061 | ttgcttgaat cctggaggcg gaggttgtgg tgagccgaga tcRtgccatt gcactacacc |
| 63121 | ctgggtaaca agggtgaaac tccatctcaa aaaaagaaga aaaagagaag aaagaaaggg |
| 63181 | gtggagagcg gggagaggga gagagagaga gagagaaagg aacaaaggaa ggaaaagaga |
| 63241 | gaagaaagga aggaaagaaa ttactttaaa ttcagtaggg agggctgggc acagtggctc |
| 63301 | atgccttaat cccagaactt tgggaggtcg aggtgggcag attgtttgag ctcaagagtt |
| 63361 | caagaccaac ctatccaacg tgacaaaatc ccatgtctac taaaaataca aaaattagct |
| 63421 | gagtgtggtg gcaggagcct gtaatctcag ctacttggga ggctgaggca agagaatcac |
| 63481 | ttgaatccgg gaggcggagg ttgcagtgag ctgagattgc gccattgcac tccagtctgg |
| 63541 | gtgacagagt gggactccat tgcaaaaaaa gagaaaaaaa aagaatttta aattaaaata |
| 63601 | aataaataaa tttcagtaga gttcagatag aggttgaagc ggggtggtga ctggttagga |
| 63661 | gcacaaaggt tatttcaaag ggtcaggtga tcctcctgcc tcagcctcct gagtagctgg |
| 63721 | gactacatgc acatgccatc gcatcaggat ttacattatc atttttgaaa gtttaaacaa |
| 63781 | aaaaagactg gagctgggtg tggtggctca tgcccgtaat cccagaactt tgggaggcca |
| 63841 | aggtgggagg ataatttgag ctcaggagtt caaattcctc aggagttcaa gaccagcctg |
| 63901 | ggtaacatag cgagatcccc atctctacaa aaaataaaag aattagccag acatggtggt |
| 63961 | gtgtgcctgt agtcctagct actcaggagg ctgaggtggg aggattgctt gagtccagga |
| 64021 | gtttgaggtt atagtgagct atgatcatgc cactgcactc tagcctgggc aacagagcaa |
| 64081 | gacacgtctc aaaaaaaaac aaaaacaaaa acaaataagt actaggagag ttcaagtccc |
| 64141 | cagtcatccc catgggatga ttctcacaca aataattagc ctggcactca aggccctcca |
| 64201 | tgacctgcag cttctagagt tctagcttct agagtcgtta ttcccttacc caaaccctcc |
| 64261 | actctgccag gcaggtctat aaagttacag tttgtcctga gaattagaca agatgaccgc |
| 64321 | aggggacagg ctctggatta gctacgaatt acaacagtaa cagcaacgat aatgatagtc |
| 64381 | tttactatgc caggatgtaa gtgacagaga aaagactaga aggcattgta ttgggcagtt |
| 64441 | ttgtttattt tactttattt ttttaataaa attaaaaaat atatggaact cttcacaaat |
| 64501 | ttgcatgtca tcctcgcaca ggggccatgc taatctctgt atcgttccta ttttattttg |
| 64561 | ttccaatttt agtatacatg ctgccgaacc aagcactggg cagttttaaa aatgaagaaa |
| 64621 | tctgaggctc aggaattagg tgacYtacgt gatctagtca tgtaagtagt aagttgtcta |
| 64681 | ccttgtccct atattattct tttttgtttt tttctttcaa gatggagtct cgctctgtcg |
| 64741 | cccaggttgg ggtgcagtgc tgcgatctcc gctcacYgca acctctgcct cccgggttca |
| 64801 | tgtgattctt gtacctcagc ctcccgagta gctgggataa taggcatgag ccactgcacc |
| 64861 | cagccagcct atattattgt ctacagctgt taaataggca atcattgagt gaatgaataa |
| 64921 | aagctagcac attctcagca aatcaaagaa gccgtgtgct tttcctcatg aaaatcaacN |
| 64981 | ttttttNatt ttttgagaca gagtttgctt tgtcgcccag gctggagtac aatggcacga |
| 65041 | cctcagctca ctgcaacctc cacctgccag gttcaagcga ttctcctgcc tcagcctccc |
| 65101 | gagtagctgg gattacaggc gctcgccaac atgccggtct aatttttttt atttttagta |
| 65161 | gagatggcgt ttcaccatgt tggccaggct gggcttgaac tccttacctc aggtgatcca |
| 65221 | cctgcctggg cctcccaaag tgctgggatt acaggagtga gccaccgtgc caggccgaaa |
| 65281 | atcagcttct taatcaaaat aatcaaacag ctccaatttc aaaattaatc actaaagaat |
| 65341 | ttttttctga ggatccgaga gctttacaaa cactRaaggg ccaaaactcc tctaaaaaca |
| 65401 | gcaagcacca agaaaagtct tgtactagga gggatgttgg cagtgtctca agggttagag |
| 65461 | ggggaggctg gaaggaaatg ctggtgctaa agggaaggac tgctgtttca ggagcctgac |
| 65521 | cttgagctta tttaccatgg cctcattgtc cctctccaga Rgataatggc tgattaatga |
| 65581 | ggcagtgttg gtaagtctga ggcttcctgg agagaaaagc agagggcaaa tcaaaaccca |
| 65641 | gagaggattc attataacat ccaacaggcc cactcagttc tctctcaaga aggcctttct |
| 65701 | acaaaacagc taagaacctc tccaccctgt ttcactgtca cagaggagta aaatggctct |
| 65761 | gtccctacag gcaacatatc aacatatcta gcacaatgtt tgctcacaaa cacagaacta |
| 65821 | attataagaa atgtgaaaga acctgaaacg cagggggaat actcttactg gagacagcct |
| 65881 | ccatagtggt agaaaacata gtagatctgg tgttgaaagc ccctggaggg gtgatgggtc |
| 65941 | acttactagc tatgtggctt aatgcaagtc acagaggcaa attactgaga accaagctag |
| 66001 | ggtcggatca gctctaaagt accttttcag ggccaggcac agttgctcat gcttgtaatc |
| 66061 | ccggcacttt tggaggccaa gatgggagga tcacttgagc caaggaattt gagaccagcg |
| 66121 | tgggcaacat agtgagaacc tgtctaaaaa aaaaaataat aataatatat atatatatat |
| 66181 | atatatagtg tgtgtgtgtg tatatatata tatatatttg cctattcatc tatctataga |
| 66241 | tagataaata aatagatata tatcaaaaaa agaaaattgg ctaggcatgg cggttatgtg |
| 66301 | tctgtagtcc tggcaacttg ggaggctgag gtgggaggat cacttgagcc caggagtttg |
| 66361 | gggttacagt gagctatgat catgccactg cactctagcc tgggcaacag agtgagaccc |
| 66421 | tgtctctaaa aaaataaaat aaagtacctt tccacttcca aaaattccca tgattctttc |
| 66481 | cacaggatac cattctcaac aacaaatctt gtagtagaac ttgtctcctc agaatctggt |
| 66541 | caaaacatca ctagttaggg tgttctttca aaaaaaatag caaatctcct agaaacaggc |
| 66601 | acattgtgtg ttaccctatt aggacccatc cccaaagcga ggcacatgtg ggtgcttcat |
| 66661 | taatactctg ttgaaaggag gaggtccttg gaaactggta gtaccaactt agcatggcat |
| 66721 | acaatgaaag gtgattggag ccacactcga ggaactacaa ggcagctttt cataaataaa |
| 66781 | agccacaaaa tgcaatttat aactacatct gtctaaacag actaaagaaa agtagctttc |
| 66841 | tatacctagg tcttcacccc ttctcctccc cacctctcta ctttatcatc cctgtcctac |
| 66901 | acagcactga gacccgtctt tctggttcca ggtccacttt tcctttttcc acaccagaat |
| 66961 | gcctttaatc actgatagaa atgaacaaac atgaatatga gttctaaaca ggaagacaga |
| 67021 | tagcctcaag aaccaacttg tcaatatgtt actaataagt gaaataagtg ggaatggtgt |
| 67081 | ctaacatggg catgataaaa taaaacccta aaatacatac tgtgctaggg atttgaacaa |
| 67141 | acttcaggat ttttaaggaa ggctgtgtct cacatctcaa ttaatcttcc caaaagaacc |
| 67201 | ctaagaggca aggtttattc caattttata aattaggaac gtaaggctca gtaaaaagaa |
| 67261 | aatacctgcc tcaaggtcga ctggtaaggc tgcagagcca gaactcaaag agatgtcttc |
| 67321 | taaatccagt gctctctcca gcacatctca gctacatcct ctatctttct catcagtata |
| 67381 | ggtcttcatg tatacaaagc atttctgcat tcattgttca cactgcttct gccacagctc |
| 67441 | atgaaaagga caatgcagat gtctaaatgg caccagatct atacccacct tttttttttt |
| 67501 | tttttttttg agacagggtc ttgctctgtc acccaggctg gagtgcagtg gcaYaatcat |
| 67561 | agctcgctgc aacctcaaac ttctgggctc aagtgatcct cccgcctcag ccttacaagt |
| 67621 | agctgctgct acaacaggca cgYgccacca cgcctggcta tttttaattt ttagtagcga |
| 67681 | tgaagtctcg ctatgttgcc taggctggtc tcRaacacct gggctcaagc gatctgcctg |
| 67741 | cattgacctc ccaaagtgtt ggaattaaac ggtgtgagcc accgtgctgt cctatccaca |
| 67801 | cttttaaaga tgctaataaa taacgtgatc accatcagag aaaataaatc ctacgtatct |
| 67861 | ggcatcactg agaagtacaa aatttcacac aagcaatttt cctactaact gaaatttatc |
| 67921 | ccttttaagg caaagctctt tctttatatc aaaacatatt ctccaaaata cataagatat |
| 67981 | gttactcttt acttagaatt aatgaaattt aatcagggat attcgactat aaacatgata |
| 68041 | taaacatcct acatactgtg ctaggaattc aaagattttt tttttttttt ttgagacaag |
| 68101 | gtctcactct gctgcccagg ctggagtgca gtggtgcaat ctcagctcac tgcaacctcc |
| 68161 | acctcccagg ctcaagtgat cctccctacc tcagcctccc aagtagctgg ggctaccggt |
| 68221 | gtgcaggacc atgcccagct agttttgtac tttttgtaga gatggagttt tgccatttgc |
| 68281 | ccaagctggt ctcaaactct tgagctcaag cgatcagccc accttggcct cccaaagtgc |
| 68341 | tgggattaca agtgtgagcc actgtgcctg gcccaagggt aaatctttaa acttgagttt |
| 68401 | ataatcttgt agaggaagag acacatctgt aaccataata tgaagccaaa gaacatgggc |
| 68461 | ttggggtgtg ctgaaaggaa ggtaagataa cacaagcaag gctagtgggg accaacaacc |
| 68521 | cagggtgact tactgaagag aagtgggagg cactgaaaag tcctgggcag gagaatggta |
| 68581 | tactacttgt gctttttgaa aagatgactc tagcacaaca gtgaggccag aggcagaaaa |
| 68641 | tggacttagg tggcagcgac agaggtagag agaaaactgc actgccaggg caggggcagt |
| 68701 | aggaactaaa acaggtggct ctgaaaggca ttacagacgt agagacaatg ctcttagcat |
| 68761 | ataggggcgt gggaaaggtg gggagctaaa gagaacacta agattcttgc tttcatggaa |
| 68821 | agatttcaga aYgagaaaat aagagtaggt actggtttaa aggagcgtga gggactaact |
| 68881 | caggatggga tgatggggaa tgctttatag aggaagtgac atttaagcta gatattaaaa |
| 68941 | tatgagcagg ataagtagag tttcaccaaa aagtaaaaaa gcattctagg cagagaaaac |
| 69001 | agcacatgca aagacagacc atattacagt tgaacaatgg catgcagttt tagggaggtg |
| 69061 | catcagatga taagaagggt aataagtaag gccaggggcg gtggctcacg cctgtaatcc |
| 69121 | cagcactttg ggaggccaag gcaggtggat catgaggtca ggagattgag accatcctgg |
| 69181 | ccaacatggt gaaaccccgt ctctactaaa aacacaaaaa ttagctgggt gtggtcacat |
| 69241 | gcctgtaatc ccagctactc aggaggctga ggcaggagaa tcatttgaac ccaggaggtg |
| 69301 | gaggttgcag tgagccgaga tcatgccact gcactccagt ctggtgtgct ccagtcagag |
| 69361 | caagactcca tctcaaaaaa aaaaacaagg gtaagtaata ttaaattgtg aggcacttgc |
| 69421 | ctcatactaa gaaattgtag gtgatgagaa gacaatcaag gtttctaaag gaagagggta |
| 69481 | aaggggagca gcatgatcag ggctatgttt cagaagggaa actgtataaa gtacagactg |
| 69541 | gaatggtaag acacaggagg aaggcaatag agcataatgg ctaagattac aaggtctgaa |
| 69601 | cttaagacaa cctaatacaa attctgggtt tatagcttaa tagctctgca accttgggca |
| 69661 | agttatttac tctctctgtg ccctcatttt ccccagctgt aaaatgggaa taataatagt |
| 69721 | actttatcag gcacaagcca ccacacctgg ctattttatt tttaattttt gtagagacag |
| 69781 | gatcttgcta tgttgtccaa gctggtctcg aactcctcaa ctaaagtgaa gctcccacct |
| 69841 | tgtcctccca gagcactggg ataacaggta tgagccactg tgcccagccc atagttccta |
| 69901 | attacgcctt tttgtatttt attaatttat ctttctctta ctgccttaag ttccatgaaa |
| 69961 | tcacaaactg tgtgctcacc gctgaattct tagaaatcag cacaatggct ggcacagcat |
| 70021 | gagtgcttga taaatacttc ttgaatgaat gaaaggttcc aaactatata ttttaaatga |
| 70081 | actctattta aatgtaacct ttatatagaa ccccaggaca cacagaagaa gacagtttga |
| 70141 | agtaagggat atttggagac ttgttccctc tgcttccccc tggKtcttaa gtcagttcta |
| 70201 | tagttctgta aagctttaca gaatgcagtg caaaaaccat tgtctgttac aagcaagaag |
| 70261 | taatgtatgt ctgaagtaag gcaatgacaa taaagatgca acaatagctt tgagaactat |
| 70321 | ttatgacagg ccctgacaac caattacagt aggccctctg tatccatggg ttccacatgt |
| 70381 | gtggattcaa ccaacctgag atgaaaaaca ttttRgggaa aaaaaagcat ctgtactgaa |
| 70441 | gatgtataaa cttttttttc ttgtcattat tctaaacaat acaatgtaac aactatttac |
| 70501 | atagtattta cattgcatta tataagtaat ccaaggatga tttaaactat acgaaggacg |
| 70561 | tgtgaaggtt atatgctaat attataccat tttatataga gacttgagca tccaaggatt |
| 70621 | ttggtatctg tgtgaagtcc tggaaccaat ccccacagat gctgagggac aactgtataa |
| 70681 | atgaagaatg agaaaaggaa aattcttgaa gcacctctaa atttcctagc ttgagtgaac |
| 70741 | aaataaatga acatgtgtgc caaaaagcaa gataagaatg taagagtggc tgggcacggt |
| 70801 | ggctcacgtt tgtaatccca gcactttggc aggtcaaggc aggtggatcg cttgaggcca |
| 70861 | ggagttcgag accagcctgg ccaacatggt gaaaccctgt ctccactaaa aatacaaaaa |
| 70921 | aattagccgg gcacggtggc aggcacctgt aatcccagct acgagggagg ctgaggcagg |
| 70981 | agaattgctc aaacccagga ggtggaggtt gcagttagcc aagatcacac aattgcaatc |
| 71041 | caccctggga aacaagagca aaactccatc tcaaaaaaaa aaaaaatgta agagtaacaa |
| 71101 | taacaatttc cattcatttg aacatagtat cacccctatc aagcaaatac tattaatacc |
| 71161 | ctcaagttat agaataaatc tttcacgatg ttaagcaact gtcatgatct tcattagcaa |
| 71221 | agatcatgag gctaataagt agcagaatta agtttcaaat ccaagactgt cccactccaa |
| 71281 | gcccaaaatc taaaccgtct tgctatattg gcaagcagtt tgagagttag gtcttagacg |
| 71341 | tgagatatct actggatata cagttagaaa taatcagtaa ctaatgaaaa atatgatttg |
| 71401 | gggttagaga taaggatctg ggctccattt ggaagactga ataaattcag aagagtgtct |
| 71461 | atgagaagat catgcttgct tgtaaggcct gtaaatgggt gatgtacgca tttgcgattg |
| 71521 | aagtttgttg atttaccaag gtctctgcct tgcatagatc tagtgggcat cRgcattaag |
| 71581 | agaggaagta aaaaacttta ggagaaattc cagatctctg gggattcctg ttaggacctt |
| 71641 | tatattattt ttgttttggg agaatttaac agaaagacag tgacaccaat gtcaacaaaa |
| 71701 | aaaaatgaga cagcaaaggt gataaagaga aYagatttgg ggatcatcat tttctcttct |
| 71761 | gcttgagatt ccacagctct caacaaaaat gactattatt tgtgtatggc tctttacaac |
| 71821 | taacaaagtg tttcccattt acaaggtatt tcctgtttac caataatgtc agatgaacct |
| 71881 | tacaacagct gtttaaggtg agggcattat tattttcact ttaccaaaga ggaatgaagt |
| 71941 | ccagaaaagt taagtttacc ttgtctaagg tcatacagct aaaaagcaac cgggccacga |
| 72001 | tgtaaccacg gttttctggc tcctaatctg gggctctttc cagaagactc acctgagata |
| 72061 | acatgagcca ctcacttatg aaccaagagt catcatccct atgtatagtt tcactgtgtt |
| 72121 | taaaaataaa acaatagggg gatagtgact gggaaggggc taatggtatg ctggtaatgt |
| 72181 | tctgtttctt gatgtaggtg ctagtaacac aggtgtgttc accttgtgga aagtccttga |
| 72241 | gctgtaaact ttgtgtactt ttctgtatat atgtcatact gtaattatta aataccaggc |
| 72301 | aagccaggtc aggaggcttg cacttgtaat cccaatggct caggaggctg agatgggagg |
| 72361 | atcacttgag cccaggaatt caagaccagc ctgggtgaca aagcaagacc cccatctctc |
| 72421 | aaaaaaaaaa aaaattcagc tgggcgcgga agcttacacc tgtaatctta gcactttgag |
| 72481 | aggccgaggc agacagatta cctgaggcca ggagttcgag atcagcctga ccaacatggt |
| 72541 | aaaaccacat ctctactaaa aatacacata cacacacaca cacacacaca cacacacaca |
| 72601 | caattagcca ggtgtggtgg tgcacgccta taatcccagc tactcagaag gctgaggcac |
| 72661 | aagaattgct tgaacctgga aggtggaggc tgcagtgagc tgagatcata ccagttcact |
| 72721 | ccagcctggg tgacagagtg agactctgtc tcaagagaaa caacaacaaa taaaaaaaaa |
| 72781 | ttagccaggt atggtggcat gcctgaagtc tcagctactc aggaggctRa ggcaggagga |
| 72841 | tcacttgagc tcaggagttt gaggcttcag tgagctacga ttgtgccact gtaccccagc |
| 72901 | ctgagtgacg cagcaagatc ccatctctct tttttttttt ttgagttgga gttttactct |
| 72961 | tgttgcccag gctagagtgc agtggtgtga tcttggctca ttgcaacctc cgccttccag |
| 73021 | tttcaagcga ttctcctgcc tcaacctgcc aagtagctgg gattccaggt gtctaccacc |
| 73081 | acccccagct aatttttgta tttttagtag ggacagggtt ttactatgtt ggccaggctg |
| 73141 | gtcttgaact cctSaccttg tgatccgcct gcctcagcct cccaaagtgc tgggattact |
| 73201 | ggcatgagcc atcgcgcctg Rccaagatcc catctcttta aaaaaaaaaa aaaaaaaaaa |
| 73261 | aaaaggcaag ccaaactgat aatcaaaact caggaacact aagttggatg cgtcacattt |
| 73321 | tttactcaat ttctcaaaca gtagctacat ataggacatc taccagttat ataaggatgt |
| 73381 | acaaaattct tgcacactca aataacatat ccacccaatt gtcccaatcc agactgtctt |
| 73441 | agtgatgtga aagaatctga caggactgga agtaacagaa gcccaggaaa gggcggagat |
| 73501 | ttttgttaat gaaaacaaaa atggcattga aaatggaact atcctacatc atcatcaatg |
| 73561 | atgagatatc tcaatttcag agataacaaa aatgtgaaaa aaaaatggcc agtgacaatt |
| 73621 | ggaagatact atcaattgta aaatacaccc caatttcagt gttaaaatat taaaaaagca |
| 73681 | aaaaaaaaaa aaagaaagaa agcattttaa aacaaaggaa atatattcta atctcaaaag |
| 73741 | gtagctcaat tgtgggataa cagatttgtg aagcacatct ccttgtacaa gctctttgtt |
| 73801 | ttagagataa ggacactaaa gccttcattt aagttattaa ataaccttac taaattaaag |
| 73861 | ctcacacatc agaaagttga taactacaat tacaaagctg gttccttcaa tgctcgttct |
| 73921 | cctgccacta cccagatgcc tcctttaaat ccaaaagaag atttacatat taattccata |
| 73981 | ttaaaactct attttgactt tcatatttct ttacttcatt aactcactaa aaaataataa |
| 74041 | aagttaacct ttaaaaatca attaaagctg ggcgaggtgg ctcatgccta taatcccagc |
| 74101 | actttggaag gctgaggcag gtggatcact tgaggtcagg agttccagac cagcctggcc |
| 74161 | aacatggtga aaccccatct ctactaaaaa tacaaaaatc agttgggcat ggtggcaggc |
| 74221 | gcctgtattc ccagctactt gggaggctga ggcatgagaa ttgcttgaac ccgggaggtg |
| 74281 | gaggttgcag tgagccaaga ttgcaccact gtactccagc ctaagaaaca gagcgagaca |
| 74341 | ccRtctcaaa aaaaaacaaa aaaaaacaaa aaacaaaaaa acaaaaaact aagctgggtg |
| 74401 | cggtggctca cgcctgtaat cccagcactt tgggaggctg aggcaggtgg atcgcctgag |
| 74461 | gKcaggagtt Ygagaacagc ctggccaaca tagtgaaacc ctgtctctat taaaaaaaaa |
| 74521 | aaaaaatagc taggcgtggt agcaggcgcc tgtaatccca gctactaggg aggctgaggc |
| 74581 | agaagaatca cttgaaccca ggaggtggag gttgcagtga gccaagattg tgccactgca |
| 74641 | ttacagcctg ggcaacaaga gcaaaactac atctcaaaaa aaaacataat aataattaat |
| 74701 | taagatgaaa cgatgtttag gaaacttcat ttcttgctta tacaagtttg ttttccttgt |
| 74761 | tcttcaaaac cagtgaagca atacaaagtt agataaaaga caaaattagt aatctcccag |
| 74821 | cccctttcca ttcctctcca aggcctccta caagatgaga aatggtaaca tatttttgtg |
| 74881 | tcccccaaaa acataaatac aaatatattg gaaagggttt tgttgttctc tgtttttaga |
| 74941 | aaactaggtt taaactttac acattgctct gcaacttatc tcatctaaca atttatcaac |
| 75001 | atctccccaa gcatcaataa ctaaaattct aactcatact tttaaaaagc tacataaaag |
| 75061 | tccatataag ggatatccat aacgtgttca catatttttc tattgaggga atggctattt |
| 75121 | tcagtgttac catttcaaac aatgctgtaa tgagtatcct tgacgtattt caagttaaag |
| 75181 | aaaaaaaact atttctacac acatactttc cttgttgtcc catgttttga ttactcctgg |
| 75241 | tctcatcatc atccctacct cccttgctca gagaatcatc ctcagggtcc agtgtgatga |
| 75301 | cagagagcaa agtaacttgg agaatgtcaa gacaagacat gtgatagtta ctgttggtaa |
| 75361 | attggaaatg gcctgtcttg gctgcctctc tcagtcactt gcacatgcta tccccctcct |
| 75421 | ccccttacat gtgctccctg ttctggtctc agcagctgct tccaactcaa ctgatgtgag |
| 75481 | cacagctagc tacYttgtca cactcctaaY cctggcaagt gcagacaggc ccaggagctc |
| 75541 | tccaaacaat tggaaaggag aaggaataca ttatcactgt gaaataatat aatacggagg |
| 75601 | tatctagagt aaaatgtaaa actccctctt cattaagcct caacccacat ttgccagtga |
| 75661 | cactccccag ctcagaagtg accactgtta gaagtctgtt gtacaacctt ttggattttt |
| 75721 | cctaagcatg tacacataca ggtatatatt cacatacagg tatgtgtgtt tggtctacat |
| 75781 | atctgtaatc ataccatata atatgttctg caacttgccc ttttcaYgta Maaatttatt |
| 75841 | tgacatgttt ccataccagc aaaaaacaga ttcctcacat ttttaattaa ttttttttga |
| 75901 | gacaaagtct cactctgtca cccaggctgg agtgcagtgg caccatcatg gctcactgca |
| 75961 | acctcaacca cctgggctca ggtgatcctc ccacctcagc ctcctggtag ctgggactaa |
| 76021 | aggtgcRcac caccataccc agctaattta tttgtagaga tgggatttca ccatgttgcc |
| 76081 | caaactgttc tcaaactcct aggctcaaac aatccgccca ccttgtcctc ccaaagtgct |
| 76141 | gggattacag gtgtgagctR tcatggctgg caattcctta aacttttaaa agcttRaaaa |
| 76201 | aatcctatgt aagaatgtgc cacaacttta ttttcccatt ttgctacatc tggacattta |
| 76261 | ggttgttcac agtgttttgc atgtcaaaga atgctgtatt gataacactg gcagcacatc |
| 76321 | tctaatttta cacacaagct aatatttctc taggatatat accaataatt agaattaWtg |
| 76381 | ggttgaaaag cttgtgtatt ttaaatgtca acagatattc ccaaattacc atctaaaact |
| 76441 | gtgtactagt ttatagtctc accaacagag gataaaaatt cccatctaca cccttctacc |
| 76501 | aatatatttt atcaatatat aagatagaat atattattgt tttaatttat atcttatttc |
| 76561 | cagaaaagtt aaacatctcc tcacatatgc aactgttcat tcttatttct tatgaaaggt |
| 76621 | attaccattt tgattttaaa catgagggtt cagagatgct agagtgacac aaccagtaag |
| 76681 | tccagacatc aaagctgttt ttttaacaat ttcccatcaa aactaggcct ctttctgtca |
| 76741 | agtgtaattc cccctaccag cagccagcaa actatggcct ataagttagc agcccatatt |
| 76801 | ttgtaaatca ggttttacta aaacgtagcc acgcccattc atttaagtat tgtctatggc |
| 76861 | tgcttttgtt ctccaatggc agaggtgagt agttacaacc acatggtctg caaagcctaa |
| 76921 | aatatttact actggggtct aaacagaaac aataacttgc ctaagcacag tgactcacac |
| 76981 | ctgtaatccc agtgctttgg gaggctgaag gtggtggact gcttgctgtt tgagcccagg |
| 77041 | agttcgagac caacctgggc aacatggcga gattccatct ctacaaaaat tagaaaaata |
| 77101 | gccagttgtg gtggcatgca cctgtaatcc cagatactca ggaggctgag gtgggaggac |
| 77161 | tgcttgagcc tgggaggcca aggccgtggt gaactgccat tgtgccacca cattccagcc |
| 77221 | tggaagacag agtgagactc catgtcgaaa aaacaaaacg cacacacaca aaaaaaacaa |
| 77281 | gagttaaaaa ctaaagaaaa gctcaRgtct gggcacagtg gctcatgcct gcaatcccag |
| 77341 | tacttaggaa ggcttagcga gaccccatct ctacaaaaaa taaaaaaaat taggctaggc |
| 77401 | acggtagccc acacctgtaa tcccagcatt ttgggaggcc gaggcgggcg gatcacgagg |
| 77461 | tcaggagaat cgagactgtc ctggccaaca tggtgaaacc ccatctctac taaaaataca |
| 77521 | aaaattagct ggccgtggtg gcgtgtgcct gtaatcccag ctactcagga ggctgaggca |
| 77581 | ggagatcact tgaaccaagg agttggaggc tgcagtgagc cgagatcgtg ccactgcact |
| 77641 | ccagcctggc gacagagcaa gactgtctaa aaaaatttaa aaaattaaaa attagccagg |
| 77701 | cacagtgctg tgtgcctgta gttccagcta cttgggaggc tgaggcagga ggaccccttg |
| 77761 | aaccaaggag ttcgaggttg cagtgagcta tgatcacacc acggcactcc aaaatctggg |
| 77821 | tgatagagca agaccctgtc tcaaaacaaa aaaagaaaag cttctcagtg gggattagtt |
| 77881 | atggtctaat tggccacagc cagagcatgg ctatgttaca gtgtgtggct cattttcaaa |
| 77941 | taactttata tgcagtatag gtctggtata taatcccaaa ggaaagagcc caaaacactc |
| 78001 | aaatgaagtc aaaagactta tctttactta tgggttgcaa cactaaaaga ttttctatgc |
| 78061 | actactcttc gtcttccttg tttcttttct tttttttttt tttttttttt ttgagacaga |
| 78121 | gcctcattct gttgcccagg ctggagtgca gcggtacaat cacagtttac cacagcctca |
| 78181 | acctcccagg ctcaagcgat cctccttcct cagactcctg agtagctggg accacaggca |
| 78241 | ggtaccacca cacctggcta atttttgtat tttttgtaga gacagggcct tgtcatgttg |
| 78301 | cctaagctca tctcaaactc ctgggctcaa gtaatcctgc ctctgcctcc caaagtgctg |
| 78361 | ggattatagg catgagccac catgcctggc cccttattta atttcatctg gttcctactg |
| 78421 | tccttcaatg attttgacaa tgtagttcct acccacacac cacaataata caaaaccaaa |
| 78481 | gcaaactcca ctatgtctgc tgctaaacac ccccaacaaa cacacataca catcacacca |
| 78541 | caccacacaa cacaacacag gtgtctcctt cccttttgct agcagattcg ctaggaagca |
| 78601 | gctgcacatt tccagcaagt cgaacaaact gctgagagct agggcccctg accattgtgg |
| 78661 | gaaaggaagg ccaattctcc cctctagatt gtacccttct gaagatcctg gccatgaaag |
| 78721 | aagtcatttc tccagagaat tttaagccat tcagttccta tacggcaggc aggaaaggtg |
| 78781 | tcatcctccc catatcatca gtggaggaac tcagtgtaac ttgtttcatt tcctcttact |
| 78841 | tcaaaaSgtc attttaagta atagggtcat ttaggtaata tcagtgatct ttttaatttc |
| 78901 | tcctcgcatt ctctcccMaa ttttggtttt acaaaataat ggttaaaaaa aaaaagctat |
| 78961 | catactctcc acgaaattta acttaatcta gaattttgaa tgagaaatta ataatcaaag |
| 79021 | aacagtgcat tttaggccag gcgcggtggc tcactcctgt aatcccagca ctttgggagg |
| 79081 | ccaaggcgag cagatcacct gaggtcagga gtttgaRacc agcctggcca acatggtgaa |
| 79141 | accccatctc tacttaaaat acaaaaaagt agctgggtgt ggtggtacgc acctgtaatc |
| 79201 | ccagctactt gggaggctga ggcaggagaa tcgcttgaac ctgggaggtg gaggttgcag |
| 79261 | tgagccgaga tcgtgccatt gcactccaga ctgggggaca agagtgagac ttcgtctcaa |
| 79321 | aaaacaaaca aagaacagtg cattttctta cacaaataag caggtattaa gtggttctct |
| 79381 | ggataggaat ttggcaatac aaatcaaaat taatgcctat aatcctagca ctttgggagg |
| 79441 | ccaaggtggg cggatcacct gaggtcaaga gtttgaaacc agcctgacca acatggagaa |
| 79501 | acccgtctta aaaaaataca aaattagctg ggcgtggtgg cacatgcctg tcatcctagc |
| 79561 | tactcgggag gctgaggcag gagaatcact tgaacctgga acaggtggag gttgcagtga |
| 79621 | gctgagattg tgccattgca ctccagcctg ggcaacaaga gcgaaactcc atctcaaaaa |
| 79681 | caaaacaaaa ccttgatatt tgggccaggt gtggtggtgg cacatgccca aaatcccagc |
| 79741 | actttgggag gctgaggtga aaggattgct tgagcccagg agttcaagac cagcctgggc |
| 79801 | aacatagtga Sacctcatct ctacaaaaaa taaaaaatta gcYaggcatg gtggtgcaca |
| 79861 | cctgtggtcc cagttactcg ggaggctgat gtgagaggat cacttgaacc ctgaaaggtt |
| 79921 | gaggctgtgg tgagctgtga tcataccact gcactctagc ctgggtgaca gagtgagacc |
| 79981 | ctgtctcaaa acaaagaaac aaagaaacaa aaatgttcat atttggctgg gtatggtggc |
| 80041 | tcacacctgc actttgagag gccgaggcag gatgatctct tgaagtcagg agttcaagac |
| 80101 | cagcaatctt ctcgcctcag cctcccaaag tgctggtgct aggatgatgg gtgtgagcca |
| 80161 | ctgcacccag cctaagatag taaattttgt tacatacatc ttaccaaaac tcaaaaaaaa |
| 80221 | aaaaaaaaaa aaaaagctgg ctgggtacaa tggttgaagc ctgtcatccc agcactttgg |
| 80281 | gaggccaagg taggaggatt gcttgagccc aggagttcaa gaccagcctg ggcaacacag |
| 80341 | tgagaccatg tctctacaaa aaatttaaaa attagccagg tgtggtggca tacacctata |
| 80401 | gtcccagcta ctcaggaggc taaggtggga ggatcacttg agtctgaKcc tgggaggctg |
| 80461 | aagccataat aagctgtgat cataccactg cactctagcc tgggcaacag agtgagatct |
| 80521 | tgtgtcaaaa aaaaaaaaaa aagaaacaaa aagaattaaa aaaaaaaaaa tctcatgttc |
| 80581 | tttcatcaaa taattccaat cacagaaata aaagtttagg aaataatctg aaatacaaag |
| 80641 | atatatggac attaatagaa ttatttatca ttctgaaaaa ctgaaggaaa actaaatatc |
| 80701 | taacaataac ttaaattatg gcaagaatat acatatgatg aaatcttatg cagtcactta |
| 80761 | aaatgtttta aataagtttg gctgggtaca gtggctcacg tctataattc cagcactttg |
| 80821 | gaaggctgag gtgggatgat tgcttaagcc caggagttca agaccagcct gggcaacata |
| 80881 | gtagactcca cttctacaaa aaataaaaat aaaaaaaata gctgggcatg atggcatgcg |
| 80941 | cctgtagtYc agctacttgg gaagctaaag cgggaagatc acttgagccc aggaggtcaa |
| 81001 | ggctgcagtg agctaggatt gcaactgtac cactgcactc cagcctgagt gacagagcaa |
| 81061 | ggccccgtct ctggggaaaa aaaaaaaagt tttaaataat tttaatgaca tggggaaata |
| 81121 | ctcaagaaaK gttgaatttt aaaagcagga tataggctgg gtgcagtggc atatgcctgt |
| 81181 | aatcccagcc ctttgggagg ccaaggcagg tgaatcgctt gggctcagga gttcaagacc |
| 81241 | agcctgggca acatggtgaa accctgcctc tacaaaaaat acaaaaatta gccgggcatg |
| 81301 | gtggcatgca cctgtagtcc tagctatttg ggaggctgag gtaggaggat tgcttgagcc |
| 81361 | cagaaggttg aggctgcagt gagctgtgat cgtaccactg cactgaagcc tgggtgagag |
| 81421 | agtgagaccc tgtctcaaaa caaacaaaca aNaaaaaggc aggataaaac tattatggta |
| 81481 | tggccccatt tttgaaacta tatatacaaa tatttataga cagatagata tatcatatac |
| 81541 | atatttatcc ctctccccac atacttgtga gtttgtatat gtgtatgtat acgtgtaagg |
| 81601 | aagtatacca aataatgttc atttctgagt tgtggaatta tgggtgcctt ttactttccc |
| 81661 | ccttaacaca ttatttttca tgttttctaa atagtaccat catatacaat taggaaaaaa |
| 81721 | ttactttcat attgtttaat ctagtaatct atttgtagat atgtatcata agaaaataac |
| 81781 | caaaaacgta gacaaaaact tatgcataat aatgttcata taaagataca cagaaaacta |
| 81841 | ttaataaata atggttactt ctgcagggca gaattagggt aaagacttat ttggggactt |
| 81901 | atttgttata tctattacaa aaatgatttg aaaaaaattc cattattatt tgaaaaaacc |
| 81961 | tgaaagcata taaaaatcct atatgataga ctattatgca gccactacaa atatttatga |
| 82021 | agagtatata taaatttata taatgaaaaa atggtaaatg gggaaaaaag cagaatataa |
| 82081 | aattttaggt aatatatgat ctggaaattg caaaaccaga aatatacaag cacaaaatac |
| 82141 | tgaaagaaaa taggctaaaa taagaacagt agttttctcc agatagtggg attacgggtg |
| 82201 | atttcagttt tcttccttat acttctctgc gtttttcaca tatcctagaa tgaatatata |
| 82261 | ctatttttca atcaggctta aaaaatgttt ttggtttctg ccctataatg aaaagcctgc |
| 82321 | tccttgtgtg aaattttatc cacaaaggat tatcagtagt aagcaaccca ctagcaagaa |
| 82381 | gactgttcat aatgtatatg cctaagaaaa tcatctttgg tgggtcagtg agctcagttt |
| 82441 | aaagggacac agctctgacc acttcctgcc ttcttgcttt tggagttgtt ttagtaaata |
| 82501 | taactgagga atcaagttct caaacatgag atgcctgaca aacttaatac tgtactgtaa |
| 82561 | gaaccaggac caccaccttc tctcaaccta acctYtaaga ctttacactt aagaatagac |
| 82621 | aagaccgggc cgggcgtggt ggctcatgcc tgtaatccca gcactttggg aggccgaggt |
| 82681 | gggcggatta cctgaggtca ggagtttgag accagcctga ccaacatgga gaaaccccat |
| 82741 | ctctactaaa aatacaaaaa ttagccgggc gtggtggcac atgcctgtat tcccagctac |
| 82801 | tcgggaggct gaggcaggag aatcgcttga acccaggagg cgggggttgc ggtgagccga |
| 82861 | gatcacgcct atagcctggg caacaagagc gaaactctgc tcaagaaaaa aaaaaaaaaa |
| 82921 | gaatagacag gacctaaata acatagattg ggtcatgtta gtctgtaaat caaaaacact |
| 82981 | taacagctaa ttacatgaag aataaaatcc aaatttctta gcataacgct taaggttctc |
| 83041 | taagatccaa ccctcaaatt tccttcccag cctcatcttc agttcattcc ctaaaatatc |
| 83101 | ctctacctca ggcacatttt ccccatacat gccaggcatt ttccaagctg tgtgcctttg |
| 83161 | cccatggtgt gacctccatt aaatgccctg tctcctaaac ctactgaact cctattcttc |
| 83221 | ctttacaacc tggctcaaat gccttctccc aaactctcct tatcgccccc accatcagga |
| 83281 | acaattgttc tttcctctat gtgctcccat attgcttgtt cctctacaaa cttaattaca |
| 83341 | atcctccctg ggtggatgct agctgtaata cttgtttcat tttactaaac tcctggaggg |
| 83401 | cagaaaccat tttaaaatca tttccactcc agcacactgc ttgcacacag taggtactaa |
| 83461 | atatgctgcc tgagtaaatg acagccaatt ggcccttatc tgaattcttt cagtggtaca |
| 83521 | gccagggatg gcaaattgtc aaagctagtc cctctaattg caagttatac atatagcatg |
| 83581 | agtgaccagc agaaaatgcc aagggcctgc cactttaaga ggactggcaa gaYgactatg |
| 83641 | tttacacaat cctaattaaa actaaagggc aatgagggaa agaagagaaa tagggagatt |
| 83701 | aagaggtgtg gattctacag gtcacctcct cttcttagtg agcttgctag gcactaggct |
| 83761 | ggaagatctg agctacaaag tagcctattt tatctttggg gtagcctgta ggatctaagt |
| 83821 | attgaaaagc aactaattag gccactttgt cctccagcaa ctggccctgc tcttgggcat |
| 83881 | taacttccaa ctggtacaca aggctttttc tctaaatgaa tcccactgta ccaatcacta |
| 83941 | ggagaatccc tttaaaaaag gcttgaaatt aataacagca ggaaacaaag tctctcaatc |
| 84001 | ctctgaatca caatatccac actgtccatt ttgatgaaat tcacccccac ctccttcaaa |
| 84061 | caataggtcc taccaaggaa tgacttctct caggaccaac atgtcctgtg attgtttttc |
| 84121 | tggagcaagc ttccagtgcc tgcttctcat taaatagcta cttaatgtct gatcacccag |
| 84181 | ctgtgactat aagtagagag taaagtaact ataatttagg aRgtaaaaaa gaaattccac |
| 84241 | tcctacccca agtccagtag tcaaacagct tagaatagac atttccctac ctcaaacctt |
| 84301 | cttaggctta tccaattttc agatctagaa tttttagcat ctcttaactc tttcaatata |
| 84361 | aaaatgagga aaacaatRtY tagagtaaga aagagaattg caaatgatta tacagtaact |
| 84421 | tagtcacaga atcttttgac caggctgtct aacacctcat ttaactgatg agttttcaag |
| 84481 | accactgttt ccaggaacag gttacagggc actacaatat aatcagctta atttgcatat |
| 84541 | attcaagata acagccccta ggcttgaccc cttcaccatc ttcctgccta ccttttttca |
| 84601 | aacccacaac tgctaacaac aacaaaaaaa tcaatgtagt gatttaatac atcatttctg |
| 84661 | cctaaaaaaa gactggagct accaaaccat ctgcttcagc ctcaccacct gggattcatc |
| 84721 | ttgattgatt tgggtgtgtt ggtgggcagt ggctgccaca gcctgaagaa ctcttccaac |
| 84781 | tctttctggg ggtcttctgg agatgggact gcaatggctc ctcagctgca gtttcctata |
| 84841 | gagcaacatg ttgaaacaca cttgttccct caacacactt gtgtttcagc cctcaacaca |
| 84901 | cttgtgattt gggaggagta ccaggagaaa gaccagtttt tggcagggcg cggtggctca |
| 84961 | cgcctgtaat cctagcactt tgggcggcca aggcgggtgg atcgcttgag gtcaggagtt |
| 85021 | caagaccagc ctgaccaaca tggtgaaacc ctgtctctac taaaaataca aaaaattagc |
| 85081 | tgggcatggt ggcacatgcc tgtaatcata gctactaggg aggctgaggc aggagaatcg |
| 85141 | cttgaacccg agaggtggag gttgcagtga gctgagatcc cgccattgca ctccagtctg |
| 85201 | ggtgacagag agagaccctg tctggRaaaa aaaaagaaaa gaaaggccag ttttctccat |
| 85261 | tatctagtca ccccaaccta taagcaagca tatgcacagt cactataaag gaaagtgact |
| 85321 | tgttttccag cagacatttc taatcttatt aaatcaaatg aaaactgcaa tcaaaatact |
| 85381 | ctccttagga agttatttac ttattctaac aaaacacttc tttgggagct gcctttaaag |
| 85441 | tctactccat gtgagacaaa cagaaaaatt cggtgttact gttttatagt cacacctcat |
| 85501 | ccttcaccaa aaaaggtatt gccttgcatg aatctttggc tcacggcatc ttctggctgt |
| 85561 | ttctaaaaat tgaatccacc aagctaagag taaagatttg ctacctctga ggatattcaa |
| 85621 | aagattggct cagaggcagt ctgaggcaat gacaccatca ccaaaggaaa tctaaaggct |
| 85681 | gagaacacaa ttactttgaa ggggtcaaac ttaaaaacat tctaattgcc ccataattac |
| 85741 | aatcaagggg gcaagacatg tacaatatct gtcacatttg ttcaactatt atatcacttt |
| 85801 | ataattacac attaYataat gataaatttt ggccctggaa gaaatcttca agatcatata |
| 85861 | atcctaactg tcccttttta catatgagaa aagttcagtc cattgaggtt aggcaatttg |
| 85921 | tccctggtca cacatctggt tagcaacagt caggatgaga aacccagaca cccaaactcc |
| 85981 | cagcataatg cacccacaca tacttatgta tacagctcct cctcccctgc ttctcacttc |
| 86041 | cttcactccc ctgatcaaaa gggacagaaa taacttggga cactaaagca caccaagaat |
| 86101 | ggttctgaaa cccagtagca gagaaatgat ggcttaaaaa ccaggacttc tctgcagtgg |
| 86161 | cctcaatggc caatgtttag tgtctggctc acaagagttc tgggcaatct acctcagagc |
| 86221 | aagggccaat gaaaacctct ggacaccacc ctcccacagt tagaaaaata tctttattaa |
| 86281 | acctctctgc tctcctccta cagtaacctg tttgcctcaa ataattgaaa gacaaagaca |
| 86341 | atcactttga agaatctggg tcagggaaaa agtaaatggt agagatcctg ctcaggacaa |
| 86401 | agctgaccta tSaaaaacta gagggcagaa accattttga cttacctcct ggtggaagcc |
| 86461 | tccacacact agaacagggt gatctccagc agtctggctt agaattagtt tccacctagg |
| 86521 | gaaggggcaa agggacagga atgattattc attcccctaa cccacccact cttagtgagc |
| 86581 | tcagcctcac attatcctgg gcaaccccag cgttctgtgg atcagacagc aagcagaagg |
| 86641 | gaagccatgc aggccaggtc aacggggtga gtcagtggca gagcaggaag agtcactggg |
| 86701 | gcagaaattc tggccttctg tggcaagact gccaagccac acccacactt ggtcagcact |
| 86761 | gtgaaggccc tgccctgtta ggatttcagt ctttaagcca gaaaRcttct tcctcaggag |
| 86821 | gccccacacc actctcattt tcaagtgtct ctcctcctgc tcaaaaatct tccagttttc |
| 86881 | ttctccctaa agcatcatat ctaaattctt tggcttaact ttccaaatcc tccattatat |
| 86941 | ggcctcaagt catttattca accttatttc ccactattct acagaatata ctctgaattg |
| 87001 | atcaagccag tcttcccact tctctctgcg tgagccaagc cttttctcat ttccctgctt |
| 87061 | tcaattatcc aggagtggta aattcaaaca tcttcagggg tcaggcaggt aacagaaaca |
| 87121 | aattatccag gcaaggccca gtggctcaca gcggtaatcc cagcactttg ggaagccaag |
| 87181 | gtgggcagat cgcttgagct caggagttcc agaccaacct gggcaacata gtaaaactcc |
| 87241 | gtctctacaa aaatacaaaa aattagcYag gtgtggtgga gtgtgcctgg gtcccaccca |
| 87301 | cttgggaggc tgcagtgagc tgagaccgtg ccactggact ccagcctggg caacagagcg |
| 87361 | aaaccacgta tccaaacaaa acaaaatgaa tgatccagtg gggcacatgg cagatctagg |
| 87421 | aaacgctgca cccccctaaa ggcattcaaa tattttaatt caaatattta tgtagtgtct |
| 87481 | cactgtgagc atcacgataa ccttttaaat agttattaat actctatctt gcaaatgaga |
| 87541 | aaacttcccc aaggttaggt aactggcaaa atgctgggca cagtctctag acctgtctga |
| 87601 | tgccaaagca agagctctac accattaggc ttctagcagt gcaaacacca tggaagatac |
| 87661 | agaaccctca aagtcacttt tcatgataca caaccttaat agtaggatgg gaagcccaaa |
| 87721 | tcacagctta tctaaccata tcccaactgc ctccaattct gttcattttc caaaccccat |
| 87781 | ctttctgcca aagtatttca ttcctagtta tccaagtcct actgaattcc ttcttctgta |
| 87841 | aattcctata gggtttatag attagacaac acaaataagt gctaaattat atactgtctt |
| 87901 | gcaaaatcag atactgcttc tatgtatttt agtgtcagcc tcatcaatca aactattgta |
| 87961 | ttaatctata tagttcttct accaaaaatg cctaacctga atctaatcaa gaagaatcag |
| 88021 | acaaatgcta tgaaagacaa aaaggcattc agattaaaaa agaactgttc aaagttaaag |
| 88081 | cagactaaag acatgatagt catgacaact aagtgcataa atacatgcat gatccttcaa |
| 88141 | tgaatcctga attttaaaaa actcaataaa agacatcact gggaaaagat gagaaatctg |
| 88201 | aacaagtatt atatactgga tgtattgaat caatgttaag tttcctgagt gttaatagta |
| 88261 | ctgtagttat ataggaagat acctttttct taggaaatac ttgctgaaat agggattaaa |
| 88321 | taacatgata tttacatcta acagtcaaaa agattcagaa aatgaaggaY aaatagacag |
| 88381 | caaatttggc aaaatgttaa tagtgactct aaatgagaag tatataagta tttgttacat |
| 88441 | tattttgcaa aatatctata ggtttgaaaa ttttcaaaat aaaataattt agtaaggagg |
| 88501 | gaaatcctgt attatatact tttttatata ctcagctctg tgacagatat aataggcact |
| 88561 | cattacttta ggtatctgct cactacctaa ggaaactggg catttggagg tcaRcaatca |
| 88621 | gacagagtcc actttgctgc agctcatgag gggtatagta ggaaattctt agggaataag |
| 88681 | aggtttctcc aaaactcaca ttgtcacacc acaggaaatc tagaaaagca ggcaattacc |
| 88741 | tcactcctca ttgaggccat cacagaagta caggcactga acaaatgccc actcgctagg |
| 88801 | cagaatgcag ctgctgccca cacgttagta cccagtggta aggtggatac aatgtgacct |
| 88861 | cagaggacca agagttgagg tcacattgca tccaccttgc caccgggtac caaaaatttg |
| 88921 | acaatctgca aaacaagtgt ctctggccag tcacaggaag gcactcaagt atcaaataaa |
| 88981 | catgcctgaa atatatgcag tctttcctcc tcaccagata aaccagtcct cagctgcaat |
| 89041 | aaggcagaag aaatctgcaa ggctgaatca ctggtatttc ctgtgcagac ttcaaagaaa |
| 89101 | ctggcccttc aataggacag aaagggcagg cgacacagct gccaacagag gcctcaaccc |
| 89161 | aggagcatgc tatgcaataa tgccttctct cccttctaca gccatcagat acctcatttt |
| 89221 | taYttctgcc aaatggcccc ttattaccgc gactgattac ctaatagcca accctttacc |
| 89281 | ttggttctct cctcccctca atgagaccca agatactggc ctttctagac agctcctcct |
| 89341 | ccaatttgcc agaggaattg tcaaaactat tcccctggcc aggcRcggtg gctcacgcct |
| 89401 | gtaatcccag cactttggga ggccaaggcg ggcggatcac actgtcagga gatagagacc |
| 89461 | acggtgaaac cccgtctcta ctaaagatac aaaaaattaS cYgggcgcgg tggcgggtgc |
| 89521 | ctgtagtccc agctactcgg gaggggaggc tgaggcagga gaatggcgtg aacctgggag |
| 89581 | gcggagcttg cagtgagccg agatcgcgcc actgcactca agcctgggtg acacagagcg |
| 89641 | agactccgtc tcaaaaaaaa aaaaaaaaaa aagctgccta ggccaggcac aatggcttac |
| 89701 | acccataaac ctagcacttt gcgaggccga ggcaggagga ttgcttgagc tcaggtgttc |
| 89761 | aagactagcc tgagcaacac agcaagacct tgtctctact aaaaatcaaa aaaattagcc |
| 89821 | gggtgtagtc ccagctacac tacttaggag gctgaggtgg gaagacagcc actgcactcc |
| 89881 | agcctgggtg acagagcgag accctgtctc aaaaaaaaaa aaaaaaaaSt tgcccacagt |
| 89941 | aggaggtaga ggagaggcca ttattaagca cactctcagt aaaccatgaa tctgacagag |
| 90001 | ctacccaaaa catcaatgca gtcttaagct gcattagtag aagatgactc tatgccccac |
| 90061 | ctttgaagag caaagttaga ggacttgcag aggaagaaca ccaatgacca tggcaaaggg |
| 90121 | tctagaagta tatcatttca aatatggagg aagaactaca cacttttagc caggggagaa |
| 90181 | caagaaaact tttttcaaat atacgcatag ctttcacata gatgaggaga aagacttatg |
| 90241 | agtccactat aagaatacaa aaccagaact aatgagtgga agttacagga agactgattt |
| 90301 | tggtttgcca cagggaagaa atttcagcat gttgtagaag ttcagaatgt gggatgtaga |
| 90361 | acccagaatg cttggattag gatgtcaact ccaccactta taaactgtgt ggctttgggg |
| 90421 | agattaatct acctgtgcct cagttgcctt ccctgtaaaa gagaatagta ataatagaac |
| 90481 | ttagctcaca gagctttaag aagtagcttc cgagRctggc tgcggtggat caggcctgta |
| 90541 | atcccagcac tttgggaggc tgaggcaggt ggatcacctg aggtcaggag ttcaagacca |
| 90601 | gactggccaa catggggaaa cccgtctcta ctaaaaatac aaaaaaaatt agctaggcKt |
| 90661 | ggtggcgcat gcctgtaatc ccagctactc aggaggataa ggcaggagaa tcgcttgaac |
| 90721 | ccaggaggca gaggttgcag tgagccaaga tcacgccatt gcactccagc ctgggcacca |
| 90781 | ggagcgaaac tccatctcaa aaaaaattaa ccagacggta gtggtgtgcg cctgtaatcc |
| 90841 | cagttactcg ggaggctgag gcaggagaat catttgaacc tgggaggtgg aagttgcagt |
| 90901 | gagcagaaat cacMccactg tactccagtc tgggtgatag agagaggctc catctcaaaa |
| 90961 | aaaataaaaa gaagtagttt ctgtaaagta cttagaacag tgcctagttc acaatatacg |
| 91021 | ctggaaaaaa aatgttacct attgctatta ctaaatgttg atacaacaga ccaggctggg |
| 91081 | tgtggtggtt tgtgcttgta atcccagcac tttgagagaa ctgcttgagc tcaagagttt |
| 91141 | gagaccagcc tgggcaacat agtgagacct cgtctctact aaaaataaaa agagttgcca |
| 91201 | ggcacagtgg cgtgcacttg tagttccagc tactcagcag gctgaggtgg gaggatagct |
| 91261 | tgagtccagg agatcgaggc tgcagtgagc tatgatgacg ccactgcact ccagccagag |
| 91321 | tgacagagca agactctgtc tcaaaataac aaacaggctg ggtgcaatgg ctcacgcctg |
| 91381 | taatcccagc actttgggag gctgaggcgg gtggatcacc tgaggtcagg agttcgagac |
| 91441 | cagcctgggc aacatggtca aaccctgtct ctactaaaaa tacaaaaatt agctggttgc |
| 91501 | agtggcacgt gcctgtaatc ccagctactt gggaggctga gacaggagaa tcacttgagc |
| 91561 | ccgagaggcg gaggttgcag tgagccaaga ctgYgcaatt gcMctccagc ctgggcgaca |
| 91621 | gagtgagact ccatctcaaa ataaataaat aaataaataa ataaataaaa taaaaataat |
| 91681 | atacaataca aaaaacagac cagataacct gaagcccttc ctactaaata tgcctagaag |
| 91741 | tgctcagaaa aatataataa acatccttYt agactgtgcc agctattaag gtattgtctc |
| 91801 | tcagctccaa atctaccctg ttatgctccg ctttgtgatg ctggggcctg gactccacta |
| 91861 | aaccccattt cacatttgcc agctggatcc ctgtcaacag ggggtgctaa ggggaaaaca |
| 91921 | gaagacaaga gaaggctaca agggacttcc tccttcttgt agcctggtgt ttcttgacaa |
| 91981 | cagaagttgg ttcttcccag ttgcYttcta aactcccaaa accagcctcc tgcccctcaa |
| 92041 | aggcaccagc atcagctaag taaggctcaa cagaagccag ctcctccagg ttcctcctat |
| 92101 | aagggcctga gaaaccagca gcaccaggta acaccctctc cagagaatct gggtacagct |
| 92161 | attccagatc cctctagact tctaggttct ggtagcccca accttcctcc ctttgttttt |
| 92221 | cagtcctgga tgagaaaaat tctttatgca gttattatat tgaagttact ttaatattcc |
| 92281 | ctttctgctt tttcagccct ccaccaaggg tttaaatcaa tctcctttat ggagttcact |
| 92341 | ctattgtgtg gtttctgttt tgctgattgg atctgaactg acatatagat aaactgagct |
| 92401 | ctcaagaaag taagaaaaat ccccaggaac cagaaacaaa ttagtgagga ggattagcga |
| 92461 | gctgatgaca tagctgtcct agccgtgtat ggaatttgta acaccctcat aggttcagaa |
| 92521 | gacagatctt gagtccacaa aaggcactga gatccctgtg cagaggctga gattcttaag |
| 92581 | tttatactca gtaagataat agactaggaa aaaaaatctg tccagagaga aataaaaaac |
| 92641 | ttgccttgct cacctagtga gaaaggaggg aaaaaccgtc taggaattca aaacaggctg |
| 92701 | ggtgaagtgg ctcacgccag taatcccagc acattgggag gctgaggtgg gtggattgct |
| 92761 | tgagcccagg agttcgagac cagcccgggc aacatggcaa aaccctgtct ctacagaaaa |
| 92821 | tataaaaatt tgccgggtat ggtggtgtgc acctgtagtc ccagctactt gggaggctga |
| 92881 | ggtgggcgga ctgcttgagc ccaggaggca gaggttgcag tgagccaaga tcctgcaact |
| 92941 | gcactccaac ctgggcaaca aagccagaca gatcctatct caaaaaataa aataaaataa |
| 93001 | aataaaattc taggaagcag acattaatat gtagtgaaaa acaaacaaac gatcagtggt |
| 93061 | tcctggtatc agggtgaaag gaaaaacgga ctgtaaagaa gtatgagagc cgggcacggt |
| 93121 | ggctcacgcc tgtaatccca gcactttggg aggccgagga gggcagatta cctgaggttg |
| 93181 | gcagttcaag atcagcctga ccaacatggt gaaaccccat ttctattaaa aatacaaaat |
| 93241 | tagctgggca tggtggtgca tgcctgtaat cccagctact caggaggctg aggcaggaga |
| 93301 | atcgcttgaa cccaggaggc agaggttgca gggaSccgag attgcaccac tggactccag |
| 93361 | cctggagaat aagagtaaaa tccatcttaa aaaaaaaaaa gcatgagaaa tcttttgggg |
| 93421 | gtgagagaaa tcctctgtat ctKgattatg gctgctgttt acaggtgtag acatctgcca |
| 93481 | aaactcaact ttcttgaaaa ttattacaga cacaaaattg aaaacatgag acaaaaacaa |
| 93541 | gatcacgaaa aacaagcaga tttgaaaatg aaccaaagag cacttctaat aaatgaaaaa |
| 93601 | gttagctaat aaaaataaga attcaaaaaa caaattagac ctagctgaag agtaaactag |
| 93661 | tatactagaa gatagatata aggaaattat ccagaagtta acacagaaag atagaaatgg |
| 93721 | aaattatgaa agaaagaata agatgtagac Racagaatga ggaagttcaa cataMgttac |
| 93781 | gcatgagatc tgagagaggt aaagaggtaa tgattgggaa tttttacaca attagtgaaa |
| 93841 | agatatgaaa tttcagatta aggaattact atatgccccc agtatatagt gatttaagaa |
| 93901 | aattaaattc acaccatgta atggaagtac agaacacaaa tgacagcaaa gaagatctta |
| 93961 | aaaacaaaca gaaagaaaga caggttaccc acaataaaat aagttaattt gcaagcatag |
| 94021 | ttctcaagag taacaacaaa aagccagaat ttcaatgaaa aaacactttc aaagtgctaa |
| 94081 | gagacaataa ttggaagcct agtacccagc taaactacca atcaagactg aaagtgagct |
| 94141 | gggcacagtg gctcatgcct ataatcccag cactctgaga ggccgaggcg ggaggatcac |
| 94201 | aaggtcaggg gatcgagacc atcatggcta acacaatgaa accctgtctc tactaaaaat |
| 94261 | acaaaaaatt agccaggcgt ggttgcgggc gcctgtagtc ccagctactc gggaggctga |
| 94321 | ggcaggagaa tggtgtgaac ctgggaggcg Kagcttgcag tgagccgaga ctgtgccact |
| 94381 | gcactccagc ctgggcaaca gagtgagact ccatctcaaa aaaaaaaaaa aaacaaaaaa |
| 94441 | ctgaaagtga aataaagatt ttttaaatga agacagtgtt taccactaac agattcccac |
| 94501 | tggacaaaca tctgaaggat gtatgtatct atgcaataaa aaaggaagga ataaaatgca |
| 94561 | aggaaaaata gtgaaaaagc aaacagtaga catacatggg taaaatgaaa taatcactaa |
| 94621 | caatacaaaa caataaacat attttaattg tgttggggtc tcactctgtt gcccaggcta |
| 94681 | gctaagaact cctgggctca agccattctc ccgcctgctc ctcctgcctc agcctccaga |
| 94741 | gtagttggga tgacagggca agccaccatg cccagctaca aaataataat tatcaattag |
| 94801 | aattgttcaR cttagagatg gattgacttt gggaatgatc tcattctaga aagtaYtcat |
| 94861 | tttgagtctg gacaacacct tttggtatgt ttagtgtaca ctgggtaaga ggctaaatta |
| 94921 | aatgaccaca ccatatatta ggttaatgca aaagtaactg tggtttttgc cattactttc |
| 94981 | aatggcaata actgcaatta cttttgcaca aacctaatat cttctgatca tagttttatc |
| 95041 | aattagacac agctgtccaa acagcccctg atcagctctg ttcagccacc tgagcagggc |
| 95101 | tttgccctca tcctaaaagt tctagaattc aagtcttatt tcaaggcatt cttcccactt |
| 95161 | ccatgttcca catgtaaaaa agaattctac gtttcttaac gagctttcag gtaagatctt |
| 95221 | taagcttttg gaatgtaccg cttttcgctg tgaactaaga gcttagacat tacaattagt |
| 95281 | catcccttgc tagaacttag gctacgaaaa gaatactttg gaagctttca caaaaaagta |
| 95341 | tgtggttctt cctctaaaga agaaaatacc acacagaaag agggactgag gaaacataaa |
| 95401 | cccatctgtg ctgacagaaa gctaccttac atgatcctca tgttcgcccc aggttcccat |
| 95461 | atattcacag cagtaaagat ctccacaaag cagagaggcc caactccctt accattaaag |
| 95521 | ctttaacgac cagacaaccc agaagtttgg aaacatagaa ccaggcaaca gatactagag |
| 95581 | agagggaatt atgtattaaa gcaagaaaat atcatcacgt tttcaagagg gagtgccatt |
| 95641 | ccagtaaaga ggacagctta atactgaggt tcaaatgcca ctaaatgaaa acgggaaagc |
| 95701 | gaaattcctg tgccctttca gcccaagaaa ttattccaaa gcatacattt attcccatta |
| 95761 | catgtcgcaa atcctgccta gccattagga tacagtgacg aaaaagtctc tcgttatcta |
| 95821 | gaagggcaga caaacacgca gacttttaaa ataccagatg gcaggccggg cgtgatggct |
| 95881 | cacgcctgta atNccaagca ctttgggaag ccgaggcggg cggatcacga ggtcaagagt |
| 95941 | tcaagaccgg cctggccaag atggtgaaac cctgtgtcta ctaaaaatac aaaagttagc |
| 96001 | cgggtgtggt agcgggcgcc tgtaatccca gctactcggg aggctgaggc agggaactgt |
| 96061 | ttgaacccgg gtggcagagg ttgcagtgag ccgagatcgc gccactgcac tccggcctgg |
| 96121 | gcgatagggc gagactccgt ctcaaaaaaa aaaaaaaatg acaagtgcta actcagaggt |
| 96181 | aagcaaaaga gagtacccag aggcggcagc atcaacccag tgggctcccg cccacccaca |
| 96241 | atcttcacac agtcacacca cttatagtca taaatttgta ctctccgagg tgcgtgtatg |
| 96301 | aaacatgtta tctgtgagag tccatggatg aaaaacaaaa acaaacaaaa attaaaaaaa |
| 96361 | aaaaaaaaaa agacttaacc tgttttgacc aggacaacca ccccaggaag taggtaaggc |
| 96421 | aggtaaggtc attgtttaca tatatgaaaa gtgaggctca aaatacactg accagtccaa |
| 96481 | gacaacgcgc gcccccacaM agcaccttcc ccgtccccat ttatgcccaa cccataaacc |
| 96541 | caaagtagag ccaacctcgc actggctacg taacccgggc caagctaaac attataaacc |
| 96601 | tcaggctccc catctttaaa gtataacagg tggttagaag attaaaaaga taaacatttg |
| 96661 | gcaggtcatt gtggacggtg tagctcagaa aatgctgact actgcactac cataactact |
| 96721 | gctattgtta acgataaaag gcaaagactt tccaggaccc gacccctcgc ttacaagcct |
| 96781 | ttcgggaact tgccctatac ccccgacccc agtcaccttc agatttaatt ccacagacac |
| 96841 | gcccccaaac agaccctccc ctttaaggca cccccccccc ccccggctcc tccctctcag |
| 96901 | gcgcctctcc tcacaaacct tacccccata gattctgccc tttcggactc ggttatggag |
| 96961 | gaggccttcc gctcgcagcg gcctctccag cacccctttt aggcctgagc acccgcaggg |
| 97021 | gtgcccaggc cgcacgccaa acgcgggccc gcgccggtta cctggctgcg cgctcccgct |
| 97081 | ctgcctcgcg attctccgga atcgtacctc ttcgtgggct cgcgccagcg ctgtgagcgc |
| 97141 | acaattagtt taaactatgg ccccgccccc tcgcgctgcc ctctgattgg cctctgtcgg |
| 97201 | cggggcccgc ttaaggaccc gggaaaagga agtttgaggg ccactgggaa agagaagggg |
| 97261 | tatcaccgct tccggacccc ggcctgtact tgaaggcaaa gagaactaca aatcccagcg |
| 97321 | tcacccgcgg ccttgaagcc ccgcccctga caaactgaag gtcccggtaa gcatcgcgtc |
| 97381 | agtacttatg gcgcctgccg ggttgtggtg acgaaagcag ttgccatgga gttgctctga |
| 97441 | gtaaccctga ggcagtggga cgccaagact ggagaggaag cgactgcggg tgagtcgggc |
| 97501 | gggaaaacag gaaacccgtt ctaggggaca agagatctat ggagaaggga ggggcactca |
| 97561 | cagaacgact aagaaccttc ctgctcSggg atctgagatt tcccccaccg accctcatca |
| 97621 | cctttcacRg cgaacaagat acaacactca catgggacag actgggcgag ggggagcaga |
| 97681 | gggagggata ggcgttgaga tataYatcca tacagatttg gggatgctta agaaccaccc |
| 97741 | ccagggccgg gagcggtggc tcacgcctgt aatcccaaca ctgggagacc aaggcgggcg |
| 97801 | gaccacttaa ggccaggagt tcgagaccag cctgagaaac atggcgaacc ctgtctctac |
| 97861 | caaaaacata aaaaaattag ccgggcttgg tggcgcgctc cagtggcccc agctactagg |
| 97921 | gaggctgagg tagaaggatc gcttgagccc ggagattgaa gctgcaRtga gccgtgatca |
| 97981 | cgcaactaca ctccagcctg ggctatagag cgatacctcc atctcaaaac aaaacaaaac |
| 98041 | gaaacaaaac aaaaacaaaa caaaacaacg aaccccgccc ccaacgcaaa cataaagctc |
| 98101 | ccaaaatcgg atgcttcaag ttccaRgcat aacctgggag atcagaaacg ccctcagaaa |
| 98161 | tggggttggt tttcctctga ggaactgaga ccctcttcca agacatgcgg ttagcattag |
| 98221 | attgagtaaa tcgcagtcgc taccctaatt ctagagatgt aggaaacttc ctggggcgtg |
| 98281 | aagtgcctca ttccttcttc ttccaaggag taaaacccct tctcaatctg tccccagatt |
| 98341 | cccttttctc cccacacctt ctccgtggtc cgctactcaa agctctacag ttagagcttc |
| 98401 | atttttttta gtactcacga cRgatctatt acatttagta acctacccag tgctttcaca |
| 98461 | gtctctcttt tgatactctt aacagctctg tgagtcaagg atatttatct tcatttcaca |
| 98521 | gattggaaaa ctgaaaccca gagaaaaggg acttgcccaa ggccacacac caaattagtg |
| 98581 | acagagctag aactagactc cagttctccg acttccagct aagtctcttt agcttcagcc |
| 98641 | tactgcctct atagcggaag gacttgttcc aaggaacaag ttaaactgcc ccagggaaaa |
| 98701 | ggaacctgtg tggcctactt tgtattattt acacttcagc tggaatgaat ttcagatcct |
| 98761 | cccctgaaaS ttttaatggg gaaaaggaac tagagagact aagacaatga gacttgaaca |
| 98821 | tttttaacta aacaggaaac agaagggcta acacagcacc catgtctgaa gtgaccatga |
| 98881 | ttagctggtt tctagctggt gactagctgg ccctgactgg tccacttact accttaaagt |
| 98941 | tctcccaggt ttggtcctac gccctcttgc cactgcactc tatattctct tcttgggtac |
| 99001 | ccctccacac ccacaacttc attttccacc tgtatgcaaa tgtacacaag tgtgtatctc |
| 99061 | aagcccagcc ctcatctcta agctccagat ctatatagcc aattgcctca acatctcctt |
| 99121 | taggatacct caaaggcaac ccaaactcta catttctgaa attgaaacaa tctctgtgta |
| 99181 | tcccaacctt ttcctccaag cctggatctc ttccagtgct cctaccttta tccacaatat |
| 99241 | acccaagccc aaaacccaag ttagtacctc ttccatttac ttaatacctc ttccatttcc |
| 99301 | catctatcac taagtcttat cagctttact tcctgaatat cttttatata ggtcctagct |
| 99361 | accatcacat ccttcctgcc ttccctccca gccatatttg cgcactgcaa ccagagtgat |
| 99421 | ccttcaaaat gcaaagctga tactRttcac tctctacccc acccccMatt tttttttttt |
| 99481 | ctttttgaga cagagtttcg ctcttgctgc ccaggctgga gtgcaatggc gcaatctcgg |
| 99541 | ctcacagcaa cctccacctc ccggtttcaa gcaattctcc tgtctcagcc tcccaggtaa |
| 99601 | ctgggattac aggcatgcgc caccacaccc ggctaatttt atatttttag tagagacggg |
| 99661 | gtttctccat gttggtcagg ctggtctcga actcccgacc tcaggtgatc tgcccacctt |
| 99721 | ggcctcccaa agtgctgaga ttacaggcgt gagccgccac gcccagccag cccccattta |
| 99781 | atattaatac tatgtggttt cctattgctc ccatctctcc agcctccagt ctaaccatac |
| 99841 | atattcatcc ttctctcccc ttcagttata taacctttaa ggtcttcaaa atttccaagt |
| 99901 | acccttccac catagaacct ttgcatatag cagtcacatc tgcctggaat gctcattcca |
| 99961 | caccaccccc taccccataa gttaatttgt acttatcctt tagatttctc tcagtcactt |
| 100021 | cttcaggaat gtcttcctgt agactgttca gaccaggtca gatccctcaa ttacagcaac |
| 100081 | ctcatggccc tctcctttgt agtRttattt agttgcaatt Wttaatgtgt ttgttaagca |
| 100141 | cctactactt gccggcacta tgagatacaa ctgtaagaaa aaatatggtt tctgccctgc |
| 100201 | tggtgtttat ggacatacag atagtaagca gataattaYa gtgttacgag tgctaccatg |
| 100261 | gcaatcatag cactacaatt acagatttga ggataaaggt agacttctca gaaaagttga |
| 100321 | aacctgatct gagccctgaa ataagaatta gcaaagcaaa aggaggagag aaaaactata |
| 100381 | ttccaagcag acagagctgg aaggagacac tgttccactt agaggcccag tggcaggtgc |
| 100441 | agggaactta tggtgagtgg ttgcatttgg ctagactgca gaattggagt tgaggatgag |
| 100501 | atggtagtag aKgggggtgg acaggagcca ggttgaatct tggtcctcct tttggcacct |
| 100561 | gggaatataa cctcccagaa tatattagat tatgtggcag atttttcttc tttttctttt |
| 100621 | gacagggtct cgctctgttg cccagactgg agtgcagtgg cacagtcata gctcactgca |
| 100681 | gcctcgaact cctgggcttg agccatactc ctacctcagc ttcccaagta gctgggacta |
| 100741 | taggcgtgag ctgccatgcc tggctaaata tgtggcagat ttttcatagc agcctttgat |
| 100801 | tttttttcca ccatcagtcc atcatcagaa actctcgggc caggctgttg aatctcctat |
| 100861 | cagatttatg tgattattcc tcaaagcttc ctgtgttatc atgttgcaac ttgcctgaga |
| 100921 | gagggcagga ataggctagc aacaaatcaa attccaaatg ttagtaattt cccaaaactg |
| 100981 | aaaacagttt tctgtcaact tgggattggt taaataaatt aagtgaataa cattaaagta |
| 101041 | cgataYatag tataaaatac tttgcaacta ttaaaaaggg taatagtcta caacaatttg |
| 101101 | aaaattaaaa aataggttta caaaggaata ttgtttattt aaaaatatac ataatctagg |
| 101161 | ctgggcacag tggcttacac ctgtaatccc agcactttgg gaggccaagg tgagtgaata |
| 101221 | gcttgagccc aggagtttga gaccagccta agcaacatgg caaaacYgtg tctctacaaa |
| 101281 | aaatacaaaa attagcaggg tgtggtgacg tgtgcctgta gtcccagcta ctcaggaagc |
| 101341 | taaggtggtg ggattgcctc agcccaggag gcagaggttg cagtgagctg aggtagcacc |
| 101401 | attgcactcc agcctgcaca acagagcgag accttgtctc aaaaaaaaaa aaaaaaaaga |
| 101461 | aaaccgtgtg tgtgtgtgtg tgtgtgtgtg tgtgtgtgtg tgtgtgtgta aaaccttatt |
| 101521 | agctatacat tctaggttat attcccaaac tgttaatgga gattcaaact ttgagtttct |
| 101581 | atttgatgta cttcttgtca tttcacttta ttcatctatc atattagctt ttacagttaa |
| 101641 | aaaacaaaac agttaaagat actgtcaatt tggaaatgag aaattccagt tccgaaacaa |
| 101701 | ggcaggtatg cccctaaccc tgtcattcat caagttagtc agtctggaca gatgaaggtg |
| 101761 | ctggcatctg attacctcct ggtcataagc cagaatagca gatgggaaag actgactcca |
| 101821 | aaaacacagc cacaggagta agcatggcag catcaacact ttgtcagttc tccccagcag |
| 101881 | tcaagctctc tatgtgacac cttgaagact ccattaattc cttcatttct tctttcattt |
| 101941 | ttccattgtc catccatttt catttgtcca tcaaaccttt agtgaatatg gatttttttt |
| 102001 | ttttttttgg ccaggctcaa tgcagggtaa tgatgagaag ggagaaagaa gtctgagtct |
| 102061 | tgtccctgtc ctcaagctta tagggagtaa acaaagttgg ttataatgaa attaacagag |
| 102121 | aatacaactg cttccattta ttgagcacct gatgccagat gtacaatagt tctcaaggta |
| 102181 | gggtttgtct ccatttcaca gatgagtaaa caagctgtaa taaaattttg tttgaagtac |
| 102241 | agtgggggca tgcagaagtt agctattgcc gtgtaataaa ccatcccaaa acacagtggc |
| 102301 | ttaagataat aaacatttat tattgcttct aagtcttaga gtcagctgga gggttcttct |
| 102361 | gctttggctg ggcttactca tgtgtcggca gtcatctgtg ggttgggtga ctctcctgat |
| 102421 | cttgactggg ggtaggctga ctgtgggcta gtctaggatg gcttcagctg gtatctctgg |
| 102481 | ctctttYgca tgtctctcac ctacacatct cataaccctc cagctgatca gccatgttct |
| 102541 | catggcactg gcaaagagca agagggcagg cctcctttaa agcttctgtt tgcttcatgt |
| 102601 | ttatcgacat ctcattggct aaaacaagtc acatgtctgg tcccagcatc agagtgagat |
| 102661 | gggactacaa tgttataggc aaaaagagat ggatacaggg gagaccatga atagggatca |
| 102721 | tcaaaacaat cagcctaaca caggacacat taaccaattt tatctggaaa agaaaggctt |
| 102781 | tatagaagag gctacactag aactgagtgt tgaaacatga gagagtattc actaagtggg |
| 102841 | cagctgttac ttggaactgg atgcaggctg gtttaccaca atcaatagaa actcagcagg |
| 102901 | tctttctgtg ctcatgacag ggttctatta tttgtatctt ggcttcattt cttctggtca |
| 102961 | ggcacagagc agcaagcacW caacaaatgt ttgctgaatt gaattgaata tcttactgtg |
| 103021 | aacaaggtgt ggactgaaac agctgaacct gggaaccaat tgttagaaag aacagcttag |
| 103081 | catcaactct tctgaaacag aaatcctcag aattggcagt ttctcattca ttcatcccac |
| 103141 | aaacattttg agtgcctgtt gtaaacctgg ttctatgttg gataatgaat aagatgcgat |
| 103201 | actagctcac agcctgggct acagcttaaa tgcagaaggc taaaaattta accagcttct |
| 103261 | tggggctcca tttttaatac taatYcattc agaaaatgca acaggagctg gtgctacaca |
| 103321 | aaatcagaag tctttaggaa ctgcactggg ttgctctgNc aagaaaacta acagtaaaaa |
| 103381 | tctgccctta agtattttaa gttaaaatcc atgttcagtg gtgcataact ctggggtacc |
| 103441 | aacccagtat ttactttgag aaaagtatcc aatcttgctc aaaggtatca aatctgYtgt |
| 103501 | aggtgctgaa gtgggatgag cttctRtcta agctaggttc ctctagttct gtagtagaca |
| 103561 | gccaggttcc tctattttta caaaagaata atgtgttctt tgttgtctgc ctgtagattt |
| 103621 | aatatctaaa agattcaagt ctccctaatt gacaatccat gcccatggga ccagttcata |
| 103681 | acagaattaa gctgtgaagt gactcaatct gtcacctccc caaagctcag ctggtactgc |
| 103741 | tattttttca gaacaagaca ttcattttcc tctaaaccta ctaaacttga tccatgtata |
| 103801 | tgttaaattg ccaaaaaagg aatttacatg aactgtatta ggcttaaact gaattataag |
| 103861 | atatcaggtc cttggttttt aacagaaatt cttttttttt tttttttttt tttttttttc |
| 103921 | ctgagacagg gtctacaggg tcttgctctg tcgcccaggc tggagtgcag tggcacaatt |
| 103981 | atagctcact gcaacctcca acttctaggc tcaagcaatc caactgcctc agcctcccga |
| 104041 | gtaataaatt ccattttaaa gttgtttgcc aatttcattg tctctgcaaa ctaatttatc |
| 104101 | ctttgaatcc aatgatgaga actgcaatcc caaggtttct ctttttgctt tggctgccag |
| 104161 | gaatttcaga gctaaatgta ggcttagatt cttaaagtgc ctggcacatc tctctcttcc |
| 104221 | tgtaatctag tttctgatct ttctcttcca tttttaactt tctaatctgc actgtccagt |
| 104281 | atggtagcca ctagtcacgt acatttgaca ggtaggctag tccaaattgg gatgtgctgt |
| 104341 | aagtgtaaaa tatctcagta agttttaata ttgattacat attgaatgat tttttgatat |
| 104401 | attgggtaaa attaagttat taaaattaat ttcaccactt cattttactt attttggcta |
| 104461 | ctagaaaatt ttaaattata tatatggctt gcatttgtga cattatattt ctagtggatg |
| 104521 | gcactattct gcaaatcatc tgttttatga ctgtgttatt attgtaaacc tgaaatcctt |
| 104581 | ttggaaagca ggtgaagtaa tacaaataat aatacaaagg tggcagcagt ggatggaatg |
| 104641 | agaaagggga ggtcgactgg gRcaagttct tgtccttatc cttgtcaatt tgtcttttgc |
| 104701 | ttgtgtgatt cacatacaaa ccccttggta aagtgtgttg tcagctctct tcatcccagg |
| 104761 | tgagtagtat tctcatggct ctcccttgtc tccagaaaaa cctcagcttt actttagcta |
| 104821 | acaaattgga aacgatctga taagacaatt cacaccacat tgcaagtgac tgataacaac |
| 104881 | attgtgttca tccctgtcag ggagtatttc cattttaacc ggaaacaatc cctgaaccca |
| 104941 | caggaatgaa tgcctaatgg tggagtttca gccatcagtg acaggtgagt gagaggtaga |
| 105001 | ctctggtttt gtgtgtgtgt atgtttatat gtgtatggct acattcagga tagggctgaa |
| 105061 | agtagggtat ttggatggaa aggtaatggg gctggcagag aagccaagcg gagaaggtgg |
| 105121 | caaatcccca gctgggccag actgagaggg gtggtgagac accaaccaga tgcggtgcag |
| 105181 | gccctcaagg atttgttaag caatagaata gcccagttga tgtcaMcaga gggtgggtat |
| 105241 | caagacagtc tccacagtaa ctactgtaga gtaacagaaa aggtctcaga gtaggattca |
| 105301 | ggagattatg actgcagtct acaactgact cactggctta actctgggca agtcctttta |
| 105361 | ccccttgggt ttcagtttct tcatctggca ttttccagac aaagaaatgg gtgttcgatt |
| 105421 | ctcaaatgag ttctaagaga tgtgaaggtg ctctgagaag tagaaagtgc tctatacatg |
| 105481 | caaggatgca aggaagagtg atttcatatc ttaaaatgga aatacagcca ggcaggccta |
| 105541 | gagtagtaga ggacagaaag cagggatgga ggactatggg gaaacctgaa agccggtgta |
| 105601 | cttgaaacac tggtctttct ctgtcctccc aggctgaacc cagactccca gggcacctgc |
| 105661 | ttgcaccttt gaatgatggc ctgaactatg aacaaacggg actatatgaa cacttcggta |
| 105721 | caggagcccc ctcttgacta ctccttcaga agcatccacg tcattcaagg tcagccccca |
| 105781 | gagcacagta ctccactcac taaggcagca ggcccagtgt ggtccctagg aagaaaccat |
| 105841 | aatctactta aaagcctaaa gagattcttc ccatatgctt gcagatgtgt tgttactgtc |
| 105901 | ttttggaggg taggggagag tgagaggttt ttttctgtct ttttgtttgW tttgattttg |
| 105961 | ccccgataca gccccaggag atcctaagaa catgtgcccc tgtttgtttt ttttaagcag |
| 106021 | caaatctgaa aggaaacagg gggctatcac agtagttgat catctttaaa agaagccagg |
| 106081 | ctacagcagg aaagtcaagt ggaagaaaac tgggaagcca gtctatcagc caatagtttt |
| 106141 | attctcctgt ttttacctga agagaggagg aaaacacagg ctcctgtgga gtcccagcaa |
| 106201 | gtaggcaaaa cctttctcct ctgtctgcag acacaagcct aatctgactg gccaaacatg |
| 106261 | aggggtctta tctacaaact ggcatatttc cagtttaaat acttactgac aaatttctta |
| 106321 | cactcattcc agccaaatcc aagtttattc catcatcacc tacacttatt gcaggttcca |
| 106381 | acactgtcca cactcgctcc cattttgcca ttcaatgtct ttgtaaatgg gtagctagtc |
| 106441 | ccttcaggag ggaacatagg gaggctgtca gccatgtcat attcccaatg agtcatccgt |
| 106501 | tcttcctagg gaagttctgc catccctggc ttttgttatc tggtctttca ttaaagcatc |
| 106561 | agtccatctg tcctcacaga atacacgtta acatccacta tctcagaact attatttggc |
| 106621 | tttagtcttc tctcaggcaa ctggtagcca ctgcctttaa gctcgggaag ctggcaaata |
| 106681 | gcaccagtct tagattgggt ttagggtcta cttgggttat catcagccac agtggtaact |
| 106741 | cacgtttgtt ctctcacgag cacacagcca cctgctgtga tctttattgt gcactgcatc |
| 106801 | acttctgatg taatccatgt ttcacaaggg gttttagctg caatagtaac tatctctaag |
| 106861 | acaggtgcaa tgacagaggt gggaaggaag tatacctcct acctatcatg ggaatttcta |
| 106921 | actctgatgg aggaggtgta gctctgccct ctatggagtc ctcagaattg ggagagagac |
| 106981 | aagcagtacc aagggtaact ctacatcaca aaacgtaaat ggtacagatg gcccatcctt |
| 107041 | cattcaaccg ttcaatattt actgagcact tactaaaagc atttatatat gtgtgtgtgt |
| 107101 | gtgtgcgtca tatgtatatg ccaggtattg tgctccttgc taggtatgtg aggcacatca |
| 107161 | aaggcccaaa aggtcagagt cccttcagat tccccagagc ttctgaaaca cttatttaca |
| 107221 | gctggggtta agggatacaa tagagtttgg gggtacctgg cacactattt ggtgaatgaa |
| 107281 | taaatattac agaatctgaa acggcagtgc ctttggaagg catctaatct gtgctaccat |
| 107341 | caacccagtt cctctaactt ttcacaaacc tagagtgtta aggaagtccc ccaccatata |
| 107401 | cacctaccaa caccttccag ttggtggttg agggatgaca cagggaagtt caatcaaact |
| 107461 | aacttgtatt ctgacacatg gcaagagcta catggcagcc tttcagagag aggaaaacag |
| 107521 | gaacatagtt tcccagatct tcctggagct aaatgctccc cagttcccaa agcattgata |
| 107581 | tttgtggtcc agcttccaga cctgctttgg cctactctga acaaggaact ttacagattc |
| 107641 | ttaggcagtg tagtgcattg gtggaatctg agaggagcag caacctgtgg cagcctggat |
| 107701 | ataccactcc atccttgRta agagcagctc cagccagcat caagttttag gcaactctgt |
| 107761 | cccaccagtg attccctctg agcttggcct ccaccccatg cttggtggca ttccccaagc |
| 107821 | tgctacctta tcacatgggg cactagttca tgtggggtta ttttgacctt aattttgatt |
| 107881 | catctgtgtc attctgtttc agggttctMa ggaggtRtgg ggagggaagc aggattagga |
| 107941 | attcatgagc tggggtacgc cgttccagta ccctcctgca accccaggcc ccaattgtgg |
| 108001 | gtcctgctca ctctaggatt aaggcagaga gccatctggt ggatgttgcc tgacctgcat |
| 108061 | ctgaggagga ggagaggtgg gggtggaagg gcaaagggtg tccttccagt agagagcaga |
| 108121 | agagagtgaa gaaagctggc tagcttccgc aaatactaaa tacttccttc cccaggggtg |
| 108181 | aactagggaa ccaaaggctt agcagcaatg ggaactggac ctcattttga aaaagtggga |
| 108241 | gtgaggcttt ggggtgttcc atacaccggg gttctaacat aatcctattc cctattctct |
| 108301 | ctcctcctca gccaaacttt ctgaggatgt tgtttatgct aactgcttat atccattcac |
| 108361 | tctttttttg gtagagacgg ggtttcatga tgttgcccag gctggtctca aacgcctagg |
| 108421 | ctcaagcaat cctcctgtct tggcctccca aagtgctggg attacaggca tgaaaccagc |
| 108481 | ctccattcac tcttgaactc actcacttcM atctggcttc tgcctccagg cccttgccat |
| 108541 | tccactgaag cagcccttac caaggttacc agggacttct aagttgccaa aattactttt |
| 108601 | cagtccttac ctccctggaa cttactacta taaactattt actccctgaa catctttaaa |
| 108661 | ctgtcctctc ctgattttcc ctgatcattt aacttctcta atggattctt cttcctccct |
| 108721 | gaccttcctc cctaccctca ctctccctga ggaatctcat ctctttccct aacttgtatg |
| 108781 | ccttctatgc taatgattct caagtctgtg tctcctgagc ttccgacctt catctccaac |
| 108841 | tgcctacttg acttctccac ctttttgtac aggaacctca aatacaccaa agctaagcct |
| 108901 | gaactcatca cattcccaat gctatttctt cttctgtaat tccttaccag ttaatacaac |
| 108961 | tggcaaccac cctttaatat tcttctacct ttacatccag tcaaccaaat tatgttgatt |
| 109021 | gtagggccct ctcatgtctt gcttggatga ctcaaacagc caaattggtc atttggtcca |
| 109081 | actcactccc taaaatccat attccacact gacacttaag agtgatcttt ctgaaataaa |
| 109141 | agatttgaat catttgcttt aaacactcca gtgccttccc attacacaca gggtaaagcc |
| 109201 | caagatcctt agcctgacat ttgagacttt tgactggatt cctgcctcct tttcctctct |
| 109261 | gtcccccttg tgcagtgcgt gcaagcactt gtgcaggcac acaaacccgc cccagactcc |
| 109321 | ctggctgttc tgtacctcta tacctttctt catgctgttc cctgcctgtt tacccttctt |
| 109381 | tgcctgataa acatccccta atccatggag attcagctca ggcttcacct tccctgggaa |
| 109441 | acttccctga gctcccatac tgggtcaata gcctctcttc tgtgctgcta caactattat |
| 109501 | atagcactta tcacaccatR ttgccagctt gctcccatct tgcccatata agctcctcca |
| 109561 | aggcacaaat gtctgctctc tgccctatta ttcatacaaa gtctggaaca gactgtctct |
| 109621 | cattttgttt atcactgtat atccagtacc tggcatactg taggtactca acattMatgg |
| 109681 | aaagagagac aggatatcaa agtgtattcc aYtgaagtga attctatagg ctttaccagt |
| 109741 | agttaattca ggattttgtg ctatcatcta ctcagctcaa gcccaacaat tctggttctt |
| 109801 | agttttcctc aaccccacac tggtttggta taaaaaagac ccagtggtac agtggacatg |
| 109861 | agaagtccat gtgacctgga aagccaagag cttgagatag aagtctctgg cccttagtct |
| 109921 | aaacttactg attgctggaa gctgtgggac agggttgcac tagactcagg cacagggcta |
| 109981 | tggcccttcc agaccctcca gctacagcct ggatggctaa tctttccagc tctgtccctt |
| 110041 | cccaccccct aaagccaaaa aagcaaagat Ratacccaag tctcactttg tccccacaga |
| 110101 | tctggtaaat gaggagccaa ggacaggact acgaccactg aagcgttcaa agtcggggaa |
| 110161 | atcactgacc cagtccctgt ggctgaataa caatgttctc aatgatctga gagacttcaa |
| 110221 | ccaggtggct tcacagctgt tggagcaccc agagaacctg gcctggatcg acctgtcctt |
| 110281 | taatgacctg acttccattg accctgtgag ttcctaacag taggaatcca gtcaggggag |
| 110341 | cctgaagcca ctttgttcag cccccaacct ctccactcct acatgttatc aaggaagtaa |
| 110401 | tgaggcagca tggtacagtc caaagcacaa ggctaggggg ccagacagat agatgtaggt |
| 110461 | tcaaatcctg ggtttgcctc ttactgactg tgactttgag caagctacct aacctctcaa |
| 110521 | aactgcagcc tccctgtcgg aaaaatgggg ataatatgcc agtatcataa aggttttgtg |
| 110581 | aggattgaat gaaaatacat gaaaaccacY gggcacagcg gctcacgcct gtaatcccag |
| 110641 | cactttggga ggccgaggtg ggtggatcac aaggtcaaga gagatcaaga ccatcctggc |
| 110701 | caacatggtg aaaccccgcc tctactaaaa atacaaaaaa ttagctgggc gtggtgRYgg |
| 110761 | gcgcctgtag tcccagctac tcgggaggct gaggcaggag aatcacttga acccaggagg |
| 110821 | cagaggttgc agtgagccga gattgcgcca ctgtactcca gcctggcaac agagtgagac |
| 110881 | tccgtctcaa aaaaaaaaaa aaagaaaaga aaatacatga aaMccaacaa acaactggta |
| 110941 | catcagaggc tgaagtaaaa ttStaactgt cactgcgcac ccactgtgac ctgtgctggg |
| 111001 | ttctcctgtg gaagaggtag tcaataaagt acagcactta ctttctgtga actcagattc |
| 111061 | aagttttaag aaataaagtt tggccttttc tagtccaaga tccctaagag gggaaaggag |
| 111121 | ctggagcatg acttggacac tattcccctg gtgtggtgat ctcaatttaa ccagtatttt |
| 111181 | aaaagcttct tatgtgccta gcatgatgct ctgtcttcct gttgcttgct gtgatagaat |
| 111241 | cgtaKatcac caaagctgga aaggtcttca gtgaaggggc aactaaggct gagagaggca |
| 111301 | agtcagtggc agtgctggta cccaggccca cactcctgac cctcagatca cagctccttt |
| 111361 | tcagtgtgat gccctgtatg tcttaaaaca taaataaaag taggttacag tcatacagta |
| 111421 | tggctgtgga gggttgRagt ggagggcagg ttgttcccca cattcttcct tctctgccca |
| 111481 | cgagtcagcc ctgagcagag attcagggtc ctgtcctgtc tagcctggct tttggtttcc |
| 111541 | ctccccaaca ggtcctaaca actttcttca acctgagtgt cctctatctt cacggcaaca |
| 111601 | gcatccagcg cctgggggag gtgaataagc tggctgtcct tcctcggctc cgtagcctga |
| 111661 | cactccatgg gaaccccatg gaggaagaga aagggtatag gtaagtgccc tgcccctgga |
| 111721 | ggtagcgtct agctgggctc ccctaaagga aaggaggagg agaaacaaga aatctacgac |
| 111781 | cattcttctg ccatgcttgg acctgggaag ggagtaacag acctctaagc attccttgct |
| 111841 | ctcagtcagg ataaacctaa tctttcccaa actatgctca agtccctcca ttccttgcag |
| 111901 | tcagggaaga caacagcaac tagtggaggg ggaaggggat tctggtgggg gtgctggccc |
| 111961 | ccagcctaag tcttccccac aggcaatatg tgctgtgcac cctgtcccgt atcaccacgt |
| 112021 | tcgacttcag tggggtcacc aaagcagacc gcaccacagc tgaagtctgg aaacgcatga |
| 112081 | acatcaagcc caagaaggcc tggaccaagc agaatacact ttgaggctcc cacgacccta |
| 112141 | gtagtcctaa aggcctaagc atagacagca tggKttgaca ataaataatt tgagctgttg |
| 112201 | agcagatgag aagtcactta caccttgtag agatgttctc taactcaggc aactgcaagt |
| 112261 | agctctagcc ttttcttttc tttttttttt ttttgagacg gagtctcact ctgtcacaca |
| 112321 | ggctggagtg cagtggcacg atcttggctc actgcaacct cagcctccca ggttcaagag |
| 112381 | attctcctgc ctcagcctcc ccagtagctg tgattaaagg cgcctgccac catgactggc |
| 112441 | taattttgtt gttgttgttg ttgttttgag acggtttcac tcgtcacccc agctggagtg |
| 112501 | cagtggtgcg atctcagctc actgcaacct ccgcctgctg ggttcaagcg attctcctgc |
| 112561 | ctcagcctcc tgagtagctg ggattacagg cgcctgctac cacatccagc taattttttt |
| 112621 | ttttgagacg gagtctcgct ctgtcRccca ggctggagtg cagtggcacg atctcggctc |
| 112681 | actgcaaact ccgccttcca ggttcacgcc attctcctgc ctcagcctcc caagtagctg |
| 112741 | ggactatagg cacctactac cacgcctggc taattttttg tatKtttagt agagacgggg |
| 112801 | tttcaccatg ttagccagga tggtctccat ctcctgacct tgtgatccac ccacctcggc |
| 112861 | ttcccccagc taatttttta tattttagta gagacaaggt ttcgccatgt tggccaggct |
| 112921 | ggtcttgaac tcctgacctc aggtgatccg tctgctttgg cctcccaaag tgctgggatt |
| 112981 | agaagcgtga gccaccacgc ccagcctttt tttgtatttt tagtagagat ggggtttcgc |
| 113041 | catgttggcc aggctggtct caaactcctg acctcaggtg atctgcccac ctcagcctcc |
| 113101 | caaagtgcta ggattacagg ataagccact gcacctggcc agctctagtc ttattttgct |
| 113161 | gaaaaaggga ggcaattgag ggaaaggcct gctggcctgg gacactggag acgtgggttt |
| 113221 | accacacagc cttgggaaat ttatctaact ctctgggccc ctttgtactt ttcaRcttag |
| 113281 | agatttgggg gttaaagtag atcagtaatt tccaaactct tcacagagta ccagggtctg |
| 113341 | tagagatgcc tcacaggcct ccatgacagg ccaagaagat ggcctatgat ctctgaaacc |
| 113401 | agcccccact tcaatcagat caaagtaatt ccattctgtt ttatatgctg ggattctgct |
| 113461 | taagatttca tttgataaaa agaatcttct gcttaaaaac atttgcttgc tgactccata |
| 113521 | ttaggtattc ccagaagagc ctggtctgct ttctctctcc accccccaac ccgccatgtc |
| 113581 | tcccttcacc cccaggggct gttggtaatt ccaYtgagtc tgaggcaggg cccccaagaa |
| 113641 | tcaaagctgc ctcagattca ggtctgttca gctttgacag tgttcccatc atcactggac |
| 113701 | aagggacaag aacagtctca tccagtctga ggttagccca agccaggtct cagctaagag |
| 113761 | ctgacaccct aaacccttgt ctcttgggtt catcttcaat atccctggaa gatgtcccct |
| 113821 | tccccccatc ttactctcat agacctaagt aatcaaaagt agaatgggta gaacagaggg |
| 113881 | gccagaacca gcacatggat gaatatactt cctttatcga gggtgacaaa ccaaaacaaa |
| 113941 | aaaagaccaa acatgtaaaa acccMgggtt ctagaaatac aaactcaatt cattcaaatt |
| 114001 | caagctcatc cagaccctgg tcacaaaccc tagtgaggtg catgtgagca ccaagtcagg |
| 114061 | gagagggggc aggagtgact ctgaggccaa cagaRagggt gggaagggga tctccctagg |
| 114121 | tcccYtggtg atcacccccc acccaaactg gtatctccac attctcaatg actatgaaga |
| 114181 | cataccaggc tctgtccttt tggcccccac ccYatccctg gccagagctt caaaggccag |
| 114241 | tcccaaaagg ttctaccctc acttgctcag gcttctaRcc ttcctcttct cctccttctt |
| 114301 | cacatttttc tctgtgatta Stagcaggtt agggtactgt ataagccgca gtgaggctgg |
| 114361 | gggccaaggg ggtggggtag agatgggatg aagagaggag aagagctgtc caaggacccc |
| 114421 | tctcttcatg ggatcccaaa ctgtacaacc agctcctctt ttgcRtccac acggatctga |
| 114481 | gaaagtgcac tgtaggcata agcagctatc ctggagacct gagacagaga ggggccgggg |
| 114541 | aggagaggaa cagagacagg tgaggagggc aatcaggaaa aatggggagg ggagattaga |
| 114601 | tggtgatgga gaaggaggga atgaagggga acaggaaggc aaagagaaag acagggatgg |
| 114661 | gagagacaga aggaaaagaa caaaggggag tgaaacaata aaggttgcag gaaggagata |
| 114721 | ggaaagacat aaaaagttaa gagaacgagg tgggagggag ctggggccta caaggctacc |
| 114781 | aggctcccta actccacctt ccctacctcc tctgaggcag agtgggtgaa ccctctgcca |
| 114841 | taccctgggc agtgtgagaa gggtgggcag gggcaggtga ggggtgtctc acctgctgca |
| 114901 | aatcagagaa cgggatgggc tcactggcca gcacttggtg gggctggctg gtaagagacg |
| 114961 | gcagcggtgg cagcttcttc caatgggtca ggctgctgct cagcacagcc aagcgggtgc |
| 115021 | tgaccaagag agagggggtg ggggtaggca gttaagcYac agtctgccct gcccagccca |
| 115081 | ggtaagcctc atcccctcag gccagagaag cagggtgtcg aggaggggta tctgggaggg |
| 115141 | aaggtctcag ctcatcacag actcaatcta ggcccatctc ccttggcctc agYgccctca |
| 115201 | agagtgtcac agggtaaaca gggctgacat gaataactac tgaagcaccc aagtgatctg |
| 115261 | tggtcagtct ccagggctgc accaaatgag aagcttggac taggggccag gctccaaagc |
| 115321 | tggaacagtg atggagggtg ggagcccaag tagcctgagg gacccacgct ggccaggtgc |
| 115381 | tcacctgtac tgcctggcac ggtccatgta ctcatgctgc tccatgccct gtRagtctgc |
| 115441 | agcagacaca tcaatgatgt tgctatgggg agaggagaca gagatgacat gacaggtgct |
| 115501 | tccgagaagg acaaacagga ctcagtgcca ggtaccaact ccagggctat ctgacaccag |
| 115561 | tttccctcct gcctactgga gcagggctgg acatggaggc acctctcccc agtgaccttg |
| 115621 | tcccatcccc catgtcctga gcctggctcc ttctatccta gcctttaata tggatagagg |
| 115681 | agctctgacc caggcctggt cctctgacac ttacctggct gtcttggcaa ggatggaaga |
| 115741 | gagcagggcc tgctcatcag tgcgagcgga aggcaggctg tggtagttgg gctcggctcc |
| 115801 | attgagagct ttggtagggg ggctgctagg gtccagcagc agcttccgct cctctcggtc |
| 115861 | ctagaagtgg tcatgggaga gaagtcagcc ccaggacccg tagatcctgc ctttcacggg |
| 115921 | tgctaccctt agcccagctc tgaccttaaa caccaaaaat gtttctataa ggaatatcta |
| 115981 | aagtgcccag ctttatggaa tgccagataa gactcagttc ctgcctatcc ttgggagctc |
| 116041 | aactgagttg gtgttctagg ataaatgaga gtggaaccag gcagtcctag aggccctgaa |
| 116101 | aaaatggaca gaggcacaaa gcaggtagtc agcaacagcc ctggagcttg aaaaacattc |
| 116161 | taggacctgg caaagaaagg atactagagg ctggaagaca gWggagctga gacatagcaa |
| 116221 | gcactgggat ctaaagggga aatcagtgca agtcaagagc ccaagaacag ccttcctcat |
| 116281 | cccctaggac caggccttcc ccagcaaagc tgttcgcttc caagatgRaa gccctggcga |
| 116341 | gtccagacaa agccctaggc cccaagagaa agcaagccca acctctccat ggccatttct |
| 116401 | caaattatgt cagggcagga atcccagccc agctctgggt cacacagtac aagtgtgttc |
| 116461 | ccttcttgaa ggcaggtccc ttcgaaggtg ttcaccctcc tgtgctctgg tgctcttttt |
| 116521 | gtacgtgact cccagaacca aactgagtgt acatggtgta tcctgatctg agcagtacag |
| 116581 | ctgtagccat gtatggtgat tcacaccagt aatcccaaca ctttgggagc ccaaggcaga |
| 116641 | aggattgctt gagtccagga gtttgagacc agcctgggca acaaagcaag acctcgtctt |
| 116701 | tgcaaaaaaa aaaaattagc tggacttggt agtgtgagcc ctgcagtccc acctacttgg |
| 116761 | gaggctgaag tgcaacgact gtatgaaccc agggtttgag gctgcagtga gYtaggactg |
| 116821 | tgccactgta ctccagtctg ggcaagagag caagacccca tctcaaaaca aacaacaatg |
| 116881 | gcgctgttcc ctcccttgct aaaccacctg agtagcccat gtaaattttc agcagccctg |
| 116941 | ttcctgctgg cctgtgctga taatcacaac ccctgaacta atgtaagacc aaaccaagct |
| 117001 | tcctcattgg tttggtcttc ctccttgtcc tctttcagtc ccctgttcta tctactgtaa |
| 117061 | tatttctgga tttgaccctg ccttgagacc atcagctgca aatccttgct tgctgtatca |
| 117121 | gcagtaaatg agaaaaacag acctgctggg tatctatcca acccataaag agcaagagtg |
| 117181 | ccctgaacaa agccaaggat Rggcccaggg ggacatcctg gctaatgctg actcaccaac |
| 117241 | cttcagtgcc agactcctca atgctcccct ccaggcctat ctcagttcct cagtctttcc |
| 117301 | tgctgcaggc cgcagctccc ctgcccacaa ctccacaact ctccttgtag agggctactt |
| 117361 | cctcctgccc tgcttaggga ggcccagcct tttcacctta ctcttctttc tcaaggctag |
| 117421 | gttgcccaag tgtcctcagg caatttgatc cagttcccaa cagcaagaga cagaagctgt |
| 117481 | gggctttgcc ccatcacctc tataaactcc cagacaccct tcctccctaa atacataggc |
| 117541 | tcagtattaa agagctaaaa agacNaaatg tttgccaaag ataaaactat gtatttctaa |
| 117601 | ggaaagctgg ctccataatg aggcccaagt tagcttccta tgtgtctgtc ctcccagctc |
| 117661 | cacttcagac agatcttcct gcaggacagg gtagtctctt ccctatcagg ggttcttcaa |
| 117721 | gaagaaggtt ggctgatcac ttctcagcct tcagctaaga tcaagtgaac aacaaggtta |
| 117781 | ttttcttgaa acccagactt aaagaagagg ccatgctacc tacttcagag cagacaaaca |
| 117841 | tcaccccact cccaaagtgc gagtggagca tgggttaggt ttgtcacatc aaggtttgtc |
| 117901 | tctgcattag tgtaacccaa gaggcagggt ttcactgtct accttagcac agacatagca |
| 117961 | gcctgcccRt gactggccac ctggtctagg ggcatggctg agacagcttg gccccatgga |
| 118021 | taaagtacat ggcctagcgt cagaacacct gggcactagc acttctcagc ttggccaaat |
| 118081 | cacttagctt ctctgaactt cttcctcacc tttcaataga cataacccac actaatatga |
| 118141 | cggatgtctR gcaagagtca aattagataa ctgtgaaggc cctttgtaaa ctgtaacgtg |
| 118201 | ttgtatattt tttattatga taaagagaca gagatgagag tcggagaggg gcctgcccaa |
| 118261 | gaactcaaag agggactgtg ggatggtgga tggttatggc ccttgctaca tgaggcctca |
| 118321 | aatcacccag cagtgattaa gcaccttctc tgggtgttag gaacagaaat gagagccccc |
| 118381 | caaggagaag gtagagattc aggagagcag caggcccagc tctcccttcc aagtagaagc |
| 118441 | ttgtatcctt tctcatacgg cattaatcct tcttgactca ttccctgcaa gagtccatat |
| 118501 | tatgctttaa gggacaattt cccactaggc tacagctctc ccagtaggca gggttattgt |
| 118561 | tgtctctttc tcaaaaaggt ggaagtgaag tgagaggtat taggcacaac acatgcctaa |
| 118621 | cacagggcaa aggaaatatg tgttcattgg gtgtatggag aattgagaac cagaagccac |
| 118681 | ccttgcttcc catacaaacg acattttcct aggcttaagc ccttaatcca aaatacgttc |
| 118741 | actgaagaac gaactacttc ccaagtacca gtgatataat ctatttttgt gaccatcttc |
| 118801 | cccattagac cgtgagctac ctgaaacctt tctactctct caggtctact atatgttcaa |
| 118861 | atgtagaatc cgggcttaga gacgatcaaa aaaggagagt agacagcaac ttgagacttc |
| 118921 | atccttgaat gtcaggccag gcagggcagg gattagagtg tggggagaag tgagaatgca |
| 118981 | ctaagaggaa gagggagaat ttaaaactgt acaggtctcc ccagcctaac cctttctcca |
| 119041 | aaggtatcag atctacagcc aaattctcag aataggcatg tctccagcta aaggaaccct |
| 119101 | caggccccag gcagactgca ctgaggcaag ccaaactcag aaccaaatga acttcccgct |
| 119161 | ccatttctct tttcccacaa tctagccaaa gctaaatcct ctttgttgct tcccagacaa |
| 119221 | tttcccacat gtcccgatgt cctcactgta tcgattaaat ccttcattgg ccaattcaaa |
| 119281 | ggccaccaac ttccagagtc gtcccctggc cctagactag cagcgacctt ccttctgcaa |
| 119341 | ccccagggac taagacgata cacgaattcc tgattcatct caggacagac taactcaggg |
| 119401 | cctcagctta cccaccccta aaatggattc gcaaagccca ctaatacaca gaagacacag |
| 119461 | gaagctgggg aagggagtgt ttcccgggga cctagactga ggatccttga atctgaaaag |
| 119521 | ttttgaaggt aggagcaaac ggaagtggtg ggagcgagcc ggacaggcgg ctgcctgcac |
| 119581 | accccgaggc ggggactgtg tagaaatctg gggctttaac aggggtagga gcccggcttg |
| 119641 | gcgtctcctc tgtaatctgt cccctccagg cctccgtatt cagtgagccc tgcagtccgg |
| 119701 | ctgcccgtcc tccgcaggtt tctttctggg ccccatgccc cagtgtctta gccctcattc |
| 119761 | ccgagccccg cctgccgcgg ccgcacctgg tccgagtcct cgttctcgct gctgtagcag |
| 119821 | caccccatgg ccggggtcgg gccgggcgct caggccgcgc cgaggaggga cggcgtccgt |
| 119881 | gaggagccct tccggtcaca tgacccgcgg cggcctcccg caggcaacca cccgccccca |
| 119941 | ccccccgtac cccctgtccc ggagccgctt ggccgcctca gtcaataccc cggcttccgg |
| 120001 | tcccgcccgc tagcgcgtga tcgttgtcaa ccaatgggaa ggcgtaaccg tacacgcaag |
| 120061 | cacgtgatgc ggggaaaggg cggggcggat cacatgactt catctttcca gtctctaaac |
| 120121 | tggcagtagt ggccatcgtg aattgtgtta tgcgttctcg tggccactag gccacttttc |
| 120181 | tgggccattg agcgaactcg taccaaatgc cctcgtatgc ccttgtgata tactcttcta |
| 120241 | aatgccatgc gaggggcgac tgggatccag gcctgatgct ccagatcgta cggtctgggt |
| 120301 | gcacacacac agatgatcca gatgaccagt acctagtcgg ggaacacaga gggtgtatcg |
| 120361 | cccagctggg ctgggggtga atacacagag ggtgtattct ccacctgggc tggggatctg |
| 120421 | cgacttacag gaagaaggga ccttatacta ccttcacatg ctgacgcgca gcggagataa |
| 120481 | atcctggtag aggagataga agccacctca ctgacctctc tgctttcagg cttttatctt |
| 120541 | taattctgcc ttttttaaca gcacccattg taatcgaacg cacaaagctg accatcagtt |
| 120601 | ctcttttcct aacccccgct ccatgtcttc ccttgggccc tgaacacagg tccaagtccg |
| 120661 | tccaagattg tatagtgatc catttgttgt tcatgagcgt gtgattgggt gttcaccagc |
| 120721 | atgtatcaaa tgtgccaccc tcaaatcttg ttttggcaca ttacccgtct gacataacaa |
| 120781 | gagcctgaca cctctcccac tgcatgttct ggggtgaggg gtggaggggt cagcaatgtg |
| 120841 | tttcactgga catagagaaa taatgtagtt tcctgattga tctactggac tttgtacatg |
| 120901 | cagcctggtc tgcctactgt ctttactgtc ttcgagactc agtacaatgt cctcctgatt |
| 120961 | accttctctg actccattct ctggacatgc ccagctccct atgcttaatc ctcatccagt |
| 121021 | gttaattcat gcatttattg aatcaacaaa tatttgctga gcgtctacta tgtgccacac |
| 121081 | attgttatat ctggatatgc agcagttcac aaaacagcca aagtccctgt agtcatggaa |
| 121141 | catccaatag tgatcacact atattgtatt ctctgttcct gggtcagtct cacctaccaa |
| 121201 | acttgggagt tcctggagca cagtgacagg ttctgactct tccctgtagc ccaaggcatg |
| 121261 | gccttgctta cagaaaactt gcatatcaat gagcatgtta cagtcacccc atcaccttgg |
| 121321 | ctcaccccat gccccatctt ctgatccaaa gtattggaga agtcaccaga tctcatacct |
| 121381 | cttataaccc ctcaaatcaa gaccctgagg ttctgtaaat cttttgaggt tcctgaaggg |
| 121441 | taggaggcag ctcaagctcg tccctggggc tggtaacttg aactgcaggt ctcaggtgac |
| 121501 | agcagcagcc tcagcatgaa taattgaaag ccattccagc ctcctacctc agggttatcc |
| 121561 | cccgcagcag gccctggact ccccgacctg tcctgtgctc tggtcctcca gcccaggtaa |
| 121621 | gcagggccct ggattgtggg ttcccaagct caccagttca agcccaccgc atcactcacc |
| 121681 | tattcacacc catccataca ctctttcatt tttactcccc gccatcacca ggggccttgc |
| 121741 | aagaaggccg gcaaggaggt cagatctgga tgaggaggag aatggtccag gacatgggag |
| 121801 | gagaggccca ttccccatag gtccctggag agaagagctg agtagaggga aaatgtgtgc |
| 121861 | tgtgtgttat gtgtatgtac acatttgtga agggaattct gagacagcac aggagaagag |
| 121921 | gggaatacag cggagaggag atggggctgg agaattatta aagaatcaaa gactcctccc |
| 121981 | atcctcaggg aagagcaggc agagggagag tcctggggtg ggaaagagga catctcactc |
| 122041 | taacacccta tggggtattt tgattgcatt ttactgaggc agggcacagt gatagtgaag |
| 122101 | gtgtttttga ctgcaaatct atgctctttt gctacacagc agtggtcaga ggagaccagg |
| 122161 | caggcagagg gtaggggtgg gggaagatat cccagaggtg aattagtgaa aggggtcctg |
| 122221 | aagaaggggt gacttcaagg ataaagagaa acaagtcagg ggaacaatgt gaagatggga |
| 122281 | ccaggggttg ggaccgggaa gaggtggttt ggggctgggt gctgaaagta gacagtatag |
| 122341 | agtccttgaa agtacacagg cttggaatca cactaacctg gattcaaatc ccagttctgc |
| 122401 | tctgtgactc tggacaaaag acttagcctt tctgagccgt ggtttgtgaa atataaggat |
| 122461 | aataattgct actggcaaaa gctacacaaa taggcaaatt gtgggtatgg gattccctcc |
| 122521 | ctacctccct ccaccccagg gcccaggtag ggaccatgtc ccctgccatt gcattggcct |
| 122581 | tcctgccact ggtggtaaca ttgctggtgc ggtaccggca ctacttccga ttgctggtgc |
| 122641 | gcacggtctt gctgcgaagc ctccgagact gcctgtcagg gctgcggatc gaggagcggg |
| 122701 | ccttcagcta cgtgctcacc catgccctgc ccggtgaccc tggtcacatc ctcaccaccc |
| 122761 | tggaccactg gagcagccgc tgcgagtact tgagccacat ggggcctgtc aaaggtcagt |
| 122821 | gttccctagc cttctgctcc aagaagtacc cccaagacag tgaaggaata tttgggatct |
| 122881 | gttgctgctt aactggatga attgggcagg ttcttgatcc tcttttaggg cctctttttt |
| 122941 | ttctcatctg gaaatgagga gcttggacta agtcatttat tcagcaaaca tttattgcca |
| 123001 | cctcttttgt attaggaatg tgctaggtgc cagggagagg ggtagaggcc acagagatga |
| 123061 | atgagccaca aatgctggcc tcaaaggaac cataatacac agtggaataa ccatctcagc |
| 123121 | agataaccca tagctttgta attatctgtc tccacatctt tcctctccac catggaggct |
| 123181 | tccacatacc atttagggta gctgtctgcc tggatttatc tcaatcccag cataagtgct |
| 123241 | tttagggtct tatttaagaa attcggctgt gtggtggctg gtgcctgtaa tcccagcact |
| 123301 | ttgagaggcc gaggtgggca gattgcttga gctcaggagt ttgagaccgg cttgggcaac |
| 123361 | atggcaaaaa cccatctcta caaaaaatac aaaaattacc caggcatggt ggcacatgcc |
| 123421 | tgtggtccca gctacttggg aggctgaggt gggaggattg cttgagccca ggaggctgag |
| 123481 | gctgcagtga gctgtgattg tgccactgca ctccagcatg ggtgacagag caagaacctg |
| 123541 | tctcaaaaaa aaaaaaaaaa agaaaaagaa aagaaaagaa attcactggc tgggcatggt |
| 123601 | ggctcatgcc tgtaatccca gcactttggg aggccaaagc aggaggatca cttgagccca |
| 123661 | ggagttcgag actagcctgg gtaacaaagc aagacccccg tttctacaaa aaatttaaaa |
| 123721 | attagccggg tgtggtggtg agtaactgtg gttccagcta ctggaaatgc tgaggtggga |
| 123781 | ggattgcctg agcctgggtg gtcaaggctg cagtgagccg tgatcatgct actgcactcc |
| 123841 | agcctgggca acacagcaag acttggtctc aaaaataaat aaataaaagt tcatccaatt |
| 123901 | ccaagatcag aaatacattt gatgagcaat aaaaggggaa tgaaactaca aattctaaca |
| 123961 | gagacctttc ataaatctga aagatagggt ggttgtttct gcctgggact tcatggagaa |
| 124021 | agagcaacat ttgaactgaa tctggaagtt ctgagttggc caggcagatg gcgatggggg |
| 124081 | tgggaagggc aaagccagcc acatatacac atgtggcatg aaggaatgag acagcttgat |
| 124141 | ggattctggg cacagttgtt tgggatggct gtgatacagg ccacaggtgg gaagtactga |
| 124201 | gagatgaacc tggaaaggta ggataggatc aggtccaagg tcctgaatgc caggctgaga |
| 124261 | atcccaagtt caatcccaaa ggccttcagc tcacaggagc caaggagtaa taaggtcaga |
| 124321 | tttgttggaa agattccagg gctggtgtga agactacact gtaggagacg gggaccagga |
| 124381 | gggcagctgg ggctatggta caagagacag atgagacccc ggctggttgg gagctgcagt |
| 124441 | gaggcaggta ggcatttgag atatctttta tcaggggccc tgcatccatc tcccatgtct |
| 124501 | tctgcaacag ccatctcccc tcataggtca gatcctgatg cggctggtgg aggagaaggc |
| 124561 | ccctgcttgt gtgctggaat tgggaaccta ctgtggatac tctaccctgc ttattgcccg |
| 124621 | agccctgccc cctgggggtc gccttcttac tgtggagcgg gacccacgca cggcagcagt |
| 124681 | ggctgaaaaa ctcatccgcc tggccggctt tgatgagcac atggtcagcc tcccatctcc |
| 124741 | ccaacccaga tttttgtcac cccaggcctt gcccccagac atcccttgtg aaggactccc |
| 124801 | atctaaggag aaggaagcac ctccactctg gggactgtga tgctggatgg tgtgtgagct |
| 124861 | cctgccctcc tgtcccaagt catctgcaca ttattttctg ccggatgacg aaagaaacag |
| 124921 | ctaagaggaa ggtctctgga acccagcaaa tttgggtcca gctcttactc tgcctcttgt |
| 124981 | tagctacgtg accttgagca aagcatgcat cctctgaacc ttagcttctt cagaatggaa |
| 125041 | atcacaatac tgatcctgac ttcttaggtt ctgaggtcag aggaaatgtg agaacactca |
| 125101 | tgggaagcta agccaggacc tggcatgaag taagccagat cctggtgggg tcttgactgg |
| 125161 | gagaacaatt ccccccaccc tcacctccag ctccccctat ccccacaggt ggagctcatc |
| 125221 | gtgggcagct cagaggacgt gatcccgtgc ctacgcaccc agtatcagct gagtcgggca |
| 125281 | gacctggtgc tcctggcaca ccggccacga tgttacctga gggacctgca gctgctggag |
| 125341 | gcccatgccc tactgccagc aggtgccacc gtgctggctg accatgtgct cttccctggt |
| 125401 | gcaccccgct tcttgcagta tgctaagagc tgtggccgct accgctgccg cctccaccac |
| 125461 | actggccttc cagacttccc tgccatcaag gatggaatag ctcagctcac ctatgctgga |
| 125521 | ccaggctgag gtccaggccc aggggtactt actgatgccc acccccaccc ccacccaagc |
| 125581 | agggacctca aaatcccctc cctttcctgt ttggggcctt gacacacgct gggctcaggg |
| 125641 | ctagggagtc tctcttccca cctctgacct ctttcagcct ctacactgac ctcaagtgtc |
| 125701 | aagttctatc aggctgcttg gtctcactag gccccctctt tccagagaga accatggact |
| 125761 | gacagcaaga agcctgagct cccgacccag ctctgtcact gatttgctga gtgactccaa |
| 125821 | gggaatcccc accttgctct gagatttaat cttctctctt aacacgaagg aagctggatg |
| 125881 | ggagagctcc aggggcctcc cagttctcgg cctcagaaag cctcccatcc tcagcccatg |
| 125941 | ccattctggg tgggatcaga ggaagtggca atgagttaga cgccctgcag gaatagctgg |
| 126001 | atgcaagctg ggccagagaa aatggcacag aaccctggac ccagggccag ggatgccctg |
| 126061 | gccttcccta actctggccc acctagccaa ttaggtgtgg ctgatgtccc ttgagtgccc |
| 126121 | tcttcctaaa gcccaaaaga agatgctgga ctcctctggg ccccaccaac aaatagggaa |
| 126181 | tagacatggg tggaaaatca ctcctttgtc tttattaaag aaacttagac cagacctggc |
| 126241 | aatcaagggg tgaggtactg gccaggaagg tggagtaggt ttcaggccct ggggatttca |
| 126301 | agtgcagact gatggcctgg gaggggccaa agagaccaga tcctggcagc agctgaggag |
| 126361 | gtgcccaagg gcactttcag gcactggggc catcagctgg ttctgtgggc aggggttggg |
| 126421 | ggttgggatg cagggtagtt tgggctggcc tggaatctcc ctgaggccac cctgccttgt |
| 126481 | ctacctagat catccactgg tcctgatcct gttcgttgcc ttccatgtcc acctggagag |
| 126541 | gaggctgggt gtgggtgggg aggggcctca gccagcctca gccccagatc ctgcccctgg |
| 126601 | ctggatccag ggtttctgta ccccttggcc atcaactggg tcaggagcaa gggtccagga |
| 126661 | acagaggccc tcccccatac cccttgccta cctcattgac ctctccatca tccggtgact |
| 126721 | cattgtagtc attcatctcg tccatgtcct gcatatcctc atcatcctct gagtcctctt |
| 126781 | cactatcctc atcatcttca tcatcctctt cttcctcgtc atcatagtgc tggtgggcag |
| 126841 | gacagagcct gtaagcccta caggcctgca tggaccagtt caagaactga cccacttgag |
| 126901 | cctctctcta gggccaatga atgaccccct accccgacac tccctccttg agtctagcag |
| 126961 | gctggtgcat gttctgcagg accttaatgc taggcccaat gcccacccct tctatctccc |
| 127021 | cttttaggct tttacccaga tctgagaacc acaactgctc tgggtcagag acaggacatt |
| 127081 | cagaattaga gcagagcctc ggtccactgc ggcccccaca caggccccac ctgctagagc |
| 127141 | cactcacctc tgaggctggc ttgccaatag gaaccaggtt gttgtctttc tccgcgatgc |
| 127201 | tttggagctg tgggcaaagg cacagaggaa caaggccaga gcccaagtag ggcaggtcag |
| 127261 | gggcatggga ctggcccatt ctgcccagaa gacaacccac acgtgttggg gagaagcttc |
| 127321 | ctcccagttc tcagggagat acaatccctt tcttgtcatc tgccatttat gaacttgatc |
| 127381 | caaatactta aactctctga gccttctttt ttttgtataa ttatggtgag gatgaagtga |
| 127441 | gagacttatc agcctagaaa gcatcatctg ctagatcctc aattaatagt caaaattatt |
| 127501 | tcctgtgctt tagggaaaag agcttgggct gagtgtcagc agcctgggct ctaatctggc |
| 127561 | ccagaagtca catggtaccc ctgtaaaata gggtaccaca agatgagaac cttggctccc |
| 127621 | tggctcccag cccgattatt cccagctcag ggcaaaaagc tcttcctggg tagatttctg |
| 127681 | gagtaaggct gggtcatggc ccactgaagg aggtctctgt ctgtgctgRg gtctctgtgc |
| 127741 | tgtgctagct gctcactcac ccaggcctga tgctgctgtt cctgctgctg cagctctgtc |
| 127801 | tcttccacac agggtcgatc cagattaaac cacagagtct cagtcacacg agggaagagt |
| 127861 | gaggggaaca aagtggacat ggctcctaga ctgagggaaa gggtcaagtg aatgtgtttt |
| 127921 | gctttgtttt tgtttttaat ttggttgtaa tttgatttRg ggatagggtg gaagacagca |
| 127981 | ggaaagcagc ccctccccct aggaatcaga cagacctggc tttgagtcct ggctccacca |
| 128041 | cttactagct gtttgacctt ggctgagtca cttaacctct ctgagcctca gtgtcttcat |
| 128101 | ctgtaaaatg gggataataa caattatacc actgtcaaag ttattctgat aattaagtaa |
| 128161 | ataatggatc aacaaagctg tttgcaggct gttcaaggag ggatgagaat gggagtcaag |
| 128221 | tgtagggata ggaatccaga atgcagactg gcattatgaa attatacaaa attagaggta |
| 128281 | gcagggttct actgaaacta tccctacctc ctccacacac taaaacctgc tttttgaccc |
| 128341 | tttatgatgg cccataccac acccacccag tgtcagaaat ttaggacact aacgacctct |
| 128401 | catgaatcac agccaaatgt cggggtggag ggattgtagc atatgcaagc agggttcagt |
| 128461 | aagacattct aggaggcatg aataagtgga taaataagta gatggaagat tgaatggatg |
| 128521 | aactaacatt ataagggtac aatcagcttt gagcggctga gggagccgac caggagtgcc |
| 128581 | agataatctg aattaaactt aggacaggcg aactgaggcc cagcgacagg cagagactgg |
| 128641 | ctttaggtca tacagtgtgt cggggcttag aattcctccg tgaaagggat Ragcaggaga |
| 128701 | aaataatcga tgcctggtcc tgcttttgtc ttaatttatc tgaccgaccc agggccagcc |
| 128761 | gctgcccttc cctgtatctc agattcccaa cccgcgtaat taggcacgac ggtaaacacc |
| 128821 | ctcatgtcca agaagtactg acagaccagc gatctcggct ccgaaagccc aacccaccgt |
| 128881 | cgcaaagcca ccgccctttc tttgctcatt acgataatga ccatgcagtt gaccaacaga |
| 128941 | aagaaagcag ggcgggtctc agacagaaat ttggccttat agggttgcta aacgcgaaaa |
| 129001 | aagcggggcc tgggtaggac caattaactg aaagaaatgc tgggtgacag gaagtgacac |
| 129061 | caatgaatga cggaccttgt gtgacSgggt ggggcttagt ctcgaggaag cggccaatga |
| 129121 | gctggagctc tttaagggtt aaggactctc aatatggagc tcctggggag tcgggtcaaa |
| 129181 | ggaaatgggt ttctctgggg gaaaaaggaa tgaaggggca aggagacggt tgcagccttg |
| 129241 | cgtctgtact taccgcgccg ggaggctgct gccggcggcg acccggaagc gcttcaccca |
| 129301 | gcctgagcgg aagaacccac cagaatccgg gactcaccaa acgcatgcgt ccttcgtctc |
| 129361 | cttttgtttt ccccgctccc gcccactaaa ccggatgtga cgttgaccta ccttagtcac |
| 129421 | attgttaggg aaggaagtgt gccgcgccta cctatctgcc ccSccttgag tctcagccag |
| 129481 | tcggcgtctc catcctggcg ccagcactac ggtctcgttt aaccactgcc ccgtttaacc |
| 129541 | cctgcccttt tgttgccagt caccatttgc ttgttaaccc cttctgaaga agagctgaca |
| 129601 | gcaacctttt agtgcgaggg ccattcgtcc tgctgcatcc cagaaaacta atctgttact |
| 129661 | acttcagaat tgctggttga tgttaggccc ctcctatctg tgctctctca gctacagttt |
| 129721 | cccgtttgag catattcatt cttttttatt tttgctctga acaaaaatat tagagttaca |
| 129781 | atattactat attccaggcc ttgctagaaa ctggggataa atctaggaat atggtcgctt |
| 129841 | ccctggaaga cctcacagtc cagggaagcc aaaccctgca gacatgcagt agacttagtR |
| 129901 | gtctctctta aggttgcttg ttgagttttg acattggaga ttatgtacag acttgaatga |
| 129961 | ctagttagcc tcaggcacag cattctgttg ctgggggaat cagctgccct cctcaggcct |
| 130021 | gggccaagta cacttagaga tcgccctcgt cacctctccc atcctttgct gatgcctctg |
| 130081 | ttctagtaac ctctgactca gcttcgcctt tagagatact catgctttct ggcaacagag |
| 130141 | gtccttcaaa cccaattcct attaaactcc atcacttacc agcctctttc agggacagca |
| 130201 | gttatccgca tttccagtac tgtcctggcc agatgtgtga gttttggcaa ttccttttcc |
| 130261 | tctctctgga ctcagcttct ttgtcaacca gcatcataat ctctgcccag ccttccttct |
| 130321 | atcctgggct gatgtgaaga ttggataaga attgttcaag aggtcgtgta ctgtgcatat |
| 130381 | tgaatgtgat tatattgcca aatagcactg tttgttatag tttttcatta aagagtaaac |
| 130441 | tttgcctctt ggaaatgact acctttttca ttcattcaac aaatacctac tgagtgcctt |
| 130501 | agtatatgtc catcactatt cttagtagtg ggtatacaat agtaaacaaa acagaccaaa |
| 130561 | aaagtccctc ttttcatgca gccttcattc tgagagaggt aaacaataaa gaagatagat |
| 130621 | aaataaaata tgtagttgta agattgcagt aaataagaga aaaaataaat ctgagaagtg |
| 130681 | ggaataaaat atttgcaggg gaggtgtttt atttcagact ggggggccaa agtcactggg |
| 130741 | aaggtgactt ttgagtaaag gtctgaagga gcagagagcc ttctgatatt tgtggaagag |
| 130801 | catttcagag agagagaata caagtgcaga gggcctagaa tggcttgggc atagtgtcct |
| 130861 | ctagggagag taataggtag gagatgagga gttggggagc cagtggaggg ttgtgagcac |
| 130921 | agggtccaca caatctgatt tacagtttaa ctgagttact ctggctgttc tccaaataga |
| 130981 | ctggggtagg ggagacaatg gcagaagcag ggagaccatt tgggagccta ttatgataat |
| 131041 | ctagaggtag tggatgagaa gtggttgaat tctgaattta tttagatggt agagactaca |
| 131101 | aggtttgcta acagatggac aatgccagaa taagaaagga gccacgattt ttgggctgaa |
| 131161 | cgactagaag attggtgttg ccactttttt gagttgggaa ggaatgggga ggtgatgagg |
| 131221 | ggtttggttt ttttttgaca gggagcttag gtttgagtgt gttgagtctg agatgcccat |
| 131281 | tagagtagag atgttggggc ggcaggaagg catatgagtc tgcaggtcag ggcagagatc |
| 131341 | tgggctggat agatcatttg ggagtcattg gcatgtagat ggtgtttaaa gtcatgagac |
| 131401 | tggatgagat caccaaggga gtgaatgtag ttggaaaata gtttcctcca atattcagaa |
| 131461 | tttgaggaaa ctaggaaaag cagcaaacca gatggagaag gaaggccaaa aagataagta |
| 131521 | gaaagctggg tgagttgatg gtatcttgga agccatgtaa agaaagggtg gtaggagaaa |
| 131581 | atggtttact gcagcctcat tctgtacaaa attgtgcttt tgtttatttc cccaacagac |
| 131641 | catgagctcc ttgagggcag ggactgaatg gttactatgt ccccagggcc cagcatgacc |
| 131701 | ttctcctgga ttcctcatct tccttctgtg acctgtgtct ccatcagttt ctcctccggc |
| 131761 | atctttttct tacaggattc ttacctcagg taccatttgc cccgtccttc ctgataattc |
| 131821 | cgcctgctgg aaattcccta gtaacagagt ttcttttcag ggtcatcagc caggctcagt |
| 131881 | aagtaatgat gactggttag ctggtgacat ttattgagta ccaactgagt gccagacacc |
| 131941 | tttctaggcc ctggggacag aggggaagca gaaataattc ctgccctaga gaaaaataga |
| 132001 | gtatagtgag tactaagaca attatagaca attatagagg agggtaataa atgttaaaat |
| 132061 | caatgtatgt agagaataca tgagaacaca gaggagggat tgatgaatgg gttctgaaca |
| 132121 | aactaagttt agacttttgt tccagaaaag gaaacatttg agtgggcctt gagggatgag |
| 132181 | taggagttaa gcccactggt aaataagtag gaaaagaggg tgcacaaaga ggcatatttc |
| 132241 | attcaagggc atgtttggag aacattgaga atctgggcag aactagatat gtggagtgga |
| 132301 | gaaagatgag aaagaatgat aggggaccca gatagggaag gtctttggtg gccctatatg |
| 132361 | gagtggatgg gatgggatct gggcatcttg ggtctcacct ttgtcttatt gccgacttgc |
| 132421 | tggggacccc aggtagatgt ctccccattc acaagcttcc gtgttctcta acataatatc |
| 132481 | taaccatgtc gccagctctg atgtgagtcc ctggaaggga ttcccctagc ttgatccttt |
| 132541 | ccattcttag atttttacat ttgccttatt tgtgatttat acaaaccagg gcctaggaat |
| 132601 | ggaaagcagg gtcaggatgg agatttctca acagaaatca ataaatccag agcccagagg |
| 132661 | ctgaagatgg aaaccatgat tctatgcaga gaattctcta ggatccagga agggccagag |
| 132721 | ttgagtggtg tgaattcagt gctagtgaaa gctgcttact cattccttcc ataaaccttg |
| 132781 | aggagcatgg ctgaccctgg taggggacag gggagaatgt aacagtcaca gtcactacct |
| 132841 | ttcatacata tggagggata gttggtctcc agagagtttt cccactcata gtctccactt |
| 132901 | atccttagcc cagccctggg gattctgcct tcatttagca gatgaggaac ctgaggctca |
| 132961 | ggaaagtaaa gcggcttgtc tgagttgaag ctacaactca agaatgtcaa aatcttgcat |
| 133021 | tgttccacaa atcttcctta tgggctgata tgtgcttgcc gatggggact cacagaggaa |
| 133081 | ttcgatttga gtcctagtcc tctgcaacta cccagtatgg tgtggatacc agactaacgc |
| 133141 | aagtcattag gcccttgagg ccagatctct gtgtgattca tctgtatttc cccggcacct |
| 133201 | tgcacagtgt ctagcacaga gtagccactc aacaaatgtt tatataagat gggttttttt |
| 133261 | ccttgggtct aatggaatta tagttcacgt gatgaatggt ataacaggga taggttctaa |
| 133321 | aaagagaaaa gtgggaatgt taaaaagtga agagcagtgt gggaggggag gtggagaagg |
| 133381 | ctcctcagag aagatgagac cttgcaagag gagttaggaa agaatattct agtcaagagc |
| 133441 | agacagcatg tgcagaagtg cagaggcctg aaagggtgtg gtgtgcccag gacatggcat |
| 133501 | tgaccgaggg cctgttgtgt ggccatcgtg gtgatgtgca ctgtgaaaga aacaggacca |
| 133561 | ggagacagta ccctgtgctc aaaggacaag gcacagcctt ggaggaggac agggacatgc |
| 133621 | tgagaaaggc ttccttgagc aggggagact tctgctgggt catgaaggat gcttcatagt |
| 133681 | tcgagtcaga gaggaggaaa gaggaaacct cccaggtgtg aggggcctcc ttaggcaggg |
| 133741 | gctgtggttg gaaggaataa ggagaatgcc caggcaggag agcatggtgt gtggagggca |
| 133801 | ggctggtcgg gtgggagggg ctgcagagct ccactgggtg ggctagctga gggagtgcac |
| 133861 | cttagcgtgg agtataaggc ttgttctgcc tcatggtcac tcaacacctc atttatttcc |
| 133921 | catcattgtg gcctccctgg acctgactct gagtttggga tggatagatg cgttggaagg |
| 133981 | gatctctgtg aattatcttc cagttcccta gaaagccagc cccaggagag gtgctccagc |
| 134041 | caggtcccta tctgcatggt ggttttgcgt atgtttgctt accacaaata tatggtaagg |
| 134101 | tttcctcact aaggtttaat gggctaaggt ttcctcactc tgactacagc aaaatagatg |
| 134161 | caaagtttga agagaggccc tctgcattgg aggcaaaggc tgaggagaca cagacctaca |
| 134221 | cacacacaca ccccccctca ctttcatgca gaggtggtag gcagaatgaa ggctccctgg |
| 134281 | agatacccat gtcctaatcc tatggtgtgt atcctacctg gcaaaaggga ctttgcagat |
| 134341 | gtgattaagg ttaaggactt tcacatgggg tgattatcca ggtgggccca atctatttac |
| 134401 | atgaatcctt aaaatcagat aaaactttcc cagctgtggt cagaaaacaa gacgtgacaa |
| 134461 | ggaagaagca tcagagagat tctacattgc tgactttgaa gatggaggaa agggccatga |
| 134521 | gccaaggaac acaggtggcc tctagaagct ggaaaaggca aggaaacgga ttctccccta |
| 134581 | gggcctccag aaagacctac tgtcacccta attttttttt aattttttaa tttttttgag |
| 134641 | acagtcttgc tctgtccccc aggctggagt gcaatagtgg gatctcggct cactgcaacc |
| 134701 | tctgcctccc aggttcaagc gattctcctg cctcagcctc ccgagtagtt gagattacag |
| 134761 | gcgcccacca ccacacccgg ctaatttttg tatttttagt agagacgggg ttttgccatg |
| 134821 | ttggtcaggc tggtctcgaa cccctgacct caggtgatcc gcctgcctca gcctcccaaa |
| 134881 | gtgctggtgt gagccaccgc acccggcccc tgacacccta aattgagacg catgttacac |
| 134941 | ttgtaacctg cagaactgta agttaataag cttgtgttgt ttgctgggca cagtggcaca |
| 135001 | ttcctgtagt cccagctact cgggaggctg tgataggtgt gttactggat agtgctctgt |
| 135061 | aatttcaggc tcttggtgtc ctgaacaaag aactggacca gacacacaca gatattaagc |
| 135121 | acagtattac actcttagag ggggagagtg gactgacctc tggcagatga gatcaacatt |
| 135181 | agttttgtgt actctgggtc tttgtgtgtg tgtgtgtgtg tgtgtgtgtg tgtgtgtgtg |
| 135241 | tgtgtgtgtg tgtgttctct tcccaaggct gcctaatctc tagccagtgt ctgccttttt |
| 135301 | gattgataga tgtgttgctt agttactttg gcccttgtgt gtcacctcca tcccataatt |
| 135361 | ttaagtacat gagtgatttg cagtccatat gcatgagctt caatgagcta attaccaaac |
| 135421 | ggggtcattt taaggatact ttttctcttt aatgtgcatg cccatctctg aggagctgcc |
| 135481 | cgcaacaggt ttggtccgga tttagcccag atgggggctt ctttttcact tttgttttgg |
| 135541 | ctgttctggt ttttatctcg cttcttgctc acctgcccct tcacctggct tctgctccct |
| 135601 | gcttttactc attctgccct tgatccaaat tttaattccc tttgctattc tcctgctgta |
| 135661 | ttttccctct tcccctgctt caggaggatc gcttgaaccc tgaagtttgg gaccagcctg |
| 135721 | ggcaacagag cgagaccttg tctcaaaaat caattaatta attaatttgt gttgttttaa |
| 135781 | gccacttagc ttgtggtagt ttgttatggc accaacagaa aacaaatgca caggtacaca |
| 135841 | cgtatatcct cccaccctga tagacaacca cagtcataca cgctcaggca gatacaggca |
| 135901 | gggagaccca ggaacatcag ctggccctaa gtgggggctg gggagaaatg aagcttcagg |
| 135961 | tgtgtgctgc ggggtgggtg gtaaggcctg ctccttggct ggtgtggaga aactttgacc |
| 136021 | acatttctca gtcttaactt tgctctgctt cctctcagtc cttacgtcat acttttagca |
| 136081 | acctttacaa caacattttg tcctaaaccc tgttgcaaat acatgtatga acctcaaaga |
| 136141 | ctatatatta aatgaaataa gccagtcgcg aaaggacaaa tactgtagga ttccactttt |
| 136201 | ctgaggttcc tggagaagtc aaattcagag acagaaaata ggatggtggt ttccaggggc |
| 136261 | tggggagtag ggagttatta ctgttttggt atttttttgt ttttacagat atcctgtttc |
| 136321 | tacttaaaga gttattaccg ttgtcaagct gggcatggtg gctcacgcct gtaatcccag |
| 136381 | cactttggga ggccgaagtg ggcagatcac gaggtcagga gatcgagacc acgctgaaac |
| 136441 | cccgtctgta ctaaaaatac aaaaaattag ccgggcgtgg tggcgggtgc ctgtagttcc |
| 136501 | atctactcgg gaggctgagg caggagaatg tcttgaaccc gggaggcgga gcttgcagtg |
| 136561 | atccgagatc gcccactgca ctccagcctg ggcgacagag ccagactccR tctcaaaaaa |
| 136621 | aaaaaaagag ttattaccat tgaccatgaa caacacaggt ttgaattgca tgggtccact |
| 136681 | aatatgtgga tttttttcaa tcacagataa aaaatacagt attcagaggt tgcgaaaccc |
| 136741 | acatatatga agggtgggac atcagtatgt gtggatttgg gtatgggggg ctcctggaac |
| 136801 | caatccccta catctgttga gggaggattg tatttaatgg atacagtttc agctggggaa |
| 136861 | gacaaaaagt gttctggatg agcctggtca tggtggctta tgcctgtaat cccaccattt |
| 136921 | tgggaggccg aggtgggagg attactcgag cacaggagct caagaccagc ttgggcaaca |
| 136981 | tggcaaaact ccatctctac caaaaataca aaaattagcc aggtatagtg gtgtgtgtct |
| 137041 | gtggtcccag ctacttggga ggctgaggtg ggaggattgt ttgagcccag gaggtagagg |
| 137101 | ttgcagtgag acaagatggt gccattgcac tccagcctgg gtgatagagc aagaccctgt |
| 137161 | ctaaaaaaaa aaaaaaaaaa agagttctgg atggattcac aacaatgtga atgtacttaa |
| 137221 | tgtgagtgta ctgaactgga catgtaaaaa ttgttaaaat gataattttt attatatgta |
| 137281 | tgttgtcaca attaaaaaaa aaaacaactt gtagattagg caccattttg gagaatgggt |
| 137341 | tcgggggagc ctccagtcga acttccccac tgcagtgcag ctggaaactc agtaactggt |
| 137401 | tctctgacac ctccccgcca ctcccacccc agtgccacat atccccatct ccatgcccca |
| 137461 | ctccagtcca gagccatcta tcgcctgggc tactgcaata actggctagc ttcccgccac |
| 137521 | accacttcaa accatacgtg gctgcctaaa acctttccct ggcttcccat tgcttttagc |
| 137581 | ctgtgagttg aagtccttgc caggcaagat ctgcccctgc cttcctgtcc accctcatct |
| 137641 | agagccaacc tcgtccttgc tttctgcacc ccaaccacac cccaacagtt ttgcagatct |
| 137701 | tcagacccac taaaacatgt tctgcctcag ggcctttgca caatgctccc attgcctggg |
| 137761 | actctgtcct tcttctttac tgggtcagct ccctccRttt tggtctcaag agaggtcccc |
| 137821 | cctgcagatt agctctggtg caaggagcac tcttccatag catcttctct tttcctcatg |
| 137881 | gcatctcacc taatacatac actcacacac acatttatgt gtgtgtgtgt atatgtgtgt |
| 137941 | gtatatatat aaaacatata ttttatatat aaaatatatg ttttatatat ataaaatata |
| 138001 | ttttatatat aaaaaatatt ttatatatat aaaacatatt tttatatata tatataaaat |
| 138061 | gtatggtaaa atatatgtaa caaaaaattt acaattttca cctttttttt tggacactga |
| 138121 | gtttcactct tgttgcccag gctggagtgc aatggcgcga tcttggctca ccacaacctc |
| 138181 | cacctcctgg gttcaagtga ttctcccacc tcagcttccc aagtagctgg gattactaca |
| 138241 | ggcacgtgcc accatggcct ggctaatttt gtatttttag tagagacggg gtttctccat |
| 138301 | gttggttggg ctggtctcaa actcccgacc tcaggtgatc tgcccacctc agcctcccaa |
| 138361 | agtgctggga ttacaggcat gagccactgc gcccggccaa ttttagccat ttttaaatgt |
| 138421 | acagttcagt ggcattaagt acattcacat tgtgtgtaac catcactaca tccattcccc |
| 138481 | agaactctca tctttcccag ctgaaactct gtaattaaac agtagctccc cacccagcct |
| 138541 | ccccaagccc ctggcaccca ccattctact tagtctctat gaatttttac tactccaggt |
| 138601 | accttaaatg gaatcataca gtatttttct ttttgtaatt ggcttatttc acttagcata |
| 138661 | atgtcttcaa agttcatcta tgaagctgtt gttgcatgtg tcagaatttt cttccttttt |
| 138721 | aaggctgggt aatagtcatt tgtacatatc acatgttgca tatccattca tcttttgata |
| 138781 | aacatttgga ttgtttccac ttttgggcta ttgcaaatag tactgctata aaaatgattg |
| 138841 | aacaaatacc acctatttta actatacatc taattgtgtg cttattcgtt taaattgtgc |
| 138901 | ctctcttcct catctctggg catcacaagt gttcaccagc atatcctcag cacccagcac |
| 138961 | agtacctggg catcagtatt caacaagtat ctgttgaatg aatgaatgag acaccaaaat |
| 139021 | atacctactg gacatataca gtgtctgtag tgtgtaagga agagcagagg acttgaatct |
| 139081 | ggggcccagc tcattgattc ctcattgcgt gctctgacct tgaccaaggc ccttcacctt |
| 139141 | tgtaaaccaa agagtatctg agacaaatct caaccaattt aggaagtttg ttttgccaag |
| 139201 | gctaagcatg cacccatgac acagcctcag ggcgtcctga caacatgtgc ccaaggtggt |
| 139261 | tggggcacag catggtttta tatattttaa ggagacatga gacatcaatc aatatatgta |
| 139321 | agatgtacat tggttcagtc tggaaaagtg gaacagcttg aagtgggggt gggggtcttc |
| 139381 | ctggtcatag gtagttaaga gataaccatt tgcattcttt tgagtttctg gttagccttt |
| 139441 | ccaaaggaag caatcagata tgatatgctt ttatctcagt gagcagagag atgactttga |
| 139501 | gttctgtcta ttctttgtcc acaaattatg aggaaggtgt gtagtttttt ttctttttaa |
| 139561 | tcttagtagc tatttttctt ggaatagaat gtgaggcagg tttgccctaa gcagttctca |
| 139621 | gcttgactat tccgttagta attgacttgg tcttagtaat tgacttttcc catagtaatt |
| 139681 | gacttgccct tagtaatttt ggggccctaa gatttatttt cctttcacac cttcaaacct |
| 139741 | cagtttcccc atgtgtaatg taggggcact atcagcccca cagagttctt gtcagggtca |
| 139801 | aacgaaacaa tacatgtgat aaggttcggc ttctgaccca aggaactgct cagtgaccta |
| 139861 | atctagaggc cttgatctaa gtactgaggg agagaggagg ctccaactgg gggagctgac |
| 139921 | tgactggatt cgaatccagc ccaaggtctg ttacccatcc tctggggacc ttggctctgt |
| 139981 | attcatttcc tgtagctgct ataacaaatt actacaaact tcttggctta aaccacacag |
| 140041 | atttgttatc tttcaattct gaaggtcaga agccataaat caatgtgtca gcaggactac |
| 140101 | attccttgtg gaagcttcca agaacaatca atttccttgc cttttccagc ttccagaagc |
| 140161 | cacctgcatg cctgggctgg tggcctccct tccttgtgtc cctctaacct cttgtttcca |
| 140221 | cctttatgtg tcctactact ctctctgagc tcctgcctcc ctcgcatcag gattacatca |
| 140281 | agtccattca gaaaatccag gatcatctcc ccatctcaag acccatttct taatcacatt |
| 140341 | tgcaaagtcc cttttgccat ataaagtagc attcagatgt tccagagatt aggacgtgga |
| 140401 | cacaggtacc atctctctcc catgtgggcc tcagtttgca ttttaggaaa ataagagcat |
| 140461 | tggtcatgcc ctgtcctccc tgtcaccctt gctctgtgga gttctacctg aggcaggaat |
| 140521 | attgggtgga ggccagaggg ttgggtcctg cctgcctgac taccctagca cccaaattct |
| 140581 | cacaatcttg tcaccagcag ttatagcccc agggcctggc tgtcccccaa cccgtgttcc |
| 140641 | agtgccccac tgttcccttc catttcctgg gtgcttctga ttcaggactt tttcctggac |
| 140701 | ccttcactgg actcacaaga ggggtgccct gtttattcag tccaccacgc tcaatctctt |
| 140761 | gcaggaggga gcacatgagt gagtgagtgc aggatctggt tggctgatcc aggtgctggc |
| 140821 | agcagcaagc tccatgcagg gccctcggcc agtctagatg tgagcgagca agggcaggat |
| 140881 | ccagccggct gctctgggca ctggcaggag aaatctccat gtggggcctg cggcagtgcc |
| 140941 | caggtggggg tgcccatgac ccctaagccc cagagggagt attacagtgc tctcctagtt |
| 141001 | ctgctgtcca tggacggtgg tgtgttagca gctcagttgg ccccttgcat cgtctcatgg |
| 141061 | ggtggttgcc ctctgccagc aagggcaaag agccagtgtg acagcctttc tgggtaccca |
| 141121 | cacttggtgg gtcccgagcc cttggccagt gtccaagaag aatgaggttg tgcagacaat |
| 141181 | tgaagaatga tgaaggtgga gaattttatt aagcaatgaa aacagctctc agcagaaagg |
| 141241 | ggagctggag aggaaacagg aagggccagt catcttcccc cgaagtcagg ccatctcttc |
| 141301 | tgtagtccag ccatctcttc ctcaaagtcc ggttgtctcc tcgaagtcca gccatcttcc |
| 141361 | catctactga ctgagtctgg ggcctttata tgcacaggac ggggagtgca tgctgattgg |
| 141421 | tttgtgagta tgcaaaacag gttaaagtga agacatcact caaagatggg catgacagtg |
| 141481 | tagaaaacca attaggaaag ggtaagtata tgtaaaatag gtgaagggtg gggatcaatc |
| 141541 | agaggaaagc acaacaaatg ggaagacaag tcttcaatct ggtctgagga tttagcttgt |
| 141601 | agcttggctt tcaggcttta aactggcttt ggcttggaag tggggtttca ccagggacca |
| 141661 | gtccctattt gcctaggcat ttggctgcct cctgtccctc tcatttcccc ctctgaagag |
| 141721 | gtacaactac ctgttgttag aatagggaca aagacaatct taactacttc ctgctgacag |
| 141781 | gggatgctgt tttgagcaaa cagcaatcag agctccctca gaggcctatc taagcgtccc |
| 141841 | aggttaaagg gagccatcat tcaaggctct gttcggaatt tgatggcctc taagtgagaa |
| 141901 | gaaacagttt gggttattag atgacatgga tcaaaatgga acaacaagga ggtaaagaca |
| 141961 | gctcaaaaat cccaagtctg ctgacatgcc cagataacta gtggctatag ttatgcctgc |
| 142021 | taagatttgg atgcatggtg cttggctttg gtttgctccc ttgttttcat tttcccaaaa |
| 142081 | aaacctccag attatgagca ccacatccta tttactccta tcagctgcag gataattgcc |
| 142141 | cagaactaga atattgattc atatttttac attacccatc ccctctattt cttctgagct |
| 142201 | acaaccagag atcactagtt ggttcacagg aataagcagg attaatctaa aatgtaggca |
| 142261 | aaagcttaaa aacaactgaa actagaattt aatgacaagt gtatgataaa ttttgaaaca |
| 142321 | taatttttct ctctccagtc ctcatttctg ttaaaaataa accatgatag gactgagttg |
| 142381 | tttgcaaaat aaactttagt cttatacttg gcctggtcat ttgcataaag tgcagcaaga |
| 142441 | ataatcattt tcacataggc ttttaaaatt ggctttgatg gaactctgtt tcacaaggaa |
| 142501 | tctcaggtag gaccttttaa agctgagctc agccatgggt ttgtaccctc aaatacctat |
| 142561 | gagttgggta aattcttctt ttcttgaggt cccagataac acagggctct tgggcctgtt |
| 142621 | agaaaatgac attctctact caccacaggt taggaaccct gtccagggac tgtgtagaca |
| 142681 | aggtatgagg ccagttttcc caaggggatt ttattggctc tgcaagtcaa gcttgattcc |
| 142741 | ctaaaggaaa gcataccatt ccagtcaaaa ccttggtaaa ataaccggtt tctccagtta |
| 142801 | tatcctgctg caaaagaaaa tagattctta ttgcactgat gcaaataact atattgccat |
| 142861 | aagttaagaa tatttgcaaa tagtttccaa attctagaga aaccaggcag agagaaatat |
| 142921 | gctccaaatt ttcttcacag gagtatacct tactcaattg ttaaaagctg tagatagctc |
| 142981 | aaaagaagtt tccttgactc tggaaaaaac aaaacaagga tcagcaatgt tttaagcaaa |
| 143041 | aagttaaaaa ggattacttc agttttctat tagttcagtt tattcagtta actcttgttc |
| 143101 | tgcttgatat tcaggagaat ttcagctctt tatgactcct gtatgttttt cctctgttcc |
| 143161 | aatgtcacaa tctccaaagt catcagaaac ctgcatttga gggcaccttt caaagtccaa |
| 143221 | tagctgtcca caaatcatct tttgaaaagg gtcaaaacaa gacaacaatt gtctgtgaat |
| 143281 | gacaaaatat ccttgggtag tcacagtcaa aaacacaatt gacaaagaaa tttgtttttt |
| 143341 | ctctgtggtt tacaataact taacacaata accttaatta ttactgatag catatactca |
| 143401 | gacatttgag ttttagaagg cccatacaat taggccgggc accatggctc atgcctgtaa |
| 143461 | tcccagcact ttgggaggcc gaggtgggtg gatcatgagg tcaagagatc tagaccatcc |
| 143521 | tggccaacat agtgaaaccc tgtctctact aaagtacaaa aattagctgg gcatggtggc |
| 143581 | acatgcctgt agtcccagct actcgggagg ctgagggagg agaatcgctt gaaccctgga |
| 143641 | ggcagaggtt gcagtgagcc aagattgtgc cactgcactc tagcctggta acagcgcaag |
| 143701 | actctttctc aaataaatcc catacaattt tggaacatat attaatatta ttcactaaaa |
| 143761 | tataccctga agaaagtcat acattatttt tattttggca atcccatgta actaaatgtg |
| 143821 | ttaaataatc ctgtttacct ctcttttgga tgctccaggg gccttcagta gcatccaaaa |
| 143881 | gttaggggtt agaaaagaca accttgaacc tgaagtttga atttgggaag tctatcaaac |
| 143941 | acattaaaga tttaaaacac ttattatttt gaaatagaac tccagatcac cataagttat |
| 144001 | ttattttagc caaaatgatg actcaaaaat tttaaaacaa ggcaaaaacc tttcattatc |
| 144061 | ctttattatt acatgaaaat cttcttcgag agaaagtcaa atttcaccct tgcatgtaaa |
| 144121 | tccattttca gtagtctcaa ttacatgttg taatggtaac tcttagcaat ttttaatttt |
| 144181 | aatgtaaaac ctggtaagtt tttttaatta tgtactaggc acagataaag tctgactctt |
| 144241 | tacaacatag ttaatggtgt ggttaatcca tatgacccca gaccttacca aattgtaaag |
| 144301 | ctggcaagtc aaacagttct caaaagccaa agaagcagtt tataacctta caacatttag |
| 144361 | caaacctagt atctgacctg catacattag accacatatt ttcattttga taacatttgt |
| 144421 | attttaccaa ttatcttcaa aattgttttt ctttctcaaa gattaaagtc acatgaacta |
| 144481 | aaaggcaata cagcttttgt ttttccttca aaaaatattt gatctaagca cttattttct |
| 144541 | ttaagcaaat tagagctctt tttttatata aacatcacac acaaataaca catatgtgat |
| 144601 | tacacagaca gaagattcag tagttgtaag atttttcatt tgccaatctc ctaattggat |
| 144661 | tattggcctc agggtggagc atttcaagaa gcagagctag gaaagcatgc agtttccagg |
| 144721 | gcctaataaa caggcatagc tggaagacaa aaacagattt tgaaaggggt ctatctgctt |
| 144781 | ttaattcctg gggttctata agaaaaacag agtttttttt tttttttcca aaatgggatc |
| 144841 | cgtggtgact tctctgtttt tcccaaggtg tcttatgctc tcagaagtta tcttagggcc |
| 144901 | tctcatgcat gcgttaagag tgacaagaca aattggggaa aaataattca gttgactgag |
| 144961 | aaaaaattgt tttgcagaga aacaagatcc atgaagaaaa aaacacaaag gccttttaaa |
| 145021 | tatacctata gcttggatat ccacttttaa ttaagctgag cactctttaa gataaccctt |
| 145081 | ttaaatccct tagtacccaa ctttagccat gccaagccac gaatatttct ggcttttgaa |
| 145141 | ctttaccatt tctttcaaca ttttgcacag ggagagagag gccagaggcc cgactggcag |
| 145201 | aaaactttta cccttctgtc cacatgttca caattctttc cccttctcac tccatcctta |
| 145261 | ttccactctc agccttcctc atctcagtaa atagccaagc actcctctct aattgctcag |
| 145321 | accaacacac tctggagtca tcctaattcc tccctttctc tcatgtcctc catctcagca |
| 145381 | aatcctatca aaacatcctt caaaagacat gacattactc ctcccttttc accacgtcca |
| 145441 | ctgccaccac atagccctgg ttcaagtcat ggttgcctta aggctgggcc attcacatag |
| 145501 | acatctgtgt caattgtttg ctttcccttg tccctagagt cagatcagat gctgaactgg |
| 145561 | aaaaagcaaa taaagtttcc atttcattca gagtaaaatc ccccatcttt gctgagtctg |
| 145621 | aaccacgggg ccctgaagat ctggcctgga ccccacacct gccgctccca gctcactgct |
| 145681 | ggccacactg gcctcctcct cgctgtttgt tcctgaaatg cactgagtac atttttgctt |
| 145741 | cagggccttt ggacctacgg gtccctctgt ctggagcact ccttcctcca tgttcacatg |
| 145801 | actcccttct tacctcatta tggtatcagc ccaagcatca cctcctagat ctttcctgta |
| 145861 | gaagatagta gcccctgctt catcactctc tgtcccctta gcctgcttca gttttcttca |
| 145921 | gagcacaatc atcatcttta tttgttttct gtcgtctctc tctaacaacc tcccaccccc |
| 145981 | aaactagaat gtcagctcta tgacagtagg gactttttct tggtcacttc tgtagctcta |
| 146041 | gaacccagaa cagcctagca catggtagat gcccaataac taattctgag cttagttttt |
| 146101 | gaatgaatcc ccacttctac cctcctggtc aaggcaccat catcccaggc ctggacttgt |
| 146161 | gaaacagctt tctaactggt ctaccttgcc caattccagc ctacacgtaa tcccatctga |
| 146221 | acacaccaag actgccaatt gaactgtgac attcccctgc ttaaaactct ttcactgtga |
| 146281 | ccagttgtag aaacaacatt ttgtttatcc atttatccat ccgtggacac ttgggttgct |
| 146341 | ttcaactttt agctatcgtg aattaatgct gctgtgaata tgactgcaca gatagctgtt |
| 146401 | tgcatccctg ctttcaattc ttttgggtgt gtgcgcacaa gaggaattgc tggatcatat |
| 146461 | ggtaattcta tgtttaattt tttttcacag catctggacc attttacatt ccccctccac |
| 146521 | tagatatgtt attatataca aaggtggacg gctatacaag gacattccct acagcattga |
| 146581 | ctataacagt gaaatatgag aagccactta aattcaatag aataatgaaa tagaatcata |
| 146641 | tagatctatg tacacagata agggaccatc tatagtacat tctgttaagt aaagcaagca |
| 146701 | aagtgagctg ggcacagtgg ctcatgcctg taattctagc actttgggag gctgaggtgg |
| 146761 | gaggatcact tgagcccaag acgtcaaggc tgctatgacc tatgattatg ccactgcact |
| 146821 | ccagcctggg tgacagagag agaccctgtc aaaaagaaaa agaaaaaaga aaagaaatca |
| 146881 | aaatgcaagg gaattagggg ttattcttct tcctgaggac tggtgccatg gcatccagag |
| 146941 | aaagaacatt ggtggggata ctgatgaaaa agaataaagt ttgtagatta gttaaaagta |
| 147001 | ttcccttaat gttaatttcc tgatttttta agtaattggg ctttggtttt gtcagatgcc |
| 147061 | aacatttggg gaaactggat gaaagcatat gggaaacctc tttactactt ttgcaatttt |
| 147121 | tctgttaagt ctaaaattat ctcaaaacac taagttaaga aaatcaaata aacttgatgt |
| 147181 | cttaaaacaa taccaattag gctgggcgca gtggctcatg cctgtaatcc tagcaatttg |
| 147241 | ggaggctgaa gtgggccgat aacctgaggt cgggagttcg agaccagcct ggccaacatg |
| 147301 | gtgaatcccc acctctactg aaactacaga aattagcctg gcatagtggc atgtgcctat |
| 147361 | aatcccagct acctgggagg atgaggcagg agaatcactg gaaccggaag gcagaggctg |
| 147421 | cagtgagctg agatcacacc actgcactcc agcctggggg acagagcaag actctgtctc |
| 147481 | aaaacaaaac aaaacaaaaa cacaatactt atttacttat acttcaagat tttgtgagtt |
| 147541 | taattatact tcaagatttt gtgagatttt gcccaagaat ttgggcaaga ctcatgattg |
| 147601 | gcccatcttg gccgggcgtg gtggctcacc cctgtagtcc cagtattttg ggaggcctag |
| 147661 | gttggaggat tgtttgagcc caagagttga agaccagcct gggcaacatg gcgaaacctc |
| 147721 | ctctctacaa aaatacaaaa attagctggg cgtggtggtg cgtgcctgta gtcccagata |
| 147781 | ctcagggagg ttgaggctgc agtgagccat gatggtgcca ctgcactcca gctggggtga |
| 147841 | cagagtgaga ccctgtctcc taaaaataaa aaataaggta aaataaagat tggcccatct |
| 147901 | accctcaaca gttgcctgga ctccctgggg ctcagtgcac ctagatgctg gactcggata |
| 147961 | accatctgat ctttgatgta gtggacagac actttgttct tgccatcatc tttggtacct |
| 148021 | gagatagaaa gccttaaaga gaggccacgc ccatgattaa gtttcaggtc aaaacaacga |
| 148081 | gcaaaaaggc aacggctata gttgtaggtg tagcaaatca gtgtgccttt tatctagagg |
| 148141 | ctactgaaaa ccagacattc attaaagata ttctggtcac ttggatgaag gctggccttg |
| 148201 | gcattcacct gaatctttag tagagtcttt agcaccattg gcaactcagc cagacctggc |
| 148261 | cttggccacg atagtggagc ctgggattgg agggcatgaa ttttatgaag cctgtaatat |
| 148321 | cagaaattcg ttggttgagg acatacctct ttctttttgg accctgtggg cgtaatgcta |
| 148381 | ttgatacatg tgcagaggag gaattaatat gactgtgatt gacagaataa ttatgaagtg |
| 148441 | caaagacctc agatctgtga tcagcttcca tagaagtatt tgctcagaag cttctcagaa |
| 148501 | acattctcat gattgaagaa gccttgaaga gcccgtattt ctgtctatga aatctcctgt |
| 148561 | ttattgtact atgaggaccc atcagaaagt aactgaggta gtgctgattt cctgagcaat |
| 148621 | tattcagtcc actgtctcaa atccatcatg cagaatgttc acagagggta aagtaagttt |
| 148681 | gactattata actactcact tttataagtg atttaattta aagattaata tgataagtac |
| 148741 | tctgtttttc ctgggaattt tgttgtagaa agagtaactg ctgtgtggct aaactaaggt |
| 148801 | tataagccaa attgtttcct cagagtttaa gaaacacaat actaactctc atgggtttac |
| 148861 | tagttgtctg ttgctgcata acaaatcatt ccataatttt gtggtgtatt gctgcagaca |
| 148921 | atgttaaact aagtggatga aaaggatatt cacatagtct cagagtgtct ccctacaagg |
| 148981 | taggattact aacaaaggga aactaataat tatatagtaa ggaaatctcc ttaacccaat |
| 149041 | aatcaccagc aataagatgc agcaaccctc atcatgtacc tcttgatatg atgcactgac |
| 149101 | aaaagcacct ctcttctcta attttcttgc caaaatgcat aagctcaagc taattacagg |
| 149161 | aaaatataga caaacccaaa ttgagggaca ttctgcaaaa taactgaaca gtaattctcc |
| 149221 | aaaagtgtca aggtcataaa agacaaagac attgaggaac tgtcacagat tggagggaga |
| 149281 | ctaaggggac atgacaacta catgcaacct ggaatcatgg actgaatcct gggccagaga |
| 149341 | aaggacattg ggggggaaac tggtgtaaag ggcataaagc ttgtagatta gttaacagta |
| 149401 | ttgcctcaat attaatttcc tgattttttt aagaactggg ctttggttac ataagatgcc |
| 149461 | aatatttggg gaagttgcat aaaaacatac gggaaatctt ttgacgatgt tttgcagttt |
| 149521 | ttctgcaaat ctaaaattat ttcaaaacaa aaagtttaaa aatcaaatac acatagttgc |
| 149581 | ttgaaatagt aactatttta ttatattcca agatgttgtg agtcaggaat ttggccaaaa |
| 149641 | ctcaggtggg cgattcttct gcaaagaccc ccacaacaca ttcaaagtca caggcagagg |
| 149701 | ttgttggggg agggcattga aaagaagaga agagtcatag gtgggtgcaa tggagggagg |
| 149761 | gcagagggct gctgactatg gtgcaggact catccataat ggagccctgg ggaggcaagg |
| 149821 | gcttcataac tagacactgg tcttgtcacc tcagactcac ctgtagcagg accagatact |
| 149881 | gaggtcagac tgaaaacaca ggctctgcct caggagaggc tctctactag ctgagtaaat |
| 149941 | gatgacagta ttggaaatgt tcccaacatc ataatgggaa aacatcactt cacactacat |
| 150001 | aagcaataca caggggcagt gccggtcgtc ttcccaggtt agtagcagtt ctactgcctc |
| 150061 | caagagtgtt ggagaaatac aaaccaagca ttaggcactt ttaacttgaa aacatgaagt |
| 150121 | tctctttcct aactttcttt gtttccttat ttcttcttct tcttcttctc cttcttcttc |
| 150181 | ttcttcttct tcttcttctt cttcttcttc ttcttcttcc tcttcttctt cttcttcttc |
| 150241 | tttcttcttc ttctccttct ccttttcctt cttctttttt tgctgagaca gggtctcact |
| 150301 | ctgacagtac agtggtgcca tcacagctca ctgcagcctc gacctccagg gctcaagcaa |
| 150361 | tcctcccagc tcaccctccc aaatggctga aactacaagc tcgcaccacc atacgtggct |
| 150421 | aatttttcta tttttgtgtg cagatgaagt tttcctatgt tgcccaagtg gtctcaaact |
| 150481 | cctgggatca agtgatccat ccacctcaac ctcccaaaac gctgggatta caggtgtaag |
| 150541 | ccaccacacc cagcccacta acttttttat atcggctaat gaaatagttt taagtttaga |
| 150601 | ccctacgagg cataaagaaa taattttagt tatgttatca gatgtacagt aatactcaag |
| 150661 | tgtgcaactg tggataactt gagttcatga ggtttttgtt tttttgtcaa aagaataaat |
| 150721 | ttatagtgaa actacccaaa aaagcaaagt acagaacagt atgctaccat ttgtgcacag |
| 150781 | aaatgggata tatatggtgt aactgcatcg aatttactgg atgtatgtcc agggaccaga |
| 150841 | actcttggtg gcttcatgtt catacttttg caagcacatg tgtagtatcc ttaacttaaa |
| 150901 | ggtactgttg tatacattct agtgttatca aaatttacat acatattatc aagtcagaga |
| 150961 | ggtcattctg tgtcttagta ttttcacttc atatttggta tatttatgta tgtatacaca |
| 151021 | catacctata tgtatttaaa taagatttat agtcacatgg tccaaaaatc aaaacaatgt |
| 151081 | ggaaaggttt acagagaaaa gtctcaagcc taatcctgtt ctctactgcc aggtgaccat |
| 151141 | gttattaatt tcttttcata ccttgccaca gaattttcac ctgcaaacac agatattctt |
| 151201 | ttctttttta atgacagagt cacgttctgt tatccaggct ggagtgcagt ggcgtgatct |
| 151261 | tggctcactg caaactcctc ccgggttcaa gtgattctcc tgtctcagcc tcctgagtag |
| 151321 | ctgggattac aggcatgtgc caccacaccc agctaatttt tgtattttta gtacagatgg |
| 151381 | ggttttatca tattgaccag gctgatgtcg aactcctgac ctcaagtgat ccgcctgcct |
| 151441 | cggcctccca cagtgctggg attacaggcg tgagccacca cgcccagtca acacagacat |
| 151501 | tcttactcct tttttacaga gaatttatta ttattatttt ttacatagca tttttctgca |
| 151561 | cctttctttt tccacttaac aatgcacttg aagatttttc catatttgta catcaggagc |
| 151621 | tttctctttc tttgttacca cattaaattc cactgggtag atgtaccata atttaactgg |
| 151681 | gtccttattg aaagacaatt gagctgtctc ctagacaaag ccttgtgcac cttcccKaac |
| 151741 | agagggtcta accaagcagg caggatgggg ttataaagta ggtggggagg tgggagagac |
| 151801 | tccaccttcc caggtgggct gagaatggag gtaaggccct gcaacaggac agagggaaaa |
| 151861 | gtggggatga gaggtgggag gcgagatagc gcccactgtt ctcgctcagc cccctcctcc |
| 151921 | gtttgccgct gacctgttgg cctcccccaa cctctgagcc tgcctctgcc taggtaattt |
| 151981 | cccaagaccc agaaggggtg aagggtgagg tgtgattgcc cccacctcct tgcctcccgc |
| 152041 | agcatctgct ccgggaccat gaacaatagc tgacagctcc atggcccttg ctgtccccat |
| 152101 | ctcagcttcc ctgggcatct aaacctcagY tgccatgggg taggaggaca ggctgaggaa |
| 152161 | gcagaagcct gaggctgtct agagtctcac tcctgcatca gcaggccacc acctgtggtt |
| 152221 | cctccttgtg caaatttgaa aagaattgca taaaacactg gagaaatcca agaggggaag |
| 152281 | tccacaaggg cggtggctcc ctacaaggtc acagagcaag ctggtgtcag aRcctggacc |
| 152341 | tacagcgctg ttggtggagg tcctgcctcc aggtagggga agggctccct ctcacctcta |
| 152401 | cacgcagcgc atttcttggc tcagctgccc tgtaggggat gcagggtggg gacagcagag |
| 152461 | atctgggcct gggagggaga gagtacacaa tcacatggct gttgcccctg tctcaggcct |
| 152521 | tgtctacctc tgactgtggc tctctggcag gaatagatgg acatggcctg gcagatgatg |
| 152581 | cagctgctgc ttctggcttt ggtgactgct gcggggagtg cccagcccag gagtgcgcgg |
| 152641 | gccaggacgg acctgctcaa tgtctgcatg aacgccaagc accacaagac acagcccagc |
| 152701 | cccgaggacg agctgtatgg ccaggtgagg gcagcctggt gtaggacagc atgcacacag |
| 152761 | gtcagagggt gatggcacga gcaatggcag gtccagtgtg gtcagaacca agggtgccgc |
| 152821 | tgctgacaag gaaggggagg ggcggccagg gccaccatgc cacaggtaag gccactgagg |
| 152881 | cagcttgggg aatatgagct ccaatttgaa ctccaggctc aggagtgtgc ttgtatttca |
| 152941 | ttcctctggt ctcctggcct gctccctaca aggtttcaca ttcccagagg gctggggatg |
| 153001 | tgcctaggga gagactgtgg cgtggacaca atctgtgggt taaagcgaag acaggacagc |
| 153061 | ctggaagccc catgacatct gagtcactcc caacattcca tttgcttatt tttaaatcgg |
| 153121 | ggttaaaaaa aaaaaacaaa tacataacat acattttcca ctttagccat ttttaactgt |
| 153181 | acggttcagt ggcattaggt atgctcatgt ggttgtgcaa ccatcaccac catccatctc |
| 153241 | ctgacctctt tcattctcca aaactgaaat ggaaactctg tgcccaccac ttcatttgct |
| 153301 | tttcagaacc ttctagagca catcctcctt gccaggaaat ggtgtggatg tagacctttg |
| 153361 | agagagacag atgactatca ttctcagggc catgagctat atgagagtga tgatatttgt |
| 153421 | tgagccctta ctatagcaag ggagttcttc tcattgtact cagtaactct tttggaggca |
| 153481 | acaacccttg accctgacag gcaggaccca tgtctgccaa accctaagac ccatgatgtg |
| 153541 | caaggggtct tgcaggaaga ccaagagttg gaacatccaa ggaaaagcaa gtgtgaagtc |
| 153601 | gggctggcag ggaagcatgt tctgtgtcag ccggcactgg gcgtgggcca gggtgtggga |
| 153661 | ggtgggtagg tctggctccc ctcccatgga tttccctatt gtttctcctg ggtgctcagg |
| 153721 | cctgtcacgc ctctgccatc acttgaccct aggtgcaagg gttcagccca gaaattttat |
| 153781 | gcaattgaYt catgatttct caggttttct gaRtcctggc ctagagtgac ttcccaagaa |
| 153841 | aaaMctccac catttctgct tgtcttacct gccttgtatt tacctttcta ggattgcctt |
| 153901 | ttccacattt agtcaagtct aggttcagac ccacgtgcag gctatagctc cttcgttctc |
| 153961 | caccactctc aggatctatc tagagtctcc ccacctggac ctccagaccc tgggagagcc |
| 154021 | agaccagccc cttgacctcc accctccccc acaacctggg ccaggttcct ctcctccctg |
| 154081 | tcctcagtta taattttttt tttttttaat ttgaggcaga gtttcgctct tgttgcccag |
| 154141 | gctggaatgc aatggcatga tcttggctca ctgcaacctc tgcctcctgg gttcaagtga |
| 154201 | ttctcctgcc tctgcctcct gagtagctgg gattacaggc gcctaccact gtgcctggct |
| 154261 | aattttttgg tatttttagt agagacaggg tttctctgtg ttggtcaggc tggtctcgaa |
| 154321 | cttctaacct caggtgatcc gcccgcctcc ttaaatctta acctcactgt ttaccatggg |
| 154381 | tgtagcttac ttaaactctg taaaaatggg ggtaaggatt cgtactgggt tgttgagagg |
| 154441 | ataaagcgca aaagcctcag ggactttgca cctatggttt tctatgccta gagtgttctt |
| 154501 | tgtctccctt ctacacacag cccacccacc cactaaaacc ataccctccc tgagggtaga |
| 154561 | ggtgttattt gttttcttca ctgtggtgtt cctaggacct agcacagtgc ctgatgtata |
| 154621 | atcagcactc agttaatatt ggctggatgc aaaatgaata atataaataa gctgaataac |
| 154681 | atgaaatagg ccgaacgcgg tgactcacgc ctgtaatccc aacactttgg gaggccaagg |
| 154741 | agggtggatc acctgaggtc aggagttcga gaccagcctg gcaaacatgg tgaacccccg |
| 154801 | tctctactaa aaatacaaaa ttagctgggc atggtggcac gtgcctgtaa tcccagctac |
| 154861 | ttgggaggct gaggcaggag aattgcttga acccgggagg tggaggttgc agtgagccaa |
| 154921 | gatcacgcca ctgcactcca gtctgggcaa caggagcgaa actctgtctc aaaaaaaagt |
| 154981 | tagtttaata acatgagatc actttttaaa ccgttaagag ctgtactact aataattact |
| 155041 | ctctcagaca gtggctctcc ctcatctcct atatcccgtg gattaccctc tgttaaaagc |
| 155101 | caaaattaag caggcatggt ggctcacgcc tctaatctta gtattttggg aagctgacat |
| 155161 | gagccaagga gtttgagacc agcctaggca acatagtgag accccatctc tacaaaaata |
| 155221 | ctttatatta gccaggcatg gtggcatgtg cctgaattcc agctcctcgg gaggctgagg |
| 155281 | tgggaggatt atttgagccc aggatgttga ggctgcagtg agctatgatc acaccactgc |
| 155341 | gctccagcct ggtcaacaga gcaagaccct gtctcaaata aataaatgaa taaatagggc |
| 155401 | ggaaagcacc aatattgtaa ttgcctccgt ccccaggtgg gagctcctca agggccctcc |
| 155461 | ccaggaagtg ttcctctgga tgacctacct ggggcagagg agccagaata tggaggagat |
| 155521 | ggctgtggtg gggagagact tagtcctgtg tcttccccac ccagtgcagt ccctggaaga |
| 155581 | agaatgcctg ctgcacggcc agcaccagcc aggagctgca caaggacacc tcccgcctgt |
| 155641 | acaactttaa ctgggatcac tgtggtaaga tggaacccac ctgcaagcgc cactttatcc |
| 155701 | aggacagctg tctctgagtg ctcacccaac ctggggccct ggatccggca ggtatgagtg |
| 155761 | ctgttcccac aaacattaac ctcagcagag ggcggagcct gccagttgct ggcagggagg |
| 155821 | gcttggtcca ggaattcggg tctgagggtg gtggacgccc tgccccctcc cacagctctg |
| 155881 | gtccccttca agggtaaagc tgctgagata cgtggctgac aggagtattc tgtctcctcc |
| 155941 | ccactcaggt caaccagagc tggcgcaaag agcgcattct gaacgtgccc ctgtgcaaag |
| 156001 | aggactgtga gcgctggtgg gaggactgtc gcacctccta cacctgcaaa agcaactggc |
| 156061 | acaaaggctg gaattggacc tcaggtgagg acctgaggag ataagatgag gagtgggagt |
| 156121 | ggggctttgg ggttgggagg ggtgcggtct ggcccagaag ctaagggtct tacgttctcc |
| 156181 | tccctcaggg attaatgagt gtccggccgg ggccctctgc agcacctttg agtcctactt |
| 156241 | ccccactcca gccgcccttt gtgaaggcct ctggagccac tccttcaagg tcagcaacta |
| 156301 | tagtcgaggg agcggccgct gcatccagat gtggtttgac tcagcccagg gcaaccccaa |
| 156361 | tgaggaggtg gccaagttct atgctgcggc catgaatgct ggggccccgt ctcgtgggat |
| 156421 | tattgattcc tgatccaaga agggtcctct ggggttcttc caacaaccta ttctaataga |
| 156481 | caaatccaca tgtgtcttgt gtcttgtaat ttcgggacga gtgggttgga gggacacatt |
| 156541 | gcttcatctt ttccattgac aggccccaaa ttgggctgga actagcctaa tgttcactgg |
| 156601 | gaaggagggt gtgggggttg agctagaatc caggtatctg atccgttagt ctgggtctct |
| 156661 | tacccctgca ctggcttccc cctcatgcca agctcatccc accggcacta cacatggaga |
| 156721 | aagacacaga cggagtgaag aagggcagag atagccgatg agttattggg cttcaagttg |
| 156781 | ggaagagagt ttctttagtg atgtgggctg ggtggagata ttggtgggga ggagggtctt |
| 156841 | gatgaacact tgccttgttt ccatttattt atttatttat ttatttattt atttatttat |
| 156901 | ttgcgacagg aatcttgata tattgcccag gctggtcttg aactcctgtg cccagacaat |
| 156961 | actccctcct tggcctccca gagtgctggg attataggca tgagtcactg tacctgacct |
| 157021 | gctttttaaa aaaaaatgac aactaatgtt gcaattaaca atcttttact tagtggaatg |
| 157081 | ttatacttaa attccagaaa ggattattgg gtcaagagta catataattt tgagagttac |
| 157141 | aactttatgg gggttgtatc aatgtgcctc ccccaccagc aaagtatgag agtgcctctt |
| 157201 | tctccactgg gtgtgatggc tcatgtctgt gatcccagca gtttgggagg ctgaggcagg |
| 157261 | agaatctctt gagcccagga gttcaatatc agcctgggca acatggtgaa accccaactc |
| 157321 | tacaaaaaac taaaaaatca gctgggcatg gtggtgtgtg cctgtagtcc caactactag |
| 157381 | ggaggctgag gtgagagaat cgcttgactc ccagcaggct gcggttacag taagctatga |
| 157441 | tcatgccact gcactccagc ctgggcaaca gggcaagaca ttgtctcaaa aaaaaaaaaa |
| 157501 | aaaaaagaga gagagagaga gtgcctgttt ctccacaacc ttgtcaaccc actatgccat |
| 157561 | taaactttgg ttttctgctc atcttacagg tggggaaagg gcagctcagt gcagttttca |
| 157621 | tctgccattg ttctgagatc tttccatatt gacctattga tatgttaaat taccaggtag |
| 157681 | taatccattc taggtgtaca ttacattgaa ttcagtctcc tactgatggg cattggaagg |
| 157741 | aatggggttc ttttatgatc ctgacattgc tgaaagccac aggccagtta tttagtagaa |
| 157801 | tactcctcca tttgggtttg tctgtttcct catgattcat tgaggttatg cgttttgggc |
| 157861 | acaaataccc tacatggtta tcccgtgcat cataatggga ggcatgtggt gtggggtttg |
| 157921 | ctcattcctg gtgacgttaa ctttgatctt tttcttgaga ttgtgtctgc caggtttctc |
| 157981 | cactacactt tcccctttgt aactagcaaa tatgttgtag agagagactg gagaactaga |
| 158041 | gatactctat tcatcagcac acttccacct atatgttcta gtagctgatg gaccttaaac |
| 158101 | atgtcttact atgttacagc tctttcacca acccttcaat acttttcagt ggtatcaact |
| 158161 | gtaaaaacca aacttcttca caatagcaca gaagaccatg cccctccttt gagccctcaa |
| 158221 | gccaaatctc tcccacacca ccccataaat gagctccagt cacatttctc atgttggggg |
| 158281 | tctttgctcc agcaactctt tctctgcatg cctgaaatgt tcttcattaa tcctctgcag |
| 158341 | cctagcactt tgtggccact tagatcacaa cctacaatcc ccccctaaaa tgacttctga |
| 158401 | ccactcaaac tgacaaaccc acccagctga tttctcatca cactactcta tgtccttgtg |
| 158461 | cagattttta tctactgtgt ttgttgtcag ccttttccct aacaaaacat actctcccga |
| 158521 | aagcagggtt gtgtctgtgt cattttatgc tacatcccca gggtctgggt cagtgcctgg |
| 158581 | caccgagaat gtgctcagta agttaaggaa tcgctgcatc cattctgtca gtcaacaatt |
| 158641 | ccttctgagc atccagtcca ctggcccatg ctggtctcag ccaacagaaa gggaattgag |
| 158701 | ttccttgcaa ggtctggtgg ggacagacag ttctgagaac cagtgactct tagaacaagg |
| 158761 | agatgaggct ttcacagagg gaagcacaga gtgtggtatt attctgctga agcccaaaga |
| 158821 | ggtcagggaa ggattccaaa ggagtaatgt ttgccaagca caaaacagca cagggaatgg |
| 158881 | cattccatgg acaggtctca gcaagtgcaa aggcttagag gtaatagaag tgcaagcaga |
| 158941 | attccagggt ggctaagaga ggttgtgtgg ggaatgggca gcagttaagg ccaaagaaga |
| 159001 | aacactggat ctgcattcct taggacggtg ccctgtgggc ccctcacaga gcttctctcc |
| 159061 | tgcaactgct aggtataaga aggcaggctt agaatcggac tgagatcagt agtttctgca |
| 159121 | gctgggaaga tgagtgaggt gagattgcat gtgtgatagg ctggcactca gggaagacct |
| 159181 | cagggttctc tcctccccac tctgaggccc ctctcctgct tcctgaagcc cagagtctcc |
| 159241 | acctctgtga gccaaacacc ttcctgttat ctgcctggtc ctagggtagc cttcgggttg |
| 159301 | gggttagaag gaatgccaat ggttaacctt gtggtattaa gcttccagct caggcagata |
| 159361 | ctctctggac tggttcaagc tgactgcctg actgtccctc cctgctggcc ctagcaccca |
| 159421 | gactccagat aggcacacca cctcaccctt cagagcaggg ctctgagact ccacaagtgg |
| 159481 | tgaggactta ccagaggaac tgaacttgac ctccaaccca accttccatt tgctggtatt |
| 159541 | attctgaagc cctcatggcc caggggcctg cctcaggtag caggtctcac tcctgcagtg |
| 159601 | gaggggccat aaagttcagt tcatgatcag cccccaagcc aggggcagcg gatgacaggg |
| 159661 | caggagttcc atctagagga gctcctgtgt tcaggcgttc ctagacacct gcctgccctt |
| 159721 | ctcccatcag cactggggta taaagaagga aagaggcctg aggtttccag aggcctctac |
| 159781 | ctgctgaggt cttaagagga ctcaaccctc tgggaaatac aaataaaatc ctcaaccctc |
| 159841 | caaacaactg aatggacacc tcttggccaa agagaccccg gaaaaacttt aaaatccaag |
| 159901 | tttcctggcc gtgatgatag gtcactcaca cctcagcaca cctgcttcct cacgaaccgt |
| 159961 | taccaggctt ctttcccaag agctaaacag aaaccagccc tgaaaaccaa gaacaggaga |
| 160021 | ctccttcact gatttcaatt tcaaccaatt ccgagactcc cttccctttc ctgattttga |
| 160081 | catgacagct gatcagctta caaaacattc ctggctgatc aatgactctc aaccatggac |
| 160141 | cagttctggc tggtttacag aggcagcaca caaagtgctt tgggtcctgt gtttcacatt |
| 160201 | ttgacataca gagcctaatt caactgcatt ttaatgtctc caccccgaag tgaatatggg |
| 160261 | acatatttaa catgttttat tggtacacat gtgtgcaact ctcatgaata ttcataaatc |
| 160321 | ctcttaaaac ttattaaata tatatgttta gccaactgat ttagtgtaaa acccctgtcc |
| 160381 | cttcaattcc tagcttgtag gttgcaaccc atcaaaagaa ataaagctct cttttccaaa |
| 160441 | tgtaaagatc ttatgatttt aagccaatat aattgaagac aagagtagga tccatggagc |
| 160501 | agcccaggtt cccccagcct aagtgaatgc acaggtgcca ggtggagcca ttgacagctc |
| 160561 | atttgtctcc ctgacagctt tgggaggaag gtgggtaagc tctccccgat ctaagatctc |
| 160621 | tcacttttga attgagatct cgaggacttc atttttttac cctgatcccc tccatccgga |
| 160681 | ccctgcaagt gccatctttg ttggtcccag gtttctagat ggggaggttg ggggcagtta |
| 160741 | agcctgactc atccggcgca tcagcccatc aggtttggtg aggatgcttg ccctctagtg |
| 160801 | gcacacgaag gaatgtctta tgcttttctg gggaatctag attctaagga gacagtccca |
| 160861 | gaccagctgc catcaggaac atgcatgttt cagaattatg agaaaaagtc ttgtgaatat |
| 160921 | tttggatcct ggaaaaaagc taacctggaa caatataaag atttataggc caaaatggag |
| 160981 | aacttttggc agaatgtctg acatgcctaa attagtttat ttgcatgcac aatagggttc |
| 161041 | aggacctctc agaagcaatg ggaggtcttt tttttttttt tttttttgag acagagtctc |
| 161101 | actctgtcat ggggagtgga gtggagtggc acgaacatgg ctcactgcag cctcgacctc |
| 161161 | ccaggctcaa tcatcctccc acctctgcct ctcaagtagc tgggattata ggcatgcacc |
| 161221 | accacacccc actaatttct ttattttcaa tttttaattt tttatagacc atgctggtct |
| 161281 | caaattcctg gactcaagga atcctcccac gggagaccat ttttcagtgt tattatgaga |
| 161341 | gctctaaaca cagttgggag tctcagataa cctccttaaa tgagactaat tcaaaactga |
| 161401 | aggagtctaa ttcaaaactt gaccagcata ttcaaaccta tctgccacta ttggacaggt |
| 161461 | agctgaagac actgcaaaaa gccttatagc ccaacaaatt gaattccctg gctcaagtag |
| 161521 | taatagataa tttaattgct tcagattttc ttttacccaa acaaggaaga gtctgtgcag |
| 161581 | tggcccataa cacctgttgc acttacatca acacttcagg tgaagtagaa actcatatag |
| 161641 | ggttgggcac agtggctcac acctataatc ccagcacttt gggaggctgg ggagggagga |
| 161701 | ttgcttgagc tcaggagttt gagaccagcc tgagcaacat ggtgaaaccc tatctctaca |
| 161761 | aaaactgcaa aaattaacca ggcatggtgg cacatgcctg taatcccagc tatttgggag |
| 161821 | gctgaggcaa gaggatcact tgggcctggg aggcataggt tgcagttagc ctagatcacg |
| 161881 | caattgcatt ccaaactgat aaagcaagac tctgtctcaa aaaaaaaaaa agaagaaaaa |
| 161941 | ctcatacaga aagaatttcc aaacaagcta aatgattaca agaaataaaa ctactgatcc |
| 162001 | tatcaatgat ctgttcagtt ggctttctac caaacagaaa ttcctttcaa gatgctattg |
| 162061 | aaggctgggc gtggcggctc atgcctgtaa tcccaacact ctgggaggct gaggcgggtg |
| 162121 | gatcatctga ggtcaggagt ttgtgaccag cttggccaac atggtgaaac cccgtctcta |
| 162181 | ctgaaaaata caaaaaatta gctgggtgtg gtggcgacac ctgtaatccc agctacttgg |
| 162241 | gaggctgagg caggagaatc gcttgaacct gctaggcgga gattgcagtg aaccgagatc |
| 162301 | acgccactgc actccagcct gggtgagagt gagactccat ctcaaaaaaa aaaaaaaaaa |
| 162361 | aaaaaggtgc tattgaagtt tttgttataa ttatagtctc catcaaactt ttcttcataa |
| 162421 | catttaaatt acttataata tgggacaggc atgggggctc aagcctgtaa tcccagcact |
| 162481 | ttgggaggtg ggcagatcat ttgaggtcag gagttcaaga ccagcttggc caacatggcg |
| 162541 | aaacactgtc tttactaaaa atacaaaaat tagccgggca tggtggcatg tgcctgtaat |
| 162601 | cctagctact cgggaggctg aggcaggaga attgcttgaa cctgggaggc ggaggttgca |
| 162661 | gtgagccgag atcacaccac tgcactccag cctcggcaac agagggatac tctatctaaa |
| 162721 | aagtaaatat aaataaataa ataacttata atatgtataa ccaactgctg taagtctgct |
| 162781 | gctaaaacca gaattatgct ggctcaatgc atagaactta tagacaagtt aaattggtag |
| 162841 | ccctaccttt tggcctttat attgcttgat agaccttagg ggttgatgag tacctgccca |
| 162901 | cctctatttc tgtctggcca agatgttcaa ttggctgtaa gtctcttggc cacaagggtc |
| 162961 | ccaccaaggg actggatgga tctggagcag gtagcctcag catcctggca acgacatggt |
| 163021 | acaaacaatt tggccattga tgctgactgt ggcagatctt ggctaaaagg aagaaatgtg |
| 163081 | gaataaaaag aaaatccaaa gacccctcaa ctgactgaac aaacccctct tggccaagga |
| 163141 | gaccccagaa aaactttaaa atctaggttt cctggctatg ataagacagg aggctggtca |
| 163201 | cacctcagta taccctcttc cttactgtta ccaggctttt ttcctaagag ttaagcagaa |
| 163261 | tcctgtcctg gaaaacagag aatggaagac tcctccgctg acttcagctt caactgcctg |
| 163321 | atgccatggc cagacttcac tctctttttg tgatttgaca cgacagctga ccagctcaca |
| 163381 | aagctatcct tcctgatcag ttcctgttaa ccatgggctg gttctggctg gtttacagag |
| 163441 | gctgcacaca aagtgccttt gtgtcctatg tttcaccttt tgatgtgtat ggcctaagtc |
| 163501 | tgcattttaa tgttaagtct cagctgggta tggtggctca atgcctgcaa tcctagcact |
| 163561 | ttgggaggcc aaggcaagag gattgcttga gcctaggagt tcaagaccag cctaggcaac |
| 163621 | atggtgagac cttgtctcta tttttttaat taaaaacaaa atacaaatat aaaaataatt |
| 163681 | actgttaact ctccacccca aagtgaacac aggatgtatg taacatgtat gtttgcttag |
| 163741 | tatacatgca tgtgactccc tttcatgaat attcatagct cctcctataa cttattaata |
| 163801 | agtatactag ctaacctatt tagcataaaa ctcctgtccc acctctcctc cctcaaagtg |
| 163861 | cctgctttcc atctcagcca gaggctccac ttcccagcct gcaggttaca acacaatatt |
| 163921 | agtaaaagtg ttctttccaa gtgtatacat ctggtgattt taagttgata cttcccaggt |
| 163981 | tgtccaagat tcaggtacag ctcactattg caggatacaa gctgggatct cctgggagtt |
| 164041 | ggtctctttg caaatcgtat tatctctgta tcacctttta tgaaatccta aaagaattta |
| 164101 | aatctgaaac atgtgactcc aaagattttg aataaggaat tgtgaaccca tggtaaactc |
| 164161 | ctggcttaag ggtgtccaat tttttggctt ccctgggtca cattggaaga ataagaaagt |
| 164221 | ttaagaaagt ctgtgaattt gttttggggc acattcaaag ctgtcctggc cacatgaggc |
| 164281 | ctgagggcta taggttggac aagcttctag ctggctattt ctgcattttg tccaaatcca |
| 164341 | tcttttaatt attatcatta ttattattat tatttgagac ggagtctcgc tctgtcaccc |
| 164401 | aggctggagt gcagtggcgc gatctcagct cactgcaagc tctccctccc gggttcacgc |
| 164461 | cattctcctg cctcagcctc ccgagtagct gggactacag gcgcctgcca ccacgcccgg |
| 164521 | ctaattattt tgtattttta gtagagaagg ggtttcaccg tgttagccag gatggtcttg |
| 164581 | gtctcctgac ctcgtgatct gcccacctcg gcctcccaaa gtgctgggat tacaggtgtg |
| 164641 | agccaccgcg tccagctgaa acataatttt tctctttttg gtttcacatt tttactaaag |
| 164701 | acaaatcatg gtaagactga tttgctttat tatacttggc ctgattattt gtgtaacgtg |
| 164761 | cagcaagaat acttactttt cacataggct ttttaagttg gctttgatgg aactttgttt |
| 164821 | tgtagaagaa tctcagatta gactttttct tttaaggccg ggctcagggg ctcactcctg |
| 164881 | taatcccagc actttgggag gctgaggagg gaagatcaca tcaggagttc aagaccagcc |
| 164941 | tggccaacat ggtaaaaccc cgtctctact aaaaaacacc aaaaactagc caggcatggt |
| 165001 | ggcaggtgcc tgtaatccca gctacttggg aggctgaggc aggagaatca cttgaacctt |
| 165061 | agaggcaaag gttgcagtga gccaagatca caccattgca ctccagcctg ggcgacagag |
| 165121 | cgagactctg tctcaaaaaa acaaaacaaa acaaaacaaa actttttttt taaagccgag |
| 165181 | tttggccatg cggttgtacc atcaaatacc tatgagttgg gtgaattcct cttgagaacc |
| 165241 | caagatgatt tggggctcct gggtctgtca gagagtgaca ttctttactt gccacaggtc |
| 165301 | agaaaccctg ctcaggatct gtgtagaaaa ggtatgaagt tagttttccc aaggggctct |
| 165361 | tatcagctct ataagtcaag tttgattcct taaaggaaag cacaccattc cagtcaaagc |
| 165421 | cttggtaaaa taactggttt ctccaattgt gtcctgttac aaaagaaaac agattctggc |
| 165481 | tgggcacagt ggtcatgcct gtaatctcag tactttggga ggctgaggtg ggcagatcac |
| 165541 | ttgaggtcag gagattgaga ccaacctggc caacacggtg aaaccccgtc tctactaaaa |
| 165601 | atacaaaaat tagctgcttg tgttgtgtaa ttgggtaata agagatttta aagaattttt |
| 165661 | tttgtagagc accatggttt aaagtcagct taattaaaat tagatattca agctctaaca |
| 165721 | gcctgggact ccttggggaa aacaggaggc gccagagaca ccattttgga aaaaaaccct |
| 165781 | gttttcctct tggaacccca ggaattgaaa gctgataaat tcctctcaaa atttaaggct |
| 165841 | ttgttctgtt ttggattgca gtatctgaag tttttgactt ttggtctatc agaaattata |
| 165901 | tcgcattatg agagagtttt ggtgtgtaat aactaggtag gaaatatact ttaaggaatg |
| 165961 | gctaatggaa attatagatg atcacgtagc tctttgcatg tttggattag agaagcatgc |
| 166021 | tcttggccac ctggaaggta tggaaatacc tttttttttt tttttttttt ttttgagaca |
| 166081 | gagtctcact ctgtcacccc ggctggagtg cagtggcaaa atctcagctc actgcaatct |
| 166141 | ctgcctcctg ggttcaaagg attctcctgc ctcagcctcc caagtagctg ggattacagg |
| 166201 | cacctggcac catgcccagc taatttttgt atttttagta gagacggggt ttcaccatgt |
| 166261 | tggccaggct ggttttgaac tcctgacctc aggtgatctg cctgcctcgg cctcccaaag |
| 166321 | tgctgggatt ataggtgtga gccaccgtga ctggctggaa atatcttctt atctcccact |
| 166381 | gagagataag acttccacag aagatgggct gattccccct tttttgggga gggatccagg |
| 166441 | gtctggtata aaatgggatc ttcattttgg gggatctgtt ttgccttcca gctgtgcctg |
| 166501 | cttattaggg ccaaaggtac ttgggagtcc cagctatttg ggagactcag gcaggagaat |
| 166561 | cacttgaatc caggagatag aggttacagt gagctgagat cacaccactg cattccagcc |
| 166621 | tgggtgacag agttagactc tgcctcaaaa aaaaaaaaaa aaaaaaaaaa gaaagaaaga |
| 166681 | aaaaagagac agattattta gattatttat ttacttactt tttttttgag atgcagtttt |
| 166741 | gctcttgtca cccaggctgg agtgcaatgg cacgatctcg gctcactgca acctctgcct |
| 166801 | cccgggttca agtgattctc ctgcctcagc ctcctgagta gctgagatta caggcacaca |
| 166861 | ccaccacacc cagctaattt ttatttattt atttttagta gagacagggt tttgccatgt |
| 166921 | tggccaggct ggtcttgaac tcctgtcctc aggtgatcca cccacctcag cctcccaaag |
| 166981 | tgttgggatt acaggcgtga gccactgcgc ccggccaaga aacagattct tattgcactt |
| 167041 | atgcaaataa ctatattgct ataagttaag aatactcaca cctagtttcc taattctgga |
| 167101 | aaaatcaggt agaaaaaaac aaatatgctc caaatatgtt gccagaagta tactttactg |
| 167161 | aattgttttt tttttttttt ttgagacgga gtctcgctct gtcgcccagg ccggactgcg |
| 167221 | gactgcagtg gcgcaatctc ggctcactgc aagctccgct tcccgggttc acgccattct |
| 167281 | cctgcctcag cctcccgagt agctgggact acaggcgccc gccaccgctc ccggctaatt |
| 167341 | ttttgtattt ttagtagaga cggggtttca ccttgttagc caggatggtc tcgatctcct |
| 167401 | gacctcatga tccacccgcc tcggcctccc aaagtgctgg gattacaggc gtgagccacc |
| 167461 | gcgcccggcc tactgaattg ttaaaaggtg taaatagctc aaaatttttc ctgactctga |
| 167521 | aaacaaaaca aagtttcagc agcattttaa gcaaagtcaa aaacatttct tcagtcttct |
| 167581 | atttgttcat tccatgcagt taactcctgt cctgtttaat cgtcatgaac atttcagctg |
| 167641 | tccatgagtc ctgaaagttt ttcctctatt ctgacgtcac aatctccaaa cttatcagaa |
| 167701 | acctgcattc aagagaacct gtcaaaatcc tatagttgat tataaaccac cttttgaaga |
| 167761 | ggatcaaaat aagaaaacaa ttgtctgtgg ttgacagaag tcttaggaca gccactattt |
| 167821 | aagccacaat tgaaaaggaa atgtgggggt gggcacggtg gctcacgcct gtaatcccag |
| 167881 | cactttggga ggccgaggca ggcagatcac ctgaggtcag ggatttgata ccaacctggc |
| 167941 | caacatggtg aaaatccgtc tctactaaaa atacaaaaat tagctgggga tggtggtggg |
| 168001 | tgcctgcaat cccagctact gagagtctga ggcaggagaa tcgcttgaac ctgggaggtg |
| 168061 | gagttttcag tgagctgaaa ttgtgccact gtactacagc ctgggtgaca gagtgagact |
| 168121 | ctgtctcaaa aaataaaaaa gaaaagaaaa ggaaattttg gttagttctg tgacaacaat |
| 168181 | tttacataac aattatgact attaataaca tacactaagt tatcaaggaa ttatgggtgt |
| 168241 | tttccataat tttggaacat gtccaataac atatttatgc aaatataggc caaagaaagc |
| 168301 | caaatactat ttcacatttg acgatgcttc ctttatggat tttatactga ataagccaaa |
| 168361 | tttcaccttt acgttagtgt actattaatg ttaaacccaa ttctttaata aaacctttta |
| 168421 | gacaaattta tttaatctta atcagtttga ccataaagta agattcttat aaacttttgt |
| 168481 | ttcctcacct ccccagaaaa gtggatccta aaccttttat aaccctttac aatttttgtg |
| 168541 | aacgagcaga ttaatgctct aagaaaaacc tgttgtgctt ttattccaat gtttgattta |
| 168601 | tagaaaaaat gaatacccct ttaactttag ccaacatgtt cacacagaat ttcttttatg |
| 168661 | acattaattt ttcacaaacc tcccacaatt tgttcaagcc ttcagcttta tctcatctaa |
| 168721 | aacaatcctt taacccttta atctaggcag aaaaatccac attcccatga cttcttataa |
| 168781 | tcttttacca aaaacacatt tcactttcct tacacacctt gcatgtaaaa ctgatgttat |
| 168841 | ttcccaaaga ttactaaagg catataaact aaaaggcatc acagttttta tttttctaat |
| 168901 | aaaatatttg atttaagctc ttattatttt ttaacctatt aatcaaagct cttttatatc |
| 168961 | tcacacacac aacatatata aatacacaga ccagaagatc cagtagttgt aagaattttc |
| 169021 | atttgccagt ttctttttct ttcttttttt ttgaatcgga gtcttgctct gtcgcccagg |
| 169081 | ctggagtgag gtggcttgat ctcggctcac tgcaagctcc gcctcccagg ttcacgccat |
| 169141 | tctcctggct cagcctcctg agcagctggg actacaggct tctgccacca cgcctggcta |
| 169201 | agtttttgta cttttagtgg agacggggtt tcactgtgtt agccaggatg gtctcgatct |
| 169261 | cctgacttcg ttatccacct gcctcggcct cccaaaatgc tgggattaca ggtgtgagcc |
| 169321 | accgcgcccg gccatcattt gccagtttct taattggatt actgacttta gggtggagcc |
| 169381 | cttgggggaa caaggtcagg aaagcatgca gtttctacag cctaataagc aggcaagctg |
| 169441 | gaaggcaaga cagatccccc aaaataaggg tcccatttta taccagatcc tggatccccc |
| 169501 | ccaccaaaaa aggggaaatc agcccatctt ccgtggaagt cttatctctc agtgggagat |
| 169561 | aagcgggtat ttccatacct tcgaagtggc caagagcatg cttctctaat ctaaatatgc |
| 169621 | aaagagttaa gtattcacca ataacttcca ctggccattc cttaaagtat atttcctacc |
| 169681 | tagttactac acaccaaagc tctgtcataa tacgatgtaa tttctgatac caccaaaagt |
| 169741 | caaaaacatc agatactgca atgcaaaaca gaacagagcc ttacattttg agaggaattt |
| 169801 | gtcaactttc aactcctggg gtttcaagag gaaaacagag tggtattttt tttttttttc |
| 169861 | caaaatggtg tctctggcac ctcctgtttt tcccagggag tcataggctg ttagagtttg |
| 169921 | aatatcctgc tttaatgaag ctgactttta accatagcac tcttaaaaat aaaaatcctt |
| 169981 | taatgtctct tattacccaa cttcagccat gccaaactac caatatttct ggcttctgaa |
| 170041 | ctttaccaaa ggcaaactcc caggtactcg gagaaaaaaa attcaagaca gtttgtggag |
| 170101 | ggaaagagaa tctacaaatg ttcagacaga tctcaaacta gaaaggactc attccctaag |
| 170161 | ctggggattg aaccctgaac ctgggctgcc actgtgaaaa gacagtctta gctgctgagc |
| 170221 | tacagcattg ggcagtctcc attgcccttc ccagaaggag gctagagtca ccaattttga |
| 170281 | gcttgcaaag gcttttatct gatcaagata atttttagga gtaactatgg catgaacccc |
| 170341 | aaaactcctg tctgctggat ggtagaaacg aagagaaagt atcgccacgt ggttacaagg |
| 170401 | tgaagctccc aaggacataa aacaagacga aagagaaatt tcatccagct tttcagggac |
| 170461 | cttcagcaaa gtttataact gatcagtttg cttggccatt ttgaacagcg ggcttacagg |
| 170521 | cgtcatagtg agagacagga ctagctggat atcctaggcc tactaagaat ccctaagcct |
| 170581 | agctgggaag gtgactgcat ccacctttaa acacggggct tgcaacttag ctcacacccg |
| 170641 | accaatcagg tagtaaagag agctcactaa aacgctaatt aggcaaaaac aggaggtaaa |
| 170701 | gaaatagcca atcatctatc gcctgagagc acactgggag ggacaatgat aggatataaa |
| 170761 | cccaggcatt caaaccagca agggctaccc tctttgagtc ccctcccttt gtatgggagc |
| 170821 | tctgttttca ctctattaaa tcttgcaact gcacactctt ctggtcagtg tttgttacga |
| 170881 | ctcgagctga gctttcactt gccgtccacc actgctgttt gccgccatcg cagacccgcc |
| 170941 | gctgacttcc acccctccag atctggcagg gtgtctgctg tgctcctgat ccagtgaggc |
| 171001 | acccattgcc gctctggatt gggctaaagt cttgtcattg ttcctgcacg gctaagtgcc |
| 171061 | cgggttcatt ctaatcgagc tgaacattag tcgctgggtt ccacagttct cttctgtgac |
| 171121 | ccacggcttc taatagagct ataacagtca ctgcatggcc caagattcca ttccttggaa |
| 171181 | tctgtgaggc caagaacccc aggtcagaga acaagaggct tgccgccatc ttggaagcgg |
| 171241 | cctgccacta tcttgggaac tctgggagca aggaccccct ggtaacagta ggcttgcatt |
| 171301 | ctatcctaag gtacccttct ttatggcaga acaatataga cacacaaagc acaccagatt |
| 171361 | cgttacagct taaggctagc ctcacaagtc ttttttctca ttaaccaaaa cttgcctgtg |
| 171421 | gtaaggtggg gaaagtgaag agctcaggaa gaccagagaa agactcacct atcccagtga |
| 171481 | cactgaatca aaagttcagg cagccgctca ttggttaaga agggatcttt tccaggagtt |
| 171541 | gcatcagctc ttaagtgtcc ccgtttgggg agcaaaaagc tctcatgtcc caggatcctg |
| 171601 | tacatacaca attctgtcac ccacagccat cagcaaagat ttcaaggtag attaatccaa |
| 171661 | agagaatagc aattaatatc ccatggtgct aaatccgttc ttagccaaga ggaactttac |
| 171721 | tctgagaggg gcttctaact ccctaaatct tagaagggac tctaacactc ctgagttggg |
| 171781 | cctcaaacca aagttcagtc aagtgtcctt gccttttatt aagaggggcc tttaacccac |
| 171841 | tctgtcttag gagagacttt aactccccta agttgagcct ctaacccaat tccatccttt |
| 171901 | acctgggtac cccgccactt accctagata gccaattggt gctgcagtct atttcctttg |
| 171961 | ggtagggtgt ctcctcaggg ttcgacagga agatgttacc agaaagggat ccagatccag |
| 172021 | accccaagag aaggttcttg gatcttgtat aagaaagaat ttggggtgag tccatagagt |
| 172081 | aaagtgaaag caagcttatt aaggaggtaa aggaatacag aatggctact ccataggcag |
| 172141 | ggcagtggct tgagctgctc cactaaggat acttattgtt acttcttgat tatatgctaa |
| 172201 | agaaggggtg gattattaat gagttttcca aggaaggggt ggccaattcc cgtaactgag |
| 172261 | ggttcctctc ctttttagac catttagggt aacttcctga tgttgccatg gcatctgtaa |
| 172321 | tcacggcact ggtgggagtg tcttttagca tgctaataca ttataattag catataatga |
| 172381 | gtgatgagga tgaccagagg tcactttggt cgccatcttg gttttggtgg gtttgggctg |
| 172441 | gcttctttac cacaggctgt tttatcagca aggtctttgt gacctgttat cttgtgctca |
| 172501 | cctcctagct catcctgtca cttggaatgc cacacctcct gggaatgcag cccagtaggc |
| 172561 | ttcagcttca ttttacccag cccctattca agatggggtt gctttggttg aaacgcctct |
| 172621 | gacagaccca ccagaggagc ccttcagttt gtattaatat gatatgtgac tacttatttc |
| 172681 | acagacactg tgtgtcaaat gtcggtacaa tgccaacaac tcactttctt ggttgttgag |
| 172741 | tttccgcatt acacaaataa ggaagcaggc ccagaggaga gcctgggaaa tgaagttgga |
| 172801 | gagacccatc ctggggttgc ttgatttagg gatttagact gggaatgact cctccataga |
| 172861 | tctgaaagaa gaaactgcac actgttcata gtggcttctt ttctgccagc cctaaacagc |
| 172921 | tcaagaaagg agagtctctc agttatgagg ctgggtgtga agcatttttt ttttctttga |
| 172981 | gagaggtctt gttatgttgc ccaggctggt ctccaaatcc tgggctcaag cgatccttcc |
| 173041 | accttggcct cccaaagtgc tgggattaca aatgtgagcc accacatccg gcctgaagca |
| 173101 | tcttggttca tgcatctagc aaaccttcag tctgtgtctc tcaatcccac tggacatgag |
| 173161 | gtctgcctat aacaattatt tggctacaac ccctttaata tcctgaactt agctaaacag |
| 173221 | ttattttaaa aatccatatt caaatataac taaagaagaa acagaggaaa agtaatttgt |
| 173281 | aatatggatt tctatttata ttcgttttga ctccattagc agatagcagt cactgccctg |
| 173341 | tttgcacttg ttttgaatca gtgagcctgt agctgccaag tgcagactaa caagaggtaa |
| 173401 | ataacctgta taccttgagc agctctgact ttaacttatt cttttatttt ttatattttt |
| 173461 | attttttttt atttttatct ctttttagac atgaggtctc actatgttgt cccagttgac |
| 173521 | ctcaaactcc tgcactcaag tgatcctccc accttggcct ccaaagtact gggattatag |
| 173581 | gtgtaaacca ctgtgcctgg gcttttatat attttttaag tgcacatttt aatgtttagc |
| 173641 | ttgtcagcct taagtaacga gatccagaaa gcttgaggat agctacacag aagcatagat |
| 173701 | tcaagttgtc ctgaatatac acttcgattc acagcagtta caagtgagtt tttaaggaaa |
| 173761 | cagaatagtt cctaagttgt ttatcaagaa tttaaaataa aataacataa gcaattcatt |
| 173821 | ggctatacat tgttttttgt atcacaaatt ccaggaacag ataacgggtg aggcagctag |
| 173881 | tcagggacaa aacgccttgg aacaattgtc tctgggcatg ggtggggagg gtgtgactga |
| 173941 | agttccatac tcatgccttt ctgggcctga taaattgggc atgtctcaca tcatgagact |
| 174001 | gctctgagct atttttcttt tctcaagctt tataccaaat tctaggccgt gggctttcat |
| 174061 | tcattcaata agtatgtatt gagtgtttat gtaatcggaa ctcttctggg gttacaattt |
| 174121 | aacttacttt ctcacctccg aatggttatc acctcaacga ggactaaatc caatgatttt |
| 174181 | tttttatctt gccaaaattc ctatctaagg ggtctgggga gtcatgccct acaaatcata |
| 174241 | aattctcatc agatgggttt tatttaaccc tatatatcat gacttacttt ccaacctgac |
| 174301 | tctggcataa catgacgaga caaggaagaa aagcaaaata ttttaatatt tttacctcaa |
| 174361 | aacatgtttc tttgccatat cttgaaatgg ccctgctact gtcctttgtg ggggaaaatt |
| 174421 | ttcatctgta aagaatctct attaacatag ctggatcttt ttctaccagg cccttccaat |
| 174481 | cataaagaga ttaactaagt gtagcacctt ttatagatct gaataggaaa cacttgtcat |
| 174541 | ctgttgtttc taagggcagc cactataaga cttcaaaaga accttggtct ccacagtctt |
| 174601 | ttatcttaac atttcttttc tatcaacccc aggtctttag acaaactcaa ccaattgtca |
| 174661 | accataaaat gtttaaattt acgtatagcc tgaaagccct gccaccccac tttgaattgt |
| 174721 | cccaccttcc tggaccaaac caatgatttt tttttttttt tttagatgga gtctccctct |
| 174781 | gccgcccgtg ctggagtgca gtggtgtgat ctcggctcgc tgcaacctcc gcctcctggg |
| 174841 | ttgaagcaat tctcctacct cagcctcctg agtagctagg attacaggtg cctgtcacca |
| 174901 | cgtccagcta attttttgta tttttagtag agacagggtt tcactatgtt ggccaggctg |
| 174961 | gtctcaaact cctgacctca agatcagcct gccttggcct cccaaagtgc tgggattaca |
| 175021 | ggcgtgagcc accgtgcctg gccaatgtat tccttaaatg catttgattg atgtctcatg |
| 175081 | cttccctaaa atgtatgaga catccttctc ccctgattgt tttcaaaagc agtgatccat |
| 175141 | tgaggtgctc atttttgtaa ccccagttac tagaaaagtg cctgggtaag agtatgtagg |
| 175201 | atctctataa aagctgaatt aacgaatttt gtaatgactg cacctccaga caggagctgt |
| 175261 | cttccgggct tccacagtct ctgacagccc tctcccacaa agagtttacc aacagcaagg |
| 175321 | actttcctgg atgacttcca ctgggttggg gattaaggat tgaaagggga gaggctgggt |
| 175381 | gtggaatatt ctggctgtgc tggctgtgga cttagtcctg tgtcttcccg catccagtgt |
| 175441 | agtctctgga gaaagaatgc ctgagcttta ccagcaccac ccaggaagcc cataagaata |
| 175501 | ttcccatcta tatggattca actggaacca ctgtggagag atggtacctg cctgcaaacg |
| 175561 | gcactttatc caggacacct gcctttacga gtgacccccc aacttggggc cctggatcca |
| 175621 | gcaggtacgc atggcttcct ggcatccaag agctagcaga ggagctgaat tttccaggcg |
| 175681 | tctctgcagg cagcaacccc agctccagtt ctattcaggg ctgggttcct gggattcttg |
| 175741 | agcctgagcc cttcttttct accaaaatct cccaggtgga tcagagctgg cgcaaagagt |
| 175801 | gggtgctgaa tgtgcccctg tgcaaagagg actgtgagca atggtgggaa gattgtcgca |
| 175861 | cctcctacac ctgcaagagc aatgggcaca agggctggaa ctggacctca ggtgagggct |
| 175921 | ggggtgggca ggaaaggagg gatttggaag tgaaggtgtg tgggtgtgga acaggtgtgt |
| 175981 | gacattttgg ggttgtaggg ctggcagaat cagagaccct ttggggccca gtggctaaag |
| 176041 | gtcttccctc ttccctacag ggtctaacaa gtgccaggtg gcagctgcct gactaccttt |
| 176101 | ccatctctac tttctcacac ccactgctct gtgcagtgaa atctggactc actcctacag |
| 176161 | ggtcagcaac tacaaccgag ggagcagccg ctgcatccag atgtggttcg acctggccca |
| 176221 | gggcaacccc aatgaggagg tggcaaggtt ctatgctgca gctctgagtg gggctgggcc |
| 176281 | ctgggcagcc tggcctctcc tgctcaacct ggccctaatg ctgctgtggc tgctcagctg |
| 176341 | acctcctttt accttctgat acttggacat ccctgccctg tttagcccca cagctcccaa |
| 176401 | ctatttggtt cctcttctat ggtcttgtct ctgacagcca ctttgaataa accagacacc |
| 176461 | acacatgtat cttgagaatt atttgggtat gaatgggaat gtggctgttt tgtttcccat |
| 176521 | ttcttattga ttgaagccag ttagactggg ctagttccca gctctgatgc ttgctatgaa |
| 176581 | ctagcctgat acttaagtat tcttctaagg taggagacat ttgtagctct cgattttatt |
| 176641 | attcactata gctccaattt agagccaagc ccaggcattt ttcttttttt gagacagggt |
| 176701 | ctcactctct cacccaggtt ggaatggagt ggcatgatct cagctcactg caacgtctgc |
| 176761 | ctcgtgggct caagtgatcc tcccacctca gcatcccaaa tacttgggac tacaagcatg |
| 176821 | tgccaccaca cctggctaat ttttcaattt tttgtagaga ggaaatcttg caatgttgtc |
| 176881 | caggctggtc tcaaactact gggctcaagc aatcctccta cctcagcctc ctgagtagct |
| 176941 | gggtctacag gtgtgagcct ccacattcag ctaatacata taatataaaa catgtattat |
| 177001 | atatgtaata gtagagacag ggtctcacta ttttacccag gttgttctca aactcctgcg |
| 177061 | ctggagtgat cctcccaaaa tgctgggatt acaggcatga gtcactgcat ctggccacaa |
| 177121 | gtcatgttta tttcttgctc aagctacaat gctgaatgtg ggttatcagg gggattctgt |
| 177181 | ttatttctca ttcctggact acattagcag ccagggggtt ctgcttctca ctcaagaacc |
| 177241 | cacgctgatg gaggctccat cttccatgag acaggaagag ggaacttggc aaattccata |
| 177301 | aggctcttca aatttccacc catcacttat ttcattggcc aaatcaagtc atgagcatgc |
| 177361 | ctaaattcaa gaaagccagg aaaacataat cctatcatgt acttgcaagg agaggggaat |
| 177421 | cacaaggttt gcaacagacc taatcatgag taaaaagact tcattatttt ctcgtagatt |
| 177481 | tctgtttctg gcatggtcat agttacaaat ttaatttttt ggttagatcc tagtaagaag |
| 177541 | gaggaccaac tctctataca tatgcaaata actacatcaa cctgaaaagt ggagcaatcc |
| 177601 | actgatgacc gccctggcta gtcttgtaaa caaggccaat tgatgccttt tcgcccttca |
| 177661 | tttatttcta tttattattt ttattttttg agacagggtc tctttctgtt gctcaggctg |
| 177721 | gagtgcagtg gcacagtcat ggctcactgc agtcttgacc tcctgagctc aattgatccc |
| 177781 | tcaactttag cctcccaagt agctgggact acaggcacat gccaccatgc tgggcaaatg |
| 177841 | ttttgtattt tttttgtaga gatggggttt tgccatgttg cccaagctgg tcttgaactg |
| 177901 | ctgcgcccaa gtgattcatt cacccacctt gacctcctaa agtgctggga ttagaggcgt |
| 177961 | gagacacctc tgccagccca ctgatttctt ttttgaaccg aaagcatagt tgatcaatgc |
| 178021 | aaaagctaat aattggatat taacaaaatt caaaactctt gcactttaaa agacaccctt |
| 178081 | aagaaaatta aaaaacaagc aacagactgg aaggaaatat ttgcaaacac atctgataaa |
| 178141 | ggatctgtat ccataaaata caaagaacac ttacaactca gtaacaagaa ggcagacaac |
| 178201 | ccaatcaaat aatgggcaaa agaaatgaac aaatatgtca ttaaagaagg tacacaaatt |
| 178261 | atgaatagtt acacgaaaag atgctcaaca ttattaatca ttagaaaaat ggaaactaaa |
| 178321 | accatagtga tagacggttt tatacctact agaatggcaa taataataat gataggcaat |
| 178381 | aacaagcatt ggcaaggatg tggagaaatg ggaaccctca cacagtgctg gtggggatgt |
| 178441 | aaaatggtac agcccttcgg aaaacagtgt ggcagtttct caaaaagtta aatgtaaaac |
| 178501 | tgccatacaa cccaaaattc cactcccagg tgtctactca ggagaaacga aaacaaacat |
| 178561 | aacaaaactt aaatatgaat attaataata atcatattta tgtatgtaaa tatgatgtat |
| 178621 | catttatgta tgtaaatatg atatatcata ttatgtatca ttatatataa tagccccaag |
| 178681 | ctggcaataa cctaaatttt tagcagctgg tgagtagata gacaaaatgt ggtgaatcca |
| 178741 | tacaatagaa gactgtgcag caatgaaacg tgatgacaca tgctatgtcg tggatgagcg |
| 178801 | tcaaaaacat tatgttaagt gaaagaagcc agacctaaga ggcttcatgt ggcatgattt |
| 178861 | cacttacatg aaatgtgcag ggaaggcaaa tttatagagg cagaaagtag attagtagtt |
| 178921 | gcctggggct ggaagtgaca acagggatta attgtaaatg ggcatgaggg gggataagaa |
| 178981 | ttgaggggat gattctgagt ttctgtgggg tgtatccatg agggaaagta ttctaaggaa |
| 179041 | attggcatgt ggctccataa aaatctatca tctgagacaa actccattcc catcatgaac |
| 179101 | tttaaaatat tggttggaag ccaagcatgg tggctcatac ctgtaattcc agcactttgg |
| 179161 | gaggccaagg caggtagatc acttgagcca gggattctag aacagcttgg gcaacatggt |
| 179221 | gaaaccccgt ctctacataa aaatacaaaa aatttagccc agtgtggtgg tccacgcctg |
| 179281 | tagtcctagc tatgcaggaa gctgagatgg gaggatctct ttagctcagg aggtagaggc |
| 179341 | tgcagtgggc tatgactgtg ccactgcagt ccagcctggg ttgatagagt gagaccctgt |
| 179401 | ctcaaaaaaa aaaaaaaaaa aaaataggaa ttgctggatg gtaatgttgt aggagtttct |
| 179461 | cctcagttca gccaaagaca gggtccttgt cacctggcca tgaaatatta ggcttgcaga |
| 179521 | cactttgaag ggtgagaaaa atggaattta ttgggcaaaa aaaaaaagcg gggaaacggg |
| 179581 | gaccctcggc agagtgagag tcctgctagt acacgcttcc cacctctcag gttgaatccc |
| 179641 | aggttccacc ctggaagagg aggggccagg ctcctcagca cgaacttcca gaggctccac |
| 179701 | cccagtgcac attcctccca gtgtgcgggt ttgtcagagg ttctctgggg acccctttac |
| 179761 | acttggctgt ctcattcccc cctctcaaga agtacatcaa actactgtta gaacaaggat |
| 179821 | aggggtgagg acgaagaccg atctaaactg cttcctgcta acagggagcg gtgttttggg |
| 179881 | aaacggcagt cagagctccc tcagaagcct atctaagggt tcccggcaga aggcgccatc |
| 179941 | ttccgaggct ccggttgcac gaccatttgg agtttgatgg cctgaaggca agaggagaaa |
| 180001 | aaccaggtta ttagaaaaca tgtgttaaaa cgaaacaagg gagggtaagg acagcttaaa |
| 180061 | atccctaggc cttttaccag tttgcacagg gagagggaaa ccaaaagccc gactggtaaa |
| 180121 | aaaaacttta cccttttgcc agcatgtcat gcttgtgggt tcccttcctc tgagcccaat |
| 180181 | cctaagccaa ccagctgaag gtttgggaaa ttaactcttt ccagtttgga ggatgcatct |
| 180241 | gaggggagtg tcctatagta caaagacaca attacctatt agtgaagaga agacagtgga |
| 180301 | gaagaaagga aaaaaaggtg ctttttaaag gagtcccagg ggttcagcat gcattcaaaa |
| 180361 | ggggtacaga gtgaagatga atggctaccc atctagaaag aggggagcag gcatccctgt |
| 180421 | ctcctatctc tttctagcag ataccagggg tacatgaggg agagaaggaa gagcgtcctc |
| 180481 | tttccctctt ctgtccttgc atacccaagt cctggtgacc ttggcaggtg ctgccatggg |
| 180541 | tgccacagca gcttgcaccc atgaagcagg gagtacctag agaataggaa ttatcagcca |
| 180601 | gactcagtgg ctcacgcctg tacaacccag caccttggga ggccgaggtg ggcagatcat |
| 180661 | gatttcagga gtttgagacc agcctggcca acatggtgaa accccgtctc tactaaaaat |
| 180721 | acaaaactta gctgggtgtg gtggtgcatg cctgtagtcc cagctactcg ggaggctgag |
| 180781 | gcaggagaat cacttggtcc caggaggcaa aggttgcagt gagccaagat cacaccactg |
| 180841 | cactccagcc tgggcaacag agtaagactc catctcgggg aaaaaaaaag agagaatagg |
| 180901 | aattatccgc tcttacctat gtttctatac cccctacagt cagtagtctt ggagtttcct |
| 180961 | agacctcatt tatgctgtgg ggaaaagcaa gagagatcag attgttactc tgtctgtgta |
| 181021 | gaaagaagta gacataggag actccatttt gttatgtact aagaaaaatt cttctgcctt |
| 181081 | gagattctgt taatctataa ccttaccccc aaccctgtgc tctctgaaac atgtgctgtg |
| 181141 | tcaactcaga gttgaatgga ttaagggcgg tgcaagatgt gctttgttaa acagatgctt |
| 181201 | gagggcagca tgctccttaa gagtcatcac cactccctaa tctcaagtac ccagggacac |
| 181261 | aaaaactgcg gaaggccgca gggacctctg cctaggaaaa ccagatattg tccaaggttt |
| 181321 | ctccccatgt gatagtctga aatatggcct cgtgggaagg gaaagacctg accgtccccc |
| 181381 | agcccgacac ccgtaaaggg tctgtgctga ggaggattag taaaagagga aggaatgcct |
| 181441 | cttgcagttg agacaagagg aaggcatctg tctcctgcct gtccctgggc aatggaatgt |
| 181501 | ctcggtataa aacccgattg tatgctccat ctactgagat agggaaaaac cgccttaggg |
| 181561 | ctggaggtgg gacctgcggg cagcaatact gctttgtaaa gcattgagat gtttatgtgt |
| 181621 | atgcatatct aaaagcacag cacttaatcc tttacattgt ctatgatgca aagacctttg |
| 181681 | ttcacgtgtt tgtctgctga ccctctcccc acaattgtct tgtgaccctg acacatcccc |
| 181741 | ctctttgaga aacacccaca gatgatcaat aaatactaag ggaactcaga ggctggcggg |
| 181801 | atcctccata tgctgaacgc tggttcccag ggtcccctta tttctttctc tatactttgt |
| 181861 | ctctgtgtct ttttcttttc caaatctctc gtcccacctt acgagaaaca cccacaggtg |
| 181921 | tgtaggggca acccacccct acatatgcca tggatactag catgaccttt atccatgaaa |
| 181981 | tgggaggctt ggcttgattg gcaggaatta gccatgctca cttgcacgca ctgtgccttt |
| 182041 | tttttttttt tttttttttt tttttttgag acagagtctc gctctgttgc caggctgaag |
| 182101 | tgcagtggag tgaactcggc tcactgcaac ctccgactcc ctggttcaag tgattctcct |
| 182161 | gcctcagcct cctgagtagc tgggattaca ggcacgcacc accatgccca gctaattttt |
| 182221 | gtattttttt tttcgagatg gagtcttgct ctgtcgccca gtctggagtg cagtggcatg |
| 182281 | atctcagctc actgcaacct ccacctccca gttcaagcga ttctcctgcc tctacctccc |
| 182341 | aagtagctag gattacaggt gcatgccacc atgcctggct aatttttgta tatttagtag |
| 182401 | agacggggtt tcaccgtgtt tgccaggatg gtcccgatct cctgacctca tgatctgccc |
| 182461 | gccttggcct cccaaagtgc tgggattaca ggtgtgagcc actgtgcccg gccttgcact |
| 182521 | gtgcctttta acccctgtta tcatctgcct ctgaatccct tagatccagt tttctttcct |
| 182581 | ggggctttga ctcaaagctt ggaatagaat ttgaaacaaa aatatgtgtc taggaagggc |
| 182641 | tgcatggact ccttatcata agccaaatgc taaggaaggt gaagctgcag aattgagtcc |
| 182701 | tcctccaaca agggagagaa aaagatgtct tgtgacatgc ccagataatt ggtggctata |
| 182761 | gttatgtaag ctaggatttg ggtgcatggt gcttggcttt ggttagctct tactttccaa |
| 182821 | aaaaggaaac ctccgattaa tgggcatcct atttattccc atcacctggc aggatttgca |
| 182881 | ggataattgc tcagaactaa aatattgatc cagattttta cattacccat cctctccttc |
| 182941 | tttctgagct gcagctggag attgctggtt ggttcacagg aacaaggagt gttagcctaa |
| 183001 | aaatgtaggc aaaaacttaa acactaatga ctttagaatt tattgacaaa tgtatggtgt |
| 183061 | tttgaaacat aatttctctc tctccagtcc tcatttttgt taaaaaagca aattatggta |
| 183121 | ggattgagtt gtttgcaaaa tagactttag tcatatactt ggcctgatta tttgcataaa |
| 183181 | gtgcagcaag aataactatt tctacataag tcttttagat tggctttgat ggaactctgt |
| 183241 | tcttcaagga atttcagaca aaacgtttta aaactgagcc cagccatgga tttgtatcct |
| 183301 | caaatacctg taagttggat gatcttctcg tgttaaggtc ccatgacaaa tttggagctc |
| 183361 | ctagacctgt tagaaagtga cattcttggc caggcgtggt ggctcacacc tgtaatccta |
| 183421 | gcaccttggg aggccgaggt gggcggatca ccggaggtca ggagttcgag accagcctgg |
| 183481 | ccaacatggt gaaaccctgt ctctacttaa aatacaaaaa attagctggg tgtggtggca |
| 183541 | agtgcctgta gtcccagcta tttgggaaac tgaggcagga gaatcacttg aacctgggag |
| 183601 | gcggaggttg cagtgagccg agatcatgcc actgcactcc agcttggcga cagagcaaga |
| 183661 | gtctgcctca aaaaaaaaaa aaaaagaata ctcacggata atttccaaat tctggagaag |
| 183721 | ccaggcagag agagagaaaa atatgcttca aattttgttc acaggagtgt agcttactct |
| 183781 | agtattaaag gccttaaaga gttcaaaata attttccttg actctgagaa acaaaacaag |
| 183841 | gatcagcaat attccaagca aaagtcaaaa aggttgcttc agctttctga gttcagtcca |
| 183901 | ttcagttctt gttatgcttg atattcgtga acattttagc tcttcatgag tcctgtacat |
| 183961 | tttcctttat tccaatatca cagtctccaa agttatcaga aacctgtatt tgagagcacc |
| 184021 | tgtcagagtc ctatagctta ttacaaacca actttatttt tttttgagac aggatcttgc |
| 184081 | tctgttgccc aagctggaat gcagtggtga gatcttggct cactgcaacc tccacctcct |
| 184141 | ggattcaagc gattctcctg ccccaacatc ctgagtagct gggattacag gtatgtgcca |
| 184201 | ccatgcccgg ctaatttttg tatttttttt tttttagtag agatggggtt ttaccatgtt |
| 184261 | gaccaggttg gttttgaact cctgacctca ggtgataccc ctgacttggc cttccaaagt |
| 184321 | gctgggatta aaggtgtgag ccactgcacc cagccggatc aggcactttt aacttgaaaa |
| 184381 | catgaagttc tctttcctaa cttttttctt tctttctttt tttttttttt ttccgagaca |
| 184441 | ggatctcact ctgtcaccca ggctggagtg cagggatgcc atcacagctc actgcagccc |
| 184501 | tgacctcctg ggctcaagca atcctcccag ctcatcctcc caaatggctg ggactatagg |
| 184561 | cttagaccac catacccaac taatttttgt atttttgtgt gaagatgagg tctccctatg |
| 184621 | tttcccaggc tggtcttgat ctcctgggct caagcaatcc atcagccttg gcctcccaaa |
| 184681 | gcactgggat tataggcatg agccaccaag gacggccccc taatgttttt tatattggct |
| 184741 | aatgagcttt aagtttatac atacaaggca taaagaaata actttaaagt tatgttatta |
| 184801 | gatgtacagt gatatgattt agctctgtgt ccccacccaa gtctcatctt gtagctccca |
| 184861 | taactcccat gtgttgtgaa agggacctgg tgggagatga ttgaatcgtg gaggtgggtc |
| 184921 | tttcccgtgc tgttctcatg ataatgaatg ggtctcacaa gatctgatgg ttttaaaaac |
| 184981 | gggagtttct ctgcacaagc tctctctttg cctggtgcca tccacataag atgtgacttg |
| 185041 | cttctccttg ccttccacca tgattgtaag gtctccccag ccatgtggaa ctgtaaatcc |
| 185101 | aataaacctc tttcgtaaat tgcccagtct tgcatatgtc tttatcagca gcgtgaaaac |
| 185161 | agatgaatac agtaaatttg tactgggagt ggggcattgc tgaaaagata cccaaaaatg |
| 185221 | tggaagcgac tttggaactg gggaacaggc agaggttgga acagtttgga gcgctcagat |
| 185281 | gaagacagga aaatgtggga aagtctggag cttcctagag acttgttgaa gggccttgac |
| 185341 | caaaagcctg atagtgatat ggacaataag gtccaggctg aggtggtctc agatgaagat |
| 185401 | gaggaacttg ttgggaactg gagcaaaggt aactcttgta tgttttagca aagagactgg |
| 185461 | cagcattttg tccctgccct agagatttgc agaactttga actcgagaga gatggtttag |
| 185521 | gctatctggt ggaagaaatt tgtttattat tattattatt attattatta ttattattat |
| 185581 | tttgagatag agtctcactc tgtcacccag gccagaggac agtagcgtga ccttggctca |
| 185641 | ctgcaacctt cgcctcctga gttcaagcgt ttctcatgtc tcggcctccc aagcagctga |
| 185701 | gataacaggc atgtgcctcc atgcctggct aatttttgta ttttagaaga gacagggatt |
| 185761 | caccacattg gccaggctgg tctcaaactc ctgacctcaa atgatccaac tcagcctcct |
| 185821 | gaagtgttgg aattacaggc atgagccacc acacctggct gcggaagaaa tttctaagct |
| 185881 | gcaaagcatt caagaggtga cttgggtgtg gttaaaggaa ttcagtttta taagggaagc |
| 185941 | agagaataaa ggttcagaaa atttgcagcc tgacaatgcg atagaaaaga aaagccaatt |
| 186001 | ttctgaggag aaattcaagc cagctgcaga aatgtgcata agtaacaaga ggaacattaa |
| 186061 | cccccaatac aatggggaaa atgtctccag ggcatgtcag aggtcttctt ggcagcccct |
| 186121 | cccatcacag gcccagaggc ctaggagaaa tggttccgtg ggccaggtcc agggtccccg |
| 186181 | tgctgtatgt agcctaggga cttggtgcct tgtgtcccag ctgctccagc catggctgaa |
| 186241 | aggggccaac atagagctta ggccatggct gcagagggtg aaagccccag tccttggcag |
| 186301 | cttccacgtg gtattgagct tgcaagtgca cagaagtcaa gaattggggt ttgggaacct |
| 186361 | ccacctagat ttcagaagat gtatggaaat gcctggatgc ccaggcagaa gtttgctgca |
| 186421 | ggggtggggt gctcatggag aacctctgct agggcagggc ggaagggaaa tgtggggttg |
| 186481 | gagctcccac acagagtcct tactggggca ctgcctaggg cagctgtgag aagagggcca |
| 186541 | ttgtcctcta gccccaagaa tggtagatcc actgacagct tgcactgttt gcctggaaaa |
| 186601 | gctgcagaca ctcaatgcca gcctgtgaaa gcagccggga gggaggctgt acactgaaaa |
| 186661 | gccacagggg cggagctgcc agagaccatg ggaacccacc tcttacatta gtgtgacctg |
| 186721 | atatgagaga tggagtcaaa ggagatcatt ttggagcttt aagatttgac tgccctgctg |
| 186781 | gattttggac ttgcaggggc ctgtagcccc tctgttttgg ctaatttctc ccattttgga |
| 186841 | gtggctgtat ttacccaata cctgtactcc cattgtatcc aggaagtaac taacttgctt |
| 186901 | ttgattttac aggctgatag gcagaagtgt cttgcttttt ctttgctgag actttggact |
| 186961 | gtggactttt gagttaattc tgaaatgagt tgagactttg ggggactgtt gggaaggcat |
| 187021 | gattggtttt gaaatgtgaa gatatgagat ttgggagggg acagggatgg aatgatatgg |
| 187081 | cttggctgtg tccccaccca aatctcatct tgtagctccc ataattcact catgttgtgg |
| 187141 | gagggacctg gtgggagatg attgaatcat gggggtgggt ctttcccatg ttcttgtgag |
| 187201 | agtgaatggg tctcacgaga tctgatggtt tttaaaacag gagtttctct gcacaagctc |
| 187261 | tctctttgcc tgctgtcatc cacataagat gtgacttgct cctccttgcc ttctgccatt |
| 187321 | attgtgaggt ctccccagcc atgtggaact gtaagtccaa tgaacctctt tcttttgtaa |
| 187381 | attgctctat ctcgggtatg tctttatcat tcctgaaaat ggactaatac atacggcaat |
| 187441 | actcaagaga gcaagtgtgg atagcttggg ttcatgaggt ttttggtttt tgtcaaaagc |
| 187501 | ataaatgtat actgaaacta cccagaaaaa gcaaagtata aaacagtatg ctaccatttg |
| 187561 | tgcccataaa tgggatatgt gtagtgtaaa tgcatagaat ttgaccagat gtatacccag |
| 187621 | agaccagaac actcagtggc ctcatgttca tactttggca aacacatgta tagtatcctt |
| 187681 | aacttaaatg taccattgtt gtgtgcattc tagtgttatt gacatttaca tatctattat |
| 187741 | caagtcagat aaatcattct gtgtctttgt attttcactt cctattttgt atatttatgt |
| 187801 | atacatacac acataccaat acatatttaa ataaggtata acttcacctg gtccaaaaat |
| 187861 | caaaacaacg tagaaaggtt tacagtgaaa ggtcgcatcc ctgatgctgt cctcttcccc |
| 187921 | caggtgatca ccttattggt ttcttttcat acctttcagc attttctcct gcaagcacag |
| 187981 | atattctaac tcctcctttt ttacacagaa ttttttttta catagcattc ttctgcacct |
| 188041 | tccttctccc acttcacaat gcacatggag atttttccgt atttgtacat caggagcttc |
| 188101 | ctctttcttt gttaccacat taaattccac tgggtagatg taccataatt taactgggtc |
| 188161 | cttattgaaa gacaattgag ctgtctccta gacaaagcct tgtgcacctt cccgaacaga |
| 188221 | gggtctaacc aagcaggcag gatggggtta taaagtaggt ggggaggtgg gagagactcc |
| 188281 | accctcccag gtgggctgag gatggaggta aggccctgca acaggacaga gggaaaagtg |
| 188341 | gggatgagac atgggaggcg agatagcgct cactgttctc gctcagcccc ctcctccatt |
| 188401 | tgccgctgac ctgttggcct cccccaacct ctgagcctgc ctctgcctag gtaatttccc |
| 188461 | aagacccagg aggggtgaag ggtgaggtgc gattgccccc acctccttgc ctcccgcagc |
| 188521 | atctgctcca ggaccatgaa caatagctga cagctccatg gcccttgctg tccccatctc |
| 188581 | agcttccctg ggcatctaaa cctcagttgc cRtggggtag gaggacaggc tgaggaagca |
| 188641 | gaagcctgag gctgtctaga gtctcactcc tgcatcagca ggccaccacc tgtggttcct |
| 188701 | ccttgtgcaa atttgagagg aattgcataa aacactggag aaatccaaga ggggaagtcc |
| 188761 | acaagggcgg tggctccctc catgatctca gagcaagctg gtgtcagaac ctggacctag |
| 188821 | agcactgttg ggtgggaagt aggaggagag tcctgcctcc aggccagccg gggaagggct |
| 188881 | ccctctcacc tctacacgca gcgcatttct tggctcagct gccctgtagg ggatgcaggg |
| 188941 | tggggacagc agagatctgg gcctgggagg gagacagtac acaatcacat ggctgttgcc |
| 189001 | cctccctcag gccttgtcta cctctgactg tggctctctg gcaggaatag atggacatgg |
| 189061 | cctggcagat gatgcagctg ctccttctgg ctttggtgac tgctgcgggg agtgcccagc |
| 189121 | ccaggagtgc gcgggccagg acagacctgc tcaatgtctg catgaacgcc aagtaccaca |
| 189181 | agacacagcc cagccccgag gacgagctgt atggccaggt gaggataaag tcgggtctgg |
| 189241 | ggcaggggag agagctgcct ttgacagaga aaagttagca gggtgaagtc agctgtggaa |
| 189301 | ggtgatgaag gcagaggatg actgctgtga tcctggaggg cctcccctgg gaggacacca |
| 189361 | aatctacaat aataaaggaa ccacatacct aacacccact atgagcctat gagccttgtg |
| 189421 | ctttcactca tgccatgtta gtgaatatgc ccaaagggca gggtgggtgc gtggtagtct |
| 189481 | ctacttacgc aaatgaggaa ataggcccag agagggcgca ggtgcaagtg ggaaatgagg |
| 189541 | agagggactg tccatacctt tcccagcctt aattgtgtgc cagcccacag agctcaatgg |
| 189601 | cacagtagaa gaggagctgg gcccatcatg gtcatggtgc agcctacatg tacaaacagt |
| 189661 | ccaggtttcc acccccatta ggtactgaag agacaacgac caatccaata tgctggggag |
| 189721 | acttcccagg caagagacca ttggccttga cctgggaaga tgagtaagag ttttctagga |
| 189781 | ggaaaaagcg ggaaagggca gcctggtaga ggacagcatg cacacaggtc agagggtgat |
| 189841 | ggcacgagca atggcgggtc cagtgtggcc agagccagcg gtgccactgc ccacaaggaa |
| 189901 | ggggagggga ggccagggcc ctcatgccac aggtaaggcc actgaggcag ctcggggagt |
| 189961 | atgagctcca atttgaattc caagctcagg agagtgcctg tatttcattt ctctggtctc |
| 190021 | ctggcctgct ccctagaagg gttcacattc ccagagggtg aggatgtgcc cagggagaga |
| 190081 | ctgtggcatg gacaggctcc gagggctaaa gcaaagagag gacagcctgg aagccctgtg |
| 190141 | acatctgagt cattcccaac attccatttg cttattttaa aatcagggta aaatttaaaa |
| 190201 | taaataaata aaataagttc tccattttag ccatttttaa ctgtacaggt cagtggcatt |
| 190261 | aagtatgctc atgtatgccg ggtgcagtgg ctcacgcctg taatcccagc actttgggag |
| 190321 | gccgaggtgg gcggatcacc tgaggtcagg agtttgagac cagcctgacc aacatggaga |
| 190381 | aaccctgtct ctactaaaaa tacaaaaaaa aaaaaaatta gccgggcatg gtggcccatg |
| 190441 | cctgtaatcc cagctactcg ggaggctgag acgggagaat tgcttgaacc tgggaggcag |
| 190501 | aggttgcagt gagccgagat cgcgccattg cactccagcc tgggcaacaa gagcaaaact |
| 190561 | ctgtcaaaaa aaaaaaaaaa gtatgctcat gtggttgtgc aaccatcacc accatccatc |
| 190621 | tcctgacctc tttcattctc caaaactgaa atggaaactt tgtgcccacc acttcatttg |
| 190681 | cttttcagaa ccttctaaag cacctactct ttgccaagaa atggtgcaga tgtagacttt |
| 190741 | gagagataca gatgattatc attctcaggg ccatgagcta tatgagagtg atgatatttg |
| 190801 | ttgggccact tctatagcca gggagtactt ttcattatat tcagtaactc tttcagaggc |
| 190861 | agaaacccct gaccctgact ggcaaaaccc atgtctgcca aaccccaaga cccatgatgt |
| 190921 | gcaaggggtc ttgcaggaag accaagagtt gggacagcca aggaaaagca agtgtgaagt |
| 190981 | tgtgctggcg gggaagcatg ttctgtgtcg gccggcactg ggcgtggtgg cccagtgggt |
| 191041 | aggtctggct ccactcccat ggatttccct attgtttctc ctggttgctc agacctgtca |
| 191101 | cgcctctgcc atcacttgac cctaggtgca agggttcagc ccagaaatKt tatgcaattg |
| 191161 | actcatgatt tctcaggttt tctgcatcct ggcctagagt gatttccaaa gaaaaacctc |
| 191221 | caccatttct gcttgtcttg cctgcattgt atttaccttt ctaggattgc cttttccaca |
| 191281 | tttagtcaag cccgggttca ggcccacgtt caggctacag ctcctctgtt cccccaccac |
| 191341 | tctcaggatc |

Following is a NUMA1 region genomic nucleotide sequence that includes the NUMA1 SNP “NUMA1-AA” (SEQ ID NO: 4952).

TCTCTGCCCGACTACAGTCTACTAAACCTGAAGCCCACATGCCCTTCAGT
CCACATCCGTAGTGGCCTTTCCCTGACCAGCTCCTGCCCTATCTTTGCCT
TCTTTGAGACCAGACACAAAGTGTCACACCCTCTTTGCATGCACAATCTC
CCGTAGGAGGAAGGGAACCAATCCTCAGGGAACACCCAGGTGCAGAGCCT
[-/T]ACTCTAAGGCACTTGGCATGTGAGCTCTCTTAGGACAGGGGAACT
TGTTTCTCCTCAAAGCCACTCTGAGAAGTCACTTCACCCTCTTTTCAGAG
GAGGAAACTGAGTTCAGAGATTTAAGCAAATTGACCAGAGTAACAGTGAC
TAAATGGCAGAGTAAGATTTGGTCCACCTAACTCCAAAAGCCCATGGGGG
CTTAT

Following is a NUMA1 region genomic nucleotide sequence that includes the NUMA1 SNP “NUMA1-AB” (SEQ ID NO: 4953).

AAACCAATCACAGCCATCACACCTCTAATTCAGAACTACAAATCAACATT
TAGGGTCACAGCCCAGTACCCGGGAAGCCGCCAATTATGGGATAAGGAAA
GGGGCAAAATATGCAGGTCTCTGGAGAGACTGAAGCATAAGACAGGTGCT
GGGACATTCTTCTCAACCCTCTCTTGGGGAGCCTTCCAGACCACCTCAGC
CCTGAGGGGCCTCCATGCACACACCACCTTAACACCCACCTTAAAGGAAA
GGTCTCCATTCTCCTCCGAAAGCTGGTTGATTTTGCGATCCATCTGGCTC
TTCTCTGTCTTCAGGTCCTGGCACTGCTTCAGGGTTTCATGCAGCCGCAT
GGTAAGGCTGCGAGGGAGGGAAGCAGTGAGAAGAGAGTATGGG[T/C]TC
CTGGCTGGGTGAGGGGAGGGGCTGGGGCCTGGGAAGGAATGCAGGGAGAG
CTGTACCTCTCATTCTTGTCACGCAGCTCCTCAAGCTCCTTGGGCTCCAG
TGGGCTGGCCGCCTGCTTCTCATTCAGCAGGGCTAGGCGGTCAATGCGCT
GCTGCATCATGGCTATCTGTGCATCTGCCCAGAGTGGAGGGAGGAGGAGA
GGAGAATCAGAACTATGTGCATGAGCGGAACTATGTGCACAGAACTATGT
GCACAAGGGGAAGGGAGGAGACGGCATAGGGAAGAGGAGGAATATGGTAT
CCAATCTAAGGGAAAGAGAAGATGGGGACAAAGACAGAACCTAGCTTCCA
GGAGG

Following is a NUMA1 region genomic nucleotide sequence that includes the NUMA1 SNP “NUMA1-AC” (SEQ ID NO: 4954).

AGACGGCATAGGGAAGAGGAGGAATATGGTATCCAATCTAAGGGAAAGAG
AAGATGGGGACAAAGACAGAACCTAGCTTCCAGGAGGTCAGCCTGACCAC
AGGCCCAGGGCTGAGCTCTCAGCTCCAGGGGCCAGCAGAGGGGACAACAT
GGGAGGCCAGGGGACATCCTTCCGGCAAGTCTATTCACACATCTGCCAGT
GTGCCAGCCACCTACCCTTCTCGGTGAGGAGCTTGCGGTTCTCAGCTAGC
TCCAGCTCCAGCTCATCCCTATTACTTCTCTCATCAGCAAGCTGCTTCTT
CAGCCGTCTCATCTGGAACTGTGGGGTCTGCAGGATATCACCCATGGGAG
AAGCTGGAGAACCTGAGAGAAAGCTGAGGAGGGAGAAGGCCCATATGCAT
TGGTAAGCGCCTAAGAT[C/G]TGAAGCAATCAGCAACAGGCAGCAGTAA
GGGAGCGGGGTCTATGGAAGTGCGCACTAGCAGCTGGGAGGCCTGTGCAC
CTGGGTTGTTTGCAGTGCTACCTCAAGCCTCATGACATGATCTTGGGAAA
ACTTTTCTTCTCACTGCCTTAATTTCTTTGTCAGAAAAAAAAAAAGTGAT
ATATTCTCCTTGGCTCTGCAGAGATGTGCTAAGAGAAGAGACTTCATTGT
AGTCTCCGAGGAAGAAGGGTATTCCATAAACAGGGAACAGCTTTTGAGGG
AGGCCTTCTGCACATGACAAATCCCAACCACTTCC

Following is a NUMA1 region genomic nucleotide sequence that includes the NUMA1 SNP “NUMA1-AD” (SEQ ID NO: 4955).

GCCTGCTACCACATCCAGCTAATTTTTTTTTTTGAGACGGAGTCTCGCTC
TGTCACCCAGGCTGGAGTGCAGTGGCACGATCTCGGCTCACTGCAAACTC
CGCCTTCCAGGTTCACGCCATTCTCCTGCCTCAGCCTCCCAAGTAGCTGG
GACTATAGGCACCTACTACCACGCCTGGCTAATTTTTTGTAT[T/G]TTT
AGTAGAGACGGGGTTTCACCATGTTAGCCAGGATGGTCTCCATCTCCTGA
CCTTGTGATCCACCCACCTCGGCTTCCCCCAGCTAATTTTTTATATTTTA
GTAGAGACAAGGTTTCGCCATGTTGGCCAGGCTGGTCTTGAACTCCTGAC
CTCAGGTGATCCGTCTGCTTTGGCCTCCCAAAGTGCTGGGATTAGAAGC
GTGAGCCACCACGCCCAGCCTTTTTTTGTATTTTTAGTAGAGATGGGGTT
TCGCCATGTTGGCCAGGCTGGTCTCAAACTCCTGACCTCAGGTGATCTGC
CCACCTCAGCCTCCCAAAGTGCTAGGATTACAGGATAAGCCACTGCACCT
GGCCAGCTCTAGTCTTATTTTGCTGAAAAAGGGAGGCAATTGAGGGAAAG
GCCTGCTGGCCTGGGACACTGGAGACGTGGGTTTACCACACAGCCTTGGG
AAATTTATCTAACTCTCTGGGCCCCTTTGTACTTTTCAGCTTAGAGATTT
GGGGGTTAAAGTAGATCAGTAATTTCCAAACTCTTCACAGAGTACCAGGG
TCTGTAGAGATGCCTCACAGGCCTC

Following is a NUMA1 region genomic nucleotide sequence that includes the NUMA1 SNP “NUMA1-AE” (SEQ ID NO: 4956).

CACACAGCCTTGGGAAATTTATCTAACTCTCTGGGCCCCTTTGTACTTTT
CAGCTTAGAGATTTGGGGGTTAAAGTAGATCAGTAATTTCCAAACTCTTC
ACAGAGTACCAGGGTCTGTAGAGATGCCTCACAGGCCTCCATGACAGGCC
AAGAAGATGGCCTATGATCTCTGAAACCAGCCCCCACTTCAATCAGATCA
AAGTAATTCCATTCTGTTTTATATGCTGGGATTCTGCTTAAGATTTCATT
TGATAAAAAGAATCTTCTGCTTAAAAACATTTGCTTGCTGACTCCATATT
AGGTATTCCCAGAAGAGCCTGGTCTGCTTTCTCTCTCCACCCCCCAACCC
GCCATGTCTCCCTTCACCCCCAGGGGCTGTTGGTAATTCCA[T/C]TGAG
TCTGAGGCAGGGCCCCCAAGAATCAAAGCTGCCTCAGATTCAGGTCTGTT
CAGCTTTGACAGTGTTCCCATCATCACTGGACAAGGGACAAGAACAGTCT
CATCCAGTCTGAGGTTAGCCCAA

Following is a NUMA1 region genomic nucleotide sequence that includes the NUMA1 SNP “NUMA1-AF” (SEQ ID NO: 4957).

TCCAAGGAAAAGCAAGTGTGAAGTCGGGCTGGCAGGGAAGCATGTTCT
GTGTCAGCCGGCACTGGGCGTGGGCCAGGGTGTGGGAGGTGGGTAGG
TCTGGCTCCCCTCCCATGGATTTCCCTATTGTTTCTCCTGGGTGCTCAGG
CCTGTCACGCCTCTGCCATCACTTGACCCTAGGTGCAAGGGTTCAGCCCA
GAAAT[G/T]TTATGCAATTGACTCATGATTTCTCAGGTTTTCTGCATCC
TGGCCTAGAGTGACTTCCCAAGAAAAACCTCCACCATTTCTGCTTGTCTT
GCCTGCCTTGTATTTACCTTTCTAGGATTGCCTTTTCCACATTTAGTCAA
GTCTAGGTTCAGACCCACGTGCAGGCTATAGCTCCTTCGTTCTCCACCA
CTCTCAGGATC

Following is a GALE genomic nucleotide sequence (SEQ ID NO: 5).

| >1:23536001-23622950 | |
|---|---|
| 1 | ggcaacctag tgagaccttt tctctacaaa aaataaaaat aaaaaaatta gctgggcatg |
| 61 | gtggcataca cctgtagtcc caagctgagg caacaggatt acttgagccc aggaggtcaa |
| 121 | gactgacagt gagccgtgaa tgtgtcaggg cactccagcc cgggcaacag agtgagaccc |
| 181 | tgtctcaaaa aaaaataaag taagggctgg gtgcRgtggc tcatgcctgt aatcccagca |
| 241 | ctttgggagg ccgagacggg cggatcacga ggtcagaaga tcgagaccag cctggctaac |
| 301 | acggtgaaac cccatctcta ttaaaaatac aaaaaaaatt agccgggtgt agtggcgggc |
| 361 | gcctgtagtc ccagctactc aggaSgctga ggcaggagaa cggcatgaac ccgggaggca |
| 421 | gagcttgcag tgagccgaga tcgcgtcacc actccagcct gggcgactga acgagactat |
| 481 | actgagtaga ctatacttgc tgcaaaaatt caaaaaagtg tacggagcag atgagagcct |
| 541 | ccttgcccat cctcctcccc tagtatagcc ttgaattcat ctcccaaaca gaataggatg |
| 601 | ggtcttttag ttactctcgc agagggctct gtagtgctga taagagagct gaggagcccc |
| 661 | tcttaggttt tcagatgaca cgtcaggcta gccttatatt taataattta aatttaataa |
| 721 | taattatttc agttttcaag aaacttactg aatctcacct atgtgctaaa tgttgctgat |
| 781 | tgaagatggg taaatagcct ctgaccttag ggatctcatt cctgtgttga acactcgtgt |
| 841 | gtaaaccaag ggttacaatg gtaatccccc agtgaataca tacagagtac caattttaga |
| 901 | agtggttgaa aagttttttt aaaaaatcag agaattacac atgtataatt agctgctcat |
| 961 | cctagaagtc atatatccag accaggcgtg gtggctcatg cctgtaatcg cagcactttg |
| 1021 | ggaggctgag gcgggtggat cacttgaggt caacagttcg agaccagcct ggccaatatg |
| 1081 | gtgaaacccc gtctctacta gaaatacaaa aattagccga gcgtagtggc gcatgcctgt |
| 1141 | agtctcagct actcgggagg ctgaggcaga agaattgctt gaacctggga ggcagaggtt |
| 1201 | gcagtgagcc cagatctcgc cattgcactc cagcctgggt aacagagtga gactctgtct |
| 1261 | caaaaaaaaa aaaaaaaaaa agtcatatat ccaaatgtaa gagtactttg caggtaagcc |
| 1321 | tgactcagtc tgacataggg gtggctgtct gggagcagca cacccactcc aggatccccc |
| 1381 | agaagcattc gttgtttctt tctgtttttt tttggttttg ttttttctgt tgcccaggct |
| 1441 | ggagcagtgg tgcgatcatg gctcactgca gtcttgatct cctgggctca aatggttctc |
| 1501 | ctaactcagt ctcccaagta gctgggacca caggcacgca ccaccatgcc tggctaattt |
| 1561 | taaaattttt gtattttttg tagaggctgg gtcttactac gttacccagg ctggtctcaa |
| 1621 | actcctgggc tcaagcagtc ctcccgtctt gacccccaaa gtgctgggat tacaggcatg |
| 1681 | agccactgtg cccaacctca ttgtttcttt tcattttggt tattgagctc tctctggata |
| 1741 | acatctgcag aatgtttcat gtgtcccgag tatgtgccag gccaattact cagcaattct |
| 1801 | gcttcaagaa tcagtctgta ttccattaca ccagtggtac attttcaaaa tggaaggaat |
| 1861 | ataaaatgta aaagtatttg ctacgcagag gagggagaca ctactgctgg gactagaatc |
| 1921 | gaagttttct cacacccagt tcaatgcctt ttctacaaca tccctcagtt gagccttagg |
| 1981 | gtaccaagtg gatggtcatt agtcttgtat ggactttaYa tccaatgctg attataaggt |
| 2041 | ctagatgatg ctaggtggca gagattcaaa gttggacagg tcggttactg tacttgataa |
| 2101 | ataaacagcc cagtggcaag acagataaat aaactgacca tttacaccct gaaaaggtgg |
| 2161 | tcagttgctt agggaactgg gggaggggcg ccaaatggag gagtagggac atggtagaga |
| 2221 | gggcttttca gcatagtgag tgaatgaatg ctcatttgaa gaaacaactt gtgtttaaga |
| 2281 | tgtttttctt tcttccagag ctagaatctg gaagaatctc tgggctttcc tgctttgagg |
| 2341 | gagagatcag gctaggtgca ttgccaattt gctgctgaag atctgattag gttctgcttt |
| 2401 | gatgtgatca aatgagatgt aatcagtgct ggtactttaa gtgagtttgc aactatttag |
| 2461 | ttgctctttt tagtagtagt attagtaatg gtaaaactta gtgcttttag gatgagagta |
| 2521 | gacggcctga agaataatat cttgggctct tctaatcaag ccatacggct cattggcaag |
| 2581 | tcatcagagg aaacatctat ctatctatac attcatgcaa taagtattta tttagtgcct |
| 2641 | atgtgccaga cactgtttgt ggggaaaaca agacacatgg gctattattg cctcatggaa |
| 2701 | ggaactaagc tgtttggatt ttgaaaaatc cctttctctg ggcctcaatt cctcgacctg |
| 2761 | ttcagaaatg tgcgctcaca atggtcctag acatcttaga ttcaggttcc attttcagtt |
| 2821 | ctttcctgtc ctgcggccat ctgcaagtta atttggtctc cctgaacctc agtttcctca |
| 2881 | tctgtaaaat gaagatcata caactcgttt catcaggttg tactgaggct ggaaaagata |
| 2941 | gtctaactac tgacgcatcg tgtgctcagc ctagggtggc tgctaccgcg ggtgcctgcg |
| 3001 | gactcctagg acgggcacca gcccgggacc cctgacaagg tgttgattag ggtgtggcta |
| 3061 | agccccaggc gggagaaagc gttggcattt ataaggccag ggtctggcgc gStggcctca |
| 3121 | ttccgtcttg aagtagaccc tggagaggtc ttccaggctt gggctcagca cgcgagcaca |
| 3181 | gggagcgggg gtgccggcgg cggaggcgcg tacggcccga gcagccccca gcccccagcg |
| 3241 | cgcacacttg ggccaggggc gcgaggagtg cgcggccgcc gggtgggcgc gcccccagcg |
| 3301 | gccctggggg cggagcctgc agcccaccgc cccccacaca ccccagccga ggcccggaag |
| 3361 | gggccaggcg cgcttccgtc tccgccgggg atgcacggag ggcggagctg cggcccgagg |
| 3421 | acgcgacgcg agcccaRttc cggcgaggag gccgcgccag tgacagcgat ggcggcggag |
| 3481 | tcggcgctcc aagttgtgga gaagctgcag gcgcgcctgg ccgcgaaccc ggaccctaag |
| 3541 | aaggtaagcg agggggcggc gcgtagcggg atgggcgttg ggtcggtgag ggccccgtaa |
| 3601 | cggtcgcggc ccgagcgtgg cggggttcgg gggctgggac cctgagccag gccccgcggg |
| 3661 | gctggggtcg tgaagttcgc ggccattgac cgctgcccgc gcgttgggac cccgtgcgtc |
| 3721 | gggcgtcggg acgcggggcc ccggccgctc cccctccctc acgggcccgg gatgggggcg |
| 3781 | cgcccggtgc ggacaccgcg tgcctagcgc ttcctcccag aggcgctccg tggcgtcccg |
| 3841 | agccagctgt ttcctgcggg agagcgtgcc cggggcggga gggcgctggc cgccgcctcg |
| 3901 | ttggcctccc ggtgcctgcg ctggtgcccg gctccccgcg gagcggcgcg gactccccag |
| 3961 | cgggtgcgtg ttcgctggga ggagaagacg ggcgcccgaa cgctgcggag cctggactcg |
| 4021 | cggtctggtc cgcgcctctc ggagacgcgg ttactactct ccggctggat atcagagcgt |
| 4081 | ggacctttcg ctttctcagt gaatcccttt caaaacagaa taaagcctcg cgtcacctaa |
| 4141 | gcatactcct gaaaacttag gttggaattg aaaaagaggc gggggcccgg ggttggaagt |
| 4201 | cgcagctagg tttccttttg gccattaata ggaaggtaag agctaccgtt tctcgttgtt |
| 4261 | cgagtgatgg acaaccattt ttaattgtag ttaactagtt tttgttgttt attgtacaaa |
| 4321 | ctgaaYatta agttaaacgc gttcttgtac cttttcatct ttctgtcact cacttctctg |
| 4381 | gtctccagga gacagtctgg tggttgtcag gatttttttt tttctttttt ttttttgagg |
| 4441 | cgagtcttgc tctggtcgcc caggctggag tacagtggcg cgatctcggc tcactgcaac |
| 4501 | ctccgtctcc cgggttcagg caRttgtctg cctcagcctc ccgagtagct ggtattgtag |
| 4561 | gcgcccgcca ccacgcctgg ctaatttttg tatttttagt agagacaggg tttcaccatc |
| 4621 | ttggccaggc tggtcttgaa ctcaRYtgtc aggatttttt aagtggaagt acaacgatga |
| 4681 | aaaagcactt atgtgtttgg cattctccgt catcattctg gccggggcgg gcagttctag |
| 4741 | gagttggaac tcagtcctgg tggaaaagga agtcgtggag ggagggctag ggccgtgggg |
| 4801 | gaactgctct gctgagcctc ttcctcacct gctgcttcct aggactaacc tgaaaggcta |
| 4861 | aggtaccagg ctgaaagtca gtgctcagaa aaccaatcgt cattctttgg ggtttttttt |
| 4921 | cttgaagagc cactttctct ttaccttgtt ctagcctgtt ggaggtaggg tttctgcaat |
| 4981 | tccaaaggcc gtacacagcc tctcaccatc agaccacttt ttaaggctct tcgttcatac |
| 5041 | ctagctcgaa gattcacttc ctcaggaagc cattttagtt acaaatctgg gaaaacttaa |
| 5101 | aatgctttca ttgtgccatg ttttctgttg cagcttcagt accgtaccta gtggtcaggc |
| 5161 | atacttacaa gtttcttttt acagtaaccc cttgtggaca tctaataaat ggtcattatt |
| 5221 | ttttagtact agtttgtttt cctgaacact gtaagatctg tgactgacgt ttgatacctt |
| 5281 | aaagcagtgc catataataa ctacccacta tttgttcttt atttctgtca gataaaaatg |
| 5341 | ttctatgtag tgtctacagt catttttttt ttaactagaa tttagatttg gaagtagttt |
| 5401 | ttctattagt tgatttgcat gaaatacaaa attaggaaaa ggcttattcc acctcaacct |
| 5461 | agttgaacta ttaatgattt tttttttttt ttgaggattt gggctctttc tagatagaaa |
| 5521 | atcaccctga acttctagct ttgcattgtg aagtgagcat catgaagatg agaaaatgtt |
| 5581 | gggagatcat ttttgcaaag ggcataatag tcggcattca gatatgagtt aactgcagag |
| 5641 | ggaaaattgc aagctgtcat gttggccttg ttcctctcaa ccttctggta acctaacaag |
| 5701 | ctcctacagg ttgtatgtga aattgcaaga tgattatata gccctgttga atttacaacc |
| 5761 | agatcttgct ttcaaaccat tattagccaa gggtttgatt ccacacctgt gttcatggat |
| 5821 | tttttggtaY tagacattgc tgtaactctg ttttcacttt ttcatctgtt atcttggctc |
| 5881 | acttaaggga gaaggtatca gcagcctagg accacttggt ttctgttttt atgtttcata |
| 5941 | gttcatggct gataaaaatt acctgtcctt aggccgagtg cagtgcctca cacctgtaat |
| 6001 | cccagcactt tgggaggccg aggtgagtag atcacctgag atcaggagtt cgagaccagc |
| 6061 | ctggccaaca tggtgaaacc ccttctctac taaaagtacg aaaattagcc aggtgtggtg |
| 6121 | gcaggcgcct gtaatcccag ctgctcggga ggctgaggca ggagaacact cgaacccggg |
| 6181 | aggcagaggt tgcagcgagc tgagatcgcg ccattgcact ccagcctggg caacaagagc |
| 6241 | gaaactccgt ctcaaaaaaa aaaaaacaaa aaaaacacac acacacagtt ggccgggcgc |
| 6301 | agtggctcac gcttgtaatc acagcacttt gggaggccaa gaggggcgga tcatctgagg |
| 6361 | tcaagagttc gagaccagcc tggccaacat gatgaaaccc catctctact aaaaataaaa |
| 6421 | aaattagttg gtcatggtgg tggtgcctgt aatcccagct actcgggagg ctgagacagg |
| 6481 | agaatctctt gaacccagga ggcggaggtt gcagtgagcc gagatggcac cattgcactc |
| 6541 | cagcctggac aacaaKagca aaactccWtY tccaaaaaaa aaaaaaaaaa aaaaaaggat |
| 6601 | cctcgtcctg tgttacattt tcactgtgaa agctaccaag aacattttga tggcaggtga |
| 6661 | cattaaaaat attttttgct tttttgggtt gaaggattgt acaggggaca actgagatgc |
| 6721 | ctcaacatgt accttgcaaa tgtttgatat ttctcagttg cctgttacca gttcttacaa |
| 6781 | gctctatagt tggtatcttg tttgataaca gcaagtttcg atccacctga ggatgcccaa |
| 6841 | tgtgcctttg ccttgttatt gctttgttct ttcttgaaat ttactgaaaa tagaggggtt |
| 6901 | gtgaagtcct tttttctctt gtgttagact aaatgagtct gaaaatcttg agggttttag |
| 6961 | ttttgtctgc ttttaaatca cccctaataa cccaaagtaa agcagaaaga caggatttgt |
| 7021 | tcttccagtt agtgctaggc tttgagataa acacacccac agtgacttcc agcctcatcc |
| 7081 | ttatatgttg gctttgtaga ggtcctggga cacctttgtt cccttggtga cctcctttac |
| 7141 | tatgtgggtt ctgaactgtc taatgacatc acaggaggag tttttctgct ttcttgcaag |
| 7201 | tatgctaaga agtatatgtg ctgccgctct attttcattt cctaaggaca gtgttctgat |
| 7261 | gttggatgct tctcctggct ttacttaact gtagtagaca catttgactt tccagcttgt |
| 7321 | taagcaagtt acctaacctg attgggttca gttgcaatat ctgtcaaatg gggagaataa |
| 7381 | catccacttt atagagttgg ttagtgggaa tgaaatgagg taacgtgtaa gagcttagtg |
| 7441 | tagtagctga cacacaatag atggtatgtg aatattccca acctacccct tccaaggaat |
| 7501 | ggcattatga gagaaccact tgtttttgac cttttcagtg atttaaaatt ttcctttaag |
| 7561 | tgaaggaatt ggcattagtc tttgcaccca tagcttgggt ggcaagaaac acaaaatttt |
| 7621 | tcctgagttg gatgatggtg gtgataggat ttgaagtggg ggataatttt cactcatcta |
| 7681 | tgaaggtgag actccctaga tatgccactt ggcctcttac ttgatctcaa gattatcact |
| 7741 | tgtgatccaa ttgctctgtc tggtgaccat gttaagtaat gattgaagta gaatgaccag |
| 7801 | aaaaacagac ttgttactcc acctaatagc aatatggcat accaattata gtctgggcca |
| 7861 | cacatggtaa aagcagatct tgttatataa tatttaaatg caaaggtaac cacagttatc |
| 7921 | tattttaaac tacttgtatt ttgaagcact ccttgtatgg taagtatgtt tagatacacg |
| 7981 | tagacatttc ctagagaaag gtttggccag tcataacttt tgttagagca agtaggaatt |
| 8041 | ggctgaggaa tatctggttg aagatcaagg gcagaacgct gaaagctgtc agtcgatgaa |
| 8101 | tgaaatgata agtatttagt gagctttcat agtgttctag acactatgac taaggactgc |
| 8161 | acatggtttg tctctgaatc ctcatagttt gtgaagtaga gacaggacca agttcctatt |
| 8221 | ttgcagatga agtaactgaa gtttggggag gtttttgact tattcaagat cactcatcga |
| 8281 | gtgacaaagc cagattgtca agcctgtctc tgactccaga actccatctt aacagctctg |
| 8341 | ctgtactgat tttgacctag ttttaggaac tataggaagg aaatgtgatc aaacccagat |
| 8401 | cacttttcta atctacccag gcattttgtg tccaccgtta acatccaagc tctgattctg |
| 8461 | gcaacttgaa attactcttg gctttacact tatgtagaag aaaacaattc tgttaaacac |
| 8521 | tgtggccctt ctggaaacat gatggtcttt tcctgtagag gccttttctt ccttttactt |
| 8581 | cctctgtgcc aggtccatgg gtaattcccc taaaattggt acctttctca taaattctat |
| 8641 | caaaactagg gcaaaaatga tagttgttga tggcttttat cagagttggt aagttttgat |
| 8701 | tctcttctct acatcttatt tccccttggt gtagcacagt ggttgaaagc agggaatcta |
| 8761 | aagccacaga cctgagtgaa tcttgcccca cttgcatctt gggacaagaa acataacttt |
| 8821 | tatgagccta ttttcatact tgtaaaatag ggatattact tactactgta aataggcata |
| 8881 | atacttatac tcattactgt aaatggttag cactaagcac ttaataatca gatattcttg |
| 8941 | ttaaagtgaa tttgactatt gaaattaatg ttttcttctc tgcagctatt gaaatatttg |
| 9001 | aagaaactct ccaccctgcc tattacagta gacattcttg cggtaagaac tgtgtgactt |
| 9061 | taaatattat ataatgatat cccattccct tctcactgga gagtgtgtag aataaggtta |
| 9121 | caagtatggg ctttggaagt gaaagaaacc agacacaaaa ggccatatat tgtctgattc |
| 9181 | catttatatg aaatgtccag aataggcaaa tccatagaca gaaagtagat tagtggttgc |
| 9241 | caggggctag gagaattggg aggaacagga tttcttttta gggtaatgta aatgttctgg |
| 9301 | aattagatgg ttataatggt tatataatat tgtgaatgtt ctggaaacca Ytgaattatg |
| 9361 | tactttagtt aaagtggtga atattgttaa atgactttat ttcaaaatta gaattaaaag |
| 9421 | aacattgact ttggcatcaa ttagtcccag attcaaatct gagttctatg actttaggct |
| 9481 | gttcctatgt aaaatgggtc tgattcctgc ttcacagggt tattgtaaag actgattcag |
| 9541 | aactgtatgc aaagctctta tcttagtgcc taacacctgc cctaatcact cttgttatta |
| 9601 | aagttctgaa cttggtacgc tttgctactg tgtgtcatta gagatttggg gcatagacac |
| 9661 | aaagtaattt agtgttaaga tttgcagggt atgttgtagg aaggaagtat tgcttgttga |
| 9721 | gtttctcaaa gtagaaagcc ttatttcttt agaaaaacgt tagcctagtt ccagacccct |
| 9781 | ctaacaatta ctttgtcaaa ttttaggaga ctggggttgg gaaaacagta aatagcttgc |
| 9841 | gaaaacacga gcatgttgga agctttgcca gggacctagt ggcccagtgg aagaagctgg |
| 9901 | ttcctgtgga acggtaagaa catttctctt taggtttgtt caatttcatg gttaaattta |
| 9961 | cagatagaaa aggttttctt tattatttct cttttgcctt ctgaaattgt tttgcctcaa |
| 10021 | ttataaaaaa taatccatga tcattgtaga atattagaaa atattaaaaa tagaaatcaa |
| 10081 | aatttcctat ttctatcacg caaagacaaa cgttatgaac attttggtac gtttcttttt |
| 10141 | tttttttttt ttttttttga gacagagtct cgctctgttg cccaggctgg agtgcagtgg |
| 10201 | cgtgatctcg gctcactgca agctccgcct cccaggttca cgccattctc ctgcctcagc |
| 10261 | ctcctgagta gctgggacca caggcacccg ccaccacgcc cggctaattt tttgtatttt |
| 10321 | tagtagagac agggtttcgc cgtattagcc aggataggat ggttttgatc tcctgacctc |
| 10381 | gtgatctgcc cgcctcggcc tcccaaagtg ctgggattac aggcgtgagc caccgcggct |
| 10441 | ggcctggtat gtttctttct aactgctcct taaatggcat ttttttaagt tgtgaactag |
| 10501 | ataaatagtg agcagccccc attttatata acaaattaaa catgtcccca tgttttaata |
| 10561 | aatctccaca ttttcatgta tatgagatat ttgatctttg catcttgtgg ttaaactcac |
| 10621 | catttctcat ttgggttttc ccagttttaa gatttgagta tctttaagca taacctactt |
| 10681 | atcttcttct taagaccttg ttgtttctaa ctctgcatct tcacttcttt ggtgagttca |
| 10741 | gattctgtga ctttgtccct caagaagcaa gatttagacc tactttgtct gctttttctt |
| 10801 | ttattttaga aatgctgagc ctgatgaaca ggactttgag aagagcaatt cccgaaagcg |
| 10861 | ccctcgggat gccYtgcaga aggaggagga gatggagggg gactaccaag aaacctggaa |
| 10921 | agccaYgggg agccgatcct atagccctga ccacaggcag aagaaacata ggaaactctc |
| 10981 | ggagctcgag agacctcaca aagtgtctca cggtcatgag aggagagatg agagaaagag |
| 11041 | gtgtcacaga atgtcaccaa cttactcttc agaccctgag tcttctgatt atggccatgt |
| 11101 | tcaatcccct ccatcttgta ccagtcctca tcagatgtac gtcgaccact acagatccct |
| 11161 | ggaggaggac caggagccca ttgtttcaca ccagaagcct gggaaaggcc acagcaatgc |
| 11221 | ctttcaggac agactcgggg ccagccaaga acgacacctg ggtgaacccc atgggaaagg |
| 11281 | ggttgtgagt caaaacaagg agcacaaatc ttcccacaag gacaaacgcc ccgtggatgc |
| 11341 | caagagtgat gagaaggcct ctgtggtgag cagagagaaa tcacacaagg ccctctccaa |
| 11401 | agaggagaac cgaaggccac cctcagggga caatgcaagg gagaaaccgc cctctagtgg |
| 11461 | cRtaaagaaa gagaaggaca gagagggcag cagcctgaag aagaagtgtt tgcctccctc |
| 11521 | agaggccgct tcagacaacc acctgaaaaa gccaaagcac agagacccag agaaagccaa |
| 11581 | attggacaaa agcaagcaag gtctggacag ctttgacaca ggaaaaggag caggagacct |
| 11641 | gttgcccaag gtaaaagaga agggttctaa caacctaaag actccagaag ggaaagtcaa |
| 11701 | aactaatttg gatagaaagt cactgggctc cctccctaaa gttgaggaga cagatatgga |
| 11761 | ggatgaattc gagcagccaa ccatgtcttt tgaatcctac ctcagctatg accagccccg |
| 11821 | gaagaaaaag aaaaagattg tgaaaacttc agccacggca cttggagata aaggacttaa |
| 11881 | aaaaaatgac tctaaaagca ctggtaaaaa cttggactca gttcagaaat tacccaaggt |
| 11941 | gaacaaaacc aagtcagaga agccggctgg agctgattta gccaagctga gaaaggtaac |
| 12001 | cccttcagcc ccttggtggc ttcccaccca gagcacctgg ccctgcagag ctcagaggat |
| 12061 | tgccctgcca ctgttttcct tagcttgctt ttggggggca tgttgaattt cagtgtgcag |
| 12121 | taatttcctc tgggcttcac tggttgtcag ggtatatggt accgcaaata agaattttct |
| 12181 | gcattattct cttgtgatgg gctcatctgt atttatttcc tgactgaggt ggtgtcagac |
| 12241 | atgactgtgg ccccctccaa cctggatctt cctgtgcaag atgtccagtt aatgctcccg |
| 12301 | ggaatagact gtaggaaaga gagctgtgct tctactcact gactctccac ccatgggtct |
| 12361 | gtcccctgca ggtgcctgat gtgttgccag tgttgccaga cctcccgtta cccgYgatac |
| 12421 | aggccaatta ccgtccactg ccttccctcg agctgatatc ctccttccag ccaaagcgaa |
| 12481 | aaggtaattt tctatatctt ctttttctta atgggaaaaa gatttaaaaa ttcattcaag |
| 12541 | gcagggtaca gtggctcaca cctataattc cagcactttg ggaggccgag gtaagaggat |
| 12601 | tgcttgcggc caagggttct aaaccaacca ggcgtggtgg ctcactcctg taatcccagc |
| 12661 | actttggRag gccgaggcgg gcagatcacc ttaggtaaga agttcgagat tagcctggcc |
| 12721 | aacatggtga aaccctgtct ctactaaaaa tacaaaaatt agccaggcat ggtggtgcgt |
| 12781 | gcctgtaatc ccagctactc cggaggctga ggcaggagaa ttgcttgaac ctgggaggtg |
| 12841 | gaggttgcag tgggccgaga ttacgccatt gcactcaagc ctgggtgaca agagggaaac |
| 12901 | tctatttcaa aaaaaaggag ttctaaacca ccctggtcaa cacagcaaga tgctgtctct |
| 12961 | acaagagaaa aaaagtagcc aggcatggtg gcacatccca ggtactcggt aggctgaggt |
| 13021 | gggagtatca cttgagccca ggagttcaag gctgcagtga gctctgattg catcactgca |
| 13081 | ctctagcttg ggtgacagag caagactgtt tcaaaaacaa acaaaaaaac tatcaccata |
| 13141 | acatcagatt tttgatgcat ttcctaagtg catagactct gaagtttgat ggcttgggtt |
| 13201 | tgaatcccaa ctctggtttc tgcaagttat atgaccttga gaaatatctt aatttctttg |
| 13261 | gaccttagtt gtatatattg tgtaaatggg agttataatg gtatctgccc catagggctc |
| 13321 | agtttaatga attcagaagt gatgcctttt tgctctgggt ttatcacata gtaattgttc |
| 13381 | agtaaattgc tgctgctgtt attagcagcc ctttatccta tgtgttacat aggatttttg |
| 13441 | tctgaaagaa aaaaatgtct aattttcata gcaaaagaag tagaagccac ataagggtct |
| 13501 | atcagcagga gactaacaga taaatcatgg ttcatctaca cagtggcatc ctatgccaca |
| 13561 | gtaaaaaaaa aatgtggaaa gtctatgcac tgacatggaa tgatctctca gatgtggcaa |
| 13621 | atgaaaaaaa gcaatgtatg tagtgagtat gttattaatt gattcattgg cttaaaagga |
| 13681 | ataagataca tttgcttttt tttgttgttg ctgttgagag tcttgctgtg ttactcaggc |
| 13741 | tggagtgcag tagctccatc ttggctcact gcagcctcca cctcctaggt tcaagcaatt |
| 13801 | cctctgcctc agcctcctga gtagctggga ctacaggtgc atgttgggca ggctgatctc |
| 13861 | gaactcttga cctcagatga ttcatStgcc ttggcctcct aaagtgctgg gattacagga |
| 13921 | gcttattttt aaattctctg gaaggatcta caaaaatctg tagcgtggat tgccattggg |
| 13981 | aaagggaagt aggaggctga agggggtaga aggagagttt tcactggatc acttagggcc |
| 14041 | ttttgtgttt ttcttgccct atagcgttct cttcacccca ggaagaagaa gaagctggat |
| 14101 | ttactgggcg cagaatgaat tccaagatgc aggtgtattc tggttccaag tgtgcctatc |
| 14161 | tccctaaaat gatgaccttg caccagcaat gcatccgagt acttaaaaac aacatcgatt |
| 14221 | gtaagtcaca cgcttctctc tagctctcaa gcacattgta gcacttggac cttatcactg |
| 14281 | ggtggcttgg ctgctactca gtgtcttcac ctttggtttc tctgcagcaa tctttgaagt |
| 14341 | gggaggagtc ccatactctg ttcttgaacc cgttttggag aggtgtacac ctgatcagct |
| 14401 | gtatcgcata gaggaataca atcatgtgag tattctgttt gggtgggaag agggcagttt |
| 14461 | tctggcttgt acctccagaa atagctttgt ccctgtgact ccgctgtggc tgaagggcag |
| 14521 | atcagtggtc agagcattgt tctggatata tttggtttgc cactgggcct ttaaacaagc |
| 14581 | atgttattag cacttgctcc tggcacctta agtgggcctc ttgggctagg atcagggtgg |
| 14641 | gattcaaagg gagaagaaaa acacttaaca tctcccttga gcttgcctag tcatgaaggt |
| 14701 | aaagggagaa ttctggcagc agagtagctg ggacccaggg cggggaaagc actttgtgtt |
| 14761 | gacactctga catctggtga ttggctggat gtctcttaag aaacatcaag atttatccag |
| 14821 | aatcctcctt aggatgtagg gtatacacat ttgattgcat cagctgtctc tctttaattc |
| 14881 | tgaaagttct atgacctaac accttttttt ttttttttgg agatagggtc tacctctgtt |
| 14941 | gcttcgactg cagtacagag gcctaatcat ggcttactgc atccttgaac tcctgggctt |
| 15001 | aagtRatcct cccaagtagc tgggactaca ggcacatacc accacagctg gctaatattt |
| 15061 | ttatttttta gagagatgga ctctcagact cttggcctcc tgaagtgctg ggattacagg |
| 15121 | catgagccaa cgcgcctggc caataactta acatccttct taggttgccc ctttgccatc |
| 15181 | acagttcagc gtagtgttgg gaagtacctg atttgacttg atcgcagttc taccctttgc |
| 15241 | tgattttcta aaggaaaaga ctgatgttac acagattttt gtgatattct gtcacagcca |
| 15301 | ccaaaagcaa ctagcatagt gcctcacact tcgcattttt aaaatttagg gagttatttt |
| 15361 | tttcctagaa ttgtggtatt ttaacaWact actaagatga agaccaaaaa atttttggga |
| 15421 | aactggtcac tcctaggtaa ttaaataatc agaaaaagat atgaggctgg gcatggtgac |
| 15481 | acacgcctgt aatcccagca ctctgggagg ccaaggtggg cggattatct gaggtcagga |
| 15541 | gttcgagacc agcctggaca acatggcaaa accccatctc tactgaaaat ataaaatagc |
| 15601 | tgggcgtggt ggtaagcacc tgtagtcctg gctacttgRg aggctgaggc gtgagaatcg |
| 15661 | cttgaaccca ggaggcaagg gttgcagtga accaaaattg caccactgca ctgcagtcta |
| 15721 | cacgacagag caagactctg tctcaaaaaa aaaaaaaaaa Watggaaaag tgcttgtaca |
| 15781 | caaatgatgt cctggttctg gtttcaggta ttaattgaag aaacagatca attatggaaa |
| 15841 | gttcattgtc accgagactt taaggaagaa agacccgaag agtatgagtc gtggcgagag |
| 15901 | atgtacctgc ggcttcagga cgcccgagag cagcggctac gagtactaac aaagaatatc |
| 15961 | cagttcgcac atgccaataa gcccaaaggt aacagagacg ggagagctgg gggagaactg |
| 16021 | gcaggatgag tgttctggtc aatagcaggg tgttggtggg cttggctggt gtaggactgc |
| 16081 | ttgcccctac atcaggtagc agggtgggta tttcttctgc caccgcccat tctaccctac |
| 16141 | aagtggggtt tcagggcact caaaaagccc gtggattatt taaataacag tagcatctct |
| 16201 | gctcaagaag gcagatgttc atctccataa ttccacaggc cgacaagcaa agatggcctt |
| 16261 | tgtcaactct gtggccaagc cacctcgtga cgtccggagg aggcaggaaa agtttggaac |
| 16321 | gggaggagca gctgtccctg agaaaatcaa gtaagatctg tgttcttacc tggcttttgt |
| 16381 | gtgctatcaa cccttagagg tagccatagc ggcaggaagc aaattgcttt tggtgaaagt |
| 16441 | gaggcagtgc agactgtggg ctctggaggc agacctcatc agctcagcac ttccctcacc |
| 16501 | tccctcagcc tccgcttctt ggactgcagt gcaaggtgat aattacccct gccctagggt |
| 16561 | agtcaagatt aaatggagag caaagcctgg cttatgggaa acattccctt atgctttccc |
| 16621 | ttttcccatc taaacaaaaa acacatttct gcaatgattg ttattttgtc taagccacat |
| 16681 | tagccttttc aaaatacaga tttataccaa aacagtgaaa agttagttga gattatcatt |
| 16741 | ttttctgaaa ggataatgga gacatctctg agccaccacc agaatccagg gtgatagggt |
| 16801 | ccattggtca aagccacaaa cagggtgagt catcctcaca caggccagct tcaggctgtt |
| 16861 | catgctcagg gcagatttct tgtgctcatg cctaaccagt gttgtcctgt gttgcaggat |
| 16921 | caagccagcc ccgtacccca tgggaagcag ccatgcttcc gccagtagYa tcagctttaa |
| 16981 | ccccagccct gaggagccgg cctatgatgg cccaagcacc agcagtgccc acttggcacc |
| 17041 | agtggtcagc agcactgttt cctatgatcc taggaaaccc actgtgaaga gtaagtaact |
| 17101 | tggagttcct gctcaaatat tatYtcagca ctagttttta actctcaatg tcattattca |
| 17161 | ctgtattaca ttttgtggac tgcatgttgc atgtacatga tgccttgcac atagctggtc |
| 17221 | tgaatgtcag tccctccttc cccctgcttt gtccatagtg cattatatct cttaaatagt |
| 17281 | tatagtgagt ctgggtgcag tggctcatgc ctgtaatccc agcactttgg caggctgagg |
| 17341 | caggaggatc ccttgaatcc aggagttcRa gaccagcctg gacaacataa tgagaccctc |
| 17401 | tacaaaaaat taaaaaatag ggcatagtgg tacacacctg tagtcccacc tactcaggag |
| 17461 | actgaggtgg gaggattttt tgagatggag tctcgctctg tcacccaggc tggagtgcag |
| 17521 | tgScacaatc tcagctcact gcaagctctg ccttccaggt tcatgccatt ctcctgcctc |
| 17581 | agcctcccaa gtagctagaa ctacaggtgc ccaccaccat gcccacctaa ttttttctat |
| 17641 | tttttagtag agacggggtt tcaccatgtt agccaggatg gtctcgatct cctgacctcg |
| 17701 | cgatctgccc gccttggcct cccaagtgct gggattacag gcgtgagcca ctgtgcccgg |
| 17761 | ccgaccctgt ctctggaaaa aaaaaaaaaa aaaaagtgaa agtagaaaat agtatctggc |
| 17821 | caggcatggt ccctgtctct attaaaaata gaaaaattag cccaccatgg tggcatgtgc |
| 17881 | ctataatctc aggtactcgg gtggctgaga caggagaatt gcttgaacct ggcaggcaga |
| 17941 | ggttgcagtg agctgggatt gtgccactgc cctccagcct ggatgacaga gtgagacact |
| 18001 | gtctctaaat aaataaataa atatcattgt gaatgaccat tttggcactc ttcttttttc |
| 18061 | acctaaatat aaactgactt ctctaggcaa taccacagtg ttcataatta ctaatattaa |
| 18121 | cagctaaatg acaggccctc ttactgtttt tttatagWct tataactcta gcttccaaat |
| 18181 | atttattctt tatatctaat tgggtcttcc aatttatttW tttWtttttt tttttttttt |
| 18241 | tttttttttt tttttggaga cagagtctca ctctgtcgcc tgggctggag tacagtggcg |
| 18301 | tgatctcggc tcactgcaac ctccacctcc caggttcaag cgattctcct gcctcagcct |
| 18361 | cccaagtagc tgggattaca ggtgcccgcc actaagccca gctaattttt tgtattttta |
| 18421 | gtagacacgg ggtttcacca tgttggccag gctggtctcg aacccctgac cttgtgattc |
| 18481 | gcccgcctca gcctcccaaa gtcctgggat taccagcgtg aaccaccatg cctggccRgg |
| 18541 | tcttccaaat atttattctt tatatctagt tgcgtctatt ttgtattttg ttaactttat |
| 18601 | taccatagac tatccccagt atcctaaggg gatgtcctaa cttaagtctt attctgtatt |
| 18661 | aattgcaggg cccatgggag ggtgggagtc ctggcttgag ctacagctat gctcagatga |
| 18721 | accattcagg aggcaggctg gccttcctta ggcagtactt gcatagccac cttagacctg |
| 18781 | tttctacact ggacagattg ggggtagacS tgctgtttac tggtttggct atttgtgagg |
| 18841 | caaagttaag ttatgactga gtatggcctt tagagtctgg caagggccgg ggcacagtgg |
| 18901 | ttcacacaca cctgtaatcc cagcactttg ggaggctggg gtaggagggt gcttgagccc |
| 18961 | aggagatcga gaccagcctg agcaacatag tgagaccctg actctacaga aaataaaaaa |
| 19021 | aaattagcca ggcatggtgg cacactcctg tagtctcagc tactctggag tctaggaggt |
| 19081 | agaggctaca gtgtgctgtg attgtgccaa tgcaccctag ccagggtgac agagtgagac |
| 19141 | tctttcctta caaaaataaa aacaagaaag tctgacagag attgaattgt aattctgtac |
| 19201 | cttactgtac aaccatgggc aagttggcca ggctctctga gccttggtgc aggtggttag |
| 19261 | agagagtggt ggtcatgccc ctggcaggtc ctccgaaagt gtctgctctg tgctgttgct |
| 19321 | tttattgcat tgtgaacaga ggagatagtt actacttgta tctctgcctg gtggtgcaca |
| 19381 | gccaagaata gcagagctgg ctttgactgt aaactactgg gggtgtgtct aagccgccac |
| 19441 | agatctgaga cagctgcctt gtttctcttt tcacagaaat tgccccaatg atggccaaga |
| 19501 | caattaaagc tttcaagaac agattctccc gacgataaac tgaggacttg ccttggaaat |
| 19561 | ggaatctggg gaggcaggaa tacaaggaca gtgggggttg gggaatggaa ttctacagga |
| 19621 | gactggagtc ttgctttgtg gatccttttg gtctccgagt cctgcagtct gcaggtgctg |
| 19681 | cccctgggaa cctgcgtgcc acagccccgc ctccctgcct ggagcacact ttagaattct |
| 19741 | gaagatgtga agcctctgtc tcactgagga ttttaaaggt caattatact tttgttgttc |
| 19801 | attagcatct ttgtaaacta taagacgtag ttttaattaa taaatattgc ccccagattg |
| 19861 | tatttatatt atcccccagg tttgtttttg ttttttgtcc tctaccacac atttagcctt |
| 19921 | ttatcttcca ggtccttatt aaaatcagat gaaagcctag tgaaagccag tctcctgccc |
| 19981 | cagctcagct ctgtgtggac tctggtccag acagaggact gggcatctcc agagcctgca |
| 20041 | cagtacctgc tgcacgtagg gcaaggaatg agcactagac cgcctgtccc caagggagcc |
| 20101 | tcagtggggc gacagggtgc tcggcggact ccacctcagg ccctccccac tgttgctgtg |
| 20161 | cattcctgtg caggtgcatc tctttcttac taactggtat ttattaaggg aggtgctctg |
| 20221 | taggtctgga gcctttccct catccttttt gcgagtcccc acctttttgt tttttttttt |
| 20281 | tNctttgagg ctcactagag gacgcagaac cttgggagat tgatttgcac agaactcccc |
| 20341 | acctcccact tttacaattt ccagtttctg attgaaaatt ttagggtttc tccccactgc |
| 20401 | ccttccctat ctttccttcc cctcaacacc atgaaggaaa aacacacacg gcagggcttt |
| 20461 | ttgtagccct gaaggcaact ttagacattt aaaaKccagc actttaatct cttgttctct |
| 20521 | gtgaatcact atgagaagtg aatggtttta aaggctgtaa tgctatgttg gaaattggtt |
| 20581 | tgttttgcct tttattgaaa aggtaagatc atgtgattgg aagaacacaa ctRttggctt |
| 20641 | gggaagagga ctttgctgYt gaagtgtttt ctaccttctg agtgtgttta aggcaggatt |
| 20701 | tggagggaag gaccagctta gggagagtgt ctgagccaca gcgtcaggat gggggaaacc |
| 20761 | acatgggatc catcaagttc cagttgaaca ggagcaagat cagaacttag gagggcagtg |
| 20821 | tcagctccct tgttggctgt caaggaacac cgatctagta gaaacccact tggttgtgac |
| 20881 | ccaggtagag gtagatgcca tacatttgag atatgcgtcc ttaaggaacc tgacaagcag |
| 20941 | actgaaggga tggtaagtgt gacagcctga taagttttct caaagcccag gatacagagc |
| 21001 | cagtgttttc tgtaactgga gacctcagtt aggccaactt cgaattccag agcaacgtag |
| 21061 | gaagtctatt cagcagaaac tcgacattgt tcagtgtgta ttgctgtgca gggtgcctat |
| 21121 | tgtgacagga cacaaatgtt actatgtttt aatttgctat atttttgaat gggtaaagca |
| 21181 | ttactttact tctcttggtt acttgtacca ccattccacc cctatcccta gcctgcccca |
| 21241 | caaatctaat attaggaagc ctcttaactg aaaccaaatg aacatttggg tcaggtgcca |
| 21301 | gatgtctgct gcctagaata gctttttcta ggtgtctacc accttgaatt tatctcttaa |
| 21361 | ctgtgtgttc aagtctttgt cattgaaact agtttttcat atcttagatt cagttgtgta |
| 21421 | tgatttaatg tcccttatta ggagtcttta ggcagggagg gaagaaaaaa cagatttgtt |
| 21481 | catagcaatg tcagtatcca ttttggcaca taaagatttt tgatgagccc tgtttgcata |
| 21541 | gagccagatg ttttcccctc ccccaagagt atctacatca gggatgtgac ttggtgcgaa |
| 21601 | gaRtcagggg aaagaggaaa aacccaattt ctaaatgacc tccttgccca gcttactaaa |
| 21661 | atggctgcag agcagacaca ggatgaattt gaacctgaca caggatgaat ttgaaccttt |
| 21721 | ggtctcattt atggaaaaac ttgtgcaatt ttttttctgt gctacactac atacaaatca |
| 21781 | ccaaattaca aattaccctt ttgtgatcct tggtgtactg agcagtttct ttggggcttt |
| 21841 | ttctttctgg gaagcgggag ggaaaggagc aaggtgtcat cctgctcttc atttgtattt |
| 21901 | tggtcccaaa atgtaaatac aattttctat gttacttttt tgtggtaact accgagatga |
| 21961 | atattttaat tagataagtt atatgaaaag gaaaattcca tgtctaaata aaaaacaaac |
| 22021 | tccatatact gtgtcttcct tacttatagc atcagtggaa agaagttaga aaatgcctga |
| 22081 | ttcttggact gtttaaggca gagttttttt ttttgttttt ttttttgaga tggaggaggc |
| 22141 | aatctcagct caccgcaccc tcctaaggca gagatcttaa tcagcatcct tgggctgggc |
| 22201 | cttaaacgat gccgaattta gtagtaagag gaatttcaga cggaaagaat tctgtgagca |
| 22261 | aagggcggga actcagcatt tgtggggaac aggcactgtc ccttattata cttggaaccc |
| 22321 | tggtaacccc atcttaaaat tagaaactgg ttcaaagggg tgaaatgggg ccgggcatgg |
| 22381 | tggttcatat ctgtaatccc aacactgaag ggcggaggca ggattgcttg agcccaagag |
| 22441 | ttcaagacca gcctgagcaa catattgaga ccctgtctct agaaaaagat taaaaattag |
| 22501 | ccaggtgtga tggtgcacct atagtcccag ctatgtgaaa ggctgaggtg ggaggatcac |
| 22561 | ttgagcttgg gaggtcgagg ctacagtgag ctgtgattat gctactgcac tccagtctag |
| 22621 | gtgacagagc aagaccagcc tgggcaacat ggcaaaaccc catctctttt tttgtctcaa |
| 22681 | aaaaaaaaca ggggtagggg gttaggaggg actgtggctc atgcctgtaa tcccaacact |
| 22741 | ttgggaggcc aaggagggtg gattgctgga gttcaagagt ttgagaccag ccttggcaac |
| 22801 | atggcaaaac tctatctcta caaaagatac aaaactagct gggtgtggtg gcacacacct |
| 22861 | ttggtcccag ccactcggga ggtagaggtg ggaggatggc ttgagcccag gaggttgagg |
| 22921 | ctgcaataag ccatgattgc cactgcagtc cagcatgggc gacagagcaa gaccctgtct |
| 22981 | ccaaaaaaat aaaaaacaaa cggggagggg ggggaaagga gtgaaattaa catggccaag |
| 23041 | ttgcgtagct aataaagtgg caaaaggaag gatttgacac cgtgattctc taactgcaag |
| 23101 | gtttgtgcca ctctgccttt cagtttccac gggctgtgag ggaccagtag gtctcagctt |
| 23161 | acacgggcgt tctaatgagt acagaaggaa catgaatgca ggaaagtcgt gccagcaaag |
| 23221 | acttaatgag aacctgctgt ggtggctctg gctgacctgc aggagcttta gcacttgcta |
| 23281 | atggagccat ggatttggct tcaaccctgc atacagcaag tggattggac cttccatcaa |
| 23341 | acatacaaat ctgtccttta tcgattaatt aaacatttgc tgagccagga cctgtattag |
| 23401 | accttaggat atggagtaat agactctgac acttaacagt tagggaggag taactgactt |
| 23461 | tattgattat gcacctacta ggtgccacca ttccttcttc tgtacactgc ctctgttatc |
| 23521 | acaatgatcc tgtgaggtaa acagtcatgg ccccatttta aaaatgacaa cttaatcctg |
| 23581 | gtaaatccag gggattaaaa aataaaaatg agaacttaag tcaccatagt gtctgtagaa |
| 23641 | ataaatttta aaaaattgtt ttaatgaaaa tgagaaaaac cgaagcacag agagatgaag |
| 23701 | aacttgccta agttcacaga atcagtgagc agtggtgcta tgtattggcc tgactccaaa |
| 23761 | acacatgctc tcagtcacat ttaccatctc atacactgac atgcataccc aatttggcct |
| 23821 | ctgaagcaaa gctgacctcc aaaacaaaag atgcctgagc caaggccaga gtcgtttctg |
| 23881 | tttttttttt ttaaattctt tttttgtgat ggagtctcgc tctgttgccc aggctggtgt |
| 23941 | gcagttgcac gatctcggct cactgcagcc tcagcctcgc gagtagctgg gattacagac |
| 24001 | atgtgccacc acgcccagct aatttttata tttttagtag agacagggtt tcaccacgtt |
| 24061 | ggccaggctg gtctScaact cccaacctca agccatctgc ccgctttggc ctcccaaagt |
| 24121 | gctgggatta caggtgtgag ccaccgtgcc cagcctaaaa attctttatc ctcaagttca |
| 24181 | acagtcattt Ytaagaatga tatcttttgt ctgcttcatg taactgatgc ccagcagaat |
| 24241 | cctacctggc ttatcaactt gacagatgcc cccatcacag ggtttctcat ccactttcat |
| 24301 | ctcttcctgg ttatttgcta aatggtgaaa cagtttgtct tcagaggccc ctgaacttct |
| 24361 | ttctctcact ctttttgaga cagtctcact ctgtcaccca ggctggagtg cagcggtggg |
| 24421 | atcttggctc actgcagcgt caacctccca ggctcaagta ttcctcccac ctcagcctcc |
| 24481 | caagtagctg tgactacaga tgtgcaccac cactcccaga taattttttg gtattttttt |
| 24541 | gggagatgga gttttgccgt gttgcccagg ctggtcttga actcttgagc ttaatcactc |
| 24601 | taccttggcc tccaaagtgc agggattaca ggcatgaggc acctcgcccg gccccctgaa |
| 24661 | cttctctatg atgtctattg tctcaactct tttcaaagac taaggaaagt tccagccatc |
| 24721 | aaaattttat atcactgtat ctgtttctca agcacagagt cttgggttgg ggcctcagaa |
| 24781 | tatgcattta aaaagatgca actcaagtgt tatggactaa atgtttgtgt ccccccaaaa |
| 24841 | tgcctatgct gaaatcctaa gacccagtgt gacagtatta gataggcctt tggaggtaat |
| 24901 | taggtcacgt gggtagaggc ttcatgaata ggattctccc tcataagaga aatgagagtt |
| 24961 | gctcttgctc cttgttctcc accatgtgag gaaatgagag gacggcagtY tgcaaaccaa |
| 25021 | gaagagggcc ctcaccagac acagaattgg ccagcgcctt gatcttggac ttccagcctc |
| 25081 | tggaactgtg agaagtaaat gtttgttgcc taagccagtc agtYtatggt aagttgttac |
| 25141 | agtaScctga aatgactgag acactaagcg attttttttt tttttttttt tttttttttt |
| 25201 | tttttttttt tttttttgag gKgaagtctc actctgtcac ccaggctgga atgcagtagc |
| 25261 | ttgatcactc tcggctcact gcaacctYtg cctcccgggt tcatgtgatt ctcctgcttc |
| 25321 | agcctcccta gtagctggga ctacaggcgt gcaccaccaa cttttgtatt tttagtagag |
| 25381 | atgggtttca ccatgttggc taggctggtc ttgaactcct gacctcaggt gagatcaggt |
| 25441 | gagatccacc tgcctcgacc tcccaaagtg ctgggattac aggcatgagc cattgtgcct |
| 25501 | ggcctgacac taagcatttt ttttttgggg ggatggagtc ttgctctgtt ccccagactg |
| 25561 | gagtgcagtg gcgtgatctc agctcactgc aacctccgcc tcccaggctc aagcgattct |
| 25621 | cctgcctcag tctcccaagt agctgggatt ataggcatgc atcactacaa cccggctaat |
| 25681 | ttttgtattt ttagtagaga cggggtttca ccatgttggc caggctagtc ttgaactcct |
| 25741 | gacctcaaat gatccacccg cctctgcctc ccaaagtgct gggattacag tcatgagcca |
| 25801 | ccacgcccgg ccgacactaa gcaattctta aaatgtacac tgaagtttga gaactgctcc |
| 25861 | agagaatttg ccacagtgga atagctcttc acagcaggtc cctaggtcag gactaggaag |
| 25921 | cttgttgatt tagtagtaag caaatacttg cattgcttcc cactctgtgt cactgactaa |
| 25981 | gtcagacatg gcaggctggg gacagtggct cacagctgta atcccagcac tttggggggc |
| 26041 | caaggtgaga gattacttga gtccaggagg ttgaaactaR cctgggcaac atagtgagat |
| 26101 | cttgtctcta aaaaaataaa atattaggcc aggcgcggtg gctcatgcct gtaatcccag |
| 26161 | cactttggga ggctgaggcg ggcagatcac ctgaggtcag gagatcaaga ccagcctgac |
| 26221 | caacatggag aaaccccatc tctactaaaa atacaaaatt agccgggtgt ggtggcacat |
| 26281 | gcctataatc ccagctactc gggaggctga agcaggagaa taccttgaac ctgggaggtg |
| 26341 | gaggttgaag tgagccaaga tcatgccatt caaaataaaa taaaaataaa acaaaataaa |
| 26401 | ataaaatatt agttgggcat ggtggcatga acctgtagtt ctagctactc aggaggctga |
| 26461 | ggtgggagga ttgcttgaac tcggggaggt tgaggctgca gtgagccaag atcatcccac |
| 26521 | tgcgcaccag cccgggtgaa agagcaagac cctgtYtcaa aaaagaaaaa agaaaaaaaa |
| 26581 | aaaaaaggcc aggcatggtg gctcatgcct gtaatcccag cactttggga ggtcaaggca |
| 26641 | gatgaatcac ctgaggccag gagttcgaga ccagcctgac ccacatggag aaactccgtc |
| 26701 | tttactaaaa atacaaaatt agctgggcgt ggtggtgcat gcctgtaatc ccagctactt |
| 26761 | gggaggctga ggcaggagaa tcgcttgaac ccaggaggtg gaggttgcgg tgagccgaaa |
| 26821 | ttgtgccact gcactccagt ctggacaaga gcaaaactcc gtctcaaaaa aaaaaaaaaa |
| 26881 | aaaaaaagag acatggccct tgccctcatg aagctttcat ctagtaggga gacagttatc |
| 26941 | aaataactgc acaaataatt cttttttttt tttttttttt tgagacaaag tcttgctcta |
| 27001 | ttggcaggct ggagtgcagt ggcgcgatct tggctcactg caacctccgc ttcccggatt |
| 27061 | caagcaattc tcctgcctca ggctcccgag aagctgggac tacgagtgtg cgccaccatg |
| 27121 | cccagctaat ttctgtattt ttggtagaca cggggtttta ccatgttggc caggatggtc |
| 27181 | tcgatctctt gacctcgtga tctgcccgcc tcggcctccc aaagtgctgg gattacaggc |
| 27241 | gtgagccacc gcgcctggct tttttttttt tttttttttt tttttttgag acggagtctc |
| 27301 | actctgtcac cagggctgga gtgcagtggc gcgatctccg ctcactgcaa gctccacctc |
| 27361 | ccggttcacg ccattctcct gcctcagcct cccgagtagt tgggactaca ggctcYcgcc |
| 27421 | accacgctcg gStaattttt tgtattttta gtagagacgg agtttcaccg tgttagccag |
| 27481 | gatggtcttg atctcctgac ctcgtgatcc acccacctcg gcctccaaga gtgctgggat |
| 27541 | tacaggcgtg agccacggcg cccggccaca aataattctt taaagtacag gtttctgggc |
| 27601 | cctactgaag aaatatggtt gaataggtca aaggtaggat ctggaaacag gtacgctttt |
| 27661 | gtaggcttcc cccaatgaac tcgatgagcg gcctggtttg ggaatgtcta gtgtagtgga |
| 27721 | aaaagcatgc atgacctttg ggatgagacc caggttcaaa tcctgcctcc accactccca |
| 27781 | cacctctctg agcctcagtt tcctcattgg taaaatggga attatgacac tctgcaggaa |
| 27841 | ttcttataag gattcaatga cagaagggga gccacatttt agtgtccctc cttctttccc |
| 27901 | tctaaatgtg gaatgaaaga aaaaggtttt gttttttact gatgggaatt caaacttcag |
| 27961 | ggtcacaagc taagaactat tttgaggtca acaagtcctt cctgataaat gaaatcttag |
| 28021 | tgcctgcttc ctcagagtta gtttgacttt tctgttttgg ggccccattc taattttttc |
| 28081 | gggtccctta aactgcagct tctaacaacc actaaaaagg aagaccacct aatttcttac |
| 28141 | ggccacaaac tcaaatacca ggggtttggt atgtgacagc aggctgtgat gggaacagaa |
| 28201 | agaaaatgga gctattcaca cagctccact ttattgcttc cgggttgaag acagggccca |
| 28261 | gtgttgccac ctcttctgga tttaaagaaa atggccggcc aggtgcggtg gctcatgcct |
| 28321 | gtaatcccag aactttggag gtgaggcagg tggatcactt gaggtcagga gtttgtgacc |
| 28381 | agcctgccaa catggtgaaa ctccgtctct accaaaatat acaaaaatta gccaggtgtg |
| 28441 | gtggcgtatg cctgcagtcc cagctacttg ggaggctgag gtgggaaaat ctcttgaacc |
| 28501 | tgggaagcaa agtttgcact gagtgacaaa gctccagcct gggtgacaaa gtgagaccct |
| 28561 | gtctcaaaaa aaaaaaaaaa aaaaaaaagg aaaaaggaat aggctcagca gtagttctca |
| 28621 | actagaggtg actttgttcc cagtatgaca cttggcaaca tctggagaca ttttggttgt |
| 28681 | cgctgttgtt ggggagctat gggcatctag tgggcagagg ccacggatgc tgctatccat |
| 28741 | gctacaatgc acaagacagc gcccctcccc caacaaagaa ttatctggcc gaaaatgtaa |
| 28801 | atagagccaa ggttgagaag ccttggacat ggttctgttg cagcaacaac cctcaaatct |
| 28861 | cagtggcttg caacaacaga ggtctcaccg cctgtccatt gtgagtctga cagggatctg |
| 28921 | ctttatgtca tcctcactca ggggcccagg ctaagggagg tgccattccc atgcttgtaa |
| 28981 | gatcttcgag gcaagtagag aatattaata tattattaac attaattata ataataaaaa |
| 29041 | ataatattaa ttacaatatt gatataactg ttaagttttg tctagaagtg acatctatca |
| 29101 | ttgccatcat attgcattgg ctgcacctta attcatgggg tcaaggaagc acaatccatc |
| 29161 | catgttcctg gatgcagaga attgggaata tttgctaaca gcattaaaga ttccaaaggt |
| 29221 | tgggtgcacc aaaacctcag agatcaccac taaagaactt attcatgtaa ccaaacacca |
| 29281 | cctgttcccc caaaacttat tggaaaaaac taaaaatcca gagaaaaatg tatgctgacc |
| 29341 | aaaaaaaaca aaaaaacctc caaaggtaat catgacagcc tgggctctag gtaatccaca |
| 29401 | aggctcagag aagggcagct aagggcacag gctgtggagt cagacgtcag acttggcttc |
| 29461 | aaaccccaca ctttctagcc atgtggactt gagcatgtca cttaatctct cagtttcttt |
| 29521 | ctctaaaaat gaggaaaata ggctgggcgt ggtggctcac acctgtaatc ccagcacttt |
| 29581 | gggaggccca ggcaggcgga tcacgaggtc aggagctcga gaccagcctg gccaatatgg |
| 29641 | tgaaacccgg tatctactaa aaatacaaaa attaggccag acgtggtggc ccacgcttgt |
| 29701 | aatcccagca ctttgggagg tcaaggaagg cgggtcacga ggtcaggagt tcgagaccag |
| 29761 | cctgaccatc atggtgaaac cctgtcttta ttaaaaatac aaaaattagc caggtgtggt |
| 29821 | ggtgtgagcc tgtaattcca Rttactcagg aggaagaggc aggagaattg cttgaacctg |
| 29881 | gaaggtggaa gtcgcagtga gccgagatta cgccactgca ctccagccta ggtgacagag |
| 29941 | tgagactcca tctcMcaaaa aaaaaaaaaa aaaaaaaagt ctgggtgctg tggctcacgc |
| 30001 | ctgtaatccc agcactttgg gaggccaagg tgggcggatc acgaggtcag gagatcaaga |
| 30061 | ccatctctac taaccctgtc tctaataaaa atacaaaaaa ttagctgggc gtggtggtgg |
| 30121 | gcacctgtgg tcccagctac tcgggaggct gaggcaggaR aatggcgtga acccgggaga |
| 30181 | tggagcttgc agtgagccga gatcatgcca ctgcactcta gcctgggcga cagagtgaga |
| 30241 | ctctgtctca aaaaaaaaaa attaattaat taaaataata ataataataa ttaSctgggt |
| 30301 | atggtggtgt gcgcctgtag tcccaggtac tcaggaggct gaagcagaag aatcacttga |
| 30361 | acccaggagg tggaggttgc agtgagctga gattgtgcca ctgcactcag cctgggtgac |
| 30421 | agagcgaggc tccatctcaa aaaaaaaaaa aaaaaaaaaa acaagggaaa ataaatcaac |
| 30481 | cttgcagtgt taaaatgctt agaacagtgc ctggcacaca gaaacagctc aggacatgtt |
| 30541 | agccaggatg atgatgatga tggattaggc caggcgccaa tcacggagtg ggcatttagt |
| 30601 | aaacagttgt tgttataacc taagctctga ggaaaatgac ttgtccaagg taacacagct |
| 30661 | ggtaagtgca gcatggtgga gaagagccta agctctgggg tctcagcctg gcacatcgta |
| 30721 | aatcataaat attaaataat atttggagga ggttcagtgt ggttaagaac atggactcag |
| 30781 | gccaggcacg gtggctcaca cctgtaatcc tagcactttg ggagcctgag gcgggcagat |
| 30841 | cacgaggtca ggagatcgag accatcctgg ctgacacggc gaaaccccgt ctctactaaa |
| 30901 | aatagaaaaa attagcccgg cgtggtggcg ggtgcctgta gtcccagcta ctctggaggc |
| 30961 | tgacgcagga gaatggcgtg aacccgggag gcggagcttg cagtgagctg agatcgcgcc |
| 31021 | actgcactcc agcctgggSg acagagcgag actctgcctc aaaaaaaaaa aaaaaagaac |
| 31081 | atagactcgg tagctcacac ctataatccc agcattttgg gaggctaaca ggggcgcatc |
| 31141 | ctttgatccc aggagtttga gaccagcctc ggcaacatgg caaaactccg tctctacaaa |
| 31201 | atatacaaaa attagccagg catggtgatg tgtgcctgta gtcccagcta ctcaagaggc |
| 31261 | tgaggcagga ggatcactta agcctggggg tgtcaaggct gcagtgagct gagatcacga |
| 31321 | cactgcactc cagtctgcgt ggcagagcaa gaccttgtct caaaaaataa ataaataaaa |
| 31381 | ataacatgaa ctcccgaggc tgactgccgg gttcaaatcc tagctctact actgaccacc |
| 31441 | tgtatgaact tgagcaagta actcagcctc tctgtgccca gttttctcat ctataaaatg |
| 31501 | aagctaataa taaaacacct gcaattagga aaattaaaaa ataacattag taaagccctc |
| 31561 | gggacccata tggttgttag ctgctacagt attactatgc cctcagttcg ccacaaagaa |
| 31621 | gcattcaaaa gtaacagaaa ataattaaca gtatcatttc acccacccaa ttgagatagg |
| 31681 | agagaagaaa atggatacaa attcagcaat ccttgagcta agaaaatgtt tacagccagg |
| 31741 | ggtggtcgct caggcctcta gtcccagcag ttagggaggc tgaggccagc agatcacttg |
| 31801 | agtccaggat ttcgagacca gcctaggcaa tgtgatgaaa ctccatctct acaaaaaata |
| 31861 | caaaaattac ctgggcatgg tggcatgcac ctgtagtccc agctactcag gaggctgaag |
| 31921 | tgggaggact ggttgagccc aggaggcaga agttgcagtg agccaagact gtgccactgc |
| 31981 | actccagcct gggtggcaga gtgagaccct atctccaaaa aaaaaaaaaa gtttataaat |
| 32041 | ggagcaatgg gaaaaaactt ggatgctttt Yttacctgcc ccagggtagt cctagaaatc |
| 32101 | agtgagaatt cccatttgtc ctcagagatt cctgcaacat aggagtgcaa gagaggagac |
| 32161 | agtaagcaga ggcacaaggc attactccca aatcacagct aagccaccct gcaaccctgg |
| 32221 | ccggctcctt acaaacccag ctcccttagc aactgcagtt tcccatccaa ccagacgagg |
| 32281 | gcgccgagct gacagtcttg ctgctggcaa tagtgatagc cgctgtgaaa gcacagtaga |
| 32341 | tgctcgtcca cttgtcgagg agaatgctga attcttacca cttttgcacc actgtaaagt |
| 32401 | cacaaagtca taagtcgaac caccataaac tggggattat ccgcagtccc cccttatctg |
| 32461 | tgggcgatat gttccaagac cctcagggga tgactgaaat agtactgaac gctatatata |
| 32521 | cagtactatg cttttttcta tacatacata cctatgatta catttaattt ataaattagg |
| 32581 | cacgatgaga ttaaacaact aataataaaa tggaacaatt atagcaatat gatgtaatac |
| 32641 | agttatcagg cactgtgctc ataacttttg cagtttcatg tgtggcagca aaactagcag |
| 32701 | gtatttcttt ttccttcttc ataatttcac agatagaaga ttcgttctta ctacagatct |
| 32761 | tagcgacctc agcacacttt attttttctt tccttattat aacttttacc ttttcattta |
| 32821 | aagaaagaac tttatggctt ctctttgcca tatccaaatt gctagcatga ctactcttgc |
| 32881 | actttggggc cattattaag tcaaataagg gtgacttgaa cacaagcatt gcaatatcac |
| 32941 | ggcagtcgat ccgataacga acagggcact tacttagtaa gtgactaacg aacagggagt |
| 33001 | gtatacagcg tggaggcact agacaaaggg acgattcagg ttccccaggt ggacagagcg |
| 33061 | ggacagcaca agacttcatc acgctactca ggacagcatg caatttaaaa tttacagatg |
| 33121 | gtttatttct gaaaatttcc atttaatatt tttggacagt ggttgactgc atgtaactga |
| 33181 | aaccacagaa agcaaaaccg agtcaggggc aggggagggg ggcggctaca gtaatagcct |
| 33241 | ttccctgtgg accagatgtt ataccaaaca ctttcatgtg ttatttcaca ttactgtcat |
| 33301 | agcactccac aacataggta ctgttatgat tcccatttta tgtaagacaa atctgaggcc |
| 33361 | tagaaaaata aataaataaa aagaaaaaag aaaaaaatct gagacctaga gaggttaagc |
| 33421 | aatttgccta gcaggccaaa cctaatattt gcaggacagg agcaagaata cagctgaatg |
| 33481 | catacttaaa aagttataaa tgaccctaat gaactattaa ataaaagaca atctattctg |
| 33541 | ctatgtcaat aaataaacct tcacaatgac ctggaagacc acattcacat ttttgggcct |
| 33601 | ctcagagttc tgtactagaa ggtggtgaga ggcagagaac aggaccctag ccctgcccca |
| 33661 | ttctctccat atctctaaat ctgtgtgcct ggaagggYct tgtgtccacg catgcagagg |
| 33721 | tcctaacctg tgtgctcaca ctttgttcta ggggttctaa attgaaaagg tccctcaggt |
| 33781 | atccagcaca atgaatgaca aaagagacca aagcacaaca aaaatcatca cattctagaa |
| 33841 | taccaagaga agatcttaaa agcttccaga aaacggggtg gggtgggggg aaaacccata |
| 33901 | aaaagaatca ggattcagaa cagcattgta tttatcaaag ccagaagaaa accagagcaa |
| 33961 | tttcttcaca attatgaaag aattctatcc acagccaaac tactaattaa gtacagtaga |
| 34021 | ataaagccat tttcagtgcc acttaacatg tatggcagca tggtagccag ccactaaggt |
| 34081 | ggcctctaat gatttctacc tcctggtatc tgcactctta tgaagttccc gcacacactg |
| 34141 | aatatggctg acctgtgaaa ccatgggatg ttgcaaaatt gacaatatgg gacttctgtg |
| 34201 | gctaggtcat aaaagacatt ggtggtttct accttgccct tagatcactt gttctgctga |
| 34261 | aaaccagctg ctgtatcatg aggacactca aacagtacta ttgagaggtc catgtgttaa |
| 34321 | ggaactgggg cctcctgcca agagcctgca caaacttgcc agccggggct tctccagccc |
| 34381 | tggccaagcc ttcatatgat ttcaggctca tgagagaccc caagccagaa ccacccagct |
| 34441 | aggacgctct caaattccta actcacagaa actgtgagat gataaatgtt aattgttgtt |
| 34501 | ttgaggagag aggccatttc tcttactgtc tctgaagaga aggaagaagt aaaagctgaa |
| 34561 | aaacaacagg aatgaagtca gtggcaagac caactgatgc cacttaccag gcctgaggtt |
| 34621 | aaagattaac gcccccaccc taaccacatg tgttacctat agatcacttg ctcaatctat |
| 34681 | cacaaccctt tcacgtggac ccccttagag ttataagccc ttaaaagggc caggaacttt |
| 34741 | ttctttggag agcttggttc ttgagacaca actctgccaa cgctcctggc cgaataaagt |
| 34801 | ctcttccttc tttaacctgg tgtctgacag gttttatctg cagcttgtcc tgctacagtt |
| 34861 | ttaagccatt aagtttggga ataatttgtt acacaacaaa agacaactaa tacagaccta |
| 34921 | ctgggagggg aggagccaca gtattttgca gttcttccaa tcaggaggtg aagtctacag |
| 34981 | tctagctctc cactctagaa tttggatttg gctgatgggt cattatagaa agattataga |
| 35041 | tgtaaggctt aaaaagtgct tgtgctggca gggcgcagtg gctcatgcct gtaatcctag |
| 35101 | cactttggga ggccgaggtg ggcagatcac ctgaggtcag aagtttgaga ccagcctgcc |
| 35161 | caacatggtg aaaccccatc tctactaaaa atacaaaaat tagccaggcg tggtggcagg |
| 35221 | cgcctgtaat cccagctact cggaaggctg aggcaggaga atcgcttgaa cccgggaggg |
| 35281 | ggaggttgca gtgagcggag attgtgccac tgcactccag cttaggtgac agagcaagac |
| 35341 | tccatctcaa aaaaaaaaaa aaaaaaaaaa gtgcttgtgc aattgagtct tgccctctct |
| 35401 | tgctcctggg aactctgcaa ttatcaccat gtaatttagc taggcttgtc tgctggagac |
| 35461 | acgtggtccc atcatcccag ctaacagcca gcatcaagca gacgagagtg agaccatccc |
| 35521 | aaactttcca gcccaagctg aactggccca gaaaMgctaa tccacaggat catgcgaatt |
| 35581 | aatcaatgtt tgttgtttaa agctatcaaa gttttagggt aatctgttac acagcataag |
| 35641 | ctaattgata cagcatattt actttgtgtg cacctcccgc ctcaacctcc ccaccctaac |
| 35701 | caggaaacta ctgatgtgtt acaagaacac tacaggatgt gttcaaagcc tggttacctc |
| 35761 | ctaactttac ctagtctcag acaagttagt ttgcctcttt cagcctcagt tttcctcatc |
| 35821 | ttcaaattgg agataacagt aggtaatagc acctagctct cttttttttt tttttgagat |
| 35881 | ggagtctcgc tctgtcgctc aggctggagt gcagtggcac aatctcggct cactgcaacc |
| 35941 | tctgcctcct gggttcaagc gattctcctg cctcagcctc ccgagtagct gggattacag |
| 36001 | gcacacgcta cgatgcctgg ctaatttttc tatttttagt agagacgggg tttcaccatg |
| 36061 | ctggccaggc tggtctcaaa ctcctgacct caagtgatct acccgcctcg gcctcccaaa |
| 36121 | gtgttacagg tgtgagccac cacacctggc tgtacctagc tcttaaagtt catgtgacaa |
| 36181 | ttaattgaga tgaactacat aatgtgctta gcacacagtg ctcaaataaa tcattaatga |
| 36241 | aggcaacagg aaaaagccaa aatgagtaga tctccggggt aaggcctgag ttggttcttc |
| 36301 | aaaaggacac tcttaccaaa gaaaggctac caagtatatc agtcaagtaa acatttactg |
| 36361 | tttgccacat tccaggtccc ttacctgaaa ctgtgaccta aaagcaggaa taattcacgg |
| 36421 | cactgagcta ttttgaaaca gaaccaagct tgcttattgc acaagtaact aaaataggac |
| 36481 | atcaaaatat ttttcttgtt tttatttttt tgagacagtc ttgctctgac acccaggttg |
| 36541 | gagtgcagtg gtatgattac agctcactac agcctcaccc tcccaggctc aggagatcct |
| 36601 | tccacttcag cctcccaaat agctgggacc acaggcatga gccaccatgc tgggctaRta |
| 36661 | tttttaatta tttttgtaaa gatggggtct ccctatgttg cccaggctag tctcaaactc |
| 36721 | ctgggctcaa gtgatcctcc caccttggcc tcccaaaagt gctgagatta caggcatgag |
| 36781 | gcactgcatg tggcccagaa aatatttctg aatggaactt gaaaaaatgg atattttggt |
| 36841 | ctatacttca ccagccccac tgagcactcc agagccagat gtgaagggaa gagggtagcc |
| 36901 | agggtatccc ataactctct ttctgtatcc tgtcttttcc ccaccaggga gtaggaaaac |
| 36961 | attctatcct gaaaacaaga ttatttcacc tctaaacagt gacttttaat tattttgatc |
| 37021 | tcaacccaca atatgaaaaa cacatcatat tgtgtctgaa tacaaactca aaactgaaac |
| 37081 | aaaagtttca tcaattgaga cttgaaatgt gtgatacatt ctgggttttt ttttttttgt |
| 37141 | attctatttt ttttttaaat gttgatcaca attctctata aacggatttt aaaacctatt |
| 37201 | gacaggccac aaccagctgt tcgaaaaaaa gtactttgaa atatcacatg ggggaggaaa |
| 37261 | cgcagagatc atctagtcca atcccttcac ttcacagggg gtaaactgag tctcagaaga |
| 37321 | tcaagtcact tgcccaaggc cacgcagcta gtaagttgct gtggttggat ttgaccgtaa |
| 37381 | taaccatcgg ctactgaatg cccatcatgt gtcagccatt tgctgagttt tttctttttt |
| 37441 | tttctttttg agacagagtc ttgctctgtc gccaggctgg agtgcagtgg cgcgatgtcg |
| 37501 | gctcactgca acctctgcct cccgggttca agcgattctc ctgcttcagc ctcccgagta |
| 37561 | gctgggacta caggctcgcg ccaccacgcc tagctaattt ttgtattttt agtagagacg |
| 37621 | gggtttcatc atgttggcta ggatggtctc gatcttttga cctcgtgatc cgcccgcctc |
| 37681 | ggcctcccaa agtgctggga ttacaggcgt gagccaccgS gcccggcctg ctgagttctt |
| 37741 | taattgcatt atttcattta atactcactg tgatcctatg aaacagatac tattctttcc |
| 37801 | accttacaga cggggaaacg gagtctcaga ataggggaaa tgacttaagg tgtcacaatg |
| 37861 | gccagtagca gacccaggag tagtctttag gtctggccgt gcacaaagcc ccatgctctc |
| 37921 | aagctgtaac cccgctatca agaaagtggt tatgcaagtg ggcctgtgag ctctcaacgt |
| 37981 | ctaagccgtg cgcatgtgag acagcgataa gctcgcgttc tccagtctcc agggccttat |
| 38041 | cattcctcac gctttccttc atcggctcgc aaagaaacgt ggcgatcaag gaagacagcg |
| 38101 | aaggctccat cctcgtcggg ggaggataat tcgaatctca gcctgtgagt ttactcagca |
| 38161 | cacctgttta atctcttggt ccttcagttt ccacacctag gaagctggaa cagcagcagc |
| 38221 | atccagcgcc aggactggag tcatatacga actctggcac ctgggaggcg cgcgtcctac |
| 38281 | gtcagttccg ctcgacccca cccYccccgc ccccccgcct tctccccacg cgcatgcgcc |
| 38341 | gcgcagcggc gctttttacg acgcggcagg cgcggcgcgc ttagttgccg gagctgaacg |
| 38401 | gcgcggagct ggtctgaggc gagccgagcc gagcgagcgc ggcggtgggg ccgagaggac |
| 38461 | gcgcaggtgg cggcgttgcc atgtcgcacg gtcacagcca cggcgggggt ggctgccgct |
| 38521 | gcgccgccga acgggaggag ccgcccgagc agcgcggcct ggcctacggc ctgtacctgc |
| 38581 | gcatcgacct ggagcggctg caatgcctta acgagagccg cgagggcagc ggccgcggcg |
| 38641 | tcttcaagcc gtgggaggag cggaccgacc gctccaaggt gggccgcctg gggcttgggg |
| 38701 | cgggcggggc agggtgcgtg tggggcctcg ggccgtcggt ctactcattt ctgatgttaa |
| 38761 | gaataacgac agcctggttg ccaagcgctt ttccgtgcct gtctcctgcc agctctttgg |
| 38821 | gaggcgagca gcgttgtctc cccattttgc atatagggaa actgaggcct agagggaacg |
| 38881 | gggacagttt tgtcaagcta cacagcctaa cgctctattc cagggctgtg tgggctagag |
| 38941 | aggatggtcg tgagggccgt tggaccattg gtgctaatgg ctgcctcatc atattacctg |
| 39001 | gtgaagaYga gcctagggag atgaaaggtg gttcccatat agagcaaatg ttctttacat |
| 39061 | tgtggggtcg caggcccctt tgctatctgt ttgaaagcag tggaccttct tctcagaaaa |
| 39121 | atgcacataa acacctgcct ttcagttcac acttcagaag gctcttcgac cctggaaatt |
| 39181 | cattcatggg ctttttgtta agaaccttcc agttgggtgg cctgtggctg ttatgacact |
| 39241 | ggataagact ggttagcttc aagttaataa gtcttggaaa accaagggag agtgactctt |
| 39301 | cccattgcac cccagttgta gagcagggtg ggagatgcct gaaattgtag gctctcagca |
| 39361 | aatatttgtt gctttctcct cccccctccc catcccctcc agtttgttga aagtgatgca |
| 39421 | gatgaagagc ttctgtttaa tattccgtaa gtatctcctt ggtgcctacc tcaggctaaa |
| 39481 | taggctgtac ccagttgcct cagatggatt cattcactgg tgtcaagagc acacatttgc |
| 39541 | ttctagaaat gggataaatt actttggtgg gtttgaggag gaaagcatca tagctttctt |
| 39601 | ggtgcagact ggaaacagat gtatttgtag aacaataact gagaaattga ggtagatttc |
| 39661 | ttagaattgt agaatgctag aggtagacag gatcttagaa attatctagt acaagttgac |
| 39721 | agatgagtaa actgaagccc atacagggga agggtcttgc ccagggtcac acaactaaac |
| 39781 | aggtattcaa actcaggttt gacaaccttc tcttttgcat tctggcagat ttacgggcaa |
| 39841 | tgtcaagctc aaaggcatca ttataatggg agaggatgat gactcacacc cctctgagat |
| 39901 | gagactgtaa gtggcaaagg cttaggccct caaaggagct cctaattctc tttttctggt |
| 39961 | aacattttat tccagaatag tatcNtttNc ttcttaccgg gtttaatatt tcccctttta |
| 40021 | aaatatcttg gtcctgttct cattctgcat tggaatacat atatttcttc ctgaattaat |
| 40081 | gactttttta gattaagcat ctttaggccc aacagcgtgt ctcatatttc aaagtgctta |
| 40141 | ctatgacatg attcattcag agagcattgt gtaaatgtca cttgttgatg actctgcagt |
| 40201 | tttcttaata actagctgca gatctcatat gatctttatg gattctaggg gttagcttca |
| 40261 | acatttttcc ttgctatttc atacagtaat gatcttatgt gttttgttga catggaattg |
| 40321 | ctccacctga ctttgcgtgg catttagtta gcaaagttgt gtatacctct tgcaacactg |
| 40381 | aatcttttgc caccagagct agtacccttt ttgacaactt accttattcc agaacagcct |
| 40441 | tcctaaaaga aggggaaaac tgctttctga gtgctggaga aacatataag aagaacttta |
| 40501 | catttaaaaa attcagtccg ggtgcagtag cttatctcac aaaaccaata aaagatgata |
| 40561 | tataggctgg gcacagtggc ttacacttgt aatcccagca ctttgggagg ctgaggcggg |
| 40621 | cggatcacct gaggttcgag accagcctgg ccaacctggt aaaaccccgt ctctactaaa |
| 40681 | aaWacaaaaa ttagctgggc ctggtggtgc gtgcctgtaa tcccagctac tagggagggt |
| 40741 | gaggcaggag aattgcttga acctgggagg cggaggttgc agcgagccga gatctcgcca |
| 40801 | ctgcactcca gcctggatga cagaatgaga ccccatctca aaagtaataa taaaaacaaa |
| 40861 | taaataaaaa attcaaatcc taaaggccga gcatggtggt tcacacctgt aatcccagca |
| 40921 | ctttgggatg ccgagactgg ttaggagttc aagaccagcc tgaccaagat gctgaaaccc |
| 40981 | cgtctctact aaaaatacaa aaattagccg ggcatggtgg cacacgcctg taatcccagc |
| 41041 | tacttgggag gctgaggcag gagaatcgct tgaacccagg tggcagaggt tgcagtgagc |
| 41101 | caggatcgtg ccactgcact ccagtctggg tgacagagca agactccatc tcaaaagaaa |
| 41161 | aaaataattc aaatcccagc actttgggag gccgaggcgg gcggatcact tgaggccagg |
| 41221 | agttcaagat catcctggtc aacatggcga aaccctgtct ctactaaaaa tacaaaaatt |
| 41281 | agctgggtgc ggtggcacag gcctgtaatc ctagctactg gagaagctga agcatgagaa |
| 41341 | tcacttgaac cgcggaggtg caggttgcag tgagccaaga ttgcaccact gcattccagc |
| 41401 | ctgggcaaca gagtgagact ctgtctcaaa aaaaaaaaaa aaaattcatc tgaaaaattg |
| 41461 | attaaaggac ttccagagat ttaagatata ttatagttga gtattagaaa catgaatttg |
| 41521 | aaagcaaatc catgtattta atcattttaa ggaaataata cctttgatgc ttttggagac |
| 41581 | tcatttactt ggaagttgat ggactgacct gagaatatta ggcatccatg ctgcattgtg |
| 41641 | atttagagtg tgggatctgg agtaaactgg gttcaaataa tggctgtacc acttgttagc |
| 41701 | ttcatgactt tgagacctta tcttctctat gcctggattt tctatatcta acatgggaat |
| 41761 | ggtaacagtg catacatacc tcataagatt gtggagaggc ttaaatgaga taaaacatgt |
| 41821 | aaagagctta gattaggtct ggaacacagt aagtgtccaa ttttgggtga acagatacga |
| 41881 | atacatagcc ctcagttcta agcacactac atatattata ctttatcaaa tgtaagatgt |
| 41941 | cattgattgt aaaatgtacc attatattca attaatacag aaaaaatgct attgaactat |
| 42001 | gacatgtcat tgacacatcc agaaaaatta gattgagagt attaaatcag ggaagtttga |
| 42061 | acacagatga ggtgttacat gttactaaag aattaccatt cagccaggcg cagtggctta |
| 42121 | cacctataat cccagcactt tgggaagccg aggcaggYgg attacctgag gtcaggagtt |
| 42181 | caagaccagc ctggccaaca tggtgaaacc ctgtctctac taaaaacaca agaattagcc |
| 42241 | agtcatggtg gcagtcgcct gtcatcccag ctattcggga ggctgaggca ggagaatcac |
| 42301 | ttgaacccgg gaggcggagg ttgcagtgag ccaagattac tccactgcac tccaacatgt |
| 42361 | gtgacagtgc aagactctgt ctcaaaagaa aaaaaaaaaa gaattaccac tgggctgggc |
| 42421 | acagtggctc atgcctcaat cccagcactt tggggaggcc gaggcgaaag gatcacttga |
| 42481 | gcctaggaat taaaggttat ggtgaattaa gatcgtaaga tcatatactg gaccccagcc |
| 42541 | tgggcaacag agcaaggctt aaaaaaaaat tatttatata gtttgagata gggccttact |
| 42601 | ttgtcaccta ggctggagtg cagtagtgtg atcatagctc actgcagcct cgacctcctc |
| 42661 | agttcaagca atcctcccac cgcagcctcc caagcagctg ggaatacaga cgtgtgccac |
| 42721 | cacgcccagc taatttatgt atttttttgc agagatgggg ttttgttatg ttgcccaggc |
| 42781 | tggtcttgaa ctcctgggat caagcaggct gcccacctca gcctcccagt gtgctgagat |
| 42841 | tacaggagct agccactgca cccagccaaa aaaaaattta aaaaagtgaa ttgctgttaa |
| 42901 | ttttgttatc tgttgatatt gtcaattatt gtaaaatggt attaaatata tgtattttat |
| 42961 | tttattttat tttatttttg agacagagtc tcactgtgtc gcccaggctg gagtgcagtg |
| 43021 | gcgcgatctc ggctcactgc aagctccgcc tcctgggttc acgccattct cccatctcag |
| 43081 | cctcctgagt agcggggact acaggtgccc gccaccatgt ctggctaatt ttttgtattt |
| 43141 | tttagtagag atagagtttc actgtgttag caaggatggt ctcgatctcc tgaccttgtg |
| 43201 | atccacccac ctcagcctcc cgaagtgctg ggatcacagg cacatgtgtg tatatatata |
| 43261 | tgtatatcta catatatacg catatatata tacacatata tgtgtatata tacatatata |
| 43321 | cgcatatata tatacacata tatgtgtata tatacatata tacgcatata tacacatata |
| 43381 | tatgcgtata tatacgtgtg tgtgtgtgtg tgtgtgtgta ttttttgttt ttttaagacg |
| 43441 | gcgttctgct ctgttgccta ggcttgagtg cagtggccca atcttggctc actgcaacct |
| 43501 | ccgcctcctg gggtcaagcg attctcttgc ttcagcctcc tgagtagctg ggattacagg |
| 43561 | tacacaccac caggcctggc taattttttt tttattttta gtagagacag ggtttcgcca |
| 43621 | tgttgaccag gcagctctcg aactcctgac ctcaagtgat ccgcctgcct ccgcctccca |
| 43681 | aagtgctggg attacaggcg tgagccacca cgcctggccc atactggaat atttataggt |
| 43741 | gaaatgatgt gtctgggatt gctttaaaat attgagctta agcaaaatca ggctgatagg |
| 43801 | caagatcaac aaaatgttga tcatcgaaga tgagtgaaaa gtatgtggag attcattatg |
| 43861 | taatctctgc ttttgtgtaa gtttgaactt ttccatagta aagttttttc aatatatgcg |
| 43921 | ttacttagaa tcgattcaat ttcatctcca taccaaccgt gtggaatatg tactattacc |
| 43981 | attcccattt tacagatgag gaaacagagg ttaaatcagg tgcccaaatt tactcagctg |
| 44041 | gtaagtgaca gagccaggct tgaatcctag aaagtctagc tctagatttt aactacagcg |
| 44101 | ctatactgct tctcattatg ccgctcataa atgtttacta ttattattat tctgtggtgc |
| 44161 | atcattctta gaaacacttt gaagtaaaca tttgctttct tttttttttt tttttttttt |
| 44221 | tttgagacgg agtctcgctc tgttgcccag gctggagtgc agtggcgcca tctcggctca |
| 44281 | ctccaagttc cgcttcccag gttcatgcca ttctcctgcc tcagcctccc aagtagctgg |
| 44341 | gactacaggc gcctgccacc acgcccggSt aattttttgt atttttagta gagacggggt |
| 44401 | ttcaccgtgt tagccaggat ggtctccatc tcctgacctc atgatctgcc cgcctcggcc |
| 44461 | tcccaaagtg ctgggattac aggtgtgagc cactgcgccc ggSggctttc ttaaattaca |
| 44521 | tgcattttgc cccagctaat atcgaggcct cttctgtgtg ggtgtgtcaa taaaacaccc |
| 44581 | tggaattggt ggggacatgt ggcttagtct ttgggaattt gtgggttata gagacagctg |
| 44641 | ctttttagat tactctttga aaaacatttt cttctctagc agtttacttt ttatttattc |
| 44701 | tgagacagag tctctctgtc acccaggctg gagtacagtg gcgtgatctt ggctcattgc |
| 44761 | accctctcta gcggtttgga tgtgagattg cagtcttttg tgatctttca aaatggaaat |
| 44821 | cctcatgtta taaatgtttt aagaactgta aggtgactga gtgtatggcg tggtttttgg |
| 44881 | acacaaaaaa atgcttgagc attgggttac ctgatctttt atctcttaac tgtgtgtaat |
| 44941 | atgtttctgc caaagtatgt atttttccca tgagtaagct gttttattta ataacatcct |
| 45001 | cagtttggtc tgaatgtgct gagttgttta ttttctccat gcatttgctg catgtgacct |
| 45061 | agtttgagcc gtctctttgt gtattgcttc tcatgtgata ttaacccact caaagatgcc |
| 45121 | atggggttat gttgcagatc tcagctcttt ttttgttgga ttaaagatat ttgtaggcta |
| 45181 | gacgcggtgg ctcatgcctg taattccagc actttcggag accgacgtag gcggatcacc |
| 45241 | tgaggttggg agttagagac cagcattgcc aacatggtga aaccctgtct ttactaaaag |
| 45301 | taccaaaatt tgccgggcat ggtggcgtac gcctgtagtc ccagctactc aggaggctga |
| 45361 | ggcatgagag tcacttgaac cYgggaggtg gaggttgcag tgagctgaga ttgtaccatt |
| 45421 | gcactccagc ctgggtgaca cagtgagact ctgtctcaaa aaaaaaaaaa aaaaagatat |
| 45481 | ttggagatac ttgtataaaa tgtcatttct aaaatatgac ccccaaattc agtctcctat |
| 45541 | tgatagaacc aaagccattt aagcagtcag tagaaaattt tgaaaacctg aaacgtgata |
| 45601 | atgcacattt cttgactttt ctttcctttc ctttctcagg tacaagaata ttccacagat |
| 45661 | gtcctttgat gatacagaaa gggagccaga tcagaccttt agtctgaacc gggatcttac |
| 45721 | aggagaatta gagtatgcta caaagtaagc actgggcctg tcttccattc tctatggctt |
| 45781 | cttatagggc ttgccattta tttttggcca gtctcctgct ctcttagcat tggaaatgag |
| 45841 | ttaaaacagt ggaggagact tgatctttgg cttggtcagt ttttggcaga gattcagtgc |
| 45901 | aagccacaga cttgttaact tgcaagcaat tggcagatct gaatgaactc ctagggtggt |
| 45961 | tttaggaaag atgtgtctcc cattagttta ggttaacact aaatgtgctt tagaatccct |
| 46021 | gctgggttat gcagcatttc tcattcctgt tccttgaggg caggagcagt gtttatccct |
| 46081 | actgccttgg acaggatctg gcaggtgctc agtaaatggt cattgaatta attgcctgtc |
| 46141 | caaaggagaa ggaagaactc aggagctctg Mctttgcctg gcttttacaa gctggagctg |
| 46201 | aattgttgtt tcacatggtg gtgagatact cattgaacta taattcatac cttctttccc |
| 46261 | tattcctgtc atcctctaga atttctcgtt tttcaaatgt ctatcatctc tcaattcata |
| 46321 | tttcaaaaaa cttcggagca gatacgacaa aggtctttta tattggcctg agaggagagt |
| 46381 | ggactgaggt aagatggggt tggaaaggtt ttcccctcat gtctacatat ctgcacactg |
| 46441 | tggtgatggg agcttccagg gaagggaaaa gagcaagtgt cataattact cccagtttat |
| 46501 | tatttttctt ggttttatca gtataaactt ttcgtctcat ttgagaaact acaggtattt |
| 46561 | ttaagggggt tgaaactgtt atttttatat aaataatata tataatatat atatatatat |
| 46621 | ctcagctcac tgcgacctcc gcctcccagg ttcaagtgat tctcctgcct cagcccccca |
| 46681 | agtagctggg attacaggtg cccactccca cgcctggcta atttttttgt atttttagta |
| 46741 | gagacagggt ttcaccatgt tggccaggct ggtctcgaac tcctgacctc aggtgatctg |
| 46801 | cccatctcag cctcccaaaa tgttgggatt acaggtgtga gccaccttgc ccagcctgaa |
| 46861 | attgtcattt atattaattc acagttttct actttcaaat gctttcattg agtatttttg |
| 46921 | ataatctttt ctattcatga accagggata agataatacc atagacaagt tcctattctc |
| 46981 | atggtacccc cattatagta gggagactga atcttcaaag ttacagggtg aatcaatgat |
| 47041 | aatgatcttt gcagctttct ggagttaaaa agtatcaaaa ttgggagata ttagatgatg |
| 47101 | acatctaagt attaaaataa ggagatatta aatgatgact cctagaaatg aacctgaata |
| 47161 | aggactaccg caatgtgtgt ggtgtgggaa aggacagttc ttttaatggc tggctgaccc |
| 47221 | agcctcaatt ttcttgcagc ttcgccgaca cgaggtgacc atctgcaatt acgaaKcatc |
| 47281 | tgccaaccca gcagaccaYa gggtccatca ggttacccca cagacacact ttatttccta |
| 47341 | agggctggcc aaggctccca tagaggcgct gtgtcagtga agatgtacga ctacctgttg |
| 47401 | ggaaggacaa agggatgagg ctccagagag agttggctgc cacagcctct gccaagcttt |
| 47461 | gtctttgggg cttgctgcag aaacctggcc tacggaagat acgacaccac tgggagggtt |
| 47521 | gtgtaggtgc caggggacca tcgtggttct ctagggcgct gtggaaattg ggtcttgggc |
| 47581 | tgggtggcat ctggcagtca tggRtaacac ttgcttttcc agttaatgtg gccatgtgat |
| 47641 | tccaagtgtc atgttgcttt gtggcaagat tgttgtgtga cttgtttttt tgtttttgtt |
| 47701 | tttgtttttt taaaggaaac tatttgtggg ctataggaaa ctttctgatg cctccggatt |
| 47761 | gtgttagtag tagccatcag gagggtctcc aactaaaaca cttgttcctg cttgctcctt |
| 47821 | tcccctctca ttgttcagca ttcttgtcaa gttgcccagc ttggagttgt ctgtcacgca |
| 47881 | catgtgtcct gtggttatag ctagaaggac aggagtctcc tgctgatgcg tgatagctta |
| 47941 | agcttgggga gaaggtcttt tccactgcct agctaagcag tctggggaga gcatggggat |
| 48001 | catttctatg tgtgtgggta atctggtcag taagattgag acttagttaa gattcccctt |
| 48061 | ggaaattcct taatgtttat tagcttctaa ctagtgttgt aagtccgatg ccagaatttg |
| 48121 | gagatttgag ttcttctttt catggctttt attcactgtg actaataagc ttcctaataa |
| 48181 | atccttgcca gacttaatgt ttgtattttc ttttgagttc ttgatagcca ctgatgcaac |
| 48241 | tttcatttca tggggaactg tactgaactt tgaaattttt tgttgttgtt ttggactagg |
| 48301 | tagattatag gaagtgccac aattaaatca agactggagc cgggcgcggt ggctcacgcc |
| 48361 | tgtaatccca gcactttggg aggccgaggt gggcggatca cgaggtcagg agatcgagac |
| 48421 | cacggtgaaa ccgtctctac caaaaataca aaaaattagc cRggtgcggt ggcgggagcc |
| 48481 | tgtagtccca gctactcagg aggctgaggc aggagaatgg catgaacccg ggaagcggag |
| 48541 | cttgcagtga cctgagattg caccactgca ctccagcctg ggtgacagag tgagactccg |
| 48601 | tctcaaaaaa aaaaaaaaaa aaaatcaaga ctggatcagt ttctccagga atggtcaagc |
| 48661 | ttaattttgt acagtaggcc aaaaacactc ctacacccat atatagtaaa gaagaaggtg |
| 48721 | gggttgaaaa cctgtatttc acagaaatta actgggcatg gtggcgtgca cctgtagtcc |
| 48781 | cagccacttg ggaggctgag gtgggaggat cactggagcc caggaggtcg aggctacagt |
| 48841 | aagccatgat tgcgccactg caccccagcc tgaggcaaca gagtgagacc ttgtctcaaa |
| 48901 | acaacaacaa caaaacccta tatttcattt tgcacaaaag gaagtgattt gtagaatgac |
| 48961 | atttacaaaa ataagggaga acagattttg gagctcatca gaagaaatga gggtgaggaa |
| 49021 | agtaattgag cagctcatgt ggtctccagg tagaccaaag actttggact taaattttga |
| 49081 | aagtcagttt agcaattgaa gagaatgtaa gtatctaaaa aggggatttg atatggtcag |
| 49141 | tattctctcc cttgcagaaa acaggaacag gtaataaagg gaattagtgt tgaatctgat |
| 49201 | ccttttggga gaattcctct gaggcattgg cccatgctgt tgtttggagg atatagccac |
| 49261 | tgtttctcca gcctgtccta ggaaaggtct gctaccacca aaccttgttg gccctcaaaa |
| 49321 | tttctaaaat gcagcctgaa ataaaccaga atgcaaatgc tgttcttgaa aagcatgaag |
| 49381 | ggtcctattc actcagccac agcctcttta tttcagttgg atcatttttt ggagttaaaa |
| 49441 | gaagggctat gtttaaattt ggagtagggg gaaaaataaa gttaaaaatc ttagagcaga |
| 49501 | ctagcccttc actaattcca ctaattaaag cattaatcac agctcctggg actgagcctc |
| 49561 | tgaatttgaa gcttcactct gaagaatttg ttttaagtgt catggtgtct caggtgatcg |
| 49621 | aggttgcagg aggaactgtt cattcttcca taagtgggga cattaccctc cattttcttt |
| 49681 | tgacattcag tcctgggtcc ttggcttgtg gtggtcccat aggctcaaag gtttcctgcc |
| 49741 | ccgaatcctg gtattgtagg ctccctgacc tgttgagtac ttaggaccaa agtgcctctg |
| 49801 | ggcctggggg aaagtgcttt gttgagcttc caactttgta ctgttttctt tttcttttct |
| 49861 | ttcttttttt tttttttttt tttttgagac agagtctcgc tttgttgccc aggctggagt |
| 49921 | gtagtggcac tatcttggct cactgcaagc tccgcctccc gggttcacgc cattctcctg |
| 49981 | cctcagcctc cccccgagta gctaggacta caggcgcccg ccacagcgcc aggctaattt |
| 50041 | tttgtatttt tagtagagac agggtttcac cgtgttagcc aggatggtct cgatctcctg |
| 50101 | acctcgtgat ccgcccgcct cagcctccca aagtgctggg attacaggag tgagccaccg |
| 50161 | cgcctggctg tactgttttc tttctgttca aagaaaacac tagtataggt taggctggga |
| 50221 | gagtgagtcc taagcggctg agggtcaagt gtgactcatt tcctatttgc agatccccac |
| 50281 | acattctcag ggacatgctc cttcaactca gtttgcctgc cttctgactt ttctgtggtc |
| 50341 | cctgatacct atttccactt cagcttgatg gcagcgtttt gtgcactgtc tgcatgacat |
| 50401 | tgcttgtgaa gggcagcgct aacactgcca gtgcctgtcc agctctcctg atacggcctt |
| 50461 | tgtatttttc ctaggtcctt ttttgcgtga ttaacacctc acgctgtgat tttactgtat |
| 50521 | aaaccatgtt cacacaacct catttaagcc taccaacaaa gctgtgaagg aggcagggtg |
| 50581 | gttattacca ttttatatat caggaagcag atccagagag gcagaattta aatggaatcc |
| 50641 | cgaggttgct gtttacatgc cttccttgag cctcagtttc cccatcttca aggctggaga |
| 50701 | actagatgat ctgtaaggtc tcttcggttc agagtgtgtc attccattca tgatcctgga |
| 50761 | atagggcatg aaagccagtg gcagaMaccc gaggccttaa gaggtgtgtc ccatgaggat |
| 50821 | gggaaggtgg taaagtttat gcaaacaata gtgtgaggga tgcctctcct tccggtcggc |
| 50881 | cctcctccag gaggcgcatg ggtggtggta gcaagtggga gtcttgggtg ccctgtctcg |
| 50941 | aatctacaag gtgggagcgg gtgagaaccc agcgactgcg cggctgaatg acagggagta |
| 51001 | gccccgccgc ctccaacatg gcagcttcta tccagcgaag ccgggtcccc gcttccKggt |
| 51061 | tccgtactgt cggccaggct acttccggca gcgcgatggc tgggttcccg ggactcgaac |
| 51121 | ggaagttccg gcgggggcgg ccgaggggga agagtgtgtc tgcgggagaa agaggagaat |
| 51181 | cgcccaagcg gcctcggaag tcccagggag tggaggtacc cagccggcgg ctgcggagga |
| 51241 | tgggcgtgta gggggaggtc ggcctcggga gggtcccgga gagcggggag ggggaaccgt |
| 51301 | gatggggggR atgggggtgg ggagtgaggt gcgaaggcgc tgtccagtct tggggacttc |
| 51361 | gggagtggag cggggctagg caagggggtg tctgagtata aagagacaga cttcagggtc |
| 51421 | ggaaatagaa gagcccattg tggggcgggg gcttccccag agtcaaaggt acaaagcttt |
| 51481 | attgtgggca ggtgggagtc ctgggtcagg gtagaaaatt tcactgggta gggtgacctt |
| 51541 | tggtcagaac agaagttccc attgtgggga gcgggagatc ctgggtcagg gtggaagttc |
| 51601 | tcaaggtgta tagagagaga caggtcaggg ggaaggcacc cctccgagga gggagcctgg |
| 51661 | gcgggaggtg caagtcccca cggtggagaa ggaagggaat cgcagctcag ggcaggaggt |
| 51721 | gcattcccca tgggaagagg aaccccgggt cacaggtgaa cgatcccatt gtggggagct |
| 51781 | gccctcaggg gtcagagatg gaaggtgagg ccagttggga ggggtgggca ggcacttgag |
| 51841 | gcaggtagtg gaagcttccg gcgagtttgg aggacgccga ggatggggag gggccagcca |
| 51901 | gcctgacatt cttttgtgtg tttgggagtc ccagagcgtg gtcaagccag ggctcgtttg |
| 51961 | cagtcagtgg cagcagcagc aggtagctcc gtttaNgggg ggcgtggagc accagtttgg |
| 52021 | gggtgggtgg agttcttacg ctttctgggg acatgggcag ccccctgggg gtctgggaat |
| 52081 | ggatgggcca gaaactgctg tgaaggctgg atgggggctg ggtgggggga ggttgagatg |
| 52141 | ggggttggag gcggcactga gtaggggatg gaagctcctt tccctggcac ttttctctcc |
| 52201 | atttcccata gggcccggag ttggggaggc agggacccag ggatgattgg agaggagggg |
| 52261 | gaaggcactc acccctgagg agttagaggg ggaatgaagc aggcgtgccg ctgcctcccc |
| 52321 | tctgccccct gccctacagt ccctatgccc tgcctctgcc tgtctccagc aagctcaggt |
| 52381 | cctgtggttg ggccagaacc agagacacat gttctgggcc actagggagg ctgtctctgc |
| 52441 | ctttcagccc tggaactgag gcagacttct tctcctgtgc cctgggactt cttgcttgac |
| 52501 | tattctttgt gcccaggaca gaacctgggt gctgtgtggg gctgaKggcc tggtggcctc |
| 52561 | ttgggccagg agtgtgtcct gtgcctggtt ttgctcttga cYgccgcccc gatgtatgca |
| 52621 | ggcccccgcc gtggagccgt gtggtgtatg tgtggtaaca ccatgtctgt gcccctgctc |
| 52681 | accgatgctg ccaccgtgtc tggagctgag cgggaaacgg ccgcRgtaag ggtccccttt |
| 52741 | cacccacggc cagacactgt gggagcagct ggaggaccgg ctggaggggg tgtgggaaaa |
| 52801 | tggaggtggc agtgatgagc agggagtatc ctgaggccag caggctcggg gtagtaaMct |
| 52861 | gtttggctgc tacctgggtt cccccaggtc cttgggggtg ggggaggggt gggcagaagt |
| 52921 | tggaggacct agggKaagcc ttcctgtYtc cccatttccc atccttttcc aggttatttt |
| 52981 | tttacatgga cttggagaca cagggtaagt gactgaggag gggtgctcct taaggagggg |
| 53041 | tcctggccca gcagcggact ggagaggcct tctcttccta ccacatcgga gccttttctc |
| 53101 | cYgtccctcc tacaggcaca gctgggctga cgccctctcc accatccggc tccctcacgt |
| 53161 | caagtacatc tgtccccatg cgtgagtgtc accccagcaa gggaggggct gaggctgggg |
| 53221 | atcatctggg ggtggtcctg tcatcccagg cctgttctct cctgtcagtt gcatcctggg |
| 53281 | gcttgggctc agggcagtca gaagtggcat tttcagcctg agctcctgtg gagcccccag |
| 53341 | gagtggggtc cagcaccagc ccccagccct agggcctcct agacctgagc tctcctagaa |
| 53401 | cccacacctc cctttgcccc ttccccaagt actcaccgac tacctgggac cccgtcacac |
| 53461 | caagcgctgg gctctgcttc tccattactg gcgctttgcc ccaaccacag cctccagccc |
| 53521 | cacgtggcag gttgcctggc aacaccttaa acacaggccc tgcctgctgg gaggggccac |
| 53581 | caggggcggc gcggccccac tccgggctct gagctctggc ctcctcattc ctcctgagca |
| 53641 | tgatgggccc tctggctctc tttccccagg cctaggatcc ctgtgaccct caacatgaag |
| 53701 | atggtgatgc cctcctggtg agtttgggag tgggggtggg cagggggacg aggacacttg |
| 53761 | ggctgaggga gccacagggg gctggctggg gttttctata gctgccagtg cctctactcc |
| 53821 | caaggtttga cctgatgggg ctgagtccag atgccccaga ggacgaggct ggcatcaaga |
| 53881 | aggcagcaga gaacagtaag accccagccc ctcctccaca tcctccttcc cctgcccccc |
| 53941 | aggaaagcgc tgggcggttc ctcagcctag ctcccttatt gggccccttg gcagcttccc |
| 54001 | tctacccact catgccccct cccccagtca aggccttgat tgagcatgaa atgaagaacg |
| 54061 | ggatccctgc caatcgaatc gtcctgggag gcttttcaca ggtgagggga gaggggtggg |
| 54121 | ggggtagggg gtaggggtgg ccggtgagtg agctgtgccM tcatgacccc tctctctcct |
| 54181 | ccctccaggg cggggccctg tccctctaca cggccctcac ctgcccccac cctctggctg |
| 54241 | gcatcgtggc gttgagctgc tggctgcctc tgcaccgggc cttcccccag gtgaatgtcc |
| 54301 | ccactgaccc ccccgccctt tgtgtctgca tcctcgtggc ttggggactg ctgcagcact |
| 54361 | agctttgccc tgagtcctgt ccggcctcca ggcagctaat ggcagtgcca aggacctggc |
| 54421 | catactccag tgccatgggg agctggaccc catggtgccc gtacggtttg gggccctgac |
| 54481 | ggctgagaag ctccggtctg ttgtcacacc tgccagggtc cagttcaaga catacccggg |
| 54541 | tgtcatgcac agctcctgtc ctcaggtcag tggggggacc atttcccact cccacttctc |
| 54601 | tgcctccagc caggtgcctc tcgtgatccc ctcttaatcc cctcaggaga tggcagctgt |
| 54661 | gaaggaattt cttgagaagc tgctgcctcc tgtctaacta gtcgctggcc ccagtgcagt |
| 54721 | accccagctc atgggggact cagcaagcaa gcgtggcacc atcttggatc tgagccggtc |
| 54781 | gagcccctgt cccYaccYtt cctgacctgt ccttttccca caggcctctg ggggcaggtg |
| 54841 | gcaaggcctg gcYgggcctt ccttcctggc cttagccacc tggctctgtc tgcagcaggg |
| 54901 | gcaggctgct ttcttatcca tttccctgga ggcgggcccc cctggcagca gtattggagg |
| 54961 | ggctacaggc agctggagaa aggggcccag ccgctgaccc actcactcag gacctcactc |
| 55021 | actagccccg ctttgggccc cctcctgtga cctcagggtt tggcccatgg ggccccccca |
| 55081 | ggcccctgcc ccaactgatt ctgcccagat aatcgtgtct cctgcctcca ctcagctgct |
| 55141 | tctcagtcat gaatgtggcc atggccccgg ggtccccttg ctgctgtggg ctccctgtcc |
| 55201 | ctgggcagga gtgctggtga ggaggtggag ccttttgagg ggggccttcc ctcagctgtt |
| 55261 | tccccacact ggggggctgg gccctgcctc cccgttaccc tccttccctg caggcctgga |
| 55321 | gcctgtaggg ctggactgag gttcaggtct ccccccagct gtctcacccc cactttgtcc |
| 55381 | ccactctaga gcagggaggc agtgggggag gagttgtgtc tYgtcttctg tctccatgtg |
| 55441 | gtttttgggt gtttttcttS ttgtgtcctg gattccgata aaattaaaga aattgcttcc |
| 55501 | tcaacgctca ggcctggctg attctattcc aggggtggat agtgaccatc tgtgagaacc |
| 55561 | tgccccagtg taagaaaact tgtttttatt tttaaatact ttggaaagct ctttcagagc |
| 55621 | aatataaatg agtgcctggg aggaKgaggt tttgtgccaR agccttgccc cctcactcac |
| 55681 | tcttgggggt cctgatgaac tcttggaccc tgtggaagat aagagttaRa gacctMggcc |
| 55741 | tcctggtcag tggagccctt ggcctcatgc ctggtgggct aagcgggccc agctggggtt |
| 55801 | tgaggtaggg gaggccttgg Yttggcccca gcagctccag ggccctgagt tcctgccaga |
| 55861 | ggctggagag caggcagctg ctgcttttcc tggtccttgg taggggaggg tcctcaggct |
| 55921 | tgcgtgccaa agcctgaagg attctgcttc tgccagcgcc agagatcctM acctgcaaca |
| 55981 | cggcgaggtg tgtgctcagg gcccacggtg gaatgcagag cctcccccac cccacagccc |
| 56041 | gccctgggtg ggcatgccca gatctgattt agcccctccc tggcccctaa ctgcaggggt |
| 56101 | caggccagca ctcacacatc ctgtccagcc ctaaggctgc tgtccacccM agctcctctt |
| 56161 | gggccaRgct Kgggttggcg taacaggctg ccacatcacc ttcccgccgK gcMaccacct |
| 56221 | tgtacgggat ctgcaagaca ggaggtagtt ggagcttaKc tgagccggcc ctggcccagc |
| 56281 | tgctggccag gtctttaaga agcagaagtg cagagcagcg gctggcccgc aggcaccggg |
| 56341 | tgctaggcag gctgaggaga ctgggcagtg ccaggtaccc tccagagagg tgagtgggaa |
| 56401 | gggccaaagc tgctctgttg ctgagagctg ggtggggtga ggtgggtgag gtgggtgggg |
| 56461 | Ygggggggcc taccttcttY ccagaRgcct tctccataRc ctggaccatY tgcagcactg |
| 56521 | aatagcctgt gcccgtgccc agKttgtaga tctggcccac ggagaacagg gtttatggag |
| 56581 | cgggctggac tgaccacgcc tctgggccgc tctgcctgga tctggtccct cccctccctc |
| 56641 | acttctccct tctcttccta cccggcagcc acactgttct ttcagcttcc ttaaggctgc |
| 56701 | aatgtggccc ttggccagat ccacgacatg gatgtaatcc cggacacctg cagagaaggg |
| 56761 | agtgtgttgg atggggagtc tgttccccct gactctcctt cctggctccc actcctaggt |
| 56821 | ccccctSgtc ctaggctcac ctgtgScatc ctctgtgtMa tagtcattgS caaagacatt |
| 56881 | cagggcctcM cgtcgcccga tcgccaMctg gaggtggaga tcaggtcagt tcgtcccaga |
| 56941 | tcccaggcac cagctttaac cccagggcca ctcctctgtc ccttcccttt tgccttacct |
| 57001 | gggagacata aggcatgagg ttgttgggta tgccctgggg atcctcacca atgcagccag |
| 57061 | aggcatgggc acctgtgggg ttgaaatagc gcagcagcac tgcgttccaa gtctgtggga |
| 57121 | tgtgggtcag gtggtgaggc cagaggcaca ggcagcgtgt cccagctgaa cccagagccc |
| 57181 | tcctcatgcc tattctgttt ttttgagatg gagtctcact atggcccagg ctggagtgta |
| 57241 | gtggcctgat ttggctcact tcaacctctg ccttccaggt tcaagcaatt cttgtgcctc |
| 57301 | agcctcccga atagctggga ccacaggcac acaccaccat ggccggctaa tgtttgtatt |
| 57361 | tttagtagag atgggggttt taccatgttg tccaggctgg tcttgaactc ctgacctcag |
| 57421 | gtgatctgcc cacctgggcc tcccaaagtg ctgagattac aggtatgagc cactgcgccc |
| 57481 | ggccagttgt ctatttatta tctttagaaa caagcagggg atggggccct ccactgcaac |
| 57541 | tgcctgagcc ttagcttcct ccttagtggg ggtgttcgga atcccacccc tgggctcagg |
| 57601 | gtgggccctg aggagtccat cgatcagtgg agccagggca ctgtcaaggg ggccctcacc |
| 57661 | ttgtctgcct ggcacaggtc ccggatcatt tcctcgatga agaacttgga cttgccgtaa |
| 57721 | gggttggtac aaccacccgt ggggtgggcc tcatcaaggg gcaggtRctg ggggttcccg |
| 57781 | tacacagtgg ctgagctgct gaacaccagg ttcttcaccc cgtgggcctt catgatctgg |
| 57841 | ccgtggagag atggcatcag tgacctctgc ctcacacatt actcccaccc tgttactcct |
| 57901 | ccacaggaga atggggctga ggtgatgagc actctcattt acagatgggg aaactgagac |
| 57961 | tgggaggtaa agacatgagt ccaggatgat acagcttggg ctctgtgttt ggcactgcct |
| 58021 | gccaggctgg ggtccagctg gacaccctcc tagtgtctgt gccctgtccc atgcctctca |
| 58081 | cctccagaag ctggatggtc ccggtcaggt taactctgta ataatccaga ggcttctgca |
| 58141 | ccgactcgcc cacggccttg agccccgcaa agtggatgac cgccataaag ctgtactgca |
| 58201 | ggggtgacat ggccagaggt tgctctactg gttttagtcc ttggcaatgc cctcagcctg |
| 58261 | cctgcctgca ctcacctttt tttttttttt tgacagtctc gctctgttgt ccaggctgga |
| 58321 | gtacagtggt gccatctcag ctcaccgcaa cctccacctc ccaggctcaa gccatcctcc |
| 58381 | catgccagcc tcccgagtag ctgggaccac aggtatgggc taccatgccc agctaatttt |
| 58441 | tgtatttttc tgtagagatg gggtttcact gtgttgagca ggctggtctt gaacacctgg |
| 58501 | gctcaagtga tctcctcacc tcggccttcc aaagtgctgg aattacaggc gtgagccacc |
| 58561 | gtgcccagcc tgcacccacc tttttgaaga gacgctgtag ggctccctgg tccaaaatgt |
| 58621 | ccatctcctc aaactccaca gagcggcctg tcagctcctg gacccgccgc aggctctcag |
| 58681 | gcagggagcc ccctcctggt agggtacatg taggccacat catcacgaca tggggtgctg |
| 58741 | tgttcccagg gactagccag acacacattc cccScccggt ggtggaggtg gaacccaggg |
| 58801 | ttttgcttct gccatcccct caagtagccc cagccccact gccccgctca ccacggaagg |
| 58861 | cattatggaa gttatcgatg accacaggca agtagccagc ctccagcagc tccagcaccg |
| 58921 | tgtggctgcc aatgtagcca gccccacctg ttaccagcac cttctctgcc atggcacctg |
| 58981 | gcccaggata cagagtctca gaggtggctg aggctgcctg ctcagagctt ccttcccact |
| 59041 | ggaatcctgc cccctaagct cgctttggaa ggaggcggca gtgtgcccta ggtaccagac |
| 59101 | tggctcagaa gtcagccctg gggcaagtct gggtaccatc actttctggc cttaggcttt |
| 59161 | ggaaattaat tagcctcttg gcctcagttt ctatctctgt aaaacagagc taataattgt |
| 59221 | ataaacttca tgaggcagcg tgagggttca gtgaggtgat cttaagggcc cgccacagca |
| 59281 | gagctgggga gacctgacac acaggcacct tattttctag ttccacttgc cttggagttg |
| 59341 | gaaaagatgg aatctgagga tccacacttc tttgtccaag gtgactgagg actggagagt |
| 59401 | cctgggcaga aggaaaatgg cagcacgatt gggactccag ggtYgcagga aRgggttctg |
| 59461 | tctgctcaga cccaggcttg tccaatgcct ccgccttcgc taggtgtaaa ctggctcctg |
| 59521 | ggcgcagtga ctaatacctc ctgctctagg cggcccccac cccctttccc tcaagggtca |
| 59581 | gctgggagga agcaggaaat agatcacatg gcctcaaggg tgaggtcctt tgccaggcag |
| 59641 | gctgaggcca gctcaggggc aaccacaccg tggttggtcc tggagggggc cccaccccaa |
| 59701 | cccccgctca gcctctgccc atgccagggc cagcccggtc actcctggct caRagttagg |
| 59761 | aatcccgccc aacttaggct actctgggtg ggccgtgtag acgtggactt ggccaggtgc |
| 59821 | gcacctccac cctgaacaaa ctctgcgggt agtacgggga ggaagcccca gacactgagg |
| 59881 | cctttcattc ccagcgaccg ggcccgctgc cgccttcctc cacgctgcct ctatcccctt |
| 59941 | ctgggctcac gcagctccaa cccacttgac caagagccct ctgcgtgcca ggctccgtac |
| 60001 | caggcacttt tgcatgattt tcctgaattc ccccagcatc tcggtgagac aggtgttatc |
| 60061 | actggcactt tacagataca gatactaggg ccagagaggt gaaacagttg cccttcggtc |
| 60121 | ccacagctgg cgacagcggg ctggaatctg gatcgccagg ctcgcagccc ggccctctcc |
| 60181 | ccaggcggct cgcccttccc ccaccgctgc ccgctggact ctgcccgccg cgccgaagcc |
| 60241 | gcccccgccc ccgccccgcg cctggcgcgg accccgaaag tggtcgcaga gaggccctgg |
| 60301 | gaagcacgaa agctggtctg aggccggctg cgcccccgac agcggagtga ccttgctcag |
| 60361 | ccgtcgccca ctcggttttc ccagtctgtc tgttggaggg aaggggagct aggccgtggg |
| 60421 | catacgaggg cggctgggtc tcaggctggg ctttgggctg tagcgcggag cggggccgag |
| 60481 | ctaggagtcc tcaggccggc aatgaatgaa ccccggccgc cccagcccgg gcagcgggga |
| 60541 | actgggacaa agggggcggg cctgccctgg agggacgctg ccaggaacac gacccgcccc |
| 60601 | tcggaaactc cctcgctccc cgcggactcc ccggcccctc gcgagcccac ccggcccttc |
| 60661 | attccgtctc ccgaacctgc tcgggttccc gcgccccacg cttgctgcag aggcaccgcc |
| 60721 | tcctacgccc gagccgcggg ccctttaagt gctgacggcc cactcMggcc ccgaccccgc |
| 60781 | ccctccacgc ccccaaggaa acatgtcact cgggttacgg ccactacggg gagggcggga |
| 60841 | ggacaaaagg cctccgcgct gtgacgaaga ctgcgcagcg cgcgcccgcg gaaggcggaa |
| 60901 | gtgggcggtg ccaccccgcg agaggcgccg ccggcgccag gtcccagtag cgggtgggtc |
| 60961 | cttgccggtg ccccgggtcc ccgcgctagc tcgtcccaac ccctggaaga gggctgcgct |
| 61021 | tccgaggggg cagggggcag agggtagggg gcccacgcct ccgccccact ctgacccgca |
| 61081 | ggttttcaga cttgccggtg tgtgccaaac catgaattca aaatcttgaa ctacScgatc |
| 61141 | ccacgggact aaggcaggag ggggtcgcag ggggcctggt gctggaagtc cagtttgcaa |
| 61201 | ataaaccaag gagctggagg aagctgtcag gccacactag cggttccagc gctcttggct |
| 61261 | ctcagccccc acctgccggg aagggtcggt ctgcactctg ccttccacga cggcaggtta |
| 61321 | gaaaatgatt gtttgtttMc aatgaaaagt agtagagcta gagaataata tgcacaaaat |
| 61381 | gtgacatcct gcgtgatgaa accagagcaa aggagcccta aatgcaggag cgcacaggtg |
| 61441 | tccgcgcagc cgggaatcag ccagcgcctg cccgcagagt ccagggaacc gacaggtgag |
| 61501 | cgctcccggg cccgggagcg ccagggagag gccgggccac cgaggcccaa agcagcaagt |
| 61561 | gtgtcctcag gggagcttgt gcaatagccc cagtcacaga ggcaaagctc agaaacaacc |
| 61621 | ccgtcgggtg ttctggccag caactgccgc aagcagcctc ctgagtaagc ccaaaaccac |
| 61681 | tgatttccag tcactatgga tttggggctc aggtggggaa acagggagga aatccaaaga |
| 61741 | ttaataaact agttcatagt ctccattttc ctctcagagt gcaacatggt aaagtatttt |
| 61801 | actcccccac gcgcatgtat acacacagct gcacaaccga atgctggaat tatgatgtgc |
| 61861 | cattcaacct ttaattacat aagctgtttt caaaaatcac attcagcgtg gagattttcc |
| 61921 | aRagatgctg gagttggcag aYggccacca cgtagctYtc cactttccac aaatggcctc |
| 61981 | tgccagcttg ggaagaacct ccatgacctg tgtcctgaga agtcagagag caagggcccc |
| 62041 | acggccatgg cagggtggag ccgtgtaRag ggtggccagg tggccagctc gcggattcca |
| 62101 | tccagagagg agactcaagg atcatgctaa gtagggaacg gggttcccac acgtcctcag |
| 62161 | gcattcaact cctgggccag ggtccgtaac aaagggagac ttcagccctc aaagcctctc |
| 62221 | actcctcagg tctgggcagg aggtccttct gccaagcagc tcctcctgcc cttcgcctgc |
| 62281 | tttctgccag gagagacctc tgtgtagagc tggctatgag gtacctgcac catttaacct |
| 62341 | attctcattt ccatgtcccc atccatgaat catctgtgtg tgcccctatt tccacatcat |
| 62401 | ccccagggct tcaggtgggc aaggggctca gagtttacag gtagcctgag ccactttgga |
| 62461 | gctagttttt ctgttcaggg cttgacagat aaagtttcca gcttccagaa gcttctggag |
| 62521 | attcacaccc tttgagaaac aagttagagg atgcggtaag tcatggtatg ccctcaacac |
| 62581 | cagggaaaac atcagcatgg Rcagacaact gtgatactga aaagtaaagg tgaagataaa |
| 62641 | caggcttttt ccttgacagc Rgggcaccag ctgcctgtga gctcctgtct gtctggcacc |
| 62701 | tgggttattc ctctgactcc ccctcatgta agatgcccaa tctctgtcat ggtttgtagt |
| 62761 | gccactacca gggcacagat cttatcagtc ttgttcagta ctccctcttc agggcccagc |
| 62821 | acagggcctg gcccacagag gtaaaaaact gatattcaag tgacaattgt gaggcagaga |
| 62881 | gKgagggata aaaacaccaa ctgaaagggc cagacagaaa ccctctgttc atacagtagc |
| 62941 | tcatatttgc tgaagtgcac tctgtgctca gctctgtgat aagcattcta cataactttt |
| 63001 | ctttcttttt ttttttggag acagagtttg ctcttgtcgc ccaggctgga gtgcaatggc |
| 63061 | acgctctcag ctcactgcaa cctctgcctc ccgggttcaa gtgattctcc tgcctcagcc |
| 63121 | tcccgagtag ctggggttac aggtgtgtgc caccacaccc agctaatttt tcttttcttt |
| 63181 | tttttgagag ggagtctcac tgtgtcgcca ggctggagta cagtggcgtg atcttggctc |
| 63241 | actgcaacct ctgactcctg ggttcaagca attctcctgc ctcagtctcc cgagtagctg |
| 63301 | ggattacagg tgcgtgccac cacacccagc taatttttgt atttttagta gagatggggt |
| 63361 | ttcaccatgt cggccaagct ggtctcgaac tcctgacctc aggtgatctg cccaccctgg |
| 63421 | cctcccaaag tgctgggatt acgggcgtga gccaccgtgc ccagcaattt ttctattgtt |
| 63481 | agtggagaca gtgtttcacc atattggcca ggctggtctc aaactcttga cctcaggtga |
| 63541 | tccacccgcc ttggcttccc aaagtgctgg gattacaggc gtgcgcctgg ccctctacgt |
| 63601 | acgttttctt acttaatcct cacaatggct ccatgagatg gatggcatta ttattcccat |
| 63661 | ttcttaggtt cagagagatg aaataatttg tctgaggtct catagcctgg gagtggcagg |
| 63721 | agtaggcttt aaacccagtt ccatctgatc tttaagcaca gatgctattc tgcttccgtc |
| 63781 | ccgccactga gggccctggt ggactctggc ggatccctgg tgagctgtct aacgctactg |
| 63841 | tcctcatttt acagagaaca aaacaaagcc aagagagaac tgactcattt caggtcatga |
| 63901 | acctgaccct tcccagggcc ccttccatgt tcctggtgtc tcctaggaMt gaactgtcaa |
| 63961 | agtcaccctg acactccagc cttcttatgc ccatgcgtgt ggcctcactc aaccctactc |
| 64021 | tcacaaacat taaaatatta gatcaaatat ccataatccc caacaccctt cccctctttt |
| 64081 | gttttgtttt gaagagatag ggtcttgctg tttcacccag gctggagtac actggcagga |
| 64141 | tcatagctca ctgcagcctc aaactcctgg gctcaagtga ccctcctgcc tcagcctccc |
| 64201 | aaagtgctga gattacaggc atgagccact gcgcctggct aaccctttcc ctctttactc |
| 64261 | ctcagcacct taacaacccc aaataccccc atccactttt gttctcagct tcaggccccc |
| 64321 | tggtcagttc ggggctgtcg ccaccagggg gtgggtgggc ttaccgtgtg aatgcccaag |
| 64381 | ccctctagca tgtagaccag gtcttctgtg gccaagtttc ctgatgcccc ctgtgcgtag |
| 64441 | ggacagcctc caagtcctgc cacagaagag tccacgacac tcactcccat ctagaaacat |
| 64501 | aaggatggtg aaacacagtt gttgctgggg acaagaggcc acagagatga gccttgcaaa |
| 64561 | cctcccaatc gtctgttgcc tgaaattcta gagtcattct ttgctttggc ttatgattct |
| 64621 | gttgacatga caatcccaca gaaagagaca gtgcttctcc ttttagtttc tgttaaactg |
| 64681 | tttcctctgc cagcagtatc tttcctttca tggtgaactc tcattcttta gNttccagtc |
| 64741 | tgaaagccac ctgctcaggg agaccttttc tgatcaccag cctaaccccc tcccactgct |
| 64801 | gagatttcag acccctgttg tttactgtga ttcatttatt accccccccc cacttacccc |
| 64861 | ccaccacctc caggctactc tcactcagtc agggccttgc cacacgaggg ccacatccca |
| 64921 | tcttcccagg tcagtcacct cccactatcc caaatgacta tttcactttc tcctctcttc |
| 64981 | ttaaacctct atcaccccct tccccatcct cattctcatc tgctgcttta tggaaatgga |
| 65041 | tgcaatcaag agatgtctcc attgattccc accatcacat ctacccacct gcctacatct |
| 65101 | gggcctacag acccactgtc cctcatgctg cctgagatgg acccYgagct cctagccaag |
| 65161 | gccaacccct ccaccttggc gtgaggccca ttcccacctg cttcaggatg tgctccagta |
| 65221 | agtctctccc tctctgctgc aacctgttac tcccctccac tgggtcatct tcacacagcc |
| 65281 | tgcgatctct cccatctttt acaaaccttt ttggaaccct ctaacctcca atttctttcc |
| 65341 | ccccattcaa agcaaagtcc ctcaaaagag ctgttcccat ttaacatcta ccattctcat |
| 65401 | ttgaacccat tcgaaacagg tttgcgtccc cacaactcct ccactgaaac agctctagtc |
| 65461 | aaggccactg gtggcctcca tgtagctaag tacaaaggtc aagtctcttc atctttgcac |
| 65521 | ttgactagcc gcatcatctt ggacaccttc tccatgtggc ttccagggct ccacatccct |
| 65581 | tggttttcct cctagtgctc tagcctctcc tgaggcctcg atccagtctt tgctggctct |
| 65641 | tcctcacctc cctgatctcg gttggagccc ttactgctca ttcctcagac agcttctcta |
| 65701 | tctccactca ctcccagtta actgcaccca ggttaacaca cctgcctata gatgtctcga |
| 65761 | gcttcacaga tcagaacacR aactgctgag atccggtcac gcacctctgc cctccaggcc |
| 65821 | tccttgccat gtctctcacc tggattgctg taggctccaa actggttctt ctctctacag |
| 65881 | tgtattctct acacagcacc cagaggagcc tctttttttt ttttgataag aagtctcact |
| 65941 | ctgttgccta ggctggagtg cactggcgcc atctcagctc actgcaaatc actgcaacct |
| 66001 | ccacctcccg ggctcaagtg attctcatgc ctcagcctcg caaatagctg ggattacagg |
| 66061 | ctcacgccac cacgcccagc taatttttat atttttagtt gagacagggt ttcaccatgt |
| 66121 | tggccaggct ggtctcaaaa ctcctgacct caagtgatcc acctgcctca gcctcccaaa |
| 66181 | gtgctgggat tacaggtgtg agccactgtg cccagccgag gagcctctca aacggaacat |
| 66241 | ttcagatcat ctccctcccY gcccctgctc aagactctct gttagcttcc cagctcagtc |
| 66301 | tgaaagagtc aaagtcctac aaggcccccg atctggcctt cctctaaccc tgacatctct |
| 66361 | gcccccctct cctcagtcac actgctcccg ccacatgcct cctcctgatc cctgaacaca |
| 66421 | gcaagcacac tcctccctca gggctctgcg cctgctttcc tgggtggggc tcccttccct |
| 66481 | ccccctccct gctagggatc cgcagggctc actccctcgc ttccctcagg tctctctcaa |
| 66541 | atgctacctt ctgagtgagg ccactctaca gaaaggcctg gcactgcctt aacctgcttc |
| 66601 | attttttctc cacagtgttt atgaacacct ggcctgctgt atgtttgttt atttattatc |
| 66661 | tgtctcatag tactagagtg tcagcctcat gagggcagag actctgtttt ggtcacagct |
| 66721 | gaatccttaa tgctgagaac agtgcctagc acactaggca ccgttaggtt aggttttaat |
| 66781 | aggtgaataa atggtcttac aatgtgctat ttacacgcct gtaatcttcc atctgtcttc |
| 66841 | cccacccacc ataacatgag ctcccagagg gcagggattg ggtcttgtta ccattgtcta |
| 66901 | agtcagcact taacctggca catcgatcca tatttccgaa gtaactgatg gaagagggag |
| 66961 | gtcagaggaa gctactgcca gacgtcacac agctcaacct gttccctgat aaattttcaa |
| 67021 | aaaggggatt ctgagatatc actgccatga aacagagaag cacagaggag caggagtgtc |
| 67081 | ctgagcRtga tcaggatgaa gttttgcctc agactcacct cagctgtgca gaattcccct |
| 67141 | ttacctgcct ggcaaggcat aggagaggaa gtcactcttc ctgtctgcct ggctgggaaa |
| 67201 | cgcatgagct gaaggaaagc ccaggaacca gggcagggta tcacagtaag ctgaaaaatg |
| 67261 | aggagaccac agaactacga accttaacct tgacagccac aactctgagg aaaccgaccc |
| 67321 | agaacataac agactcacct ggcttagaca cgaacctcta ggggataatt gagaggaaaa |
| 67381 | aaaaagtggc tggaactgcc atcactatcc tagagatgga aggttaggcc aatgctacag |
| 67441 | caaggtagct gtggtcagac actaagaatg ctccttctat ctggctgcca gccaatggat |
| 67501 | ctccattctg gaccagccca cgagaagcaa acctcaaagg aaactaatct gaggtcttag |
| 67561 | ctcaatctgt ggggaacggc attaaagcct ctccctctga gtgacctctg ctagcttctc |
| 67621 | tacctcctgc ttcctcatct gcttctgcta cacacccgca cactgaaaac cctgtatatt |
| 67681 | gtatgagtcc tccctgaacc ccacatcagt cctgaggtgc aattctgcct agtcatcttt |
| 67741 | cctcttccct caacagcagc ttactttatg ttcttcaagc ttcactgagg cctcttttgc |
| 67801 | aaatcctccc agatctcctc agctgggatg gggcccctct aggcttcctg agccccatgc |
| 67861 | ttcctccctt catggcatct gtcataatgc agtgggattg ccatgtaact cccttgactg |
| 67921 | tctccccaac acagagYtgt acacttcaca tctgggcagg gtcaccatga ctgtgtccac |
| 67981 | cattgccagc ttggaacctg gcatactggc atcagtaaat gtttgctgaa agaataaatg |
| 68041 | ataacaagct gtcctgccca ccgtgacctt tgggagaatg ggcatatgct tttgattacc |
| 68101 | tgcagggcca tcaaggtgtt ggccagggct tgaccatagg tgtcatggca gtggacagcc |
| 68161 | agggcagcca gaggcacttc ctgcatgaca gcagaYagca tgtctttcat gatccctggg |
| 68221 | gtgcccacac caatggtgtc ccccagggag atctcgtagc agcccattga gtagaacttc |
| 68281 | ttggtgacct aaggaagcaa gcaggcactt ggaggataca gaatccacca gccaggggat |
| 68341 | ccatgcactc agaagagggg gcctttgcct gggcagaaca cttctgggta tgacgcactc |
| 68401 | agttcctact ggattacatg ctcctcacca ctccaactgg gaatgatgat cctcaatcta |
| 68461 | tgatcaggag actgaggcgt aaggggcaga gccagccgct ggagccagcc cacagtcctg |
| 68521 | aagctccaac ccccgtgctt tcctccctac ctgtgcccct cagacttcag tctcacttac |
| 68581 | ggctgtcaca tcatactgtc agacccaggt gctggaatca gtgaagttga ttccttccat |
| 68641 | gtcgttttat cctcagcaat aaaatccatg aaatcatggg tctgagatgc tagttatatt |
| 68701 | ttttttcttt tttttttttt ttttttgaga ccgggtctca ctctgtcatt cggctagagt |
| 68761 | gcagtgKtgt gatctcggct tactgcaacc ttggcctctc agactcaagc aatcctccca |
| 68821 | cctcagcctc ccaagtagct tggaccatat gtataaaaat gtatatattt tacatatata |
| 68881 | tattttttga gacagagtct cactctgtcg cccaggctgg agtgcagtgg cacaatctcg |
| 68941 | gctcactgca agctctgcct cctgggttca agtgattctc ctgtctcagt ctcccaagta |
| 69001 | gctgggatca taggcaccta ccaccacgcc ccactatttt ttgtattttt agtagagacg |
| 69061 | gagtttcacc atgttggcca ggctggtctt gaactcctga cctcaggtga tccacatgct |
| 69121 | tcggcctccc gaagtgctgg gattacagtc atgagccacc atacccggcc aatcaatagt |
| 69181 | ccatatatat atatatatat atattttttt tttttttttt ttagatggag tctccctctg |
| 69241 | ttgcccaggc tggagtgcgg tggcgcaatc tcagctcact gcaagctccg cctcccgggt |
| 69301 | tcacgccatt ctcctttctc agcctcccga gtagctggga ctaccggcgc ccaccaccac |
| 69361 | ccccggctaa ttttttgtat ttttagtaga gacggggttg caccatgtta gccaggatgg |
| 69421 | tctcgatctc ctgaccttgt gatctgccca tctcagcctc ccaaagtgct gggattacag |
| 69481 | gYgtgagcca ccgtgcccgg ccaatagtta tattttttaa tagaggtaca tcactattta |
| 69541 | aatttttgga agcccatttg cacatctcta aaaccactgc aaaatgggtc catattggaa |
| 69601 | taggtccaat atgggtccaa ttggaataag ccatactgga atagggtggg cccctaatcc |
| 69661 | catacgactg gtgaacttaa aaaaagatgg cctcacccaa ggagctgctg cccacacggg |
| 69721 | gctgctggac aaggtgcaga gggatgtggc agggaaaggg gaccagaaga caccagtgat |
| 69781 | ctgctaaata aatgaaatgg tgacgtcgct tccccagtgg ccctgcagag tgtggggcgg |
| 69841 | cagtggctgg cctttagggg gcagcagggt gcaggggaag gagcaggcct ttgaagtcac |
| 69901 | gagatgctgg gttaaagtcc tagttctgct gttcactagc tgtgggcctt ggcaagcaac |
| 69961 | ttcacttttc tgagcttcaa tttctgcttc tgaaaaatgg tgatgaccct ttggtcataY |
| 70021 | ggcaaggatt gtggcaactg aggctaagac ctggagcagg gcagctgctc tcaagaaaca |
| 70081 | gttgctactg ttaccattcc acctatctgg atgccacaaa gtatcctggg aagaagtggg |
| 70141 | ctttggagag agaaaaaatg gggtttgata cctgatttca ccatttgtaa cctgtgtggc |
| 70201 | aagttattta gtgtcagttt tctcaactgt aaaagggaat aataatacca aatttgcaca |
| 70261 | gtggttttga ggactaaatg acataagttc ttgggctggt caaactcaat tcccatgcca |
| 70321 | tgttcttctc agtgttgacc tggtgactgt tcagaggaca tcagggttgt ggcctttcaa |
| 70381 | tcactacaag gaactatggg aactcctggg taaggagccc ctcctcagag aatcacctca |
| 70441 | acgaccagac tggaaactga attggaggtt ttcaggctgg atagaggcca ggggcttcaa |
| 70501 | ggactacatg ttcggctagg aggggggcct ctagggagca agtgcagggc tgtgctcaca |
| 70561 | gcaggagctt aatgaaggag ctgggtgaat gaatgaagtc aggaacctgc cagaaagggt |
| 70621 | ggggagagga acctatgcgc tcaggggcag acaaggtggc caaccctcac caaacccccc |
| 70681 | gccctgacac atgcacacac ctcagctact ttagctgggg agatcttccc ttcataaggg |
| 70741 | cagccaagag cacaggagac gtacctgtgg gaagacaggg gaggaatgag gtcagtgtcc |
| 70801 | tgagcttttt ggctagcaga actgaggcag ggctgtttaa cacacatttc tgatcagtgt |
| 70861 | gcaatcaaca cctgcagctg ggcggagtaa gggcagggat ggtacaattc agtgccagga |
| 70921 | ggaggcagta gagagcaatg gcagggaact gggggacaat ggacctacaa ttgaggaagc |
| 70981 | agaagttgca caggcctcag gagaggcagc caaggattta caataagcgg aggaggagct |
| 71041 | agagagggca aggagagtaa gcagctgggt tgtaaggtgg ggagatggag caaaactacg |
| 71101 | tgccagagga gctggggcca gtactccaaa aggagaccaa agactgggta aaaccaaact |
| 71161 | cagaacaaga gctgagttgg atatgagtca gtaacaggct gggctggcct gaggaccaag |
| 71221 | ggatgagctg ccccagtgag atgctagagg attcattcat tcatttattc acctgatgtt |
| 71281 | tactgagttg cctgctatgt gccaggcact ggcacagagc agtgggcaag actgataagg |
| 71341 | ttcctgccct ctcagagctt atctcaagtt agggagatgg ccaataaaaa agcaaccaag |
| 71401 | taagtaatgg agaaaatctc agaagtgatg tgccacagag gaaagatggg aaatgatagg |
| 71461 | aacaagtgga ggaggccaac tagtgagggt ccaggaaaga tccctctgag gacaggatgc |
| 71521 | tggagccgag aggagccggg ggtggtctgt gagaagagca ttccaggcag agggaagacc |
| 71581 | tgggcaaagc cctgagacag aagagccagg catgctccag acagaacaaa ggacagctga |
| 71641 | ggcagacaga gcaaggggaa aggaagtatg agatgaggag gtcagcaagg tgggcacagg |
| 71701 | tctgatcatg ggtcttaggg ctatggtaag gagggtggat ttgggatgta ggtgacttct |
| 71761 | attcccacct gcccttttgg gagctgtctt taaaaccctt tagagtcaag caaattctgc |
| 71821 | atattaagag gcataataaa tgttcaacag ataaataatt tcagactcaa agatctcagt |
| 71881 | gtacttttcc attttgatgc tcccacttat tctccaacca ctgctgagca agaaaaaggt |
| 71941 | aagagagatg cttagaaaga cacagatctg ggagttgggc caatgttact ggagcacctg |
| 72001 | cttccccatt catccacctg cccgtcagtg actgagtgcc cactgtggct aggccccaag |
| 72061 | ctgggagagt ggagcacccc agggactggg agagatgtgg gctttgccct catggagtgc |
| 72121 | acggtctagt ggggtggggc agggagggaa tttaacaagc agctaagtgc tacatttaga |
| 72181 | ggatgtcccg acaccatggg gtcccagaga agggcactaa ctccagctga aggactggag |
| 72241 | cagcttctca gaggaactga ctcccacaag aagacctgaa ggatgagcag gagttatccc |
| 72301 | cgagaagcat gatgtggagg aagtgttctt tattttttgg agacagagtc ttactctttc |
| 72361 | acccatgctg gtgtgcagtg gcatgatttt ggctcacttc agcttggacc tcccaggctc |
| 72421 | aggtgatctt cccaccttag cctcctgagt agctgggact acaggcacat gcaaccacac |
| 72481 | ctggcttttt ttttgtagag ctggggtttc accatgttgc ccaggcccaa gccagtcaca |
| 72541 | aactcctggg ctgaagcaac ctgcccacct tggcctccca aggaaggttg ttctgagtct |
| 72601 | agagagagca cgtgcaaagg cccagaggca agagtggagg catgtgacaa acagaaagaa |
| 72661 | gttccagttg ggctggagtg taccatgaag aggaagcagg ggccagaagg gaacttgaac |
| 72721 | ggtacggcta cagaaaagag ccgggaccaa atcatgcaRg gtttgtaaac ctcattaaaa |
| 72781 | agtctgcact tgattagtca ctgaaagctt tttaaccagg agggatgtga tcagatgtgc |
| 72841 | acttttgtgg ggtttttttg gtttttgttt ttgagacaga gtctcactct gtcacctagg |
| 72901 | ctggagtgca Stggcgtgat cttggctcac tgcaacctct gcctcctggg ttcaagtgat |
| 72961 | tctcccgcct cagcctcctg agtagctggg attacaggtg catgccacta cgcccggcta |
| 73021 | atttttgtat ttttagtaga gatggggttt ccccatgttg gccaggctgg tttcaaactc |
| 73081 | cttacctcag gtgatctgcc cgcctcggcc tcccaaagtg ctgggattac aggcatgagc |
| 73141 | caccatgttc agcccagatg tgcacttttc aacgatcatt ctggctgcaa tgtttttttt |
| 73201 | tttttttttt tttttttgag atggagtttt gctctgtcac ccaggccgga gtacagtggt |
| 73261 | gtgatctcgg ctcaccacaa cctccgcctc ccagattcaa gtgattctcc tgcctcagcc |
| 73321 | tcccaagtag Mtggaattac agctaatttt tgtattttta gtaaagacgg ggtttctatc |
| 73381 | cactaccacg cccagctaat ttttgtattt ttagtagaga tggggtttca ccatgtcggc |
| 73441 | caggctggtc ttgaactctt gacctcaggt gatccacctg ccttgtcctc ccaaagtgct |
| 73501 | gggattacag gtgtgagcca ccatgcccgg cctctggctg caatttgcac agcagtgtga |
| 73561 | ccttacagta gagtattcgc ccttcctggg cctcggtttt gtaatttata aagtgaggat |
| 73621 | aacctactcc ctgctgggct attgtgaggt ttcaattatt atagataata cacacacaat |
| 73681 | gcctggaacc actgatatta gagatgtgat gttggaaaca tagaagccta ggtaattctc |
| 73741 | cagggatgtg tgaagagcta gtgataaagc caggactcaa ctgacctcca caaacccgtt |
| 73801 | agtgtcctgt cctgctagat tcctttagca tgatagcagt ggtgctatca tggctcactg |
| 73861 | cagcctcaaa ctcctggtct caagccatcc tctgcctcag tctcccaagt tgctaggact |
| 73921 | acaggcgcat gctatcatgg ctagctggta aactgaccca aaattcaggt ttgagagaaa |
| 73981 | gtacttccta ggatttcttc tgaccacact tatttgggaa aagatgtcta ggtgtggagc |
| 74041 | tcaggctgcc cccacccagg ataagaagtt tgtggcagga ggaccacttg agtcagagtc |
| 74101 | tagccccatt ccagaacggt acagaggaaa ggataccaat gtgctctgac tcacccccgc |
| 74161 | acagaaatat tggctgactg cgctgccttc aggattgcgt caaacctctg aaaactctcc |
| 74221 | tctatggaac aattgatgtt cttcttggtR aagagctctg aggcagctcc aaagatgact |
| 74281 | acttccttgg ctccagcagc aacctgccaa catccaggtg aactcctttc agcacttttc |
| 74341 | tctttttttg tttgagatgg agtcttgctt tgttgcccag gctggagtgc agtggcatga |
| 74401 | tctcggctca ctgcaacctc cacctcctgg gttcaagcga ttctcctgcc tcagcctcct |
| 74461 | gagtagctgg gattacagga gcccaYcacc acgcccggct aatttttgta tttttagtag |
| 74521 | agacagcgtt tcaccatgtt ggtcaggctg gtttcgaact cctgaccttg tgatccaccc |
| 74581 | acctcagcct cccaaagtgc tgagattaca ggtgtcagcc atcatgcccg gccagcactt |
| 74641 | ttctctttaa agaacactga cacagagtag caaaaaattt ctcttaaggt aaattttatg |
| 74701 | ttatgtgaat gttatgggtt gaattgtgtc ctcctaaaaa tatatgttga aggattaaaa |
| 74761 | cccagcacct cagaatgtga ccttatttgg aaacagcgtc attgcagata tcattagtta |
| 74821 | agatgaagcc atactggaat agggtgggcc cctaatccca tacgactggt gaccttaaaa |
| 74881 | aaagatggcc agccgggcgc agtggctcac gcttgtaatc Wcagcacttt gggaggccga |
| 74941 | ggcgggcaga tcatgaggtc aggagatcga gaccatcctg gctaacacag tgaaacccca |
| 75001 | tctctactaa aaatacaaaa aattagccgg tcgtggtggc acacgcctgt agtcccagct |
| 75061 | actcRggagg ctgaggYRgg agaatcactt gaaccYggga ggtggagStt gcagtgagcc |
| 75121 | Ragatcatgc cactgYactc cagcctgggY gacagagcaa gactctgtct aaaaaaaaca |
| 75181 | aaaacaaaag atggccaagt gaagacacag agacacagaa aaggtggccc tgggaaaacg |
| 75241 | gaggcaaaga ctggagagat gcatctacaa gccaggcaat gccaaggact gctggccacc |
| 75301 | accagacccc agcagagagg cataggacag attctcccag aggcctcaga aggaaccaat |
| 75361 | cctgctgaca ccttgatttg ggacttctgg cctccagact gtgagagaat aaatttctgt |
| 75421 | tgtttttaag ccacccaatt tgtagtgctt tctttttttt tttctttttt ctttttttga |
| 75481 | gacaaagtct tgctctgtca cccaggctgg aatgcagtgg catgatcttg gctcactgca |
| 75541 | acctccgcct tccgggttca agtgattctc ctgcctcagc ctcctgagta gctRggatta |
| 75601 | caggtacctg ccaccaagcc tggctaattt tttgtatttt tagtagagac ggggtttcat |
| 75661 | cgtgttagcc aggatggtct cgatctcctg acctcatgat ccgcccgccc tggtctccca |
| 75721 | aagtgctagg attacaggcg tgagccaccg cgcccggcct gtagtgcttt cttacagtag |
| 75781 | cactaggaaa caaacacagt aatttctctt aattaaaaaa aaaaaaactt tgattttccc |
| 75841 | caaggaaacc accaccactg ccatcaccac cctggaagtc actcttgaga aaaagctttt |
| 75901 | ttttttttct tgctgtgtca cccaggctgg aagagtgcag cggtattatc atggctcact |
| 75961 | gcagcctcga actcctagtc tcaagccatc ctgtgcctca gtctcccgag tagctaggac |
| 76021 | tacaggtgca tgccatcaca gccagctaat tgattttttt tttttttttt gtagagacaa |
| 76081 | ggtctcctta tgttgctcag gctggtctca agcttctggc ctcaagctgt tctccggcct |
| 76141 | cagcctccca aagtgctggg attataggtg tgagccactg tgcccagcca aaaaaaggtt |
| 76201 | tttgcctaaa tacagaacca acgcctccaa agatgttctt gtctaaaatt gtaagaaagt |
| 76261 | tggcagatgc cagcaactac tgttgaaact cttgtggaga gtttggttac atttctctaa |
| 76321 | agaagtggat aatcaaaaga gctaacattt attaagcagt taccttttat ctggctcttg |
| 76381 | ctaagcaatg tacatgcatc ctatcttctg aaatgctcac aacttcaggt actatcattt |
| 76441 | agccctattt tataggcttg tcaacttgtc aagatcacag agcagtgagt ggcagggcca |
| 76501 | ggtttgcccc agggtctggc tgacagacaa gctattcaga tggacatccc aggacagggg |
| 76561 | actgaggcgc caagacaagg cagggaccaa tgccatcaat ctcatttcag gagcccccac |
| 76621 | caacaagcta tcctcttacc gctgcctcga agcctttcaa atttggggtc aggactgggt |
| 76681 | agttgatgcc aggaaacttc tgaatgccct tcaagacttc agtgtggtca cccatctagg |
| 76741 | aaccaaggga gacattgcca agtcatcacc aagagcagac agagagttca gttagagaaa |
| 76801 | accttttcat cacaaaacct ctgttctcca gaactcttag cgagtaattc cttctgcaga |
| 76861 | gctgagtctc tccaatactt ttactgtttg tttgactgct gatggctcag aagggccctt |
| 76921 | ggtttggctg agatcccaat ttctccatat ggcccatact gccagcttta catatgcaga |
| 76981 | aacagcagga atactactac cagccattta ttgagaatgt actctgggcc aggcactgag |
| 77041 | ctcagctctg tgttaagcac tcctttgtaa caggatgttt cttcttcact taactatctt |
| 77101 | tgtgaagcag gagttgtcaK tcttcctctt ttacaaacaa agaaaccaca gacaggcata |
| 77161 | gagggtttta gactttaggg ctagaagagg tcatctctaa cactgaggag gggaaggcct |
| 77221 | tagcaatgtc actgagcaag tcagagcaga gccaagactg aacccaaggc cctgaatcct |
| 77281 | gggtcatgtt cttttgctgc aacacagtgc tatgaatcac ctgagacctc tcaaagccag |
| 77341 | gggcttggaa acctatgttc tcaacttcta cttgcttcaa aggcaaatgc aaaacttttc |
| 77401 | atccctRgag aaaggccttt cattgagggc tagggctcac ctggggaacc cacttaggag |
| 77461 | acacaaagct ggtggtttct ataacagaga gtcctgcttc agaaagcatg tctatcagct |
| 77521 | tgattttcac tggagtagat acgatattct atagtggaga gagaaacacc aaggcagcaa |
| 77581 | agttagtaac aaaacagcct caaaatgcag tacctgttag ttcaagtact atagataatt |
| 77641 | ttttgcagtg gatgcagctt ctcacctgaa aaggagttat ttacatagaa acattattct |
| 77701 | gccatgcagg ctttcccgta agtaaatgtg tgttaattta taatcccatc ataaacaaac |
| 77761 | tgagagcaat gtgtgatggc ctgaaagggc agagatgact aaccctctgg ggagtatggg |
| 77821 | atggggcccc caaagtatca caccctcctc gcttctgtct cccaattggc ataggacttc |
| 77881 | agcctcatgt gttcagcgta tagcttcttt atcttcctcc ccaccatgtc cagtagctca |
| 77941 | acagattcca tggattcctc tttccaaatc taccatcatt atcctgtata taacatggca |
| 78001 | tagtctcaag ccactaattc atcttacaca ggaatcctcc taaaatgcag cttccaacct |
| 78061 | tccattctct tgcatcaaaa gctttctatg gctccctaat atttacagga ttacgcaaaa |
| 78121 | aattctaaga actccaggtc caggcggaca cattggctca cgcctgtaat cccagcactt |
| 78181 | tgggaggctg agacaggcag atcatttaag cttaggagtt tgagaccagc ctgggcaaca |
| 78241 | tggcgaaacc ccctctctac aaaaaataca aaaattagcc aggcgtggtg gcacgtgtct |
| 78301 | gtgatccagc tactcaggag gctgaggtgg atcactggag cctgggaagt cgaggctgca |
| 78361 | gtgagcaatg attgtgccac tgcactgagt gacacagagc aagaccctgt ctcaaacaaa |
| 78421 | aagaacacac aaaaatactc caggtccttc tcgctaggtc ctccccttgc ctgccttctc |
| 78481 | cagtttctct cccactgctc taacttaatc tttccacaaa aWtttttttt ttttgagatg |
| 78541 | gagtctcact gtattgccca gagtggagtg cagtggcaca atctcagctc actgcgacct |
| 78601 | ccgccttccg ggttcaagcg attctcctgc ctcagcctcc agagtagatg ggattWcaca |
| 78661 | agcatgccac caccccagct catttatttt agtagagatg gggtttcacc atgttggcca |
| 78721 | ggctggtctc aaactcctga cctcaggtga tctgcctgcc tcagcctccc aaagtgccag |
| 78781 | tattacaggc atgagccatc atgcccagcc tgtcaaataa tttttgaaag ccctccatgt |
| 78841 | tgcaggcatt gtgctaggtg ccggggatat agcagtaagc agaagcagaa cagacaggat |
| 78901 | tYtgcccttc Rggaacttac acactattgt ttccaacaag cactcactat taaagctagg |
| 78961 | atggacgtgc taaaaaaaaa aaaaaaaaaa aaaaaaaaga gaaaaagaaa aaagcttttc |
| 79021 | ttattgaaaa aataatcaat atatgattct ttcagaaaaa catgccaaaa acaaaagaaa |
| 79081 | accaccctta gaatctccca gtgataacgc tattaacacc ctggtgtgta actttctaga |
| 79141 | tctgtgcagc ccaatatgtc actagtcaca tgtggttatt taaatttaat aaaaattgta |
| 79201 | taaaaataaa aatacaattc cttggccaca ctagtcacat ctcaagttct caagggccac |
| 79261 | atgggtatag tggccaccac atggaaaagc tcaggtccag aatatttcca tcttcacaga |
| 79321 | aagtgctggt ctaggtccta gtctagataa atgctcccat acatatataa acttttttct |
| 79381 | tacataaaag tgttaagatt aaaaaataca tggccaggcg cattggctca tgcctgtaat |
| 79441 | cccagcactt tgggaggctg aggcaggtgg atcacctgag gtcgggagtt caagaccagc |
| 79501 | ccggccaacc tggtgaaacc ccatctctac taaaaataca aaaattagct gggcatggtg |
| 79561 | gcaggggcct gtaatcctag ctattcggga ggctgaggca ggagaatcac ttgaacccgg |
| 79621 | gaggaggagg ttgaggtgag ccgagattgc gccattgcac tccagcctgg gtgacagagt |
| 79681 | gagatgccat ctcccaaaaa cacacacaca cacacgcgcg cgtgcgtacg cgcacatgca |
| 79741 | ctatttgttt ttgtttggag acagaatcgt gctctgcctc agcctcctga agtgctggga |
| 79801 | ttacaggcat gagccaccac catgcccagt cacatgtact attttataaa ctgcttgagt |
| 79861 | cactttatat atacctatgt tattaatggc ttcataatat ttcattttat aaaaagtcca |
| 79921 | tagtgtatct gacccaactt tcctcatact aaatatttaa gttgtttcca actgttctgt |
| 79981 | gcttaccaaa caggtttgta aggaacaccc ccaaggctaa atctctacat ttggccttcg |
| 80041 | tgacttgctt tggataaatc cccttacatg gaattgctgg gtctacccca ccccactggg |
| 80101 | ctgtggacct ggggacacca aacctgttgc accaccattc agtttatata tatacatata |
| 80161 | ttttttaata agatagagtc ttgctatatt gcccagactg gtctcaaact cctgggctca |
| 80221 | agccatcctc ccacctcagc ctcccaagta attagggatg acaggcatga gccacccatc |
| 80281 | cagcttgccc caccatttta gatcatttaa ctgtcacagg tgtctgtccc tgtttggcct |
| 80341 | cctctactct cccctaactt gtgcagagga atcacatctt gcatagctct gtcaactgcc |
| 80401 | attgcaccta tcacacgtaa tactcaaagc agatgcttga aaaaactgtt tttttggctc |
| 80461 | atttccaact ttaccttttc attttgtagt ccatctcggg gaccaacttc cacaattttc |
| 80521 | acccgctttg gtaaagtgcc catagatgag gtgctgacct ttggtttaaa agaggaaaca |
| 80581 | aaaagtatga ggaagagatt gccacaattc ccagggagac caatatgaaa gaatggatct |
| 80641 | atcttcacta ataatgagac aagaagaaat atttctcacc atttaattca catttggtct |
| 80701 | attttagaat taagaacaaa aagaatattt cacttagtaa caggaacaag caagagattc |
| 80761 | atccttccag taccggttct cccctgaagt ccagactcct tttccactgc ctacctgaca |
| 80821 | gctctattcg catctcttca tatagcagtc ccgaaacaga actctaattt ctcctaaagc |
| 80881 | ccatctgact ccatcctttc catctctgtc aaaggcatcg catgtaccct actgcataag |
| 80941 | ccagaacttc attgagccat ttcacctatt caccatggcc tataggactc tgtatgctgc |
| 81001 | tgtatgtatg ttagaagtag tttaaaaagg ccaggtattg cggggggcag tggctcatgc |
| 81061 | ctgtaatccc agtactttgg gaggccgaag cgggcgatca cttgaggtcg ggagtttgag |
| 81121 | accagcctga ccaacatggt gaaaccccat ctctactaaa aatacacaaa aaatagccgg |
| 81181 | gtgtggtggt gcacgcctgt aactccagct actcaagagg ctgaggcagg agaattgctt |
| 81241 | gaaccaggga ggtggaagtt gcaatgagcc aagatcgtgc cactgtactc caccttgggc |
| 81301 | gacaSaggaa gactccattt ccaaaaaaaa taaaaaaagg ccaggcacgg tggctcatgc |
| 81361 | ctgtaatccc agcactttgg gaggccaagg caggcagatc acttgaggcc agaggttcga |
| 81421 | gatcagcctg gccaacatga tgaagaaacc ccgtctctac taaaaataga aaaattagcc |
| 81481 | aggtgtggtg gaatggacat gtagtcccag ctactcaaga ggctgaggtg ggagaatcgc |
| 81541 | ttgagcccag aaggttgagc ctgcagtgag ctatgactgt gccactacac tccagcctgg |
| 81601 | gcaacagagc gagaccctgt ctcaaaaaaa aaaattaaat gactctatat gtcacttgat |
| 81661 | gctttccaat taagaaaaga aaagactgta tgagttgcct cctctgacct cctggaWtca |
| 81721 | tcactcgccc ctacYgtcca ctcccaacct ccacccaaca ggttgctaag ctccctgcct |
| 81781 | tctcttccct ctgccccagg gttcctcatc tggctttttt taaaaaaact ggctgttctY |
| 81841 | acatctctct ctcatctttc agttgcagag aggcttcctc tgaccaccct ttgcaaagct |
| 81901 | gccattcctc ccacccccat tattttcgct attgcagagc tataattatt tccttcaaag |
| 81961 | cacgataaag aatgcctact caatgtctct tccagtagaa cataagctct ttgagggacc |
| 82021 | ttgtctgtct caactccact tcatccctag tgtttggcac cgtgtttggt acaaagtagc |
| 82081 | cactctttaa agttattaaa ataacacata aaccatgaag gacacagctg tcccagaaga |
| 82141 | gagcaattac tgagtgtggg gaggagtagt cagtcctgga cttttcgggg tatgggcgag |
| 82201 | atgcaggagg gctctcttct gttacagtca taggctgggt gatgacaagc atgggtctaa |
| 82261 | ggggaggtga cacccccagt agctgaagac agtttttaca aaatgccctg agtctcctct |
| 82321 | atataaagtc agctcaaaat cttagtcttc cctatcaagt aacacaatac tttttttttc |
| 82381 | ctctttcctc tacctgtctt gatactgtaa cttttcaaat caactttgtt ttacagattt |
| 82441 | aatctggata aatatactac aacgaggcaa agcggaaggc acactgtgtg atccgctaag |
| 82501 | gatctcaacc Ycctgaagtc ccccaggaat tccctgctag gaagcagctg gaacatgtac |
| 82561 | aactcacctg actggccttg ttttagccaa caagctttct tagagactaa ggcgatgagc |
| 82621 | cattgaattt catgacgtca acaggctaag ttggaaggtc cctgcaactt gaatctccct |
| 82681 | ccaacaggga tgttcagcct ctgctagtat atcaccctaa aactatctca ctggaacata |
| 82741 | agtttcatga gacagggact ttgttttatt caatgctgta tttctaaggM cctgggacat |
| 82801 | aataggggct cgataaataa cagttgagtg ttgctgaaat aatctaaaac ctgcttcagg |
| 82861 | ccaagcgtgg tggctcacgc ctgtaatccc agcactttgg gaggccgagg tgggcagatc |
| 82921 | acctgaggtc agaagttcga gaccagccta gccaacatgg tgaagcccta tctctactaa |
| 82981 | aaacacataa attagctagg catggcagca ggtgcctgca atcccagcta ctcgggaggc |
| 83041 | tgaggcagga gaattgcttg aacccaggag gaggaggttg cagtgagctg agatcgtgcc |
| 83101 | attgtactcc agcctggggt acaagagcga gacttcttca tctcaaaaaa aaaaaaaaaa |
| 83161 | aaaaaagctt ctaaaacctg cttccaagct gcttttatta gaacattata aactcaattc |
| 83221 | cacttccttc cttccccgta acagctcttc acataataat tcagttttta aacacttaat |
| 83281 | atttgccagg cacatacaag agccatttac atatttattt atWtatttat ttatttgaga |
| 83341 | tacagtctca ttctgtctcc aggctggaga gcagtggcgc gatcttggct cactgcaacc |
| 83401 | ttcacctccc aggttcaagt gattctcctg cctcagcctc ccgagtagct gggactacag |
| 83461 | gtgYgcacca ccacacctgg ctaatttttt gtattttagt agagacagga tttcaccagg |
| 83521 | ttggccaggc tggtcccgaa ctcccgacct caagtgatcc acccacctca gcctcccaaa |
| 83581 | gtgctaggat tataggcatg agccaccacg cctggtgagc catttacata ttaacccata |
| 83641 | ctgtcctctc aacaatctta tcaggtagga actatcatca tccccatttt actgtaagaa |
| 83701 | atctaaggcc ccacttggga ggctgaggca gaagaatcgc ttgaacctgg gaggcagagg |
| 83761 | ttgcagtgag ctgagattga gccactgcac tccagtctgg gtgacagatg acagagcaag |
| 83821 | acaccgtctc aggaaaaaaa aaaagaaaga aaagaaaaga aatctgagac ccaaagagat |
| 83881 | taagtaatct tcccgaggtg acatggggag ttactNNNNg aggcaatatc taaacctggc |
| 83941 | agtgtggctc tagagcacaa gctcctaacc tctgggccac tgttgcctct ttaccaccgt |
| 84001 | tcgttcggtc cacagtcttc tcctggttaa acatcccgtt cctcctctgt aaagctctgc |
| 84061 | tcagagaggg tccccctaac cacacaagta ctgtgccaca gtgtcctgag gtatggtgta |
| 84121 | cctgcatcaa caccaacagc gaatactgac taaacccagt gttaaactac aggccaggga |
| 84181 | ctattattat cccaactccg taggtgaaga aactaagcct cagaaaggtt acccaaaaat |
| 84241 | cacaattaga aagtgacaga gcttggactt gaacccatat ctaggcctaa tactaatact |
| 84301 | ggttcctatg cttggaaccc catggagggc catctcatta caccaaatat ttcttaaaag |
| 84361 | cttattaata tctactattg cgtttaactt tccaagttgg aattgccatc ttcattttac |
| 84421 | acatgagcag gtggtgcagg tgaatggcca agtaaagata ttaacgtggc tttctgagag |
| 84481 | ggacccagtg ccaggagtgt atttttccct ctatacagtt ataactcctt ctcaactccc |
| 84541 | taaggtaaaa ataccaccac cgactgatca cttattagga gccagaacac tgccagatgt |
| 84601 | gttacttaca ttttctctaa tttccaaaac aaccccaagg gaatgttgtc cgttttacag |
| 84661 | acgaaagagc tgaggtttag agaaagtaaa taactatgtc attaagtcta gaaagtggca |
| 84721 | gaactattgg gacaccctag cttcagcttt tattttttct gctccataca gctacgtctc |
| 84781 | ccacactttt tctgcgctga gaatgggaga ggggaaaaca tcaggatcga acaggatcaa |
| 84841 | agtgcaattc caagggaatc acagcttggc tccaatcaac gcaagtcccg ctcggtaggc |
| 84901 | tgggatagtt ccccaagcat ctctaaaagc aacaacagtt ggaaatggag acgaaggaaa |
| 84961 | tggaagcgtg ttcttgattt tcactaatga agagttggag aggtctgaag ttcaattaga |
| 85021 | gggagaaagg ggccagggtc atcggaaagg gccctgggcg gggaatcagg gcacctagga |
| 85081 | tggcgtccca ggtctccatg acccagacaa gtcactccac ctctccggac ctcagttttc |
| 85141 | ccatcccctt caaaggattt gaggggaaaa gggagggtcc aggactccaa cgccctcccg |
| 85201 | actctggcct gagagctgtc cggcctcgcc tctaagagag cagtcaccac gggccaagcc |
| 85261 | cccaaccctg acagtcagag tgcctgcagg gccgccgcct cggcggctcg gggcacttac |
| 85321 | agcccggagg gacgccaagc ccaccagtcg ccgcggaagc gccttcctca ttgctgccat |
| 85381 | cttggcccag aatcccccgc ggcagtccag ctgggccccg cgtgaccgat ggctcgtctg |
| 85441 | gtgggcgcgg ccccgccccc gcccctccgc Mgtgacttcc gaagctggct ccacccccag |
| 85501 | cctctctccg ttttgattcc gagcgcctct tccatgccag gccaagtgag acgctaggaa |
| 85561 | tataaagatg aacagaatca gcctggacac catgtaatcc tagcactttg ggagaccgag |
| 85621 | gcgggtagat cacctgaggt caggaattcg agaccagcct ggctaacatg gcgaaacccc |
| 85681 | cgtctctMca aaaaatccaa aaaaaaaaaa aaaaaaaaaa aaaaaaaaaa aaagccgggc |
| 85741 | gtggtggtgg gcgcctgtaa tcccagctac ttgggaagct gaggcaggag aattgtttga |
| 85801 | acccgggagg cgaggttgca gcgagcggag atcgtgccac tgcactccag cctgggcgac |
| 85861 | acagtgagag cctgtccaaa acaaaaaacc caaaaccaaa caaacaaaaa catgaaaaga |
| 85921 | aattaaccaa attctgtggc ttcttgtgtt tagagtgtgg gaggtgtgag gagacacata |
| 85981 | ttaatagtta cacaaacagt tgtgtcattt ctaacttgca ctataagaag caacgatttg |
| 86041 | ctactcactg ttccgagttc ttagcacgga ttaactcacg catcacgatc ttcaggattc |
| 86101 | aaacacaggc aggcagctcc agagaggggg catccttaga tttcatacct taaaaaaatg |
| 86161 | attaagattt atctaggtgt actttctgaa gggctNaaaa aaaaaaaaat ctatccaggt |
| 86221 | gaagaggata caagaaaaac tttccagacg gtgtagcatt cgcaatggcc ctgaggcaaa |
| 86281 | gaaaatgctg agaacactgt ttttctctct tttgttttgt tgKtttgttt tgtttgtttg |
| 86341 | agatggagtc tcactctgtc acccaggttg gagtgcagtg gcgcgatttc ggctcactgc |
| 86401 | aacctccgcc tcccgagttc aagagagtct cgtgcctcag tctcccgagt agctgggatt |
| 86461 | acaggtgtgc accaccacgc ctggctaatt ttagtgtttt tagtagaaac gaggtttcac |
| 86521 | cgtgttggcc aggctggtct caaattcctg acctcaggtg atccgcccgc cttggcctcc |
| 86581 | caaaatgctg ggattacagg cgagagccac cgcccccggc cagagcactg ttttcaaact |
| 86641 | gtatgttgta ccccactaat tggtcacagt atcaatttag tgggtacaaa ctttatttaa |
| 86701 | aacaaRgaac tagaatttta tctatctatc atctatctat ctatctatct atctatctat |
| 86761 | ctatctatcc atctatattt atttattaag acgaagtctc actgtgttgc ccaggctgga |
| 86821 | gtgcggtggc gtgatctcgg ctcactgcaa cctctgaccc cggggttcaa gcgattctcc |
| 86881 | tgcctcagcc tccggagtag ctgggattac aagtgcccat caccgtgccc agataatgtt |
| 86941 | tgtattttta |

Following is a GALE region genomic nucleotide sequence that includes the GALE SNP “HT014-AA” (SEQ ID NO: 4958).

GATATCCTCCTTCCAGCCAAAGCGAAAAGGTAATTTTCTATATCTTCT
TTTTCTTAATGGGAAAAAGATTTAAAAATTCATTCAAGGCAGGGTACA
GTGGCTCACACCTATAATTCCAGCACTTTGGGAGGCCGAGGTAAGAG
GATTGCTTGCGGCCAAGGGTTCTAAACCAACCAGGCGTGGTGGCTCA
CTCCTGTAATCCCAGCACTTTGG[G/A]AGGCCGAGGCGGGCAGAT
CACCTTAGGTAAGAAGTTCGAGATTAGCCTGGCCAACATGGTGAAAC
CCTGTCTCTACTAAAAATACAAAAATTAGCCAGGCATGGTGGTGGAC
GGCATAGGGAAGAGGAGGAATATGGTATCCAATCTAAGGGAAAGAGA
AGATGGGGACAAAGACAGAACCTAGCTTCCAGGAGG

Following is a GALE region genomic nucleotide sequence that includes the GALE SNP “HT014-AB” (SEQ ID NO: 4959).

TGTTACACAGATTTTTGTGATATTCTGTCACAGCCACCAAAAGCAACTA
GCATAGTGCCTCACACTTCGCATTTTTAAAATTTAGGGAGTTATTTTTTT
CCTAGAATTGTGGTATTTTAACATACTACTAAGATGAAGACCAAAAAAT
TTTTGGGAAACTGGTCACTCCTAGGTAATTAAATAATCAGAAAAAGATAT
GAGGCTGGGCATGGTGACACACGCCTGTAATCCCAGCACTCTGGGAG
GCCAAGGTGGGCGGATTATCTGAGGTCAGGAGTTCGAGACCAGCCTGG
ACAACATGGCAAAACCCCATCTCTACTGAAAATATAAAATAGCTGGGC
GTGGTGGTAAGCACCTGTAGTCCTGGCTACTTGAGAGGCTGAGGCGTG
AGAATCGCTTGAACCCAGGAGGCAAGGGTTGCAGTGAACCAAAATTGC
ACCACTGCACTGCAGTCTACACGACAGAGCAAGACTCTGTCTCAAAAA
AAAAAAAAAAA[A/T]ATGGAAAAGTGCTTGTACACAAATGATGTCCTG
GTTCTGGTTTCAGGTATTAATTGAAGAAACAGATCAATTATGGAAAGTT
CATTGTCACCGAGACTTTAAGGAAGAAAGACCCGAAGAGTATGAGTCGTG
GCGAGAGATGTACCTGCGGCTTCAGGACGCCCGAGAGCAGCGGCTA
CGAGTACTAACAAAGAATATCC

Following is a GALE region genomic nucleotide sequence that includes the GALE SNP “HT014-AC” (SEQ ID NO: 4960).

GTCCAATCCCTTCACTTCACAGGGGGTAAACTGAGTCTCAGAAGATCAAG
TCACTTGCCCAAGGCCACGCAGCTAGTAAGTTGCTGTGGTTGGATTTGAC
CGTAATAACCATCGGCTACTGAATGCCCATCATGTGTCAGCCATTTGCTG
AGTTTTTTCTTTTTTTTTCTTTTTGAGACAGAGTCTTGCTCTGTCGCCAG
GCTGGAGTGCAGTGGCGCGATGTCGGCTCACTGCAACCTCTGCCTCCCGG
GTTCAAGCGATTCTCCTGCTTCAGCCTCCCGAGTAGCTGGGACTACAGG
CTCGCGCCACCACGCCTAGCTAATTTTTGTATTTTTAGTAGAGACGGGG
TTTCATCATGTTGGCTAGGATGGTCTCGATCTTTTGACCTCGTGATCCG
CCCGCCTCGGCCTCCCAAAGTGCTGGGATTACAGGCGTGAGCCACC
G[C/G]GCCCGGCCTGCTGAGTTCTTTAATTGCATTATTTCATTTAAT
ACTCACTGTGATCCTATGAAACAGATACTATTCTTTCCACCTTACAGAC
GGGGAAACGGAGTCTCAGAATAGGGGAAATGACTTAAGGTGTCACAAT
GGCCAGTAGCAGACCCAGGAGTAGTCTTTAGGTCTGGCCGTGCACAAA
GCCCCATGCTCTCAAGCTGTAACCCCGCTATCAAGAAAGTGGTTATGC
AAGTGGGCCTGTGAGCTCTCAACGTCTAAGCCGTGCGCATGTGAGACA
GCGATAAGCTCGCGTTCTCCAGTCTCCAGGGCCTTATCATTCCTCACGC
TTTCCTTC

Following is a GALE region genomic nucleotide sequence that includes the GALE SNP “HT014-AD” (SEQ ID NO: 4961).

AAGAGTTAGAGACCTCGGCCTCCTGGTCAGTGGAGCCCTTGGCCTCATG
CCTGGTGGGCTAAGCGGGCCCAGCTGGGGTTTGAGGTAGGGGAGGCCT
TGG[C/T]TTGGCCCCAGCAGCTCCAGGGCCCTGAGTTCCTGCCAG
AGGCTGGAGAGCAGGCAGCTGCTGCTTTTCCTGGTCCTTGGTAGGGGA
GGGTCCTCAGGCTT

Following is a DPF3 genomic nucleotide sequence (SEQ ID NO: 6).

| >14:71066151-71351450 | |
|---|---|
| 1 | tccaatctgt aggttctcct ggagattgtc acaatctgcc agctctctgg gaatcacaga |
| 61 | accatcatgt ccccttagga tggcagaaga tgtggcacag cctcaatctc caacactgaa |
| 121 | acactgagag agcttgatac gttccttagg cagggcagaa acatcacagc acttcacgtt |
| 181 | aggaaccacg aaagagtggg aacctgRcac acgtgggaag ggcacatccc cagaggacag |
| 241 | aggacaaggg tggcacacac acctacagag accgcaacca catcacagtg aaggaggagg |
| 301 | acagctaacg gggtgggagg tgggagcttt ttgtttgtct gttgctgtcg ttgcttttta |
| 361 | gagaagccag ggtgggccaa atggagatat tgtctcaaga ccattacaca cagcttccca |
| 421 | aatctgtagc ccttttctgt aacctcatga aagctgtgtc agactcagtc ctggtctgaa |
| 481 | gtaccMaagc agcccaaggc aaaacctcat ctgcaagagg caatgcggcg ttgggtccca |
| 541 | aggatcagtg gggagggtgt tcaaaagagc aaaaagggaa accctctttc ataggcattt |
| 601 | ctctcactgg agacccactc tcttggtcat tcctttatga ctgagagtct cagaaggagt |
| 661 | tgtgagtgtg aggtaaaaga gacaagcaag cattttggaa tgatggggat aaaatgttgc |
| 721 | agtggcttcc ctctctggtg cccatacctg gaagaagcaa tcccaggttt gcttcgtatc |
| 781 | tgcaggtcta gagacatcaa tggaggcagc aattggagat ctcagtgtgt ttccagggtc |
| 841 | tctgactcaa ctagagggca tccaaggcct ggtggagcct gtgcaggcct gagcttcctc |
| 901 | gtggagcagc aggtaggcag ctcattccca gggacctggt catccttcct accataggag |
| 961 | ccacctgcca aagcccaccc agaggaatcc cagcgttcac aaatggaggc gccctcacca |
| 1021 | aagccactgg accagcctgt ccctgagaag agacacagag gggcttcagc aatgagaaga |
| 1081 | agaaaaccaa tttcttttgg cttcttagca gagatgctcc atgtcttggc tgtcagagaa |
| 1141 | attgagctta caaattgccc tcaccctgaa cccacttggc aacggggaga tgacctggtt |
| 1201 | gaggtctagt tctggctttt gtgaatcgac cgtgtgacgt agacagcaca gcatcaggcc |
| 1261 | aggaaggcgc ctgatgagac cgtgttctca gcctgagagc gcgctcaagg gcaggtggga |
| 1321 | gatggagcct tctcagagct cagccactgg ctcttgggct cccaggttca tccctcaagg |
| 1381 | agctggggac ctgagggctc agggcccacc actggctgcc tgggggaagt gctgccagtc |
| 1441 | tggtgtggaa agggtccaag tcagaggcct ttgcctcacc ccttccacca gcagggccat |
| 1501 | tcagagaagt cttggctcaa aagaacgttg cccacaggct ttcaagtagg tgagtggacc |
| 1561 | aaacccctgc atgcttttca ggctgtcacg cactttaaaa ccagactcac agccaagcct |
| 1621 | tgtgtccctg actacctcca cagagatcac cagaattctc ctcactggga gtcatgtgcc |
| 1681 | atccagggcc ccagaggagc cttcccttgg gccacccccc acagagaagc tatagctctg |
| 1741 | agccRacatc cacacatgga cagatacaca ccttccccca agcccaggca ttttgcttga |
| 1801 | aagcaaggtt ggtgtggctt cttcagggaa taactcaaag ctgaaatctg gctgtgacac |
| 1861 | gtgtggagaa aaggaaagat gaaacaaacg gtttattcct aatgctgtca acacaaacaa |
| 1921 | cYgagagtct aagccagcac gaagctagga tctgacaagc acctggccaa aggtgagcat |
| 1981 | ggaaggtttg ctgaaaacct ctctgatgct gggtttcatc cctgcacaca ccagcagtgc |
| 2041 | cagatacagg cgtggggcag ctgaccttgc tggggtctgg tggagtgtgc ccaatctgta |
| 2101 | cttgaagccc ccaccccccc agtcccgctc tctgctttct ctagcaggga aaagaccact |
| 2161 | gtgggggtca ttttctgcct ctgatcaggM cctgccagtt gcacctccct gccttccacc |
| 2221 | ccagaaaccc acaagcagct ctggggacac tggctgccca acacactcca ggccaggtga |
| 2281 | cttcatcagt tcaccagttt taatgctgag gatgctgtca tccaagggca tccagtgctt |
| 2341 | ggaaaagcag attaaagcaa ctaggctgag gtcatctgtt ttcttccccc gacccctcag |
| 2401 | cctccacagg gtagaaatca ctggatgcct ccacctgctt gtcttctact gaggaacacg |
| 2461 | catcctcaac agagaaacct acatatactt acaccttccg tgatggcgcg atacttgacc |
| 2521 | tacaatcacg attgcttggc agagcttaat ttcatggctg aaaagcaact tgaaggctag |
| 2581 | gccagcatgY ggtttatttt ccagccccta caggacctag agcattgcct cgcacaYagt |
| 2641 | cgatgccctg ccaagggttt taatttagcc agtaacagcc tgccctgcaa tgattcatgg |
| 2701 | caggcaaaag atgggttcat tttccttcag aggaaaaacc cccagggtgg acaaccatgg |
| 2761 | cagatgagtg gggggcagct tggatggact ctgttccctt tccYgRccag aggagcacaa |
| 2821 | atgcccccta agcatctgac cattcattgc aaaagcatga cccatctgct ttcccaaatg |
| 2881 | ccatgtggac cagaYgcatt ggtgagccag ggacgccctg cctacctacc ccgcctctct |
| 2941 | ccagccacct gctccccacc tcctctgctc tgagataatc attctatatt ttcatgctct |
| 3001 | tgctcacata tcaggcagtg tttctgtcct tttttttttt ctagtaccta atttaggctt |
| 3061 | cttcaactac atgttgaaaa tgtgcacggg gtatattttc tttttcctWt acgccactgt |
| 3121 | tgcttcccac ttcccacctc caccccactc cttagcatct atcacggcag agggaaggga |
| 3181 | ggaaNcagtg cagggaaggg agagggcagg aggagaatca gggtctcagt ggggagtaga |
| 3241 | catggaaggg caaaccaagt ccgtataaac cacaaacccc attctagcca taaaagcttt |
| 3301 | gcatcccttt gatagcagaa ttgagacttt tgaagtaccc aactattgcc aaatRtcttt |
| 3361 | catgaaaaga agcataggtg aacgaaggga agagaaggag agaggtcgcg gagccagtag |
| 3421 | ctttccaagc acaaggctcc ctttgcaacc ctggtggctg tgcagaggct gagagctgga |
| 3481 | ggagcaggac tggagaagac ttcaggaaag atgcaccttg cctggcagcc cctggctctc |
| 3541 | cctcttgtcc tggagcaaca tcaaagtttc cctttggttc attaagtagg gcgtgtgtgt |
| 3601 | gtgtgtgtgt gtgtgtgtgt gtgtgtgtgt atgtgcgtgc gYgcacgcac gcgcatgcac |
| 3661 | atgggcattc ctgttctcat catcaatatt cataacattg aaatgaagaa cagagatgca |
| 3721 | gagaggcgag caaatgagca aatgggatgc agtgagtgaa aatataacct gggagtctgt |
| 3781 | agggttgtgt tgctgaggga atatRtaggc cccaggggct cacaggcctc tgccatttcc |
| 3841 | tacaaggccc acttacctgc cctcccgtcc ccaccatggc cgcgtttatt tgctagacag |
| 3901 | ggttggtgcc agcctacagg gccattttgc tcaatgcctg ggctagaggt catgataagt |
| 3961 | aattatatct ctgtccaaga gggttctggc tgcctgccag tcctggttcc tgccttcctc |
| 4021 | tggcctgccc tgggaaatgt cgccatcagc aaaatactcc cttctcctgg tggagccaaa |
| 4081 | gaaggggaac tgtggaaggt ccctctctca aggccacctg ccctatgacc tgaggttggc |
| 4141 | actggagcag agaaagggaa ggagtgggga cagaagctgg aacaaagggc cctggggagt |
| 4201 | cacctcagga ccccctcaca gccagggctc actcaRaacc cagagagagc ctgttcccag |
| 4261 | gaacccactc acttccaaat ctttcccctc atgcagggag ttccctgttc cttctagaaa |
| 4321 | acccatggta Sggaactggc aagcccggac ccctcctcct cagagtttcc tgacagctct |
| 4381 | tcctttgggc ctctcttcta gctgcctggg ctgcgcacat ggagggggcg cccgccgcac |
| 4441 | agccaggcct ccccggggac agctgctgag aggctctaag gcagttccca ctcgctctgc |
| 4501 | ctgggaggYt gtctatcctc ccacatcccg ggtgcccagc aggaagcacc cccatcttcc |
| 4561 | tctcccctcc ttccccctgc cccactctca gctgtccact cacagaccag agggtctcca |
| 4621 | gcctccctag gaaggggttc ccagctctcc aagacccacc accagctgcc caaaggcctt |
| 4681 | gaaccaaccc atctgtccca ttgtttccac actcagggct ggtggccgtg gcctctggca |
| 4741 | gagaatcata agcagactcc caaaccctgg gaatgctcag aaatggggga gggagcccca |
| 4801 | acccccagac agctatggag cccaccttct gaccacggac cagttagact ctatggcaag |
| 4861 | ctggggttca ggtagggaga ttcataggcc tgaggcaaat taaaacagct gttccccacc |
| 4921 | atcaccatcc ttccccttca cctggtatcc aaaagcatRc tttggggatg ggagtaagat |
| 4981 | ttcaaaacgg gcaacacaca gcaggaagRg gaggagctct gtccactttg cagaggggag |
| 5041 | aggagcagga tggccagtcc ccctcttccc ttcccagcta ctggtgtcct gggcttgggg |
| 5101 | gacaggggcc tgggggcctg acccctctgg tagcagtgcc tggaaatgct tcactcccca |
| 5161 | aggctgcagg catgatccag ccctccctca ctgccttctc tccccagctg ggtgaggggc |
| 5221 | ttactttgcc ctcagaagca tccctgaaac cccacacaac caacgggcgg cctcaagaaa |
| 5281 | gagggagctg aatcagtgtt cagaactggg cagaggtttg gcctttttac aaaataataa |
| 5341 | taataataat ctgttgccca ggatcacaag cctcagaaaa aaaaaaaaga gttacaatca |
| 5401 | ctgttcactc ccacctgctg ccctccagct ccttagctcg aggatttctc cctgaggaca |
| 5461 | tccagctctc tgcctttttc caccagagat cctcacccct cccaaatgcc ctcccaccct |
| 5521 | cccttcccca ggcacccctg tctttctccc tccccaggaa tggaagcctg ctaaaaggga |
| 5581 | caccaagtca ccactgctac caggagccag gcctgggact tgcggcatcc tgtgggagtg |
| 5641 | tgagcgtggc tctggccaga gccaacttct ccctcRggct gagacatggg gaccccctga |
| 5701 | agaggggcta ggaggggcag ccggggagtg agagaagaag gggctgcttc tgtcccagca |
| 5761 | cttccacgac aggctaaaaa ccagcctggg gggcagggat gtggtcctca gggcccgccc |
| 5821 | tgggccacgg gcggcgcaac accccctacc tcctcacaca cagacccaaa gaggagacta |
| 5881 | taagcccctc ttctttgagt cctgaggcct gaggttgcca gcaacctttc ttcagcggca |
| 5941 | taaaagagca caggtctcct ccacttgccc cgaaaggaaa agtaataaca atcatctcat |
| 6001 | ttttcttcct taaaacatca aaacttttta agcggagggt tgaaaggaag cgggctgtat |
| 6061 | tcaaagcacg aatacaaaaa tgcaccagga gtgtgcaacc ctccagggta tcatgaaacc |
| 6121 | atcactgtga aacccaaacc ctcccccaac ttcaaaacct gttctcttcc ccagttctgg |
| 6181 | aggaatctgg cctccttccc tcccttccac ccacccacag gctcttccct ggttccagtc |
| 6241 | ggctgggctc agcctgccca ggcttccggc ttcctacctc gccctggggc agggacagga |
| 6301 | gcaggggagc cttgggacct gctggctcct gcttcagggg cgatgagtca gtgaggggtt |
| 6361 | ccaaaggaaa gccagaatgt caatctccta gcaggagccc tgtttctatt tccacataat |
| 6421 | gttgcctgca ctggatttgc tacagatcca aaaagcaggg tttcaaaaat gcctacagga |
| 6481 | aacaggattt ccacttccgg ttgggtStgg cagcctcacc tctctggttt gctggagcca |
| 6541 | cagtacagag gggtgaagcc cagcaaagtt ctcctggctg ttcccagcat ttgagccgga |
| 6601 | agacaaccac atcattatct ccatttggtg gcctctgcag aaaatgacaa tagctaccat |
| 6661 | ggagtgggga ccatcatcat gccagacccg ggccRggatc tttaccaatg tcacctYgaa |
| 6721 | tacctgcaaa gaccatgctg gataggccat gtttccccac cttacagaca tgtgaaatga |
| 6781 | ggctcagaga gttgaagtca ttggcccaag gtcagtcagc tagaaagtgg cagtgttggt |
| 6841 | cggaaagtga gcgttaccca tgaactgaaa caaatcatag ccctctctaa aagtcacctt |
| 6901 | tccattctca actgcattaa tcttatagaa ggagtatcct gagtaccttc attttaaagt |
| 6961 | ccccaacccc atgtacatcc ctcacctcca ccccacagcc catgcaagtc atgatttcct |
| 7021 | tggtcgcagc ccagccctgg caatataaac tccctttggt cacaagggag atttgaagaa |
| 7081 | gacgtctaac attggctaat gcacacaaga aggcggacat ggaaaacatc Yggagttctt |
| 7141 | cccagacaag catggctctg cagtgcttgt ttatgtaaca tgatgcttaa gagtcccagt |
| 7201 | gtcactctgg aataggcagg gcaagtgtta ctatctccac tttccaggtg ggcaattgag |
| 7261 | gttcagggag gttaggtgac ttgcccagtg tcacacagct actccatggc agaggcaaca |
| 7321 | caaaaactct gatccttcac atgcatccta tgattctatc tcattctctg gggttcaaaa |
| 7381 | aaaagagacc aatttaaagt ttaccaaccc cgtcctcttt tgaacacaga ggtgataaat |
| 7441 | gtgaccacgg agacttttgt ctgcttgtag taccctgaca gtgccagccc acaagctggc |
| 7501 | ttgctatccc cgctttccag atggggaaac tgaggcctcc aaagatgaag aatgggttgg |
| 7561 | gcccattctt cagggagtaa tgaagctgag aggggaactc cggggccatc ctaacgtcac |
| 7621 | tccagagccc tgaccctcac cagtgcaggc aagttccagt gccctgcctt ggcctgggag |
| 7681 | acccagctga ggctcagctt gtgtaggcag agcagacatg gggctgagat cgaacagggc |
| 7741 | tgccctatca cctgcacttc tgtggaaaac aaatttgcac atactctgaa gaggacgtgt |
| 7801 | gaagttgtag agccacagtt tggggtttcg gacttgcttt ttaaactttt aagctattgt |
| 7861 | ggcacttggg aaYatttggg gaactttttt ttctaaattt aaattaaaat gacgctcaaa |
| 7921 | aNcaattatt agcctttgga gacagcctgc tttgggtttt gtgggaagag agagagctgg |
| 7981 | agtagtcagg agaaaaaatg tgcccctctg atggaatgtc accctcccta tccctccctg |
| 8041 | ggccttcagg attcagacaa gctcaccgac cagcaggcgg aaggaaatta cttatgagga |
| 8101 | agatgaaaat tccatccggt cgggggccct ttaaatgaga ccattatgta tgtgtgtgat |
| 8161 | tccctctgag gccacaggcc cctgcaggag agtgaccccc atcgcttgcc acaggtcact |
| 8221 | ctgctgaagc gtgggggaga cccacactgc ttggtttcat gccaggctgc cctgggtcca |
| 8281 | ctggccaaga gcaattagcc tagagaggag cactcctgcc gagggacagc acaaatactg |
| 8341 | cggccgggca gaggtggccc aactagcccg agtctccggg agcgtcctca ggagctgaag |
| 8401 | aactaaaaat aacctgggcc tttgttgtga gcagccagac cacagagatg gagctgattt |
| 8461 | cagagaagcc aagcatgcgc gagggttccg ctcaccgaga cctgcccttt gggcaaatga |
| 8521 | tttttccaga aatatgtcag ttattgtgct gctgctgcga ttcaaagtca gcccaatccg |
| 8581 | ctccagccct cggggctatc ttccaacaca cagagttctc ggaagctcag cctccccacc |
| 8641 | tctttcttct aRacacattt ttttttgaaa acaagagtcc tgcatgtttg gggagcttag |
| 8701 | gagacaagag cagcccccgc actgagagtt ctaaaatgtg gccaccctgg tcgtgaataa |
| 8761 | tgaccatgtc accctaatta tttgtttaga taacaatctt ctctccccca tcctaaaatt |
| 8821 | cccagtcagg tctcagtaga caatagcgaa caaacactac gtggaaggaa gaaattactt |
| 8881 | ggaaaaagga attcagagcg tcgcatcaat actgaaggtt tctcaaagct gcagccccct |
| 8941 | gtatccagca ttcggacaca tgattgggca gccttcgctc cccttcctgg ttcttcagtc |
| 9001 | ccaccaggag aagctgaacc aggtctccag aaccaggtgg gctgagaggc agaggcttga |
| 9061 | ccgtcttcct ctgtccatag gccatgactg gtctcaccta aaggatgccc cagctctgaa |
| 9121 | ggtcaaggaa ccagcctggc ctcttgcaaa gtctctcccg atggataaca agtactgact |
| 9181 | ggctatggcg gggccttggc accatttcca caatgtttcc tcccatgtct ctgcgcgtct |
| 9241 | ccctccctcc ccgcccccat ctagtaactt taagcagtgg ctcaagacca ggctggctgt |
| 9301 | ggatgccact gctggggttc tagaaggcaa ctctttgtct ccacagacac caaggaacaa |
| 9361 | gaaagggttt caagacccca agtctagaag tttccagtga ggcttgccat aaacgacaac |
| 9421 | ttctccacag ccctttgttc ctcaggtgag aatttcagga tgcagaccct aagctggggg |
| 9481 | cacgggggct gtgccaagat ttcttggaac acaatagata aaaaaacacc acactacaca |
| 9541 | caagtaatgg atgagtggtc aggaaatggc ttcccttaaa atgggaacca atgagagggc |
| 9601 | ctcggtgcta gggtaaaagt gagaggggtt ccacagggct ggggccggag gtgttctccc |
| 9661 | aaagtgcaac ttccttccgg tagagacaag gaggtgctgg ctgaagttcc gtacatatcg |
| 9721 | ggggaggtca ggagatgcct caccctcttc gttccatgtg tccctgcccc tctgggacta |
| 9781 | tttcttttcc ttgcagttgg tctggctgga tatgtgRgag gagggcgcgt tctggtttga |
| 9841 | actgggctct gctttaggat ctccagcagc gagtcacatt ctgtgacctg gggccctagg |
| 9901 | cctggcagcc aaaggctgag gctttctctt tgagcagttc ccagcataag tggcagctcc |
| 9961 | agcttcctgc aagaaatgag acggctttag tagcagcccc tctgctaact ctggagcccc |
| 10021 | accccactgg ccctaacttc ttgtaaatcc tgtttccttt gaactttggc aatgcagaaa |
| 10081 | ccaagtccca ggcctgaaaa cgtgccagag tctgtggctc agcagaaagc aggtgctcag |
| 10141 | aggagatggg ctcaactctt atcaagaccc tcactactag cgtcatgcct ccaaactagt |
| 10201 | gtcgccgggc ctgcgccagt tctcctggaa gctcacacct cccggtcatc tccactgcat |
| 10261 | gtctgggaaa aNcttccttg gccctcaagt tgaaatttca gctgcttgga ctgtgcctca |
| 10321 | gtttcctcat ctgtaaaatg gcagtcgtcg tacctacctt gtggggttgt gatgattaaa |
| 10381 | tgagacaaga catgaacatg taaacccatt agcataaggc ctgtaccata gtgagagctc |
| 10441 | agtaagtgct agtagtagta gtatctgttg ctgttcttat tgttgacttc atcatcatta |
| 10501 | ccttctgggg gctcagccac cgggggattt aaacagtaca tgtgatagcc tcggtcacag |
| 10561 | tcatcgcaga agagtagctg gtcctggtgg aacacagagt aagcacagag gaggagagat |
| 10621 | tccattattc ggggccaatg tctggccccc gcttacccat cagccttatt tccacgtcgg |
| 10681 | tctcactgga tcccctttcc aggccccact tggagtcctc actgtcccct gaacacgcct |
| 10741 | gtgcttttcc cacaactgga gtttccccct ctacctccca gcctatataa atcctacccc |
| 10801 | cattttaggg ttctgctcaa ctYgtacctc ctccaagacg ccttctgtga ctaacacctc |
| 10861 | acatccaaat ttaaagggat cccttattct tctgaacttc caaagccaat agctctgctt |
| 10921 | ccaaagggca gaaaatgtct ctattctcca tgactttata tatttgaaga tcttaattga |
| 10981 | attcttctgg actccactct ctccatctgt gaaatggcca acagctgtac tgggatgaac |
| 11041 | ctcagaacct cttcagggtt tcattagaat gtagaaaaat gcaagtagcg ccatccctcg |
| 11101 | gcttccagaa gcttcctcac tgtctggagg ctggagcccg ggtgagagca gggcacccca |
| 11161 | gggttgtggt taacttcaaa ggacacaggc catatctttg cacctatggc tacttctgcc |
| 11221 | tggaggctct cccaggccct gaccctttct ctgcatgtcc attccctgcc tgcccttcaa |
| 11281 | gtacgtcttc ttcaagcact tcttttccac aaagtctttg ctaatctacc tcaactggaa |
| 11341 | gttgtctctt ccctcgtgca gccctgtggt actttccctt tctgtgacat ttaaatgcat |
| 11401 | tttcctttac gttacatgcc tggcatgtgg tagacatcta atcacgtttg ttttcttggc |
| 11461 | tgggYgctgt ggcgcatgcc tgtaatccca gcacttaggg aggccaaggc gcgtggttca |
| 11521 | cttgaggtct ggagttcgag accagcttgg ccaacatggt gaaaccccat ctctactaac |
| 11581 | aatacaaaaa ttagccaggc atggtggcgt gcacctgtaa tcccagctac tcaggaggYt |
| 11641 | gaggcaggag aatctcttga acccgggagg tggaggttgc agtgagccaa gatcgcacca |
| 11701 | ctgcactcca gcctgggcaa ctgagtgaga ctctgtctca aaaaaaaaaa aaaaaagaca |
| 11761 | tctacatgtt tgttttcttg aatagttcat ttggaacatg cctctttttt ccacccttta |
| 11821 | tcccctttat ggactataaa gcccttgggg gcaaaatctt gttttcctca tcactgcatc |
| 11881 | cactgtagag acttccttgc atacaggagc tgctcggaaa ctgtttgtga tctggcccca |
| 11941 | ccccctagtt tcagctctgt tctatttggt gattgttgaa actgtttgga tggcttatga |
| 12001 | gcctttttca aaataaagtt ttatatcaaa tctcaccttg gcccccacca gtgcagaaat |
| 12061 | cctggataaa attagatata gctgttggaa tccccaggga gcaagcaatg agggcttttt |
| 12121 | ttgtcatgct aaaacccgtc agtggggcaa caggaaagct tttgaagagt tatcaaYagg |
| 12181 | ggagtgatac gatcagattg cagtttagaa agatcttact agctgctatg taagagtaga |
| 12241 | tacccagtgg gggagaggtt cattcaagag gcccggggac cagttaggaa gctgctatag |
| 12301 | ctatccaggt gcgtgatgat ggtggcctga attacaattg cggcatggag aaagaaatgg |
| 12361 | gtcttgctcc catttaagca tcattgatta gatggggaga gtgataggga agaagccaag |
| 12421 | gatgccgtct gggttgtggc ttggtcagca agccagatga tacagccacc caatgggaca |
| 12481 | tgggaggaag agcagactca tgggagggag agggcaacct cagctataga catgttctac |
| 12541 | agatagggat gacctggagg ctgttgaaga gagcaggaga gaagtctggg cctgagatat |
| 12601 | ggaYttggga ttcattgtaa ttagatggca agtgaaggtc ctaggagagg aagcccagaa |
| 12661 | tgtgtgagag caaatgtctt tgaccttgtc ttcatggaga agtgtccatg aaccccttga |
| 12721 | aatgatacac acaattttgt acataggtag aaatgtacat ttttctatgg aaagagtaag |
| 12781 | tccgtagctt tcattgaatt ctctgaaaga tcaaggaccc aaaagagttt actgagaaga |
| 12841 | aggccaagag tttactgaga agaagtacac ttggggtact ccaactttac aggacaggca |
| 12901 | gagaaaaaaa gactgaaaag gaacagtcag agaggtggga tgaagacgag cacactgtag |
| 12961 | tttacgccaa aacagttaca tcattattgc agagccagca taagctacag gacaatttga |
| 13021 | tttagggata atacaaggag aaacaagtta ggatggagac agaaaacaga caggacttgt |
| 13081 | cagaaacagt gttattaatg ccttaaaaac agtaataacc cctactacag cagtgacatg |
| 13141 | ggaaatacaa ataggtagaa tatcctggat tacaatttcc caaaggaaaa ctaggcatct |
| 13201 | gagagtttaa gtgcattttg catgggttga ccactgtcta gcaccatatt ctcttctctt |
| 13261 | ccaccaccaa gagcccccag cttatgctaa ctctcatgac aaacacaatt ttagtacctc |
| 13321 | tcactaccaa ctatccagga accaggaatc acctattact acRgttccag cagaatggga |
| 13381 | atcccattct cggatatcca gggtaaatcc ctgaccatgt gagaggaatc ctagtgcccc |
| 13441 | aacaacctca cccSctgact cctcctcaaa ggctctgcca agtcaacaaa aaaatcctct |
| 13501 | acatttacac tatctgtaaa gccaaagacc agcgtcaacY taaatgtcca tcaataaggg |
| 13561 | aatggttgga taagtaaaaa ttatgcagct gtaggaagga atgaagaatg tctatatatt |
| 13621 | cctccagatt aatctctaag atattcagag aaaacaagca agatggagaa aacatatata |
| 13681 | cttgttacca tttttcatgg attcatatat acacacacat gtttacattt aaacaaaatg |
| 13741 | gaaagacaaa tcatattttt tagtggttgc ctatatgcag agtgaaggaa tagtgtggaa |
| 13801 | gaggtaagta gagagctaca tttctttgaa tataatgttt ctttataaat ttgattttag |
| 13861 | aaccaataaa tattttacac agtcataaaa caaaatttaa atagcaatat gtccctaaaa |
| 13921 | aaMacaaaag aaaaatgagg caaatgaatt tggttatgta tccaattggt agcataacca |
| 13981 | cacagagaga aactattcca tctgactata aaacaattaa tttgacaata catccttagt |
| 14041 | aggacatacc ttaaggtcat acataaatgc agatctgcat atatatgtgt gtgtgtgtgt |
| 14101 | aatgttcagc aattatatta ttggtttaaa tattggaatc aatattctga gactgcttta |
| 14161 | tgtgcatcaa atgagtaatt atgttggtat tattgaagag caagattttg taacatggtt |
| 14221 | gaaagaaaat agaaatgtag gccaatgaag ttaagtcaaa agtcagtagt tggttctaag |
| 14281 | tatgaactgg atgcatcagt ataaattcat ggtgtagtgt atctttaaaa caagcaaaaa |
| 14341 | atattttcta cctctgtcca cagaaagtgc tccaaaacaa tgactaacag caaaattagc |
| 14401 | acctctagca cccatattat gttctctaaa gaccattgcc cactaaaagg aattaggagt |
| 14461 | cctgggagaa ataagtgatt ccaggtctgg agcataaaat gtataaRtat atttacatat |
| 14521 | tacaaagtaa ggaagctatc aaatactatt aggattatgt caaaaggact caggatccaa |
| 14581 | cctgaatatg ttaccactgg tcaaagatgg gacaatttta cctataatgg attataacaa |
| 14641 | ataaaatatg tttaaaatct taagttcata aacatatttt ttaaaacctt agtcaccttc |
| 14701 | agaagatgct agagaaacat cacactatat ttttatttat ttatttgaaa taattttaaa |
| 14761 | cttagagaaa agtgcagaaa ttatgcagag aattgtactc actgtgccct aatattaaca |
| 14821 | tattatatta ctatagcaga attacgaaaa tcagaaaatt accattggtg taatactgtt |
| 14881 | aactaatcta cagactttat tttaatttct ccagttttcc cacctatgtc tctttttttt |
| 14941 | tttttttttt tttctgagac agagtctcac tctgttgccc aaactggagt gcagtggtgc |
| 15001 | catctcggct cactgcagcc tccttctccc gggctcaagc aattctcctg cctcagcgtc |
| 15061 | ctgagtaact gggattacag gcatgtgcca ccatgcctgg ctaatttttg tatttttagt |
| 15121 | agagacaggg cttcaccaag ttggccaggc tgttctccag cgcctgacct caggtaatcc |
| 15181 | gcccgcctca gcctcccaaa gtgctgggat tacaggcgtg agccaccacg cccggccccc |
| 15241 | aacctatgtc tctttaccag ggatccaatc caagatccct cattgcattt aattgttgtg |
| 15301 | ttttctcagt atccttcaat ctgtgacagt tcctcagttt ctttgtcttt catgagcttg |
| 15361 | acactttcaa aaaatccaag ccagtgattt tgtaaatgtc actcaatttc agttggatct |
| 15421 | tttctcatga taagatccac atatgcatac attttttagc aagaatacca ctaaagtaat |
| 15481 | gttgtgccct tcttggcgca tcatatcggg ggaaattgag gccagtacgt cttattactt |
| 15541 | gtaatgttat ctgtgattat atagttaagg tagtgtctgc catttttttc actgtaaagt |
| 15601 | tactgttttt ccctttgtaa tatactaata agtaatattg ttggggagat acttggagat |
| 15661 | tatctcctca acaagagaaa taggatattt gagattatgt aaatatccta cttctcatca |
| 15721 | tacttttgcc cactaatttt agcatcaatt gacagttcgt ccttataacc ataactgtga |
| 15781 | agtgtgccca atgattattt cccctttccc tctacattta ctcattggaa ttctgctcta |
| 15841 | aggaagagct taccttctcc tctgtttact tatttattca atttttatat tagtatagat |
| 15901 | atttattttt tctttaggtt ataacctaat aatgtcatta tttttttgct caagttttcc |
| 15961 | tagatttggt cattacgggt tccttcaagt tgcttcctgt gtcctctaga cgcatgaaaa |
| 16021 | tatcctttca ttttttgaac acttccttac tttctgacac cacataatcc tccaggctta |
| 16081 | tctggcatat tttgcatccc agccctggaa tcagccattt ctctaaggat ccatggttct |
| 16141 | ttttatgaga gaatggtgtt tagaaaccaa ttccttgatg ccatgtgtct tcattgcttc |
| 16201 | tgtgttgtca ttgcttctag gccctcccag ccaacacagc taggaaataa atgtatgtgt |
| 16261 | aggtacacat gcatacatct ctacttattt ctgtctatta atatatgtgt acacatatgg |
| 16321 | ctagatataa tatgtataaa ctatatataa cacattgtat gtaaaatcat gaattcatag |
| 16381 | tgatacccac tattccaaac caatgccttc aatctagcct tccccccttt atttggttat |
| 16441 | tctttttctg actttgagaa actagactct cattagtcac aatacacagt cattagttca |
| 16501 | aatctagcat acgtagtttc agaattacta attcatatac ctatgaaaaK aaattaacta |
| 16561 | gagtacagta tttgtggtgc agttatttct gtccttagct ttacaacaca cagtcaaaat |
| 16621 | actgttttcc aaagttactt aggttattct tttcttcctc actcttcggt atagttacat |
| 16681 | tatctattta cgttatactg tttatttgtt actgttttta tttcatttta gtttttccct |
| 16741 | acatcttggt tgattttaag tatttatcta tttgggaggt atgtaaaacc ttatgatggt |
| 16801 | tgtaagagtc aaagctatac aagaaagtat tctcaaagaa gtgttgtttc tcccttatcc |
| 16861 | ctactatccc attttcattt tcccattttt ctctacccca ttctcccaga cccgtgtagg |
| 16921 | taaccagtct cattagcatc ctgtttattc ttcttatatt tcttttacac aagtgagcaa |
| 16981 | atgcatgtag gttttcttat gtctcttttt tcttacaata aggtcagcat actatagata |
| 17041 | ttctatttta ctttgctttt ttacttaata tattctggaa atcactccaa atcaattcat |
| 17101 | agacatctgc cttattcttt tttacagctg tatagtactc cactgtatgg ctgtgccatt |
| 17161 | gtttattcaa caactactct tccatatatg gatatttagg ttgtttccaa tattctgtag |
| 17221 | taatgaacaa tgctaccaca aataacctta tgcattccat catgttgcag gtatatcttc |
| 17281 | aaggtagatt cctgtaggtg agaatgctta gtcaaaagtt aagtgcatat tagctctgtt |
| 17341 | aggtattgtc aaatttccct ccccagggtt gtactaattt gtattcccac caacaatgta |
| 17401 | tgaaggcacc atttttccca tagcttcatc aacaggatat attgtcatac ttttcaactt |
| 17461 | ttgcccatct gataagtaag aaatgatatc tcagctttga gtttctttgt gagtgcattt |
| 17521 | gaactttttt ttttttttgc atctttaagg gtcattttta tatcttttgt gggttgtctt |
| 17581 | tcatgtattt ttctcatttc ctatcaggtt tctggtcctt tgtcctcaat ttttaacact |
| 17641 | Yctttttata tagggaatta gcccttaaac tgtggtacat gctgccaaaa tttcctccca |
| 17701 | gtttgtcagt tgtcttttga ctttatcatg tttttgacta tgcaaaatgt ttatgtagtc |
| 17761 | agatatacca acattttatt taattgcttc tggattttga gtcacagttt gaaaaccttt |
| 17821 | tcctgtattt agattaaaga ggaatttact tgtgctttct tctagtacta gcgtggtttt |
| 17881 | actttgtaca tttagatccc caatgtattt ggtgattatt tttatgtatg gtgtaagata |
| 17941 | taaatctaat tttgtctttt ctcaaatggc taccccattg tcccagcacc attcatttaa |
| 18001 | aagtctgtct ctgacccagt aattttagat gtgaccctta ccctatactg tatttccata |
| 18061 | tgtagttgga tctatttatg ggctttctat tctatctcag ctggtctatt tctctcctca |
| 18121 | tgcatcagtg ccacactgtt ttatctatag aggctttaca gtatgtttta atatctggta |
| 18181 | gggctagttt ccatcacagt ttgtttatca gtattttctt gcctattttt gcatgtttgt |
| 18241 | ttcttcatat gaacttaagt atcgatttgt ctaactccat aaaaaaagtt tgttgatatt |
| 18301 | tttattggac ctgagataaa tttataaatt aactcaggaa gaactgacat ctttataatg |
| 18361 | tcaagtcatt ctatccaaga tcataggatg cctttcaatt tgttctttgt gtttttgaaa |
| 18421 | agtgtttaaa agttttgttt atataggttt tgcacatttg ttgttaaata tatccctaag |
| 18481 | tatttcttct ttgttgctat tataaatgga attatgttct ctaactggtt attgtttgta |
| 18541 | tatgagaagg ctgttaatta tgtgtgttag ttttataatc tgttaccttt tatgtttgag |
| 18601 | ttagcttatc attgattctc taaggttttc cagatatact atcatataat acaaaaatag |
| 18661 | agttttatta ctttgttatc cattcttaca tctctaatta aattctcctg tctaattgca |
| 18721 | ttaggtaatg tctctagaat agtgtcgaat catagttata gagatagtga gcatcctccc |
| 18781 | ctggttccta atcttagtgg aattgtgcct agtgttttcc tcattaaata agatactggc |
| 18841 | tttagttcta aggtacaaat actttatcat gttaagaaga tatcccacaa tcactatttt |
| 18901 | taRtgttttt atcataaata gatattttaa ccacactgtt tttttaaaac tgacaaaggg |
| 18961 | aaagaatcaa gcattttttt cctgccattt ctatatgatc tacacctgaa ggtaaccaaa |
| 19021 | tgaaagattg ggcaagttta tcttcctgga attactccag caataaacaa aaaagaaatt |
| 19081 | atccaataag aatatcacta ttttgcaacc cctaacaaaa tagtcaatcc agtcaaaggt |
| 19141 | catcaataga tgctaccttt acaaaaagag aaacatcagg cattatacat cctgatgaaa |
| 19201 | gtgtacacca ccatctatga agtagtcttg ccaaaagaga gagagagaga gagagatgga |
| 19261 | gacagagttg gtgagaggag agagagagat aaagacagac acagaaaaag agacagacaa |
| 19321 | tctgaatcaa gcctctagcc ctaacaattg acaagaaatt caagagacag aggaaaatgt |
| 19381 | cagacagcac agagRtatac tcagtcaaat acagtcatga gaacttgaac aaatgatctg |
| 19441 | atttcttcaa caaagaattt aagggaggaa gaaagagaat aagatggagg gaaaagtcta |
| 19501 | tgtgttattt aaagaagcta tatgtaWgtt tatatgcacc tacagatatt taaataaact |
| 19561 | tcaaagacat atcaactaat tgcaatgtat gggccttatt tgaatcccaa ttcaaataaa |
| 19621 | caggctgtaa tagaaacatt tatgagacaa ctgaggaaat atgagacgac tggatgtttg |
| 19681 | attgtactaa ggaattattg attttttagc tatgtaatat tttggttatt taggtataaa |
| 19741 | agtaagtcct tatccttttg agatacatat gaagtagtta caggtaaaaa tgatattatg |
| 19801 | acggagacta tggagaggga ggtcatggta tttatgaaat aagattggac gtgtactgat |
| 19861 | ctttgtcgaa gctggtgatg gtttacatca attctattat tctctctgcc tttgtatatg |
| 19921 | tttgaaattg cccataataa aaaatgttct tgaaaatttt gcacttatta tatttaacat |
| 19981 | ttagaccaaa tgagatcata tactgtggaa tgtggtggaa acaatcaaat acatccctgc |
| 20041 | cataagcagt gttgaccact gtcagtctct gagccctcaa gggctaatgg ccaggttgct |
| 20101 | cccaagtgcc agggtaacag gccccagggg cctagtgaca agagtccttc cttcctcttc |
| 20161 | acccctcaaa ggaccccagc tctgtggatt tctccctccc acagggaaag ccccaaactc |
| 20221 | acatcattct ctgaggtccc acagaggata caggatttgc actctatgca ctgccacttg |
| 20281 | taggtcttga cagcctcggt catgttcagg gtaaactgca ggcaggttgg gtgacctgga |
| 20341 | ggaagccaga aacaacagca ttagggccRa agtatgaact gccacccctc aacaccactc |
| 20401 | actggaaaca cattgatgcc cagtgtgcag gcagggcagg gtagcatgac gtagggaaaa |
| 20461 | aaatggggac ccaaacaggg ccctacccac attcttcacc acccMgttat tcagatcaag |
| 20521 | ggcaaaacag agatataagc acatccaatt ctgcttgcac tRagaccata ttccccagca |
| 20581 | ggtgtaacca gttgatgaag atggaaaaca atgatcagag agaacgagag aggaaactgc |
| 20641 | agagtacaca gcctggggca gcactatgag agacaccagt gggaccctcc aagccccagc |
| 20701 | atcagctatg gcagcacaac aggtaagaac tatttggagc tgggccttct gatccaacca |
| 20761 | gttattcaca ctgccttagg gtgcaaggga ggctatcaga aggattgtat gaggtttcca |
| 20821 | gggtcagtgc tcagggatag gatgaggcat tgcactgtta cctaaagtat ccatgcccac |
| 20881 | actcatgaga ctagcaaaag ttcataWcct gaacagtacc acggactaca gaaggaagtg |
| 20941 | gcctggcaRg gatggcaaag aaatggagca tcagagcaaa tggccaggtc tatcctaaaa |
| 21001 | ggactggttc ccggttctaa ctcttttctc catcccaagt ttcatgggag ccaacccaat |
| 21061 | cacaagatga gagaagggac attaagctgt gaccttcaag cacagccccc gtgagccatg |
| 21121 | cccaagactt ggcctgtgga actggactct gtcctcagac ctaggtcacc taaaagagag |
| 21181 | atgcttgcag ggagaggacg gccatgaact gtagaatgca aagacccttg gccttctatg |
| 21241 | tggcatggaa ggataacttg gatgacattc ccaagtcKcc cagtactgtg aaacagatca |
| 21301 | gaatctgccc agaRcaccca gggcaaagcc aattactgac tctgtcacaa aaaacattga |
| 21361 | cagctttgcc cagtgccgag cactcaccgt tctccagagc ttccagttct gccaaaccca |
| 21421 | ttatatggtg ccttgtgttg cctctttcat ttgaaggatt atatgcttta aagaaggatg |
| 21481 | ctagagacag gggacaccaa tcatggttag caccgccaac ctagtatcct ccacagacag |
| 21541 | gatcccccat ggcgccacca ccccttggca aagccacatc ttaacaggag cccccatttc |
| 21601 | ctccctttca aatcttcaga gagctgttca catcatgacc atttggatga aatactgtct |
| 21661 | attcagccaa cataaggatc cttcacattt tgagctccac aaatagaaac catgctgagt |
| 21721 | tatcttggag agggcgaggg gtgtagaaat gacttgttgg tggtggggag gggacactca |
| 21781 | gtgaatacag gcagaagcag caaggagagg cctgatctaa aagcttttca aggacttctc |
| 21841 | aggtaccatc caacttagag acttccgcag ctctgaacta ataaccctct caaggtgata |
| 21901 | ccatSacagt ggccccaaag aaaaagaagc cttacattaa aagcctgtgt gaacagccag |
| 21961 | ctgatattct tgaaagcatc tcaaagattg gtctaagaag aacattagcc agggttactc |
| 22021 | cctattccag tctctcagag agaacactgg gatgggtaca gggtaggaaa tatctgtttg |
| 22081 | ggactccatg tggtatgacc acagatgccc agtttctctg accaggcacc actgagagca |
| 22141 | tgaacatgag gtcatggagc tgatgctcat ataggcaagt tgactttggc cagttctgtg |
| 22201 | ccacacccca gccacaccca gccagcttag gataactatt cattaactta cSaagtactt |
| 22261 | attgagcacc tacaaggcag gccattgtgc tagacagaaa agacacagtg gggatgagca |
| 22321 | agataccttg agcccttcca gcctagccaa agaggcctca gattaatcag ctacacatgt |
| 22381 | atctatcaac tacaattgta taattagtgc catgaaggaa aagcataatt ggccgtaaaa |
| 22441 | acacatagaa gaacctaacc tagactgaga tgacaaggaa accttccctg agaaagatct |
| 22501 | ttaagctgat atgtgtcaga tgagacgtta gctaggtggg aagaggagga ttccaagtaa |
| 22561 | ggggaacagc atgggtggag gctttgagca gtgaagcagc ttagcaaatt acaaggaaga |
| 22621 | gaaaggccag tgagcagagg cttgatgagc aaaaggaaag gggtaagagg tagctgcaga |
| 22681 | ggtagctgga gaaggattac aggcatttgc agcccttatt taagaatctg ttttttaagt |
| 22741 | cattaaaggc tattaagcaa ggcagtaagt gggtcacatt ttgtttttaa aggagcactc |
| 22801 | tttagcatga gtgctattgg gatgaagtgg ttctgtatct tgattgtggt ggttacatat |
| 22861 | gtgataagat tgcatagaac tgtacataca catacacata tgtatttgta catgtaaaac |
| 22921 | tagtcacatc tgaataaagt Ytatgattgc attaatatca attgcctggt tttgatattg |
| 22981 | tgctataatt acataagatg ttgcccttgg agaaaactga gtgacgagct caaggacttt |
| 23041 | ttgtaatata ttttgtaact tcctatgaag ctataattat gtcaaaataa atagttgaaa |
| 23101 | atatattttt aaataaggaa aacaaataaa aaattaaatc atcaaaaaga aaggaaggtg |
| 23161 | gaaggcagat aagaaaaagg agagagagag gaaggaggga aggaagaaag gaaggaagga |
| 23221 | aggaaagaag caggaggaag ggagggaagg agggagggaa ggaaagaagg aaggaaggag |
| 23281 | aagggaagag atgaaaggga cagaagggaa ggaagggaag gaagggaaag aagggaagga |
| 23341 | gggaaggatg gagggagggg aaggggagag aagaggggaa ggggatggga aggagagagg |
| 23401 | aagaggaggg gaagggaggg gaaggggagg ggaaagggag aggggagggg gaaggacaag |
| 23461 | agaaggggag gggaagggag gaaggaagga tgaaggggct gtacgctaag gaaagaattg |
| 23521 | ttgaggggtg ggagggtaca gSaatgtaag aattgttgcc aggaagctag ttaggaggct |
| 23581 | aatgcagcta ccaggagaga tacaagaatg attcaactaa ggtggaaatt tgagaaatgt |
| 23641 | taaagaaata cttgacagga cttgactatt gtttgaatgt tgggaatatg ggaaaatgat |
| 23701 | gagaaataat cccaagttct gacccatgaa actaggtaga ttgtagatta cagtgaaact |
| 23761 | tagatgagaa agactgtaga aggattgcag ggaccacata gaggaagagg attccttctg |
| 23821 | tgttgagcat gctgaaactg agaattctgg tggaaatgtt gagtggatag ttgaatgtat |
| 23881 | gggtcgaaat tcagaagttt ttgctaaata ggtgataagt gaaaccatga gaattaatga |
| 23941 | aattatctag ggaaaaggga atgaggaagt atctagggaa agaaaggaag aggagagaag |
| 24001 | aaaggagaat agagaaaagg gtctaggact gagcttggag gagctcaagt cctccaagat |
| 24061 | caaaagaaag aaccagcaaa ggaaagagag aaacatggca agaggtaaga agcaaatcgg |
| 24121 | gtgagtatgg ggtcacagta accaaaagaa gaggtttcaa gaaggagaag gtggtcaact |
| 24181 | gtgtttactg ccacaggttc tctagtttgt ccttgctcaa taaagacccc gagtgaggga |
| 24241 | atgttgacag atcagggcaa aagctaccct actaataata acacgcttca gtcatgccat |
| 24301 | cttcccaagg aaggaaagtc agcacattcc agagcatgac agaactcagc agccaaatgt |
| 24361 | gcatctgagc tgtctctgtg aggaggggcc gggggtggcg tgtgctgcat gcatgtagca |
| 24421 | acactccacc aatatttatc gagagccagc tctgtgtctt agttctctaa gactcctgtg |
| 24481 | ctgagctatg aagacatttt ctgggatagc tttggacaat ggatggtagc aagtgggttt |
| 24541 | tggaaatgcc tgttggaagt ttctgaaaca atctcatggc tttctctgtg ttcccatctt |
| 24601 | gagaaagtca tcagcctcct gaagatcttc aaacccgaag caagggagct aagttcacac |
| 24661 | agcctcaagc ccccgaYaaa atgcaggact aagatgggcc ttagcagtgg ctgcaggaac |
| 24721 | tccttctcct ccacactcag gacatcactc aagcgcaaga agaaagtagg ggagggaaat |
| 24781 | actattgtaa cttctcagcc agggagggtg agacacaaaa agtttaagtc atcagcaggt |
| 24841 | gcaaaaactg tgcccagaac taaatcctcc tgtctggacc accataacag atttgctcct |
| 24901 | gcgcctttct ccaaaaactg cagaaatcat ttgccagaca aaactgcaag aatgagactt |
| 24961 | cttacctgcc acaaaaatta cttcaataat acacactgtt agacaaaaRg aagtgtctaa |
| 25021 | ggaaagtaat agcagcttgt ttctaaggtt cactggtctt acttttgccc agaactaatt |
| 25081 | cctaaatgct ttgaaagtaa gagaataaca aatacggtct ttccaattca aaggtgaaag |
| 25141 | aaaaaaagag gcacatggct ataaggtaat ttgtaatact tatggtcact tgatatttga |
| 25201 | ggatccctat cttaatctct tttgtaaagt taaccctcat tctctagggt attaggctgg |
| 25261 | tgcaaaagta attgcagttg ttgccattaa tggcaagtac tgcaattact tttgcaccaa |
| 25321 | cctaatactt gcttggaaat ctagattttt tcatagcccc agttttcttt agagcaagaa |
| 25381 | atacccatat tgctatttaa atagcctact atagtggagg gaagacaacc cttcccaggc |
| 25441 | ccccctgggt gccatgattc aacagagcat ttataagatt ccaattacca aaatgaattt |
| 25501 | agaaaatggt gcactgtgca tgattcagcc cagccggatc attaaaaacc tagtccttcc |
| 25561 | cggatgttcc tctaaatgta ttctatcagg agcattcagt aaatctcctg ctagcttRtt |
| 25621 | gagctgggtc agtggtgaac ttggtgtgaa gagccattcc tgattcagcc ctaacccttc |
| 25681 | cagaactgac ctcaatcctc cctagaggtt gacactgggt atgtcaccca tggccactat |
| 25741 | gtatgggaga tctaactgct ggagtgggct tttgggagat cagcatttgg cttcagggtg |
| 25801 | ctatttcagc agatggaggg aaagggaaac caatgggtta tgggaaactt ctcagacaga |
| 25861 | tgataatcca tgaggctggg agtacccggt gtttctgaca ctacagccta ctgagctcag |
| 25921 | taaaaaagga aaggcaggca tctgagagca gtgacagcag ccttggcact ggaaggacac |
| 25981 | atctcatgtc tttacagaaa catgtgcaca gatgctgaaa atgttcaagg gacagcaaat |
| 26041 | gggatttgtg gtatttgagt gagaaggcac acagataaat aYccaggcaa aactagtgtt |
| 26101 | agtttcatat tcacagctgc caatttctat gcacgMtaat acacaactgc cccaacacaa |
| 26161 | gggtatttcc agccttttag gggagaaaat atctaaatat accttgtgga ttaagctgac |
| 26221 | attatgattg caacttaaaa gctaacctaa gtcacttact agtcaacctc ctctatagat |
| 26281 | attcagtaca ttgagagaaa cacagttcat tttcaacaaa ccctgggcaa aagaaataca |
| 26341 | caaactgcta tgacatgtgt ctcttacatt atgatcctta ttaaaagagt ttttatttta |
| 26401 | ttatcaccgg tatttaattt ggactaggct aagccacatc actgattgaa ttagggctcc |
| 26461 | aggaaaaaaa attatcttcc taggtatgca Ytgaaaacat aaataaaaca aatataaata |
| 26521 | actttggaat cagcatggtc tattatttat tcattgtgaa attataaaca tctaattata |
| 26581 | ctttattacc ttatttatca atgttgatct cttaaaagct aatttctcag gcaaagagaa |
| 26641 | cttaaatatc catggttctc aggcaagtaa atgcaaaact caaattgtgc tccttattct |
| 26701 | gatgcttggt ttacagaaat tgtattacat aaaatacata gaatcaattc caacccaaaa |
| 26761 | tagcgccaaa aacaagtctc catgggcatt cattagaaaa taatttggct caaatttcaa |
| 26821 | atagttctct atattctcat acacatataa aattcctggt atttacaaat tgcaaatgta |
| 26881 | tatatgaagt tttagctaaa gcatttatgc aaagtatagt catatctgaa tcatcatgct |
| 26941 | acagggtaat ttgagaagaa aaattattac tagttctctt tgggaagcca tctcattcaa |
| 27001 | tcagatccat tcattttcat tctctcactc ctctcttctc tctttctctt ctctccctct |
| 27061 | tctgtctctc tctctgcccc attccctccc cctctcttca tgctcctcca ctttgagacc |
| 27121 | cagcttggca ggtggagaga aggaggctca gtcattatat tgaatctgta aaggggaaag |
| 27181 | ggatggacca attctgagca atcaaaagtt caactccttt atgtatatac tatggcaagt |
| 27241 | atgataactg ggcactgctg cttcacataa ttatttctca agccctgctt tgtaaacctt |
| 27301 | ctgcaaaggt ctgcaggttc acctctatct gcccccagat gttacttcta atgctgaaga |
| 27361 | cacctgcaaa tcgatgatca caatggcact ccagaggcat ctgaatggct ccctttttct |
| 27421 | ctcaccattt ttattgcact tcaaagagga caaagaagag aaagagaatg gaaaggacaa |
| 27481 | ataccaaaca aacaaaaatg cattaaaaat aaatgatagc gtgagctgtg tggctgccct |
| 27541 | ttcttgggca aaacaccttc ctgtgcatcc agctccactc ctcttagagg tttgtttatc |
| 27601 | ctctcctcca gttgtcaagg tctgtggcac ccaggaaaca catgggatgc aagccccagg |
| 27661 | ggctattgca ccagtcattc ttataaataa ccatacaaag cacattcatg caagagtatt |
| 27721 | acgggcactt ttaataggct aaacagccaa gggctcatgg cgccattctc aaatggtcct |
| 27781 | agaaattccc caagttttcg tggtcaggtg tggtccaagg tggagtctct aggaagtgga |
| 27841 | tctacacctg tctataagcc tgagggcata catcatggtt ggtcttctta aagtgaaata |
| 27901 | atgggatagg gaagccttga cctggattta tacaatttac tcgactcaac ttggggccaa |
| 27961 | tgcacagcac taggaatacg ttgagagtca tagtaatgac cccaaagccc ctaattagtc |
| 28021 | actgcagctg aggatcaaaa ataatttatt catttattca ttcctttgac agtatttgtt |
| 28081 | gaatatataa atatggcagg cactcttcca ggtactgggg atatagcagt gagaaaatcc |
| 28141 | aaacccctgc cctcaattta caactgccac actttgagat atattacagc acctgagtcc |
| 28201 | tttggacaag tcaattgact tctctagcat tagtttttca atatattaag ttaaagcatt |
| 28261 | ggaatctgct atccatgaag ctgtagcttt gtctatgcca cccaaaaatg gtatttttaa |
| 28321 | agatactact gatccccaat tgcccatctg cttggtggga taaacaagcc ttctaaagtt |
| 28381 | tgccagctaa aactgaaagt taaaaaagaa aaaaactaag aaagaaatta aaatagccag |
| 28441 | taagtccttc tagaattcat gtcaactttc ccatattgac cagcccagtc cgatccaatg |
| 28501 | tcgacacaac aaaagatggc caagacagaa agcaaaagaa atctagaaat ctaaaggact |
| 28561 | tttcatcaac cataccaccc accgccatgc aataagttag gcctatacag atgagctgtc |
| 28621 | ataaactgaa ggtagacaga ggccttgggt aaggttccat cgatgaggaa tattagatct |
| 28681 | gggatgagct aggttgagct gctgtgtgac agctgcagat attgccaggc aggaagtgac |
| 28741 | taagagaaga gggtcatcgg tatgagcaac tctggatgac tttcctttta aatgcttctc |
| 28801 | tggatttgta gattgcaggc aaccagctgc ccaaagtgat tcttgcccca caacagtgtg |
| 28861 | tgaaggtcag gcNgctgaat cattgYccag agcccaacat caattttaga tgccaagcct |
| 28921 | tgacatttgc tgtgcattcc aggcctctct ggacctcagg agcatccaac catgggacct |
| 28981 | ggctaaaggc tctgtgtttt ctttaggaaa atgagcctga aaaagaaacc attgcttaga |
| 29041 | ttggaaaccc tgtaatccct gtacttccta ctgtcctgag tacatccagg agaatttcaa |
| 29101 | aatgNNtgta attgatgtat agctggtgga aaacagcagt gattggtcaa cacttgattg |
| 29161 | cccatgatcc tccaactgct gatcatccat gaattagaag atgaattcat gagtggtcta |
| 29221 | agttcttgag ttgcctgagg acctttgaag tagcattcta attttttctt ccaaaaaata |
| 29281 | ttccacagag ttgtgctaag tcaaaaggaa aaaatgcttt cctatccttt tctacgtgag |
| 29341 | cctgggaagc aatcatagaa gtacctagaa gtagaggact gacagtgtac tcatgacact |
| 29401 | tagacacttt gttttacatt tttttttttt tttgagacgg agtcttgatc tgtcgtccat |
| 29461 | gctggagtgc agtggcgtga tcttggctca ctgcgagctc tgcctcctgg attgacgcca |
| 29521 | ttctcctgcc tcagcctcct gagtagctgg gactacaggc gcccgccacc aYgcctggct |
| 29581 | aattttttgt attttttagt agagacgggg tttcaccgtg ttagccagga tggtctcgat |
| 29641 | ctcctaacct tgcgatctgc ctgccttggc ctcccaaagt gctgggatta caggcatgag |
| 29701 | ccagtgcgcc atttttaaat cattttagtc taattttgag tgttcttgtg gactttgcaa |
| 29761 | tctacccaat tgtgatcaga ttctcataga aaaataattc cacttaattt ggcaaaattc |
| 29821 | actgagcacc tgttcattgc caggaactgt acaaggtgct gaagtttcaa agaggaatga |
| 29881 | ggcatgactc ctgccttgag gagctcagtt tggtaggtgt gctaggcagt ctctggcgtg |
| 29941 | gctatcaatg aaggacacca tactcttttg tcattctccc cccttgagtg tgggctgcac |
| 30001 | taaatgactc acctcaaact agtagaatat ggcagaagtg atgagatgtc acttataata |
| 30061 | ttaggttata aaaagactgc aactttcatc ttccgaatcc tctcttatgc tttctcactt |
| 30121 | acttgctcta agggaagcca gctgctatat tgtgagatgc cctacagaca ggtccatgtg |
| 30181 | gcaacatatg aaggaaggcc tcagtgcagc agcccaaggg gaactgaatc ctgccagaac |
| 30241 | ccacacgtgt gggcttggaa ttgaattatc tcccaagtga gccttcagat gagaccacag |
| 30301 | ccccagctga caacttgact gcaacccaag ggaggccttg ggccagaggc acccagataa |
| 30361 | aacacacata gattcctgac ccacagaaac tatgagataa taagtgtttg ttgttttaag |
| 30421 | ctgttgagtt ttggagtaat ttcttacaca gataactatt atatatagag agagagactc |
| 30481 | ttttaaatga ctataataca atgtgataag tgccataata ggagaatgaa tgaagttcct |
| 30541 | acagataagg aagcaataaa taatgctgga gaggctcagg gaaagccact gagaggaggt |
| 30601 | agctttcaag ctgggcctta aaggataaat aggagttttY taagtaaaaa aaaaaaaaaa |
| 30661 | aaaaaaaaaa aaaaaaaaat ggaaacagca ttacagggaa aggcaacagc aggaaatata |
| 30721 | aggtgcatgg tccactcagg caaagcagag tagctcaagg agagcaggga aaagaggaat |
| 30781 | tgcaggagat gatgctagaa Rggtatgagg aaagattatg aagagcctct tatgtaggca |
| 30841 | tgaggatctg aatttaattc tgtaaacagt ggggactcct cggaggtttt taagcaagaa |
| 30901 | actgatgtgc ttgtacccat gctttagaga gatgagtctg gtgataggtc agaaggcaga |
| 30961 | tRtgttggag atggctgggg aagggggacc agtcactaaa tccatctatt caatgagttt |
| 31021 | gtattgagca cttgcaatgt gccaggtgct gtgatagatg ctgagcaatg gaaaacagac |
| 31081 | aaaatcctac ccttctggag cttacactgt aatgaggggc attagtccag ggcagatcta |
| 31141 | ataattgtgg ggatgaaagg aggaaacatt tgagaggtag aattggtcaa taggacgtga |
| 31201 | tacctgcttg aatatgtgtg tgaggaagag gcagaagtaa aaaatggcat caagattcct |
| 31261 | aggttgggcc acagagtaga tgataaaact gttagcagag atgcgacttg cagagtataa |
| 31321 | caatggattc aaactgggca cattccatta attgtgcaga tgaagatgcc caggaattgg |
| 31381 | tgtgaaaatg tgggtctaca ttcaggagac aagaccaggc tagagatata aatttagaaa |
| 31441 | tcatcaggtt tgggtggtag atgaagcctt ggaagttggt gagacattca ggaagagacc |
| 31501 | acagagcaca aaataaaaag gacctagtta ggggtagagg gaagagtaac cattggagtt |
| 31561 | gaaagcgtta aagagcacca agaagagagt ccactgagaa agctggagac aatgtggtca |
| 31621 | gaaagggaga agtatctgga gagaggcacc tcatgacatc cRaggaagag gaagggtggc |
| 31681 | accaccatca agtgacagat gaagtaagat aagaaacaaa gagaacgtgt tgccctgggt |
| 31741 | gagccattgg tgatgagaac taacacagtc actagtagca atgggccctc atgcctctcc |
| 31801 | aaatcactga tggcttcagc agcgcagaga gaagaactca tacacagatg gcctcacaac |
| 31861 | atgcttcttc tgccgcaggc tttgtccagc actctcaagc ttggaagcac cgtgcacaat |
| 31921 | gatctcagga acattgtctt tacctaactc cattcactca tagcaggaat cattttctga |
| 31981 | tgtagattct ggggccaaag ctaggccctg ccagcctcag tttctgtcct tcccccggcg |
| 32041 | atgaatcaaa tcccactgga agccgtgcct ttctttctga ccttggatcg aagatcttca |
| 32101 | gtcctaggtg actgagcctt gcccacaagg ctggggcagg tcctgatgga ggcttctccc |
| 32161 | acagccccag agtctgatca agtggcagtg atgaaaattc tccaggaggg aagttggggc |
| 32221 | caaaggtagg aggagcagca ctgggagcca aacggaatgg tgaggtgagg acagaaatgg |
| 32281 | tggcaggcag caatgttggt ggctgcagtg ctttgcaaaa atgcccacaa attccttggt |
| 32341 | cctcctcttt tcaagaagtg gaggctattc ccctcttctt gaatgtagac tgggtttaat |
| 32401 | gacttgcttt taacaaacac agtaaagagg agtgacaata tgcagttttg gagactaagt |
| 32461 | tataaaaggc actgtggttt ctgtcttgac tgtgcactct gtctctatgt ctctgtctgt |
| 32521 | ctccgatcac tcactctgga ggaagccagc tattgtgttg tgagctgcct catggaaagg |
| 32581 | cccacatggc aaggagctga gggaagcctc tgttcagcca gcaaagaact gagactctca |
| 32641 | acgcagcagc tcatgaggac ctgaggccta atggcaacat gagagtgagc ttggaagtga |
| 32701 | ctccttcagc cctgatggag ctttgagatg cctgcagcct tggccaacat ctggactgca |
| 32761 | gcctcatggg acaccctgtg acacaccacc cacccaagcc attcccaggt tcccaaccct |
| 32821 | cagaagctat gagataataa atgtttgcag ttttaagctg ccaagttggg ggataatttg |
| 32881 | ttacacagca atagctaact aatacaatgg caaaggagga acagtaaggg gcctggctga |
| 32941 | acaccagaca tttgacagaa ctcctgctta ggcagtgtct gcattgtgct cccagccaca |
| 33001 | gtgaggccca gcactgggct ggacaatgaa ggagaggagc agggtttggg attttagatc |
| 33061 | atttatctct tccatgctgt tattattttt tttcagtgaa tctcctttaa aagagctaat |
| 33121 | agtgggaaca aagataattt cagtggcaat atgaatgggt gtgacccctc agcatttaca |
| 33181 | gcaaggtgag tgcatctgcc ttcggtttcc tgggtccaac ttgtttccat ggcatggccc |
| 33241 | gaggagactc actgcaccaa agagcctgcc gtcttcccct caggccttgc cctccatgcc |
| 33301 | cctgtctcac tctcaccctg ctctgccttc cttccacctg cctgagcctc tctcacctcc |
| 33361 | aacaagcctc ccctttaggt tccccagaac acaaccccag tgttagaaag tcagaggctt |
| 33421 | cctagaagta agccaaactg atatgagcag aggaagaacc tgcagctagt attctggcct |
| 33481 | aaagaaagcc atgtttggat actttaatga tccttataac ttttccaagg aagcaccttt |
| 33541 | ctattgactg attttgtttt gattccccgt ggggatcctg tgaggtgggc agaacagata |
| 33601 | ggaacattcc tgatttacaa atgaagaagt tgaggctcag aatggctcag gcagcccgca |
| 33661 | agtaagtagt ggagccaggg ctagtggaac atccatgtct tcacattcta atcctttggt |
| 33721 | ctctcactac accatgtcct aggatggcca acccaaaagt ggcaacccaa tggcatttca |
| 33781 | gctccttgtt taagctgcta aatatagcaa cattttctcc ttgcatgcat aagaagtttg |
| 33841 | ccagggcatg atgaaatatg tcactgactc tctccatgcc tcagtttcct ccatttgcac |
| 33901 | cacataaggg tgaactagct gtcccctcag gcccctttaa gcctcaatgc tccatgcctc |
| 33961 | tagcgaaatc cctcaagaag ccagacttgc ttacatgagg cagaggactg agtcctcacc |
| 34021 | atctggcagt gactaatcct ggaaaaccct ggggaagaat tgctcaggag tgagccagtc |
| 34081 | cagcccacac aaaggggcct ggaggctgca ggaagtaggt cagcagccta tggtggtgtt |
| 34141 | actgggcttc ggctcctgac agaagctcct cagcccctgg ctggagtgag gaggggtcat |
| 34201 | gccccccgcc acctcacagt cccactcaga cttgccaggc cagctattcc cacttgcagc |
| 34261 | tgggtgtcca ggtcctccct agggctcccc caagtgaccg cccatcacca gctttccctg |
| 34321 | tgaagcctct gagaggagca caaaagacgg ctggcatcca aagccaattt cacRccacac |
| 34381 | ctccattaca ctgtcagtca agaaaggaca gaagtgttcc ccactcacct tgtctaagaa |
| 34441 | aggaacaaaa gagcacacag gaaggatccc ctttttgtca tcacaagact ataYtacaat |
| 34501 | Sgagacaccc acaaccccac ccccactgga gaccccaacg aagSctcact ggctcctttc |
| 34561 | aatccttttc cccctttcat tctccacctc ccgatttccc caagtcatga attcactttc |
| 34621 | caagttgcag tggcaggaga atggaaaatg aggatgatga tggtgacaac cagctttccc |
| 34681 | tgaagtctct cacccaggtg tcttgagccc tggagacacc tgatgtcctt caacaccctc |
| 34741 | aaaaagaaga aacagcaatt gcactgagcc aacactgcat acgggagaaa catccttgga |
| 34801 | gctccatggg agtacaacaa aacccggcac catctggcgt cgtattacct ctgcatgcat |
| 34861 | ttgcttccaa gccccaacat ccaggcagca ccgggtctgc actcctcctg tatctgtgat |
| 34921 | cactaaaccc aactgataga tctgtttcca tcggccaaat tttcatctaa gattcatcca |
| 34981 | cttttcctga caatcaaaca aatatttatc caacaaattc ctgaatgaga attgatctcg |
| 35041 | ctgcttactt agaaagctag aggggatgat ttttacatgg gctttctttt tttcctctgg |
| 35101 | agaaattaga gagttggaat agtacaagag aaagcaaatc aatggctatt tccagtgacc |
| 35161 | agcatctgtc ttcaggttta atagaaatga tgccctaaga catatagtct atttggccta |
| 35221 | ttcaatacaa aaacacccat caatattaaa gtagtgcagc aggagggcct ttcctggtac |
| 35281 | tttgtaaggg tagacaagag cctcacaagt gttcaaagat tcacgaggcc cttctggata |
| 35341 | agcagtgaca aactccattc cataatcaga gaaatgaaac catcaaccat ctctaaaatg |
| 35401 | gctctggttc acctgagcat taggtgtgag agaattagga atacatcacc agaaatctga |
| 35461 | cctcctttca gggatagcta aatatgtggc acRtatacca ccattctatc ccatacccag |
| 35521 | ggcagacatt actaatcaat tcaacattgt cttccaatct gcatggataa gaccttatga |
| 35581 | tccttttcag cacMgcactc caagtggctg tcactggtct agagtttgca tacaagaaga |
| 35641 | aaccaacttg ccatccctga gctaggcact attaagctga ataggttaaa agtttcatgt |
| 35701 | gagaattaag gactcacatc ttaagcaggt cactggtcag atatataact tctgttttcc |
| 35761 | tttccatgag gcagaaactc tatcacaaca gctaagaccc aaaccaaatg gtggccatgt |
| 35821 | tttgtggttg gactcaagta ttgctacggt cacatacttc tgtcttcaaa gcaaacagag |
| 35881 | atgctaagaa gggatttttt Nttttttttc agagataggg tttcactctg tcacccaggS |
| 35941 | tagactagag tgcagtggtg caatcttggt tcactgcacc tgccgcctcc ttggctcaag |
| 36001 | caatccttcc tcctcagcat ccacgtcccc ctacctgcct cccccagtag ctgggactac |
| 36061 | aggYatgtgc caccatgcct ggctaatttt taaattttat atagMgatgg ggtctcccta |
| 36121 | tgctgcccaa gctggtaaga agggattttt taatgatggY Kaatattttt tctactccac |
| 36181 | cagctagaag gcatcaaccc attcctcctt aagtaattcc tctgtctttc attgcctgca |
| 36241 | gcatgaagta agcttcttag cattgcaagc ctctttgctg tctggccaca gctgcctttt |
| 36301 | taagattYct cttcttttcc caccatggac cccaRcccca ccataccaca gaaaagtatt |
| 36361 | tgccRcatcc cacaccctca acgctcctac accccccacc ccccaaaaaa ccactcttct |
| 36421 | ttttgacttg cacttttgca cacaaaggtc tctcactatc tctcaactcc cattcatggc |
| 36481 | caaacctNtc aaacaggttg tctacactcc ggctacacaa aagtgggccc atcgaccact |
| 36541 | tgcatgagca tcccctgggg gctggttaga aatgcKcacc ccggatctga cctagggact |
| 36601 | gcagttttaa caagatcccc aggtgattcg catgcacagg Rtattttgag atgcacttct |
| 36661 | ctaaactcac tccctttctg ggacacccac tcagtcttca ctctctccac actgacttcc |
| 36721 | ctcctctctt ttccaccaaa aaaactctca tcaaaggagt gaatgtgtcc atgcagatat |
| 36781 | gaataaaaga ataataaact actacttcca ccttttgcct gtcctgggaa cttctattca |
| 36841 | tcctccaagt ccagtccaaa agacctcact tctgtgatgc cttctctacc ctctaacgca |
| 36901 | gataaggaaa ccaaagtcct gggaggatgg gaWatctgct tacagctctg cagcYactaa |
| 36961 | gtggagWcca cctgactcca agcccatgag tgccctcagc atcgccagtg cactgccttt |
| 37021 | acgaagtgca tccagaaaga tgcccacaac ctcagataaa agccctttgg ccactgtcag |
| 37081 | ggacaggact ccaaggctgg aaccagtaaa acaagtctga ctttgctggt ggatgagacc |
| 37141 | ccatggaggc tgctcataaa tcagattccc ttgtttaagt tcttagcccc tgatgactac |
| 37201 | tgcttaccca ggcctctcaa tctccctgaa gacctttctc caYccccttc cttcaaacac |
| 37261 | acatcccagg cacccatctc ttttgccctc cttgcctcca tgcggaattt ttcctttgga |
| 37321 | gaacaaggac ctcagagtgg aaatgtccat cactggtctt ctcatttctc caagccacag |
| 37381 | caatgccatt ttggcgaccc ggctgctctc aaacttcaga gttcagaaga attaccggga |
| 37441 | gagatgtttt aaacatgcag attctcaggc cccaaagctt ctaattcccc tgccctggag |
| 37501 | tgggtaggaa tctgcctgtt tagctagtgc cctgaggggg ctcagatgca gatggccctg |
| 37561 | gacacacttg gagaccctct gctgccagcg tctcctgggg actcttgcac atcagcaagc |
| 37621 | tgctctataa tgcagcgtgt ggaacctcag ccaagtcttc ccaccactct catcctgagt |
| 37681 | cagccttgct caatgagggc tcgaaagcct ttcctgccca cctcatcctt cttctattat |
| 37741 | gaggatcaag accatgggct atgtactact gctttgttaa gctggaaaac tttaaaagaa |
| 37801 | caaaaggcca aggtttacca ctcttctgct ttgttttggc accactaaac ctctacgccc |
| 37861 | attcccagaa attctgtatt tctttcaacc ccatagccca ttgtcgacat acccgtaagt |
| 37921 | gtttcagaca ggcctgtgat tgttgcatgc aaaggacatc aatccatact ggcctgtatc |
| 37981 | caggcacaag ctgtcttccc gatagcaatt ctccttgcca gagacctgtg ggtctcctgc |
| 38041 | ataaacagga ggtcttagct tctctcagca acttgagaca ggcaccatat actctctccc |
| 38101 | caagataaga tcttttagaa gacaaagcat ttagattatt tcctttgtag gattaaaaac |
| 38161 | aaacacaaat gcccattcca gtatttgtag gactgattca actggagtag taggaaagat |
| 38221 | ttaggataca tttaagacac ggtttccatt gtttcactat gaagctttga gtatctttgc |
| 38281 | aacctaatta gattgcacct attaaatgat gactggattc aacagcccYa gaacaagcat |
| 38341 | tacttttgat cattttctat tttagcKtct attcaactat tttaagagga atatacctga |
| 38401 | gccaaaaatc acgaggaggt ggctagcagt agcaagattg gtcctaattt attgagggca |
| 38461 | gtgaggcaga caccaYcaat tctttaatct cgccaggagc tccagtccaY ggacagtacc |
| 38521 | acttctcacc atatctcctc tcacgtcgtc actgctcatg cacctatctt agattccaca |
| 38581 | tccagccttg cacgtgtctg cctacccctc tacccctttt tcctacttac tcaacttcca |
| 38641 | gttaatacca actcttcacc tactcttggc ctataccaag cacagaaaaa tacaccactg |
| 38701 | tgctgagcag tcgtggtttc agtggtgacc acagatcttg tcattgtccc cgtatttcac |
| 38761 | tagtcgaatt attctcccat ctctctctgt cttttcaaat ctcccatatc ttatacccca |
| 38821 | tcctcacact ctccttattt agttgaaaaa tacataaaca atcagaataa aactattgcg |
| 38881 | ccaggcgtgg aggctcacgc ctctaatccc agcattttga gaggtcgagg caggcagatc |
| 38941 | acttgaggtc aggagttcga gaccagcttg gccaacatgg tgaaacccca tctccactaa |
| 39001 | aaatacaaaa ttagccaggt gtggttgtgc atgcctgtaa tcccagctac tcgggaggct |
| 39061 | gaggtgggag gattgcttga aaccaggagg cagaggttgc agtgagctga gatcatgcca |
| 39121 | ctgcacactc cagcctgggt gacagagtga gactctgtct caaaaaagaa aaaaaaaaaa |
| 39181 | aaaaaagaat aaaacgatcg catcttctcc atgccacacc cacccagctc cccgcctctg |
| 39241 | tatccctacg cttgtctccc tcctattacc gggaatgaac ggtctcagct cctaacccaa |
| 39301 | agccgactgg caccaggatc cctacctctc accctgagaa ttttgctcct gctgttattc |
| 39361 | cctcttgcat cgtcaatgtc tctctctctc tctagYagac cattcagcca gcatacaaac |
| 39421 | aagccataaa agctcccatc ttccaaagag atccttcctg accaagcatc cccctctgtt |
| 39481 | gagcgtccaa tttctctgct ccttcttctt ttagcacaac tggaaagggc cagttctctc |
| 39541 | catgcccctc ctttccacac tctcccctct ccacacactc gcctagctct cctcccacct |
| 39601 | cgctggctgc cccttcttgg tcttctttgc tgaatcctcc tcttctccct ttctcccagt |
| 39661 | tcttcctttt atgtggactc acttcccctc aatgtcttct atMcacttgt tattcccaat |
| 39721 | ttatatctcc agcatgaacc tctttctgaa ttccagactc atatatccac ttgtcatggt |
| 39781 | ggttgggaac ccgaagaata Raaagttaaa aagacctaag ttaggaaagg attttgtggg |
| 39841 | aaaatcctca tcccaagggg tgatgagtac cctgccagcc aggaggggag ccttcagctg |
| 39901 | gggggctgtg gagtctttat ggaggcagga ggtggaactc acagcaagca gaaaattagt |
| 39961 | tgaatggcac cagctttagg aactgccagg atgcactttg gatcatgaat aagtaaagaa |
| 40021 | aagaggtcat cacaggttcc agaagagtga ggacagagag aagcaacaaa gtgagaaRag |
| 40081 | gaagaaggaa ggagacaaag aagacaggat acagagaaga agggggctgg aaggagtggg |
| 40141 | tgaaagaata agaactcaga aaagagattt tggttcttgc ttattcagga gcttaaccag |
| 40201 | aattctgtct aaactcttta aaagatgcag aaactcaatt gtctcactca tccctgggtN |
| 40261 | ggggggggga ttaatgtatt agtactattg ctacaacttt atgaggcgct tcctatgtgc |
| 40321 | taggcacttt gaacacactt ccttatttac ctcttaaaac aatctgtgac tcaggtatta |
| 40381 | ttgtctccat tttatagaca aagaagctga agctcagaac agctaagtaa tttactcaag |
| 40441 | acaacccagc caactgctgg cagagacagg atgaaaacac gtatcttatt ccaaccaaca |
| 40501 | tccatgcaag taRccactac accgctcggc cacaggaaac aagcactggg aaagaccctt |
| 40561 | cccctgacac aggaggccga gcggctgatg agcaaactcc agagagcact gggctctgac |
| 40621 | catccggtgg cctcccctgc cagagggctg gggcaataga cacatcccca cctgtgctaa |
| 40681 | agtcctgctt tcctctccac aaccctcccg ccccatccac actccacact gcccttgagg |
| 40741 | gaaaaatgag gacccagagg atggcctggg gatgaggggg gactgctcat tagctaccca |
| 40801 | gccgtggtag ctaatctaag atccgggaat aaaggccctc cctgaatgac cccagggcaa |
| 40861 | aaccaggtca aggccacttc caaaccccRc cccgacagag ttaaaggcga ggatctagaa |
| 40921 | aggctgaaaa cacaaagaag ttaatcagct ccctgaaatg aaaggggaga aaagagactg |
| 40981 | gataaaggga tcagctgaag gaagcagcct ggcttgggag agaacactcc actcagccac |
| 41041 | gagtccacag actcaggcca gcactgcccc gggggccagg agatcttggc Ratggctctc |
| 41101 | tgctgtctgt ttccttctct gttagacaag ctctagggtc tttgcacctg acagctgagg |
| 41161 | atagggcagc acctgtggcc aacacagcct catctggagt gggtccagcc aatgctgtga |
| 41221 | ccagcggctc tctgtaggtg cacaccccat aggtaccctg gatccacaca gatgccccct |
| 41281 | ctaattccat gctgcctctc catctgcctc ttcaaggtaa aaggtggatg gagctagggg |
| 41341 | gtcagaagga gtcacaggga tgggtgagct aagaggacaa aggaggcgtc tccctccaag |
| 41401 | acaggatcag cgtgtgtggg tgggaggtat acaaaacgcc atctctcctc cacagacacc |
| 41461 | cgggaacact gcctgaaact tccaccactg atgtcgctcc agtggcttaa aaaccaccct |
| 41521 | tccaaaaatc cccagttaaa atgcaaacaa ctttcttaaa gaggatagca ctatcatggg |
| 41581 | ttcctcactt gatcaactca gacactgtca gaaccagagt gaatcttgga gaccaccaag |
| 41641 | tccaacccct tctttagaca cagaaggaaa ccaagcctct aagaggtaaa gtgacttgct |
| 41701 | cagggtacag gaagaggtgg cgcagagcta gaactgtaac caggtctcct aagtctttct |
| 41761 | gcagcatggc cctggcccct gagggaggtg gcccaggatg ggggcgggca gggggtctgg |
| 41821 | aatcttggca tcgtgctccg gggctgctcc ttcttcctgt tcagccacca cRgcaaacaa |
| 41881 | gagcccctgg ggcctcagcc agcgactgac ccctccctga ccctgccttc cggaaggatg |
| 41941 | aatgcacatc agaagccagg gccaaaatat tcctgggcac tgtctgctgt gcacccagct |
| 42001 | ctctttttcc cagcctctta ggagggagaa gtttccaaaa gggacagctc caaaggcttt |
| 42061 | cacagaaggg gagcctctct aagaagtgct cctgtgtttg ggcaatggag gYaagtcagg |
| 42121 | aagacagggg gatggaagaa gaaagtaatt ttttgaatgt cacattttca ccaaaatggc |
| 42181 | atctgggaac tttcaagatc atctgactaa taccaggttg tattcgttat ttgcaaagat |
| 42241 | ggaggtggaa tctcctttga agtagtacaa ccaaatgatg tctgtagcct tMaaatgggg |
| 42301 | taacaagagg caaagaaatc tggggagagg aaacgtgtcc tgccaggggc tgagggtctt |
| 42361 | ttttgctctc ctYctgacag tctcacagcc agccacgtcg tggtagggtc ccacagagct |
| 42421 | gcccagcatg gggcaggctg gccagagcag ctggctcttt gcaaccaccc agattgcctt |
| 42481 | tgctgggaag ctcaggctca tttgcacact ggcataactc cacgtcactg accagcccat |
| 42541 | gggcaacagg actcacagcc aggatgggtt ctccctgggg ctggcacgac tctgtcactg |
| 42601 | cacatgtgca ttttcctgat cctgggcaag ttactgaaac tttctcaacc ccagttctcc |
| 42661 | agctataaaa taggaacacc ccctacatca caggattgtc acataaggga aaatgaggct |
| 42721 | agagaacctg gggtaacgtg gaattgactc tcagtgagcc tggttccctt tctctcgcaa |
| 42781 | taaaagccgg caccacctgc tccaaaggta aagcctatgg ggaaagtctt tcccagtgag |
| 42841 | ccctgaacaa gacagagggc ccttccccac aaagcttccc aggcttggag aggcgggtgg |
| 42901 | tccccatgac tcattggtgg tggtgggctg actcatttgg ccaactgtcc ttaatttccc |
| 42961 | tcctcccatt ttgggagtcc tggagcattc gttccccaaa tgaggcttgc agccagaatg |
| 43021 | agctgtgatt agcacagcag agaccagaag gatggtgctg gggggaggcg gggtaagctg |
| 43081 | cagagagttt cctgaaatta aggcaggaga agcagggagg caaggtcttt ctccctgggc |
| 43141 | catttagcag acagagactt cctttagagc caaccttctc aacctctgct gggcaaaatc |
| 43201 | aactcagagg actgcctgca atagccctaa tatggccctc agaggcagac ctggggtgtt |
| 43261 | tatcacagag cacaccatga aatttcaaac cactatgtag gtaacatagg tgggtgcata |
| 43321 | aaacagaaca ttgccccatt gcactgaatg cacaatgaac agtgggcccc agaatgcggg |
| 43381 | ctgtaccaga tctacataaa tattacaatc tgaaggcaga tgaacaagag ggagtttgca |
| 43441 | agggttgggt gagaccacgc tgtacaaggc ccaggtgctg tatagatgat gcagaggtgg |
| 43501 | gtagggtggg aagagaggtc aatgtgaagg ctcacatcca attgtttttc ctttcctcag |
| 43561 | gaggaggatg gatattcttc tgttgtgggt ttcaattcca tgctgtcaaa taacctcttt |
| 43621 | agtccaatgc tctgccactt tcagccaggc ataagcaaga caattgtcac tgaacattat |
| 43681 | tgggagtaac atgcaatggc acatgcacta atgcgaacat cagaaggaag acccggagtc |
| 43741 | tagtctagcc ctatcacttc tgcacagctt tggcctgagt cacttaaccc ttccgagtgc |
| 43801 | tggtctactc atctgctaag tgggacaatc acttctgaca aggctgctag gaagattaca |
| 43861 | ttgagagtgc actggtgatt attcagcatg tcagggaggg cctaccactc cgggcctcca |
| 43921 | cggagtggat ggctaagatc ccctgactcc cagtgcacac tcctcacaca tggctgcacc |
| 43981 | tctttcggcc tcaggggcca gcgaggagcc taccgacaag gtgaggtgaa gagcgaagag |
| 44041 | aatcagtgat ggaaatggaa gccagcagtc agggcttgca agctcagccc ttctcaggtc |
| 44101 | ctccaagaag taaatcagct gagggtcaga ccccaaggag cttcacggcc agcaactcag |
| 44161 | cctggtttcc tctcttttcc tcgcatcaca ttcagtgacc tatcagtcta gatagagggt |
| 44221 | gctttttcaa ataaaccctt ttccaacttg tctaggagaa caatcataga ttttgatccc |
| 44281 | aaagttaaat tggcctgtca gatacaaagc tttctggagc cagccagcat ctcaggatat |
| 44341 | cccacactcc tcccaaatgc attaccaccc agcaggtagt gtcatcgcag tttctctggt |
| 44401 | ttcagaataa taactggtgg gaaatgccca tggaattgac actcagcact ttgaagattt |
| 44461 | tatcagttgg gtaaatatca cccagaagac aggagaaact ggggttctgt tacgacatct |
| 44521 | acaactagaa ggagtgctga tcacttcttt tccccataag gtctaggatc ctataaccac |
| 44581 | gacaggatca gaggcaacct ggtggtgaaa ggtagacaaa cccaaactcc ttacatgccc |
| 44641 | agttcagctg tcctcacaat catcctacac atcacatgca tcctgctcaa gagctcgtgg |
| 44701 | ccagcctcct ccttcctaca gtgccctaat gccctagtga atcaagcccc accacagtgt |
| 44761 | ggacgctcgt aacatacctg tgactccccc cagcagcctg ggatcttcct tccactccat |
| 44821 | tcacgctcat ctctgctcgg gctccaagcc tcctgggccc attcacacct ctgagcYttt |
| 44881 | gctcaggtat ttcccactct tgctctcagt ggtctgcctt ctatttgtct accccaactc |
| 44941 | tatgcctctt tctatacttc ccccgctact ccaacctgca ctggtccctt tcctgagcct |
| 45001 | aactggcact cacattttgc accaagttgg cacttaattt taaatggtct tgtcttgaat |
| 45061 | aatatgctaa ccacctcttt cacctcctcc agaacagaaa aggtgcctta cacagtggag |
| 45121 | aagSctttag agccagaaaa atccttgatt ggaaaaatca gactttacca gcaatcacca |
| 45181 | ctgcctttgt caactcaatc tctctgagcc tcagtttcca tctctgtaaa atacgaattg |
| 45241 | taatcttaat ctcaaagggc tgttatatgg ctttaacaag ttaatatcta taaaaagcct |
| 45301 | agactcagta actaatgcat agaaagtaac ccccaaaagg ttattccagt tcccctttgt |
| 45361 | ttcttttatg tcttgcagaa cctagcacat tatggcaaca caagacattt ggtatatgtt |
| 45421 | tgattcattt ataagtagta taaagaaaac cagtaactgg agggtcatta tacaaagtag |
| 45481 | cagcatgcgg tcaaaacaca tggatctatt tttcaattac ggacaaaaag ttgagatcta |
| 45541 | tggagggtaa aacagtctac tttaactaaa gaagttttca ggattacaaa ctgccagtct |
| 45601 | ctctgtactc atcactcagc caagcagaag tttcagcaca aatgttcatg ctggcaattc |
| 45661 | atactacatt ctttcttaca taagttgtct ccatccccaa actccataat atgtatggtc |
| 45721 | cacagaaagt tcacttcacg tgctaaatgt ggtacaatcc ttgctacttc tttaaatcaa |
| 45781 | acacagagca aaatttttct ggagttcttt ttaaaattat ttagaaagaa Ygccgatcac |
| 45841 | ttcttttctg aagcagaatt tttgtcattt atctatatat gtaacatata taaatataat |
| 45901 | tgtatatata attaaattcg tcttctaata agacctttcc tccccagatt ggagacaaca |
| 45961 | tagtagacaa ccatgaatat tagtgaggtc attcaaccaa aatcatgctg agaataatat |
| 46021 | ccaagtcctR tgagcaatta tgaaatccat gcaccaaaga tgatgttgga tgtggtggtg |
| 46081 | tccagtgaga tgaaaactgg agtggaattc agatggagtt tcagcagaac cattttcacc |
| 46141 | tggtggcctt ttggccttac tgttcttaca gtttcttctc actgtctctt tttcacatac |
| 46201 | ttggcatttc tctggaggaa aatgtgtctt gccactgcat tcacttttca gctcgtggct |
| 46261 | catgctaccR aaacaatgga agtcaacaaa aggcttgaga cattgcttYa gtgtgaatat |
| 46321 | aaacaagcct agaaatgtaa ttttagttag tgatcagaat acatatccgt gaaactaatc |
| 46381 | ttctccacgt ctccccactt cctggtggtg agttaggcac caactcttcc aggtcatcta |
| 46441 | gctataatgt aaaacattac agaactaaat ggggacccca agaggtcatc taacccatgc |
| 46501 | ccccaactct atgcaagaga aatgcttggc tatcagtgag ttccScatca gcctcgacag |
| 46561 | tctaaaatgc ttggtgtcaa aagaactgct tgtctatttg ttttaggcac caaggtgttt |
| 46621 | ctcctgtgct ccctggagac caaatgtaac tggtcagtgc ctttatctca gtgatacaca |
| 46681 | taacaacagc tattaagtga cccctaattc ttttccccaa gttaaacaac cccaaatcca |
| 46741 | caaacctttc cttaaagggc ttattgttca tcctgttaaa catattgtta ataagctttt |
| 46801 | ctgtactttt tggatttccc acagtctttt tccgctaatt caaacacagt ggtgtcaaat |
| 46861 | attaaataga gctgaagcat tgcttcccag accttccaca tcattcctag tttaacgcag |
| 46921 | cttggttatc atgctttctt aaaaataaac aacacaatac tcctccagct tgtgatccac |
| 46981 | tacgacttcc agatctatct cagctataat tgtgccttgc caggtgatgg ccaataatac |
| 47041 | aatcaacata atccagtcaa gcagattgat gctaaaccat tgatgaccac tgtcatcacc |
| 47101 | tctcaaaaaa gtatttccta gtctggtgta aagaaagatc aggtaaccaa acgatgtgtg |
| 47161 | tYgttcacac aatgtttaat aaaatacaaa gcatagcatt ttctaacttt tagtttgaag |
| 47221 | actctttctc ctcctggtct atgcctgaaa actaacttct gggtgaYttc atatgtgatc |
| 47281 | acagggtaga acttggagaa aatgaaacag atatttaacc tgcttatccc aaacactgcc |
| 47341 | ccttttcagg aaacttggag gtaactgggg taacttagca ataaaccccg ctgaaagtta |
| 47401 | tcctgaggtc tgaaagtcac tgatcctctc agcacaatgg agtatccact aacaatggct |
| 47461 | aaagagcatc tcttcaataa acttagcttg actactacca cctatcatga ctagctcatc |
| 47521 | gaccaccctt caggatcatt tcatgtgcaa ctagtaagaa ggtttcaatt cagtagtagg |
| 47581 | tctggcatca gtaatcttct ccaccatcca aggaagaaca tgttatctta ctggttgttt |
| 47641 | tgggaggtga agtaagaagt tgggaaagtg tgattctgaa tatgaagaat ctgagtactt |
| 47701 | aggaagaaat atatctcagc aacatcctca acattccata attaaccact ttaaggagcc |
| 47761 | acagccatta aagaatctgt gctgacccca aagagaccat ttctctaatg gcaacaactc |
| 47821 | cacatctccc aagttgggcc ccactcattg tctcctatca tttgtctcat acagatgaaa |
| 47881 | ctctaccatc tatgttcact taactcataa gacccaagtt gctagagatg aataatatta |
| 47941 | gtcatgatgt ccacaatgga gagtcgctgt tatgcattgc tggattattc atagacactt |
| 48001 | ctggtaaaca tggcactact gcagtttgga ctctgtacca aaactagaaa aactagtaaa |
| 48061 | cagctttcca aaatcttgtg gtgagatttt tctggctgtg aaacatttgt tgcatttggc |
| 48121 | catgaaatag tttacagtac tgatcatgaa atgcatcagt agcctttcac ctggtaatgt |
| 48181 | gaaattctct ttccatagtc ctgaagctta gaaaatgccc tattaccacc accaccacca |
| 48241 | cccccaactg agcagtgcaa tactctcatt tctaaattag ggatcagcaa actatgtctt |
| 48301 | gtgggccaaa tctggccaac tgcttatttt tgtaaataaa gttttattgg aacacagcca |
| 48361 | tgcccattcc tgtacatatc ctctacagct gttttcatgc ttcacccaga gctgagtagt |
| 48421 | tgtgacagat gtttgcctta caaagcctta aatatttact atctggtgtg ttacaggaga |
| 48481 | aatttgctga tccccattat aaatgataag gcaaaaaaat ttcttattca gaataacctc |
| 48541 | ctctccaacc caacaaaaga aaacaaaacc agaaaacaac catgacccca gcgagtagtg |
| 48601 | ttcgcatact taaaagccag cctaactctc ttacaaagta tactctgaaa taagatgcat |
| 48661 | acaaaacata aaatactatt tgatggcaca gtagggtgac tgtagtcagt ggtagcttaa |
| 48721 | ctgaatattt taaaataatg taaagagtgt aatcagattg ttcataactc aagggataaa |
| 48781 | tgcttaaggg gccggatacc ccattcttta tgagtgctta tgttcacatt gcatgcctgt |
| 48841 | atcgaaacat ctcatgtacc ccataaatat ctacatctac tatgtatcca caaaaaattt |
| 48901 | aaaaaatttg taaaagtcta tatataaaat acttacaagg ttaatttaaa ttaaaattcc |
| 48961 | ggaaggattt ttttaatgaa aggaagttct gaaactgatg atttagggat ataataaaac |
| 49021 | tcaaatatgg ttctagttac atggagtgat ttgggtttct tagaaaagaa gggtaaRagg |
| 49081 | aatgtgatgt aatagacaac tggtcatcaa taagaataaa ctgaattttt taaaaaatct |
| 49141 | ctaaactgta caactctacc cagagttcta aagagaaggt cttccaggta catgcagagg |
| 49201 | aaaaagaaat ggatagggga atgttttctg gtggagccat atcttgaatc atccctaaca |
| 49261 | tgtgctgaat aatgacctag ctccatccag agaccaaccc agggacccat tcccaccaca |
| 49321 | taacaatcct aagtcaggat actgctagag gcatccatag attaaggcaa agatccRctg |
| 49381 | ttatgagaat ctgaacaagg catgccaacc tcaacttcca ccttacccag actggcagcc |
| 49441 | cttgggaaaa gctgccccaa agccatctaa gtgtgcccag taggcacatg gtcatgttca |
| 49501 | gttagggcac tcaaagctga tcttcagttt tcatttttca tttatcacca acactgaaaa |
| 49561 | atggggagat ctcatcaaaa tgtttgaatt gtgacctccc ttgaaaaaaa aaaagatctg |
| 49621 | ggaaccctgg gcccacctcc tgcaggctgc atcccctgag ctgagtcacg gctgccccat |
| 49681 | tggaagcaca ggtgcttcct ggtttgagac actccctacc actcctgttg tctccctgtg |
| 49741 | acctgagact gagcctcaag ggctgttcca catctctctg gcattgtcat ttatgtcaca |
| 49801 | cctgccctgc ttcattcacc aaagatacct gctctgtcag catcggattg gcaaccccag |
| 49861 | agctaaccat ctatggtaac tacatgtttc cgagtggatg aggaacatga tttctttctc |
| 49921 | tctttttcat tctctggccc atgtgtgtac ttttgccctc cctcttggtc agtgaccatc |
| 49981 | tatagacaga aagcacatca ttcttggcct acaacccatt atcagacacg tgaaaaagtc |
| 50041 | agtccctgtc ctcatggccc actattatgg acatttgatc atcctggatc tcctctatcc |
| 50101 | ttagttattt acatggatag tacacSttca tgccagacct ctcatccctg tgagcagccc |
| 50161 | aaagatagta tatgcaaaga aagcccgagt catgaggaaa gacacactct aggtagaaag |
| 50221 | cactggccca tcagaatcaa ggtcctcttt tctagagagt aaattagagg gtgcaatcgc |
| 50281 | ttcccatgga agaagatatt ttattcctta ggtgacttct taattgaacc taatgtctca |
| 50341 | aattttgagt cactatagga agtgggtgat gtgtaataaa tatgKgaaac ttcattttac |
| 50401 | tcWgtgttca tcaaaagata cRtggataag ctaaatgcgg tttacacatg caatgaaata |
| 50461 | ctatttagcc ttcaaaagta gggaaagttt gacacatcta cgtaaataat ccttgaagat |
| 50521 | attacactaa atgaaataag tccgtgacaa aaggacaaat gctgtatgat tccatttata |
| 50581 | caaggtactt agaggagtca aattcagagg gacagaaagt agaatggtgg ttgccaggag |
| 50641 | ctggggaggg aatggggagt acgtgtttaa atggtacaga gtttcagttg gagaagatga |
| 50701 | aaaaatccta gagatggatg gtgatgatgg ttgcacgaca atgtaaatgt acttaatgac |
| 50761 | acagaactat atcttaaaag cggttagaaa ggcaaatttt atgtaatgca tatttcatca |
| 50821 | caataaaaag gaaaaaaaaa accctaagga gggcaccata ctggtgtggg gctttctttt |
| 50881 | tactgctagg aaWacttaca aagtccataa tgcaaggcat aacgtgatat tcagtctaaa |
| 50941 | gaaagatgga gtacactagc agcagcatta cagtgtcatg ttcatctcca cttggcccca |
| 51001 | ctgggggcca gcaatgctca aaggtcaaga cctggcctga catgctcacc tggttctgtc |
| 51061 | tgacctagag gacccctcag ataagagggc ccatcttcac acacaaggaa agcctacccc |
| 51121 | gggggttcca gctgacagct gcaccaaccg gcagcttggg aggctacacc aacagcacct |
| 51181 | tccaggcaat gaaaatttta acctcccacc tattctaagc acacattaaa tcttgcattt |
| 51241 | ttgtgcaaaa cttctattct tggcacaggt gtgacagcaa ccttggacag gtatactaag |
| 51301 | tcggttcaca taatcaggct ttcaatccat caattttggc catggagagg gtgatgatgg |
| 51361 | tgatggtgat ggtgatgggg gagtgggtga cagcaaccct ggctcagaga atgaacgctg |
| 51421 | gcaccaaccc tgggcattgt tcactttcca ggccaccatc tgtcagccaa gccagtggca |
| 51481 | actgctgttc tagaccatgc ttgtaaataa aacaacaagg ttcaacaaat cttggcatgg |
| 51541 | aagtgggaag aatcatctca ggagtccctg ccgcatgacg gcgtgaaagt gaactcacac |
| 51601 | aggatgagct tggttggtca agcaaatttt acagagatgc tataggagca ttggaaacag |
| 51661 | actttttatt taacagaaac ttacacacac ttggtacaaa ccctacatta tgcaaacctt |
| 51721 | tatcacagtt ctacacaggt tgtcacttgt gggagttggt tggaaagcag attattatga |
| 51781 | taattattag cattttgaca agggacattt tctaccagaa aacgtcagag aaaagggcta |
| 51841 | caaaatgata cagacactct cggtgatccc ctccaccaac aggactggct ttgaacagga |
| 51901 | aacctgaaag gactggcagg aagtcggtgc ccacctgccc tgccctttca gaggccaaag |
| 51961 | agattctcag gctcagaacc atttcagtaa cactgtgact caaggacctc tgctgaccag |
| 52021 | aatacccagc Sttagactat tcgtgtgtta ttatgactct gcttacaagg gagggaaacc |
| 52081 | caagagcatg gctgtgtgaa agcccagtgc agtggggagc ctgcgggtag aacccagtgg |
| 52141 | ctcagccagt cagccctgca cctttgctgg gccacagaca cagcgccgag atacatccca |
| 52201 | tcggccaaaa cgcccacttc cgccagctca gccagaaagg gccaYgttag aacacccgcc |
| 52261 | gtcttccttg gctcccacca gcaactgaat ttgaggttct catccttcag gtcggccttc |
| 52321 | ctagcccttg gggacagctc tggaagctca ggccccagaa gccagctcag cagaaggttc |
| 52381 | cctttcctca tctcttctag gagttgttag gtgactgaac taatatcacc tcagctgatg |
| 52441 | cttttatttt tgcttttttt tttttttttt ttttgctgca ttattttaaa caaagctctc |
| 52501 | tgtgttcgaa tctattgaac ttgaggggat atattccatc aatagatgga attccttcct |
| 52561 | gagctagacc tcatcagaca tgataactag gagNcaggct gcttctgaca ttcagataaa |
| 52621 | aggggcttgc ctggggtgag ggggcaatga gggtggggga aactgctgat ttgtggctaa |
| 52681 | gttatttcag tcaaatctta acacacaatc atttaacatc accaacactt tcaatagact |
| 52741 | tttgaaggag gaaaaaaaaa ctgagcactc ctcacttacc acatccaact cgcagaaata |
| 52801 | aaaataccta aagcacatac atgaatggct ttttaaaaag cctgagagag cagagttgat |
| 52861 | ctttattttc ttaYttaaaa tgtggggcat ttttctttag agttaatatg cagtctttta |
| 52921 | ttttacatta agtactctta tgatttcatg tcttcacaaa attgcgctta acttttgaag |
| 52981 | taggaggtac tttaaacaaa aatactgtgc Matgtcagca cttttttcct tttaaatatt |
| 53041 | gaaataaata aaatagttaa ctcttttttt ccatcagttt gtttgtgttc agttatttct |
| 53101 | actgtagagt aattccaggc ggacctgggt actttctcac agacaatcaa agaaaatata |
| 53161 | atgaagattt aaaaagaaaa aaaaaatctt cttattgcac aaactgaggc cattcccaag |
| 53221 | gcttctgtgg gctgagctga gttgcgctgc aggattttcc actcctgtgc tggctcctgt |
| 53281 | gttgtctgaa acacacgcac aggagggggc tgggcaaggg aagaggaaga agaggaggag |
| 53341 | gaggaggagg tgccaatcat cctggcttct tttcaccttc ttcggaaatg gcttgcttga |
| 53401 | aggatgctgg agtccccagg tcataatgtt gactgcagag ggaaaggcaa tgaaagataa |
| 53461 | aacaaaaaac aacagcatgg aacaactact taaaaaagaa agatgaaaat taaggggtga |
| 53521 | cagaaatgaa atgaattaaa aaaaaaaaag aagaagaaag aaagaaaacc accaactaat |
| 53581 | tgccaagtta atgaggctag gccaggtgca acaaaaatca gggtagtgtg tccagcgcct |
| 53641 | tgaacttcag cagcaWgaga tSagccagga atagggcagg tcagttgcta ctgggccaga |
| 53701 | ggcagagaga Rcacagctgg atggtcaatt cccaggtggt gtagcaggca gaactgggtg |
| 53761 | ttagcaaaaa aaaaaaaaaa aaattcccct ccacttaRaa accaaacaaa ctgaatcttt |
| 53821 | tcacagttaa aacaccccaR aaaggacctt tatgatgtca tctctctccc tgYcatccaa |
| 53881 | gtaRaaatga gtagttctat ggtatagaac caattcagcc acagaaaacc ccatcaRatg |
| 53941 | cctcatttgg cctaagccca aaaggacact gcttccttca caagcactca cactcagccc |
| 54001 | caaccctgcc ccaagtgatt caagtgtgtc aRagttactc ggagacagtc cccaaccaag |
| 54061 | agaccagttc cagtcctcta ctagcacagt gatccctttg tcagccttgg aacctggtgg |
| 54121 | tggttcacac aggtgggccc ttttgcctgg atagccacgc atagcttttg cttctagctg |
| 54181 | aaatgaaatc tgccattttg gtctttggaa cggagtagtt gacacaaagg agtgagcaat |
| 54241 | gaaaccaagt actgccacat ttcaccccgg aagcagtttc tgaaagcaga gatgggtgtc |
| 54301 | ccaacagtgc ctagatcaca ttaagctgag cgataaggga gggagaagag gSagaatggg |
| 54361 | cctaaatgga aaatggccaa gccagcaagg aaaaaagctc aaaagcaaat gagcagctca |
| 54421 | gcctcttcca agttgaaagg cccctcccct gccccacctc tcctggtacc caagcatcac |
| 54481 | tcaatggcca ggtgcacagg cctggattag atgctcttcc aaagctgaga ctccattcat |
| 54541 | ccaactttcc tccttgacca agtcctccct ccacacctgc tcctgttcct tgcctctacc |
| 54601 | ccatcccacc ccactgtctc cctttctgat ccgctttgct ggtgcaccct acccccactt |
| 54661 | ccctcatgct ctttgctgtc accacctgcc tctagctcct tcccttgact gatcaaaccc |
| 54721 | aggtgacctt tcctgggacc tcccacagaa cgcctgccga ttgttctctc catgggcatc |
| 54781 | tgtcatcatt tcactccctg ccaggagtct ttctgtccct ctcccagtgc cctgacccct |
| 54841 | ctggtcagat gttacctgcc actctgtagt gagtctgtct cataagctta ccccacacct |
| 54901 | ttcagtttct tcaaatttcc accccgacct cccaacacac acacacatgt atttttcctt |
| 54961 | tgccccacta aagccaccta gaaaatcatg cgtgagtccc aacttggcac tcctaacttc |
| 55021 | cattttctct tttgtttcct ctggtcaggt caactgcagg ccacaaaaat gaaacaatgt |
| 55081 | gcagctgtgt tcttagtctc ctgctctcaa tcccctgcaa gaaggcatag ttttctaccc |
| 55141 | ctccacacac atggtcaacc cctccccacc cctgtcctga catccccctt ttctctcctt |
| 55201 | tacccctcac caccacctca ccacctccca cctgccccct ctgacttaac cactggatgc |
| 55261 | tgtgtccaga gacttctcca ttctttctgt caacaatatt tattaagcat cctctatgca |
| 55321 | tagagtgaga tttggtaaga cttccactaa ttatctcaga aactgctcag ttctcagttt |
| 55381 | atttcaacaa ataattatgg cacactggtg tcaggcactg tctggaggaY atgagtgata |
| 55441 | tcacctttgc cctcaaggaa cacgccatct gtttgagaag gactacaagc aaacatgatg |
| 55501 | tgttgtgata agcacaggag cggcaggaac aaaatgctat ggaatggtac ttaggcgggt |
| 55561 | tttcaaatgg tgatttttgc cattttacat gggttaggtt gtgattccca gctttgcttt |
| 55621 | ttgttcgaat attatctcca ttgtctctga caatagacat tcttgcctac agattcaaca |
| 55681 | gcaacggtcc atctggagct ctttgttgat ctagatggct tcctccaaga aaatcttctt |
| 55741 | gtctactgag atccatggca aaaataatca catactaccc catgacagct cttacatgat |
| 55801 | tttaactatt tgtgaatttc tagtctgatc cactaggtca caaaataaat ccaatggcca |
| 55861 | ataaattcat tgccccatgt ttaattttat cctatccttg acagtgctga gcagtcacaY |
| 55921 | gcatactgtt attactcagt ccctagttgc caaatgaacc gaggtatatc tgaatgaaaa |
| 55981 | tgttatcatc caaggtctgt aaacagaata gcctgaaatc tcagctgaaa ggccaaatgt |
| 56041 | ctgagtcacc agtaatgggg taaatcaatc acatacctcc Kgatgtaatg cactgagaag |
| 56101 | gatacaacat catttctaag gtattctaac caaaatgcac aacttcagtt tagtcattag |
| 56161 | aaaaacagca tccaagcctg aactgaatgg cttttgctat gaaatgacta accagtatcg |
| 56221 | aggttgWgaa aaacaagatt gtagtactaa ctattccaga ttaaaggact tgtaagagga |
| 56281 | catggcaacc tgtgagccca gactggatcc tggaccagaa aaaggacact agtgagacaa |
| 56341 | ctggcagaat ttgcataagg tctgtaggtt atagcattgc atcgatgtga atttcctgat |
| 56401 | tgtgatcatt gtactgtggt tatttgaaga atctgggtga aggacatatg ggaattcttg |
| 56461 | gtaaaatctt tgtaagtttt ttgtaagtgt aaattatttc agaagtaaat tttttaaaaa |
| 56521 | gccatagtta ttggttgagt tccagtggtg agatactttg aaaacctaat tgccaatgtt |
| 56581 | ttttatctaa taattcagtt ctgacaggaa tcttccatca ttcccattta ttttgtaaac |
| 56641 | caatcatgca gaatggcttt tcctatattg ctattttgaa taggtgatca gaaatcactt |
| 56701 | ttattaaaca ccacaatatt atataattca atgtttttgt gttagaagca attgggtttg |
| 56761 | tgaccagggc ccaaaatgtt cactccctgc caccattact cccgctacca ctggcaccac |
| 56821 | aaaaacagtg accatttagg aagctcaact ttagttctcc cagaaagttc atgaggactt |
| 56881 | agacattgct acttctcaga aagattctaa tgtgtcatag tttcatttgg ggtttctgtt |
| 56941 | gtttcagttt tttcttttac agaatcaaat acctcctttt catccaataa attcttgaat |
| 57001 | aaaagaccta caggcatttt actgatttca ctcaagaaat cttccaaaga tgatctttgg |
| 57061 | ggaagtcagt atagacttag gaaagattat tagaagatcc atacatcatt tactgagcct |
| 57121 | ggaactttag acaagtcatc aaacaggatc aaatctcagt ttcctcatca gtaaaacggg |
| 57181 | aataaataac atctacccta cctcatagag ttaagcatca tcatgaaata atataagcta |
| 57241 | catattctaa tagggaaaga aatgtccatt tcctctctca gaaactgagg cagagatccc |
| 57301 | tgtcaacaaa ttcacacagg gagctgagcc cagagcccag aacttgtcaW ctccactcta |
| 57361 | ggcttccaca caccagatca aagacacctc actcagaaaa tacacaacac aagtcacttc |
| 57421 | tcctccaaag gctgagcaaa atcccctgag gaaaaattcc taacagatga gtctgctcca |
| 57481 | attttaagta caYgttcaag atcatattca tcagatgtaa caggaggtat actgggataa |
| 57541 | aattaattaa ttttattcca ccatgaggtg agaaccccca aacacccttt agttctctcc |
| 57601 | ctgagtaatg atcttgcatt ccctccagtg ctgtataaag cagtatcagt ctgtagccca |
| 57661 | ttagatgcag agagagatgc aSagtaagca ggaaccctta aagggatgct attgatgtga |
| 57721 | acatcacatt tttttttatt aaattcttct ctctaaaata ccggtagcaa attggattac |
| 57781 | tgtctttata gtacctctaa aaaattaaat gcatatttct aattcccagg gtcatagtga |
| 57841 | ttatagtgtc tgatctttac atagtctctc agtaacagaa ataaaggatt tgattgttct |
| 57901 | aggtagggaa aaaaatcagc tagaccataa caattgtcac ctttaggtca acagcccccc |
| 57961 | tccccagggg attaaaatgt tgtaacaaat ttagaaagtt ctatatcttt ggttcccctc |
| 58021 | tctttggcct gggactcaga aaactcaggg tggtacttta ctgagctgag ggcaaaggaa |
| 58081 | gctggccaga tYgttttcat cttgatgact ctaatttctt ttgtatttta aattttgagt |
| 58141 | agtgatactt atttaacaaa agcaacaaca gaaaggtgtg ttgactgttc acacagctcc |
| 58201 | tacaattcct aaggtcacaa aaaaggtgat ctctttgtac attgtctaga agtgatgatt |
| 58261 | aagaaggtaa gttatctaca atattccgca gagaatctga attagaactc tcttttacag |
| 58321 | gatctaattg cttactcttc cgttgttgaa atttctttta atgacgttac cttttaaaag |
| 58381 | ctatattcat tacaaaaacc cttttcttca gttggaattg agaaacagca aagtatgtat |
| 58441 | acatgcatgc atatatattc cttaattata gactatgcaa ggggcaatgc aggcaacgat |
| 58501 | agagtatagg ctctacaatc aggttgccag ggatcaaaac ctggctcctt cctaggtgta |
| 58561 | catttcttaa ccttattact aacctcagtt tccgcaaact ggggattaat ataaacacct |
| 58621 | atctcatggg agtagtcatg aggattaagt gaaatattcc aaggaaagcg cccagaataa |
| 58681 | tgcctcacac aaaaatgtag atgactgcta tctagaaaaa gggagataaa Katatcacat |
| 58741 | ccaccactaa agtggttgga aaccaaggac agccagcttc ctcatccgac accttctctc |
| 58801 | tcggagcttc cttcctttgc cttaaWacat ttccctccaa tgcaatgccc atcccagtcc |
| 58861 | ctccaactta actcacttaa cctctctggg tcaacttttc tcaactgaaa agtgaaagat |
| 58921 | ttgaacttga tcatcttaaa agtcccttcc agtttttaag ggtataaatc ttgagtccgt |
| 58981 | tcaaagactc tataagcaag gtgagacaag atggtctggc catgctaaga aaccttacta |
| 59041 | tgcttctatt accccaagga agaaaagtga ggatatcaac tacctccaag aagagaaact |
| 59101 | gaagatcaaa tatcaagcta tactattaaa tataaaatag acaagcaata cccattctca |
| 59161 | gataaacatt ctcctgggag tttgccttgt tagttctaaa tggttagttt ctctgttaga |
| 59221 | ctccaagcta gactctgaaa ttaggtagcc ttttataatt tgctttaaat atgtggatta |
| 59281 | aaaaaaaaaa cacctcaagt gtaaaggcaa attgtcattg agaattctgg gactatgagc |
| 59341 | aactagacag aattcaaatg agagtgtaga ttggttgaac atatgtttat taacatagaa |
| 59401 | gctcacattt tatcaatgaa agcaagttgt aatcccatga atccaatatc tgagtcaaag |
| 59461 | gtacagagta aagaggtgga aaatgacagc aagtgtacat tgtatccccg tagcagaaac |
| 59521 | ctactggaac aaactctatt ggctgacgtg tgagcatgca gaatatgaca accttggagg |
| 59581 | tttatgccat atttcctagg aacaggccac atatagatta gacagaacaa cccaaaagtg |
| 59641 | gttgggattg ttactttcat tccctcaaaa ctgagaataa atgcccaggc gatcacatac |
| 59701 | acatctcaag catgagaaaa gtggaagccc tttctgtctc aggctaccag gaaagcacaa |
| 59761 | aggtctcatt tctgggtttc ttggcttgtc cttgtgtttt aggttggttt gttattttaa |
| 59821 | ggagcggtct aattcatgag gatctgcagg ggctggactg tcaaagtaga tcttcccaga |
| 59881 | gacactgata agcagtctgt taaccaggca gctacttggg tctcccaacc catgtcaaat |
| 59941 | ggttctgcct gtgtttccag aactgtgcac tcctggaggc aagagaccct ggccaacctt |
| 60001 | aaagggagtg cagaatcttc caaacagcca agttgagttt tgtgcctaat cagggatctt |
| 60061 | ctttaatttg gactcatccc cttggttttg aaattggtac cgagagtaag aaataccacc |
| 60121 | tagacatgca gtgccctgtg gccacaacct ccctattcct acccaaatcc agcagccaac |
| 60181 | tccaacttca gttccctgga tttcacttcc aggtttagcc aagggaggca gagttagcca |
| 60241 | agcacttccc cagccatttt ctcttgtagt tgaagccaga agcctgccat tctccacact |
| 60301 | gagcccctcc catagtccaa gcaagactca gatcacacac ctatgccagg gcccacatga |
| 60361 | gttcacatgc acacacaggc cacagagggc cagggggaga cactccagca gccactctta |
| 60421 | cactaagaag ggagtccaaa cccaggaaaa caaaaaagca agctggggga aaaaggaaaa |
| 60481 | agaaaaagaa aactaaaaaa tagatatgat ggttttgccc acattgcaag acaggttagc |
| 60541 | caagacatac cttttgacta ggacctcgca gccggtcatt cttgggactg tcagtgaggt |
| 60601 | gggtggcctt ctccatcgcc ccaggggcct tgagcagtgt tgctttggtt tatttcagga |
| 60661 | agtgctattg gaaacataaa acaaagtgcc tggcaggcac tataaaaaaa aatagaagaa |
| 60721 | atacggtagc cttgatcatc caatgggtat tgatacgttt cttaacaggg gcaaggggag |
| 60781 | gggaaacaca cgatgatgtc tccttctatc ttccagaagc acggcctcgg cctcgggtgg |
| 60841 | tggagtcact gctgagccca tgacgttctg cttatattcc atccctgcat ttggaagtcg |
| 60901 | ttctttgcca ggaggRaagt gaggaaaaac cagcaataac aaaacagcag ctctactgac |
| 60961 | ggaggaggag gagcccagga ggcggctggt cagggcccag gtgtggaggg aggccaggca |
| 61021 | taggcacccc gacttctctg gaactactga cattttctcg caagcagaga ggaagatgga |
| 61081 | aaggtcaggg aggagaatga gggaggggtc tgccgcgggg agccgcaaac tccgtggggc |
| 61141 | acagaatagt gcaaccgtct cccattgagg aaattctccc caccgggcgg cttgcctcta |
| 61201 | aacaggatat tgcttcgatt tctttgattt cccttctctc tctctctctc tctctctcgc |
| 61261 | aaaaaaagtc tgattctaat aacagctaga atatataaat aataatgttt taatgttatt |
| 61321 | ggttctttgt ttccaaccca aagttcctct tctcttcctt ttgccaataa atatgaaaat |
| 61381 | tgaggacatc aactctgctc ctgtcaaaaa gaccccctcc cctcagaaaa gagacaatcc |
| 61441 | gtctaacttc ctatttctat ggaaaataaa tagcgcatca cgatgccatc tctcggaaga |
| 61501 | acgctcattc tgctgttttg ttttgtttca gtagtttgtg cttctgttgt acatacccaa |
| 61561 | aacagagggg tggggggagg gatggaggga cacgtgggag gtgaagggaa aaaaagcaag |
| 61621 | gatagaggga aaatctaaag ttgcctttca ttaaaaaaaa tatatatcag aaccagcaca |
| 61681 | atgaccaaag tcagaggggg atggctaatt gcccagagag tctgttccgt gggtttagca |
| 61741 | actgcccttt ttatctgctg tgggcgaacc ccggccactg cggcgtcctc gacaggagcg |
| 61801 | aggctcttcc aaatcgtcct catcaaagcc gtggaaagtt gacgtgtcac tttctgacgt |
| 61861 | ggaaccgaat aagtcctccg tggtacgtgc tgcggccgct gcctccttct ccttcctgcc |
| 61921 | ttctcctccc aaatgagctg acatgtatga tgaatatatc agcacatggg ttaagcaaca |
| 61981 | gacagcatta ttaatattct cattacatta ttacaatgct attcttaata gcaagatata |
| 62041 | aaaccaYttg cacaccaaag ggattaaccc ggtgcatcaM ctggtgacgg ggatttggga |
| 62101 | caaagcaaac atttccaggg aagaggcttc ccccactccc cgaaaaggca tcatctctac |
| 62161 | catagtacat atgtccacag cttccgacag caaaagattc aaccatgatc tttcttctgt |
| 62221 | tgtgcataca cacaggcgct tgcacgctca ctcaaccctg ccccagacaa tgattttcat |
| 62281 | tggtggtttt taagcctggt tgggatttct ttggtgtttt ctgttggtgt ttctttgaac |
| 62341 | aatgtttgtc cttcactttg acagtcagct gtacaaaggt attactgtca gataacccat |
| 62401 | tcaaggtggt taagcctcca tctatttcat ttttatttta cccgcttcta atcccattct |
| 62461 | ttcagaaccc tttcaattct ccccttacgc catgtcgttc atttgcactg aaagcaaaag |
| 62521 | gaagttggaa agtctgataa gtggtaggtg ataaaatagt ttgtttcctt taaccacagg |
| 62581 | aggaaatttc tgtgatctgc atccacatac atgtgcacgt gtgcacacca cacacacaca |
| 62641 | cacacacaca cacacacaga cacaSacaca cacacacaca cacccagcca ccagccatgc |
| 62701 | ccaggtatcg gttactccag aaagttcagt tgtcaaatct cattcagggc actacaaagg |
| 62761 | tgagaaatag gagtagaaaa gccctaataa atcagagcat cctgaaatYt gtaaaactca |
| 62821 | ggtttaaact tttttgcatt ctaaatgagc cccaggactg ctcttgtata aaggaatgat |
| 62881 | acatccctca tggtgccaat ttcaggaacc tcactttctc ccaggaaaca gcagtggtag |
| 62941 | ggtagatggg aggggaagga agctcttcca agctccatac tcttctctgg aaggaccgta |
| 63001 | gggaggctcc agagaagtat tcgcccacct gccttaacaa gcacatctca tctcatgggc |
| 63061 | caatgacacc tgtttcttcc catggtatca actatttcag gaacattaga gtcagacctt |
| 63121 | gactaaatca tctaccaaaa ccaatccttg gcaaatgcag tgagaatacc taggctggta |
| 63181 | gaaaatggct ccctgagcca catagcagac agaaccttgg agggaaagct gagggaaggt |
| 63241 | tgtggaaaga ggctttgcat agaaaaaaac aacttcccac tcctgacatc ccatggcttc |
| 63301 | gattctagca cccctctttt gtaagaaagg aaggggagaa attacagcag tttgcaatga |
| 63361 | gaattctgaa gctgccccct ctaacccatc caaacctaca gcagtccata acgtgttgct |
| 63421 | tagaccatca cttcaagtga ccttgcttct ggaagtagag atttcatctg attgcttgct |
| 63481 | tttaaaaaaa cactgcattg aatttcatct gtttttttta agtatcttaa aaaaacagag |
| 63541 | tccgctatca caagggccct gtcttctacg ctagcatcca tagaataaaa aattcaatgt |
| 63601 | taggtaccat gtttggaaag taaatgaaYt gacagaattg cacaaatgga ccacccttga |
| 63661 | gagtcacacg ttgagggttg tgtgcagcag aaaccacagt ccttttaaaa ttactgattt |
| 63721 | gctactcaag ccagccttaa gccctaaaca gccctaccat caaaatataa aagggctaaa |
| 63781 | gctaaggttt gcagacagag caaggaaaga gctgagttgc tacagtcaac catgatgggg |
| 63841 | tcatctcagt tcttctctgg ggactgggag tgaaaaagac actggataga aaggtagaaa |
| 63901 | ggagaaatga ctcctcattg ctggaacaaa gcatccaacc aaactgtttc acctactcat |
| 63961 | atcaggggct gatgagacta aaaatggggt tgaaattccc tcctccctgc ctcacactgt |
| 64021 | ggtctagtgc tgacataata caatatgaaa aataggtagt accaaccaac catggccagc |
| 64081 | caaaaataat ggtttggtta gaactaaaaa acaaaacaac catttgaaca taacctgaag |
| 64141 | acctgaattc attttaagaa tgaaggaaca agaccaaaag tctctttctg ttattttagt |
| 64201 | ctttggatag gttaaaatat aaacatatac accaaatcaa ctgcagatta gccctaggaa |
| 64261 | tgcctgtcct tttgcctcat tgatctatcg gggtctcttc tgttctgttt tagaaaatga |
| 64321 | gattagtggc caatggcggt ggtgtttatg cagctagcca ggagcccggg gatctctgac |
| 64381 | tcccacaatt caagatgtgg ttgctctccg aatactcatg tttccacacc acaccacact |
| 64441 | cttcgggttt gccctaggca cttccacaga acaaactcat ctttctgtta catgtgcatc |
| 64501 | atccactaca agaaagcacg ttgccttctc tccaggtttt tccactctac ctcgctgtgg |
| 64561 | tccgtaaaaa cacgcacaaa gaaatatgca cctcatggaa aggccagaca caaacccaaa |
| 64621 | acttctcttt gccctttccc ccggatccta gggcataact tttggttctt gagacccaaa |
| 64681 | gaagaaaaat atcagaatga gctgaaaact cctctccact cgaaagcatt gccaccatcg |
| 64741 | cttccctctc cagtctccag caccacaaga tggaagagga atgcaagagg agcattcctg |
| 64801 | gtctacgggc acccggtgtg ggaaggggcc acactcacca gagcgtccac agtctgcgca |
| 64861 | ggacaccagc tcttcaggcc gcccactctt cttgttcatg ttggagcccc ccaagcagaa |
| 64921 | gtcacagtag ttattgggaa tgactgttcc atccggtcct ttctgggctg tgggaacatt |
| 64981 | taaaacattc caatttcagg cttacatgag tttcaactgc cccttcttct cctcctacag |
| 65041 | tgtggtagaa tctgctccag aaggaatctg aggttttatt ataaccaagt tggcaaattg |
| 65101 | ggctacgctt tcttttggat ccttccgggt ttaatggtac taataatatc ttgcaagtca |
| 65161 | cccagagttt aatttttaga gtattcccca gctctgtgtt gttgcagaaa tcaccctgct |
| 65221 | cctctgattg catccttaga tgatcaaacc atccagaaga ggtactggtc aagtgaacta |
| 65281 | ctctcagccc taaacaatca gccctgtggg cccaggcccc aggcatcaga tggaaaccac |
| 65341 | acaccatggt gtgaggacag ggagaggaaa gagccagtgc ctggaccttg ccattgggaa |
| 65401 | cagtgctctt gtttaggaga tattcttcct gYccctgaaa tggaaaacaa agaagcactg |
| 65461 | atgggaggtc atttccagga ataacaccaa agatgctctt ggggacttgg acaaacaact |
| 65521 | tggtggagta tcaatctccc tgaaggggag tctcatctcc ctgaagtcca gacccattga |
| 65581 | tcatggaaaS tgcccaagac cccaagtgcc taaaatcaga actagaccca ggcttctagg |
| 65641 | cacttggctc agcctcatgc cagagatgga ttaaacggtg agttactgag catgtcttta |
| 65701 | ggcttaagac cgtcacgaat aactgaacac actaacttca aaacccactc ctgctggagg |
| 65761 | tccttaaaca aaagcaaaga aaaatcttag gacatgactt tgaaaacaaa gcctgtgagg |
| 65821 | agaatagcac ctgtccttat gcaccaaaat cattccattt ccaggggctg cttgaRaagc |
| 65881 | cttgaacaga ttatttgcat ggagagaaat acttcccttt ttctgtttct ccctccctat |
| 65941 | tgcgggcatt tctttgtcat attcacactc cctgtttgta actaagaaga aaagtctttc |
| 66001 | tgtttatggg atcagcccta accacccaag tatcttcctc ctcaccagac ccagtgtctg |
| 66061 | ctgctgcctt tcattattca ctggctaacg ttaatcaggg tcccatacgg tacaaatctg |
| 66121 | aaaaaggaag cagagatgtg agcagaaata ccatccagac agtaagtgat aaacggattc |
| 66181 | acccacttta atgtgattcc ctctggcctc cacttcctcc atgcctctgc tttgcctcca |
| 66241 | ggaatagaaa acctacctgg ggaaggcgga agacaaaagc tagaggaatt tcattataga |
| 66301 | gggttaatcc atttatcagt tgctatttgg attgtttaat tggtttctaa gtcattgatt |
| 66361 | tccatcttca gggaggcaga caattctctg caagaagtgt tagctgtttg ggggacacag |
| 66421 | aggagctggc cacattctgg ggtctgttca gtgtaggctt gtcttcatca gtccctggat |
| 66481 | ctcaaacaca tttgcatggc atacagaatg cttttgcaaa taatctccaa aaagcagtat |
| 66541 | atctgcaaat attttactct gcctataaat tttcattgcc caaatccaga agtaccagag |
| 66601 | tccccttctc ccttctccct caattgccat cttagtcatt tttagaactc tcctaagaaa |
| 66661 | atccaagcat ctctatgaag caaataaact cagggtttcc tcaacaagat atggatggaa |
| 66721 | aactatcaaa tggtgaacag aaagaagaga tagggtatac taaatttagg ggctgagggt |
| 66781 | aaaccaaagt taagtgtcat gattgatatg agctagatat aaaatgtgga gatgccagtt |
| 66841 | gttacttagg tagcttcctg tatggaaggc aatcagagaa tcccatatat atcattcata |
| 66901 | aaaacttaca gggggcctag tctcctcagc tgtctctctc ctatactctt agggacttcc |
| 66961 | tataattttc aatttcctcc ccattcacaa gatctttaag caactaaaga atctgatttt |
| 67021 | ctccaacaca ctgtctccct catgacgtat gaggatgagt aagtaaaata tagagtataa |
| 67081 | aagcttgcag attaattctt gatatggttt ggctgtgYcc ccacccaaat ctcatcttga |
| 67141 | attgcagctc ccataatccc cacatgtcat gggagagact gagtaggata taattgaatc |
| 67201 | atgggggcag gtttttccct agcagctctt gtgatagtga ataagtctca tggcatctga |
| 67261 | tggttttaaa aggggcagtt cccctgcaca tgctctcttg cctgctgcca tgtaagatgt |
| 67321 | gcctttgctc ctcctttgcc ttctgccRtg attgtgaggc ctccccagcc aggtggaact |
| 67381 | gtgagtccat taaatctctt tgtctttata aatcacccaa tctcgggtgt ttcttcctag |
| 67441 | cagatgaaaa tggactaata caattctaca cagcattcac taccaacaga ccattggtag |
| 67501 | aacaccctat tgaatcagga actctaatac tacaagaaat aaaaaattag aaatgatcta |
| 67561 | gtagagctaa gcaggcagtg atggaagaat tcagccttcg taaaaagacc aaaggaaaag |
| 67621 | aattgaattc atctgaaaaa gggaagcttc tgacgagatg gagtctgagc agcatcctgt |
| 67681 | tggccataca gagttcctgt ccaaggggtg agggaggtcc taaaatccca tgtataggcc |
| 67741 | ccggtatgtg caggagtgac atgccaggcc agctggggtg tggaggtaca tttttagtct |
| 67801 | ggcaatggcc cagtatagaa agtgatcatg cacagctgtt ttatttttca gagaaatcat |
| 67861 | ttttgcttca gtttctactc tttcttacaa cagtgagctc aaagcatcct tccattgtgg |
| 67921 | tcagatggtg tggaatggac taattgctta tgcacctgat tttcttaaac attttggtac |
| 67981 | ttgggattag tttcagagca agggttctcc catggagctc tcccaaactt cagctaaaac |
| 68041 | atatcagtca tgccctgggg agggtactgt tgatatcacc attatgaccc ttgtttatat |
| 68101 | tgagtctgtg tgcctcctct ccccgtttca cattatcatc tctccatatt aaataggtct |
| 68161 | actccttcag gttggagacc aggtctaacc cgttgttgga acccctacaa tctgtgggat |
| 68221 | gagtgacttN agagagggat ggcagggatg ttagtgggtg gYacagtgat gccctgggag |
| 68281 | caccggatat cattcactgc ccctggttta tttccctacc agaggagagc tggtccatgt |
| 68341 | gtgcatgaat tggtctgtca cctggtctca tggccagctc ccagagtaca cagaagtcgg |
| 68401 | tagccaatgg ccctgagtca ctggaagaat aatactgtac ccctcctgtg ggaggcgaga |
| 68461 | catcattctg caccaaacac acaagaacat agaagacaat gattcattgt ctcttgagaa |
| 68521 | gaggatacct ggtcatgcct atccactcag cttccctttt gttcaactgc gtattgaaag |
| 68581 | gagaagaaaa ggaagcagac atttatcaca ccacaaaact gtggcaaatc ttttgctgga |
| 68641 | tgctttatat atgttatttc attgattctt gcaaaaactt tgtgaagtag ggaatttttt |
| 68701 | gacagatgca gtaactgaaa tccaaagaga ttaaagcgct ggctccaagc ctcggttggg |
| 68761 | ctcttcgaag gcaggtttca cccccagttt gtctgtgtcc ccagagattg tgcctctaat |
| 68821 | cacacttctc tgcctcctgg gaagtccaga ggtaaacact ttgcagccat accttgcata |
| 68881 | taaaatattt cataaatctc tttagagtca aYctgattcg cccaatatga tatttacgaa |
| 68941 | gcccaaagct ataaaaatta ttgagaccgg cctgataaaa gtggaaattc cctttgaact |
| 69001 | taaatttaat gtccctacta cagtaatcca tcctgggtga tcctgtttat tgtcatctac |
| 69061 | tctatgaaaa cacttctctg gagaattcca gaggctcatg aaggaatttt tttaagtaaa |
| 69121 | gctcacatca tggctaggtt ggcctatggc ctgacccagc catgagcaat ttcctggtac |
| 69181 | tgtatatgag atcattataa aaaagatttt actacaaata ggaagctcgc ccagaccttg |
| 69241 | taagtgggat ttaaatagag attttgcttc tgcttatggg aaattgagtc ccgctttata |
| 69301 | gaataagatg acggggaggg caaagacccg actggcatct ctatggcagc atctgcgatg |
| 69361 | atggatgtgg ttttctgttg attaatgttc atgggctgag atcatataag agatacaagt |
| 69421 | aagttgctga gaaaataaag tgagccttaa gagtttagaa atctacctga gggagataac |
| 69481 | tctaaaaagc tcaaaggaaa aaaaatggaa atcaggaaca aagttcatga agaaagtaga |
| 69541 | agaaaaataa cagaaggcag ggaagagcat tcagataaat caaagcaaga aaaatgaaaa |
| 69601 | gtgaagcttc acacagcccc atcaccacgc tcagggaagc gagagcaggc attctggaaa |
| 69661 | ggtatcagtg ctcgctacag gatttttgag ttaaatcagg ttttatgtct atggcaaaca |
| 69721 | aggccccaaa tcttccataa aactaccgcc caggcagcac cgcctctccc cctcccagtc |
| 69781 | ggatctccgc tgtggtgtRc ctgccactca cagaaagaat gactggcagg gacaaaccag |
| 69841 | ccgtcggcaa cagccactcg tctgggttct gctgagacca gagctgcgtc ccaagcatcc |
| 69901 | tagggaacag gaaactggcc agaagttcag gctcctcctt ggcacgtgca caaaagcatg |
| 69961 | gcctcacatc ctgagcagac gctctgacca cctgccaggg cctggtgtgc acaaatgcgc |
| 70021 | ggccacNctc ggtgcccacc acccaccacc cagccccagc cccagcccca gctgagcagc |
| 70081 | tccatttctg gagcctgcta gcctgggaag ttggggtgca cagcccttgg ggaattcggg |
| 70141 | ggcagtgagc agagccctct gggaagagat gcgcctctcc tcgcccctct cgtttgttcc |
| 70201 | ccccgccacc ctccacaggc tgacagttcc cgtgtccctc tacaggtctg acatgtttcc |
| 70261 | atggcaaccg gaatcctggg ctccggcagc ctccgctctt gccacctgtt atgacttcat |
| 70321 | ctctcaagtg taaagcatgc tagccccgtg ccacttggct gtgcagagcg catcttcaga |
| 70381 | ggacgacgga gggaaggaaa ggggagagga aaggagggaa agcagacagg cagcccaggc |
| 70441 | cggccaggcg ggacgtgctg gctaggccag tccagaggag gaagcttggg ggccggagat |
| 70501 | gagagggtgg ggagtaaggg tggaccccga cgtgccttcc ctgcagaatc aggagcaagc |
| 70561 | ctcagctgga ggccactgcc acagcctgct acctcggcct agcctttaat gagggctgcg |
| 70621 | ggaggaggga gaggcgggct ggggagacaa ggcgcctgcg aggagctccg gcaggcccag |
| 70681 | cgtgaagcag agccaggaga gggttaaaag tggcgtgata gagactgtgg ggaaggctgg |
| 70741 | accagagggc aaagggagca tcaaagccgg gataaatggc cttaattctc atgctcccac |
| 70801 | tcgctggtcg tgtttactca tataaattat cccccaggcc tgacaccttt tttcactgct |
| 70861 | tccctagaac tgatgaaaaa ataaatccca cagatcccag gaataaaaag tgtggggctt |
| 70921 | tcattcaggc tggttctcct tggccactaa tagcaactca ggtcccgccc cagggctcca |
| 70981 | ggaggaagac aaaggaaggc ttcatcccaa atgtggaggg gcagggggcc tggaagcctg |
| 71041 | gcggggaagc agacagtagg gcccgctccc cacaggaccc tccaatcgtc agagccctcg |
| 71101 | acggcagaga actcgctctc cccacaggcc atcttgccac cagctctgag gacaccgtga |
| 71161 | atatatggga aagtaaaggg gactacgctg gagcggatgc ggaaggcgct gaggccaggg |
| 71221 | ctgctgtcat gagtcctcac atttatggat ggggctgata aactgtcaca ctccgcaccg |
| 71281 | tcaaggacaa ggtgagaaga tgagctggaa tggcagcaca tgccacagga gagctggaaa |
| 71341 | ggcaggtggc taacaacgaa agccatggcc agagggcggg cttctctgga gaggcctctg |
| 71401 | cacacaggga agggccccta ggcccggggt tttgactgcg ttgccgatct agaggcaaga |
| 71461 | gaacacgcta ggtgatgtcc tgaggtccct ttcaggccta ttattcaagc attataatca |
| 71521 | ggtgaagaaa gagacagaaa agccaagttt tccacaccca gaggaaccca gcctttcatc |
| 71581 | actgttctat actcatgtaa atgcctcttt gcctgttacc catgtgctac ctgagggcac |
| 71641 | atgagtaaca cctgagggca ggaaccatgt ccttcactgc ggtatccctg gcccctggcc |
| 71701 | agcagtgagt gacacagagc tgacaatcaa tgctgagtga aaggatggac aaatcatcac |
| 71761 | agaggctttt tgaggggaaa aaaaatctgc caggcaaagg gaagttaatg actaggaatt |
| 71821 | gagtggacgg agagtccccc gagcacaggg tgaccatcct aagtccctct tatatcgggg |
| 71881 | cacagaagaa gtgggaagga gaaatattgg agccccttgg agagaagaaa gctgtggtgt |
| 71941 | ggaaagccac tgcatctacc tcccccagga atcctctctc ttaggcattt cttagtcaac |
| 72001 | cagaatggaa ggcatcaaga atccccaggc tcagctggcc ccagagtctt gagactcatt |
| 72061 | ctgtctctca ccagggactc agcaggagcc agtgagaaca gaggccccat cttgctgcgt |
| 72121 | gttttggtga ctgtagaaat aatataaata tttcaaaatg tggcgtcaac cctccaccct |
| 72181 | tcctatgcct gccttctttt cctccatctt tatccaatac ttacatcttt agatacaaac |
| 72241 | ctggtgaata tttactccca aaaactgtgc cataaaagca gaaaacatat ccactcacac |
| 72301 | tgatgtctcc atcctgagat ggatatattt ggtgagagtt gtaatttgcg gtttgccaaa |
| 72361 | aggccacaac aatttgccag cagtgtgtgc accatgtcac cagctctggg gctagatggt |
| 72421 | cacacggccc ccaaggaatg tcctttgtgc tccagcttta ggtgggcaac cctaggtctg |
| 72481 | ccattaatgc ttcggaatag taagaaaaag accaagtaaa gcttaggaat gggtcggata |
| 72541 | tgctggctca cgcctgtaat cccagcactt tgggaggcca aggcaggtgg atcatgaggt |
| 72601 | caggagttca agaccagcct ggccaacatg gcaaaaacct catctctact aaaaatacaa |
| 72661 | aaattagcca ggtgtggtgg cacacacctg taatcccagc tattcgggag gctgaggaag |
| 72721 | gagaatcact tgaacctggg aggcagaggt tgcagtgagc caagattgtg ccactgcact |
| 72781 | ccaggctggg caacaagagt aagactctgt ctcaagaaaa aaaaaaaaaa aaaaagctta |
| 72841 | ggaatgttct cagtgttcat gacactgaga tgaatatctc attacgtgac acgtaatgag |
| 72901 | atatttcagt tttccagtgt ttggtgatat gggtcagttc atgccctggt gtggcaactt |
| 72961 | gtgattgcta ccaaatgagt ttgttgaagg agaaaatgtt aatttgagag caactatatc |
| 73021 | aaccaggctc tgaacaaagt gatgtcactc cccttaatga attccagaca gcttggtgtg |
| 73081 | ttagagctgg atgagttttg tctgtttaag ttgtcgggat attattatcg acataattag |
| 73141 | gattcaacaa gccccttgtt ttgctttgcc cagccgtgcc ctctcaaccc tcttcactag |
| 73201 | caccactgaa ctctgtctgc aaggggacca gcattttctt tgttcttcag acagaaagcc |
| 73261 | gtatttaaga aaagggaaag catgagcctt ctgctttaga cctgaggatt tggaaggaat |
| 73321 | caagagacca cagaRtcttg gggcagcaca gcctgaggat ggtggcacac gcaggggcgc |
| 73381 | actccaaaag caggccggga agttggtgct gagagtcaga gacgtctgcc ctacatggga |
| 73441 | tagagcatta ggaaaagagc aaaaaccaag gctgcccctt caatttgaca gtacaaccta |
| 73501 | actcctggcc agttacctgg ctcagtgcat ccaaaagaaa aaaggaatta aaaggaagtt |
| 73561 | gactgactga acatcagtct tggaaagcta gatttctgta aggtagtggt ttacctggga |
| 73621 | acttgtcaaa aatgcaaatt cccagtcagg cacagtggct catgcctgta atcccagaat |
| 73681 | tttgggaggc caaggcaggt ggatcacttg aggtcgggag ttcgagacca gcctggctaa |
| 73741 | catagcaaaa ccccgcctct actaaaaata caaaaattag ctgggtgtgg tggtgcacac |
| 73801 | ctgtaatccc agctactcag gaggctgagg caagagaatc acttgaaccc aggaggtgga |
| 73861 | ggttgcagtg agccgagatc gcaccactgc actccagcct gggtgacaga acaagacccc |
| 73921 | atctcaaaaa aaaaaaaaaa aaaagcaaat tccagggcct gaattgctga atcagaaact |
| 73981 | ctgggggtgg gatccagtgg gtgggctgtg ctttaacaaa tgctccaggt gattctgatg |
| 74041 | cagcctcaag cttgagaaac atggctttaa ttggtgtgac agtcatgtca ccYggaagtc |
| 74101 | attcacttaa tctKcatcat agcctatgag atagatgcca ttaatgatct gatgttacat |
| 74161 | agtgatccat gaggcatatg gaggctatgc agctcagcta aggtcacacg ttagtcagtg |
| 74221 | gctcacttgg ctctgaaccc aagcaaccgc gcacctgagg ggaggtgagt cggagaggtg |
| 74281 | gacacgcagg gcctcacagg ctagggcttc ccagattttg ctgtgcaaat catttgggga |
| 74341 | tctcattaac aatcaggttg gaattcatag atccagggta cagcctgaga ttctacatgt |
| 74401 | cttaaaagct cccctggtag ggtggccacg catccaggtt tgactgggac agagggttct |
| 74461 | agggcgtggg gctttcagtg ctgtagctgg gcaaacctgg acaagctgtc accaagcccc |
| 74521 | aggtgcttct gaccacactg tcagatgcaa aacacagacc cacaagatga ggagtttggg |
| 74581 | ttgtattcaa aYgtgatgaa agtgttgaga gcatgagagt gacatcatct gctttatatt |
| 74641 | ttacatatat atatattttc ttagaaacaa gatctctctc tgtctcccag actggagtgc |
| 74701 | agtggcacaa tcatggctta ctgcaacctc aacctcctgg cttcaagcaa ccctcccacc |
| 74761 | tcagcctccc gagtagctgg gattataggc atgcaccacc acacctggct aatttttgta |
| 74821 | tttttagtag agatggggct tcaccatgtt gaccaggctg gactcgaact cctgagctca |
| 74881 | agaaatctgc tcagctcagc ctcccaaact gctgggatta caggtatgag ccacagcctg |
| 74941 | ctttagaggc tgggagacca atcaaaaaag ttttaacaac ccaggcagaa gccaggggtg |
| 75001 | gccagaagat agagagaggg agaaaatgta gggtatattt tggaagactg aaggaaaacc |
| 75061 | Ragaggcaag gatgaatcct aatatttcac ttcaggctca agcttttttt tttaaaagag |
| 75121 | caagtttaga agaaaaaaaa aatgagagcc aggaaagaca gtgagacaca gaatgagaga |
| 75181 | gaaagactca ggaaaattat tgaatagaat gatgtgtgat ttttttttct gtaggaaaaa |
| 75241 | aactcattta ccaattaaag tatgacctag aatttgtaat gcaggggtgg ggtctctaaa |
| 75301 | acatcccaaa cagcctaagt tagtaaagag ctttgaagag taaatgtgcc tactatggac |
| 75361 | tgaagtgtgt ccctcatccc caaaattcat atgtcagagt cctaaccccc aaagggatgt |
| 75421 | tatttggaaa agggtctttg gaaggcaatt aggttcagtg gggtcatgag ggtggggtcc |
| 75481 | tcgtaatgaa attagtgccc ttattagaca ccaaagagct tgtttctctc tctccacccg |
| 75541 | tacacaccaa agaaaggcca tgtgaggaca caatgagaaa gcagctatct gcaatccaag |
| 75601 | gagagagccc tcaccacact ctgaccctac cagaaccttg atcttggact tccagtctcc |
| 75661 | agaattgtga gaaataaatt ccKgttgttt aagccaccca gtcgatggaa ttttgttaag |
| 75721 | acagtgccta ttaacatgtt catctctttt gaaaagccac aatgcagcat gatgcattag |
| 75781 | acaactggtc ttgaggcaaa agttaaggga catcggtagg accatgacaa agatccaagc |
| 75841 | cttgggacta gggcccRtca ctcagggttc caaagtcctg acataactga atggtatcca |
| 75901 | actcaagccc attcacatct cagcttccca ccttgctgtc tgttactcaa cctctctgag |
| 75961 | cctcagtttc cttgtctgta aaatgagaat aataacatgt ccctaatgga cttatgatca |
| 76021 | taacatgagg tgatgtgtgt aaaatgcctg acacttacta gttgttccgt catctctttc |
| 76081 | cctctttcct cttctacgtg caagataatt ttatttctaa caaggcagga aaaatcaaag |
| 76141 | acctaaataa gtaaactctc ccaataagtg gtacatgact catgaattca cattcatcct |
| 76201 | ccctgaccat ggatacatga cttggtcttt gtgtgccttg ctgtcctctg ggggatacag |
| 76261 | cacctgcctt gcagggttat tgtgaggaat gaatgagata catgtggcat gcttagcacc |
| 76321 | atgtctgaca cacttaagga atgcaagcaa gtaccattgc cactgttgct gtttggtatg |
| 76381 | tgggtctttt caatcctgca catttctagg taattaggcc caaaatgcga gagaaataaa |
| 76441 | agagattcag agacagScaa gagacacgtg gagccccaat cctagggcaa tggcttggca |
| 76501 | aataaaaggg tgccacactt gtctagattg ccctccacag ctttctctgg ctgggtttta |
| 76561 | tgactctgtc ttatttccac aattgcacca gaagttctcc cagggctgag accacgttat |
| 76621 | ccctttctgg aggcccagtt acacttctcc caacaactgg gtgcatgtac agtaagtgct |
| 76681 | taataaatat tcataatgag cctgttcaga aggatctgtg tcccatatct aagatttagc |
| 76741 | caaggatatg ccagggccag gtcaggctct gatagttact gctgatgcca atgacacccc |
| 76801 | acaggcagtt agcttcacag tggtggcaaa gtcccatcac atgggctcag atggaaccat |
| 76861 | gattcagatg agtcatctgt ggtcacagac atgcctgtgg gcccaaatgg gacagagatg |
| 76921 | ggacattagt gctcacaggc atggtgccaa acattcacat tatctcattt aagactcaca |
| 76981 | gcaatcccat gaagtcagga ggattattac ccccgtttta tagatgaggc aactaagaag |
| 77041 | cagaagtgta ggtaagtggc ccacatttgt accactggaa gatggcagat tgccagggtc |
| 77101 | accattcaca ccctggactg accaactccW cagccattcc ctcagccaca gggtcaagga |
| 77161 | tgtagatgac ctttgaggcc agaaaatgag ccccaagtat cccctgcaag gaccagagca |
| 77221 | actctatgga ttctttgcta tataatctga aaactgtcta gttaaataat aatttctcta |
| 77281 | aagaacacct gttactagtt tccaaattta ctctttgaga aggaagaagc agcccaattt |
| 77341 | cccagaaaga gctctgtatc taaactttcc tctgtctccc ctaataacaa aggctggatt |
| 77401 | tctggtgtca agtttaggta cgcagttggc tgtgtaggct ttatggcttt tcagtcacct |
| 77461 | ttcagagtaa tctaaaattt ctaagcttgt ttcaaaggta attttcactc tatctcttaa |
| 77521 | agcttttggc cgccctcaag ccatatgtta gaggacaaga gatgtcagaa gactaacatc |
| 77581 | tgttagatct ttcccaacag tgtggcaatg acatcatatg cttcttttag ataatttgca |
| 77641 | cattatcaga ctaattttca gattatctaa tcagcagaca ttacccttaa tcaacttggt |
| 77701 | tctccccttt cagctctgca gagtacaaca tgccctccaa aggcgtgcta gagctggggc |
| 77761 | tcttttttct ctcaacagag gtgaaaagcc taactttgca ccaggcatac cacccttgca |
| 77821 | caaaaggctg agacctccag gaacctagaa gaacccatct tcaattccac agacttgaaa |
| 77881 | aatcagctgg aaatacattt tatagactca tattttttta aatttccctc ttaggagctc |
| 77941 | tgagtaYgct atagagagta tgttatattt acttatgcta ttaaataaaa gagatacaac |
| 78001 | atataacaat aagactccaa acagatgttt tttaagttat ttgtttttgc cttctagtaa |
| 78061 | ttgctactct ggcttcccac tgtctatagg aataaagact acacagaggg gcatagaaga |
| 78121 | cgcttcccac tgtggcccct gattacattt ccatctttcc tctgcatccc ctaaatccaa |
| 78181 | cattactatc agacccaaag acttccaatt gtctgaatgt gccatgctat ctcatacctg |
| 78241 | cctgccttca tgtacagttc cctctacctg ggaagccctc tccctcctaa gaaattcctt |
| 78301 | ctcattctcc aatggctaac ttgaatgtca cctcttcRgt gaagcctttg caaaccccct |
| 78361 | aatgcagtag tcctaaagct tgagtatgca ccagaatgcc ctggagagct ttgtaagacc |
| 78421 | cagactgctg gcccccatcc ttggagtctc tgtttcagga catccagtag aacctgagaa |
| 78481 | tctacatttt caacaaggtc ccaagggatg ctaatgccac acttggaata ccactgccct |
| 78541 | aaggcataga aagtaccctg tgcccttgag tcctgaactc cctcacggtt ctgtacacac |
| 78601 | cccattgcac actcccactc aagactgtga gctcaaagac agggctgagt cctatttatg |
| 78661 | cctgtcctcc ctagtcccag catatagaca atattcaata catggttatt acatggatgg |
| 78721 | gtggatggat agatggacag atgagatgga tgatggatgg atggatggat ggatggatgg |
| 78781 | atggatggat gggtgggtgg gtgggtgtgt gggtggatgg atggatggat ggatgggatg |
| 78841 | gatggatggg tggatgggtg ggtggatgag tgggtgggtg ggtgggtgga tggatgggtg |
| 78901 | ggtgggtggg tgggtgggtg ggtggatgga tcgatgacgt atacacggat ggatggatgc |
| 78961 | atggatggat tagactgaat ttatttacac agcttttcat ccaggcccag gacttcataa |
| 79021 | atgtctatcc tcactctaaa aattacctca aaaacatcat ctataatcaa ttcccactgt |
| 79081 | gcagagaagg taaaaagaag ttcaaagagt ttaacatcac aagcctatgt taagggggtt |
| 79141 | ttgaattcta gtccagagct ctttctgcta ctctacccca ctggggctcc ctaccaaggc |
| 79201 | tgcatttaag tagtcacaat ggtatgcttc tgagtgtatc tttcagagtc ctatcctccc |
| 79261 | cagctgccct cccttcattc atgtatgtgt acatctatgt gtaggtgtat gtgtgtgtac |
| 79321 | atatgcatgt gcacacacac agtcatttat ttcatttgca aaaagtaggt gaaggtggta |
| 79381 | aaagaaatgg aggccgaaga ggcacccagc ctcatgcagg gagagggtgg gatgcagccc |
| 79441 | acatgcagag gaccccatca gaaaagcact atttcccaga ggggagtgtt tgagctccaa |
| 79501 | gcatccctgg cttcctgtag ttgaggcaga gaggcaaggg cacacaggaa aggggacaaa |
| 79561 | cgtacaggaa acacaagaaa tgtatactca ggagaaaatg agagactgag ggagaaacaa |
| 79621 | aggcgYgaga gcatcagctt gggggccagg cgatcaggag tggcagtgga ttgctgagct |
| 79681 | tgcagcacag ggcagcatgg gcgggggcgg ggaaggtcag cgccggctcc agcagtccac |
| 79741 | tctgcccttt cagcagcagc accaggcgcg ggatgagcca tgggctctgc tgaagacaaa |
| 79801 | gctggggggc gatagagcaa gcgacagcag gaggaccaaa gtcaaaaggt cgctactgct |
| 79861 | gcaggtggtg caaacccagc tgcccttggc cctgcaaacc ctgtgtctgt aaggctgttt |
| 79921 | ttccattagg cctccgctca catgcagctt gggtcaccct gcagctctgc taaaggggga |
| 79981 | aaaagtcagc atttccactt cactggtgtc cagaccttct cctgggggag ccgcagatta |
| 80041 | gcctcttgga ttaagctctc ctacctgccc ctcccctcca aaagcctcac caagggagga |
| 80101 | tcacgaagct gagaccttag gatgctgccg agctgtcact tccaattcag cacacagccc |
| 80161 | cagtagcatt taggggcagg atggatggga gccaccccct atccccaaac agtgggagca |
| 80221 | gctaatgact aggtattaat gatgctggca attagtgctc acaagaaaat agtcgggggg |
| 80281 | ctgctcacca ggggaatgtg ggagggagca gagcacacat tcggggcggc gagcaagtca |
| 80341 | gcggcgtgtg tggtgcagag aacctgggac gaccagctgc ctcatgggtt tgtccagaga |
| 80401 | aattgggggt gggggaaagg atgttttctg caggcactaa aagcctttct gctgtaccct |
| 80461 | ccccactagc aaaactgggt taatcacaga gttttcRagg agggcactgt gagatgtcac |
| 80521 | taaaaaggtg tttgtgagga ttctggaacc ttcttcgtgt ggggaggccc cggatccRcc |
| 80581 | tgggaatacc tattattttc taggaggttt taaacccttg gccaggcaaa tgatcctttt |
| 80641 | tgaagagccc actttctcat cagcagggtg agaaagagaa aaaggggagg gggagttgga |
| 80701 | gtgtgtgcca ctcactgaca agcccacaga ggtgagaaca agccacccat gaaatttcgt |
| 80761 | aggcagacgt ctccacatct ccacgtaggg cgaggggaga caggcacagc ttctttccca |
| 80821 | agcaaggctg tgtggcctca aggggagaac actagaaggc caaacacacc cgcccacgag |
| 80881 | ggctcgtgag aagatccaca cccgagtgct tgtctcagaa gtcaaccaac cctgagggga |
| 80941 | ccacagggat cactggggag actgcctcct tctacttcgg gtctgcagca cacctccccg |
| 81001 | tgcagccaca aacaccaagc acatggacgg acacatatgc acatgtgcac acacgcatgc |
| 81061 | acacatatac aaaaacacac acacacgcag gtacacacac aatacacatg cacacaaaca |
| 81121 | agcacacaca tgcacaaaca acacgcacac acatgcacgc acacacacaa Mgtacataca |
| 81181 | caacacgtat acacacgcac acacaacatg cacgcacaca cacacacata cacgcacaca |
| 81241 | caSgcataga cacacacaca cccggctcat aatgaggcta caggagaacc aaggaaaatg |
| 81301 | agacacagac agacaagaag Mattccaaac cccagggcat gaataaagga acaaaatggc |
| 81361 | ctgacatttg agaagagata taaggagata agctagcaca ggggagtgag aaataaggtt |
| 81421 | accaagctaa taaacacttt agagagcgcg acaatggcga agaaacactt ttcagggagc |
| 81481 | aaccaaaatc aaaggagatt cttctcagcc tcctcatggt gggagacaac agctccgtgt |
| 81541 | gacctcttgg tcacttagac cagggaaaag aggagagaaa gggaaatgca agtattggct |
| 81601 | gcctcagtac aggtcagtat cttttccttt ttgaaagaca agggtgactg gaccaaggaa |
| 81661 | agctcttccc agagagaaga cctcagtgtt ggagctcctc aggccctggc aaggagaggc |
| 81721 | gggcaaggct cggggggcac ctcctgctct gctgttcagg ttctgtccag gtgacaacag |
| 81781 | caggtgagaa agcaagggtc tgtcgcacta aaggcttgca ccttggaagt gagcaaaaat |
| 81841 | tcaagaaggt gctgaaggct gaattgcatc ccctccccac tcccaaattc ctatgttgaa |
| 81901 | agcgtaaccc atggtacctc atactgtgac ttggtttgga agtagggtcc tcaaagaggc |
| 81961 | atttaaagag gtaaagttaa aatgggcttg tcaggataga ccctaatatc caatctgaca |
| 82021 | ggtgtcctta caagaagagg aaatgggctg ggcgtggtgg ctcacaccta taatcccagc |
| 82081 | cctttgggaa gccaaggcag gcggatcacg aggtcaagag atcgagacca tcctggccaa |
| 82141 | cgtggtgaaa ccctgtctct actaaaaata caaaaattag ctgggggtgg tgatgagtgc |
| 82201 | ctatagtccc aactgcttgg gaggctgagg caggagaatc gcttgaacct ggaaggcaga |
| 82261 | ggttgcagtg agccaagatc acgccactgt actccagcct ggcgacagag aaagactccg |
| 82321 | tctcagaaaa aaaaaaaaga agaagaggaa atgtggacac aaaagcagat gccacagcac |
| 82381 | gcatgcacag agggataact gtgcgagcgg acaggagaag gcagcatctg caagtcaagg |
| 82441 | agaggaaacc aaccctgcca gcaccgtgac cttggacctc cagcctccag aactgtgaga |
| 82501 | aaattaattt ctgtggttta agccacccca tctgtggggt tttcttatga gaaccctagc |
| 82561 | aaacccataa agaaggcaat attacgaggg gaaagtgggg tggaggttaa gggagacccc |
| 82621 | acactaatct gaaSaatggt cttggccaca ggtgtgttct ttatttcaaa tctttggtga |
| 82681 | aagtggccag tggacatcag ttgactaagt cgttggctaa cacgcatcct ctaatcctag |
| 82741 | agtctgaata ttttaaacgt ctgatccctt tcactgcatt gagttgttca ctttcagaac |
| 82801 | acctaccacc aaatcttctg aacaaaatga aaaacacatg gcctgtccac gaatattctt |
| 82861 | cactgagaga tctcagtgtg gcccacatct tcaagcaggc tctaaagatt ccattatccc |
| 82921 | atttccatgt tgagatttct gcaggcccag agaccataag ggatgttcta gaggaaagtc |
| 82981 | aaatggaaaa tgctaagccc ctttggattc cctaattcat aattggaagt tgaggtaaga |
| 83041 | aagcagagaa acggaaacac aaaatcttca ggatcatttt gggtcttgag agcccaacat |
| 83101 | cccaaacgac aaatccaccc agatcacatt tgccatttga atgctgaagc aaagaacatg |
| 83161 | gggtctccag gcagcatttc taaggcagga gagccctgta ccaaaatgtg atcccctccc |
| 83221 | ctctacaaca aaaccacagc aatagataag aatattctaa ggcacttcRt agccaagcag |
| 83281 | gcatttcgaa tgtgtgtctg aactgacaca cctattacct atgcccaacc cagtctgagt |
| 83341 | ggctggctct tcaggagtga gggaaaagag ttgtaggtct ggcatctgac agtggccagg |
| 83401 | taaggacttt ctcttctgct taacactcac cttccttcct ctgggagcag agcagggtgt |
| 83461 | gtgagcccag ccctgctatt gctaagccta gggttgggct gagctgcagg ggcNgctggc |
| 83521 | tggctggctg aaagggccaa cacactacag cacattgaga ccactcaacg gttgctacca |
| 83581 | gaagatgcca gcagtgagaa aaaggaacaa ggaacgctcc ccagcctgtc taacccactt |
| 83641 | cttcactgcc ccaacctaac aagtaggcac tcacgcctgt ggttctcatt tctgtggttg |
| 83701 | ggtggggacc gagtctcctg gtcttgagct tcatccccct cctcgctggc caggtgagtg |
| 83761 | tgagcatagt ggtagctgag ccccggtcgg ttcttgtagc gcttgccaca gactagagag |
| 83821 | gaaaagagga aaaaagggag agatacaaat attagcaacc tgtgcagcca ccgctatcat |
| 83881 | tctcccagac agtgatccca tccatccacc ccagagccag gcttccctta aaactgtctc |
| 83941 | ccatcccaac atccctttcc cccacccaac agaaccaaca cccttgtaac aactcaggtc |
| 84001 | atggaaagaa aaactgtaaa agtaaaatat gaggttaaga gctgtgtgag cactccaccg |
| 84061 | tttctagctg tgagacccag ggcaagtcac ttgacctctc tgtgcctcag tttcttccga |
| 84121 | gataaaatgg gaacaataac agtacttacc tcacagggtc attgtgataa gtttatctct |
| 84181 | atgcaaagta cttaaaacaa tatcaggcac tttgtgaggg taattgttat gacgttgaat |
| 84241 | tcaaagggca ctgggaatct acccaatcca acatcccatg acacagatga ggcaagtgag |
| 84301 | acccagcaac accaagtgac ttgcccagga gcacacaact agtgtggcag agccaccaga |
| 84361 | acccatgtct cctgggttcc tgtgcactgt cccttccacc accccatggt tggcctctgc |
| 84421 | aagaagtaga aaagttttat ctggataatc cttttgatgt ctcacccagc gattttgtgc |
| 84481 | cagaaacaca tgctgcactt ctaccatgaa cgccatggag ttaaagcatc cttggtgagt |
| 84541 | gtagtcacca agagcttgtg cccctgatgc cccagggcag ccaaatctgt aaaaggtgct |
| 84601 | ctcacaagta ctacctctca aatggggcag tttgagccaa gggcagcgag caatcgaaat |
| 84661 | gtgccttctt ggagccagga agaaagggca gcagttctgt tggttagatg ctcagttcct |
| 84721 | gggaagcaga agtctggcta aatggctcaa ggtcacacta ccaccctcaa cctccccaca |
| 84781 | tagggatcat tatctcggcc cctaggggga cggcattgct ggtagcagaa acctcgagag |
| 84841 | tcttcccagt gaaatgtcca tgccaacctt ctatgcttgt cccccagcct gggtccccat |
| 84901 | cagagcaaac caacctgctc tctgctggag tgcctgaaag ttacaaattc taaaagactt |
| 84961 | ctcccttgtg ctgacatgac cacgtcatac aaggaagcat aaatgcttca caaggtggga |
| 85021 | agagcacaac tttagcatca ggcctgggtt caaatcccag ttccagctgc tgaggttcac |
| 85081 | tggctgtgtg gtcctgagag tatacagaag tgctgtcatc cagtcatctt atccaatacc |
| 85141 | cttattactt ctgatagagt ttctcttcct accagattct cttggggttt ttcagtagac |
| 85201 | aaacattggc caaaataatg tcacctcttc ttccccatat ttacataaac tattttatct |
| 85261 | tagcttagat catttgtaag aaccaccaaa acaatatctt caaatggcaa aagcaagcat |
| 85321 | ttccaatgtt aatgaaggtg gatttagtgt ttcactgttt agaacgRtgt atctggctaa |
| 85381 | tttttagtct ttaccaaatt taagtagttc ttcttattcc tgYtatagag ttttattata |
| 85441 | ggagaatttt atcaactacc ctttcagcat ctactataat atgattctcc tttagttggt |
| 85501 | tgataaggta aattacgttg atttcctaat atttcctatt attgaaccat tcttgcattc |
| 85561 | ctagaatgca ctccatttgg tcagagtata ttatttattt gatatactgc tgcattctgc |
| 85621 | tttctaatac tgcatcttga atctgtacta agacttgagc tcaatctata atttttggac |
| 85681 | tatcttttat taggtttcaa ttttatggtg atgttgattt ctaaaaatta actgaagagt |
| 85741 | tttgctgcat gaaattggac aagctgcagc tctgtgcctt ttcttttatc tgaaaatgag |
| 85801 | gataacagta cttatcttgc aggatgggta aaggtattat aaatagcatt tgcaagcagc |
| 85861 | tctctcaata cccagcacat aatagatgtt caataaatgg taaatgaggg gcaagggctg |
| 85921 | tcagcatttt ttgctgcatc ttaggtactg gatcttcctg aaaagaggga ctttgaccct |
| 85981 | tgcctcatat tataaaatct ggcaaYtggt tggacttctt tgttaaggta agatgagagt |
| 86041 | ctgaaattca agttggtgac ccagtgacta taaattcaag gcccaatgtt tagctcccac |
| 86101 | ttataagtag aacatatggt atttgatttt ctgtttctgc attaattcac ttaggataat |
| 86161 | ggcctccagc tgcatccatg ttgcaaagga catgattttg ttctttttat ggctgtgtaa |
| 86221 | tattccacgg aatatcctga acactgggta cacacagacg taaagatgga aacaacagat |
| 86281 | gctggggact ccaaaagggg gaagggaagg aagagggcaa ggcttgaaaa cctacctatt |
| 86341 | gggtactatg ttcactaatt ggatgatggg tttaatagaa gcccaaacct tagcattatg |
| 86401 | taatatatgc atgtaacaaa cctgaatcta aaaaaataaa aaattaaaat aaataggcct |
| 86461 | atcattggct acttacattc atatattctc tcttcctctc tctcataaat attgKaaaaa |
| 86521 | aaataaaatt aaaataaaat aaaatagtgc tatgcatctt aattccatgt gactcaggaa |
| 86581 | gctggaccca aagaggcccc tgaggtgcac tctggatctg cttgtggctt tttctattta |
| 86641 | gctttttaga taatctgaag ttaatacaca gcaaattatg gggtcattgc atataaatcc |
| 86701 | atttagttgc caggcatact gttagtaagg aataaatgct tattaaaagt aatgataatg |
| 86761 | atatcagtat ttacacaaag aggcctcagc cacctctgca gatccccagt ataaactcac |
| 86821 | agcatttcac ttgtgccatt ttgttatgtc aatcatcact gttgcttaat cacacaattt |
| 86881 | caggatctta atggtcattt tgtctatcta gagaagtgtt ttattcccaa taactStaga |
| 86941 | aggctgttct ggacaactcc attgataggg aactcactac cagtcaaagc aggcatttct |
| 87001 | cctttagact ccttgaatta tggctatgtc tgcactatgt ctcttaagga cagtttcata |
| 87061 | tcaatttcat cttgatatca acaactccac cttatatgtt gacatataca tactacgcct |
| 87121 | tcaaaacata tttgttgagt ggctcatgaa tgaatacaga accattttgt gggaaggcca |
| 87181 | ggagatgggc gaagaggcaa RttgYagaat ctaatccttc tacccacatt aagctcagac |
| 87241 | taattgttct taaaagtcta caaaaccaaa cctaagcaaa aagctccctc tatctccttc |
| 87301 | cccaacatgg tagaaagtaa taatgtttag agcaaatcct tttgctagaa cagagattct |
| 87361 | caaacagtgg tccccagacc agcaccatcc acatctcctg gcaacctgtt agagatgcaa |
| 87421 | actcttgggc tccaccccag actgactgaa tcagaaactc tgggggtggg gcccagtgat |
| 87481 | ctagattttt aaaagccctc taggtgattc tgatgcacac taaagtttga gaaccactgg |
| 87541 | gctagaaggc tggatctatt taggcttggc tccaccaacc ctatgtccac atgcccttct |
| 87601 | tgatggatgg ccgtagcagg caggggcagg ataggcagcc ttgagacctt tgttcagtta |
| 87661 | ttagcagtta ttagcagaat caggaccaga caatacatgg aggtcccaga agccagactt |
| 87721 | cttcatgaag agccctctgg atggagctcc atttttaacc gggtatttgt accatgtcat |
| 87781 | tcttctatca ccaggtcttc aattcaagaa atatttatta gacacctaat atgtgcccca |
| 87841 | ccctgtgcca caagaaggac aaaaatgaac aagccccaga cccctccatg gaggaggtcg |
| 87901 | cagtctagca gagatctgca tgtaaacaag ccagggggag gtgggttgga tacagggtga |
| 87961 | gaccgtgcca ggcaaagagg gacatcaaag aggggacagg aaactggctt gagtgagtag |
| 88021 | ggtagggctt cccagagaag gagcccctga catgagcagg acttcagcag gcaaaggggg |
| 88081 | ctcaaagcac atgcagttat cacctctgca aactgcgaga agtctaacaa agttgaagag |
| 88141 | gtggagcggt gggggtggat gtgaggaaga ctaacaggag aggaagctga gcaaggcttt |
| 88201 | atcctgacaa tggggcagcc aggagaggat tctatcctga ggaccaagtt aagcagactg |
| 88261 | acatgttaga aatactgctt aagattgtag ggcagaatat ggatttgaag gtRgcgaggg |
| 88321 | gctatttcaa ttgtttacct gtgcaacaat gaggacttgt gcatggagag gaaaaatggg |
| 88381 | gtgggagagg ggttaatgag agaacttggt ggaaaaggga gtgagtaaga aagcaaggag |
| 88441 | gaacccagaa agactaacat ggccttcctg gccaggcctg acccagcccc acccacctct |
| 88501 | ccagcctctt ctgacaccac gttctgcctc cacacagccc ttctgctcgc tggccttttc |
| 88561 | gtttattata gatttctctt cccaccacag agtcttcaca caagctcttt cctctgccta |
| 88621 | gaatgtcccc atccccaatt tgcctgatta aggtctaatc atccctctga tctcagttca |
| 88681 | aatgtcctcc cccagggaag tctttcaaat caaaattaca accttagttt tttgccttcc |
| 88741 | taggactaat cacgtgtcaa tacagacagt ccgcaattta caatgctttg acttttgatt |
| 88801 | tttcagcttt acaatggtgt gaaagcaata cacattcgat agaaaccata cttcaaggac |
| 88861 | ctatacagct gttctgtttt tcagttcagt attcaataaa ttacatgaga tattcaatgc |
| 88921 | tttattctaa aataggcttt gtgttagatg atgttgccca actgtaggct aatgtatatt |
| 88981 | ttcaaagtag gctagggctg ggcatggtgg cttacacctc taataccagc actttgaggg |
| 89041 | gccaaggtgg gaggatccct tgtgcccagg agttgaagac cagcctaggc aacatggtct |
| 89101 | ctacaaaaaa tacaaaaatt acaaaaaata caaaaattag ccacgtgtgg tggtgcgcac |
| 89161 | ctatagtccc agctactcag gaggctgtgg tgggaagatc acttgagccc aggaagcaga |
| 89221 | ggttgtagtt agctgagatc acaccacggc actctagcct gggtgacaga gcaagaaaaa |
| 89281 | acataaaata aaaaagtagg ctaggctaca ctatggttta gtaggtttgg taggttaggt |
| 89341 | gtaagacatg cattttcgac ttaggatatt ttcaacttat gatgggtttg ccaggacata |
| 89401 | gcactaccat aagtggatga gcatctgtat gcatctctcc cactggcagc aagcatggca |
| 89461 | ggtcctgtgt atccgcNgtg ctgtgtcccg atgaccagca cagggcctca cttgtggcag |
| 89521 | gtgctcaatg tcagctacgg aaagcaaaga gatgcacatg tttctgacgt gggcaactgg |
| 89581 | gtagaaggat gaaggaggaa aaccagattt aaagaaggaa gaaaatgagt tgagttcacc |
| 89641 | taaagcttgg gaaagagacg tagattttgg agctggcagg gtggcaatgg atgttataag |
| 89701 | tatggggaaa agtgtcagga agagactgtg gagagagaaa agaggtgcct acacaggata |
| 89761 | gaactcagaa aacaccagga tcttaagaaa ctggtggaaa aagagaaact gattttttaa |
| 89821 | aaatttgttt agaaagggtt gagtgtggtg gctcatgctt ataatcccag cactttggga |
| 89881 | ggctgaggca ggaggatcac ttgaggccag aagttcaaca cccatctggg caacatagca |
| 89941 | aggccccatc tctgcaaaag acaattcaag attagccagg tgtagtggca tgctcctgta |
| 90001 | gtcccagcta cttaagaggc tgaggtggga ggatcacttg agcccaggag gtcgagggtg |
| 90061 | cagtgagcca tgattgtgcc actgcactcc agcctgggtg acagagattc tgtctcaaaa |
| 90121 | aaaaaaaaaa aaaaaaaaat tagtaattgt taagaaaaac tcacacagga gtgagcaaca |
| 90181 | taagccaaag gaacagggtt tcaagacgac agtaacaatg acaccaccag tcacaggtcc |
| 90241 | tgYcaacagc tattgagtgt ttaccacgga ccaggcccac tccaagtgct tcaagtaaat |
| 90301 | tgactcacag caacctgcat ccccatttta aagaaagatg aggagattct gcaccgaggc |
| 90361 | agagccacag taataaatta atcagatgcc tgagaagggt caagaaggtt ggagactaag |
| 90421 | agaccactgg atttggaaat tgggaagttg ccttagcaag aagagaatcc acaaagtcga |
| 90481 | ggagcacaac tgcagggtgc tccaagaaat gaggaagtgc tgatggggcc agctgtggga |
| 90541 | ggaggaaaga gggaggttgg tgggtggagg ggagcttagg atcagggcag accagggtgg |
| 90601 | agcctgtgtt ttaccagcct attctgactc ccaagtctta gtccttggaa tgatttcatc |
| 90661 | ataggggcaa gaggaagagt gaagacttcc aagagttatt ttggttggat tgcatcagga |
| 90721 | tgaaactgct tcatYacaat ggaaagaaac gtcttgaaat cctgctttga ctggcttaga |
| 90781 | aaagccagca ctgtttctcc ttctgtgttt aatcatgtct tcctgtcact gcaaacacca |
| 90841 | tgggccagct tcgtgagcct gtgacctgtg cttccagaca gggtccgagt tcagaaaggc |
| 90901 | cccacacttg gtttaatgcc ctgctgttgg catcttgaaa ttcttaatat ttattgaaca |
| 90961 | agggcccccg cattttcatt ttgcagtgga ccctacaaat cacgtagcca gttctagaag |
| 91021 | tctgttgggt tcagagtgga gatctgagac taagttactc caaatagctg acctgatagt |
| 91081 | ctgagagctg ccaaggtgaa cccaggtccc aaccagtcct gagtcaggca taaaattccc |
| 91141 | ttctgagata atcttgaaag tctggccaga gtgattagct gctcagtagt catcatctca |
| 91201 | gtgccatcaa ccacccaggc ctggcttcta gatcggctct gccctcagag aggcagaatc |
| 91261 | tgcagcaggt aagtgggcag gctggatctg aactgcctgg gttcaagtcc cagctctgct |
| 91321 | gttgactctg tgatcttggg caagttacta atcctctctg tgcctcagca tcctctctgt |
| 91381 | aaaacaaggt ggatcatgac agcacacctg ctccccgcgg tgagacccat atgagagaat |
| 91441 | gtatttaaaa caaagcccag ctcatgggga gtcgtggctg ttctcagctg ctctcatcac |
| 91501 | cactgctgtc aaagtggccc aacaaagaga ataaacccca aagtggggag ttatttctcc |
| 91561 | tctgaagtct ggagtggaat agcaaggaaa agagggtggg aaaagaaaca ttttgaaaat |
| 91621 | aaagtactgg aaccatctgc tctcctttta tcatgtcggc attctcactc tggaaatatc |
| 91681 | tgtcgtgcgg aatcagcctg agccccagga gcgctgaagc agcagagagg gtcatgcccc |
| 91741 | atcttcccac tccagtctcc ttccccgggg gcaagcgctt ttgcaccatg cacctctgaa |
| 91801 | gccaccaccc ctgggaccag tcagccctgc ctgtgcacga cctaaggtcc ttcttttcac |
| 91861 | cttggcaagc gtgcacctgg attttgtgtt cctggcatgg tgcacatgga gagcagtggc |
| 91921 | tgtgacaata agggaggtcc agcttgcctt ctctttctcc ttccccctgg ggcggatcca |
| 91981 | tctgtcaagS tgcagaagga gtgtttgccc acttcccaat ccagaactgg attctgcatg |
| 92041 | caggacctcc tgtctggagt tatggatgcc ttcagtgccg ctccaaaata tttactgcac |
| 92101 | gccccctgcc ctccttcaaa cctaccccac tcctagtctg ggcgggggtc ggggggagca |
| 92161 | gggaggagga ggaatcaggg gcagcttgtg tgaacaaagc cgctgcagca gacagcagaa |
| 92221 | tccttcctgc cggggagaag cggctgcctt ccccactgat aacttaatga ctctggcaca |
| 92281 | ttttcatcag cccaacttgg agcacacagt gggaatctga gccagtggtt tccgagagac |
| 92341 | tgaaagcagt gactgaaggc aattacagga tgagcatccc agagacaaag cctgggccct |
| 92401 | gggagtcacc agccagtcac cagccacagc tcattcccgg ctctgccccg tggcactgac |
| 92461 | atacaaccct gaggacaccg cttccttctc tctgcagctg gacttcctca tctgtcaccc |
| 92521 | agaccccacc atgcctgtca tctgccgatg cctccaaggc atcacaccta ccatcaggca |
| 92581 | gcacccattc ccatgcagaa tctcacagct caggaggcaa ggatccccat tccacagatg |
| 92641 | aagaaactga gtctggggga gtttaagcaa ttctctggat ggcacagcca gagtttgagc |
| 92701 | cctggtctgt ctgcccttga agcacgtgac tgaaaggtgc gctggtgagt ctctgcttgg |
| 92761 | cactgcctcc ctccccaccc accccaacgg cctggggctg actggcgtgg cctgcttcac |
| 92821 | ccatgctccc ttgctctcgg caagaaggag gcaccagcag gagaacggag ggtgggaaRR |
| 92881 | aaaacacgtg gggacttggg ccctgccctg cctgggcact gtgctataca ggggctgtat |
| 92941 | aggggcccac ctccaagact ccagtcctgc ctggctctgg gaaccccatg tctcccctgc |
| 93001 | tccttggagc tggggatagg gagggcttcc cgctctccct gtcagcacct tcatcttgcc |
| 93061 | cacaccttca taatcactct ggcatcaaat tcccttccat ccagcccttc tgagtggaat |
| 93121 | cccacatcct gccggatacc atttggacac atattcacac cctaccagac cggctcgtag |
| 93181 | aagtttgggg ccaccaacac ctcccaacga ccctccatct tctctctgca gaaccatctg |
| 93241 | ggggatggag gcattcctac aaccaattcc cccgagcagg gagaagcccc aactgagaaa |
| 93301 | agccctgtgt gactcccaga tacaaactca gatcgggata aagcccagga ggtacttcca |
| 93361 | aaagccacct gctcagactg ggtacRcagg gtacagKtgt tcatcccaca gccacttcaa |
| 93421 | ctggccaggc ttttctcaaa agccaacggt ggatccaacc cccaggtggg cttctcctcc |
| 93481 | ctcccctgcc cttgaagttc gaggccagca gactcggtgg ccttctagag accgggggtc |
| 93541 | tggctctggg tctgatacta acccgctatg cacccttgga caaattcata cctttctggg |
| 93601 | tcttgatttt tcttatctgc agaatatggg ggttggatca gatgatctct aagtcctttg |
| 93661 | cattctagca cttcacaacK ctctgtaggt cctgaagcta ttatctcctc aactgtgata |
| 93721 | ggaaaatgat gaggctccca gaggctggct acatccacct ccccccaccc caccctgcac |
| 93781 | gattggcagc tgccgtcccg tggcttcata ccttctttca ggccaccctt gatgcttatt |
| 93841 | ttaagctgaa ttcctttgga tggagacaca ggggattggg ggtaggggtg gggtaaaaca |
| 93901 | ccctgatttt tcacagccac atttcttggg gcctggtggt gacccaggat ctatcaaact |
| 93961 | ccagtgaccc aaaagtcctg tgtcttcatc aagggagagt gtcctgctta ttttgggaat |
| 94021 | ttactagatg tgaggaagaa cagagacaag caaccagact ctcgtttcaa aatattctat |
| 94081 | cacctgattt ctggatcctc agcagggaaa tgcagaaacc ccccaaaacc aaagcactcc |
| 94141 | atgtgttatg ggaatgtttc ctgccctgct gagaagccgg gtaacagaat gcaggtgaac |
| 94201 | tcgggagtct gccttcttgg gtcagctgat ttgcaatgtg accttgggca agtgacttag |
| 94261 | tctctctcag cctcggtttc ctcgtctgta aaacaagggc aataacagtt tgtagctcat |
| 94321 | gggggaggtg tgagaactgc aggagttcat cttcttacaa tgctcagccc agtgcctggc |
| 94381 | acacagcaca tgttcaacaa acatcagcta tttttgatca ttcatttaac ttgatcagac |
| 94441 | aactccctta gatcctgcag cagctagggc acagaacaca actggggccc cagcttaggt |
| 94501 | gttaaataag taccttttct tgattcagta taaagaaaat aaacatttat tgaaaatatc |
| 94561 | tatgaaccaa gcagtgagct aattgttttt acacaatttc tcattgaacc ctcttggaaa |
| 94621 | cccactaagg ttaatattat tgtcctcacc taacagacca gaaaacaaag gctcaaagtg |
| 94681 | gttttgtgat ttgcctgagg tcatggctag gaagtgacct ggtaaagatt caaatcctag |
| 94741 | ctttctgact ccggagccac cttcttccaa ctggcatccc tttcagggtg gtgtctttga |
| 94801 | ttcctgattc tagtgagtcc tgcgttagca tgtccttaat gagcttaaat taagagtgaa |
| 94861 | actgaattct cttttcaatt gtacaataaa aataatttaa ataataaatg tcattgtcaa |
| 94921 | catttctggg gaaggtaaaa agagaaaggt caaaaatcag acctgattac cctaagctga |
| 94981 | ggcagccccc aggcatctgg gaatcctgct agcatcctct ctctcaatat ctctgtacac |
| 95041 | ctcccatgcc aggacatcag tgcggcctca aatgctgagc aggttttatt tgtgagttcc |
| 95101 | aggctatcca gtgtgattcc ccaccccctg cctgaggtag gccttcttcc acctacaatg |
| 95161 | tcagtgtaag ctcctcagtg ccttagagga aacaacaact gtccattagc atctgttaag |
| 95221 | acgtttctgc ctggattcct gccatttcca ccaactcata tgtcagtttc atcacactcc |
| 95281 | caaaattact tcccctttta ggatattcca gttaagctac cccttttgtc tcaatgaccc |
| 95341 | aagtgcaaga tgtcaagagt catcactgcc acctccctct ctcctctcct gctctccaca |
| 95401 | tcctgtccat catctgccgg gttctgtgaa tttctctttc attaagtctt cttcatctgc |
| 95461 | cccgtctatt tcactgctgc tatcaccacc atccagattt catgtcttct cctagctgac |
| 95521 | tagaaaaggc tcttctcggt catcaggctt caggctttcc tccctgaaca ccgaatccta |
| 95581 | cagaYcaacc ttcctagaga tgtccctagg attgtcatga attaccacgg accatgcagg |
| 95641 | ccaacctcta agccaggggt ccccaacccc ccgggctata gactgacacc tggactgcac |
| 95701 | agcaggatgt gagtggcggg caggcaagag aaacttcatc tgtatttaca gctgctcctc |
| 95761 | atcactcgca ttaccacctg agctccacct tctgtctgat cagattagat gtcacacKgg |
| 95821 | attctaacag gagcacaaac cctactgcaa actgcacatg caagagatct aggttgcaca |
| 95881 | ctccttatga gaatctaatg cctgatgatc tgtcactgtc tcccatcacc cccagatggg |
| 95941 | accatctagt tgcaggaaaa caagctcagc tcagagctcc cactgactct acattatggt |
| 96001 | gagctgaata attatttcat tatatattac aatgtaataa tcatagaaat aaagggcaca |
| 96061 | ataagtgtaa tgtgctcaaa tcatcccaaa acctgccctc cgcccctggt ccgtggaaaa |
| 96121 | attttcttcc atgagtccct ggtgccaaaa aacttgggta ctgtggctct aagcacctca |
| 96181 | gcctgatgtt cccagcaacc ctSgtctaac tctgtgcccc caatctccct acccacatgc |
| 96241 | tctgggttag tcgagctgag ccttcttctt ccctggtatg cacatcccac attcccttct |
| 96301 | ccagacattc atcaacagtt tggaagcacc actgtgctgg ggctgggctg caagttgatt |
| 96361 | ctggcctggg ccctgccctc agaaggctca cagtccagcc tgagggagca gagaaacaaa |
| 96421 | ctgtcatcac acaatgtggg aggcagccta ccaggaggta ggcacaaggt accagcagct |
| 96481 | tccagaaagg gacagtgata gtgtgtgtgg tgggggaggg aaggagaatg agagagagaa |
| 96541 | aaaaaaagag agagagagag atgaatgact gggatcaaat cttggctcta gcatttattc |
| 96601 | actatgtgac ctcaggaaag ttaattaacc cttcctctcc ccagtttcct tatctgtagg |
| 96661 | atagagatga ccaaagcatc catttcatag cacttttgtg ggagttaaag agttaatata |
| 96721 | gatgaagcca acagaatatt ccaggtctat aatataagtc aacaaacttc agcattatca |
| 96781 | ttattacctc ttccccctac ccctgggcac acccatccac ccctgggggc caatatcaat |
| 96841 | tccatccatg cgccctaagg gccttcttgg catcccgggg ctgcactctc tgacacagca |
| 96901 | gcccctcttc Rtgtgaccac tccctgccct gttccttcag tgctcactgt cccctcaatt |
| 96961 | aaataccgcc ccggcagtgt gctatctcct gcacaatatc agcctttctc cttccaagca |
| 97021 | tattggagaa agagaaaata agaccaaggt gggggaaacc tttgtcaagc acatgccacg |
| 97081 | tgcaaagctt tttacataca tcatcacagt cagctttccc actgtgcccc atccccacca |
| 97141 | tacaccggag gacatggaga ctcctacagc aaaatgactt tctcgatgtc acagataaca |
| 97201 | agcagcagaa ccactccaaa gtcagggcac atcctgcttc acccagctct ctgcaacctg |
| 97261 | gcagacccat caggctcctt gctttcctat actccccacc atgactcaaa ataagagaga |
| 97321 | tcaaaggcca tgcaccaggc cctgcagcct cacaactgta cttctaggaa acaataaagc |
| 97381 | attatgcaaa ggtatagctg gaacagtgct tttttttttc ttttttcttt tattattatt |
| 97441 | atactttaag ttttagggta catgtgtaca atgtgcaggt tagttacata tgtatacatg |
| 97501 | tgccatgctg gtgtgctgca cccattaact cgtcatttag cattaggtat atctcctaaa |
| 97561 | gctatcccta ccccctcccc caaccccaca acagtcccca gaatgtgatg ttccccttcc |
| 97621 | tgtgtccatg tgttctcatt gttcaattcc cacctatgag tgagaatatg cggtgtttgg |
| 97681 | ttttttgttc ttgcgatagt ttactgagaa tgatggtttc caatttcatc catgtcccta |
| 97741 | caaaggacat gaactcatca ttttttatgg ctgcatagta ttccatggtg tacatgtgcc |
| 97801 | acattttctt aatccagtct atcattgttg gacatttggg ttgattccaa gtctttgcta |
| 97861 | ttgtgaatag tgccgcaata aacatacgtg tgcatgtgtc tttatagcag catgatttat |
| 97921 | agtcctttgg gtatataccc agtaatggga tggctgggtc aaatggtatt tctagttcta |
| 97981 | gatccctgag gaatcaccac actgacttcc acaRtggttg aactagttta caggggcagt |
| 98041 | gcttttaata gtaaacagat attagactgt ctaaacatct aacagtagaa cattgagcaa |
| 98101 | attacgagac atgtccataa gctgtggcac atctgcataa tggaacacta tatagcccta |
| 98161 | aaaactatgt cataaaatct atttattgac acagaaagat gttcctgatg tatcactgag |
| 98221 | tggggcagMg gggcKttaaa atggtacgta tcatttctta gtcgtcttct gtgtgcgtgt |
| 98281 | gtgtgtgtgt gtgtgtatgt gtgtatagac agagacagaa ttaatcctag aatggaatac |
| 98341 | agcaaaacaa atacagcaaa aaattatctc caaggagtga gattatgggt catttttgtt |
| 98401 | ttttcctcta tttttttttt tttgagactg cgttttgctc tgtcatccag gctggagtgc |
| 98461 | aatggcgcaa gctcagctca ctgcaacctc tgctttgcgt atctgtaatt cctgattatt |
| 98521 | ctgcaatgaa caaatactat ttccatagaa aaagttattt taaaaaaata atgaaaagca |
| 98581 | cctctacaat ggttaatatc taccctatta tgggtcatct acacagaaat aggaattgtc |
| 98641 | atgaagtgtg catcaagaaa cagttagtgg agagcaaggt tgtgactgaa tagaaaataa |
| 98701 | ctccagaggg tgtgggtcac agcccagaga aggcaggtgt aggttagaag gtcaaatgcc |
| 98761 | aagatgcgag ccctggagga gaggaggcag agacaggagg gtctagggac agcaaagaga |
| 98821 | aacttcaccc tctccccagc cctgcctctg gccagtcctg ccggaacacc tgcaacctgt |
| 98881 | gtccatgaca caatggcatt taaatattta aacagaccca gctttgctgg aacggcgcac |
| 98941 | tcagtctcca gggatcaatc ccatgccaca agccccgctc cacaaatcaa gtttctgggg |
| 99001 | gcaccgagcc tttcctcccc tccccgatcc tacccccatc caaggacctg caagacaact |
| 99061 | gagtgatgag cccagcttcc cttccgcgac aaaggcagtg agcctgaagg ccgcgttatt |
| 99121 | ggcttttcca ctgcagcttg aagcgaaaag cacgtgagcc ttaaagaata tgYgccttaa |
| 99181 | tgccctcaaa aaaatttttt tcttagcaaa actgcaagag tcatctaact ctccctgctg |
| 99241 | cactcaggta ggggtaaaac cagggtagtt tgaaagaggt tgaggtcaag cagattttct |
| 99301 | gggactgcct tgggctcaag cttttgtctg tccagtcagc agatccaccc atcccatcgc |
| 99361 | acctcttggc ctgtggcaca gtttcccgtg gtattcccca atccactgct cccttcatgt |
| 99421 | cctccagaca tcttgcctcc ctagtaaaga gcatcattgt taagctgctg cctgggaaga |
| 99481 | aaggcgccac tgtgtgggga aggagtgctg atcagggcag tccggaggcc caaaggagga |
| 99541 | gccatcccct cacggacaaa ggaggaaacc agtgccatag caactgaggt gtagaaccag |
| 99601 | gactaaacgt ccagcctgct cctcccatca gcctgatacc cttctctctg aagtacacca |
| 99661 | ctgccaagaa acaagagact ttagaacggg aagagaccct agaccctatt ctgatactac |
| 99721 | ccccactggg tgtggcaaca cactccccga ctgggtttct agaagggagg caacagggtg |
| 99781 | aagcggccac acagctgaag acgcacatct gtagggtgga ggtggtagag aacagagact |
| 99841 | ttgctgagac agaggtggcc ttgagagggc ccgctcctgg tgtgtgacac ctgacgtgac |
| 99901 | tcaggagtga gtcatgcttc taggagggct ttagccatgt ctatttgcct tttgtcacta |
| 99961 | atctagccta atgccgtagt tggggggcag tgattttaat agatttaaat ttaaaagact |
| 100021 | ggaaatcata taattccttc catcctactt caattgtaac tactacctga ctccagcctg |
| 100081 | cccgcctgat gcctagaaat tgcccatcga cagcggccag ggcttcacct cataagtaag |
| 100141 | ttgtgcgttc tcattaatta actttgaaaa tgctttttcc gcagccttca agagaatgaa |
| 100201 | actccagggt aggagagcat atgtaagggg cagttagtat ggaagatgtc ctagggtatg |
| 100261 | gcggttctag agttgaggac gctgggatca tatcccactg ttcgcatcct agatcccagc |
| 100321 | tgtgtgacca tggacaaggc cctacacctc tctgagcctc agtttcttca cggtaaagtg |
| 100381 | aggtcaactg tccctgcctc tccaggttgg tgtgggggtt tacagaaata atccatgtga |
| 100441 | aacacttagc ctagcgctct acacacccta agcacctact cacaggaatt aagaccatga |
| 100501 | ttcaRaaaac acttcgagga aaaaagaaaa cttcctacac tgcttccctt gtgtctagca |
| 100561 | aaagggcata tgcctgcttt gctcctccct gcatggctaa tgcttagaga caaaatgttc |
| 100621 | tccctgaata tcacaacgtc atcaccaact tctttgctat gatgtttctt gaacaaagaa |
| 100681 | aatggaaaag tgggtctctc atcaggagtc tgctccaatg acgctaacag aaaaattcgg |
| 100741 | gcttgggagc aattaggaag agaagaggca aactcttgaa atgactttta aaactttcta |
| 100801 | gcagcccaaa cccaaggatt ccttccacct gcctgtcccc agttatcatc ctgcctacaa |
| 100861 | aatggggcta aggatactgc taggatttaa agcgattaag tgaattaaaa atcaagcgct |
| 100921 | ggatagctga gttgtatatc aatgtgaaat aaatggaaag aatagattct aaggagacca |
| 100981 | actaaaacac tgatgcaagc cagtggcaac ctgcctggag aattcctgga gactccagaa |
| 101041 | tgtccagatg caaaaagcag aaaataataa gagctgagca gtcaggagcc agaagccttc |
| 101101 | atgtttaaat gaagtagact tgtcactgtc aaaagccagt atacacaaga atcccctgaa |
| 101161 | gagcgctcag aaaatgcagg ccccttggcc ttccctccag tgattctgat accggaggtc |
| 101221 | tgatgtggag cccaggagtc tgaattctaa gaagcgtcta ggcattgctg tgcagctggt |
| 101281 | ccgggcctac actttcacaa acaccaacag acttttccac aatgtcctat tcagttgctt |
| 101341 | tttttgtgca taagaaacac ttttccttaa gcaagagatt aaaagggaag aaacagaact |
| 101401 | ttaaccctta agcttggtaa actttcaagg taattaaaag agactggtta cttgctggaa |
| 101461 | aacaaagtat tcatacatcc atacaccatg gattgggtaa aacataaatc atgttcaggt |
| 101521 | gatttaacat ttctctgaga cggaagaaat tcaatgactg agtaaaaggc atcaagcaaa |
| 101581 | ctctctatca cctgcagtta gcagctacaa taRtattcaa gccacccatt ttgcaaagga |
| 101641 | agcttggagc aagaacacaY aggaccacta tattcaggaa Kgcattccag gaagggttaa |
| 101701 | ataaatcaca tcagaatata gcttattacc actacacata aacccagaaa ggggttgggg |
| 101761 | tccaaggtga tctaatattt tcataaataa cttgaaagat ggagtgggaa ttatgcaaat |
| 101821 | cacaccatag atgatacaaa aatggcaggg acggcaaatc tgtggagaat gaaatttaac |
| 101881 | tactgggcaa tgtctttaaa tttgccttta ttgaatcttg tatattgaag tcacatatta |
| 101941 | ttggatctgt ttattgagac tcaatgggac caaatgcaga ctgatttatg gaagaaggaa |
| 102001 | taatcaaacg cataagtaga aaagagcatg cacctggaga ggcatcgaca ccgaggctgg |
| 102061 | gacaggctcc gactagtaag ctgttgagag ggtggacagg gcataagtaa ggagatatgt |
| 102121 | ccgctgctct ttgagccacc tggacttgat gctacttctt aggaRtgcca tgcttaagtg |
| 102181 | tagacctgga agggaattcc aagcagggtg ccatagagac tatggggtgg ggccctaagt |
| 102241 | caaccaagaa tatatgggat gttcagaagg tctcccagac ggacaaagcc ctaatgtgcc |
| 102301 | tattcgtagg ggaaagatgc gcaaacgaca aggggacctg taggagagaa aatcttcagc |
| 102361 | tcctattaag gaaaaacact cattcgaagc acagctggca tcatggaaga aagggcagct |
| 102421 | tatggtaaca ccatccctag ggacgctcga ggaggtggcc agacacctct ctacttagga |
| 102481 | tgcattagag attattaaaa gcactccttg cccagtttgg gggtggtggg ggtgggcctt |
| 102541 | gtgacctctc caaaggtcat ttctatccca aaggatcaca tgtctgaagg gcaccagata |
| 102601 | aaaataaagt ctattaatca ccgagctgcc acacaatttc caaagagagc cagaacagag |
| 102661 | aagggcaatc gacggtggcc cctgctccac cgagagaggg agaggaggca gcacgcatgc |
| 102721 | ggacagagcc aagaaagcag ccgctgagac ttccagaaag cctgccgtca ggcctcccag |
| 102781 | gcactacagg gcacacagaa ggtgggagag agcctgcggg gagggcatcg agagaagcaa |
| 102841 | acgtcactga gagtcccgaa acaaagccat ggggactcag gagacagtgt gtgatgcaag |
| 102901 | cactttgcag aaagaggggt gcccagtaag agagggatgc ccacaaacag cacttcatgg |
| 102961 | agatgatcca caaggaggca gaagtcagag gcaaaggtgg gaggagagga actgggaggg |
| 103021 | gatgaagtca gagaaaaggg cacacaggtg tgtgtgtctg ggagtccatg ggattatgat |
| 103081 | gggctgggaa tccaatgaaa gctgcagaga tagagcaaaa agggatccca cggtctatag |
| 103141 | tagcagacaa ttacatacga agataaagca ttttaaaaga ggaacaaatg gctgggtgca |
| 103201 | gtggctcaca cctgtaatcc cagcactttg ggaggctgag gcaggaggat cacttgaaac |
| 103261 | caggagttca agaccagcct gggcaagacc ccatctctac agaaaatttt aaaaacttag |
| 103321 | ccaagcatag tggcatgggc ctgtagtcct agctactgcg tatgctgagg caggaggatg |
| 103381 | gcttgagtcc aggaggtcaa ggctgtagtg agctataatc ctatgattgc accactgtac |
| 103441 | tccagcctgg gtgacaacgc aagagcgtct cttaaaaaat taaataaata aaaatgaata |
| 103501 | aacgaagaat atagtagcaa tgccccagtt ccaaagcagt tgttggcatt cttgatagat |
| 103561 | gtgctggtca agccaaacac agctagaaag cccaagacgg tctgagggcc cttcccagcc |
| 103621 | catggcttac agccagtctc tcaagggtca gcatgctggg gcttctatgt atcagagtga |
| 103681 | aactgaacgc catgtaggaa gcaagaccca ggccaaggca ggcacctcct ggtgctgcca |
| 103741 | aaaggcatca gaccccatgc ccYgctcctt cctcatcctg gactagaact gctttggggt |
| 103801 | ggagacgtta cctgcaggag tcatctcagg aatggcatct gcatggagta ttaagcagcc |
| 103861 | aaatgggagt catggagttt gagttcttct agcaaaatgt tctccaacca gctgtcaccc |
| 103921 | cattggacac agaacaactt tttgaaatca aaagaggcca gtgaaggggg agacaaaaga |
| 103981 | caccaccccc agggtgctcc ctgggcccca cccctacccc gccttgccag ctctctccct |
| 104041 | gccttcaggg agactgcaga ccctggcgag atgggtggga cacccagagg gagacccgtg |
| 104101 | ccagggtcca gcctcgactg tttcactgcc atcctcgctg cagcctcttc cctcctatgg |
| 104161 | cgaagctctg catcctcaga gaggagaaga cggcaagtga agatcattag gagtgtggtg |
| 104221 | gtgagggctg ccctcccata gaatgagcaa atggtacccg gtacagtgag aagatgacta |
| 104281 | gggcacaggt tttcaatctg cctcccagtg ctctgctgtc tggagctgac cacctccctc |
| 104341 | atcaagctgc aggtttcccg aggtccaccc actgccctgg ctccctgcga ctcccctccc |
| 104401 | gtgggctccc tagctctgtc tgcacgccct aaactcagga gccagccagg cagtcgcttg |
| 104461 | tccaaaagat gtttctgaag caagcaagaa aggctggaga tagattgagg gccatttatt |
| 104521 | aaagtaagtc atcgtcatag aaagattcat aacttgccct acctaggaca ggagaggaag |
| 104581 | agggaaagga aattaaaggg aaacaaatga aaagccatgg cgagccaagt acctttgtcg |
| 104641 | gagggttttg agttatgctt ttgtttgtaa ctctctgaaa tacagaaaga aatagaggat |
| 104701 | gaatcttaaa ggagacagag cgcgtgagag agagagccgg aggagagcca cacaggaggc |
| 104761 | atggcaggag gtgggggtgc agaactgagg gtgggggcca ccccagctgg cttcccctct |
| 104821 | gcagaaaccc cctggggcag cagaggccca aggaagtggt gactgcggtg gaggcggcag |
| 104881 | gcgagtaagc tggggtgcag acagaggaag aggagagcac acactgaggt tggcaggcgg |
| 104941 | atggccaagg aagcacaggg aagaggggcc ctgggggcag gcgcacaggR aggaaggaag |
| 105001 | gtgggaccgg cccatactta ctgtcacaga cgtaaggttt gtcgtggtct tcctgagagg |
| 105061 | cggcgtcgtg cctcctcctg ccccctgcag agccgcgagc ctggggagac attggggtac |
| 105121 | aggatgcttg ttaccatggt catcactaca gctccggagc atgcgctctg cgagctcctt |
| 105181 | acaccgtctt catcccaact tttgtgccaa caccagtccc atcttacagg ggaggaaact |
| 105241 | gaggcccaga gaggtcagga gacatgcccc caaatcactc agttgctcac ctgtatcacc |
| 105301 | tcattaaatg ttcacaacKc caagaggtgg tttctactag ggtccccatt ttacaaacca |
| 105361 | aaggactgaa gccatagaga ggttaagtag cttgcccaag gtcgttcggc tactcagggc |
| 105421 | agctgtactg aaaagccctc ctctacMaaa ttcccacaca ctaccgtgcc Rctgaggccg |
| 105481 | cttcctcctg ctccttagcc tcctagcttc actgRcacag ctgaggttga caaccccagg |
| 105541 | cccctcagca gaccagctga tgcacgtatc aggagcaagg cctgacgggg gcaaggcWag |
| 105601 | gcttctgtgt ccatccccgc agtgtcgcaa gagccagaca gtcctgacat ccaggcccag |
| 105661 | tgcacgagtg tagMtacagg ggagctcccg ccaccagccc atgcttggcc tactcagtag |
| 105721 | gggtgggctt tggctaaggc ctccctgtgt ggtctgcctt gggccttcag ccagtgacga |
| 105781 | agaccttgac ccccatggtc tcctgggctg gacttctgat accaccaccc tgtgtgaatc |
| 105841 | tcccagggtg gcggtggaat gagctctacc ttcaagaatc aaagagccca gtcagagagg |
| 105901 | ttgctggcag ctctgcagcc ccagggcggt ggtgacaaga acacagcgag gtttattttt |
| 105961 | aaacctttgt aagtcccaca ctggtcacgt gtatagcacc ctcgcctctc caggtcattc |
| 106021 | tttggtaatg agtagcagtc aactcccaac caaggaaagg gcctgtcggg gtctttgggg |
| 106081 | acagagagga aggggcagca ggtgaggaat aagacctggc agcagcctgg ggtgcagggg |
| 106141 | aaatggggca gcccctgggg cctggcttgg aaaagtggcc atggggactt gatggtggaa |
| 106201 | aggccttgga tactctcaca cacacactca cactctgtgc acacacacac tccatgtaca |
| 106261 | cactcaccgg ggacttcaca gtggaaaggc ctgtacccta ggcctgcaac acacccactc |
| 106321 | agaaaacaca cccacttccc aatcaacacc acctgccaag ctaccacctc tgccacctcc |
| 106381 | tcagcttgtc tctctccaaa acaaattcca cttcctccct ccctcccacc aStctccctc |
| 106441 | cccaccccac ccctgcagcg ctgacagcca gcagccctcc tcccctcgcc ctcattactc |
| 106501 | agagttgacc ccaagaagga tctgtaggtg ctggcagtca ccccgggcac tgacaggcct |
| 106561 | cctgcctgcc cgcactgccg agggtggtca ccctcagctg ccctccccct ccacacccct |
| 106621 | ctgtgaagcc agggacagct tgacatgtag cctgcagccc tgtcacagcc gcctctgagc |
| 106681 | aggtgaaagc aagtcaagca gaatgacgag gcaacagcag actggggaag gggctctggg |
| 106741 | agactaaaaa tacctccatg gggaagggga ggagtcgagc agaactggat gtacgagggc |
| 106801 | ttctggggga gctgccagca ggaaagggag ggtgctgtgg gagccaggtt ttcatctcct |
| 106861 | acagcagggc tgaccagcag agtgactctg tgccacactc gctttgcctc agtttcctca |
| 106921 | attgcaaaat ggggatcgtg tctatagggc acttcagggg acatgcaagg gactcttcct |
| 106981 | gaggcagYag cactcttagg gtctcttaga agatctcagg tttggaaact cagaatccta |
| 107041 | cttggccttc tcaaatgtca cctcctcaga gaagcYctcc ctgaccaact tacatccact |
| 107101 | cacaccctct ccattgtttt ctttcccctc ccatttcatc atggcactga gcgctgtctg |
| 107161 | gcattagatt atacctgtgt tggtttacaa gttgattttg tctcccctac atggagagac |
| 107221 | aaacatggtg gagaccatgt ttattttggc ccccactagt ctcagaacct ggcacagggc |
| 107281 | cagccacata agaagcactt aataaacatt cgccaaatgg atgaatcaat caggcaggca |
| 107341 | gcgagtcagt catctctggt tcttctcctt cattctctgt atccaaatgt tattgcttct |
| 107401 | tcctccaaag gccatctagg ctttgcattt cctttccatt gcacacccta atccaggagc |
| 107461 | tcatcgactg acccctgagc tgttcaaaga acccactaag ggggatctac ctccacattg |
| 107521 | cccccctagt ccaccctaac gggggatcta cctccacact gcccccctag tccatcccaa |
| 107581 | gggactttca gaccattaga caaagggatg aaagacagga gcccatggtc ataggttcac |
| 107641 | gcattcacac gttcattcat tcattgccat ctacaaccca cttacacagc cagtggcaac |
| 107701 | ctagtgctga tcccctcggg ttcaccccat gattgtgctc tcctctgccc atgcacatct |
| 107761 | gatccaggct ttgcccttgc taacctccac tcctccaaga atcttcctct tgccacacta |
| 107821 | aggagtgaaa agagcatccg cttaaagtca gatagacaca ggttgaaaca ccagctatgt |
| 107881 | gtcttgggca agttacttct ctaactttta gttttctcaa ctgtaaaatg agcacaccaa |
| 107941 | cacctatata gcagagctgt tgagagagtt tgagctgaga gtgtgtgaaa Scacagggga |
| 108001 | cagcactaag tccacaggaa gcactcaaca catcatttcc cccctccttg cctaggctga |
| 108061 | tctctctcct ctgaactcct gttgcctttg gttagtgcac agtcaggtgc ctgctgtttc |
| 108121 | taatggtatc atgggtatta gcatcttctt cccagtttga ttcgaaggtc cttgagggca |
| 108181 | aaaatggcat tctatacttt attatgaccc cctaacactt Rgcacatagc gtgcaaagct |
| 108241 | cttttgcata tagtaactca gttaattcct acaataaccc tatgagttag accaagcagt |
| 108301 | gttatgattc ctagctggcc cttgatgaag caggttctca cctgtccaca gtcccagggc |
| 108361 | cagcagaagg agcaggacct aggtctccag gctctgttat agaaagcttK gaaggtgacc |
| 108421 | agccaaagga agccaatcct tcttccccaa gaactgccca ccctacttca ttcctctcta |
| 108481 | ccagtattcc cacactgctg ggccatggca gccatgagtg taccacatgg ccctgtccca |
| 108541 | ctggccacag ttgattggct ggctcgggag tagtcacctg attcaagctg ggccaatcag |
| 108601 | atcctacctc caaggggttt ggaattagaa aacagtgacc ctagctagta taggccactt |
| 108661 | gaactgaggg cccatacatt caggagcctt atggggccat gtacacatgg aagcaggaag |
| 108721 | aatgaaggag ggagaagtag aggccagaaa ccacctgggt tNctgtttcc caatgctaag |
| 108781 | tccctgccat gttcctgctc ttcctgtggt ttggatcttc aaaggttgct caaatttggg |
| 108841 | gcagtggcct ggcagctttt caaatccttc ccatttttat tgaagctgaa agacccttga |
| 108901 | ctaggacaga ctcggtccca agcWgagagc tagatcttct ataccagttg ttctcaaaca |
| 108961 | gtgtggccca ggaatcctgg gggatccctg agtgtccttt cagggactct gcagtgtctg |
| 109021 | aactattttc ataggagtcc taaaacgtga tctgccttgt tcactctcac gctctcacag |
| 109081 | gcatccagtg gagtcttcca gaggcgacat gacacgtgac gatgctgtca ctgtgtgtgt |
| 109141 | attctctagt ttaaaatttg ttttattttt cttgtaaatc ttttttactg ggctacctct |
| 109201 | taaaccacaa taggttcaga gagacaacct ttttaatttc taaaatggca aatatcatta |
| 109261 | gtggtatgaa tattactggt tataacctga ataacagcct ttgggggctc tcagtgtaag |
| 109321 | gaattcctga gatcaaaaaa attgagaacc actgctctac acccttgcta tttttttNtt |
| 109381 | ttttgagacg gagtcactct gtcatgcagg ctggagtgca gtggctcaat ctcggctcac |
| 109441 | tRcaacctcc gcctccgcct cccgggttca agtgattctc cgcctcagcc ccctgagtag |
| 109501 | ctgggattac aggtgcatgc cggcatgccc agctaatttt tcatattttt agtagagacg |
| 109561 | gggtttcaca ctgttagcca ggatggtctc aatctcctga cctcgtgatc cacccgccYt |
| 109621 | ggcctcccaa agtgctggga ttacaggtgt gagccaccgt gccaggcctt tttttttttt |
| 109681 | ttttttgaga tggggtctca ctctgtcact agggctggag tgcagtagca caatcacagc |
| 109741 | tcaatgaagc cttaaccact caggctccag tgatcctccc acctcagcct cccaagtagc |
| 109801 | tgtaaccaga ggtgcacacY gctctgcctg gctaattttt tcagagatga gttctcacca |
| 109861 | tgttgcccgc aggctggtct caaactcctg ggctcaaacc atcctcccac ctcggcttcc |
| 109921 | caaagtgctg ggattacagt tgtgagccac cgcacccagc ctacaccctt gctatttaaa |
| 109981 | gtgtggtcct gacaccagca gctccagcat cacttgggag cttattaaaa aggcaggagc |
| 110041 | tcagccccca ccccatggac ttactgcatc agaatttgta ttttaacaaa atcctcaggt |
| 110101 | gctccatatg cacataaagt ttgagaggca cctgaacacc acagtacata aataaatact |
| 110161 | tcatgattga ttgataattg attaattggt aatactcaca tggatgactc acaagaatct |
| 110221 | catgaatttt aaagagcttt gacttcttgg gctgaagtta ccaaagacac aattattaat |
| 110281 | tgttgggctc ccctcaccac tcagagaata cccattatca aaattccaag agttattccc |
| 110341 | tttgcttgcc ccctcctgat aatatctagg cagtatgaag cactcaaaac ttactgatga |
| 110401 | agaaactgag gcccagggta cttatacaat ttgcccacca tcacagaggt tacataattc |
| 110461 | atccaacgtg tagtaagtgg tagagctgga atttgaactt gcaaggtcta agttcaaata |
| 110521 | aagcagagcY tattctgtaa tccctacctt agccctggcc tcctcagtga gcacacaaag |
| 110581 | cacctggggg cttgtcaaag tgcagactct aattctcatt aatctagtgt ggagtctggg |
| 110641 | cttctgcatt cctaatgagc tcccagatga tattaatgcc accatccaga gaccacacac |
| 110701 | agaggtgtca agacttagac tgcctctagg ttctgacaag acaaatgggc atagttgttc |
| 110761 | tccagtgcca gcagcagctg gaatatacca ctttaaatgg tgggcaatga gctgggtatg |
| 110821 | gtggctcatg cctgtaatcc cagcactttg ggagacccgg acaagaggat cgcttgaggc |
| 110881 | caggagttcc agatcagcct gagcaacaga gtgaggcccc catctctaaa acataataat |
| 110941 | tataaaaaat aaagcWtggg caaaacagct gaactgaggc tgtgagtgca gcagcttcac |
| 111001 | acaccccagc acaggaggct tgcccacagc cttctgggtc cttccatggc tccgtgcatg |
| 111061 | ggtaacatgt ccacagtatc actgtacaac atggaaccat tcagctgctg cttggccagt |
| 111121 | atctggtggg cccagagcca gtctaggctc catccagccc cctcactgtg ttcaggtgtc |
| 111181 | tctcatctcc cctacagctg aaatacgact tttctttctc ttttccatca ttcaatcatt |
| 111241 | cattcgacaa gctaatcatt ggctatatgc tgggccctgg gccaggcatg aactcagagg |
| 111301 | agaaagcaca gacctgtgtt caaatcttag cctcttcctt actaggctga gtgaccttga |
| 111361 | acaaattgct taacccccct gggagcaata gtaccagctt gtggaaatga aagaagttct |
| 111421 | gaccaataga aatacaatgt gagccacact catcatttta aaatttccag cagcacattt |
| 111481 | tttttaaaaa aatagatagg tgcaattaat tttaataata tattttattt aacaatatat |
| 111541 | ccaaaatatg atagttttat aatttaatat aaaaattaag aatgagatgt tctgcattcc |
| 111601 | tttttcatac taagtcttca aaatttcata tttattttat atttacagca catttccatt |
| 111661 | aggactagac acattttgag gactcagtag ccacatgtgg ccagcagctg ccataatgca |
| 111721 | cagtgcagaa atagagatag gagtctaaag cacctggcac aaaggcatca ctctctatgt |
| 111781 | ggtagctctt gttatacaga gcccatcaca ttgcctggtt tacaggaagt acacagtaaa |
| 111841 | tatacatggt tgcactgact gctgatgttt tcacagacac ccaattcata tccactgact |
| 111901 | gcaagctcaa tcttctaaat gtaggttggg gagaggtgtg aagaggacca tttcccacac |
| 111961 | ttgcRtcagt ttggttgggg tgagagaagg aaagSaagtg ggtctggaca tgcacagctc |
| 112021 | tttgggagtt caccaaaggY ccaggaagtc caagctcRaa agtctccttt aataatggaa |
| 112081 | ttcaattttc tttttaatac cctcccctcc gtgaatgggt ctaggccagt ttatcaatgg |
| 112141 | ctgcaaaaat cattaagtaa aaacttgatg aggaactgta taagggagag atcaggctga |
| 112201 | caacactcaa atctactgat caattaaaac accacccaaa gcagaactgg acagggtatg |
| 112261 | cccctgatgt gatacgagaa gaacctatca ccacctagga aggattctta cctccccaca |
| 112321 | agaaatgttt gtacacacat acacaaacac acacacacac aagcacacac cttgaatcta |
| 112381 | agcaagtttc taatcaaact accagttttc aggaaacaca gaggatggag aaagacatta |
| 112441 | aataccaccc tcccaccact tctagaatgt gagaagttcc acaagacaaa caacccaaat |
| 112501 | tcttcaacaa attaatggca tggagaaaaa aaaatgtgtg tggtggggtg caatacagaa |
| 112561 | gagaatacag aagaggtatg ctgtggaatc cSggagactt cagtttcaaa caaaccaact |
| 112621 | ataaaatggc attttttgac aaaaggggaa atgataaggg actagatagt aggtattata |
| 112681 | actattattt tattggatat gataatgata tctgttaaag acacacactg agatatgtgt |
| 112741 | cagttaaatg ataagatgtc tagaacttgt tttcaaatac tccagaaaaa aaatcaaaca |
| 112801 | aacacaaatc aacaaaaagt aggagaaaag aagagacgag ccaagatagc caaaatcttg |
| 112861 | ctaagtgatg aggctggtcc cagctgtacc agaagtgcac catcagagag ctgcccgccc |
| 112921 | agcccctgca caggctgcac agccccacat gcgacaggct ggagcatctc agctcagtgt |
| 112981 | gtgccgcgct ggccgatgtg cagcggccgc ctgggaactt gcttctaata tagtataaag |
| 113041 | cccYgcccca gagctcctaa agcagaattt ctgagtgggg tcctgggaat ctgcattcaa |
| 113101 | taagctctct cagtgatcct aatgtctgct gaggcatgag aaccacaaga gtggagagcc |
| 113161 | ctgccatggt tatcattggc agcaaaccca cagaatgcct gggactaagt gcttatccgt |
| 113221 | gggaggggta agagtcccgc ctagagcctg aaggggtgag atYctcaagc ctgagagtcc |
| 113281 | attttccagg tggacagcag Yagtttactt ctggaggtcc ctctccacct ggttattgtc |
| 113341 | acatgccctt gcagacccca gacggccacc acagaaagcc ccttcgggcg tgaactctag |
| 113401 | attcaacctg aatgcctgga agtgcctgac cagtctcttg tttttattcc ttccacctgg |
| 113461 | gttttttccc ctctgccctt ccccaggctc tggctttttc cttgtcccat ccRaggaccc |
| 113521 | ctgggatggt ctcaagcctc cagagctcat gccaaaccca ctgggagcca ccagctgcag |
| 113581 | ccccctgcta ccccggcaaa cttctttttt tttttatagt catgattatg gtttatttNt |
| 113641 | tttttttatt atactttaag ttttagggta catgtgcaca atgtacgggt tagttacata |
| 113701 | tgcatacatg tgccatgttg gtgtgctgca cctattaact cgtaatttaa tattaggtat |
| 113761 | atctcctaat gctatccctc cccccagccc ctatctcaca acaggcccca gtgtgtgatg |
| 113821 | ttccccttcc tgtgaccatg taccccaaaa gcaggtaccc tgactctgtc attctggcac |
| 113881 | agcaccaggg acaaggcagg aaatgtccag ccttctttag taaccaagca gtggtgacaa |
| 113941 | caggcacatt ctccttatct tccaccctca ccctgatccc acccttcagc cctccccaga |
| 114001 | cctcaccacc cactcagggt accccctctg cctcctctcc tccctcttgc cagctctgaa |
| 114061 | ctcttccagg ctacgacctg ggatcagagc tSttctggag ttttctcctt tgagcgttct |
| 114121 | ccatgtaaga ccatggccat gatttccatc tagctgcagt ggagatcaat cgttggccgg |
| 114181 | gcaccccagc ctccctcatc tctttcctcg ctcatgtcat ccccaactta cccgtcctct |
| 114241 | agtcctgttc tttcgcttgg gaatatcctc ttccaaatcc tcttcttcat tcccttcttc |
| 114301 | tacattttca tcattttcca aaaccctctg aaatgaaaaa aaaaaataga aaaggtgaga |
| 114361 | aacgaaatgc aaagggaaaa accatcagag agtaatttta agggtgttct gatttgttgt |
| 114421 | tattttttaa cMaagtgaag acgctgcctt tgcttgggca gttgggccct ctgaaatgct |
| 114481 | gtctggagtt tgaactcttg gcactaccga ggctctccta agcccttggg ccccttccag |
| 114541 | acaagtcctc ccagaacact gacctttttc cattcttttt gcccaagaag agttttctaa |
| 114601 | caagagacta tgctggtagg ggcccttttc tatgtggggc caWaatgttt acttttgtaa |
| 114661 | atgtaatgtt tctttttgct cccaaagggc cacctcagaa atatgaaaat gtttctcctt |
| 114721 | tccctgccat tctggaaccc tgccctgtct gatgaaagca agtaacttcc tgttcatctg |
| 114781 | accagattta acacgaatgt gctgttcata ataagccact gctttttttt cttttttttt |
| 114841 | tttttaagaa aaatagactt tcatttagta cccgacttgt catggagctt aaagataatg |
| 114901 | aattttaaat tatctctctc cagcagcccg ccagtcacat tgcaacatag aatccaattt |
| 114961 | ccattttgca ggtgaatacR ctgaggctga aaacagtttt gagtgctcca gatcatccag |
| 115021 | tgaagccatg accaaagcaa ggtcaggttc atcggctcag ggttccccat tcatttctca |
| 115081 | gcacagtggc tgagaattcc agatgtaatc tggcatactc tccatgtagg ggcctaagca |
| 115141 | cgtcactccc tttagaggaa ggaaggaggt aggatgtgtt cctctgggct taagggaggg |
| 115201 | ataaaagact gtagcagagg acccagcccc acaatgggag taggaaaaga gggaaggggc |
| 115261 | atggagctca ctgggctcca tgtcaggggg cccagagggg tttttgtttg tNttNttttt |
| 115321 | tttctgagac agggactcac tttattgccc aggctggagt gcagtggcac aatcacRgct |
| 115381 | caatgcagcc tcaacctccc aggcctaagc gatcctccca cctcagcctg ccaagtagct |
| 115441 | gggactgcag gcacatgcca ccatgcctgg ctaatttatt ttttgtagag atgggggtct |
| 115501 | cactatgttg cccgggctga cctcgaacac ctggtctcaa gtgatcctcc caccccagcc |
| 115561 | tcccaaagtg ctaggattac aggcatacca tgcagatgag ctaccacacc tggccacctg |
| 115621 | ggggKttctt tatgggctca ctgtgtgatc tagggcaagt catcatctct tcatccttca |
| 115681 | gtttccccaa ttatacaata ggaattcatt cattccatct acaaacattt atctagtgcY |
| 115741 | gactacRtgg caggctccat gctaggcaag ctgcaaaatg gctacagtcc cagccccaaa |
| 115801 | tggactttcc agtctaggaa agggagaaca gatttaaaca atgtgcatat gtcaccatat |
| 115861 | aattacaact cttctcaaaa cagtggtagS caagttcaaa gtgctatgag agggtgttag |
| 115921 | gagggagttt tccctaatcc aagggccagg ggaggcctac atgagaacat gagctgagaa |
| 115981 | gttgagatgt ggaggatgag caaagactgg ccttgtgaat gaggacagag gggagggagg |
| 116041 | gaaaaggttc taggcagagg aaacagcata agcaaactcc ctgaggcagg gagaggaaca |
| 116101 | cggcttgtcc aaggagcaga aaggccagtg tgaccagtgc ttagtggctc aaggagagat |
| 116161 | ggcataagat ggagctagta aggggaacaa gtaccatact acacagggta accccatctc |
| 116221 | ctcttccttg tggtgatggg tcaaagatca caaatatgtt gagaaggctg agacagtggc |
| 116281 | acacactgca ctgttgtctg ataaccatcg ctgtttaagc cgcctcctgg atgacacctc |
| 116341 | tggcctgtcc tcaaagcagg aagcccgggg tgagccacca ggcagtgatc ttccaccact |
| 116401 | gcccctggct agaatgttct gctctaaatc acagggatca ggacacttct agtcacccaa |
| 116461 | tccatccaaa gaactcaaaa caggtctagt aaattcttct ttgccttttc tactaggttt |
| 116521 | atttgggaat ccaccagctg acagcactta aagggtctct gagccagcct taaccatcta |
| 116581 | acagagaaga agcattgtct gctggacatg acctctggcc cataactagg aaacgtcagc |
| 116641 | ccccattcac agtataagtc tcttgagttc ctgacctgta ggtgggaaca ctagcaaaag |
| 116701 | ccgactgaga cagtgacagg tcaaatgctc tgcagccttt ggggcaaaga catagtataa |
| 116761 | atataaggtc tggttatgac tgttgttgct gttcgtagaa ccatcttgca aactttccag |
| 116821 | gtcttgaagt cacttaatca tggtaagttc caagtgcctc gaatttaaag gtcatagcct |
| 116881 | ctggcatctc agtacaactc cgactctcat ttttcattat aagggtattt attacccgca |
| 116941 | atatggactc tgggttggaa ggtgaaagac tggtagaggc agctgagcta gtccccagag |
| 117001 | tgtcacatgg cctccatatc tgggagtaaa ctgacttctc ttctccctga ggctccatcc |
| 117061 | tccctctgcc catcagcttc cctgtgactc taaagcctct gtctatgccc tgggcctcag |
| 117121 | atgtgatggc ggcagaccca gtgtgtctgc atttcctgcg atgcggccct gacaccagaa |
| 117181 | gcctctcgac ataattcagg aggagccagg agtttctaac aaacaaagct tgtttcagac |
| 117241 | ctcaggaagc ggagggggag tcctgcatca ggagtcgcYg acgggggtgg ggtgtccaac |
| 117301 | tacaaagtca gctgaccccc cagtaacaga catgcattcc ttgtccaagg acattatctg |
| 117361 | gaaaataaac acattttaaa ataacttttc tccctgttac tctaaacagt ctttatggtg |
| 117421 | ataacagcgg tttgtggttg tgtttaaaaa aaaaaaatcc tttttaaaga ctacattatc |
| 117481 | tctggcctgg aatactgcaa aaatctcctg atttccctcc tKccattgct ggccccacat |
| 117541 | ccaatctcct ctccatgcag caggcaacag gatctttatg aaatacaaat tcacggccct |
| 117601 | gatcaaaatc ttccaaatgg cttccttctt tatatcacat ggaaccaaaa gccacttccc |
| 117661 | tgtcctgtgg gaactaggtg gtctggtctc ggcccacctg tcaaactcct gggctctttt |
| 117721 | ttccctggca caatactccg gccacacagg tcttctgttc tctcaacaaa accagctctg |
| 117781 | acctgtcttg gggccatcac acccactgct cctctgccca gaaagctctt ccccaggacc |
| 117841 | atcactttcc tgactcttct catcccatag ggttgaggtt aaatccctca actcgaccag |
| 117901 | atgcggtggc tcacgcctgt aatcccagca ctttgggagg ccgaggcagg cggatcacga |
| 117961 | ggttaggagt tcgagacgag cctggccaat gtggtgaaac cccgtttcta ctaaaaacac |
| 118021 | aaaaaatagt tgggcgtggt ggtgggcacc tgtaatccca gctactcaga aggctgaggc |
| 118081 | aggagaatcg tttgaacctg ggaggtggag gctgcagtca gctgagattg taccattgca |
| 118141 | ctccagcctg ggtgacgagg cgagactcca tctcaaaaaa aaaaaaaaaa tccctcatct |
| 118201 | tcctcaggaa ctctactctg atgctgaatc ttcatactgt ttgttctcat ggattttatc |
| 118261 | ctaaaatgca cttattttat ttgtattcct tgtttcctgc ctgtgtgtca cattcgaata |
| 118321 | tgagtagaga ggataggtat ctcttggtct ctgtgtccct agcacccagc acagtgcctg |
| 118381 | cacagaggaa gccctcatga aactttggtc aaatgaatga atgaatgaaa aagcataatc |
| 118441 | cattcgctac atgtgtattt taataatgca aagtcaataa aaacacgtaa actcatttga |
| 118501 | cttatggtcc tggctttgtc ttctagcaca gtagacagca gacccatgcg ctgcactcag |
| 118561 | cagaccttca gtttacatag gagatgatgg tgacatgatg acgacaatac agcaccagcc |
| 118621 | cagtgctgca tgagaagaac tcaagcccac caccagcttt aaagggaaag ggacccacat |
| 118681 | acccagagca tcaaggctct gggcagtggc tctgccccat Kcattgattc tgtcaaatta |
| 118741 | catgaactaa gcaaaaaaat actctagcaa gtaacacaga gatgacagac cttgacttca |
| 118801 | aaatctgaaa gtcacataca cacacacaag gaagaaagca gtaggtggct taaacaccaa |
| 118861 | aggcaagaag agatccctct ggctggaggg gagatcaaga aaggcttgat ggggaaggtg |
| 118921 | gcactggggt tgagtctaaa agatgagtga gatttgtaca catggagatg gaagaaaagg |
| 118981 | catgccagat aaagggcaca gcgggaccaa tggcatggag ggaagaaggc tgaagaacag |
| 119041 | agcggcgaaa ggagtagtgg aaaataagcc tgaaaactag gttaggaatg gagaggacac |
| 119101 | gcccaggaag gtcacgctca tccttttcga tttatttagt gggaaacaac agttttttag |
| 119161 | cagagttttg ctctagaaca aaaggacagc tagcaaagta ggacatgtca gagacgggaa |
| 119221 | gaactgggga cagggcgatt cctggaatct gttccctcat gtgtccattc aacaaatatt |
| 119281 | tatgttgcac ctggtaagca ccaagcactg tttgggtgct tatgagatat aagcaagcaa |
| 119341 | aactggcaac ggatcctgcc ctcgaggagt ttacattcta ctggaagaaa acagacaata |
| 119401 | aacaataaca taataagtaa gtaaattaga aagtacatta gatgatgata agagtgatgg |
| 119461 | aaaagaacaa gaaaagcaga gtggaatcgg gaggcagggg cagtcagggc aagggtgtga |
| 119521 | gatgaagtac ggcacggtca gggacatctt tgagagaaga ggaaggccag gcaccgtggc |
| 119581 | tcatgcctgt aatcccagca ctttgggagg ccgaagtggg tagatcacct gcagtcagga |
| 119641 | gttcgagacc agcctggcaa acatggtgaa tccgcgtctc tactaaaaat acaaaaatca |
| 119701 | gccagaggtg gtggtgcgca actataatcc cagctactca ggaggctgag acaggagaat |
| 119761 | cacttgaacc cagtaggcag aggttgcagc gagccgagat tgcaccactt cactccagcc |
| 119821 | tgggcgacac agcgagactc tgtctcaaaa ataaataaat tcataaataa taaaaaataa |
| 119881 | aaattaaaaa atgaagaagg tgagggtgag agacaagtgt acatcYgggg aaatggccag |
| 119941 | ggcaaaggcc tgaggtctgg gacaggcctg gcaWgttctg ggaggaacag aaggagattc |
| 120001 | ttgtgcctgg agtggagtgg gtgaggaaaa gaggaagagt ggagtcagag aggggtgaga |
| 120061 | aggccctgaa tttcactctc agatagcagg ggcggtagcg ggttctgagc acagaaatga |
| 120121 | taagaataga cttaaatttt aaaaagctcc ctctcctgtg gtgtatctag acaacagaat |
| 120181 | attattcagc attaaaaaga aatgagctat ctggccatga aaagacatga aggaacctta |
| 120241 | aatgaatgtt actaaatcag agaagccaat ctgaaaaggc Racatactgt gagtccaagt |
| 120301 | atatgacatc ctggaagagg caaaactatg gagacgggga aacggccagt ggttgccagg |
| 120361 | cattaggacg gagggaggga tgaacaggtg gagctcagag gatttttagg gcagtgaaac |
| 120421 | ttctgtatga taccgtaaga gtggacagat gccattacac atttgttcaa acccattgaa |
| 120481 | tgcRcaacgc caagagtgaa ccgtcatgca gactttgaac attgggtgat aatgatgagt |
| 120541 | cagtgcagct catctattat agcaaatgca ccactctagt ggaggatgtt gatcatgggg |
| 120601 | gaggctatac caggggtaca caggaaatct ctgtaccatc ctcttcattt tgctgtgaac |
| 120661 | ctaaaactgc tgttaaaaaa tatttttttt taaagactgc ccctcaagca ctatcaggga |
| 120721 | gaccactgag aagacaaccg cagcagttca cacaaaagat gagagcagct tgggccaggg |
| 120781 | tagcagtggt gggtgcgggg agaggggatg cgggagaact gggcttatgt tgaaggcaca |
| 120841 | gtcgacaagg ctcctgatga gcaggatgtc aggtgtgaga gacaagcagg agtcaaatat |
| 120901 | gaccccaagg tcccttagac aaataaaggt gacagttaat gaggatcttc actagggctg |
| 120961 | cagctcgtgg aaaagagagg agggaacaaa cacaagccat gttgtggcaa ctcaatggaa |
| 121021 | aagatttgac aattcatctt aacacgggaa tggcggagaa gaaggccaaa ttctgtgatt |
| 121081 | cattcaatgc ctttatcgag actcaactct acccagcttg ggaaaggccc cggggacact |
| 121141 | ggtgagcatg accgcattgg ccctggctat cgatggaact taccaagcca accagcaatg |
| 121201 | tattattcca ttattattaa tttaattgta gttttaataa attaatatat aaattaataa |
| 121261 | atttattgaa tattaaatat gcacaatagg tatagcataa aatataaaca tttaatatgt |
| 121321 | aatataaatt taatagaaat acaatttaat tttaataatt taatattaac taccaatgct |
| 121381 | attatttcat aataggatca attcatatta caatactcaa tggcaaacat tgtattagac |
| 121441 | aatgaggatt ccagacaaat aacaaataag agatggtagg tgtgaagtca ggtgcagtga |
| 121501 | ctcacgcctg taatcccagc actctgggag gctgaggcag gcgatcactt gaggtcagga |
| 121561 | gtttgagacc agcctggcca atatggtgaa accccatctc tactaaaaat acaaaaaaca |
| 121621 | gccagatggt ggcacatgcc tgtaatccca gctactcggt aggctgagac acaagaatca |
| 121681 | cttgaacccg ggaggcagag tttgcagtga gccgagatcg agatcatgcc actgcactcc |
| 121741 | agcctgggtg acagagcgag actctgtctc aaaaaaagaa aaagaaaaag aaaaagaaac |
| 121801 | ggtaggtgtg cacccttata atcctattca gtgagagttt tagaagtggc atggatagag |
| 121861 | ggtagtggac ccatggggac agagcagcca tccgcctgaa gggtgaggac ggcatgggct |
| 121921 | tgagggatgg ctgggcactg ctgggaaacg gagtgtgcag tcatggggac cggtgaggct |
| 121981 | gggtgtgagg gtggggggtg ggcaagccgg actcacgtgg ccgtagctgg agcagacgct |
| 122041 | cccacgtttc aagcctagac aatggactct tggttccatg gggacaagga ttcagactgt |
| 122101 | tttgtccact ctgtgtccct gggaccctgc acagtccctg tgttcactag gctcaacaaa |
| 122161 | ttcactttgc atagacagac agaagagagg gaagggaacc gaggtgaagg caataatgac |
| 122221 | tccttcggaa gataggttaa gtcagaaaag aatttgggag cttcctggcc accatagaaa |
| 122281 | caaggcagtc tgagactgag ccccggccct tgaggggagg atctggagcc aagccggtgc |
| 122341 | tggagttctg gaagcgctat ggtgacagga acactctgtg gggtccccag caggtgYggg |
| 122401 | aatacctgta tttcctggat gctttcctcc tccctggcat ccaccttctt ctcaaccccc |
| 122461 | tcgccacgga gcaaggcttc cagcgtggtg ctctctgagg tgaacccatc cttcttcagg |
| 122521 | ggaagctcca cttctgaaaa acaaagatag aaaggcaggt caggccacta gaagcaggca |
| 122581 | gggcaggagg gacagtcctg gaggacacgc agggcccagg gctcctgagg aggactcggg |
| 122641 | gcctgtgctc tctcctcctg gagggagagg aacactggag ccctgtccag cttttccatc |
| 122701 | tccatttcat aatccagaga cagattccga ccatgcccac acccccagag gaagctgagc |
| 122761 | cagagcagaa agggtgcggg gtRgatgtgg gctcacacgg ccatgcttct gcctctccag |
| 122821 | ccatgacatc ggacaaggca ggcgtcagga gcgtggagcc cttattgcat ccacaaagca |
| 122881 | ggggtaacaa tacccaaccc cacaggtggt tttgtaaggg gacatgtgtt catttgcggg |
| 122941 | aaagtgtttc aaaaactcaa gcaatgcaca atgctggcaa ccaagtatgc tcattccctc |
| 123001 | ctatcccagc tcttgtctgt caaccccaYg acctcctcat ggccatgctg gctgcccagc |
| 123061 | tctgctttcc tccatgaaaa gctagttcag cctcagaaag gttgttctct tcaacccagg |
| 123121 | gggaagtgag agcttcaaaa gccacgggaa gaggagcgac ggcgaatgcc tcacaagccc |
| 123181 | cggatgagca gagtggccac tgcacagcca cgccacgacc tttttttctg aaggtatcag |
| 123241 | gagtttgatc agctacctgg ggcttgagaa gagaagccct ggggttgcca gcagagaagt |
| 123301 | ggcaacccga gccagccctg cccagaagag acaccagaga gaagctgaac cccttcctcc |
| 123361 | aggtgtccca aactcctgac ctgacagagg gttggtgtgt ttctttcttt tctttctttc |
| 123421 | cttccctcct ttctttcctt tccctccctc cctcctttct ttctttccat tctctctttc |
| 123481 | tttctttctc tctcccttct ttctttctct ttctttcttt ctctctctct ctctYtcttt |
| 123541 | ctcttttaga gacagggtct tactttatca cccaggctgg agtgcagtgg cactatcata |
| 123601 | gctcactgca gcctcaaact tctggcctca agcaatccat ctgcctcagc ctcccaaagc |
| 123661 | actggggtta taggcatgag ccaccatccc ctgctgtttt tttaattaaa gatgcaaacc |
| 123721 | taaggtacag gagatcattc tgatttacta aaatatttct tcaacctctc ctctgatcct |
| 123781 | ggacctcata cccccacact gccacagttt gatatgtggt ccttaccatt gcaccaggta |
| 123841 | agctgggggc aggaactatg atgcagtggt gtcccagaaa acaggagaga tgaatggcct |
| 123901 | gctcttttgg gggtattgag agtttagtgt agggactatt tacagagatg gggagaggtg |
| 123961 | attaagggat ctgacaaggt aggttaaagc atcccaggtg ggttttgggc aggggtgagg |
| 124021 | gtggagaggg gccattaccg ctgctgctat ttttggaggg acagagggag agtgcttgca |
| 124081 | agaacctgga gatagcacca ttagcaSagc cccaggacag gagctatggc ttgcagaagc |
| 124141 | caggaaccaa gggaagaaac aatccaacca ccctctccat ccatcctcca tcagcctgct |
| 124201 | gcgtcctctc ccaagggcca accttaaaca gaagcgagag gggaagggag cccccttcac |
| 124261 | acagcctacc gagctccgag cagggtgaag acggaaaagc atggatctgg aggggcaaat |
| 124321 | ggaaagagca gcaccaccga gcctgctcca ctgggctctg tcctgactcc aacagaatcc |
| 124381 | tacctgcact ttccacttcc tccaccagaa acagagggag tctgacYaat ccatgaggaa |
| 124441 | gcggcaagcg ggcatccatt aaaactgaac aaatgcacac ctgtgggtga ccggccccca |
| 124501 | gaaaaagaag gtgggaagga gaaacagcat ctaaaatctc ttgagaaaca aagtctttgg |
| 124561 | gatttttcct accaaaaaag gagagcaaag gatccacttc ccatcaagga gcgctaccac |
| 124621 | gaaatcagcc acctctgaat ccatctatcc attcaacaga tatttactga gcacctgcca |
| 124681 | taggaccacc actgtgctgg gactagggac ataggaggtg aaccaaataa agacaggYcc |
| 124741 | tgcctttagg aggcttacag tccaaagaga cagatagtaa atgcaaaata caaaatcaaa |
| 124801 | atgcaattac gaattgtcgt aaatgttagg gaggaaaacg acagaatgaa atgtgaaaga |
| 124861 | atataggtaa cggggtctac cttagatcag ggagtcaggg aaggcctgta tgatgaagtt |
| 124921 | acatttaagg agagtgtcag acaaacagcc aactgtctgc tttaatgagt tcatggagta |
| 124981 | catctccata gcctgctctg atgacatatt actctttctc ccaagtcaga aaagtcttcc |
| 125041 | aaatgtctac tctaaagcaa ttagtgtgtg ctgtctcatg tcctgtcctc agtcaaaaga |
| 125101 | gaacagctgg tcacaacccc tcaaacttat aataatcata tatacataga gttgaagttc |
| 125161 | atattttagt atccccctta gccttcgcag cactaaataa atcctattta ttagtgttat |
| 125221 | ttcccagaat ggtacagcat tttagagcta gaaaagacct tatagatcat ctaattcaat |
| 125281 | catctcattt tgtagatgcg tcacccgaga ccctgaaaag ttaagtaaMc accaacagcc |
| 125341 | acacagcagg atgagaatgc agctgaatga tatttccaag ttgccactga aatcacgaca |
| 125401 | atggccccct ctcattgtat tcttattagt ggttgacagg cttaggctga gccctgaatg |
| 125461 | agctttggta catccgtgta catgcagagg gcttggcaag cagcaaacat tcaaaactta |
| 125521 | gcagctgtta ctgctacgta tcaatcccca ctacacccaa gagcacagtt attaacccgt |
| 125581 | tttacagatg agggcagtgc agcttataga ggttgagaac ttgcttgata tcacacagct |
| 125641 | ggtaaggggc aaagcccggc cttaaactca atctgccttc agacccagtc acctaattag |
| 125701 | gctgcatgga ctccaccctg tctctctgtg gtccttcctt gaaaagtctt tttccaaacc |
| 125761 | tttatgcatt ttcattacat tctgagactc actccRtagt tttcatattt caggataaaa |
| 125821 | ggcagatctt taactgagga tgagggcagg tttgtgccat ggttttgggt tctgctctat |
| 125881 | acgatcatcc ccgtgcaatg ttcaccattc tgcgacagta tctacccact atagaatgtt |
| 125941 | ccatcaatag tgaacttgcc cctctcttcc ttctcctgct cttccttcct cctctgataa |
| 126001 | atatgcctcc atcctgcttt cccataattg aatctactct cttggtcctc tttaaccaaa |
| 126061 | ctttaccaga taggcacagt agaagaatca agagggaaaa cagaaccaaa tgggaaaagt |
| 126121 | gaaatggaaa aagcaagaag ggatctccaa tagcatcaag agtccaatcc cttaaacagg |
| 126181 | aaagtgaagc acagaaagat atagagattc ccccagggtc acagagacat gtagtggcat |
| 126241 | cttgcttccc taattaccac aataRttgat ttgctcccat aggttctgcc ttcagacagc |
| 126301 | cctggagtca atccctagcc ttgccacgta ttagtaaagg gaccatgggc tagctacgta |
| 126361 | acctaagcct ccatttccat atgtggtaaa tgggtgtggt catagtatct atttcagtgt |
| 126421 | tggcgaatca aacatggcaa tgtgctacat aaagaaacgc taaaagaatc cacacacaaa |
| 126481 | aaaactagtg tgataaatag ggtagaagat gaggcttatc aacttttgtt ttattctatt |
| 126541 | caaaacttag attcaaaatt agatcatata cgtaaagaat ctacaacatg gtaagtatcc |
| 126601 | taaaaatgct agatgttatt ccctaagttt gaatagtttt gatttcaatg gtacagctgc |
| 126661 | actgtccaac agaaatgtaa taccggccac ttatgtaatt tttaacttac tagtagtcac |
| 126721 | atgtagaaaa aggaagaaag tcaggtgtca ttaattttaa taagataWtg tatccgatct |
| 126781 | ggtatttcta aaacattacc attcaacatg aaatcaacat aaaaatatgg acgcaaaatt |
| 126841 | ttacattctt ttatctgcat tgtcttcaaa gtctggtggg tattttccat ttagagcgta |
| 126901 | tctcaattca gactagccac atttcaagag ctccatagcc acaggtggct accagctacc |
| 126961 | ataatggaca gtgcagattt gcagcattaa aaataacatt aactatgact ttgcttccaa |
| 127021 | aaaatggaaa gaattttact gtggttctta atgttgtcca cgtaatagct gccgagtgcc |
| 127081 | attccaagag cagagttcta gcggatcact gcaaaccctg gttgcaaagc atgatgggta |
| 127141 | aggcctgcaa ggtcagagtg aaagggtagc catccctccc tacaaacaaa cacagggtgc |
| 127201 | actctgcctc ctgaccacag gaggtcccag aatgaaggag gaagaaaggt aacaggtcca |
| 127261 | agcccttgca caatgtatgt cctaaacaca cttctattga gccctYaggg atttcattta |
| 127321 | tccaatttta tttgaatatg gcccatctgg atataggatt taataattct ttgaaagtgt |
| 127381 | actcattttg aacctcacac aaacttgggg cccacataac ccgacaggag ccattgaacc |
| 127441 | tttaacatta aaagaaaatg gtccaaaaca cacacataat tcatctttaa attagcctga |
| 127501 | ttttatacac atggatataa acccatggat cgttactctc tccRgagcgg cagcagctgt |
| 127561 | cacccaagcc agccgcctcc ctgggccccc gtaagcagct ctgcctctac ttctaattgg |
| 127621 | gaacagagat actccttggg tgacttccag cacgatccat cacaccagag acagcttctg |
| 127681 | tgcaaaccga agcaaatcgg tttctaaata agaaaaaacc tccgagacaa acaaggattg |
| 127741 | aagcatagaa agtcttttaa aaacaaccaa aaaaagcaca aataaaagca aaaaaacaaa |
| 127801 | caaacaaaMa aaaaaaNcca cacacacaca cagaaatttc cagcaaagag caatggcttc |
| 127861 | ttctctttag ggctacagac agggcagcca tcctcttggg tgcagatcaa agccacaagc |
| 127921 | ttttgacaca ggtgccccaa gcctgccacc caatgcactg aaagcaaagc ttccactggg |
| 127981 | ttgttggagc caccgagctc attaacagga tgcaggcgag atgagtctga aagtgcaaca |
| 128041 | tgggagcaca gtgagtaatt aaatcccagg tcatttaaaa atgcaaacgc cacgaatgtc |
| 128101 | atgagctccc caagcctctg actccttctc cttcgttatt tggctcagga tgaaggactg |
| 128161 | gaaacctgca agtcgtatat ggattataat aaaatatgcc atatagaaaa cagattaaga |
| 128221 | cagttcttct ttggggaaat atacaaggag aggagctgtc tgttagctgt cgagtttgca |
| 128281 | agagatacca tctttcggga ggcagtgtgt tctgccaaca gacagactgc cccaagggaa |
| 128341 | attgtggtga aacaccccga caaggcgcat ccatccgtcc atccatcatg ggctccctct |
| 128401 | gatgcagggg cctggagata gagttatggt gcaaaaggga gggagaaatc ccacagacaa |
| 128461 | tggcaaagaa tcctcctgct agatcatgag tgctcaattt tttttttttt tagctgcctc |
| 128521 | tagtttttct tcaaaaatac aaacatgcag ttttctcgtc tgggtgctta ttatattgag |
| 128581 | gagttaagct catcatattt ccttggattt atacatcaaa ttacacgaag gagaaaagag |
| 128641 | cttaagaatc attatttcat ttatccccat cagactgtca gactcgtgag gagggagaag |
| 128701 | ggagggaggt attgttatct ctcacctcac aggggaagag actggggagg gggatcagag |
| 128761 | acctgatttc ctaagatggt ggcaggccca gggatccatc tgctcattcc ctaggggtgc |
| 128821 | cactttcttg gctgcctttg actccctgtt ttgtgactca taaagctcga aatagatgag |
| 128881 | gttcacattg aggacccatc acccaggctg caccctgcag gccctattac cacttgcttt |
| 128941 | agaatctcag gatatccata taacaggagg actcatccct cccccatatg gcctctgctg |
| 129001 | cacctaccct ctcaccccta agtagacacg ccaggatggg ttacggcatc cagatatcag |
| 129061 | gaccacagat aagcattccc ccaccccgag atacagaaaa acacccacac tgcccaagga |
| 129121 | agctcaccac tgagctgcca gcagaccagg taaatgcagc taccaggagg gtgagacggg |
| 129181 | gagggtggtg ggagtggaca gagtgcgggc agcatttcca aaggcaaggt cggggaatca |
| 129241 | ggagaggctg gtcagatggt acagagtgtc agtcagacag gatgaatatg ttctggagat |
| 129301 | ctgttgtaca gcatggtgac tccagttatt agtaatgaaa attgctaaga gagtcaatct |
| 129361 | taaatgttct catgcacaca caaaaaatga caagaatgtg gagtgataga tacattaact |
| 129421 | agcttcattt agtcatttca caatgtatat atatatttaa acatcacact gtatgccata |
| 129481 | aatatatata acttttattt gtcaattatg ccttaataaa gctcggaggg aataaaaaag |
| 129541 | taaataataa aaatgcaggc tgggagcagt ggttcacacc tataatccca atattttggg |
| 129601 | aggcgaggtg ggtggataac ttgaggtcag gagtttgaga ccagcctggc ctacatggtg |
| 129661 | aaatcccatt tctaccaaaa aaacacaaaa attagccatg tgggatcgta cgcacctgta |
| 129721 | gtcccagtga ctcgagaggc tgaggtggga gaatcgcttg aacctggaag Sgggaggttg |
| 129781 | cagtgagcca agatcatgcc actgcgctcc agcctaggtg acagagtgag actctgtctc |
| 129841 | aaaaaaaaaa aaaatgcata gtgcaaagaa atacagacaa ccagaaagtg caaaaagctg |
| 129901 | ctctggggtg aactctcagc tccattaccc taaagcacac acttggcctt gatctcctta |
| 129961 | tctgtaaacc tgggagaaca aaacccactt cccactgttg ttatggggcc aaaggagata |
| 130021 | atacatatga agtgtctggc acaggtgaga tgctcaataa Statgagctc ccttctttcc |
| 130081 | acctcctaca gtttccctct ccttgtgctt ttcagcaaaa aaaccctcac ctttctgaca |
| 130141 | ctctaagacc accagatgtg gtgagtgagg cacagctaac cccaccatgg gcatgagccc |
| 130201 | agggaggatg gaaacaaacg gaatctgagg caggtaacag gatggaagag gccaagaatg |
| 130261 | agacgcagct ggaaagcaga cgctggtgct gctccaagtc cagggtggcc attgctcctg |
| 130321 | gagcttcggg ggRaaaatca tggcagagcc acagtggaag ataccctgct ttatcccttc |
| 130381 | atttggcaag taattatcaa gcacctacta tgtatcgact ctgttctagg tgctggggca |
| 130441 | atgactctga agaaaagaga caaaaatccc ggccctcata gaatttatat tctagcaaga |
| 130501 | gagaaacaat acatgaagca aacaagtgaa accttcagtg tgacagatgg taatcagtgc |
| 130561 | tttggaggaa aaacagagcc aaggaggaga agttggagtg tcagggtgat gggagggggg |
| 130621 | ttacactttt aaatgtggcc aggaaagtgc aagcctgaag gaagagaggg agggggctac |
| 130681 | gctgacatcc aggcaaagac gttccaggca aaggggcctt cagcacaaaa gctctgaagc |
| 130741 | cggcccatct gcctggagca cggtgagcca gggggatggt aggagaccag cttggggagc |
| 130801 | tacaggggca ttgaaggcca tgtaggcacc tgcaagaatg ttggcgtcca ccccaaatgc |
| 130861 | aatggcaagc cgctggaggt gttgagcaaa gcatgatata atgatgccta catgttaaca |
| 130921 | gaatcactct gagaataagt gaagggggtc ggagcagatg tggggtgacc agtgaggagt |
| 130981 | tactgcaaaa tgtatgtcgt ctcttaatga gtttggtgct tggggaggga gctatgcacg |
| 131041 | atatggacag agctctgtcc tcatggtact aatattgtgg ttctcagatg tgtcctgggg |
| 131101 | gcagtaagca ctggggagca gtgccctgag cagggggctt ccccaccagt gaaccaggat |
| 131161 | ggaggccaag tggagaggct ggtgtcaatg caaagtgggc aaaggtgtga agccacacca |
| 131221 | gggtgagtcc acggaggaat ggctcttcaa gaagaaagtg gaccagccca ggccaagggc |
| 131281 | agggaaaaag atctagccac atgggcctgg cctggcatac catggtttaa caggtgtagt |
| 131341 | aaaagcagag aggaggcctt cccagttcat ctctgtgccc tgggccatgg gtgcctcggg |
| 131401 | gctgagttct gggacatagg agtgaggtcc tgcaagctgt ggttctggta aaggtgggtc |
| 131461 | ccacggccaa gttctaggtc atctatagac tggggccttt taaaaggcta taagacagag |
| 131521 | ctgggcatgg tggcgcatgc ctgaattccc agctactcag gaggctgagg caggaggagg |
| 131581 | atcacgtgaa cacaggagtt caaatatagc ctaaggaaca cagcaaggcc cgtctctaaa |
| 131641 | aataaataaa taaataaaat aaaatgaata gactctaagg cagaatgagt tgccattctg |
| 131701 | ggccagatgg cacacacttt tccagcgggg cagYgagctg tctgcagcaa attcattttc |
| 131761 | ggtgttgggg acgggaactg tgttgcRgag acccacccag cagtgcagtg tggaggtaga |
| 131821 | atcacagtgc ctcctcacac ttgccaagtc ctccaccctc catcccacac gcctctcaga |
| 131881 | ataaccccgc aaaatcacca tgacccacat ctcacagatg aaggactgag gcaggagtgg |
| 131941 | ttaggaaatt gtccaaaatc acacagccag tggctggagg gatctagact tgaacctagg |
| 132001 | cctgtgatcc ctgtttggtg cctgtcctcc tgcagagcaa ggatatttgg acagggaagg |
| 132061 | cccctacaac ccaaaaccaa acagccaaga tggcaacact tgggcccccc aggccaggct |
| 132121 | gggaaccaca agggaagcca agctcagcca ccccaggcct ctcctctctg gcggagcctc |
| 132181 | cctcatctgt gaagcagaga tgatgagagc taagtcatgt tctccaaggt taagatcaaa |
| 132241 | caccagacat gtctctttcc ctcatccctt tagaaatctt tactgcccat gtactatgtg |
| 132301 | ccaagcctca tgctgggtgc tagggataca gatgaagaaa cagatgggcc ctcgccgtca |
| 132361 | agttgtccac ctcccagtgg gcagaggtac aggtgagcag gcaatggcat aactgggtga |
| 132421 | tccctcagac tcacaactca tgctctacac gtggctctgc caggtcccac tgcaaaacat |
| 132481 | gaccgtggac actcagcagc agccgtgcca ctgtcctgcc cactgcctgc tgccagccca |
| 132541 | cctcctcggc cagcccaagc tcccagagcc ctgcagaaca ccagcagagc tggggaacac |
| 132601 | gagcagagct gaagtcacac agacttcgtc ccYcagtccc tgctcctttc ctgagacgga |
| 132661 | caggaaggca aaaggagcag gtcccttcca gcacgtaata gccacagcct gagtgggggc |
| 132721 | tgcacgccct gcccacctcg ctccagctcc ccttctcatg aggccaacca gagctgacga |
| 132781 | agctgccggt gcaggcctcc agatggatgg ctgcaggctc tgacactgca ggaaagagat |
| 132841 | aaatgactgg tcatttcccc ctgatgcact tgaaggcatc cattctccca gccgtaccaa |
| 132901 | gctcaggcaa cccccaccgc ccagagacag ataagcactg acagatgggg gtgggagccg |
| 132961 | gagcgtcctt ccccctcgga aggacacagc cagactcctc tgcaggccgg gagaaggtcg |
| 133021 | cgttgtccat tggctcacac ctggctcgag gctccgcggg tcccttcctc catctccccg |
| 133081 | ttcttgccca cccccgcctg gctcatccat ctgaagagca cacggcagag ggggaaggct |
| 133141 | gggggcagaa ggagccgctg acaggctcat caatctcagg ttacatgctt ggcacctcgg |
| 133201 | cgttccttaa gttgttcccg gattgggagg gtgtcgcagg agccaggggc actgtcttga |
| 133261 | ggagaagaga aatcgcaccg aacatcagag agcaaatggt ggagaagcct ggagactatt |
| 133321 | aaaatgtggg ccatgtacaa aagcagtagg gaactaatta agatgcagga ttttcaaaag |
| 133381 | ctggggaagt tggccgcagc ctgctcccat ctgtagagtt ctggcagttc agcgcagcag |
| 133441 | ggacagcacc ctgggtgggt gaagtaggcc tcaccacccc tgctcgagct ggccagactc |
| 133501 | aagtccctcc gcctggacca cccctttggg agatacccct ctgcaggtcc ctacgacttg |
| 133561 | cttgttggcc agcctgtggc cctctctgct ttgggacacc ttaagccact gtcgtcttcg |
| 133621 | gggtgcatgc cctcttgtct gagtttcccc caacgacatg ggatgggggc gatcatcctg |
| 133681 | gtcgttcatc cccagtcatt gaaggactgc caaaaacaga aaaaatacaa gagaaagaac |
| 133741 | actatccctg gctgccctcc ctcccatcac cctcggaatt cggcaagggt ggcaaagagc |
| 133801 | caccctgctc cccaagagtc ctggagcaga agggatcgac aaggcctcag gtgcattcca |
| 133861 | ggtacaggag gagcgtcaac tgtaaagccc ttttcatctg cgggtccagg ataagtgagt |
| 133921 | gtgtcgggcc ttcaagcaac atggcagcag ctccagcagg ctccccatag ggagcatcac |
| 133981 | caggatcctg gcatcaccag gatcccctgg gaaattagct tcctaattct gtgccgcagt |
| 134041 | ttcccaacct ctaaaatgaa gataatatct gtcctagacc atggggaccg aaggtctggg |
| 134101 | gttgaaagat gctaaattga tctaagcaga ctatctgggc atttacttca aggggacaca |
| 134161 | agtaaaagtt tgttcccagc ctttgtcttt tcttcattca aaaaaaaaaa aaaaaggaaa |
| 134221 | agaaaaagaa agaaagtgaa gttaaaggcc tcttttttaa tcagctggct tttgcccaac |
| 134281 | atctgctgtg ctcccaggcc ttctggtgga tatttactct ccacctttct cttctgccag |
| 134341 | ctccactccc ccttcacaag gacagagccc ttttcctggg gagatggtcc gggggaggcc |
| 134401 | ccttgctggc accaggcccc tgcccaagtc tttgtgttgt agaattgtga tgtctacatc |
| 134461 | atgaaacacc ttgcattcgc ctctgtggcc aagtgcaagc tgcagagaac actgaactca |
| 134521 | attgtcagtc actggggaga aataatgttg tgtgtgttgt ccttcagatg cccaggcaag |
| 134581 | taggaggttg caagatgttt attcaagact gactagaggg gtgacatttg gcaggagaaa |
| 134641 | cacataggtc cagggacact gagaacactt gcaaattcca aagagttctt cccagatctc |
| 134701 | aggcccttcc tatccccatg cagacagatt tttattttca gcaaataata aagaaacaga |
| 134761 | agagggatgg tgcatttacc acactgaagg ggtgaggaca gcaggactgg gagtcactcc |
| 134821 | aggagggaat aatttcaccc aggaacaaat gactcgatgt cagccaagac ccagctcccc |
| 134881 | actttttttg agccagggcc tccctctgtc acccaggctg gagggcagtg gtgcaatcat |
| 134941 | ggctcactgc agccacaacc tcgcggactt cattgatcct cctacctcag cctccctagt |
| 135001 | agtgtcaccc tacaggtgcc caccaccaca cctggctaat ttttgtattt tttgtagaga |
| 135061 | caggttcttg ccatgtcacc caggcccagc tccccacttt ccagcctgaa aggtccactc |
| 135121 | atcccacatg agcaaagtgc ttcgggggac tgaatcagaa aaggatggtt ctgtggcact |
| 135181 | gtcctcagat cttctccaac agccctcaag attcttgaac gtgcaccccc cactctttgc |
| 135241 | cctgacctgg tttcctgaca cagctccgca gacctgagSt cagagaaaac tctgaccctc |
| 135301 | gttacccctg gacttcgttg ctgaaacctg cccatcactg ctggactcag gcagggacag |
| 135361 | aaaggaatac aggagggaca agctgaatgt tggcataaat tttttgtgac atctcctcac |
| 135421 | tgtccatagg agagtctgac gtggaaagaa actcatgcct tgagacaatt ctgctccagg |
| 135481 | ccagtggccc ctcagtctct tgaagagcag gcagactatt ctccctcttc ccggaggaga |
| 135541 | tgaggacatg tcagggtgtg ctgacggcag aacccaggtg ggcttcaaca gtactaggca |
| 135601 | aaactagatt cctgagcatc ctctgtcaag gggaggtcac accaggaggc tgggagggaa |
| 135661 | gatgaggaaa agacaggcat cctcaggggg tgggtgtgga ggggcaccWt gggtgggtgg |
| 135721 | gttggttggt tgggttcttt ccaacataac tcagccttcc agtttccata aatttaaaga |
| 135781 | gatgcaaccc aggatggaca aattatcgtt cttcagcaag ttttgcccag aggattatat |
| 135841 | acaatgttgt acaatttgct ttttgttgtg caatttgttt tttttcgcca cttagcaata |
| 135901 | tatctttaac atctctccat atttttttta ctcattcttt taaaggacaa aataatattc |
| 135961 | cattcataga atatacaagt actatatatt taactagtcc tcatcaatgg atcttctgga |
| 136021 | agtttccatt ctttgttgtt attacaaacc atgccacaat aaaaaatata tacccaatat |
| 136081 | atattatatg catatattca tttatagaat aagttcctag caatagaatg tctaggtcaa |
| 136141 | agagcatgtg tgttttaaat actggtaggt actattcatt gtacctcaaa aaggtcatac |
| 136201 | caattccRtt gcaatgtgta aaaaagagcc tctccctcca tctccatctc tgtttccctc |
| 136261 | acctgctccc ctctccttgg gaaggtcagc ctcagtccac ccagctaatg atcctcacct |
| 136321 | cctaggaatt tgcagcaaat tctccagaga ctctggccaa tgactctatc ctgagtcacg |
| 136381 | cgcgaagtat cagccaaaaa actgagctct ccttgagccc aaatcaaatg gaataaaaat |
| 136441 | aagagatagt tattttaaaa tactgtccga gttagccctg ttaaccctcc tgagggagga |
| 136501 | cataccccca aaaaagggag gagtcctctc aagtggaaac tgcattttat gaatgatgag |
| 136561 | ggcagagcct ctggaagttg ggcaggttgt gccctcaaag ggacccccac caggtgcaca |
| 136621 | gaagaggggc tgagatctag ctcgtgctct actaacccac cagaaagggc atcatacgtt |
| 136681 | ttgtaaacgt ccacccccca gggggcacca ccctccactt ttgcacaaac aaaaccatat |
| 136741 | gtgctagtca aggccctgga tgaagtcact cttctgaaat tttggcctgt gcaaaaaatt |
| 136801 | caaatgcaaa cttgcatcac ctctgggaag acagtgggta catttctttg gctgcgctgt |
| 136861 | ttctcattct ctagatgggc cctggctgct tggaggtacc aggctgaggc tcgttaccac |
| 136921 | tgactccagg gcactccgac ggcctcttca ttcaccaccc gcagctgcag gctggcatgg |
| 136981 | tggtacattc tgggagccag gtgcagcccc ggctgttcag actcagaaga ggattgggaa |
| 137041 | atgacaagca tctgccctag ggtttgtggg ggttgtttct gtgtttttag Yaggcctagt |
| 137101 | agtaggtctg tttacccaaa gaggggaaca agttcctctg agaaggtgct ctgccatgtg |
| 137161 | acagtcacgc attccagccc cggctgttca ttgcttctca gcatctgggg gctggtgtgg |
| 137221 | aggaataagc ctgccttggg ggaacccaac ttttcccccg agggcggata attgcaaagg |
| 137281 | ccagcacctg tctctgcaga tggggtcctt ccccgggacc ctgcccatcc ctggggtgga |
| 137341 | ctttcaccac tggaaacctg gtctagccac ttctgcctca tgcttgggca gaggctgaag |
| 137401 | aagcctctca aatgatcaca gatacaagac gccatgtgca ggtggggact tggctatggc |
| 137461 | aaaccgctag ccctaggttc ttcctttccc ttcccctcRc atgttacggc aggcccgaca |
| 137521 | acggggcatg gccaaagcag gctgtgaact cagctgctcc acgttcaccc acaggccagg |
| 137581 | cataaacact ggcaggcccg tgtgcccaaa gtaaatgttt ttaggcagaa ggcacaaaca |
| 137641 | ctcaagaaat agctgcctgc ctcagtccca ccaacatggg ctcctcagat acatgagcag |
| 137701 | cacaRcaaat tcttcttggc tctgtMcaca gtctgatttc tctcacctct aaaatgggct |
| 137761 | cacaaccctc ttacctcttc cttccctctc taccttccag ggctatggta gggaccaagg |
| 137821 | agctgacatg aaagaaaatg ctgcatgaac tacagcatgc tcttcggact ctggttatga |
| 137881 | tgatgatgat taaccagagt gtcccatccc ctgggtggca gcaattggtt gtgggggggt |
| 137941 | aatRacccca gccagctaat ctgtccttcc taggtcttct ctgccacagc tgttgagggt |
| 138001 | gactgccttt ctgctgggtg tggtatgtgc tgagactgga gctgctggca gccccctcac |
| 138061 | ttaccctatg aggagaagag agtgttaaag gtacagagag actggctggg tgtgatggct |
| 138121 | gatccctata atcccagcat tttgggaggc ccgaggcagg aggattgctt gaggMcagga |
| 138181 | gtttgcaacc aggctgggca acatagtgag acccccgtct ctaNaaaaaa aaaaaaaaaa |
| 138241 | atagctaggt gatatggttt ggctctgtgt ccccatccaa atctcatctt gtagctccca |
| 138301 | taattcccac gtgggaggga cccagtggga gatgatcaaa tcatggggga gggtctttcc |
| 138361 | catgctgttc ttatgatagt gaatgggtct cacgagatct gatggtttta aaaacaagaa |
| 138421 | tttctctgca caagctctct ctttgactcc tgccatccat gtaagatgtg gcttgctcct |
| 138481 | ctttgtctcc ccagccatgt ggaactgtaa gtccaataaa ccagtttctt ttgtaaattg |
| 138541 | cccagtctca agtatgtgtt tatcagcagt gtgaaaacag actaatgcag taaattggta |
| 138601 | ccagtagagt ggggcattgc tgaaaagata cccgaaaata tggaagcaac tttgcaactg |
| 138661 | ggtaacaggc agaggttgga aaagtttgga cggctcagaa gaagacagcg aaatgtggga |
| 138721 | aagtttggaa cttcctagag acttgttgaa tgactttgcc ccaaatgctg atagtgatat |
| 138781 | ggacaataaa gtccaggtta gggtggtccc agatggagat gaaaaatttg ttgggaacta |
| 138841 | gagcaaaggt gactcttgtt acattttacc aaagagaatg ggggcatttt gcccctgccc |
| 138901 | tagagatttg tggaatgttg aacttgagag agatgattta gggtatctgg tggaaaaaat |
| 138961 | ttctaagcag caaagcattc aagaagtgac ttgggtgctg ttaaaggcat tcagttttat |
| 139021 | aagggaagca gagcataaca gtttggaaaa tttgcagcct gacaatgaaa tagaaaaaaa |
| 139081 | aaatccattt tctgaggaga aattcaagct ggctacagaa atttgcataa gtaatgagga |
| 139141 | gcccaatatt aatccccaag acaaagggga aaatgtctcc agggcatgtc aagggtcttc |
| 139201 | acggcagccc ctcccatcac aggcctggag gtttaggaga aagaaatggt tgtgtgagcc |
| 139261 | aggctcaggg tccttctgtt atgtgcagcc tagggacttg gtgtcctgtg tcccagctRc |
| 139321 | tccagctgtg gctgaaaggg gccaacatag agctcaggct gtggcttcag agggtgcaag |
| 139381 | cctcaagcct tgacaaattc catgtggtRt tgagcctgtg agtacacaga agtcaagaac |
| 139441 | tgaagtttgg aaacctctgc ctagatttca gaagatgtat ggaaatgcct ggatacccag |
| 139501 | gcagaagttt tctgtagggg tgaggtcctc atggagaacc tctgctaggc agtgtgaaag |
| 139561 | ggaaatgtcg ggtgggagcc cccacacaga gtcccaMctg gggcaccacc tagcggagct |
| 139621 | gtgagaagag ggccaccatc ttccagatcc cagaatggta gatccactga cggcttgcac |
| 139681 | cttgcacctg gaaaagccgc agacactcaa tgccagccca tgaaagcagc caggagaaag |
| 139741 | gctatactct gcaaagccac aggggtggag ttgcccaaga tcatgggaac tcacctcttg |
| 139801 | catcagcatg acctggatat gagacatgga gtcaaaggag atcattttgg agttttaaga |
| 139861 | tttgactgcc atgctggatt ttgaacttgc atggggcctg tagccccttt gttttggcca |
| 139921 | atctctccca cttggaatgg ctgtatttac ccagtgcctg tacccccatt gtatctagga |
| 139981 | agtaactaac ttgcttctga ttttacaggt tcataagcaa aaggacttgc cttgtcttgg |
| 140041 | atgagacttt ggactgtgga cttctgagtt aatgctgaaa cgagttgaga ctttggaaga |
| 140101 | cggttaggaa ggcatgattg gttttgaaat gtgaagatat gagatttggg agggggcaag |
| 140161 | ggcagaatga tatggcttgg ctctgtgtcc ccaggcaaat ctcatcttgt agctccccta |
| 140221 | attcccacgt tgtgggaagg acccggtggg agatgactga atcatggagg tgggtctttc |
| 140281 | ccatgctgtt ctcattctag tgaatgggtc tcacaagatc tgatggtttt aaaaacaaga |
| 140341 | gtttctctac acaagctatt tgcctgctgc catctgggga agttgaggct gcagtgagct |
| 140401 | gtgatcgtgc cactgcagcc agggtgacag agtgagaccc tgtttaaaaa aaaaaaagaa |
| 140461 | aagaaaagaa aaaaaNgtaa gaaagagaga gagaggtaaa gagagacatg tagccctcac |
| 140521 | tacgctcact atgcttgagc acctggatgt agctatgcct gaagccagat aaaccctggg |
| 140581 | atttactggt acatgagcca actttccttt ttctgcttaa gatagtttga cttgagcttc |
| 140641 | caatgtttat gactaaagga attccaacta tctcttggtt attttaataa tttatatatt |
| 140701 | agttttatga tcataaaata aagtttttaa gagaagaagc ctggctaaag tttctttaag |
| 140761 | ttagtatacc tatcaatagt atgaggaagc agaaaaagta ttgaacaRgg aaatcaggca |
| 140821 | acctagattc tctgctggac ccacaaaaat tgctgtatga ctttgaacaa gtttcttaaa |
| 140881 | ctgtctagac tccaataaaa taaagaatca gagttagtga tatctaaggt atcgtatagt |
| 140941 | gctaaaagtc tttgcttcta taaaagaaga cctaattagt ctgatataaa ttgttcttaa |
| 141001 | ccaagacttt ttatagaaat gttaagaata aagactaata atttaaaaca tgtcaagcac |
| 141061 | ttataataaa accctttaaa ttgacaactt acatatgaga cgtcctcata tatacatgca |
| 141121 | aaatattcat tgcatcactc actggttaaa ttggaaaatt tagaaacaaa aattgagaac |
| 141181 | agcccggtta gacaaattca tacaatggaa tattatgtaa ttacaaaaac gaaatgaaga |
| 141241 | aactttatat tttgctgtgg acaatatcta aggcacattt ttaattgaaa agtgcaaaat |
| 141301 | ccaaagtggt tgtgctgtgt aaaaagagga gggaaagtca gatagattta attcctggtg |
| 141361 | caWgcatgga ttatctcttg aaagaatctc aaaaaaaaaa aaaaaaaaaa aaaaaacctg |
| 141421 | ctaccactga ttacctttgg agagactggg tcttattttt catgatacag tatatccttt |
| 141481 | tatcttttga atgtcaaaat atgtgactgc caggcacagt agctcctgcc tgtaatctta |
| 141541 | gcactctggg agaccgaggc aggaggatta tctgaagcca ggagtttaag actagcctgg |
| 141601 | gtagcatagt gagaccctgt ctctacagaa aagacaaaat tagccagaca tggtRgcatg |
| 141661 | cacttgtagt ccccacacac tgaggaggcc aaggtggaag gagtgcttga gctcaggagt |
| 141721 | tggaggctat agtgagctca ttgcaactac tgcacaccag actgggcaac agagcgtgac |
| 141781 | cctaccctct aaacaaaaat taaaaaataa aattaaaaaa actgtacatg taactgtatt |
| 141841 | acctactcaa agtcaaataa attttggaag tttttaattt aaaaattaca taaggctttt |
| 141901 | ttggtactta tggtcatgtc agggtgacat gtagcctagc tacctgtaag tcttcgagac |
| 141961 | cagcatttct cacacttcat cgtgcattct gaggatgcac tccaggggat cgtgataaaa |
| 142021 | tgcaggttct gatgcagcag gtctggagca aggaccaagc ttctgcacgt ctgacaagct |
| 142081 | ccaggtgatg ctgccgctgc aggccaggac cgcactttga atgtaggtct agaaccacac |
| 142141 | tgatcaatac ggtcaccact agccacctgt ggctactgag tgcttgaaat gtggctagtc |
| 142201 | caaaaagaga tgtggggatg tgtaaaatac acaatggaat tgaaaaagag tatcaaaaaa |
| 142261 | gatcataaat gattttatat cgatgacatg ctgaaatgat gtttttgata tgttgggtta |
| 142321 | aatggaatat attattgaaa ttcatttccc ctgtttcttt ttcctttttt taatgtaacc |
| 142381 | actagacaaa gtaaaacaac acacatggct tgcattatat ttctattgca tcatgctgtc |
| 142441 | taggagaacc tgatctccta caacctgcaa caaggcaggg gttgggacaa ccttgtcatc |
| 142501 | accctggagt atttgcaagg gtctgtaaat tggtataagg acactggaga cttgggagag |
| 142561 | agcacattga agaatctggc acccactcac ccctaacaaa taaaacttct atttaagtga |
| 142621 | aatacggatt gaagcgaaca atcaaagcag tcgtaaccct ggaactgtag gtagagcttt |
| 142681 | tgcagagcag agtcagccta agggaccagt gttgMaagga aggagtgagg tacctttgcc |
| 142741 | aggcagccag gcttgacagt ctgcaatgct gtggtgacag tgggaggaag tatgtcaact |
| 142801 | ctacttgcac cactgaacag cactgtccct accaccctga gtcacatccc agcctggcac |
| 142861 | cctcaaaaac aaagatgcgt ttaaagctaa gggcctgggg taatcacttg gcttgctgca |
| 142921 | acacagacac aggtctgaag acagccaaat agtcagaagt gtaatattag atcagcatag |
| 142981 | gccaggcatg gtggctcatg cctataatcc cagcactttg ggaagccaag gcaggcagat |
| 143041 | cgcttgaggc caagagttca agaccggcct ggccaacatg gtgaaacact gtctctacta |
| 143101 | aaaacacaaa aattagctgg gagtggtggt gcacatttgt aatcccagct actcgggaag |
| 143161 | ctgaggcagg agaatcactt ggactcggga ggcagagtct gcagtgagcc gagattgcac |
| 143221 | aactgcactc cagcctgggc aacaaagcta gaYtctgtct caaaaacata tatatattag |
| 143281 | attagtatag acataacgcc accaattcct tctgtgtggc cacagagcaa tctgcatcac |
| 143341 | acgggtaaat tgtaatctca aactcttcca tcctcatcca aggtaagtgc aatttttagt |
| 143401 | gaattggccc ctttttgtgt aagatgctcc aaagctcata cagggtaagg ggtagagggt |
| 143461 | ccacggtgta ttatcttctc cttagaaaca tacaaactgg gccaagattt ctttacttga |
| 143521 | ggccacaaga aaagtatcac atgacccgaa agagctgcac atccttcagt tctgttggga |
| 143581 | aattcatcct gcacctgggc tggggtgcag cctttctttc ctccctgggg tggcaaaggg |
| 143641 | aaggcatgag acacccagag gggagaaaag ctcaggtgga cacgtggtca tgtttctact |
| 143701 | cagcaggcac agaggtgcac ccttaagcat gatgtgggca tgttccctct cccagaaaac |
| 143761 | ttagctctat ggacaaagga gcaaaccaac cttgtcctgg gcccagccca gtcctcccac |
| 143821 | ctttcccatc acagacccag ccaagctcca gagggcactg accaggtttt atctccagca |
| 143881 | gccgcagttt tggatcttca ggtgggtgca Rtcgtctctt cttgcgccag cagcgggcag |
| 143941 | ggtatgtata cagctggccc ggggcaaggc ctgtggacag agacaatata aagattaaag |
| 144001 | gctccaggct ggaaggggcc ttggaaacta ccctgactaa cccYgtcatt cacaaggaga |
| 144061 | ggaagctgaa gcccagagag aagtgacttg tcaacatcac aaagctgatt gctagtggtg |
| 144121 | gcaaggctga atctaggtca tctgacccca ggaccctggc tctgtcagct ccaccatgct |
| 144181 | gttcagcaag cccatgccaa cgagagccat tgccttcacc aaaaccaatt ctgctctcct |
| 144241 | gcctggagag atcaccaact ggaccttcag acaagaagga cctttgcagt gtctccaaag |
| 144301 | cagtggatct ctatccactg cccctctatc ctgatccagg ttaagcaggt tcccttcaaa |
| 144361 | acggagcagc agtcagttgg cctactgtat ggacagcctg ggttgagaag catcgcaaag |
| 144421 | agacagaacc aaaaacacta gatctactca aaggcgtctc ccacccactc ccagctgctg |
| 144481 | gcagctcact gtaggcaaat ggaggcttgt gtagccccca tgagaacata aacccactct |
| 144541 | ggtgaccagg ccacatgacc tgcagagccc gcacacctct atatccccgc ctacatgcct |
| 144601 | gggccttaca aaaaaaaaaa aataggcagg aggccagggg ctgggcttac gtgccaccct |
| 144661 | gtgtgtgtgK cctttgacac caacagcatc actgtcatgc caagcaccat gaggagcctt |
| 144721 | cctgctcacc cgacaacaat tgtgtgtggc tctggtggca gccacgggcc ttagaggaac |
| 144781 | agggacttct ccatgccagg tcacagacta ttcccaggga aatggggcgg ggaagaggct |
| 144841 | gaggggtgag gaggcaggta tttgagactg gctaggtaat tttcagactg gctaggtgat |
| 144901 | ttgtagtcct gaagagagtg tctgataccc cccataataa accagcaagc caacaaatcc |
| 144961 | Ytaataggaa gaaaagccta atgaagacag ggactttttt gtcttgttct tgtcagaagc |
| 145021 | cccagaatca gaactgtgcc tgacacagag cagaaccaaa ctaaatactc attgaattaa |
| 145081 | tgaatgaata tattgcagct tcctttaaaa tcacaactct gtcttctgca ggtaaatgtg |
| 145141 | gcacatggca gctgttggac gcaggcggcc taccacaaaa gtgtcactgt gcacacctag |
| 145201 | gaccatggat tcagaaaaca tcagcacctg attcccatct gccgtcttcc attttagggc |
| 145261 | ccttaaaagt taagaaggga aactggcttc atttttcacc tttgcagaga aaatctatgt |
| 145321 | aactttcatt gctatatact cattcattca ttttcatttt taccagtcaa ctctacccat |
| 145381 | ttattgggat ggggccaaaa caaagtcaat tatcccagca ttaaaggaac caaaatactc |
| 145441 | aggtctcttt atgtttaatc acacagctgc tccacaggtc acaagattgt gaacaagttg |
| 145501 | gaggttctga ccaccccacc cctttggttc ttagaaacta gaactgacct ggactcagaa |
| 145561 | atgtcagaat tgaatgacca ttggccctgc tgttcctggc tccaggggac tcatccaccc |
| 145621 | agatccaaag catccaagag ggactccaga agtggaggcc caggcctgag gccagagccc |
| 145681 | cctgcacccc ctctcccacc atggccatct ctgggcctta ggtttccttc gttccccagt |
| 145741 | ggctgcttta cggtgcttcc aacctaatca aacacaggta agtaagagtg agctggcccc |
| 145801 | caccagcacc ctccaaaggt ctacgtggca agagtgcaaa agtgtccaca cttgtccagg |
| 145861 | tcctacaccc tccctgagac agcagtcact gtgcttggtg gtcagcggta ccagagaagc |
| 145921 | cccaggccac tctgcttgga gctttagacc cattcttctg ggagaattca ttttagcaac |
| 145981 | tagtttgaat ctccagaaga aggggctgtt tttgttccat tctttctctg ctggattgga |
| 146041 | gacactctgc agtgcaaagg aagagcacgt gagtcctggg aagcatccta acacctttct |
| 146101 | gagcctattg cctcacctgt aagataaggg cattgataac attaatacct acctcctggg |
| 146161 | gctgttatgg ggatttgctt atattcaaga gaaagctctc tatatccaca aagatgacaa |
| 146221 | atgtcagttg ttaattgtca tgagttagct gacctagtct tggactccac actcatgcaa |
| 146281 | catgacagac cccctgcccc atccaccatg ctgccacttg gttctttagc catgaaggtg |
| 146341 | gaaacaggct tctttttgat gtctgctttg aatccacctc atcacaacaa gcaaatggtc |
| 146401 | gcaagagctc tcttggagct ccctggcccc ttccttgccc tcctcaacag gctgacaggc |
| 146461 | accagggttc ctttatagga aaaacacgtt cagccctgga gttccactta cagagggaag |
| 146521 | agcagggccc catgagactt atgttgttta tctcagaaca ggaggcagat aactcaaagc |
| 146581 | atgtgtgtgt gtgcatatgt gtgcgtgcat gtScatgtgt ggtagatgga ggtggggaag |
| 146641 | gaggatggag tcacagctgc tgtcagggaa attaaccttg gggagtgagc ctagggctgg |
| 146701 | ggaggggaca tgaaggaggg gaaacaagtg gccaaagaca agaggggaaa ccagtcctcc |
| 146761 | ctggtaacca aggccactgc cacccctcac ttcacatacc ccactgagca aactactttt |
| 146821 | ctgttacttt gagctgcaac tcgagctaca acattcctgc ccatgtgtaa gcacaaatgt |
| 146881 | gatgcacaaa gacacacaca cacaatcaaa aactcacaca cttgctggga gaactggctc |
| 146941 | catggaaata cctggcattg aaaggcttga tgcctttatg gttagcagac tccagacaaa |
| 147001 | tacagctggt tatgcacatt gcacaagttc agcaggtgga tcctgagggt tctgggcatc |
| 147061 | tctcacagac aaagcatgag ctattcagaa tttagaagat gaaatgggag agaaaagaga |
| 147121 | gggaagagtg gccgggtaca gtggctgacg tctgtaatcc cagtactttg ggaagccaag |
| 147181 | gccagcagat cactgaagcc caggagttca agaccagcct ggccaacatg gtgaaacctc |
| 147241 | atctctatca aaaatacaaa aattagccag tatggtggcg catgcctgta atctcagcta |
| 147301 | ttcgggaggc tgaggcagga gaattgcttg aacccaggag gtggaggtta taggagctga |
| 147361 | ggtcatgcca ctgcactcca gcctatatga cagagcaaga ttctgtgaaa gaMagaaaga |
| 147421 | cagaaagaaa gaaagaaaga aagaaagaaa gaaagaaaga aagRaagRaa ggaaggaaRg |
| 147481 | aaggaaaNaa agaaagaaaN gaaaaaaaga aagaaagaaa gaaagaNaga agaNaagaga |
| 147541 | agagaaaggg agagggaaag agggggagag agggaaagag ggggagaagg ggagagaggg |
| 147601 | agagaggaga gaagagggga agaaggggag agagggagag agggacagag ggggagaaag |
| 147661 | ggggagagga aggaaggaag gaaggaaggg aggaagggag agagggaggg agggagggag |
| 147721 | gggagaagaa tgatatggga taggaacagc aattgaaatg gggaagaaaa aaactgaagt |
| 147781 | agtttcatct ttccccatcc agagctgaac tgtggcagtt caagcccccc agggaaaggc |
| 147841 | tctctctctg caggatctca gagaggtgga cccacagctg cccctgaaac ttcctttcaa |
| 147901 | tggagaattt attatgtcac aggacacagt gtcccactgt cggccaactc ttactgtgat |
| 147961 | aaagagattc accatgacct tattttgaat caaaatctat ctccctataa gttataccaa |
| 148021 | tttgtccacc ctctgttcca acagcaaaca gctacatccc aatcctgcat cccatatatc |
| 148081 | tgcttgggtt tgaagggcag ctaaccaaga agccgaggaa ctaaagagga ataagagttg |
| 148141 | taggaaagag ttagtgacta cccctgctca gaaccgggta acaggatcaa agtttccaaa |
| 148201 | tagaaaatga caatacaaca atttaaaata caatttaaaa aaatacaata taatacctat |
| 148261 | ttacatagca tttacattgt attaggtatt ataagcaatc tagaaatgat ttaaggaata |
| 148321 | tgggagaatg tgtgtaggtt atatgcaaat actatgccat tttatataag ggacttgagt |
| 148381 | acccacagat tttggtatct tctggggtcc tagaaccaat tcccagtgga tactaaagga |
| 148441 | tggctgttat gtatataaca tactatcatt tatttaacaa atggggatac acacatattc |
| 148501 | agacaaatat atgtgtgtgg tgtgtttgca tgtttttttt tatgggggta taagccaaaa |
| 148561 | gctaaaaaaa attacctaga gagagagcaa aaataaaata atagtagcaa gaaaaagaga |
| 148621 | aggacgaaga ggaaattgcc tatacgaaaa agaaacaggg taaaagtgaa gatacagtta |
| 148681 | gactttctca taatatacca ttggaacaat gcaaatattt tacacaatta taaagtaaaa |
| 148741 | ttaaattaaa ataaaaagca tacatccgtt tctgactaaa atggagtata aagaccagat |
| 148801 | ttagctccca cctaaaacca aaagagagga agaaaaaaat caacaacagt tttgaagaca |
| 148861 | atgaacatca ggcattgaag gacagtgatc cctgagacac tgaaaacaag caagataagc |
| 148921 | cctgtgattg ccccagctta cagccttaag agagtctcca gactggaaca caaggaggag |
| 148981 | gaatacaggt ggagacctgc agattcccca cgtttgagtt gaagagaaaa tgccaagagt |
| 149041 | ctgaggagac cacagtgact agaacccaag gaactgaagg gtaaacaaaa gagagctgca |
| 149101 | cagagaatgt ggagatccag agggtccctt tcaagtattc agcttagcgt tgatccattt |
| 149161 | atgcctgtga ggaaactaga taggactggg aaaagaacta cccaaaaaga ttagaggtaa |
| 149221 | cagcgctcgg tgtgcacaag ggggcggaaa tagtctgttt ccaccagcca atatggaaaa |
| 149281 | ctgtatgatt cccggggtac tgggtagagt acagtagaaa gatcttgcct cactaatagg |
| 149341 | gaatacttag ccctagacta agtactgctc tggctccacc taacaaatct taaaagcaag |
| 149401 | acaagaaaac atcaaactgt tttcaaataa cttaaatatg tcccaaaaca atgttcaaga |
| 149461 | acatttatag gaataaaata tatctacagc attcagcaag gtaaaattca caatgtgagg |
| 149521 | cacccaaccc aaaattacaa ggcatgtaaa gaagctggaa aatatgactt atMataagga |
| 149581 | gaaaaatcaa tcaattgaaa ccaacccaga actgaaactg atattagaat tagtagcaag |
| 149641 | aacattaaaa cattatttgt atgctctgtg ttcaagttta gtaaaaattt gggagataca |
| 149701 | tatgtatttt taagactcaa atttaacttc tagaaatgaa aactataatg tgcaagatga |
| 149761 | aaatcatatc agagggagtt aatgtcagat tagatattgc agaagaaaag acagataaac |
| 149821 | ttgaaaacac agcaatagaa actattccaa aatgaaaccc acaggaaaaa acagaaaaga |
| 149881 | aaaaaaaata atggcataaa tgagctgtgg ggcaacttca aacagcctaa tatatgtgta |
| 149941 | attgaagaaa gggaagaaaa aatatttgta gaaataatgg ctggaggctg ggtgcagcgg |
| 150001 | ctcatgcctt taatcccaac actttgggag gccaaggtgg aagaatgcca taagcctggg |
| 150061 | agtttgagag cagcctgggc aacatagtaa gatcctgtct ccacaaaaga gagagagaga |
| 150121 | gagagagatg aaagagagac agagagagac agagaaaggg aggaagggag agagggaaga |
| 150181 | aagggaagga aggaaggaag aaagaaagaa tttaaaaaga aataagggct ggaaatttct |
| 150241 | gaaatttgat gaaaactata aacccatagg tctaagcagc tcaataaagc taggtacaat |
| 150301 | aaacattaag aaaactacag caaagtatgt cataatcaaa actgctcaaa aatagtgata |
| 150361 | aagagaaaat attaaaagca accagagaaa ggaaacatgt taacatatac aggaacattc |
| 150421 | ttatctttgc aatgcaagca agaaaagagt ggttcaacat ccctacaata ctgaaaggga |
| 150481 | aaaaaaaact tgtcaaacta gaattctata cccagctaaa atatctttca agaactaagg |
| 150541 | caaactaaag atagtttcag atatacaaaa gctgaaataa ttcatcatca acaaacccac |
| 150601 | actctaagaa atgttaaaaa gaagtacttt aggcagaagg aaagtaatac cagacctaaa |
| 150661 | tttggatcta cacaaaggaa tgaacagcat cagaaatggt ctagtacatg aataattata |
| 150721 | cgagatgttt ttcttattat tttaaactca ttaaaagatg atcaactatt tttaaaaata |
| 150781 | caatgatggc cgggcgcRgt ggctcatacc tgtaatccca acaatttggg aggccaaggc |
| 150841 | aggcacatca cctgaggtcg ggagttcgag aaccgcctga ccgacatgga gaaaccccat |
| 150901 | ctcgactaaa aatacaaaaa ttagccaggc atggtggcgc atgcctgtaa tccatcaaca |
| 150961 | catctactct gactctgaac tcctcctgcc ttccttttat aagaaccctt gtgattacat |
| 151021 | taggcccacc tggataatcc aagattatcg caccatctca aaatccttaa tcctgtcttc |
| 151081 | gaagtccctt ttgccatgta aggtaacata cgcacagatt tcagtaatta aaatgtggat |
| 151141 | gtctgaggtg aggaggggcc ttattctatt aatcacagta ctattgtaga ctccttatac |
| 151201 | tattcatgaa atagaataat ataatttgaa agttaactat aattaaagat gtacactata |
| 151261 | aacactaagc aatcactaaa ataacaacaa aacaagagtt atagttaata aaccaacaaa |
| 151321 | aaagagagaa aacataatca cattaaatgt aaattgccta aataccccta cttaaaaaag |
| 151381 | attgtcagat tacatgtaaa aagcataaca caactatatg ctgcctatta aaaaactact |
| 151441 | tttaatataa agatgcaaat aggcaagaag taaaagaatg gaaaaagata tttcatgtta |
| 151501 | atattaatca aaagagagct ggagtggcta tattggaatt agacaaagga gatttcagat |
| 151561 | aaaataatat taatagagat taagaggatt attttataat gataaaggaa tcaatttatt |
| 151621 | gaggatataa caatactaaa tgtttataca cctaataaca aagcttcaat atacatgaaa |
| 151681 | caaaaagtaa tagaattgta aggagaaata agcaaaccca caattatagt cataagtttc |
| 151741 | aataccctac ctctcaatga ttcatagaat aaatagaaaa taaggatgta gaagatttaa |
| 151801 | aaaacattat caaccaattt gacctaattg atatttatag aacattccaa acaacaacag |
| 151861 | caaaacacaa ttcttttcct gagcaaacag aatatttacc aaggtagacc acatttggtg |
| 151921 | ccataaaaca agttgcaata aatttaaaag aatttgaacc ataaaaatta ttctctgacc |
| 151981 | atttggaatt aacatggaaa taaataatgg gcacaacttt ggaaacgtcc caagtgtttg |
| 152041 | gaaactaaac aatacagttc taaataatcc atgagtaaaa gaagaaacca aacagaaaaa |
| 152101 | tattttgaac tgaatgaaac taaaaaccct atatcaaatt ttttaaatga ggcttacata |
| 152161 | gtatttatgg ggaaattata tcagaaaatg atgtagtaga aaagaaatat cttaaatcaa |
| 152221 | tgacctgttt acaccttaag aaatagataa acctactatg taccataaaa attaaaaaat |
| 152281 | taaaagaaat aagaaacaga agagcaaatt aaatccaaag taaatagaag gaaatataaa |
| 152341 | gatcagagca gaaaaaacaa ttctaccaaa agctatttat ttgaaaagat caataaaatt |
| 152401 | gataaacctc tagtcagaat gatgaggtaa aaaataacat tcaaattaac aatataatga |
| 152461 | gtaaaataat atgccaccac agattctaca tatattaaaa gagtaatgca gcaatattat |
| 152521 | gaacaacttt gtgcaaccaa ttaggagaat gtagataaaa gaaacaaaat cacatactaa |
| 152581 | caaagcttac tcaagatgaa atagataacc taaatagccc tatatctata aaataaatta |
| 152641 | aatttataat taaaaacatt acctcaagga aactgccaga tccaaatgga ttcactggta |
| 152701 | aatgttataa aatatttatg gaagaaataa taccaattct atataaagtt tcagaaaagt |
| 152761 | aaaaatgaag aaatacttcc caactcatta tatgagacca gcatcaccct gacacccaaa |
| 152821 | ccagacagac attacaagaa aactatagac taatatctct cataaacata gatgcaaaaa |
| 152881 | ctctaataaa acttgagcaa catgaatcta acaatacatt aaaaagataa aatagcataa |
| 152941 | caaagtgggg tttatcctag gaatgtaatg ttggtttaac attcagaaat caatcaatgt |
| 153001 | aactcattat gttaaatgta aacttaagat attaaattta atcaaccatt cctacataat |
| 153061 | gaagcttcca tgaacaaaag aaaaaccaca tgatcatctc aatagacaca gaaaatcaga |
| 153121 | tgacaaaatc aaatatccat ttgtgattta aaaaaaaaaa ctctcaaact agaaatagaa |
| 153181 | gggagcatcc tcaatctgat aaagaatatc tacaaaaaat tctacagcta atgtcatact |
| 153241 | taataacaaa aaaataaata aatgctttct ctttaagatc aggaacaaca taagggtgtc |
| 153301 | cagtctcacc acttttacct aatattatgt tggagagtct agccagtgca agagacaaat |
| 153361 | aaaataaata aaagttattc atattaaaag aaagaagtaa gttttcttta tttactattc |
| 153421 | aggttatcta ctagcaaact aacagataat caactatata gaaaatgcta cagaatctat |
| 153481 | agaaagctat tagaactaat gagtttagca aggttgcagg atataggaag aatatactaa |
| 153541 | attaattgtt tttctatata ttagcaaaca tcagaaatta aaatgtataa aaatttattt |
| 153601 | ataatagtat caaaaagtat taagtgctta gagacaaatc tgacaaaaat gttaaagatc |
| 153661 | tatacactaa aaattaaaaa tcattgatga gaaaaattaa agaagatatt aataaattga |
| 153721 | aagatatgcc atattcatgg gttgcaagac tcaatactga aaattgtcaa ttttccccaa |
| 153781 | atggatctat agatgcaaga caatcccaac caaactccta gccagttttt tgtagaaatt |
| 153841 | gacagactga ttataaaatc catatagaag tgcaaaagac ctagaataat caaaataact |
| 153901 | aagaaaatca agaacaaatt tagagggcta acaccacctg atttcaaaca ttataaaact |
| 153961 | ataataatca agacagtgtg gtaagagtat aaagatagac aaataggtca ataaaacaga |
| 154021 | actgagagtc cagaaataaa cacacatata tgtggacatc taatttttga caagccaatt |
| 154081 | aagtggagaa agacgagtct tttcagcaaa ctatgttgaa acaattgaat atccatctgc |
| 154141 | aagaaaataa actaatgcat actttgtaYc atatacaaaa ttaactcaaa atgaatcata |
| 154201 | gacctaaatg tgaaacctaa aattataaaa ttttaggaaa aaaaaataag agaaaccctg |
| 154261 | accttgcatt aggtagtaat ttctcagaca taacatcaaa agcacaattc atgaaagaat |
| 154321 | aaattgataa actggatttc accaaaattg aaaacttctg ctctttgaat gagactgcta |
| 154381 | agttaatgaa aatacaaacc acagattgta aaaaaaaatt tgcaaagcat aggtctgata |
| 154441 | aacttattgt atctagaata tataaagaac aatcaaaatt caataataag caaatagctc |
| 154501 | aattttaaaa tggacaaaag atttgaactt taccaaagaa catatatgga tggtgtaata |
| 154561 | ttgtgatata atgagaaatg tatattttgg tcttaatccc cacttcctgg cacacagttc |
| 154621 | ctaaaacccc tggcatctcc aagtgatgtg tctttttata tgctaatgag atgaccctgg |
| 154681 | ctgggacctg ctggatagcc tctggataag ggattataaa cagcggagcc aaccacaaga |
| 154741 | ttagcaggct gtacctttta gccccaactc taacttccca cctctgacct ccaggaagaa |
| 154801 | aagaggcact agtagttgag ctagtcacta atggccaatg atttaatcaa ccattcctac |
| 154861 | ataatgaagc ttccatgaaa atccaaatgg actagattca gagatctttg ggttgatgaa |
| 154921 | catgtggggg tgctgggagg gtggctcacc tggagaaggc atggaagctt catgtctctt |
| 154981 | ttccccttac cttgccccgt gtacctcttc catttggctg tttcagatac atgtccttcg |
| 155041 | taataaactg gcattYtagt atgcaaacta ttttcctgag ttgttttttt tttttttttt |
| 155101 | tttttttttt tttgaggcgg aatgtcgccc aggctggagt gcagtggcac gaactcggct |
| 155161 | cactgcaagc tccgcctccc gggttcacgc cattctcctg cctcagcctc ctgagtagct |
| 155221 | gggactacag gcgcccacca cgcccagctt attttttttg ttgtattttt tagtagagat |
| 155281 | ggggtttcac cgtgttagcc aggatggtct cgatcttctg acctcatgat ccacccgcct |
| 155341 | cagcctccca aagtgctggg attacaggcg tgagccaccg cgcccggcct gttttcctga |
| 155401 | gttccagcaa atgatcaaac atgaggggca gtcatggaaa cctccattta tagcccatag |
| 155461 | gtcagaagca cagctgacaa cctggacttg aaaYtggcat atgaagttga ggcagtgctg |
| 155521 | tgggctgagc ccttagtctg tggaatctga cgatatctca cagtagatag tgtcaaagtt |
| 155581 | gagttaaact gtggaacacc cagttggtgt ccgcagggaa ttggagaatt gcttggtgta |
| 155641 | ggggaaaaga catccacaca tctgctcaca gaaatattga gtaaaagtat agagaataaa |
| 155701 | acagtttatt ttccctcaca gatggcaaat aagcacatgt gaagatgctc aaaatcatta |
| 155761 | ttaactaggg gaatgcaagt tgaaactgca atgaaatact actatatatc tattagaatg |
| 155821 | gctaaaaaaa ttttcaaact gaccacacca agtgttggca aggatacaaa ggaagtggaa |
| 155881 | ctctcatata ttgctggagt caatgtaaaa tggtacaacc actttggaaa agagcttggc |
| 155941 | agtttcttta atagtgaaac atctatctct cttatgatct agttgttcta ctcctaggta |
| 156001 | gttaacctct caaggagaaa ggaaagcata tgtccattca aaaacttgta cacaaatgtt |
| 156061 | catagtggct tgatttatag tagtcaaaga ctgcaaacaa atccaaatgt ccatcaacag |
| 156121 | gtgaatggat aaattaactg tgatacagcc atgcaatgga ctactactca gcataaaaag |
| 156181 | gaattattga tatagtatta attagtaatt atgctaagtg aaagaagcca gacatgccct |
| 156241 | tgtccctaaa aaagggagta cacaggccgg gtgcggtggc tcacgcctgt aatcccagaa |
| 156301 | ctttgggagg ccgaggcggg tggatcacga ggtcaggaga tcgagaccat cctggctaac |
| 156361 | acggtgaaac cccatctcta ctaaaaatac aaaaataatt agccggggag gctgaggcag |
| 156421 | gagaatggcg tgaacccagg aggcggagct tgcagtgagc agagattgcg ccactgcact |
| 156481 | ccagcctggg caacaaggcg agactccatc tcaaaaaaaa acaaaaggga gtatacagtg |
| 156541 | tgtgtttctg gtatagaaag caaatcaatg gcttctctgg ggagggagta aagaagggta |
| 156601 | gaggcatgga gaagcgcagg aaggatagat ttctaaacac ataggaaacc tttgcgagtc |
| 156661 | atggatatgt tcattatctt gattgtggta atggttccac aggtgaatat atgtcaaaac |
| 156721 | ttatcaagct ggacacatta agtatgtgca atttattgta tgtaattata cttcaataaa |
| 156781 | gctgttaaac ttttttaata acaaggcaat ctcagggact tctgcttctg gaaagatgaa |
| 156841 | atagatacac ttttttatat tcctctcact aagcacaact aaatatcctg gacattattc |
| 156901 | atagaacaga cataagaaat aactgaaaga tgaagggaac cagtcagacc agctagggac |
| 156961 | tttgggacct gagaaacaac agggtgggtg gtgagctccc tgggtattat tttatctatc |
| 157021 | tacctatcta tctagctatc taactatcta gctagctagc tatttattga gacaaagtat |
| 157081 | cactctgtag cctgggctgg agtgcaatgg cgcaatcctg gctcactgca gcctcaaact |
| 157141 | cctgtgcttg agcaatcctc ccacctcagc ctcccaagta gctgggacta caggcacgca |
| 157201 | ccaacacacc tggataattt tttaaatttt tatagagaca aggtctcact atgttgctta |
| 157261 | ggctagtttc gaactcctgg gctcaagcaa tcctcctcct ttggcctccc aaagtgctgg |
| 157321 | gattacaggc atgagcctgt gtccagcccg atcttgggtc tcctttatgc ctcatatatc |
| 157381 | ctggactgga ttctggaaaa gctgataacc cagaaacacc aacaaatgaa gaccaaaaaa |
| 157441 | gacctcaaga aaatcctgct ctctctagcc agagaaccag gaaaggacca gcctagcaag |
| 157501 | acaagcaggc atcccaatag ctttgctggg ttggtgtcag agaaggccaa gggaagagct |
| 157561 | ggtgccttgt ggtatcagta gaggccctcc tcctcccaag taggataata ccagtagagg |
| 157621 | cctagtgggg agcctgaact cccacctcca ctcagcaaca acaaggtgtt cttcccctgt |
| 157681 | taaagaggat gtcaacagaa gccaagtaga gaagatggat ttccacatcc atctgacagt |
| 157741 | aataagggga gcctcRcctc ccctactagc atagcttgaa aggagacctg ctaatataga |
| 157801 | agatttaaat aatatctagc gtctcataaY ataacagcca aaatgtccaa gattcagtca |
| 157861 | aagccactca tcttatgaag aaccaggaaa atctcaaatg aggaaagaca cagacaccaa |
| 157921 | caagaaaata acaaaaatgt tagaattatc tgacaaggat cttaaagcag ccatcattaa |
| 157981 | aagtgtttca acgagcaatt atgaacacat aaaacacatt cagcaaagaa atagaaaata |
| 158041 | tgaagaacca aatagaaatt tgaaaactga aaaaaattgt aacaaaaaat aaaaaagctc |
| 158101 | tcaatggatt ggcttaacag cagaaggcag aggacagaat aaagaagcaa tgaacttgaa |
| 158161 | gagagaacta tagaaatcac ccaagctgag caacagagag aagaatttta aaaataaatg |
| 158221 | aacacagcct cagggactta tgggactata acaaaagatc taacatttgt gttgttgaag |
| 158281 | tccgagaagg agaggggaaa gagggttgaa ctgaagaagt attcaaagaa ataatggctg |
| 158341 | aaaaattccc aaatttggca aaaaaaaaaa aaaaaaaaMc ccataaaccc acaggttgaa |
| 158401 | gaagctgagt aaactcaaac agaataaacc caaaaaatct atggcaaaac acatcaaaat |
| 158461 | cacgtttctg aaactaaaaa caaagaaaaa acttgaaaac aatgagagag aaatgacacc |
| 158521 | ttacttataa gggaaaaaag catttaaatg acagcagatt tctcatcact aaccatcaga |
| 158581 | aagaagtggc aaaacctttt tcaactgcta aaataaaagg actatcaact cagaatttta |
| 158641 | tatccagcaa aaatatcctc caggaatgat ggcaaaatca agacattctc cactgaagga |
| 158701 | aaactaagaa gttgttacca tcagatttac tctaaaagaa tggctaaaga aagtactaca |
| 158761 | tcagaaagta aatgctaaaa gaaggaatct tggaacttca gtaagtaaga aaaacaacga |
| 158821 | aggagtaaaa atatgagtaa atacaataaa cgcttctctt acgttttcta aattatgttt |
| 158881 | gatggttgaa gtaaaaatta taacactggt atggttttca atgtatgtaa aggaaatgct |
| 158941 | taagacaact atattataaa gggaggaggg taaaaggatt taaataatga gtgttgggtg |
| 159001 | gtaatagaat agttttgtat cttgattgta gaagtggtta cacaaatcta gacatgtgat |
| 159061 | aaaatggcat aaagctacat atacacattt taccaatgtc agttttttgg ttttgatatt |
| 159121 | gtaagatgta ttaatcttgg aggaaattgg atgaagaaga taggggatct ctattatatt |
| 159181 | tgcaactgca aattgcaaat acagtcacaa ttgcaattat taattatttc aagataaaag |
| 159241 | ttgatttaat aaagaagaaa ttatatgaat cccctcccca ctcaaaaagc aaaccctgat |
| 159301 | aagtgaaagc aaaatgaaac aaataaatcc aactgtgtat tgagttagtg gcacaatcac |
| 159361 | acagaggaac tatctcagtg accttaaagg agagtaattt ggctgcacat gtttaatgga |
| 159421 | atatacccta aggacaaaaa gacttgcaaa aaaaagaaaa tatcttaaac ctttttcagt |
| 159481 | aatcttattg ttaatagtca tattgtttgt tctgatactg tgtgtgagtg tctgtgtgtg |
| 159541 | tgtgtgtgtg tgtgtgtgtg tgtgtgtgtg taaaataaag caaataagta attacatcaa |
| 159601 | tatcatttct aggatttttt ggtgtaggag aaaggagata cagaattaag atacttaagt |
| 159661 | aaaaactgtt taaatctaaa ttgaatggga tgtatcaaca taatttcata atgtattttc |
| 159721 | tcttaaaaag aaaaagaaaa aaacaaatga atactatttc ttagttctag tcactgataa |
| 159781 | ggcctagaaa caatgaccaa gccaggagca atgaacagca ctgcggtctc taagtaactt |
| 159841 | ttcctacaaa caggagccag gatttcttca agaaatagct gatttcagtc ctggagagga |
| 159901 | ggaagYgtac aggatgagct tgaaatattt catcctaata ttaggatggc Wagaaggcca |
| 159961 | tcagaaacta ctggggttgt ttcaaaacac cagcactggg ccgggcacgg tggcttacgc |
| 160021 | ctgtaatccc agaactttgg gaggccgagg caggcggatc atgaggtcaa gagatcaaga |
| 160081 | caatcctggc caacatggtg aaaccccatc tctgctaaaa atacaaaaat tagcYgggtg |
| 160141 | tggtggcatg tgcctgtagt cccagctact cgggaggctg aggcaggaga atcgcttgaa |
| 160201 | cccgggagRc agaggttgca gtgagccgag atcatgccac tgcactccag cctggctact |
| 160261 | gagtgagact ccatctcaaa aaaaaaaaaa aaaaaaaaca catcagcact ggccagaatc |
| 160321 | ctgaatggcc agagatgagg caagataagc Ytcataagaa taatagctac actggattaa |
| 160381 | aatacatcaa atatgtttaa atctgtgagt tcctggtggt attcaaaaaa ataattatta |
| 160441 | ttatattgat cgtctttgga gaatgctagg gaaccaactc attattttga aaactggtaa |
| 160501 | atagagggga aaaaagtcat ttatcctgcc tttcatatgt gaactgtatc tcacaataac |
| 160561 | taaaatttta atgagggaat gtttttcttt atagaagttg tagagctaac aaatgaagat |
| 160621 | ataatgatag tcctggattg aatattaact ttttgcaacc cttaatggga taaagaatct |
| 160681 | aggcaattat catgaagggc tgctaacatc agtaaaagag gtaaccagac tgtatgagcc |
| 160741 | tcttgatgaa agtacacact accactcagg agttgttctt accaaaaaaa aatggaacct |
| 160801 | gaataaatct ctagacctaa ctaccaattc acagKaataa caccatggga acgcaaccaa |
| 160861 | atccttctgg gaaaccctac agcgtaagca acccagtgtc ttcaacaagt aacatgcaag |
| 160921 | gaggaaagag agagaggcag agacagagag aaacccagag atgttaaaag acacattaac |
| 160981 | cagttgcaat gtagggttcc catttggatc ctgatttaaa ctacaggggg aaaaatgaat |
| 161041 | gttggccaaa tatttgatga tattaaagtc ttattgctaa ttatttgagc tctgatagtg |
| 161101 | attgtgagtK tgtttgtttg tttaagttct ttaccattta gagatacata acaaaatatt |
| 161161 | tacagatgaa atcacgtgct tttgtggatt tgcttcaaaa taaaatgagg tcagggagaa |
| 161221 | aagtaaggag tgcgtataga tgaaacacga ttgaccatga gttgaacatt gttgaagcag |
| 161281 | ggctacatgg ggatgtgttt tactattgtc ttcagtttta tatgaaggtg aaatttttcc |
| 161341 | aaatgWaaca tttttaaaaa catatccagg cagagccaga aaaatcacat tcctgtagct |
| 161401 | gtctccctaa taaaaaggtc tttattctgt tctttgcagc ctagaagact agtgtcactg |
| 161461 | tagctgtagt aataacagta atatcaataa taataggcca ggtgcagtgg ctcacgcctg |
| 161521 | taatcccagc actatgggag gcccaggcag gcagatcatg agatcaggag ttcgagacag |
| 161581 | cctgaccaac atggtgaaac cccgtctcta ctaaaaatac aaaaactagc tgggtgtggt |
| 161641 | ggcgcacgcc tgtaatccca gcgactcagR aggctgaagc atgagaatca cttgaacccg |
| 161701 | ggaggcggtg agtcgagatc gcaccactgc actccagcct gggcaacaca gcaagagtcc |
| 161761 | atctcaaaaa aaaaaaaata acaataataa taataatacc cagcattttg gaagcacata |
| 161821 | cttgctatgt gctaaactca tttaagagtt tttataaatt ttctcattta attcagacga |
| 161881 | aatcttatgt ggtaagtact attattattc acattttaca gatgaggaac ctgagggaca |
| 161941 | gaagtttggt gacaaaggta agcctcacac ccagatctaa tggcagattt ccggcttgta |
| 162001 | accaacaccc aaaggtaaaa gatgaagatt gctgtcatgt cctcagtctt gccttctttg |
| 162061 | ggataaacac ctttagctct ttcactcccc accccacccc acccctgcca catggcttca |
| 162121 | atgccccaca ccatccttgt cacattcctt gaaacacctg ccaggagcca taaaagtagc |
| 162181 | tgagcttctt taaggtatct cagaggccca tgtgcagctt gccccacccc gatcctaccc |
| 162241 | aggctgcctg gtttcccaga caagctgcgg tcctcctccc tccaggctgg gagcctgcac |
| 162301 | atgcgcatgt cccaggagtg gcgcagctca cctgggcctc ggtgcctctt ctccatccag |
| 162361 | atgtagcagt tgttctgggc caccccagtc tgtgagtcca ggaagggaag acgcacgctg |
| 162421 | cgctctgcac acagccgtga gttgtaactc cggcagtgct caatggcttc cttgtagaac |
| 162481 | tggtccccga gcctgccaga gtcagagagt gaaggggtga ggccagggaa gactgaaaca |
| 162541 | cacccagagg gaaacacacc cagagggaaa cacacctccc tggaaagact gaagacctcc |
| 162601 | cagttatgaa gaccaaggca atgagcacca agagccttgc caatgagcag aatgcaactc |
| 162661 | aatgtcaggt cttgactaga ttttattttc tatcccaaat ccaccaggca acttagaaac |
| 162721 | tcctttaaca aagagcatgg gcttccaagg atgaatcacc agtaagagag agctgtccat |
| 162781 | gttctaggaa gcatgaggtc ccactccaga agggaagtag ctctggaaca accttctgca |
| 162841 | tggaatctag aggacatcac acttctagaa cctcctcact catattagct acattttatt |
| 162901 | ctcagaggtc accagcaaag gcactctagg tgaacccaaa tcaccagcaa agatactcag |
| 162961 | ttttccatta caacagaaga gaaacactcc atataaacaa aaactaggac agaaaaaaca |
| 163021 | ttctataaaa gtagggaaat tgcaggaaga agggggtgta gaggaacagg caccaatctt |
| 163081 | ttaaaaccat attacttaaa gaaaaataac tttccgatag aaaacaaaca gaattcaaca |
| 163141 | attttaaatt caaaataatt caactcatgg agtttaaaaa acaaatagaa agacctttct |
| 163201 | ggtttgctag actgagaatc tgattaagca cagtatattt tacgttcctg actctttggt |
| 163261 | atttttggac acgtggtgat gctactaatt ttaaggtagt gtccttttca aagaccaaaa |
| 163321 | atggagcatt gatataattg atcaaatgca aagcactaaa ataatcaatg agatcaaata |
| 163381 | cctaccaaat aaaacatagg cacaggttaa taaatgcctc aaagaaatac aaatgtaata |
| 163441 | acataaStta tgccaggtaa attcaagcct cttactggRg tcggggggca gtggacgcaa |
| 163501 | aggagtggag tgtgggaaga agaagtagga agggaaggag ccacagagtt cacaggggca |
| 163561 | aaagggagaa tcaagtgttt attctgaagc aacattatga tatctttttg ttttggttct |
| 163621 | atgtagattt cagaaagcca aaatgagagg ggaagtctct aaaaatatca tttcttaacc |
| 163681 | aaagtattta gaggaataaa aaaagcaaaa ttggggtgtt gggttttttg ggtttttttt |
| 163741 | tttttttttg agatggagtc tcactccgtc gcccaggctg gagtgcaacg gtgcgatctc |
| 163801 | agctcactgc aacctccacc tcctgggttc aaacgattct cctgcctcag cctcctgagt |
| 163861 | agctgggatt ataggtacct gccaccatgc ccggctaatt ttttatattt ttagtagaga |
| 163921 | cagagtttca ctatattggt caggctggtc tcaaactcct gacctcaggc gatccacccg |
| 163981 | cctcagcctc ccaaagtgct gggattacag gcgtgagcca ccatgcctgg cctcaatttc |
| 164041 | ttaattgtgg taaaatacac ataacataaa atttcccatc ttaagtgtac agtttaagtg |
| 164101 | tacagttcag tggcattaag cacatgcata ctgttgtacg accaccacta ctatccattc |
| 164161 | ccagaactct tttcatcttg caaaactgaa acactatacc cattaaacaa taactccccg |
| 164221 | ttccgcctct gcccccatct ccagcaacta ccaatctgct ttctatctct atgattctga |
| 164281 | ctactccaag tacttcataa aagtggagtc atacagtatt tgtctttttg tgactggctt |
| 164341 | atttgactta gcataatatc ctcagggtta acctatRtta tagcatatgt cagaatttcc |
| 164401 | ttccttttta agtcagaata atattttaag tcagaataat attttttaag tcagaataat |
| 164461 | attccattgt atgcatatac cacattttgc ttatccattc atctgtcagt ggatatctgg |
| 164521 | gttgcttcca cattttagct attctgaata atgctgctat gaacatgagt atacaaatat |
| 164581 | cttttcaaaa ccctgctttc aggctgggcg cagtggctca tgcctgtaat cccagcactt |
| 164641 | tgggaggctg aggtgggtgg atcgcctgag gtcaggagtt cgagaccagc ctggccaaca |
| 164701 | tggtgacacc ccgtctctac taaaaataca aaaattagct gggcctggtg gcagacgcct |
| 164761 | gtaatcccag ctactcaaga gtcgcttgaa ctcagaaggt tgtggtgagt tgggattgtg |
| 164821 | ccactgcact ccagcctggg cgacagggtg agactctgtc taaaaaaaaa aaaaaaaaaa |
| 164881 | aaaaaaaacc tgctttcagt tcttttaggt atatactcag gagtagaatt gctgagtcat |
| 164941 | atggtagctc tagttttcat tttttgagga accaccatac tgtttttcat ggcagctgta |
| 165001 | ccattttcca tttctaccaa cagtgtacaa gggttccaac tactccacat tctcatcacc |
| 165061 | acttgttaga aagcaaagtt ttaaatgcac ttttctatac tctttttaaa aagtcaacgg |
| 165121 | gtcaggttct aacaaagagc ttaaagcaaa tatcttactg tgggaaaaca caaccctgga |
| 165181 | agcttctgag gcatcatttg gaaattactt gttgacaagc atccaaaaaa aaacaaacaa |
| 165241 | caaaacaaaa accctttgct cctttttcac ctctgaacat cttcacattc ataagggagg |
| 165301 | cataatacgc tcagaaagtg tcctggcagt tgcaaaacca aactgtaatc aaattgtaat |
| 165361 | agtcacgcag ctggaaatga attggggagg ggagatgaaa gcccattaca atgctataaa |
| 165421 | actgaatggc acataagctg atgtaatcaa agctggtaac aaggatcatg agattgatca |
| 165481 | aaaataaaga accacttttt acaaagtgtt tgtgtcacac cagcttaaat gcttccttct |
| 165541 | gctatggcga tgtagctcag tggaacagaa ctttcacagg cagcttggcc cagacaagat |
| 165601 | gacccaggct gttcagggta tcctggatgc tccagccccc acccatgaaa cagcaagcag |
| 165661 | ccataccaca tcgatagaga actgacaaga acatttgaca gagaggtgtc caacgtgaga |
| 165721 | gcagcaatct gttttcccag gaccttctga cagactgcca aaaaaaaaag atgttaatta |
| 165781 | tcccaacctc agaaattctg ctgaaaagag aacccataaa atcaggcccc ccaaaaaaac |
| 165841 | aaaatgaaaa attgtatagc aaaactcatc tactggagat gatcactttc aaaataaatt |
| 165901 | tctggaaacc aatacaaaag aaatctacac agtaggattc caaattggac ttgtcctaat |
| 165961 | atgctagtaa ctcacttcca acagcgtcaa tccagacaag aggaacaaga aactgaactt |
| 166021 | gctaaaaatg caaagtgagg ttggccaaca ctggtgctcc acaccaactg gatcgagaat |
| 166081 | gtcaaacaaa gtggcacact gggcgaaaat taatttcatc ttaaatactt tctactttca |
| 166141 | taacatgcaa tgttattagt tacaagcatg ctgggggaaa atgtatattt ctaaatataa |
| 166201 | tctttgtgtg aatgatacct gtcttatgta aacagaaaag actttctgtg gactggaaca |
| 166261 | actgtattaa agatcgaagc tgcaaaaaca tgacattcaa cagcccctct accatcatct |
| 166321 | ccagggaRgt ccttttaaac agaccaggtg ttggcagaga Ytcggaacca aaatttaaag |
| 166381 | cctttttcaa gatataagag gagaaaagag atttcatcct ccctgataaa ttcaagaagc |
| 166441 | acttctccaa aagttaatta gccaaaatcg tcacagggct tgctatgctc tctagaagaa |
| 166501 | aatcaatgac tagttctccc taacccgtag aaagcaattt aaacagcaga ggacaaatgt |
| 166561 | cctttccaac atggtatttc tgccaaaaaa aaaaaaaaaa atcccacaat gatgggatcc |
| 166621 | caggagtgac atttacatta aagttgaagg cgtcagatgg agcagggaaa ggctggcagc |
| 166681 | aaatgactca tctgtggagt ggtgaccagt gtaccctggg ttggatcaca catgggacag |
| 166741 | tcccagccat cacctgcatg caccaaactg agggggctgg atccccaagg gatgattgat |
| 166801 | gtcttaggct agaggaagct ggcacaaggc agggtagaat gatgttctta tcaacaactg |
| 166861 | acttcaggct actcaaggct gcaatcagaa ataggaaagg caagggaaac aaattgctgg |
| 166921 | gctccccagg ctccccagac agcattctgg ttgcagcagt gctggctgcc tagttctagg |
| 166981 | gggctttgtg agtgatggag gtMcttctgt gatcccatct gaattgagaa gccagMccag |
| 167041 | ctctgagagc tgagtgtttc tacatgagat ttccagcaac cctccttgag cctatctgtg |
| 167101 | gagatggact ccagggggcc aaagaagact cagaatggga aagatgagac ttctccaaag |
| 167161 | tgactgccaa ccaggctcca aatgtgattt ttcagagccc atcaatcaca acccctctaa |
| 167221 | gaccctctgc tcctcttcct ccaacctcct gctctcaaga ccacacctgc ttgctccgat |
| 167281 | ttctcagatc actctcaggt ccagccagac acctgggctc ctccctttca ctgtctctct |
| 167341 | ggttctgtgc tctgacctga ggacaggtgt cctgccatac cagaaatgtg agtccaatag |
| 167401 | caagtttctc atcttctgcc agaagcctgc tcccgcccac tcaagtctca atcctcctag |
| 167461 | gggcgggggt gggggtgggg gccgaggcta tgctgccctc cctctcccta gcaacttggc |
| 167521 | cttgactctc cccttgcctt caaaccatct ggaactgagt tatttcattt atcaggaagt |
| 167581 | cgtcctttct ctccactccc cctgtgtggc cccaagtccc ctttgctgtg taaacaggac |
| 167641 | cttggcccat cactactatc tgtgcacaga agcagagata agcagagctg aagagaagcc |
| 167701 | ctggcctgtg gcaggattgt gggggctctc cttcccccag aggcgcctct tcctcactRt |
| 167761 | gggcaccaag ttttcctgct gtctcactca gctgttcaca ccgccagagc cgcccacaaa |
| 167821 | cacacacgRa agctcatcta caccaatatg ctaacttcct gctatttgca tttaaacatg |
| 167881 | tttaactcaa actgttcatg gaaagctccg tctcccattt agtcttttag aataactggc |
| 167941 | acacaggggg caatatctgg caaggtgcgg gaaagttttt attttccttg aagtggtcct |
| 168001 | gttcctaatg catctcttga aagaagacca agtttagtgt ttgttacaga ctgaattgtg |
| 168061 | tcgttcctcc tcacagaaaa aatccacatg ttgaaggcct aaccctcaat gtgatggtgt |
| 168121 | ttggagatgg agcatttggg aggtgatatc atttgatatt tacccctgcc caaatctcat |
| 168181 | gttgaattgt aatccccaac tctgggggtg gggcctagtg ggaggtgttt agatcatgtg |
| 168241 | ggcacatccc tcatggcttg gtgctgtctt cgtgatagca agtgagttct tgcaagatct |
| 168301 | ggtcatttaa aagtgtatgg tgcctccctg ctgactctct ctctcttgct cctgtttttR |
| 168361 | tcatgtgaca tgcctcctcc ccctttgcct tctgccatga ttgtaagttt cctgagggct |
| 168421 | ccccagaagc caagcagatg ccagcactgt gcttcctgta cagcctgcag aacgatgagc |
| 168481 | caattaaacc tgttctttat aaattaccaa atctcaggta tttctttata gcaatacaag |
| 168541 | aatggcctaa tccaggaggt aatcaggtca ggaggatgga gMcctcatga tgggattagg |
| 168601 | gcccatataa ggcagagaca tgagcaagct ctctcactct ctcaatctct gccatgagag |
| 168661 | gacacaaaaa ggcagctgta tgtgagctat aaggaggact cttacctgga atctgaccct |
| 168721 | gctagcaccc tcatcttgaa cttcccagcc tccagaactg tgacagacaa ttatttaagc |
| 168781 | caggggtccc aaacccctgg gccacagagc agtaccagtc catgcctgtt aggaactggg |
| 168841 | tggcacagca ggaggtgagc tgtggggtac aagtgagcat tgctgcctga gctctgcctc |
| 168901 | ctgtcagatc aatggcagca ctagagtctc atagaagcgc aaaccctact gtgaactgca |
| 168961 | catatgaggg atctaggttg cccactcctt atgagaatct aatgcttgat gatctgtcac |
| 169021 | tgtctcccat caaccccaga tgagactgtc tagttgcagg aaaacaagct cagagctccc |
| 169081 | actgattcta cattatagtg agttgtaaaa ttatttcatt acatattaca atgtaataat |
| 169141 | aacagaaata aggtgcacaa taaatgtaat gcacttgaat catcccaaaa ccatcccctc |
| 169201 | cccgacccca gtcggtggaa aaatgatctt ccatgaaacc agtccctggt gccagaaagg |
| 169261 | ttggggactt ccggtttaag ctacccagtc tatgccattt taatacagca gctggacatg |
| 169321 | actgaggaag tgtttctgga tgtttcccaa aaaagggcta aggaggacag gtgagagaga |
| 169381 | ttccaatccc ctttccttcc acattttttc ttcttctaac aaagacaaca ataataacac |
| 169441 | tataatatca atttcaactc aWttcaatgt ttagtatttg tcttctctac tagactataa |
| 169501 | actccatgag ggcagaggcc acatctgcag agccactgca cacagacact caggctgaac |
| 169561 | aactccagca agtaccattc acatcagcgt caatatgaag gccaccccct ggagtggtgg |
| 169621 | agcccagcac tcctgcatac ctgcctggcc ttcttgctga tttacccgca gtgttcagca |
| 169681 | cagagcctgg cacataacag gtgcccagta agtctccatt gaaaagcatt tgacaactca |
| 169741 | cattccactc acctagggca ggcatttaaa ttacagcagg aaggctaatg tggcatgctt |
| 169801 | gtgcacagtc tgcagagtct gtgactggca atttaaactg gatggctgtc cacttgcagc |
| 169861 | ttcagaaaaa cattgagaga atgtctgcat tcactctcct gtagtcctca tcacccagca |
| 169921 | cctgggacat tgcagcactg ggtggcctaa ctgcaagcgc tatagcccta ctcttaccca |
| 169981 | tcctgacaaa aggaattctc cctgaaactc agtgtttagt ctttcaaact cctcagtgta |
| 170041 | gaacccaaga tggctcccaa tggtgcccag gttcctctgc tgggctgggg atgcacccgg |
| 170101 | aaaaactatg tattggggta aagtggaaaa tcaagacttg gaattggtca tgtctgagtc |
| 170161 | cagctccacc tcttaatagc tacacaaccc tcagcaagtt acttaccctc cctgggccta |
| 170221 | aattctacta ctcacaatat gggtaatata atctctactt cagacctgag aaaatttaat |
| 170281 | aagctagaac ctctggcaca cagacataaa agtctgMaat caatagccat tcctttctcc |
| 170341 | ttccccaaag tgcagtactt gctgccactt tgtggcatga ccctctgcct gagcaggctg |
| 170401 | atacccacca tgtgatgcaa cactcactat tgcggtgtcc ttgaggctgt cggggaccca |
| 170461 | ctcaaacctc tgtcccagga cagaaggcct ctgggtcacc aaggctgagc ctcaggcatc |
| 170521 | caggagagca gggcagctga tgcatggtga ccccagcaat ccacccaagg actgccctaa |
| 170581 | gcacagagac cacatctggc ttgtttactg ctctctccca aagcctagta catgctgggt |
| 170641 | catgcctgcc acattgggta aatgatgaRt gaatgagtga atgaatgaat cctcctctga |
| 170701 | cccccctgca tatatgaaac agacagccct acccctgaat ctcaacaaaa cactttactt |
| 170761 | atatccttcc tcccacctgg aatttttgta ttctgtcctt gtcctggctg atcttagcct |
| 170821 | agtcaagagc tggctgaaaa cttgcttctt ccaggaaggt tcacaggtta tctctaccag |
| 170881 | tagggtttgc tctctggctt ctctgagcta ccttatctgt cttactttat ttttgatggt |
| 170941 | ctttgccacc ttccttatct ctgctcactt aattagattg gaagtatctt aaagacagga |
| 171001 | actgagagct atgtgcttgc atgctaccca accatgcaca gtactgggaa tgtagaagca |
| 171061 | tgtgatagat acattgcttg actgaactat catgtcatcc tcagcaaact aatgaaagct |
| 171121 | tcacagttga gaggtgcttg gaacgttcta actttctcca tgggaacatg aggtgagcct |
| 171181 | gaagttatct ggctacttct ccagagccca ggattaggca tctgaaaata gatatcctcc |
| 171241 | aagttcaaat cctattgagt tttttcccat atccttaaaa aaaggtcact tcagYtcctg |
| 171301 | agattttcat ttctccatgt gcaaattcat gctaccctgc aaggtgccac agaactgaca |
| 171361 | ttctccttct ctgaacttta caagaagctc actgNttctc aatgctgctg acatcagacc |
| 171421 | tatgccagga Raaccagctc cttctctgca tgggtagaaa tgcccttctt gtgcttagtg |
| 171481 | agaatgccat caccacatgc tccaaaagaa tgctctcaaa ctcaggattg tgaaagaatc |
| 171541 | gctaaggggt gggaagctgt gcaattctga ttctagaggc ttgggatgtg atccagacat |
| 171601 | cagctcccca gtgatcctga gaaatWtgag actagctgat ctaaagatag ctccccaacc |
| 171661 | tctaaaacaa gggcatgaat gagaggggac ttgtgttgct cctcagccac catgtttgaa |
| 171721 | tggcagagct ggacRgccca gttagcagac tttgtttcat tcagctggtt ttgtttctgt |
| 171781 | gggaaaactt ctttcaggca gctggcatgg attaccctgg cagcactaac ctgcagcaga |
| 171841 | ctaatcccac aaaagtcctc atccctaaca aaattgccaa aagccctggt caaggcaaat |
| 171901 | gtatgaagat ctgacatcct tagtcattct gtgggtggct ctgaccttca aaacatctca |
| 171961 | ggaagggcac aYtggggaca aaggtatccc ttgggaaagt tatgtccgac aaacagcttg |
| 172021 | aaccctaaaa catccatcat gacttcgatg tggctcagtc tgatgttcag gcccagtaga |
| 172081 | cccaataaat cccttttcca gcatctaccc tggtgaagac ttttcaggga cttattatgc |
| 172141 | aaagctgcct aagcaagaca gaatttatat tgaacctggc catgatttct gagccgccac |
| 172201 | tgggtgttct gatgacatac acatctcgag gttgaagtca aggagctgcc aaggacaWga |
| 172261 | gactggtggc aggataaaag cagtgtgatt ccttccagta aataatggtg gggtggcggg |
| 172321 | ggaggtggga aggggacgct ccggggagga agaggaccgt agcttttctt gtacccaagg |
| 172381 | tttgcctttc aaatggcatc cagacgtgac caaataaatc tctcctctgg aggctaaaga |
| 172441 | atgtgtaSaa tgcaacaaaa gtgaggtggt ggagaagcct gctttatcta gggtgaatca |
| 172501 | tacctagaaa gaaaacatgc atccgtcaca gaaatttcat tgattgagaa aatattttta |
| 172561 | gtttcctggt ccacagactg agtccttcct gaaactctca ctatgagctg ggcactcgga |
| 172621 | tgttgcctcc tgagtgtgat cgcctataga agcagactat tgattagaga aaattcttag |
| 172681 | acaaacagat ccccagccct catctttgcc actggaatgc aaagggtgct gccctcccca |
| 172741 | gtaaagggaa tatcggtctg gtatttaaca cggccacaaa gaacatgttg ttctatagtc |
| 172801 | aattctctat acagcagccc aagtgatttt tttttttttt tttgaaacaa ggttttgctc |
| 172861 | tgttgcccag gctggattgc agtggtgcag tggaggagtg tagcctccac ctcccagMtt |
| 172921 | caagcaatcc tcccacctca gcctccagag tagctgggac tacagacaca cgccaccaca |
| 172981 | cttggctaat ttttgtattt ttttatagag gcagggtttt gccatgttgc ccagcctggt |
| 173041 | ctggaactcc ggggctcaag cagtcagccc gtctcagtct cccaaagacc tgggattaca |
| 173101 | ggtgtgatcc actataacca gccccagtga tcttttaaaa acagaaatca gggctgggca |
| 173161 | cggtggccca cgcctgtaat cccagcactt tgggaggctg aggtgggtgg gtcacgaggt |
| 173221 | caggagttcg agatcagcct ggccaacaca gtgaaatccc gtctctacta aaaatacaaa |
| 173281 | aattagccag gtgtaatggc ggacacctgc agtcccagct acatgggagg ctgaggcggg |
| 173341 | agaatcactt gaacctggaa gacagaggtt gcagtgagcc gagattgcac cactgcactc |
| 173401 | cagcctgggc gacaRagcga gactccatct caaagaaaaa aaaaaagtgg aaatcaggcc |
| 173461 | atggcaKtct ctcctactaa cacctccaat gtcttctcac tgcactcaga gcaaaatcca |
| 173521 | aactccttcc catggctaca ggtccccagg ctggctgggc tcacctttcc tgcttcatcc |
| 173581 | tgtcccctgc ctccttcccc gctgggctcc atcctcactg cactcctttc tgttcctctt |
| 173641 | ctatactaag ggtatctcac ctcaggacct ttgcatatgc catctcctct acctggYatg |
| 173701 | acatgcttct cccagatctt cttgtagctg actgctcctc tgagatgtct tcttaggttc |
| 173761 | accctatctg gaagtagccc accccacccc aaccctgccc taccctttgc tatctcatat |
| 173821 | actttatttt tttcataaca ccactatcaa agtttttctc tttgcctact gatcatctat |
| 173881 | acacaactaa attgcaagtg ccacgagagc agcgtccttg tctcatcaga ttcccagccc |
| 173941 | ccaacgcaag acttggcaca taggaaatgc tcgacacata tcactcaaca Raaaaatgaa |
| 174001 | agctctaggc ccacctgcaa ctgacagctt gaatgatacc gaatcctggc cttgtagaag |
| 174061 | gctggaagtc agtgctggca ggagaccccc Wgcacaggga ataagaatca cacagctgcc |
| 174121 | accacctgat tcctggccct ttctgcccaa cacaacataa atgacagcag gtcagtggca |
| 174181 | cagtccacag tccacactca aatcttgatc atggccaccc acatacccca aacctggaga |
| 174241 | gagactgggg tgaggtagat aaacgatgcc attcaagaca tcagacattg ctttctatgc |
| 174301 | ccaaaaggca cattccaggc ctgatattgg caccggaaac tagcagtgcc agaagatggt |
| 174361 | ctccaacata cccatcccat gtccctcaaa gggcctctct ctggactaac ctcttctgca |
| 174421 | tggtctttcc tgcagagctg atccgtgtcc tcctgcagag ccctgggccc acgggtggtg |
| 174481 | ccaggggcag cggcaggatg ggggtggggc tgcttgacag gaaatcaatg ggaactgaag |
| 174541 | gtcatccgga gggcgcagtc tccccatcac Kcacctgctg gcatgggatt tgtggtcctg |
| 174601 | acctcgcttg ggggcctctg ccctcctgtc agagttggtt aaaatcagcc tgcagaagca |
| 174661 | aatgtcctgc acaccaaatg atctccaggc agcccttcta agggacatcc agccaaacaa |
| 174721 | agaagcactg agtctcattt cacccgccgc aaagttgaga gtcaccccaa tctctcttgg |
| 174781 | cagcctccta ggaaaactgt ctcagacctg ccatttttgg aggcacatac ttgtaaatag |
| 174841 | aacaaagcca gactgaccaa gaggctggtt atgtccccaa acttaagttg cctggtggtg |
| 174901 | gaggaagtaa aaatacactg ggaaccaaga tgactgaaac tgaaatcttg agtgcaccct |
| 174961 | agaaatgcct aaatctgccc tcaacacctc tacatgccag gataacgaca aaggatacca |
| 175021 | aaacctctgt actgccggag ggaaatctca ttttcttctc atcccaaaaa atactgggga |
| 175081 | gcttggctga atctagaatc tgattttcct tcccccacta tacttatttt cccaggttcc |
| 175141 | cattctccct tgcttcccag acctttcccc tctaccatct tctttcccaa ttcgtccacc |
| 175201 | tcatcaagag tcatttatca tacaggccag gagcaaagcg gtcagcctaa gtctccaatt |
| 175261 | caaaagttga aaatgggcca ggcacagggg ctcacgcctg taatcctaac actttgggag |
| 175321 | gccaaggtgg gaagactgct tgagaccagg agttcaagca ccctggccaa cttggtgaaa |
| 175381 | aaccgtctct actaaaacta caaaaattag ccaggcttgg tagcaggtgc ctgtagttcc |
| 175441 | agctacttag gaggctgagg caggagaatg gcttgaaccc agaaggcgga ggttgcagtg |
| 175501 | agctgagatc gcaccattac actccagcct gggcaacaag agcaaaacac cgcctcaaaa |
| 175561 | aaaaaaaagt tgaaaatgaa tatggtatga aaataaagaa aaggccaggc acagcagctc |
| 175621 | atgcctggaa tcccagcact ttgggaggcc aaggcaggag aatcacttga ggccaggagt |
| 175681 | tgaataccag cctgggcaac atagtgagat ccccgtctct acaaaaaaat aaaaaataaa |
| 175741 | aaaatgtttt aatgaagaag agacaccctg gacatattta cagaaggata acaggatgct |
| 175801 | ggttatagta gcaatagtgg catagctaat atttattgag catttactat gtgtgccagg |
| 175861 | cactgttcta agcactttac atgtattatc cccatacaac ttgtaattgt attattccca |
| 175921 | tacaactcca tgattttata cccaccccct cacacacccc actttacaga ttggcaaact |
| 175981 | gaggcacaga gagtcttacg tgatttgcta aggatctatg tggccttgtt aatacattac |
| 176041 | agtcttgcca gaaccttact cttggccact gcactgtcag cctcttacaa gggcgccatt |
| 176101 | accccctcac aatgagtttt gcctccagaa ctagaataac ctgaacactt cctttcttgg |
| 176161 | aggcactaga acacttcctt tcttggaggt ttctatcagc atcaccaact tgctagctaa |
| 176221 | tcttagctat ttcttttctt tgtgcctcag tatcccaaac tgccaaatag cccagaaata |
| 176281 | aaccttctta tcacagagcc acctctcagt aactcaaaaa aagttaatat ggggcttcct |
| 176341 | gaaaatatgc catcagagaa atacaaaatg tcattatgca gtttagaaac caaatccatc |
| 176401 | ccaaatgtga gcaaggggcg atggacttga agaggccctg tggttctgaa catcaactaa |
| 176461 | ggcagaaaaa gagaaatgcc agaaagtcat aaactaaagg gtcagattaa agaatcccag |
| 176521 | aatgttagac actgtctagt ctggtgcctg gttttattgc aaaggaaatc aataaccagg |
| 176581 | ccaggtgcag tggctcacgc ctgtaatgcc aacattttgg gaagccgagg cgggtggatc |
| 176641 | acctgaggtc aggagttcga gactagcctg gccaacttgg tgaaactcca tttctactaa |
| 176701 | aaatacaaaa aattagccag gcatggtggt gcatgcctgt agtcccagct actcaggagg |
| 176761 | ctgaggcagg agaatcgctt aaacccggga agcggaggtt gcagtgagcc gagattgtgc |
| 176821 | cattgcactc caacctgggc aacagagcga gactccacct aaaaaaaaaa aagaaaaaga |
| 176881 | aaatcaataa ccacagaggt gaggtgacgt cctcgggaca taccgaggca gagctgggat |
| 176941 | cagatcctca gtccagactc ctagttcagg gattcctcca tcgtttatgg caagggtcaa |
| 177001 | caaactacag cccctcggcc aaacatagcc caggactaat ttttgtaaat aaagttttat |
| 177061 | gggaggcaag ccatgcccat ccgtttacat attgtctgtg gcagcttttg tgctgaaatg |
| 177121 | acagagtcga gtagttccaa cagagaccat gtggcctgca aagcctaaaa tatttaccat |
| 177181 | ctggaccttt acagaaaaag tttgccgact cctggttttt aacataagcc ttgaataatg |
| 177241 | aaaagcctcc accctcagaa tacaaaataa tagaatagga aacctaagag tcacacagct |
| 177301 | taagcagata gttttacaat tatctcagct tggtacaacc taataaaaac ccataaatac |
| 177361 | agttaataat aaggaaaaca agcactagct caccctacct atttaataat tattgttaac |
| 177421 | tacagcaagc aaaactcata gctgaaaatt cgtttgtttg cttttttcta gcctcccttg |
| 177481 | aaagccctgt ttcatctagg aaatgctgtt ggccaatggg cgaaaggaca catcctagag |
| 177541 | gcgcacagcc ctaatttcct cctgttgagt tgcaggctgc tgctgaggca gctgaagact |
| 177601 | tgagagcatt gccctctctc cagagcggac tcagagagag ttgattttct ctgtcaaagg |
| 177661 | cgatggcgcc aaaaccctcc tgtgcccctc actccagata gaaccctcag gaacagccag |
| 177721 | gcatggaaga gactgtcttt atggcttcct ctccctactg ggcctccatg ccctgcttaY |
| 177781 | acccctttcc tccccagaaa gtgatcagga tatgcacacg gcccacagaa gcccagccca |
| 177841 | cagctatgcc atcattctat catcccatgg aatagggaaa agcgggtctg cccctgttag |
| 177901 | gctgatgtgg gaatcaagag acctggattc cagattggag ttggaactta gtcactttgg |
| 177961 | gtttttggca aatctctcaa gccctctggg cccaggtctc ccacgtcaaa tggagagatc |
| 178021 | gtcttcacat attccaaatg atggaacacg ggcagagcca aattaagggg tggggcaggc |
| 178081 | caaccacgtg gcacccgggc tccagtctct aatcacacta agtcattaSc aggaacatag |
| 178141 | aaaggtttac tagtgtcact caggcagaga actttgagaa ataggacctg gctttcatgg |
| 178201 | aatagtccag ttcgcatcaa ggtcccactg taatgattat tacacttttc cctctcaaaa |
| 178261 | gcgcccttgc ttggctgata aatgataaag tcaccctact aataagctgt ttgtggtggg |
| 178321 | agtgaggtga gacacgtggc ccaaggcacg gacagtaatg gcaatacatg ggcttctaag |
| 178381 | gaagacaagt tcagtttggt caagaggtgg ggtgcaggtc cggggccaga actgagctgc |
| 178441 | taaggacaag agaggagagg ctgcctctgg tggcccggct tctccatttg ccatgcacac |
| 178501 | agcctttgcc tcttctttta tttgtcaaat atttagagaa tattcgccaa agtgtgagct |
| 178561 | ccccaggagg cccacaggtg ctggagagaa gagagggctt tggagagtac tgtaaggtga |
| 178621 | aagaaaccta ccaacaggtg cctaactgcc tggccatgct aagcgcctga gccctgggac |
| 178681 | agggtatgat ccMagccctt cacagtgcct gaccctgacg ggcatgcact tggtaagtgt |
| 178741 | tcattctggg gacctggaac aagcgaagct caagccctcc ttctgttctt ggaaaactcc |
| 178801 | tcctccccat ggcagctgca tctcgaccac caatcctact ggaccacagg atgcaggagg |
| 178861 | actacactgt gtcctggatc tcgtcattac aagagcagga ggtgagcact gagaaagatc |
| 178921 | cagaagaact catgcaagca gaggctgtgg tctgtgggga ggagggccgg gcagggcagc |
| 178981 | ggaccagagg aagaagcaac aaagaaggaa ggggaagagg aggaggagga ggaggggggc |
| 179041 | cgcagagtca ctgtgggtgg agggagagca ccgccagcac atgccccaca gccatcaata |
| 179101 | tctcttcgga ggacagatta tctggggaat ttatgctctc taataattac cttgcagagg |
| 179161 | gttccactta tcttaccaga ggaatctgcc agttgccaga cacacaggat tctagtcaat |
| 179221 | tccatccaca ctgccctccc cctctgccct cctccccacc tccctggctc ctgacttctg |
| 179281 | acctctgaat tctaaccttt ctttgcctct ccagtctagg gggaRgttga tggtggaagg |
| 179341 | gtcacaacaa cttttaacag atgtaaaggc caacaaaggg gttggtgggt tgtcccttat |
| 179401 | cagtagatat gagagttaac gtcccaaagt tgaggccagg cctgaggaag tataggctct |
| 179461 | tgccaaagac agtgtgtgaa gggccaagca atgctttggg gcagactgac Wtgattattt |
| 179521 | ttgtcttcac ctgatgccat cagacctggt taggattaat tttatcttct agtcttcaca |
| 179581 | tccctcctca aagccttcct atccccaStc cagccctcca gtgaggcctc cctccaataa |
| 179641 | tggYgtggct acttattcga aaaccatcac gtgttgcttt gggttgctgt ggtttcttta |
| 179701 | aacgcacgtt gcagtcctgg ctgtgagctc ctggagaaca tgggtactgc atccgtactg |
| 179761 | aatcaggact acctgagcaa cagtaacaac atccagatgg ccctgacRgt gggcgggaca |
| 179821 | gtgccctccc tgcttagaaa caggaaccac atttcatctc cacaacaacc ctatgaaaga |
| 179881 | gatgctacta ctacccctca ttatacagct gaggacctgg gagctcagac agcttcagtc |
| 179941 | acaggcccac ggtcacttgg tcagtggcca agtcaggatt tgaaccaggt ggtcttgttt |
| 180001 | cagagtctgg gctcttaacc actccacacc acgctgcctc Ygtaggagag gtttgctgac |
| 180061 | tgattcggac attttgtgtt gtatctatcc gcttatttaa gtcatccttt tctaaaataa |
| 180121 | taattttttt tgagacatgg tctcgctctg tcactcaggc tgtgcaggag ctctgtcact |
| 180181 | caggagtgca gtggcatggc tcactgcagc ctcgatcttc agagctcaag caattcacct |
| 180241 | acttagctac cccagtagct aggactacag gcatgtgcca ccattcccag ctaatttttt |
| 180301 | tttctttttt taagagagat cgtgtctcac tttgttgccc aggctggtct caaactcctg |
| 180361 | gactcaagtg atccacctgc ctcagcttcc caaagtgctg ctgggattac aggcatgagc |
| 180421 | ctgggtgcac ctgggctggt gctcctcctc cttaggactc cactcactgc tccctcctca |
| 180481 | gggggcctcc cccaaactcc ttagctctgg cactcattct ttttccatca ctctggcacg |
| 180541 | atgccaacta tcaaaaatta ttttataggc taggtgtggg tggctcatgc ctgtaatccc |
| 180601 | agcactttgg gaggccaagg caggcagatt gcttgagccc aggagttcaa gaccagcctg |
| 180661 | ggcaacatag tgagacctct tccctactta aaattaatta attggccagg cacaatggct |
| 180721 | catgcctgta atcctagcac tttgggaggc cgagatggga ggactgcttg agaccaggaa |
| 180781 | ctcgagacca gcctggtcaa caaagcgaga ccccatctct taaaaaataa taataaataa |
| 180841 | aaaataatta attaatgtat atgtttattt actacctgct tttctcaata gatataaaag |
| 180901 | cttcctggga gaaagactgg tctaatttgt tcacccctct atcaccagtg cctaaagctg |
| 180961 | gtccctagca tatagtaggc actatataaa tatgagttgg atgaatgaat tgtctgatgt |
| 181021 | ccccactgag cattccataa tacatgatgg ctacttaatt ggacacttta ttttatgcaa |
| 181081 | actcatctat ctgttcttta tttattagac tttacagtgt accaggcacc ttgctgagta |
| 181141 | ggttaggatt tcactgtacc ctgatatgag gtagagaata ttgtctttat cattctcatg |
| 181201 | ttacagatga ggaaactgag actcagaggt tacacagctt gcccaagacc atacagaaat |
| 181261 | agggtaagac ttaaccccca ttctgtcagt ccccctgagt ccatgatatt aagcattctg |
| 181321 | cctcattgcc tctggctggg tcccctcagt aacatgattc tctttacccc tgaacctcta |
| 181381 | aaatgtcccc agccttactt ctcccccctc aagagccccR tcRcctcttt ctctggcctc |
| 181441 | tgtgtgctgg ctctcctcca ttggtccctc cagattctct ctctgccatt cttttccctg |
| 181501 | ctgggtgctg cggaagtctg tcctgtatgg tcagccattt gccctcttcc ctctgctcct |
| 181561 | ggtggtctgg gtcaatggca ggtgccgaga gagggaagag agacagggga tttcctctcc |
| 181621 | ccgccccttc ctgctgtgga gtgtctgcat ctgcccactg aaggtcacag ctcctgtggg |
| 181681 | ccagccctct tcatacagct acctctgcaa tctggtgacc ttccccactc ccccgaccta |
| 181741 | gccctaggct gctgcactag ccccccacca tgtgagtccc tgcccgcagt tttgaaacta |
| 181801 | gacatgttac taaacactcc tcagtttact tcgtgtggag ctgctgcttt gctggaaccc |
| 181861 | tgcctctcgg acatatgagt gctacatggc ctctgcccct cccccaggtc ccccaccact |
| 181921 | gccgctgccc cYtggtcact gcagctgcct caccaggccc ttctttgctg ccagggtttg |
| 181981 | ttctctgtct cYaagaccta taaataaatg ttgatttgct ggcttgaagg tttgccctgg |
| 182041 | cagccccacc agggagagca gtgccctggg gagcccagca agcccacagc aagctgtcct |
| 182101 | cccaagacta ggagaaagca gagagcatcg ggtccaatgc aggggtcccc tatgctcgga |
| 182161 | gcttccccct gtgcggggtg ttaagggacc cctcagatga tgtcctggtg cctcaaacaa |
| 182221 | agccgccatc acagcttcaa aatcacaRta tcatttgttg agcaactact atgtggcagg |
| 182281 | ttctgggttg ccatttgcat acattcactt tcatgttaaa agacgagaaa aacaRgttca |
| 182341 | cagaggttaa ggaatttaac caaaacctca aaactactaa gtggatgaac tggaattaga |
| 182401 | tcccaggtca gcccaaatct ataaggcctt aattccccac cagtacatgc tgcctcatga |
| 182461 | ccgatccctt tgaatgacag gaggcccaca gactcgaacc tagtccaggc aggcagctca |
| 182521 | gagatgccag ccacccatgg ctgggaggag atcaggtgga gcaggatgac agttaggata |
| 182581 | aagggcacag agggctccat gctgcctaga gtcactttgt ccaaggacca tggcacagtt |
| 182641 | tggccatagt gactgctgag cagccagggg gctggaacat gccccccacc cccgccccca |
| 182701 | accccgaccc actgctcctt cctgcagagc cagccaggag ccatcactcg tccttgggca |
| 182761 | cacaccagca tttcaagaaa acaagcctgc aagcacagac ccctctgagt caggcccagg |
| 182821 | actgtcacga ggagactctg aaggggaggg gagggacagg gtgacacaaa gagggggaag |
| 182881 | gcctcctgcc gctggacctg gagccgactg tgcttcccta agtcctttct gcagccaaaa |
| 182941 | ggcacattcc ctgagaacac cgctcaagat ccaaaggctt tcgcttttta ttttttagga |
| 183001 | agcaatggca aaaaaagaaa agaaaaaaag aaagaaaaga aaagaaaaag aaatccacat |
| 183061 | ttctagtgtt gggagcgatt cttggaggca aacacactct cttgcacaca cacaccccac |
| 183121 | agcgcttccc ccacacagcc caaaatagtc cacaccatgg actcctggag aacccggctc |
| 183181 | agcacagcct cccgaccttg gtccacatga ctgctggcca gctctcagtc cccagggtaa |
| 183241 | caaagtactc caaggacccg cagtagcagc gcctgctcct acagaccatt tcacccctcc |
| 183301 | cagcgaagtc ccatctgctg tgtctcgatg gagccaggtc cagttagcag aggccacttc |
| 183361 | caatgaccca gtgtggccca aatctccaga gctgacctga acctctctcc ctcaacccct |
| 183421 | ccaaatctta gattgctgct gctgcttctc caggggaatg gctcccacaa cattttatag |
| 183481 | actgcaagga aaatatccag aactgagacc gttcttcaaa ataataattt aaaaaaaaaa |
| 183541 | aacactgctg aaacacagaa ctgggtgtta tcttaaaatc tctgcacaaa tagaatttga |
| 183601 | atgctctcac tccaatcaaa atcattctcc gtccctccct ttaaacactg aaagttcaat |
| 183661 | tccccctaaa gacaggattg caaacatgat taggtccaat taatttatgg agaaactcag |
| 183721 | aatgaaagga gagaaatcaa tagaactgcc ttgttctgat cactaccgca aatgaagtaa |
| 183781 | taatttgaag gagatctgga aagaaaggaa accaaaagtg tcacaccgca gccattggcc |
| 183841 | atttaacaag gcagcattgc cccatgtgga ctggctgggt ggagaagacc actcaagctt |
| 183901 | acaagacact cctggggaca gagggagggg agagacagct gaggtcactt gctttctctt |
| 183961 | cattgtacag catgaggctc actggtggta aattctcctc tttctcacaa aagcaagacg |
| 184021 | taaaagaaag taaaaggcaa aaagcatcca cacaagctgc cagtctaata aggacactgg |
| 184081 | taatctccag gcaggccaag gccatatggg ctccccctgc agcttcatgc taagtttatc |
| 184141 | tccatcctca cactctacct ccatctctct tccatatcca ggaaggaaaa aaggaaatta |
| 184201 | acaagccggc atccaacatc catcctgaat caccagctgg gccatgtaga gcatccaaag |
| 184261 | atggggtRgg gaagccccag gggagagaga gacattgaca gatcaccgtc cccacctctc |
| 184321 | acttgcctgc agctcccttg catagatgat gctggcaaat tcaacRtgat tttcattgga |
| 184381 | cctgcccagc agtccagatg tgggagccta ataacaaata acaatgccag ctccatgtga |
| 184441 | tccttgcacc ttcaagagaa aatgccttgc tggccagctg ggttctctgg ccaccctcca |
| 184501 | gcccaccttg ctgcttctgg atggaggttc gtgaagctgc tcctggccta tccaaatggc |
| 184561 | cagtaaacat taacattcat ccagtcaggc aacatccatg gagtgcctat tccatgccat |
| 184621 | gctgccccat gtgggaggca gcaaagcgct tcagtgaaag gctggggcac tggcatgagg |
| 184681 | acaccaggtt tgagtcccag ctccttgctt acttgctgtc tgagctgttg aactctgtga |
| 184741 | gcttcactac cctcagctgc aaaatgggta tgataataag acccttctca tggcttcact |
| 184801 | gacaagaaca aatgaggtga cctcactatg ggtgatcagt aaatattagt ggttRttacc |
| 184861 | acaatgtggg gtcctaccct ccactcattg ccaccaccag tgccagaaaa agccagctca |
| 184921 | tgagttaact cccaggcagt attatccact ccaggggcct ctctgtccac acagatgagc |
| 184981 | agaagcagaa tgcatgaaaa aataaaatag ctcaatgcca ttgctacaga ttgccccacc |
| 185041 | accaccatct caccagtcag cttcaagcca gcggggtcag cagtcagaag cacagccaag |
| 185101 | agtgtgggga tgattccgtg acaaaccatc cccagcagca gaaacctatc tcacagcaca |
| 185161 | catcctccca gaagtgggtc aagggcatcc attgcctttg tttaaaggca ctgggtcaca |
| 185221 | aaggaggagg agccttagca ggttggaact ttgggatatt ccacaggaca ggtagaaggg |
| 185281 | tgctgtgttg gttttctatt gctgctgaga caaattacta caaactttgt agtttatgaa |
| 185341 | aacacaaatg tacgatttta cggttctaga ggtcagaaat ccaaaacgag tcctaaggct |
| 185401 | aaaaatccag gcatcagcag ggctgtattc cttttggaag ctctaaggga gcagtttctt |
| 185461 | ccttgacttt tccagtttcc agcagctggt ggcctcttcc tcccatgtgt cacttctacc |
| 185521 | tccacttcca aYgtcatatc tccagctctg actctgaccg tcctgtctcc ctcttgcaag |
| 185581 | gacccttgtg attacactgg actcacccac ataagccagg ataatctcct caYctcaaag |
| 185641 | tccttaactt aatcacacct gcaaaatccc tcataccatg taaggtaaca tattcatagg |
| 185701 | ttccaaggat taggacRtgg acatctttgg gggtaaggtc acgcttctat ctaccaaagg |
| 185761 | cgtgttctgc tgctgctgca aggtacactt gaataccgtc aaagcaatgg tgaagaattg |
| 185821 | aaagaaacgc agttatgtcc taagcccagg aatattgaag ctgtcctctt tttgacaaaa |
| 185881 | aaaaaaaaaa aaaaatatat atatatatat atatataagg cagatggtgc ctaagagcat |
| 185941 | aggatttgga accacatcgc tctgggttca aatctaagct tcaccaccag tcccagctga |
| 186001 | agctattcaa gaccagaagc tcatctgtgt gaacttccat tttcccaaca ttgtagggtt |
| 186061 | gatgtatcct aaggaataaa gaacaaaatg cacgcagaat ctttcacacc atgtctggca |
| 186121 | cacagtgaat gcttagtaaa tggcaactaa gaaattgctc ggtactgtta ttctattagt |
| 186181 | ttggttctat cagacccagt cactaaataa accagtttga cgtgaagtac ttggctatca |
| 186241 | gcaaatacta ccaggccctg tcaagtccca ggaatcagag gggctgtttg gccaatgcag |
| 186301 | actgaccctt tcatgctgac gtcagggagc cccagcagag acaaccaaag atctagagac |
| 186361 | atgatcaaaa tttccctgtc aggacatggt ccagaaccat ggatctcagt ttactttccg |
| 186421 | cagaaagaag aagaaaaggt gaagcccgca cacttctctc ttccacaaac tcaggcccca |
| 186481 | ttcccttgta cgtctttctc ttataaacaa ttgcacttta ttttcaattg tgttgtgata |
| 186541 | aatttgagct atagcttaca gtcaacaatt tctatcacaa atctttctgc cttccaattc |
| 186601 | tgtgtatgta caaactgagg ctaggaccaa aggaacRaag actttatctt acatgaatca |
| 186661 | tttgattcca ttactgaaat ctctcaggtg aagttaaaat atatatacac tgggtctata |
| 186721 | ttagaatata tgagacttga atttgatctg ctaattagct gggtgacttt aagaaagtat |
| 186781 | gtaacttatc tgagcatccc tttccccatc tataaaagga ggcagaggcc aggtgcagtg |
| 186841 | gctcatgcct gtaattccag aaggcggagg caggcagatc acttgagctc aggagtttga |
| 186901 | gaccagcctg gacaacatgg cagaacccca tctctaccaa aaatacaaaa aattagccag |
| 186961 | gcatagtggc atgggcctgt ggtcccagct actcaggagg ctgaggtggR agaatcactt |
| 187021 | gagcccggga ggcggaggtt acaataagcc aagatcatgc cactgcactc cagcctgggt |
| 187081 | tacagagtga gatttattta tcactaaata aataaataaa taaatggaga caggaaaatc |
| 187141 | tgcctcttaa gactacagca tgggttaaat agcatgtaaa acctttggca tgttggattt |
| 187201 | ctagagcatg tactatgtct cagacactac aataaacatt gtatgtgcat taattcattg |
| 187261 | aatcctaata acaactctat gaggtgctgt ttattattcc tattttccta gaaagtagac |
| 187321 | ctcttggggc cagggatagt ttttttcatc ttcaactccc aactcaWata ccctgtgagt |
| 187381 | ggtaaatgca agctacaact atcattgtcg atagttgagt ctggaatcac cgcagacgat |
| 187441 | tttataaaga tggaccccaa gaggtaggga ggtttagaga aaaggaggaa ggagagtgtg |
| 187501 | caggtcttcc caatggggta aggYRaggca tgaggaagtg acaagaaatg ccaagccagt |
| 187561 | aagaagacag gacgaagcat cgcacctgga tagaagacta agtaaaacta ccttcctgtg |
| 187621 | gctaaggggt ctactaggat acactcattg acttgctttg gccaacagaa tacagtggaa |
| 187681 | gttagattac accagttcca agctcaggcc tcaagaggcc ttgcaagctt cccttctgct |
| 187741 | ctccgggaac cctgcctagt tgcttagcat atgaatacgc ttgggctagc ctgctggaga |
| 187801 | atgagcagac acagaacaca aacaagctat cctagatgaa gctattcatc taggacaagc |
| 187861 | tgactgcaga cacataggtt aaccagccca aatcagaaga accagctagc tgagcccaaa |
| 187921 | tcgaattgcc gacccacgga atagtaagca aaataaatgg ttgctatttg aagccattaa |
| 187981 | gctttatggt gttttggtat gctgcaaaac taacagaggc aggagtcaat gtctcattca |
| 188041 | tctttatatc ttcaacagcc aataaaagtc ctggcatgta atagttacac agtaaacgct |
| 188101 | tcatgaataa agtattgaac tgatatatca atacagctgt gaataaataa atgtcatatg |
| 188161 | ctggaaaact gaaatcatgt aagatcgcat aattctattg agatacttca gcttaaagaa |
| 188221 | tatttttaaa tggggttggc tgctacctaa ttttaaaatc cattttcttt tttcttccta |
| 188281 | gccaacatcc ttttccacaa agtttagtca aatagaaaag aaagtttcac atgaacatac |
| 188341 | aacgtaggat gYggtgatcR aaaactagac tggttgctta gagatccgag ttctagactc |
| 188401 | aactctccca ataactggga gaccttgcta attctcttaa ttaccatttc tacgtctata |
| 188461 | aaatagatat taaaaaatta cccttccctg tcaacctcac agggttgttg tagcaaataa |
| 188521 | gacagcatgg gaaggctctg agcacttttt accgtcaatg attctacttc cagccaggct |
| 188581 | cagtggctct tgcctgtaat cccagcactt tgggagtctg aggtgggtgg atcacctgag |
| 188641 | gtcgggagtt caagaccagc ctggctaaaa cggtgaaacc ctgtctccac caaaaataca |
| 188701 | aaagtagttg ggtgtggtgg caggcgcctg taatcccagc tactcaggag gctgaggcag |
| 188761 | gagaattgat tgaacccggg aggtggaggt tgcagtgagc caggatcgca ccacggcact |
| 188821 | ctagccaggg aagacagagc tagccttcat ctcaaaaaaa aaaaaaaaaa aaaaaaaaaa |
| 188881 | aagattctac ttcctctata agcagaactt tactttttga tggtttctag tgtttttata |
| 188941 | atatatcctc ttaagagccc catttatcct tttgctgtct tccttttaat attctgtatc |
| 189001 | ccagtagcca tattttcaac atacatacat aYatacatgt atgaaaccaa gataggaaat |
| 189061 | ggttaaaatt atccttgata gtggagaaaa ctattgctcM atccaaagag agctggtatt |
| 189121 | tctggcactg aggattacat gtgaccaagg tcagacatga tacctgcaag atgtatcctc |
| 189181 | tagctccaga gttcccatgg agaactcggg gtccctagtt ggctgcagca ggggctgcca |
| 189241 | gctgctaagg atgcagccag atccctttcc ctttccagag acttctgccc agtatgggaa |
| 189301 | gtcagaagct ccacactcca agtatggctc ttccacaacc tcagttcctg ccttgggcca |
| 189361 | agtcttgaga tcagtgtagc tgagctccga gcccaggtct ggcactgcta aatggaaatc |
| 189421 | cttcatcttg gcaactcctt ccaYaatggY agaaccaggt tggaagttca caaggtgctc |
| 189481 | caggcatatc agggcttact gccctctcct aaagctcccg aaacctttgg aagtcacagt |
| 189541 | tctggagtaa acatcaggtg tcctcatacc caagcatctc aggagtgtgc aggaggcagc |
| 189601 | agaaggtagc ctgtgtcctg aggcagtgca ttctccccat cccatgaatg atggggctgg |
| 189661 | acagaagaca tcagggacag caactcaaac cactgggcag gtggggctga atggatgggc |
| 189721 | aacactggcc gtgctcaggg ctctgggtct tccttgacag tgcaattgta atacctccat |
| 189781 | ccttccatca acccatcctc cctatggtgg acataggttg tctgcctgcc cagataccat |
| 189841 | tctacctcct tctagtaaca atacctccat tttctacctc tccctcacta tcagttgtgt |
| 189901 | aaaatcaacc ccactctacc ctccaacttc agagacaggc ttgtgactag gcctggccaa |
| 189961 | taagaaaacc ggccatgttc tagccagcca tggtgactca cacctataat cctagcactt |
| 190021 | tgggaggctg aggaaggagg attatttgag gccaggagtt caagaccagc ctgggtaaca |
| 190081 | tagtgagacc ttgtctctac aaaaattggc caggcatggt gatgcacacc tgtagtccca |
| 190141 | gctactctag aggctgaggt aagaggatcg cttgagccca gtgagccacg attgcaccac |
| 190201 | agcactccaa cctgggcaac agagcaagat catatatgta tatatatata tatgagaaag |
| 190261 | agacccacat cccttagtga tccgttcaag agtgatcagt tcaagggtga aaatgtggtc |
| 190321 | tgaggcaggc aaattagagt caatcctgga acttctgggg gaaagaggca ctttctttct |
| 190381 | cttagtatgg aaaggcagat aacaatatat acctgaagca gctgggagcc Mcccatcaag |
| 190441 | caggcctgtt tacaaaggat gccaatgtaa agaaaagtgg agtttagaga tgaaggagag |
| 190501 | atggagggag tcaagaggac cttgctgagc ccctagaccg agtcacgagt gaagaagata |
| 190561 | cactcacgag ctgttagtta catgagccca cacattcctc tgtgtgctta agcactatga |
| 190621 | gttgagtttc ttgccatttg caaccttaaa aagccctgcc tgatgcacat cccaccattt |
| 190681 | gatgttacca acagactcta ataaggctga ggaagaaccc aggggcccca tccaaatacc |
| 190741 | tctaggctgc ttgaactcaa atgcttttga aataaggatg gttcactgtg gctgtcagat |
| 190801 | gccacacatc aaWcctgaaa ggaaattcta gccagggaat gctacagatg gcagctcacc |
| 190861 | acggccccca ccactcctta ttgtctttgt caagcccatt tcctcattta ctttacctgc |
| 190921 | ctggtccctt taaatacata agtttaccat cccttattag atggataaat agataaatgg |
| 190981 | atRgatggat ggatggatgg atggatggat ggatggatgg acggacacat ggatggatgc |
| 191041 | atggatgcat ggatggatgg atggacacat ggatgcatgg atgcttggat ggatggatgg |
| 191101 | atgcatggat ggatggatgg ttgcatggat ggatggatgc acagatggat gtatggatgc |
| 191161 | atggatgcat gcatgcatgg atggatggat gtgtgcatga atgaatgcat gcatgcacag |
| 191221 | atggatgcat ggatgcacag atggatgcat ggatggatgc atggatgaac ggatgcatgg |
| 191281 | atggatgcat gaatggacgg atgcatggag gaatggatgg atggatagat gcatgggtgg |
| 191341 | atgcatggat gggtgcatgg gtggatgcat gagtggatgc atgggtggat gcagggatgg |
| 191401 | atgcatagat ggataggcag acaagcaagc agttatgtag ttgcaagttg aacctacaca |
| 191461 | caaataggca tgatacaact aaagaattaa aaYggcagcc cactgcagtc agggggccat |
| 191521 | ctgacaatat aggaaaggag ataactatag gcttacttct gtatggtttt gggtgtctgg |
| 191581 | aataacgRga gatgactctc tgggtagcac tcaccctgta gttgtgctcc tccctctcaa |
| 191641 | gccacagtgg ttaacaccac cttaccaata gtgcagtaca aagagaagcc agtcctggca |
| 191701 | ttcacaggta gctgtgaaaa gctggcccta aaaggcagtc tcgctcaatc cccagcatct |
| 191761 | actYggatct gggcaggcag ccacagaaac agaagaagct acagaggatg Saggcactgg |
| 191821 | gttttggtgt ctggacatgg aagaattgca ggccaatatc cacaaatgta tccgtgaggc |
| 191881 | tctggtcttg aaacaaggat gtgaggaccc caggatggct ggtgagggac actaagtggc |
| 191941 | tcaaagaact gagaaaaaaa taaaaacata gcagcaatag agaccagagt ccgagccagg |
| 192001 | acaaggcctg tccctcagcc acctggctga aggcatcatc atgacctcaa ggaggtaact |
| 192061 | ggtgagtcat acagaaccac actgtgcact cttcaccatg gtttatatgc tcacagttca |
| 192121 | ggcaaYtgct tagaaaactg catatcgttc atgcccctat gaatcacttt cctccttttg |
| 192181 | ttgtctttgt gaagtcatca caaagttctg agtggaagaa ttcttccagg gactgccaag |
| 192241 | gagtgacact gtagccaggt gggttcagct ctcactttgc tcaggatatg ggtgtccagt |
| 192301 | ggcgacagat caggaaaata gccccatact aaatggtgcc tggacaaggc ccataMacac |
| 192361 | ttggatatgc tgtcctgaac acaaggcaca agacacaaca cgggaggacc tttcatctca |
| 192421 | tgtctcttac agccagggac tgaaggtgta ggacagtagg agaaaataga gtttcacata |
| 192481 | aaacacccca aggacatcaa agaaggctag ctgccaagag gtaggaagaa ctagattctt |
| 192541 | agggacttat ccttggggat tgataccacc cctgccaggc atccaccttt ggggtgactc |
| 192601 | agagccaaga aaacaaaggc atgggataag ccatggaaaa caaattcctg ctgagtagca |
| 192661 | aagagagtgg caccaacaag tggcaccatg tgagtgacaa gagcaagcaa atgctcacac |
| 192721 | agggacagct gggtgtgcag ggccatcaca gctcccccta cccccaccac ctgaacacta |
| 192781 | cgagacaaat gggactcctg gtttctgccc tcaacatgct tatatttgag agaggccaca |
| 192841 | ggaccagcga gtgcagagtc atggcgaact gtacacaacc tggagtcagg agacccatgt |
| 192901 | tctcaccctg ttctgttgcc tcgggggccc tgaagcccat cgctaagtgt ttKgggctcc |
| 192961 | agtttgctca tctgtcaaga gagaaccctt cacacagctc ctaagtgctg tgagtccctg |
| 193021 | tctctcagac tggctaatct ccaagggcct gccaggtccc acatgcatgc tcttgagagt |
| 193081 | cacatagata tcaaagctca aaattagctc ctgggagtgg gagcaaagct tctgagcccc |
| 193141 | cagcccctcc tgtgagcaca tacctcaagg cacatgcccc caacagccca gaactgacac |
| 193201 | atttttgata gttcctgagt acatggattt ccagccctgg aaatgtttgt tttaacttac |
| 193261 | caggagaaaa gaatttggat tcttttttaa atatacacaa agggaataca tttgtatatt |
| 193321 | ttcagataca aagtggatgg gaagcaaggc taaagactgt tcttttccct tagctcatgt |
| 193381 | gagtgctatt cctcacggtg aattctccaa agaccatgaa tggcagagat ctttgatctc |
| 193441 | ccttctagcc tgtctcgatg tgatagcctg gacatgggca gctatcacgc cacccttcag |
| 193501 | aacctcagca cttcaatgac cacatcctgc catcatcacc cccatggagg tccagggccc |
| 193561 | ctgccccact gcctacctct ccacctgtgc ccacctctcc acctacccgc cccttgtcac |
| 193621 | attgacagat aaaatacagg actcccagtt aaatgcaagt gtcagatgga gaacaaataa |
| 193681 | tattttagta taacatgtgc cttcctaggc agcacaactg aagcaggccc cttccaagga |
| 193741 | tagaaaagtc ctcctctctt cctaagtttg tgtccataga atctcatcca gtggcctttc |
| 193801 | tgtcccctgg cttcaaatga aacctgccac tgcctctctc tcagggtgaa gtgtgaagac |
| 193861 | cgtggcttcc aaggccaata ggcatggagg tagtcatccc attcagacgc caatggttct |
| 193921 | tagtcaaaaa agctgcttat ggctggagtc aaaaacatgt ctaagcgtga ctatcagccc |
| 193981 | cagtgcagaa gagtctgggg agagggagaa acaagaaaca gatattacca caaaacactc |
| 194041 | caagggaaat ctcactacaa aaaaagaggt tgttgcctgg tgatggctgg caatgggcca |
| 194101 | tgaaaaccta gttctgattt ttagatacaa gaSacaagcc caacgtgtca cccagcctgg |
| 194161 | atcccataga ggtcacatgg acaaaacagt acttgaagat ctttgacctg cctaggtggg |
| 194221 | caactggagt ggaagctcca caagggtatg aatgctgtct tttgttttcc tcatctctat |
| 194281 | ggactagtcc atctagtcca ggactagcct cagagtgagc atccgatgat cgtaggaaga |
| 194341 | ctgaatgaat gcgtgaacac atgaatgaat gcctacattc tccaatatgt cccagaaagt |
| 194401 | gtcaagagct ttgtaacgca aaaggctctg aacaactcaa ataagaaaca caaacatgtg |
| 194461 | cagtgaaagg gctaggcctg gatggcctgt gaggtcattg gcaatgtggt gagcccttga |
| 194521 | gcctgtcata cacaggcaaa taacttcact gaaagaattt taaacgctac ctccaacacc |
| 194581 | ctagctcagc atctatggtt gaagccacca tgcccagtca tggtgataac cccagaaggg |
| 194641 | aagggtgcag agacgccaag aaacaagaac ttggagagga caatgaggac caaatgactg |
| 194701 | gggaaataag ggatgagtgg gtaggaatcc aaagaacaga ataggaaaaa taactcaaga |
| 194761 | tccagcactg ctttccagga cacacacaca cacacacaca cacacacaca cacacacaca |
| 194821 | cacgcagaca aagattcccc ccttccagat ccacactgcg tgcctcctgc cagccccatc |
| 194881 | ccatcatcct gtgacagtgc ttctttgtgg aatagaacaa taggtacatt atgccaacct |
| 194941 | ataattatcc taggagaagc gatggcgaag tcaaggcatt atgtgaacac tggaatactg |
| 195001 | gataccaccc caccaggagc aaacaacaac aaaaacaaca acaatccaca aaatctctaa |
| 195061 | cgcagataaa gactgaaagc tgcctaagcc agtgcccttt cacattcata tgagggtggg |
| 195121 | attttttcct tttcacatga ttatttaagc acaaggcctt ctttcatgga attgatagag |
| 195181 | tattggggga aggggctgag aggttgggtt tgtttgtgtt tttgtttttt ttcccaataa |
| 195241 | ggaggcagaa agtagaggag gtcgtgagca cgagaaagtg ccactgctag ggtatgacac |
| 195301 | atagagccct caaaggtagt tgaatctgat tcWactgagt gaaatcattt cattaataga |
| 195361 | gattccttct cactactcct gtgaaactac tttccaggga cgttggtata aaataggctg |
| 195421 | agtcctggga ggtgtcatgg aagacagaga caagaaatca gaatatctga attcttatat |
| 195481 | cacagccact aactagctgt ttccttgatt tattcaagag gtatgtgttc cctatctgcc |
| 195541 | acatgctagg catcaagcta gcaacagaag gaaaaaacaa agcagtagtc actttcctct |
| 195601 | cggagcttca gcttcctact gtgtaaaacg agggagtccc acagccctgg acacacNcac |
| 195661 | acacacaMac acacaSacac acacacactt ctaaatacaa ggtgcagccc cagcctccca |
| 195721 | caggacctgc ctatacaggc catgactgaa gaagggggaa acctgcagct tctaaaactg |
| 195781 | cacagaaggc tgggcacagt ggctYacacc tgtaatccca gcactttggg agggcgaggt |
| 195841 | gggcagatca cctgaggtcg ggagtttgag accagcctga ccaacatgga gaaaccctgt |
| 195901 | ctctactaaa aatacaaaat tagccgggca tggtggcagg taactgtaat cccagctact |
| 195961 | caggaggctg aggtaggaga atcacttgaa cccaggaRgc agaggttgcg gtgatctgag |
| 196021 | atcacgccat tgcactccag cctgggcaac aagagtgaaa ctctaYctcc aaaaaaaaaa |
| 196081 | aaactgcatg gaagcccaaa cccccagggc aaatggaaca gtcacagcat gccatgcacg |
| 196141 | cacagggtca gagcaagagt atggccaggc gcggtggctc acacctgtaa ttccagcact |
| 196201 | ttgggaggcc gaggtgggca gatcacttca ggtcaggagt tcaagaccag cctggccaac |
| 196261 | aaggtgaaat cccgtctcta ctaaaaatac aaaaattagt tggacattgt aatgggcgcc |
| 196321 | tgtaatctca gctacttggg aggcctgggt attagaatcg cttgaacctg ggaggcggag |
| 196381 | gttgcagtga gttgagattg tgccactgca ctctNcNtgg gtgacagagc aagactctgt |
| 196441 | ctcaaaaata aataaataaa ataacaaagg tgtgaaaagt aggatgggtg tttcctagca |
| 196501 | catctctagt ttctgagggc tctctctaat ggggaccYtc ctcttgaatt gtggtcccat |
| 196561 | gggttccaYc tcttctggga tccagtcagg gcttcttcct tcattctcag gcaccttcag |
| 196621 | Mcttgaaact ccttggcctc ctttcccttc accttcctgg tgaaacataa cagagggtct |
| 196681 | tcgagacaat tcctttaaat catcaaaggg cctatctagc aatgtctaac tccattcctt |
| 196741 | aacgtttact gaaaagctcc tcaaatacat gatcctcccc tctacccgtt cccctcagct |
| 196801 | cttagYcatc cttcctcaga tgStactttt tcatagggac taatttcttc cttcttgcca |
| 196861 | aatccagtgg ccacccgtgg gtggtcctgc tgctcaacca acgtatgaca gtattagcca |
| 196921 | tgcgctcctt gaaacactca gagccttgat tctactcatt catttagttc ctccccaaaa |
| 196981 | gttccctgag cccctgctag gcagtgggga tttggcaata agacccagtc cttagtcctc |
| 197041 | tgctcatctc tccacactcc gctcttatgg ctccaactct cacatcagca aagggctttc |
| 197101 | aaagactcaa agcacctcca cactcattac ctcttgtaat aacaaaattc aactctgcaa |
| 197161 | actttgcaaa acactttata tcgtaatcct tttttcccca cccScactgg gcgaaagaac |
| 197221 | ctattcttgc aaccaacact atcctgaaaa tgtggaaaac ctctaatgga agtgcaacca |
| 197281 | agtgctcctg gaattcgaca tgctttttta tgtccttccc acccctagtt ctcccctccc |
| 197341 | tgcccactct cccttcgcac ttcgcttgcc taccttacag cagagttcat tttgtcggtc |
| 197401 | tcttctaagt ggtgggctcc tggagggctt ccaaYggact gatctctttg tagactccat |
| 197461 | attactcggt gcttaaatat ttgttgaaat gcagcaagat cagggagtaa ttcagcacca |
| 197521 | attgtttacc tgatcctgga aacctcatct ctgccccctc cattccccag gagagtctgc |
| 197581 | ccatttgaaa actgttcctg ggtttatttc tRgcatggcc ctggagaggg ctgccctacc |
| 197641 | ccgcagggtc agggaggtga gttttctctc accttgggta ctgcagagcc caccaccagt |
| 197701 | ccaccctgaa acctcaaagc aggcccacag ggtagatatc tagggacctt gaagcaaaca |
| 197761 | tttcacctat tcaggacaca aactaaataa gcttgtgtcg cttagtaatt ctgtaatgtc |
| 197821 | ttacttatgc ccttgggcaa actccttaaa ataYttgttt ctgttttccc atcaataaaa |
| 197881 | tgagaattga agcacctatt tcttaggatt attgtgaaga tttgatgaat taacactagg |
| 197941 | aaatgtgcct agcactgttt aatatgtggc agaggctcaa taaatacatt attattatta |
| 198001 | ttactattat tgacaggtta aaggcctgtg atgatactga ctccagcata actctgatac |
| 198061 | tgcccctttg actcggctag ttttgacaga gggccctttg acaacaccta aaaaacaaac |
| 198121 | cactaaactt tcagttttta aaaaaatacc aggtaagctg ggtacagttg gctcacgcct |
| 198181 | gtaatcccag aacttgggga ggccaaggct agaggatgat aggaggatct cttgaggcca |
| 198241 | gtttttaaca accaactctc cagYgaacta atccattcct gccagaacaa atccagtctt |
| 198301 | acaagagtat gaatattaga gctacaaggt accgccaaga gacagggttt tgctttgttg |
| 198361 | accaagacca gcctggtcaa cataggaaga ccccatctct acaacaaatt taaaaattag |
| 198421 | ccaggcttgg tggcacatac atagtcctag ctactcagga ggctgaggca ggaagatcac |
| 198481 | ttgagccctg gagttggagg ctgcagtgag ttatgatcac gccactgcat tccagcctgg |
| 198541 | gcgacagagc aagaacctgt ctctaaaaca acaacaacaa caacaacaag taatgtgaga |
| 198601 | aacaaatgcc ctccctccct ccctgagggt cacccccagc ctgacctgat ggatgggaaa |
| 198661 | agataagctc agagatgggg taaggataag gtgggagtta ggaatggggt agaggtcagg |
| 198721 | gatggactga gaactaggaa agggaaggat caaagatgga ctggaggtct ggaagcagga |
| 198781 | agagagaaat ggagtaaggg tcagggctgg agaccagcag aaccgaggtg aaatKtagga |
| 198841 | ccatctgatg gatggtcaca ttaataactt taatttttca ccaccagctc ccactacagc |
| 198901 | atccaaacat cctgctctga aggagtggga gcagggacag ggaagaagaa gaaaccttaa |
| 198961 | ctgagtgaac attttaaaac aagcaggaca tttcgaactc aaatagtaag agtcactttg |
| 199021 | ctgtccaaat cccaccagtc cttggtgtaa tactacaagt gggacttaca agcaggaccc |
| 199081 | gcaagaaggt tctagtaact acctcagctt gcaggacgta aaatgcctgg cgaactttgc |
| 199141 | attccaagga caggagtctt ccaaattaaa attttaatag tgtgtgaaaa tagttcgcca |
| 199201 | tttactctta ggatgaggta gtgtttagaa agtcagcctg gaaatccagg ccaacatttt |
| 199261 | cagggatgcc tcctRttctg ggaggagaaa aaccagcttg ttgcttccct ccagtagcac |
| 199321 | aaagcagcag ggaacYggaa agagagaggg gtctgaacag cttcttctac caggaaacca |
| 199381 | gccaaaggtc tccgggcatg taaggacatc tgttttcaga agctatggtt tggattttgg |
| 199441 | cattcttagg agtctaggag caatcctact aatttgggtt ctagctccaa ccatactacc |
| 199501 | tacctgctgt gcgaacccag gcaagtgatt ttacctctct gagcctctgt ttgctcatct |
| 199561 | ggccctctag acaagattaa cttggcagtg ccttgtagct ctaatattca tactctcata |
| 199621 | agactggatt tgttctcaca ggaatgaatt agttccctgg agagttggtt gttaaaacac |
| 199681 | cagggccccc ttgggtttcc ccatcttcac acgtgtctac tttccctttg accttccctg |
| 199741 | ccatgttgtg atgcagaagg aaagccctcg ccagaggaca gggccatgcc cttgaacttc |
| 199801 | tcagcctgca gaaccataag ctaaataaat ctcttttctt tataaatgat ccagttcctg |
| 199861 | gtattccatt acagcaacac agacagacac tcccacatca tcRtcacaca gggcttgggc |
| 199921 | tgcctggccc tggccactgc ctgccaacac caggcctggt ccctccatca tctaggacct |
| 199981 | tggggaacat ggcaccggtg aagggcatgg gggttaYcag aaagagtgag aagtcggcag |
| 200041 | gtcagcacgg ccacacaagc caccctctcc caacttatgt gcaggcatga tggctcccca |
| 200101 | tccaccagca ctcctgaggc cactacacca gggaggaaag Wagtgcatgg ggaggcctgg |
| 200161 | tcacacctgc ccttcctttc agaaccctgc atgtggctga tggcaggtcc tcggcgcctg |
| 200221 | gtggaagaga aagggaaggt ggcactgggc tccacaggca gtgattctca gccctgcttg |
| 200281 | tcactgggag ctggttcctg ggtcccaccc caatctctta tgattccata gatcaggagg |
| 200341 | agacccaaga acccaacttt tcacttagct ctccagggaa taccaatgca aacggccact |
| 200401 | gaccacccct tgagcgccct gtggatgaga gggtggaact agaggttcta gaagcaagtg |
| 200461 | cttgagtccc ctcactagct gaatgactct gggcagttta catctccttt caaaccctca |
| 200521 | actgcccatc tgagaaatgg ggatataaaa gYgtctgctt cttggggtta tttagagaaa |
| 200581 | tacatgagaa aaaggatgga aagcacttag cactgtgcct gtacatgttg agaatctata |
| 200641 | agagaagatc ccctcttact attcaggggt gacttgtaca aatgccttct tccatagcaa |
| 200701 | tcccttacta aactggagca tttcataggg aagatgcgaa taagggcaat gaagcctcag |
| 200761 | atacagaggg cagcggagtg ctgagacacc tccgtgccct cccacaacat cccagtgctg |
| 200821 | tggtccagca acatacccgc cccatccctc caactcagaa catgctcRtt gatgtgcctg |
| 200881 | aacccaggct aaatgggagc acacctatag tgggggttcc cctgtcagag atatgcactg |
| 200941 | caagggcctg cggtccagga cacagaggaa ggaagagacg ggMggagaaY gtgaatcaca |
| 201001 | ttcatgccag ggtgtgttct aatacgtcag caaatgtgta ccgaScttcc gggctgcaga |
| 201061 | ggaaggtggg cgtgtgtggg gttagtgact tgaagggaaa gaggggcatg aaaaagaagg |
| 201121 | gggtgaagag ttagaactgg tgcagcagct gggtctcaca cacaaatggc atgactctac |
| 201181 | ttcacaggaa ctactcagaa agggaaaaga ataactctaa gaaacaaaga tctgcctcca |
| 201241 | aatccaagaa tggcagtaga aaatagggtc ccccacccag caagcaccca catcacgttt |
| 201301 | catcagaaga actctctatt ctcagccagg cctggcacac tgccttgacc ttcccgatcc |
| 201361 | tggagctggg ccctgggctc agaagacccc agactgtcat tgctggaaac agaatcattg |
| 201421 | cagatatagt tagtcaagat gaagtcacac tggaatagag tgagccctaa tcttacagga |
| 201481 | ctagtgagtt agtccatttg tattactata aaggaatgcc taagacttgg taatttgtaa |
| 201541 | agaaaagagg tatatttcac agttctgcag gctgtagagc aaccacggtg ccggcatctg |
| 201601 | cttctggtga ggcttcagga agctacaatc atggcggaag gtgaaaagga gagcaggcgt |
| 201661 | gtcacatggt gagagcaaga ccaaggcagc aagggtggga agtcccagac tttcaaacaa |
| 201721 | ccagatcttg tgtgaactaa ctgggtgaga cttcactcat caccaagtgg acggtgctaa |
| 201781 | gccattcgtg agagatccac cctcatggtc taataatctc ccaccaggcc tcacctccaa |
| 201841 | cattgggaat cacatttcag catgagcttt gcaggggaca aacattcaaa tcatatcaat |
| 201901 | tagtgtcctt ataaaaaagg gaagtttgcc ccatgaagga agagattggg gtgatgtgcc |
| 201961 | tacaaagcaa ggaatgccaa agataagact gccagccaag ccccagaagc cagcagagag |
| 202021 | gcctggaaca gattcttcct cacagccctc agaaggaacc aaccttgcca acaccttgat |
| 202081 | cttggacttt tagcctccag actatgataa aataaatttc tgtgaagtta cccagtctgt |
| 202141 | ggcacttaca gccaccctaa caaactaata cacccagaac cttactatcc ttcaccacac |
| 202201 | cgatcaccaa tgaggcaagt cagaaatttc caaaaagtct ccacatttat ttttctaccc |
| 202261 | aataaaccag acatgataga ggaattcaaa tataaacaag acataggccc tgccttccag |
| 202321 | agctcctagg cacacaaatg ttagtcactt tacactttgc aaaacacttt cacatatatt |
| 202381 | gtctcttgta gatcccccca tctcacattg agatgggaag ggcaaSctgt aggttactcc |
| 202441 | cattctgcag atgagaacac tgagttttgg agagttggtg gcttgctcca gggcacccag |
| 202501 | ccctggagcg gcagagaggg agctgcagta aagtcttgag tccaagacaa cattggtgtg |
| 202561 | gctgttttct ttcagcctgt cttgctgcct ctcctgccgt cttggaacct cttgcctcct |
| 202621 | tcaaacagaa gcccctgcac tgaggtctct tgtatctaag atctgcccca agtgagctcc |
| 202681 | aggaggcaag gagggaggcc agccatgtta aagaccaagt taaagaccaa agtggctgca |
| 202741 | gcaccatgag ataccagtgt agtcccccac cttctctcat agtcccttac cctgaggctg |
| 202801 | ctcccaccag ccccgcttac ctttttccca tccccacccc Mggcccccag cctctcccat |
| 202861 | cccacaggca gggcaggttc ctggtaacag cattgcccag tgttgtcaaa atagcgagga |
| 202921 | gctggaaaga attccagaat gaaatctatg tgcctctgca gcctcccaac tgactgccac |
| 202981 | tgtggcagct aaacccgcta atgtgctaag cgttttctgg cagggccagg aggcagggtt |
| 203041 | ggtattttgg gagctgggag gggcttggag gcaacatcct cggcaagtct gggaaggagg |
| 203101 | tggaggcagg gaggcaaagg gaaccgaccc agaacacttc ccatgacgtc accgagagcc |
| 203161 | acctgagctg cacctcagga ttcctgaaac actgctggcg gagcggagag tacggagagt |
| 203221 | acgctgggcc cggtgggcag ggctcagcag ggcatggagg ctttgtagct aggcctgggg |
| 203281 | ggagagggcg ctctagtatg ccacttgtca Ygtacacttt ttctgctgac acagagcagt |
| 203341 | gtgcaatgcg tccgggaggc tgggcagccc agggctggcc caagctctgt ttgcctgagt |
| 203401 | tggcagtggc agggcccatg gtgaccaaac aaaagacggg aagccaaacg cccagaggct |
| 203461 | ggcgggtgag gctccctgag agggctctga ggatacaggc actggcgaag aagggccagg |
| 203521 | ggctcatagg gataggaatg ggKttggggg caatgggaac ttgcaggcag ggtcccctct |
| 203581 | ggtttcccag acacaccgca tcgcctctgg gcatcagcct ctccacactg tccttgcttt |
| 203641 | tcgggggctc cttcgaaaat gcttcaaacc atgacaccag gaaggggagc aaaccaccaa |
| 203701 | atctgagatc caaacaaggt cccagcattc ctttgtccct gatgggctYg gctccaaatt |
| 203761 | ctgggagaaa ccagagggtt ttgagagagt caggagaggg gttcagggga caaaaaatag |
| 203821 | ggctggtgaa gaattccttc cgggagccgt ctggtagcag ggagtaccgt gggtggcaag |
| 203881 | gccgtgctgc tcaggtgagg ggacatggtg tcattctggg aacagatgag ccagtaaaac |
| 203941 | cctgacagga ttcaatctgg aggaaaaaaa tcccagctga taggaactgc tgagcgcttg |
| 204001 | cagcttttaa agcccctcta gcaaaaggat ctgcaaaagY tttgcaaact ctgaccacgc |
| 204061 | agccagtcag ccacttccca gcccacagca acgagagagt ttttgctaca tcagaacaaa |
| 204121 | cccactcaca aatatctcat cctggcctcc cccccggcac ccattccttt tgggtctgtg |
| 204181 | tgcctgcctt cctcagaaat gacattttgt cagagcagtg agagatagtg ttgcaggggc |
| 204241 | caaaggcagc cagggggctg gactgcaaMt aggctcccct gctcactagc ggctgacccc |
| 204301 | aggcagggYa cttaaccttt atgaatctca ttttttaatc tgcaaaaatg caaataatga |
| 204361 | agctgctctt acagagttgg tgagaggact aaatgagatg atggatgctg gggctcgagc |
| 204421 | acagaRatga ggggctccat gacggctggt ccccctcctt cccaccttcc tctccagggc |
| 204481 | cctccggcac cataatcagt gtgggaagga tgcagaacaa tgggagttta attcagaggt |
| 204541 | tggcgaggca aaggcatact ttaatagaat tctctctcgc ccagtacatg gtttcttgtc |
| 204601 | caccccaccc acctttcccc atctctaccg gctgatagtc tctcagctag tagacctttt |
| 204661 | cttgtttagc agggccactt tttttaaaaa ctccagacgg gtaccctcca tgttgcaggc |
| 204721 | gacgtggccc tggatcactc aactgactgt catttgaatg gtgcccccag agtgaggcaa |
| 204781 | tggtgcagcc ctcctaaggc ccttcctgag tgtccctcct tcatgaagat gattctgagg |
| 204841 | cttcccaggc cttcaccctt ctttgaaagc ccatagcagt tcatatgcac ttctcttcta |
| 204901 | tgctcaccaa actctgcctt catcatactt gggggatgtg tgtgagagcg agagagagag |
| 204961 | tccagcaggg attagatctg acttttatat tctctacagt gcctggcaca atgctaacac |
| 205021 | atttgatgag gccagaatta ccctgatacc aaaaccagtt aaagacatta ccaaaaagaa |
| 205081 | ctttttcaat attttaattc ttacctcata ccctagataa aaattaactt gagcSgggtg |
| 205141 | tggttgctga cacctgtaat cccagcactt tgggaggctg aagcagacgg atctcttgag |
| 205201 | accaggagtt tgagaccagc ctggccaaca aggtgaaacc ccgtctctac taaaaatata |
| 205261 | aaaattagYt ggtcatgatg gcaggtgcct gtaagcccag ctacttggga ggctgaggag |
| 205321 | aaYtgcttaa ccgggaaggc agatgttgca gtgagccgag atcRcgccac tgcactccag |
| 205381 | cctaggcgac agagcgagac tccgtctcca aaaaaaaaaa aaaattaact cgacatggat |
| 205441 | cgtagagcta aatggaagac taaaactttc aaacttcaga aagaaaactt ggaagaaact |
| 205501 | ttttctaacc ttgggttaag caaaaatttt cttaaatagg acataaaatg catgagtcgt |
| 205561 | aaaggaaaaa aattgataca ttggacttca ccaacatttt aaatgtctac tctttgaaag |
| 205621 | gcactaatat gaaaatgaaa acacaagcta cagactgaga gaaaatattt gcaaaacata |
| 205681 | tatctgataa aggatgttta gtcagaatat ataaagaact cctaaaactc aataagaaga |
| 205741 | caaatgactc catttctttt aaggtcaaag aactgaacag acatttcaca aaagaaaaga |
| 205801 | tacagatggc caaaaagcac atcaaaaaaa aaaggttcaa cattattagt tagcagggaa |
| 205861 | atgcaattaa aatcacaatg agataccgtt aaacatctat tataatggct aaaatttaaa |
| 205921 | aggctgacaa tgccaagctc tggcaaggat atagaataac tacgactctt acacactgcg |
| 205981 | tgtgggaatg ctaactggta cagccacttt ggaaagcagt ttggcaattt cttttaaagt |
| 206041 | taaatataca attatatttt catttctccc agagatttgc tcaaaagaaa taaaaacata |
| 206101 | tgtctacaca aagacctata aataggaata ttcatagcag ctttctgcat aatggccaaa |
| 206161 | acttgggaac acccaagtgc tcatcagttg gtaaatgaat aagcaagttg ccatgcagcc |
| 206221 | acacagtgga acgcacccat acatgcagta acatggataa gtcttaaacg catggtatca |
| 206281 | gttcctattg ctactctaac aaattactaS aaactcagta gcttaaaaca acacagattt |
| 206341 | actgtcttac agttctggag gtcagaacgt ccgaaatcag tctcactggg ccagcatcaa |
| 206401 | ggtcagtttc cttatctttt tcagctccta gaagccacct gcattccttg acccatggcc |
| 206461 | ccttcctcca aagccagttg tgttgcatct tctatctaac tgcttaaggg aacaggactt |
| 206521 | tatcaaggca cagagccaca gcaagtatgg gaaacatata agtagttcca tatttccagg |
| 206581 | gtgtgtaatg taacatttcR aagataagga aactgagatt cagagagatc caatatctta |
| 206641 | cccaaggtcc aggacacagt gaaaatcatg ttcaacccta aggcctctga atccaaatcc |
| 206701 | agagtccacg tgagccctca gacttccctg ccttgaccgt ggatagtcat ctccaccttg |
| 206761 | tctcagagcc ctgcactccc accctWgtac actgcRccag cagctaagcc tgtggagcca |
| 206821 | ggcagaaccc tgggcctccc cacctcccca ccctgggacc aacagattcc cagctccagc |
| 206881 | aggataacat cataacctta ataagattga catttgcatc ctgttttata ttcatcagca |
| 206941 | ttcttacact tacgttatgt tatctggcct tctccatcgt atgagtctaa tggtgcagat |
| 207001 | attctccacc ccacattctc caaccctcag aaagctgagg tactttctta tacagcaagt |
| 207061 | gggtagaagc caagatcaga accttacccc caacccaccc cttgatacca gaagagccta |
| 207121 | ttgactcaga acaaaactca ggcaaacccc ccacctagct gagcttcccc tccttcatct |
| 207181 | gcaggatgga aaacagggta atacctgccc tgcctatcct gatacatcag gcataaatgg |
| 207241 | aaaaaaaaaa aaggaagtag tatgtaaaag cactttggaa atgttaaaat gctatgcaaa |
| 207301 | ttataaggaa ctgttctgat tatactctca agaaatgtca caaattttct ccaaaagtga |
| 207361 | ataattgtca ttacttttca acttcatccc cttgaaaagc cagtttctcc tcctgtcatc |
| 207421 | ttttatttct ggttaaagca gagatgttat actcaacatt ccaactggaa tatcaagatc |
| 207481 | aatctttaat tcctccttcc agcttcaccc tttctgaaat caagaacagg caattctcta |
| 207541 | ctccacccac catctcatcc tcagtttctc ttgccatctg cccccctctg aagctagaaa |
| 207601 | tcaccagcaa ccattttcat tcctcatccc tcYggtctct cctcagtcag agccaaccta |
| 207661 | catgatgcaa aagcaccact ccaatcacct ctctcagagg ctcacagccc cggggggaca |
| 207721 | gcRtccaggg gtcctccacg ggctgcccca gtgaagctct ccagcttgtc tcccatgact |
| 207781 | caccaaccca ggtaagccta aatggccctg tttcccaaaa ctcaactctc ttcccacttt |
| 207841 | tatgtcccca ctatatctgg aactcccttt cccactattt aattagctat gggggtagga |
| 207901 | agaatcaaac tagaccccca gctcttacat caacctaaat atattccaag tgaatcaaag |
| 207961 | aattgtggtt aatggctggg cacggtggct cacacctgta atcccagcac tttaagaggc |
| 208021 | caaggYgggt ggatcacctg aggtcagagg ttcgagacca gcctggctgg gtaacatggc |
| 208081 | aaaaccccgt ctcaactaaa aaataataat aataatacaa aaattagcca ggcatggtgg |
| 208141 | cgcaagccgg tagtcccagc tacttaggag gccaaggcag gagaattgct tgaacccacc |
| 208201 | cagcagatgg atgtttcagt gagccaagat tgcaccattg cactccagcc tgggcaacag |
| 208261 | agcaagacac cacctcacaa caaaaaccac acacacacaa agaattgagg ttaaaaaaaa |
| 208321 | tccagattat atagatgaat atctatctga tagctgaatg gggctggaat ttctaggcta |
| 208381 | catagcaatg gagtaaatca gaggagaata aaatgaataa tagttaacat ttacctattt |
| 208441 | tcacttcttt taactggata tccatatttt aaaagtaaac atacccttta cttcatgttg |
| 208501 | tacacaaaaa ataacaaaat ggatcacaga atgaaagaaa tgctaaaatt agaaaaattc |
| 208561 | cagaagaagg ccaagcacgg tggcctgtaa tcccagcact ttgggaggcc aaggtgggca |
| 208621 | gatcacYtga ggtcacgggt ttgaaaccag cctggccaac aYggtgaaac ccctattaaa |
| 208681 | aatacaaaaa ttagctgggc atggtggtac acacatgtaa tcccagctat tcaaatggct |
| 208741 | gaggcacaag aatcccttga acccaggagt cagaagttgc agtgagcagt gatcgagcca |
| 208801 | ctgcactcca gcttgggcaa cagagcgaga ctctgcctca aaaaaaaaaa caaacttcca |
| 208861 | gaagaaaaca tggggaaaat cttcataacc ttggtgtagg caaagattct taggacacag |
| 208921 | aaaataagaa tcacaaaagg gaaaaataac ctggacttca aaattaaaaa tttctgctct |
| 208981 | tcaaaagaca ttgttatgct gggtgtggtg gctcatacct gtaatcccag cacactggga |
| 209041 | gaccaagacg ggaggatcat ttgagaccag gaattccaga ccagcctggg aaacacagaa |
| 209101 | agaccctaWc tctacaaaga agtttattaa ccaggtgtgg tggctcatgc ctgccgtccc |
| 209161 | agctacatcg caggctgagg cgagaggact gcttgagccc acgagttcga ggttacagta |
| 209221 | agctttgatc atgccattgc actccagtgt gggcaacaaa gaccttgtct ctaaaaagat |
| 209281 | aaaaatttaa aaattaaaaa gacattgttt aagataggag atatggttgt acaacaatgg |
| 209341 | gagtataatt cacaccacta aatactcaac atgattccaa tggcatcttt tatgttacgt |
| 209401 | atattttact acaatttttt taaaagatgt tgttaagagg atgaatagac aagccacaga |
| 209461 | ttgggagaaa tgattcatag tacatatatg tacatagtat agtcatacag tatatagata |
| 209521 | catgtataca cagtacatat atagcacata catgtacata catacatata tgacaaagga |
| 209581 | tctatatcca gaatatataa agaactctag caatttagta ataaggcaaa caacacaaca |
| 209641 | aaagatttga acagaccctg cacaaaagaa gatagccaat aagcacgtga aaatatgctc |
| 209701 | agcatcatca gtcattaggg aaatgcccac taaaactaca gYaagctacY gttcacaccc |
| 209761 | attaaaatgg ctaaaattca aaagactggt aatgtcacct gctggtgaga atatatRgca |
| 209821 | accaaaagca cagccactgc tgacgggaat atgcaatggt gcaaccactt tagaaaatag |
| 209881 | tatggtagct gcttattcta aaaacatgca tttaccatat gattgagaag tttcactact |
| 209941 | aagtatttat ccaaaagaaa tgggggaaaa tggtatgtca aaaaagagac atacacagat |
| 210001 | gctcgtggca actttattca taatagccaa aaactggaaa caacccaaat gtccatcaac |
| 210061 | tggtgaatga ttacaatatt ttggaaaatt catatgatga aaaactcagc aaaaaaaaag |
| 210121 | atgttactgc attggtatca ggactgaaaa aaataaaaat aaaaattagc tgctaatgta |
| 210181 | cacaacaatg Yggataagac ttttttttta aaaggccgag tgaaataagc tagattcaaa |
| 210241 | agagtagata gtggtcaggc atggtggctc acatctataa tcccagcact ttggaaggcc |
| 210301 | tggggcgagt ggattgcttg agcccaggag ttcaagacca gcctgggcRa catgggaaaa |
| 210361 | ccccatctct actaaaaata caaaaattta gccaggtgtg gtgatatgta cctgtggtcc |
| 210421 | cagctactag ggagactgag gtgggaggat cacttgagcc caggaggcaa aggttgcagt |
| 210481 | gagctgagat tgcaccacta cactccaacc tgggcgacaa agtgagaact tgtcttatta |
| 210541 | aaaaacaaaa ggtagatagt gtatgattgt gtttacatga actcttagaa tcagcaaaat |
| 210601 | taatctacag ttacagagag ggggtgagta caaagaggca ctaaggaact tttggggata |
| 210661 | ataataatat tctattgtgg ttacatgggt atacacattt gttaaaattc atagaactat |
| 210721 | ataYtttgaa tgagtacatt ttattgaatg taaattatac catagtttta aaagaacttt |
| 210781 | ttaaaagtca agatgcaaac tggggaaaat atttgccaca tgtatgacaa attaatattc |
| 210841 | ttaatataca aaatgtcatg caaattgata taaaaagcat taagacctca acaggcaaat |
| 210901 | ggccaaaaga cacagaaaag ccacaaaaga ggaaataaga atatctaata aacaggttaa |
| 210961 | aattaaagca atgaaatact gtatttcaca aaccaaatga gcaaatttca cctatcaaat |
| 211021 | gagttttttt gtttttgttt tttgctttga tgataatttt tgttttgttt ttactttaat |
| 211081 | gataatacta gtattgtagg acaagatgag cactcttctg gtaaaaatat aaattgatac |
| 211141 | atttctagaa cacaatttgg aacactatta tataagcttt agcaggaaca ttaaagttaa |
| 211201 | gacccaaaac cttaacaatt ttcagattat ttgacccact tctagaaacc tgtgctaagg |
| 211261 | aataaacaaa gatgaatgat caaaattaga gatttacagg caagagtttt cagccaggca |
| 211321 | ctgtggttca cacctataat cccagcactt tgggaggtca agctgggagg atcacttgag |
| 211381 | gtcaggagtt cgagaccagc ctgggcaaca tgatgaaact ccatctctac aaaaaatttt |
| 211441 | aataattagc ccaggcacag tggctcactc ctgtaatccc agcactttgg gaggctgagg |
| 211501 | caggcagatg acttgagacc aggagttcaa gaccagMctg gacaacatga Kgaaacccca |
| 211561 | tctctaccaa aaatacaaaa attagccgag tgtggtggca catatgtttg taatcccagc |
| 211621 | tgcttgggag gctgaggcat gaaaatcgct tgaacctgRg acgcagaggt tgcagtgagc |
| 211681 | tgtgatcacg ccactgtact ccagcctggg caacagagtg aaactctatc tcaaaaaaaa |
| 211741 | aaaaaaaaaa ttagcgKgcg tggtggcatg cacactactt gggaggctga ggtgggaggc |
| 211801 | agagcctggg aggcagagtc Wgcagtgagc tgaggtcaca ctactgcact ccagcttggg |
| 211861 | caacagagta ggactgtgtc tcaaaacaag aaaaaacaag tctggccatg gcggctcagt |
| 211921 | cctataatcc cagcacttaa caaggtagag gcaggaggat agcttaagcc caggagtcca |
| 211981 | agacctgcct gggcaacata gcaagacccc attctccacc aacaaaaggg acaaaaaaga |
| 212041 | atttttatta ctgaccttgt ttctagcaag aaacaatcta ggataagcca ctaggttgag |
| 212101 | taaacgctgg catcaatatg atgaactatc gaatattgtc catggtacaa acttaataaa |
| 212161 | tatttctcaa atatactcaa cgtatacact atataagaac cttagaaagg agtgagatgt |
| 212221 | gtagatgact tacacNNatg ggtagagaaa cacataaaca aattgtttca ttgcagcaaa |
| 212281 | tattggaaat catctaaatg ttcatcaaga gaggtgaaat taggccgagt gtacttgctc |
| 212341 | atgcctgtaa tcccagagct ttgggagacc aaggcaggag gactgcttga ggccaggagt |
| 212401 | tcaagactag actgagtaac atggcgagag ctcatctcca cNaaNttttt ttttNaatta |
| 212461 | gccaggcact gtcgtgtgta cctgtagtcc tagctactca gaaggctaaa gtcagaggat |
| 212521 | cacttgagcc caggagttca aggctgcagt aagctatgac acgccactgc actccagcct |
| 212581 | gggtgacaga gtgagatctt gtattgaaaa aaagaggtga aattaaataa atcatggtat |
| 212641 | gtccacacaa tgaaatgcta tgcatctgtt aaaaacaggc cagatctaat ctatgtccta |
| 212701 | ggctgggcgc agtggcttgt aatcccagcR ctttgggagg ccaagRtggg tggatcacct |
| 212761 | gagatcagga gttcaagatc agcctggcca acatggtgaa accccatctc tactaaaaaa |
| 212821 | tacaaaaaaa ttagcYgggc gtggtagcac atgcctgtaa tcccagctac tcgggaggct |
| 212881 | gaggtRggag gatcacttga acctggaagc cagtggaggt tgtggtgagc caagattgct |
| 212941 | ccactgcatt ccagcctggg caacaagagt gaaactccat ctcaaaaaaa taaaataaaa |
| 213001 | taatctatgt cctagattgt taaaaaaWWt ttaaaggtgc agaacagggc acttggaatg |
| 213061 | ctactatttg aagggagaaa aaaataaaag gacaSataaa tatttatgtt tatataacat |
| 213121 | caccagaaag atacagaaga aaaaacaaaa caggtaagtc tatactgaaa gactagggtc |
| 213181 | tgaggatgtg aaaacagggg tgacagagac ttagatttac tttttgctct caattacttt |
| 213241 | ctgtagtgtt taaatttttt accatataca ggtttttctt tttcaaaaaa acaccaccaa |
| 213301 | aaacaaaaac acagattttt ttttagaagg aaggggagtg ttatggattg agtttcttag |
| 213361 | aaggaaggag agtattatga gttgagtttt ttagaaggaa agagagtgtt atacgttgca |
| 213421 | ttatatcccc tttatatgtt gaagtcctaa cccccctaca ggttgaacat cccaaattca |
| 213481 | caaatctgaa atgcaaaatc ctccaaaatc caaaactttt tgagcatcaa catgactcat |
| 213541 | gaagatctca cccaatttcc cagaagccct cgctcccatc ggactagaat cttcttttga |
| 213601 | gaggcctttc cttttctcat ccctattgca accagtacaa aaagcaaaac agaaagacac |
| 213661 | taaagcaaaa ccaaagtttt agtcacaaaa accaaatggg ttggcgaggg aaggtggtgg |
| 213721 | ttacagggaa ggttttaagc tcaccaggcc ctgtagctga ggtttcaaaa tggcagctag |
| 213781 | ctggacgcca tctggtctaa atctgctcac ccaaacctcc ctccagttct ccaagggatg |
| 213841 | tccctaatta ctagcacagc cagctgccaa catggaatct aggtctccct tctttcaaat |
| 213901 | attagagatg tgagaccaga aagattactt cccaatacca gagaaaaggc tacgagggag |
| 213961 | gaacaaggga gagaaggaga ccttcactgc agcatgcaag gacatggcgg acgcgtccca |
| 214021 | tcccatatct cagctattat gcattcccaa ctggcttggc caacatccag agagactcca |
| 214081 | ggctggtgag tctgtgaatg tgaagtagag ataagcaagg cggcgagggg gagctcgccc |
| 214141 | actgttatat acgggctgca gcattgcgtg gcgaggagtc aggggaatct tccatttcca |
| 214201 | agacatttct gcactcgatc ctgctcccca agactaagaa ttctttMttg gcttcctccc |
| 214261 | actccctgga atgtaactaa gcagttacct tgagaacaag gaaaaattgg gggctaggga |
| 214321 | ggtgtatgag tgttttaatg tgttaaaaat cacatttacc ctggatactt gtttgcaata |
| 214381 | aaacagagag taggaaactc cattcttacc ttcatctatg ggacaggcac ccagtggcaa |
| 214441 | tcattgtgag aaatagggag aggctgatct atgcttaggg agctcagaaa atgctatgct |
| 214501 | tccttccagc tgtagggaag ctgagccact ggggaagagt ggatgcattt ggacaaacag |
| 214561 | gaatggatgg aaggctttct aggagagggg accacatgag caaagcctga agaaggggtg |
| 214621 | gttcttatct agcaatgagg aagtgtgagg agaggcaaag aaatatcttc tagaagctga |
| 214681 | gggatcaggc actggaagct tatctacctt ctccaactcc agggtaccac caaggagagc |
| 214741 | aaaatccaga aacaaggggc acctgcaaag tggtggagga aagaggagga aaggaatgtc |
| 214801 | tgccccRagc ccagccccag ggtcagatgc cgggaaaaat tcctgggatt atttggtgct |
| 214861 | gtatgagatg gatctggagc caccctggct ccaagaggga tgtgggtttg gactggggct |
| 214921 | Kccaggactg gtctggggca agctaaggac actttcacgt gtccacactt ttgtacttgc |
| 214981 | tgttcaccag tatctccttt ccccccttct cctccctgtt catggaaatc cttaaaaatc |
| 215041 | atctcctctg ggaagttctc ctgatgctct cattcagaat taacccacca ttctttatgt |
| 215101 | cgcttcaaca agttgcttgt atctcttttt ccatttacgt tttctttttc tttttttttt |
| 215161 | ttNgtttgtt tgtttgtttg ttttgagaca gagtctcact ctatcgccca ggctggagtg |
| 215221 | taatagcgcc atcttggctc actgcaacct ctgcctcccg ggttcagttc aagccctgct |
| 215281 | cctcctcagt ctcccaagtg gctgggatta caggcacctg ccatcacccc tggctaattt |
| 215341 | ttgtattttt agtagagatg gggtttcacc atgttggcca ggctggtctc gaactcccga |
| 215401 | gctcaRgtga tccgcctact tcagcctccc aaagtgctgg gattacaggc gtgagctacc |
| 215461 | acgaccagcc tacgctttcc tatatgcctt tttttttatt ttttgagatg gagtctcact |
| 215521 | ctgttgccca ggttggagtg cagtggctca atcttggctc actgaaacct ccacctcccg |
| 215581 | gattcaagcg attctcttgc ctcaacctcc caagtagctg agactacagg cgcatgccac |
| 215641 | catgcccaac taatttttgt atttttagta aagatggggt ttcaccatgt cggccaggct |
| 215701 | ggtcttgaag tcctgacctt aggtgatcca cctgtctcag cctcccaaag ttctgggatt |
| 215761 | acaggcatgg gccaccgtgc ccagcctatg ccttgtctta taattagtct ccctctcttc |
| 215821 | taaatgtaaa ctctcagatg tcaagaacta gatcttatac atctttctga ccccatcccc |
| 215881 | cagcaccagc tcttccccag agcagacact cagtaaatac ttattgaatt gaatatacag |
| 215941 | acccacaagg agcaacctgg tactggcgga catttttgat tcctgcatcc attcattcat |
| 216001 | ttgctcaaca accatgtgag tgcctaatac ctgccaacct ggctcacact ctctcacaga |
| 216061 | gcacactcRa attgtttcat caaccagcta aaagtaagtt cagtatcaat tcagggcttc |
| 216121 | ttttcagttc tagaacatag aaaatttcac aggatcacct gaacaccgca gggcgggtgc |
| 216181 | agcctacatc ccattgcctg tttctgtcca gatggaaagt cacaccctca agaaggcccc |
| 216241 | tggggaagac agattgtctg ggatgtggat gagggccccc acaggctctg cccagcccag |
| 216301 | cccagcccag cccagcccag cccacccagg ggaaccctag atacacctat cacaggagat |
| 216361 | ggcagttaag ataactctat ctacatacac agtaccctgt ggatactaag taaggagctg |
| 216421 | gtcatttgga atgaggcagg agagctctag gaagtcctct ctgcagagta gatgtggcca |
| 216481 | tggaggtggg aggattaact gaaggtcata agaaaagtct gcccttctca gagatcaata |
| 216541 | agtgcaaatt acttgagagt ccaggaactg ctctgccatg gaggaaaaat acagatgggg |
| 216601 | gcaggggcgg gaaaggaagg gggcaggtta gggacagtca ttgatctcgg cgggctcttc |
| 216661 | caagtctaaa attgtaggat tctaacctga acctcccctg ccaacaactt gaaagagttg |
| 216721 | tcttgagatc agggacatgt ggagagaaag ttgaatagca tgtcagataa taaaccaaga |
| 216781 | agaaattgtg aagtcaaaac aagggataaa ttttccttat ctctctaaga catgcatcaa |
| 216841 | agcagctgRc tgctgtctgc ctggcctggg aagatgataa tggtttttaa tggcccgtaa |
| 216901 | acaataggac cagcctttgt aataaagttt aaccaattaa tcctctaggc ttcctccctg |
| 216961 | cactctgcct actctccgct cctcctttct gtgatggggc aaccttcagg ggtctgaaaa |
| 217021 | gcaagggatc cagcccctag acgcaaacct ataggtgaca ataagagttg cccagaggaa |
| 217081 | gtgctaaaga aaattgcctc tatcctgatc tggctaaaag gaggaagaaK ccctcttttc |
| 217141 | agSgaaaagg agaggagaga tgatgcatca aggaatttgc acaagaggta aacttcattc |
| 217201 | ttattcagga gctcattcat tcattcagca aacattcatg aacacctact atgtgcaagg |
| 217261 | catctgcaga gggtgcaaaa atatacaaag aaaatacctg cccaagatgg aattaaagcc |
| 217321 | tggagggggt ggtactggga acaagacaca ttcacagatg gccgggcgcg gtggctcacg |
| 217381 | cctgtagttc cagcactttg ggaggcagac gtgggtggat cacgaagtca tgagatcaag |
| 217441 | accatcctgg caaagatggt gaaaccccgt ctctattaaa aatacaaaaa attagctggg |
| 217501 | catggtggcg cgtgcctgta gtcccagcta ctcgggaggc tgaggcagga gaatcgcttg |
| 217561 | aaactggaag gtggaggttg cagtgagcca agattgtgcc actgcactcc agcctgggca |
| 217621 | acaagagcga aactccatct caaaaaaaaa aaaaaaNgac acattcacag atatcaatcg |
| 217681 | taaaagttag tacaatagcc tctgcaaacc actgtataag tccaNcttct ttttccattt |
| 217741 | cttctttcat gtgccatgtt ccgcttgggg tggttacgac agacaatatt cagaaaatgg |
| 217801 | aagaatcaga aagagaagaa agtggaacac acaagagaaa gaaaaccaag gccctcctcc |
| 217861 | ctcctgccat ggccctctct gtcttcagcc ctagttctgg aaaagaaaag ggtgtgttgt |
| 217921 | cactagaggc ctccaggcag gtccatgcca ctgagtcaca tttcaagctg tgtttgcttc |
| 217981 | ctacaccaac aaggcatatc ttttctgaat tgtgcaatat tctggccacc tgggtttttc |
| 218041 | tacttggtat accaactcct agtactcaac aaccattccc tttactcttt tttttttttt |
| 218101 | tttttttttt tttttgagac agagtctcac tctgcacaat ctcggctcac tgcaacctcc |
| 218161 | gcctcccaag ttcaagcgat tctccctgtc tcagcctccc aagtagctgg gactacaggt |
| 218221 | gcccaccgcc atgcccagct aatttctttt ttacttttag tagagacagg gcttctccat |
| 218281 | gttggccagg ctggtctcaa actcctgacc tcaggtgatc tgcctgcctc agcctcccaa |
| 218341 | agtgctggga ttacaggtgt gagccactgc gcctggcccc cctttactct taaatacagt |
| 218401 | gtgtgccagc tgtagtagtt tatgcctgta ctccRagcat ctcaggaggc tgaggcagga |
| 218461 | ggatcacttg agcccaagag ctttaggctg tagtaagcca aggtcacacc actgcattct |
| 218521 | agcctgggca acagagcaag accctgactc ttaaaaaaaa atacaatgtg taatggtgat |
| 218581 | ataataattc aaaggttaat catctctact tttgaaaaca ataatgaata ccttgctttg |
| 218641 | catttgttta gcctcacatt tttttcagag ttcttcaaaa aatattatct catgttcatg |
| 218701 | ctacaagcaa ccttggRtgg taacaaaggt taaacatcat tatttccact ttaaattttc |
| 218761 | cctcctttct ccatttgttt tctactcaac aaacagtggt tgagtacctg cYatgtgcta |
| 218821 | agcacYatgc tgggtacagg gtcatgcaac atagcagaaa aacagcagat cMtgccttca |
| 218881 | acaggttcaY agagctatgg ggaggagaag tgaggacgtg gtccctgtct ccagggtctg |
| 218941 | gggSatcttc tagaggagaa gattgagaca agcattaaaa gattatcatg ggccaggtgt |
| 219001 | ggtgctcaca cctataatct caacactttg ggaggccaag gcaagaggat cgcttgagcc |
| 219061 | tgggaggtgg aggctgcagt gagccatgat ggtgccactg cactgcagcc tgggcaacaa |
| 219121 | agtgagacca tgtcttaaaa aaaaaaaaaa acttaatatg attttaccag gtagaaaaga |
| 219181 | gaggaaaggg gattccagag agtgggccag caatctatgc acagagaaRa gtgagccatc |
| 219241 | tggggcatcc agaccatggg gtgaggagca atgaagggca aaggctgaga aagactgaaR |
| 219301 | agggaggctg ggcccaaaca acggagccaa gagtgccttg ccaaggagtt tggactttat |
| 219361 | cctgggggca atgaggaaat cactgaaggt tttaagcaga ggtgggtgac gtgatcagat |
| 219421 | ttgtgtttca gcaagattac cctggccacg ggatggagaa agcactggac tgggaagaga |
| 219481 | ctgggcagac tctagaaaca ttcccaaaat agaattcgca ggactgggtg tcccactgga |
| 219541 | gataggagga aaaggagata atagaaacag ccttctcact tgggcttgag gggccctcgt |
| 219601 | ccaatatagg agaccagtaa gaaagccttg cccaaggtca cctcaagtta cttgatggct |
| 219661 | aactcgaatc aagaagctca agttccttct ggtagaaacc tcttagtctg cctcattcac |
| 219721 | attgatcagc aaataaattt aaccatcatc tgccagRctt tccttgtttt ccactttata |
| 219781 | catgtgcact ttttgtccta tggagattat taagatcatt agggcaaaat tccttttgac |
| 219841 | ctcccataag gcctctcaca gtacaagaca atatgtggtc aataaatatt tgttattaaa |
| 219901 | tcaaactcca ggagccccaa gaacccatca ccaggcattg ccttacttac aacatgagat |
| 219961 | atttgccttc tttaatccct aataaacagt tttttgtttt tgagacagag tcttgctctg |
| 220021 | ttgcccaggc tggagtgcag tggcacggtc tcggctcact gcaacctctg cctcccaggt |
| 220081 | tcaagtgatt ctcctgcctc agcctcccaa gtagctggga ttacaggtgc ccaccaccac |
| 220141 | gcccagctaa ttttttgtat ttttagtaga gacagggttt caccatgttg gccaagctgg |
| 220201 | tctcaaactc ctgaccttgt gatctgcctg cctcagcctc ccaaagtgct gggattacag |
| 220261 | acgtgagcca ctgcatctgg ccaacagtat tttttattta attcctagat aaagtaattc |
| 220321 | ctagatgctt gaatttcaca atccaagata attttttttt gagacagagt ctcgctctgt |
| 220381 | cacccaggct ggagtgcagt ggcgtgatct ctgctcattg caacctcacc tcccaggttc |
| 220441 | aagtgattct cctgcctcag cctcccaagt agctgggatt acaggcgcct gctaccacac |
| 220501 | ccagctaact tttttgtatt ttttagtaga gaYggggttt caccatgttg accaggctgg |
| 220561 | tttcaaactc ctgatctcaa gtgatccgcc cacctcggct tcccaaagtg ttaggattac |
| 220621 | aggcgtgagc caccatgccc agccacagtc caagatattt ctaacaacaa caacaaaatt |
| 220681 | ggagcgttca atgtggtctt gccaactttt gttttaacac cattgtttct taaaagaaag |
| 220741 | aacattgaat acatatcccc aaagtcagga taatataatt ttaagatgaa ttttccaaat |
| 220801 | taaatcagag ttcctcaacc tcagaccaat taacactgga ctgaagaatt ctttgctatg |
| 220861 | gcagtggagg ggaaggagaa gggggagctg tgggcaaaat tgcaccagtt cagaaccact |
| 220921 | gaatttttta aagtacgctt tcttaaaaat ccagttacac tgtccttata ttttggatca |
| 220981 | gtgaaaacta tcacagactg ccaccagtcc ataaacagct tttgggaaca agagactcag |
| 221041 | aggtgaagaa tgtggaattt gaaatcagac ttgaactcaa atcccagttc cacctctcct |
| 221101 | cattccctga ccttgagcaa ggcactgttc caagcctccc tttcctcccc tgcacagcga |
| 221161 | ggacaatacc accccatccc agttgtgggg aggattcaat ggaatagatg tgacgcactt |
| 221221 | agcatgtgac catcgggaac ggaagtatga cattattaac attatttcta ccacttctat |
| 221281 | aacatatgtg gtatctaata caaattaatg actgctaccc aaatttcaga ctttctgggt |
| 221341 | caaaaggacc ttacacataa tatagtggac tttcaataaa cacttaccaa atggacaaat |
| 221401 | gaaccccttg tcaccccgat ctcactagtt ccttccctga aacccgacac atctgagtcc |
| 221461 | ttttctcctt tactaaccct ttctccaatc ctgctcatgg gaattaaagc tgtaaaataa |
| 221521 | gcctggcgca cctYgggcct ctgccctggg ctctgtgggt gggagcactg tggaagccgt |
| 221581 | atcaatcgcc cccacctatg agagcctttc ttcagggcca gccatgaacg tYccccatgt |
| 221641 | catcagcatc ttcaggctac tgctgtcctt cttggatatt taacctggag gcgggccagg |
| 221701 | gacagaaaaa ggaggtggca agatccttga acaaaaggag ctataaaagg gcgttggggg |
| 221761 | aagcaaggca aacggcagat taaacaagca ggcacctcaa ggaaacgtga cgcgggaggg |
| 221821 | gattccgagc ctctctgtct gcttacttga cagcaagcag gtcagagttc ctgccatttc |
| 221881 | cttctagtag aaaccccacc aagaccacct ccccgccttg gcaggcaaag cactggtagc |
| 221941 | tctcccaagg gaaaaaaaaa acaaaaaaca cacactcctt gttacaaacc agacacagtc |
| 222001 | ctggacagag cccagctaac aataaatttc tggggttcat ctcacaagta cattaagtat |
| 222061 | cttctgacaa tctcccaaag ggctgggttt ttctggccat cacagggctg tagaatattg |
| 222121 | agtacaagga gagggcttca agaacagggg atccaagccc tcgtttcatt gatggggaaa |
| 222181 | ctgaggctca gagaggggga aggaacttgt tcaggatcat ccaagtatgc tccYgtcagt |
| 222241 | Rgcagcactg agccaagaac tcaaggcttc agttttggca ctgggttttg ccagaggctt |
| 222301 | gaaaacatac caagaagtgg aaggtgtact atgagctaaa tctcactttt cctgaaaata |
| 222361 | tagatgtgtg ctcacacagg cacacatgtg cataaaaggt agggaaagag gaaaacaaat |
| 222421 | gtagcaaaat gctagtaaat ggtgaatcta agcaaagRgg atattggtgt tcattgctct |
| 222481 | attttcacaa cttttcagaa ggcttgccat ttttccaaga aaaatctgag gaagggctag |
| 222541 | gtgtggtggc tcatgcctgt aatcccagca ctttgggaag ccaaggtggg aggatcgctt |
| 222601 | gaggtcaaga atttgagacc agcctgggca acataacaag accccatctc aacaaaaaaa |
| 222661 | ttaaaaattg ccaggcatgg tggctcacac ctgtagtccc agctactcag taggctgaag |
| 222721 | caggaggatc gcttKagccc aggagttcRa ggctgcaatg agctatgatt gcactactgc |
| 222781 | actctagcct gggcaacaga gtaagaccat gtttctttaa aaaaaattaa aattaaaaaa |
| 222841 | taaaaaattt tgagggagaa agataaagca ctaactagca atctaagata aaaaattttt |
| 222901 | ttaaaaaaat cacttggaaa agagtaggaa ttcctccttc aatttcattc agtttcccta |
| 222961 | gctagggact aatatcctta gaaaaagtta taagaaatcc agaggttaaa ggggtgggac |
| 223021 | tccaaacaag taagagatcg ccagtattca tagcagtccg cagctacagc aatcttagaa |
| 223081 | tccaaatacg atttcagata ttttgtccca tcagctccta aaatacatat attctggtca |
| 223141 | caccatgtat cctctgttgt ttttggtgca aaaagtaaag taaccaYgaa ctacagatag |
| 223201 | ggcaaatgtc agaatttttc caggggactt tatcaccttt gcaatacagg tagtttataa |
| 223261 | atgcaataca atatagttta atttctattc aatttgcaaa ttctttcaca tgagtaaatc |
| 223321 | atttaatcaa aatccacatc caacccacac tcaccatgtt ctctatccat attagtattt |
| 223381 | ggtttgggcc atactgggtc cataatccag ccctgaaaat ctatcctggg ccacatatat |
| 223441 | aactcatacc aatagcaggg cactcaaggc atgaagattt gagggaacac aacctatcca |
| 223501 | agaaacctca aggaattcct tatggctgaa gtaaggtctg aggaaaccat gagcagggga |
| 223561 | atatgacttg gagagaaggt agaggttaaa tccaggaggg ctcatacacc caacaaagga |
| 223621 | gttggaagtt tatccttcag aaaacaagga gtcactgaag ggttttaagt aggcagtgat |
| 223681 | ataattggat cattctggca gagtacagat ggatgaaagt aactcaaagc taggaagact |
| 223741 | atcggaatga caaagacctg aactaaggca gtctgagcgg gagtgaactg ataacacatt |
| 223801 | aggaagagct attgaccaca gcaaatggac aaaactgggt gggaagaggg cagatgtgtc |
| 223861 | tgagagagac agaaaggatc ccagatgacg tctagcttgg ccaactaggt agacgatgac |
| 223921 | aacactcacc ctgacgggga atccgggagg aggcgaaggc ttggcagagg aacgtggtga |
| 223981 | gttttgaacg tgttgaggtc ctggttggat atacaaatat ggagctccag aaagaagccc |
| 224041 | aggatgaata tgtgcattgg gagtaaccaa cattaatgta cggggatgga tgccaagggt |
| 224101 | gcagaggagg tcagccagag aattgggggc aaaacaagaa ctgagtcaag gacagaaact |
| 224161 | ctgggggaaa aaaaaaattt aagggcagtc agggaagaag agaccccaaa aaacgttgag |
| 224221 | gagtagccaa agaggtaaag acctaggcct gcttttaagt gacccactaa actcctccat |
| 224281 | ctccacaggc tggtcacacc atgctggggc tggggaagga ataatcatgt gttaaaataa |
| 224341 | actcatttct caatgaaaag acaaacagcc caatttaaaa atgggcaaat aacaaataga |
| 224401 | catttctcca cagaaaacat acaaatggcc aatagacaca tgaaaacata ggaagtatca |
| 224461 | ttcggtcatt agggaaatgc aaatcaaaac cacaatgagg ccgggcatgg aagctcacgc |
| 224521 | ctgtaatccc agcactttgg gaggtcgagg caggtggatc acttgaggcc aggagttcga |
| 224581 | aaccagcctg gctaacatgg tgaaactcct tctctactaa aaatacaaaa attagccagg |
| 224641 | tgtgctggtg cacgcctgta atcccagctg ctcaagaggc tgaggcacga gaattgaacc |
| 224701 | tgggaggaga aggttgccgt gagccgagac tgcgccattg cactccagcc cgagtgacag |
| 224761 | agtgagactc tgtctcaaaa caaacaaaca aataaacaaa aaacccacaa tgaggccagg |
| 224821 | catggagtct gacgcctgta acgccaacac tttgggaggc caaggcgggt ggattacttg |
| 224881 | aggtcaggag tttgagacca gcctggccaa tatggtgaaa tcccatctct actaaaaata |
| 224941 | caaaaattag ctgggcatgg tggcaggcat ctgtaatccc agctattcgg gaggctgagg |
| 225001 | caagagaatc acttgaaccg gggaggcaga ggttgcagtg agccgagatc gtgccactgt |
| 225061 | actccagcct gggtaacaga gtgagactgt ctcaaaaaaa aaaaaaaaaa aaaaatcaga |
| 225121 | taccacttca cacctggtgg gatggctaga gtcacaaaga ggtagaggaa ctggaccccc |
| 225181 | catacatcac tgattggaac acaaaatgcc acagtcactg tggaaaacag cttggcagtt |
| 225241 | cctcaaaact tcaaacacag agttattata tgacccagaa actcctcccc taggtatata |
| 225301 | cccaaaagaa ttgaaagcat atgtccacgc caagacttgt acatgaaagt ttgtgttagc |
| 225361 | attcttcata acagccaaaa agcagagata aagcaaatgt ccatcaacag atgaataggt |
| 225421 | aaatcaaatg tggcgtattc atacaaggga ctattattca gccataaaaa ggaagggagt |
| 225481 | actgaaacat gcaacaacat ggctgaacgc tgaaaacatt atgctgagtg acagaagcca |
| 225541 | gatacaaagg ccacatgtgt tatatgattt catctctatg gaatatctag aatagggaaa |
| 225601 | tccaaggaga caaaaagcag attagtggtt accaggggta gaggggacag gggagtgggg |
| 225661 | agtgtctgct tcctgagtgc agaatttctt ttttcttttt ctgtcaccca ggctggagtg |
| 225721 | cagtggtata gtctaggctc actgcaacca ccgcctcccg ggttcaagca attctcctgc |
| 225781 | ctcagcgtcc tgagtagctg ggattacagg tgcacgccac catgccccgc taatgtttgt |
| 225841 | atttttagta gagacgggat ttcaccgtgt tggccaggct ggtctcaaac ccctgacctc |
| 225901 | gtgatcctcc ctcctcggcc tcccaaagtg ctggaattac aggcatgagg ccaccatgcc |
| 225961 | aggccaggat ttctttgtag ggtgatgaaa atgttccggc atcagatgtg gatgatggcg |
| 226021 | gatgtactaa ccaccactga attacgcact ttcaaatggt tagaatggtg aattttatgt |
| 226081 | tgtgtgaatt ttctctcaac agaaaaattc aatcatcctt tacaaagtga cccacagcca |
| 226141 | caaggagctg gggaagcagt ttctccagct tgcagcagga gctccagata ttctgagtaa |
| 226201 | agaagccaaa cagcctgctt cccgggagac ccttaaatgt cttcacacat ccacagctgg |
| 226261 | gggaggagac gctgatgaac aggagaagat ggagcatcgg cctcccctgg ctactcactc |
| 226321 | tgtgctcggg ccgcctcgcc cacttgtaaa accccaaatg ccagggtggc agcaaggcat |
| 226381 | acatgcaact atgacttgac ccgcagcgag gcagcaacca ggtctcacct gggcatagtt |
| 226441 | accccacaag gacaggaaag gagaaatggt ttccaggccc aggacaccca gaagccacca |
| 226501 | aaaaccaacc tcatggttca tcccccttat ttgactcttc gacacctaaa acctgtgcgt |
| 226561 | tgtgttcaag atacacccgc cggagaccac attcccagac atcccgaggg ccagccgtca |
| 226621 | ccacaSgggt gcctcgggga gatgctggag acacaagtgg ctttgaaatt agcacaactt |
| 226681 | acatggcctc caggtctgct gtggaaatcc aggtccaaag aagaagccag tgaggaacag |
| 226741 | gaggctgatc attgtgttct cagaaggggt tggagcctct ggggaggggg cactgaggac |
| 226801 | agagcacggt gttctgcaga tcatatcaca tctctacttc cctctccagt gatatgcttc |
| 226861 | cctcRctctc ccattttgct ccctctctcc tgccaaactc ccagggcaac cctggataat |
| 226921 | aaagtgaccg aatgtgaatg cttccaggtg gcacaccagc actccagaga aactgtcaga |
| 226981 | gcagggggca aacccctggc ctactccagc cactggataa gcgcaccctc ctccatgcct |
| 227041 | ccctgctcct gcctgccctg aagggagaag gagcgcaatg aaactaggtc ttcccttggg |
| 227101 | gttctgacca cctcgtgggg agattatgat tgccctgtgg ttgccatgcc tactgtgatg |
| 227161 | tcactccttt tatcagtcta tctggatgaa tttttccccc ttttctctga aggaaacctc |
| 227221 | ctacccagtg cttaggagcc cattgggagc tcccgccctt gtcttatctc gatctgggac |
| 227281 | ctcagaagac tccacattcc taacaggact agctctcagg atgaagaaaa tctctctcaa |
| 227341 | gctggtcaat ctactcctag atttaagcag aaccaaacag aacctatttc aacatcccct |
| 227401 | ctcagaatgc aaacaggaga cggctgtgtg gaaagatcca atcaccttta cttaattggt |
| 227461 | cagaataaaa tcactttttt ctttttaagg cagagtttcc ttctgtggcc cacactggag |
| 227521 | tgcactggcg caatctcagc tcactgcMac ctccacctcc caggttcaag caattctcct |
| 227581 | gcctcagcct cctgagtagc tggaattaca ggcgcgcacc accacacctg gctaattttt |
| 227641 | gtgtttttta gtagagacag ggtttcacca tgttggccag actggtctcR aattcctgac |
| 227701 | ctcaagtgat ccacccgtct cggccaccca aagtgctggg attacaggca tgaaccacca |
| 227761 | cacctggcca gaatcacttt ttcagtaaaa taagtgcaaa gaagaatgag ggtgKgggaa |
| 227821 | aggtttactg atcccacact ttgtcatgca cagaggaagg caaagaaaga aagaaaactc |
| 227881 | tttcctaaat gatcacttgt ctccagcagg tgtatcttga ccattccagc catggatcta |
| 227941 | tagggagttt catgccaatg aaaagcattt ttttggggtc ctactatgca tcaggcactg |
| 228001 | tggcaggtac tggagactta gtcccagccc ttaaagaact aataatagga tcatggccag |
| 228061 | gcgtggtggc tcacgcctgt aatcccagca ctttgggaag ccgaggcggg tggatcatca |
| 228121 | gaggtcagga gttcgagacc agcctggcca acatggtgaa accccatctc tactaaaaat |
| 228181 | acaaaaatta gctgggcgca gtggcagtca cctgtaatcc cagctacttg ggaggctgag |
| 228241 | gcagaagaat cgcttgaacc tgggaggcag aggttgcagt gagccaagat tgtgccattg |
| 228301 | cactccagcc tgggagacag agcaagactc catctcgaaa aaataaaaaa agaaaagaaa |
| 228361 | gaaaaaaaag atcactgttc acaatcacaa catttacaca gcctccttca aaagaatttc |
| 228421 | ttctctagaa agataagctg agagaccaga cagttYgtct gcggccttca tgaagtgaaa |
| 228481 | ggaggcagag aagaaaggag ttggccatgg acaggttttg ccccagatcc ttgagtgaag |
| 228541 | accccagcct gctgcaagct ttttcctcat tctgtcctcc cctcctggaa tctgtggcag |
| 228601 | cttccacagg cggtgaaaga aggcagcaag cagtggctcc agtgccagct gcagcaacca |
| 228661 | caccagggga atggggccat ctgacacaga gagggtgtga gggccaggga gactgtccct |
| 228721 | gtagccagct aacccctcca gtcaagtcga ctttcacaaa atgggcctgc tcctggaagt |
| 228781 | cattgatgcc attgttaaaa cacagcacca ggccgggcac ggtggctcac gcctgtaatt |
| 228841 | ccagcactct gggaggccga gacgggcaga tcacttgagg tcaggagttc gagaccagcc |
| 228901 | tggccaacat ggtgaaactt tgtctctact aaaaatacaa aaattagctg ggcgtggtgg |
| 228961 | tgcacacctg taatctcagc tgttcagaag gctgaggcag gagaatcact taaacccagg |
| 229021 | aggcRgaggt tgccatgagt cgagatcgag ccactgcact cctgcctggg caacagagtg |
| 229081 | agacYctgtc tccaaaaaac agcaccctgg agaagacagc agtccattca cactggaaac |
| 229141 | atccagactt attacataaa aatggcaatt agtggccagg cacagtaact cacgcctgta |
| 229201 | atcccagcac tttgggaggc cgaggtggcg gtgtaatccc agctactcgg gaggctgagg |
| 229261 | caggagaatc gcttgaaccc aggaggtgaa ggttgcagtg agctgaaatc acaccactgc |
| 229321 | acttcatcct ggatgacaga acgagactcc gtctcaaaaa aaaaaaaggc aattactgtt |
| 229381 | ggcatcagca gcacagttgt tttcagccaa aacttaagac acagaactag ccttatcttt |
| 229441 | ccagtatatg gtatgtctct tgctattttt tgcaaattat ctatcatata tattatcata |
| 229501 | tatattcttt tttttttttt tttttttttt ttttttttgg agacagaatc tcgctgttgc |
| 229561 | ccaggctgga gttcagtggc atgatctcgg ctcactgcaa actctgcctc ccgggctcaa |
| 229621 | gcaatttttg cacctcagtc ccctgagtag ctgggattac aggagcacgc caccacaccc |
| 229681 | agctaatttt tgtattttta gtagagacgg ggcttcgcca tgttggccag gctggtctca |
| 229741 | aactcttggc ctcaagtgat ccatctgcct cagcctccca aagagctggg attacaggtg |
| 229801 | tgagccaccg catctggcca tatatattct tttaatggca agcgggaaca aaatcgtatt |
| 229861 | cccattttct gaatgagaaa accaagccta aaagagggac tcccaagtaa tatcaacttg |
| 229921 | caaacctatg aatttttatc ttaaatactg gctttgaggg acctttggca acagcaaagg |
| 229981 | cattcggcaa tttgtgtgga agtcacatta ccagcccacc gaataagcca caactatgag |
| 230041 | ttcttccgtg attagcgggg ctggggatgt ggcaggcagg cgtggaagtg agatccacgt |
| 230101 | gaaagaccta agcactgact ccccgaggga ctggcgaaat gacgcagggg ccagatagat |
| 230161 | gtcagatttg gccaaagtgt cctcctttct cacaagcccg tctgcctttc acctgtcctg |
| 230221 | aggtaggaca accggatgcc ctactttgct gtgcagcccc actctcttgg ggacagtagc |
| 230281 | ccaaacaggc tgctcttgct acacacagat aagaagcttt gccacccaaa accaagaaag |
| 230341 | gggccggtga ggacacacag ggaatgctca ctcagtggtg gctgagtttg tggaagtagc |
| 230401 | aaaacctgca ctgtaggacc cacacctgtg agagagggcc ccacgggaat ctcagcttgg |
| 230461 | atcaaagcag gcgggtgctc actccactga gggcagcaga gcccagctcc aggtgcgatc |
| 230521 | aggaagtgac ctgagaagct ggagttctgg actcacagac ccaggcttga atttcagcac |
| 230581 | cactcagtca ctgtgtggtc ttgagtgact tgcccttgtt tttcctcatc cataaaatag |
| 230641 | ggataaaaat acctgacctt gaaaaattat acgaatgcca gcacacagta catacgcaca |
| 230701 | gcagagacag gagctagaat tattgatttg ccaaatgcag cagtgtctgg actcagccgc |
| 230761 | ccacttctga ccaatggcct tcacttgtaa cacagctgat ctggagggta aagcctgcag |
| 230821 | ccgtggaacc aagctctcgc caaggtaact gacccatgtg gggctcagag ccttgttctt |
| 230881 | actagaacca ctctagtttg tttgattttt tttatttatt tatacacata attctgtgca |
| 230941 | ttcctttatg catatgtata catatatatg catatgtata atttattcaa atagcatcat |
| 231001 | atcactgata ctattctcct acgccctacc ctcccacatg gctactcaca gcccctgcct |
| 231061 | ttccaggcgg cccgtgttaa catcttagtt tgtatccttc cacaattctc cccatattca |
| 231121 | tgtaatttta cacacacaca cactgaatag tgaagttttg gacagtgtgg gggcgttatt |
| 231181 | gtttaacaaa aatggaatta tacatcatac acagtctctc cctctttttt tattctacca |
| 231241 | taatttgcca aaatcctttt aatggttctg ttataattca tgatataaag gtgtcaaagc |
| 231301 | tattcagcca ttcctctact gatgggaatt cactcagttt caacttttgg ccactaccaa |
| 231361 | caaaaaagca ataaattcct tgacagtatg tcctaaccaa agaccatttt ccaaccagac |
| 231421 | aaaaggccag gtcccaggat gtgagggtca cccatgaaaa ggacctcatg atgaactcct |
| 231481 | gattcagcca catcatcctt aaaattctcc ttagaagaga gaccctctgc tcctagaaat |
| 231541 | acaaattccc aggcatgggt ctggcttcca aactctagct cctccagttc ttactgcatc |
| 231601 | aagtcaattc tgcaggtctt cattttccca tctgtaaaat aaaatcataa ctgccttagg |
| 231661 | cggcaggatt tgagagtgct ttagcaaggg atttttatga gggagcagtg tgtgaaataa |
| 231721 | tatttttgct gtaattctgt gagatgttgt ttctagNttg tNtgggggtc agggaaggga |
| 231781 | gggcaaaaca gaactaacag attcaattaa gttccctctg agactcaagg tccctattta |
| 231841 | taaggtacct ggggctggat gggaaggcac ttctatcttc tgcatttctg aaacacctgg |
| 231901 | tactgtggtt gtttacaagg aaaacttagg aactggacag gggagctttg gagtgtctac |
| 231961 | ttgcccagtt ttgtcagatt ccaaagaggg gcaactataa ttgaaattat aagaagaatg |
| 232021 | gtccaatcag caaattttgg aaaatctgta atctcactgg tgtttgattt tcactcaaaa |
| 232081 | attccctaag ctctacattt cattcagtaa aaagttgggg ttaggggaga gatggagaac |
| 232141 | ttcttttgca ggggtatagt gatttatttg gtctattctt gaaaaaaaat gagaaatttt |
| 232201 | gggaaaacaa tcttaaaatt tccatttcga gtagcagttc taaaagactt tcaagagcct |
| 232261 | ctctgaaaag aagcagttac agcaacttga atgaatttac ctggttatca tatggctcac |
| 232321 | atgcatgaga gtgggggaga agacacggta aaatcaacag agttcgtgtc atgatattct |
| 232381 | gctttaaaac ccaaatcaag gccaggcaca gtggcttatg cctataatcc cagcactttg |
| 232441 | ggaggccaag ataggatgac tgcttgaggc tgggagtttg agaccagcct ggaaaatatc |
| 232501 | atgagactcc tctctctaca aaagaaattt ttttgtaatt agctgggtgt ggtggtacac |
| 232561 | ctgtaatcct agctattcag gaggctgagg taggaggatc gcttgagccc aggaggtcaa |
| 232621 | gcctgcaagg agctatgatt ataccactgc actccagcct ggatgacaga gtgagaccct |
| 232681 | gtctctaaga aaaaaaaaaa aaaaacctca aaacctgact atagaaKaat tttcttcatc |
| 232741 | aaaatcgaat caagcagata attggcaccc ctttggaaaa ggattcagca gcctctactg |
| 232801 | aacatacgcc tgccccatga cccagtaact cccctgcaag gtgtatatac ttaaggtctc |
| 232861 | caaaagacaa gtaccagcat gttcccagca ccactactca tggaagaccc agaccaaaaa |
| 232921 | ctaccacatg ctcatcaaaa gtaaaatggg aaaaKaaatc atggtgaatt tacataacag |
| 232981 | agcactatat agcgatgaga atgaatgatc tacaaccaca gccaacgctg ggtgaatctc |
| 233041 | acaaacgcaa tgttaaaggg gaaatccagc tagtaaagtg aacgtgctag aagattccgt |
| 233101 | tcgatggggt tagactcaga atagtcaccc ttggtgggat ggggtaggga gcgttggggt |
| 233161 | gcctggaagg ggcacggcta ggcttctgtg atgtttcttc atctgggggt ttgtgacaca |
| 233221 | agcatgctca acttgtgaat tttgggtggt gaacatatgt gcactttttg gtatgtatgt |
| 233281 | tatacttcag tttttaaaac tKaagaaatt agccaatgaa caaaaataaa catgattgtg |
| 233341 | agctgggcat ggtggctcac actggtaatc ccagcacttt gggaggccga ggcaggcgga |
| 233401 | tcacgaggtc aggagtttaa gaccagcctg gccaacatgg cgaaaccctg tctctactaa |
| 233461 | aaatataaaa aattagccag gcgtggtggc agacgcttgt tatcccagct actcaggagg |
| 233521 | ctgaggcaag agaatcactt gaacccagga ggcagaggtt gcagtgagcc gagatcatac |
| 233581 | cactgcactc caacccgggt gacagtgtga gactccatct caaaaaaaWa aataaataaa |
| 233641 | taaacatgat taaggataaa gcaatccagY ggacaaggct cttggagcca tattttattt |
| 233701 | gKcatgtatc attcYgggga gctctcctct gtcaatgcca agactagcta gtcgagtgtg |
| 233761 | gagaaaaggc attctgtgag aacagatgaa agggaacaga gaaactggca ctttctttgg |
| 233821 | aaaagagttg tttccaaaac ctagatgtgc agcctctacc ccaggtaatt agtccaacac |
| 233881 | actatccatt acagctgagt ccgggtttgc ttcaaaagca tcgggggttt tttgggtttt |
| 233941 | gcttccctta aaaaaaaact tgttttgaat gaggggaaaa aggcgtagtt aatttttata |
| 234001 | cagaaatctg ttaaagagat ctgatcaatg gtgacaaaat ggaagccaga gatggaaaag |
| 234061 | attcactgtg aggctctggc actgaaggcc aacttgggct tccattcagt tcaataataa |
| 234121 | tatttatcat agctgctaac aatttttttc tcttttttga gatggagttt cactctggtt |
| 234181 | gcccaggctg gagtgcaatg gcgtgacctc ggctcactgc aacctccacc tcccgagttc |
| 234241 | aagtgattct ccggcctcag cctcctgagt agctgggatt acaggcatgt gccaccatgc |
| 234301 | ccagctaatt ttttattttt agtagagatg gggtttctcc atgttggtca ggctggtctc |
| 234361 | aaactcccaa cctcaggtga tccacccgcc tcagcctccc aaagtgctgg gattacaggc |
| 234421 | gtgagccacc acacccagcc aatagctgct aagacttacg caggtttaca tatgctaggc |
| 234481 | attgctctct gagcttatat atattaactc attccatctc tatctatgag gttgggattg |
| 234541 | atataatccc cactttacac ccagagaggg taataacttg cccaaagtca cacagctaat |
| 234601 | aagtgacaaa gctaggcagt ttggctagag tctgtgctct taacaattat accatattgt |
| 234661 | ttcctagtaa gcatttctta tgcccgtact atgtgctggg caccatgctg gacaaaagga |
| 234721 | gtactgagat atataaaaca cgggtacaca cataacaggt tctagtccat gcaatgatga |
| 234781 | gaatcataca ggcattctga ggtctaattt cagaacaaaa cgttagaaaa ctgcctccac |
| 234841 | acatctggac attaagacta aaatgcagtg cctgttttct cattggtatt attctattca |
| 234901 | atagagtcaa ttttaagact aagcattcct tgacacccca cccatccttg ggtcatgaaa |
| 234961 | ctcaataaag ctctcttcat tttgattgga ggggggaaaa ttccccgtaa atatgtttgc |
| 235021 | ttctttttcc tgaatcaccc tcttgtgtga aaaggattgc ttaccactga tctaacaggg |
| 235081 | tcttgaatac agggtccctc agagggtaca agaagcccag gtgttcaggt tattaaaaaa |
| 235141 | tgaagaaatg caaagcagga ttacaacatt tttggcacat tatagatcct cacacaacaa |
| 235201 | atttacattt tcacacacac tcacgcacaa acacacatac aaagtcaaat acacaatgta |
| 235261 | aaataattat gatcacaaga ggatgatagg ctctacattt ttaaatattg actcagaatg |
| 235321 | ccctgggaat gacgtggtca aggcacagac tgtgcatttg acactttgtg atgaacatct |
| 235381 | tggtgcacat ataatctagt gcacagataa tgtcaaagtc tcattagaat cctccatgga |
| 235441 | accaggtatg gtggctcata cctgtaaatc ccagtagttt gggaggtcaa ggccagacga |
| 235501 | tcacaaccag gagttcaaga ccagcctggg caacatagtg agtccctgac tctacaacaa |
| 235561 | atttaaaaat tagccaggcg tggtggcacc cgtctgtagt ttcagctact tgaggatcac |
| 235621 | ttgagcccag gaattggagg ctgcagtgag ctataatcgt gccactgcac tccagcctgg |
| 235681 | gtgacagaga aagaccttgt ctctaaaaag aagcctccag gaatgggaaa cccaagttaa |
| 235741 | atcagctccc actccaccct aactcccttc ctaagccttc cttaagggaa aaagaacaaa |
| 235801 | atactgagcc cagtagtaag gaaagaNcag tgaaagcccc agttaggaaa ccatgaaaga |
| 235861 | gaagaaatga ggtacacagg gagaaaacgt ggatgaaaac cctagactat ggccaccaac |
| 235921 | agacccaagc tagccaggtg tagacctctg tgacacctag aggtcaagtc caagaggtca |
| 235981 | gctgaagtta cgaaaggcag gcaggaactc aatcagctac ctacttgtcc acgagctagc |
| 236041 | cctccccagg cctggcatct atgcttacct cctggcccac tcctgctggc ctctccacag |
| 236101 | gcacaggtga ggccctaaac accaggaatc ttctaatgtg tgctgccttc tcccacgtct |
| 236161 | gtcccatttc ctaggagtta atcatggtgc ccattgcaac atggcaccca atgccacttt |
| 236221 | ccagagtagt gtgacgtcca attccaagac ctggaggata aaaggtatta aagctcctgg |
| 236281 | gaccacagaa gagacagggc agaaagctgg tggaaaacca tctccaacat ctgtttcttt |
| 236341 | ttctgttaaa atctatccac tgtgtgaatt gccaaggaaa gttaagattc ctgttctgaa |
| 236401 | acgagaaggg ttctcttttc acatgacacc tgcagaagat gggaaacatg ataccccctg |
| 236461 | cagatcagag gacaccggct ccctgtaaga ggatacccca acccttgtca gtgcagcacc |
| 236521 | cggcagggcg atttttacat ttttctcctc ttgctgaagg gcctccagaa aggccaccag |
| 236581 | ggctttcccc aagagtaaaa ttctccccag ctccctagga ggtctcactg cccaccaccc |
| 236641 | ctttatttag ctaagtagca gagtttttat tttactgttt attttatttt attttatttt |
| 236701 | attttatttt attttatttt attttatttg agacagtctc cttctgtcac ccaggccgga |
| 236761 | atacaatggc atgatctcag ctcactgcaa cctctacctc ctgtgctcga gcaattctca |
| 236821 | tgtctcagcc tcccgagtag ctgggattac aggcacgtgc caccacaccc agctaatttt |
| 236881 | tatattttta gtagagatgg ggtttcacca tgttggccag gctagtcttt tttttttttt |
| 236941 | tttttttttt Ytgagacgga gtctcactct gtcgcccagg ctggagtgca gtggtgcgat |
| 237001 | ctcggctcac tgcaagctcc gcctcctggg ttcacgccat tctcctgcca cagcctttgg |
| 237061 | agtagctggg actacaggcg cccgccacca cgcccggcta atttttttgt atttttagta |
| 237121 | gagacgaggt ttcaccatgt tagccaggat ggtctcRatc tcctgacctt gtgatctgcc |
| 237181 | cacctcggcc tcccaaagtg ctgggattac aggcgtgagc caccacgccc agcctaggat |
| 237241 | agtcttgaac tcctggcctc gagtaatctg cctgcctcag cctcccaaag tgctgggatt |
| 237301 | atgagccact gcacccRgcc agtagcagag tttttaaata acttccagag ccctttactg |
| 237361 | gaRacacacc caagaagatg ggctcttatt cttcccacag aggactatag accatcaact |
| 237421 | aaattcactg gctcctactc aagctataga aagctgagga ggaccttgga acatcctctc |
| 237481 | taaattcctc ccctagttcc agtccaggta aacaaaaatt gattctattt tttcaaaaca |
| 237541 | ccagcccctt cagatggcct ggttaatctt cattaacatg ttatcttcat taacatgttt |
| 237601 | atcttcatta acatatttct cagtgataag aagggcccag ggttcaacat cccactttgc |
| 237661 | atccatgcag acaagggctg tgggaagctg taaaactgaa gaggcggtga atccaggagg |
| 237721 | gattccccag atatccgtga gttggtgttt gactttcttg aacacgtctt tcctttactt |
| 237781 | tacacttaca cttactgagc attcactctg gaccagaccg cgtcctaaga gcttcacgtg |
| 237841 | tgtggtcatt caatcctcaa accaatccta tggggtaggt actggagtta agtgatggcg |
| 237901 | ctgggatttt aacccagaag tctgattgca aagcccaact taccatgagg gccacgacat |
| 237961 | atgtctctac ttacggattc caccagtact cttgacagga gcatttacct cactatatta |
| 238021 | taagccttga tctacttttc tatctccctg aagcataatc agccataatc tgctaaccgt |
| 238081 | gcctcagttt tcattctcct ttctattttt ttttcagcgg gggcagggga gacagaatct |
| 238141 | tgctctgtca cccaggctgg agtgcagtgg ctcaatctgg gctcactaca acctccgcct |
| 238201 | cccaggttta agtgattctc ccacctcagc ctcccgagta gctgggatta caggcacgta |
| 238261 | ccaccatgcc cagctaattt ttgtattttt agtagagacg ggctttcacc atgttggcca |
| 238321 | agctagtctt aaactcctga cctcaggtga tccacccgcc tcagcctccc aaagtgctgg |
| 238381 | gattaaaggt gtgagccacc gcccagcctc attctccttt ctttattttt atgatagcct |
| 238441 | ctaccttgag tttcacccaa gcctatggcc atctgctcca aactacattt cccaacatcc |
| 238501 | cttgtggcca ggtgtggcca tgggcctaag ctaagccaat gggatatgag tggaagctcc |
| 238561 | ttaatgacaa gctacttgcc tttggatttc acctcctgct tctcacatcc tgaaatacag |
| 238621 | acttaaggat acaaaataga ctaaaacctg ggtccatgaa tgatgacttt ctggacctac |
| 238681 | cttacctgcc tggtgtgctc gccaaacgac ttatttgaca aataaataaa cttctgtctt |
| 238741 | atttagcttt tttaattttt tttatttttt tttggacaca gagtctcgct ctgtcgccca |
| 238801 | ggctggagtg caagtgcaat ggcgcgatct cggctcactg caacctctgc ctcctgggtt |
| 238861 | caagcKattc tcctgcctca gcttcccaag tagctgggat tacaagcatg caccaccatg |
| 238921 | cccggctaat ttttgtattt ttaagtagag acagggtttc actatgttgc ccaggctggc |
| 238981 | ctcaaattcc tggcttcatg tgatccgccc gccttggcct cccaaagtgc tgagattata |
| 239041 | ggcatgagcc actgtaccta gacttatgta gccattctat ttggggacct ctttgttata |
| 239101 | gcagcttagc ctgtatgctg gattcagcac ctgaagtccc acagcttgtc ttattctatt |
| 239161 | caacatctcc agaacccttt cttagggctt ggaacatagt agttgctcag taaatatttg |
| 239221 | ttagctgaat gaaaacacca ttatgtaaag caggtagtac cagggctaaa caaactgatt |
| 239281 | tactcaagct gtgcaggtgt cccgtatcca ggtgactaca atgtttcctg ctttcctgga |
| 239341 | aaacaggagt tcaggacaga gagagtaggt ttaggaaagc atttgcaata ggaaaggggt |
| 239401 | aagctttcac tgcatctgtt gttggctctc tgctgagaca acagattttg ggcttaatca |
| 239461 | aaagttagct gaggttactt cttgtctgat aaaaataatg ggctccattt gagcacttac |
| 239521 | tgtgtgccag gttctgtact aagcgcttca catacattat ggcattcatt ttcattcaat |
| 239581 | tctcacaata accctacaag gtagaaaata ttaatatctc cagatgaaca aaccaggccc |
| 239641 | ccaaaaaagt taaataactt gccctagctg catggctcgt tagtgacaaa agtaaaattc |
| 239701 | aaacccaagt tgtttgagcc taaacttact ctcttcataa ccaatctgcc actctgcttc |
| 239761 | tcttcctgtg tgacaagcca atattcccMa ctatggttat aagtttcaag ggctggacca |
| 239821 | cgttttgtgc ttatctcaat agacgccaca ggcacggagc ttccactggt ggctgtgaca |
| 239881 | cttcttctct gtgcatgatt ggtgaggggt tgcacacacc caccaggaga catgacggca |
| 239941 | gagggcaaac actctgagac ccagggtcca cccagctctc ttttgccctc tcctctccct |
| 240001 | gggctgcccc ggctgcctcc ttgacctctg cactcttctg cccacgctgc tgccgcccac |
| 240061 | atcttggaca gcatcccccg gggccgcctt cctgcctgtc tttgtgtaca attcctgcca |
| 240121 | ggcccggcag ttctcctagc taaagccggc aaggtccaga cctgcccgtY tggcctggta |
| 240181 | atggtgtggt ccacatttca cctcccacct tcaccctaac ttcatcctca tggccattct |
| 240241 | ttatccacaa caactccttc ctccccattc cctagactca gcaaagcagt caggagctag |
| 240301 | gcaaggtggc ctttatccca gaggttggga taaacctagg gctcataaat ctagggctgt |
| 240361 | gagccaaata cggcccctgg gattgctttc tggttgtgca ctatgctttt tgaaaactga |
| 240421 | gttagctagK tttaggctgg gcacagtggc tcaYgcctgt aatctcagca atttgggagg |
| 240481 | ccaacctggg tggatcacct gaggtcagga gttcaagacc agcctgacca acatggtgaa |
| 240541 | accacatctt tacttaaaaa atacaaaatt agccaggcat ggtgtcatat gcctgtaatc |
| 240601 | ccagctcctt gggaggctga ggctgaagaa tcgcttgaac ccaggaggtg gaggttgcag |
| 240661 | tgagccgaga tcataccact gcactccWgc ctaggcgaca gagagaaact ccatctaaaa |
| 240721 | aaaaaattag ctaggtttaa agtcattaga ggtcatataa aaatgcaaat ttcaaacttc |
| 240781 | tctcagaaaa tcaggagccg tgacaacact gggctcacct ccccatgtgg caaatggctt |
| 240841 | gggttgaatg tgaacatcct ccctggaagg gcataaactt cccagttgac cacagtacag |
| 240901 | tcccccaacc cccacctccc accaactccc tattcttcgt ctcctacagg tttacatggc |
| 240961 | ctgactagcc cccagaggca ttggagtttg acacgcccac agcagtgcct ccattttgcc |
| 241021 | gatgagcaaa ctgagaaaag gcaaagagaa attagtcccc cagatctcca agtgagttca |
| 241081 | tcagcaaatc cagggctggc aatcattaga caatatttat ccagctccca gatgtccccg |
| 241141 | cccctgctct gttcagtcac tgcactcatc catgtggctg ccccaatgcc ttgggcatgc |
| 241201 | cagagagaca cctcattctt tcaacacaca ggtcacaaac atcttttgtg ccatgacaat |
| 241261 | ctgattctcc tcacgtcacc ccaagattaa ctcttttcaa atctgtggtt cagattatac |
| 241321 | accctgaagc tgctgagggg tgacctctca ataaggctaa tctcccctgg acctaaatac |
| 241381 | tggtggggcg tgtgtgcatg tgtgcatgtg tgtNtgtgtg cgcRcgcgca tgtgtgtacc |
| 241441 | catgtgaact cgctcatgca tgtgcttcta gattaacact ctttcttctc attaacttcc |
| 241501 | cagacaggtg agaagtcaac atttgcccca gttagagaac cagaaagtat cagtcccact |
| 241561 | gccccatcta aaggggcagc agctgctgat ggttgccata gtggaatgaa gattcagggt |
| 241621 | tgccagatct ttcaactttt caagaagcca ggaatataac ttagaagagg ggagcaatgg |
| 241681 | tctctaaaga cctgtgctga gaaacaagtt cctaagtgct tgggagaatt tatgaggacc |
| 241741 | Rggtgctaga gaatgccggg aaatgaaYgc tcccctttga acgtcccatt ggtgaggtca |
| 241801 | gaactatgtc ctcaatccct catcagccta tcccaggact ctgaagagga ccccagggtg |
| 241861 | cttggctggc tttgccttcc agcctaagaa tgtctttgac taatattttt cttaatccct |
| 241921 | gtttgctctg tcctcaaaac aagccttttt ctctttttct tatccatctt ctccatagag |
| 241981 | ccctcaggtt cttaactcac acggcactat ctggatcttt ccagtaagta ggttgccgtt |
| 242041 | tccatttctt tgattaaaca actctaacaa agaattcatt gagatgaaaa tgcttatcat |
| 242101 | gaagcaggta acatcctttt cctccccaaa ccacaagcaa ttaacatata aatggcctgg |
| 242161 | caaagggact tctttgagag gtggcagatt ataactactc ttgctcctac acggataaag |
| 242221 | ctttttttta aagacaggaa agaacaattt acaaatgaga aaatgtcaca gatggccaca |
| 242281 | aactaacttc ccgagggatg agtattttga tgcagatact tcttcaagtg aaaaatgtac |
| 242341 | aatgtacaaa atgtccactY gaggatctgt tcctccccgt gtgggtaatc gccctcctac |
| 242401 | cacgggcaac gggtaggcaa ccgatatgcc cagaaaggcg gccaatggga ggacagctcg |
| 242461 | cccagcctcc cgattggcca ggcccgccac cagctggaac catggcgagc tttgaggaca |
| 242521 | gcagagccac ttgatgggat tctctctctc ttcaaagcat agagcaaaca tcaattaatc |
| 242581 | ctcaaaagac gctttcaaga agaacattat cccttttgga gacgcagaag ctaagaggtg |
| 242641 | ccgcaggtag gtgaagggaa ggcaggaagt tgaagaggag aggagaaagt ggagcaccgg |
| 242701 | gctttcagcc ccagttctgg ttctcctgag agagtcaagt tgcaggagaa gaatgaacct |
| 242761 | gcagaagccg ggcagggaga ggtggtggag aagccgctgc tcttgggtgc gcccctcaag |
| 242821 | gcttcccact gccccatccc ctaggagacc acggagtgaa aaaagttgga ggcctaagta |
| 242881 | cagccgtgtg tacccagccg ggcaccagag ttaagacaac tggacttgtg ctcaaaaacc |
| 242941 | cttctttagt aaaacaWccc gtaggagtct gtgggaggag gaaaaattga agtctggatg |
| 243001 | caatttgcca cactgagtct gttctggtaa ccgcccccaa cacactcaca cccccttgcc |
| 243061 | tgaaatgggg agaggggtgg gatcggtgtg tgtaaacaga ggcactgccc tgcctggctg |
| 243121 | ctctttccat cttaggagaa ccaatccaga actttctacg gctcacactc tgatcacagc |
| 243181 | ctgtcgccat gattggcaat aaacgtcaca cattctcacg ttatacattt attcgacctt |
| 243241 | taatattgtg ctctgagatt cctggaaaac ttgtatgtca ttggtcaaac gttataaaaa |
| 243301 | cagcccacta ttttttcccc actcacatcg tccgtgttga ctgccttaca caccRatttc |
| 243361 | tacatggaat gtttttagcc tgcattttaa cacttagaat aactttcttt gccacacact |
| 243421 | acatacatac tgtttgcatc aataaacaga gcccgtgcca catgcaagtt ataggtttat |
| 243481 | tggagacatt taagtgttta tagaaaggac ttttctaaag aatctgaatg ggagttctct |
| 243541 | caatttacca atagtcaaag aaataaagaa aattacccta cagctgattt tttttWaatt |
| 243601 | aaactaccaa caactattct gccatagcct tgcgtgtgtg tgtgtgtRtg tgtgtgtgtg |
| 243661 | tgtRtgcatg cttgctgagg catagaaaat atctggaagg atttacaaaa actccttagc |
| 243721 | tttggggaac agaatggaca gacagagaga aaacctcttt ttttcttgtt ttatgttctt |
| 243781 | taYtgtgcat tgtttacaat cagcatgttg ttgtttttYt ttttaagact agtcaagagc |
| 243841 | agcagtgaga aagggggaag gaaagaacaa ggagttcaat ctgtaactgt gaacaatcaa |
| 243901 | ttgagataac tcactaccct cagactagcc accatgttgc tttttaaatg tttcaaatca |
| 243961 | caaaatgaaa aagttactct aagatcatct attttttccc caggtgtggt attttgtccc |
| 244021 | catgcctgtg caaagtaagc cagtagaatt tagaaatagc ttgctgcctt tttttttttt |
| 244081 | tttttttttt tgagatagaa gcttgctcta ttaaccaggc tgcagtgcag tggcacgatc |
| 244141 | ttggctcaac acaacctctg cctcccagat tcaagtgatt ctcctgcctc agcctcccaa |
| 244201 | gtagctggta ctacaggcgc ggaccaccat gcctggctaa tttttagtag agacggggtt |
| 244261 | tcactatgtt ggccaggatg gtctcaaact cctgaccttg tgatctgcct gccttaggct |
| 244321 | cccaaagacc tgggattaca ggcatgagcc accatgcccg gcctgctacc ttttttacat |
| 244381 | acacaaaata attgcaaact ttcactgcag tactgccaac cctcatgtca ccatctacaa |
| 244441 | tgtaccacgt caagaaagga caatatcttc aaacccactt accagaagaa aaggttgaag |
| 244501 | ctgtatttgg caggatactg acatacaaac ctccaagatc tgggaaacaa ctcaggctcc |
| 244561 | ttgacttgac ttccttaacg atgacttgag aaacaccaga aagtccagaa cacataaact |
| 244621 | catgaaacca accaatagac tggaatctct cctgttatta aagtcatctt tttggccagg |
| 244681 | cgcagtgcct cacacctgta atcccagcac tttgggaggc caagatgggc ggatcacttg |
| 244741 | gggtcaggag ttcgagacca gcctggccaa catggtgaaa ccctgtctct actaaaaata |
| 244801 | caaaaattag ccaggcaagg tagtgcatgc tatagtccca gctactcttg aggctgaggc |
| 244861 | aggagaattg cttgaacccg ggaggtggag gttgcagtga gccgagatca cactctactc |
| 244921 | tctctactct actccagcct gggtgataga gtgaaatcct gtgtcaaaaa aaaagaaaaa |
| 244981 | acaaatcatc ttttattctc ttaaagaatg aatttttcat gggccatcaa cttcttcagt |
| 245041 | catttaagta tttgtttaga gaatatttca gttagaaact caggaaatgg agacctgacc |
| 245101 | tcaaagggcc ctttccagtc ataaagtaat tagaaataaa ataaaaacag gttgaagtga |
| 245161 | taagctgata agaaaatcca atttgtttcc aaaattgttc acttacagct gtttttctca |
| 245221 | tgaatatagc tatagttgtc atcagcaaat cagtggcttg gaattcgaaa ggagtgaaaa |
| 245281 | tatacatcct gggtctcttg aactcctagt gtttgccaga gctaggcagc aggatcaaat |
| 245341 | ggtcaatttc agcctggccc tgaaatcaaa accctctaac agtcacagaa gttctgcagc |
| 245401 | tagtcagtta cttactgccc acacttcact cagcacaatc cttaaagtca ccagagggag |
| 245461 | cccaaatttg cgacaaacca gaatgctttc cttttagggc atagttgtcc ttctgattct |
| 245521 | acttttgccc tctcctttcc aagactaatt tagaaacgaa gggaagtagg ctaagaaaat |
| 245581 | cctcaccttt aatattaata agacttgtgc agtccccgga tgatcagagc ccagactaca |
| 245641 | taggacttga aagaaagaat gagactttga tatggaatat gaaaaagtaa caggtaggag |
| 245701 | gggtggagag accaaactga gaggcctagg gcccagcttc tgcagctctg tcactgactg |
| 245761 | gcccRgtgac gatcacgcct atttactaag cagggatgac ctacgcttac ctgtctacat |
| 245821 | ttttatctgt gagatgaacg aaatgaaacc tccaatggtt tatttgccat gacaattttt |
| 245881 | aaaaatcagt aggaatgagg gaattaggaa ttgagcccat gctatacctt cttttcaaaa |
| 245941 | gagatttcac atgtaccatc tcatctgtat tcctggagag atagtagccc attatgagtg |
| 246001 | gctccaattt acagaaaaag taactaaggc acagagtggt gatctaccca ggacagcatg |
| 246061 | gtctgtagaa gaagcattgg tctgtttaKt gcattgaaat gtattccaga gatattggtc |
| 246121 | tgtaaagacc ttcccagaat gtgtctagaa ggttcaacca cctggtggaa gacatagaac |
| 246181 | gccttgtcat gccttcccca ttccagcctg gagttctctc catggtctca gcctgcttct |
| 246241 | atgaagctta gttttatagg gagcctttta accaagccaa cctcagagtc ccactggcca |
| 246301 | aggggatggt gggggaggca aaggattcta ctggggcagt atgtaaccca catataagtt |
| 246361 | ccatccaaag caacgcttcc aaacctagct caccaggaca ggagaaaggt taccatcagg |
| 246421 | gtactcttag ctgagaactc atacctttcg ggctttYatt ggcttgaggc atgagtgatc |
| 246481 | agcaRagccc cacatcacaa tccctgtact cagatataaa ttatattagt gggcaKggaa |
| 246541 | tgtcaggaaa cttaggttgt agaatctaga tcaacaactg cttacYgagg agctgggtga |
| 246601 | attgtaaaga aagaaagaaa caagtgttga atgactgtgg cctcattaaa catttctgtg |
| 246661 | gcctggcctg tgctttgggc tttcccagag ctgtttcatg caatagcttt gggcagggca |
| 246721 | cttctttgga cttactccaa ctacacaagg aggaggagga tgtctatggt tctttccacc |
| 246781 | ccagacacag tatcagtcac agccccacct taattccact actccaattc tgttctgagg |
| 246841 | gtatcacagg atgctgtggt gggttcccca gccccacaaa atccttttct cctctaaaac |
| 246901 | aggagaacat ctttccttcc tttctgatca tccatcttgc agccagcagg actcccagcc |
| 246961 | agaacattgt caatttactc agggatccat gaggcacctt ttggaagatg agaggggttg |
| 247021 | ggtgaaaaaa ggaagaggaa gtggggtgct ggtacttctg gaaacaagcc atttctcagc |
| 247081 | ctgctctgtg aaggtccccc aaaccctaca cagatctagg aagggaaact gcattccaag |
| 247141 | aatgcatggg ggctagaagg acccgctgga gcatggatcc cacccacagg agataatatt |
| 247201 | ttgaaaagag cacacattta accagggaag aagagcccat gccctggctt aagagcatag |
| 247261 | ctgtttcctc ctccatctgc ccccattttc ccaggcctga ctagagcaag tagttaagcc |
| 247321 | aacccaaatc cagaagtatg aataatgcgt gaagcMcagg aggacaggca gatggccttt |
| 247381 | tcacctgcgc aggctatata gctctgtgcg gaggggctga gagtccagcg cggcccctgt |
| 247441 | gcccctcagt aactgtgcaa gaggaagtgg gcctcccctg cgggggtatg aaatgtgggg |
| 247501 | cgaggactta ggagttcacc gggaagtgcc agccatcctg gggcagacac caagagctgg |
| 247561 | agtgatttgt ttWccctttt ttcatgagtt aatttgcttc cagcagattc cattagtgac |
| 247621 | ctgacagacc aagggcctgg aggaggtccc tggaagagct gctgaatcag cagactaaag |
| 247681 | gggcgaccag gaaggccaga cgtcttgcac cgagcccttt gctcagagtt tctatttgaa |
| 247741 | gagcttagaa taactatttg ctaagctctt gtcaaggcag cacctctaaa ccatgtatgg |
| 247801 | agagaagaca cacaaacctt gaccaccaaa tgtgaatggc caatcagaag ctgacacaat |
| 247861 | cagctgctga ctgaggaaaa atgattttat gggggaaaag aattcccaca tgaaataata |
| 247921 | agcaaaagtt ataaacttat taaaaagctt gttaaaaact tacttaaaaa gcaaaagttt |
| 247981 | aaacatggtt ccatctgcaa ctaggaactg gtcggggagg gagggtttga acttcctttg |
| 248041 | agtttcaatt gataatcccc gaagattaag aaaatagtga tactattact actgctgctg |
| 248101 | ctattattac aaataataat aactacaatt aacatttatt tggcacctag tacatgctag |
| 248161 | gaagtaggca atRagttttt ttgtttgttt gtttgtttgt ttttgagatg gagtctcact |
| 248221 | ctgtcaccag gttggagtgc aggggcgcaa tctcggctca ctgcaacctc cgcctcctgg |
| 248281 | gttcaagcaa ttctcctgcc tcagcctccc gagcagctgg gactacaggc acacgccacc |
| 248341 | atgcccagct aatttttttg tatttttagt agagacggcg tttcaccatg ttggccagga |
| 248401 | tggtctcaat ctcttgaccc tgtgatccgc ccacctcagc ctctcaaagt gctgggatta |
| 248461 | caggcgtgag ccaccacgcc tggcctgcaa ggagttcgtt atgcatgatt tcatttactc |
| 248521 | cttgcaacat ctctttgagg tagggaaact gtgtgggtga gaaaagtcac atagctgcta |
| 248581 | agtcatagaa aacgttcaga gcaaaatcaa acccaggtgt aggccaggtg cagtggctca |
| 248641 | tgcctgtaat cccaggactt tgggaggctc aggcaggcgg attacttgag gccaggagtt |
| 248701 | caagactagc ctgggcaaca tggcgaaacc ccatctctac caaaaatatg aaacaaatta |
| 248761 | gccaggtatg gtggcataca catgtggtcc catctactca ggaggctaag gtgggacgat |
| 248821 | tgcttgagcc tgggaggcgg aggttgcagt gagccaatat cacaccactg cactccagcc |
| 248881 | tgggtgacag agttaagact ctatctcaaa aaaaNaaaaa aaaacccagg tgtgtccaac |
| 248941 | tccaggcttg tgcacttacc cactctgcta tatggctaaa tcccacaggc tacagagaga |
| 249001 | gcagggaagt aggactcagg agaactgcct ccagccccac aggcaccctg actagctgtg |
| 249061 | ggatcttagc aagtgatttc aacttgtggg ccatcgtatt ccatcagatg attgatgtct |
| 249121 | tagggacccc ttcaattcaa accacagtta ttggtggact gcctgagtgc caggcactaa |
| 249181 | gatagggggt gggggaatgt ataaacaaaa ctaaggcgtg gtccttccct ccaggagttt |
| 249241 | acagtctagc cctgccttat gcaaaagcca aagagcctta ggtgtccctt caggtctaaa |
| 249301 | atactactca gagattcctt ttgaacatgc aaagtatttc tgaYagcatt catattcatc |
| 249361 | ttctatttgt tctgtgtgga catttgcatg gagaggtatc tgaaattatg ttcgtccaat |
| 249421 | ggatttgggg tgataatttt gctctcatct ttttactttt atgtgttgct tgaattttta |
| 249481 | gtaacaaata tacatcattt ctataaaaac aaaggcattt ttaaaatact gctaaataac |
| 249541 | actatatatg ggtgtatatg tatatgtata tatgcacaca catacctata tacacatata |
| 249601 | tatttaaaca gtaactggat atttaataat actgtggaat tattgttaac tcagataaga |
| 249661 | ttgtgactag gtttccagaa agaatcttta ttttttgcag atacatactg aaataattta |
| 249721 | cagataaaat gatgtctgag atttatttca aagtaatctc tagtgtgaga gggtaggagt |
| 249781 | aggctataga tgagacaacg ctggctctga gttgcaagtt gtggaagcaa tttgggtgct |
| 249841 | aggtgatggg tataagagtg ttcacgatac tacataggtt tgaaattttc catactatac |
| 249901 | agtaaaaaag gaactccttt caaggccaac agggaagcaa catttcccca aagctaagct |
| 249961 | aagcacccag ctggcagcat ttctacctgg ggtttctacc acccttggct tcctctcttc |
| 250021 | tttgactatc tatcacagtt taacttcatt ggttctctgc agcttcctcc ccccccccNN |
| 250081 | NNacaccatt cccccctaac cttctggaat cacgtgttgt atctatttgt acattccagt |
| 250141 | gcccaggcca tccctcttcg accttaccct ggcccagggg acagagcggg tgggcctgag |
| 250201 | caatgctccc acacacctcc gctcaaagtc agctgtttgc tgtagaggta gaacagcttg |
| 250261 | actactgggg gcgagggtca cagttcactc attcccccag caaatacgga gccagatgct |
| 250321 | ggggactcag cagtgaacaa gaccaaattg ctaccttcac agagttttca tgctagagaa |
| 250381 | ggagccaact ataaatgtta tctcccatcc ctcccagatt tagggccagg ggtatattca |
| 250441 | ccaagacatt cctgaatcac ctgggatctt gtcccaaata atttaaacct gaaagacatg |
| 250501 | atctagtgcc aagagctaga ataccaccca actacccatc ccaatccagt tccccattta |
| 250561 | actgacaaaa aaaaNaaaaa acagaaatgg tgtaagccca ccatgtaagt aaacataagt |
| 250621 | aaacagtggc agaggaaagg gtacccagac ttcactgtgg caggcccacc tgggaccaca |
| 250681 | ttcagaattt ttacaagctt Wgctgcgatg ttgaccaaag ttctccccac tttttttcat |
| 250741 | ttgcttgaat ttcactttta tttttattaa catcttttgt tttgttttgt tttgtttttg |
| 250801 | agacagggtc tcaccctgtt gcccaggcta gaatgcagtg gcacaatcat agctcactac |
| 250861 | aatgtccacc tcccaggctc aagtgatcct cccacctcag cctcccaagt agctaggact |
| 250921 | acaggcacaa gccaacatgc ctgtcttatt ttgttttact ttttgtagag acaatgtctc |
| 250981 | actatgttgc cgaggctatg cttgaactcc tatgctcaag cgatcctacc acctcagcct |
| 251041 | cccaaagtgc tgggattata gtcatgagcc accacacccg gccctttatt aacatcttaa |
| 251101 | atttcgtgtt ataaggcttg ttatacaaaa tagtggtcca ctgcagctta attctttttc |
| 251161 | tttttctttt tctttttttc tgagaaagag tctcactctg tcgccaaggc tggagtgcaa |
| 251221 | tggcacaatc ttggctcacc acaaacctcc acctcctggK ttcaagcaat tctcctgcct |
| 251281 | cagcctcccg agtagctgag attacaggcg tgcaccacca tgcacagcta atttttgtat |
| 251341 | ttttattaga gatggggttt caccatgttg gccaggctgg tctcgaactc ctagcctcag |
| 251401 | gtaatccacc cacctcagcc tcccaaagtg ctgggattac aggcatgagc caccacgccc |
| 251461 | ggcagcttaa ttcttttaat tgaagattgt gatatttacc tacacatctc taaataacat |
| 251521 | gtttttaact gttgtttctt gatttttcag ttttaggctt ccccaagtag gaaagagtgg |
| 251581 | atttaccctt tttgtagcct gtgctttctt atctgttctc actatacaca cacacacaca |
| 251641 | cacacacaca cacacacaca cacacacaga cacatacaca cttcctttct ctccatcccc |
| 251701 | ctatgctcca tctgtctgcc tgcctgctta ctatctatca tctatctgtc tgtctccttc |
| 251761 | ctccctccct ctctcgctca ctctgtgtat gtatgtactg gtgaatatat atttttattt |
| 251821 | ctcaatgatt accgttttta ttgctatgat tatgtaaRct ctattcagaa ctgtgacatg |
| 251881 | tagtattctc tagttacatt tcctccactg gataacttgc attcctggaa ttaataattg |
| 251941 | tcttatttaa ttcatttgct tagtattgta tgtactcacc RctaattcaR acccctagct |
| 252001 | ctccttaagt tgtgtatatt ctctctcaat atgttgagta tgtttctcac ccacttttga |
| 252061 | gggtgaattt ggtcattttg ctagcaaaca tgattgtagc agttttaaaa aacaaaactt |
| 252121 | ccctgccaga gataacttat acaccttgct aatataacca gtgacctcct atgtgagtgc |
| 252181 | ttgatcatgg aaaacattta gccatgtttg gctaatccta acaaagacta catttggtgc |
| 252241 | acgagaactt ccaaatcacc tttcttagca aaagaaagag agaaagaaaa gaaagaaaga |
| 252301 | gagagagaaa gaagaaagag agagaNagaa agaaagagaa agaaagaaag aaagaaagaa |
| 252361 | agaaagaaag aaagaaagaa agaaagaaag aaagaaagaa agaaggaaag aatttggaaa |
| 252421 | aacagcattt tcaatttttc ttactttatc acaataatgg aaatggtgtc ataaggtttt |
| 252481 | aatgcattca tcaaaataca gcatgttcta tattacttgt ggatttacca ttgttctaac |
| 252541 | aaaaaagtat gtattatatg ctttcaccca tagactgtaa gctctaagaa ggcaaaccgc |
| 252601 | ttcagcccta cttttctgaa atatcctcat cacctagcac aatgcctccc atgtaattag |
| 252661 | agctaataaa catttcttga attgaatccc aatgtgcttt aagaatggga gctcagtggc |
| 252721 | acacgaYctg agttagttcc ctgtgccttt atagcaacca gtttgcatcc aggtcccagt |
| 252781 | gaggcccact ggacagttcc ctaacccccc ctgcatgcta tcaacaatgc acctgctctt |
| 252841 | ggccatttcc acctggaagt tgaattggcc tctcaagcct tccagacgaa gtggagccca |
| 252901 | cacacacctg ctgccctcac agtcctcacc atcacaacaa tccacYgtct catcttccac |
| 252961 | ttgctcaggc caaatggatg cctctcttgt tgtcacatcc tactttcaat tccatcttaa |
| 253021 | tcccatcatc tctaccttca aaatgtactc agaatcagct acctctcacc acctttgctg |
| 253081 | ttaccacgtg gtccaagtca ctgcctcctc catctactct caactcagca gccagagtga |
| 253141 | tgctgtaaaa ccttctggta ggtcccaact ccaaggataa gccaaagtgc tcctattcct |
| 253201 | cccactccct ctcactctgc accagccaca tggcttccct tgctgttctt caaaacatca |
| 253261 | gccatgcctc cacctctgga cctttgcacc tgctgttccc tctgacttga atcctcttct |
| 253321 | cctggatgac cctcaaatgt tgcttctcag ggagcttttc ccccaacact cctggtccca |
| 253381 | actccccatt ttagttacct ccataaactt ctcaccatct cacttcttta cttagttaca |
| 253441 | ttatcatctg ggtgccccca ctagaatgca agctccatga aggcaaggat tttgcccaat |
| 253501 | ttgttcactg ctctatgtct agcacccaga atagtgtgac taattcaaca ataagtattt |
| 253561 | gttgaattaa attaatccta ggacacctgg aaatattttt atatccccat tagggaggaa |
| 253621 | aaattacaga atctccttcc agatatatta ttccatttat atatatatat atatatatat |
| 253681 | atatatatat atatatatat gtgtgtgtat acatatatgt gtgtgtgtgt gtgtgtatat |
| 253741 | atatatgtgt gtgtgtgtgt gtgtgtgtgt gtgtgtgtat ccccaggcct ttctcaagca |
| 253801 | aataaataag ctctaatgaa tttcaaaaga caaagaaatc tcatttcatt ttaacttttt |
| 253861 | ctcacccttc ccaagtcagg gaccacctcc tctcctcccc ggcccaaagc tggaaactgt |
| 253921 | gtgctcagtg gactcaggag gagttgcagg tgccaccgac ctgaaggcaa ttaccaccca |
| 253981 | caagtttcag gggaagagaa gattaccgag aaagggaaga gaaattagca catagatgag |
| 254041 | aaacacatgt ttaaaaactc cttctgaggt catgagtaag ctgattgaag ctggcaccag |
| 254101 | agtgacaaat taatttcatg accctcttgc tttcattggg aaatgtagga ttctaaaaaa |
| 254161 | aaaaaaaaaa aaagcaaccg atgtgtagaa aagggcacRt gtgtctgccc acatgcctat |
| 254221 | atagagtggt ctggtaagaa aacaagctgt tttccagtgc atttgttttg aatgtgcatt |
| 254281 | gagccaccta ctgtgtgccc agcagaccag agatagctaa ggctcaattc ctttactcaa |
| 254341 | ggaactccag caggtgagag tggctagcat agacacagcc caccagaagt cactatgcca |
| 254401 | ctgtgcaaat gttctgctgg caggaattaa gcactagggg cagctgttct gcctagaaag |
| 254461 | aagtctcggg ccttgaagtg tgcatagaca ctggaagagc tcatcaagta gagacctggg |
| 254521 | ggtcggggaa gaagtcctgg cagagaaggt cacagaagca aaggcctgga ggtgggaagc |
| 254581 | ccatggtggg atgtgattca agctcagagt ctgccaaaag gtggtggaat gggaggctga |
| 254641 | aatgtgaatg ttggtctagg gagtctggca tcctcctctg gtaagtgcgc cctggtccct |
| 254701 | ccagagagaa ttgatccctc ccctcctttg catcccaccc acccccatac acactcccca |
| 254761 | gggctcacac ccagctcagg gcctgggcac agggcatcac ctgctccctc ttcatggact |
| 254821 | gcttctcctc ccgacaaatg cctctccctc ccatccagca ggtctctgct caatcaccac |
| 254881 | cttctcagtg aagccacctc tcgcaccccc tacaagggga caaacccaca catacgtgtc |
| 254941 | cctactttgc tttattttct gccacagcat ttacacttgt gtttYtggtc agtgtcggcc |
| 255001 | tcctccactg gactgcgggc tccatgaagg cagggacttt gttttatcga ctgccatatc |
| 255061 | cccagcacct acagtgcttg gcacttagtg agagctcaat aaatatttga caaattaata |
| 255121 | aaaaatgcct gtaacacttg agtgacttgg gttgtttatt tcttcccctc ctacaagatt |
| 255181 | gaaaatggat atgaaagcaa agcttgagct ttcagctctc taccccggcc ctgtgcatgg |
| 255241 | tgctgcccag gttggcactc agtaattagc ggatggatgg atggatggat ggatggatgg |
| 255301 | atggatggat ggattctata atcWgtagga gccatcaaaa gtataaagaa actgaactgt |
| 255361 | gcaatgcctt tcctgtgggg aaaagtggcc tgatggggta gaagcactgc aggctgggag |
| 255421 | gtcaacgagg aagctcctgc cagtgtcccc acaagccaca atgtggcctg aatgggaggt |
| 255481 | gaacagagta ggggacagag tccagagaca tacccaaggt taagccaata tgactcggga |
| 255541 | ccaagtgtaa gtcaggacga ggggagggag gagccaaaga tgatgctcag atttttgctt |
| 255601 | agatgacagt gaactccaag gggaataagc agctttaagg gaagaagttc aattcattca |
| 255661 | ggatggaagt gtgagggagg acatcMaggc agtagaatgg tcaggaggag agagggaRat |
| 255721 | tgagtcacgg gtgctggctg gagtcatggg aatccactca ttcatttatc tttttgagcc |
| 255781 | catgccaggt cccaggcatt tactctgcta gagactatgg acgctgcagt gcccaagaca |
| 255841 | ggcaaggtct gtgctctcat ggaacttact ttcggagagg aaaggcagac aaaggaaaag |
| 255901 | tgagcaaaca gatccataga gataatttct gatattgtat gcactgtgaa gaaaataaaa |
| 255961 | ccaggccatg ggagcatgat gcggggacaa tattagatag gttgggccag gaagggcctt |
| 256021 | ctgaagaagt ggcttttgcc acaatgaggt agagaatggg gatgacgacg aagttcctgc |
| 256081 | ttcttggctt caattttctt gaacaaatct ggggtgaaat catcggctga gagcagagag |
| 256141 | atggaaaggg ttttcaggta aatggaaaaa cattggacta ttctctgcca taggaagaaa |
| 256201 | tggaaagggt gagaagagat tatgatggtg caccgaggac ctcactgacg gctgaaacca |
| 256261 | caggggggta aggagcccat caatgcaggg cgtgtgcaag tttcacaggt ctcatatatc |
| 256321 | Ratgaagctt tatgtccttg ccactgaatt gacatcagca ctaggccggg tgcatcggct |
| 256381 | catgcctgta atcccagccc tttgggaggc agaggcaggc agatcacttg aggtcaggag |
| 256441 | tttgagacca gcctggccaa catgatgaaa ccccgtctct actaaaaaca caaaaattag |
| 256501 | ctgggtgtgg tgatgtgcac ctgtaatccc agctacttgg gaggctgagg caggagaatc |
| 256561 | acttgaaccc agaaggcgga ggatgcagtg agccaagatc actccattgc actccagcct |
| 256621 | aggtgacaga gcaagactca gtctcagaaa aaaaataaaa aagaaagcag cactaatgcc |
| 256681 | ctggcagcaa tgtggaagct ctgtctccac tttgtatgag atgggcttgc cggcttgttg |
| 256741 | atttgatggt aatttagttt gatgggctat gagggttttc attacttggt tttaccaggc |
| 256801 | acattcccac caccacctct tccacagact gaggtcacct cagtctgttc ctgccacagc |
| 256861 | ctccacgcac aagccatcct gcaaattaat caccctcaac attcatcgtg ccaaacctta |
| 256921 | agtaactccc cagcacctgc aggatcagtg ccaaactttt cgcccaagta tttataatca |
| 256981 | ggtccttcgt gacctgtcca gcttctctct cccagtgacc tggctggtgt tctcactgct |
| 257041 | cctttctgaa cccaagctta ttcgaatgca caggctcagg tacccgcctc tgctccatcg |
| 257101 | ataccagcca tggagtcttg ggcaaatctt gggcaactcc ttggagtttt ctcatcgata |
| 257161 | aaaaagttaa tatggctggg tgtggtggct cacgcctata atcccagcac tttgggaggc |
| 257221 | cgaggagggc ggatcacctg aggttgggag tttgaaacca gcctgaccaa cacgaagaaa |
| 257281 | ccccatctct actaaaaata caaaattagc tgggcatggt ggcacatgcc tgtaatccca |
| 257341 | gctactcggg aggctgagac aggagaatcg cttgaaccca ggaggtggat gttgcagtga |
| 257401 | gcgaagtttg caccattgca ctccagcctg ggcaacaaga gcgaaactcc atccgactcg |
| 257461 | tgatccaccc gactcggcct cccaaagtgc tgggattata ggagtgagcc atcatgccta |
| 257521 | gcccactggt actcattttc taagggtatt cttttctaac cRgaccacga aatctccagg |
| 257581 | ctacctcaag ggtcttacaa ggaggaagca aaggcctagg tgcccaaata ctggtatttc |
| 257641 | aacaggacgg cttttatttt taataaaata atttttatag agatggggtc tcactgtgtt |
| 257701 | gtccgggttg ttctcaaact cccgggctcc aacaatcctc ccacctcagt ctcccaaagt |
| 257761 | gctgggatta caggtgtaac agctttttat aggagttctg cctcctacag cttcatactg |
| 257821 | gaaaatacga gcaaaaaatg ttgagcacat ctatgatgag taagggagat gagttgcact |
| 257881 | gtatggagaa cctgatccca ggcctggccc cacctttttt taactgtgag gcctgaggac |
| 257941 | tatcccaggc catccaggcc tctggtgtca cgtctagata gtgaggaaga acccccttcc |
| 258001 | atggtctcta aggtggttcc tagatctatt attccaagca ctagctctcc tatgagctgt |
| 258061 | actgttgagg ggcagagggg tcaaaagaac cttctccctc tctggccgat agatggttgg |
| 258121 | ggtactctga aagcttctct gttccctctg cccattctcc tctaaggtgt ttcattcaat |
| 258181 | agacataaaa acaagagcat cacactgcaa agtctgaaca attgaagaga tgccccgccc |
| 258241 | tccatgacta tcaaagttgg gggatggata cttgggggtt ctttatacta ttctttctac |
| 258301 | tgttgtataa gcttgatatc ttccacaata agtttgtttt ttgttttttg ttttattggg |
| 258361 | agattctcac tctgttaccc aggctggagt gcagtggcac aatctcggct cactgcaacc |
| 258421 | tctacctccc aggctcaagg gatcctccca cctcggcctc ctgtgtagct gggaccacag |
| 258481 | gtgtgtgtca ccatgcctgg ctattttttt gtattttttt ctggagacgg catttcacca |
| 258541 | tgttgcccag gctgatcttg aactcctgga ctcaagcaat gtggctgcct cggcctccca |
| 258601 | aagtgctgga ttactgagcc accacaccca gccagaagaa gtgagaataa cagcaataaa |
| 258661 | ctcacctgat tccacccttt catgtactaa ctagatatgg atcttttgag ccctcaaaaa |
| 258721 | ggtactgatc tttttttaaa ggaactacat ctgaacatcg taagattcaa tgccttttta |
| 258781 | accttgcaca ttggagaaag aggtatgaag gaagaagggc aaagggtacc tgggagtcag |
| 258841 | acRtctctct cctcccacca actccatcac tttgtaatgK tctctactag gatgcccaag |
| 258901 | ttcccagagc cctagtctcc tcaattgcat ttttgagggg cctaaaggag accaccttgc |
| 258961 | ctcaaaagca cttgttccgg ttataaccac tcccacacac atttaaagca tccctttgtt |
| 259021 | gttgcatatg cagggctggg aggaacaatg gtgctgaaaY tttccaggac atgaaaatcc |
| 259081 | gattgcatgg cctcacccct agggattttg attcagtggc tccagcggag gaggcccagg |
| 259141 | aatctacctt tccatcagtc acacaggtgc ccctcctgta ggtagtcctc agaggacacc |
| 259201 | ttgagaaaac caaaggaaaa gcagagggag gcaaactgct cagtgctacc tgggaggaaa |
| 259261 | acaatctacc tatagcatgg aaaacctcca aggagcaggc agggagtgtc catcagaatg |
| 259321 | acaggaagga gcgaggcagt taaagggaaa gcaccatcat caaagccttc tccagctaat |
| 259381 | accttgtcct tcctgcatat gctcctatct catttttatt ttctaaattt tacccagaca |
| 259441 | agcttcccca aaacccactc atcctgacct tcccgggctc catcactaaa tctccccatg |
| 259501 | gccaggggaa cttttagaaa gtgtactgga tcaagtccct ttcctaactt caatccttcc |
| 259561 | atggctgctt actgccctta ggataaagta cacagccctc aaacttctcc atcaaggccc |
| 259621 | ttcatgacca ggcctctcct gacctctctt accatctcat ccctctccag tgctgcagcc |
| 259681 | acagtggcct tccatgggtc ccccaacatg cacaggacct tggcacatgc tattccctct |
| 259741 | gctgggatca aatgtttcct cttcaaggaa attttccctc aatgccctag accagaaaag |
| 259801 | gtccccctac tatactcttc tatcttcctg gactttccaa cttaacactg atcccacatt |
| 259861 | tacttacata tttgtgtgat tatatgatta acatctgtct tcccaagtgg accataagct |
| 259921 | tcataaagat agggtctgtg ttggtttggc tcaccatcat gtacccaggg tcacagtagc |
| 259981 | tagtacattg tatgtgttca ataatacaac ataaatgccc agtgcactgt ggtctgggga |
| 260041 | gacatggatg aagaaaaaaa gtgagacgca tgcttgaaaa attcataatg caaatgacag |
| 260101 | gccagtgtga gacggtgtgc ttctcctggt catggccact gaaaatcttc tcgactgtca |
| 260161 | cacggatggc gtcactggat gccacaagat ctcaggctct tcccagtaag agcatctctc |
| 260221 | tgggactgtc cccagaggac aatgagaagg tggcttacct cctggggaat gtggggacgg |
| 260281 | aaaaagaggg ctgggagaga gagagtgcca acctcctcaa tccccttctt gttatctcct |
| 260341 | gaaaatgtag cttagtccct cactatcagg actcagagag agaggtgggg aagagaaggt |
| 260401 | cacagacaga aacgatcttc ctccctccta tctggagcca cagaaacagg catacccatg |
| 260461 | ttatctttga gcttgctttt ttttccctag agatgatggg gggtagggtg agcaggaaga |
| 260521 | ttcagagcag tcagttcaga aaacagggtg tttttctgct tggttatcag acccatgccc |
| 260581 | attcaaggga tgcaaaatag gctgaagttt agattctctg actcggctat tgagtcccat |
| 260641 | caattacaac acatggaaaa aactttggac agttaaaaat aacatagggg cctggtgttt |
| 260701 | ggaagatctg gagtttggaa tgccagcttg tcaagcacag atccctgcct accaaaaagc |
| 260761 | atgtgctggg aactcacata cacaccaacc agcacaagaa ggtcacagat gagcaaggtg |
| 260821 | cctgttacct accaccctga caacagtgtg tcaggccaac ttggaggaca aactctcagt |
| 260881 | cctcagccac tttggtctaa aggacttcca agccctcttc tagctgtgga aagggcagag |
| 260941 | ctcatccagg aaagaagtag tctccttccc cttagaggag gtagccatga atggaattgt |
| 261001 | gggctgagtc cagtcacctt acagagacac ttcacctctg tgtgctgtgt ttctacatct |
| 261061 | gtgaacttct tgttctttct cagaaggacc caatatcagc accaattaaa tagtatgcaa |
| 261121 | taagctgtgc tcctggacaa agcatgccat agataaatct ttcttgaaga ctctttaaca |
| 261181 | catttgttct atgagtaaaa caataaattg ataaaaataa taatgccata gatttctaga |
| 261241 | acttctttct tctaggaaaa ccagtgcttc tcctacttat catttcattt ttttaaagat |
| 261301 | gccagtgtaa ggaaaatatt ggctagtgtg tctcctcatt ttacagatgg aaaaacaggg |
| 261361 | tcactgcaaa cacccattaa aagggtaacg aggctgggca cagtggctca tgcctataat |
| 261421 | cccagcactt tgggaggctg aggcaggtgg atggcttgag gccaggagtc caggaccagc |
| 261481 | ctgggcaaca tggcaaaacc ctgtctctat ttgtattagt ccactttcat gctgctgata |
| 261541 | aagacatacc caagactggg aagaaaaaga ggtttaattg gacttaaagN cctcagaatc |
| 261601 | atggcaggag gtgaaaggca cttcttacat ggtggcagca agagaaaatg aggaagaagc |
| 261661 | agaagcagaa accccagata aacccgtcag atcttgtaag acttactcac tatcacgaga |
| 261721 | atagcccagg aaagaccagc ccccatgatt cgattacctc cctctgggta cctcccacaa |
| 261781 | catgtgggaa ttctgggaga tacaattcaa gttgagattt acgtggggac acagccaaac |
| 261841 | catatcattc tgcccttggc ccttccaaat atcatgtcct cacatttcaa aaccaatcat |
| 261901 | gccttcccaa aagtccccca aagtcttaat tcatttcagc attaacccaa aagtccacag |
| 261961 | tccaaagtct catctgagac aaggcaagtc ccttctgcct atattctata aagtcaaaag |
| 262021 | caagctagtt acttcctagg tatgcaattc caagtgggag aaattggcca caacaaaggg |
| 262081 | tttacagSgc ccaggtaggt ctgaaatcca gcaaggcagt caaacattaa agcttcaaaa |
| 262141 | taatctcctt tgactccagg tctcacatcc aggtcacgct gatgcaagag ctgggtcccc |
| 262201 | atggtcttgg gcagctccgc ccctgcaaac acccattaaa atggtaatga ggctgggcac |
| 262261 | agtggctcat gcctgtaatc ccagcacttt gggagggtga ggcaggccct acctccactt |
| 262321 | tgcagggtac agcctccctc ctgcStactt tcacagtctg gcattgaRtc tctgcagctt |
| 262381 | ttccaggcac atggtgcaag ctgctagtgg atctaccatt ctggaatctg gaggatggtg |
| 262441 | gccctcttct cacagctcca ctaggcagtg ccccagtagg gactctgtat ggaggctcca |
| 262501 | accccacatt tcccttctgc actgccctag cagaggttct ccctgagggc cccgcccctg |
| 262561 | cagcaaactt ttgccagggc atccaggcgt ttccatacat cttctgaaat ctagaaggaa |
| 262621 | atcccaaacc tcagttctag atacctgtgc accctcaggc tcaacaccac atagaagctg |
| 262681 | ccaacgcttg gggcttccac cctctgaagt cacagcccga gctatatgtt ggcccctttc |
| 262741 | agccacagat ggcccctgga gcatctggga cacagggcac caagtcccta ggctgcacac |
| 262801 | agcatggaga ccctgggcac ccacgaaact tcttttacct cctgggcctc caggcctgtg |
| 262861 | atgggagggg ctgccgtgaa ggtctctgac atggcctgga gacattttcc ccagggtctt |
| 262921 | gaggattaac attaggatac ttgctactta tgcaaatttc tgcagccgac ttgaatttct |
| 262981 | ccccagaaaa taggtttttc ttttctatca cctagtcagg ctgcaaattt tccaaacttt |
| 263041 | tatgctctgc ttcccttata aaactgaatg cctttaacag cacccaagtc acatcttgaa |
| 263101 | tgctttgctg cttagaaatt tcttccacca agctaccaat gactttcttc acggaattgg |
| 263161 | aaaaaactac tttaaagttc atatggaacc aaaaaagagc ccgcattgcc aaggcaatcc |
| 263221 | taagccaaaa gaacaaagct ggaggcatca tgctacctga cttcaaacta tactacaagg |
| 263281 | ctacagtaac caaaacagca tggtactggt accaaaacag agatatagac caatggaaca |
| 263341 | gaacagaggc ctcagaaata atgccgcata tctacaacta tctgatcttt gacaaacctg |
| 263401 | acaaaaacaa gcaatgggga aaggattccc tatttaataa atggtgctgg gaaaactggc |
| 263461 | tagccatatg gagaaaaatg aaactggagc ccttccttac accttatact aaaattaatt |
| 263521 | caagatggat taaagattta aatgttagac ctaaaaccat aaaaacccta gaagaaaacc |
| 263581 | taggcaatac cattcaggac ataggcatag gcaaggactt catgtctaaa acaccaaaag |
| 263641 | caatggcaac aaaagccaaa attgacaaat gggatctaaa taaactaaag agcttctgca |
| 263701 | cagcaaaaga aactaccatc agagtgaaca ggcaacctac agaatgggag aaaatttttg |
| 263761 | caatctactc atctgacaaa gggctaatat ccagaatcta caatgaactc aaacaaattt |
| 263821 | acaagaaaaa aacaaacaac cccatcaaca agtgggcRaa ggatataaac agacacttct |
| 263881 | caaaagaaga tatttatgca gccaaaagac acatgaaaca atgctcatca tcactggcca |
| 263941 | tcagagaaat gcaaatcaaa accacaatga gataccatct cacaccagtt agaaWggtga |
| 264001 | tcattaaaaa gtcaggaaac aacaggtgct ggagaggatg tggagaaata ggaacatttt |
| 264061 | ctcactgttg gtgggactgt aaactagttc aaccattgtg gaagtcagtg tggcgattcc |
| 264121 | tcagggatct agaactagaa ataccatttg acccagccat cccattactg ggtatatacc |
| 264181 | caaagggtta taaatcatgc tgctataaag acacacgcac atgtatgttt attgcggcac |
| 264241 | tattcacaat agcaaagact tggaaccaac ccaaatgtcc aacaatgata gactggatta |
| 264301 | agaaaatgtg gcacatatac accatggaat actatgtagc cattaaaaat gatgagttca |
| 264361 | tgtcccttgt agggacatgg atgaagctgg aaaccatcat tctcagcaaa ctatcacaag |
| 264421 | gacaaaaaac caaacaccac atgttctcac tcataggtgg gaactgaaca atgagaacac |
| 264481 | atggacacag gacggggaac atcacacacc ggggcctgtt gtggggtggg gggaagggga |
| 264541 | gggatagcat taggagatat acctaatgtt aaatgacgag ttaatgggtg cagcacacca |
| 264601 | acatggcaca tgtatatata tgtaacaaac ctgcatgttg tgcacatgta ccctaaaact |
| 264661 | taaagtatga taaaaaaaaa aagaatctag aaaaagaagt acaaattcaa cctaaaacaa |
| 264721 | gcaggagata ggaagcatta taaagattaa agcagaaatc aataagaaaa aaaaaaaaag |
| 264781 | aaatttcttc caccaagcca ggtggagtgg ctcacaccta taatcccagc actttgggag |
| 264841 | gccaagatgg gcagatcacg gggtcaggag ttcaagacca gcctggccaa catagtgaaa |
| 264901 | ccccgtctct attaaaaata caaaaaaatt agccaggcat ggtgatgcac acctatagtc |
| 264961 | ccagctactc gaaaggctaa ggcaggagaa tcacttgaac ccaggaggca gaggttgtgg |
| 265021 | tgagccaaga tcctgacact gcactccagc ctaggcaaca gagcaagact ccgtctccaa |
| 265081 | aaaaaaaaaa gaaaaagaaa gaaatttatt ccaccagata acctaaatca tctctctcaa |
| 265141 | gttcaaagtt tcacgaatct ctaggggagt ctctagggga gggggcaaaa tgccRccagt |
| 265201 | ctcttcgcta aaacataaca agagtcacct ttgctccagt tctctacaag ttcctcatct |
| 265261 | ccatctgaga caacctcagc ctggatttta ttatccatat cgctattagc atttgggcaa |
| 265321 | agccattcaa caagtctcta ggaagttcca aactttccca cattttcccg tctgcttctg |
| 265381 | agcctttcaa actgttccaa tctctgcctg ttgcccagtt ccaaagtcac ttccacattt |
| 265441 | tcgggtatct tttcagcaat gccccactct attggtacca attactgtat tagtccattt |
| 265501 | tcacacagct gataaagaca tacctgagac tgggaagaaa aagagtttta attggactta |
| 265561 | cagttccacg tggctgggga ggcctcggaa tcatggcagg aagtgaaaga cacttcttaY |
| 265621 | atggcagcag caagagaaaa tgaggaagaa gcaaaagctg aaacccctga taaacccacc |
| 265681 | agatcttgtg agacttattc actatcacaa aaatagcatg ggaaagactg gcctccatga |
| 265741 | ttcaattacc tccccatggg tctctcccac aacacatggg aattctggga gatcaatgca |
| 265801 | agttgagatt tgggtgagga cacagccaaa ccWtatcact actaaaaata caaaaattaa |
| 265861 | ctgggtgtgg tggtgcacac ctgtggtccc agccacttgg gaagctgggg tgggaggatt |
| 265921 | gcttgagcct gggaggtcga ggctgcagtg agccatgatt gcgccactgc attccagcct |
| 265981 | gggcaacagg gcaagaccct gtctcaaaaa aaggatattg tgtgccgact ctactaagca |
| 266041 | ctttacatac attaacttgc ttaattcttc caacaacccc ataaagacaa aaactattag |
| 266101 | tcctacttta catatgagga aactaaggca tagatgttag gaatcctacc caaggccata |
| 266161 | gggcttgaac ttgaacccaa actctctggc tccagaggct gcacccaagc cgtgggtcac |
| 266221 | acagcaactg ctcccagcct cctgctcccc accatggagt gtctgagctt acaaagtgtg |
| 266281 | caaagcttac aaagtatgca aggaacgaac cctaaggact tgtgtaaccc ccagcctccc |
| 266341 | cgtgaaaact aggcccaggg catttctgga aggaagtggc cagcacRcag gtagacagct |
| 266401 | gcagcagtgg caggggtgtc tgggtgggaa ccaggcagca ctaccatcag aataaaccac |
| 266461 | cccgatcccc ctaggaggaa acccactgtc ttagagacct gcagcctcac tctcccctat |
| 266521 | ttgaatcatg aaactaatcc tcagggcacc atgctttaag ctacacaggt tggcatttac |
| 266581 | attttcctcc ttggattgga tttatatatt ttatttacat catttattgg ttgttgttga |
| 266641 | agaagaagaa gaagaggagg aggaggaaga gaaggaggag aaggaggatt aaagtacaga |
| 266701 | aagggaagaa gtcatttggg tccactgctc aggtgggctc tgcccacaag tctgcccagc |
| 266761 | gttgcaaatc ggcccttgca gctggacaga gctggcctgt agctgacagg attgatggct |
| 266821 | cacaccagtg tgggccccac agggtgtctc cctcacccca cccctctggg tggtcccctg |
| 266881 | gaaagtatcc aaaatgcata acaaatgaaa ccaaggacgg ggtccagttc aaccccttag |
| 266941 | tgagagggaa gactggagac agacaatccc ccttgctgga ggacacagga tgaccacccc |
| 267001 | accctccccc tggagcctct cttttggacg atgaggagta tgagtctccc caaacagtga |
| 267061 | ctgccaaacc gggactctgc tcagaccaac ctggcccagg cttatttctc atttttccct |
| 267121 | tctgttcacR gacacccccc acctactccc aaccagccaa acagatctgt gtgctctcct |
| 267181 | ccatgcagtc cttcccatct ggccttcctt ccctcagaaa ggaagggtgt ccccaaagga |
| 267241 | atcgctcaaa cgcctcacgt ctttgttcac ttccctttgg tgactaaagt tccccagcct |
| 267301 | caccctcccg agcctaacca gaccccactg tccttctgaa ctcagctcct ctctgaagca |
| 267361 | tcctctgtca tcccagccac aggaactgct gtctgtcttc caatttcaag gacccctggt |
| 267421 | ggcctaggct gatcgtttag cgcagctatt caccacctgg aacctcaYgt gaagcagctt |
| 267481 | ctctcaggaa tgcatgctct gctgcctgta agctttgtcc ttttcctcct tccctctccc |
| 267541 | cactcccata ttcatgagat tgagtgggcc ctccttgggg acaaagccag aatactcttt |
| 267601 | ccttcYtttg tgccccttca gtgctttttg atcaaggtgc aattcatgga ggaaaggaga |
| 267661 | aagacgtaaa tagtgaatga aggaggggac agggatcaga aggaaaccta ggctggaggt |
| 267721 | gcaaactcag ggctctgagg gccttggggc agcttagctc ttagtcaaga aatgaaataa |
| 267781 | gcagcagaaa gcaaaagaaa aaggaaaggc tttccagcgg gctttttctt cccaaggatt |
| 267841 | tgaaaagcta cagtagagcc tggaggagag tgggctgtcc agggctgccg gctctgagtt |
| 267901 | ttggagagag gtggagatgc tgtggagctt ttatactatc cagaccagtt cctctgggca |
| 267961 | gcaaaggagg cacacaccca ggacaatgtt gggcaataaa gattattcca tcatttggga |
| 268021 | gcaggagcgc cttttgttct gcatttctca gggaatcaac acattccaag caggaaaggg |
| 268081 | atgagaacaa atgaccagca ggatggggac tgatagtatt gggtcctctt cattcagcag |
| 268141 | attataaaat aataaaacaa gcatcctact gctgcaagga tagcaagagc tagcaaggtc |
| 268201 | tcaaaaaggt ttcagaattc agactcacaa gccaggcagc ctccatctct cctctgacac |
| 268261 | tcgctgggat cttcagcccc tgagatgaca aacaatgtcc ccctggtgtg ctctggactg |
| 268321 | aggaactatg agaacttcct aaatctcata aaactataga ttagcgttca tccctgctac |
| 268381 | cctgtgcata agtgacctaa aattgaatgg tccgaacctg tttcccatcc cccagagaca |
| 268441 | tgtgtaatta gataaaatga ggacagaata tgtataagta tctgttatac tcagatggct |
| 268501 | gcccagaaag aaaatgagtg ccaggaaccg gacagccctg tgtcaccctc tgtcctgaac |
| 268561 | tgggattgag ggagacttac ctttgtgagt aggttgctag tgcaatatga cctctctgag |
| 268621 | ttaacgaata tttttctaca attcggctcc tgcagcccat atttggcaca ccatcagcat |
| 268681 | tttctctggc tttccatgtt gaaggatgtc tttcagacca ggatctgaac tgaaacttga |
| 268741 | ccctgggagt ccgtgtccac cttgggactt cctcttcctc cttcaggcct ctttctggtg |
| 268801 | gtaccctctt ccaaaaagcc cccaacacag aattatttgg aggacataca aaagttaYaa |
| 268861 | tgatacgctg gtatctctat ctctctcaat acaagagaga aaggccctca cttgagccct |
| 268921 | aggccatcct ttttgaagga tgaatgaagt gatctgctat acttggttgg gacacagagt |
| 268981 | cgtagggaaa atcattctct ctctaggcat tgatactgtg aaagtataca aaaggcattc |
| 269041 | tctcaaagaa gatgtatcca aaaacagtga ctggactaaa gctccatgga acagacagca |
| 269101 | gatgttcacc agaacccagc cttgggttta aaccattaca gtgctttgca tcttctggtc |
| 269161 | ttctctatct taaaaactca aaagaaaaaa gaRtcctcca gtataaataa agctaaagga |
| 269221 | aatcccacac acagctaagc atagaggagg cactgaataa aaatgaagtt gatttatcag |
| 269281 | tggctatcca taagccagag gttacttact cccctgaaag aaagcaaagc aaaaaggttt |
| 269341 | gtctttattt ctcaagacca aagggagaaa gccaagcttt ttaaagtgac caagaaacaa |
| 269401 | gaggctagaa tgagcgaggc tgccacatgt cataaatgca catcctacat gcaagcaagg |
| 269461 | ccaaacctgc atctgtttcc caactcctga aactgaaggc tctttgggga ggaattctgc |
| 269521 | ctttttcaga ttcctgccgt gaaggcagag aatgagaaac atgtcaactt tctagagtgc |
| 269581 | taaaagcaaa cttgggcagc agagcagggt atctgcctgc aggcttcgct cactcaacta |
| 269641 | ttccccacac gtttcctacg tgcctgctag gctaggtcct tgggacagaa aggcacatgt |
| 269701 | ggccaggctt ggtggctcat gcctgtaatc ccagcacttt gggaggccga ggcgggtgga |
| 269761 | tcactggaag tcaggagttt gagaccagcc tggccaacat ggtgaaacgc catctctact |
| 269821 | aaaaatacaa aattagctgg gtatggtggt gtgcacctat tgtcccagct actcaggagg |
| 269881 | ccgagatagg agaatcgctt gaacccagga ggtggaggtt gcagtgagcc gcaccactgc |
| 269941 | actccagcct gggtgagaca gagagagatt ccatctccaa aaaaaaaaaa Raaagaaaag |
| 270001 | aaaggcatat gtggctactg tccctgcctt ccacgccctt actgctaact aactcctgtc |
| 270061 | aattatttca gtgactgaga gagaagagta tgttcagctc tgcatggccc agggcaggtg |
| 270121 | tggccagttc tacctgaggg tcagcaaggg ctccaccagg gagagggacc acttaaacca |
| 270181 | tccagaagat gacagatgtg catcaagtgc gcccagggac agggtggagg tggaggacaa |
| 270241 | tcaaaccgcc tgcccagagg aaaccgcctg cccagaggag ccacaagcag tttgctcaga |
| 270301 | caccagggga atgtgtgaca agagtcgtct ctctgggcag ggacacagag catcccctca |
| 270361 | gactaacaag ttcacacacg tctctcacac tgaggttctt accctagagc ccatgggcct |
| 270421 | ccagggcatc cagagatggg ctcttccagg tctgtgaacc ccataaaacc ataggcagat |
| 270481 | gttcacatct atatgcgtgt tttccgggga gatgatccac aactttcata ggattcttga |
| 270541 | aagggcctgt gattgccaaa gaaaggtgaa aaagaaacac ccatcaagac tgagtagcct |
| 270601 | gcacactaga ctgtggtata gaatccagtt tctccctgtc tgtctcctgc atatggctca |
| 270661 | gtaacagaca tagtaagttt tcagaactag cagaactaaa gggatgagaa gagcattcca |
| 270721 | gtataagcag atggaaagaa attaccattg gcatcaccaa aacaaatacc tgtgtgtact |
| 270781 | gactgtgtgc caggctctgg gctagacttt aaaaaccagt atctctttcc ataaagccac |
| 270841 | atggaagagt gtcttctcag cctcctgttc ctggagccaa tattgcatat tggagtccct |
| 270901 | aaatgtggat tcaagtcacc cagagcaatt cactgcaatc gccagatcag gctcaaactt |
| 270961 | tagttggcag ttgcctagca acgagtaacc ctgaagacac atcaccaaac ccagatgcca |
| 271021 | aaaagggcat tttgtggaca agctagttct gaaaccagcc cctcataagt ctatttgtca |
| 271081 | ggcctccctg gggctgttgg ggtaaggagt agactttgca gccaaaggta ggctgagagg |
| 271141 | agaaagatag aaaggcattg ttttttgttt tttcagacac ggtctcactc tgttgcccag |
| 271201 | gctggagtgc agtgacacaa acacggctca ctgcagcctc aacctcttgg gctcgggtga |
| 271261 | tcctcccacc ttagcctccc aaataactgt gactaaaggc gcgtgccacc acacccaact |
| 271321 | agtttttcta tgggattttg ccctgttgcc caggctggct cgaactcctg ggctcaagcg |
| 271381 | acctgcccac ctaggcctcc aaaagtgctg ggattacaag tatgagccat tgcacccagc |
| 271441 | tggaaagtgt tctgctggtt gtatgttttc ccacctctag ggaataggca tgagaagggg |
| 271501 | aactgagggt taaaggagaa ggcagcctcc agtggagaag gcagcctcca gtagagaagg |
| 271561 | caattggtgc ctcttcactt cctctttcag attgtaccta tggttactct tccaagaagt |
| 271621 | tgcttaacag atgccaccca cacctaaaga cgcataaagc agcaggaaga cttgactgaa |
| 271681 | cgtacatcca gtccttccct aaagccatca tcctcctgca gctccaaaaa ccccactgaa |
| 271741 | ctatggaagg ctggggtgag caggatgata gacctcaaac tacttctaac agtgaaactg |
| 271801 | gggaagcagt ttttaaccaa ctctccaagg caactcactY ccttacagag gcatgaaaga |
| 271861 | ggcaaagaga tctcattctc taaaattcag agctgttgtt gttgttgttg ttgttgttgt |
| 271921 | tgttgttgtt gttgctgttg ttgttgtttt taacacaaag ctcagaaact ccttaagcta |
| 271981 | agaggcaggt gagactgggt ttcccaagac aaagtttcca gtccaaacca tcacataagg |
| 272041 | ccaacactgg gtattctccc tgtgtcgata gtgggaagcc tgggaagacg gccttcttcg |
| 272101 | gaatccaccg tcccttccct tgaaaggtct tgggggtagg aggaaaaggg gcaaataaat |
| 272161 | atatcagtct ggaggtgatt caggaagaac aaatgcggat gtcaaacaac aaataaggca |
| 272221 | gatataaatc caaataaaat attaaaatca ttaaagggct gctgctccct gaagattggg |
| 272281 | aaaaatgaag agagttaatt aaaagcaatc tccatgcact gctgagccgg ccagccctga |
| 272341 | taaatcaaca gctggggctg ctgtggaggg caggcaaggg gcttcaggag ggggtgggcc |
| 272401 | agctggggga agggggtctg ccactgagat ggctgtgatg gcaaattgaa cacagtgact |
| 272461 | taagtttcat gttttattaa atattttatg accactgaat aaagttattc tttggagagg |
| 272521 | gagaaaatga tgagctgggt aagtgaaggg ggggaagata aggatgaaga ggtattgtct |
| 272581 | gattcaaaac aYacttagga ctgtgacgtt ggaacctctg atatcgcccc caaaggccga |
| 272641 | agctttgggt tttttttctt tctacttgat ctgcaaaggt agctgtgtct tcgtcttcta |
| 272701 | aaatctctct acaaaacttg ggttatgttt atctgaggga ccagggaagg cagtgcagag |
| 272761 | tcagggaaag taatgaagag gaagaaaaga cagaaaagat cctcaaaatt gtcattgctg |
| 272821 | atcaaagcca gacttttaac acaaaactaa aaggtcagca gaaatggtgg gaactctttt |
| 272881 | ttctttcttt acttctttcc ttctcccttt tttctgcagc tagtttttgt gtgtaaaatg |
| 272941 | aaaaagagct ccaaattttg gggctcaatc taagtcaaaa gccagctatt tcagaagtgt |
| 273001 | tagaagaaca ggcccttctc tattctaagg ggaaaccctc cagttcttcc ttgatgaaaa |
| 273061 | tataatcaag agaaatgatt gggggaaagt gtttttttct cctccataga actgggattg |
| 273121 | gaaggggagt gtgtgcatgg ggaaacataa ggaattatgc atttggatat gcattttaat |
| 273181 | taggtggaca gaagcagccg cctgttaatt tttcttgctt gtttccactt agcattcatc |
| 273241 | acaggcagaa ctaccataga gcgctcccac gataatagat ggttctgcat tggctaagtc |
| 273301 | ctcagggaaa ataattagca ggagagaata tattctctcc cctcccactg ccccatccac |
| 273361 | cctgccctcc ccctatcacc agcaccgcgt tgccgcccSt ctccccactc ccctcaccta |
| 273421 | tagtggtttt cctaggaaaa gccgagggca gcagtcagcc tcctggtttg ctagcactta |
| 273481 | ctttgccctg ccaaaaaaca aggRtctagg agaagaaaca gacagatcag aagtctctat |
| 273541 | tggagtggaa gctcctcgat gtagggaggg agtcaggtgg ctgtgctgat tagttctcag |
| 273601 | gggcaggcag gcaatccatg ttggttgtgg gatgaaggaa ctccaaagag gatgagagag |
| 273661 | ttctcgttgt gctcaactgg cagccaaaga tgtatcgaga gttgctggtt gaggacagca |
| 273721 | ggagtaggga tgatgttttt tgcttcattt gctcttgtaa aggaaagttg cagggccaag |
| 273781 | taagtgcagt ggttttcacc tgcaatccca gcattttggg aagctgaggt aagaggatca |
| 273841 | cttgaggcca gaagttcaag accagcctgg gcagcacagt gagaccccca tctctataaa |
| 273901 | aaaattaaaa attaagtgtg gcagtacgca cctgtagtcc cagctacttg ggaagctgag |
| 273961 | gtgggagatc acttgagtca aggagtttga ggccacagtg agctgtgatg gcaccactgc |
| 274021 | actccagcct gggtgacaga gtgatcgcaY tactgcactc cagcctgggt gagagtgaga |
| 274081 | ccctatctcc aaaaaatata tgagaaaaga aaataaagaa agttgtagct ttgcagcaag |
| 274141 | actgaatctg aaccacttcc aaggctggca accaagacat gYatgtaact ctgagatctg |
| 274201 | accccaccgc aatgctctct ggtctacttt tctataaaga atctggcctg gcatggtggc |
| 274261 | ttacgcctgt aatcYcagca cgtggggagg cccaggtggg cagatcactt gaggtcagga |
| 274321 | gatcgagacc agcctggcca acatgataaa acccgatctc tactgaaaat acaaaaatta |
| 274381 | gctgggtgtg gtggcatgtg cctttaatat cagctactcc tgtgtctgaa gtacaagatc |
| 274441 | acttgaacgt gggaggcaga ggttgcagtg agccaagatc acgccactgc actccagcct |
| 274501 | aggcgacaga gtgagactct gtctcaaaaa aaaaaaaaaa agaaaaagaa aaagaatcca |
| 274561 | ttcttgccac tgacctccaa tgccaacagc tcactctcct tattcacaag cagatctaag |
| 274621 | cccctgcccc gcctccactt cttctctctc cctcagtcat ctcagttgaa ggaggagcca |
| 274681 | gagaccaggc aaggaccaaa ggcccaagtt cccagaagag caccccactc taccaaccgc |
| 274741 | gcttctgctt cctctggttt ggtgggtgaa ctcaggtcag ggctatgacc ggaaatgaaa |
| 274801 | tgagggtttc ccctgagctc acattctcac ttatttcaaa cccagacaat aggaaaatcc |
| 274861 | cactgagatc ccagggctat ttacacagcc tggatgagta gaccttgcat ttcctgagaa |
| 274921 | acaatgatta gatgtgatgg gtaatgccag aaacatttat ttttgtttta atatttaaaa |
| 274981 | taatgtttaa tatagtaggt attattgact gcctgaggag gccatgatca gctacaggaa |
| 275041 | acttttagtt ctcatagtaa cacctcacat ttgcactttc caccttctga aatgtttcca |
| 275101 | catccattat ttcatgtggg ccgctgcggg gaagccaggc tggaggaagg ggaagggagg |
| 275161 | agaagggaga cacaagctct cctggctgtt tggaaggaca ggcctgactt gctaactcct |
| 275221 | gccccccaag ctatcctgtc atcaccgtgc ttccacccac ctcctgttgt cagacatcac |
| 275281 | atctccatcc acctaggaag ccttcactcc aagcctcacc aggtaactgc tagtgctaca |
| 275341 | gcccctggaa ctgcctcaaa cccaaaccct gacccccacg aggagtccct gccctcaaag |
| 275401 | gaagggggat ataggctggg cacggtggct cacgcctgta atcccagcac tttgggaggc |
| 275461 | cgaggcgggc ggatcacgag gtcaggagat cgagaccgtc ctggctaaca cggtgaaacc |
| 275521 | ccgtctctac taaaaatact atatatatat atatatatat atatatatat tagccgggcg |
| 275581 | taaggcgggc gcctgcagtc ccagctactc gggaggctga gcccggggaa tggggaatgg |
| 275641 | cgtgaacccg ggaggtggag cttgcagtga gccgagatcg cgacactgca ctccagcctg |
| 275701 | ggcgacagag cgagactccg tctcaaaaaa aaaaaacagg aagggggata tgaggggaag |
| 275761 | gcaatccctg tggctgggga tgggagaatt gagtctctgg gttcctagtt cccagtgtgt |
| 275821 | ttatcccaga acgcaatgga ctaccaatag agggagaggc agagaggttg aattctgtaa |
| 275881 | tactattagg tttgggtttt tttgtttgtt tagtttataa ggtggagaat gcctatatta |
| 275941 | tctagaaatg tagccgctaa ttttttgggt ttgtttgttt gtttgtttgt ttgtttgttt |
| 276001 | gtttgttttt tgagataagg tcttgctctg tcacccaggc tggagtacat ggagtacagt |
| 276061 | agcgtgatca cagctcactg cagcctcaac ctcccgggct caagccatct tcctgcctta |
| 276121 | gcctcccgag tagctaggac cacaggtgta caccaccaca cccggctttt ttttttattt |
| 276181 | tttgtagcga cagggtctca tgatatgtcc aggctggtct tgaatgcctg agctcaaggg |
| 276241 | atcctcctgc ctcagcctcc caaagtgcta agattacagg catgagccta gcYtagtccc |
| 276301 | tagattttac agtccaaaaa tgaaggagac ctaggcatag tgaaaaccca aaagaaatga |
| 276361 | aaatggggac tcaaaaacag ccaaaaggtg aaacaaccca aatgcccatc ggcagttaaa |
| 276421 | caggtaaaca aaaagtgggc tatccataca atggccatag aaaggaatca agttctgact |
| 276481 | atgctacaac atggatgaac aaacattgaa gacattatgc taagtgaaat aagccagaca |
| 276541 | cagaagaaca aatattgtat gtttcccctg atatggggtg ctaagaatgc agaatggtgg |
| 276601 | ttcccaggga tggtaggaag caggggatgg aaaagtattg tttaRtgcat acaaagtttc |
| 276661 | tctccgggat gatgaaaagg gtctgggcag ctgggcacgg tggctcatgc ctataatccc |
| 276721 | agcactttgg gaagctgagg cgggtggatc atctgaggtc aggagttcga gacgagcctg |
| 276781 | gccaacatgg tgaaatgctg tctctactaa aaatacaaaa attagccagg tgtaatggcg |
| 276841 | catgcctaca gtcccagcta ctcgggagac tgagacaaga gaatcacttg aacctgggag |
| 276901 | gcggaggttg cagtgagcca agatcgcgtc actgcactcc agcctgggtg acagagcgag |
| 276961 | actccatcta aaaaaagaaa gaaagaaaag ggtctggaaa ctgatggtgg tgatggttgc |
| 277021 | acaaggtggg aggatccctt ggggccatga tttcaagacc agtctgagca acatagcaag |
| 277081 | acctccatct ctactaaaga ttagaaataa atgtacttaa tgccactgaa atgcacactt |
| 277141 | taaaatggct aaaatggtac actgtRtgtt atgtatattt tagcacaata aagaaaaatt |
| 277201 | gagggaatgc ctctctctgc tggaggtcag ggtgaccatt ttgttttctc tttaaagtct |
| 277261 | cagaatttac acaccagctc ctgtgtagac tctgatgagt tctgcaacct cctccccctt |
| 277321 | ctctttatgc ttgcaaaaaa actgccatgg aagctttcca ccaaagaaaa gcctgataat |
| 277381 | aatgtcccca ctgaaatctg agatgttctg gagacatgtc actgaagggg aggcatgaag |
| 277441 | tccagtctag tcttggctaa tacccactca ggtaggggct agttccacac aactgctggg |
| 277501 | cacctccatt tcctcagcag taacacaggt atgctagagg aagctcagtg agacatcagt |
| 277561 | acaggtccac ttacttcagc tcactaaaca tccaggtatc tgtggctgcc caaattaaaa |
| 277621 | taaaataagc actttatttt cagattaatt gggataggtg ccatggtaga ggagaaggcc |
| 277681 | agagagatca tggacaaata aagtccagac cggaggactg gctggaaaat tggttaccct |
| 277741 | tctgcctcca aacacacaca cacacacaca cacacacaca cacacacaca cacacaaaag |
| 277801 | gaagcaaaat acagatgttc tcagtgattc cagtctttta accaccaaaa actgagtgct |
| 277861 | tatccacgtc aaaataggcc aattggtctt caagttcctc attctcatca gcttctggat |
| 277921 | gggatgctag tgagaagaaa atcactgaaa aggaagagcc agaggaaaag cccacattct |
| 277981 | aaatggccca ccacctggtc ctcagcctca gaccttgaga agggatagga agttccaccc |
| 278041 | agcttagaac cccaagaagg gagcaccatg tagcaagact gaatcagaga ataagaaact |
| 278101 | gtggaagtct ccagtcttaa atgttctctt tagagccaca ctttaaaaat aagaaaaaat |
| 278161 | cagaactctg gcccctagct gtcctctctg tcctaatcca ccataataag agccagaaag |
| 278221 | ttgttggctt gaaaggaatt tggggaatca aagtacatca ccatcataaa gacaaggaat |
| 278281 | atgcggctcN aagttctgtg tcccagatca caagctagtt gcctgtagct ctggaagcgg |
| 278341 | agctcaaggc ttgggattcc tctcccagag ctttttctcc tccattactc tgaagtcact |
| 278401 | aaggctacaa aaaatcagcc actggtgaga aatgaacatg gaagaggaaa agaggttgag |
| 278461 | acgggtttca tcaatagcag gtgaaagtgt gaggatggga aaaactttct ggtggagctt |
| 278521 | ccaagtgcag atcattcaaa cagcagattt tcaaggtgaa taacaacaac taaaaaaaaa |
| 278581 | ccttttatca gcaacctgag atgatcacag tccccagatt cagagaggga ggagagggtg |
| 278641 | caaagKgggg attagacatg tgccagcatt gttccgcagg ctcagaactg aggcctggct |
| 278701 | ggtgaggact caatgcgggc tatttgaacc ccattattag gagctctgaa cctaatcctc |
| 278761 | tcacccctcc ctgcttccac acattgcccc taaattgtaa cctcttcagg gattctctcc |
| 278821 | cttcccttgc cactgcctgc cacactaata ttcacaaaca cacagcctag aaattctcca |
| 278881 | gtctgctcct cttccctctg ccccaccaac acctcctaac cactgctttt aaaactctaa |
| 278941 | tgcaaatcac aggcttcaaa aaccttccct cagtgacagt tttacgatgc tagctccatt |
| 279001 | catccatatg gtttgacaga cYgtgaaaca ccagtaaccg gaccaataag aaaaacaaat |
| 279061 | tacaaaaaca ctRcaacacc tcatctctcc ccagtcactc aaacaactag aaaactttgg |
| 279121 | aatttgcaat ggatcacaat atcagtctct cttgcagttc aacaacaatc tttgcagagt |
| 279181 | tccataccac acagtggttt tacccactcc tccatcctcc ccctacccaa ggccactctc |
| 279241 | catctttcct tgttaactac cttcatattt aaagactatt taaaattagg gccatgctta |
| 279301 | caaattcccc tgttgggtcg tagctgggca aaaaatattt cagtggttgt gggatttttt |
| 279361 | tggccagagg tttgcaacat ttggctacat ttctgttttc tagtgacttt caaaacactc |
| 279421 | aaaaaccaca aaagttactt tgcagaggtt gggataataa acaggcctgg attctggtgc |
| 279481 | ctctttatga cccagccccc atcgccacta ctgtcagcat agctgcctta gaaaactgaa |
| 279541 | acatcagcct cagtttccat tctttttctg gtgcatgatc aaaacaaaac atgctgacca |
| 279601 | tgacatttta ttttttgcat tgatctgaac agatggctct gaaacagctt tagacattat |
| 279661 | ggtctttctc agatgtatcc aaattccagc tacctgcatg attccaaaaa gaggaggatg |
| 279721 | agaggtctcc aactcagctg tctctgaatt caagcccacc ttatgtgact aagcagctcc |
| 279781 | cattcaactg gcagtcttca atgtctcccg ggaggcctgc agaccaggct attttcagag |
| 279841 | gtttaccctg tgacaatgcc tccacttggg ttctcgcaga cattaatctc catcataggt |
| 279901 | gtctcatcaa aagcagacat ttacccattg aaaaataatg tgtgctaatg aggtttggag |
| 279961 | aatttagggc ttttttatag tggcaaccag aagcagatgg agaacaggtt cttgggaatc |
| 280021 | tgttggctta caacgtccga gggtcctttt tcataagaag ctaaaacaat gtccataaaa |
| 280081 | ggcagcaagt tcacaaagtc ccggcctcaa tgctgtctac ttagtaccta cccacatccc |
| 280141 | cccaacctcg acagaaagag ctaagaggag attagaacca ggaatgtgac ccccaccMct |
| 280201 | tcccctctga tatctaaact gagtgccaca gcaagagtga ggtctggctt ctaactttca |
| 280261 | catgcacaaa accactttag attagaatac actaatacag gtcatagaat gcacaaagag |
| 280321 | atctagattt gacgttcatc tacacacatg acctgctgtt tgacttggct aagtaagttt |
| 280381 | attttcttct cccagagtag tgttcttaac aacaaaacag ggacataaac acatacctcc |
| 280441 | aagcaaggtt aactccttac aaaagaaagt ttcaatacaa tcacgtatat gtgtgtgttc |
| 280501 | acagttataa gcatgacaaa atagtttatg ggtctcttta taaataatta cttaaggtaa |
| 280561 | agccctgttg gttcatatgg aatttaataa taataatttt tttctaagta ggcaattata |
| 280621 | ataatagaca tatatgtgta taatcaagtc tggcagaagg tggcagacat tctcagttaa |
| 280681 | atgcatacca accaggattt tgataataca agagtaagtt gtttaaagta ctgattgcag |
| 280741 | atttggggga aggtggttta tctgaactag aatattaaga ttcaaattta tattaaaagc |
| 280801 | attatgtggc cagacatcgt ggctcactcc tataatccca gcactttggg aggccaaggc |
| 280861 | aggcagatca cttgaggcca cgagtttgag accagctggc caagatggag aaaccccgtc |
| 280921 | tccactaaaa aacacaaaaa ttagccaggt gtagtggtgc gcgcctgtaa tcccagctac |
| 280981 | tctggaggct aaggcacaag aatcacttga acccaggagg cagaggttgc agtgagccaa |
| 281041 | gatcctgctg ctgcactcca gcctgggcaa cagagcaaca ccctgtctca aaaaataatR |
| 281101 | ataataataa taataataat agcattatgt ttcccaggta tgcaSaaatc aaccagaacg |
| 281161 | tgactaatct acatgcaggc ctttatcaaa tccatattta tgttacctgc atgtgtttac |
| 281221 | ttcactacct gggccttatc tcgcttgtat atgtaagaaa tgagggtaac tctcatacat |
| 281281 | ctttgctcag acaatgtagc tgctaaatta aataatatgt aatacacatt tacacatcat |
| 281341 | acatgtccac cagtgctttt gaattctatt tgtccagatg tgactgcttg gttgtggggg |
| 281401 | ccacctgccg tgtccccttt gtgatctgat cagtgcaggc ggctcctatt tctgcctcta |
| 281461 | acaccctgtt gggcagtaat aagatgttaa aggcgacaac agagagagaa gctgggctgt |
| 281521 | cagcttcaga gaggaagggg gtgaagggta caacaaggaa gaaaaagaaa gggagttgag |
| 281581 | gggagatcaa agacaggcag gaagaaagcc aaattgctaa gagaggagat gtggcttcag |
| 281641 | gagcttggga ccctaaggaa aaccccaaag caatgagatt caaaattgtg ccatcccatc |
| 281701 | accccagcta attgaattat tgaatgtccc acgtggaaaa agctagtaca gtgaaaaata |
| 281761 | gcccatactc agcgtaggca gagccctgca taggtgctgc tggaaatctg agcttgtttc |
| 281821 | ttcccaacac attaactacc accactttat ccccccatcc tgaggaaacc tttgtgtgac |
| 281881 | cattttctgt aggagacgtc cgtggtgttg gctttttttt ttttggaggg aagagagaga |
| 281941 | ctcagaacgc acacagagaa aatgctaatg tcgttcagct ctgtttcatt gtcaggaggg |
| 282001 | acacatgagg ataaaaacat agctctccaa accattctcc ccgctacccc tcctccctgc |
| 282061 | accaccacca tcccttcggc tggatgtgtc tctgcacttt gttctaattt atcccatgca |
| 282121 | taatttattc acgtgtgtat tgtttcctga tcagcaactt ttaaaaacaa atgaaatata |
| 282181 | taagatgttt tattaaacaa gttattttta aaaaaataaa ggcagatggt gtccctctcc |
| 282241 | caataacaac gtgtgtgctc ctgcgttagg agttgtcaaa aactgtccag ggtgtgagag |
| 282301 | gctgggctgg tcagtggtat tgtttatacc cctttactca cagggaaagg gtctgggaag |
| 282361 | cagaaaggaa tagctaatta taaatgacca agcaactccc acctagaaaa agcaagcagg |
| 282421 | ggcgttgtgt gtatgctagt gtctgcatgt gggtggcaac acaattacaa atacatcacg |
| 282481 | ttgcaactgt ttctaattat acaaagtaca attcagaact aattccacta accggccaga |
| 282541 | attccgccac cttctttccc gcgtctgtcc cttcccccac ccttaacccc accctacaca |
| 282601 | gactccctcc ccaaacacgc accctactgt gatccaaaaa accctggagc gagtMgggga |
| 282661 | acacaagctt agagatactg cgcccgcccg cgcgaaagcg ggctcccggg taactagggt |
| 282721 | acggctttcc caggaggaaa caaacctggt gcacatgtga aataggttct ctaacttgag |
| 282781 | tttaccaggt ggcatcagcc cggttatgtg atttcggctg gaaatgggga aactgMaaat |
| 282841 | cagttgtggg ttcggtagaa acagcatcag cggtgttgca gcgaaagcaa ccggcagcga |
| 282901 | ggatgcggcg aagttacgcc catttattct gccataaaac attttctttc ttgggtgaaa |
| 282961 | cgctgtggcg ttcaagcgca gtgtatccga gcttccgagc cagaactgaa gccaggagct |
| 283021 | cctatctgat cagcctcagc ttccaacgtc ctcttaacgg cagctttcat ataaatatat |
| 283081 | ttgaaacctg gggccttcct acccctttcc ctatttctga tgggcgacga gctggcgcaa |
| 283141 | acacgtccga ttctccctgt gcattccttt gcgttaggaa ccccctaccg tccccgggag |
| 283201 | cgaacctggt tttcaactcc aggagtggcc aggggtgaag acgagaatgc gctgaattaa |
| 283261 | aagaaccagg gtcgacagcg gccacgcacc agggagaatt cggcatgaac cgccccccac |
| 283321 | ccgacctgca cctccctctg ccacttcgcc tgcggcttcg tccgggcggg gaagtcccgg |
| 283381 | tcgaaggggt gaggaccccc cagcccacct cggatacgga gggagccacc ctctgccgac |
| 283441 | atcacccaat agcacccctt tcagagcgac actagttttc cgaggccacc cctggctccg |
| 283501 | cgatcgcgag cggacttggc ggcttaacag ttccacYgac tggaaggaag gaaggaagga |
| 283561 | aggaaggaag gaaggaagga aggaaggaag gaaggaagga aggaaggaag gaaggaagga |
| 283621 | aggatgaaaa ggaggaggaa ggcaggcagg cagaaattgg ctgttggacc cggggctggc |
| 283681 | ccccgcgagt cctcgcccgg cccgccccac tctgtccagg atctgggcaa gcgctcaaag |
| 283741 | tctccccgag gcggccagag gcacctgccc ctcgcccggc cgggagcaga cgctgccccc |
| 283801 | tcgggccagg agccccaaaa ccgcctctcc gcctgcctca gcgcctcacc cagaggtggc |
| 283861 | gagaacacca ttcataagac ctgcggcttc cctgcctcca aatccacccc aagttaaccc |
| 283921 | gagtcgttaa tccactaaag ccagctcgct cgcaaataca cgagcagaca cacgcKcgat |
| 283981 | cgcccggagc gcctccgaga tccgggggct gccggggaaa gcccggggcg accacctggg |
| 284041 | ccccagcacg cccgggaccc gctgcttccc ccgcggtgct ccaactccgg aaggtttgca |
| 284101 | caaactcccc gcgagcgatc ggaaggcgaa gagcgccctg caaactccRg agcctccgcg |
| 284161 | gtccgtccgc tggacccagc ggacccagcg gcggggcggg ggccgcgggc tcgggggcgc |
| 284221 | ggggctcggc caagtacacc gaggcggcca ctgcccgacg ctcgcccccc gcccagcagc |
| 284281 | ccccgaagcc gcgctccctg cccgcgaggc gggggactcc ggggaccgcg ccgtccccga |
| 284341 | gctcccgcga ccctcctgct gtagagcgct cgagcgccgg gcttctccgc ctgcaacccc |
| 284401 | tatatctggc tctctctgca atttagcaga acacaaatgt aacaattttc ttcttgttaa |
| 284461 | ttgcatctcc tgacatttct gcgcgttggg ggaaaaaaag aagatgaaga agtaagagct |
| 284521 | gaaagaagag agaagccccg ccgcggctgg aggcgcctgc aagcgcaatg ctctggcagc |
| 284581 | gctcgccacg cagaaggcgc cttttttttc atattgaaac cacgcggaat atgtacattt |
| 284641 | tttagtctta cttacgcttt cagggggttg tgaatgacag tcgccatttt gctacaatgt |
| 284701 | aacagaatat tgtctcagag ttctaaaggt tcgctcaggg gMagcgacgg gccaatcaca |
| 284761 | gcgcgggata ccggcccgcc ggccaatcgg cgctgctggt agctaggtgg gcggggcctg |
| 284821 | cgccgtggta gttattaggR gagctgtcaa agcccagagg tcactccctt ttgtagagcc |
| 284881 | cgagagccag caggtcttaa aggggcagta cMgcgctggt agctcctttt cgggctgggg |
| 284941 | aaagtggaga cgcacgggag agtattatag tttggtttaa ctccctactt ttctaaataa |
| 285001 | ctgcagaaga gtttgcgctc ccggacagaa atatttaaat attgccagca agccactgaa |
| 285061 | tcctattcat tagcctttgg ctttgcgtat Yaacattttt aatcccccaa agggtaaatc |
| 285121 | cccgtgtttc agtgcaaaag ttggaaggct gccgtttcgt gttaacaaat tactacatgt |
| 285181 | gctctgccgt ttgctaatgc tctgggtcct gccgcttttt aatattctta ttcccgaagt |
| 285241 | gttctcttta tctcggccac cttcctctct cgcttcttgg aactgtcgct gctgttcctg |

Following is a DPF3 genomic nucleotide sequence that includes the DPF3 SNP “DPF3-AA” (SEQ ID NO: 4962).

AACGGCAGAGCACATGTAGTAATTTGTTAACACGAAACGGCAGCCTTCCA
ACTTTTGCACTGAAACACGGGGATTTACCCTTTGGGGGATTAAAAATGTT
GATACGCAAAGCCAAAGGCTAATGAATAGGATTCAGTGGCTTGCTGGCAA
TATTTAAATATTTCTGTCCGGGAGCGCAAACTCTTCTGCAGTTATTTAGA
AAAGTAGGGAGTTAAACCAAACTATAATACTCTCCCGTGCGTCTCCACTT
TCCCCAGCCCGAAAAGGAGCTACCAGCGCTGTACTGCCCCTTTAAGACCT
GCTGGGTCTCGGGCTCTACAAAAGGGAGTGACCTCTGGGCTTTGACAGCT
CTCCTAATAACTACCACGGCGCAGGCCCCGCCCACCTAGCTACCAGCAGC
GCCGATTGGCCGGCGGGCCGGTATCCCGCGCTGTGATTGGCCCGTCGCTT
CCCCTGAG[C/A]GAACCTTTAGAACTCTGAGACAATATTCTGTTACATT
GTAGCAAAATGGCGACTGTCATTCACAACCCCCTGAAAGCGTAAGTAAG
ACTAA

Following is a LOC145197 genomic nucleotide sequence (SEQ ID NO: 7).

| >14: 99854151-99949200 | |
|---|---|
| 1 | tccgtgttgc tgcgtgagta caatctctct ctctctctct cttttttttt tttttttttt |
| 61 | ttttttaaaa acagggtttc actctgtccc caagctggag tgcagtggtg caatctaggc |
| 121 | tcactgcagc ctcgacctcc acctccccca gctcaggtga ttcccccacc ttaatcttct |
| 181 | gactagctgg gaccgaccac aggcatgcac caccacgctc agctMatttt tgtattgttt |
| 241 | ggtagagata agttctcgct atgtcaccca gcctggtctt gaactcccgg gctcaagcac |
| 301 | acctccatcc ttggcctttc aaaggtctgg gattacaggc atgagccact gcatctgcca |
| 361 | cagtccccag tctgaatgcc tccctctggt gggctggagc tctgctccga gatggcatcg |
| 421 | agtcatcctt tctgcagagg ttgacgacgg ggcacccgcg tggagcactg tgggtcactg |
| 481 | tggcagaggc caggcagggc cagcRgggct ctggcacttt gcgcagccac cgggaagtat |
| 541 | ctggcatcac ccccactcac attccacgag gcatagcaag cctagtagaa taaacgtctc |
| 601 | cttgcaaaga tgactttcat ctcccgtttc attccatttt cctaacggag agaaagtaga |
| 661 | gaaaggcaat cccactgtgt gtccaaagag agccagacat atttggtggc cagtcctgaa |
| 721 | gactggcacg ttggacagca gctgtcctcg agtgaggaat gggacatgca tctagtcaac |
| 781 | tacaccgcct gtgtcctttc cactacgccg cagaaactgt gcagccgcac tgctgttttt |
| 841 | aattacaaag tttaagtcgt atgtatttta acactctttc aaaagttaaa agactgaggc |
| 901 | ataaaacctc agtatctctt aatctatgcc ttgttttaat gaatgtctat tgagtcctct |
| 961 | cagagagaag ttgctggaga atatacagta gcaccccatt atctgtggga tacgttccaa |
| 1021 | gacccctagt ggatgcctga aaccatggat agtcctgaac cctatatata aatatttgca |
| 1081 | catatgtcta tatctacaca catgcatata catacatgta tatacaccat gttttttttt |
| 1141 | tttcagtctg atcaccgaga cagctactaa gtgactaatg gatgggttgg atgagtggcg |
| 1201 | tctacagagt gaatatgttg gacaaaggga tgattcgcac ccggggtgag atggagcaag |
| 1261 | atgcagcaag atggcacaag atttcatcat gctactcaga ctggcatgca atgtaaacct |
| 1321 | tatgaactac ttatttctga aatgttttca tttaatattt tcagacctcg gataaccaca |
| 1381 | ggtgactgaa accacagaaa gcaaagccat aggcaagagg gggctactgc acggggcact |
| 1441 | gggtgacatc tgaaggtcag atcacacaca gactttagtg tgagtgtgtc ccaaatggtg |
| 1501 | cagggagccg acttatacga aaaaaattac tcattactta tctgtcattc aaatttaact |
| 1561 | agcatgctgg ggggagggtg tggtctgttg gtttgttttt gctaaatctg gcaaccctac |
| 1621 | ctcaaagggg gaggggctca ttctaccaaa caatgcatca aagcatgggt tttgtaaaat |
| 1681 | ttaaattata ggaaaacgtt taaggcaaaa ggtgctctaa tctctgtaag ggaggcagag |
| 1741 | ctcattccct aataacactg catgcacact cgaaggaaga tgtagtgaga taatttctgt |
| 1801 | gttcatccca gctccttgcc caacatgcac ccccaaagaa ctctggttgg ttggatctac |
| 1861 | tgcacctgct tctgaagaaa tctccaattt tggtggacac cctccctcta cattacccgc |
| 1921 | ctgacctttc agaacccagc gggccgggtc tcagcacatt acctgggata aagaagacct |
| 1981 | tgatatgtgt tcattaagca aaactgaatt ggaaggaaga aaacagagag ggagggaagg |
| 2041 | aaaaaaggaa ggaagggagg gagggaagga aggggaaggg agggagagag agagagaggg |
| 2101 | gcatggagag aaggaagaaa agaaggaagt aggaagggag gaaagaaagg gggaaagagg |
| 2161 | gcagaagaga agaaaggaag ggagggaggg agaaaggaaa gaaggaagga aggaaagaaa |
| 2221 | gaaggaagaa gagagggagg gagggaggaa aagagaggga aggaggaaag ggagggagag |
| 2281 | aggagaggag ggagggagga aaggaaggaa ggaaagtgat agtatgggat cccattttga |
| 2341 | ctggacatct ctgaaaatgc ccctgtatgc tcaccaaggc cctcaagaag gcactcaaca |
| 2401 | aatgctaatc aatgaagcct gttccctttc tcagagccac catcagcccc tacagcacct |
| 2461 | gcatgggaat caggacaggt gtccccttca ggtaagcaca gctcccagat gcagaggtca |
| 2521 | gtgagcggac accacctgtg gcttgtgcca gttggaacac ggcccccttt cccctggaag |
| 2581 | aagcaccgcc aggtcccaca gcctggccag cagagtagga ggctgcgccc cagcccttgc |
| 2641 | ctgcactctt cacatccgct ccctgctcct gatggaaagg ctaaagcatt gggaagtgct |
| 2701 | ctcagagctg gactttcacc ccttgtagaa aatgtcttct gcccagatta cccacaccca |
| 2761 | ccaaagagag gcagaattga aagaaagaga ggaaatctcc catcagcgcg tgcaggcaca |
| 2821 | cccacgcaca ccccagtgat cgccaggtgt gaactgtatg taaggtaaac aattccactg |
| 2881 | gagtggtgag tgaatggagc tcatccaatt cgatattttc tagatggagc gaatggtgtc |
| 2941 | tacattctcc tcacttgttt ttactttttt agccgtctac ttggtgtttt tggtgctgct |
| 3001 | taaaaatgat ttaaagaaac aaaaaaggca ttagggggtt gcacataatg aaatgaggac |
| 3061 | gactgcaacc aaggctgaca cttattcttt agttaattca attgtgccta attatcttgt |
| 3121 | tgctttgcaa tccagcctct cccccaacca gcaagagcct ccggaacatt ccttataatt |
| 3181 | gcagagcagc ccgagagctg agatgagtaa tcctcagcgt tctcattctc tgtcctgtcc |
| 3241 | ccgaatgcct ctcaggatgt ggaggagaaa acttcggaat ccagatcttt ctctgagctt |
| 3301 | tgtgaggagt ctctgcctgc gtttccctca ggcgtcatcg tgaatggccg gcagaccctg |
| 3361 | tgcacgtaga atttcctgtc tggctgtagg ggaaaccact ctgacagcca ggcagagatg |
| 3421 | aaataaccca actgaaaatt aattaactcc aaaacctccg cagaaaataa aacacaccaa |
| 3481 | gacatatatg ggccgcgagc aactttcagc tttgggaagg gatgggatgt gattattcaa |
| 3541 | tttttctagt ccttgactcc aggttcttac acattttctt ctgtgcctat gctgcgttct |
| 3601 | gtaattagaa atttttccaa cactctaaaa cttcaaaatt ataccctaaa gtcaagtttt |
| 3661 | caacaaaagc aggacttctt tggcttacct tcactgtaat ttggtttggg aaagtgtggt |
| 3721 | tttcagccat agcaaagaaa atggtcttca gaactcgaac tgtctttagt gaaatgttat |
| 3781 | ttttcaccaa atatgtgttc ccacaaagtg ttttctagca gctgggagag gtagccagct |
| 3841 | gagctctgtg ttgaaacgca tgccctgata cccccccgtg ccccatcctg cagcgaattc |
| 3901 | ttgggaccca tgcttcttag gacctttggt cctcaagcta tttagagatc catggaaact |
| 3961 | gcccaatgaa caaggacagg aaacagggaa aggctgccgc agcggattct tcttcttctc |
| 4021 | cttccccttc cccttctcct tctctttcct cttcctcttc cctcctccct cttccctctt |
| 4081 | ccctcttctt cttcgtgaat gagcgcactg atgaatagca gaggatgaga accctgcagg |
| 4141 | ccaggcacag gcagcatcaa agaaacagag ggtggagcac aggtgcccag cccaggagat |
| 4201 | ttaaaatcag atcagggacg ggcgcgatgg ctcacgcctg taatcccggc actttgggag |
| 4261 | gctgaggtgg gcggatcatg aggtcaggag atcgagacca tcctggctaa cacggtgaaa |
| 4321 | acccatctct actaaaaata caaaaaaaaa aaaaaaaaat cagccgggca tggtggcggg |
| 4381 | cacctgtagt cccagctact tgggaggctg aggcaggaga atggcgtgaa cccgagagac |
| 4441 | agagcttgca gtgagccgag attgtgccac tgcactccag cctgggcgac agagcgagac |
| 4501 | tctgtcaaaa aaataaaaaa taaaaataaa aataaaatca tcagatcagg cagaccaaaa |
| 4561 | ggccaagtca ggatattttg aagttgtacc ccctcctcgg acttcaccta atgctcccaa |
| 4621 | tgtacacaga atgttctgtc cctggatgga aggtgaccca aaatagagaa gaatcatgga |
| 4681 | aagacgaagg cggcagcctt tttgccgccc cacaacacct gacacccgag tgatgggtgt |
| 4741 | ggccctcaag ccaggaggcc tgcgtttggg tcctggctct gccgcttaat ggctgtgtga |
| 4801 | tgctgagcaa ttacttagcc tctccatgcc tcagtttcct cccttgtaaa tggatctaat |
| 4861 | gaagacacct acttccctta ctcttcgtcg tgggaaccaa gtgaactaat gcatgcagca |
| 4921 | tgctgaaccc tgcagccgct gtggcgaaaa cactcattga gatgcaagtc tccacgcaac |
| 4981 | agcaggtaaa gccccattgg aagtcatcgt ttataatctc aattatttcc ttgacctaga |
| 5041 | attgttcaat attaacagaa atgagatcag aattctttca gccaggcatg gtgccccaag |
| 5101 | agtgacagtc ctgaagccac tgctatttat ggagcccttt ccacagaaca cttccatgaa |
| 5161 | gcacggttaY cattcccatt caYggaagag gaaactgagg cacaatgctg tcacgtaatt |
| 5221 | tgcctacaca gccagagagc aactgagcca agattcaagc cctccgtctg tccacggcct |
| 5281 | gccctctcag ccactgtgca atgctgactg cagaccagcc acaactgttc tgccagggct |
| 5341 | ctgagtaacc ggaacatggt gatttaatca ggaggagata tttgcagaga tgttgaaatg |
| 5401 | ggggacatcc ataagtgaaa gcagtgattt gcaaaaataa tctggctgct gctttcccaa |
| 5461 | gcttcctcct atggaagcct cccctgtaac accgagtggc acaccttctc tgccctggcc |
| 5521 | ggagccgagg gcacagtccc tgacagagct cagcacccac ctccaagaca cacagggaga |
| 5581 | cagtcttggg gtcagcgaat ctgcacattc cgccggcact cgcagggcca gacaagctgc |
| 5641 | tctggcccaa tcttctcatt ctgtggtcga cacactgcgg cccggaggga gcagagggtg |
| 5701 | gaatttccca gggtcaccct ttggaaggat gccggagcca gaccctggcc tccccgaagg |
| 5761 | aaaataaagc agtggccctg agccggggcc acagagctcc atgtggccgg agcagggctg |
| 5821 | cggtgggaag gggaaggatg gagactgggg caccctcatc accggttaag gggctctcct |
| 5881 | gccaggcgca cgagacagaa cattcatgat gctctgagac agaggaaaga aggtacggac |
| 5941 | agcagggaag actctctgct cagccatctt tccaacatat tgtatttaaa ctatcaaatg |
| 6001 | ctttagccaa tctgcaaaaa ataaattctt tgcacagggg ggtgaaatct ggctctctcc |
| 6061 | tcagcagaag gcccttctcc acggctcccc ttggctccca cagaaaacac agggcagctt |
| 6121 | ctgcagcttc tccacaccat atatctagaa tttacataat cacaggcaaa taaaacgata |
| 6181 | aaagcacact ctttcataac aggcacattg ttactattat tattcttaag gcaaattagc |
| 6241 | ttctgattat agagattagt caaaaccatg attctgttga ttgtgaggta gaactgattt |
| 6301 | tcttttcttt cttttttttt ttttttcaac aaggagaaga gctgttgttt cgatgtcaga |
| 6361 | aaattacata aaatggtact cagagagaaa taaagtttct gaaagtcatt taattatcag |
| 6421 | ttctcttgga agcgtttagg cattccagcc tttcggttgt tgtctgtcta gccaaacatt |
| 6481 | ttaccctcaa gtttattaac tgtttgattt tattttattt aacctctttc ttttaaaagt |
| 6541 | ttctccaatt taaaatacca atattttaga tatttgtatg tgtttattgg gctttcttca |
| 6601 | tcactgaaat aattcatacc tcttttgaac caaagagatg cttgttcttt caccagcatt |
| 6661 | tttgatcaaa gacaaccagg aaggaagcat ctcctccctg ggcaaaatgg aggatccaaa |
| 6721 | accattaaga gtctgctgca agatgtcacc actcagactc agagagcttc tggctcctca |
| 6781 | gggcagcgcc aggcaccaga ggctccgtga aagcccttgg caggagagca gcactcacRt |
| 6841 | tgctcctccc tgccccctac ctgttttaat cattagtcct cagttaggtc cagtgtctga |
| 6901 | gccctgcctc cagagttgag tcccgcctcc tacctcacct gaactaactc tgaggttcca |
| 6961 | acctgggggt gattttgccc tctaggggac atctcacaat gtgtggagat ggtggRggtt |
| 7021 | atcagaatag tggggaggtg gtgggaagag gcagcgtttg agccacaggg cagtcccacc |
| 7081 | acaaagaatg atctggcccc caatgtcagt agggtccctg ttgagaaacc ctggtctaat |
| 7141 | tgatcagaaa tggtgggtga actttgaaca gataacacca gggagtcgtg cagctctagg |
| 7201 | ggaatcctct cggaccctgg agtgcccgat agaagcRgcg tgcacatggg gactctgccc |
| 7261 | agatccgagt ccttttgtcc atcatgatgt cccagattcc tgctgtcatt ttgcatagaa |
| 7321 | ttagactgga acatgcaaaa atatatacaa ttgttgatac caagatgtct gaatttagga |
| 7381 | agaattccca aacagaaaga tccctccccc aaaatgtatg catataaact tttaaggatg |
| 7441 | cagataaaat tgccagatgt ctttcaatct gattacctca catgctggcc tgtatcccag |
| 7501 | atgtgagatc cagagaaacg tgatggaatg tgtcactgtc ttcttttcac atcctctccc |
| 7561 | agatttcatt aatctgctga taggtactca ttaaaatact agtattcgcc ctgggacaca |
| 7621 | gtttcatgag attacttcac gaccgttaca ggacatatta cagtaaatga attttccttc |
| 7681 | ttttagcatg gtggattaaa aaaatacata attttgtgcc ccagtcctca aagaattaaa |
| 7741 | catttcagca tatcttacga caggatattg gcaacgtttt ctgtaatggg tcagatagta |
| 7801 | aatattttgg gctttgcagg tcacgtggtc tctgtcgcaa ctactcaaca gtgctatggc |
| 7861 | aggacagaag cagccataga tgatgtgtaa atgaacaagc ttggctgcat tccaataaaa |
| 7921 | ctttatttac aaaaacaggc tgtgggaagg acttggcccg caaccctggt ttgcctacct |
| 7981 | ctgtcttaca acatagattt cctgaaaata atgcatctag gcagaaagaa taggggaaag |
| 8041 | ttcagagtaa gacgttgtaa aatatcggca agagttctca tcgtcttaca agaagtagcc |
| 8101 | tgtgtacaac ttaatatcaa tccaaggagg accaagaggc atgccgaaat gagatttaaa |
| 8161 | ggttaaacag aaatctggag ggaaaggaaR aattgcaacc acactggcca atgatctagt |
| 8221 | gttgttactc tcatttattt gctaagtttc cctttcagtt taaagcacgg catttaattc |
| 8281 | agacattgtc cttcactccc catgagccaa tgaaagagga gatgcgtggg ctgtggaggg |
| 8341 | gctgagatgc ttgtggRcac ctggggcagc ggctgtgggc tgtggtctcc cgggctgtgc |
| 8401 | aggtcaggag gtcaggaggt ctctggagag aggcatcatc atggagctgg gcctgctgga |
| 8461 | gaaaggagtt ggccacggac accccaaaca ccccgatttg atcatgacac tttctgtgca |
| 8521 | tggaacccca catgtacccc ttaaaatttt accctgtaaa atattatgta tcaataSaag |
| 8581 | agagcaccaa agcaatcatc ctactagcca ccatatgcca tcctagcttc tccacagtaa |
| 8641 | cacgtcattt actcctcaga gcggctcagc gaggtagggc ctgctgatgg tccctttcac |
| 8701 | aggtgagaac acagaggccc tgggaagctg aggggtttgc tagagatcag gaagccagga |
| 8761 | ctcgggcttg ggtgctgggc tctgggcctg tgtgtctgac ttctccgagg gggtgctgcc |
| 8821 | ttctcctttc ctctctttct ccctaacccc cccacactca gccactaagt atttttagaa |
| 8881 | ttattactat ttaggtatca tctataaaac atgacattta ccattttaac catgttttca |
| 8941 | tgtatactcc agtggcatca agtacattca caccattgtg caaccatccc caccacccat |
| 9001 | ctccagagcc cccattcaat agaaaccccc ccttctcccc tcccccagct cctggccacc |
| 9061 | accatcctgc tttctgtcta tgaatttgac tgctcagggt gtctagagtg gagtcctgca |
| 9121 | gaatgagccc ttttgggtag aggtgatttc attcagcaca atggcctcaa ggttcactgg |
| 9181 | tactgtagcg cgtgtcaggg tttccttcca gtttaaagta gaatcctctg gttgtttgtc |
| 9241 | cattactcca tggacacctg ggtggctgcc acctgtgccc tggtctctaa gagctgctgg |
| 9301 | cactctcgct cctccctggt ccctttcctt gctcacctgc cctcccctgg ctcggctcct |
| 9361 | cctcccaggc ctccagcctg ccttcccggc accccacgcc tgctgcctcg ggaatcttgc |
| 9421 | ccaaactcca atctgcttgg gcctctctta ctggtaaacc cacaaggatc cccaccttca |
| 9481 | gcaaaaaaga gttccaaagg ctccatgtag gagcccaggc ccctcactga ctgccctggc |
| 9541 | cagcctctcc agtgttgccc caacggagcc ctgccctatg tacctaccct tgttttcctg |
| 9601 | aaaaccacac tctttcctgc ctggaggaca cccttctcac cctggagaga ttacccttct |
| 9661 | agaaacaaac caaacacacc ctctgcagag gctctgccag ccgccttctc caggcaaggc |
| 9721 | aggctgcctg cccccacgct cacggcagct tcctggcata cttgctgcct cggtttgtgc |
| 9781 | aaaagcaaac ctgcaagtcg catacttagg ggatggcccg ggaagcccat aaggatgtgg |
| 9841 | gagaacaaga cagggagggc aagaaggaca atcacagact catcctggag cacattcaac |
| 9901 | tgtgggacac aggaactcat ccctgagact gcctggaatg tagctcggaa ctgtcccaac |
| 9961 | agctcctaac cttccgaggt tgatggtccc tcctggttaa ctccgtggcc agcaagaggt |
| 10021 | cccaggaggc catcactgca cacagcaacc atccaccatg cctgcatgca ggtggcctct |
| 10081 | gggtggaaca gagggatgtg ggcaaaaatc tgggatgacc gcaagggcgc caggcacaca |
| 10141 | gtgggtttgc gactgtggta tctgcctctc ccacgggagg cagaccccat caccaatttc |
| 10201 | tctctgtacc cccagcccat ggcaggttgg agacatctga aaaaggtagc aaatgagtcc |
| 10261 | atgagtggct gtgtgggaat gagatggact gggtcttggg agagaggagg caggagtgga |
| 10321 | tgttggaaag acaggctgct gagaggagaa cacagtggca gggcaggtga gcagcggcca |
| 10381 | ggggctccct cacacacttc accccagccc ctgcgccagc ctcatctgcc tgtctcccac |
| 10441 | ctgacccttt tgccgttcac ctccgtcctc tctctgctgg gttctcaagg caatcgcacc |
| 10501 | ttgcctagca actgttgcta ccgaaagcca acgtccctgg tattccaaca ataaagcaca |
| 10561 | gcgctccgac tacaactccc agatattatt gaaaagaaag gcaggtttat ttcaaggcca |
| 10621 | ggaggggaga ggatctgcgt gttggaggct ggagcagcag cgcattcgat ggccggcctg |
| 10681 | acagcatggc gagcagcttg tgctggtgcg tcctccagaa aaagaatgcg cgctctccag |
| 10741 | gaggtgatgg tccctgttca taaactccct gttgctccct gttcccattc agctgggtca |
| 10801 | acatgtgcct gtaaattcct cccgaaaggg aaatcataac agcatcagca gccccagaaa |
| 10861 | tgacaaacaa ctcaagtatg gaaagccagc tgccaagtat tcaacagtac tcatgttgca |
| 10921 | ccagaaacaa aaaaaatcag ataagaaaaa taaacccagt tgtgtgcata gttccttaaa |
| 10981 | tatgtatccc attttgtggt tccttccagt cttcggtgta atgaatatat ccattggtta |
| 11041 | ggggtcacgg aatggcaggt cctaccaggc tgccttggat tggcgaactg tgatgttctg |
| 11101 | ctttctccct tctctgaagc atggagcggt gcgagacgtt cagaacagac agccactgac |
| 11161 | attaattttc ctgctggcct ccttgctaaa ttgttttatg acttgtttgc tgtgtcttgg |
| 11221 | ttgccctatt gtgttgagtt gatttggcag tggttattta aaacatgctt tgtcatcctc |
| 11281 | aggatgagtg gaggatcccg ggggtcactt ttgcacaaca aatggtcatt gagctgaaga |
| 11341 | actgtcagag tcaccacctc cacagtcaag tggcaatgga atccgaagac agggtcagct |
| 11401 | cggtacacca cgagccgaga tccacgggac ggacggtgtg caggcaccaa agcgtggaca |
| 11461 | ggccatgctt tgcttaacag catggggctg gccacggcac cagggcctct gccagcctca |
| 11521 | cgaggcccct cctggcgtgg ccataaaagc cacagctgca aacgctgaca cggcaagaaa |
| 11581 | agaatatggt gagaaaccag cttttgcctt tgcttctcag actcctcgga agatccgaag |
| 11641 | ggcactttcc catccccagg aatgagaggc cagattcctc catggtccag tcaggaaatg |
| 11701 | taatcaagca tcaacgcgga gccaggatct gtgcctcaaa cttgaaacgt ggccctatct |
| 11761 | tcaagggtgc tcacactcag atgacttaaa ccaaaaatga cacggtgtgg cctgcacttg |
| 11821 | cccatgccat agtcaggaaa gcctagtcaa ggacaacgca agcccagagg aagagggtct |
| 11881 | aaacccagcc gagaacagcc acccagagca gaggccctcc cctgagagaa agccacctgc |
| 11941 | tctcagccat ccctcttctt tccctacgag ctccaggaga cagaaaccat gatgcttagg |
| 12001 | tagacatcaa tctgttatgg caggcaccag aatgtttctt ctcctgtgtc ccatcaggag |
| 12061 | tcaaaactga tctcagcctg ggcaatgtag caagacctca tctctacaaa agataaaaag |
| 12121 | gttagccggg tgtggtgggg tgcgcctgta gtcccagcta ctctgggggc tggggtggaa |
| 12181 | ggatcgcttg agcctgggag gttgagactg cagtgagccg aaatcatacc actgcactcc |
| 12241 | agcctgagtg acagagcaag cccctgtctc aaaaaaaaga agaagaaaag tgatatctct |
| 12301 | gctactgagg acaaacatca tgcacccttt cttcttaaag aggagcaaat ttcccacttc |
| 12361 | caagtcctgt tagtgaaagt cccggaggag ccccagcctg agccatagca ggagagtcgc |
| 12421 | agagagatga aagggtctgt ccccagccca ctgtggcctc cagaggtagc atcttctgct |
| 12481 | cagcagcaga acaaacattt ccatggagca gctaagttga aaagccccca ggaggcaagg |
| 12541 | gtaggagtta caagaaacca gaaagtctac ggcaaggtca gggtgactcg ggtggggttt |
| 12601 | ctcttgcttc ccctttcaat agcattcatg aagtggctga aagtcttcat cttgttatta |
| 12661 | gctttgcttt ctggcctcgt ttatctctaa ttgttttcct aagccttgaa gaggaaattc |
| 12721 | tgttttcttt ctggtcctgc ttacatttaa atccacttaa gtttctgtct gtggctttct |
| 12781 | tctcaaaagg gccaccggct ccctggggga caagcatcct ccttataaag ttgaagggaa |
| 12841 | ctgggaagat cttgaaagac ttctctattg ttcatgcaga caagttgtgg gaatttattt |
| 12901 | ctgtgggtct gtttcctctt tgttcagggg gccccattct tctccaatta tctgcctata |
| 12961 | ccagagagta ttttcagaat ctgtgcagga tgttttagac tgaataagga caagcattga |
| 13021 | gggaggttga tattttgcta ataataacag tggaaaaagg taccatttct gaagcaccta |
| 13081 | ctgtgtgctg gaaccttctg tgtgcatcac attttaaccc tctcaactct ccaggaatgt |
| 13141 | attatgacac ctactctata gattcaaaaa ctgaagggca gagatgtaaa gaacttgcca |
| 13201 | gagttaccag cagtgagaga accaggggtc aaacccaaga ttgacccaag ctcctgctga |
| 13261 | ccccacgatt tcacgtggcg cccagtgaaa ctgtaagcaa agcaccactc cacaaatgcc |
| 13321 | tcactcatct ccacacccac ctgacacagt cattggattt taggacacgc atccttcttt |
| 13381 | caaagttcag gaatgttcac aagcaagtca aaattcattt tgaaagctcc tttgacttga |
| 13441 | ccgaagacgc cccaagatgt caccaaaagc caaggatggg aagccctgtt ctatcaaggt |
| 13501 | gctgaaaccc agtcctgggg agtgtttgac ttgggaagta ggctgccctc cttctgctca |
| 13561 | ccgtgggcag ggaccagaga gagggtctgg gattcaatac gagcctgatg ggggttctgc |
| 13621 | catggggtgt tggacggggg cgttcgacaa accagtgcag gctcaaaccc tgctcctctt |
| 13681 | gcccccagcc ccaagagtca ccgagaaaac aaacacagta agtctttctt aaacctccca |
| 13741 | ccaagcaagg gtgcaaagga agaccatggg cctcagcgtg aatgcccacg caccacagct |
| 13801 | cttggcatgg gtccccctgc ctgccacatg ttaccaccac tcgctttgaa gaacagcgtg |
| 13861 | ttcatcagca ctcacgaggc ttaacagcca cagtttaacc tagtaatgcc cagcccccac |
| 13921 | agcaggctag tcatcatggt cagtgagaaa cagccagact tttcacgcag atgggcattt |
| 13981 | tcggggccag tctctgcact ctccaagcag gagctggttg gagtctctgc aggctcaggc |
| 14041 | ccaagacggg tggccttccc agcagcttcg ccatatcagc tgccacgtgc tataccgatg |
| 14101 | tccttcgagg ctcatcctag gacttcatca aaaccgtttt tgcagatgcc aggactgtaa |
| 14161 | tattcaccac ttcccccaac cagcacatga ccttgatgga gctggaatca gccagatcca |
| 14221 | aacaaactgg gcgcaacgtc tgctctccag ctgcactgtg cttggggtgc actcaggggt |
| 14281 | ttgttcagct cccccgccaa agaattggag aagtcgtatt tcacatataa atatatttta |
| 14341 | cttctcagtt cactaacatc agcctctcat aaactcacta aatgaaaagc tgcaagaatc |
| 14401 | actttgttat ggatatgtaa aatgacatat tgcattggct aattatttct ctatggaatg |
| 14461 | aacagcccca tgactttcag gggccccaaa agagaacgct ctacaacaga agcacaaggt |
| 14521 | cattctttag tgggcaggta attgggaact ccatgctagg ctgtggctta ttagggattt |
| 14581 | gcggttcccc tcaatgcaca ttaactgaaa agctggacag tatctagagc aggcgtgcct |
| 14641 | tccatggcaa cagcatctcc gtcttcccag tgcatatggc tgtcaaagtc cttgtcaggt |
| 14701 | accagttcag aaacacataa cccatcctgg tatttttgta gtttcacaca tctagtggac |
| 14761 | tacttgtttg caataacatc atctggtgag cttttgagga tttgattctg tggcacttct |
| 14821 | tgaggaatac attttttaat atcctttaat ttttgcctaa ggacttggct gaatccaagt |
| 14881 | tagatccaca tgtaaccttt ttaaggaagg ctaaattgag acagaaaggc caagattttt |
| 14941 | ctccgagtag aaatggatat ttattactta agaaattgcc taaatgttgc tatcgaggct |
| 15001 | ttataaagac tcacttggtg tactggggaa aaaacacaca aagcaaaact agtccactga |
| 15061 | ttctgtacca attacaatgt ttgccttagg tgaattgcca atctatgagt catatttgtc |
| 15121 | tatatcactc tgcccagggt caagagaagt gtctcaaata agcaggatgt ggaaaagcgt |
| 15181 | ttgatcacgg tggcgatgcc gaggtgggga aaattccagt gataacctct gaggaaaata |
| 15241 | agccatttct tacaactaaa tcctttctgt catttctaaa atgaaaatta ggagcttatc |
| 15301 | aaaatgtaac atagcttaac tatcaagatc aattagagat ggaaataatt gggtagctag |
| 15361 | tgttctctac agaaagccag tgtgtaagga aacagtagtg ttatttgtag gaggcattta |
| 15421 | attatttcca agttgtggga caagtaataa acaagggtac atttctggct gtagaatttt |
| 15481 | gatctataaa tgtcctttga ggaatcagct tgttaggtgt attagtctgt tctcacactg |
| 15541 | ctgataaaga catacccgag attgagtaac ttataaagga aagacattaa atggactcac |
| 15601 | acttccacgt ggctggggag gcctcataat catggcagaa tgtgaatgag aagcaaagtc |
| 15661 | aggtgtcaca tggcggcagg caagagagtt tgtgcagggg aactcccctt tgcaaaaccg |
| 15721 | tcagaccttg tgagacttat tcactatcac gagaacagca caggaaagac ccgctcctca |
| 15781 | tgatttaatt acctcccacg tggtccctgc caccacatat gggaattatg ggagctacaa |
| 15841 | ttcaagatga gatttaggtg gggacacagc caaaccatat cattagcttt tcaaagattg |
| 15901 | tactttcaat tatgatcaag gttccatctt agaaaaagaa agcaaatgaa taaaataatg |
| 15961 | gtcttaaaac caaattccaa attttggttg cttcaaggYg taaggtctta caaaccatgt |
| 16021 | ttggaacgat ggtggatagt tctttgatga ctgacaaaga gttataaatt tcttctgtca |
| 16081 | ccttacagta gaaggagaat cagaactgat ttttttttcc ttcaaaaatt ccccgctggg |
| 16141 | gacaatgacc tcttccacag ctttagtgtg cacactcatg tacaatttga acaagcaagc |
| 16201 | catgccttct accccccagt ggcatccctg cattcctcaa cacaccaaag ggatctggag |
| 16261 | acaacaaagg ggtcagcaga tacaggtctt tctataactt agtgatacgt gtaccaagat |
| 16321 | agctgttttt tcaggggagg aggaggatat ttaagatatt ttaaacatct ttttaaaaaa |
| 16381 | ttggatggta ccaaaagata gagaattgta tcttccctat ccctagagac tcatgccttc |
| 16441 | ttccagaatg tcatgttacc tgattttctg tttgcaccgg gttgaatagt aaccccaaaa |
| 16501 | ttcatgtcta tctggaatct cagaatgtga ctttctctgg caatagggtc tttgcagatg |
| 16561 | caattagttc agatgaggtt atagtgggtt aggtcagacc ccaaatccaa tgactggagt |
| 16621 | ccccataagt agaagagata gacacaggga gacagaggca cagagggaag aaggtggcgt |
| 16681 | gttagtggag ccaaagacca gagctgtgaa gctgaggagc actgaggctt gctggcaatg |
| 16741 | ccggaaactg ggtcgaggcg tcctcaggga gcacagccct gctgatgcct tgacttcaga |
| 16801 | cttctggtct ccagaactgt gacaatatcc atttctgtcg tgttcagctc ccagtttgtg |
| 16861 | gtcatctgtc acggcagccc tcggaaccta atccgctgct gaaaacatgc cccttactga |
| 16921 | tgtgctgaac cctattccca tatttggaca aagaaatgga atcatgtggc atgaccaaaa |
| 16981 | ccatctgcat ctttagcagg ggcggcatcc taggctgcat gttaggatct cctggggttt |
| 17041 | ttttaatttt aaatcccact gcccaagctg caccccaacc caattaaatc agaaactctg |
| 17101 | gaggtagact cagacctggg tatttttaac cctccctggg tcattccagt gtgcagccaa |
| 17161 | gaccgtgaga accactgatc tctaggatgc agcagcgtgg ttttctcaca aaaccactgg |
| 17221 | cttcagagtt agtcaagttt tgggttaatt tcttgttttg ctacctccta gctgtgtcct |
| 17281 | tgggcaagtc atttaatgta tctgatattt actttctccc atctgcaatg agattgtgag |
| 17341 | gatccgtctt tttaagcatg taaaatatct gaaaatgcct ggctcaaagt agacagcaaa |
| 17401 | taaatgtcat tttcttttca ttctcccgac acatagaggg acaatttcaa aggacatgta |
| 17461 | agaatccagg ctgtagagtg aggctgtgag aatcaaatcc tgactccacc acctactagc |
| 17521 | tgtaaaacca tgggaacttc tcagtgccca gtttcctcac ctataaaatg gggcctcaga |
| 17581 | gagccattat gaggtttaca tgggataata aggtaatttg acccaggtaa agcacttaga |
| 17641 | atagtacctg gctcattgaa gcattcatta aatgttagct catattgtag atttacatta |
| 17701 | tagacattat ctgacaacac tYtgcacagc ccgccccccc ttggaaatgt gaacttcatg |
| 17761 | tggtacattt tagcattgct tctcttcgcc gtccagagca acatgatata cacaaaagag |
| 17821 | tcagtagaca gcagtccgga gacttgagct ctgagctccg ctgagatgcc actaagcttc |
| 17881 | aggatggaag acaacacttt tctctatgcc tcattccagg actcaaatta taaaagaaag |
| 17941 | ggtttactaa gctgatccct atggttcctt ctagcatata aaagttatga tcatctgaat |
| 18001 | attataaagc aatagatgaa gtttgggctg tgtttagtaa acaatctcta tgtttttatg |
| 18061 | gttaggattt tattctgagg ctagaattca cacaaaaacg tccagtatcg acagagaagg |
| 18121 | aatccaatcc atttcattgg aacaaagaac agccttagag atttttcaag aagtttcagg |
| 18181 | ccgagtcacc cagtctccca gtctaccgta taactgctgg ttgaagctgg agagaagaca |
| 18241 | ttagtggctc atgcacaaac tttaaagctt aatttatgtg agaacataaa ttgatacaac |
| 18301 | ctcactacta tgagattaaa taatatgtat caatagtcta aacaatacac agtaattatg |
| 18361 | tttctaggaa tctctcctga ggaaatacaa ttaaagtggg aaattagata atagggatta |
| 18421 | gtagtctaca atagtctaaa ctatacaaag taattacatt tctaagactc ctgaggaaac |
| 18481 | acaattaaag tatccttttt tcatgctttt gggaccagca attggagaat ctgcccagct |
| 18541 | caNtgaaaag ccagttaaat agggaaatct taaacaatgg tgcaaatata ccataaatac |
| 18601 | gttgttataa gttacaatat aaaaatatga attttctcac cagtcttgca ataaaggacc |
| 18661 | aaggctcttc tctgagtatc actcatgttc tgattggaat tatgtgccct ctttcttgca |
| 18721 | caaaacacgc ctatataatt catgctctta attttgagtt ttggtttctt taaagtggaa |
| 18781 | cacaaactta aatatttgga ggacaatgtg acagtagaca ctttctccat tttaccattt |
| 18841 | tattttctca aattgttcag tttcctggct cataatctag ttttatagca actgtattat |
| 18901 | tacttcactt tattgcatct ccccaaagca ttctacttag tttttatagt ttatatggaa |
| 18961 | gcaaccaccc tctttctttt ctcactcata tttcacaggt tagtattcca accaatgaca |
| 19021 | tcacaacaac tgtcataaac aggtttgaga acaaacacgg taattaactc ttggtaagcc |
| 19081 | catattccaa atagtgggag aaggaagact gaggcttccc gatgcaagcg gcatatttac |
| 19141 | cgaaggcata gaagacgtca gcgaatccag caaaacaatg cagggaaaca gtgattatga |
| 19201 | gggttccttc tgtagtcaaa tatgtcatca aaggtttatg tttaaagatg tttgaacatc |
| 19261 | tggaatagta actgatgctg gcagaaatat agaaactata taagtttaca atgagaaaat |
| 19321 | agctaagtaa cataaaacat atgatgaaat ggtatgtaga catcaaaaac tgcattttaa |
| 19381 | aaactatttt atgatataga agatgcccat attatgatct aatgtataat tcaataaaga |
| 19441 | tttaccaagc aatagaaaat tttgtaaatt ctgaaaaaaa agtctatttt ctatctttct |
| 19501 | tataaaaacc aaagtgggcc aggacttgga aaacgggact gatgtcatgt tgaactcctc |
| 19561 | gcttcctttg accttgatct atttggagtt taggcattgt ttttaagaag aaaacatgcc |
| 19621 | gcgcaggttc tctaataata aaatgcacat aacctcaaga aaaggtgggg tcatttttct |
| 19681 | tcttatatgg ccaagtaagc ttctcctaga ccttcctgca gataaccact atcaactttg |
| 19741 | gataaaaata taaaataagg cttctgttca ctagactgtg agtaaaaagc aggaagactc |
| 19801 | tagcagcaag ctagaggaaa aaaaatgcca agaggtgaaa ttcccatttt tatagctttt |
| 19861 | agcctgaggg catgtcaaag ttgtcaccat gtaagtgagt gaatctctaa aagaaaacgt |
| 19921 | tttgtttttc tgggctgaag acaacagagt tcagaacaac tagggtcact ggaggatgag |
| 19981 | ggagtaattc tggaaaagag acaagcacag aagaagcacc ccaaattttc taagtgaaat |
| 20041 | ctgaacctta tatgtgagag gtggattcaa agcagctaaa ctaagtgtaa aaaactgaac |
| 20101 | taaaattcaa actgctccag gggaaagagt tttcagtttg agtccaatca aattaattat |
| 20161 | ctgccctttg aaaaaatcag tgttcttccg agaaatataa cagaatccag atggcattca |
| 20221 | caatgtccag gatacgatcc aaaattacac aacagaggag agacaagaaa atgacctatc |
| 20281 | cacaagggaa aaataataga tgaagaccaa ttctaaaatg acccaaatgt aaaaattatt |
| 20341 | aaacgagaat ttaaaagtaa ggacagaagt gaaaatatgt tctccatgcc tgtaaagatg |
| 20401 | gaaagcatca tcagataaat agaaattata aaaaggaatc aaatgaaaat tctagagcat |
| 20461 | caaaatacat gtatgatatt ataatgttag acacatgtcc ttgtacattt gtccaaatcc |
| 20521 | atataatgta caacactgag aatgaacctt actgtaaact atggacttag gtgatagtga |
| 20581 | tgtatcaatg taggtttatc aattgtaatt aaagtaccac tgtggtggga gatgttgata |
| 20641 | acagtggaag gtatacatgg gggtggaggg tgggcaggga gtatatggaa aatctctcta |
| 20701 | ttttgctctg aacgtaaaac tactctaaaa agataagtat tttcttaaaa tgcaacttaa |
| 20761 | aaaattcttc atatggaccc aatagcagaa tggatatgat aaaagagtct gtgaacttga |
| 20821 | atataggcca agagaactta ctcaatctga aaaatgtaaa gaaaaaagat tggaaaaaat |
| 20881 | taacagagcc tcacaaaccc ctgtgacaat accaacaatc tactatcagt gtaaatggta |
| 20941 | gaatggaggg aaaagtgaga ataggaaaag agggaaacat tttgaaaaaa taaggaccaa |
| 21001 | aaagtttccc aaatttaatg aaagacataa atttacagat aagaaagtca gaaagcctcg |
| 21061 | agcagaaaac tacagagaaa accacaccca atcacccata gtcaaacagt tccaaagcaa |
| 21121 | agagaaagag aaaatcttga aagcaactgg agaaaaacaa aacataatgg acagaagaac |
| 21181 | accacatcaa atgacccagt ttatcattac agaaggccag aatatactag aacaatatat |
| 21241 | ttaaagttct aaatgaaaaa ctttaaaaac ctattaaccc agaatctgat accaaatgaa |
| 21301 | catatccttc aaggattaag acaaaataaa aacaagttca tataagagat agctgacaaa |
| 21361 | tcactgacag cagatctcta caggaagttc tttattctga agggaaatca tatcacatgg |
| 21421 | aaattcagat cttctgcaga aaaggcaatc tactggaaat ggtgaatata taaaatacct |
| 21481 | gggaagtata gaaaactttt cttcttgatt tgtttaaaaa tatgcacaac tggccaggct |
| 21541 | tggtggctca cgcctgtaat cccagcactt tgggaggcca aggtaggcag atcacttgag |
| 21601 | ctcaggagtt tgagaccagc ctgggtaaca tggtgaaaMc tcgtctctac aaaaaataca |
| 21661 | aaaatttgct gggcatggtg gcacaaacct gtagtcccag ttactcggga ggctgaggca |
| 21721 | ggaggatcgc ttgagcccag gaggcagagg ttgcagtgag ccaaaatcac accactgcac |
| 21781 | tccagcctgg gtgacagagt cagaccctgt ctcaaaaata aaataaaata aaataatata |
| 21841 | tatataaaat taaataaaat aaaacaaaat tttatattat ataaataata taaaattata |
| 21901 | ttttatatta tataaataat ataaaattat attttatatt atataaataa tataaaatta |
| 21961 | tattttatat tatataaata atataaaatt atattttata ttatataaat aatataaaat |
| 22021 | tatattttat attatataaa taatataaaa ttatatttta tattatataa ataatataaa |
| 22081 | attatatttt atattatata aataatataa aattatattt tatatataat aaaataaaat |
| 22141 | atatataaaa tagcttatat atataaagct atttaagtga aaattatagc attgtcgggt |
| 22201 | tgggtttaca ctgtaaatgt aatgtgtaca gtaactacag tatgaagtaa atggagataa |
| 22261 | atgaagcaat gtagctgcaa agtttccaca ttttacatgg tgagctacaa tattaacact |
| 22321 | aagtggactg tgataagtta aggaggcata ttgtaatctt tagagaaatc attaaaaata |
| 22381 | cagaaataaa tggcttaaaa gtcaatagat aaattaagct gaaattattt ttaaaaatca |
| 22441 | aataatttaa aaagaaggct gaagagaaga ctggaggaac aaaaaactac ataggacaaa |
| 22501 | aaagaaacaa acaaaaatgg tagacctcaa tccaaccata taaaaatact ttaaatgagt |
| 22561 | aaataattag aaggcagaac tagaatagaa ggaagaacta gaatagaaat agaattttaa |
| 22621 | aaggaaaaaa tcctggaccc atctataaac cgtctgtaag agaagcacat ttttaaatat |
| 22681 | aaagatacag ataggttgaa agtaaatgag tggaaaaaat ctacattatt atatggaagc |
| 22741 | ataagaaggc tggagtgaat atgttaatat cagataaaat aggcttcaag aattgcggcc |
| 22801 | ggatgtggtg gcttatgcct ttagtcccag cactttggga ggctgaggtg gatggatcac |
| 22861 | ctgagatcaa gagttcgaga ccagcctggc caatatggtg aaaccccgtc tctactaaaa |
| 22921 | atacaaaaat tagccaggca tggtggtggg tgcctgcaat cctagttact tgggaggctg |
| 22981 | aagcaggaga actgaggagg cggaggttgc ggtgagctga gatcatgcca ctgccatcca |
| 23041 | gcctggctga caagagcaaa gctccatctc aaaaaattaa aaaaaaaaaa aaaagaattg |
| 23101 | aatattttac agacaattac accccaaacc tggagaatat atttcttttc aaatgtacat |
| 23161 | ggtatattca tcaatacata aaacataaga caagtctcac tagaatcata tgaagtatgt |
| 23221 | tctctgacta caaacgaatt agaaatcaaa caacaataaa atatctagaa aatcccaaat |
| 23281 | acttaaaaac taaacaacac acttctaaat aatgtatggg tcaaagaata agtcacaaga |
| 23341 | gaaatgggga aatatttgaa cggaatgata ataaaaacac ataaaaattt gtggaattga |
| 23401 | cctagatctg tgcttaggaa gaaattaata gttttaaatg tttatatttg aaaagaagag |
| 23461 | agatctaaaa tcaagaatcc aaggttccaa ctaaaggcac tagaaaaaga ggagcaaagg |
| 23521 | aaactcacaa agttagtaga cagaagtaaa tactaaacat aacagcagga atcaatgcaa |
| 23581 | ttagaaaaca aaaagtacca aaaaatgtaa tcaaaagctt gtttttgaaa gataaatatg |
| 23641 | agttgtatac ctctagttat tctgatcaat aaaatagaaa gggcacaaat taacaatatt |
| 23701 | aagaatgaca tgggagacat ccctgcaggt gccatggata ttaaagatat agtaaaagac |
| 23761 | tatgctaact ttaaattaac acattggaca acttagatga aatggacaaa ttctttaaaa |
| 23821 | cagaaaatct caatactcat atatttatca agaaattagc ttctttatca aaaacatctt |
| 23881 | cacaaagaaa acttcagacc cagatagctt tattagttaa ttctatcaat acttaaggaa |
| 23941 | ataataacac caaccacata aaattcttcc taaaactaga ggaggaaacc tttcctaact |
| 24001 | tgttttatga ggccacagaa accctaaaat caaagcctaa caaagatgtt acaaaataaa |
| 24061 | agaaaattat aggccaatat cattcacgaa catagatgca aaatgcttaa caaaatatta |
| 24121 | gtaaacagaa tacaatatgt aaaaaagata gtatatcatg atgaagtggg gacatatccc |
| 24181 | agaaatttaa ggttaaatta acattcaaaa atcaatcagt gtaattcata ttattaacag |
| 24241 | acggaaagag acaaaccata tgatcatttc aacagatgca gaataaacat ttaacaaaac |
| 24301 | tccacattca tttatgagaa aaattagaaa taacgttata catagggcta tcatatttaa |
| 24361 | tgatgaaaga tttcaacttc tcctgttaag atacggaaaa aggcaaggat gcccacttcc |
| 24421 | accattctta ctcaacattg cactggaggt tctagccagt acaataagat gacaaaagta |
| 24481 | agtaaataaa caagtaaaca aagtcatcca gagtaaaaag gaagaagtaa actgtatcta |
| 24541 | tttttagata acataattgt ttacatagaa aaatccaata gaatcaacca aaccaccact |
| 24601 | agaactaatt catgaattta gcagtttggt agaatctatc cttctaccaa aggatagaag |
| 24661 | gtaaacatgc aaaaatcaaa tttatttaaa aaataaattt aaaaaataat tccacttata |
| 24721 | atactatcaa aaaacttaag atacataggg acMaatttaa caaaatatgt tcaagactag |
| 24781 | tactctgaaa attacaaaac attttggaag aaattataga aggtataaat aagtggagag |
| 24841 | atataccata ataatgggcc agaagactaa catgtcaatt ctctccaaat tgatctatag |
| 24901 | attcaatgtg atcccaattt taacctgcac ctgttttgtg ggaattaata cgtttattcc |
| 24961 | aaaaatctat atggaaatgc aatgaacctg cagctgcacc atacattatg gtggccaata |
| 25021 | actacatgtg gctacttaga tttctacgta ggttaatgca aattaaataa aattaaaaat |
| 25081 | taagttcctc caccacacta accacatttc aagggctaaa tactcacatg tgactagtga |
| 25141 | ttactcttct taaatggtgt agatgtgtaa catttttatc actgcagaaa gttctgttgg |
| 25201 | acagcactga cctcaaatag ccaaagcaac cttgaaaaaa accccaaaaa caaaaaacta |
| 25261 | gaggacttac atacactacc tgacttcaaa acatactaca aatctacagt ggtccagaca |
| 25321 | ttgtggaatc acatagacac aggagaaaat gttcaggact taagaatagg caaaggttta |
| 25381 | tttcacaagt cacatgaaac aaccataaaa gtagaggtag acttcatcaa aattagaatc |
| 25441 | ttctgctcat caatagacat tctaaggaaa atgaataggc aagccacagt ctgagagaaa |
| 25501 | atatttgtaa aacaagcatc tgacatagca ctagttccca ggaaacataa ggaacccctc |
| 25561 | caacccaaaa ataaaagcat aaataacccc tctcctcaaa aaactggagt gagcaaaggt |
| 25621 | tttaaaaaga aattttccaa aagaagatat gcaaatgttc aatatacaca acaaacagtt |
| 25681 | ctcaacatca ttagaaatca ggcatatgca aattaatcag gcatatgcaa attaaaacca |
| 25741 | caatgataat accactacac atctattcaa ataactaaaa ttaaaaacaa tgaaaacacc |
| 25801 | aagtgttggt agggtgcagg gcaactggaa ctctcataca ttgttggtga gaatgtaaaa |
| 25861 | tgatacagcc actttggaaa aataccttgc agtttcctaa aaaactaatc acacacctgt |
| 25921 | gacccagtaa gtccacaact agatttctac ttaaagaaaa taaaaacata cactcattca |
| 25981 | caaaaagact tatgcaaaag aaaatagcag atttattcat aatggcctga aattgtccct |
| 26041 | gtgttcttta acagaaaaac agacgaacaa ttttttttat tattatactt taaattctgg |
| 26101 | ggtacatgtt taatatattc ataaaacaga agaagattta gcaacaaaaa agaccaacta |
| 26161 | ttaatacatg gaacaagatg gatagatctc aaaaatatgc tgagtaaaag aagcaagacc |
| 26221 | aaaaaagggg catatactgt atggttccat ttatatgaaa ttttagaaaa ggcaaaacta |
| 26281 | agctatacRg ttagttagca gcgtcagaag aattgttgtc tctggggatc tgaggatggg |
| 26341 | aacaacttgg gaagtagcat gaggaaagtg tctaagtgat ggtaatgttc tacatctggc |
| 26401 | taggctttgg attgcagagt atgaacttgt caaaattcag tatattcatt tactactgtt |
| 26461 | gcataacaaa ttaccacaaa tgtagagact taaaacagca cacatttatt atctcatagt |
| 26521 | ttttgttagt cagaagtcca aacacaactt ccttgggtcc tcttctcggg atctcacaag |
| 26581 | cctgcaatca acatgataac caggctgtgt tctcacttga aggtttgact ggggaaaaat |
| 26641 | cttccaagtt cattcaggtt gtcagaagtc atttccttgc agctactagg ctgaaggcac |
| 26701 | tagcttttag ctgggtatca tctggagacc atacttaggt tctagagccc acagttccta |
| 26761 | gaaagtgccc tccatccttt gcaatccagg tttctcccag aaggccactt ttttcaccaa |
| 26821 | actcgcaaaa gtctctggtc ttgtctgcta gcaagatgga ggtgctgatg gacagtgccc |
| 26881 | ttagaccttc tagaggtcct ctggtttgat tgagaggatc tgggaggcat gccctcttaa |
| 26941 | aagatacctt tttgtgacta aatatgctca ccttttgatc tttcagattt tagcaaaggg |
| 27001 | ttgcaaagtc acaccctcta atttttctct agaccacact gtcttaattt gagaatctat |
| 27061 | tgccatctgg agaggcttaa aattttcaaa atcatccagt cctagttcct ttctgtttaM |
| 27121 | gaattctctt gatttatctt tctctcctct cacattttac tgtaaacagc cagaagagac |
| 27181 | tgggatgcct tcaacacttt tcttggaact ctcctttgct acataaccaa gttcatcacc |
| 27241 | tacaagttca actttctaca taactgcaga caacaatgtt gctaagcttt ctgtcactgt |
| 27301 | ttaacaaagg ttctcccttc tgtcagtttc ccattacgca ttcctcactt cctcctgagc |
| 27361 | cctctcctgc agtgtcctta aagtccaggc ttctattact atcagtcttt tcaggaagac |
| 27421 | ttaagctttc tctattctac tcccctgccc actgctgagt ttcaaaacca cccaacgttt |
| 27481 | tcagatattt attaacagcc acaccccact tccaggtatc aacacttgta ttagttttct |
| 27541 | attgctgtgt gacaaattct catagatgca tttatgatct catagcttct ttagagcagg |
| 27601 | agtccaggta cgacttagct gcttccctgc tcagaattcc acaagtcttc actctcctct |
| 27661 | gaaggctcca ctgggggaga atccatgctg gagcctactc ttacagttgt tttgagaatt |
| 27721 | atttccttgc agctgtatga cccaggaccc cagcattttg ctggctgttg gttggaggct |
| 27781 | aacctcaggt tctgggggca agacacagtt ccttgctaca tgggcttttc caatatggtc |
| 27841 | acttacttca tgaaggccac aaggagtctc tagctccagc ctaccagcaa aagggagtct |
| 27901 | tacaaaatgt taacataacc ataggagtga catcccagca cctctgccat atgcatctgc |
| 27961 | tactggttat gagcaagtta taagggatta cccaggatgt gagtatcatc aggggtcacc |
| 28021 | ttaatgtatg tccaccgtac tcagagaatg cacacttcag atttgggcat tttacataaa |
| 28081 | aaggaaaagg tagattggac tctagatatg catgctaaat tgtttaggga gaagtacacc |
| 28141 | gaaatgcatc agtaaaaaag atgaattgat ggaaggatag agggacagat agatgaataa |
| 28201 | atatataatg acacaagtgt aataaaatgt caatggaaga atctaggtgt gggtatctga |
| 28261 | gtatttgctg aaaatttagt tcaatatttc tgtatattta aaaattttat aacaaaatat |
| 28321 | tagaaaagta aatgtatact tattaaaaag agcacagaca taaatgagtc atctatttct |
| 28381 | ttatactttt cagcattttc tcactttact ccaataatca tgaattttgt gtataatcaa |
| 28441 | gagttggggg ccgggcacgg tggctcacac ctataatccc agcactttgg gaggccaagg |
| 28501 | tgggcggatc acgaggtcag gagatcgaga ccatcctggc taacacgatg aaaccctgtc |
| 28561 | tctactaaaa aaaaaaatac aaaaaattag ccgggcatgg tggcgggcgc ctgtagtgcc |
| 28621 | agctactcgg gaggctgagg caggagaatg gggcgaacct gggaggcaga gcttgcagtg |
| 28681 | agctgagatt gcgccagtgc actccagcct gggggacaga gcgagactct caaaaaNaaa |
| 28741 | aaaaaaagag ttgggaaaat gcatttagtt tataaagcag tcaatatatc aagccagcga |
| 28801 | tctctccagt ccattactga gtctttccct gctttcccat gaatcagtca ccaagagaac |
| 28861 | agaaagttac ccccacctcc aaacattctt gaggttttct actagccagc tgtggagtga |
| 28921 | ttattcctct gttcacctaa acctctgaaa ttgcacattt ttaggatgca ttatctcttc |
| 28981 | gacataacag ttgcataata gtaccacaag ttgcatacaa ttgctcttct atttatatat |
| 29041 | tataaaactg cctccttaat attttagatt ctcccctctg gttctaatat ttttaaattt |
| 29101 | ggtaaataaa ttgaaattca cgctaatcct agtgattcag attctaccat aaaaatcaat |
| 29161 | actatgtaaa actggagtgc ttaaggctaa atcaacaaca tatgtagtca tacctattag |
| 29221 | tgtcctgcgt ttgtaggtaa attaattagt attacttgga ccacatcata ccacagccaa |
| 29281 | taaaaataac agatgtttgg agaatgaagt aactggtgta tacaggccta gacattggct |
| 29341 | tttttgtttt tgctttttac aagagagaat ggttctgtcc agtcaagcac attattaagg |
| 29401 | cattcatatt taataagtgg gtcctaacat aggaaggaaa gttctaaggc tacttattgg |
| 29461 | cattaatata ttatctatgc tcccctcaaa attatttaat caataagtat cagtcacctc |
| 29521 | ctctgtgtta agtctattct agagaggata tagaaaagga gWgagagtaa atacacaaag |
| 29581 | tcattagcaa agaatacaat acagtatcta gctatgtgct aaaatcacta acagttcaat |
| 29641 | acagcatttg ttaggctaga tactaagaaa ttagcaacag aatacaatct tcctcctgcc |
| 29701 | ttaaagcact cctggttctc tgcagaacag agctgagatg taggacatca gtaaatcaga |
| 29761 | cctagaaaag tcaaggggtt tacagagatg gcaggaattc gtgggaaaga aagccaagat |
| 29821 | cttcaggatg ggttggcttt agacggcatc agtaaaacca aggaccagcc ttcagtatca |
| 29881 | tctttagttg tggggtaaaa caactgcaac cccaatgtta ctgcctgagg acttaacagt |
| 29941 | taacctggct gccactggga tgggcaataa gctcaaacca cagcatggag ttttgggggg |
| 30001 | accatgtctc tcgggacctg gccaatatga tgaggataaa ggatgaataa gtaaaccctc |
| 30061 | aatgatgatt ctctcaaYac tctcaaccag tgttctcagt gaaggttggg gggcatcgtg |
| 30121 | gaaagctagg gggaaacgat tttgctgatc acattatttg agaggtgctt tttgaatttt |
| 30181 | caactgtatt cacctaaaat aaatacatga taaactgcat atatttaagt gtactatatg |
| 30241 | cttatttaag tgttttttcc acacgcacac acagacacac acacacaaca aacctgcaag |
| 30301 | accatggcta cactgaagat gataaaaata ttcctcactc tccttcacaa aggtctcatg |
| 30361 | cccacttgtg atgcttgcat gcccctcccc tcttccatca tcctcaggca aacattgatc |
| 30421 | tgctgtcctt atagattact ttgcattttc cagaatgtca tctaagtgaa ctcatattgt |
| 30481 | atgtattcta tttgtctggc ttctttccct ctgcataatt atttcgagat tcatctatgt |
| 30541 | tgcagcacgt gttaatagtc tatttctctt tattggcgag tagtagtatt ccactgtctg |
| 30601 | gatataccac agttattcta ttcatctgtg aataggcatt tgagttgttt ccagatattg |
| 30661 | gcaattacaa ataaagctgc aatgagcatt tgtgtccagg tctttgtatg gatgtatgtc |
| 30721 | catacataaa tcatacacaa atcatttctc ttagatgaaa tggaatggct cgcactgttt |
| 30781 | tccaaagtgt cttactcatc tcacaagccc acctacagag tttcagatga gccacatcct |
| 30841 | tgccaacact tgctatggtc agtctttaat ttatccattt caactggttt aagtgctatc |
| 30901 | tcactatgtt tttaatttac agttccttaa taactaatga cactgagcat cttttcctgc |
| 30961 | ccttatttgt catYcatata tcttctctgg tgaagtgtct attcaaattg tttgcctaat |
| 31021 | tctttttttt ttttttactt taagttccag gatacatgtg cagaacatgc aggtttgtta |
| 31081 | catagataga catgtgccat gtggtttgct gcacccattg acccatcatc tgggttccct |
| 31141 | cccctcactc cccatccccc aacaggcccc ggtgtgtgat gttcccctcc ctgtgtccat |
| 31201 | gtgttcatat tgttcaacta cctcttatga gtgagaacat gctgtttggt tttctgttcc |
| 31261 | tgtgttagtt tgctgaggat gatggcttcc aacttcatcc atgtccctgc aaagaacata |
| 31321 | atctcattcc tttttatggc tgcatagtat tccatggtgt atatgtacca cattttcttt |
| 31381 | atccagtcta tcattgattg ggcatttggg ttggttccat gtctttgcta ttgtaaatag |
| 31441 | tgctgcaata aacataaatg tgcatgtgtc tttttttttt ttaagttgga gtctcgctct |
| 31501 | gtcacccagg ctggagtgca gtggcacgat cttggctcac tgcaYgctct gcctcctggg |
| 31561 | ttcatgccat tctcctgcct cagcctcccg tgtagctagg actacaggcg cctgccatca |
| 31621 | cacccggcta attttttgta tttttagtag agacggggtt tcattgtgtt agccaggatg |
| 31681 | gtctcgatct cctaacctcg tgatctgcct gcctcggcct cccaaaatgc tgggattaca |
| 31741 | ggcgtgagcc actgcaccca gcccacatgt gtctttatWg tagaatgatt tatacccctt |
| 31801 | tgggtttata cccagtaatg ggattcctgg gtcaaatggt atttctggtt atagatcctt |
| 31861 | gaggaatagc cacactgtct tccatgatgg ttgaactaat ttatattccc accaaccgtt |
| 31921 | ttaaaagcgt tcctatttct ccacagcttc accagcatct attgtttctt ggctttctaa |
| 31981 | taattaccat tctgaccgga gtgagatggt atctcattgc ggttttgatt tgcatttctc |
| 32041 | taatgatcag tgatgttgag ctttttttca tctgtttgtt agctgcataa atgtcttctt |
| 32101 | ttgagaagtg tctgtttata atctttgccc actttttgat gggttttttt ttcttgtaaa |
| 32161 | tttgtgtaag ttccttgtag attctggata tcagaccttt gtcagatggg tagattgcaa |
| 32221 | aaattttctt ccgttctgtt ggttgcctgt tcactctgat gctagtttct tttgctgtgc |
| 32281 | agaagctctt tagtttaatt aggtcccatt tgccaatttt cacatttttt gcaattgctt |
| 32341 | ttggcatttt catcatgaag tctttaccca tgcctatgtc ctgaatggta ttgcctaggt |
| 32401 | tttcttctag ggtttttatg gtttggggtt ttacatttaa gtctttaatc catcttgagt |
| 32461 | taatttttgt ataagatgta agaaagaggt ccagtttcag ttttctacat atggtttgcc |
| 32521 | taatttttaa ttaaattttt tatcattgag atttagagtt ctttatacat tctggataca |
| 32581 | agtctttatc agatacataa tttataaata ttttctccag ccttgtcatt ttcctaagaa |
| 32641 | tatcttttga agagcagaag tttttaattt tgataaagtt caatttatca atttgttatt |
| 32701 | ttatggatca tattatggtt gttctaacta agaaatattt gtctaaccca agtcataaag |
| 32761 | gttttcttct ataagttcta cagctttaga ttttatattt tggactaggg tccattttga |
| 32821 | attaatgttt tttatatgct tagatcaaaa gtcccagcac aaactgttga aaagaatatt |
| 32881 | cttcctccat tgccttgggg agttgtctgt ataagtgtgg gtttgtttct gaactttatt |
| 32941 | ctgttccact gatatatttg tatattttaa gccaatactg cacagggttg attaccatca |
| 33001 | gtctatgata ggtgttgaaa tcaagtaatg ctaatcattt ttaaagttgt tttggctatt |
| 33061 | ttaggtcctt tatagttcca aaggaatttt agaatcagtc tgtcgagtgc tacaaaaaaa |
| 33121 | aaaaaaaaaa aaatacttgc tcggattttg atgaggattg tgttaaatcc atatagcagc |
| 33181 | tttgggagaa ctgacatgtg aatagtaaga acgcttcaga tccatagaca tactgtatgt |
| 33241 | cttctccact tgacgtcttc tttgatttat ctcctcaatg ttttgtatgt ttcagtgcat |
| 33301 | acaacttgca catcttctgc caaatttatc cttacatatt ttatattttg gatgctgtta |
| 33361 | taaatgatat tcctgaattt caatttatga tcttttttat tcttaatata tagaaatgca |
| 33421 | attgatagct gtgttttgat tttgtataat gcaaccttgc taagctattt gttctggtag |
| 33481 | cttttattgt agattcatat aatttttcaa gacaaccaca tcatttgttc ataaggtcag |
| 33541 | ttttacttct tcttttccta tcaagttgtc tttcgtttct ttttcttgcc tgattgtctt |
| 33601 | ggctaggatc tctggtatga taatgaatag aaataggaag agaacatatc tgtgtcttgc |
| 33661 | tcttgacctt agagtaaaaa cattcagtct ttgacacaag ctaaagaatt tatgtagatg |
| 33721 | cccaatatca cactgaggaa atcccctcga ttcctagttt gctgagagtt gtttttagaa |
| 33781 | atggatattg gattttgcta aatatgtttt tgggtaaaga gaaggagggt gtctattgaa |
| 33841 | atgaccactt ggtttttctt tctcagtttg ttcatatagt gtgtatatgt gtatctacta |
| 33901 | tacacatatg tatagtgtat ctactataca atgtagattg attttccaat gtaaagtcaa |
| 33961 | cattgcattc ctgacaaacc acacgtggtc ataatgtgct atcgtttttg catattgttt |
| 34021 | cattcaattt gctaaaattt catttagcat ttttgtatcc aYgtttataa aggacacttt |
| 34081 | ctgtgattcc cttttcttac aatgtctttg tcttattttg gtattaggat agtactagcc |
| 34141 | tcacagagtg agttgggaaa ggttccttcc tcttcaattt ttaaaaaatg tttgtagaga |
| 34201 | acaagtatta tttctccctt agatgtttgg tagaattcac ctgtatagcc atttggcttg |
| 34261 | acatctcact gtgggaaagt ctttaacaac aaattccatt tctacagtag acatagtgtt |
| 34321 | attcaggtta tttaactttt aattggtaag cattgatgat ttgtatcttt caaggtattt |
| 34381 | gtccatttca tctaagttgc aaatttattg gcataaaatt tttcatatca ttcccttatt |
| 34441 | gtattataaa taactggaca agctatagtg atatcacctt tctcactttt tttttttttt |
| 34501 | tttttttttt tttttggaaa caggttctca ctctgtcacc caagctggag tgctgtagtg |
| 34561 | caatcatagc tcgctgcagg ctcaacctcc tagtctcaag caatcctcct gccttagccc |
| 34621 | cctgagtagc tgagagtata ggcacaggcc accacacctg gctaattctt tatttttaat |
| 34681 | ttttttgtag agactggatt ttgcagagac tgtgttgccc aggctggtct tggactcctg |
| 34741 | ggctttagca atcctccctc cgaagcctcc caaagtgctg ggatacaggc ttgagccact |
| 34801 | gctcccggcc ctttctcatt tttgatactg Kaatttgttt cttctttttt cctgatcaat |
| 34861 | ctgtatagag ttttatcaat tttattgatt tatttaaaga accagStttt gggttcattt |
| 34921 | gttttctcta ttgKctgKtt ttattttttc attgacagcc actctggtct tttttatttc |
| 34981 | ctttcttcta cttactttga gtttactttg cccttccttt ttatatttgt taatgtggaa |
| 35041 | agcgagatct ttcttttttc ttaatatagg catttcatgc aacaaaattc cgcttaagtg |
| 35101 | ttcctctagc agcatcctac aaattctgat Rtgtagtgtc ttcattcagc tgaacacgat |
| 35161 | cccaatggtg ggagctagaa caattggagc aacaaaacaa ataacacagt attggcttac |
| 35221 | agcccaaagc ataagataaa tatccatgag tctatcctga tagaaataga tgattgagta |
| 35281 | aataactaaa ggaggacaga agagctgagt ctcccatgca gaacaactcc aaatacttta |
| 35341 | tgtagattct ctgccagcta ggagacagag cctagctccc tgctcacgtg ggagctgtgc |
| 35401 | ctagagactt ccatccaaag atgatggcat tggaaagggg taatgagtga ctgtccagag |
| 35461 | aagaaggctg acaaacacta cgtccgtctg gtgatgaagg tcaacattaa Yggtgataag |
| 35521 | ccatgttgat ggtgggcacc cttgggtggg atgtgatgag aatggatttt atctctgtag |
| 35581 | tcttccttcc aaaacccgta agcccatata atcacaagaa aaacatcaga caaatccgat |
| 35641 | ctgtgggata ttccacaaac tacctgccca ggactcctca aaactgtcag ggtcctcaaa |
| 35701 | aataaggRaa gtcagagaaa ttgtcaaagc caagagaagc tgagagagac atgatgactg |
| 35761 | aacataacat ggtgtcctgg atcctaaaga gagaaaacta aaacaacagg aataaagtat |
| 35821 | ggactttagt ttaaataatg tgctcatatt ggtgcattaa ttgtgatgag tatgccatac |
| 35881 | tattgtgaga tgttaatctt aggggaaact gggtgggggt agatgggaac tctgtgctat |
| 35941 | ttttgaaaat tttctgtaaa tctaaaacta ttctaaaatg caagtttctt ttcttttttt |
| 36001 | tttttttttt tttttttgag gtggagtctt gttctgtcac ccaggctgga atgctgtggt |
| 36061 | acgatcttgg Stcactgcaa cctccacctc ccaggttcaa atgattctcc tgcctcagcc |
| 36121 | tcccaagtag ctgggattac aggcacccac caccacgccg ggctaatttt tttattttta |
| 36181 | gtgaagacgg ggtttcatca tgttggccag gctggtctcg aactcctgac ctcgtgttct |
| 36241 | gcctgcctcg gcctcccaaa gtgctgggat tacaggtgtg agccaccgtg cccagccaca |
| 36301 | agtttcattt ttaaaaaaat ctatgggata tgttgaaaag aaatataagg ggaaaaataa |
| 36361 | aaatttgaga attagaaaga gacgtatata tttctaaaat tattgacatt aggtatcgtt |
| 36421 | tcatttaagt agtcaattta attgtgttat gtaacaactc tttttaaaag tcaatattat |
| 36481 | aatgtcaaga cttatatatt gaatcttatg atgaaacttt caaataactt gacaatgttg |
| 36541 | caaaaggaac agagtttttc attccaaaaa catttaggaa atgaaagaaa agtcatttga |
| 36601 | gtctagtggt agagaggacc tgagagaaga gattgcacac tccaccccag ttccccatgg |
| 36661 | ccatttagac agggagagat ggacatataa gtgattctta cagctcccta aacttgtgtt |
| 36721 | tgaaaactgg agaagggata agaactctag tgttgctacc tccctggacc cctacctgga |
| 36781 | tggagtactg gtgaggcacg catccaattc agctgatgat cccWtttttt tttttttttt |
| 36841 | tttttttttt ttcagacaga gtctcactct gacaccaggc tggagtgcag tggcacgatc |
| 36901 | tcggctcact gcaacctctg cctcccgggt tcaagcaatt ctcctgactc agtctcccKa |
| 36961 | gtagctggaa ttacaggcgt ccaccaccac gcccatctaa tttttgtatt tttagtagag |
| 37021 | acggggtttc accatgttgg ccaggatggt ctcgatctct tgacctcgtg atcctcccgc |
| 37081 | ctcggcctcc caaagtgctg ggattacagg cgtgagccac cgtgcccagc ctgatgaccc |
| 37141 | ctttcttaag gcttcctcag agacagccga gagccaggat gatgctgcat aaacagccca |
| 37201 | agagaactag tcaactcagg caatcaaatg ccaccattcc agcagagatt gccagcacta |
| 37261 | gcatgaaact gaccaaacca gagttggaat aagactacag agactccagc atctggggga |
| 37321 | gccctggtct cttcWcgctg aaRagcctga ccactgcaac agtcctgtgt ggctggaaat |
| 37381 | ctgaggtctc tggagaaggc ttgaggaaac cacagaggct ctctcggaat ggcacgattg |
| 37441 | cattttacgt tttctgaatg tgtggggcac atatgctctt aataaaatat tcatatctgg |
| 37501 | aaaaaaccta agtaactgcc tataaacatg tatctatcta aaggtactaa aaagaaatgt |
| 37561 | ttttcaagtt attaaggtga gccgatcctg cttacgttgt tagttattaa agtaagttta |
| 37621 | atttaaattt tattattttt ataccatata tattctatct gcaaaggaaa ggtaacatca |
| 37681 | gtgccagcaa aaccctgaac ctcatttcac gattgactaa cactcaaaca ctcaccacat |
| 37741 | ccatagatga ttctgtgccc ctgttggcat caggagctgt cctgtgccct gcaaggtgtt |
| 37801 | tatcagtatt cctggcttct acccactata cgcaagtagc acccgctcct tgagtggtga |
| 37861 | cacaaaaaca tctccagaca ctgccaaatg tcctgcgggg agcagaatca ccactggttg |
| 37921 | agacccaggg tctgaaaact acaaccgtag aaatccggct cactgcttgc ttttgcaaat |
| 37981 | aaagttttat cagaacacag ccgtgcccat tcatttctgt tttgcctgtg gtcccttcca |
| 38041 | caccacagtg gcagagctga gttcatgcaa cagagacagt gtagcccaca aagtccaaac |
| 38101 | tatttaccat ctggcgcctt atagaaaaag tttgccaccc gtactctagt ttctacctcc |
| 38161 | taagtctctt ctagatccat atttacctgt ctcctgcctc gctgttgcca cccagcccac |
| 38221 | ccggatcaac tgccacctgg atttctgcaa aactcccagg gcactcctgc ctctggtctg |
| 38281 | gctgcccttc agttcatgct tcacagagca tcctggggga cattttccga aagcacaagg |
| 38341 | ttcatcatgt cacttccctg tttaatgcct ttgggtggcc tcccctgtgc ttaagataac |
| 38401 | atgaggcccc ttcacatggc ctgtgaaatt caaagattga ccccatcaca ttccagcctc |
| 38461 | taaaaggtgt ccatgagaga cttccttctg tcctgcaagc agggaaYaca acaaaaccca |
| 38521 | gctctgacct ccagacacga atattcccat ggaMctgtga gcctgggttt accctctgca |
| 38581 | ccgtgtggca tggcatctca cctgcgacag gtgctccgcc agaYgtcaaa cagccacaca |
| 38641 | gctgcccctg tgctgttagc ttctcataca tattcagtta atgtgtcagY tactccactc |
| 38701 | aaccccaaca ttaaccatag gagactctaa aatttagtgt ttgYtgaata gtctcYgcag |
| 38761 | cagggatttt ccagtttttt gataatgacc caaaataaga actgaatata caaaacatcg |
| 38821 | taatctagta cacacatgct tctgtgtgtg tgtgaacaca tctgagacat acatttcatg |
| 38881 | atagaatatt tacctttagg ccgggcacgg tggctcacac ctgtaatccc agcactttgg |
| 38941 | gaggccgagg cgggtggatc acaaggtcag gagatcgaga ccatcctggc caacatgatg |
| 39001 | aaaccccatc tctactaaaa atacaaaaat tagctgggca tggtggcacg cacctgtagt |
| 39061 | cccagctact tgggaggctg aggcaggaga atcgcgtgaa cccgggaggc ggaggtaaca |
| 39121 | gtggccgctg cactccagcc tgacaacaga gcaagactcc atctagaaaa aacagaaaaa |
| 39181 | aagaatattt acctttacta gtaatgtttt ttattgtttt ctatttcatt gtttaaattt |
| 39241 | tggtcacaac tcactaaatt acttcaatta acctactaat gtgttgtgat gtacagtttg |
| 39301 | agaagcactg ccctgcagct tctagcaagg tgacttgatg aattaactaa gtgatgagct |
| 39361 | ggttatgaga atatccaaga aacagttgga agggcctgga tctttagctc tgggtctttg |
| 39421 | gactttccaa tttgttagac tttgtggcaa atgacccgat cctccctcac aaatgccgaa |
| 39481 | tccctcaaaa gggccccggc agaaagagga atggatacaa aggtRctgtt gcgagtgtgt |
| 39541 | ttacccaaat ccttgggctc ctgcaaaaac cctgtggatt gacaacttca agggagccag |
| 39601 | aaatgaccaa agattagctc aaagtaatcc tccagaaaac gactgccaag agaggctaca |
| 39661 | aatgtattcc cccaaaaatg agaatacatg gataaaagca gaaaaatttt taaaacttag |
| 39721 | aatctgtaaa aaacagcgtg gggcactgtg cattggttac cactgaatgc acggctgtag |
| 39781 | ggatccggtg ggctcaagtg ggatctggtg ggttccggtg ggctctggtg ggctccggtg |
| 39841 | gggtgggcac aaggtaccaa ctaccttcca tcctgctgac cttggctggc catctcaagt |
| 39901 | taggcttacc ccgctggcca gcctaactcc tgagcagtcc ccaagcttcc ctgccctgtc |
| 39961 | cccttacaca ccaagatttg actttataaa ttatcaactc acattcccca aatgaagcaa |
| 40021 | acttaacttt gacctgttag tagataccat atgcctcaag gtggtttgta aacttgaatg |
| 40081 | ggaaaaaaat ttctaccttt attttcaata ctttctaaca gacatttaga acacttgctt |
| 40141 | caattataaa cgtaggcaag aaacctcagt ggtatttgct gttcctgtga ctttgctgtc |
| 40201 | aatagaaatc acaaatgttt tcacacatta aagctgttgc aggtatctcg aaatatcact |
| 40261 | cgcactcatc actactccga aatcgtggta gttgttagat ttataccact cgatcttatt |
| 40321 | taagatatta ataaagaagc acatatgaat atacatcaca aatgtggctt ttgatatatt |
| 40381 | gtcaccttat ttcaatagaa caggtttcct ttgtgactct tggtacttca ttcatttaaa |
| 40441 | aacattagtc tgagaaaggg tctgtaggct tcaccaggcc acaaaacggc gaccgtgaaa |
| 40501 | tgggtcacaa cagaaaaagg aatctttgat gaatgcctcc ataggccaag caatttgcaa |
| 40561 | aaaaaattat ttcttcttat ttcagctact ttatttttta atttttaaaa atatttattg |
| 40621 | acttgtattc tccaattcaa aaaataattt gatcttatta taacaagtga aacaatgcaa |
| 40681 | gRaaaagata tacaaaggca gtctatcttg gaagtttgat acacacatca caatggactg |
| 40741 | cataacaaca tagattcaaa taaagtgtta ggaagccagc tcagagaaag acagtgaggc |
| 40801 | tggcaggtag aagaattgct agtgatggtg tttccattgt ggaccaagta agcagtcatt |
| 40861 | cccataagat gtgagcaggc gccctgtgca aaaggattct gtggtcacag tggatgagaa |
| 40921 | aatgctcggt taatcaaagg cacactgcat tttttttaat acactaaaat gcactgagaa |
| 40981 | tctccaaagg agaaaatata atatgcaagg tttctgaaat acagttgtct acaaaatctt |
| 41041 | cctctctcct ccccaaccct ccccaagaac tttcttatat tccccagaac atggtttggg |
| 41101 | aattgctgaa acagggtaat gtaagtttca tcctgacctc agccatttaa aacatgcagc |
| 41161 | caacaatggc tacatctttt Rtttttctct ccttaggtca gtaaggagat atgagatttt |
| 41221 | ttgtcttgcc agaattcttt atttatttac attaaaaacg agaaaaaata tataaccacc |
| 41281 | aacaaaggtt tcactctttt ttttttgtac taatatactt cagactcagc ttttaccttg |
| 41341 | ctggagggag aacttaatta cttacaggtt tttctagaac agtccttagt tcaaccctac |
| 41401 | cacagtttac actScagccc acattacaga attccactgg catttggctc tccagtctcc |
| 41461 | ctctttagag tggtaataca gtgctctcag ggttatacac agagaagttt agaaaatacc |
| 41521 | agccagttga gactagttgg cccctccact cagaaagctg cagaatgcct ttcacccagc |
| 41581 | cacaccaaaa gatgtaagga gcaaagagct cccaccagcc acgcacacaa aggacgtgta |
| 41641 | tgctcacctg gaaatcRgtg gctaaagagg tagcaccctt gcggggacca gaagggaccc |
| 41701 | aaagaggcct gaggaaagtg aaggaaacta gaaaagacag cagagtctca ctcctataag |
| 41761 | taaacttcaa acttgctttt aaaatgatta gcaacacatt ctggtttgtt tatagtctat |
| 41821 | acatgatctc gcagagtcaa gaaaagacag acttccctct gtaacctttc cacttgcacc |
| 41881 | aatgatttcc agatcatcgg tgcctgaagc caatcgcctg cttcaaagag gcatcctttg |
| 41941 | caagagcact tgtgcgttca gagttccagt ggtaccaagg atgacagtga ggcttttgat |
| 42001 | agtgataggc ggcttaatga agagagccag gacagcatgc ttgctctgcc cccactcgtt |
| 42061 | cacttgctcc ctaacagtga atcgggggat ggtctgaggc tctccactcc ctaaccagag |
| 42121 | cccactgagc ccagactgcc acctcgaaaa tgggtgatga ccatcgtttc cttgaccaga |
| 42181 | caggcctcga catcagcctt tctatagaat gccagaaatg gcagagcctg cccttgggcg |
| 42241 | aaaacacttt ttagataact aatcagcaat aatgctgcaa ctgacctgcc tgcaggcttc |
| 42301 | agcggctcca gatctaaatt accctaattt ttctccccca aaattaggca taatattata |
| 42361 | atttcaagga acgcaagagc catttctccc atcattggat accaagctca agatgttaca |
| 42421 | ggaaaaagaa atagctttta ctgctgatat gagaatttta aaagagagac aagtgtgagc |
| 42481 | aaacaaaacc tgtcacatca aatactctgg caaaaaaaaa aaaaaaaaaa agctagaaaa |
| 42541 | aaaatgccag aaatgctgaa atctaaataa agtcatttat ttgttatttt aaccccttca |
| 42601 | ctgtcagagc tactctggca taaggctagt taattctcca gaggtgagct ttattaagga |
| 42661 | cagtgccctt attcttgcag ccattgtttt cttgtcacac agttctcttg attatctggt |
| 42721 | cccttcaagg ggttccacat acaaatgggg tgctgttgat gctccttggg ccgtacctcc |
| 42781 | ttttaaagta tgattttttc agagaatttg attttatcca Rtttccacca tgctaagcaa |
| 42841 | aaacactaga ctgtgagatc ccgaagggca gcagccccgt gaccctcatg gttctggcac |
| 42901 | atggaaggta ttcaaagcat gagatggaaa ttgctttttg aactctgtgc cttaggcagt |
| 42961 | taacatgcaa gcaaagataa gatgccttcg gcaggggagg ctatgacaac caacgagaaa |
| 43021 | aatggaaaaa caccaattgg atgggtctgc agttgatcac ttgctcttcc ttggtcaagt |
| 43081 | tcatgagcct gtctcctgtt tcgaggatac atgcttttat aaatgttaaa taagctaaca |
| 43141 | ctgctgaaag ggcattgcta atggaggtct gtggaaatag agggaaatat ttctaattag |
| 43201 | aaggtatgga ttgggcactc actgtgtgcc aagcactaca gtggaccctg aaatgagtca |
| 43261 | gccacagact cggtcctcca agatccccgc tcctccagct ccattcattt tagcccgatg |
| 43321 | caatgaggct tctttctgcc ttcaggaatt gcttgggcaa tggttcccag gaccttctga |
| 43381 | ctcagaggat tccacagact tttcagccct ttcttctggg aggtctccgt ggccttccca |
| 43441 | ccactgcctc agtcttgtgg tggtgccgct ccccatgggg gcttctccca ccctcccggc |
| 43501 | tgctcctccc ccacctcctg agcccacctc tagaggctgg gtcatctcgg gctgttcctg |
| 43561 | cggtctcttc tgctctctaa atacccactg tccaggaacg cccaccctgc ggtggcctca |
| 43621 | gctcccacct acaagcccag gacttttctc ccaagtccta atcttattaa atatgctgcc |
| 43681 | tgcctgcagg ctgtattcac cagccatctt ctcgccttcg cccaggagtt gataccacca |
| 43741 | ctgagagggc actgccaagc acctatgcag ggaggcccgg gtccctctgg gccctgtccg |
| 43801 | ggccacatcc tcagcatttc tctccgtctc aacagctcaa gccctgtggt acctcttgct |
| 43861 | gagcctctgg ggttctccca gaaggccagc cccaccctcg tctgacccca cagtcctgcc |
| 43921 | tctccagtgc ggctagactg tcttccccag ctgacgccca tctgcggttc cccacggcca |
| 43981 | gtctcaacag tctcctgcct gcacccactc ctcagccttc tccattggag gctcccctcc |
| 44041 | tcagacacac aggcctgtct gctgccccat cgtctctgcg caggctgctc tctcggtgtc |
| 44101 | cctggcccat gcccactcca cgctccaggg ctcagcctcc tctcccgctc tccaccccac |
| 44161 | ccttcgtctc ctgccctcca ccccacccct cctttcccgc cctccaccac acccctcctc |
| 44221 | tcgggctcct gggcaccccc ccgccacctg cgtctcctgt cgtcagcaaa atgcactatt |
| 44281 | tgctcatcca gtgcccacag tttgcccacc ggacggccct gctaggtggg aagctccttg |
| 44341 | caggcaggaa ccccatctcc ttgactttac tttggaatcc ccgcagcctc atacggggct |
| 44401 | gaggcaggtc cttccgtaaa atggaactga atgagcggcg cttaatccat ccgccaaagg |
| 44461 | ccagtctccc ttgggttttc aacaggtaac atgagctttt aaaaatgagt cttgttaaat |
| 44521 | gaagtgccct gattttcaaa gttatgccca cacagaacga cagggcaggg gctcccttcc |
| 44581 | agaatgacag agccctgggg ctcgtctgac cacttctcca cccccagatt cctctcatca |
| 44641 | cccgatgcct ttactttggg actcttccag gtcgttcctc acctcgtgca gcattgctgt |
| 44701 | gagtgggcga caaggaacct cgtggtctcc gtgtgctttg cggggaccac ctcttcgcag |
| 44761 | gggccgccac ctgcctctcc ggcgctgcat ccttagcatc cccactgccc ccggatctcc |
| 44821 | ttcagggagg tgcggcggca gagcggcccc agcagcctcc tcctgtggcc caggcgcacc |
| 44881 | aaggcgagcg cggccacgag ttccggccca gcgccaccca gactctttga tgtgggtttg |
| 44941 | tgtgtgggag agtaaaagat gcacacaccc tccgcaagtc tagaatttgt ggtttttgtt |
| 45001 | atctcacaac ttcatatttg gaagaagtat tttaatgtct tatgagttgt gaataattat |
| 45061 | gtaattctgc cactttctca aaaccaagat ttttcaaacg gactgctcta gtctggagct |
| 45121 | gacatcctta agagaacttt gctttttttc ctgtgggttt taagcaagag tgtgccaaat |
| 45181 | gaattctttc gtccgcttaa aacagttcat tagggggaac catcctgtaa aaggaaagga |
| 45241 | aaggaaagga aaatccttcc ttcttcaaat gaattctcaa aaattacttc tgagatgcaa |
| 45301 | atgctgcctg agtgtttgag ctccgtgaga gagtccactt cccaacttgt tttcagtctg |
| 45361 | ctgttgggtg ctactgaatt tcttaaacat tgtgtttctc ccttatctgt gaattagcca |
| 45421 | tgtgttcttt ggaatctctt cctgggtctt cgttattctt cctcagttat ttaaRaacct |
| 45481 | agggaaggcc cggcgtggtg gctcacgcct gtaatcccag tactttggga ggccgaagcg |
| 45541 | ggcggatcac ctgaggccgg gagtttgaga ccggcctgac cgacatggag aatctaaaaa |
| 45601 | tgcaaaatta Sccgggcgtg gtggcgggcg cctgtaatcc cagctactcg ggagactgag |
| 45661 | gcaagagaat cgcttaaacc tgggaggcgg aggttgcagt gagccgagat tgcgccattg |
| 45721 | cattccagcc tgggcaacaa gagcgagact ccgttaaaaa aaNaaaaaaa agaaaagaaa |
| 45781 | aagaaaaaac cctagggaaa gggtcacgaa gactgcatca tcaaatgaca ccatgaactc |
| 45841 | ctcaagggtc tacccattgt tttaacttac tgcccaaaag gtacttctca ttcgaatagc |
| 45901 | ccccattcta cagtcatctc tatcaatcat catgtcactt gatcatgaca tcgttctaag |
| 45961 | cacctgtaac atcccagacc ctgtctgaag caccaggaat gctgggtgag cgggacagaa |
| 46021 | gggaccacag atcctcaYgt tgccttatca agtttagact ctagtggtcc cgatggctga |
| 46081 | tctcatgaca ttcaaatagg aggcgatcat ttccttaaat acaaacacga gattgtcaat |
| 46141 | agttaggatg tgcttatttt tcctccaggg cataagattt tttttcttgt atgtatttca |
| 46201 | tatccctagc tggagaaata tcaaaagaat ctgaatatca ccatctggaa gttgtgccat |
| 46261 | ggactccagt gggcaggatt cgagctggga ttctcttctt cctctagcag tgtatttctt |
| 46321 | ctgcttaaaa tgagcacaaa cacRtctaat tccctaatgg accaactcag acctaaaagt |
| 46381 | ctcttattct ttgcccgcct tcctctaccc acatggattc ctgggattct ctaaatgcag |
| 46441 | atggcaatgg ggagggctgg ggattcccat caaggatatt tgatcatcgg tgacttcact |
| 46501 | taaccaataa atcaaacgtg ttgaaataca cacatgtcag aaatgtggta tgctgttact |
| 46561 | cactcaaaag tctgttggtc tgtcttggtt gcgattgggc aatgaagccc tagggactta |
| 46621 | tttcggcttc aatagtctgt aaccctcaat caatatttcg ttaagtgtYa gtattcattc |
| 46681 | ctagtatttc cccaagttta acttgttcct tttgaaacga ctggtattct tatgagccag |
| 46741 | aagactgtta tttgaaaatg tttccttttt tttttttttt tttttttttt tttttttttt |
| 46801 | gagacggagt ttcactcttg ttgcccaggc tggagtgcag tggcacgatc tcggctcatt |
| 46861 | gcaacctcca tctcccaggt tcaagtgatt ctcctgcctc agcctcccga gtggctggga |
| 46921 | ttataSgcac ccgccaccat gcccagctaa tttttgtatt tttagtacag acggggtttc |
| 46981 | accatgctgg ccaggctggt ctcgaactcc tgacctcagg tgatctgccc gccttggcct |
| 47041 | cccaaagtgc tggcattaca ggcgtgaaYc accatgcccg gccaatgttt ccttttttaa |
| 47101 | accttcatta tatatcactt tcacatttag aactctcttc gcctccatct agaaatattt |
| 47161 | tcgttttctc tggatgtgct tctgtctccc gctggacaag cctggcactc cgggaagagg |
| 47221 | accctgtgta tcacgacacc tggcacacag aagaagctca ataggcactg aatgattttt |
| 47281 | ttttaacatt gaRgttgttt ttagctctcc cctattagcc gttagttagc agttcacatt |
| 47341 | gtatgtggag tctgtattta taatataaca ttagttctgc cctaaaattc cacaggacaa |
| 47401 | attccatttg gaaacgtcct ctaattgtca tgcagtggtt ctgttcaaac tgcgggaaca |
| 47461 | tttctaaatc ccacagctct aacaggctgc ctgcccatta cagaaggaga cttcaggcgg |
| 47521 | aaattcaaaa cacttgctac tgtgacatct gcccagcagt actaatttac atccatttgt |
| 47581 | atttattatt gtatagtttg gRatgtaata agatgctgcc atcaccccca caagcctctg |
| 47641 | gaatactgag gagtggtatt gcatgtcatg aagcacgcag gctcttcgag tttgacctct |
| 47701 | gctttgaatt cccacttgac ccaggatgag gaagtctgac tgagcagcgg tttcctcatc |
| 47761 | tgtaaaatgg gtataacacc agcacctatc ttgctggatt ggcacctgga ataaatgaga |
| 47821 | aagcacaggc caagccctcc agcattggac acagtcagca tgtaattaat gctagctctt |
| 47881 | cttattaaat tcctttagct taaagacaaa gtaatcagag acacttacag aattacagaa |
| 47941 | gagtttatca tgccgttctc tttgaggaaa acagttaagc agttttaatc cattttataa |
| 48001 | taatgtctta cataatcctt actcagagct ttaatcaaaa aggttgtctt ttaataaaca |
| 48061 | agattcacat ttagactctg tctacacaca caagccagtt ttaaatgtca cccagattaa |
| 48121 | gtcaaacaac cgataattaa caatgtttta catgtaactg gttttgtttg tggacccagt |
| 48181 | cctaaacatt tttttaccaa gtaaccttaa atatgtcatc agatgggttt ttagtagaca |
| 48241 | tatctgaacc taattttgta gcttaaaaat agtatatttt attcactttt tttaatatta |
| 48301 | aatatgtagg atattttaat acttggccaa ttcaagtgtt gataagtaac atctgtcaac |
| 48361 | atttacaata ttatctagct cagttatcca gaataaactg agaatcttct ggatttttaa |
| 48421 | aacaattctc aggccctgat ttactaggag agtttgggag gacctttatg cagacaaaca |
| 48481 | actcatagtt taagttggct tccgctcctt tctaacacaa agataaccgc gaggtggaaa |
| 48541 | aatatcactt tgtgaaaacg acatcttagc aaaacagatc tgtacaaggc agaactggac |
| 48601 | aaaatatgtt gattcacatt accactgact tatatcagac attctatcat tttatctgcc |
| 48661 | tcgagcattt atcaaagtaa actgctgcag agtgcaactg cctccttgtc ggccgggaca |
| 48721 | ggtgttacca catctatcgc ctcgcctgct tgtaaatggg gatagcccgt tcctacagct |
| 48781 | aatcctggca acacaagctc accttcacac agcaggattg ggaggctctg gaaggttcta |
| 48841 | aggccaaaga cagagccagg tctgcttgga agccatctgt gatccccgtt cctacccctc |
| 48901 | caccttgctc atgcctccct ctgcagcgac cagcctggca ccaaaacagc acctgccagg |
| 48961 | ggaacttgag gatttggctg gcctggggct agagctgtcc attcccacca agaggaagag |
| 49021 | gcccacaacc ccagtggcct ctgaggccag cagagcctgt ggtgccacaa gggaacagcc |
| 49081 | tgtgagcctg tcctccactg cgccctgcgc ttctgactca gactaacaca ccaggatttt |
| 49141 | ccatttcaac agcagtgtct gtggtcctct tttctaagtg aaagagtcca agctaaggtt |
| 49201 | ggctagaggg atccttgcag ctccggagca ggtgtagggc tgataccagc cctctgctgt |
| 49261 | tatttgacag cttgcaaaat gtacaaactg gccagataca ggtgtgtgct tgcatgtgtg |
| 49321 | tgcatacaca cacatatctg aatatatatg tgtgtgtatc tacacatcat actaataaac |
| 49381 | taataacttc catgggcaat aaattttcaa atttcttaag gattcagata aggagaatgt |
| 49441 | atataatcaa cacattaatt attatgcata cataaacata ctaatactct taatacgccc |
| 49501 | tttgaaaaaa tatattcaac cagcttaatt tttctaattt tgtttttgtt aaatcctgtt |
| 49561 | ttcttattaa ctattgattg ctaagcagca ggcagtgtgc taagtatgtt acctggatta |
| 49621 | tctggttaat ccttccaata gctacacggt gaagacacag atgtccacat cttctgcaga |
| 49681 | taaggaaact gaggggtaat aataaataat ttgcacaaca cagcacaaag ctgaggagcc |
| 49741 | aacactggac ctgacgaagt cccattctgg aagccaaact cccaactgct aagctctgct |
| 49801 | tgaattctca ttgagcatct Ygcttcttaa aagtttcaac aactcagcag tctataatcc |
| 49861 | aactcgcctt ctccRcaatg ccaaaaggta gaagtgacac tttctttttt ttctttcttt |
| 49921 | tttttttttt tttttgagac agagtctcat tctgtcaccc aggctggaat gcagtggcgc |
| 49981 | gatctcagct cactgcaacc tctgcctcct gggttcaagc gattctccta cctcagcctc |
| 50041 | ccaagtagct ggggctacag gcacacacca ccacacctag ctaatttttg tatttttagt |
| 50101 | aaagaggggg tttcatcatg ttggccagga tggtctcaat ctcttgacct tgtgatccac |
| 50161 | ccgcctcggc ctccgaaagt gctgggatta caggcatgaa ccaccaagcc tggcagtgct |
| 50221 | ttcttataag ggaggtgctt tccctatcca tgagggtatt tagggaacca tataaaaata |
| 50281 | tatatgtaag ttgtataatg gtaattgata gacataacac agttagttaa aaatggcgtc |
| 50341 | aaacaataga ataactactt tccatccttc atagattcaa tactcattta gctgaggttt |
| 50401 | atcattggcc aacatcaggg ttgagctctg ggagggagaa taaaaagtag taggcaccct |
| 50461 | cttctcccca agaactcaca ctgatcatac agataagatg tgtgtggtat aacacttgct |
| 50521 | ttccatgaaa ataaattgat taatcacatc gaaaatagag gaacaaataa tgaaagtgtt |
| 50581 | tcttagggca aaaaataaga gcgctggggg ttttcatacc ctaaaggtag tcctttttct |
| 50641 | aacaaccaaa gaattggcca tgtctactcc tgacatatag ccgtcctcct ttgctaccag |
| 50701 | gaataaacaa caatttgact gagccaaata gcagatgcaa ttttgcatga agctagtaat |
| 50761 | actgtttatg ctttactatt tttaatatgc tataaaaact atttgtcttt tcaaaaaaaa |
| 50821 | atttttttta cagatttaat gccactgcct taactttaaa ccaatggggt agacatttct |
| 50881 | ccttctcttt tcaggggcaa attttggaat tattttaaaa tcagtctgat gcctcacaca |
| 50941 | tcacggctgc taaacttggg tatttgtttt tcagtgcaat gagcttcatg ctgaatactg |
| 51001 | atttgaagtg tggttcacca cacagactta aattgtcctt cccacaactt tatctgagaa |
| 51061 | gcatgacaac ggtgacaatc acaggactag ttttattggg taagactatt ctatgtgcaa |
| 51121 | tttattctga caggctccac aatttttaag tgcaattaaa tcctaagaat ttgcccaNaN |
| 51181 | aaaaaaaagt ctaccacttt ttaaaaaaaa ttttttttga agaaagcaga aaaaacagtt |
| 51241 | gaattgaaca actaattact aactggttgc tctcttgaaa aaaaaaaaaa gatatttgac |
| 51301 | agagcctcaa aatcgatttc attatctaga caggcaactt tctgggtctg agaaagagaa |
| 51361 | ggcttccagg aggtctgcag ccacctgctt cagccactgc tggtatccac atcttcaaag |
| 51421 | cggaggttta gccacaccca gaggggggaa taacacttgc agaaaacaac gctcagaaat |
| 51481 | tggcgtcacc tgattcccca cagcagagca gaccagctgt ctcctgtaca ggtggaagca |
| 51541 | gcttgcacgc ttcctgccat ccgcatgctc actcctccct cacagtccca gaaatggaat |
| 51601 | cccaagaggc cgctcaggca agccagatag agccacccat gtgatcaagc attcagggca |
| 51661 | tgagggcccc cactaccatg gcccggaaaa tacatgcatc aacatgactc atgtcatttg |
| 51721 | cttgcccttg gtgaatttca cgttgcattt ctaacccgaa gctagggtcg cRattgtctg |
| 51781 | gggcgtagag tccctaccac ttctaggtat tccacattcc cagtctctat tcaacaagag |
| 51841 | gctaggctga cactgaagca gctatccagg ttattcttca gaaatcagat atttttaaag |
| 51901 | tgatgccaag tKacacctaa catcatagaa tattctcttc atgtgaactt aattcagtag |
| 51961 | aaaacatctt cggccagaca cctgcagctg gcataatctg gctatctggt gcctgctgct |
| 52021 | ccctctaccc ttgctgctgc cccgacctgc cccatacccc agcccccatt ggggtcgacg |
| 52081 | gtttcccaga gatactcagc cttttggtgc tcctctctcc tcctggtata ccccatgcat |
| 52141 | taagcattga tcctcccatc ttgtgtgccc ccaagacatc taccactgtt aacactaaaa |
| 52201 | tggccttgcc tgtctcaccc aatttggctc cgaactcatt gaggaccacc tYacacactc |
| 52261 | cccagcacct cacacagtta ctggtacaga gcaRgctccc ggggatgttt gctgaaRgtc |
| 52321 | tttttttccc ttctgagctg ctaaaacaac aggcattaaa cagtcacctg ggccacattg |
| 52381 | ttgctgtgaa atgtccttct ctgcagatcg taagccccgt gacgtgatgg cagaaaccgc |
| 52441 | gccctccttt tgtgccctaa tacctgggcc agtgctctgg gcctattaga tgccaataaa |
| 52501 | tgtttcttga ataaatgaat taaagacaat actgatacag acatcctgag agaaagccga |
| 52561 | aaaggtctaa aaatgaaggc attccaacat ccatgtgcac agaacattgg gaaagctgtc |
| 52621 | catcagcgtc tctcttttgt gacttcctat taaggtgctc cgcagaactt gaactgacct |
| 52681 | attgcagtac agcacagtac tagggacaca ggccaggtgg aaagagatta cttcatctgc |
| 52741 | ttgccatgca aagcagggct gaggatccga ggcctcaaca tggagagtgg gaccccaggg |
| 52801 | ccctcacccc tcccttctca ggccatttgt tcctttttcc tggatgacca cctcccacct |
| 52861 | gtcaccccct gcctttgcct tccacttggc caatgctaaa tcaccctctc agactcatct |
| 52921 | caggtaccac ctctccaagg acgcgttccc tgacccctct gggcctggcc tggtgtgaat |
| 52981 | gcatactata ctgttaccct cactggacag agacactctc ttcctttttt tttttttttc |
| 53041 | agacagggcc tcactctgtc gcccaggctg gcgtgcagtg gtgcgaacct ccacctccca |
| 53101 | ggttcaagtg attctcctgc ctcagcctcc caagtagctg ggactatagg tgtgctgcca |
| 53161 | ccacatccag ctaatttttg tatttttagt agagacaggg ttcctctatg ttggccaggc |
| 53221 | tggtcttgaa ctcctgacct catgtgatcc tcccacctca gcctcccaaa gtgctaagat |
| 53281 | tacaggtgtg agcagccacg ccaggcctgg tcacttcctt tctgagtatc tccaccgcta |
| 53341 | ctgagtgagg accttgagca gtgccaggat ttgctgttgc tcctgcctcc caccctctct |
| 53401 | tgaatcctgc tccctttccc cctgcctggg cctttgccac ttccccagat tttaattatg |
| 53461 | caaaattaaa tagctgcttc ataggcgaac agctggatac aacaccttgc agtcttcagg |
| 53521 | ggaaatggtg caagcataaa aaacaggaag agcaatttaa gattaaaata ctgctttgag |
| 53581 | gctggtgtgg gcactgtctc caacagaggc tgacacttgc ctgctggatt taaacctgcc |
| 53641 | catggtcagg catcaagccc cccatgccag gcctagggga gacagaggag gtgagatggc |
| 53701 | cacaccctca gccacctttc ctactacttt tggcagctcc acgattggca ccagaatggc |
| 53761 | acttattgga gggttttctc cctcgcagcc ctcctaagaa tcgcctgctg cctttcctcc |
| 53821 | tacccattct aacccttcaa acccagggga tgctgggctt ccgaccctct tcccctttta |
| 53881 | taagtgtaag gtgctatccc tgggaggcca atgctgttgg ctgcacactt atagaacccc |
| 53941 | aagttatttc caggaccccc taaaaatgtg caatgctgaa acaggactgt tttactttga |
| 54001 | gcaccagatg aattctggaa cagtcaggtg caaaactgaa tcctggcatg gcgaatcatc |
| 54061 | ttccccatgg cttccatttt aatttcagaa gtgcattagt ggaggctgag aacttgcatc |
| 54121 | caacacaaca tgaaatctaa tgaggaattc cgacattgcc tgagagtgca cacagcccat |
| 54181 | gtgcatttca tttgcctgta tgtgccctat aaaaaattca accactttgg gaggccgaga |
| 54241 | tgggcggatc acgaggtcaa gagattgaga ccatcctggc taacagggtg aaaccccgtc |
| 54301 | tctactaaaa atacaaaaaa ttagctggtc gtggtggcag gtgcctgtag tcccagctac |
| 54361 | tcaggaggct gaggcaggag aagggcgtga acctgggagg tggagcctgc agtgagccaa |
| 54421 | gatcgcgcca ctgcactcca gcctgggcga ctgagcaaga ctctgtctca aagaaaaaaa |
| 54481 | aaaaaaaaat tcaaccaaat tcaaacgcac catcttttat aataaaaatc tctctaatat |
| 54541 | ttaaaatRta catcatactc ccatttaaat cacaatgctt actattttta cccaaagtgc |
| 54601 | ttctatttct tgaaagagct gatgtttact agaattgaac atgggtcatg caagaagcac |
| 54661 | tgagtcaaga ttggctggga ctgcggaatc ctctctctcc tgcaggaatc ccatccacac |
| 54721 | gctcctggtt cactttcatc atctgaagga cctcttcgta cctagattct acttgttatg |
| 54781 | tgcagtttat tttggttcta aaggtcagag gcaaatatta ataaaggcat ttgcagcctt |
| 54841 | ttaagcttgg aagatattcc tggcagggcg aggggcaaga catggcatYa gaggtcattc |
| 54901 | accatccccg gcactgtgtc ccgccggcca caggacccct gcattcactc ccttacctag |
| 54961 | tagtaggata tatggaaatt agcctgggaa atgttgtatg ttagcatagt tcatgtttaa |
| 55021 | tatactaaag catggccttt agtccataag gttttatgaa ttacccgaac atatacaagc |
| 55081 | attctttaaa caaataacac ttaaatacca gagcgacaat taaaactgac caatggaaag |
| 55141 | gatctggaag gccctggaac aagtgccttt tcttttttgt tgtttttggg tttgtttgtt |
| 55201 | tttgagggaa acaggagagg ctctgaaatc caagtctacg gtctcttaca tgaaagtgca |
| 55261 | aaactccaaa aagctctaaa aaccaaaagt tgttactcaa attttgcaac ccaccctgac |
| 55321 | atgcggatat ctaaggtctt cctgtcactc cacttagagt gaccattcct atgttcMcta |
| 55381 | tccaaatgcc catgcaattg ggaagaaagg aatcccaggc tggagaagtc tagaatatcc |
| 55441 | cattacagtc ttgatactgt attcattttc tattgaaaga aaaaaaaaat ctcaattYtg |
| 55501 | aaaaacatcc aaccacgcag gttttgcaga ctggtactat tagagtctga cataaacagg |
| 55561 | ggcctggcat gtaattaagt gagaaggtac ccaagctaca ttgaagaagg acggtgactc |
| 55621 | gYagggttaa aactgagagc ctggctctga tccaaagctg tgtttaaaga cgtgcaaaca |
| 55681 | taccacaccg gttcaccgat catcagccga agctccaata tcaattctgt gttaaaccat |
| 55741 | caatctaaag tggatatttt tgttattttg tttatctttt ttcccccatc aggacaccYa |
| 55801 | gtgaaccaat ttcaaatgac acctgaacct acatattttt cttaaactac tgaactggaa |
| 55861 | caaaatcaaa acatcaaaat attctccaaa agtggcggga atggaatcgg gatttcattt |
| 55921 | cggtttaagt cccaggttga aaagacacaa gatggtcagg tcatctcatg gatggaaaga |
| 55981 | gcttaaacca cccaYccatt tttgaaacct tgacctcgag aagaaagaga gaaagctttt |
| 56041 | aMgtttttaa aacagctgga aaatcatacc atctgtgcat ttttacccag ccatactcca |
| 56101 | aacccagctc tctattttat atacctgttt ttttcttgtg attatctcac tgttccttgc |
| 56161 | aagaatgagc cgataatttg atactccaga gggaagttca atttctcagc tgaagatgaa |
| 56221 | gactcagggg tggcctcctt caggaattgt aaagttcagg ggagaacggc aatccatttt |
| 56281 | tatatgctaa tactttgtat ttagttaatt tgattaagtg gtaggaaatg ataccttgct |
| 56341 | cRtagatgga tcgttcgcac atttgcagta aaacctaaat gccaaatttt cctccttcaa |
| 56401 | gttcttgaga gagttatttt catcactttc atttcttcct gtaccacagc cattgctcat |
| 56461 | ctctttgttc cctgaagagc tttaaaagta gctgtgttca tgtcattttt atcttgttac |
| 56521 | tttaatcatg tgcttattga caagagcgga atttgtcatc ctcttccaag caagccctgt |
| 56581 | caccagtttt tcagcgtgat tcagcataag tcatgctccc ctgaggaagg gacactaacc |
| 56641 | caacccaccc aggaacacaa ggacgtcact ggagaggaac ttttcccaaa atccccagga |
| 56701 | cctgccttgg tgcagggcaa cttcaaagga gagggtcttg tgtgaccagg aacagataac |
| 56761 | ctgaggaggc atcatccttt ccaggagcct tcgccatgac atggcagcac tgttagaatg |
| 56821 | tcttgtttgt ttctccagag tctgttcctt ttcttaggca agtccagaat atccaaagca |
| 56881 | gacagaactg tttggatttc tggatttgct tttgtttgtg ttttctctac tttctattgc |
| 56941 | agtccaactg agtcacattc attttttaaa caacttaaga ttccatgtca tttaaatagt |
| 57001 | gttatgttgt ttgagaaagt ccagtggcag gtccggtgag ttcagtgaag aagcatgccc |
| 57061 | actgctggcc tgtggcttcg gtgaacgggt gcattctttt tttttttaag acaaagtctt |
| 57121 | gctctggcac cctggctgga gtgcagtggc actatcttgg ctcactgcaa cctctgcctc |
| 57181 | ccagttcaag tgattctcca gcctcagcct cctgagtagc tgggattaca ggcatgcatc |
| 57241 | accacgccca gctaattttt ttatttttag tagagacagg gtttcaccat cttggccagg |
| 57301 | ctggtctcga actcttgacc tcaggtgatc cgcccgcctc cacctcccaa agtgctgaac |
| 57361 | gggcgcattc ttaactgtgt ttctccttcc gacttgctga aatatgagtg atttataact |
| 57421 | cagcatccta ttgactgacg gatggcctgg tttctgtgtg ctgggtgctg acagagcccc |
| 57481 | cagaagtgcg taaaatgata cttgctcacc aggggttccc attattttct cctcaaccag |
| 57541 | aaacattccc agagacatgc cacacagtga gaaataaaca tggtgcaata aaacacagac |
| 57601 | ggtaccgcga ggatcctctg tgaggagcaa gaggtggaga cagtcccagg ggagtccaca |
| 57661 | gtgggtgggg tcaaagtggg tctgagtggt gatgggagag ctgggtctga gaggtgccat |
| 57721 | ctggacaggt ggaaagaagg gtggaaggca tttcaagcag ggaaaaggag gtaagggaag |
| 57781 | Yccagagaca gtgggggaaa ggctggttag ggagtcaggg aagaactcag tgcagtcaag |
| 57841 | gaggaggatt ttgaaggcag ggctgagaag tgcctgaggt tggatgtgga gttgggagtc |
| 57901 | ttgacctttc ccacccaggc cttgatgtat cccagctgag aagatgcaag gtcagcaggc |
| 57961 | aggctagggt ggcagatgga caMaaaacca gagaccaggg accaaggagt agatccctgc |
| 58021 | gacagcagga ccaggacagg caatgaaatt tgagaaaaat ttcaaaataa ttgatgacta |
| 58081 | agagaaagca gtctaaactc caatattcag tttttgcaca cctatcttta atgatctcct |
| 58141 | tacagttaga aaacagtcca ttttccccat aatcggtagt cgtttcatta gcatctaaat |
| 58201 | tataattcct taattataag cctatcacct attctgcttt ccaaaaatta atccagaagc |
| 58261 | tgtcaaatac aaacttatgt ttaataaaat gaacatctgt gatcacacac caggagcaaa |
| 58321 | atcctaattg ttctaaaccc ggcactttgc tttgacaacc aagtaccttt tttttaaata |
| 58381 | ctttatttct ttaaaacacc tgctttcttt acatactgac ttttaaagga agagattaaa |
| 58441 | ttttattggc aggaacagaa gttagtctct aaaatagcaa aggaagaact atgctttata |
| 58501 | gaaaagttgc ctgctacatt aaaaataaac cagaatatga atcaaaatcc acaaatccaa |
| 58561 | caggggccaa tccccaattt tcccacaaag agagccgaca gtctgataac ctcagctctt |
| 58621 | tggaattagg ctgttggtgg cggctgcagt ccttgttccg atggtagccg caccacccca |
| 58681 | gcccaggatg gctgtatttc cattagaatt ggctgtcctg ccctctctgc cttcgccacc |
| 58741 | ccccttcgcc caagtccggt tattcagtga gttataattt ggcactaaaa ctcaattaag |
| 58801 | ctgaaacgtt ggtcatacgt ggtgtttttc ccagcaaagc ataatttctt tgacccattt |
| 58861 | tccaaatgtc agtagatcga ttttaaagca cagagaggta aaagtYtgtt taaaaatcca |
| 58921 | gctcgcattt ctagaaactg gctggaattt ttttttttta atttcaaata aacagtcagg |
| 58981 | atgtggtgtt actatattaa agcgcttcaa gttttcatag cgctcgtcaa acagagacct |
| 59041 | cggaacgctc tgcagacatt aattaatgtt cgctccggcc ctggaagcca tttcaccaat |
| 59101 | ggaaattccg atgagaaagg aagaaacgag gggttcaagg ccgcgccgtt gtgagtccgc |
| 59161 | tgcggaacta ggaactgaaa ccccgtggtt cccaatgagc cagcgaaagt ctgtcgaagt |
| 59221 | attgagcacc cctattgaag actgtgggag ttttggYggc ttaatgggat tgtgttcggt |
| 59281 | ccttcaggct ctcctttaaa agattccatg gcaccaacga gactccaata aaagggcctc |
| 59341 | tctaattgct ttctgacttt ctatgaaact ttctaagcta tgctgacatc ctcgaataca |
| 59401 | actatggaat taaagaggag agaaaggcga ggcacagtgg gtgcgcaaag aaaaagaggt |
| 59461 | tctcagcgga gcgtgtgtga ggcggggggt tacgggggag gggagacggg gcgggtgtga |
| 59521 | agtctgtgcc gggaaaagaa aaatatgagt gttctctttt gtgtgcgcac aaacagccga |
| 59581 | gcgcggggag gtcgggcccg gggtcacgcg gctccgggct gcagctgccg ccgtggggcg |
| 59641 | gggggtccca ggcggccacc gggcgtcgcg gccgcctgcg gcatcagcat tccggcggcg |
| 59701 | cgggcggccg ctgtcctcgc gcgaccccag cccgtccaga gcgcgccccg gcaccgaccc |
| 59761 | tcggggcgag gcgagcgcag cccccgctgg ccaacaaggt aagcgcggcg cggggagcgg |
| 59821 | tggccgggag cctgggcgcc aggcgacccc tcgacagggc gcacggaccg gttctccgcc |
| 59881 | gcgcgggtcg atgaccgacg gggaggcccg agtctggccc cgacgccctg gcccggtgtc |
| 59941 | cccgacgcgc ctggagcccg ggagggaggc gcgggcctgg cctgggccca ggaggatgga |
| 60001 | gcccgactca gggccgtctc tccccgcttg ggggtgctca ggagggccca gggtacctgc |
| 60061 | gcgggcacct tggcggcggg tcaccctccc tccgagccac tccgtccgtg ggcagcggcc |
| 60121 | ttaccaggtc cgagccggac gctcagcgtt gccctccgcg agcccccagc ctcccctcga |
| 60181 | gcgccgcggg agccccaggt gcacacacgc aggcctgggc cgggcctcca ggcctctgtc |
| 60241 | cttcgggcgg gctgggaggt ggcctggcga tgcccgccga ggggtggaga gggcgaagcc |
| 60301 | agggcggcgt cggcgaggcc atcaaaggcc gggggtggcg gcccgccgcg ttgcccatga |
| 60361 | ccaggccgag ggcatgaaag gatctttatc ctccagtgac attgccaggc cggcgctttt |
| 60421 | ctgcggcctc cccctcccgc ggccctaatt ggggccggct gctaatgtga ttaaaggcca |
| 60481 | gttaaacgtt tctccgagca gggggtggga agaggccgag acccctgccc cctccccagg |
| 60541 | gtcacgccag gcccgtggtc gggggtcggc gacggtgcca gcctgggacg gcctggccag |
| 60601 | gtgcctgccg attgttcacc cccaggcggg cgggccggcg ggctcccggg gctttatgta |
| 60661 | agccggtggg ctttgggccc gaccggattt agggattctt ttcgcacagt ttccattttc |
| 60721 | tggcctttgg aactgcgccc taagccctcg gggccctccc cctccggtgc ccggcgctgg |
| 60781 | cggggcccag ccacgggggc gggctggggg tgccaggctt tgggggtctg gagggcctga |
| 60841 | agcaggtcgc gttgggggtc tcactaccta agacagtgac tcaggacagt gggacctggg |
| 60901 | tgctgtgctg cggccaggca gagccKgcga cccgcggcgc cccccgcggc ctcggcgaca |
| 60961 | gcccggttcc cacgtcggcg acggcgtggg tggtcccgcc cgcccgggag ggctccgagt |
| 61021 | cttggccgcc cggggcgcgg ggcctgagag cgagtgtctg cgccgcgctg cccgccgcgc |
| 61081 | cagtttccga ggtcgcccgc gggctcatgc cctgcgccct ggggccccgg ggaccttctc |
| 61141 | caagccggcc tgacctggtg gaggctgtgc gctgcgctcg gggaaggccg ggctggtgcc |
| 61201 | gggggcagcg ggtcccagct ctgaagtgcc agcgccggcg ccccactccc cgcctgctac |
| 61261 | ggtgctctcg cccggaaagg cccgaggccc tggaaggagc cccgcggggg ggcgccgtct |
| 61321 | ggagcagccc tcaggggcgc cgggctgccg cgacgcagtg gctgtaattg cttcggggta |
| 61381 | cctggggatc gatggcgtgg cccagccggg tgtctgcgcc tctgaggttg caaatctgga |
| 61441 | accagttagg atgcgggagg cRgataataa aaagccaaat gggcgccagg tggaggagtg |
| 61501 | agctccgcac gttccatcag gtggcccggg ccgcgcgctc gggcggcggc agagtctctg |
| 61561 | gggcactcgg ggcgggcggc agcaggtggg acccgcggcc cgacccgcgc tagggccgcc |
| 61621 | caccccctcg gaccccggcc gggtctctcc acagcctccg tgcgcgtccc agaaggaaaa |
| 61681 | tgctagcaaa ccggaaggcc gcacggggcc gagccggcct cagcccggcc aggagaggcc |
| 61741 | gtcggtgccc cgaggaggaa ggggacccgg cccgggacgg ccgccccgcg gtacccagga |
| 61801 | aggacgcccg ccgttcccag caccccagcg tctgaaaaat gaatgtgaac tccgcaagcg |
| 61861 | tgctcccgcg gacattttac atgtcggttg ttcacatttt aaaaatattc tctgaaatag |
| 61921 | tctgcagaac tttttcacat ctaacaaatg tcagattgaa cagatcagaa gataagaaat |
| 61981 | gaacatgaca ttttaattca agtagagaga aagcagacgg tatttttaaa ataatatttt |
| 62041 | tgtaaaaacg tattgtggat ctaagatttt gtcatgttca aaatgtataa ttttttgata |
| 62101 | tttaaaatac acattttgta cattttcaac caaaaagaca gggaaaaatt taaaaagcaa |
| 62161 | aagctggaaa ccaagaaaag cctggagaga aaatgggccg taaggaacca gagcccgtct |
| 62221 | ttctccacac catttttttt Nccttcctaa tagccagaaa gaaaaagaaa aagaaaaaaa |
| 62281 | aaagtgaaaa taaaataatt tcatattaaa ctaacacaga gaactaatta atttctttcc |
| 62341 | agatgggttg aagggacctg gctgttttag tgtgctcaga aaatgctaga ctcaagttgg |
| 62401 | ttttctttcc ttctgctgct cagagaatct tacgaaaaag taaacaaccc cagcctaacc |
| 62461 | atttttttat cggcttattg aacacagata aaattagaat ccttttttat ttgagataaa |
| 62521 | agacacacag gaaaagacta Nttttttttt taaccaaggg aatatctggg gtttttttgt |
| 62581 | atgaaactgt aacatttaac taacatgtaa tgagctgaac gaccttgggt ttgtttcact |
| 62641 | ctagctcctc ctgaaatctt ccattttttt ctgagatggc ttttccagtg atcttgtgtc |
| 62701 | agtaagagag gaatatacag tgcagcaaat aatttgtgca ttcgtggtca atacatttct |
| 62761 | cagtcaaaaa atcaaaaaca ggtcaatggc tggttattag gaataacctt tgaaccgaac |
| 62821 | aggaaatggg gaaaatacct gtttgttgac gcttgcacgc ccaggaggag aattgaggtg |
| 62881 | cagttagttt cactgtgtga gggagagatg ccgctctgtc cagagggttt attggaatgc |
| 62941 | caggccccag gtgtttgggt gtgtctatgc agacagttag tgctgacccc tgaggatttt |
| 63001 | gatgtgtata atcatgccat aagaggaagg caggtgggtc aattgtcagt gggagcacag |
| 63061 | gagatgctgg ggcatgccgg agcctgggca gtttctatcc tgataccttc cgtgtccatg |
| 63121 | ttttagtccc atattgagga catgaaaggt ccattttatg tttctctcct ttaaaaactc |
| 63181 | cccagtggcc tactcggcag cattgctggg gacccactgg tcatttttca taaaaataag |
| 63241 | accacagtgt ggaccaaaac ttgggatctg agatgtcctt gagctcacct gtcacatcga |
| 63301 | gtcaccacct ggagaagaaa gatttgctga cctcctacag agccaggcaa taggacgttc |
| 63361 | agcctcagtc ctgattacgg gtgggcagtt gggtttatgt gtgtgtgttt gggttttttg |
| 63421 | ttgctgtttt tgttttgttc tgtttttctg ggaaaacaaa ggtccctttg ggggtacctg |
| 63481 | ggctggccgg tacggcaaac aggaaagcca gccaggtcag gaggagacgt ggcttcacgt |
| 63541 | tggaatgcca gtttgctttg tgattgttta ttacaactga aggttttgtg tcatctagcR |
| 63601 | ttgcccaaag tagaagccca ttcccaggat ggatgaatct gtggtaagag ataaacacaa |
| 63661 | gaaacaaatg cccaggggag gaggagaagc agattgcctg gggcaggtaa gagggctaca |
| 63721 | cctggaaatg catctgctgg ggttctcacc cacaccagaa tctctggtca gagcagctct |
| 63781 | ctttgaagaa tgccagacaa acagaaacct ggccacaccc ccaccagagt tacctgattg |
| 63841 | aaacacgaat ctgatcactg aaagccttcc tgcatccgtc tgctgcacgc aggaggcagc |
| 63901 | tcggcctcct tccccagtgg cctcccagcc ctacctctag aatttcctgg cccctgccct |
| 63961 | cctgccaccc tgatatcYct taStccctaa gacacttgYc cttgtcgccg acacgggccc |
| 64021 | agcggtgaga gaggcgctct cNgcccattg cccgtgaaca cactgcgttc agggcttctc |
| 64081 | agagcaggcc agcctggtgg agctgctttg gggcctagct ctgggggtct caggagtacc |
| 64141 | ccagcctgca gataaggaac cctatgcaat gtataaggaa aggaaagcat ggcccatgtg |
| 64201 | agcacagcat tagggccttg gaactccgtg tttagcaccc cccatgctgc agccaaacat |
| 64261 | tgacacccca agttcYagga ccccatgaga tggtgccttg cctagccagc tgcgttgatg |
| 64321 | tgtgggaagg ggcatgttgc ggaaatcctc cagcaggtcc tggctcctgt cctgtgccca |
| 64381 | gcactggcgt tcttgttcag agaacaaagg cccggaggcc cctgcagaac caagaaagcc |
| 64441 | tgaaagccgt ggctttcaaa gcttgctgac cgtggctggg gtaagaaata cattgtgcat |
| 64501 | ctcagcccac ggcaagccag acaactatgt ataaaataat taattaacta tacctagata |
| 64561 | accaaaacac aatttttgca aaagaatggt tctcttacta gacgtcatga atgctgatct |
| 64621 | ctgttcatcc tatttctttt ttccaaataa tggctgccac ccctgaaaca atcccatgat |
| 64681 | cctaatgggc tgcagcgtgg agcttgcaga ccacccttta gagctgaggt tgtgtgggtt |
| 64741 | aagcctgagt ggagggagac ccgcagttcc ttcctgctgc atctcctgcc cccacttcca |
| 64801 | gccctggagt cagaacctcc tgctggaatc ctagccacca cttcctacta tgacattgag |
| 64861 | aggccacctc ccctcctgag ctggagcctc tgcttcttcc tcttagaatg aggaggacaa |
| 64921 | ctccctgcta gggtcttaga gaggattcag cgagatcatg taccagatga ggtcttgact |
| 64981 | caatgtgtct gcccaggcta gcacctgaca gtcatctgtc acttttgatc ctgtgcctcc |
| 65041 | ctggggccca agatgtcttc tcccctaccc cccagtagcc ctccagtaaa atcctcctcc |
| 65101 | ccccttccaa ttccactcca gtcttctcct tgggccagct cctcctcaag ccccactgca |
| 65161 | gattttggga ctcacagatc tgtgcagcgc ggattaaggc ctgccaccct ggtggactct |
| 65221 | caggcctggg ctctgcctgg ctcgccggca tttatagaca catgtctgat tgccagttag |
| 65281 | ccccgatcca acctcaacag cactgtggac acagaggctg tgcttcttcc catttgcaga |
| 65341 | taaagtatat ggggcccagg aggtatcatc atgtgaccgt gagtcgcaca gttagtggca |
| 65401 | gagctggggt aggctagctc tcctggcctt caaagaccat catgctcatg gcccagagtc |
| 65461 | cttcctcctg gcagggcact agggcccttt ctgacaggct cgcagcaacc agatttgttc |
| 65521 | cctggctgcc cacctccacg tcctccaccc tgggagagct gagttcgttt tcctttgact |
| 65581 | gtttttttaa tgcttccctc aggaggcagg ccacagtctt cctgcaccac cctcacgggc |
| 65641 | tctcactgct ggagcaatga ttactcacag atttacccaa ggagagtgtg tacttggaat |
| 65701 | ggctgtccgt gtgttccctg tgcacacaca cacgcatacc cgcacataca ctgtggtgac |
| 65761 | ctgacacaca cacttccgtg atctcacaca ctcgtgcaca cacacacact gccctgatct |
| 65821 | cacacaaaca cacaaacact gccctgatgt cacacacaca cactaccctg atctcacaca |
| 65881 | ctcatgcaca cacacacaca ctgccctgat ctcatacaca cacacacaca ctctgcccta |
| 65941 | atctcacaca aacactgccc tttgccctga tctctctctc tcacatactg tcctgatctc |
| 66001 | acacactcat gcacacaaac acacactgcc ctaacctcac acacagacac acacacactg |
| 66061 | ccctgatatc atacacacac acacagacac acacatactg ccctgatatc acactcatgc |
| 66121 | acacacactg ccctgatctc acacactcat gcacacagac acactgccct gatgtcacac |
| 66181 | acacacacac acgcacactg ccctgatctc acacatacac atacacactg ccctgatctc |
| 66241 | tctcacacac ccactgccct gatgtcacac acacacacac acacacacac taccctgatc |
| 66301 | tcacacaRtc atgcacacat acacacactt ccctgaccct atcactctgc acattggagc |
| 66361 | taagctcatg aaggctgctc atgcatttta tttggcaagt ctctctcaca tctttaatca |
| 66421 | cactagcctg cccactgcca atcaataaag attaggaaat cgattgctaa cttactcttg |
| 66481 | ggttagataa taaaattgtt tgaaacatgc acctggagga atttatctgt gaagagtcct |
| 66541 | ttcagagttt gtttcaagct ttgcaagtct ctcatggctc tgaggtcggg gaggctgtga |
| 66601 | gggatcctcc acgcccagca gacgtggaag gagccgacgt tcttacggga gatggacaga |
| 66661 | cggtcagcat gtgaatagtc ccctgcatgc aggtgaggac tggggcagtg cagagccgca |
| 66721 | gcagggatgg gctatgcctg ggagccgcca gctctggatc aggtggccag ggacggtcat |
| 66781 | gctgaagagg aaggctccag tgaagctctg gaagaatgtt ctggtgcctg tatgtacaaa |
| 66841 | ggccccccta attcaggacc tggccctgta cagatgagga gcaggaagga gccagtgtaa |
| 66901 | ctgaggccag tgtggctgca gcaaggggct ggaggcgtga gaagaaacag aaaagaacgg |
| 66961 | agggccattt ctgcctcccc agtccggatc atcctcctcc tccaccccag agccccaggg |
| 67021 | tgctgagcct tgcgcccctg cccctcaagg ctatggaatg tcattgccac tcagcgtgaa |
| 67081 | ggttctggag gggacctcaa acaccaggcc cctctgccca gagagcccca gtggacccgc |
| 67141 | tgagccaggg acctgctcct tggagccaac caccttgtga cttgtgaatt gtgttgagtc |
| 67201 | ccgtttcctt tttccctttt gtttattgct ttgtgtatct ggcagcttta tcccaaagct |
| 67261 | gctctcacgt ggtccctctg tcccatcctg ccattttcct ccctctgggg ccccttgcat |
| 67321 | gggagactgg gagcagcgtc cacccgccac ccagcagagg ctcaggtcct ggagttaaaa |
| 67381 | gagaagcaca ggcaggggaY gctctccaaa ctgccttcag cccgcatctt tgaactctgc |
| 67441 | agtaggaaga ctcctgggcc cccatgtccc catgcatatg cacctgcatg catgtgacac |
| 67501 | acttctcggc ccatggagaa gactgtccgc acgggggtgt gagctgccca gcacccactg |
| 67561 | atcaattgct gcattcccag ctgcctttaa gctggcatgg ggcgccctct gaggcacgcc |
| 67621 | cagcccctgg ggcctagact ctggtgttct ctaggaactg tctatctctg tgtgtgggag |
| 67681 | ccacagactt gggctttcag acccccacaa tggatggctc aggcctatgg ctcaccccca |
| 67741 | gcatctgtga ccaccaccac tgcccacctt ctttgcctaa gcattacttc tggggggcag |
| 67801 | ttcttcactt atgtgtgcaa gccagccttc tagaaaactc tttgctctca tgaccaggca |
| 67861 | ggccttgcac agtccagatg gtgaagccca gttcctacct tccagtgagg agcacctgac |
| 67921 | ccttcagggg cgatagacgg ggccctgcac agatgggaca ggacaggtgg ggtgcacaga |
| 67981 | ggatggcagg tgccctctga gaggcagggt cggggcaggt cgggcaggag cctggcatct |
| 68041 | gtggctgact gtttggcccc tcaaggcctg ttttgctcac agttttgctt cctccttgct |
| 68101 | ctctgttttc cctgccacct gaacttggtt gcatgctgct ttctgtccat tgccctcagc |
| 68161 | ctctcacctc agtcatgacc cttaagccag cacctccccc atgcgtctta ctctgaggtt |
| 68221 | tcccaggagc atgagcctcc tccagtgtca ttgcggctgc tttgatctcc ttaatggttt |
| 68281 | tgcttgagtg tggattcata ttcagactct tttagggaca caaagacccc caaagtggat |
| 68341 | gaatgcccta tggaggcaga tgcccagttg ggggcccgag gtctggctgg cctgtcctcc |
| 68401 | ctgcccaggg tttgctgaac accccagcca catgctccag gcacacaccg aagcttcagc |
| 68461 | tcaagattca cctcggagga gggcatctag tccaagccac ttcgtctaag aggcgaccaa |
| 68521 | ggtggccagg acttacttgc catgagacct tggcaagtgg acaagtgctg agagtggtgg |
| 68581 | gaggtgacag gcttctctac cagcactggc aagcacttaa tccaagcccc gaggctccag |
| 68641 | ctgcaaactg ggacgggaat ctgatgacac ccaccctaga gggatggggg taaatgtaga |
| 68701 | cgacataatt tccatatgtc tgcattgagc gttcacatac atgagcgtcc tcagaagcat |
| 68761 | tatctcctca tgcttctcca aggaatgtgc ccttttctta accctggttg actgagtcct |
| 68821 | accgaggaag gaagggtttc aggccacacc ccagggatag cgctggatgc ccccagccag |
| 68881 | gccaaggtgg tcagatttcc ttgcaagagt ttgagcaaag acacagagat tgaattagac |
| 68941 | tgagctgggc ctggaggctt gaggtcctgt cgacgaggag agcgatatct cagtgtatga |
| 69001 | atctgtgggt tttctgtgtg cccttgcagt cctggggacc ctcatggccc ttggtgagcc |
| 69061 | cacattgaag gagaagaacc aagaggcggc agagaatcta gattcaggct gcagaaagtc |
| 69121 | ttgctactgc agaggtgaga cgaggccagg gcaggaaggg agcaggggtg gggtgcagga |
| 69181 | ccctgagtcc cctagaggcc acggtaggac atcagcagca ccagggggag tcaccaaagc |
| 69241 | taagtgccaa gatgYggtcg acYgaggacc tgctcctggt gcccgcccct tcagcccaga |
| 69301 | gacggtgtgt ggtccgtctc cacccagcac aaccgtccta aatacctgaa tgagtttcct |
| 69361 | tgagctcctg cagcagagac ccataaaatg cttaaaggag cagaaatcta ctcccttgct |
| 69421 | gatctggagc ccagaagtct aaagtcaagg tgtcgacagt gtcgtgctcc ctctgaaggt |
| 69481 | tcctgggggt gcccttcctc acctcctcag ctcctggtgg ccctggacat ccgatggctt |
| 69541 | gcggctgcat catccactct ctgcctttgt ctttccgtgg ctgccctccc tctgcatctg |
| 69601 | tctctgtgtc cactcctctt tttataagat cgccagtcct tgggtctagg ccttgggtct |
| 69661 | agggcccacc ctaatgcagc gtgacttcat cttaactaat tacatctgca aagaccctgt |
| 69721 | ctccaaataa ggtcatattt tgagcttctg agtggaagtg gatttggggg acagtcatca |
| 69781 | acccagcaca atgcccctcc tccccatgct ctctaaagag agaaggatct tggacatctt |
| 69841 | gattggacac gatgacacga agacaaaagt ttctgactag ctaggccaag ggtggacaca |
| 69901 | ttgtccctgg agtgacaagt cgggccagtg atcttgtagg gacaagccta catccagagc |
| 69961 | agtgctggta taggggcaag ccaacatccg tggcagtgct ggtatagggg caagccaaca |
| 70021 | tccatggcag tgctggtata ggggcaagcc aacatccggg gaaaaccaaa ggcctgtagg |
| 70081 | aaaagcctat ggggaggatg gagagaagag ggatggagcc ctggatgtaa cggggacctg |
| 70141 | catcctgtat ccctgtccct atgatgctgt gatggtgcct gactttctgt agaccaaagc |
| 70201 | tctccttttt tttttttttt ttgagacaga gttttgctct tgttgcccat gctggagtac |
| 70261 | aatggtgtga tctcagctca ctgcaacctc cgcctccggg tacaagcaat tcccctgtct |
| 70321 | cagcctccca agtagctcag ataacagtca tgcaccacca ctcctggcta atttttttgt |
| 70381 | atttaataga gacggagttt caccatgtta atcaggctgg tcaagaactt ttgacctcaa |
| 70441 | gtgatccaca tgccttggcc tcccaaagtg ctgggattac aggtgtgtgc caacacaccg |
| 70501 | ggccccaaag ccctcctttt actggagcta ttttgcacgg gatcaatagg cttctgcagt |
| 70561 | ggtgctggcc atcctagcat ccctggcttt ccttccctgc tgtggtctat atagtttggg |
| 70621 | tggagctgaa cccatagagt tgccggctgc acatgggacc agtgccctcc ctttggacgc |
| 70681 | tgcaatcagc ttgggagttt actcacagct gaatttgagc aacatgagga ggggagattc |
| 70741 | ataaactggt agaatgaacc atagagctga cagtcacctg gccactttga ggagaaggcc |
| 70801 | tgctggagaa cgaagacctg gaggaagctg aactcagaca gatggacaga cagacttggt |
| 70861 | gacgtcattg gaggcaccag gtccagctga accttgaact ctgtcatcac aggaaccagt |
| 70921 | aaattcacct ttaagcccaa gttgagtttc tgtcactcac ccccYaaaaa agtgctgatg |
| 70981 | ggagggtgtc tgggtaacaa ctgcatgcca gttgaccaca tccatgccgc tgccagcccc |
| 71041 | ggatctgggg tagagtctag ccgcctgagg gcatcaagtt tgtggcccat gagggtcgta |
| 71101 | gccatgctac agggcagagg acaagaaccg ggtccccatg agaccaaaag gagacaatag |
| 71161 | gtggaaagga tggcctgcac tgaagccggg gccaccaggc ttgtcgcccc tcactctgtg |
| 71221 | gacagactgg gttgcaccat ttctgaggga atctccatga ctcagtcctg ggcattgcgg |
| 71281 | ctggggccag tactcaaaat ggcagcagac gcagtcggcc tgccagcccc tgctagctct |
| 71341 | tgtttcaggt cagaggaaga aagtactggt ggcctctggg gatccagaaa ggactggtgg |
| 71401 | cctccaggtg tccaagagtc ttctcttcaa cgaacatggt gagggggagc attgtgctgg |
| 71461 | gttgatgagt gtcccccaaa ccaacttcca ctcagaagcc caaaatatga ccttgtttgg |
| 71521 | agacagggtc tttgcacacR taactagtta agatgaagcc atgctgcatt aggccagggc |
| 71581 | cctgtgtcca gggccttccc tgtgaggtgg ccgccaggag gccgacctca gggacacaag |
| 71641 | gactggaatg tgggggtctt tggagaggca tgacgccccc tctgtgagtg gacagacatc |
| 71701 | aagaatgaag ggagccctca acggggatgg agcagcccct gagacctgag ggtcccaccc |
| 71761 | tgggacaaag gtgccctgag ctcaagccag gacacccagc agctgccggc caggctgtgc |
| 71821 | ttcccccaca gcctctccac accctcgctg tgtcttctgt aagatgaggt cccctccatt |
| 71881 | accctgggcc agggatctca aaacggaagg ccgccaacca gtcagcggcc aggggctgga |
| 71941 | gcccatggtt tcccatggct cccactagga cacttgccag agagggggtg ggtgagctgg |
| 72001 | ttcccatgga gacaggctca ggatccccag aggccaccag tcctttcttc gtctgacctg |
| 72061 | aagcaagagc aaggccaggg gcttacaggc tgaccgtgtc tgcctccgtc tcaagggctt |
| 72121 | agactgagtg agactggcag aggccctcca ggtcactccc attgtttcac agatgaggaa |
| 72181 | accgaggcct accgggaggg agggccttcc gacaagtcac ggaactgatc gggggagagc |
| 72241 | tggatgcctg ctacaccgtg gctctgctgt ctcccgtgga aacccgggcc tctgttcgag |
| 72301 | cctgaagtgg cctctctgca ccaggctgct gggagacctg cagtcaccaa tggcaacgtc |
| 72361 | actcttgcaa cagccagtgg ggtgtggggc agcagacgaa gctcgagtac cgcctgggtt |
| 72421 | ccaagacaga atgagacaaa gtgaggggtc atatgggtgc ctggcctccc aggcagatgt |
| 72481 | ttctgttacc aggtggtctt tgaattctgt cctggactaa atatagttcc ttgggcattt |
| 72541 | ttgatcttgg taatcatgca accatggtac agaaataatt ggtccgctct catcttttcc |
| 72601 | agattacagt tcggggcagc tgagtaggcc ctgggtagag ggcacagagg tatggctcgg |
| 72661 | gtgcaggcgg gaccctgggt gacacccacg cagggtagag cccctccccc ggcctcggtc |
| 72721 | tccccacctg tgtaacgagg aagtggactg gacatgactc acggttcttc catgtccagc |
| 72781 | attctgcttt cagcatcacg ggaacgagag gtgatttccg taagcaggct ccaaggtgcc |
| 72841 | accaccaagc ccagaagtat ctttcccact ttgcagattt cctacagacc cagtattctc |
| 72901 | tcttccatgg gttgagtttc taaaatcaca tgactgtatt tttcttttaa tacagaaaag |
| 72961 | cataataaag gcataaaagc ccatgcccct ctgcagagca ccctgttgac attttctgaa |
| 73021 | agtaagtcat ctatgcacct gggaagaaag cacagaggac tcagaaaggc caaaaacaaa |
| 73081 | aacaaaaaag ccccacctca gcccctgccc ctctcccgga gaagccggag ttgtcagccc |
| 73141 | atgcagcaga gttggtcagg agctcaagtt cacatccccc aaccgccaca tactacctgt |
| 73201 | atcacctggg caggttctcc caatacctca gtgtcttcat ctgcaacgtg ggtaagatgg |
| 73261 | gaatgcccac ctcctgggga actcaggact cgctgagtta actgcattca gggacctgaa |
| 73321 | agagactcgc caggcagtaa ccccagtcca cttgttccat gctggtgtga gcagtgactg |
| 73381 | catattcttc caaaagcatc taccccatct attttttttt tttttttttg agacagggtc |
| 73441 | tccctctgtc acccaggctg gagtgcagtg gcatgatctc agctcactgc aacctccacc |
| 73501 | tcccgggttc aagcgattct cccgcctcag cctcctgagt agctgggatt acaggcgtgc |
| 73561 | accaccatgc ccagctaatt tttgtatttt tagtaaagac agggtttcac catattgctc |
| 73621 | aggctggtct ggaactccca acctcaggtg atctgcccac ctcagcctcc caaggtgctg |
| 73681 | ggattacagg cgtgagccac cgcgctgcct atactatcta tttgttaagc cccaaatgga |
| 73741 | tcatactttg gttcagcctc ttgcgttctt tgccttgttt attttagaga cctgtctatc |
| 73801 | tcaatccatg cagataaatg ccttgctctt tcttaggtgc caccaagcat agacacagac |
| 73861 | ctccccaatg ccccatcttt cgccattaat caacagtgtt gggatggtag gacaccagga |
| 73921 | gttagacacc tcaaagcacc ggttctggcc acactggtta tgcatattgt actcgactga |
| 73981 | ttcacactct catggccctg gatgagtgtc tctggccatc ccgcaggctg ggtgcttccc |
| 74041 | agtctgagcg tggtggcctg ttcttctcat ggctattacc ccgggcctct gtgggtccac |
| 74101 | cagccagagt tctgatgccc ggcgcgctca caggagcctg acttggggac agctttatga |
| 74161 | ggcagtgcag ggaccttgag gccatagtgg tgcagcagag aggtggggat gaggaggggc |
| 74221 | cgccagggcc cagggcagct cctggactca ctgggagagc cgaagtccac aggcagctcc |
| 74281 | tcacgccttg tcaggcctgc cctgcactca gctcccagat tttatagaag gaagtccctg |
| 74341 | cctttaggcc cagtcaaaaa aaattggact gggatattaa caaccaactt tccctggtgc |
| 74401 | tttcctgagt caacctctgc agccggggct ggctcagatg cctgccttgg ggccctctgg |
| 74461 | ggcagcgtgt ggagtcaggg ccatctgtga tctcctcagc aggttccatc ctgaccctgg |
| 74521 | agagccagct gacttgtgga aacagcagga tgccattctg agccaagccc agtgggtagg |
| 74581 | tacacagggc caacccaggt cacccatcaa gtgaccacag agcaatgaca cccatctgcc |
| 74641 | ccatactgtc actcgcttag agaaagttcc agaagagggg ctccaagcat gtgtcccRtg |
| 74701 | ataaagaggc ggcctctgcg agggttaatt tcagtgtcaa cttggYgggg ccacagcgcc |
| 74761 | cagatatttg accagacatc actctgggtg tttctgtgag gggcttgggg atgagactca |
| 74821 | cattggaatc agtgggccag gtaaagcagt ttcccctccc taatatgggt gggcctcatc |
| 74881 | caatcagttg acagcctgaa taggacaaag ggggtcccgt cccctgagta agatagaatt |
| 74941 | cctcttgcct gccagccttt gagctgaatg tctagctttt tcctgccttt ggacttgaac |
| 75001 | tgaaacgtcc gctctgtcta agctcgggct gctgtagact gtgtggctta agcatcaaat |
| 75061 | attcatttcc caccattcag gaggctggaa gtccagcacc acggtgctgc caatttgatt |
| 75121 | ttggtgaggg cccacttact gctggccttc ctctctctac gtcctaatgt gacagagatc |
| 75181 | agagagagag agctgccctg tctcttctta taagggcact Ratttcgttc attagcgctc |
| 75241 | caccctcctg acctaatcac cttcaaaaca ccccacctcc taagaccatc acattggagg |
| 75301 | ttggggtcca ccatatgaat tttgggggga catgtctagt ccctagtagc ctcttctggg |
| 75361 | tctccagctt gctgactgaa aatcttggga cttatcaatt cagttattcc aggagccagt |
| 75421 | tctttacaat aaatccctgt ttgtgtgtgt gtgtgtgtgc gtgtgtatgt gtgtgtgtgt |
| 75481 | gcatgtgtat gtgtgtgcat gtgtgtacat tctgttggtt ctgtttctct ggagaacctt |
| 75541 | gactaataac attctccaag gtgacaagga gactgacagg gggagagtga agtttaacac |
| 75601 | atcggttaaa atagtcacaa gattctcatc aactgccaca aatggcatct ggccacagcc |
| 75661 | actgagcttg gccacttctc cggctttgca aacgccgctg gatgcgcccg gcctgctgtc |
| 75721 | ccacagtccc accctgcctt ctcctacgtg tcctttctga actgccttcc aagctcctcc |
| 75781 | caaccccgta gggtccacct cattgtgcct cctttgtctc ctcctccgca gctcccctcc |
| 75841 | caacactggg ttcctagctc ctagaggtca gaggtcacac ttttccctct ctctgtctcg |
| 75901 | gtaggtgaaa gcagtaagtg gcacctaacg ggaagtgatg gggttggtgg ggaagagaac |
| 75961 | aaagtgtgga tcagtcctga ggaggctgtg caaagaccca gtgctgggtc caaggcccaa |
| 76021 | ccccaagaac caggatcttg tgcctcagtt tcctaactgt aaaaggggat gttggaatca |
| 76081 | cgtgtgtcaa agatcccttc caattctgtt ctttgtgggt tttctcatgt ctagagctgg |
| 76141 | aagagggcaa gcagagaacg cccccgcctc caggccccga gcactgtgag ctgtctgcct |
| 76201 | gcacagggct gcctgacccc accctcctga ccacgtcccc actcctgggg acggttcccg |
| 76261 | gcctccacgg catcccgggg aaggcaacag agatggactg gagggcacgg gaggacacgc |
| 76321 | attcactgac cgtccagccc agcaaagcaa gaaaccgtca aaaactaaaa attccatttg |
| 76381 | taaacaagat cgaattattt gattgtgaaa ccagggtcca tggaatttaa aataatttaa |
| 76441 | tatgaaacaa tatcatttga aatgctggcc agccaaggac ctggacgggg agggggactc |
| 76501 | tgtcctcctg tgcccagcca cgtggaaacc cagcctcaga gaataataac aaatcattct |
| 76561 | acagcaagtg tggctgagat tcagctacgt ggcagcccca ggccaggccc ggggggcagc |
| 76621 | cggcgaagac tcctcgtcct ctcagagttt acggtctaac tgagcagacc agggttggac |
| 76681 | aattaaataa cgatgccaga aagcaggaga ggagtgagtg ggtggcactc aagccaaaag |
| 76741 | ggaggcctga tagagccaag gggagtgtgc tgtgggccat ggcactgcca gaggaggctc |
| 76801 | ttgggtgctc tggcaccgca gtgagcagct gggggcttgg taagtaggga aagccagcac |
| 76861 | tcagaggaga aggtggcaga aaaagaaata ttcagaatag gaagcgtgat catttgcaac |
| 76921 | cccagctttg tcttgtactg cccatgccaa cttgggcaag cacgttatcc caagcctcag |
| 76981 | tcttctcatc tacagaatgg gaacaatccc agtccctact tcctggctgt tgtgagggtc |
| 77041 | ggaaatcatt cacgtgcagc cctctgggag cccgtgtccc tgacagacag tggtgctcca |
| 77101 | ggtggagcac gaggcaccgc tggcgctgta agcagatgcc tgtggaggga ggggaggagc |
| 77161 | aggtttccta gaggcctggg aggggctgat gcctctgcag tgggctctgc actcagctgc |
| 77221 | cttgaaatcc cagcaacagt gcctgcctgg agtgacggga gcaggctctt tggggaccgg |
| 77281 | ggggtcagga gggcacatgc acgtgccttg cagctggtgc ctcttggaag gatggggagg |
| 77341 | cggggacccg gcagaccctg gtgcaccgac actccacagc catgtcttct gcctcaacat |
| 77401 | aaacacgatt gggaatagtt caggactgta agagcaagaa aaacaaaccc agggaaacca |
| 77461 | aagagcgcgc aaatgctaac tttcatgtca catccagaaa ccacatgttc atcaagccac |
| 77521 | ctgcgggctt gtaactcagt ggggagacac agggttgttg tccttagaaa aatcaagcta |
| 77581 | tccttacagc tgcaagggga aaagtccagt tgctgcctcc cctccatcat cctggaccag |
| 77641 | gtccccccac agaacagccc tttgaggcag ccagggagct tcccgtcgct gtggccacct |
| 77701 | ggggcatggc ccctaaacct ttccgtgcac agggggttgg ggaaaagccc cggacctcca |
| 77761 | ggctgatgag tggcaggccc agaaccccca tcagggtgcc cagctcccca gtctccctgt |
| 77821 | ctcaccacca ccccaacctg ggctcagccg cccccgttca gggcaggcag gacttgtcct |
| 77881 | ggcctcgggg aggggccggg gaagaggaga gaagacccca gtggggcccc ccaggcccgc |
| 77941 | acatgccgcc cgactacctg tgggcttggg gagggctcca gatcactcct gccctcagag |
| 78001 | acggcgatgc tgagaagtgt tatttttatt cctccccaag ccttgccaga ttacattgtc |
| 78061 | aaggccagca ctttggagat atttccttgg tttcgcaatt cacacagtga ctaacacatg |
| 78121 | ttacattttg aaaacttctc tgggtaaaaa tttaagctga attgtaaata cataaaagtc |
| 78181 | tcaacgttaa tggggtacat gatatctcag tgactattgt gtaacaccaa gattcggaaa |
| 78241 | ggatgtggaa ttccatatag tcgggtgagg atgccgtccc agagccgccc tgtgaaagca |
| 78301 | tcattgtgag cagcaacctt gcctttctct gcggttatgt aagttacaaa accctttgcc |
| 78361 | tcttggcctc gtgaggcatc aggaccctca ggaggctgga actgcagagc tccgcccgcg |
| 78421 | atgatgaatc ccctccaggg ggcatgtgca cacgtgcccg gcacaccctg ccaggggctt |
| 78481 | catgcttgtg acctccttgg caccatgagg cactgggcta caggtgacaa aactgaggca |
| 78541 | catgcatgtt aagggacttg cctaaggaca tcccctgaca gaggcaagca ggactcaatc |
| 78601 | ccagatggaa tgctcactgc tatgcccatc accccctcag cccctggtgc tgggcagcta |
| 78661 | tgagccatgc tgtgccgtgt catgcacact gcaccccccc cccaatgatg gcacaggatg |
| 78721 | gcccccacca ggacaggcga tgcatgggcg gtcacctggg gtcactgtca gagcatctgg |
| 78781 | actacgaaat gaaggggatg ggccacttgg cagtgtccct acgcaagcca gagctacagg |
| 78841 | agcacctggg cataggtgtg ggaaggaggc agggagagaa ggaaggaagc agagaagcca |
| 78901 | agagtggttg gggagtggct gtgttgggag ccagtccccc cactgcccag gctcctgccc |
| 78961 | agccgagtgc ccagagccct gcccatctgc agcagccctg cccagccctc ccgagcctcc |
| 79021 | acctgcctgt tcattgcttt gctggatgac gcttatcagc ctctacttgg ccagttttct |
| 79081 | gtttcatttc cccgttcaac attgatctcc acacaggact gtagactcca tgcaggcagc |
| 79141 | acactgtctg ccgggctcct ggccgcatca ccagagcctg gccccaggac tgggcatggt |
| 79201 | gggcattgag tagttatttg ttgaatgtat gcacctccga aagaaagagg gagccaccat |
| 79261 | cctcgcctcg ctgctgtggc tcactgagtg gctttaccac ctaagatgcc tgctgccttt |
| 79321 | ctcctgctcc ttctgtgacc tcagacaggc agtgcagggg aggggtaggc agggcagctc |
| 79381 | tctcctcagc agagctcacc ttgggccact caggggccag gaagtggcag tgcaactcga |
| 79441 | gacgtttatg acacaatcaa ctccctgctg aatattgatt tttcttcaaa gctcagggcc |
| 79501 | tatctgtctg ggctcctccc agcgccaccc ccagccctgc ctggcgagcc tggagactcc |
| 79561 | agcagcaagg cttcatgctc ccagacgctg caggtccccc tactccgccc tggtcagggc |
| 79621 | cctccccacc gacatcctgg taacttagac attgcaagcc acagctgccg cagtgtgcca |
| 79681 | gaaaacgtcc ctccaaagac acaggattta tgaaggactg ttctctgctg tgtcaggaag |
| 79741 | ggtattcaca caggttggga agcatctcat gtgctccttt attgactgtg ctgggaaata |
| 79801 | ctgaagtggg caaatgccgt ccatctgagc tctcaggaca cgtaggggat aaaggggagt |
| 79861 | tgaggctgac caggaggcta tcacaggtca gtgtgataag cacaaaaata aaagtatgcc |
| 79921 | cagggcacca ggacggcagg atccgctcga agtggggtcg acagaggctt cccaaagaaa |
| 79981 | ggaagtttcc accatctctt gcgattttca tttggcatct ccagaatgcc tgttctatta |
| 80041 | gaaccaacta aggccttaat gggattactc agctttcagt cataacaagt ttgtctaggt |
| 80101 | catgtaggga ggtactttaa agtcattctt ccttctcttt tattatagca atgacttaca |
| 80161 | atgcttgaaa aaacattttc cttaattcaa ttaccaagtg agctaaatgc atttgctgga |
| 80221 | gataattaag aatctttaca aatataaaac aattatccca tcaaaataca gtgctggatg |
| 80281 | ctattattca cgctgggttt tcaatcagag ccccctctcc ccttttaaag caattccaaa |
| 80341 | gccccctgtg cctgcctttg ctggccgtcc ctgtgagccc aggccatttg tcctgccagc |
| 80401 | tctccagcac catggaaggg cgccctgtct gcccgacccc tggcccggag ccaccagagc |
| 80461 | tctcttcgct cacctctctg gtcctagtct tggcccagag caggtatcaa aaacaacgtg |
| 80521 | acacagacca gaggagccgg gggagacgtg acaactaaat gcaatgaagt cccctggcgt |
| 80581 | gggtcctgga acagagacag gacattaatg ggaaaactag cgaaatccaa ataaagtctg |
| 80641 | gaattgagtt aacaatggcc caatgtcagc ttctgagtgc tggcaaatgt accacggtaa |
| 80701 | tgtgcgcggt tcacaaaggg aagaccgggt ggggggcgaa cgggaactct ctgtactatc |
| 80761 | gtaacttttc tataaatcta aaattgttcc aaaataaaag tttataaaaa caaagcaaaa |
| 80821 | caacaaatgg gactgagtgg gcaaaagaga aggcagaatg tctccattgc tgaagtgagt |
| 80881 | attcctggtc ttcagaaacc cgcctggccc ccacacccac cccccacctc cagttgcagt |
| 80941 | cccgtctcag ttccctctga agacaagtct cagggaccag ctgcctgcgg ttgctgcctc |
| 81001 | cacttcaaca ctatctctcc ccacgctatt tgtcctgagc cacaaagtga aggtgaggtg |
| 81061 | tcaaagtgag cacctgcagg acggcaccct ggatgtttag cctaagaaag ctcccccgtc |
| 81121 | ctccttagag aagcccccac attgcacacc aaggagtgag gctgaggcag tgctgctgtc |
| 81181 | agtattctcc aaggccgcta actggacctc tggacaacct gctcttcaca gggccccctc |
| 81241 | agtccctcct gtcctggcca caggccacag cctccctcag acaggctgga atgaaggaac |
| 81301 | acactgagcc tggtctgcaa accctggcgg gggcttgtac tggacagcag tgctgatatg |
| 81361 | gggtgggctg agtatagcag aggcataact tccatccccc aggtctgggt acagacacag |
| 81421 | gcatgacttc catcccccag ggctgtgaaa aggagacgct gtccaaggca cctcatctga |
| 81481 | cggtgcttgc gatgaccgtg caggacatca gaggggccag aagcacccac ggccctgcgg |
| 81541 | tcggtcgtat ttacctcatc attgccatgc ctttgaggag ctttcctggg ccttctgagt |
| 81601 | gcctgagctt gggaagtaac cagtggctct atgagatttc ccccagaggg cactacgatt |
| 81661 | ttcatgggcc tctcccacca taaaaatacc tgtatttaaa aacgtgttta aggaatacaa |
| 81721 | atccttctac cataaagata ggcatgtgtc cttgcggcac tattcacaat agcaaagaca |
| 81781 | tggaatcaac cttgatgcac ctcagtggtg gactggataa agaaaatgtg gtacctatat |
| 81841 | accatggaat acgatgcagc cataaaacag aacatcagac atcaggtcct ttgcagcagc |
| 81901 | acggatggag ctggagggat ggagctggag gccgtcatcc tacgcgaatt aacgcaggca |
| 81961 | cagaaaatca aatactgcat gttctcactt ataaatgtga cctaaacact gagcacacat |
| 82021 | ggatacaaag gaggaaacag ggaacactgg gacatactct agggtggagg gaggggagag |
| 82081 | gctgaggatg gaaaaactac ctttcaggta ttattcggat gacctgggtg acaaaattat |
| 82141 | ctgtacacca aaccccatga catgcaattt acccatgtaa caaacctgca catgtacctt |
| 82201 | tgaacctaaa ataaaagctg gggggaaaat tgtagtaaat gtccctaagt atgaaataaa |
| 82261 | taaataaaat ttaaaaataa aaaaattaat aaatcttcaa atgcttccac atattttttt |
| 82321 | ctggtttaag caaagttaga gagatgttct tgggccccta aaggtatcag gttgagacag |
| 82381 | acatcctcgc cctgggcctg cggagcttaa tggacaggcc gggtccccgg gccacactta |
| 82441 | ccagttaagt gacctggggc aagttactta accaccccat cttcaatccc tcatctgcaa |
| 82501 | aacagggata atgatacctg ctctgcagag cttttatgag gattgcttaa gctaattggt |
| 82561 | gtaaaagcat taattccagc ctggccccca ggaggcgatc aataaagaag cattactctt |
| 82621 | gtgaaatact ctagccatca gagcaggcaa caatgtttct gtcttctaaa acgttcccaa |
| 82681 | gagagcctcg gctttactct acaaataaga cctcttgatt ttattctgcg agcttaaata |
| 82741 | tctttgactc tctcattacc tgaaactcaa ggtccttaaa taggtctgag gcccctaaac |
| 82801 | gttcctctaa gtgccctgta gagccatcac ttagtggctg agacacactc gctggctttc |
| 82861 | ccatcgacag atgcctcggt ggccctttct ggatcatcta atccctgctc caggccactg |
| 82921 | gctcaagttc cctcattgta ataagtgcaa gggagggtgt ttatgaagga aagcgggttt |
| 82981 | gactgaagtc caggctgaac tcggaatttc cctgtttgct ttctcttcct ccccgggcgt |
| 83041 | gctgatggac cagacctgcg ggagctaact caacaggcct ccagctgcca gagatttgag |
| 83101 | atgttagtca gggctcaggg gcggggggct ccgggagcag ggagcaggga gctggagccc |
| 83161 | tgaaaagacc tcagcgtctc cttgtcagga ctgaacttcc accttgggtc ctcgctgagt |
| 83221 | cttattttgc taatgagatc agacaggctt gtaattttag gtaactgcaa ttgctaagac |
| 83281 | tggaagagag actcattctc tggcattccc acaaattctt cccagtttgt ttagcttagg |
| 83341 | aaattaggca agagcaggta gaaaaacggc aggtcaaaca caagccattg caagaaactg |
| 83401 | actcatgctt taccaacagc gttattcatc ccccccgttc ctctccctgg ctatgtattt |
| 83461 | attcgtttat tcagcaaaca ttgattgagc acctaacatg tgtctctgcc ttttcacacc |
| 83521 | ctcaggccct tgcccaggct gcatcctccg ccatgtcctc ccgttgaccc actgtgtatc |
| 83581 | cttctagacc aggtcaaagg ctacctcctc tgggaagcct gccccaaagt tccatgccag |
| 83641 | acccaatccc tgcctctctg taggaaggag actaccatgg ctcccaagag gagcactggc |
| 83701 | tgccgagtca gaagaggttg gctgagaagc acggctccag gacatccctg atgtgtggct |
| 83761 | tgggcaaggt actgaggctt tctgctttct ttcctcttct ataaagtgtg aatactaata |
| 83821 | cctcccaaaa ctaagtgccc agggtgctac ataaaataca ctacacatca ctttccacgt |
| 83881 | ggaactgagc ttacaagaaa ggcaaggagt ttcccagagg ccaaaggcag gggaaagcca |
| 83941 | agagcgagag tgataagtcg gtgctggcat gaagccacct caggtgtcag gcagtcccag |
| 84001 | tggctttcag tggccatcca ggggcagaga gaaggtggga tggcaggatt gccatccttg |
| 84061 | caccaaacca gaacctcaga ggttctaacg ccagtggagg ggcaacttga gaaaaccacc |
| 84121 | tgctgcatgt ggaggcttct actgcttacc catattaaat gctcttcttc ctccaggcga |
| 84181 | cagatgaaca cagtctctga ccccagcaat tagcctgggc ttgggactag ctctggccaa |
| 84241 | agggctctga gaggaagcga tgtgaatcgc tcctagacta aagccattga caccaccaga |
| 84301 | ctcggcagcg ccatctctct cctgtggcac gtgtggaggc tgagcctgaa aatggtaacc |
| 84361 | tggacccagg ctgctccagg gaagacattt ccctggagag ttactcaagt ccccagcaaa |
| 84421 | ctttgcagga gccataaaag caagaaataa agcatctgtt gggtttagcc agtgagagag |
| 84481 | ttcctttgtt gctgcagcag aacttaggct atcctgacta attcagctag taaaagaagc |
| 84541 | agcaggagca cttgtctgtc tctctctggg atccaggtgg aggacaagtc tcccctgaca |
| 84601 | atcacagcca cagacctcac ttcacaggag ggtggggttc gaattcatac tccctttgtg |
| 84661 | ttcaaggaaa ctccaaacca taaaattaaa gatatttcag gtatgtagtg ttccgtgact |
| 84721 | agaagcaaac tcagatcccc caacctgacc tcacaggatt cccacaaata aagactcacc |
| 84781 | agaacaggca gtcgaaatcc caaattcaaa ccccaagaac ttcaaacaaa aattactcac |
| 84841 | tagggaattt ttaaaaataa ttttaatgca taaagaaatg aattgagagc caaaactatg |
| 84901 | ggcaattagc aaagtactat caaaagtgac taaataaatt tgaaagagaa ccaaacagaa |
| 84961 | ctatgaaaat cttattaacg atatgaaaac ctatttggta aaactgcaga ttcgaactgg |
| 85021 | ctcaacacag aattagtgaa atgaaaggta gaattgaagc aattacccag agaatcacag |
| 85081 | agagaaacgc tgctggagaa aaaaaaaaaa aagaggtgaa gaggcatcga ggcagagtga |
| 85141 | gtagcgtgat agctaattaa cgttctggaa gaggacagta gaaataacag ggctgggggt |
| 85201 | gcatgcctaa agctataatg gctaagaatg ctccaagatt gacagtagat gtgaatcttt |
| 85261 | agattcaaga atactagtga gtttatagca ggataaataa aaatatgcct tgagatatca |
| 85321 | tagtagaact gcaggatacc aaagacaaaa ggaaagcata tgcttattat aaattcatca |
| 85381 | gatttattaa tttgtaagag aaaaatgtat caagcacctg ctaggtgcca ggcagtgttg |
| 85441 | tagacacttg ggatatatcc gtccagaaag agacagaatt tgtgttcatt gtcaccagga |
| 85501 | agacaggtga caaaaaataa acataataaa taagtaaatt atacagtgtg ttagatattg |
| 85561 | gcaaatgcta tcaattggaa tatatggaaa aagaaacaaa aaaggatgag gagactctga |
| 85621 | gtatgtgtgt ggttgggaga ggctgagcca gggcaccata tttaataaaa tgtcagggga |
| 85681 | gttaacaaag ggaagagata acctacagga taggagaaaa tatttgcaaa ctctccatcc |
| 85741 | aacagggggt taataagcag aatgtataaa gaacgcaata gcaaataata acaatctgtt |
| 85801 | tttaaaatgg acaatagacc tgaatagaca tttctcaaag gaagacatac aaatggccaa |
| 85861 | cagggatttg aaaaaatggt caacatcact aatcatcagg gaaatgcaaa tcaaaaccac |
| 85921 | aatgagacag catctcaccc cagttaaaaa tggatattag caaaaagaca aaaagtaatg |
| 85981 | gatactggtg aggatgtgga gaagaacact gttggtggga atgtaaagta atacagtaat |
| 86041 | tagggaaaac agtatggagg ttcctcaaaa acctacaact agaactatca catgatctag |
| 86101 | aaatcccact gctgggtatg tatcctaaag aaaggaaatc aatatgtcaa agtggtatcc |
| 86161 | gcactcccat ctttattaca gcactattca taatagccaa gatgtgggat caacctgagt |
| 86221 | ccatcaacag atgaatggat aaagaaaatg tggtatatgt atgcaatgga atattattca |
| 86281 | gccataaaaa ggatgaaatc ttgtcatttg cggcaacgtg ggtggaactg gaagtcatta |
| 86341 | tgctaaatga aataagccag gcacagaaag ataaatatgg catcacatgt tctcagtcgt |
| 86401 | atgtgagagc taaaaaaagt ggatttcatg gggagtggaa tggtggttac tagagctagg |
| 86461 | aagggcagga gggagggaga gaagttggct aatgggtaca aaaatacagt tagatggaag |
| 86521 | gagtaagttc tagtattcaa tagtatagta cagagactat agctaacaat aagttattgt |
| 86581 | acatttcaag atagctaaaa aagaagaact ggaatgttcc caacacaaag aaaagataaa |
| 86641 | tatttgaggt gatagatatt ccaattactc tgatttgatc attacacatt gtatacagat |
| 86701 | atccaaatat cacaggtact cccaaagtac acacagctac tacatatcaa tttttaatat |
| 86761 | tacacctgaa aacaatttca agagaggctt cattgagaag gtaaaatttg ggaggggaat |
| 86821 | tgaaggagag gaaggagttg gtgagaccga gatatggaag agtgtctctc aggaagagaa |
| 86881 | gcagctaggg catcctggca atttgtgtat cttcatttac aaagtgtctg tccaaacctt |
| 86941 | ttgtccaact ctttaattgt tgcatatatt atattattga gttgtaagaa tttcaaatat |
| 87001 | attctggata taactctgta tcagatatat gtattgcaaa tattttctcc ctgtctgtaa |
| 87061 | cttgtctttt cgttttctta acaggtcttt tcaaatgtag acattgttta ttttgatcat |
| 87121 | gtccattttt atctattttt agttttatgc ttgtgggatt gttgtgttct gttaagaaat |
| 87181 | atttgtcttc ctcaatgtaa caaagtgtct agaagtttta cggttttggc tttgtttagg |
| 87241 | gctgtggtcc atcttaagtt aatttttctg cattgtgtga ggtagggttg aagtttatat |
| 87301 | ctttatggat gaatagctaa ttattccagc aacatttgtt taaaagtctt cccttttccc |
| 87361 | aataatttag cttggccctt ctttcaaaag tcaattgaaa aaatatatat catataaaga |
| 87421 | cttattatac tatatgtatt tttccagact ctttattctg ttccattgag ctgttcttac |
| 87481 | actaccacac taccttaatt attatagctt tatcatgagt tttaaaattg aatactgtaa |
| 87541 | gttcccctca ctttttttaa attgctttgg ctattctatg cccactgcat ttctatataa |
| 87601 | attttacaat cagcttgtcc atttctacaa aaaagacaga tgtgatttta gttgagatta |
| 87661 | agttgaatct atggatcact gagggctgaa tagacattta acaatattga ggcttcagat |
| 87721 | tcatgaactt ggtatatctc ttcgcttatt tatgccttct ttcatttccc tcagcgatgt |
| 87781 | tttgtagttt ttagtgtaca ggtcttgcac atatttgtga gatttatctc taggcatttc |
| 87841 | atacctttta tgctattgta aggggtattg tttttaaata tctttttatt tggaggtaac |
| 87901 | gttaaaatat cgaaagagtc acatccacag tacagagact ccctgtatat tattcaccca |
| 87961 | gcttgccctc gtgctaacat tctacataat cacaatctaa tcatgaaaac taagaaatta |
| 88021 | atattggtac aatactatta actcaactac agactttatc tggattctac tagttttcca |
| 88081 | ctcatgttat ttttcacttc caggatccca tccaggatcc tacattgtat ttagttggca |
| 88141 | tatcccctta gtcttctaca atctatgaca gttcttcagt attcctctct ttcatgactt |
| 88201 | gaagatgctt tcatggcttg atggtctgtc agatattttg tagagtgtct ctcagttggg |
| 88261 | tttggtttga tattttctca cgactattca tggtcacgct ttatcaagaa tactactgga |
| 88321 | gttttgtacc cttctaagtg catcaaacta ggcagttacc ttatttatta ctggtgatgt |
| 88381 | gagccttggt aacttggtaa agagagtatc tgtcctattc cttcactgta cagttactat |
| 88441 | ttttcttttt gtaattagta actcttggga aagatacttt gagactgtgc aaatgtcctg |
| 88501 | tttgttaaac ttttgcccac taattttagt atttggtgtt taacttgcct ccagcaatta |
| 88561 | ttgctgtgga atttcaatga tttttctatt ttcctcattt cttctacatt tgttaatagg |
| 88621 | aattcttctt taatgaggtg ttgttccttt tctcccattt atttattgat acttataatt |
| 88681 | taatattata tattgcacat tatttttata atgtaaaata taaaaattat atatttatta |
| 88741 | tgtatatgtg tgtgtgtata tatgtaaaat aacctgagga aaagatttta ttattttggt |
| 88801 | tataatccaa tattattact atttattttg ttgctcaaat tgttccagct ttggccattg |
| 88861 | ggagctcttt caaattggtt cctgtgtcct ttcagtaccc atgatccttt tggtttttga |
| 88921 | gtatatggtt tatcatatgg tattattttt tatttcaatc accaactgtt ctttgttagt |
| 88981 | atatagacat gcaatttatt tatgcatatt taaaatgagt ttcttacaga caacaggaaa |
| 89041 | gtgaaccttg tttttttatc cagtgtcaca atctctgcct tttattggag agtttagact |
| 89101 | tttacgttta atacaattaa tcagtatcat tggatttaag tttaccatct agctatttgt |
| 89161 | tttttattta tcctatctgc tctctgttcc tttttctctt ttttccttgc ttctttaagt |
| 89221 | ttaattgagt atgtttttag gattttattt cttcactagt atctaacaaa ctatctcttt |
| 89281 | gttctaattt ttagtgtttg ctccattatt taaaatatac atatttaatt tatcacaatc |
| 89341 | taccttcaat tgatattata ttatcacttc atgtatgatg tcatattcct gctacagtat |
| 89401 | atttcaatct taccccaccc atccttcatg tttttgttgt catagatatt actttttaat |
| 89461 | atatcgtaag cccaaagtac atggttgaca ttctgtttaa aacagtcaat tgtcttttaa |
| 89521 | agacattaaa aagcgagaaa aaagtctttt aatagatatt tttaaatgca tgtagcacaa |
| 89581 | taatgtaaac agttcatgta aaacaccaat ttataatatt ctatatatat atatatatat |
| 89641 | atatatatat atatatatat atatatatat atatatatat atagtttcct tcaggtaata |
| 89701 | ggttggaagg gtttagaata aaatgaacat gcatcacatc tgttatttta aaatatttaa |
| 89761 | tcaagttttt aaaatgttta tgaaccacca gaatgccact gccacaaata tcattaccaa |
| 89821 | taaccagtct tttttttttt tttttttttg tgagacagga tctcactctg tcacccaggc |
| 89881 | tggagtacag tggcatgatc atggctcatt gcagccttga ctttcgcaga ctcagatgat |
| 89941 | actcccacct caacctccta agtatctggg actacaggca ggcacaccac catgctctgc |
| 90001 | tattttttac tttattttat tttatgtatt tttgtagaga tggggttttg ccatgttgcc |
| 90061 | caggctagtc tggaacttcc gggttcaagc aatctgccca gctcagcctc ccaaagcgtt |
| 90121 | aggataacag gtgtgaacca ccacactgcc tacaaataac cattctttaa gtgggctttt |
| 90181 | atttgtttaa aaaacttttt tgtgtttatc tacataattg ccattctcac agtcttcatt |
| 90241 | ccttttcaga gagccgggtt tctatctggt attgttttct ttatgcttga agaacgttct |
| 90301 | ttaacatttc tggtagtaca agtctgatga cgtcttaaaa tgtttatttt gccttcattt |
| 90361 | ttaaaagata ttttcgtagg atacagagtt ctagattaac agcttctttc ttgcacttct |
| 90421 | tttgagatgt taccccattg acttctggct tacattgttt ccaaaaagaa tactgttttc |
| 90481 | attttcacca tgttcctgtg tatgttacac gtctcatttt tctctggctg cttctaggat |
| 90541 | tttctcctta acgccggttt cagaaaactg cttatgatgt ggcttgctgt ggttttcttt |
| 90601 | gtatttatcc tgcttggtct tcattgcact tcttggattt atagatttat ggttttcttc |
| 90661 | aaatttggaa atttttcatc cattatatct taaaacattt tttgtcctct ccttgctttc |
| 90721 | gaagacttca atttttccat accttagatg acttactatt gccccatagt cactgcgaat |
| 90781 | ctgtttattg ttttccagtc tcttttctct gtgtgctgtg tctcagatca cttatagaac |
| 90841 | tatgtcttca agttcactag tctttgaacc agctgtcaag cttatccagt gaaatttgca |
| 90901 | ttttagacat agctctacca gttccacata gtttcttttt atatcttcca tttattttct |
| 90961 | cactatggtt atcttttcat ttaagtactt gaacatagtt ataataattg tttttaacat |
| 91021 | tgttgcccgg catttccgtt gttcgtatca tcctttggtc tgtttctatt gacggcctta |
| 91081 | ttccctggct gtggttcaca ttttctggct ttttggcaca tggattttga tcagatgctg |
| 91141 | aatgttgtga atgtttcatt gttgagtgtc tgtaatttgc catcctcttt taaggagctt |
| 91201 | tggggtttgt cctgcttggc agtttagtta cttccagttc agttacacac tttctaggct |
| 91261 | cgtttttagc tttataggac aggtctagwg gatcctttgt tctagagata gtttgggccc |
| 91321 | actgctaaag tgtggctctt ctaaggtgtc taccgaatgc ccggtgatca gcaagaagga |
| 91381 | attctgaact gcaattgcaa ttgagaactt gaacgatccc atctccacat ggactttatg |
| 91441 | aacgaacagt tcaacgcagc tccagtgaat tactctttga ctgacctggt gagttttacc |
| 91501 | tgactcaggc tttttttttt tttttttttg gtattcagca aagattaaag aggacccctt |
| 91561 | tgctcctatt ctgtagctct tttttgcaaa gatctttcct ttttgtaagt atgacccccg |
| 91621 | gcttttggtg acccccctgg agaagcctca gtgctctgct tagcttccca cccccacccc |
| 91681 | gagtcccaca cagcagcatg gaaacctcca agcagaaagt caggcaaaga aagggctcct |
| 91741 | tcctatctcc cgcttatctc ctcctgggag cgcagttgtt tgtctctctt gcccaRtgta |
| 91801 | aagaggaagg tcagtgcagc ccctgttgct ccatcatcat ggttgggagc agacatctgt |
| 91861 | cccaccacat ggttttgagc tgacaattga aaagaaactg acaattgaaa agaaacactc |
| 91921 | tggcttctga actgagaaca gacaatagga gtgcaagggc agaagcaagg agacttccta |
| 91981 | gggggctatc gcatcacagc catccagaaa atgaatggtt gggttcaaaa tgaatggtaa |
| 92041 | cattttgaat gcaaagctta cagaaatccc tgatagattg gatgtaagga aacaagacac |
| 92101 | tttgggaggc cgaggcgggc ggatcacgag gtcaggagat cgagaccatc ctggctaaca |
| 92161 | tggtgaaacc ctatctccac taaaaacaca aaaaattagc caggcgtggt gggggacgct |
| 92221 | tgtagtccaa gctactcagg aggctgaggc aggagaatgg catgaacccg ggaggcggag |
| 92281 | cttgcagtga ggcgagatcg taccactgca ctccagcctg ggtgacagag cgagactcca |
| 92341 | taaaaaaaaa aaagaaagaa agaaagaaag agagagagag agagagagag agagagaaag |
| 92401 | aaagaaagaa agaaagaaag aaagaaagaa agaaagaaag aaagaaagaa agaaagaaaa |
| 92461 | gaaagaaaga aagaaggaag gaaggaaagg agaaaggaag gaaggaagga aggagaaaag |
| 92521 | aagaaaaaag tcaagatgat acaaggtttg ggtctgagca actagaataa tctcgttgcc |
| 92581 | atccattgag atgctgcagt ctgtgggtgg aacacgtctg gggctattgg ggaattgttt |
| 92641 | ggaggcatga gcttaaatgt ctacgaggca cacacacgtg gaaatgtcaa gctgggactt |
| 92701 | taaatataca attcctgagc ttgtgcagag tttagggttg gagatataaa tttggggtga |
| 92761 | gtgtaggata aagagaaaga acaaaatcta acactgagag gctggggcat ggaggaccag |
| 92821 | gagggctcaa aggagacaga aggagcagtc aaggaggagt caaggaggag tcaagggccg |
| 92881 | aggaccagga gggctttgag tcctgaagca gaacagtgca tgtcagaaag gaggatggac |
| 92941 | tgcagcagct ttgtaggtca agaagaacag ggcatagtag acaaccgctg ggtctgacag |
| 93001 | cgtggtggtc cctggtagaa tcatgtcgct aagagcctga ctttggggta aagaaaaagg |
| 93061 | aggaaaagga atgggagaca gagactacag acttactcct taatgaagct gtgctctaca |
| 93121 | aagaggagaa aaattgggac ggtagctagc atgggaatat ccaatgactg atccacgtgg |
| 93181 | atgcaaacgc ctgccccctg gcctcaattc aggacacctt ggcatggcca ccccaagtcc |
| 93241 | agggatctct gtgggctccg ccaagattct ggttgcagct gtactactgt tgaactcctc |
| 93301 | cggctgctca atcctgctcc tctctctccc cagcaagtac acacaactcc gagttgaacc |
| 93361 | ccacccaaga cacttccctt ccccgcaccc ccaaataaac cacttccatt ggaatcctgt |
| 93421 | ctcagaatct gtttctgggg aacccaaact tagcccaaca gcaggtctca aggctgacag |
| 93481 | gaatgtggga aattgaggga aagtgtggga accagaggaa gatgggctgt gggatgccac |
| 93541 | taagcaggtg ggggggcctg gatccagcgc acaaggcgag ggcctgcctt aggaaagggg |
| 93601 | cttgtcactt gcagcacttg gtgcaggatt caggtaggtg gcatctgcag aagatccctt |
| 93661 | ctgaataatt ctattttgtc agtgaaccag aagcaaggtt actactaagc atgaagactg |
| 93721 | gggagaagtg gggtttggag aagaaaggag actggtttgg gSgcgggggg ttgcagggaS |
| 93781 | gaaagtgaac agactagaca gtgagtcaca ggacaactgc gggaggacat cctcaaggtg |
| 93841 | cctaagaaaa caatagtcaa tctggacata tacctggtga aactgtattt caaggagaaa |
| 93901 | ggtaaaataa agatattttt agataaacac aaaaggagag actgtcccac caacaactct |
| 93961 | cacaaaagga cttttgaaat ggatgcattt caggaagaaa gaaagtgatc ccagacggaa |
| 94021 | gatctgggca ggaaaaagga gtgagaatct tacagaagga gacagggagt tgtggatcca |
| 94081 | tcaggcctga aatgtgatgg tcagttggtc agtttttgtg agtcaacata ttgctctttt |
| 94141 | ttctctctct tttttttttt tttttttttt ggattggtga ggtctggcta tgttgcccag |
| 94201 | gctggtcccg aacccctgag ttcaagcgat cctcccacct cKgcctccca aagtgctggg |
| 94261 | attacaggca tgggccacca cgcctggctc aacacattgc tctctaatgt tattgttgct |
| 94321 | tgccccaagt tccgtggagt cttctatcac ttgcagctga gcaccctgac ggatctacgc |
| 94381 | agccacttga gagcgcgctt tgccgcgtgc ttggcgagaa ggcgccaatc aaccctttaa |
| 94441 | tccaatcatg caatgattag ggccttccag gtggaggtgg agctgaggtc cttccgggag |
| 94501 | agagaaaaag cctcacagca ggcttactcc tataaggtga cacagacaca atctcgtcac |
| 94561 | aggatgagga gtagggaaat ctcccaactt gggcacacag cgtgtgatct cgccttgctt |
| 94621 | ttctccctgg aggtgcagca tcatcacagg tgttggatgg acagctggca accaatgtaa |
| 94681 | atgcctgttt ccccagctct aataaacggg cttaacagca catgccctcg tgcaccttag |
| 94741 | gaagccacat gtattgcagc tgggccactg ggaaaaggca cttctttctt tcagaataag |
| 94801 | ggccaactRa tgtatYatga atgacccaac aaccctacct cttaaaacgc tgtctgagag |
| 94861 | cctgagcaaa ctggacctcg ccagtggctg gtgagagctc tgtgttaatg gtgctggatt |
| 94921 | cctgcggggg cctgtgattc cccgctccgt agagagtagg acagcacccc gcctcctccg |
| 94981 | gacattgctg cacagtgggt gagccacctg ctgccctgaa caggaaggga aggaggagcc |
| 95041 | gtcggagctt |

Following are ICAM, MAPK10, KIAA0861, NUMA1, GALE, DPF3 and LOC145197 complementary DNA sequences (cDNAs; SEQ ID NO: 8, 9, 10, 11, 12, 13, 14, 15, 16 and 17, respectively). Following is an ICAM1 cDNA sequence (SEQ ID NO: 8).

NM_000201 [gi: 4557877] Homo sapiens intercellular adhesion molecule 1 (CD54),
human rhinovirus receptor (ICAM1), mRNA
gcgccccagtcgacgctgagctcctctgctactcagagttgcaacctcagcctcgctatggctcccagcagcccccg
gcccgcgctgcccgcactcctggtcctgctcggggctctgttcccaggacctggcaatgcccagacatctgtgtccc
cctcaaaagtcatcctgccccggggaggctccgtgctggtgacatgcagcacctcctgtgaccagcccaagttgttg
ggcatagagaccccgttgcctaaaaaggagttgctcctgcctgggaacaaccggaaggtgtatgaactgagcaatgt
gcaagaagatagccaaccaatgtgctattcaaactgccctgatgggcagtcaacagctaaaaccttcctcaccgtgt
actggactccagaacgggtggaactggcacccctcccctcttggcagccagtgggcaagaaccttaccctacgctgc
caggtggagggtggggcaccccgggccaacctcaccgtggtgctgctccgtggggagaaggagctgaaacgggagcc
agctgtgggggagcccgctgaggtcacgaccacggtgctggtgaggagagatcaccatggagccaatttctcgtgcc
gcactgaactggacctgcggccccaagggctggagctgtttgagaacacctcggccccctaccagctccagaccttt
gtcctgccagcgactcccccacaacttgtcagcccccgggtcctagaggtggacacgcaggggaccgtggtctgttc
cctggacgggctgttcccagtctcggaggcccaggtccacctggcactgggggaccagaggttgaaccccacagtca
cctatggcaacgactccttctcggccaaggcctcagtcagtgtgaccgcagaggacgagggcacccagcggctgacg
tgtgcagtaatactggggaaccagagccaggagacactgcagacagtgaccatctacagctttccggcgcccaacgt
gattctgacgaagccagaggtctcagaagggaccgaggtgacagtgaagtgtgaggcccaccctagagccaaggtga
cgctgaatggggttccagcccagccactgggcccgagggcccagctcctgctgaaggccaccccagaggacaacggg
cgcagcttctcctgctctgcaaccctggaggtggccggccagcttatacacaagaaccagacccgggagcttcgtgt
cctgtatggcccccgactggacgagagggattgtccgggaaactggacgtggccagaaaattcccagcagactccaa
tgtgccaggcttgggggaacccattgcccgagctcaagtgtctaaaggatggcactttcccactgcccatcggggaa
tcagtgactgtcactcgagatcttgagggcacctacctctgtcgggccaggagcactcaaggggaggtcacccgcga
ggtgaccgtgaatgtgctctccccccggtatgagattgtcatcatcactgtggtagcagccgcagtcataatgggca
ctgcaggcctcagcacgtacctctataaccgccagcggaagatcaagaaatacagactacaacaggcccaaaaaggg
acccccatgaaaccgaacacacaagccacgcctccctgaacctatcccgggacagggcctcttcctcggccttccca
tattggtggcagtggtgccacactgaacagagtggaagacatatgccatgcagctacacctaccggccctgggacgc
cggaggacagggcattgtcctcagtcagatacaacagcatttggggccatggtacctgcacacctaaaacactaggc
cacgcatctgatctgtagtcacatgactaagccaagaggaaggagcaagactcaagacatgattgatggatgttaaa
gtctagcctgatgagaggggaagtggtgggggagacatagccccaccatgaggacatacaactgggaaatactgaaa
cttgctgcctattgggtatgctgaggcccacagacttacagaagaagtggccctccatagacatgtgtagcatcaaa
acacaaaggcccacacttcctgacggatgccagcttgggcactgctgtctactgaccccaacccttgatgatatgta
tttattcatttgttattttaccagctatttattgagtgtcttttatgtaggctaaatgaacataggtctctggcctc
acggagctcccagtccatgtcacattcaaggtcaccaggtacagttgtacaggttgtacactgcaggagagtgcctg
gcaaaaagatcaaatggggctgggacttctcattggccaacctgcctttccccagaaggagtgatttttctatcggc
acaaaagcactatatggactggtaatggttcacaggttcagagattacccagtgaggccttattcctcccttccccc
caaaactgacacctttgttagccacctccccacccacatacatttctgccagtgttcacaatgacactcagcggtca
tgtctggacatgagtgcccagggaatatgcccaagctatgccttgtcctcttgtcctgtttgcatttcactgggagc
ttgcactattgcagctccagtttcctgcagtgatcagggtcctgcaagcagtggggaagggggccaaggtattggag
gactccctcccagctttggaagggtcatccgcgtgtgtgtgtgtgtgtatgtgtagacaagctctcgctctgtcacc
caggctggagtgcagtggtgcaatcatggttcactgcagtcttgaccttttgggctcaagtgatcctcccacctcag
cctcctgagtagctgggaccataggctcacaacaccacacctggcaaatttgattttttttttttttttcagagacg
gggtctcgcaacattgcccagacttcctttgtgttagttaataaagctttctcaactgcc

Following is an ICAM4 cDNA sequence (SEQ ID NO: 9).

NM_001544 [gi: 12545400] Homo sapiens intercellular adhesion molecule 4,
Landsteiner-Wiener blood group (ICAM4), transcript variant 1, mRNA.
ctttttgccatggggtctctgttccctctgtcgctgctgttttttttggcggccgcctacccgggagttgggagcgc
gctgggacgccggactaagcgggcgcaaagccccaagggtagccctctcgcgccctccgggacctcagtgcccttct
gggtgcgcatgagcccggagttcgtggctgtgcagccggggaagtcagtgcagctcaattgcagcaacagctgtccc
cagccgcagaattccagcctccgcaccccgctgcggcaaggcaagacgctcagagggccgggttgggtgtcttacca
gctgctcgacgtgagggcctggagctccctcgcgcactgcctcgtgacctgcgcaggaaaaacacgctgggccacct
ccaggatcaccgcctacaaaccgccccacagcgtgattttggagcctccggtcttaaagggcaggaaatacactttg
cgctgccacgtgacgcaggtgttcccggtgggctacttggtggtgaccctgaggcatggaagccgggtcatctattc
cgaaagcctggagcgcttcaccggcctggatctggccaacgtgaccttgacctacgagtttgctgctggaccccgcg
acttctggcagcccgtgatctgccacgcgcgcctcaatctcgacggcctggtggtccgcaacagctcggcacccatt
acactgatgctcgcttggagccccgcgcccacagctttggcctccggttccatcgctgcccttgtagggatcctcct
cactgtgggcgctgcgtacctatgcaagtgcctagctatgaagtcccaggcgtaaagggggatgttctatgccggct
gagcgagaaaaagaggaatatgaaacaatctggggaaatggccatacatggtggctgacgcctgtaatcccagcact
ttgggaggccgaggcaggagaatcgcttgagcccaggagttcgagaccagcctggacaacatagtgagaccccgtct
atgcaaaaaatacacaaattagcctggtgtggtggcccgcacctgtggtcccagctacccgggaggctgagttggga
ggatcctttgagccctgaaagtcgaggttgcagtgagccttgatcgtgccactgcactccagcctgggggacagagc
acgaccctgtctccaaaaataaaataaaaataaaaataaatattggcgggggaaccctctggaatcaataaaggctt
ccttaaccagc

Following is an ICAM5 cDNA sequence (SEQ ID NO: 10).

NM_003259 [gi: 12545403] Homo sapiens intercellular adhesion molecule 5,
telencephalin (ICAM5), mRNA
ccgtcctctagcccagctcctcggctcgcgctctcctcgcctcctgtgctttccccgccgcggcgatgccagggcct
tcgccagggctgcgccgggcgctactcggcctctgggctgctctgggcctggggctcttcggcctctcagcggtctc
gcaggagcccttctgggcggacctgcagcctcgcgtggcgttcgtggagcgcgggggctcgctgtggctgaattgca
gcaccaactgccctcggccggagcgcggtggcctggagacctcgctgcgccgaaacgggacccagaggggtttgcgt
tggttggcgcggcagctggtggacattcgcgagccggagactcagcccgtctgcttcttccgctgcgcgcggcgcac
actacaggcgcgtgggctcattcgcactttccagcgaccagatcgcgtagagctgatgccgctgcctccctggcagc
cggtgggcgagaacttcaccctgagctgtagggtccccggcgccgggccccgtgcgagcctcacgctgaccctgctg
cggggcgcccaggagctgatccgccgcagcttcgccggtgaaccaccccgagcgcggggcgcggtgctcacagccac
ggtactggctcggagggaggaccatggagccaatttctcgtgtcgcgccgagctggacctgcggccgcacggactgg
gactgtttgaaaacagctcggcccccagagagctccgaaccttctccctgtctccggatgccccgcgcctcgctgct
ccccggctcttggaagttggctcggaaaggcccgtgagctgcactctggacggactgtttccagcctcagaggccag
ggtctacctcgcactgggggaccagaatctgagtcctgatgtcaccctcgaaggggacgcattcgtggccactgcca
cagccacagctagcgcagagcaggagggtgccaggcagctgatctgcaacgtcaccctggggggcgaaaaccgggag
acccgggagaacgtgaccatctacagcttcccggcaccactcctgaccctgagcgaacccagcgtctccgaggggca
gatggtgacagtaacctgcgcagctgggacccaagctctggtcacactggagggagttccagccgcggtcccggggc
agcccgcccagcttcagctaaatgccaccgagaacgacgacagacgcagcttcttctgcgacgccaccctcgatgtg
gacggggagaccctgatcaagaacaggagcgcagagcttcgtgtcctatacgctccccggctagacgattcggactg
ccccaggagttggacgtggcccgagggcccagagcagacgctgcgctgcgaggcccgcgggaacccagaaccctcag
tgcactgtgcgcgctccgacggcggggccgtgctggctctgggcctgctgggtccagtcactcgggcgctctcaggc
acttaccgctgcaaggcggccaatgatcaaggcgaggcggtcaaggacgtaacgctaacggtggagtacgcaccagc
gctggacagcgtgggctgcccagaacgcattacttggctggagggaacagaagcctcgctgagctgtgtggcgcacg
gggtaccgccgcctgatgtgatctgcgtgcgctctggagaactcggggccgtcatcgaggggctgttgcgtgtggcc
cgggagcatgcgggcacttaccgctgcgaagccaccaaccctcggggctctgcggccaaaaatgtggccgtcacggt
ggaatatggccccaggtttgaggagccgagctgccccagcaattggacatgggtggaaggatctgggcgcctgtttt
cctgtgaggtcgatgggaagccacagccaagcgtgaagtgcgtgggctccgggggcgccactgagggggtgctgctg
ccgctggcacccccagaccctagtcccagagctcccagaatccctagagtcctggcacccggtatctacgtctgcaa
cgccaccaaccgccacggctccgtggccaaaacagtcgtcgtgagcgcggagtcgccaccggagatggatgaatcta
cctgcccaagtcaccagacgtggctggaaggggctgaggcttccgcgctggcctgcgccgcccggggtcgcccttcc
ccaggagtgcgctgctctcgggaaggcatcccatggcctgagcagcagcgcgtgtcccgagaggacgcgggcactta
ccactgtgtggccaccaatgcgcatggcacggactcccggaccgtcactgtgggcgtggaataccggccagtggtgg
ccgaacttgctgcctcgccccctggaggcgtgcgcccaggaggaaacttcacgttgacctgccgcgcggaggcctgg
cctccagcccagatcagctggcgcgcgcccccgggggccctcaacatcggcctgtcgagcaacaacagcacactgag
cgtggcaggcgccatgggaagccacggcggcgagtacgagtgcgcacgcaccaacgcgcacgggcgccacgcgcggc
gcatcacggtgcgcgtggccggtccgtggctatgggtcgccgtgggcggcgcggcggggggcgcggcgctgctggcc
gcgggggccggcctggccttctacgtgcagtccaccgcctgcaagaagggcgagtacaacgtgcaggaggccgagag
ctcaggcgaggccgtgtgtctcaacggagcgggcggcggcgctggcggggcggcaggcgcggagggcggacccgagg
cggcggggggcgcggccgagtcgccggcggagggcgaggtcttcgccatacagctgacatcggcgtgagccgctccc
ctctccccgcgggccgggggacgccccccagactcacacgggggcttatttattgctttatttatttacttattcat
ttatttatgtattcaactccaagggcgtcacccccattttctacccatcccctcaataaagtttttataaagga

Following is a MAPK10 cDNA sequence (SEQ ID NO: 11).

NM_003259 [gi: 12545403] Homo sapiens mitogen-activated protein
kinase 10 (MAPK10), transcript variant 1, mRNA.
gagaaatggcgtggcaggggacccagcgagcccagagggattttgccgctgcttcctctacccctgtatttcacgca
gctctctaaattgactcagctccaggctagtgtgagaaacaccaacagcaggcccatctcagatcttcactatggca
acttatgcaagaaactgttgaattagacccgtttcctatagatgagaaaccatacaagctgtggtatttatgagcct
ccatttcttatactactgcagtgaaccaacattggatgtgaaaattgccttttgtcagggattcgataaacaagtgg
atgtgtcatatattgccaaacattacaacatgagcaaaagcaaagttgacaaccagttctacagtgtggaagtggga
gactcaaccttcacagttctcaagcgctaccagaatctaaagcctattggctctggggctcagggcatagtttgtgc
cgcgtatgatgctgtccttgacagaaatgtggccattaagaagctcagcagaccctttcagaaccaaacacatgcca
agagagcgtaccgggagctggtcctcatgaagtgtgtgaaccataaaaacattattagtttattaaatgtcttcaca
ccccagaaaacgctggaggagttccaagatgtttacttagtaatggaactgatggatgccaacttatgtcaagtgat
tcagatggaattagaccatgagcgaatgtcttacctgctgtaccaaatgttgtgtggcattaagcacctccattctg
ctggaattattcacagggatttaaaaccaagtaacattgtagtcaagtctgattgcacattgaaaatcctggacttt
ggactggccaggacagcaggcacaagcttcatgatgactccatatgtggtgacacgttattacagagcccctgaggt
catcctggggatgggctacaaggagaacgtggatatatggtctgtgggatgcattatgggagaaatggttcgccaca
aaatcctctttccaggaagggactatattgaccagtggaataaggtaattgaacaactaggaacaccatgtccagaa
ttcatgaagaaattgcaacccacagtaagaaactatgtggagaatcggcccaagtatgcgggactcaccttccccaa
actcttcccagattccctcttcccagcggactccgagcacaataaactcaaagccagccaagccagggacttgttgt
caaagatgctagtgattgacccagcaaaaagaatatcagtggacgacgccttacagcatccctacatcaacgtctgg
tatgacccagccgaagtggaggcgcctccacctcagatatatgacaagcagttggatgaaagagaacacacaattga
agaatggaaagaacttatctacaaggaagtaatgaattcagaagaaaagactaaaaatggtgtagtaaaaggacagc
cttctccttcagcacaggtgcagcagtgaacagcagtgagagtctccctccatcctcgtctgtcaatgacatctcct
ccatgtccaccgaccagaccctggcatctgacactgacagcagcctggaagcctcggcaggacccctgggttgttgc
aggtgactagccgcctgcctgcgaaacccagcgttcttcaggagatgatgtgatggaacacacacacacgcagacac
acacacacacacaaatgcagacacacaacatcaagaaaacagcaagggagagaatccaagcctaaaattaaataaat
ctttcagcctgcttcttccccagggttctgtattgcagctaagctcaaatgtatatttaacttctagttgctcttgc
tttggtcttcttccaatgatgcttactacagaaagcaaatcagacacaattagagaagccttttccataaagtgtaa
ttttaatggctgcaaaaccggcaacctgtaactgcccttttaaatggcatgacaaggtgtgcagtggccccatccag
catgtgtgtgtctctatcttgcatctacctgctccttggcctagtcagatggatgtagatacagatccgcatgtgtc
tgtattcatacagcactacttacttagagatgctactgtcagtgtcctcagggctctaccaagacataatgcactgg
ggtaccacatggtccatttcatgtgatctattactctgacataaacccatctgtaatatattgccagtatataagct
gtttagtttgttaattgattaaactgtatgtcttataagaaaacatgtaaagggggaatatatggggggagtgagct
ctctcagacccttgaagatgtagcttccaaatttgaatggattaaatggcacctgtatacca
Other isoforms are isoform 2 (NM_138982); isoform 3 (NM_138980); isoform 4
(NM_138981)

Following is a first KIAA0861 cDNA sequence (SEQ ID NO: 12).

KIAA0861 Coding Sequence (cDNA) Corresponding to SEQ ID NO: 2. (cDNA from
positions 94 . . . 3189)
cgctgaattctaggggaggatggcgcccccatcatcacgttcccagagttttcggggttcaaacacatcccagatga
agacttcctgaatgtcatgacctacctgactagcatccccagtgtggaggctgccagcattggattcattgttgtta
tcgacagacgaagagacaagtggagctccgtaaaggcatccttgacacgaatagctgtggcatttccaggaaactta
cagctcatattcatccttcgtccatctcgctttatccagaggacattcactgacattggcattaaatactatcgaaa
tgagtttaaaacgaaagtgccgatcatcatggtaaactctgtctctgaccttcacggctacatcgacaaaagccaac
tgacccgggaattaggggggactttggaatatcgccacggtcagtgggtaaatcaccgcactgccatcgaaaacttt
gccttgaccttgaagaccactgcccagatgctgcagacgtttgggtcctgcctggccacagcagagctgcccagaag
catgctatccacggaagaccttctcatgtcccacacaaggcagcgggacaagctgcaggatgagctgaaattacttg
gaaagcaggggaccacattgctgtcatgcatccaagaaccagcaaccaaatgtcccaacagcaaactcaatctcaac
caacttgagaatgtaactaccatggaaaggttattagttcaactggatgaaacagaaaaagcctttagtcacttttg
gtctgagcatcatctgaagcttaaccagtgcctacaactacagcattttgagcacgatttttgtaaggctaagcttg
ccctggataatttgctggaagagcaagcagagtttacaggcattggagacagcgtgatgcacgtggagcagattctt
aaggaacacaaaaaactggaggaaaaaagccaggagtccctggaaaaggcccagctgctggcactggttggggacca
gctcatccaaagccaccattatgcagcagatgccatcaggccccggtgtgtggagctcaggcacctctgtgacgatt
tcatcaatggaaacaagaaaaaatgggacattttaggaaagtccttagagtttcatagacagctggacaaggtcagc
caatggtgtgaggcaggaatctacctcttggcttcccaagctgtagacaagtgccagtctcgagaaggggttgatat
cgccttgaacgacattgcgacattcctgggcacagtcaaggagtacccgttgctcagccccaaggagttttacaacg
agtttgagttgctgctcaccctcgatgcaaaggccaaagcccagaaagttttgcagaggctggatgatgtccaggaa
atatttcacaagaggcaagtgagtctgatgaaactggcagccaaacagactcgtccagtgcaacctgtggccccaca
tcctgagtcttcaccaaaatgggtgtcatcaaaaaccagccagccctccacctcggtccctctagctcgtcctctga
gaacgtctgaggaaccttatacggagacagagttgaactcccggggaaaggaagatgatgagactaaatttgaagtc
aagagtgaagaaatctttgaaagccatcatgaaagggggaaccctgagctggagcagcaggccaggctcggagacct
ttccccccgcaggcgcattatacgtgacttgcttgagactgaagagatttacataaaagagattaaaagcataattg
atggatatatcactccaatggattttatttggctaaagcatctaattccagatgttcttcagaataacaaggacttt
ctctttgggaatattagagaactttacgaatttcacaacaggacttttctaaaagagttggaaaagtgtgctgagaa
ccctgaacttctggcacattgctttctcaagagaaaagaagatcttcagatatattttaaataccataagaatctgc
cccgagctagggcaatctggcaagagtgtcaagactgcgcctactttggggtatgccagcgccaactggatcacaat
ctccctctttttaagtatctcaaaggaccaagccagagacttataaaataccagatgctgttgaagggtctgctgga
tttcgagtctcctgaagatatggagatagacccaggtgaactaggaggctcggctaaggatgggccaaagagaacca
aagattcagcattctcaactgaactacaacaagctttggcagtgatagaggatttgatcaagtcctgtgagttggct
gtggacctagcagcagtgactgaatgtccggacgatattggaaaactaggcaagctgttgctgcacggccctttcag
cgtctggacaattcacaaggatcgttataaaatgaaggatttgattcgatttaaacccagccagaggcaaatctacc
tatttgaaaggggaatagtgttctgtaagatacgaatggagcctggggaccagggattatctcctcattacagcttc
aagaaggccatgaagctgatgacactttcaattcgccagcttggaagggggagccatagaaagtttgagattgccag
tcgaaatggacttgagaaatacatcctgcaggcagcttcaaaagaaatcagagactgttggttttcagaaataagta
aattattgatggaacaacaaaataatatcaaagaccaaggaaatccacagtttgaaatgagcacgagcaaaggcagt
ggagcaggatccggaccatggattaaaaatatggaaagagctaccactagcaaggaagacccggcctccagcacagg
agggattaaaggctgctccagcagggagtttagctccatggacacctttgaagactgtgaaggcgcagaagacatgg
aaaaggagagcagtgctctgagtctcgcgggccttttccagtcggacgacagtcacgaaacctgttcctccaaatct
gctttcctggagaggggagaaagcagccagggagaaaaagaagaacgcgatgaggaggaaacggcgacccgcagcac
cgaggaggagcgcgctggggcgtccacgggccggctggctcctgcgggggcgacggctggtttccaggcgagggcgc
tgcgcccgaggacctccgcccaggagagctga

Following is a second KIAA0861 cDNA sequence (SEQ ID NO: 13).

ttgggcggagatgcctttaaaaaatcatccaccgcagcggtagaaacagttttgtttggctttatttatacggaatg
gtttttcagtgaaatgctgtcttgcttaaaagaagagatgcctccccaggagctcacccggcgactggccacagtga
tcactcatgtcgatgaaattatgcagcaggaagtcagacccctgatggcggtggagataatagaacaacttcacaga
caatttgccattctttcaggaggccgaggggaggatggcgcccccatcatcacgttcccagagttttcggggttcaa
acacatcccagatgaagacttcctgaatgtcatgacctacctgactagcatccccagtgtggaggctgccagcattg
gattcattgttgttatcgacagacgaagagacaagtggagctccgtaaaggcatccttgacacgaatagctgtggca
tttccaggaaacttacagctcatattcatccttcgtccatctcgctttatccagaggacattcactgacattggcat
taaatactatcgaaatgagtttaaaacgaaagtgccgatcatcatggtaaactctgtctctgaccttcacggctaca
tcgacaaaagccaactgacccgggaattaggggggactttggaatatcgccacggtcagtgggtaaatcaccgcact
gccatcgaaaactttgccttgaccttgaagaccactgcccagatgctgcagacgtttgggtcctgcctggccacagc
agagctgcccagaagcatgctatccacggaagaccttctcatgtcccacacaaggcagcgggacaagctgcaggatg
agctgaaattacttggaaagcaggggaccacattgctgtcatgcatccaagaaccagcaaccaaatgtcccaacagc
aaactcaatctcaaccaacttgagaatgtaactaccatggaaaggttattagttcaactggatgaaacagaaaaagc
ctttagtcacttttggtctgagcatcatctgaagcttaaccagtgcctacaactacagcattttgagcacgattttt
gtaaggctaagcttgccctggataatttgctggaagagcaagcagagtttacaggcattggagacagcgtgatgcac
gtggagcagattcttaaggaacacaaaaaactggaggaaaaaagccaggagcccctggaaaaggcccagctgctggc
actggttggggaccagctcatccaaagccaccattatgcagcagatgccatcaggccccggtgtgtggagctcaggc
acctctgtgacgatttcatcaatggaaacaagaaaaaatgggacattttaggaaagtccttagagtttcatagacag
ctggacaaggtcagccaatggtgtgaggcaggaatctacctcttggcttcccaagctgtagacaagtgccagtctcg
agaaggggttgatatcgccttgaacgacattgcgacattcctgggcacagtcaaggagtacccgttgctcagcccca
aggagttttacaacgagtttgagttgctgctcaccctcgatgcaaaggccaaagcccagaaagttttgcagaggctg
gatgatgtccaggaaatatttcacaagaggcaagtgagtctgatgaaactggcagccaaacagactcgtccagtgca
acctgtggccccacatcctgagtcttcaccaaaatgggtgtcatcaaaaaccagccagccctccacctcggtccctc
tagctcgtcctctgagaacgtctgaggaaccttatacggagacagagttgaactcccggggaaaggaagatgatgag
actaaatttgaagtcaagagtgaagaaatctttgaaagccatcatgaaagggggaaccctgagctggagcagcaggc
caggctcggagacctttccccccgcaggcgcattatacgtgacttgcttgagactgaagagatttacataaaagaga
ttaaaagcataattgatggatatatcactccaatggattttatttggctaaagcatctaattccagatgttcttcag
aataacaaggactttctctttgggaatattagagaactttacgaatttcacaacaggacttttctaaaagagttgga
aaagtgtgctgagaaccctgaacttctggcacattgctttctcaagagaaaagaagatcttcagatatattttaaat
accataagaatctgccccgagctagggcaatctggcaagagtgtcaagactgcgcctactttggggtatgccagcgc
caactggatcacaatctccctctttttaagtatctcaaaggaccaagccagagacttataaaataccagatgctgtt
gaagggtctgctggatttcgagtctcctgaagatatggagatagacccaggtgaactaggaggctcggctaaggatg
ggccaaagagaaccaaagattcagcattctcaactgaactacaacaagctttggcagtgatagaggatttgatcaag
tcctgtgagttggctgtggacctagcagcagtgactgaatgtccggacgatattggaaaactaggcaagctgttgct
gcacggccctttcagcgtctggacaattcacaaggatcgttataaaatgaaggatttgattcgatttaaacccagcc
agaggcaaatctacctatttgaaaggggaatagtgttctgtaagatacgaatggagcctggggaccagggattatct
cctcattacagcttcaagaagaccatgaagctgatgacactttcaattcgccagcttggaagggggagccatagaaa
gtttgagattgccagtcgaaatggacttgagaaatacatcctgcaggcagcttcaaaagaaatcagagactgttggt
tttcagaaataagtaaattattgatggaacaacaaaataatatcaaagaccaaggaaatccacagtttgaaatgagc
acgagcaaaggcagtggagcaggatccggaccatggattaaaaatatggaaagagctaccactagcaaggaagaccc
ggcctccagcacaggagggattaaaggctgctccagcagggagtttagctccatggacacctttgaagactgtgaag
gcgcagaagacatggaaaaggagagcagtgctctgagtctcgcgggccttttccagtcggacgacagtcacgaaacc
tgttcctccaaatctgctttcctggagaggggagaaagcagccagggagaaaaagaagaacgcgatgaggaggaaac
ggcgacccgcagcaccgaggaggagcgcgctggggcgtccacgggccggctggctcctgcgggggcgacggctggtt
tccaggcgagggcgctgcgcccgaggacctccgcccaggagagctgacctccctgcggacgcccccgctcctcggct
ccagagcgcccgcattcccgggagaggcggtgtgggggcccgggccctgcccagctacgcagaaagcagccggagcc
tcggcggcggcagaaaggggacaaccagggcctcctccgaggagcccgaggggtgtcctgggtgcgcgcctagctcc
gcacgggggacctcggagctgctctaaggcgcctgcagaggcgagcagagcccgcagcccacgccttctcgaccgcg
cacttcgacattcggagccgggcaattctttgctgcgtgggcttctctgtgctcccacggtaggatgtttagtagca
cccctggcctctacccactaggtgccaggaatgcgccaccccatcctccaccccgcccccaaatcgtgacaatcaaa
aatgcctgcagacacgcccgcatttctccagggcggggtgggaatcggttgagagccgcttagcccgagccttggag
gagccggagccgctcaaacccggcgggggccgcagactgggagctcccggtccgcctcccagcatccctgcgagcgt
tcatggggtgttcgtgttagtgccaagattgcttcgttgtagagagagttcgttccaagttactttctgaggtattt
tatgtatcgatttattagtttttaaatgagttttgttagtttcagttgtattttatttttagttttattagttgatt
ttcatttctttgtagctttctggttatttaattttatagtttcagttactggtttatagttactttattttcaaagt
tatttagttgttcattttcagttatttatatgtagttgttttgttttaggagttacagatgttcaaattaatttgct
tggaatttatttatttatttatttatttatttttcgagacggagtctcgctctgtcgcccaggctggagtgcagtgg
tgtgatctcggctcactgcaaaccgcctcccgggttcacgccattctcctgcctcagcctcctgagtagctgggact
acaggcgcccgccaccacgcccggctaattttttatttggatttttagtagagacggggtttccccgtgttagccag
gatggtctcgatctcctgacctcgtgatccccctgcctcggcctcccaaagtgctgggattacaggcgtgagccacc
gcgcccagccggaatttatttttaattatctttacaattattatctgagttatttaccttcatagttatttcacttt
atcttattggagtttttgagttatttatttttacagtaacttatttgactattaaacactcactggaagttcatg
(4772 bp)

Following is a NUMA1 cDNA sequence (SEQ ID NO: 14).

NM_006185 [gi: 5453819] Homo sapiens nuclear mitotic apparatus protein 1
(NUMA1), mRNA.
gcccacgaagaggtacgattccggagaatcgcgaggcagagcgggagcgcgcagccaggtggaaactaattctaagc
cagactgctggagatcaccctgttctagtgtgtggaggcttccaccaggagtctggagtgcaatggcacgatctcgg
ctcactgcaacctccacctcccaggttcaagcgattctcctgcctcagcctcccaagtagctgggattacaggcgca
ttggagtgactgtctggcatcaccaagatgacactccacgccacccggggggctgcactcctctcttgggtgaacag
tctacacgtggctgaccctgtggaggctgtgctgcagctccaggactgcagcatcttcatcaagatcattgacagaa
tccatggcactgaagagggacagcaaatcttgaagcagccggtgtcagagagactggactttgtgtgcagttttctg
cagaaaaatcgaaaacatccctcttccccagaatgcctggtatctgcacagaaggtgctagagggatcagagctgga
actggcgaagatgaccatgctgctcttataccactctaccatgagctccaaaagtcccagggactgggaacagtttg
aatataaaattcaggctgagttggctgtcattcttaaatttgtgctggaccatgaggacgggctaaaccttaatgag
gacctagagaacttcctacagaaagctcctgtgccttctacctgttctagcacattccctgaagagctctccccacc
tagccaccaggccaagagggagattcgcttcctagagctacagaaggttgcctcctcttccagtgggaacaactttc
tctcaggttctccagcttctcccatgggtgatatcctgcagaccccacagttccagatgagacggctgaagaagcag
cttgctgatgagagaagtaatagggatgagctggagctggagctagctgagaaccgcaagctcctcaccgagaagga
tgcacagatagccatgatgcagcagcgcattgaccgcctagccctgctgaatgagaagcaggcggccagcccactgg
agcccaaggagcttgaggagctgcgtgacaagaatgagagccttaccatgcggctgcatgaaaccctgaagcagtgc
caggacctgaagacagagaagagccagatggatcgcaaaatcaaccagctttcggaggagaatggagacctttcctt
taagctgcgggagtttgccagtcatctgcagcagctacaggatgccctcaatgagctgacggaggagcacagcaagg
ccactcaggagtggctagagaagcaggcccagctggagaaggagctcagcgcagccctgcaggacaagaaatgcctt
gaagagaagaacgaaatccttcagggaaaactttcacagctggaagaacacttgtcccagctgcaggataacccacc
ccaggagaagggcgaggtgctgggtgatgtcttgcagctggaaaccttgaagcaagaggcagccactcttgctgcaa
acaacacacagctccaagccagggtagagatgctggagactgagcgaggccagcaggaagccaagctgcttgctgag
cggggccacttcgaagaagaaaagcagcagctgtctagcctgatcactgacctgcagagctccatctccaacctcag
ccaggccaaggaagagctggagcaggcctcccaggctcatggggcccggttgactgcccaggtggcctctctgacct
ctgagctcaccacactcaatgccaccatccagcaacaggatcaagaactggctggcctgaagcagcaggccaaagag
aagcaggcccagctagcacagaccctccaacagcaagaacaggcctcccagggcctccgccaccaggtggagcagct
aagcagtagcctgaagcagaaggagcagcagttgaaggaggtagcggagaagcaggaggcaactaggcaggaccatg
cccagcaactggccactgctgcagaggagcgagaggcctccttaagggagcgggatgcggctctcaagcagctggag
gcactggagaaggagaaggctgccaagctggagattctgcagcagcaacttcaggtggctaatgaagcccgggacag
tgcccagacctcagtgacacaggcccagcgggagaaggcagagctgagccggaaggtggaggaactccaggcctgtg
ttgagacagcccgccaggaacagcatgaggcccaggcccaggttgcagagctagagttgcagctgcggtctgagcag
caaaaagcaactgagaaagaaagggtggcccaggagaaggaccagctccaggagcagctccaggccctcaaagagtc
cttgaaggtcaccaagggcagccttgaagaggagaagcgcagggctgcagatgccctggaagagcagcagcgttgta
tctctgagctgaaggcagagacccgaagcctggtggagcagcataagcgggaacgaaaggagctggaagaagagagg
gctgggcgcaaggggctggaggctcgattactgcagcttggggaggcccatcaggctgagactgaagtcctgcggcg
ggagctggcagaggccatggctgcccagcacacagctgagagtgagtgtgagcagctcgtcaaagaagtagctgcct
ggcgtgacgggtatgaggatagccagcaagaggaggcacagtatggcgccatgttccaggaacagctgatgactttg
aaggaggaatgtgagaaggcccgccaggagctgcaggaggcaaaggagaaggtggcaggcatagaatcccacagcga
gctccagataagccggcagcagaacaaactagctgagctccatgccaacctggccagagcactccagcaggtccaag
agaaggaagtcagggcccagaagcttgcagatgacctctccactctgcaggaaaagatggctgccaccagcaaagag
gtggcccgcttggagaccttggtgcgcaaggcaggtgagcagcaggaaacagcctcccgggagttagtcaaggagcc
tgcgagggcaggagacagacagcccgagtggctggaagagcaacagggacgccagttctgcagcacacaggcagcgc
tgcaggctatggagcgggaggcagagcagatgggcaatgagctggaacggctgcgggccgcgctgatggagagccag
gggcagcagcaggaggagcgtgggcagcaggaaagggaggtggcgcggctgacccaggagcggggccgtgcccaggc
tgaccttgccctggagaaggcggccagagcagagcttgagatgcggctgcagaacgccctcaacgagcagcgtgtgg
agttcgctaccctgcaagaggcactggctcatgccctgacggaaaaggaaggcaaggaccaggagttggccaagctt
cgtggtctggaggcagcccagataaaagagctggaggaacttcggcaaaccgtgaagcaactgaaggaacagctggc
taagaaagaaaaggagcacgcatctggctcaggagcccaatctgaggctgctggcaggacagagccaacaggcccca
agctggaagcactgcgggcagaggtgagcaagctggaacagcaatgccagaagcagcaggagcaggctgacagcctg
gaacgcagcctcgaggctgagcgggcctcccgggctgagcgggacagtgctctggagactctgcagggccagttaga
ggagaaggcccaggagctagggcacagtcagagtgccttagcctcggcccaacgggagttggctgccttccgcacca
aggtacaagaccacagcaaggctgaagatgagtggaaggcccaggtggcccggggccggcaagaggctgagaggaaa
aatagcctcatcagcagcttggaggaggaggtgtccatcctgaatcgccaggtcctggagaaggagggggagagcaa
ggagttgaagcggctggtgatggccgagtcagagaagagccagaagctggaggagagctgcgcctgctgcaggcaga
gacagccagcaacagtgccagagctgcagaacgcagctctgctctgcgggaggaggtgcagagcctccgggagggag
gctgagaaacagcgggtggcttcagagaacctgcggcaggagctgacctcacaggctgagcgtgcggaggagctggg
ccaagaattgaaggcgtggcaggagaagttcttccagaaagagcaggccctctccaccctgcagctcgagcacacca
gcacacaggccctggtgagtgagctgctgccagctaagcacctctgccagcagctgcaggccgagcaggccgctgcc
gagaaacgccaccgtgaggagctggagcagagcaagcaggccgctgggggactgcgggcagagctgctgcgggccca
gcgggagcttggggagctgattcctctgcggcagaaggtggcagagcaggagcgaacagctcagcagctgcgggcag
agaaggccagctatgcagagcagctgagcatgctgaagaaggcgcatggcctgctggcagaggagaaccgggggctg
ggtgagcgggccaaccttggccggcagtttctggaagtggagttggaccaggcccgggaaaagtatgtccaagagtt
ggcagccgtacgtgctgatgctgagacccgtctggctgaggtgcagcgagaagcacagagcactgcccgggagctgg
aggtgatgactgccaagtatgagggtgccaaggtcaaggtcctggaggagaggcagcggttccaggaagagaggcag
aaactcactgcccaggtggaagaactgagtaagaaactggctgactctgaccaagccagcaaggtgcagcagcagaa
gctgaaggctgtccaggctcagggaggcgagagccagcaggaggcccagcgcttccaggcccagctgaatgaactgc
aagcccagttgagccagaaggagcaggcagctgagcactataagctgcagatggagaaagccaaaacacattatgat
gccaagaagcagcagaaccaagagctgcaggagcagctgcggagcctggagcagctgcagaaggaaaacaaagagct
gcgagctgaagctgaacggctgggccatgagctacagcaggctgggctgaagaccaaggaggctgaacagacctgcc
gccaccttactgcccaggtgcgcagcctggaggcacaggttgcccatgcagaccagcagcttcgagacctgggcaaa
ttccaggtggcaactgatgctttaaagagccgtgagccccaggctaagccccagctggacttgagtattgacagcct
ggatctgagctgcgaggaggggaccccactcagtatcaccagcaagctgcctcgtacccagccagacggcaccagcg
tccctggagaaccagcctcacctatctcccagcgcctgccccccaaggtagaatccctggagagtctctacttcact
cccatccctgctcggagtcaggcccccctggagagcagcctggactccctgggagacgtcttcctggactcgggtcg
taagacccgctccgctcgtcggcgcaccacgcagatcatcaacatcaccatgaccaagaagctagatgtggaagagc
cagacagcgccaactcatcgttctacagcacgcggtctgctcctgcttcccaggctagcctgcgagccacctcctct
actcagtctctagctcgcctgggttctcccgattatggcaactcagccctgctcagcttgcctggctaccgccccac
cactcgcagttctgctcgtcgttcccaggccggggtgtccagtggggcccctccaggaaggaacagcttctacatgg
gcacttgccaggatgagcctgagcagctggatgactggaaccgcattgcagagctgcagcagcgcaatcgagtgtgc
cccccacatctgaagacctgctatcccctggagtccaggccttccctgagcctgggcaccatcacagatgaggagat
gaaaactggagacccccaagagaccctgcgccgagccagcatgcagccaatccagatagccgagggcactggcatca
ccacccggcagcagcgcaaacgggtctccctagagccccaccagggccctggaactcctgagtctaagaaggccacc
agctgtttcccacgccccatgactccccgagaccgacatgaagggcgcaaacagagcactactgaggcccagaagaa
agcagctccagcttctactaaacaggctgaccggcgccagtcgatggccttcagcatcctcaacacacccaagaagc
tagggaacagccttctgcggcggggagcctcaaagaaggccctgtccaaggcttcccccaacactcgcagtggaacc
cgccgttctccgcgcattgccaccaccacagccagtgccgccactgctgccgccattggtgccacccctcgagccaa
gggcaaggcaaagcactaaagggccagtaccagtgagtggccccacctgtgtccccgatgctgccgtcacctggtcc
tccgcctactgtccctctcagtgccttctctcagctcccaggccaacagtagccaaacccctagagacagtgatgcc
tgcccgcaccctggcctggcccctggtccttcactggcgccttctcggagctggcccagggggcctggagcatggac
agtgtgggcgctctccctaccttgcctccttttttcttaaagcaaagtcacttctccatcacaaccagatttgaggc
tggttttgatggctgggtccttgggcctggccagtcttcctcttagcctctggatctagaagggaccataagaggag
taggccctggttcctgctgtcctggtggctgggccagcaggggccctcactcttgaagtccaggactgggtctgacc
tggtgggagcacctgccagaggatgctctttcccaggacggatgggccctgtgtctcaggagtggggttgggggaca
gccttcagcagcagctcacaccctaccttccccagacttgcactggggtgggatttggagtgatgggaaggttttta
agggccggggatggatcttttctaaatgttattacttgtaaataaagtctattttt

Following is a GALE cDNA sequence (SEQ ID NO: 15).

NM_000403.2 [GI: 9945333] Homo sapiens galactose-4-epimerase, UDP-(GALE),
mRNA.
gactctccagtcctcagtcaccttggacaaagaagtgtggatcctcagattccatcttttccaactccaaggtgcca
tggcagagaaggtgctggtaacaggtggggctggctacattggcagccacacggtgctggagctgctggaggctggc
tacttgcctgtggtcatcgataacttccataatgccttccgtggagggggctccctgcctgagagcctgcggcgggt
ccaggagctgacaggccgctctgtggagtttgaggagatggacattttggaccagggagccctacagcgtctcttca
aaaagtacagctttatggcggtcatccactttgcggggctcaaggccgtgggcgagtcggtgcagaagcctctggat
tattacagagttaacctgaccgggaccatccagcttctggagatcatgaaggcccacggggtgaagaacctggtgtt
cagcagctcagccactgtgtacgggaacccccagtacctgccccttgatgaggcccaccccacgggtggttgtacca
acccttacggcaagtccaagttcttcatcgaggaaatgatccgggacctgtgccaggcagacaagacttggaacgta
gtgctgctgcgctatttcaaccccacaggtgcccatgcctctggctgcattggtgaggatccccagggcatacccaa
caacctcatgccttatgtctcccaggtggcgatcgggcgacgggaggccctgaatgtctttggcaatgactatgaca
cagaggatggcacaggtgtccgggattacatccatgtcgtggatctggccaagggccacattgcagccttaaggaag
ctgaaagaacagtgtggctgccggatctacaacctgggcacgggcacaggctattcagtgctgcagatggtccaggc
tatggagaaggcctctgggaagaagatcccgtacaaggtggtggcacggcgggaaggtgatgtggcagcctgttacg
ccaaccccagcctggcccaagaggagctggggtggacagcagccttagggctggacaggatgtgtgaggatctctgg
cgctggcagaagcagaatccttcaggctttggcacgcaagcctgaggaccctcccctaccaaggaccaggaaaagca
gcagctgcctgctctccagcctctggaggaactcagggccctggagctgctggggccaagccaagggcctcccctac
ctcaaaccccagctgggcccgcttagcccaccaggcatgaggccaaggctccactgaccaggaggccgaggtctcta
actcttatcttccacagggtccaagagttcatcaggacccccaagagtgagtgagggggcaaggctctggcacaaaa
cctcctcctcccaggcactcatttatattgctctgaaagagctttccaaagtatttaaaaataaaaacaagttttct
tacactgg

Following is a DPF3 cDNA sequence (SEQ ID NO: 16).

NM_012074 [gi: 13442997] Homo sapiens D4, zinc and double PHD fingers,
family 3 (DPF3), mRNA.
acattgtagcaaaatggcgactgtcattcacaaccccctgaaagcgctcggggaccagttctacaaggaagccattg
agcactgccggagttacaactcacggctgagtgcagagcgcagcgtgcgtcttcccttcctggactcacagactggg
gtggcccagaacaactgctacatctggatggagaagaggcaccgaggcccaggccttgccccgggccagctgtatac
ataccctgcccgctgctggcgcaagaagagacgattgcacccacctgaagatccaaaactgcggctgctggagataa
aacctgaagtggagcttcccctgaagaaggatgggttcacctcagagagcaccacgctggaagccttgctccgtggc
gagggggttgagaagaaggtggatgccagggaggaggaaagcatccaggaaatacagagggttttggaaaatgatga
aaatgtagaagaagggaatgaagaagaggatttggaagaggatattcccaagcgaaaggacaggactagaggacggg
ctcgctgccctctcccttccctgcactgtttttcctcccttccctctgccgtgatagatgctaaggagtggggtgga
ggtgggaagtgggaagcaacagtggcgtataggaaaaagaaaatataccccgtgcacattttcaacatgtagttgaa
gaagcctaaattaggtactagaaaaaaaaaaaggacagaaacactgcctgatatgtgagcaagagcatgaaaatata
gaatgattatctcagagcagaggaggtggggagcaggtggctggagagaggcggggtaggtaggcagggcgtccctg
gctcaccaatgcgtctggtccacatggcatttgggaaagcagatgggtcatgcttttgcaatgaatggtcagatgct
tagggggcatttgtgctcctctggccaggaaagggaacagagtccatccaagctgccccccactcatctgccatggt
tgtccaccctgggggtttttcctctgaaggaaaatgaacccatcttttgcctgccatgaatcattgcagggcaggct
gttactggctaaattaaaacccttggcagggcatcgactgtgtgcgaggcaatgctctaggtcctgtaggggctgga
aaataaaccgcatgctggcctagccttcaagttgcttttcagccatgaaactaagctctgccaagcaatcgtgattg
taggtcaagtatcgcgccatcacggaaggtgtaagtatatgtaggtttctctgttgaggatgcgtgttcctcagtag
aagacaagcaggtggaggcatccagtgatttctaccctgtggaggctgaggggtcgggggaagaaaacagatgacct
cagcctagttgctttaatctgcttttccaagcactggatgcccttggatgacagcatcctcagcattaaaactggtg
aactgatgaagtcacctggcctggagtgtgttgggcagccagtgtccccagagctgcttgtgggtttctggggtgga
aggcagggaggtgcaactggcagggcctgatcagaggcagaaaatgacccccacagtggtcttttccctgctagaga
aagcagagagcgggactgggggggtgggggcttcaagtacagattgggcacactccaccagaccccagcaaggtcag
ctgccccacgcctgtatctggcactgctggtgtgtgcagggatgaaacccagcatcagagaggttttcagcaaacct
tccatgctcacctttggccaggtgcttgtcagatcctagcttcgtgctggcttagactctcagttgtttgtgttgac
agcattaggaataaaccgtttgtttcatctttccttttctccacacgtgtcacagccagatttcagctttgagttat
tccctgaagaagccacaccaaccttgctttcaagcaaaatgcctgggcttgggggaaggtgtgtatctgtccatgtg
tggatgtcggctcagagctatagcttctctgtggggggtggcccaagggaaggctcctctggggccctggatggcac
atgactcccagtgaggagaattcaggtgatctctgtggaggtagtcagggacacaaggcttggctgtgagtctggtt
ttaaagtgcgtgacagcctgaaaagcatgcaggggtttggtccactcacctacttgaaagcctgtgggcaacgttct
tttgagccaagacttctctgaatggccctgctggtggaaggggtgaggcaaaggcctctgacttggaccctttccac
accagactggcagcacttcccccaggcagccagtggtgggccctgagccctcaggtccccagctccttgagggatga
acctgggagcccaagagccagtggctgagctctgagaaggctccatctcccacctgcccttgagcgcgctctcaggc
tgagaacacggtctcatcaggcgccttcctggcctgatgctgtgctgtctacgtcacacggtcgattcacaaaagcc
agaactagacctcaaccaggtcatctccccgttgccaagtgggttcagggtgagggcaatttgtaagctcaatttct
ctgacagccaagacatggagcatctctgctaagaagccaaaagaaattggttttcttcttctcattgctgaagcccc
tctgtgtctcttctcagggacaggctggtccagtggctttggtgagggcgcctccatttgtgaacgctgggattcct
ctgggtgggctttggcaggtggctcctatggtaggaaggatgaccaggtccctgggaatgagctgcctacctgctgc
tccacgaggaagctcaggcctgcacaggctccaccaggccttggatgccctctagttgagtcagagaccctggaaac
acactgagatctccaattgctgcctccattgatgtctctagacctgcagatacgaagcaaacctgggattgcttctt
ccaggtatgggcaccagagagggaggccactgcaacattttatccccatcattccaaaatgcttgcttgtctctttt
acctcacactcacaactccttctgagactctcagtcataaaggaatgaccaagagagtgggtctccagtgagagaaa
tgcctatgaaagagggtttccctttttgctcttttgaacaccctccccactgatccttgggacccaacgccgcattg
cctcttgcagatgaggttttgccttgggctgcttgggtacttcagaccaggactgagtctgacacagctttcatgag
gttacagaaaagggctacagatttgggaagctgtgtgtaatggtcttgagacaatatctccatttggcccaccctgg
cttctctaaaaagcaacgacagcaacagacaaacaaaaagctcccacctcccaccccgttagctgtcctcctccttc
actgtgatgtggttgcggtctctgtaggtgtgtgtgccacccttgtcctctgtcctctggggatgtgcccttcccac
gtgtgtcaggttcccactctttcgtggttcctaacgtgaagtgctgtgatgtttctgccctgcctaaggaacgtatc
aagctctctcagtgtttcagtgttggagattgaggctgtgccacatcttctgccatcctaaggggacatgatggttc
tgtgattcccagagagctggcagattgtgacaatctccaggagaacctacagattggaagcagcccacacctgatgt
ggactcctgtcccgggactcactcttcattcagaagactggtggcccacgtgccaggaccaccccacctctcttgct
gccttttctcctgtcctgatggggttctgggagggagacctgtcgctgatgagatgaagaatgtggggatcgagcag
ccttcttctttgggacccctcgatatcccatggaatgctcgcacgttctcaaagactgagtcacaagcccctacccc
ttccttgctgtggttagtatcttgttctgtgattggttagcaatgttgactacccacgtagtgaatcttttgtctgc
aatttagagaatgtgtaaacaaataaaaggctttaaaactc

Following is a LOC145197 cDNA sequence (SEQ ID NO: 17).

XM_096734.1 GI: 18582778; Homo sapiens
hypothetical LOC145197 (LOC145197), mRNA.
agtgcaatgagcttcatgctgaatactgatttgaagtgtggttcaccaca
cagacttaaattgtccttcccacaactttatctgagaagcatgacaacgg
tgacaatcacaggactagttttattggtcaggatgtggtgttactatatt
aaagcgcttcaagttttcatagcgctcgtcaaacagagacctcggaacgc
tctgcagacattaattaatgttcgctccggccctggaagccatttcacca
atggaaattccgatgagaaaggaagaaacgaggggttcaaggccgcgccg
ttgtgagtccgctgcggaactaggaactgaaaccccgtggttcccaatga
gccagcgaaagtctgtcgaagtattgagcacccctattgaagactgtggg
agttttggcggcttaatgggattgtgttcggtccttcaggctctccttta
aaagattccatggcaccaacgagactccaataaaagggcctctctaa

Following are ICAM, MAPK10, KIAA0861, NUMA, GALE, DPF3 and LOC145197 amino acid sequences (SEQ ID NO: 18, 19, 20, 21, 22, 23, 24, 25 and 26, respectively). Following is an ICAM1 cDNA sequence (SEQ ID NO: 18).

NP_000192 [gi: 4557878] intercellular adhesion
molecule 1 precursor; CD54 [Homo sapiens]
MAPSSPRPALPALLVLLGALFPGPGNAQTSVSPSKVILPRGGSVLVTCST
SCDQPKLLGIETPLPKKELLLPGNNRKVYELSNVQEDSQPMCYSNCPDGQ
STAKTFLTVYWTPERVELAPLPSWQPVGKNLTLRCQVEGGAPRANLTVVL
LRGEKELKREPAVGEPAEVTTTVLVRRDHHGANFSCRTELDLRPQGLELF
ENTSAPYQLQTFVLPATPPQLVSPRVLEVDTQGTVVCSLDGLFPVSEAQV
HLALGDQRLNPTVTYGNDSFSAKASVSVTAEDEGTQRLTCAVILGNQSQE
TLQTVTIYSFPAPNVILTKPEVSEGTEVTVKCEAHPRAKVTLNGVPAQPL
GPRAQLLLKATPEDNGRSFSCSATLEVAGQLIHKNQTRELRVLYGPRLDE
RDCPGNWTWPENSQQTPMCQAWGNPLPELKCLKDGTFPLPIGESVTVTRD
LEGTYLCRARSTQGEVTREVTVNVLSPRYEIVIITVVAAAVIMGTAGLST
YLYNRQRKIKKYRLQQAQKGTPMKPNTQATPP

Following is an ICAM4 amino acid sequence (SEQ ID NO: 19).

NP_001535 [gi: 4504561] intercellular adhesion
molecule 4 isoform 1 precursor; Landsteiner-
Wiener blood group protein [Homo sapiens].
MGSLFPLSLLFFLAAAYPGVGSALGRRTKRAQSPKGSPLAPSGTSVPFWV
RMSPEFVAVQPGKSVQLNCSNSCPQPQNSSLRTPLRQGKTLRGPGWVSYQ
LLDVRAWSSLAHCLVTCAGKTRWATSRITAYKPPHSVILEPPVLKGRKYT
LRCHVTQVFPVGYLVVTLRHGSRVIYSESLERFTGLDLANVTLTYEFAAG
PRDFWQPVICHARLNLDGLVVRNSSAPITLMLAWSPAPTALASGSIAALV
GILLTVGAAYLCKCLAMKSQA

Following is an ICAM5 amino acid sequence (SEQ ID NO: 20).

NP_003250 [gi: 12545404] intercellular adhesion
molecule 5 precursor; telencephalin [Homo sapiens].
MPGPSPGLRRALLGLWAALGLGLFGLSAVSQEPFWADLQPRVAFVERGGS
LWLNCSTNCPRPERGGLETSLRRNGTQRGLRWLARQLVDIREPETQPVCF
FRCARRTLQARGLIRTFQRPDRVELMPLPPWQPVGENFTLSCRVPGAGPR
ASLTLTLLRGAQELIRRSFAGEPPRARGAVLTATVLARREDHGANFSCRA
ELDLRPHGLGLFENSSAPRELRTFSLSPDAPRLAAPRLLEVGSERPVSCT
LDGLFPASEARVYLALGDQNLSPDVTLEGDAFVATATATASAEQEGARQL
ICNVTLGGENRETRENVTIYSFPAPLLTLSEPSVSEGQMVTVTCAAGTQA
LVTLEGVPAAVPGQPAQLQLNATENDDRRSFFCDATLDVDGETLIKNRSA
ELRVLYAPRLDDSDCPRSWTWPEGPEQTLRCEARGNPEPSVHCARSDGGA
VLALGLLGPVTRALSGTYRCKAANDQGEAVKDVTLTVEYAPALDSVGCPE
RITWLEGTEASLSCVAHGVPPPDVICVRSGELGAVIEGLLRVAREHAGTY
RCEATNPRGSAAKNVAVTVEYGPRFEEPSCPSNWTWVEGSGRLFSCEVDG
KPQPSVKCVGSGGATEGVLLPLAPPDPSPRAPRIPRVLAPGIYVCNATNR
HGSVAKTVVVSAESPPEMDESTCPSHQTWLEGAEASALACAARGRPSPGV
RCSREGIPWPEQQRVSREDAGTYHCVATNAHGTDSRTVTVGVEYRPVVAE
LAASPPGGVRPGGNFTLTCRAEAWPPAQISWRAPPGALNIGLSSNNSTLS
VAGAMGSHGGEYECARTNAHGRHARRITVRVAGPWLWVAVGGAAGGAALL
AAGAGLAFYVQSTACKKGEYNVQEAESSGEAVCLNGAGGGAGGAAGAEGG
PEAAGGAAESPAEGEVFAIQLTSA

Following is a MAPK10 amino acid sequence (SEQ ID NO: 21).

NP_002744 [gi: 4506081] mitogen-activated
protein kinase 10 isoform 1; JNK3 alpha protein
kinase; stress activated protein kinase beta; MAP
kinase; c-Jun kinase 3; c-Jun N-terminal kinase 3;
stress activated protein kinase JNK3
[Homo sapiens].
MSLHFLYYCSEPTLDVKIAFCQGFDKQVDVSYIAKHYNMSKSKVDNQFYS
VEVGDSTFTVLKRYQNLKPIGSGAQGIVCAAYDAVLDRNVAIKKLSRPFQ
NQTHAKRAYRELVLMKCVNHKNIISLLNVFTPQKTLEEFQDVYLVMELMD
ANLCQVIQMELDHERMSYLLYQMLCGIKHLHSAGIIHRDLKPSNIVVKSD
CTLKILDFGLARTAGTSFMMTPYVVTRYYRAPEVILGMGYKENVDIWSVG
CIMGEMVRHKILFPGRDYIDQWNKVIEQLGTPCPEFMKKLQPTVRNYVEN
RPKYAGLTFPKLFPDSLFPADSEHNKLKASQARDLLSKMLVIDPAKRISV
DDALQHPYINVWYDPAEVEAPPPQIYDKQLDEREHTIEEWKELIYKEVMN
SEEKTKNGVVKGQPSPSAQVQQ

Additional isoforms are isoform 2 (NP_{—}620448); isoform 3 (NP_{—}620446); isoform 4 (NP_{—}620447)

Following is a first KIAA0861 amino acid sequence (SEQ ID NO: 22).

### KIAA0861 Polypeptide Sequence Corresponding to SEQ ID NO: 3.

MTYLTSIPSVEAASIGFIVVIDRRRDKWSSVKASLTRIAVAFPGNLQLIFILRPSRFIQRTFTDIGIKYYRNEFKTK
VPIIMVNSVSDLHGYIDKSQLTRELGGTLEYRHGQWVNHRTAIENFALTLKTTAQMLQTFGSCLATAELPRSMLSTE
DLLMSHTRQRDKLQDELKLLGKQGTTLLSCIQEPATKCPNSKLNLNQLENVTTMERLLVQLDETEKAFSHFWSEHHL
KLNQCLQLQHFEHDFCKAKLALDNLLEEQAEFTGIGDSVMHVEQILKEHKKLEEKSQESLEKAQLLALVGDQLIQSH
HYAADAIRPRCVELRHLCDDFINGNKKKWDILGKSLEFHRQLDKVSQWCEAGIYLLASQAVDKCQSREGVDIALNDI
ATFLGTVKEYPLLSPKEFYNEFELLLTLDAKAKAQKVLQRLDDVQEIFHKRQVSLMKLAAKQTRPVQPVAPHPESSP
KWVSSKTSQPSTSVPLARPLRTSEEPYTETELNSRGKEDDETKFEVKSEEIFESHHERGNPELEQQARLGDLSPRRR
IIRDLLETEEIYIKEIKSIIDGYITPMDFIWLKHLIPDVLQNNEDFLFGNIRELYEFHNRTFLKELEKCAENPELLA
HCFLKRKEDLQIYFKYHKNLPRARAIWQECQDCAYFGVCQRQLDHNLPLFKYLKGPSQRLIKYQMLLKGLLDFESPE
DMEIDPGELGGSAKDGPKRTKDSAFSTELQQALAVIEDLIKSCELAVDLAAVTECPDDIGKLGKLLLHGPFSVWTIH
KDRYKMKDLIRFKPSQRQIYLFERGIVFCKIRMEPGDQGLSPHYSFKKAMKLMTLSIRQLGRGSHRKFEIASRNGLE
KYILQAASKEIRDCWFSEISKLLMEQQNNIKDQGNPQFEMSTSKGSGAGSGPWIKNMERATTSKEDPASSTGGIKGC
SSREFSSMDTFEDCEGAEDMEKESSALSLAGLFQSDDSHETCSSKSAFLERGESSQGEKEERDEEETATRSTEEERA
GASTGRLAPAGATAGFQARALRPRTSAQES (1031 aa)

Following is a second KIAA0861 amino acid sequence (SEQ ID NO: 27).

NP_055893: KIAA0861
MLSCLKEEMPPQELTRRLATVITHVDEIMQQEVRPLMAVEIIEQLHRQFAILSGGRGEDGAPIITFPEFSGFKHIPD
EDFLNVMTYLTSIPSVEAASIGFIVVIDRRRDKWSSVKASLTRIAVAFPGNLQLIFILRPSRFIQRTFTDIGIKYYR
NEFKTKVPIIMVNSVSDLHGYIDKSQLTRELGGTLEYRHGQWVNHRTAIENFALTLKTTAQMLQTFGSCLATAELPR
SMLSTEDLLMSHTRQRDKLQDELKLLGKQGTTLLSCIQEPATKCPNSKLNLNQLENVTTMERLLVQLDETEKAFSHF
WSEHHLKLNQCLQLQHFEHDFCKAKLALDNLLEEQAEFTGIGDSVMHVEQILKEHKKLEEKSQEPLEKAQLLALVGD
QLIQSHHYAADAIRPRCVELRHLCDDFINGNKKKWDILGKSLEFHRQLDKVSQWCEAGIYLLASQAVDKCQSREGVD
IALNDIATFLGTVKEYPLLSPKEFYNEFELLLTLDAKAKAQKVLQRLDDVQEIFHKRQVSLMKLAAKQTRPVQPVAP
HPESSPKWVSSKTSQPSTSVPLARPLRTSEEPYTETELNSRGKEDDETKFEVKSEEIFESHHERGNPELEQQARLGD
LSPRRRIIRDLLETEEIYIKEIKSIIDGYITPMDFIWLKHLIPDVLQNNKDFLFGNIRELYEFHNRTFLKELEKCAE
NPELLAHCFLKRKEDLQIYFKYHKNLPRARAIWQECQDCAYFGVCQRQLDHNLPLFKYLKGPSQRLIKYQMLLKGLL
DFESPEDMEIDPGELGGSAKDGPKRTKDSAFSTELQQALAVIEDLIKSCELAVDLAAVTECPDDIGKLGKLLLHGPF
SVWTIHKDRYKMKDLIRFKPSQRQIYLFERGIVFCKIRMEPGDQGLSPHYSFKKEMKLMTLSIRQLGRGSHRKFEIA
SRNGLEKYILQAASKEIRDCWFSEISKLLMEQQNNIKDQGNPQFEMSTSKGSGAGSGPWIKNMERATTSKEDPASST
GGIKGCSSREFSSMDTFEDCEGAEDMEKESSALSLAGLFQSDDSHETCSSKSAFLERGESSQGEKEERDEEETATRS
TEEERAGASTGRLAPAGATAGFQARALRPRTSAQES (1114 aa)

Following is a NUMA1 amino acid sequence (SEQ ID NO: 24).

NP_006176 [gi: 5453820] nuclear mitotic apparatus protein 1 [Homo sapiens].
MTLHATRGAALLSWVNSLHVADPVEAVLQLQDCSIFIKIIDRIHGTEEGQQILKQPVSERLDFVCSFLQKNRKHPSS
PECLVSAQKVLEGSELELAKMTMLLLYHSTMSSKSPRDWEQFEYKIQAELAVILKFVLDHEDGLNLNEDLENFLQKA
PVPSTCSSTFPEELSPPSHQAKREIRFLELQKVASSSSGNNFLSGSPASPMGDILQTPQFQMRRLKKQLADERSNRD
ELELELAENRKLLTEKDAQIAMMQQRIDRLALLNEKQAASPLEPKELEELRDKNESLTMRLHETLKQCQDLKTEKSQ
MDRKINQLSEENGDLSFKLREFASHLQQLQDALNELTEEHSKATQEWLEKQAQLEKELSAALQDKKCLEEKNEILQG
KLSQLEEHLSQLQDNPPQEKGEVLGDVLQLETLKQEAATLAANNTQLQARVEMLETERGQQEAKLLAERGHFEEEKQ
QLSSLITDLQSSISNLSQAKEELEQASQAHGARLTAQVASLTSELTTLNATIQQQDQELAGLKQQAKEKQAQLAQTL
QQQEQASQGLRHQVEQLSSSLKQKEQQLKEVAEKQEATRQDHAQQLATAAEEREASLRERDAALKQLEALEKEKAAK
LEILQQQLQVANEARDSAQTSVTQAQREKAELSRKVEELQACVETARQEQHEAQAQVAELELQLRSEQQKATEKERV
AQEKDQLQEQLQALKESLKVTKGSLEEEKRRAADALEEQQRCISELKAETRSLVEQHKRERKELEEERAGRKGLEAR
LLQLGEAHQAETEVLRRELAEAMAAQHTAESECEQLVKEVAAWRDGYEDSQQEEAQYGAMFQEQLMTLKEECEKARQ
ELQEAKEKVAGIESHSELQISRQQNKLAELHANLARALQQVQEKEVRAQKLADDLSTLQEKMAATSKEVARLETLVR
KAGEQQETASRELVKEPARAGDRQPEWLEEQQGRQFCSTQAALQAMEREAEQMGNELERLRAALMESQGQQQEERGQ
QEREVARLTQERGRAQADLALEKAARAELEMRLQNALNEQRVEFATLQEALAHALTEKEGKDQELAKLRGLEAAQIK
ELEELRQTVKQLKEQLAKKEKEHASGSGAQSEAAGRTEPTGPKLEALRAEVSKLEQQCQKQQEQADSLERSLEAERA
SRAERDSALETLQGQLEEKAQELGHSQSALASAQRELAAFRTKVQDHSKAEDEWKAQVARGRQEAERKNSLISSLEE
EVSILNRQVLEKEGESKELKRLVMAESEKSQKLEESCACCRQRQPATVPELQNAALLCGRRCRASGREAEKQRVASE
NLRQELTSQAERAEELGQELKAWQEKFFQKEQALSTLQLEHTSTQALVSELLPAKHLCQQLQAEQAAAEKRHREELE
QSKQAAGGLRAELLRAQRELGELIPLRQKVAEQERTAQQLRAEKASYAEQLSMLKKAHGLLAEENRGLGERANLGRQ
FLEVELDQAREKYVQELAAVRADAETRLAEVQREAQSTARELEVMTAKYEGAKVKVLEERQRFQEERQKLTAQVEEL
SKKLADSDQASKVQQQKLKAVQAQGGESQQEAQRFQAQLNELQAQLSQKEQAAEHYKLQMEKAKTHYDAKKQQNQEL
QEQLRSLEQLQKENKELRAEAERLGHELQQAGLKTKEAEQTCRHLTAQVRSLEAQVAHADQQLRDLGKFQVATDALK
SREPQAKPQLDLSIDSLDLSCEEGTPLSITSKLPRTQPDGTSVPGEPASPISQRLPPKVESLESLYFTPIPARSQAP
LESSLDSLGDVFLDSGRKTRSARRRTTQIINITMTKKLDVEEPDSANSSFYSTRSAPASQASLRATSSTQSLARLGS
PDYGNSALLSLPGYRPTTRSSARRSQAGVSSGAPPGRNSFYMGTCQDEPEQLDDWNRIAELQQRNRVCPPHLKTCYP
LESRPSLSLGTITDEEMKTGDPQETLRRASMQPIQIAEGTGITTRQQRKRVSLEPHQGPGTPESKKATSCFPRPMTP
RDRHEGRKQSTTEAQKKAAPASTKQADRRQSMAFSILNTPKKLGNSLLRRGASKKALSKASPNTRSGTRRSPRIATT
TASAATAAAIGATPRAKGKAKH

Following is a GALE amino acid sequence (SEQ ID NO: 25).

MAEKVLVTGGAGYIGSHTVLELLEAGYLPVVIDNFHNAFRGGGSLPESLR
RVQELTGRSVEFEEMDILDQGALQRLFKKYSFMAVIHFAGLKAVGESVQK
PLDYYRVNLTGTIQLLEIMKAHGVKNLVFSSSATVYGNPQYLPLDEAHPT
GGCTNPYGKSKFFIEEMIRDLCQADKTWNVVLLRYFNPTGAHASGCIGED
PQGIPNNLMPYVSQVAIGRREALNVFGNDYDTEDGTGVRDYIHVVDLAKG
HIAALRKLKEQCGCRIYNLGTGTGYSVLQMVQAMEKASGKKIPYKVVARR
EGDVAACYANPSLAQEELGWTAALGLDRMCEDLWRWQKQNPSGFGTQA

Following is a DPF3 amino acid sequence (SEQ ID NO: 26).

NP_036206 [gi: 13442998] cer-d4 (mouse)
homolog; 2810403B03Rik [Homo sapiens].
MATVIHNPLKALGDQFYKEAIEHCRSYNSRLSAERSVRLPFLDSQTGVAQ
NNCYIWMEKRHRGPGLAPGQLYTYPARCWRKKRRLHPPEDPKLRLLEIKP
EVELPLKKDGFTSESTTLEALLRGEGVEKKVDAREEESIQEIQRVLENDE
NVEEGNEEEDLEEDIPKRKDRTRGRARCPLPSLHCFSSLPSAVIDAKEWG
GGGKWEATVAYRKKKIYPVHIFNM

Following is a LOC145197 amino acid sequence (SEQ ID NO: 27).

XP_096734.1 GI: 18582779: hypothetical protein
XP_096734 [Homo sapiens].
MSFMLNTDLKCGSPHRLKLSFPQLYLRSMTTVTITGLVLLVRMWCYYIKA
LQVFIALVKQRPRNALQTLINVRSGPGSHFTNGNSDEKGRNEGFKAAPL

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. Also, citation of the above publications or documents is not intended as an admission that any of the foregoing is pertinent prior art, nor does it constitute any admission as to the contents or date of these publications or documents. U.S. patents and other publications and documents referenced are incorporated herein by reference.

What is claimed is:

1. A method for determining whether a human subject is at an increased risk or decreased risk of breast cancer, which comprises:
(a) detecting in a nucleic acid of the human subject the presence of a polymorphic variant selected from the group consisting of a thymine corresponding to position 40889 of SEQ ID NO: 6, a guanine corresponding to position 173467 of SEQ ID NO: 6, a guanine corresponding to position 197612 of SEQ ID NO: 6, a cytosine corresponding to position 197854 of SEQ ID NO: 6, and a complement of the foregoing; or
(b) detecting in a nucleic acid of the human subject the presence of a polymorphic variant selected from the group consisting of a cytosine corresponding to position 40889 of SEQ ID NO: 6, a thymine corresponding to position 173467 of SEQ ID NO: 6, an adenine guanine corresponding to position 197612 of SEQ ID NO: 6, a thymine corresponding to position 197854 of SEQ ID NO: 6, and a complement of the foregoing;
whereby it is determined that the subject is at an increased risk of breast cancer based on the presence of one or more of the polymorphic variants of (a), and whereby it is deter mined that the subject is at a decreased risk of breast cancer based on the presence of one or more of the polymorphic variants of (b).

2. The method of claim 1, which further comprises obtaining the nucleic acid sample from the subject.

3. The method of claim 1, wherein detecting the presence of the one or more polymorphic variants comprises:
hybridizing an oligonucleotide to the nucleic acid from the subject, wherein the oligonucleotide is complementary to a nucleotide sequence in the nucleic acid and hybridizes to a region adjacent to the polymorphic variant;
extending the oligonucleotide in the presence of one or more nucleotides, yielding extension products: and
detecting the presence of a polymorphic variant in the extension products.

4. The method of claim 1, wherein the polymorphic variant detected is a thymine corresponding to position 40889 of SEQ ID NO: 6, or a complement thereof.

5. The method of claim 1, wherein the polymorphic variant detected is a guanine corresponding to position 173467 of SEQ ID NO: 6, or a complement thereof.

6. The method of claim 1, wherein the polymorphic variant detected is a guanine corresponding to position 197612 of SEQ ID NO: 6, or a complement thereof.

7. The method of claim 1, wherein the polymorphic variant detected is a cytosine corresponding to position 197854 of SEQ ID NO: 6, or a complement thereof.

8. The method of claim 1, wherein the polymorphic variant detected is a cytosine corresponding to position 40889 of SEQ ID NO: 6, or a complement thereof.

9. The method of claim 1, wherein the polymorphic variant detected is a thymine corresponding to position 173467 of SEQ ID NO: 6, or a complement thereof.

10. The method of claim 1, wherein the polymorphic variant detected is an adenine corresponding to position 197612 of SEQ ID NO: 6, or a complement thereof.

11. The method of claim 1, wherein the polymorphic variant detected is a thymine corresponding to position 197854 of SEQ ID NO: 6, or a complement thereof.

12. The method of claim 1, wherein the human subject is Caucasian.

13. A method for determining whether a breast cancer detection procedure is administered to a human subject, which comprises:
(a) detecting in a nucleic acid of the human subject the presence of a polymorphic variant selected from the group consisting of a thymine corresponding to position 40889 of SEQ ID NO: 6, a guanine corresponding to position 173467 of SEQ ID NO: 6, a guanine corresponding to position 197612 of SEQ ID NO: 6, a cytosine corresponding to position 197854 of SEQ ID NO: 6, and a complement of the foregoing; or
(b) detecting in a nucleic acid of the human subject the presence of a polymorphic variant selected from the group consisting of a cytosine corresponding to position 40889 of SEQ ID NO: 6, a thymine corresponding to position 173467 of SEQ ID NO: 6, an adenine guanine corresponding to position 197612 of SEQ ID NO: 6, a thymine corresponding to position 197854 of SEQ ID NO: 6, and a complement of the foregoing; and
administering a breast cancer detection procedure to a human subject determined to have an increased risk of breast cancer based on the presence of one or more of the polymorphic variants of (a), or not administering a breast cancer detection procedure to a human subject determined to have a decreased risk of breast cancer based on the presence of one or more of the polymorphic variants of (b).

14. The method of claim 13, which further comprises obtaining the nucleic acid sample from the subject.

15. The method of claim 13, wherein detecting the presence of the one or more polymorphic variants comprises:
hybridizing an oligonucleotide to the nucleic acid from the subject, wherein the oligonucleotide is complementary to a nucleotide sequence in the nucleic acid and hybridizes to a region adjacent to the polymorphic variant;
extending the oligonucleotide in the presence of one or more nucleotides, yielding extension products: and
detecting the presence of a polymorphic variant in the extension products.

16. The method of claim 13, wherein the breast cancer detection procedure is selected from the group consisting of a mammography, an early mammography program, a frequent mammography program, a biopsy procedure, a breast biopsy and biopsy from another tissue, a breast ultrasound and optionally ultrasound analysis of another tissue, breast magnetic resonance imaging (MRI) and optionally MRI analysis of another tissue, electrical impedance (T-scan) analysis of breast and optionally of another tissue, ductal lavage, nuclear medicine analysis, scintimammography, BRCA1 and/or BRCA2 sequence analysis results, thermal imaging of the breast and optionally of another tissue, and a combination of the foregoing.

17. The method of claim 13, wherein the polymorphic variant detected is a thymine corresponding to position 40889 of SEQ ID NO: 6, or a complement thereof.

18. The method of claim 13, wherein the polymorphic variant detected is a guanine corresponding to position 173467 of SEQ ID NO: 6, or a complement thereof.

19. The method of claim 13, wherein the polymorphic variant detected is a guanine corresponding to position 197612 of SEQ ID NO: 6, or a complement thereof.

20. The method of claim 13, wherein the polymorphic variant detected is a cytosine corresponding to position 197854 of SEQ ID NO: 6, or a complement thereof.

21. The method of claim 13, wherein the polymorphic variant detected is a cytosine corresponding to position 40889 of SEQ ID NO: 6, or a complement thereof.

22. The method of claim 13, wherein the polymorphic variant detected is a thymine corresponding to position 173467 of SEQ ID NO: 6, or a complement thereof.

23. The method of claim 13, wherein the polymorphic variant detected is an adenine corresponding to position 197612 of SEQ ID NO: 6, or a complement thereof.

24. The method of claim 13, wherein the polymorphic variant detected is a thymine corresponding to position 197854 of SEQ ID NO: 6, or a complement thereof.

25. The method of claim 13, wherein the human subject is Caucasian.
